(19) United States
(12) Patent Application Publication
(10) Pub. No.: US 2024/0190855 A1
(43) Pub. Date: Jun. 13, 2024

(54) CDK INHIBITORS AND METHODS OF USE THEREOF

(71) Applicant: Relay Therapeutics, Inc., Cambridge, MA (US)

(72) Inventors: Alexander M. Taylor, Cambridge, MA (US); Timothy F. Briggs, Cambridge, MA (US); Nicolas A. Pabon, Cambridge, MA (US); Jing He, Cambridge, MA (US); Andre Lescarbeau, Somerville, MA (US); Alessandro Boezio, Cambridge, MA (US); Catherine A. Evans, Cambridge, MA (US); Cary Griffin Fridrich, Cambridge, MA (US); Brian P. Kelley, Cambridge, MA (US); Elaine B. Krueger, Cambridge, MA (US); Ravi Kurukulasuriya, Cambridge, MA (US); Thomas H. McLean, Cambridge, MA (US)

(21) Appl. No.: 18/276,946

(22) PCT Filed: Feb. 11, 2022
(86) PCT No.: PCT/US22/16114

§ 371 Date: Aug. 11, 2023

Related U.S. Application Data
(60) Provisional application No. 63/292,337, filed on Dec. 21, 2021.
(60) Provisional application No. 63/250,473, filed on Sep. 30, 2021.
(60) Provisional application No. 63/192,627, filed on May 25, 2021.
(60) Provisional application No. 63/166,638, filed on Mar. 26, 2021.
(60) Provisional application No. 63/149,095, filed on Feb. 12, 2021.

(51) Int. Cl. C07D 413/12; C07D 231/40; C07D 401/12; C07D 403/12; C07D 405/14; C07D 413/14; C07D 417/04; C07D 417/12; C07D 417/14; C07D 471/04; C07D 487/04; C07D 491/052; C07D 495/04; C07D 498/04; C07D 513/04

(52) U.S. Cl. CPC C07D 413/12; C07D 231/40; C07D 401/12; C07D 403/12; C07D 405/14; C07D 413/14; C07D 417/04; C07D 417/12; C07D 417/14; C07D 471/04; C07D 487/04; C07D 491/052; C07D 495/04; C07D 498/04; C07D 513/04

(57) ABSTRACT

The present disclosure relates to novel compounds and pharmaceutical compositions thereof, and methods for inhibiting the activity of CDK enzymes with the compounds and compositions of the disclosure. The present disclosure further relates to, but is not limited to, methods for treating disorders associated with CDK signaling with the compounds and compositions of the disclosure.

### CROSS REFERENCE TO RELATED APPLICATIONS

This application claims the benefit of U.S. Provisional Application No. 63/149,095, filed Feb. 12, 2021, U.S. Provisional Application No. 63/166,638, filed Mar. 26, 2021, U.S. Provisional Application No. 63/192,627, filed May 25, 2021, U.S. Provisional Application No. 63/250,473, filed Sep. 30, 2021, and U.S. Provisional Application No. 63/292,337, filed Dec. 21, 2021, each of which is herein incorporated by reference in its entirety.

### BACKGROUND

Cyclin-dependent kinases (CDKs) are a family of serine/threonine kinases that are regulated by direct binding to cyclins. The initially-discovered CDKs (CDK1, CDK2, CDK4, CDK6) bind to cognate cyclins during specific cell cycle phases, activating their kinase activity and promoting cell cycle progression (Malumbres M. Genome Biology 2014). Related CDK family members (CDK7, CDK8, CDK9, CDK12, CDK13) are involved in other biological functions such as transcriptional control (Chou J., et al. Cancer Discovery 2020).

The cell cycle is initiated following mitogenic stimuli that signal for cyclin D expression, binding to CDK4/6, and kinase activation. The active CDK4/6-cyclin D complex mono-phosphorylates the retinoblastoma protein (RB), a tumor suppressor, to initiate cyclin E expression and formation of an active CDK2-cyclin E complex. Activated CDK2-cyclin E hyper-phosphorylates RB, triggering DNA replication, which is further promoted by CDK2-cyclin A. Finally, CDK1-cyclin B and CDK1-cyclin A coordinate segregation of duplicated DNA within the mother cell to complete cell division, and form two new daughter cells (Otto, T., and Sicinski, P. Nat Rev Cancer 2017)

As sustained cellular proliferation is one hallmark of cancer, alterations in pathways controlling cell cycle progression are frequently associated with cancer. Indeed, CCNE1 (gene encoding cyclin E1 protein) is among the most frequently amplified genes in variety of cancers including ovarian, endometrial, gastric, cervical, bladder, esophageal, lung, and breast cancers (Sanchez-Vega F., et al. Cell 2018; Cerami E., et al. Cancer Discovery 2012). The amplified CCNE1 gene, which leads to overexpression of cyclin E1 protein, is believed to be the oncogenic driver in those tumors due to increased CDK2-cyclin E activity. Notably, CCNE1 amplified or overexpressed tumor cells are dependent on CDK2 activity and thus provide the rationale for targeting CDK2 in this genetically defined patient population (McDonald E. R., et al Cell 2017; Au-Yeung G., et al. Clin Cancer Research 2016). Furthermore, CDK2 activation via Cyclin E1 amplification and overexpression is a common mechanism of resistance to several approved targeted therapies (such as CDK4/6 and HER2 modulators), and therefore supports combined targeting of CDK2 with other validated drivers in cancer (Turner N. C., et al. J Clin Oncology 2019; Herrera-Abreu M. T., et al. Cancer Research 2016; Scaltriti M., et al. PNAS 2011).

Multiple pan-CDK inhibitors with activity against CDK2 and other CDKs have shown evidence of clinical activity, however they have also shown significant hematopoietic and gastrointestinal toxicities likely due to their inhibition of CDK1 (Otto, T., and Sicinski, P., Nat. Review Cancer 2017; Kumar, K. S., et al. Blood 2015; Shapiro G. I., et al. Clin Cancer Research 2001). Whereas CDK2 activity may be dispensable for normal cell function, CDK1 activity is essential in all cells, especially in the highly proliferating cells of the gut and the hematopoietic system (Berthet C., et al. Current Biology 2003; Jayapal S. R., et al. Haematologica 2015; Santamaria D., et al. Nature 2007; Lu S., et al. Tox Sciences 2020).

### SUMMARY

In some embodiments, the present disclosure encompasses the recognition that there is a need for CDK-selective inhibitor compounds, e.g., CDK2-selective inhibitor compounds, and methods for treating cancers and other disorders with these compounds.

In some embodiments, the present disclosure provides a compound of formula I-A:

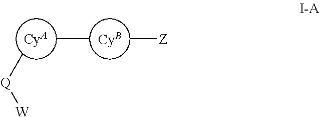

or a pharmaceutically acceptable salt thereof, wherein each of Cy^{A}, CY^{B}, Q, W, and Z is as defined in embodiments and classes and subclasses herein.

In some embodiments, the present disclosure provides a compound of formula I:

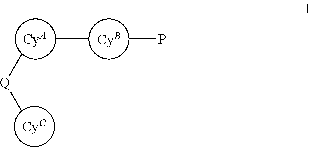

or a pharmaceutically acceptable salt thereof, wherein each of Cy^{A}, Cy^{B}, C^{C}, Q, and P is as defined in embodiments and classes and subclasses herein.

In some embodiments, the present disclosure provides a compound of formula II, III, IV, V, VI, VII, VIII, or IX:

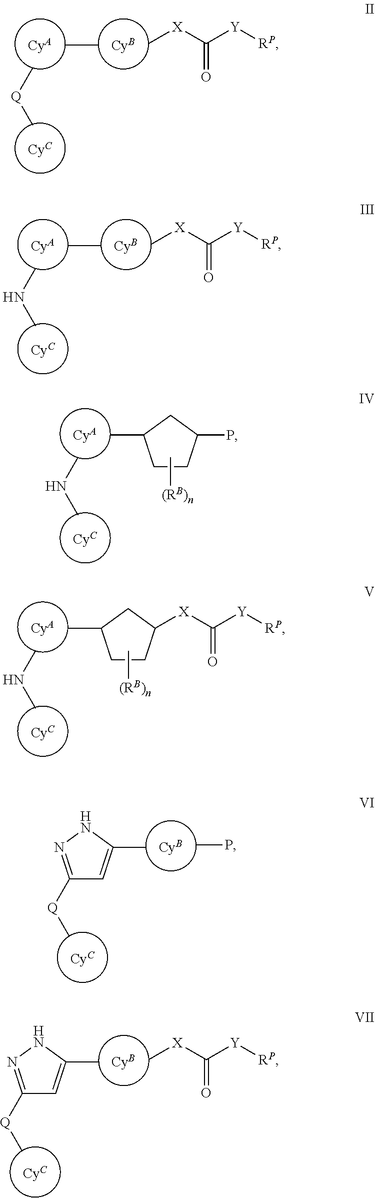

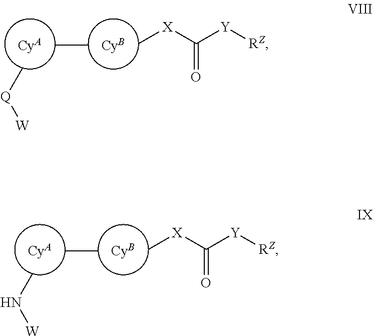

or a pharmaceutically acceptable salt thereof, wherein each of Cy^{A}, Cy^{B}, Cy^{C}, Q, P, W, X, Y, R^{Z}, R^{B}, and n is as defined in embodiments and classes and subclasses herein.

In some embodiments, the present disclosure provides a compound of formula X-a, X-b, X-c, XI-a, XI-b, XI-c, XII-a, XII-b, XII-c, XIII-a, XIII-b, XIII-c, XIV-a, XIV-b, XIV-c, XV-a, XV-b, XV-c, XVI-a, XVI-b, XVI-c, XVII-a, XVII-b, XVII-c, XVIII-a, XVIII-b, XVIII-c, XIX-a, XIX-b, XIX-c, XX-a, XX-b, XX-c, XXI-a, XXI-b, XXI-c, XXII-a, XXII-b, XXII-c, XXIII-a, XXIII-b, XXIII-c, XXIV-a, XXIV-b, XXIV-c, XXV-a, XXV-b, or XXV-c:

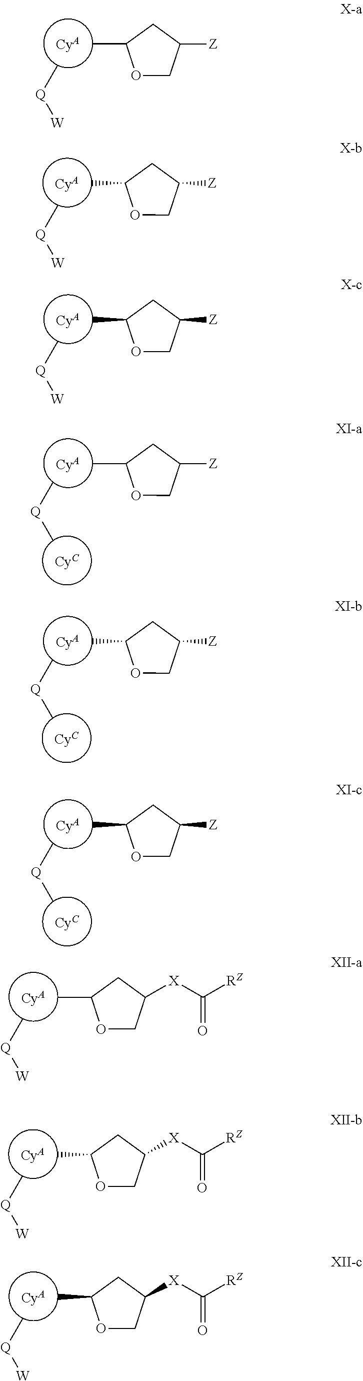

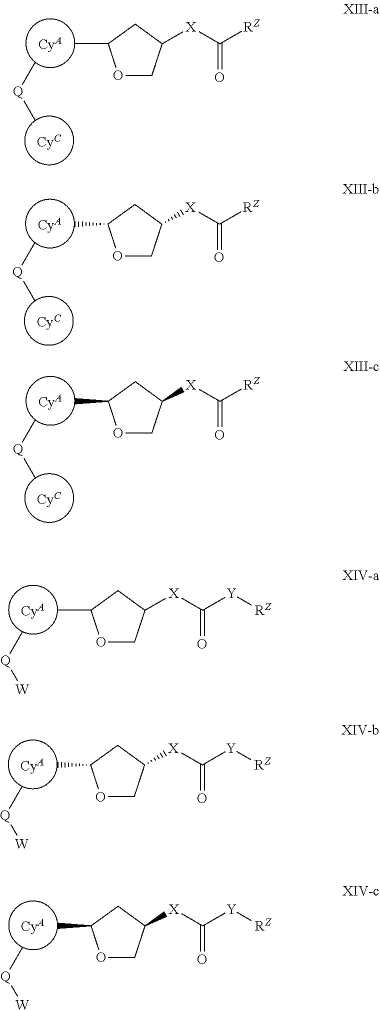

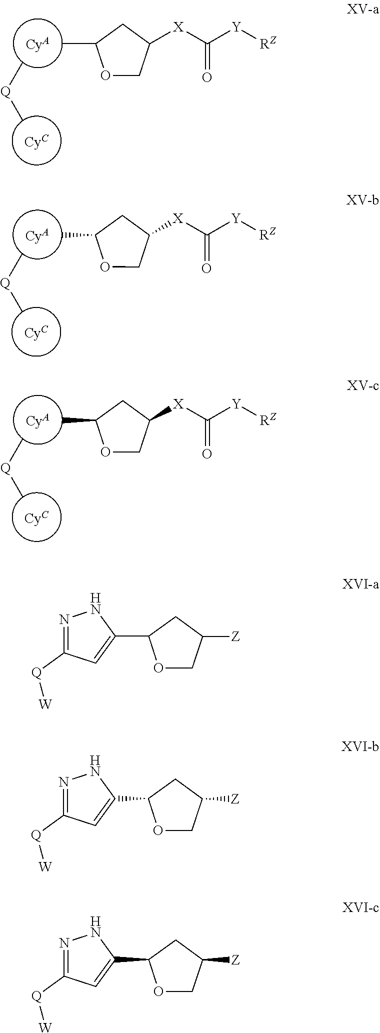

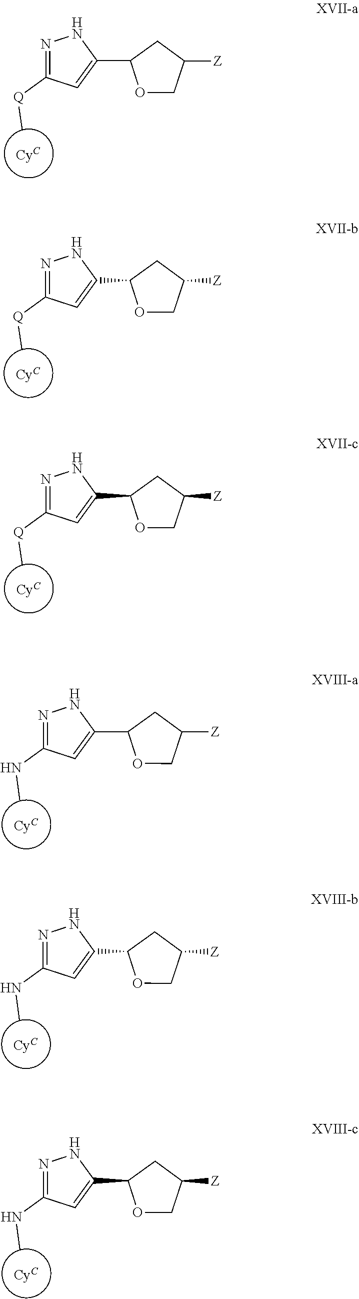

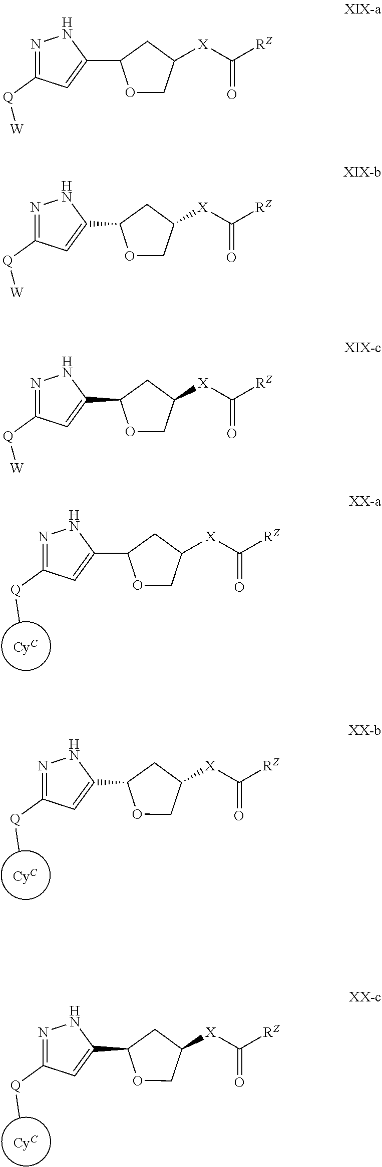

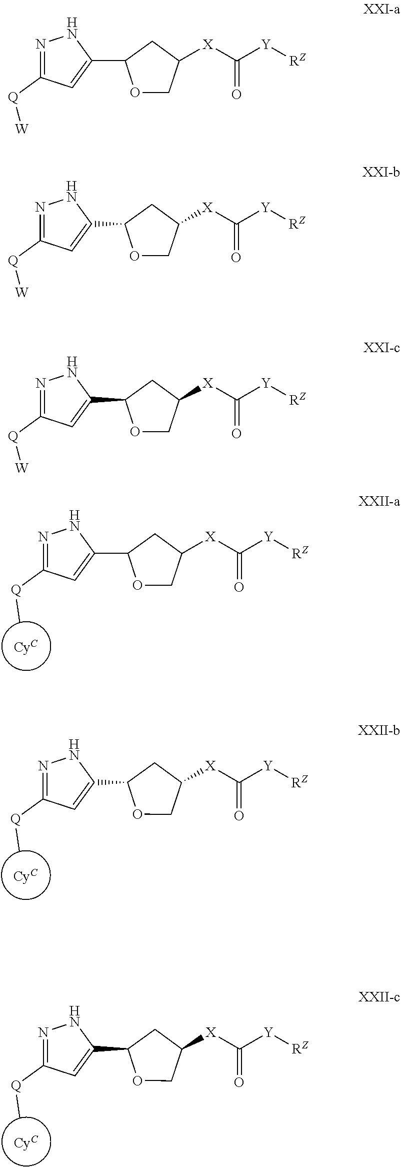

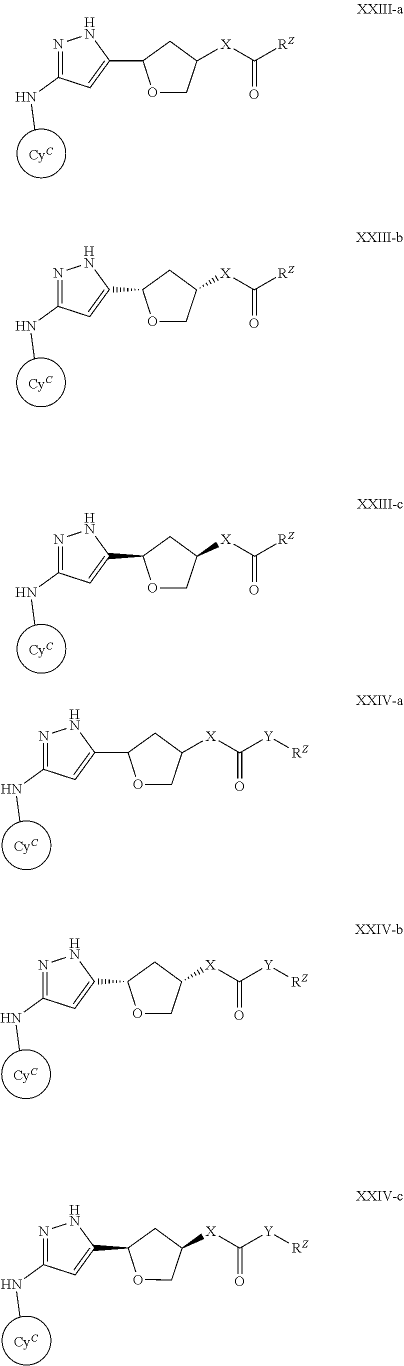

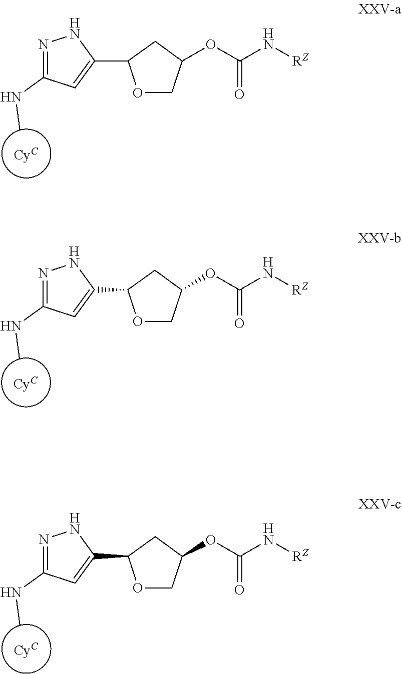

or a pharmaceutically acceptable salt thereof, wherein each of Cy^{A}, C^{C}, Q, Z, W, X, Y, and R^{Z} is as defined in embodiments and classes and subclasses herein.

In some embodiments, the present disclosure provides a compound of formula XXVI-a, XXVI-b, XXVI-c, XXVII-a, XXVII-b, XXVII-c, XXVIII-a, XXVIII-b, or XXVIII-c:

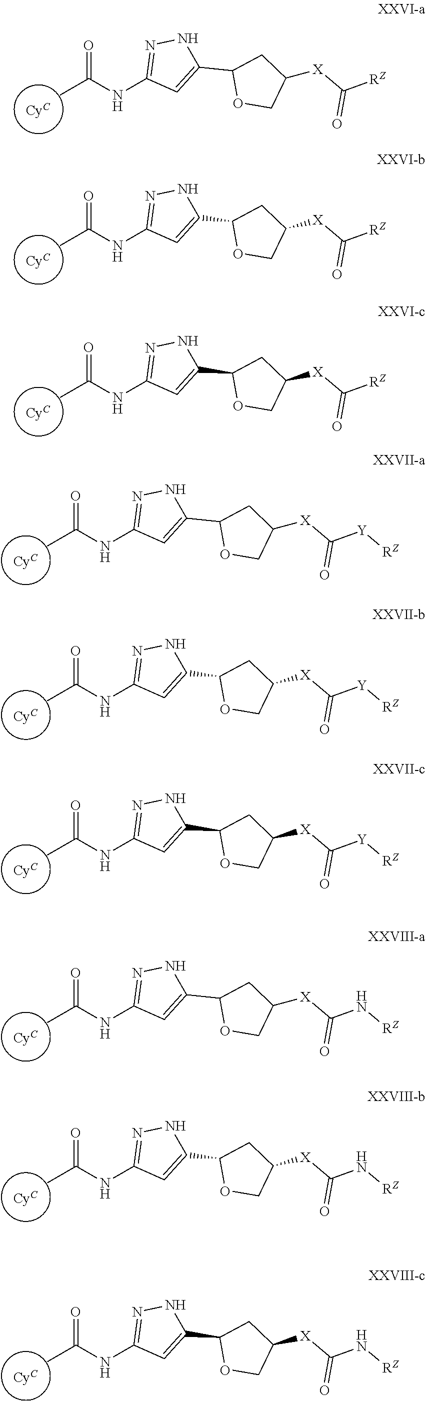

or a pharmaceutically acceptable salt thereof, wherein each of Cy^{C}, X, Y, and R^{Z} is as defined in embodiments and classes and subclasses herein.

In some embodiments, the present disclosure provides a pharmaceutical composition comprising a compound of formula I, or a pharmaceutically acceptable salt thereof, and a pharmaceutically acceptable carrier, adjuvant, or diluent. In some embodiments, the present disclosure provides a pharmaceutical composition comprising a compound of the disclosure, for example, a compound of formula II, III, IV, V, VI, VII, VIII, IX, X-a, X-b, X-c, XI-a, XI-b, XI-c, XII-a, XII-b, XII-c, XIII-a, XIII-b, XIII-c, XIV-a, XIV-b, XIV-c, XV-a, XV-b, XV-c, XVI-a, XVI-b, XVI-c, XVII-a, XVII-b, XVII-c, XVIII-a, XVIII-b, XVIII-c, XIX-a, XIX-b, XIX-c, XX-a, XX-b, XX-c, XXI-a, XXI-b, XXI-c, XXII-a, XXII-b, XXII-c, XXIII-a, XXIII-b, XXIII-c, XXIV-a, XXIV-b, XXIV-c, XXV-a, XXV-b, XXV-c, XXVI-a, XXVI-b, XXVI-c, XXVII-a, XXVII-b, XXVII-c, XXVIII-a, XXVIII-b, or XXVIII-c or a pharmaceutically acceptable salt thereof, and a pharmaceutically acceptable carrier, adjuvant, or diluent. In some embodiments, the present disclosure provides a pharmaceutical composition comprising a compound of the disclosure, for example, a compound of formula I-A, or a pharmaceutically acceptable salt thereof, and a pharmaceutically acceptable carrier, adjuvant, or diluent.

In some embodiments, the present disclosure provides a method of treating a CDK2-mediated disorder comprising administering to a patient in need thereof a compound of formula I, or composition comprising said compound. In some embodiments, the present disclosure provides a method of treating a CDK2-mediated disorder comprising administering to a patient in need thereof a compound of the disclosure, for example, a compound of formula II, III, IV, V, VI, VII, VIII, IX, X-a, X-b, X-c, XI-a, XI-b, XI-c, XII-a, XII-b, XII-c, XIII-a, XIII-b, XIII-c, XIV-a, XIV-b, XIV-c, XV-a, XV-b, XV-c, XVI-a, XVI-b, XVI-c, XVII-a, XVII-b, XVII-c, XVIII-a, XVIII-b, XVIII-c, XIX-a, XIX-b, XIX-c, XX-a, XX-b, XX-c, XXI-a, XXI-b, XXI-c, XXII-a, XXII-b, XXII-c, XXIII-a, XXIII-b, XXIII-c, XXIV-a, XXIV-b, XXIV-c, XXV-a, XXV-b, XXV-c, XXVI-a, XXVI-b, XXVI-c, XXVII-a, XXVII-b, XXVII-c, XXVIII-a, XXVIII-b, or XXVIII-c or composition comprising said compound. In some embodiments, the present disclosure provides a method of treating a CDK2-mediated disorder comprising administering to a patient in need thereof a compound of the disclosure, for example, a compound of formula I-A, or composition comprising said compound.

In some embodiments, the present disclosure provides a process for providing a compound of formula I, or synthetic intermediates thereof. In some embodiments, the present disclosure provides a process for providing a compound of the disclosure, for example, a compound of formula II, III, IV, V, VI, VII, VIII, IX, X-a, X-b, X-c, XI-a, XI-b, XI-c, XII-a, XII-b, XII-c, XIII-a, XIII-b, XIII-c, XIV-a, XIV-b, XIV-c, XV-a, XV-b, XV-c, XVI-a, XVI-b, XVI-c, XVII-a, XVII-b, XVII-c, XVIII-a, XVIII-b, XVIII-c, XIX-a, XIX-b, XIX-c, XX-a, XX-b, XX-c, XXI-a, XXI-b, XXI-c, XXII-a, XXII-b, XXII-c, XXIII-a, XXIII-b, XXIII-c, XXIV-a, XXIV-b, XXIV-c, XXV-a, XXV-b, XXV-c, XXVI-a, XXVI-b, XXVI-c, XXVII-a, XXVII-b, XXVII-c, XXVIII-a, XXVIII-b, or XXVIII-c or synthetic intermediates thereof. In some embodiments, the present disclosure provides a process for providing a compound of the disclosure, for example, a compound of formula I-A, or synthetic intermediates thereof.

In some embodiments, the present disclosure provides a process for providing pharmaceutical compositions comprising compounds of formula I. In some embodiments, the present disclosure provides a process for providing pharmaceutical compositions comprising compounds of the disclosure, for example, a compound of formula II, III, IV, V, VI, VII, VIII, IX, X-a, X-b, X-c, XI-a, XI-b, XI-c, XII-a, XII-b, XII-c, XIII-a, XIII-b, XIII-c, XIV-a, XIV-b, XIV-c, XV-a, XV-b, XV-c, XVI-a, XVI-b, XVI-c, XVII-a, XVII-b, XVII-c, XVIII-a, XVIII-b, XVIII-c, XIX-a, XIX-b, XIX-c, XX-a, XX-b, XX-c, XXI-a, XXI-b, XXI-c, XXII-a, XXII-b, XXII-c, XXIII-a, XXIII-b, XXIII-c, XXIV-a, XXIV-b, XXIV-c, XXV-a, XXV-b, XXV-c, XXVI-a, XXVI-b, XXVI-c, XXVII-a, XXVII-b, XXVII-c, XXVIII-a, XXVIII-b, or XXVIII-c. In some embodiments, the present disclosure provides a process for providing pharmaceutical compositions comprising compounds of the disclosure, for example, a compound of formula I-A.

### DETAILED DESCRIPTION

### 1. General Description of Certain Embodiments

Compounds provided herein, and pharmaceutical compositions thereof, are useful as inhibitors of CDK2. In some embodiments, the present disclosure provides a compound of formula I-A:

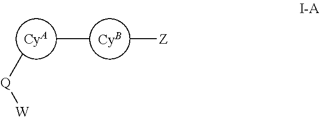

or a pharmaceutically acceptable salt thereof, wherein:
Q is L¹;
Cy^{A} is a 5-6 membered heteroarylene having 1-4 heteroatoms independently selected from nitrogen, oxygen, and sulfur; wherein Cy^{A} is substituted with m instances of R^{A} in addition to Q and Cy^{B};
Cy^{B} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenylene; or a 5-14 membered heteroarylene having 1-4 heteroatoms independently selected from nitrogen, oxygen, and sulfur; wherein Cy^{B} is substituted with n instances of R^{B} in addition to Cy^{A} and Z;
Z is hydrogen or L²-R^{Z};
R^{Z} is hydrogen, or an optionally substituted group selected from C₁₋₈ aliphatic, a saturated or partially unsaturated 3-14 membered carbocyclic ring, phenyl, a 3-10 membered saturated or partially unsaturated heterocyclic ring having 1-4 heteroatoms independently selected from nitrogen, oxygen, and sulfur, and a 5-14 membered heteroaryl ring having 1-4 heteroatoms independently selected from nitrogen, oxygen, and sulfur;
W is hydrogen or Cy^{C};
Cy^{C} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenyl; or a 5-14 membered heteroaryl having 1-4 heteroatoms independently selected from nitrogen, oxygen, and sulfur; wherein Cy^{C} is substituted with p instances of R^{C} in addition to Q;
each instance of R^{A}, R^{B}, and R^{C} is independently R¹ or R², wherein R^{A} is substituted by q^{A} instances of R³, R^{B} is substituted by q^{B} instances of R³, and R^{C} is substituted by q^{C} instances of R³; or
two instances of R^{A}, two instances of R^{B}, two instances of R^{C}, an instance of R^{A} and an instance of R^{L}, or an instance of R^{C} and an instance of R^{L} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₅ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein
each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R¹ is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R;
each instance of R² is independently C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of m, n, p, q^{A}, q^{B}, q^{C}, r, and t is independently 0, 1, 2, 3, or 4.

In some embodiments, the present disclosure provides a compound of formula I:

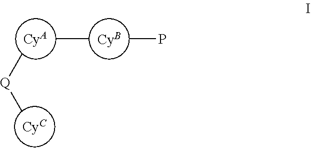

or a pharmaceutically acceptable salt thereof, wherein:
   Q is L¹;
Cy^{A} is a 5-6 membered heteroarylene having 1-4 heteroatoms independently selected from nitrogen, oxygen, and sulfur; wherein Cy^{A} is substituted with m instances of R^{A} in addition to Q and Cy^{B};
Cy^{B} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenylene; or a 5-14 membered heteroarylene having 1-4 heteroatoms independently selected from nitrogen, oxygen, and sulfur; wherein Cy^{B} is substituted with n instances of R^{B} in addition to Cy^{A} and P;
P is hydrogen or —L²-R^{P};
R^{P} is R;
Cy^{C} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenyl; or a 5-14 membered heteroaryl having 1-4 heteroatoms independently selected from nitrogen, oxygen, and sulfur; wherein Cy^{C} is substituted with p instances of R^{C} in addition to Q;
each instance of R^{A}, R^{B}, and R^{C} is independently R¹ or R², wherein R^{A} is substituted by q^{A} instances of R³, R^{B} is substituted by q^{B} instances of R³, and R^{C} is substituted by q^{C} instances of R³; or
two instances of R^{A}, two instances of R^{B}, two instances of R^{C}, an instance of R^{A} and an instance of R^{L}, or an instance of R^{C} and an instance of R^{L} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₅ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein
each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R¹ is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R;
each instance of R² is independently C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each of m, n, p, q^{A}, q^{B}, q^{C}, r, and t is independently 0, 1, 2, 3, or 4.

In some embodiments, the present disclosure provides a compound of formula II, III, IV, V, VI, VII, VIII, or IX:

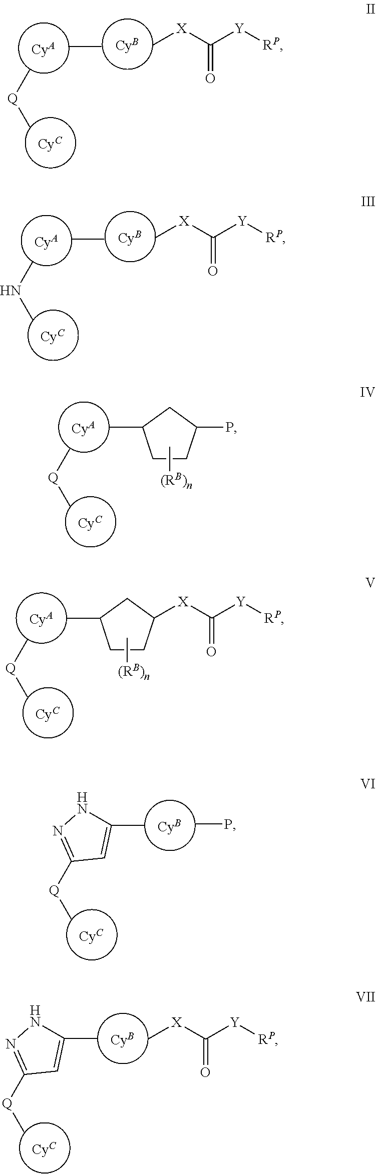

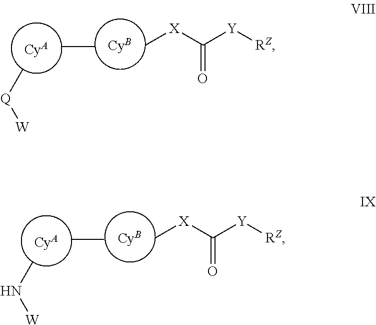

or a pharmaceutically acceptable salt thereof, wherein:
   Q is L¹;
Cy^{A} is a 5-6 membered heteroarylene having 1-4 heteroatoms independently selected from nitrogen, oxygen, and sulfur; wherein Cy^{A} is substituted with m instances of R^{A} in addition to Q and Cy^{B};
Cy^{B} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenylene; or a 5-14 membered heteroarylene having 1-4 heteroatoms independently selected from nitrogen, oxygen, and sulfur; wherein Cy^{B} is substituted with n instances of R^{B} in addition to Cy^{A} and P or X;
X is selected from O, NR^{X}, and S;
Y is selected from O, NR^{Y}, and S;
each instance of R^{X} and R^{Y} is independently R;
R^{Z} is hydrogen, or an optionally substituted group selected from C₁₋₈ aliphatic, a saturated or partially unsaturated 3-14 membered carbocyclic ring, phenyl, a 3-10 membered saturated or partially unsaturated heterocyclic ring having 1-4 heteroatoms independently selected from nitrogen, oxygen, and sulfur, and a 5-14 membered heteroaryl ring having 1-4 heteroatoms independently selected from nitrogen, oxygen, and sulfur;
P is hydrogen or —L²-R^{P};
R^{P} is R;
W is hydrogen or Cy^{C};
Cy^{C} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenyl, or a 5-14 membered heteroaryl having 1-4 heteroatoms independently selected from nitrogen, oxygen, and sulfur; wherein Cy^{C} is substituted with p instances of R^{C} in addition to Q;
each instance of R^{A}, R^{B}, and R^{C} is independently R¹ or R², wherein R^{A} is substituted by q^{A} instances of R³, R^{B} is substituted by q^{B} instances of R³, and R^{C} is substituted by q^{C} instances of R³; or
two instances of R^{A}, two instances of R^{B}, two instances of R^{C}, an instance of R^{A} and an instance of R^{L}, or an instance of R^{C} and an instance of R^{L} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₅ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R¹ is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R;
each instance of R² is independently C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each of m, n, p, q^{A}, q^{B}, q^{C}, r, and t is independently 0, 1, 2, 3, or 4.

In some embodiments, the present disclosure provides a compound of formula X-a, X-b, X-c, XI-a, XI-b, XI-c, XII-a, XII-b, XII-c, XIII-a, XIII-b, XIII-c, XIV-a, XIV-b, XIV-c, XV-a, XV-b, XV-c, XVI-a, XVI-b, XVI-c, XVII-a, XVII-b, XVII-c, XVIII-a, XVIII-b, XVIII-c, XIX-a, XIX-b, XIX-c, XX-a, XX-b, XX-c, XXI-a, XXI-b, XXI-c, XXII-a, XXII-b, XXII-c, XXIII-a, XXIII-b, XXIII-c, XXIV-a, XXIV-b, XXIV-c, XXV-a, XXV-b, or XXV-c:

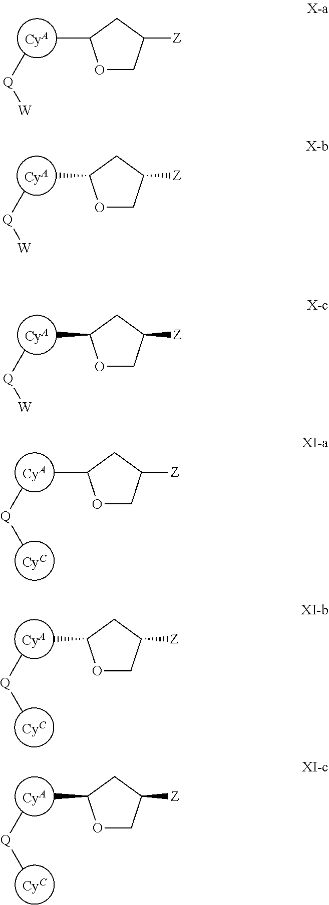

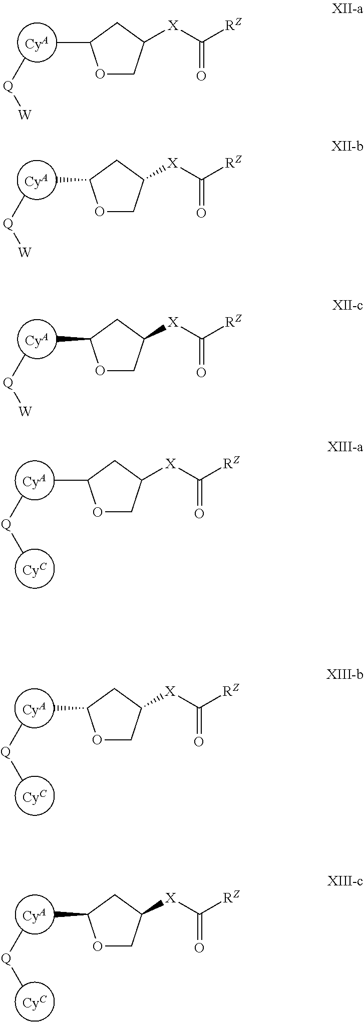

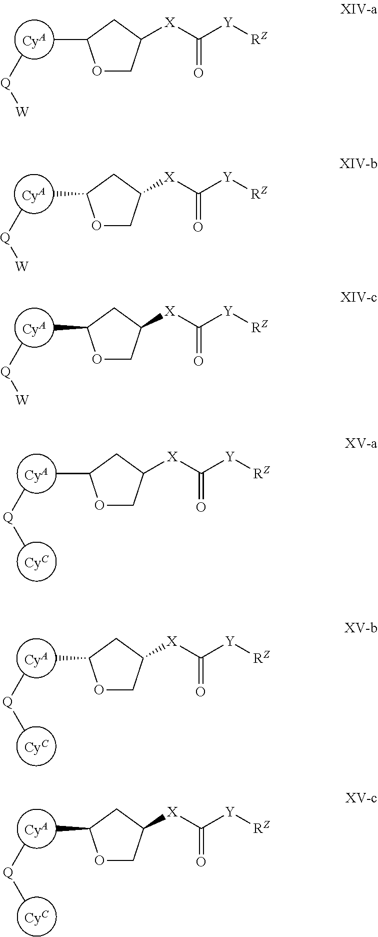

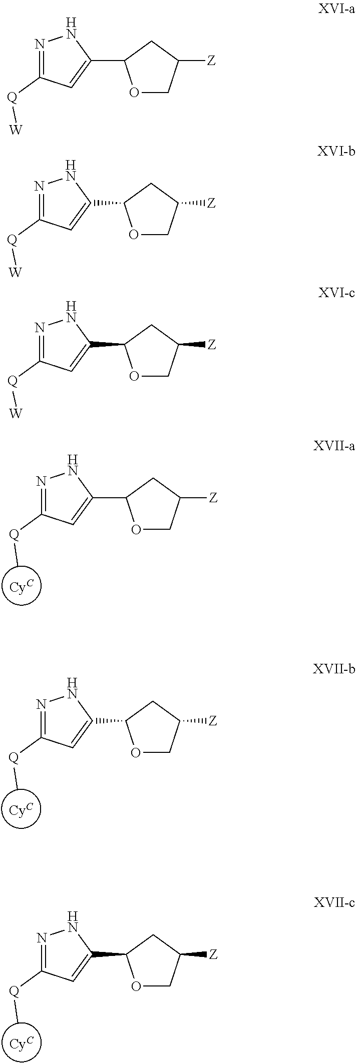

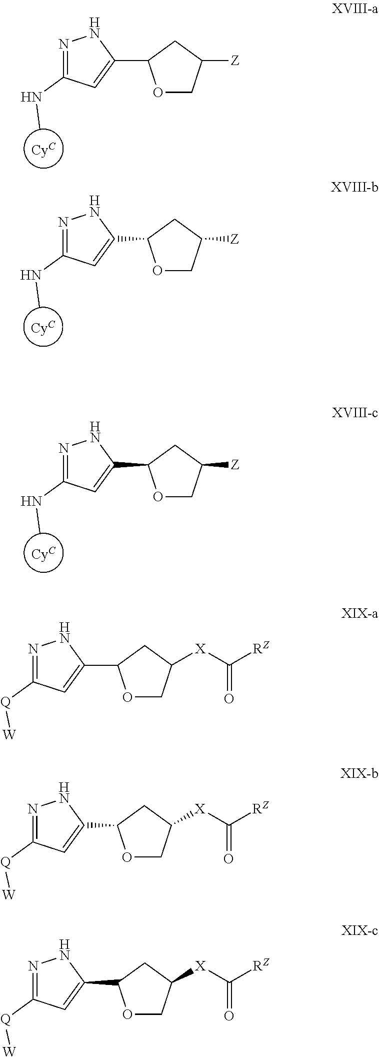

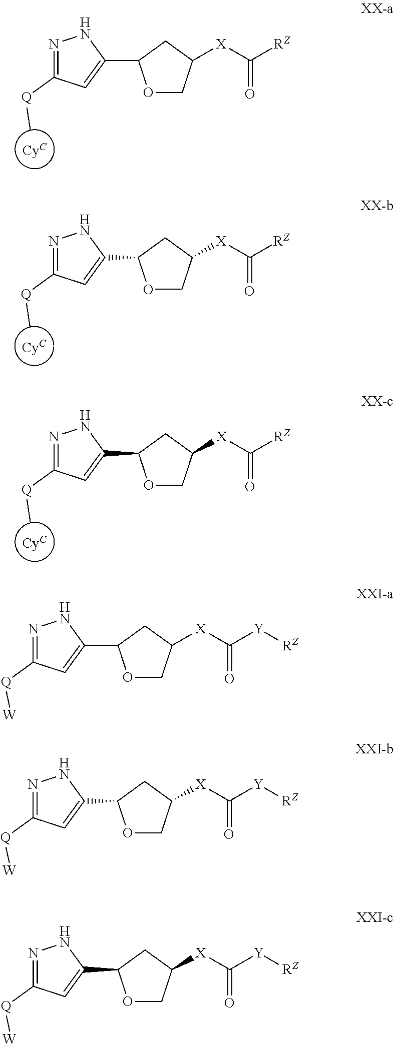

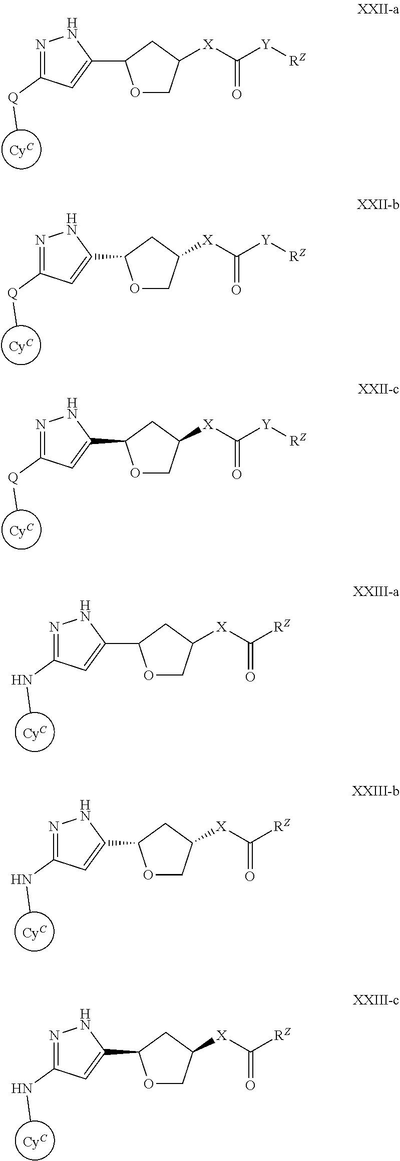

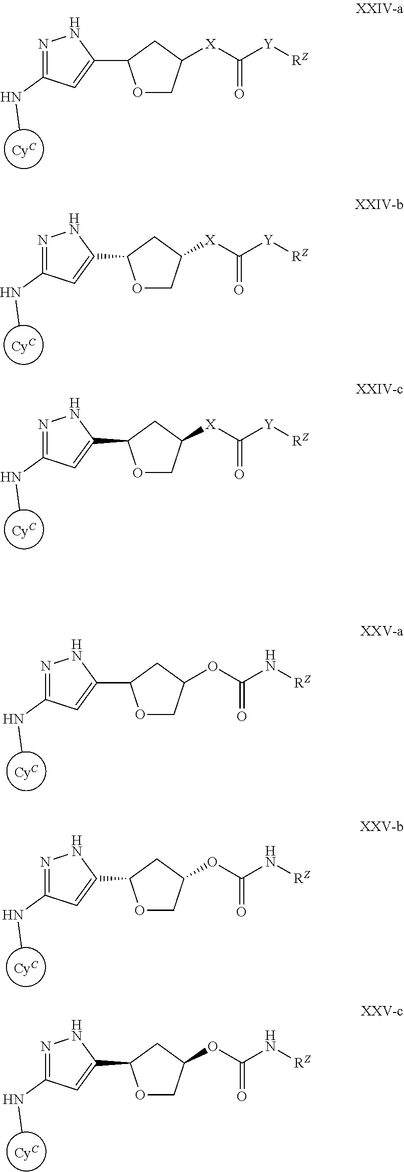

or a pharmaceutically acceptable salt thereof, wherein each of Cy^{A}, C^{C}, Q, Z, W, X, Y, and R^{Z} is as defined in embodiments and classes and subclasses herein.

In some embodiments, the present disclosure provides a compound of formula XXVI-a, XXVI-b, XXVI-c, XXVII-a, XXVII-b, XXVII-c, XXVIII-a, XXVIII-b, or XXVIII-c:

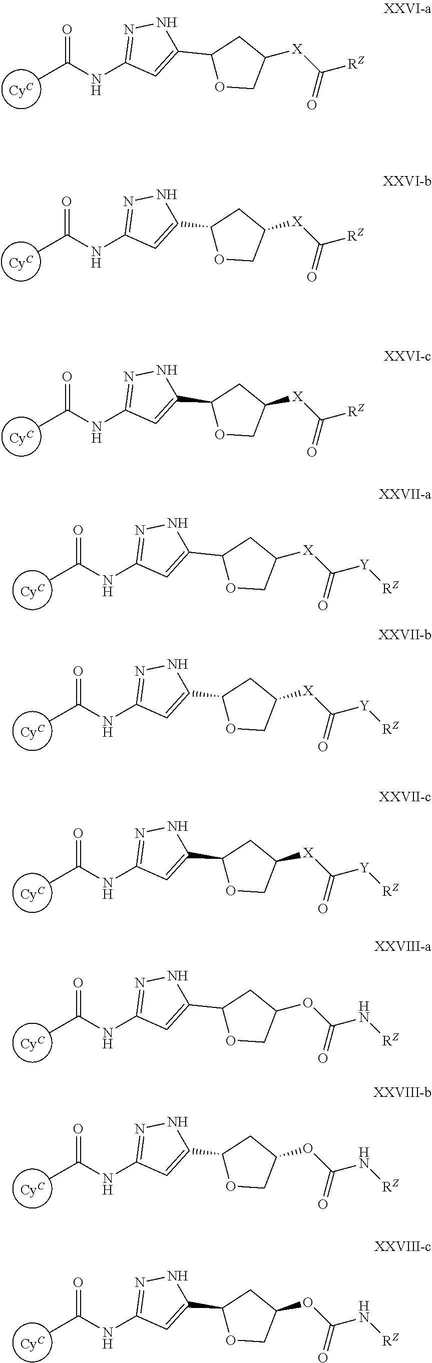

or a pharmaceutically acceptable salt thereof, wherein each of Cy^{C}, X, Y, and R^{Z} is as defined in embodiments and classes and subclasses herein.

### 2. Compounds and Definitions

Compounds described herein include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, ₇₅^{th} Ed. Additionally, general principles of organic chemistry are described in “Organic Chemistry”, Thomas Sorrell, University Science Books, Sausalito: 1999, and “March's Advanced Organic Chemistry”, ₅^{th} Ed., Ed.: Smith, M. B. and March, J., John Wiley & Sons, New York: 2001, the entire contents of which are hereby incorporated by reference. Chemical names, common names, and chemical structures may be used interchangeably to describe the same structure. If a chemical compound is referred to using both a chemical structure and a chemical name, and an ambiguity exists between the structure and the name, the structure predominates.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. A carbocycle can be, under certain circumstances, a bridged bicyclic or a fused ring such as, e.g., an ortho-fused carbocycle, a spirofused carbocycle, etc. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl”, unless otherwise indicated, as used herein, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl,” used alone or as part of a larger moiety, refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of compounds described herein, “aryl” refers to an aromatic ring system which includes, but is not limited to, phenyl, naphthyl, anthracyl and the like, which may bear one or more substituents. It will be appreciated that an “aryl” group can comprise carbon and heteroatom ring members.

The terms “heteroaryl” or “heteroaromatic”, unless otherwise defined, as used herein refers to a monocyclic aromatic 5-6 membered ring containing one or more heteroatoms, for example one to four heteroatoms, such as nitrogen, oxygen, and sulfur, or an 8-10 membered polycyclic ring system containing one or more heteroatoms, wherein at least one ring in the polycyclic ring system is aromatic, and the point of attachment of the polycyclic ring system is through a ring atom on an aromatic ring. A heteroaryl ring may be linked to adjacent radicals though carbon or nitrogen. Examples of heteroaryl rings include but are not limited to furan, thiophene, pyrrole, thiazole, oxazole, isothiazole, isoxazole, imidazole, pyrazole, triazole, pyridine, pyrimidine, indole, etc. For example, unless otherwise defined, 1,2,3,4-tetrahydroquinoline is a heteroaryl ring if its point of attachment is through the benzo ring, e.g.:

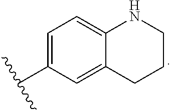 [SMILES: *c1ccc2c(c1)CCCN2]

The terms “heterocyclyl” or “heterocyclic group”, unless otherwise defined, refer to a saturated or partially unsaturated 3-10 membered monocyclic or 7-14 membered polycyclic ring system, including bridged or fused rings (e.g., an ortho-fused bicyclic or a spirofused bicyclic ring), and whose ring system includes one to four heteroatoms, such as nitrogen, oxygen, and sulfur. A heterocyclyl ring may be linked to adjacent radicals through carbon or nitrogen.

The term “partially unsaturated” in the context of rings, unless otherwise defined, refers to a monocyclic ring, or a component ring within a polycyclic (e.g. bicyclic, tricyclic, etc.) ring system, wherein the component ring contains at least one degree of unsaturation in addition to those provided by the ring itself, but is not aromatic. Examples of partially unsaturated rings include, but are not limited to, 3,4-dihydro-2H-pyran, 3-pyrroline, 2-thiazoline, etc. Where a partially unsaturated ring is part of a polycyclic ring system, the other component rings in the polycyclic ring system may be saturated, partially unsaturated, or aromatic, but the point of attachment of the polycyclic ring system is on a partially unsaturated component ring. For example, unless otherwise defined, 1,2,3,4-tetrahydroquinoline is a partially unsaturated ring if its point of attachment is through the piperidino ring, e.g.:

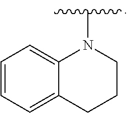 [SMILES: *N1CCCc2ccccc21]

The term “saturated” in the context of rings, unless otherwise defined, refers to a 3-10 membered monocyclic ring, or a 7-14 membered polycyclic (e.g. bicyclic, tricyclic, etc.) ring system, wherein the monocyclic ring or the component ring that is the point of attachment for the polycyclic ring system contains no additional degrees of unsaturation in addition to that provided by the ring itself. Examples of monocyclic saturated rings include, but are not limited to, azetidine, oxetane, cyclohexane, etc. Where a saturated ring is part of a polycyclic ring system, the other component rings in the polycyclic ring system may be saturated, partially unsaturated, or aromatic, but the point of attachment of the polycyclic ring system is on a saturated component ring. For example, unless otherwise defined, 2-azaspiro[3.4]oct-6-ene is a saturated ring if its point of attachment is through the azetidino ring, e.g.:

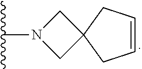 [SMILES: *N1CC2(CC=CC2)C1]

The terms “alkylene”, “arylene”, “cycloalkylene”, “heteroarylene”, “heterocycloalkylene”, and the other similar terms with the suffix “-ylene” as used herein refers to a divalently bonded version of the group that the suffix modifies. For example, “alkylene” is a divalent alkyl group connecting the groups to which it is attached.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups.

Additionally or alternatively, any substitutable nitrogen of a bridged bicyclic group is optionally substituted. Exemplary bridged bicyclics include:

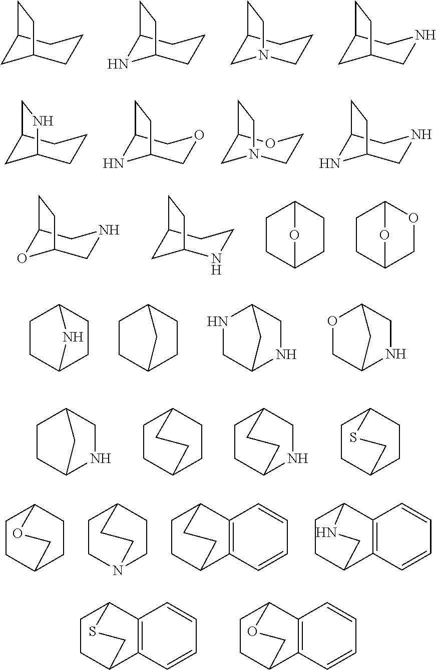

As described herein, compounds described herein may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)(OR^{∘})R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋2R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR′2, —NO₂, —SiR^{•}3, —OSiR^{•}3, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂-3S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NRW^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NRT₂, —C(NH)NRT₂, or —N(RT)S(O)₂R; wherein each R^{†}is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “isomer” as used herein refers to a compound having the identical chemical formula but different structural or optical configurations. The term “stereoisomer” as used herein refers to and includes isomeric molecules that have the same molecular formula but differ in positioning of atoms and/or functional groups in the space. All stereoisomers of the present compounds (e.g., those which may exist due to asymmetric carbons on various substituents), including enantiomeric forms and diastereomeric forms, are contemplated within the scope of this disclosure. Therefore, unless otherwise stated, single stereochemical isomers as well as mixtures of enantiomeric, diastereomeric, and geometric (or conformational) isomers of the present compounds are within the scope of the disclosure.

The term “tautomer” as used herein refers to one of two or more structural isomers which exist in equilibrium and which are readily converted from one isomeric form to another. It is understood that tautomers encompass valence tautomers and proton tautomers (also known as prototropic tautomers). Valence tautomers include interconversions by reorganization of some of the bonding electrons. Proton tautomers include interconversions via migration of a proton, such as keto-enol and imine-enamine isomerizations. Unless otherwise stated, all tautomers of the compounds described herein are within the scope of the disclosure.

The term “isotopic substitution” as used herein refers to the substitution of an atom with its isotope. The term “isotope” as used herein refers to an atom having the same atomic number as that of atoms dominant in nature but having a mass number (neutron number) different from the mass number of the atoms dominant in nature. It is understood that a compound with an isotopic substitution refers to a compound in which at least one atom contained therein is substituted with its isotope. Atoms that can be substituted with its isotope include, but are not limited to, hydrogen, carbon, and oxygen. Examples of the isotope of a hydrogen atom include ²H (also represented as D) and ³H. Examples of the isotope of a carbon atom include ¹³C and ¹⁴C. Examples of the isotope of an oxygen atom include ¹⁸O. Unless otherwise stated, all isotopic substitution of the compounds described herein are within the scope of the disclosure. Such compounds are useful, for example, as analytical tools, as probes in biological assays, or as therapeutic agents in accordance with the present disclosure.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Exemplary pharmaceutically acceptable salts are found, e.g., in Berge, et al. (J. Pharm. Sci. 1977, 66(1), 1; and Gould, P. L., Int. J. Pharmaceutics 1986, 33, 201-217; (each hereby incorporated by reference in its entirety).

Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Pharmaceutically acceptable salts are also intended to encompass hemi-salts, wherein the ratio of compound:acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient, when administered to a subject or population suffering from or susceptible to a disease, disorder, and/or condition in accordance with a therapeutic dosing regimen, to treat (e.g., effect beneficial or desired results, including clinical results) the disease, disorder, and/or condition. As such, the effective amount may be sufficient, e.g., to reduce or ameliorate the severity and/or duration of afflictions related to CDK2 signaling, or one or more symptoms thereof, prevent the advancement of conditions or symptoms related to afflictions related to CDK2 signaling, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable. “Treatment” can also mean prolonging survival as compared to expected survival if not receiving treatment. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “in need thereof” refers to the need for symptomatic or asymptomatic relief from conditions related to CDK2 signaling activity or that may otherwise be relieved by the compounds and/or compositions of the disclosure.

### 3. Description of Exemplary Embodiments

In some embodiments, the present disclosure provides a compound of formula I-A:

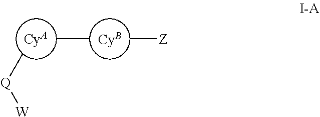

or a pharmaceutically acceptable salt thereof, wherein:
Q is L¹;
Cy^{A} is a 5-6 membered heteroarylene having 1-4 heteroatoms independently selected from nitrogen, oxygen, and sulfur; wherein Cy^{A} is substituted with m instances of R^{A} in addition to Q and Cy^{B};
Cy^{B} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenylene; or a 5-14 membered heteroarylene having 1-4 heteroatoms independently selected from nitrogen, oxygen, and sulfur; wherein Cy^{B} is substituted with n instances of R^{B} in addition to Cy^{A} and Z;
Z is hydrogen or L²-R^{Z};
R^{Z} is hydrogen, or an optionally substituted group selected from C₁₋₈ aliphatic, a saturated or partially unsaturated 3-14 membered carbocyclic ring, phenyl, a 3-10 membered saturated or partially unsaturated heterocyclic ring having 1-4 heteroatoms independently selected from nitrogen, oxygen, and sulfur, and a 5-14 membered heteroaryl ring having 1-4 heteroatoms independently selected from nitrogen, oxygen, and sulfur;
W is hydrogen or Cy^{C};
Cy^{C} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenyl; or a 5-14 membered heteroaryl having 1-4 heteroatoms independently selected from nitrogen, oxygen, and sulfur; wherein Cy^{C} is substituted with p instances of R^{C} in addition to Q;
each instance of R^{A}, R^{B}, and R^{C} is independently R¹ or R², wherein R^{A} is substituted by q^{A} instances of R³, R^{B} is substituted by q^{B} instances of R³, and R^{C} is substituted by q^{C} instances of R³; or
two instances of R^{A}, two instances of R^{B}, two instances of R^{C}, an instance of R^{A} and an instance of R^{L}, or an instance of R^{C} and an instance of R^{L} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₅ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R¹ is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R;
each instance of R² is independently C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of m, n, p, q^{A} B q^{C} q^{Z}, r, and t is independently 0, 1, 2, 3, or 4.

As described above, in some embodiments, the present disclosure provides a compound of formula I:

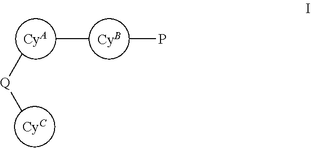

or a pharmaceutically acceptable salt thereof, wherein:
Q is L¹;
Cy^{A} is a 5-6 membered heteroarylene having 1-4 heteroatoms independently selected from nitrogen, oxygen, and sulfur; wherein Cy^{A} is substituted with m instances of R^{A} in addition to Q and Cy^{B};
Cy^{B} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenylene; or a 5-14 membered heteroarylene having 1-4 heteroatoms independently selected from nitrogen, oxygen, and sulfur; wherein Cy^{B} is substituted with n instances of R^{B} in addition to Cy^{A} and P;
P is hydrogen or —L²-R^{P};
R^{P} is R;
Cy^{C} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenyl; or a 5-14 membered heteroaryl having 1-4 heteroatoms independently selected from nitrogen, oxygen, and sulfur; wherein Cy^{C} is substituted with p instances of R^{C} in addition to Q;
each instance of R^{A}, R^{B}, and R^{C} is independently R¹ or R², wherein R^{A} is substituted by q^{A} instances of R³, R is substituted by q^{B} instances of R³, and R^{C} is substituted by q^{C} instances of R³; or
two instances of R^{A}, two instances of R^{B}, two instances of R^{C}, an instance of R^{A} and an instance of R^{L}, or an instance of R^{C} and an instance of R^{L} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₅ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein
each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R¹ is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R;
each instance of R² is independently C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each of m, n, p, q^{A}, q^{B}, q^{C}, r, and t is independently 0, 1, 2, 3, or 4.

As described above, in some embodiments, the present disclosure provides a compound of formula II, III, IV, V, VI, or VII:

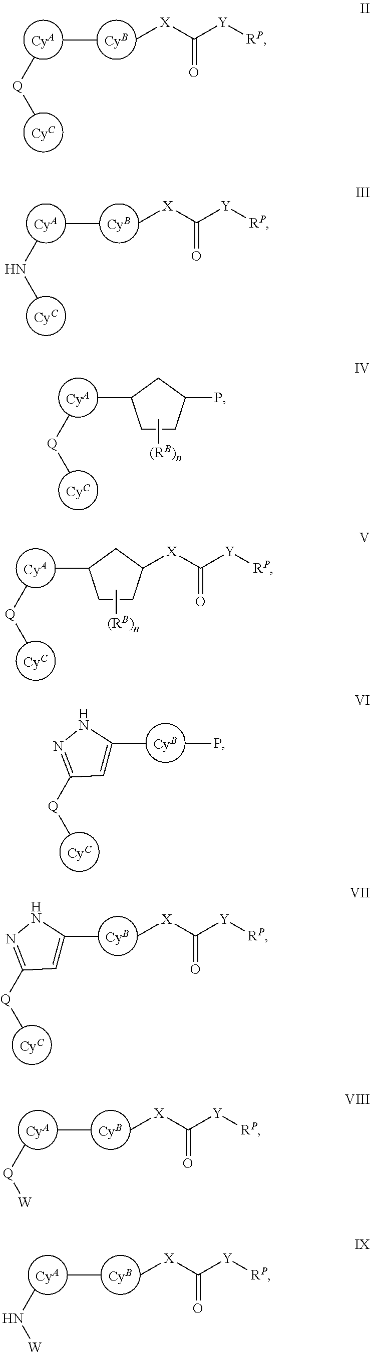

or a pharmaceutically acceptable salt thereof, wherein:
Q is L¹;
Cy^{A} is a 5-6 membered heteroarylene having 1-4 heteroatoms independently selected from nitrogen, oxygen, and sulfur; wherein Cy^{A} is substituted with m instances of R^{A} in addition to Q and Cy^{B};
Cy^{B} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenylene; or a 5-14 membered heteroarylene having 1-4 heteroatoms independently selected from nitrogen, oxygen, and sulfur; wherein Cy^{B} is substituted with n instances of R^{B} in addition to Cy^{A} and P or X;
X is selected from O, NR^{X}, and S;
Y is selected from O, NR^{Y}, and S;
each instance of R^{X} and R^{Y} is independently R;
R^{Z} is hydrogen, or an optionally substituted group selected from C₁₋₈ aliphatic, a saturated or partially unsaturated 3-14 membered carbocyclic ring, phenyl, a 3-10 membered saturated or partially unsaturated heterocyclic ring having 1-4 heteroatoms independently selected from nitrogen, oxygen, and sulfur, and a 5-14 membered heteroaryl ring having 1-4 heteroatoms independently selected from nitrogen, oxygen, and sulfur;
P is hydrogen or —L²-R^{P};
R^{P} is R;
W is hydrogen or Cy^{C};
Cy^{C} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenyl, or a 5-14 membered heteroaryl having 1-4 heteroatoms independently selected from nitrogen, oxygen, and sulfur; wherein Cy^{C} is substituted with p instances of R^{C} in addition to Q;
each instance of R^{A}, R^{B}, and R^{C} is independently R¹ or R², wherein R^{A} is substituted by q^{A} instances of R³, R^{B} is substituted by q^{B} instances of R³, and R^{C} is substituted by q^{C} instances of R³; or
two instances of R^{A}, two instances of R^{B}, two instances of R^{C}, an instance of R^{A} and an instance of R^{L}, or an instance of R^{C} and an instance of R^{L} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₅ saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R¹ is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R;
each instance of R² is independently C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each of m, n, p, q^{A}, q^{B}, q^{C}, r, and t is independently 0, 1, 2, 3, or 4.

In some embodiments, the present disclosure provides a compound of formula X-a, X-b, X-c, XI-a, XI-b, XI-c, XII-a, XII-b, XII-c, XIII-a, XIII-b, XIII-c, XIV-a, XIV-b, XIV-c, XV-a, XV-b, XV-c, XVI-a, XVI-b, XVI-c, XVII-a, XVII-b, XVII-c, XVIII-a, XVIII-b, XVIII-c, XIX-a, XIX-b, XIX-c, XX-a, XX-b, XX-c, XXI-a, XXI-b, XXI-c, XXII-a, XXII-b, XXII-c, XXIII-a, XXIII-b, XXIII-c, XXIV-a, XXIV-b, XXIV-c, XXV-a, XXV-b, or XXV-c:

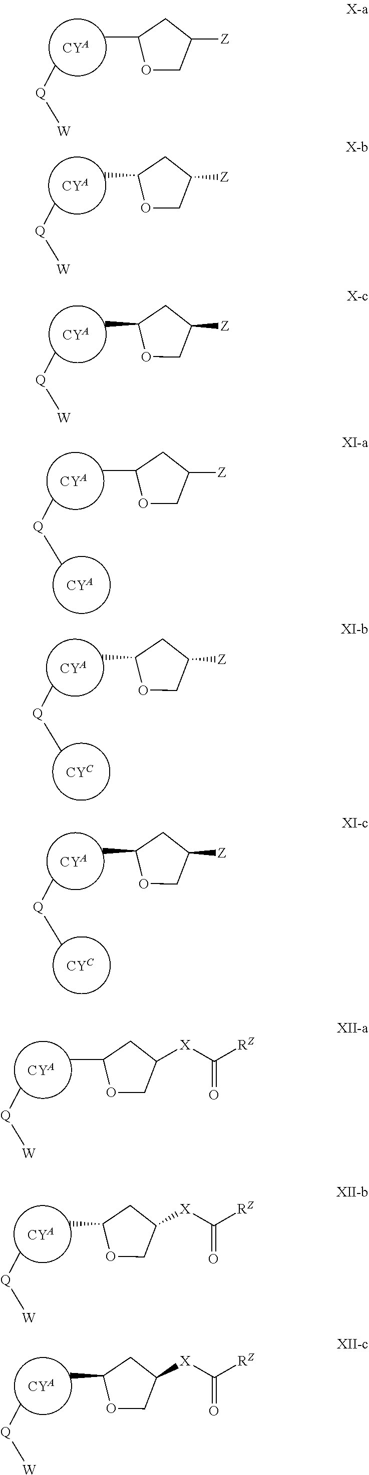

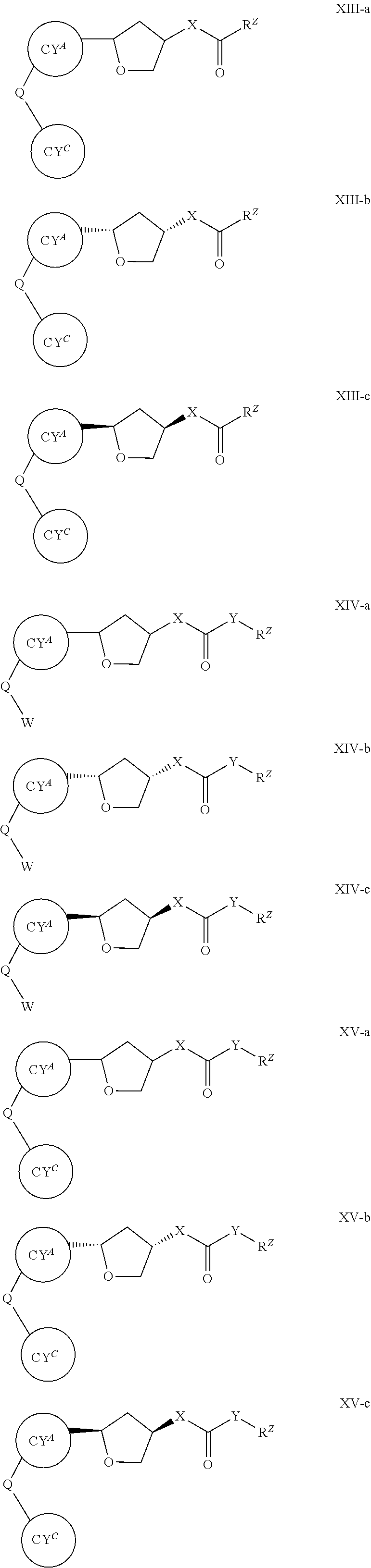

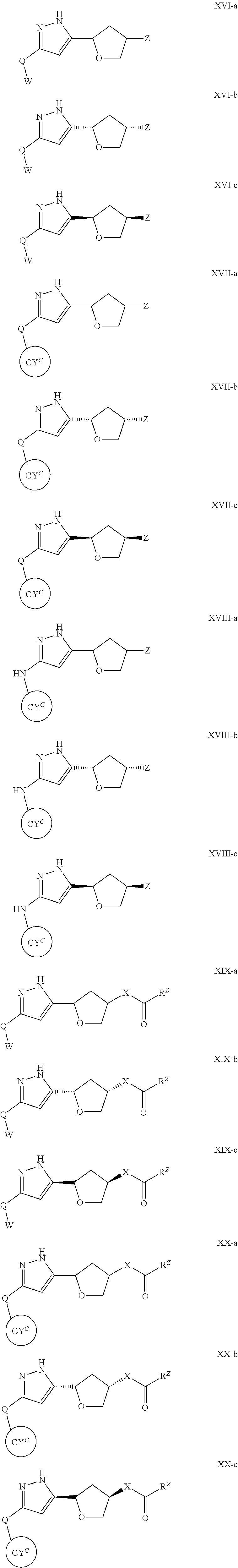

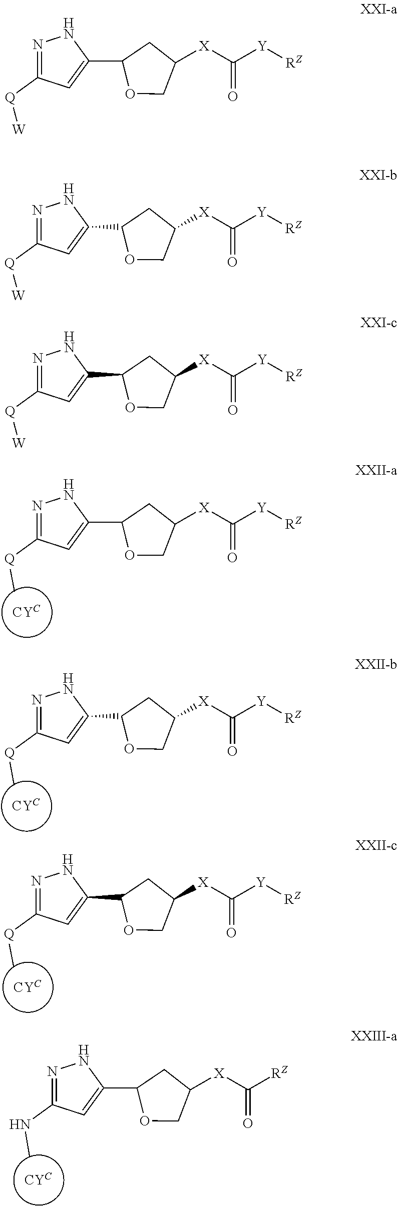

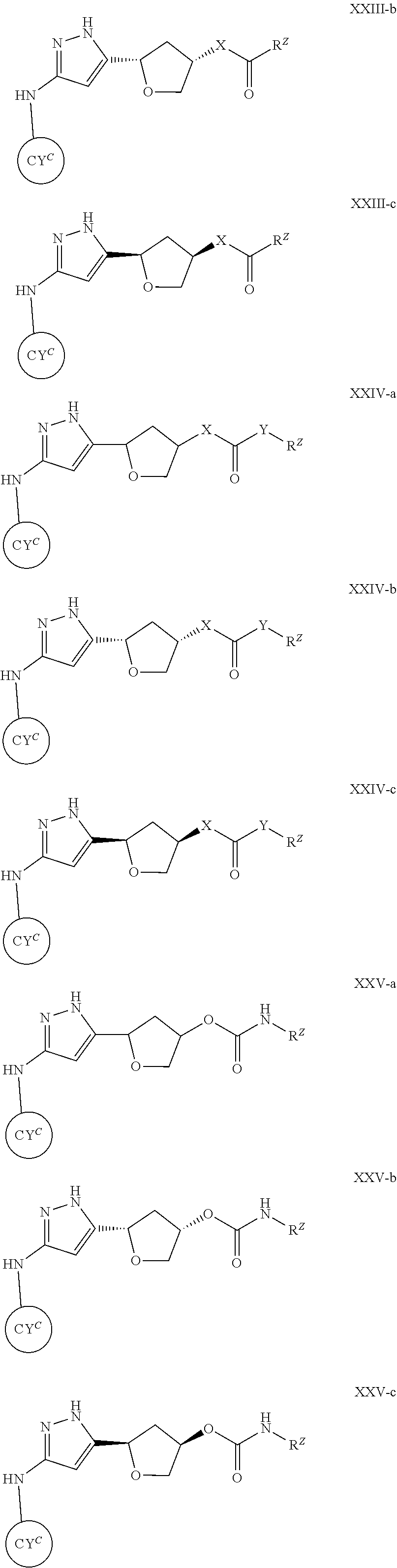

or a pharmaceutically acceptable salt thereof, wherein each of Cy^{A}, Cy^{C}, Q, Z, W, X, Y, and R^{Z} is as defined in embodiments and classes and subclasses herein.

In some embodiments of formula VIII, IX, X-a, X-b, X-c, XII-a, XII-b, XII-c, XIV-a, XIV-b, XIV-c, XVI-a, XVI-b, XVI-c, XIX-a, XIX-b, XIX-c, XXI-a, XXI-b, or XXI-c, W is Cy^{C}.

In some embodiments of formula X-a, X-b, X-c, XI-a, XI-b, XI-c, XVI-a, XVI-b, XVI-c, XVII-a, XVII-b, XVII-c, XVIII-a, XVIII-b, or XVIII-c, wherein Z is L²-R^{Z}, wherein:
R^{Z} is hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur; and
L² is as defined in embodiments and classes and subclasses herein

In some embodiments of formula XII-a, XII-b, XII-c, XIII-a, XIII-b, XIII-c, XIV-a, XIV-b, XIV-c, XV-a, XV-b, XV-c, XIX-a, XIX-b, XIX-c, XX-a, XX-b, XX-c, XXI-a, XXI-b, XXI-c, XXII-a, XXII-b, XXII-c, XXIII-a, XXIII-b, XXIII-c, XXIV-a, XXIV-b, XXIV-c, XXV-a, XXV-b, or XXV-c, R^{Z} is hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, the present disclosure provides a compound of formula XXVI-a, XXVI-b, XXVI-c, XXVII-a, XXVII-b, XXVII-c, XXVIII-a, XXVIII-b, or XXVIII-c:

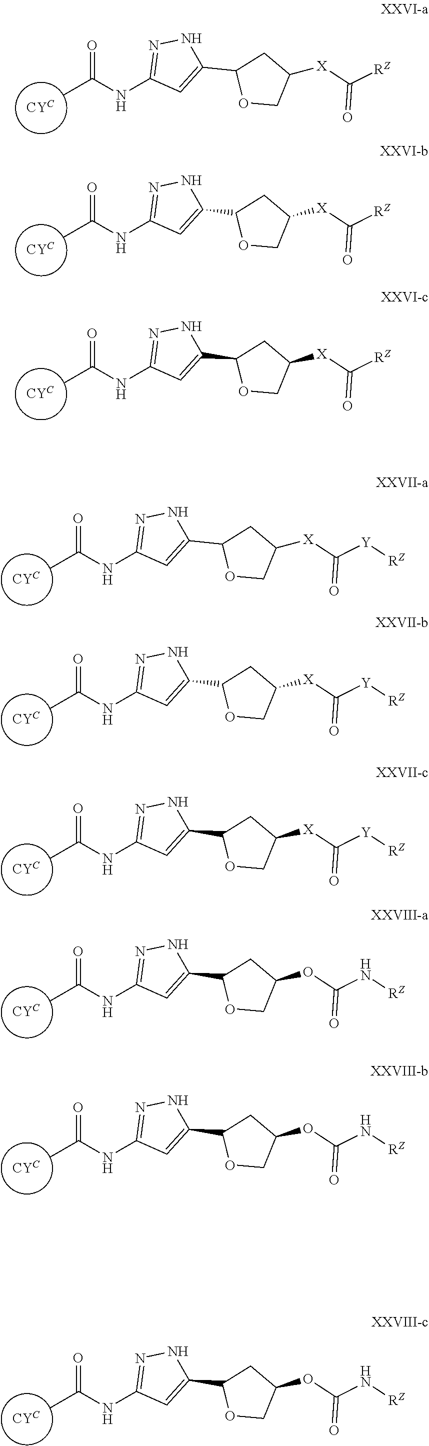

or a pharmaceutically acceptable salt thereof, wherein each of Cy^{C}, X, Y, and R^{Z} is as defined in embodiments and classes and subclasses herein.

As defined generally above, Cy^{A} is a 5-6 membered heteroarylene having 1-4 heteroatoms independently selected from nitrogen, oxygen, and sulfur; wherein Cy^{A} is substituted with m instances of R^{A} in addition to Q and Cy^{B}. In some embodiments, Cy^{A} is

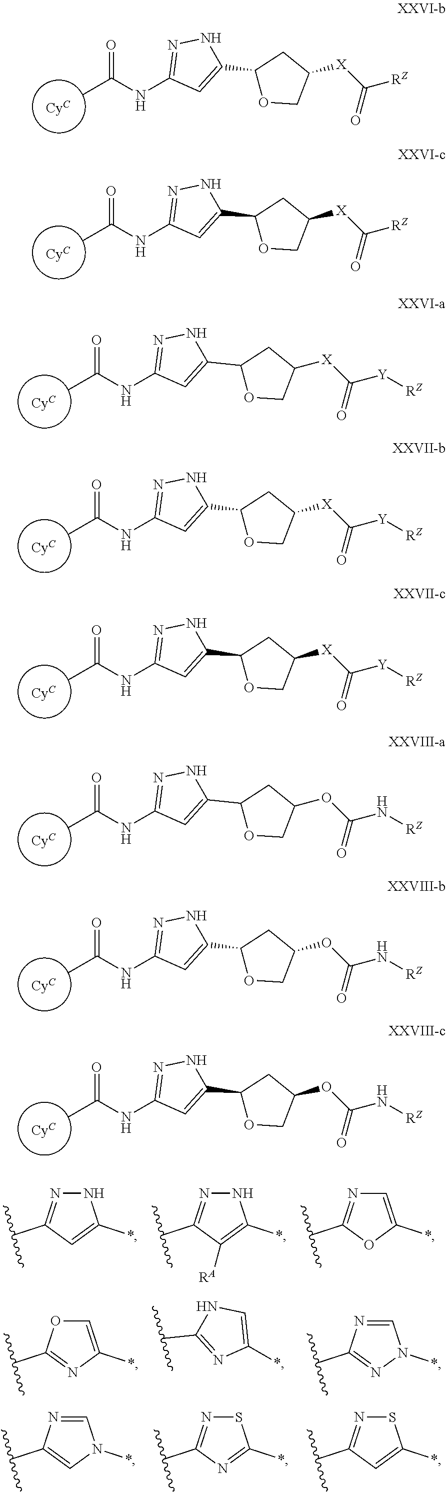

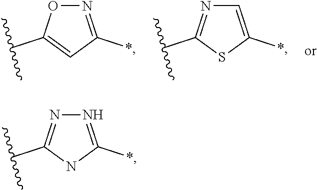

wherein

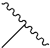

represents a covalent bond to Q and [symbol] represents a covalent bond to Cy^{B}. In some embodiments, Cy^{A} is

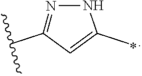 [SMILES: *c1cc(C)[nH]n1]

In some embodiments, Cy^{A} is

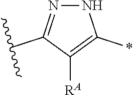

In some embodiments, Cy^{A} is

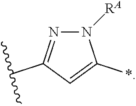

In some embodiments, Cy^{A} is

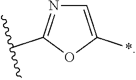

In some embodiments, Cy^{A} is

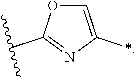

In some embodiments, Cy^{A} is

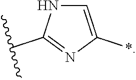

In some embodiments, Cy^{A} is

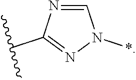

In some embodiments, Cy^{A} is

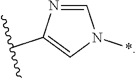

In some embodiments, Cy^{A} is

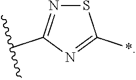

In some embodiments Cy^{A} is

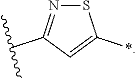

In some embodiments, Cy^{A} is

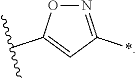

In some embodiments, Cy^{A} is

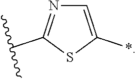

In some embodiments, Cy^{A} is

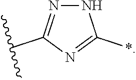

In some embodiments, Cy^{A} is selected from the groups depicted in the compounds in Table 1.

As defined generally above, Cy^{B} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenylene; or a 5-14 membered heteroarylene having 1-4 heteroatoms independently selected from nitrogen, oxygen, and sulfur; wherein Cy^{B} is substituted with n instances of R^{B} in addition to Cy^{A} and P. In some embodiments of any of formulae II, III, IV, V, VI, and VII, Cy^{B} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenylene; or a 5-14 membered heteroarylene having 1-4 heteroatoms independently selected from nitrogen, oxygen, and sulfur; wherein Cy^{B} is substituted with n instances of R^{B} in addition to Cy^{A} and P or X. In some embodiments of formulae I-A, Cy^{B} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenylene; or a 5-14 membered heteroarylene having 1-4 heteroatoms independently selected from nitrogen, oxygen, and sulfur; wherein Cy^{B} is substituted with n instances of R^{B} in addition to Cy^{A} and Z.

In some embodiments, Cy^{B} is a 3-7 membered saturated or partially unsaturated monocyclic carbocyclic ring. In some embodiments, Cy^{B} is a saturated or partially unsaturated 3-14 membered heterocyclic ring having 1-4 heteroatoms independently selected from nitrogen, oxygen, and sulfur. In some embodiments, Cy^{B} is phenylene. In some embodiments, Cy^{B} is a 5-14 membered heteroarylene having 1-4 heteroatoms independently selected from nitrogen, oxygen, and sulfur.

In some embodiments, Cy^{B} is a 3-7 membered saturated carbocyclic ring. In some embodiments, Cy^{B} is a cyclopropylene ring. In some embodiments, Cy^{B} is a cyclobutylene ring.

In some embodiments, Cy^{B} is a cyclopentylene ring. In some embodiments, Cy^{B} is a cyclohexylene ring. In some embodiments, Cy^{B} is a cycloheptylene ring.

In some embodiments, Cy^{B} is

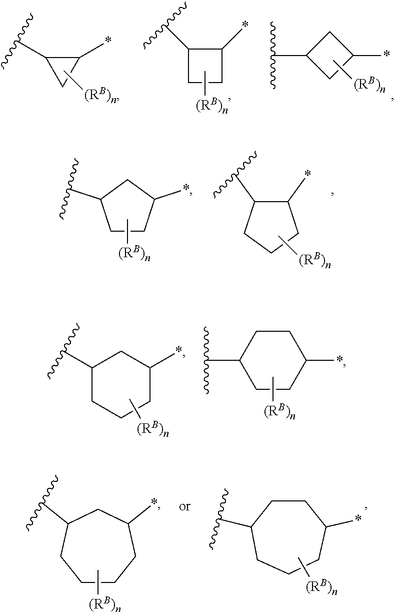

wherein [symbol]
represents a covalent bond to Cy^{A} and [symbol] represents a covalent bond to P, X, or Z. In some embodiments, Cy^{B} is

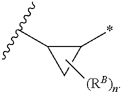

In some embodiments, Cy^{B} is

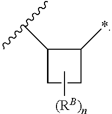

In some embodiments, Cy^{B} is

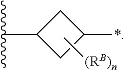

In some embodiments, Cy^{B} is

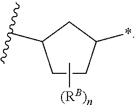

In some embodiments, Cy^{B} is

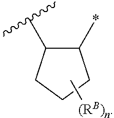

In some embodiments Cy^{B} is

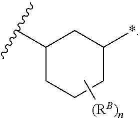

In some embodiments, Cy^{B} is

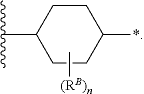

In some embodiments, Cy^{B} is

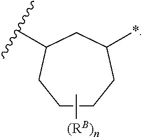

In some embodiments, Cy^{B} is

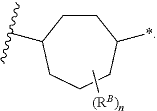

In some embodiments of Cy^{B}, Cy^{A} and P, X, or Z are in a trans-relationship. In some embodiments of Cy^{B}, Cy^{A} and P, X, or Z are in a cis-relationship. See, for example, the Cy^{B} groups depicted in the compounds in Table 1.

In some embodiments, Cy^{B} is a 5-8 membered saturated or partially unsaturated bridged bicyclic or fused carbocyclic ring. In some embodiments, Cy^{B} is a 5-8 membered saturated bridged bicyclic or fused carbocyclic ring. In some embodiments, Cy^{B} is a 6-7 membered saturated bridged bicyclic or fused carbocyclic ring. In some embodiments, Cy^{B} is

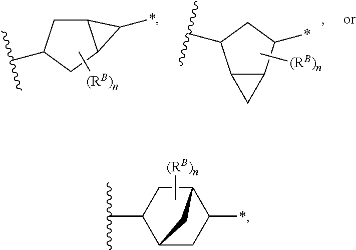

wherein [symbol] represents a covalent bond to Cy^{A} and [symbol] represents a covalent bond to P, X, or Z. In some embodiments, Cy^{B} is

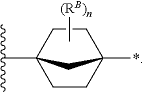

In some embodiments, Cy^{B} is

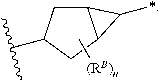

In some embodiments, Cy^{B} is a 3-7 membered partially unsaturated carbocyclic ring. In some embodiments, Cy^{B} is a 5-6 membered partially unsaturated carbocyclic ring. In some embodiments, Cy^{B} is

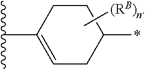

In some embodiments, Cy^{B} is

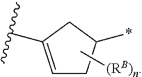

In some embodiments, Cy^{B} is a saturated or partially unsaturated 3-7 membered heterocyclic ring having 1-4 heteroatoms independently selected from nitrogen, oxygen, and sulfur. In some embodiments, Cy^{B} is a saturated 3-7 membered monocyclic heterocyclic ring having 1-4 heteroatoms independently selected from nitrogen, oxygen, and sulfur. In some embodiments, Cy^{B} is a saturated 4-7 membered monocyclic heterocyclic ring having 1-2 heteroatoms independently selected from nitrogen, oxygen, and sulfur.

In some embodiments, Cy^{B} is

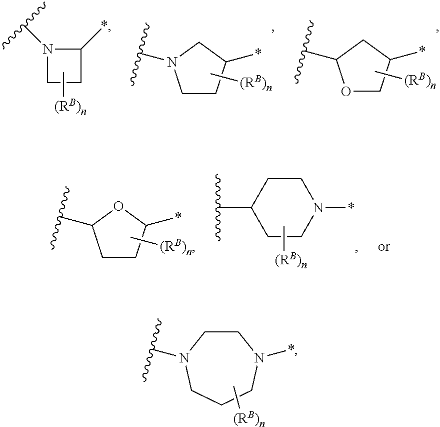

wherein [symbol] represents a covalent bond to Cy^{A} and [symbol] represents a covalent bond to P, X, or Z.

In some embodiments, Cy^{B} is

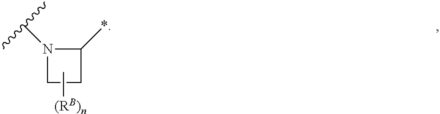

In some embodiments, Cy^{B} is

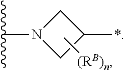

In some embodiments, Cy^{B} is

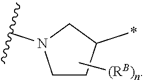

In some embodiments, Cy^{B} is

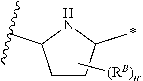

In some embodiments, Cy^{B} is

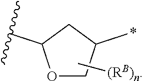

In some embodiments, Cy^{B} is

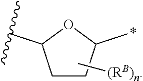

In some embodiments, Cy^{B} is

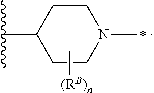

In some embodiments, Cy^{B} is

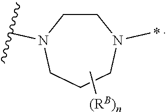

In some embodiments, Cy^{B} is a 5-14 membered heteroarylene having 1-4 heteroatoms independently selected from nitrogen, oxygen, and sulfur. In some embodiments, Cy^{B} is a 9-membered heteroarylene having 1-4 heteroatoms independently selected from nitrogen, oxygen, and sulfur. In some embodiments, Cy^{B} is selected from

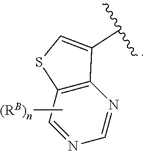

In some embodiments, Cy^{B} is a saturated 6-10 membered bridged bicyclic or fused heterocyclic ring having 1-4 heteroatoms independently selected from nitrogen, oxygen, and sulfur. In some embodiments, Cy^{B} is a saturated 6-10 membered bridged bicyclic or fused heterocyclic ring having 1-2 heteroatoms independently selected from nitrogen, oxygen, and sulfur. In some embodiments, Cy^{B} is a saturated 6-10 membered bridged bicyclic heterocyclic ring having 1-2 heteroatoms independently selected from nitrogen, oxygen, and sulfur. In some embodiments, Cy^{B} is a saturated 7-8 membered bridged bicyclic heterocyclic ring having 1-2 heteroatoms independently selected from nitrogen, oxygen, and sulfur.

In some embodiments, Cy^{B} is a saturated or partially unsaturated 6-10 membered spirofused heterocyclic ring having 1-2 heteroatoms independently selected from nitrogen, oxygen, and sulfur. In some embodiments, Cy^{B} is a saturated or partially unsaturated 6-9 membered spirofused heterocyclic ring having 1-2 heteroatoms independently selected from nitrogen, oxygen, and sulfur.

In some embodiments, Cy^{B} is

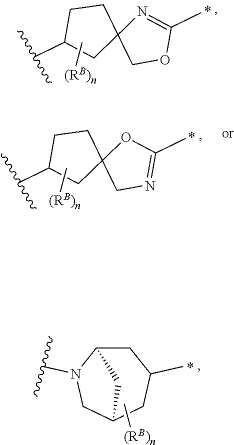

wherein

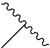

represents a covalent bond to Cy^{A} and [symbol] represents a covalent bond to P, X, or Z. In some embodiments, Cy^{B} is

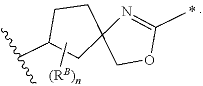

In some embodiments, Cy^{B} is

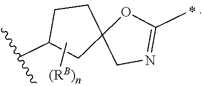

In some embodiments, Cy^{B} is

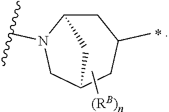

n some embodiments, Cy^{B} is

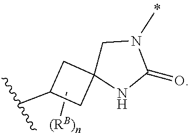

In some embodiments, Cy^{B} is

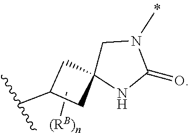

In some embodiments, Cy^{B} is

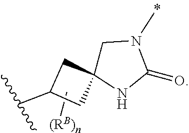

In some embodiments, Cy^{B} is selected from the groups depicted in the compounds in Table 1.

As defined generally above, P is hydrogen or —L²-R^{P}, wherein each of L² and R^{P} is as defined in embodiments and classes and subclasses herein. In some embodiments, P is hydrogen. In some embodiments, P is —L²-R^{P}. In some embodiments, P is —OR^{P}, —NHR, —SR^{P}, —NHC(O)NHR, —OC(O)NHR, and —NHC(O)OR^{P}.

In some embodiments, P is —XC(O)Y^{P}, wherein each of X, Y, and R is as defined in embodiments and classes and subclasses herein. In some embodiments, each P is selected from the groups depicted in the compounds in Table 1.

As defined generally above, Z is hydrogen or —L²-R^{Z}, wherein each of L² and R^{Z} is as defined in embodiments and classes and subclasses herein. In some embodiments, Z is hydrogen. In some embodiments, Z is —L²-R^{Z}. In some embodiments, Z is —OR^{Z}, —NHR^{Z}, —SR^{Z}, —NHC(O)NHR^{Z}, —OC(O)NHR^{Z}, and —NHC(O)OR^{Z}. In some embodiments, each Z is selected from the groups depicted in the compounds in Table 1.

As defined generally above, R is R, wherein R is as defined in embodiments and classes and subclasses herein. In some embodiments, R^{P} is hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur. In some embodiments, R^{P} is hydrogen. In some embodiments, R^{P} is an optionally substituted C₁₋₆ aliphatic. In some such embodiments, R^{P} is optionally substituted C₁₋₄ aliphatic. In some embodiments, R^{P} is —CH₃, —CH₂CH₃, —CH₂CH₂CH₃, —CH(CH₃)₂, or —CH₂CH(CH₃)₂, —C(CH₃)₃,

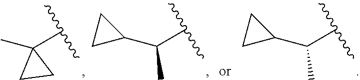

In some embodiments, R^{P} is C₁₋₆ aliphatic optionally substituted with —R^{∘} or —OR^{∘}. In some embodiments, R^{P} is

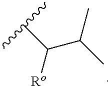

In some embodiments, R^{P}
is

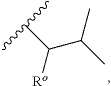

wherein R^{∘} is —OR^{∘} or —N(R^{∘})₂. In some embodiments, R^{P} is

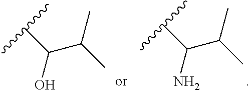

In some embodiments, R^{P} is

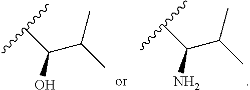

In some embodiments, R^{P} is

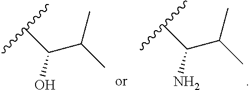

In some embodiments, R^{P} is an optionally substituted saturated or partially unsaturated 3-7 membered carbocyclic ring. In some embodiments, R^{P} is an optionally substituted saturated 3-membered carbocyclic ring. In some such embodiments, R^{P} is

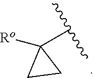

In some such embodiments, R^{∘} is optionally substituted with halogen.

In some embodiments, R^{P} is an optionally substituted phenyl ring. In some embodiments, R is an optionally substituted 3-7 membered saturated or partially unsaturated heterocyclic ring having 1-2 heteroatoms independently selected from nitrogen, oxygen, and sulfur. In some embodiments, R is an optionally substituted 4-membered saturated heterocyclic ring having 1 heteroatom selected from nitrogen, oxygen, and sulfur. In some such embodiments, R^{P} is

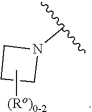

In some such embodiments, R^{P} is

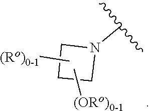

In some embodiments, R^{P} is an optionally substituted 5-6 membered saturated or partially unsaturated heterocyclic ring having 1-2 heteroatoms independently selected from nitrogen, oxygen, and sulfur. In some embodiments, R^{P} is an optionally substituted 5-membered saturated or partially unsaturated heterocyclic ring having 1-2 heteroatoms independently selected from nitrogen, oxygen, and sulfur. In some such embodiments, R^{P} is

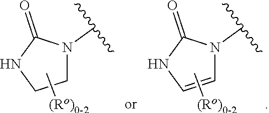

In some such embodiments, R^{P} is

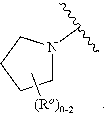

In some embodiments, R^{P} is an optionally substituted 6-membered saturated or partially unsaturated heterocyclic ring having 1-2 heteroatoms independently selected from nitrogen, oxygen, and sulfur. In some such embodiments, R^{P} is

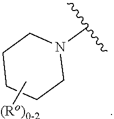

In some embodiments, R^{P} is an optionally substituted 5-6 membered heteroaryl ring having 1-4 heteroatoms independently selected from nitrogen, oxygen, and sulfur. In some embodiments, R is an optionally substituted 5-membered heteroaryl ring having 1-4 heteroatoms independently selected from nitrogen, oxygen, and sulfur. In some embodiments, R is an optionally substituted 6-membered heteroaryl ring having 1-2 nitrogen atoms. In some embodiments, R^{P} is selected from optionally substituted isothiazolyl, pyridinyl, or pyridazinyl. In some such embodiments, R^{P} is

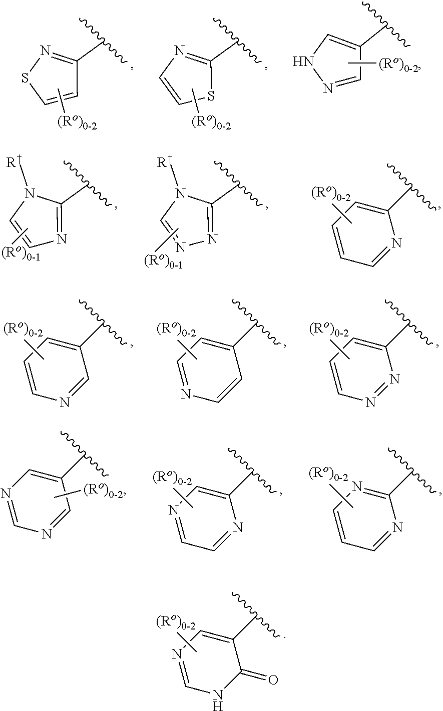

In some embodiments, R is selected from the groups depicted in the compounds in Table 1.

As defined generally above, R^{Z} is hydrogen, or an optionally substituted group selected from C₁₋₈ aliphatic, a saturated or partially unsaturated 3-14 membered carbocyclic ring, phenyl, a 3-10 membered saturated or partially unsaturated heterocyclic ring having 1-4 heteroatoms independently selected from nitrogen, oxygen, and sulfur, and a 5-14 membered heteroaryl ring having 1-4 heteroatoms independently selected from nitrogen, oxygen, and sulfur.

In some embodiments, R^{Z} is hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur. In some embodiments, R^{Z} is hydrogen. In some embodiments, R^{Z} is an optionally substituted C₁₋₆ aliphatic. In some such embodiments, R^{Z} is optionally substituted C₁₋₄ aliphatic. In some embodiments, R^{Z} is —CH₃, —CH₂CH₃, —CH₂CH₂CH₃, —CH(CH₃)₂, or —CH₂CH(CH₃)₂, —C(CH₃)₃,

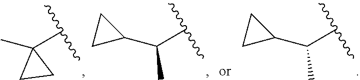

In some embodiments, R^{Z} is C₁₋₆ aliphatic optionally substituted with —R^{∘} or —OR^{∘}. In some embodiments, R^{Z} is

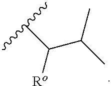

In some embodiments, R^{Z} is

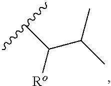

wherein R^{∘} is —OR^{∘} or —N(R^{∘})₂. In some embodiments, R^{Z} is

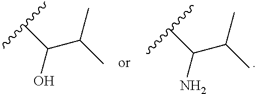

In some embodiments, R^{Z} is

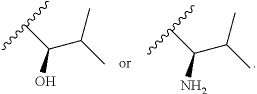

In some embodiments, R^{Z} is

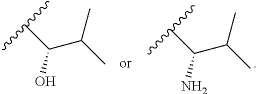

In some embodiments, R^{Z} is an optionally substituted saturated or partially unsaturated 3-7 membered carbocyclic ring. In some embodiments, R^{Z} is an optionally substituted saturated 3-membered carbocyclic ring. In some such embodiments, R^{Z} is

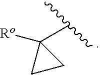

In some such embodiments, R^{∘} is optionally substituted with halogen.

In some embodiments, R^{Z} is an optionally substituted phenyl ring. In some embodiments, R^{Z} is an optionally substituted 3-7 membered saturated or partially unsaturated heterocyclic ring having 1-2 heteroatoms independently selected from nitrogen, oxygen, and sulfur. In some embodiments, R^{Z} is an optionally substituted 4-membered saturated heterocyclic ring having 1 heteroatom selected from nitrogen, oxygen, and sulfur. In some such embodiments, R^{Z} is

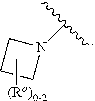

In some such embodiments, R^{Z} is

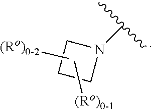

In some embodiments, R^{Z} is an optionally substituted 5-6 membered saturated or partially unsaturated heterocyclic ring having 1-2 heteroatoms independently selected from nitrogen, oxygen, and sulfur. In some embodiments, R^{Z} is an optionally substituted 5-membered saturated or partially unsaturated heterocyclic ring having 1-2 heteroatoms independently selected from nitrogen, oxygen, and sulfur. In some such embodiments, R^{Z} is

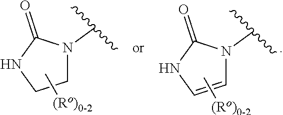

In some such embodiments, R^{Z} is

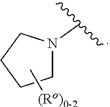

In some embodiments, R^{Z} is an optionally substituted 6-membered saturated or partially unsaturated heterocyclic ring having 1-2 heteroatoms independently selected from nitrogen, oxygen, and sulfur. In some such embodiments, R^{Z} is

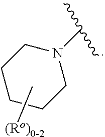

In some embodiments, R^{Z} is an optionally substituted 5-6 membered heteroaryl ring having 1-4 heteroatoms independently selected from nitrogen, oxygen, and sulfur. In some embodiments, R^{Z} is an optionally substituted 5-membered heteroaryl ring having 1-4 heteroatoms independently selected from nitrogen, oxygen, and sulfur. In some embodiments, R^{Z} is an optionally substituted 6-membered heteroaryl ring having 1-2 nitrogen atoms. In some embodiments, R^{Z} is selected from optionally substituted isothiazolyl, pyridinyl, or pyridazinyl. In some such embodiments, R^{Z} is

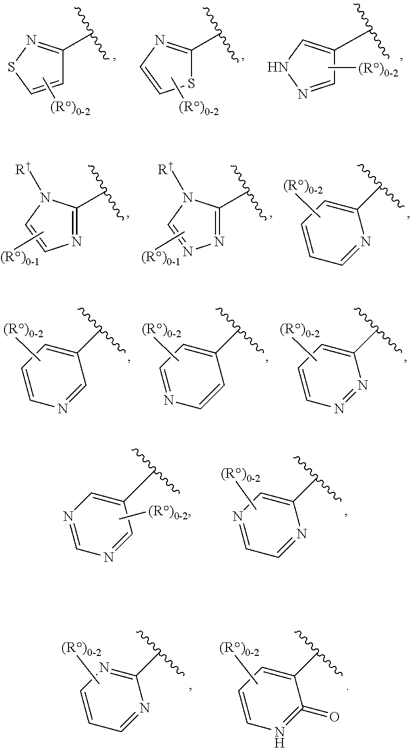

In some embodiments, R^{Z} is an optionally substituted group selected from C₁₋₈ aliphatic, a saturated or partially unsaturated 3-14 membered carbocyclic ring, phenyl, a 3-10 membered saturated or partially unsaturated heterocyclic ring having 1-4 heteroatoms independently selected from nitrogen, oxygen, and sulfur, and a 5-14 membered heteroaryl ring having 1-4 heteroatoms independently selected from nitrogen, oxygen, and sulfur.

In some embodiments, R^{Z} is optionally substituted C₁₋₈ aliphatic. In some embodiments, R^{Z} is an optionally substituted saturated or partially unsaturated 3-14 membered carbocyclic ring. In some embodiments, R^{Z} is an optionally substituted 3-10 membered saturated or partially unsaturated heterocyclic ring having 1-4 heteroatoms independently selected from nitrogen, oxygen, and sulfur. In some embodiments, R^{Z} is an optionally substituted 5-14 membered heteroaryl ring having 1-4 heteroatoms independently selected from nitrogen, oxygen, and sulfur. In some embodiments, R^{Z} is an optionally substituted 10-membered heteroaryl ring having 1-4 heteroatoms independently selected from nitrogen, oxygen, and sulfur. In some embodiments, R^{Z} is an optionally substituted 10-membered heteroaryl ring having 1-4 nitrogen heteroatoms. In some embodiments, R^{Z} is an optionally substituted 10-membered heteroaryl ring having 3 nitrogen heteroatoms. In some embodiments, R^{Z} is pyrido[3,4-d]pyridazine.

In some embodiments, R^{Z} is selected from the groups depicted in the compounds in Table 1.

As defined generally above, L² is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic.

In some embodiments, L² is a covalent bond. In some embodiments, L² is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic. In some embodiments, L² is —CH₂—. In some embodiments, L² is —CH₂ O—.

In some embodiments, L² is —O—, —NH—, —S—, —NHC(O)NH—, —N(CH₃)C(O)NH—, —OC(O)NH—, —OC(O)N(CH₃)—, —NHC(O)O—, —CH₂C(O)NH—, —CH₂ OC(O)NH—, —C(O)NH—, and —C(O)O—. In some embodiments, L² is —O—, —NH—, —S—, —NHC(O)NH—, —N(CH₃)C(O)NH—, —OC(O)NH—, —OC(O)N(CH₃)—, —NHC(O)O—, —CH₂C(O)NH—, —NHC(O)CH₂—, —CH₂ OC(O)NH—, —C(O)NH—, —NHC(O)—, —C(O)O—, —OC(O)—, —NHS(O)₂—, —NHS(O)₂NH—, and —OC(O)N(Pr)—. In some embodiments, L² is —O—. In some embodiments, L² is —NH—. In some embodiments, L² is —S—. In some embodiments, L² is —NHC(O)NH—. In some embodiments, L² is —N(CH₃)C(O)NH—. In some embodiments, L² is —OC(O)NH—. In some embodiments, L² is —NHC(O)O—. In some embodiments, L² is-CH₂C(O)NH—. In some embodiments, L² is —CH₂ OC(O)NH—. In some embodiments, L² is —C(O)NH—. In some embodiments, L² is —NHC(O)—. In some embodiments, L² is —C(O)O—. In some embodiments, L² is —OC(O)—. In some embodiments, L² is —NHC(O)CH₂—. In some embodiments, L² is —NHS(O)₂—. In some embodiments, L² is —NHS(O)₂NH—. In some embodiments, L² is —OC(O)N(Pr)—. In some embodiments, L² is a covalent bond, —CH₂—, —NH—, —O—, —NHC(O)NH—,

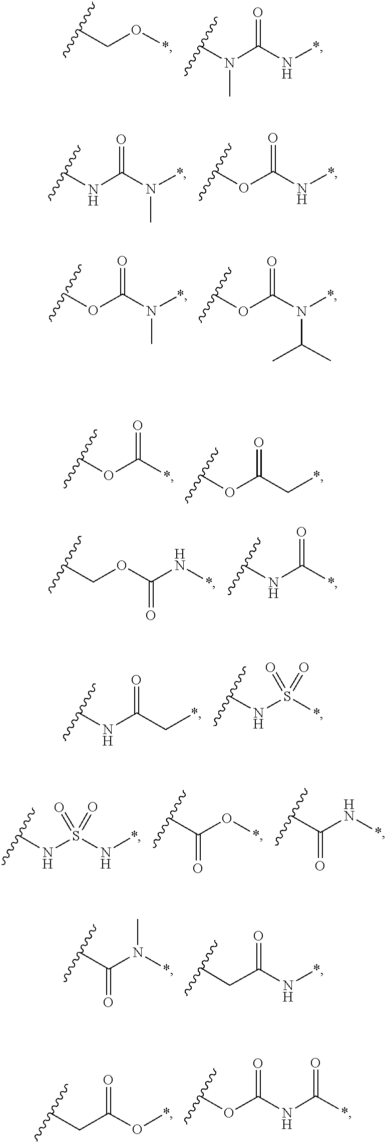

wherein

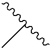

represents a covalent bond to Cy^{B} and [symbol] represents a covalent bond to R^{P} or R^{Z}. In some embodiments, L² is

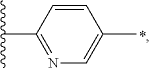 [SMILES: *c1ccc(C)cn1]

wherein

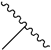

represents a covalent bond to Cy^{B} and [symbol] represents a covalent bond to R^{P} or R^{Z}. In some embodiments, L² is

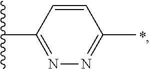 [SMILES: *c1ccc(C)nn1]

wherein

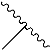

represents a covalent bond to Cy^{B} and [symbol] represents a covalent bond to R^{P} or R^{Z}.

In some embodiments, L² is —XC(O)Y—, wherein each of X and Y is as defined in embodiments and classes and subclasses herein. In some embodiments, X is —O—. In some embodiments, X is —NR^{X}—. In some embodiments, X is —NH—. In some embodiments, X is —N(CH₃)—. In some embodiments, X is —S—. In some embodiments, Y is —O—. In some embodiments, Y is —NR^{Y}—. In some embodiments, Y is —NH—. In some embodiments, Y is —N(CH₃)—. In some embodiments, Y is —S—.

In some embodiments, each L² is selected from the groups depicted in the compounds in Table 1.

As defined generally above, Q is L¹, wherein L¹ is as defined in embodiments and classes and subclasses herein. In some embodiments, Q is —NH—,

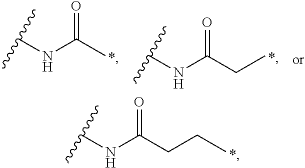

wherein

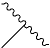

represents a covalent bond to Cy^{A} and [symbol] represents a covalent bond to Cy^{C} or W. In some embodiments, Q is —NH—. In some embodiments, Q is —O—. In some embodiments, Q is

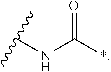

In some embodiments, Q is

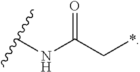

In some embodiments, Q is

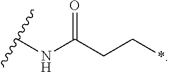

In some embodiments, Q is —NHC(O)NH—. In some embodiments, Q is

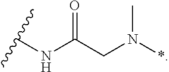

In some embodiments, Q is selected from the groups depicted in the compounds in Table 1.

As defined generally above, L¹ is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic.

In some embodiments, L¹ is a covalent bond. In some embodiments, L¹ is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic.

In some embodiments, L¹ is a —NH—,

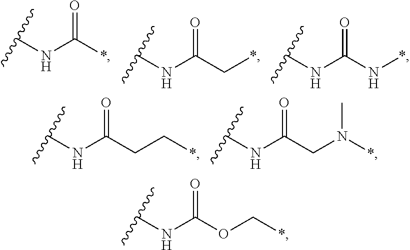

wherein

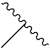

represents a covalent bond to Cy^{A} and [symbol] represents a covalent bond to Cy^{C} or W. In some embodiments, L¹ is —NH—. In some embodiments, L¹ is —O—. In some embodiments, L¹ is

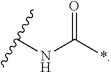

In some embodiments, L¹ is

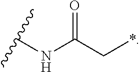

In some embodiments, L¹ is

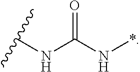

In some embodiments, L¹ is

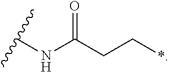

In some embodiments, L¹ is

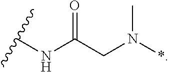

In some embodiments, L¹ is

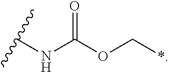

In some embodiments, L¹ is selected from the groups depicted in the compounds in Table 1.

As defined generally above, each instance of R^{L} is independently R¹ or R², and is substituted by t instances of R³. In some embodiments, R^{L} is R¹. In some embodiments, R^{L} is R².

As defined generally above, each instance of R^{A}, R^{B}, and R^{C} is independently R¹ or R² wherein R^{A} is substituted by q^{A} instances of R³, R^{B} is substituted by q^{B} instances of R³, and R^{C} is substituted by q^{C} instances of R³. In some embodiments, R^{A} is R¹. In some embodiments, R^{B} is R¹. In some embodiments, R^{C} is R¹. In some embodiments, R^{A} is R². In some embodiments, R^{B} is R². In some embodiments, R^{C} is R².

As defined generally above, each instance of R¹ (e.g., the R¹ group of R^{A}, the R¹ group of R^{B}, or the R¹ group of R^{C}) is independently oxo, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R. In some embodiments, R¹ is oxo. In some embodiments, each R¹ is independently halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R.

In some embodiments, R¹ is halogen, —CN, or —NO₂. In some embodiments, R¹ is —OR, —SR, or —NR₂. In some embodiments, R¹ is —S(O)₂R, —S(O)₂NR₂, —S(O)R, —S(O)NR₂, —C(O)R, —C(O)OR, —C(O)NR₂, —C(NR)NR₂, or —C(O)N(R)OR. In some embodiments, R¹ is —S(O)₂R, —S(O)₂N(H)R, —S(O)R, —S(O)N(H)R, —C(O)R, —C(O)OR, —C(O)N(H)R, —C(NH)N(H)R, or —C(O)N(H)OR. In some embodiments, R¹ is —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R. In some embodiments, R¹ is —OC(O)R, —OC(O)N(H)R, —N(H)C(O)OR, —N(H)C(O)R, —N(H)C(NH)R, —N(H)C(O)NR₂, —N(H)C(NH)NR₂, —N(H)S(O)₂NR₂, —N(H)S(O)R, or —N(H)S(O)₂R. In some embodiments, R^{A} is halogen. In some embodiments, R^{B} is halogen. In some embodiments, R^{B} is —C≡N. In some embodiments, R^{C} is —S(O)₂R. In some embodiments, R^{C} is —S(O)₂CH₃. In some embodiments, R^{C} is —OR. In some embodiments, R^{C} is —OCH₃. In some embodiments, R^{C} is oxo. In some embodiments, R^{C} is —N(R)C(O)R. In some such embodiments, R^{C} is —N(H)C(O)R. In some embodiments, R^{C} is —C≡N.

As defined generally above, each instance of R² (e.g., the R² group of R^{A}, the R² group of R^{B}, or the R² group of R^{C}) is independently C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur. In some embodiments, R² is C₁₋₇ aliphatic. In some embodiments, R² is phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur. In some embodiments, R² is phenyl.

In some embodiments, R² is a 5-6 membered monocyclic heteroaryl ring having 1-4 heteroatoms independently selected from nitrogen, oxygen, and sulfur. In some embodiments, R² is an 8-10 membered bicyclic heteroaryl ring having 1-4 heteroatoms independently selected from nitrogen, oxygen, and sulfur. In some embodiments, R² is a 3-7 membered saturated or partially unsaturated carbocyclic ring. In some embodiments, R² is a 3-7 membered saturated or partially unsaturated monocyclic heterocyclic ring having 1-2 heteroatoms independently selected from nitrogen, oxygen, and sulfur. In some embodiments, R² is a 7-12 membered saturated or partially unsaturated bicyclic heterocyclic ring having 1-4 heteroatoms independently selected from nitrogen, oxygen, and sulfur.

In some embodiments, R^{A} is C₁₋₇ aliphatic. In some such embodiments, R^{A} is —CH₃. In some embodiments, R^{A} is —C(CH₃)₃.

In some embodiments, R^{B} is C₁₋₇ aliphatic. In some such embodiments, R^{B} is —CH₃. In some embodiments, R^{B} is selected from —CH₃, —CH₂CH₃, —CH(CH₃)₂,

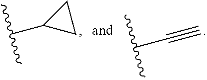

In some embodiments, R^{B} is C₁₋₇ aliphatic substituted with R³. In some embodiments, R^{B} is C₁₋₇ aliphatic substituted with R³, wherein R³ is —OR. In some embodiments, R^{B} is C₁₋₂ aliphatic substituted with R³, wherein R³ is —OR. In some embodiments, R^{B} is —CH₂OH. In some embodiments, R^{B} is oxo. In some embodiments, R^{B} is —OR, wherein R is C₁₋₆ aliphatic. In some embodiments, R^{B} is —OCH₃.

In some embodiments, R^{C} is C₁₋₇ aliphatic. In some such embodiments, R^{C} is —CH₃ or —C(CH₃)₃. In some embodiments, R^{c}—CH₂C(CH₃)₃. In some embodiments, R^{C} is C₁₋₇ aliphatic substituted with R³. In some embodiments, R^{C} is C₁₋₇ aliphatic substituted with R³, wherein R³ is —OR. In some embodiments, R^{C} is C₁₋₂ aliphatic substituted with R³, wherein R³ is —OR. In some embodiments R^{C} is —CH₂OCH₃. In some embodiments R^{C} is

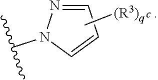

In some embodiments, R^{C} is —N(H)C(O)CH₃. In some embodiments, R^{C} is —C(O)OR. In some embodiments, R^{C} is —C(O)OR, wherein R is C₁₋₆ aliphatic. In some embodiments, R^{C} is —C(O)OCH₂CH₃. In some embodiments, R^{C} is C₁₋₇ aliphatic substituted with R³, wherein R³ is halogen. In some embodiments, R^{C} is C₁₋₇ aliphatic substituted with R³, wherein R³ is fluorine. In some embodiments, R^{C} is —CF₃. In some embodiments, R^{C} is oxo. In some embodiments, R^{C} is —OR substituted with R³. In some embodiments, R^{C} is —OR substituted with R³, wherein R is C₁₋₆ aliphatic and R³ is —OR. In some embodiments, R^{C} is —OCH₂CH₂OH. In some embodiments, R^{C} is C₁₋₇ aliphatic substituted with R³, wherein R³ is —OR. In some embodiments, R^{C} is C₁₋₇ aliphatic substituted with R³, wherein R³ is —OR. In some embodiments, R^{C} is C₁₋₇ aliphatic substituted with R³, wherein R³ is —OR and R is C₁₋₆ aliphatic, optionally substituted with halogen.

As defined generally above, each instance of R³ is independently oxo, halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur. In some embodiments, R³ is oxo. In some embodiments, R³ is halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur. In some embodiments, R³ is halogen, —CN, or —NO₂. In some embodiments, R³ is —OR, —SR, or —NR₂. In some embodiments, R³ is —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, or —C(O)N(R)OR. In some embodiments, R³ is —S(O)₂R, —S(O)₂N(H)R, —S(O)R, —S(O)N(H)R, —S(O)₂F, —OS(O)₂F, —C(O)R, —C(O)OR, —C(O)N(H)R, —C(NH)NR₂, or —C(O)N(H)OR. In some embodiments, R³ is OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R. In some embodiments, R³ is —OC(O)R, —OC(O)N(H)R, —N(H)C(O)OR, —N(H)C(O)R, —N(H)C(NH)R, —N(H)C(O)NR₂, —N(H)C(NH)NR₂, —N(H)S(O)₂NR₂, —N(H)S(O)R, or —N(H)S(O)₂R. In some embodiments, R³ is an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur. In some embodiments, R³ is optionally substituted C₁₋₆ aliphatic. In some embodiments, R³ is optionally substituted phenyl. In some embodiments, R³ is an optionally substituted 3-7 membered saturated or partially unsaturated heterocyclic ring having 1-2 heteroatoms independently selected from nitrogen, oxygen, and sulfur. In some embodiments, R³ is an optionally substituted 5-6 membered heteroaryl ring having 1-4 heteroatoms independently selected from nitrogen, oxygen, and sulfur.

As defined generally above, W is hydrogen or Cy^{C}. In some embodiments, W is hydrogen. In some embodiments, W is Cy^{C}.

As defined generally above, Cy^{C} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenyl; or a 5-14 membered heteroaryl having 1-4 heteroatoms independently selected from nitrogen, oxygen, and sulfur; wherein Cy^{C} is substituted with p instances of R^{C} in addition to Q. In some embodiments, Cy^{C} is a saturated or partially unsaturated 3-14 membered carbocyclic ring. In some embodiments, Cy^{C} is a saturated or partially unsaturated 3-7 membered monocyclic carbocyclic ring. In some embodiments, Cy^{C} is cyclopropyl.

In some embodiments, Cy^{C} is a saturated or partially unsaturated 3-14 membered heterocyclic ring having 1-4 heteroatoms independently selected from nitrogen, oxygen, and sulfur. In some embodiments, Cy^{C} is phenyl. In some embodiments, Cy^{C} is a 5-14 membered heteroaryl having 1-4 heteroatoms independently selected from nitrogen, oxygen, and sulfur.

In some embodiments, Cy^{C} is a 5-6 membered heteroaryl ring having 1-3 heteroatoms selected from nitrogen, oxygen, and sulfur. In some embodiments, Cy^{C} is a 5-membered heteroaryl ring having 1-3 heteroatoms selected from nitrogen, oxygen, and sulfur. In some embodiments, Cy^{C} is a 5-membered heteroaryl ring having 1-2 heteroatoms selected from nitrogen, oxygen, and sulfur. In some embodiments, Cy^{C} is

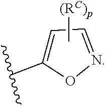

In some embodiments, Cy^{C} is

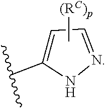

In some embodiments, Cy^{C} is

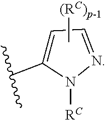

In some embodiments, Cy^{C} is

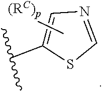

In some embodiments, Cy^{C} is

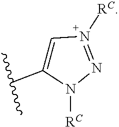

In some embodiments, Cy^{C} is

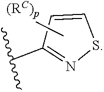

In some embodiments, Cy^{C} is a 6-membered heteroaryl ring having 1-3 nitrogen atoms. In some embodiments, Cy^{C} is pyridyl. In some embodiments, Cy^{C} is pyrimidinyl. In some embodiments, Cy^{C} is pyridazinyl. In some embodiments, Cy^{C} is

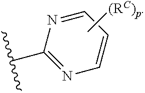

In some embodiments, Cy^{C} is

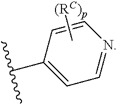

In some embodiments, Cy^{C} is

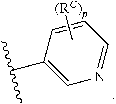

In some embodiments, Cy^{C} is

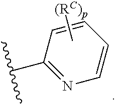

In some embodiments, Cy^{C} is

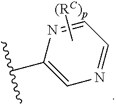

In some embodiments, Cy^{C} is

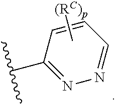

In some embodiments, Cy^{C} is a 9-10 membered heteroaryl having 1-4 heteroatoms independently selected from nitrogen, oxygen, and sulfur. In some embodiments, Cy^{C} is a 9-10 membered heteroaryl having 1-3 heteroatoms independently selected from nitrogen, oxygen, and sulfur. In some embodiments, Cy^{C} is a 9-10 membered heteroaryl having 2-4 nitrogen atoms. In some embodiments, Cy^{C} is

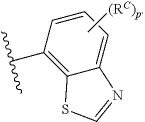

In some embodiments, Cy^{C} is

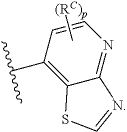

In some embodiments, Cy^{C} is

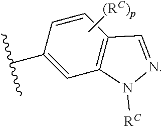

In some embodiments, Cy^{C} is

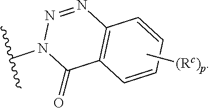

In some embodiments, Cy^{C} is

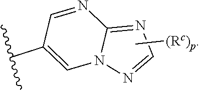

In some embodiments, Cy^{C} is

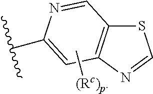

In some embodiments, Cy^{C} is

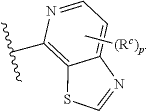

In some embodiments, Cy^{C} is

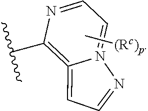

In some embodiments Cy^{C} is

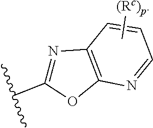

In some embodiments, Cy^{C} is

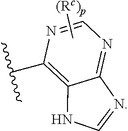

In some embodiments, Cy^{C} is

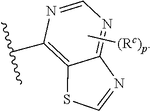

In some embodiments, Cy^{C} is

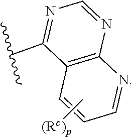

In some embodiments, Cy^{C} is

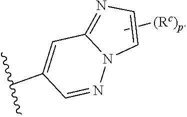

In some embodiments, Cy^{C} is

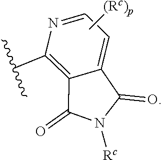

In some embodiments, Cy^{C} is

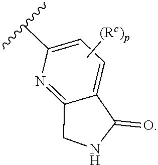

In some embodiments, Cy^{C} is

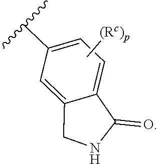

As defined generally above, each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur. In some embodiments, R is hydrogen. In some embodiments, R is an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur. In some embodiments, R is optionally substituted C₁₋₆ aliphatic. In some embodiments, R is an optionally substituted saturated or partially unsaturated 3-7 membered carbocyclic ring. In some embodiments, R is optionally substituted phenyl. In some embodiments, R is an optionally substituted 3-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur.

As defined generally above, m is 0, 1, 2, 3, or 4. In some embodiments, m is 0. In some embodiments, m is 1. In some embodiments, m is 2. In some embodiments, m is 3. In some embodiments, m is 4. In some embodiments, m is 0 or 1. In some embodiments, m is 0, 1, or 2.

In some embodiments, m is 0, 1, 2, or 3. In some embodiments, m is 1 or 2. In some embodiments, m is 1, 2, or 3. In some embodiments, m is 1, 2, 3, or 4. In some embodiments, m is 2 or 3. In some embodiments, m is 2, 3, or 4. In some embodiments, m is 3 or 4. In some embodiments, m is selected from the values represented in the compounds in Table 1.

As defined generally above, n is 0, 1, 2, 3, or 4. In some embodiments, n is 0. In some embodiments, n is 1. In some embodiments, n is 2. In some embodiments, n is 3. In some embodiments, n is 4. In some embodiments, n is 0 or 1. In some embodiments, n is 0, 1, or 2. In some embodiments, n is 0, 1, 2, or 3. In some embodiments, n is 1 or 2. In some embodiments, n is 1, 2, or 3. In some embodiments, n is 1, 2, 3, or 4. In some embodiments, n is 2 or 3. In some embodiments, n is 2, 3, or 4. In some embodiments, n is 3 or 4. In some embodiments, n is selected from the values represented in the compounds in Table 1.

As defined generally above, p is 0, 1, 2, 3, or 4. In some embodiments, p is 0. In some embodiments, p is 1. In some embodiments, p is 2. In some embodiments, p is 3. In some embodiments, p is 4. In some embodiments, p is 0 or 1. In some embodiments, p is 0, 1, or 2. In some embodiments, p is 0, 1, 2, or 3. In some embodiments, p is 1 or 2. In some embodiments, p is 1, 2, or 3. In some embodiments, p is 1, 2, 3, or 4. In some embodiments, p is 2 or 3. In some embodiments, p is 2, 3, or 4. In some embodiments, p is 3 or 4. In some embodiments, p is selected from the values represented in the compounds in Table 1.

As defined generally above, q^{A} is 0, 1, 2, 3, or 4. In some embodiments, q^{A} is 0. In some embodiments, q^{A} is 1. In some embodiments, q^{A} is 2. In some embodiments, q^{A} is 3. In some embodiments, q^{A} is 4. In some embodiments, q^{A} is 0 or 1. In some embodiments, q^{A} is 0, 1, or 2. In some embodiments, q^{A} is 0, 1, 2, or 3. In some embodiments, q^{A} is 1 or 2. In some embodiments, q^{A} is 1, 2, or 3. In some embodiments, q^{A} is 1, 2, 3, or 4. In some embodiments, q^{A} is 2 or 3. In some embodiments, q^{A} is 2, 3, or 4. In some embodiments, q^{A} is 3 or 4. In some embodiments, q^{A} is selected from the values represented in the compounds in Table 1.

As defined generally above, q^{B} is 0, 1, 2, 3, or 4. In some embodiments, q^{B} is 0. In some embodiments, q^{B} is 1. In some embodiments, q^{B} is 2. In some embodiments, q^{B} is 3. In some embodiments, q^{B} is 4. In some embodiments, q^{B} is 0 or 1. In some embodiments, q^{B} is 0, 1, or 2. In some embodiments, q^{B} is 0, 1, 2, or 3. In some embodiments, q^{B} is 1 or 2. In some embodiments, q^{B} is 1, 2, or 3. In some embodiments, q^{B} is 1, 2, 3, or 4. In some embodiments, q^{B} is 2 or 3. In some embodiments, q^{B} is 2, 3, or 4. In some embodiments, q^{B} is 3 or 4. In some embodiments, q^{B} is selected from the values represented in the compounds in Table 1.

As defined generally above, q^{C} is 0, 1, 2, 3, or 4. In some embodiments, q^{C} is 0. In some embodiments, q^{C} is 1. In some embodiments, q^{C} is 2. In some embodiments, q^{C} is 3. In some embodiments, q^{C} is 4. In some embodiments, q^{C} is 0 or 1. In some embodiments, q^{C} is 0, 1, or 2. In some embodiments, q^{C} is 0, 1, 2, or 3. In some embodiments, q^{C} is 1 or 2. In some embodiments, q^{C} is 1, 2, or 3. In some embodiments, q^{C} is 1, 2, 3, or 4. In some embodiments, q^{C} is 2 or 3. In some embodiments, q^{C} is 2, 3, or 4. In some embodiments, q^{C} is 3 or 4. In some embodiments, q^{C} is selected from the values represented in the compounds in Table 1.

As defined generally above, r is 0, 1, 2, 3, or 4. In some embodiments, r is 0. In some embodiments, r is 1. In some embodiments, r is 2. In some embodiments, r is 3. In some embodiments, r is 4. In some embodiments, r is 0 or 1. In some embodiments, r is 0, 1, or 2. In some embodiments, r is 0, 1, 2, or 3. In some embodiments, r is 1 or 2. In some embodiments, r is 1, 2, or 3. In some embodiments, r is 1, 2, 3, or 4. In some embodiments, r is 2 or 3. In some embodiments, r is 2, 3, or 4. In some embodiments, r is 3 or 4. In some embodiments, r is selected from the values represented in the compounds in Table 1.

As defined generally above, t is 0, 1, 2, 3, or 4. In some embodiments, t is 0. In some embodiments, t is 1. In some embodiments, t is 2. In some embodiments, t is 3. In some embodiments, t is 4. In some embodiments, t is 0 or 1. In some embodiments, t is 0, 1, or 2. In some embodiments, t is 0, 1, 2, or 3. In some embodiments, t is 1 or 2. In some embodiments, t is 1, 2, or 3. In some embodiments, t is 1, 2, 3, or 4. In some embodiments, t is 2 or 3. In some embodiments, t is 2, 3, or 4. In some embodiments, t is 3 or 4. In some embodiments, t is selected from the values represented in the compounds in Table 1.

Examples of compounds described herein include those listed in the Tables and exemplification herein, or a pharmaceutically acceptable salt, stereoisomer, or mixture of stereoisomers thereof. In some embodiments, the present disclosure comprises a compound selected from those depicted in Table 1, below, or a pharmaceutically acceptable salt, stereoisomer, or mixture of stereoisomers thereof. In some embodiments, the present disclosure provides a compound set forth in Table 1, below, or a pharmaceutically acceptable salt thereof. In some embodiments, the present disclosure provides a compound set forth in Table 1, below.

TABLE 1
Representative Compounds with Bioactivity Data.

| | | Biochemical | Cell |
|---|---|---|---|
| | | CDK2/CyclinE | nanoBRET |
| Example | Structure | Caliper IC50 | IC50 |
| 1 | 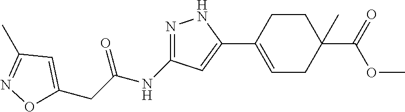 | | |
| 2 | 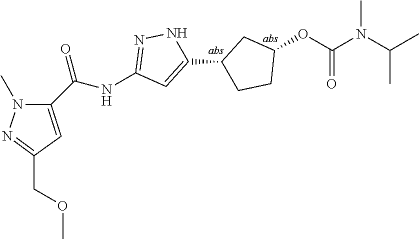 [SMILES: COCc1cc(C(=O)Nc2cc([C@H]3CC[C@@H](OC(=O)N(C)C(C)C)C3)[nH]n2)n(C)n1] | A | B |
| 3 | 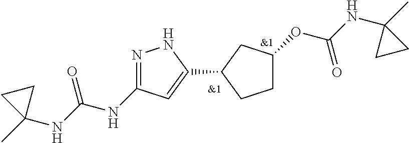 [SMILES: CC1(NC(=O)Nc2cc([C@H]3CC[C@@H](OC(=O)NC4(C)CC4)C3)[nH]n2)CC1] | B | D |
| 4 | 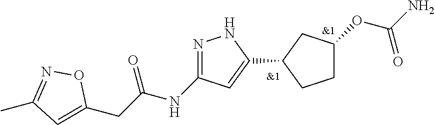 [SMILES: Cc1cc(CC(=O)Nc2cc([C@H]3CC[C@@H](OC(N)=O)C3)[nH]n2)on1] | C | D |
| 5 | 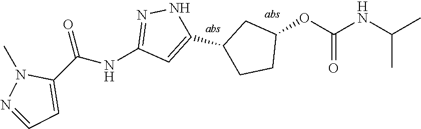 [SMILES: CC(C)NC(=O)O[C@@H]1CC[C@H](c2cc(NC(=O)c3ccnn3C)n[nH]2)C1] | A | A |
| 6 | 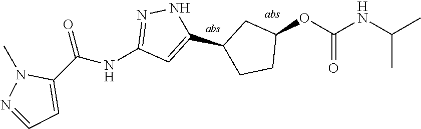 [SMILES: CC(C)NC(=O)O[C@H]1CC[C@@H](c2cc(NC(=O)c3ccnn3C)n[nH]2)C1] | A | B |
| 7 | 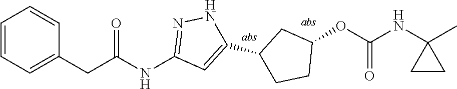 [SMILES: CC1(NC(=O)O[C@@H]2CC[C@H](c3cc(NC(=O)Cc4ccccc4)n[nH]3)C2)CC1] | A | A |
| 8 | 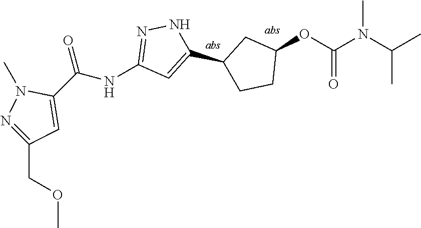 [SMILES: COCc1cc(C(=O)Nc2cc([C@@H]3CC[C@H](OC(=O)N(C)C(C)C)C3)[nH]n2)n(C)n1] | A | B |
| 9 | 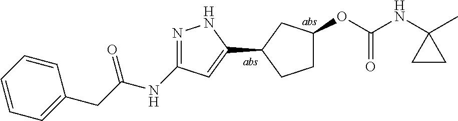 [SMILES: CC1(NC(=O)O[C@H]2CC[C@@H](c3cc(NC(=O)Cc4ccccc4)n[nH]3)C2)CC1] | A | A |
| 10 | 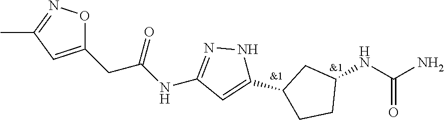 [SMILES: Cc1cc(CC(=O)Nc2cc([C@H]3CC[C@@H](NC(N)=O)C3)[nH]n2)on1] | C | |
| 11 | 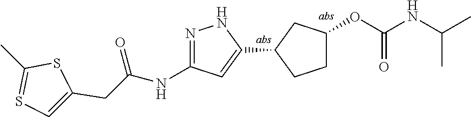 | A | B |
| 12 | 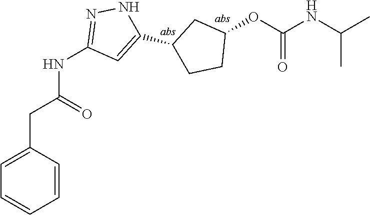 [SMILES: CC(C)NC(=O)O[C@@H]1CC[C@H](c2cc(NC(=O)Cc3ccccc3)n[nH]2)C1] | A | B |
| 13 | 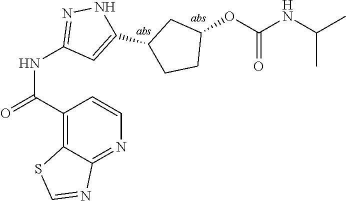 [SMILES: CC(C)NC(=O)O[C@@H]1CC[C@H](c2cc(NC(=O)c3ccnc4ncsc34)n[nH]2)C1] | A | A |
| 14 | 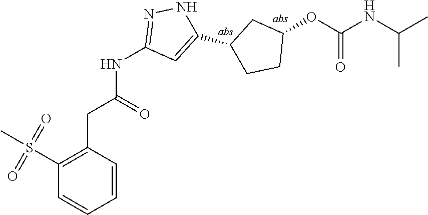 [SMILES: CC(C)NC(=O)O[C@@H]1CC[C@H](c2cc(NC(=O)Cc3ccccc3S(C)(=O)=O)n[nH]2)C1] | A | B |
| 15 | 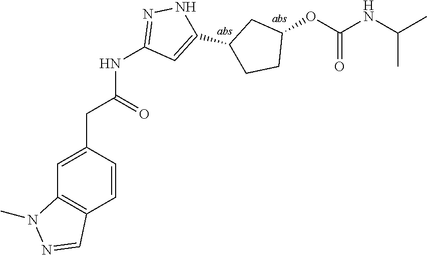 [SMILES: CC(C)NC(=O)O[C@@H]1CC[C@H](c2cc(NC(=O)Cc3ccc4cnn(C)c4c3)n[nH]2)C1] | A | C |
| 16 | 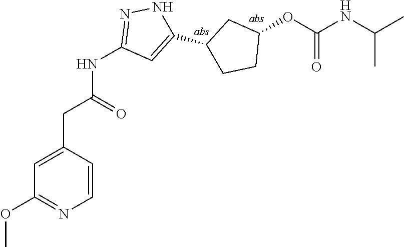 [SMILES: COc1cc(CC(=O)Nc2cc([C@H]3CC[C@@H](OC(=O)NC(C)C)C3)[nH]n2)ccn1] | A | B |
| 17 | 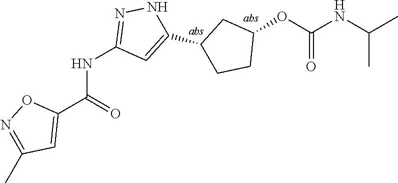 [SMILES: Cc1cc(C(=O)Nc2cc([C@H]3CC[C@@H](OC(=O)NC(C)C)C3)[nH]n2)on1] | A | D |
| 18 | 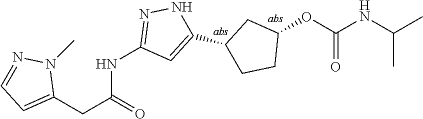 [SMILES: CC(C)NC(=O)O[C@@H]1CC[C@H](c2cc(NC(=O)Cc3ccnn3C)n[nH]2)C1] | A | C |
| 19 | 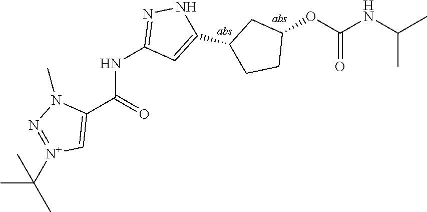 [SMILES: CC(C)NC(=O)O[C@@H]1CC[C@H](c2cc(NC(=O)c3c[n+](C(C)(C)C)nn3C)n[nH]2)C1] | C | D |
| 20 | 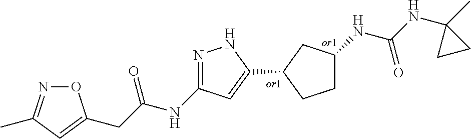 [SMILES: Cc1cc(CC(=O)Nc2cc([C@H]3CC[C@@H](NC(=O)NC4(C)CC4)C3)[nH]n2)on1] | D | D |
| 21 | 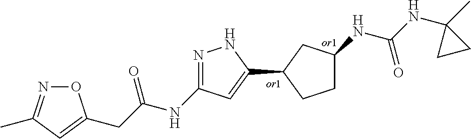 [SMILES: Cc1cc(CC(=O)Nc2cc([C@@H]3CC[C@H](NC(=O)NC4(C)CC4)C3)[nH]n2)on1] | A | D |
| 22 | 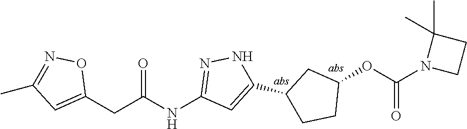 [SMILES: Cc1cc(CC(=O)Nc2cc([C@H]3CC[C@@H](OC(=O)N4CCC4(C)C)C3)[nH]n2)on1] | A | A |
| 23 | 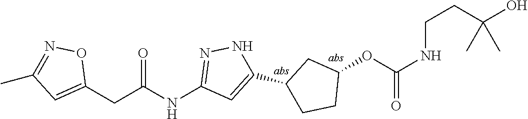 [SMILES: Cc1cc(CC(=O)Nc2cc([C@H]3CC[C@@H](OC(=O)NCCC(C)(C)O)C3)[nH]n2)on1] | B | D |
| 24 | 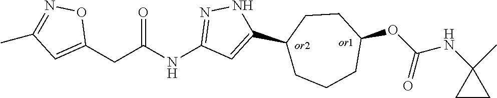 | B | C |
| 25 | 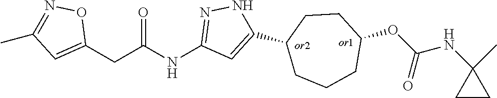 | A | B |
| 26 | 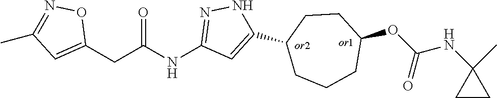 | D | |
| 27 | 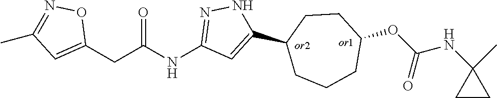 | D | |
| 28 | 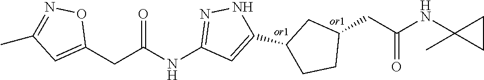 [SMILES: Cc1cc(CC(=O)Nc2cc([C@H]3CC[C@@H](CC(=O)NC4(C)CC4)C3)[nH]n2)on1] | D | D |
| 29 | 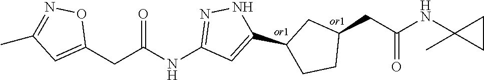 [SMILES: Cc1cc(CC(=O)Nc2cc([C@@H]3CC[C@H](CC(=O)NC4(C)CC4)C3)[nH]n2)on1] | D | D |
| 30 | 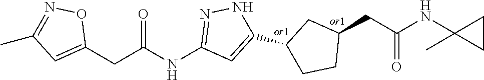 [SMILES: Cc1cc(CC(=O)Nc2cc([C@H]3CC[C@H](CC(=O)NC4(C)CC4)C3)[nH]n2)on1] | D | D |
| 31 | 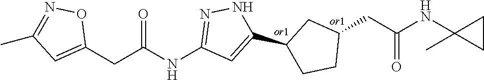 [SMILES: Cc1cc(CC(=O)Nc2cc([C@@H]3CC[C@@H](CC(=O)NC4(C)CC4)C3)[nH]n2)on1] | D | D |
| 32 | 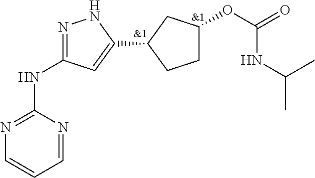 [SMILES: CC(C)NC(=O)O[C@@H]1CC[C@H](c2cc(Nc3ncccn3)n[nH]2)C1] | B | D |
| 33 | 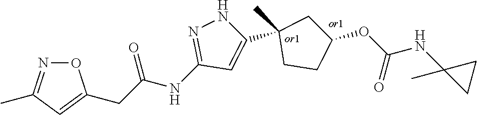 [SMILES: Cc1cc(CC(=O)Nc2cc([C@@]3(C)CC[C@@H](OC(=O)NC4(C)CC4)C3)[nH]n2)on1] | D | D |
| 34 | 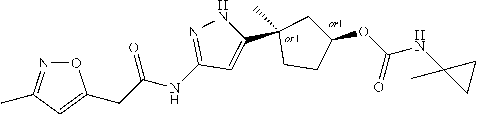 [SMILES: Cc1cc(CC(=O)Nc2cc([C@]3(C)CC[C@H](OC(=O)NC4(C)CC4)C3)[nH]n2)on1] | D | D |
| 35 | 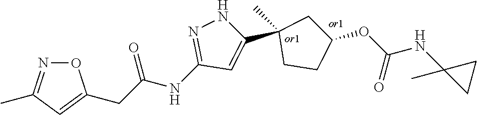 [SMILES: Cc1cc(CC(=O)Nc2cc([C@]3(C)CC[C@@H](OC(=O)NC4(C)CC4)C3)[nH]n2)on1] | D | D |
| 36 | 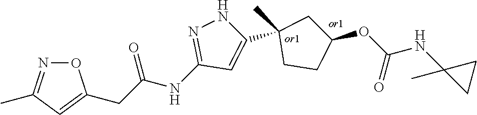 [SMILES: Cc1cc(CC(=O)Nc2cc([C@@]3(C)CC[C@H](OC(=O)NC4(C)CC4)C3)[nH]n2)on1] | D | D |
| 37 | 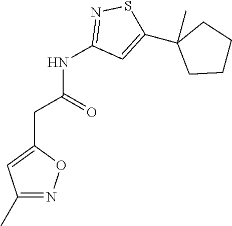 [SMILES: Cc1cc(CC(=O)Nc2cc(C3(C)CCCC3)sn2)on1] | D | |
| 38 | 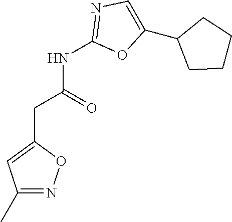 [SMILES: Cc1cc(CC(=O)Nc2ncc(C3CCCC3)o2)on1] | D | |
| 39 | 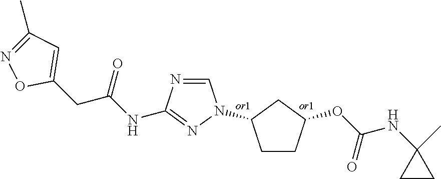 [SMILES: Cc1cc(CC(=O)Nc2ncn([C@H]3CC[C@@H](OC(=O)NC4(C)CC4)C3)n2)on1] | D | D |
| 40 | 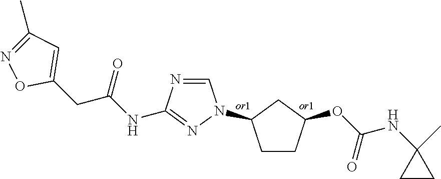 [SMILES: Cc1cc(CC(=O)Nc2ncn([C@@H]3CC[C@H](OC(=O)NC4(C)CC4)C3)n2)on1] | D | D |
| 41 | 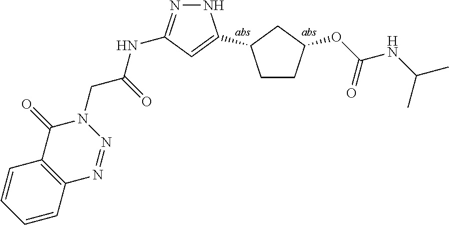 [SMILES: CC(C)NC(=O)O[C@@H]1CC[C@H](c2cc(NC(=O)Cn3nnc4ccccc4c3=O)n[nH]2)C1] | A | D |
| 42 | 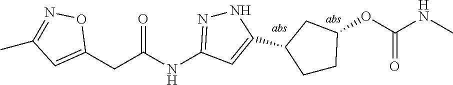 | B | D |
| 43 | 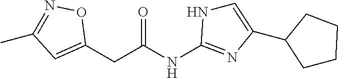 [SMILES: Cc1cc(CC(=O)Nc2nc(C3CCCC3)c[nH]2)on1] | D | |
| 44 | 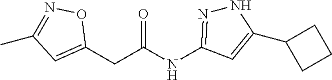 [SMILES: Cc1cc(CC(=O)Nc2cc(C3CCC3)[nH]n2)on1] | D | |
| 45 | 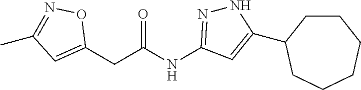 [SMILES: Cc1cc(CC(=O)Nc2cc(C3CCCCCC3)[nH]n2)on1] | D | |
| 46 | 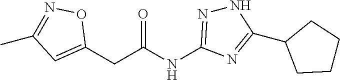 [SMILES: Cc1cc(CC(=O)Nc2n[nH]c(C3CCCC3)n2)on1] | D | |
| 47 | 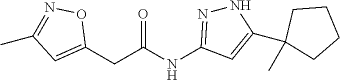 [SMILES: Cc1cc(CC(=O)Nc2cc(C3(C)CCCC3)[nH]n2)on1] | D | |
| 48 | 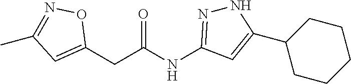 [SMILES: Cc1cc(CC(=O)Nc2cc(C3CCCCC3)[nH]n2)on1] | D | |
| 49 | 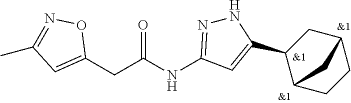 [SMILES: Cc1cc(CC(=O)Nc2cc([C@H]3C[C@@H]4CC[C@H]3C4)[nH]n2)on1] | D | |
| 50 | 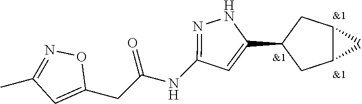 [SMILES: Cc1cc(CC(=O)Nc2cc([C@H]3C[C@@H]4C[C@@H]4C3)[nH]n2)on1] | D | |
| 51 | 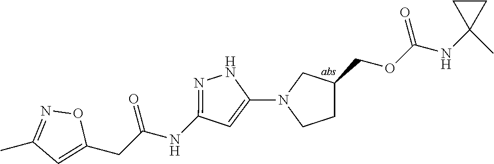 | D | |
| 52 | 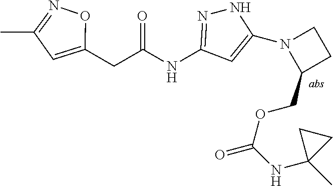 [SMILES: Cc1cc(CC(=O)Nc2cc(N3CC[C@H]3COC(=O)NC3(C)CC3)[nH]n2)on1] | D | |
| 53 | 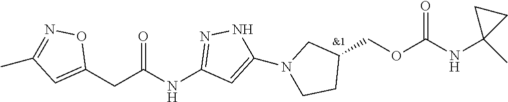 [SMILES: Cc1cc(CC(=O)Nc2cc(N3CC[C@@H](COC(=O)NC4(C)CC4)C3)[nH]n2)on1] | D | |
| 54 | 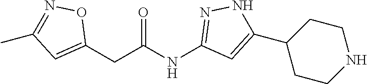 [SMILES: Cc1cc(CC(=O)Nc2cc(C3CCNCC3)[nH]n2)on1] | D | |
| 55 | 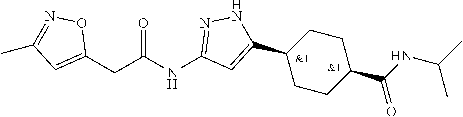 [SMILES: Cc1cc(CC(=O)Nc2cc([C@H]3CC[C@@H](C(=O)NC(C)C)CC3)[nH]n2)on1] | D | D |
| 56 | 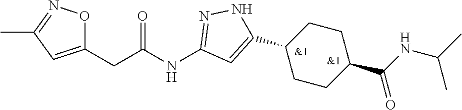 [SMILES: Cc1cc(CC(=O)Nc2cc([C@H]3CC[C@H](C(=O)NC(C)C)CC3)[nH]n2)on1] | D | D |
| 57 | 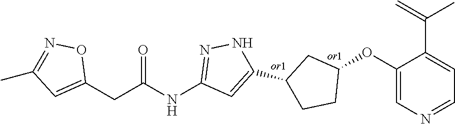 [SMILES: C=C(C)c1ccncc1O[C@@H]1CC[C@H](c2cc(NC(=O)Cc3cc(C)no3)n[nH]2)C1] | A | A |
| 58 | 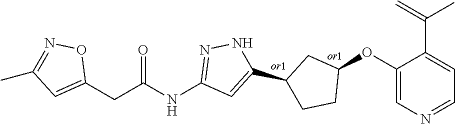 [SMILES: C=C(C)c1ccncc1O[C@H]1CC[C@@H](c2cc(NC(=O)Cc3cc(C)no3)n[nH]2)C1] | A | A |
| 59 | 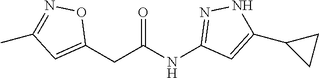 [SMILES: Cc1cc(CC(=O)Nc2cc(C3CC3)[nH]n2)on1] | D | |
| 60 | 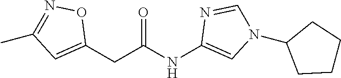 [SMILES: Cc1cc(CC(=O)Nc2cn(C3CCCC3)cn2)on1] | D | |
| 61 | 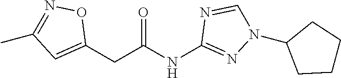 [SMILES: Cc1cc(CC(=O)Nc2ncn(C3CCCC3)n2)on1] | D | |
| 62 | 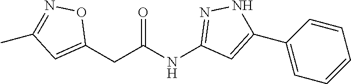 [SMILES: Cc1cc(CC(=O)Nc2cc(-c3ccccc3)[nH]n2)on1] | D | |
| 63 | 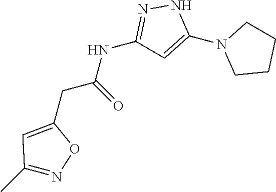 [SMILES: Cc1cc(CC(=O)Nc2cc(N3CCCC3)[nH]n2)on1] | | |
| 64 | 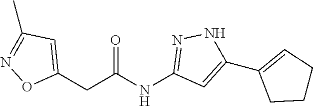 [SMILES: Cc1cc(CC(=O)Nc2cc(C3=CCCC3)[nH]n2)on1] | | |
| 65 | 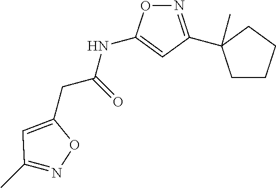 [SMILES: Cc1cc(CC(=O)Nc2cc(C3(C)CCCC3)no2)on1] | | |
| 66 | 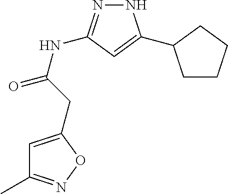 [SMILES: Cc1cc(CC(=O)Nc2cc(C3CCCC3)[nH]n2)on1] | D | |
| 67 | 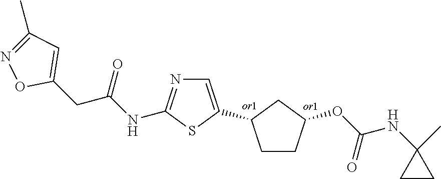 [SMILES: Cc1cc(CC(=O)Nc2ncc([C@H]3CC[C@@H](OC(=O)NC4(C)CC4)C3)s2)on1] | | |
| 68 | 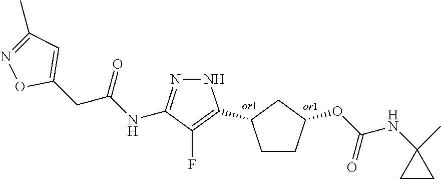 [SMILES: Cc1cc(CC(=O)Nc2n[nH]c([C@H]3CC[C@@H](OC(=O)NC4(C)CC4)C3)c2F)on1] | | |
| 69 | 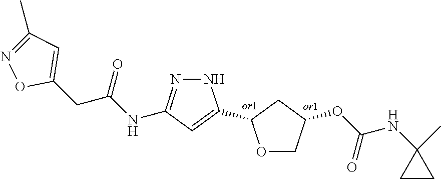 [SMILES: Cc1cc(CC(=O)Nc2cc([C@@H]3C[C@H](OC(=O)NC4(C)CC4)CO3)[nH]n2)on1] | A | |
| 70 | 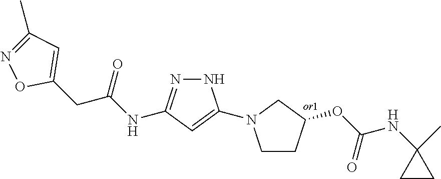 [SMILES: Cc1cc(CC(=O)Nc2cc(N3CC[C@@H](OC(=O)NC4(C)CC4)C3)[nH]n2)on1] | | |
| 71 | 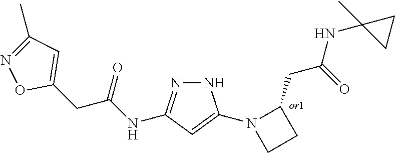 [SMILES: Cc1cc(CC(=O)Nc2cc(N3CC[C@H]3CC(=O)NC3(C)CC3)[nH]n2)on1] | | |
| 72 | 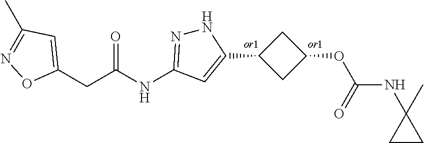 [SMILES: Cc1cc(CC(=O)Nc2cc([C@H]3C[C@@H](OC(=O)NC4(C)CC4)C3)[nH]n2)on1] | | |
| 73 | 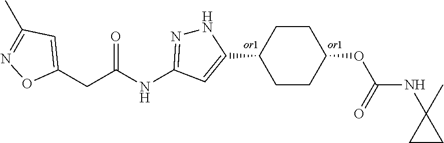 [SMILES: Cc1cc(CC(=O)Nc2cc([C@H]3CC[C@@H](OC(=O)NC4(C)CC4)CC3)[nH]n2)on1] | | |
| 74 | 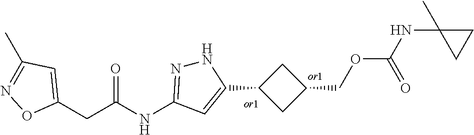 [SMILES: Cc1cc(CC(=O)Nc2cc([C@H]3C[C@@H](COC(=O)NC4(C)CC4)C3)[nH]n2)on1] | | |
| 75 | 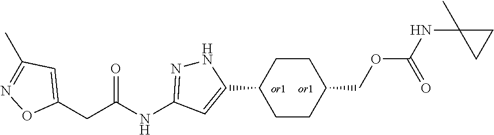 [SMILES: Cc1cc(CC(=O)Nc2cc([C@H]3CC[C@@H](COC(=O)NC4(C)CC4)CC3)[nH]n2)on1] | | |
| 76 | 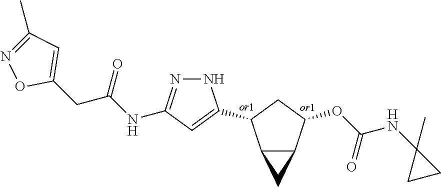 [SMILES: Cc1cc(CC(=O)Nc2cc([C@@H]3C[C@H](OC(=O)NC4(C)CC4)[C@H]4C[C@H]43)[nH]n2)on1] | | |
| 77 | 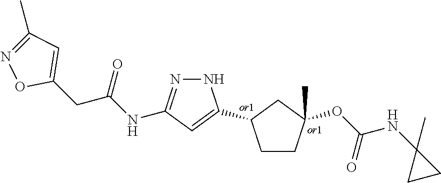 [SMILES: Cc1cc(CC(=O)Nc2cc([C@H]3CC[C@@](C)(OC(=O)NC4(C)CC4)C3)[nH]n2)on1] | | |
| 78 | 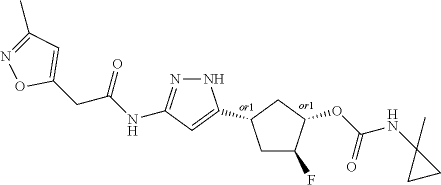 [SMILES: Cc1cc(CC(=O)Nc2cc([C@@H]3C[C@H](OC(=O)NC4(C)CC4)[C@@H](F)C3)[nH]n2)on1] | | |
| 79 | 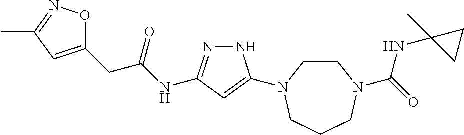 [SMILES: Cc1cc(CC(=O)Nc2cc(N3CCCN(C(=O)NC4(C)CC4)CC3)[nH]n2)on1] | | |
| 80 | 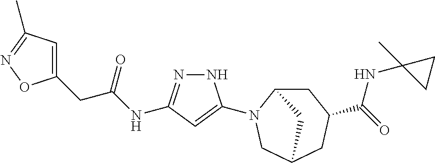 [SMILES: Cc1cc(CC(=O)Nc2cc(N3C[C@@H]4C[C@@H](C(=O)NC5(C)CC5)C[C@H]3C4)[nH]n2)on1] | | |
| 81 | 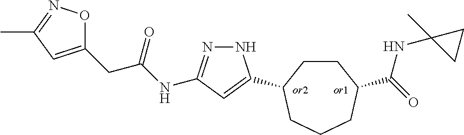 [SMILES: Cc1cc(CC(=O)Nc2cc([C@H]3CCC[C@@H](C(=O)NC4(C)CC4)CC3)[nH]n2)on1] | | |
| 82 | 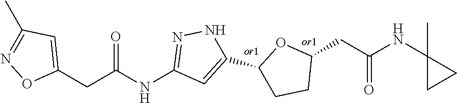 [SMILES: Cc1cc(CC(=O)Nc2cc([C@H]3CC[C@@H](CC(=O)NC4(C)CC4)O3)[nH]n2)on1] | | |
| 83 | 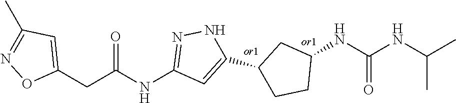 [SMILES: Cc1cc(CC(=O)Nc2cc([C@H]3CC[C@@H](NC(=O)NC(C)C)C3)[nH]n2)on1] | D | D |
| 84 | 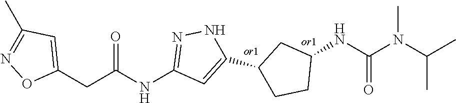 [SMILES: Cc1cc(CC(=O)Nc2cc([C@H]3CC[C@@H](NC(=O)N(C)C(C)C)C3)[nH]n2)on1] | | |
| 85 | 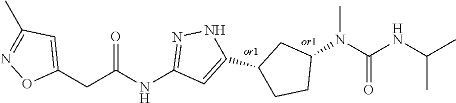 [SMILES: Cc1cc(CC(=O)Nc2cc([C@H]3CC[C@@H](N(C)C(=O)NC(C)C)C3)[nH]n2)on1] | | |
| 86 | 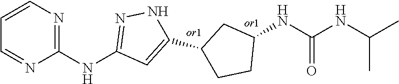 [SMILES: CC(C)NC(=O)N[C@@H]1CC[C@H](c2cc(Nc3ncccn3)n[nH]2)C1] | D | |
| 87 | 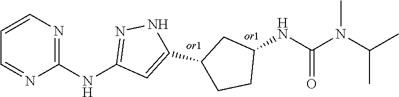 [SMILES: CC(C)N(C)C(=O)N[C@@H]1CC[C@H](c2cc(Nc3ncccn3)n[nH]2)C1] | | |
| 88 | 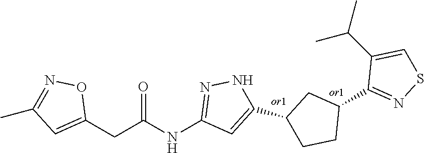 [SMILES: Cc1cc(CC(=O)Nc2cc([C@H]3CC[C@@H](c4nscc4C(C)C)C3)[nH]n2)on1] | | |
| 89 | 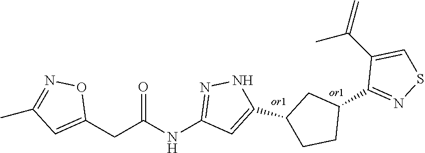 [SMILES: C=C(C)c1csnc1[C@@H]1CC[C@H](c2cc(NC(=O)Cc3cc(C)no3)n[nH]2)C1] | | |
| 90 | 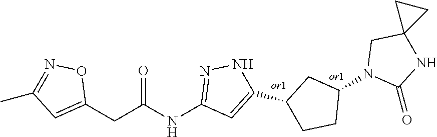 [SMILES: Cc1cc(CC(=O)Nc2cc([C@H]3CC[C@@H](N4CC5(CC5)NC4=O)C3)[nH]n2)on1] | C | |
| 91 | 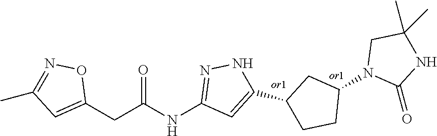 | A | D |
| 92 | 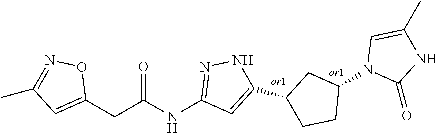 | C | |
| 93 | 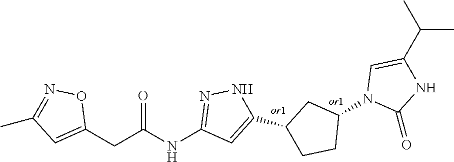 [SMILES: Cc1cc(CC(=O)Nc2cc([C@H]3CC[C@@H](n4cc(C(C)C)[nH]c4=O)C3)[nH]n2)on1] | D | |
| 94 | 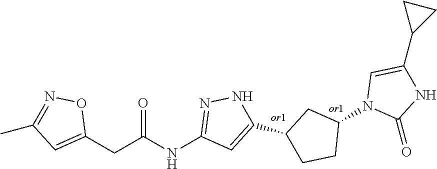 [SMILES: Cc1cc(CC(=O)Nc2cc([C@H]3CC[C@@H](n4cc(C5CC5)[nH]c4=O)C3)[nH]n2)on1] | | |
| 95 | 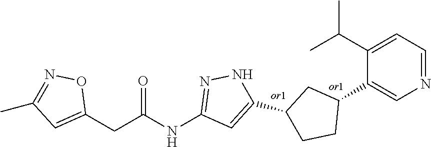 [SMILES: Cc1cc(CC(=O)Nc2cc([C@H]3CC[C@@H](c4cnccc4C(C)C)C3)[nH]n2)on1] | | |
| 96 | 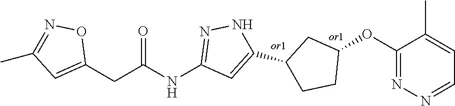 [SMILES: Cc1cc(CC(=O)Nc2cc([C@H]3CC[C@@H](Oc4nnccc4C)C3)[nH]n2)on1] | | |
| 97 | 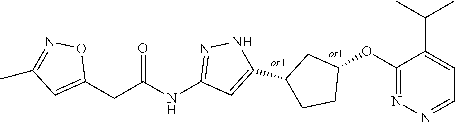 [SMILES: Cc1cc(CC(=O)Nc2cc([C@H]3CC[C@@H](Oc4nnccc4C(C)C)C3)[nH]n2)on1] | A | |
| 98 | 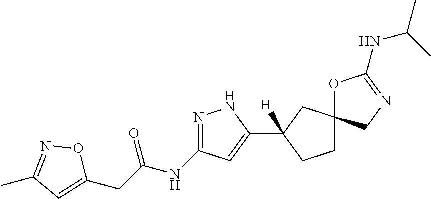 [SMILES: Cc1cc(CC(=O)Nc2cc([C@H]3CC[C@]4(CN=C(NC(C)C)O4)C3)[nH]n2)on1] | | |
| 99 | 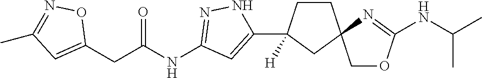 [SMILES: Cc1cc(CC(=O)Nc2cc([C@H]3CC[C@]4(COC(NC(C)C)=N4)C3)[nH]n2)on1] | | |
| 100 | 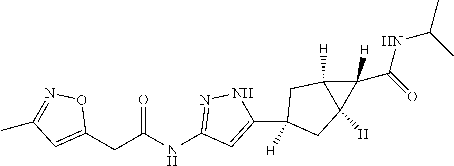 [SMILES: Cc1cc(CC(=O)Nc2cc([C@H]3C[C@@H]4[C@H](C3)[C@H]4C(=O)NC(C)C)[nH]n2)on1] | | |
| 101 | 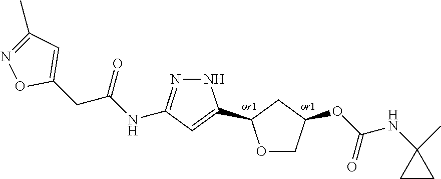 [SMILES: Cc1cc(CC(=O)Nc2cc([C@H]3C[C@@H](OC(=O)NC4(C)CC4)CO3)[nH]n2)on1] | A | |
| 102 | 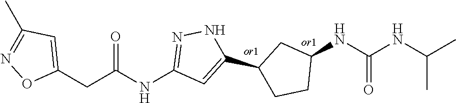 [SMILES: Cc1cc(CC(=O)Nc2cc([C@@H]3CC[C@H](NC(=O)NC(C)C)C3)[nH]n2)on1] | D | D |
| 103 | 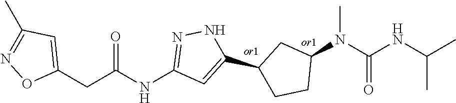 [SMILES: Cc1cc(CC(=O)Nc2cc([C@@H]3CC[C@H](N(C)C(=O)NC(C)C)C3)[nH]n2)on1] | D | |
| 104 | 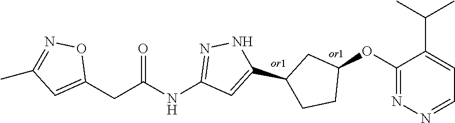 [SMILES: Cc1cc(CC(=O)Nc2cc([C@@H]3CC[C@H](Oc4nnccc4C(C)C)C3)[nH]n2)on1] | A | |
| 105 | 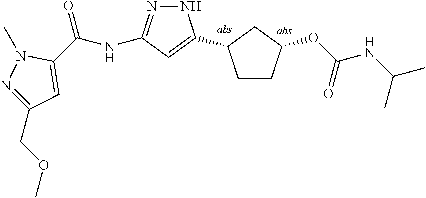 [SMILES: COCc1cc(C(=O)Nc2cc([C@H]3CC[C@@H](OC(=O)NC(C)C)C3)[nH]n2)n(C)n1] | A | C |
| 106 | 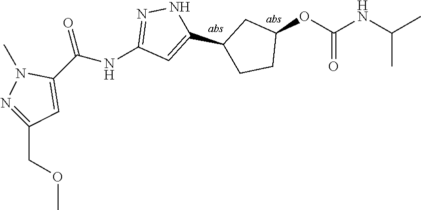 [SMILES: COCc1cc(C(=O)Nc2cc([C@@H]3CC[C@H](OC(=O)NC(C)C)C3)[nH]n2)n(C)n1] | A | C |
| 107 | 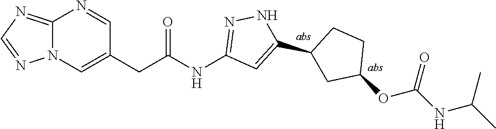 [SMILES: CC(C)NC(=O)O[C@@H]1CC[C@H](c2cc(NC(=O)Cc3cnc4ncnn4c3)n[nH]2)C1] | C | D |
| 108 | 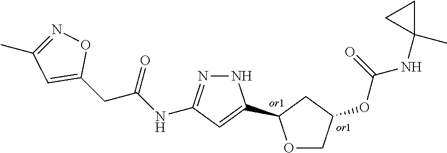 | D | |
| 109 | 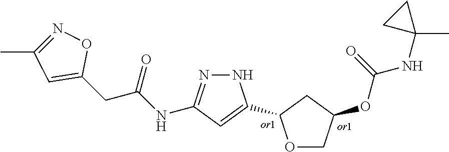 | D | |
| 110 | 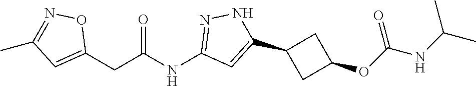 [SMILES: Cc1cc(CC(=O)Nc2cc([C@H]3C[C@@H](OC(=O)NC(C)C)C3)[nH]n2)on1] | A | D |
| 111 | 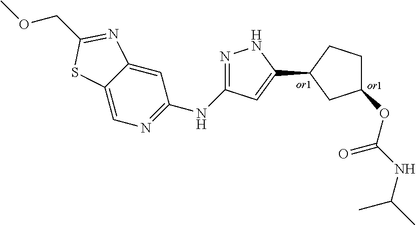 [SMILES: COCc1nc2cc(Nc3cc([C@H]4CC[C@@H](OC(=O)NC(C)C)C4)[nH]n3)ncc2s1] | C | |
| 112 | 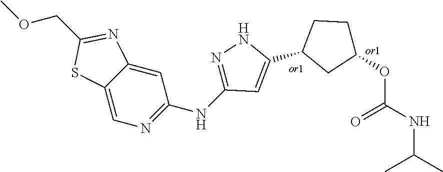 [SMILES: COCc1nc2cc(Nc3cc([C@@H]4CC[C@H](OC(=O)NC(C)C)C4)[nH]n3)ncc2s1] | B | |
| 113 | 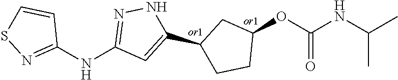 [SMILES: CC(C)NC(=O)O[C@H]1CC[C@@H](c2cc(Nc3ccsn3)n[nH]2)C1] | D | |
| 114 | 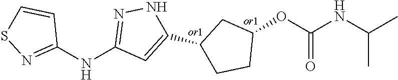 [SMILES: CC(C)NC(=O)O[C@@H]1CC[C@H](c2cc(Nc3ccsn3)n[nH]2)C1] | D | |
| 115 | 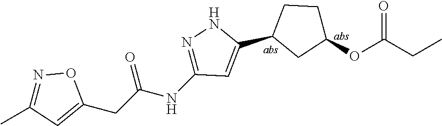 [SMILES: CCC(=O)O[C@@H]1CC[C@H](c2cc(NC(=O)Cc3cc(C)no3)n[nH]2)C1] | B | D |
| 116 | 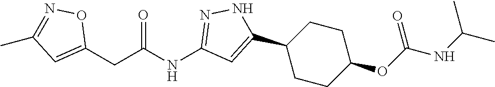 [SMILES: Cc1cc(CC(=O)Nc2cc([C@H]3CC[C@@H](OC(=O)NC(C)C)CC3)[nH]n2)on1] | A | C |
| 117 | 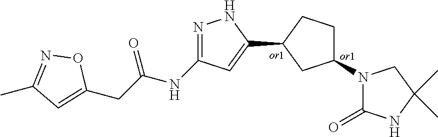 | A | D |
| 118 | 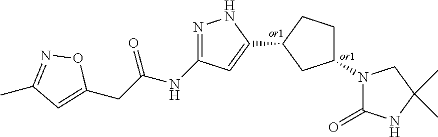 | B | |
| 119 | 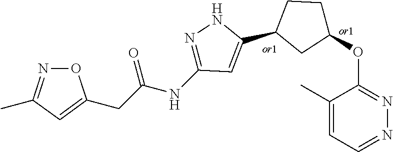 [SMILES: Cc1cc(CC(=O)Nc2cc([C@H]3CC[C@@H](Oc4nnccc4C)C3)[nH]n2)on1] | A | C |
| 120 | 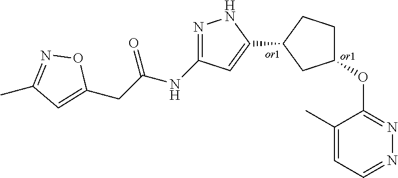 [SMILES: Cc1cc(CC(=O)Nc2cc([C@@H]3CC[C@H](Oc4nnccc4C)C3)[nH]n2)on1] | A | |
| 121 | 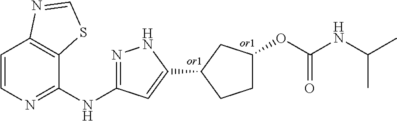 [SMILES: CC(C)NC(=O)O[C@@H]1CC[C@H](c2cc(Nc3nccc4ncsc34)n[nH]2)C1] | A | |
| 122 | 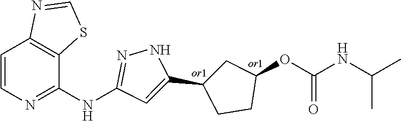 [SMILES: CC(C)NC(=O)O[C@H]1CC[C@@H](c2cc(Nc3nccc4ncsc34)n[nH]2)C1] | A | |
| 123 | 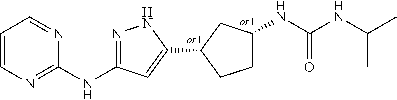 [SMILES: CC(C)NC(=O)N[C@@H]1CC[C@H](c2cc(Nc3ncccn3)n[nH]2)C1] | D | |
| 124 | 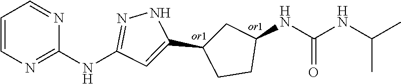 [SMILES: CC(C)NC(=O)N[C@H]1CC[C@@H](c2cc(Nc3ncccn3)n[nH]2)C1] | D | |
| 125 | 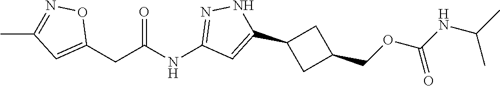 [SMILES: Cc1cc(CC(=O)Nc2cc([C@H]3C[C@@H](COC(=O)NC(C)C)C3)[nH]n2)on1] | A | |
| 126 | 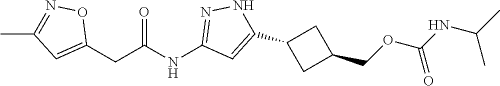 [SMILES: Cc1cc(CC(=O)Nc2cc([C@H]3C[C@H](COC(=O)NC(C)C)C3)[nH]n2)on1] | | |
| 127 | 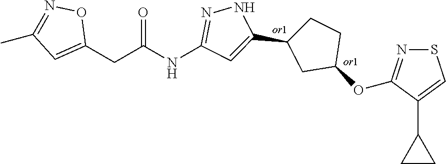 [SMILES: Cc1cc(CC(=O)Nc2cc([C@H]3CC[C@@H](Oc4nscc4C4CC4)C3)[nH]n2)on1] | A | |
| 128 | 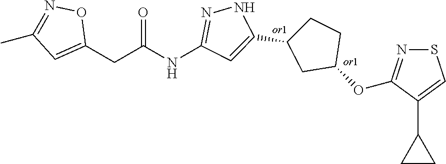 [SMILES: Cc1cc(CC(=O)Nc2cc([C@@H]3CC[C@H](Oc4nscc4C4CC4)C3)[nH]n2)on1] | A | |
| 129 | 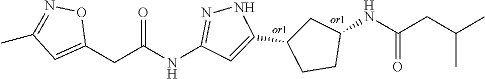 [SMILES: Cc1cc(CC(=O)Nc2cc([C@H]3CC[C@@H](NC(=O)CC(C)C)C3)[nH]n2)on1] | D | |
| 130 | 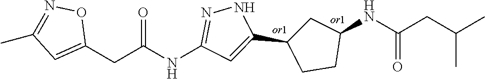 [SMILES: Cc1cc(CC(=O)Nc2cc([C@@H]3CC[C@H](NC(=O)CC(C)C)C3)[nH]n2)on1] | D | |
| 131 | 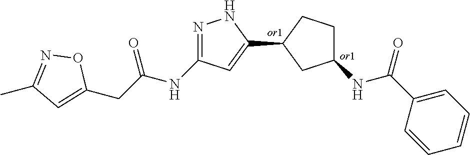 [SMILES: Cc1cc(CC(=O)Nc2cc([C@H]3CC[C@@H](NC(=O)c4ccccc4)C3)[nH]n2)on1] | D | |
| 132 | 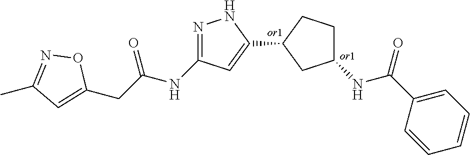 [SMILES: Cc1cc(CC(=O)Nc2cc([C@@H]3CC[C@H](NC(=O)c4ccccc4)C3)[nH]n2)on1] | D | |
| 133 | 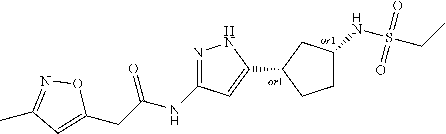 [SMILES: CCS(=O)(=O)N[C@@H]1CC[C@H](c2cc(NC(=O)Cc3cc(C)no3)n[nH]2)C1] | D | |
| 134 | 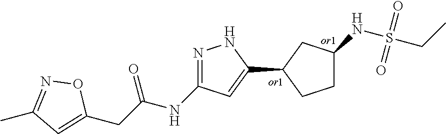 [SMILES: CCS(=O)(=O)N[C@H]1CC[C@@H](c2cc(NC(=O)Cc3cc(C)no3)n[nH]2)C1] | D | |
| 135 | 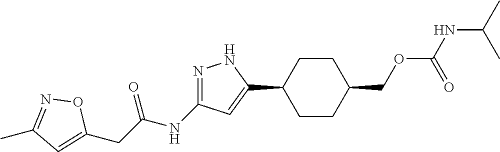 [SMILES: Cc1cc(CC(=O)Nc2cc([C@H]3CC[C@@H](COC(=O)NC(C)C)CC3)[nH]n2)on1] | D | |
| 136 | 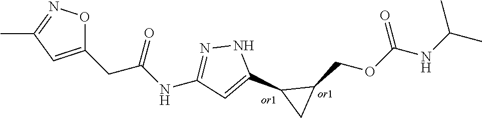 [SMILES: Cc1cc(CC(=O)Nc2cc([C@@H]3C[C@@H]3COC(=O)NC(C)C)[nH]n2)on1] | D | |
| 137 | 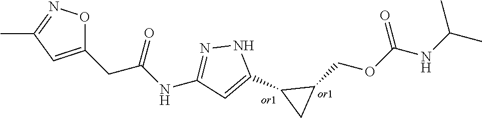 [SMILES: Cc1cc(CC(=O)Nc2cc([C@H]3C[C@H]3COC(=O)NC(C)C)[nH]n2)on1] | D | |
| 138 | 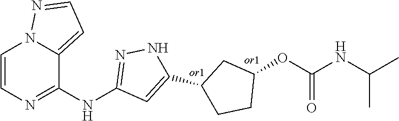 [SMILES: CC(C)NC(=O)O[C@@H]1CC[C@H](c2cc(Nc3nccn4nccc34)n[nH]2)C1] | A | |
| 139 | 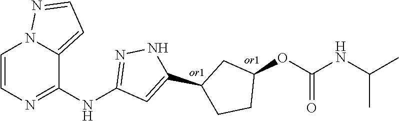 [SMILES: CC(C)NC(=O)O[C@H]1CC[C@@H](c2cc(Nc3nccn4nccc34)n[nH]2)C1] | A | |
| 140 | 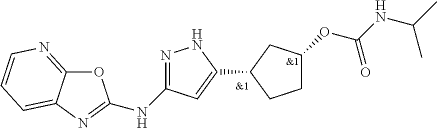 [SMILES: CC(C)NC(=O)O[C@@H]1CC[C@H](c2cc(Nc3nc4cccnc4o3)n[nH]2)C1] | A | |
| 141 | 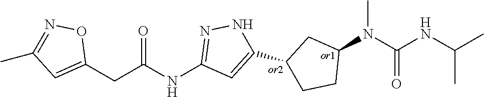 [SMILES: Cc1cc(CC(=O)Nc2cc([C@H]3CC[C@H](N(C)C(=O)NC(C)C)C3)[nH]n2)on1] | D | |
| 142 | 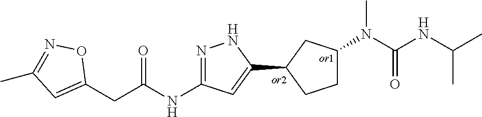 [SMILES: Cc1cc(CC(=O)Nc2cc([C@@H]3CC[C@@H](N(C)C(=O)NC(C)C)C3)[nH]n2)on1] | D | |
| 143 | 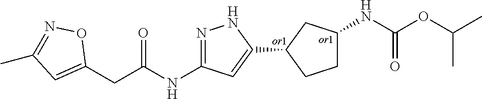 [SMILES: Cc1cc(CC(=O)Nc2cc([C@H]3CC[C@@H](NC(=O)OC(C)C)C3)[nH]n2)on1] | D | |
| 144 | 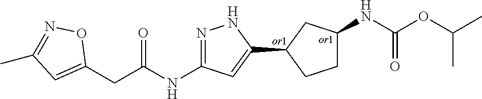 [SMILES: Cc1cc(CC(=O)Nc2cc([C@@H]3CC[C@H](NC(=O)OC(C)C)C3)[nH]n2)on1] | D | |
| 145 | 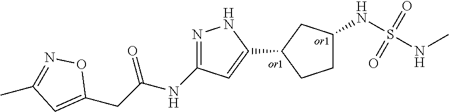 | D | |
| 146 | 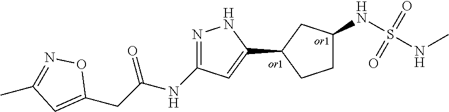 | D | |
| 147 | 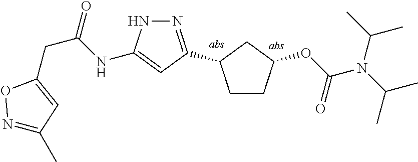 [SMILES: Cc1cc(CC(=O)Nc2cc([C@H]3CC[C@@H](OC(=O)N(C(C)C)C(C)C)C3)n[nH]2)on1] | A | |
| 148 | 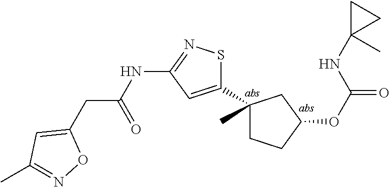 [SMILES: Cc1cc(CC(=O)Nc2cc([C@@]3(C)CC[C@@H](OC(=O)NC4(C)CC4)C3)sn2)on1] | | |
| 149 | 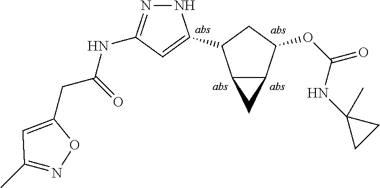 | | |
| 150 | 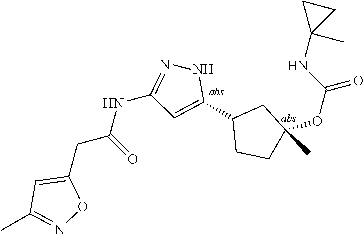 | | |
| 151 | 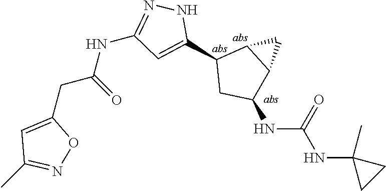 [SMILES: Cc1cc(CC(=O)Nc2cc([C@@H]3C[C@H](NC(=O)NC4(C)CC4)[C@H]4C[C@H]43)[nH]n2)on1] | | |
| 152 | 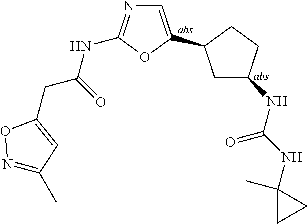 [SMILES: Cc1cc(CC(=O)Nc2ncc([C@H]3CC[C@@H](NC(=O)NC4(C)CC4)C3)o2)on1] | | |
| 153 | 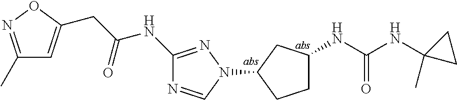 [SMILES: Cc1cc(CC(=O)Nc2ncn([C@H]3CC[C@@H](NC(=O)NC4(C)CC4)C3)n2)on1] | | |
| 154 | 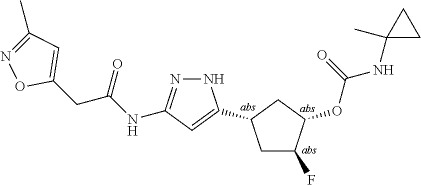 [SMILES: Cc1cc(CC(=O)Nc2cc([C@@H]3C[C@H](OC(=O)NC4(C)CC4)[C@@H](F)C3)[nH]n2)on1] | | |
| 155 | 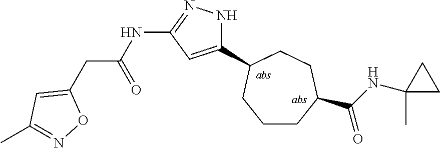 [SMILES: Cc1cc(CC(=O)Nc2cc([C@@H]3CCC[C@H](C(=O)NC4(C)CC4)CC3)[nH]n2)on1] | | |
| 156 | 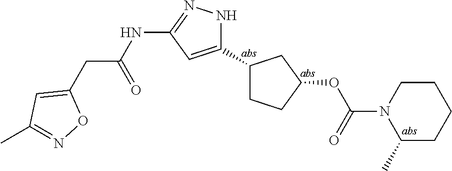 [SMILES: Cc1cc(CC(=O)Nc2cc([C@H]3CC[C@@H](OC(=O)N4CCCC[C@@H]4C)C3)[nH]n2)on1] | | |
| 157 | 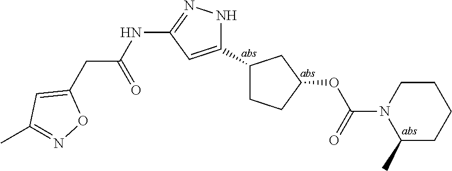 [SMILES: Cc1cc(CC(=O)Nc2cc([C@H]3CC[C@@H](OC(=O)N4CCCC[C@H]4C)C3)[nH]n2)on1] | | |
| 158 | 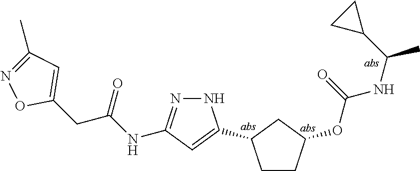 [SMILES: Cc1cc(CC(=O)Nc2cc([C@H]3CC[C@@H](OC(=O)N[C@H](C)C4CC4)C3)[nH]n2)on1] | | |
| 159 | 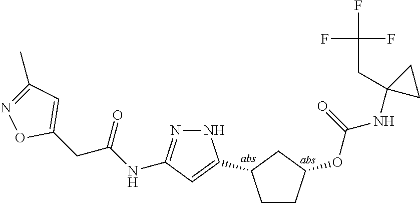 [SMILES: Cc1cc(CC(=O)Nc2cc([C@H]3CC[C@@H](OC(=O)NC4(CC(F)(F)F)CC4)C3)[nH]n2)on1] | | |
| 160 | 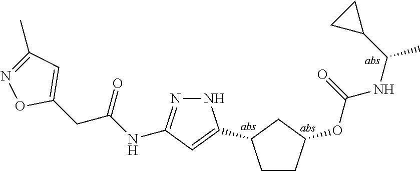 [SMILES: Cc1cc(CC(=O)Nc2cc([C@H]3CC[C@@H](OC(=O)N[C@@H](C)C4CC4)C3)[nH]n2)on1] | | |
| 161 | 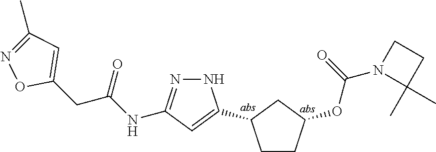 [SMILES: Cc1cc(CC(=O)Nc2cc([C@H]3CC[C@@H](OC(=O)N4CCC4(C)C)C3)[nH]n2)on1] | | |
| 162 | 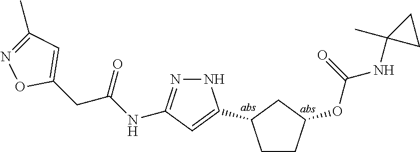 [SMILES: Cc1cc(CC(=O)Nc2cc([C@H]3CC[C@@H](OC(=O)NC4(C)CC4)C3)[nH]n2)on1] | | |
| 163 | 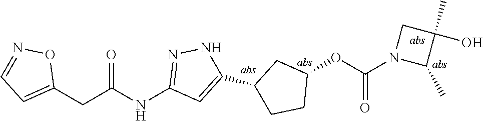 [SMILES: C[C@@H]1N(C(=O)O[C@@H]2CC[C@H](c3cc(NC(=O)Cc4ccno4)n[nH]3)C2)C[C@@]1(C)O] | | |
| 164 | 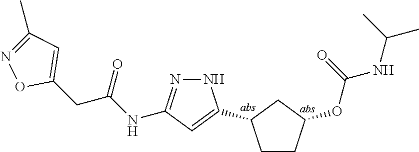 [SMILES: Cc1cc(CC(=O)Nc2cc([C@H]3CC[C@@H](OC(=O)NC(C)C)C3)[nH]n2)on1] | | |
| 165 | 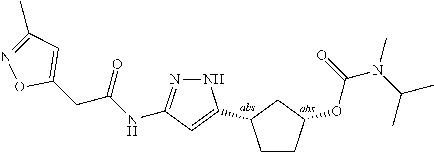 [SMILES: Cc1cc(CC(=O)Nc2cc([C@H]3CC[C@@H](OC(=O)N(C)C(C)C)C3)[nH]n2)on1] | | |
| 166 | 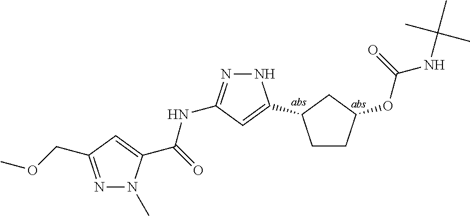 [SMILES: COCc1cc(C(=O)Nc2cc([C@H]3CC[C@@H](OC(=O)NC(C)(C)C)C3)[nH]n2)n(C)n1] | | |
| 167 | 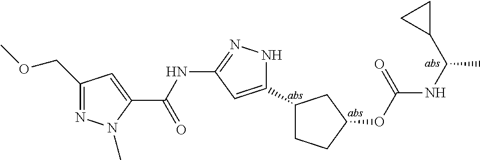 [SMILES: COCc1cc(C(=O)Nc2cc([C@H]3CC[C@@H](OC(=O)N[C@@H](C)C4CC4)C3)[nH]n2)n(C)n1] | | |
| 168 | 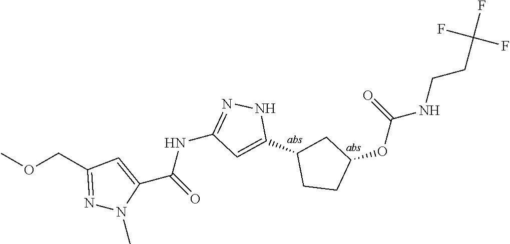 [SMILES: COCc1cc(C(=O)Nc2cc([C@H]3CC[C@@H](OC(=O)NCCC(F)(F)F)C3)[nH]n2)n(C)n1] | | |
| 169 | 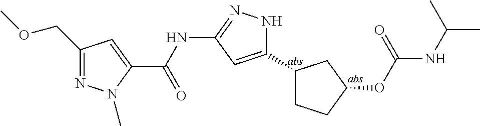 [SMILES: COCc1cc(C(=O)Nc2cc([C@H]3CC[C@@H](OC(=O)NC(C)C)C3)[nH]n2)n(C)n1] | | |
| 170 | 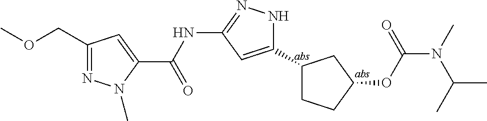 [SMILES: COCc1cc(C(=O)Nc2cc([C@H]3CC[C@@H](OC(=O)N(C)C(C)C)C3)[nH]n2)n(C)n1] | | |
| 171 | 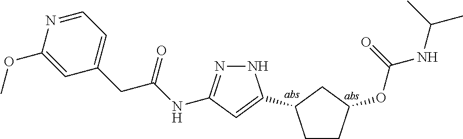 [SMILES: COc1cc(CC(=O)Nc2cc([C@H]3CC[C@@H](OC(=O)NC(C)C)C3)[nH]n2)ccn1] | | |
| 172 | 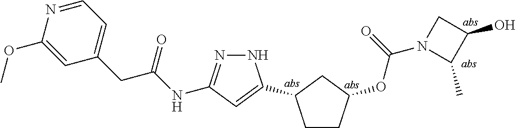 [SMILES: COc1cc(CC(=O)Nc2cc([C@H]3CC[C@@H](OC(=O)N4C[C@@H](O)[C@@H]4C)C3)[nH]n2)ccn1] | | |
| 173 | 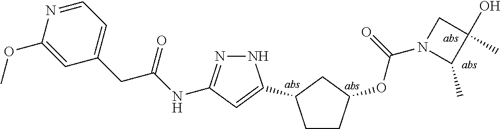 | | |
| 174 | 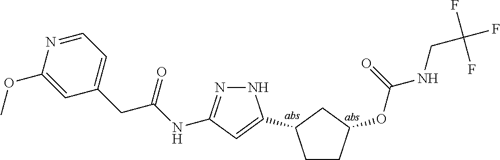 [SMILES: COc1cc(CC(=O)Nc2cc([C@H]3CC[C@@H](OC(=O)NCC(F)(F)F)C3)[nH]n2)ccn1] | | |
| 175 | 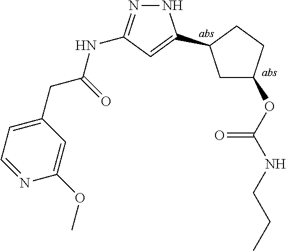 [SMILES: CCCNC(=O)O[C@@H]1CC[C@H](c2cc(NC(=O)Cc3ccnc(OC)c3)n[nH]2)C1] | | |
| 176 | 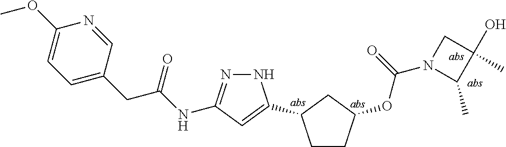 | | |
| 177 | 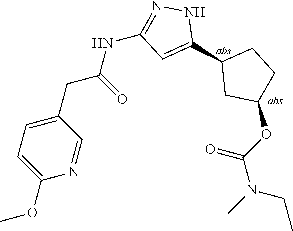 [SMILES: CCN(C)C(=O)O[C@@H]1CC[C@H](c2cc(NC(=O)Cc3ccc(OC)nc3)n[nH]2)C1] | | |
| 178 | 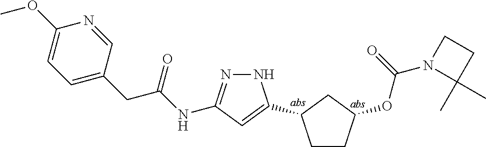 | | |
| 179 | 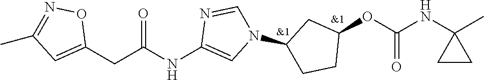 | | |
| 180 | 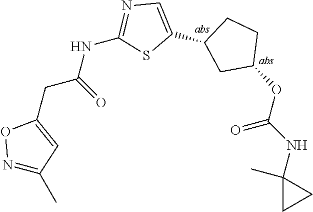 [SMILES: Cc1cc(CC(=O)Nc2ncc([C@@H]3CC[C@H](OC(=O)NC4(C)CC4)C3)s2)on1] | | |
| 181 | 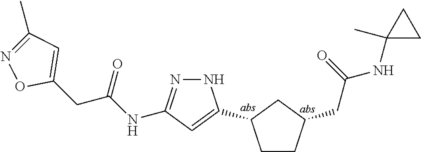 [SMILES: Cc1cc(CC(=O)Nc2cc([C@H]3CC[C@@H](CC(=O)NC4(C)CC4)C3)[nH]n2)on1] | | |
| 182 | 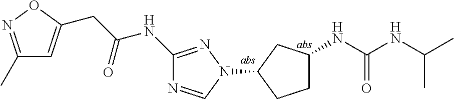 [SMILES: Cc1cc(CC(=O)Nc2ncn([C@H]3CC[C@@H](NC(=O)NC(C)C)C3)n2)on1] | | |
| 183 | 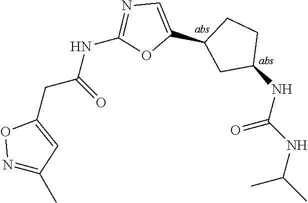 [SMILES: Cc1cc(CC(=O)Nc2ncc([C@H]3CC[C@@H](NC(=O)NC(C)C)C3)o2)on1] | | |
| 184 | 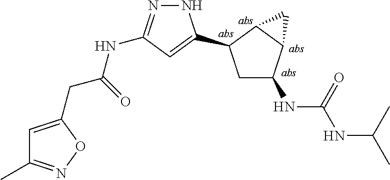 [SMILES: Cc1cc(CC(=O)Nc2cc([C@@H]3C[C@H](NC(=O)NC(C)C)[C@H]4C[C@H]43)[nH]n2)on1] | | |
| 185 | 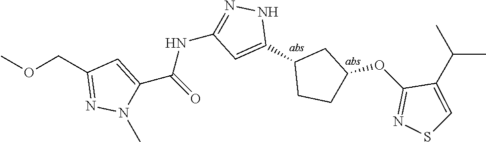 [SMILES: COCc1cc(C(=O)Nc2cc([C@H]3CC[C@@H](Oc4nscc4C(C)C)C3)[nH]n2)n(C)n1] | | |
| 186 | 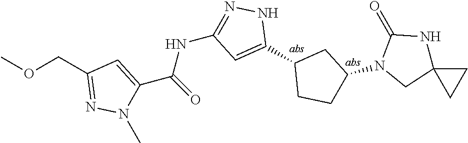 [SMILES: COCc1cc(C(=O)Nc2cc([C@H]3CC[C@@H](N4CC5(CC5)NC4=O)C3)[nH]n2)n(C)n1] | | |
| 187 | 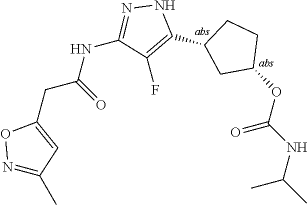 [SMILES: Cc1cc(CC(=O)Nc2n[nH]c([C@@H]3CC[C@H](OC(=O)NC(C)C)C3)c2F)on1] | | |
| 188 | 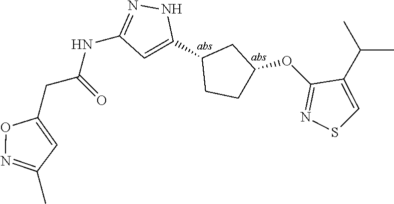 [SMILES: Cc1cc(CC(=O)Nc2cc([C@H]3CC[C@@H](Oc4nscc4C(C)C)C3)[nH]n2)on1] | | |
| 189 | 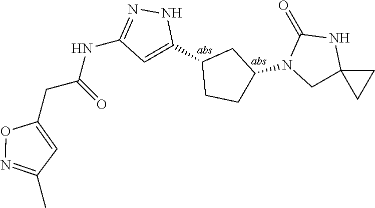 [SMILES: Cc1cc(CC(=O)Nc2cc([C@H]3CC[C@@H](N4CC5(CC5)NC4=O)C3)[nH]n2)on1] | | |
| 190 | 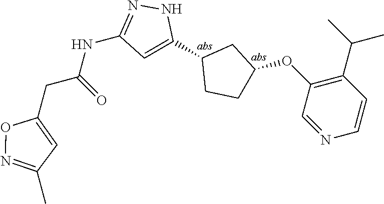 [SMILES: Cc1cc(CC(=O)Nc2cc([C@H]3CC[C@@H](Oc4cnccc4C(C)C)C3)[nH]n2)on1] | | |
| 191 | 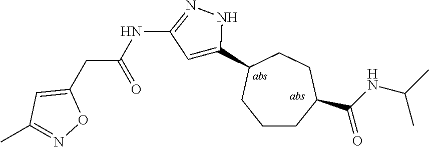 [SMILES: Cc1cc(CC(=O)Nc2cc([C@@H]3CCC[C@H](C(=O)NC(C)C)CC3)[nH]n2)on1] | | |
| 192 | 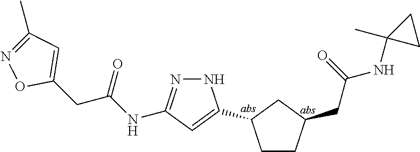 [SMILES: Cc1cc(CC(=O)Nc2cc([C@H]3CC[C@H](CC(=O)NC4(C)CC4)C3)[nH]n2)on1] | | |
| 193 | 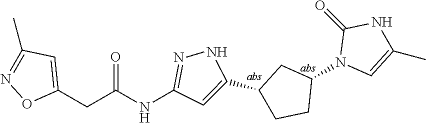 [SMILES: Cc1cc(CC(=O)Nc2cc([C@H]3CC[C@@H](n4cc(C)[nH]c4=O)C3)[nH]n2)on1] | | |
| 194 | 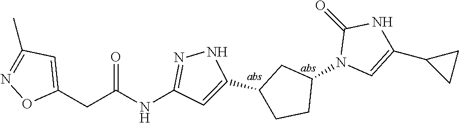 [SMILES: Cc1cc(CC(=O)Nc2cc([C@H]3CC[C@@H](n4cc(C5CC5)[nH]c4=O)C3)[nH]n2)on1] | | |
| 195 | 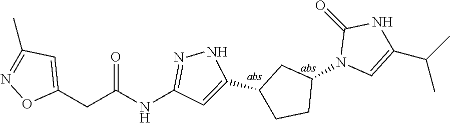 [SMILES: Cc1cc(CC(=O)Nc2cc([C@H]3CC[C@@H](n4cc(C(C)C)[nH]c4=O)C3)[nH]n2)on1] | | |
| 196 | 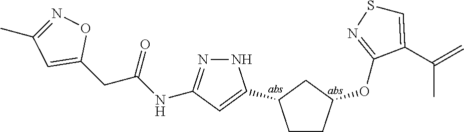 [SMILES: C=C(C)c1csnc1O[C@@H]1CC[C@H](c2cc(NC(=O)Cc3cc(C)no3)n[nH]2)C1] | | |
| 197 | 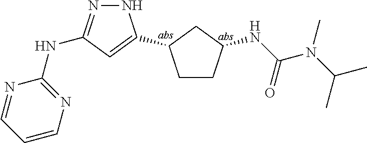 [SMILES: CC(C)N(C)C(=O)N[C@@H]1CC[C@H](c2cc(Nc3ncccn3)n[nH]2)C1] | | |
| 198 | 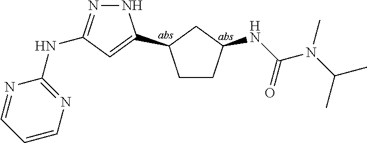 [SMILES: CC(C)N(C)C(=O)N[C@H]1CC[C@@H](c2cc(Nc3ncccn3)n[nH]2)C1] | | |
| 199 | 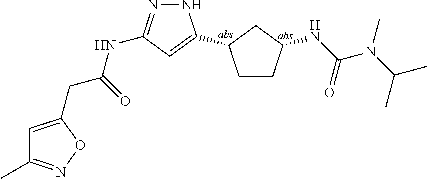 [SMILES: Cc1cc(CC(=O)Nc2cc([C@H]3CC[C@@H](NC(=O)N(C)C(C)C)C3)[nH]n2)on1] | | |
| 200 | 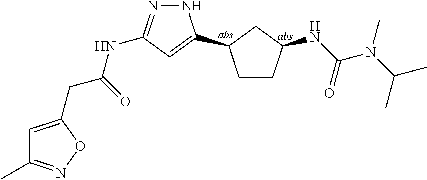 [SMILES: Cc1cc(CC(=O)Nc2cc([C@@H]3CC[C@H](NC(=O)N(C)C(C)C)C3)[nH]n2)on1] | | |
| 201 | 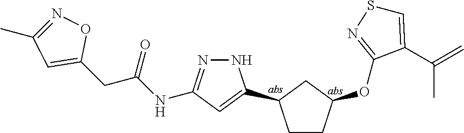 [SMILES: C=C(C)c1csnc1O[C@H]1CC[C@@H](c2cc(NC(=O)Cc3cc(C)no3)n[nH]2)C1] | | |
| 202 | 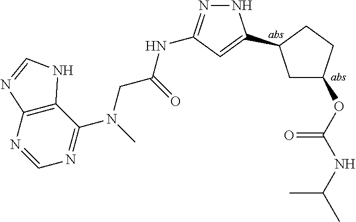 [SMILES: CC(C)NC(=O)O[C@@H]1CC[C@H](c2cc(NC(=O)CN(C)c3ncnc4nc[nH]c34)n[nH]2)C1] | | |
| 203 | 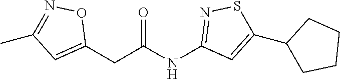 [SMILES: Cc1cc(CC(=O)Nc2cc(C3CCCC3)sn2)on1] | | |
| 204 | 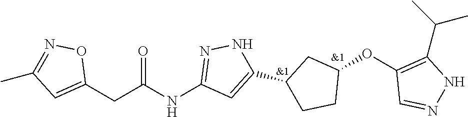 [SMILES: Cc1cc(CC(=O)Nc2cc([C@H]3CC[C@@H](Oc4cn[nH]c4C(C)C)C3)[nH]n2)on1] | | |
| 205 | 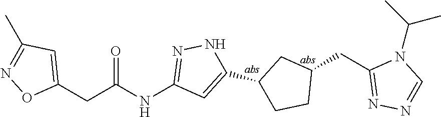 [SMILES: Cc1cc(CC(=O)Nc2cc([C@H]3CC[C@@H](Cc4nncn4C(C)C)C3)[nH]n2)on1] | | |
| 206 | 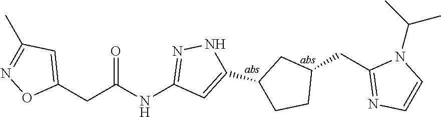 [SMILES: Cc1cc(CC(=O)Nc2cc([C@H]3CC[C@@H](Cc4nccn4C(C)C)C3)[nH]n2)on1] | | |
| 207 | 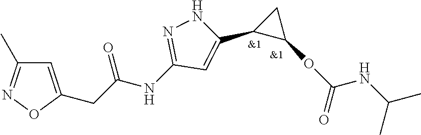 [SMILES: Cc1cc(CC(=O)Nc2cc([C@H]3C[C@H]3OC(=O)NC(C)C)[nH]n2)on1] | | |
| 208 | 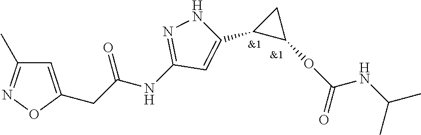 [SMILES: Cc1cc(CC(=O)Nc2cc([C@@H]3C[C@@H]3OC(=O)NC(C)C)[nH]n2)on1] | | |
| 209 | 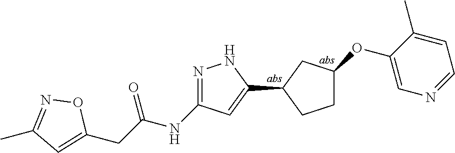 [SMILES: Cc1cc(CC(=O)Nc2cc([C@@H]3CC[C@H](Oc4cnccc4C)C3)[nH]n2)on1] | | |
| 210 | 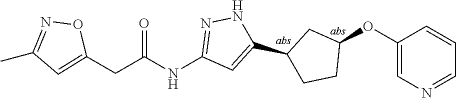 [SMILES: Cc1cc(CC(=O)Nc2cc([C@@H]3CC[C@H](Oc4cccnc4)C3)[nH]n2)on1] | | |
| 211 | 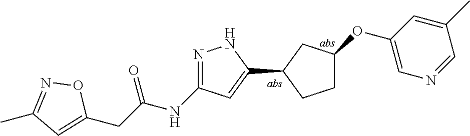 | | |
| 212 | 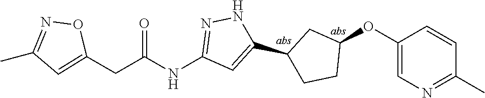 | | |
| 213 | 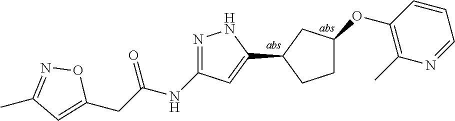 [SMILES: Cc1cc(CC(=O)Nc2cc([C@@H]3CC[C@H](Oc4cccnc4C)C3)[nH]n2)on1] | | |
| 214 | 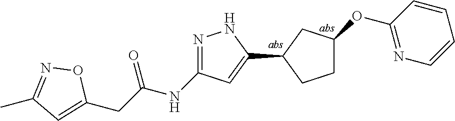 [SMILES: Cc1cc(CC(=O)Nc2cc([C@@H]3CC[C@H](Oc4ccccn4)C3)[nH]n2)on1] | | |
| 215 | 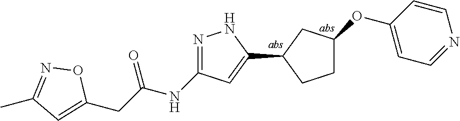 [SMILES: Cc1cc(CC(=O)Nc2cc([C@@H]3CC[C@H](Oc4ccncc4)C3)[nH]n2)on1] | | |
| 216 | 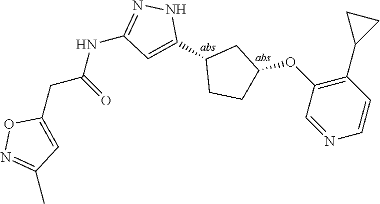 [SMILES: Cc1cc(CC(=O)Nc2cc([C@H]3CC[C@@H](Oc4cnccc4C4CC4)C3)[nH]n2)on1] | | |
| 217 | 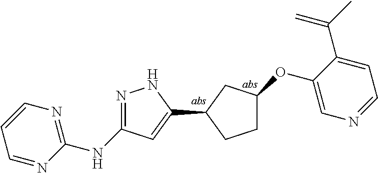 [SMILES: C=C(C)c1ccncc1O[C@H]1CC[C@@H](c2cc(Nc3ncccn3)n[nH]2)C1] | | |
| 218 | 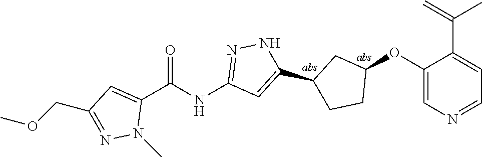 [SMILES: C=C(C)c1ccncc1O[C@H]1CC[C@@H](c2cc(NC(=O)c3cc(COC)nn3C)n[nH]2)C1] | | |
| 219 | 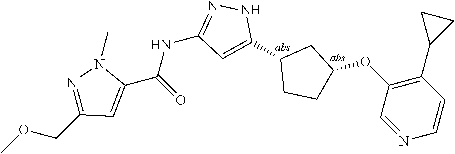 [SMILES: COCc1cc(C(=O)Nc2cc([C@H]3CC[C@@H](Oc4cnccc4C4CC4)C3)[nH]n2)n(C)n1] | | |
| 220 | 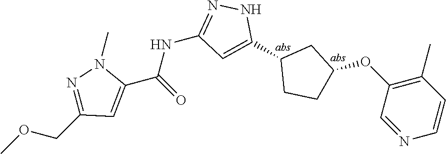 [SMILES: COCc1cc(C(=O)Nc2cc([C@H]3CC[C@@H](Oc4cnccc4C)C3)[nH]n2)n(C)n1] | | |
| 221 | 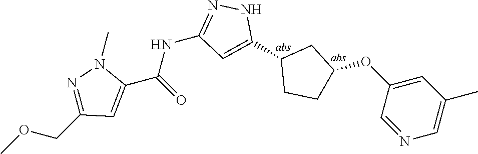 | | |
| 222 | 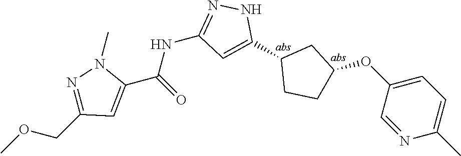 | | |
| 223 | 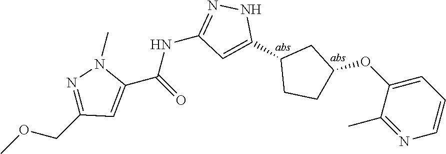 [SMILES: COCc1cc(C(=O)Nc2cc([C@H]3CC[C@@H](Oc4cccnc4C)C3)[nH]n2)n(C)n1] | | |
| 224 | 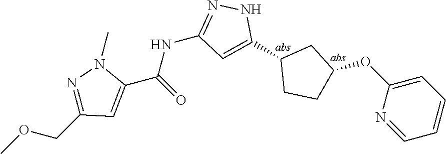 [SMILES: COCc1cc(C(=O)Nc2cc([C@H]3CC[C@@H](Oc4ccccn4)C3)[nH]n2)n(C)n1] | | |
| 225 | 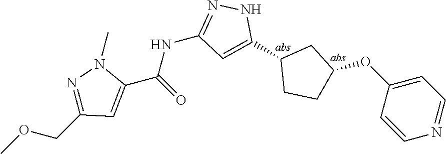 [SMILES: COCc1cc(C(=O)Nc2cc([C@H]3CC[C@@H](Oc4ccncc4)C3)[nH]n2)n(C)n1] | | |
| 226 | 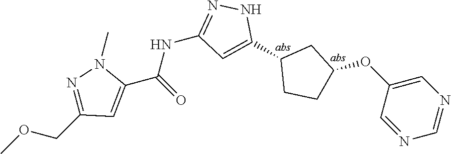 [SMILES: COCc1cc(C(=O)Nc2cc([C@H]3CC[C@@H](Oc4cncnc4)C3)[nH]n2)n(C)n1] | | |
| 227 | 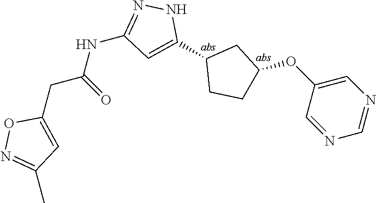 [SMILES: Cc1cc(CC(=O)Nc2cc([C@H]3CC[C@@H](Oc4cncnc4)C3)[nH]n2)on1] | | |
| 228 | 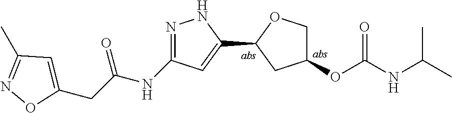 [SMILES: Cc1cc(CC(=O)Nc2cc([C@@H]3C[C@H](OC(=O)NC(C)C)CO3)[nH]n2)on1] | | |
| 229 | 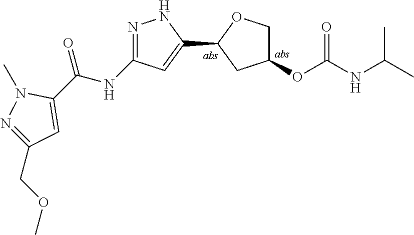 [SMILES: COCc1cc(C(=O)Nc2cc([C@@H]3C[C@H](OC(=O)NC(C)C)CO3)[nH]n2)n(C)n1] | | |
| 230 | 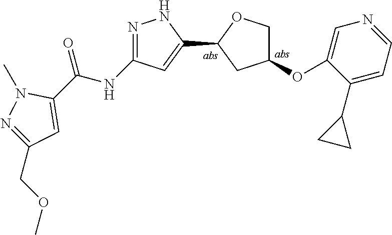 [SMILES: COCc1cc(C(=O)Nc2cc([C@@H]3C[C@H](Oc4cnccc4C4CC4)CO3)[nH]n2)n(C)n1] | | |
| 231 | 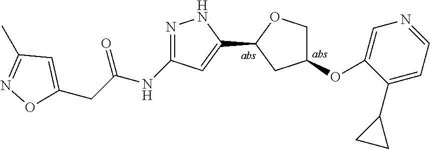 [SMILES: Cc1cc(CC(=O)Nc2cc([C@@H]3C[C@H](Oc4cnccc4C4CC4)CO3)[nH]n2)on1] | | |
| 232 | 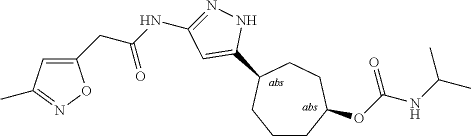 [SMILES: Cc1cc(CC(=O)Nc2cc([C@@H]3CCC[C@H](OC(=O)NC(C)C)CC3)[nH]n2)on1] | | |
| 233 | 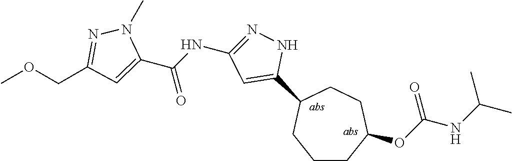 [SMILES: COCc1cc(C(=O)Nc2cc([C@@H]3CCC[C@H](OC(=O)NC(C)C)CC3)[nH]n2)n(C)n1] | | |
| 234 | 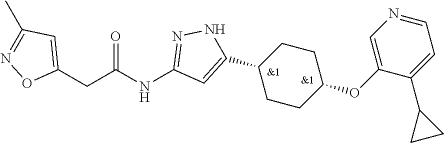 [SMILES: Cc1cc(CC(=O)Nc2cc([C@H]3CC[C@@H](Oc4cnccc4C4CC4)CC3)[nH]n2)on1] | | |
| 235 | 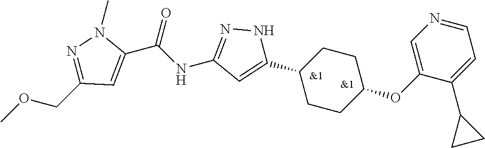 [SMILES: COCc1cc(C(=O)Nc2cc([C@H]3CC[C@@H](Oc4cnccc4C4CC4)CC3)[nH]n2)n(C)n1] | | |
| 236 | 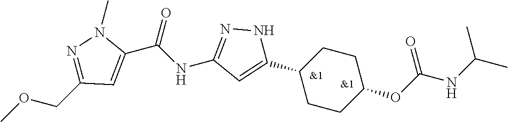 [SMILES: COCc1cc(C(=O)Nc2cc([C@H]3CC[C@@H](OC(=O)NC(C)C)CC3)[nH]n2)n(C)n1] | | |
| 237 | 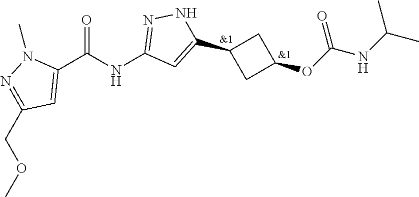 [SMILES: COCc1cc(C(=O)Nc2cc([C@H]3C[C@@H](OC(=O)NC(C)C)C3)[nH]n2)n(C)n1] | | |
| 238 | 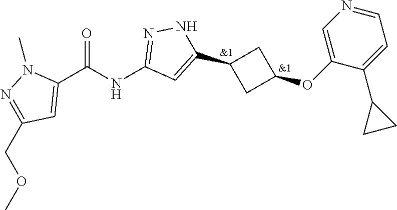 [SMILES: COCc1cc(C(=O)Nc2cc([C@H]3C[C@@H](Oc4cnccc4C4CC4)C3)[nH]n2)n(C)n1] | | |
| 239 | 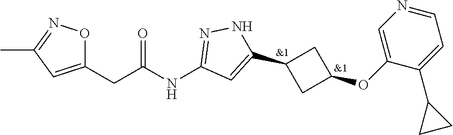 [SMILES: Cc1cc(CC(=O)Nc2cc([C@H]3C[C@@H](Oc4cnccc4C4CC4)C3)[nH]n2)on1] | | |
| 240 | 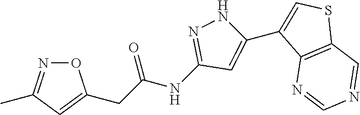 [SMILES: Cc1cc(CC(=O)Nc2cc(-c3csc4cncnc34)[nH]n2)on1] | D | |
| 241 | 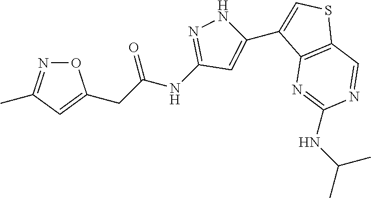 [SMILES: Cc1cc(CC(=O)Nc2cc(-c3csc4cnc(NC(C)C)nc34)[nH]n2)on1] | | |
| 242 | 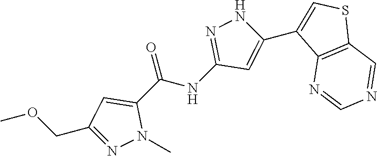 [SMILES: COCc1cc(C(=O)Nc2cc(-c3csc4cncnc34)[nH]n2)n(C)n1] | D | |
| 243 | 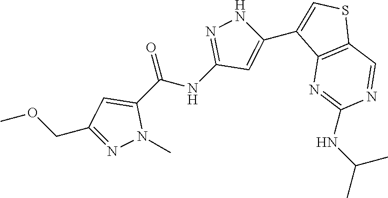 [SMILES: COCc1cc(C(=O)Nc2cc(-c3csc4cnc(NC(C)C)nc34)[nH]n2)n(C)n1] | | |
| 244 | 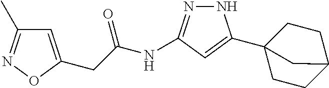 [SMILES: Cc1cc(CC(=O)Nc2cc(C34CCC(CC3)C4)[nH]n2)on1] | D | |
| 245 | 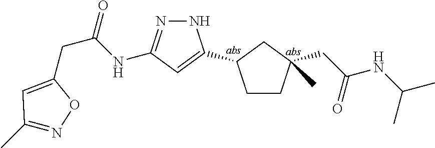 [SMILES: Cc1cc(CC(=O)Nc2cc([C@H]3CC[C@@](C)(CC(=O)NC(C)C)C3)[nH]n2)on1] | | |
| 246 | 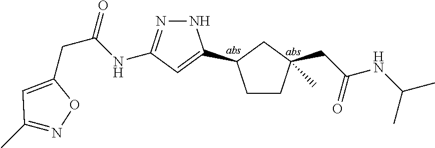 [SMILES: Cc1cc(CC(=O)Nc2cc([C@@H]3CC[C@](C)(CC(=O)NC(C)C)C3)[nH]n2)on1] | | |
| 247 | 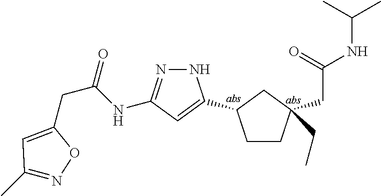 [SMILES: CC[C@@]1(CC(=O)NC(C)C)CC[C@H](c2cc(NC(=O)Cc3cc(C)no3)n[nH]2)C1] | | |
| 248 | 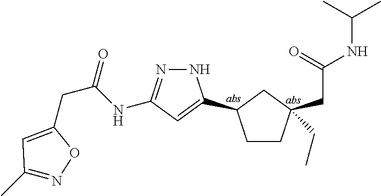 [SMILES: CC[C@]1(CC(=O)NC(C)C)CC[C@@H](c2cc(NC(=O)Cc3cc(C)no3)n[nH]2)C1] | | |
| 249 | 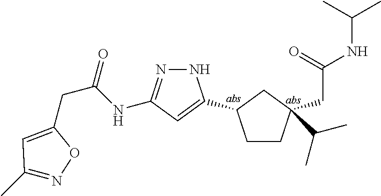 [SMILES: Cc1cc(CC(=O)Nc2cc([C@H]3CC[C@](CC(=O)NC(C)C)(C(C)C)C3)[nH]n2)on1] | | |
| 250 | 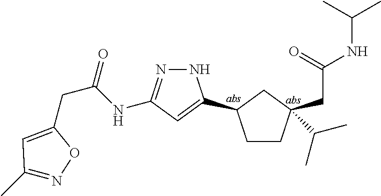 [SMILES: Cc1cc(CC(=O)Nc2cc([C@@H]3CC[C@@](CC(=O)NC(C)C)(C(C)C)C3)[nH]n2)on1] | | |
| 251 | 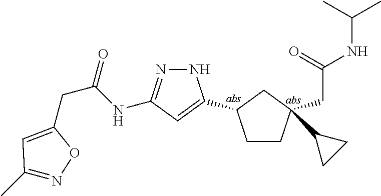 [SMILES: Cc1cc(CC(=O)Nc2cc([C@H]3CC[C@](CC(=O)NC(C)C)(C4CC4)C3)[nH]n2)on1] | | |
| 252 | 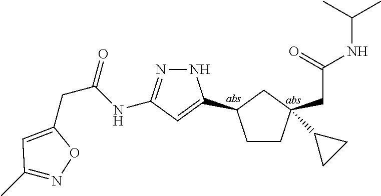 [SMILES: Cc1cc(CC(=O)Nc2cc([C@@H]3CC[C@@](CC(=O)NC(C)C)(C4CC4)C3)[nH]n2)on1] | | |
| 253 | 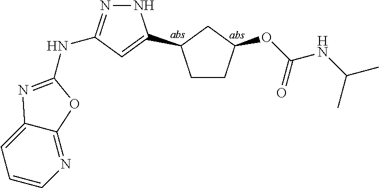 [SMILES: CC(C)NC(=O)O[C@H]1CC[C@@H](c2cc(Nc3nc4cccnc4o3)n[nH]2)C1] | | |
| 254 | 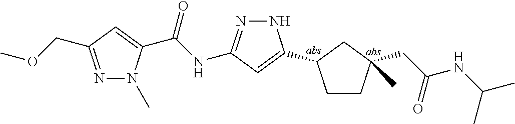 [SMILES: COCc1cc(C(=O)Nc2cc([C@H]3CC[C@@](C)(CC(=O)NC(C)C)C3)[nH]n2)n(C)n1] | | |
| 255 | 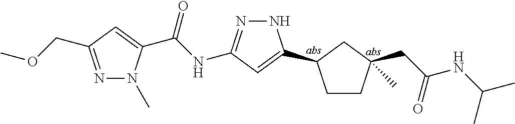 [SMILES: COCc1cc(C(=O)Nc2cc([C@@H]3CC[C@](C)(CC(=O)NC(C)C)C3)[nH]n2)n(C)n1] | | |
| 256 | 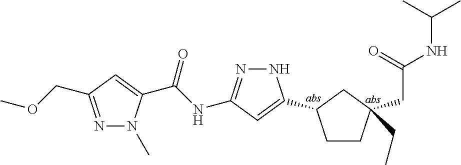 [SMILES: CC[C@@]1(CC(=O)NC(C)C)CC[C@H](c2cc(NC(=O)c3cc(COC)nn3C)n[nH]2)C1] | | |
| 257 | 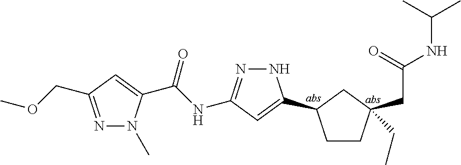 [SMILES: CC[C@]1(CC(=O)NC(C)C)CC[C@@H](c2cc(NC(=O)c3cc(COC)nn3C)n[nH]2)C1] | | |
| 258 | 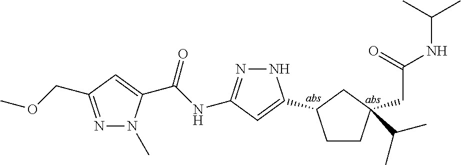 [SMILES: COCc1cc(C(=O)Nc2cc([C@H]3CC[C@](CC(=O)NC(C)C)(C(C)C)C3)[nH]n2)n(C)n1] | | |
| 259 | 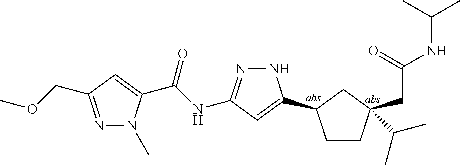 [SMILES: COCc1cc(C(=O)Nc2cc([C@@H]3CC[C@@](CC(=O)NC(C)C)(C(C)C)C3)[nH]n2)n(C)n1] | | |
| 260 | 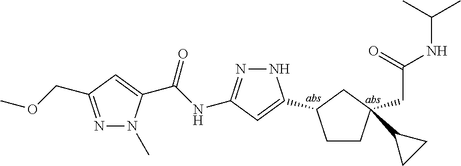 [SMILES: COCc1cc(C(=O)Nc2cc([C@H]3CC[C@](CC(=O)NC(C)C)(C4CC4)C3)[nH]n2)n(C)n1] | | |
| 261 | 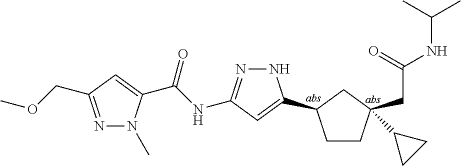 [SMILES: COCc1cc(C(=O)Nc2cc([C@@H]3CC[C@@](CC(=O)NC(C)C)(C4CC4)C3)[nH]n2)n(C)n1] | | |
| 262 | 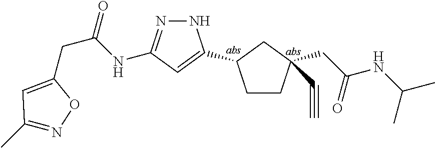 [SMILES: C#C[C@@]1(CC(=O)NC(C)C)CC[C@H](c2cc(NC(=O)Cc3cc(C)no3)n[nH]2)C1] | | |
| 263 | 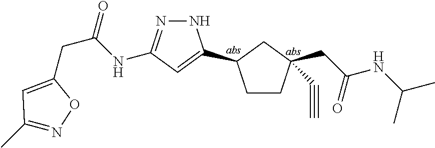 [SMILES: C#C[C@]1(CC(=O)NC(C)C)CC[C@@H](c2cc(NC(=O)Cc3cc(C)no3)n[nH]2)C1] | | |
| 264 | 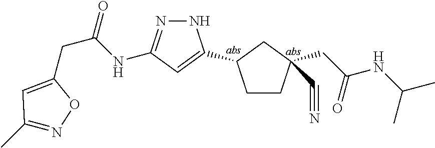 [SMILES: Cc1cc(CC(=O)Nc2cc([C@H]3CC[C@@](C#N)(CC(=O)NC(C)C)C3)[nH]n2)on1] | | |
| 265 | 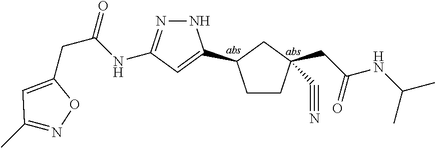 [SMILES: Cc1cc(CC(=O)Nc2cc([C@@H]3CC[C@](C#N)(CC(=O)NC(C)C)C3)[nH]n2)on1] | | |
| 266 | 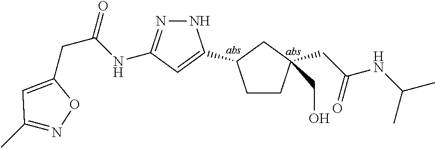 [SMILES: Cc1cc(CC(=O)Nc2cc([C@H]3CC[C@@](CO)(CC(=O)NC(C)C)C3)[nH]n2)on1] | | |
| 267 | 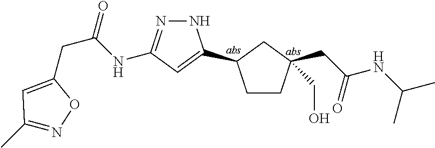 [SMILES: Cc1cc(CC(=O)Nc2cc([C@@H]3CC[C@](CO)(CC(=O)NC(C)C)C3)[nH]n2)on1] | | |
| 268 | 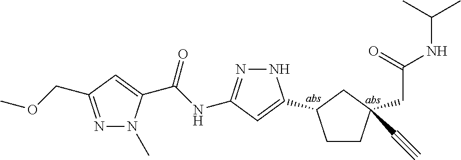 [SMILES: C#C[C@@]1(CC(=O)NC(C)C)CC[C@H](c2cc(NC(=O)c3cc(COC)nn3C)n[nH]2)C1] | | |
| 269 | 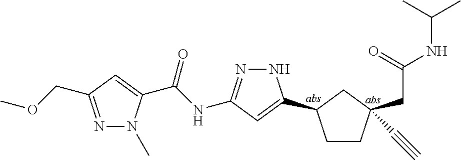 [SMILES: C#C[C@]1(CC(=O)NC(C)C)CC[C@@H](c2cc(NC(=O)c3cc(COC)nn3C)n[nH]2)C1] | | |
| 270 | 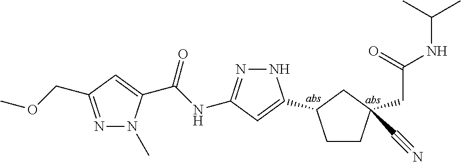 [SMILES: COCc1cc(C(=O)Nc2cc([C@H]3CC[C@@](C#N)(CC(=O)NC(C)C)C3)[nH]n2)n(C)n1] | | |
| 271 | 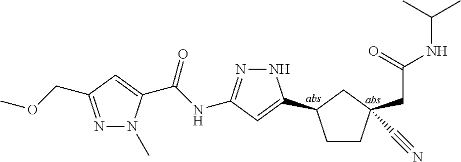 [SMILES: COCc1cc(C(=O)Nc2cc([C@@H]3CC[C@](C#N)(CC(=O)NC(C)C)C3)[nH]n2)n(C)n1] | | |
| 272 | 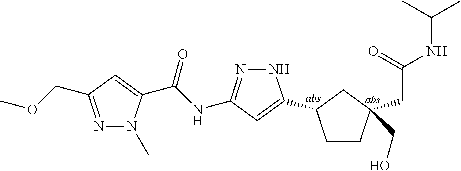 [SMILES: COCc1cc(C(=O)Nc2cc([C@H]3CC[C@@](CO)(CC(=O)NC(C)C)C3)[nH]n2)n(C)n1] | | |
| 273 | 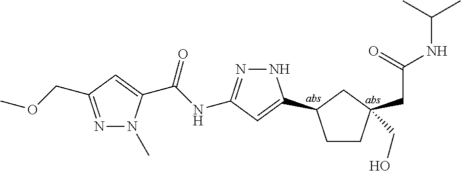 [SMILES: COCc1cc(C(=O)Nc2cc([C@@H]3CC[C@](CO)(CC(=O)NC(C)C)C3)[nH]n2)n(C)n1] | | |
| 274 | 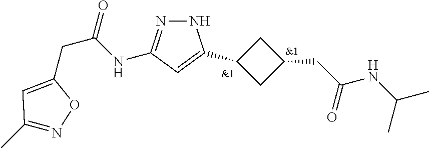 [SMILES: Cc1cc(CC(=O)Nc2cc([C@H]3C[C@@H](CC(=O)NC(C)C)C3)[nH]n2)on1] | | |
| 275 | 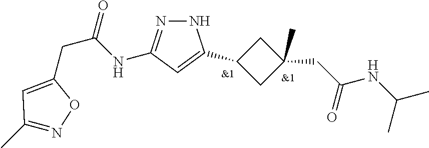 [SMILES: Cc1cc(CC(=O)Nc2cc([C@H]3C[C@@](C)(CC(=O)NC(C)C)C3)[nH]n2)on1] | | |
| 276 | 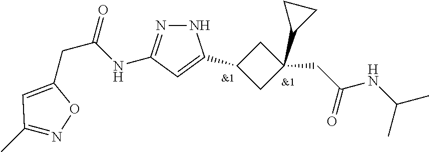 [SMILES: Cc1cc(CC(=O)Nc2cc([C@H]3C[C@](CC(=O)NC(C)C)(C4CC4)C3)[nH]n2)on1] | | |
| 277 | 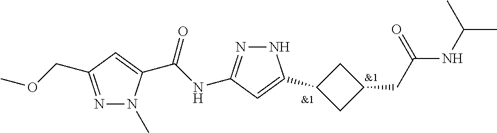 [SMILES: COCc1cc(C(=O)Nc2cc([C@H]3C[C@@H](CC(=O)NC(C)C)C3)[nH]n2)n(C)n1] | | |
| 278 | 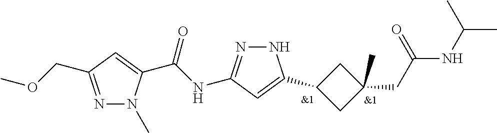 [SMILES: COCc1cc(C(=O)Nc2cc([C@H]3C[C@@](C)(CC(=O)NC(C)C)C3)[nH]n2)n(C)n1] | | |
| 279 | 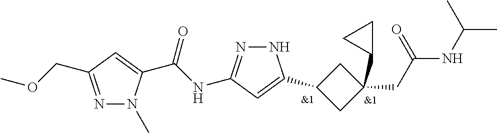 [SMILES: COCc1cc(C(=O)Nc2cc([C@H]3C[C@](CC(=O)NC(C)C)(C4CC4)C3)[nH]n2)n(C)n1] | | |
| 280 | 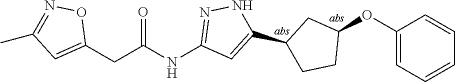 [SMILES: Cc1cc(CC(=O)Nc2cc([C@@H]3CC[C@H](Oc4ccccc4)C3)[nH]n2)on1] | | |
| 281 | 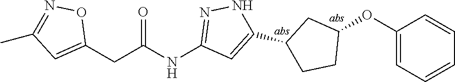 [SMILES: Cc1cc(CC(=O)Nc2cc([C@H]3CC[C@@H](Oc4ccccc4)C3)[nH]n2)on1] | | |
| 282 | 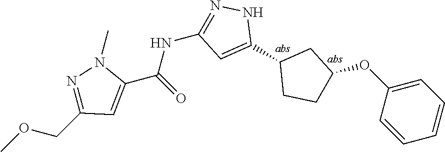 [SMILES: COCc1cc(C(=O)Nc2cc([C@H]3CC[C@@H](Oc4ccccc4)C3)[nH]n2)n(C)n1] | | |
| 283 | 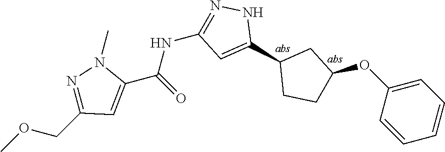 [SMILES: COCc1cc(C(=O)Nc2cc([C@@H]3CC[C@H](Oc4ccccc4)C3)[nH]n2)n(C)n1] | | |
| 284 | 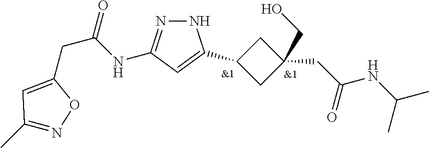 [SMILES: Cc1cc(CC(=O)Nc2cc([C@H]3C[C@@](CO)(CC(=O)NC(C)C)C3)[nH]n2)on1] | | |
| 285 | 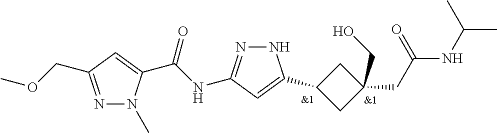 [SMILES: COCc1cc(C(=O)Nc2cc([C@H]3C[C@@](CO)(CC(=O)NC(C)C)C3)[nH]n2)n(C)n1] | | |
| 286 | 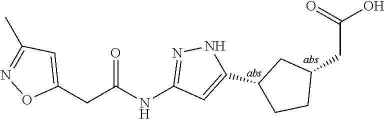 [SMILES: Cc1cc(CC(=O)Nc2cc([C@H]3CC[C@@H](CC(=O)O)C3)[nH]n2)on1] | | |
| 287 | 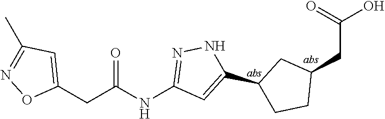 [SMILES: Cc1cc(CC(=O)Nc2cc([C@@H]3CC[C@H](CC(=O)O)C3)[nH]n2)on1] | | |
| 288 | 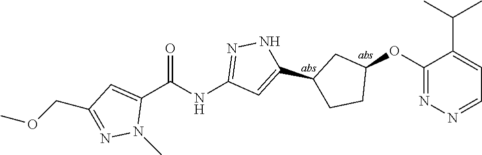 [SMILES: COCc1cc(C(=O)Nc2cc([C@@H]3CC[C@H](Oc4nnccc4C(C)C)C3)[nH]n2)n(C)n1] | | |
| 289 | 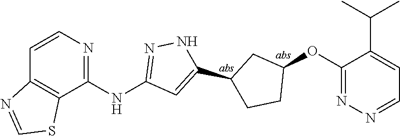 [SMILES: CC(C)c1ccnnc1O[C@H]1CC[C@@H](c2cc(Nc3nccc4ncsc34)n[nH]2)C1] | | |
| 290 | 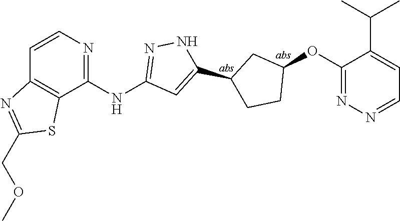 [SMILES: COCc1nc2ccnc(Nc3cc([C@@H]4CC[C@H](Oc5nnccc5C(C)C)C4)[nH]n3)c2s1] | | |
| 291 | 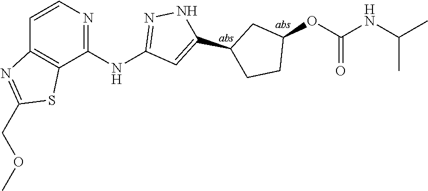 [SMILES: COCc1nc2ccnc(Nc3cc([C@@H]4CC[C@H](OC(=O)NC(C)C)C4)[nH]n3)c2s1] | | |
| 292 | 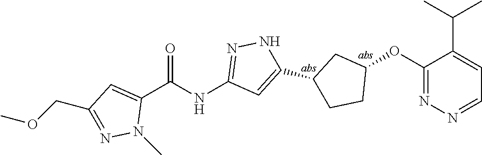 [SMILES: COCc1cc(C(=O)Nc2cc([C@H]3CC[C@@H](Oc4nnccc4C(C)C)C3)[nH]n2)n(C)n1] | | |
| 293 | 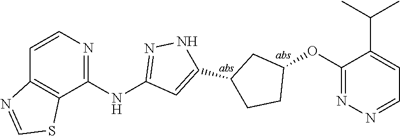 [SMILES: CC(C)c1ccnnc1O[C@@H]1CC[C@H](c2cc(Nc3nccc4ncsc34)n[nH]2)C1] | | |
| 294 | 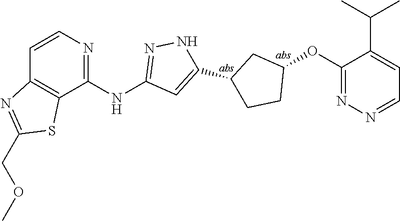 [SMILES: COCc1nc2ccnc(Nc3cc([C@H]4CC[C@@H](Oc5nnccc5C(C)C)C4)[nH]n3)c2s1] | | |
| 295 | 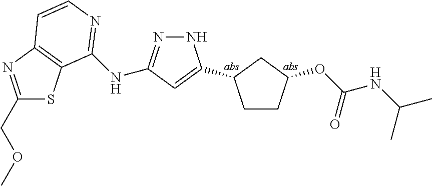 [SMILES: COCc1nc2ccnc(Nc3cc([C@H]4CC[C@@H](OC(=O)NC(C)C)C4)[nH]n3)c2s1] | | |
| 296 | 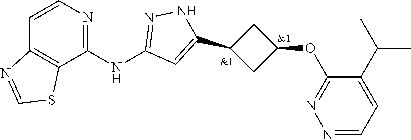 [SMILES: CC(C)c1ccnnc1O[C@H]1C[C@@H](c2cc(Nc3nccc4ncsc34)n[nH]2)C1] | | |
| 297 | 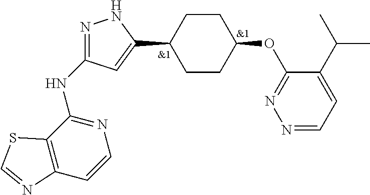 [SMILES: CC(C)c1ccnnc1O[C@H]1CC[C@@H](c2cc(Nc3nccc4ncsc34)n[nH]2)CC1] | | |
| 298 | 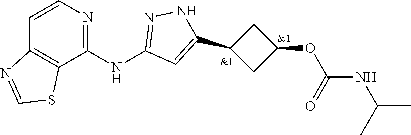 [SMILES: CC(C)NC(=O)O[C@H]1C[C@@H](c2cc(Nc3nccc4ncsc34)n[nH]2)C1] | | |
| 299 | 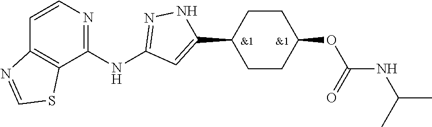 [SMILES: CC(C)NC(=O)O[C@H]1CC[C@@H](c2cc(Nc3nccc4ncsc34)n[nH]2)CC1] | | |
| 300 | 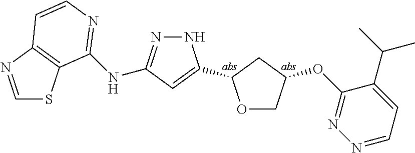 [SMILES: CC(C)c1ccnnc1O[C@@H]1CO[C@H](c2cc(Nc3nccc4ncsc34)n[nH]2)C1] | | |
| 301 | 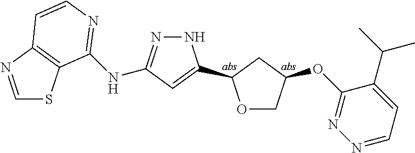 [SMILES: CC(C)c1ccnnc1O[C@H]1CO[C@@H](c2cc(Nc3nccc4ncsc34)n[nH]2)C1] | | |
| 302 | 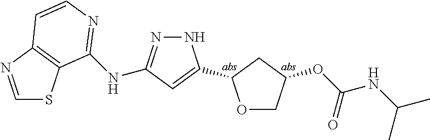 [SMILES: CC(C)NC(=O)O[C@@H]1CO[C@H](c2cc(Nc3nccc4ncsc34)n[nH]2)C1] | | |
| 303 | 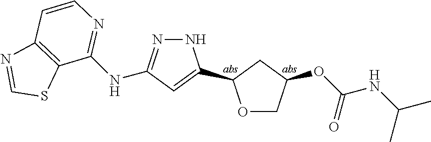 [SMILES: CC(C)NC(=O)O[C@H]1CO[C@@H](c2cc(Nc3nccc4ncsc34)n[nH]2)C1] | | |
| 304 | 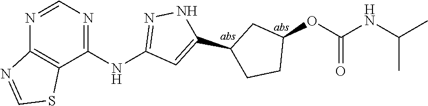 [SMILES: CC(C)NC(=O)O[C@H]1CC[C@@H](c2cc(Nc3ncnc4ncsc34)n[nH]2)C1] | | |
| 305 | 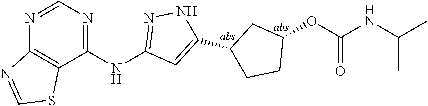 [SMILES: CC(C)NC(=O)O[C@@H]1CC[C@H](c2cc(Nc3ncnc4ncsc34)n[nH]2)C1] | | |
| 306 | 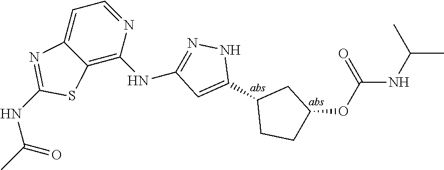 [SMILES: CC(=O)Nc1nc2ccnc(Nc3cc([C@H]4CC[C@@H](OC(=O)NC(C)C)C4)[nH]n3)c2s1] | | |
| 307 | 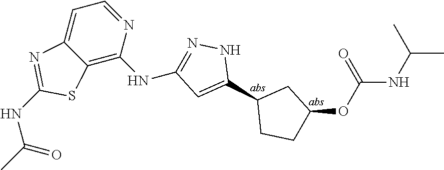 [SMILES: CC(=O)Nc1nc2ccnc(Nc3cc([C@@H]4CC[C@H](OC(=O)NC(C)C)C4)[nH]n3)c2s1] | | |
| 308 | 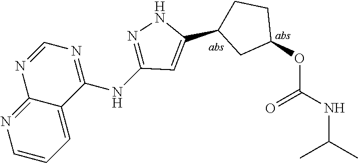 [SMILES: CC(C)NC(=O)O[C@@H]1CC[C@H](c2cc(Nc3ncnc4ncccc34)n[nH]2)C1] | | |
| 309 | 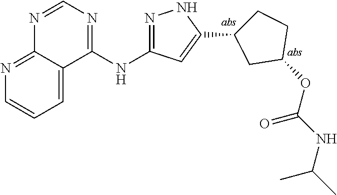 [SMILES: CC(C)NC(=O)O[C@H]1CC[C@@H](c2cc(Nc3ncnc4ncccc34)n[nH]2)C1] | | |
| 310 | 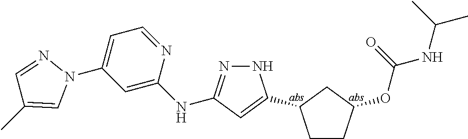 [SMILES: Cc1cnn(-c2ccnc(Nc3cc([C@H]4CC[C@@H](OC(=O)NC(C)C)C4)[nH]n3)c2)c1] | | |
| 311 | 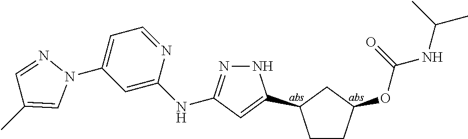 [SMILES: Cc1cnn(-c2ccnc(Nc3cc([C@@H]4CC[C@H](OC(=O)NC(C)C)C4)[nH]n3)c2)c1] | | |
| 312 | 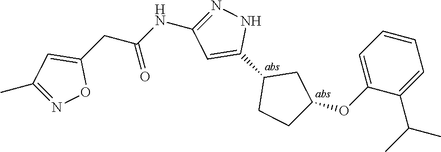 [SMILES: Cc1cc(CC(=O)Nc2cc([C@H]3CC[C@@H](Oc4ccccc4C(C)C)C3)[nH]n2)on1] | | |
| 313 | 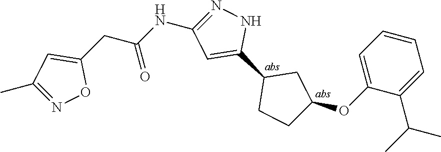 [SMILES: Cc1cc(CC(=O)Nc2cc([C@@H]3CC[C@H](Oc4ccccc4C(C)C)C3)[nH]n2)on1] | | |
| 314 | 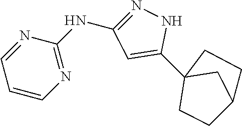 [SMILES: c1cnc(Nc2cc(C34CCC(CC3)C4)[nH]n2)nc1] | D | |
| 315 | 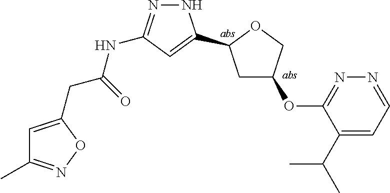 [SMILES: Cc1cc(CC(=O)Nc2cc([C@@H]3C[C@H](Oc4nnccc4C(C)C)CO3)[nH]n2)on1] | | |
| 316 | 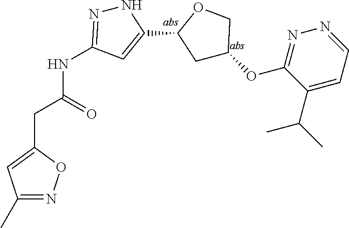 [SMILES: Cc1cc(CC(=O)Nc2cc([C@H]3C[C@@H](Oc4nnccc4C(C)C)CO3)[nH]n2)on1] | | |
| 317 | 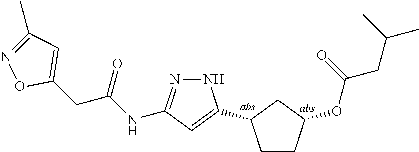 [SMILES: Cc1cc(CC(=O)Nc2cc([C@H]3CC[C@@H](OC(=O)CC(C)C)C3)[nH]n2)on1] | | |
| 318 | 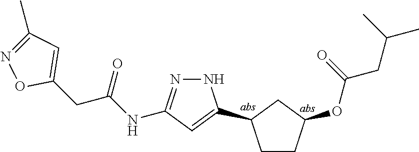 [SMILES: Cc1cc(CC(=O)Nc2cc([C@@H]3CC[C@H](OC(=O)CC(C)C)C3)[nH]n2)on1] | | |
| 319 | 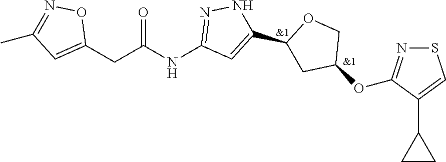 [SMILES: Cc1cc(CC(=O)Nc2cc([C@@H]3C[C@H](Oc4nscc4C4CC4)CO3)[nH]n2)on1] | | |
| 320 | 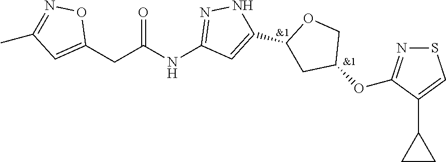 [SMILES: Cc1cc(CC(=O)Nc2cc([C@H]3C[C@@H](Oc4nscc4C4CC4)CO3)[nH]n2)on1] | | |
| 321 | 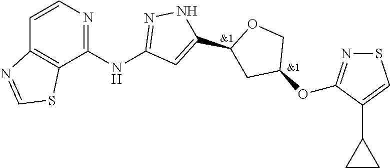 [SMILES: c1cc2ncsc2c(Nc2cc([C@@H]3C[C@H](Oc4nscc4C4CC4)CO3)[nH]n2)n1] | | |
| 322 | 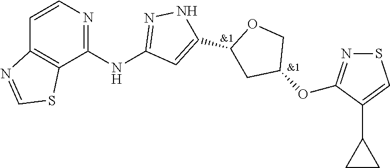 [SMILES: c1cc2ncsc2c(Nc2cc([C@H]3C[C@@H](Oc4nscc4C4CC4)CO3)[nH]n2)n1] | | |
| 323 | 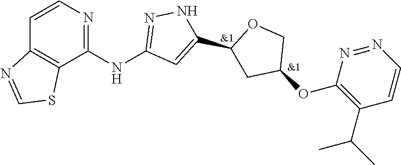 [SMILES: CC(C)c1ccnnc1O[C@@H]1CO[C@H](c2cc(Nc3nccc4ncsc34)n[nH]2)C1] | | |
| 324 | 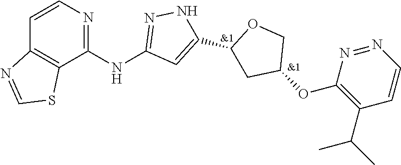 [SMILES: CC(C)c1ccnnc1O[C@H]1CO[C@@H](c2cc(Nc3nccc4ncsc34)n[nH]2)C1] | | |
| 325 | 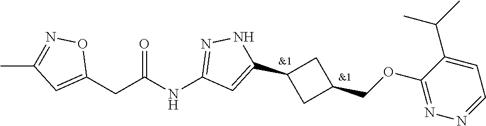 [SMILES: Cc1cc(CC(=O)Nc2cc([C@H]3C[C@@H](COc4nnccc4C(C)C)C3)[nH]n2)on1] | | |
| 326 | 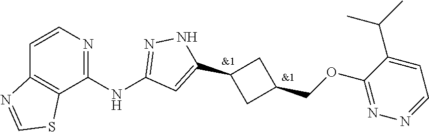 [SMILES: CC(C)c1ccnnc1OC[C@H]1C[C@@H](c2cc(Nc3nccc4ncsc34)n[nH]2)C1] | | |
| 327 | 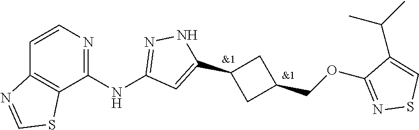 [SMILES: CC(C)c1csnc1OC[C@H]1C[C@@H](c2cc(Nc3nccc4ncsc34)n[nH]2)C1] | | |
| 328 | 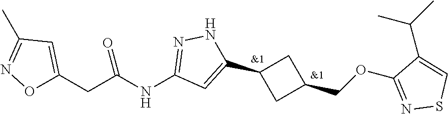 [SMILES: Cc1cc(CC(=O)Nc2cc([C@H]3C[C@@H](COc4nscc4C(C)C)C3)[nH]n2)on1] | | |
| 329 | 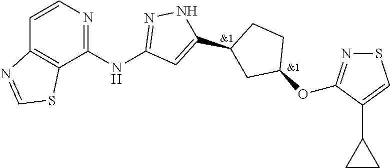 [SMILES: c1cc2ncsc2c(Nc2cc([C@H]3CC[C@@H](Oc4nscc4C4CC4)C3)[nH]n2)n1] | | |
| 330 | 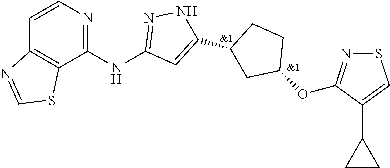 [SMILES: c1cc2ncsc2c(Nc2cc([C@@H]3CC[C@H](Oc4nscc4C4CC4)C3)[nH]n2)n1] | | |
| 331 | 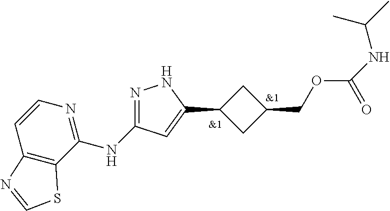 [SMILES: CC(C)NC(=O)OC[C@H]1C[C@@H](c2cc(Nc3nccc4ncsc34)n[nH]2)C1] | | |
| 332 | 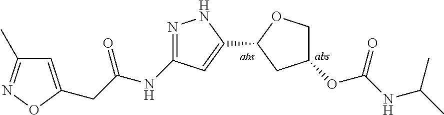 [SMILES: Cc1cc(CC(=O)Nc2cc([C@H]3C[C@@H](OC(=O)NC(C)C)CO3)[nH]n2)on1] | | |
| 333 | 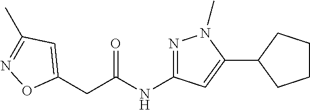 [SMILES: Cc1cc(CC(=O)Nc2cc(C3CCCC3)n(C)n2)on1] | | |
| 334 | 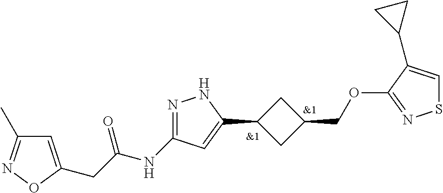 [SMILES: Cc1cc(CC(=O)Nc2cc([C@H]3C[C@@H](COc4nscc4C4CC4)C3)[nH]n2)on1] | | |
| 335 | 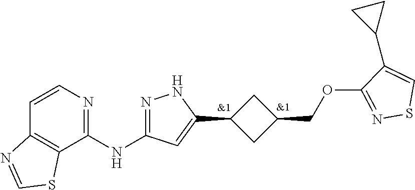 [SMILES: c1cc2ncsc2c(Nc2cc([C@H]3C[C@@H](COc4nscc4C4CC4)C3)[nH]n2)n1] | | |
| 336 | 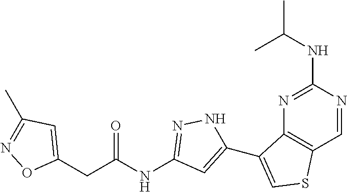 [SMILES: Cc1cc(CC(=O)Nc2cc(-c3csc4cnc(NC(C)C)nc34)[nH]n2)on1] | C | |
| 337 | 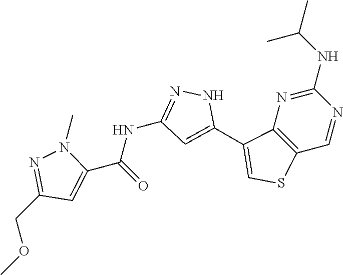 [SMILES: COCc1cc(C(=O)Nc2cc(-c3csc4cnc(NC(C)C)nc34)[nH]n2)n(C)n1] | D | |
| 338 | 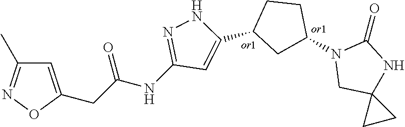 [SMILES: Cc1cc(CC(=O)Nc2cc([C@@H]3CC[C@H](N4CC5(CC5)NC4=O)C3)[nH]n2)on1] | C | |
| 339 | 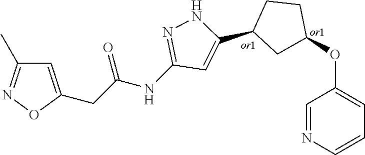 [SMILES: Cc1cc(CC(=O)Nc2cc([C@H]3CC[C@@H](Oc4cccnc4)C3)[nH]n2)on1] | C | |
| 340 | 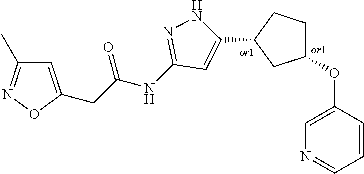 [SMILES: Cc1cc(CC(=O)Nc2cc([C@@H]3CC[C@H](Oc4cccnc4)C3)[nH]n2)on1] | D | |
| 341 | 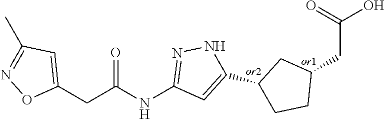 [SMILES: Cc1cc(CC(=O)Nc2cc([C@H]3CC[C@@H](CC(=O)O)C3)[nH]n2)on1] | D | |
| 342 | 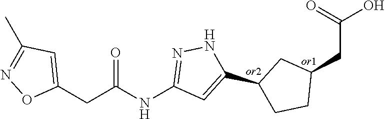 [SMILES: Cc1cc(CC(=O)Nc2cc([C@@H]3CC[C@H](CC(=O)O)C3)[nH]n2)on1] | D | |
| 343 | 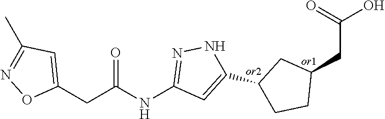 [SMILES: Cc1cc(CC(=O)Nc2cc([C@H]3CC[C@H](CC(=O)O)C3)[nH]n2)on1] | D | |
| 344 | 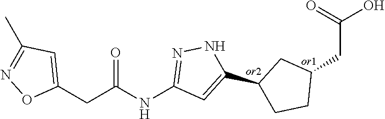 [SMILES: Cc1cc(CC(=O)Nc2cc([C@@H]3CC[C@@H](CC(=O)O)C3)[nH]n2)on1] | D | |
| 345 | 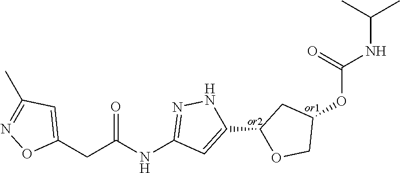 [SMILES: Cc1cc(CC(=O)Nc2cc([C@@H]3C[C@H](OC(=O)NC(C)C)CO3)[nH]n2)on1] | B | |
| 346 | 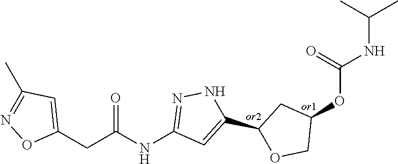 [SMILES: Cc1cc(CC(=O)Nc2cc([C@H]3C[C@@H](OC(=O)NC(C)C)CO3)[nH]n2)on1] | B | |
| 347 | 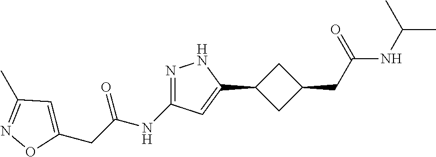 [SMILES: Cc1cc(CC(=O)Nc2cc([C@H]3C[C@@H](CC(=O)NC(C)C)C3)[nH]n2)on1] | D | |
| 348 | 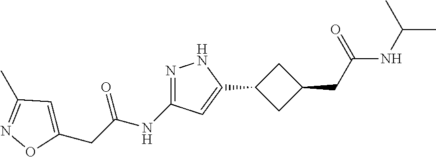 [SMILES: Cc1cc(CC(=O)Nc2cc([C@H]3C[C@H](CC(=O)NC(C)C)C3)[nH]n2)on1] | D | |
| 349 | 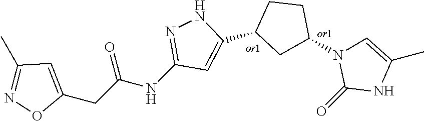 | C | |
| 350 | 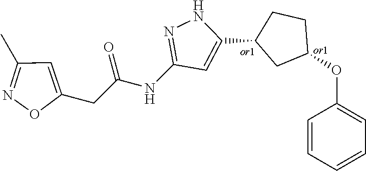 [SMILES: Cc1cc(CC(=O)Nc2cc([C@@H]3CC[C@H](Oc4ccccc4)C3)[nH]n2)on1] | D | |
| 351 | 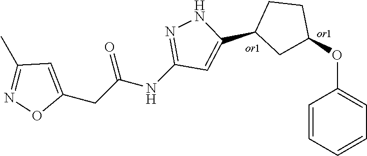 [SMILES: Cc1cc(CC(=O)Nc2cc([C@H]3CC[C@@H](Oc4ccccc4)C3)[nH]n2)on1] | B | |
| 352 | 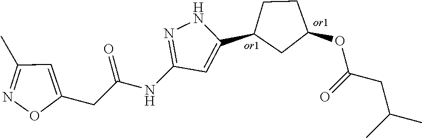 [SMILES: Cc1cc(CC(=O)Nc2cc([C@H]3CC[C@@H](OC(=O)CC(C)C)C3)[nH]n2)on1] | A | |
| 353 | 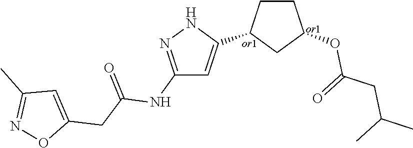 [SMILES: Cc1cc(CC(=O)Nc2cc([C@@H]3CC[C@H](OC(=O)CC(C)C)C3)[nH]n2)on1] | A | |
| 354 | 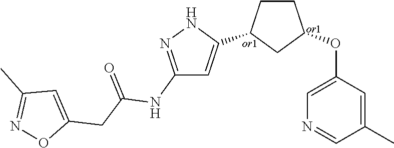 | D | |
| 355 | 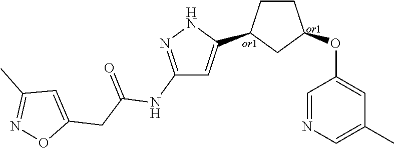 | C | |
| 356 | 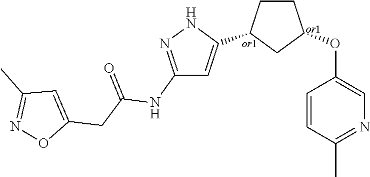 [SMILES: Cc1ccc(O[C@H]2CC[C@@H](c3cc(NC(=O)Cc4cc(C)no4)n[nH]3)C2)cn1] | C | |
| 357 | 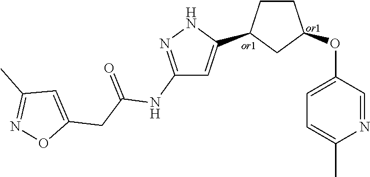 [SMILES: Cc1ccc(O[C@@H]2CC[C@H](c3cc(NC(=O)Cc4cc(C)no4)n[nH]3)C2)cn1] | D | |
| 358 | 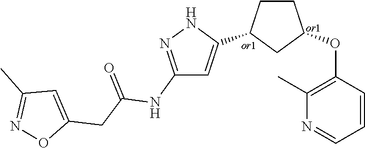 [SMILES: Cc1cc(CC(=O)Nc2cc([C@@H]3CC[C@H](Oc4cccnc4C)C3)[nH]n2)on1] | C | |
| 359 | 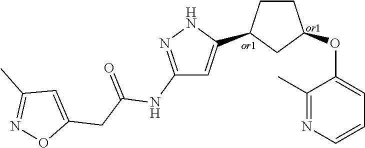 [SMILES: Cc1cc(CC(=O)Nc2cc([C@H]3CC[C@@H](Oc4cccnc4C)C3)[nH]n2)on1] | B | |
| 360 | 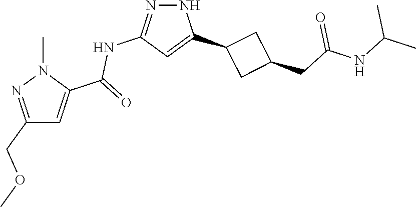 [SMILES: COCc1cc(C(=O)Nc2cc([C@H]3C[C@@H](CC(=O)NC(C)C)C3)[nH]n2)n(C)n1] | D | |
| 361 | 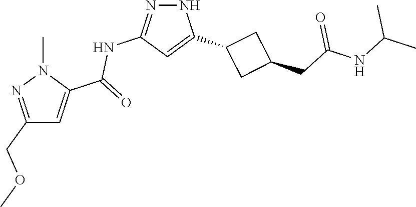 [SMILES: COCc1cc(C(=O)Nc2cc([C@H]3C[C@H](CC(=O)NC(C)C)C3)[nH]n2)n(C)n1] | D | |
| 362 | 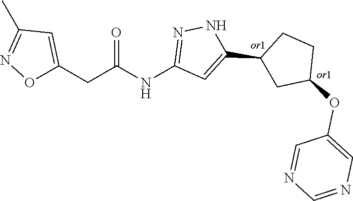 [SMILES: Cc1cc(CC(=O)Nc2cc([C@H]3CC[C@@H](Oc4cncnc4)C3)[nH]n2)on1] | D | |
| 363 | 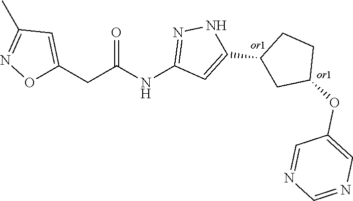 [SMILES: Cc1cc(CC(=O)Nc2cc([C@@H]3CC[C@H](Oc4cncnc4)C3)[nH]n2)on1] | C | |
| 364 | 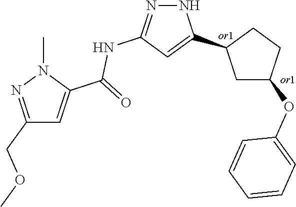 [SMILES: COCc1cc(C(=O)Nc2cc([C@H]3CC[C@@H](Oc4ccccc4)C3)[nH]n2)n(C)n1] | D | |
| 365 | 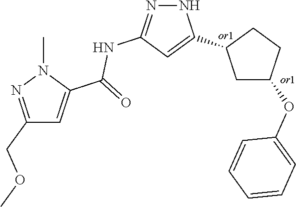 [SMILES: COCc1cc(C(=O)Nc2cc([C@@H]3CC[C@H](Oc4ccccc4)C3)[nH]n2)n(C)n1] | C | |
| 366 | 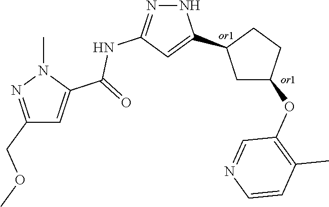 | D | |
| 367 | 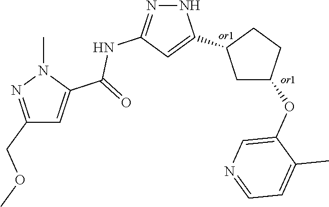 | B | |
| 368 | 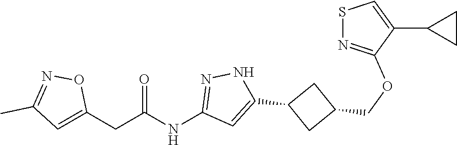 [SMILES: Cc1cc(CC(=O)Nc2cc([C@H]3C[C@@H](COc4nscc4C4CC4)C3)[nH]n2)on1] | A | |
| 369 | 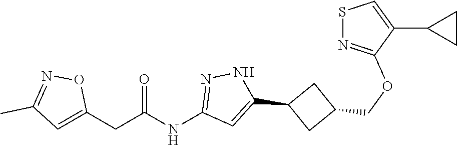 [SMILES: Cc1cc(CC(=O)Nc2cc([C@H]3C[C@H](COc4nscc4C4CC4)C3)[nH]n2)on1] | D | |
| 370 | 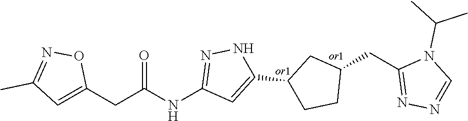 [SMILES: Cc1cc(CC(=O)Nc2cc([C@H]3CC[C@@H](Cc4nncn4C(C)C)C3)[nH]n2)on1] | D | |
| 371 | 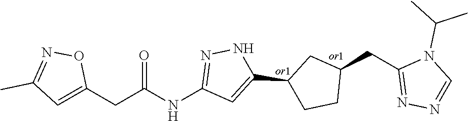 [SMILES: Cc1cc(CC(=O)Nc2cc([C@@H]3CC[C@H](Cc4nncn4C(C)C)C3)[nH]n2)on1] | D | |
| 372 | 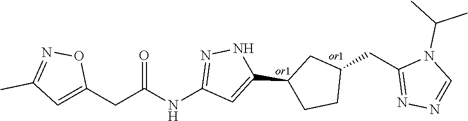 [SMILES: Cc1cc(CC(=O)Nc2cc([C@@H]3CC[C@@H](Cc4nncn4C(C)C)C3)[nH]n2)on1] | D | |
| 373 | 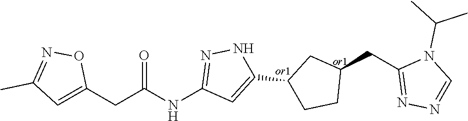 [SMILES: Cc1cc(CC(=O)Nc2cc([C@H]3CC[C@H](Cc4nncn4C(C)C)C3)[nH]n2)on1] | B | |
| 374 | 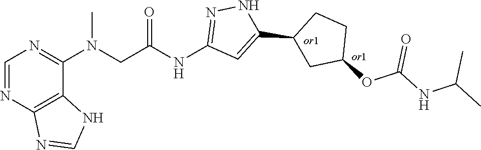 [SMILES: CC(C)NC(=O)O[C@@H]1CC[C@H](c2cc(NC(=O)CN(C)c3ncnc4nc[nH]c34)n[nH]2)C1] | B | |
| 375 | 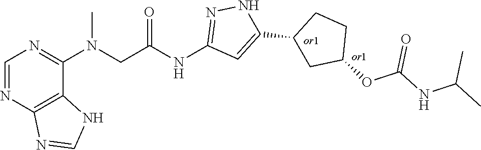 [SMILES: CC(C)NC(=O)O[C@H]1CC[C@@H](c2cc(NC(=O)CN(C)c3ncnc4nc[nH]c34)n[nH]2)C1] | A | |
| 376 | 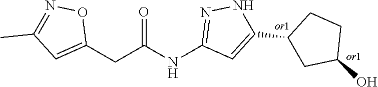 [SMILES: Cc1cc(CC(=O)Nc2cc([C@@H]3CC[C@@H](O)C3)[nH]n2)on1] | D | |
| 377 | 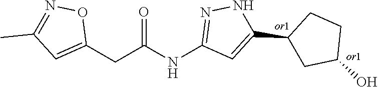 [SMILES: Cc1cc(CC(=O)Nc2cc([C@H]3CC[C@H](O)C3)[nH]n2)on1] | D | |
| 378 | 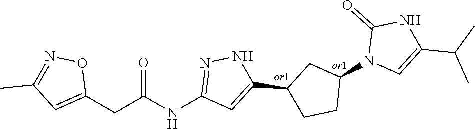 [SMILES: Cc1cc(CC(=O)Nc2cc([C@@H]3CC[C@H](n4cc(C(C)C)[nH]c4=O)C3)[nH]n2)on1] | D | |
| 379 | 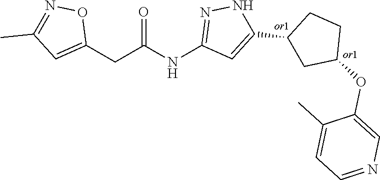 [SMILES: Cc1cc(CC(=O)Nc2cc([C@@H]3CC[C@H](Oc4cnccc4C)C3)[nH]n2)on1] | C | |
| 380 | 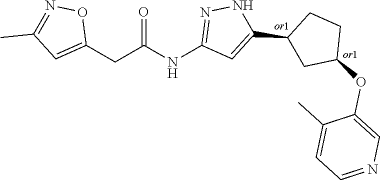 [SMILES: Cc1cc(CC(=O)Nc2cc([C@H]3CC[C@@H](Oc4cnccc4C)C3)[nH]n2)on1] | B | |
| 381 | 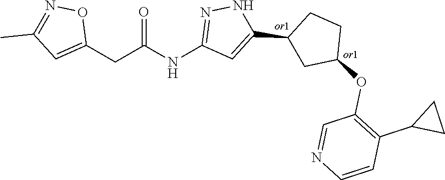 [SMILES: Cc1cc(CC(=O)Nc2cc([C@H]3CC[C@@H](Oc4cnccc4C4CC4)C3)[nH]n2)on1] | A | |
| 382 | 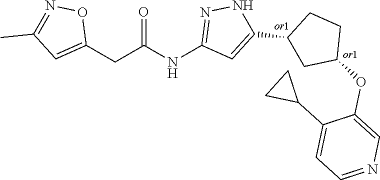 [SMILES: Cc1cc(CC(=O)Nc2cc([C@@H]3CC[C@H](Oc4cnccc4C4CC4)C3)[nH]n2)on1] | C | |
| 383 | 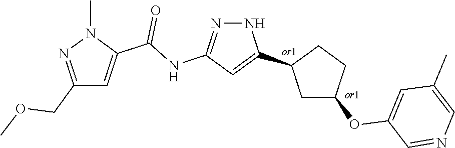 [SMILES: COCc1cc(C(=O)Nc2cc([C@H]3CC[C@@H](Oc4cncc(C)c4)C3)[nH]n2)n(C)n1] | D | |
| 384 | 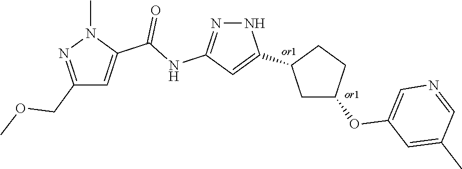 | D | |
| 385 | 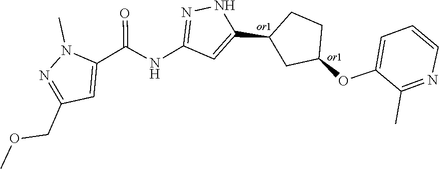 [SMILES: COCc1cc(C(=O)Nc2cc([C@H]3CC[C@@H](Oc4cccnc4C)C3)[nH]n2)n(C)n1] | D | |
| 386 | 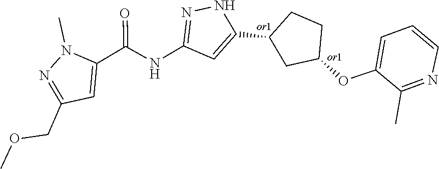 [SMILES: COCc1cc(C(=O)Nc2cc([C@@H]3CC[C@H](Oc4cccnc4C)C3)[nH]n2)n(C)n1] | C | |
| 387 | 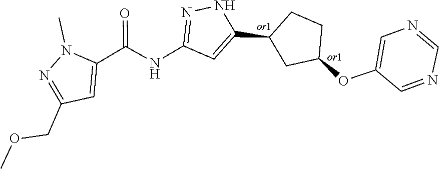 [SMILES: COCc1cc(C(=O)Nc2cc([C@H]3CC[C@@H](Oc4cncnc4)C3)[nH]n2)n(C)n1] | D | |
| 388 | 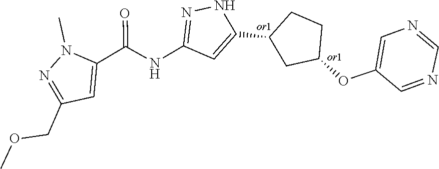 [SMILES: COCc1cc(C(=O)Nc2cc([C@@H]3CC[C@H](Oc4cncnc4)C3)[nH]n2)n(C)n1] | D | |
| 389 | 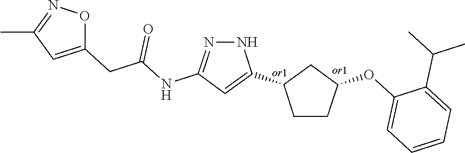 [SMILES: Cc1cc(CC(=O)Nc2cc([C@H]3CC[C@@H](Oc4ccccc4C(C)C)C3)[nH]n2)on1] | C | |
| 390 | 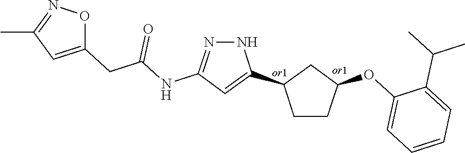 [SMILES: Cc1cc(CC(=O)Nc2cc([C@@H]3CC[C@H](Oc4ccccc4C(C)C)C3)[nH]n2)on1] | B | |
| 391 | 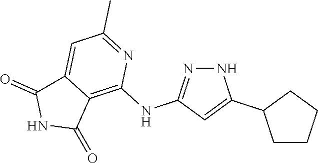 [SMILES: Cc1cc2c(c(Nc3cc(C4CCCC4)[nH]n3)n1)C(=O)NC2=O] | D | |
| 392 | 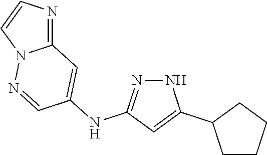 [SMILES: c1cn2ncc(Nc3cc(C4CCCC4)[nH]n3)cc2n1] | D | |
| 393 | 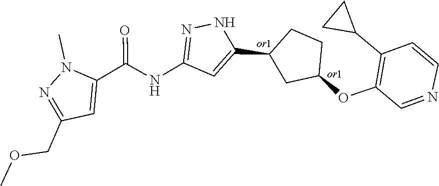 [SMILES: COCc1cc(C(=O)Nc2cc([C@H]3CC[C@@H](Oc4cnccc4C4CC4)C3)[nH]n2)n(C)n1] | A | |
| 394 | 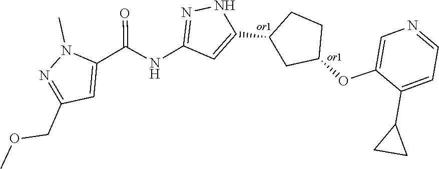 [SMILES: COCc1cc(C(=O)Nc2cc([C@@H]3CC[C@H](Oc4cnccc4C4CC4)C3)[nH]n2)n(C)n1] | B | |
| 395 | 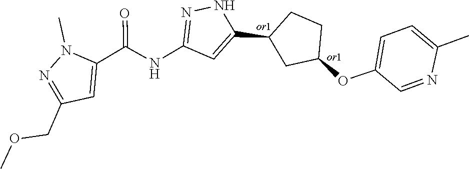 | D | |
| 396 | 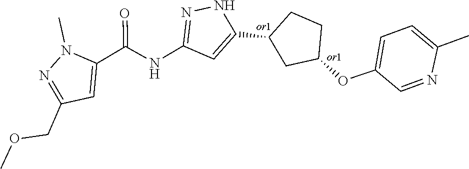 | D | |
| 397 | 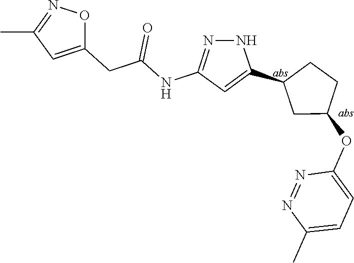 [SMILES: Cc1ccc(O[C@@H]2CC[C@H](c3cc(NC(=O)Cc4cc(C)no4)n[nH]3)C2)nn1] | C | |
| 398 | 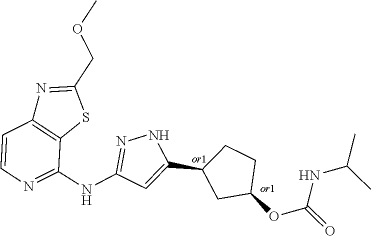 [SMILES: COCc1nc2ccnc(Nc3cc([C@H]4CC[C@@H](OC(=O)NC(C)C)C4)[nH]n3)c2s1] | A | |
| 399 | 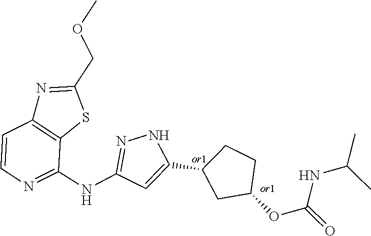 [SMILES: COCc1nc2ccnc(Nc3cc([C@@H]4CC[C@H](OC(=O)NC(C)C)C4)[nH]n3)c2s1] | A | |
| 400 | 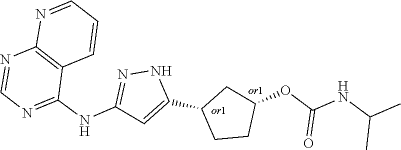 [SMILES: CC(C)NC(=O)O[C@@H]1CC[C@H](c2cc(Nc3ncnc4ncccc34)n[nH]2)C1] | A | |
| 401 | 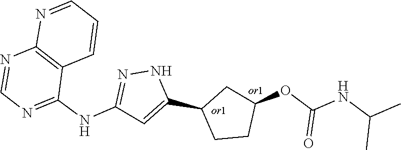 [SMILES: CC(C)NC(=O)O[C@H]1CC[C@@H](c2cc(Nc3ncnc4ncccc34)n[nH]2)C1] | A | |
| 402 | 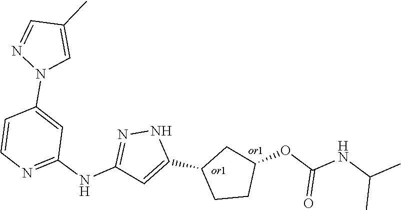 [SMILES: Cc1cnn(-c2ccnc(Nc3cc([C@H]4CC[C@@H](OC(=O)NC(C)C)C4)[nH]n3)c2)c1] | B | |
| 403 | 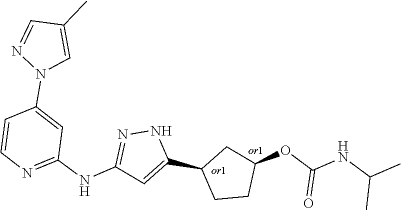 [SMILES: Cc1cnn(-c2ccnc(Nc3cc([C@@H]4CC[C@H](OC(=O)NC(C)C)C4)[nH]n3)c2)c1] | B | |
| 404 | 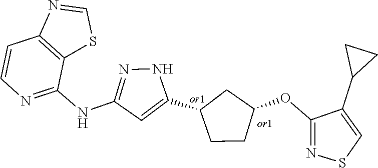 [SMILES: c1cc2ncsc2c(Nc2cc([C@H]3CC[C@@H](Oc4nscc4C4CC4)C3)[nH]n2)n1] | A | |
| 405 | 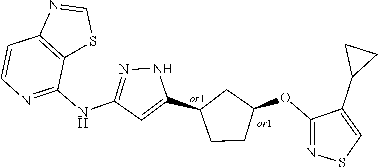 [SMILES: c1cc2ncsc2c(Nc2cc([C@@H]3CC[C@H](Oc4nscc4C4CC4)C3)[nH]n2)n1] | A | |
| 406 | 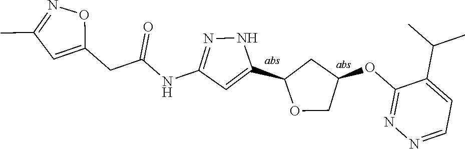 [SMILES: Cc1cc(CC(=O)Nc2cc([C@H]3C[C@@H](Oc4nnccc4C(C)C)CO3)[nH]n2)on1] | A | |
| 407 | 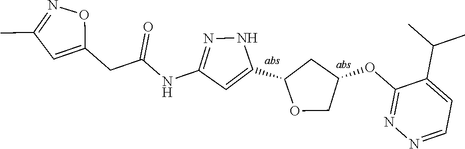 [SMILES: Cc1cc(CC(=O)Nc2cc([C@@H]3C[C@H](Oc4nnccc4C(C)C)CO3)[nH]n2)on1] | A | |
| 408 | 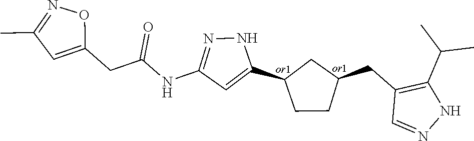 [SMILES: Cc1cc(CC(=O)Nc2cc([C@@H]3CC[C@H](Cc4cn[nH]c4C(C)C)C3)[nH]n2)on1] | C | |
| 409 | 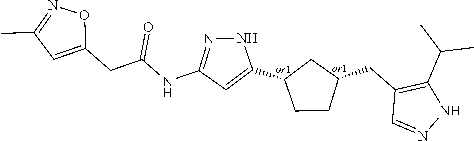 [SMILES: Cc1cc(CC(=O)Nc2cc([C@H]3CC[C@@H](Cc4cn[nH]c4C(C)C)C3)[nH]n2)on1] | B | |
| 410 | 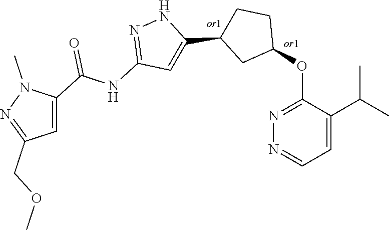 [SMILES: COCc1cc(C(=O)Nc2cc([C@H]3CC[C@@H](Oc4nnccc4C(C)C)C3)[nH]n2)n(C)n1] | A | |
| 411 | 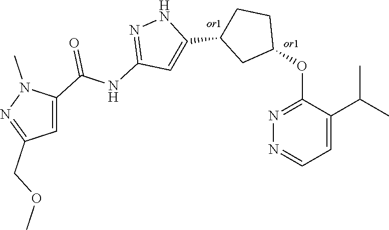 [SMILES: COCc1cc(C(=O)Nc2cc([C@@H]3CC[C@H](Oc4nnccc4C(C)C)C3)[nH]n2)n(C)n1] | A | |
| 412 | 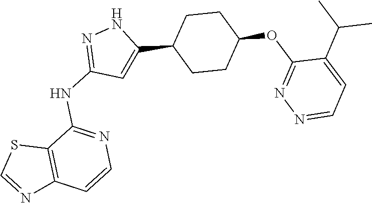 [SMILES: CC(C)c1ccnnc1O[C@H]1CC[C@@H](c2cc(Nc3nccc4ncsc34)n[nH]2)CC1] | A | |
| 413 | 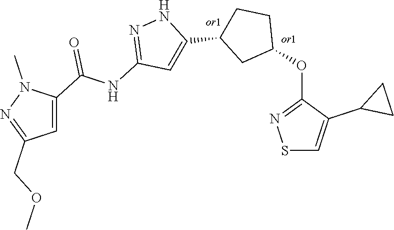 [SMILES: COCc1cc(C(=O)Nc2cc([C@@H]3CC[C@H](Oc4nscc4C4CC4)C3)[nH]n2)n(C)n1] | A | |
| 414 | 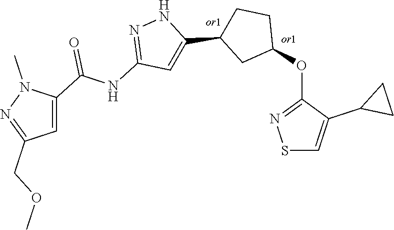 [SMILES: COCc1cc(C(=O)Nc2cc([C@H]3CC[C@@H](Oc4nscc4C4CC4)C3)[nH]n2)n(C)n1] | A | |
| 415 | 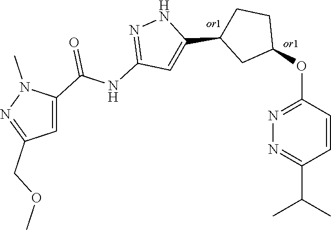 [SMILES: COCc1cc(C(=O)Nc2cc([C@H]3CC[C@@H](Oc4ccc(C(C)C)nn4)C3)[nH]n2)n(C)n1] | C | |
| 416 | 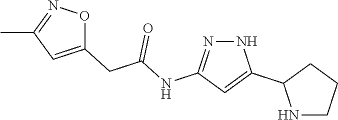 [SMILES: Cc1cc(CC(=O)Nc2cc(C3CCCN3)[nH]n2)on1] | D | |
| 417 | 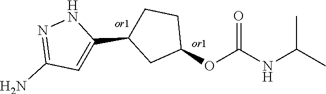 [SMILES: CC(C)NC(=O)O[C@@H]1CC[C@H](c2cc(N)n[nH]2)C1] | D | |
| 418 | 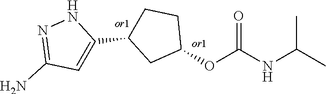 [SMILES: CC(C)NC(=O)O[C@H]1CC[C@@H](c2cc(N)n[nH]2)C1] | D | |
| 419 | 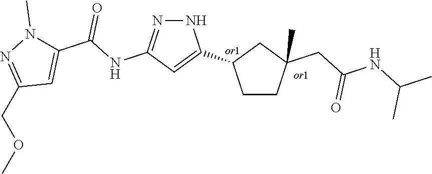 [SMILES: COCc1cc(C(=O)Nc2cc([C@H]3CC[C@@](C)(CC(=O)NC(C)C)C3)[nH]n2)n(C)n1] | D | |
| 420 | 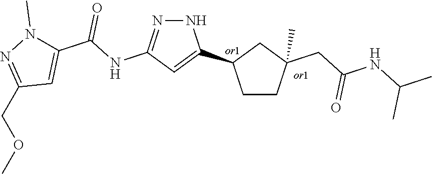 [SMILES: COCc1cc(C(=O)Nc2cc([C@@H]3CC[C@](C)(CC(=O)NC(C)C)C3)[nH]n2)n(C)n1] | D | |
| 421 | 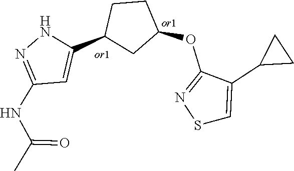 [SMILES: CC(=O)Nc1cc([C@H]2CC[C@@H](Oc3nscc3C3CC3)C2)[nH]n1] | A | |
| 422 | 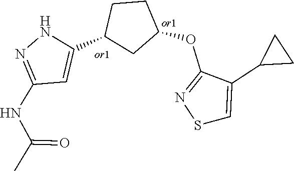 [SMILES: CC(=O)Nc1cc([C@@H]2CC[C@H](Oc3nscc3C3CC3)C2)[nH]n1] | A | |
| 423 | 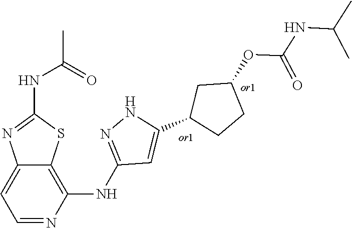 [SMILES: CC(=O)Nc1nc2ccnc(Nc3cc([C@H]4CC[C@@H](OC(=O)NC(C)C)C4)[nH]n3)c2s1] | A | |
| 424 | 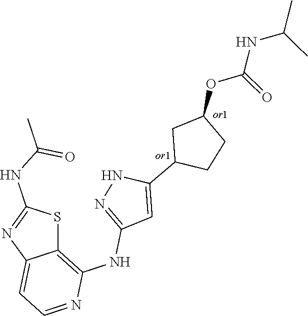 [SMILES: CC(=O)Nc1nc2ccnc(Nc3cc(C4CC[C@H](OC(=O)NC(C)C)C4)[nH]n3)c2s1] | A | |
| 425 | 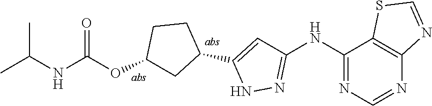 [SMILES: CC(C)NC(=O)O[C@@H]1CC[C@H](c2cc(Nc3ncnc4ncsc34)n[nH]2)C1] | A | |
| 426 | 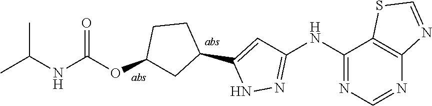 [SMILES: CC(C)NC(=O)O[C@H]1CC[C@@H](c2cc(Nc3ncnc4ncsc34)n[nH]2)C1] | A | |
| 427 | 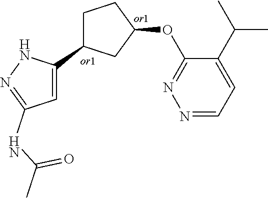 [SMILES: CC(=O)Nc1cc([C@H]2CC[C@@H](Oc3nnccc3C(C)C)C2)[nH]n1] | A | |
| 428 | 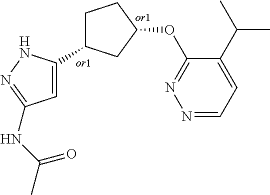 [SMILES: CC(=O)Nc1cc([C@@H]2CC[C@H](Oc3nnccc3C(C)C)C2)[nH]n1] | A | |
| 429 | 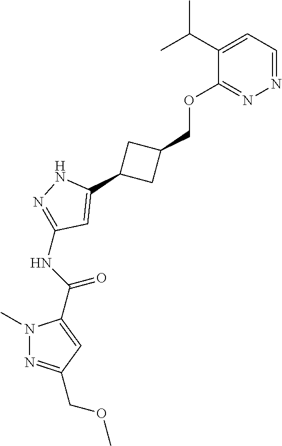 [SMILES: COCc1cc(C(=O)Nc2cc([C@H]3C[C@@H](COc4nnccc4C(C)C)C3)[nH]n2)n(C)n1] | A | |
| 430 | 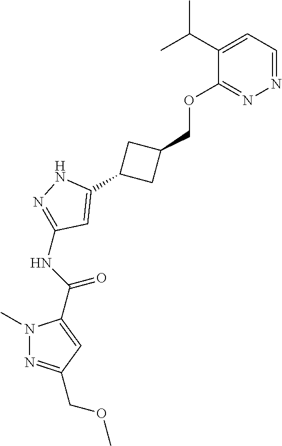 [SMILES: COCc1cc(C(=O)Nc2cc([C@H]3C[C@H](COc4nnccc4C(C)C)C3)[nH]n2)n(C)n1] | D | |
| 431 | 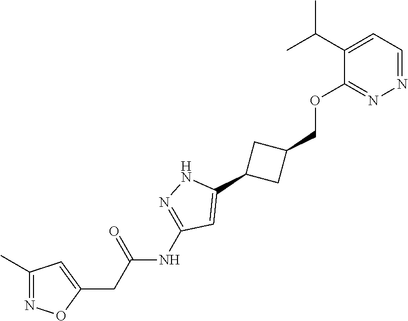 [SMILES: Cc1cc(CC(=O)Nc2cc([C@H]3C[C@@H](COc4nnccc4C(C)C)C3)[nH]n2)on1] | A | |
| 432 | 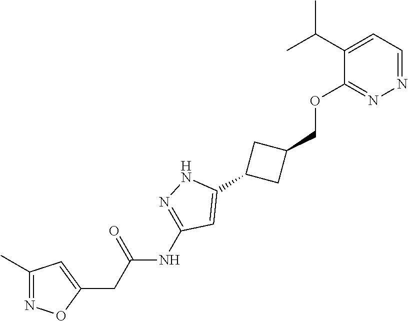 [SMILES: Cc1cc(CC(=O)Nc2cc([C@H]3C[C@H](COc4nnccc4C(C)C)C3)[nH]n2)on1] | B | |
| 433 | 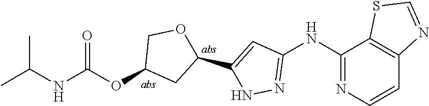 [SMILES: CC(C)NC(=O)O[C@H]1CO[C@@H](c2cc(Nc3nccc4ncsc34)n[nH]2)C1] | A | |
| 434 | 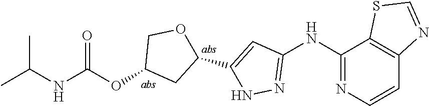 [SMILES: CC(C)NC(=O)O[C@@H]1CO[C@H](c2cc(Nc3nccc4ncsc34)n[nH]2)C1] | B | |
| 435 | 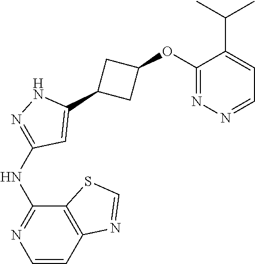 [SMILES: CC(C)c1ccnnc1O[C@H]1C[C@@H](c2cc(Nc3nccc4ncsc34)n[nH]2)C1] | A | |
| 436 | 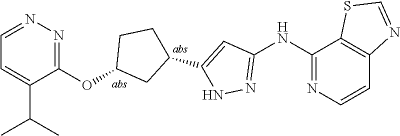 [SMILES: CC(C)c1ccnnc1O[C@@H]1CC[C@H](c2cc(Nc3nccc4ncsc34)n[nH]2)C1] | A | |
| 437 | 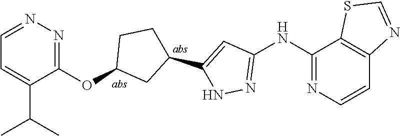 [SMILES: CC(C)c1ccnnc1O[C@H]1CC[C@@H](c2cc(Nc3nccc4ncsc34)n[nH]2)C1] | A | |
| 438 | 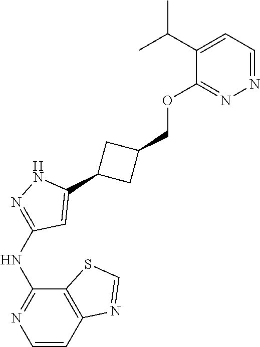 [SMILES: CC(C)c1ccnnc1OC[C@H]1C[C@@H](c2cc(Nc3nccc4ncsc34)n[nH]2)C1] | A | |
| 439 | 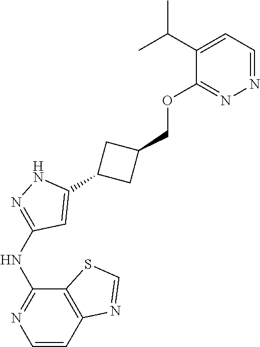 [SMILES: CC(C)c1ccnnc1OC[C@H]1C[C@H](c2cc(Nc3nccc4ncsc34)n[nH]2)C1] | D | |
| 440 | 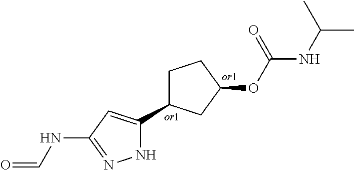 [SMILES: CC(C)NC(=O)O[C@@H]1CC[C@H](c2cc(NC=O)n[nH]2)C1] | C | |
| 441 | 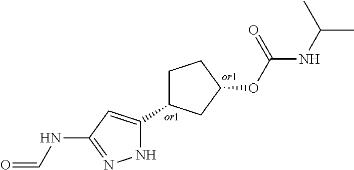 [SMILES: CC(C)NC(=O)O[C@H]1CC[C@@H](c2cc(NC=O)n[nH]2)C1] | C | |
| 442 | 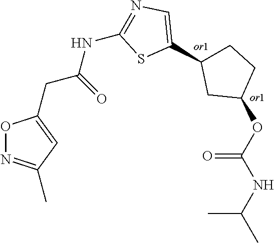 [SMILES: Cc1cc(CC(=O)Nc2ncc([C@H]3CC[C@@H](OC(=O)NC(C)C)C3)s2)on1] | A | |
| 443 | 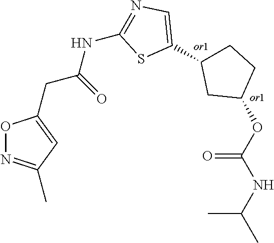 [SMILES: Cc1cc(CC(=O)Nc2ncc([C@@H]3CC[C@H](OC(=O)NC(C)C)C3)s2)on1] | A | |
| 444 | 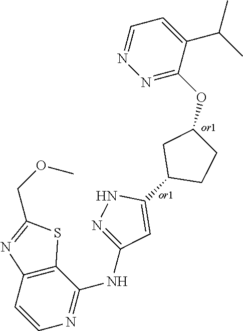 [SMILES: COCc1nc2ccnc(Nc3cc([C@H]4CC[C@@H](Oc5nnccc5C(C)C)C4)[nH]n3)c2s1] | A | |
| 445 | 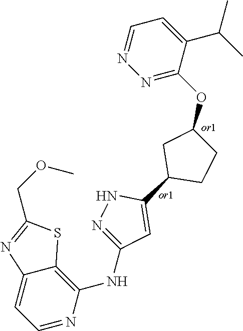 [SMILES: COCc1nc2ccnc(Nc3cc([C@@H]4CC[C@H](Oc5nnccc5C(C)C)C4)[nH]n3)c2s1] | A | |
| 446 | 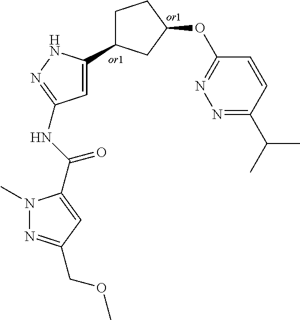 [SMILES: COCc1cc(C(=O)Nc2cc([C@H]3CC[C@@H](Oc4ccc(C(C)C)nn4)C3)[nH]n2)n(C)n1] | | |
| 447 | 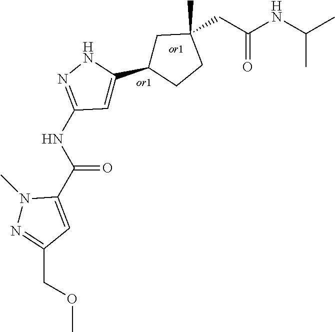 [SMILES: COCc1cc(C(=O)Nc2cc([C@@H]3CC[C@@](C)(CC(=O)NC(C)C)C3)[nH]n2)n(C)n1] | D | |
| 448 | 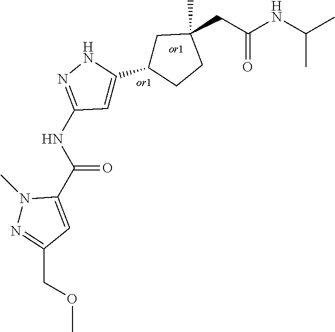 [SMILES: COCc1cc(C(=O)Nc2cc([C@H]3CC[C@](C)(CC(=O)NC(C)C)C3)[nH]n2)n(C)n1] | D | |
| 449 | 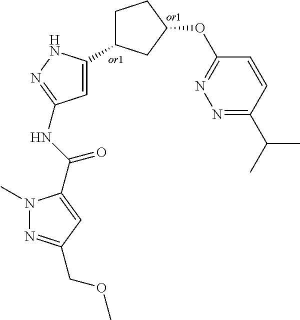 [SMILES: COCc1cc(C(=O)Nc2cc([C@@H]3CC[C@H](Oc4ccc(C(C)C)nn4)C3)[nH]n2)n(C)n1] | D | |
| 450 | 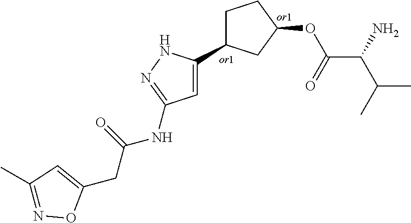 [SMILES: Cc1cc(CC(=O)Nc2cc([C@H]3CC[C@@H](OC(=O)[C@H](N)C(C)C)C3)[nH]n2)on1] | A | |
| 451 | 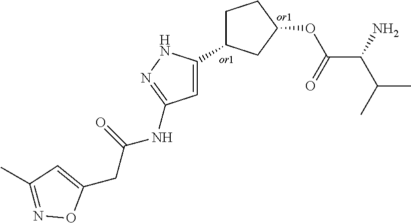 [SMILES: Cc1cc(CC(=O)Nc2cc([C@@H]3CC[C@H](OC(=O)[C@H](N)C(C)C)C3)[nH]n2)on1] | B | |
| 452 | 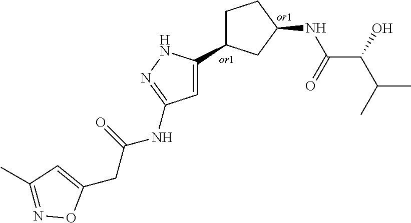 [SMILES: Cc1cc(CC(=O)Nc2cc([C@H]3CC[C@@H](NC(=O)[C@H](O)C(C)C)C3)[nH]n2)on1] | D | |
| 453 | 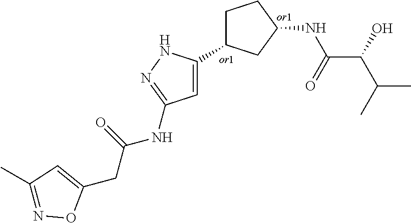 [SMILES: Cc1cc(CC(=O)Nc2cc([C@@H]3CC[C@H](NC(=O)[C@H](O)C(C)C)C3)[nH]n2)on1] | D | |
| 454 | 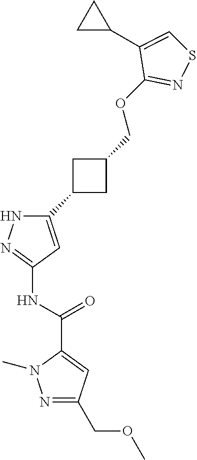 [SMILES: COCc1cc(C(=O)Nc2cc([C@H]3C[C@@H](COc4nscc4C4CC4)C3)[nH]n2)n(C)n1] | A | |
| 455 | 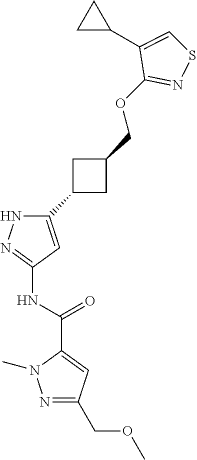 [SMILES: COCc1cc(C(=O)Nc2cc([C@H]3C[C@H](COc4nscc4C4CC4)C3)[nH]n2)n(C)n1] | D | |
| 456 | 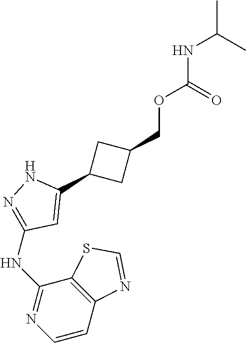 [SMILES: CC(C)NC(=O)OC[C@H]1C[C@@H](c2cc(Nc3nccc4ncsc34)n[nH]2)C1] | A | |
| 457 | 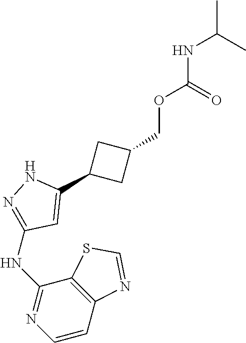 [SMILES: CC(C)NC(=O)OC[C@H]1C[C@H](c2cc(Nc3nccc4ncsc34)n[nH]2)C1] | C | |
| 458 | 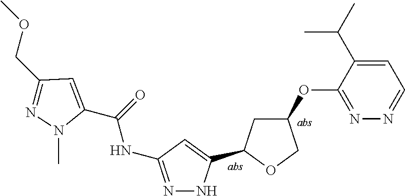 [SMILES: COCc1cc(C(=O)Nc2cc([C@H]3C[C@@H](Oc4nnccc4C(C)C)CO3)[nH]n2)n(C)n1] | A | |
| 459 | 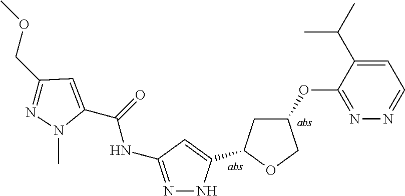 [SMILES: COCc1cc(C(=O)Nc2cc([C@@H]3C[C@H](Oc4nnccc4C(C)C)CO3)[nH]n2)n(C)n1] | A | |
| 460 | 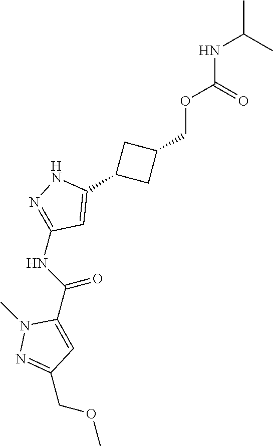 [SMILES: COCc1cc(C(=O)Nc2cc([C@H]3C[C@@H](COC(=O)NC(C)C)C3)[nH]n2)n(C)n1] | A | |
| 461 | 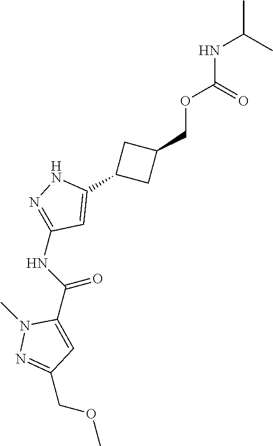 [SMILES: COCc1cc(C(=O)Nc2cc([C@H]3C[C@H](COC(=O)NC(C)C)C3)[nH]n2)n(C)n1] | D | |
| 462 | 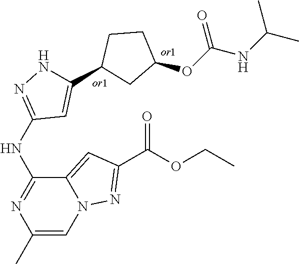 [SMILES: CCOC(=O)c1cc2c(Nc3cc([C@H]4CC[C@@H](OC(=O)NC(C)C)C4)[nH]n3)nc(C)cn2n1] | A | |
| 463 | 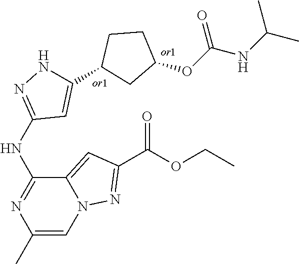 [SMILES: CCOC(=O)c1cc2c(Nc3cc([C@@H]4CC[C@H](OC(=O)NC(C)C)C4)[nH]n3)nc(C)cn2n1] | A | |
| 464 | 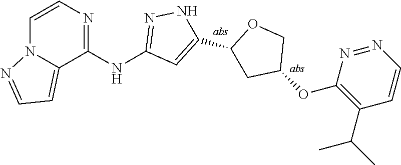 [SMILES: CC(C)c1ccnnc1O[C@H]1CO[C@@H](c2cc(Nc3nccn4nccc34)n[nH]2)C1] | A | |
| 465 | 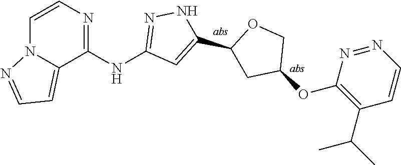 [SMILES: CC(C)c1ccnnc1O[C@@H]1CO[C@H](c2cc(Nc3nccn4nccc34)n[nH]2)C1] | A | |
| 466 | 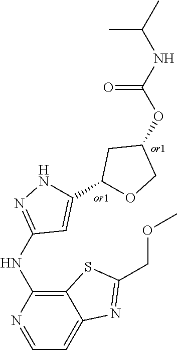 [SMILES: COCc1nc2ccnc(Nc3cc([C@@H]4C[C@H](OC(=O)NC(C)C)CO4)[nH]n3)c2s1] | A | |
| 467 | 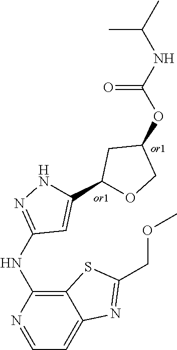 [SMILES: COCc1nc2ccnc(Nc3cc([C@H]4C[C@@H](OC(=O)NC(C)C)CO4)[nH]n3)c2s1] | B | |
| 468 | 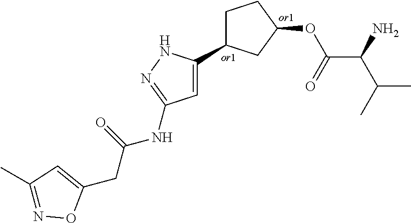 [SMILES: Cc1cc(CC(=O)Nc2cc([C@H]3CC[C@@H](OC(=O)[C@@H](N)C(C)C)C3)[nH]n2)on1] | B | |
| 469 | 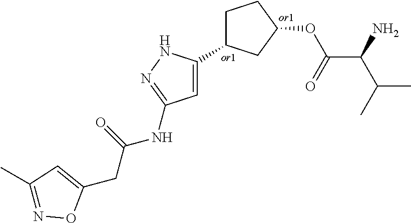 [SMILES: Cc1cc(CC(=O)Nc2cc([C@@H]3CC[C@H](OC(=O)[C@@H](N)C(C)C)C3)[nH]n2)on1] | C | |
| 470 | 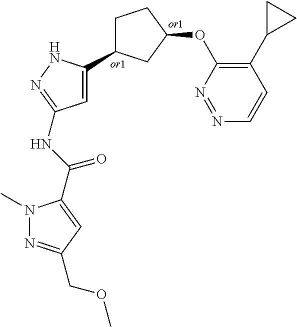 [SMILES: COCc1cc(C(=O)Nc2cc([C@H]3CC[C@@H](Oc4nnccc4C4CC4)C3)[nH]n2)n(C)n1] | A | |
| 471 | 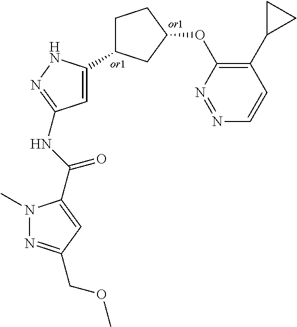 [SMILES: COCc1cc(C(=O)Nc2cc([C@@H]3CC[C@H](Oc4nnccc4C4CC4)C3)[nH]n2)n(C)n1] | A | |
| 472 | 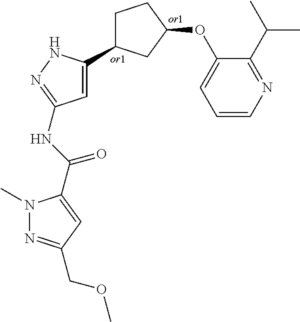 [SMILES: COCc1cc(C(=O)Nc2cc([C@H]3CC[C@@H](Oc4cccnc4C(C)C)C3)[nH]n2)n(C)n1] | B | |
| 473 | 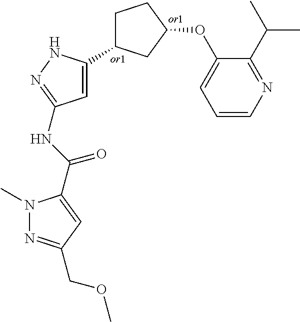 [SMILES: COCc1cc(C(=O)Nc2cc([C@@H]3CC[C@H](Oc4cccnc4C(C)C)C3)[nH]n2)n(C)n1] | B | |
| 474 | 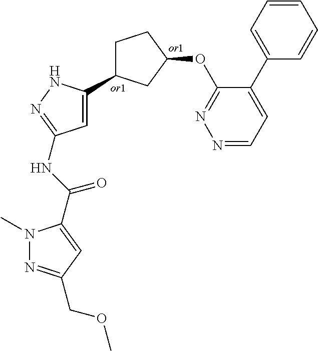 [SMILES: COCc1cc(C(=O)Nc2cc([C@H]3CC[C@@H](Oc4nnccc4-c4ccccc4)C3)[nH]n2)n(C)n1] | B | |
| 475 | 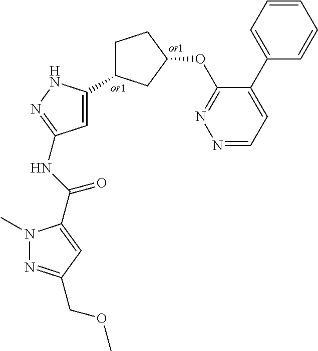 [SMILES: COCc1cc(C(=O)Nc2cc([C@@H]3CC[C@H](Oc4nnccc4-c4ccccc4)C3)[nH]n2)n(C)n1] | A | |
| 476 | 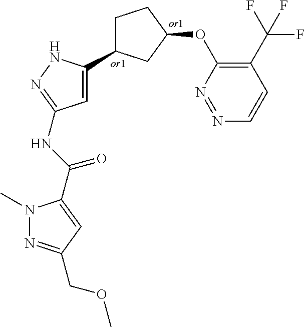 [SMILES: COCc1cc(C(=O)Nc2cc([C@H]3CC[C@@H](Oc4nnccc4C(F)(F)F)C3)[nH]n2)n(C)n1] | A | |
| 477 | 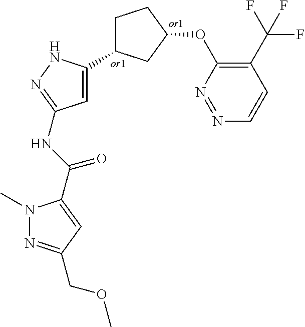 [SMILES: COCc1cc(C(=O)Nc2cc([C@@H]3CC[C@H](Oc4nnccc4C(F)(F)F)C3)[nH]n2)n(C)n1] | A | |
| 478 | 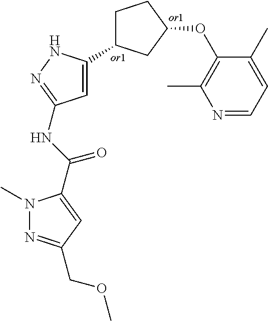 [SMILES: COCc1cc(C(=O)Nc2cc([C@@H]3CC[C@H](Oc4c(C)ccnc4C)C3)[nH]n2)n(C)n1] | D | |
| 479 | 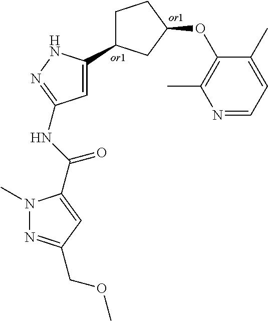 [SMILES: COCc1cc(C(=O)Nc2cc([C@H]3CC[C@@H](Oc4c(C)ccnc4C)C3)[nH]n2)n(C)n1] | D | |
| 480 | 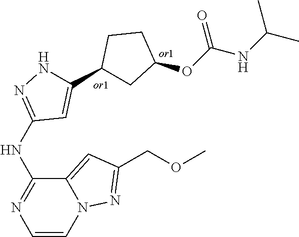 [SMILES: COCc1cc2c(Nc3cc([C@H]4CC[C@@H](OC(=O)NC(C)C)C4)[nH]n3)nccn2n1] | A | |
| 481 | 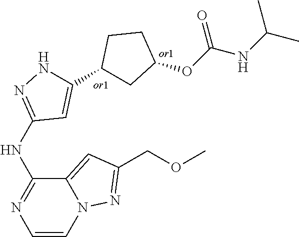 [SMILES: COCc1cc2c(Nc3cc([C@@H]4CC[C@H](OC(=O)NC(C)C)C4)[nH]n3)nccn2n1] | A | |
| 482 | 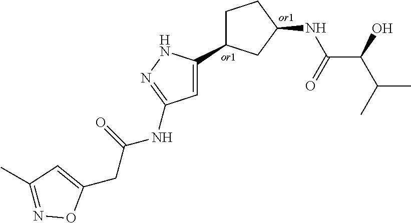 [SMILES: Cc1cc(CC(=O)Nc2cc([C@H]3CC[C@@H](NC(=O)[C@@H](O)C(C)C)C3)[nH]n2)on1] | D | |
| 483 | 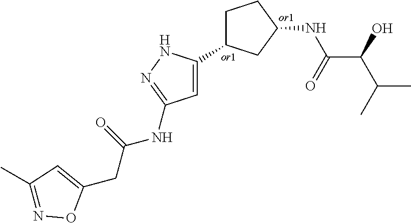 [SMILES: Cc1cc(CC(=O)Nc2cc([C@@H]3CC[C@H](NC(=O)[C@@H](O)C(C)C)C3)[nH]n2)on1] | C | |
| 484 | 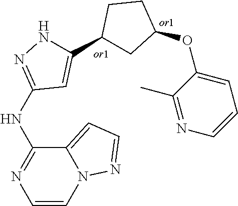 [SMILES: Cc1ncccc1O[C@@H]1CC[C@H](c2cc(Nc3nccn4nccc34)n[nH]2)C1] | C | |
| 485 | 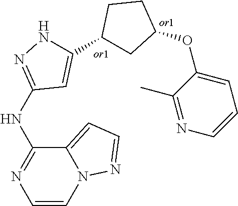 [SMILES: Cc1ncccc1O[C@H]1CC[C@@H](c2cc(Nc3nccn4nccc34)n[nH]2)C1] | A | |
| 486 | 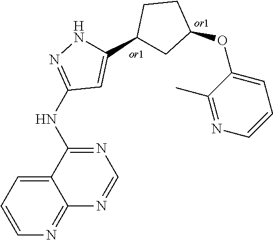 [SMILES: Cc1ncccc1O[C@@H]1CC[C@H](c2cc(Nc3ncnc4ncccc34)n[nH]2)C1] | D | |
| 487 | 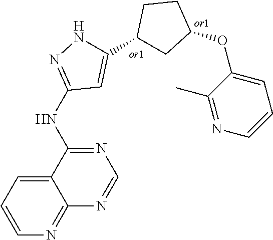 [SMILES: Cc1ncccc1O[C@H]1CC[C@@H](c2cc(Nc3ncnc4ncccc34)n[nH]2)C1] | A | |
| 488 | 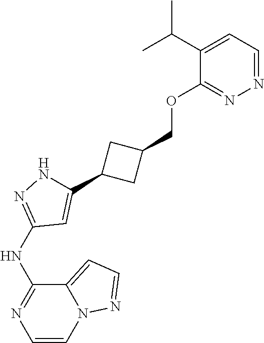 [SMILES: CC(C)c1ccnnc1OC[C@H]1C[C@@H](c2cc(Nc3nccn4nccc34)n[nH]2)C1] | A | |
| 489 | 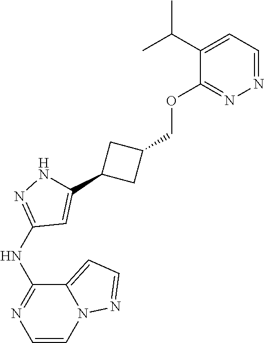 [SMILES: CC(C)c1ccnnc1OC[C@H]1C[C@H](c2cc(Nc3nccn4nccc34)n[nH]2)C1] | C | |
| 490 | 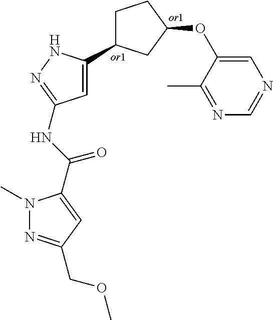 [SMILES: COCc1cc(C(=O)Nc2cc([C@H]3CC[C@@H](Oc4cncnc4C)C3)[nH]n2)n(C)n1] | D | |
| 491 | 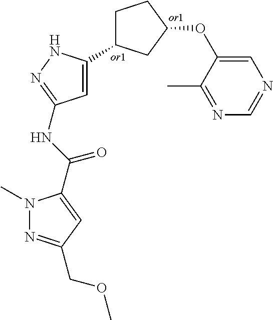 [SMILES: COCc1cc(C(=O)Nc2cc([C@@H]3CC[C@H](Oc4cncnc4C)C3)[nH]n2)n(C)n1] | D | |
| 492 | 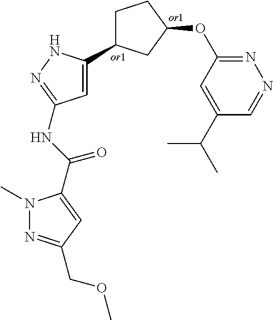 [SMILES: COCc1cc(C(=O)Nc2cc([C@H]3CC[C@@H](Oc4cc(C(C)C)cnn4)C3)[nH]n2)n(C)n1] | A | |
| 493 | 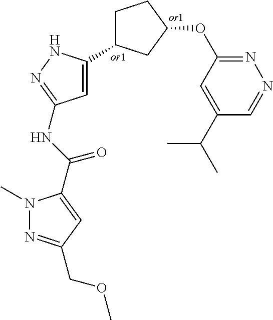 [SMILES: COCc1cc(C(=O)Nc2cc([C@@H]3CC[C@H](Oc4cc(C(C)C)cnn4)C3)[nH]n2)n(C)n1] | B | |
| 494 | 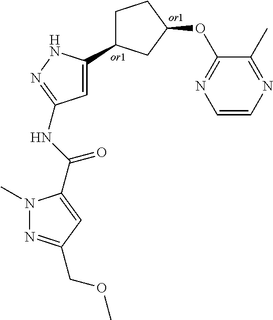 | C | |
| 495 | 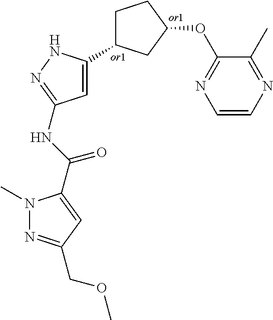 | C | |
| 496 | 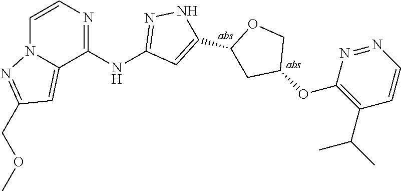 [SMILES: COCc1cc2c(Nc3cc([C@H]4C[C@@H](Oc5nnccc5C(C)C)CO4)[nH]n3)nccn2n1] | A | |
| 497 | 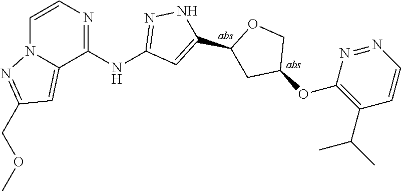 [SMILES: COCc1cc2c(Nc3cc([C@@H]4C[C@H](Oc5nnccc5C(C)C)CO4)[nH]n3)nccn2n1] | A | |
| 498 | 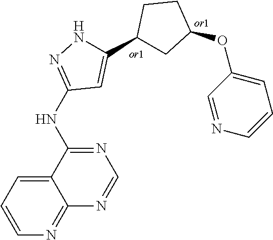 [SMILES: c1cncc(O[C@@H]2CC[C@H](c3cc(Nc4ncnc5ncccc45)n[nH]3)C2)c1] | D | |
| 499 | 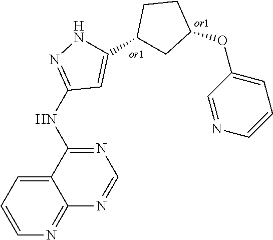 [SMILES: c1cncc(O[C@H]2CC[C@@H](c3cc(Nc4ncnc5ncccc45)n[nH]3)C2)c1] | C | |
| 500 | 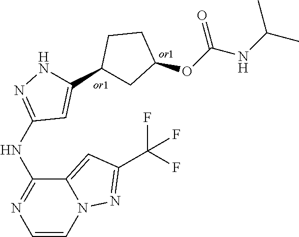 [SMILES: CC(C)NC(=O)O[C@@H]1CC[C@H](c2cc(Nc3nccn4nc(C(F)(F)F)cc34)n[nH]2)C1] | A | |
| 501 | 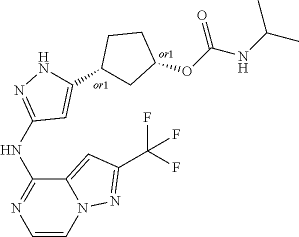 [SMILES: CC(C)NC(=O)O[C@H]1CC[C@@H](c2cc(Nc3nccn4nc(C(F)(F)F)cc34)n[nH]2)C1] | A | |
| 502 | 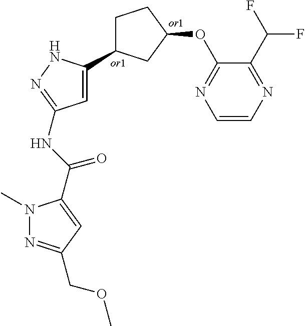 [SMILES: COCc1cc(C(=O)Nc2cc([C@H]3CC[C@@H](Oc4nccnc4C(F)F)C3)[nH]n2)n(C)n1] | D | |
| 503 | 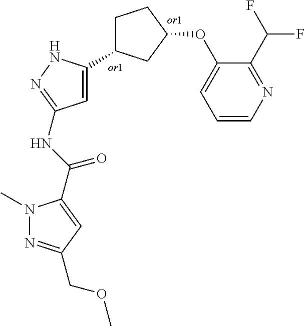 [SMILES: COCc1cc(C(=O)Nc2cc([C@@H]3CC[C@H](Oc4cccnc4C(F)F)C3)[nH]n2)n(C)n1] | D | |
| 504 | 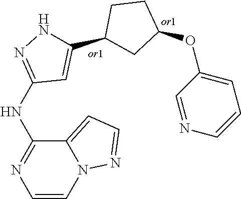 [SMILES: c1cncc(O[C@@H]2CC[C@H](c3cc(Nc4nccn5nccc45)n[nH]3)C2)c1] | D | |
| 505 | 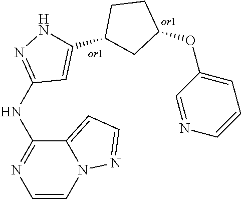 [SMILES: c1cncc(O[C@H]2CC[C@@H](c3cc(Nc4nccn5nccc45)n[nH]3)C2)c1] | A | |
| 506 | 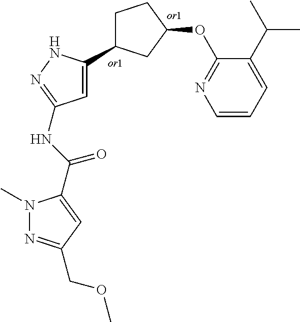 [SMILES: COCc1cc(C(=O)Nc2cc([C@H]3CC[C@@H](Oc4ncccc4C(C)C)C3)[nH]n2)n(C)n1] | A | |
| 507 | 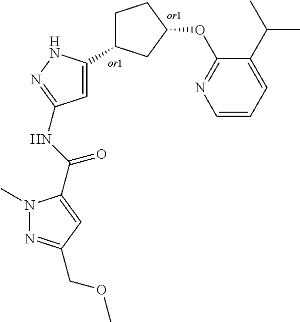 [SMILES: COCc1cc(C(=O)Nc2cc([C@@H]3CC[C@H](Oc4ncccc4C(C)C)C3)[nH]n2)n(C)n1] | A | |
| 508 | 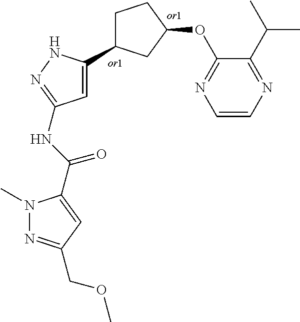 [SMILES: COCc1cc(C(=O)Nc2cc([C@H]3CC[C@@H](Oc4nccnc4C(C)C)C3)[nH]n2)n(C)n1] | A | |
| 509 | 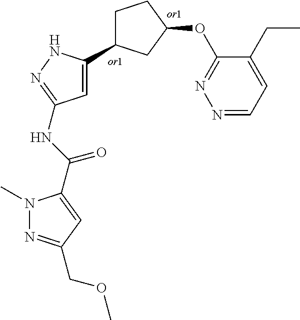 [SMILES: CCc1ccnnc1O[C@@H]1CC[C@H](c2cc(NC(=O)c3cc(COC)nn3C)n[nH]2)C1] | A | |
| 510 | 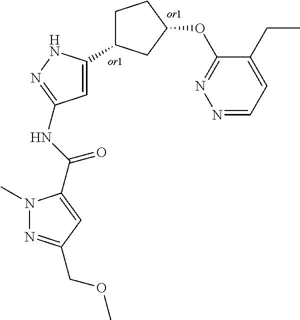 [SMILES: CCc1ccnnc1O[C@H]1CC[C@@H](c2cc(NC(=O)c3cc(COC)nn3C)n[nH]2)C1] | A | |
| 511 | 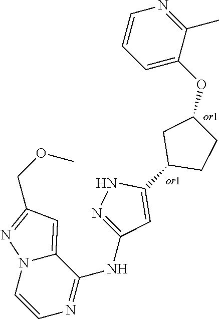 [SMILES: COCc1cc2c(Nc3cc([C@H]4CC[C@@H](Oc5cccnc5C)C4)[nH]n3)nccn2n1] | D | |
| 512 | 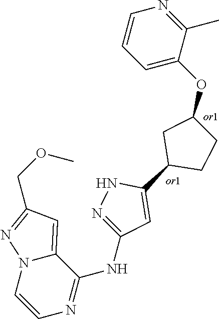 [SMILES: COCc1cc2c(Nc3cc([C@@H]4CC[C@H](Oc5cccnc5C)C4)[nH]n3)nccn2n1] | B | |
| 513 | 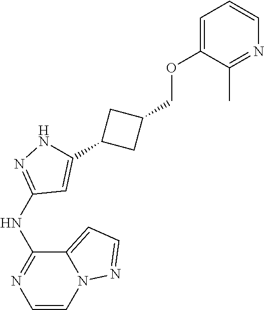 [SMILES: Cc1ncccc1OC[C@H]1C[C@@H](c2cc(Nc3nccn4nccc34)n[nH]2)C1] | B | |
| 514 | 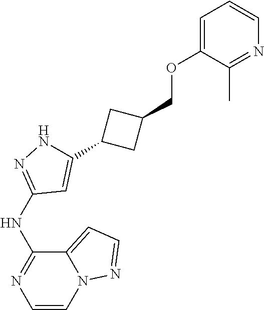 [SMILES: Cc1ncccc1OC[C@H]1C[C@H](c2cc(Nc3nccn4nccc34)n[nH]2)C1] | D | |
| 515 | 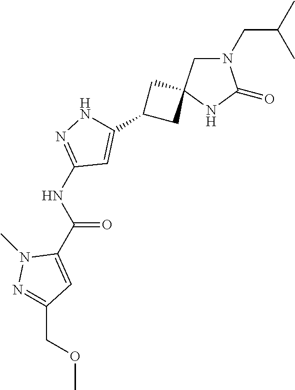 [SMILES: COCc1cc(C(=O)Nc2cc([C@H]3C[C@]4(CN(CC(C)C)C(=O)N4)C3)[nH]n2)n(C)n1] | D | |
| 516 | 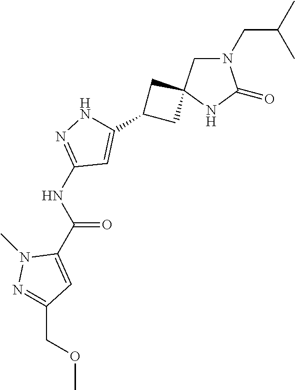 [SMILES: COCc1cc(C(=O)Nc2cc([C@H]3C[C@@]4(CN(CC(C)C)C(=O)N4)C3)[nH]n2)n(C)n1] | C | |
| 517 | 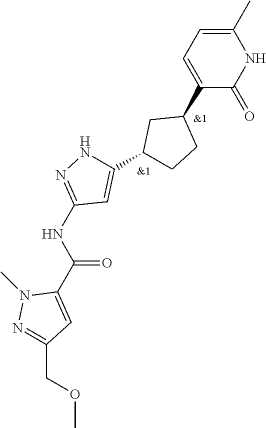 | D | |
| 518 | 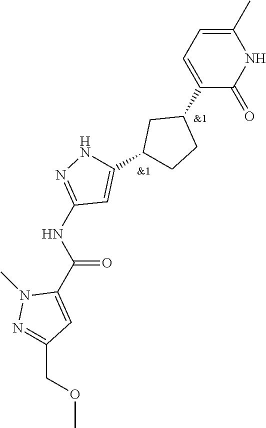 | C | |
| 519 | 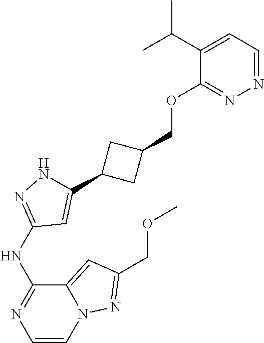 [SMILES: COCc1cc2c(Nc3cc([C@H]4C[C@@H](COc5nnccc5C(C)C)C4)[nH]n3)nccn2n1] | A | |
| 520 | 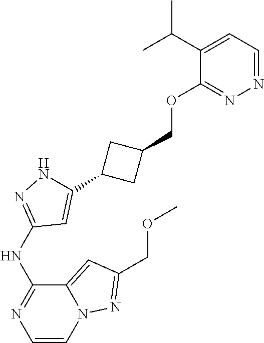 [SMILES: COCc1cc2c(Nc3cc([C@H]4C[C@H](COc5nnccc5C(C)C)C4)[nH]n3)nccn2n1] | D | |
| 521 | 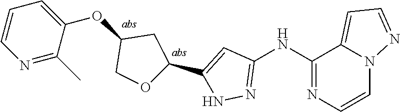 [SMILES: Cc1ncccc1O[C@@H]1CO[C@H](c2cc(Nc3nccn4nccc34)n[nH]2)C1] | D | |
| 522 | 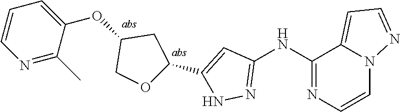 [SMILES: Cc1ncccc1O[C@H]1CO[C@@H](c2cc(Nc3nccn4nccc34)n[nH]2)C1] | C | |
| 523 | 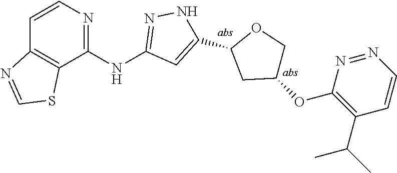 [SMILES: CC(C)c1ccnnc1O[C@H]1CO[C@@H](c2cc(Nc3nccc4ncsc34)n[nH]2)C1] | A | |
| 524 | 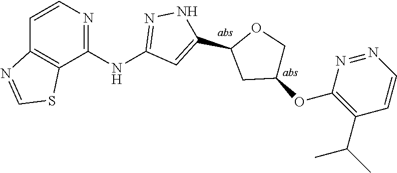 [SMILES: CC(C)c1ccnnc1O[C@@H]1CO[C@H](c2cc(Nc3nccc4ncsc34)n[nH]2)C1] | A | |
| 525 | 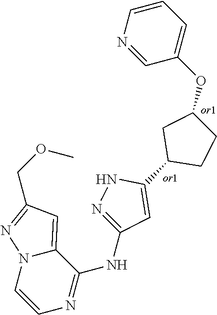 [SMILES: COCc1cc2c(Nc3cc([C@H]4CC[C@@H](Oc5cccnc5)C4)[nH]n3)nccn2n1] | D | |
| 526 | 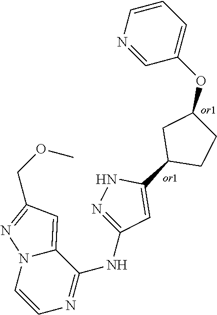 [SMILES: COCc1cc2c(Nc3cc([C@@H]4CC[C@H](Oc5cccnc5)C4)[nH]n3)nccn2n1] | B | |
| 527 | 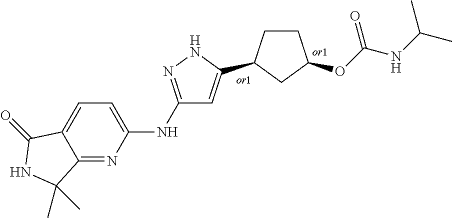 [SMILES: CC(C)NC(=O)O[C@@H]1CC[C@H](c2cc(Nc3ccc4c(n3)C(C)(C)NC4=O)n[nH]2)C1] | A | |
| 528 | 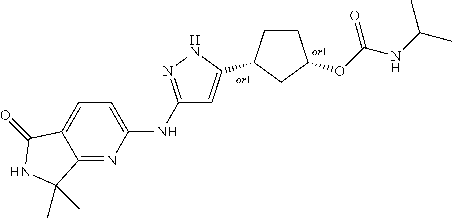 [SMILES: CC(C)NC(=O)O[C@H]1CC[C@@H](c2cc(Nc3ccc4c(n3)C(C)(C)NC4=O)n[nH]2)C1] | A | |
| 529 | 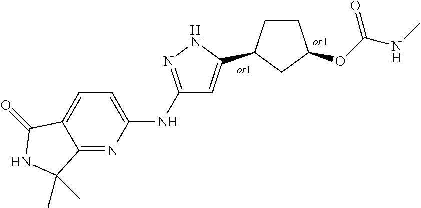 [SMILES: CNC(=O)O[C@@H]1CC[C@H](c2cc(Nc3ccc4c(n3)C(C)(C)NC4=O)n[nH]2)C1] | A | |
| 530 | 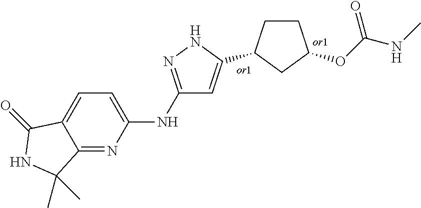 [SMILES: CNC(=O)O[C@H]1CC[C@@H](c2cc(Nc3ccc4c(n3)C(C)(C)NC4=O)n[nH]2)C1] | A | |
| 531 | 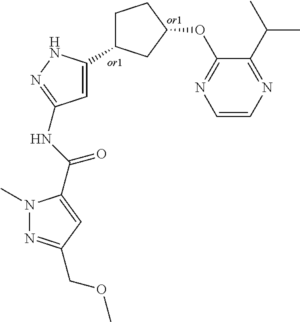 [SMILES: COCc1cc(C(=O)Nc2cc([C@@H]3CC[C@H](Oc4nccnc4C(C)C)C3)[nH]n2)n(C)n1] | A | |
| 532 | 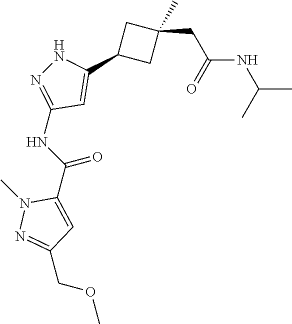 [SMILES: COCc1cc(C(=O)Nc2cc([C@H]3C[C@@](C)(CC(=O)NC(C)C)C3)[nH]n2)n(C)n1] | D | |
| 533 | 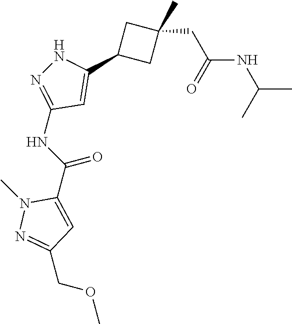 [SMILES: COCc1cc(C(=O)Nc2cc([C@H]3C[C@](C)(CC(=O)NC(C)C)C3)[nH]n2)n(C)n1] | D | |
| 534 | 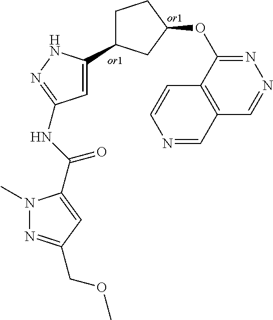 [SMILES: COCc1cc(C(=O)Nc2cc([C@H]3CC[C@@H](Oc4nncc5cnccc45)C3)[nH]n2)n(C)n1] | A | |
| 535 | 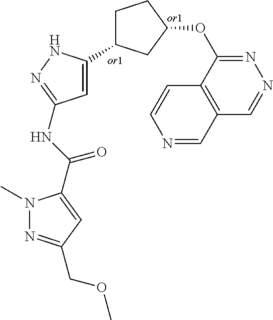 [SMILES: COCc1cc(C(=O)Nc2cc([C@@H]3CC[C@H](Oc4nncc5cnccc45)C3)[nH]n2)n(C)n1] | C | |
| 536 | 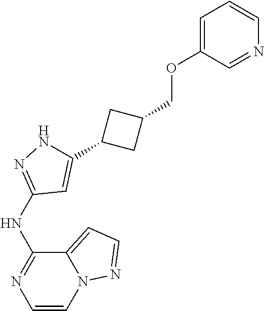 [SMILES: c1cncc(OC[C@H]2C[C@@H](c3cc(Nc4nccn5nccc45)n[nH]3)C2)c1] | A | |
| 537 | 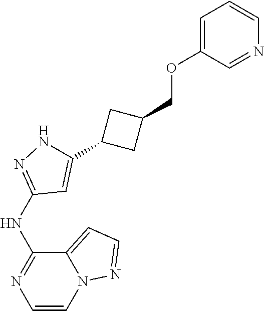 [SMILES: c1cncc(OC[C@H]2C[C@H](c3cc(Nc4nccn5nccc45)n[nH]3)C2)c1] | D | |
| 538 | 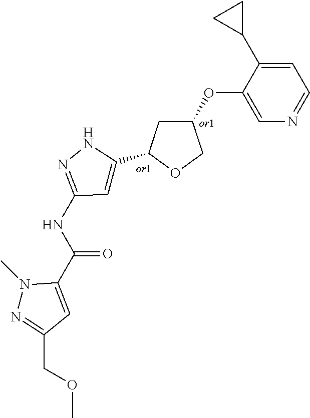 [SMILES: COCc1cc(C(=O)Nc2cc([C@@H]3C[C@H](Oc4cnccc4C4CC4)CO3)[nH]n2)n(C)n1] | D | |
| 539 | 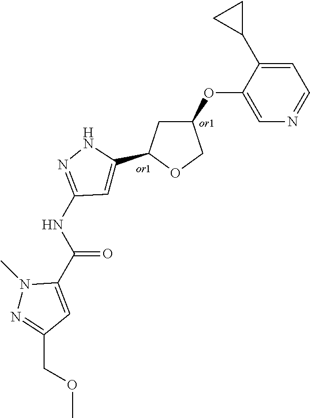 [SMILES: COCc1cc(C(=O)Nc2cc([C@H]3C[C@@H](Oc4cnccc4C4CC4)CO3)[nH]n2)n(C)n1] | C | |
| 540 | 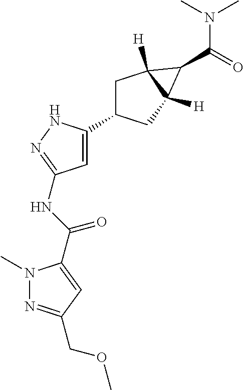 | D | |
| 541 | 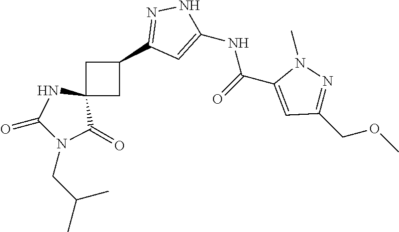 [SMILES: COCc1cc(C(=O)Nc2cc([C@H]3C[C@]4(C3)NC(=O)N(CC(C)C)C4=O)n[nH]2)n(C)n1] | D | |
| 542 | 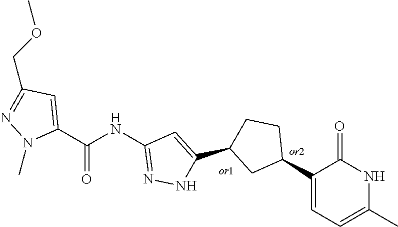 [SMILES: COCc1cc(C(=O)Nc2cc([C@H]3CC[C@@H](c4ccc(C)[nH]c4=O)C3)[nH]n2)n(C)n1] | C | |
| 543 | 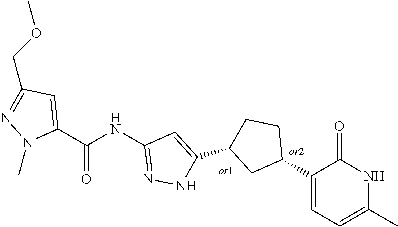 [SMILES: COCc1cc(C(=O)Nc2cc([C@@H]3CC[C@H](c4ccc(C)[nH]c4=O)C3)[nH]n2)n(C)n1] | D | |
| 544 | 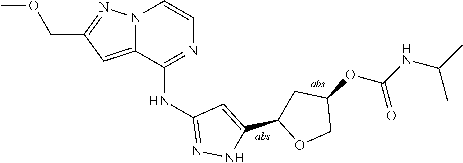 [SMILES: COCc1cc2c(Nc3cc([C@H]4C[C@@H](OC(=O)NC(C)C)CO4)[nH]n3)nccn2n1] | A | |
| 545 | 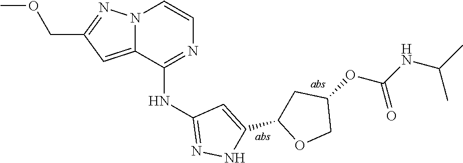 [SMILES: COCc1cc2c(Nc3cc([C@@H]4C[C@H](OC(=O)NC(C)C)CO4)[nH]n3)nccn2n1] | A | |
| 546 | 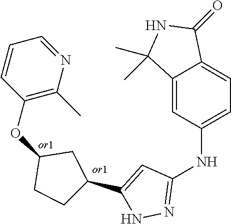 [SMILES: Cc1ncccc1O[C@@H]1CC[C@H](c2cc(Nc3ccc4c(c3)C(C)(C)NC4=O)n[nH]2)C1] | A | |
| 547 | 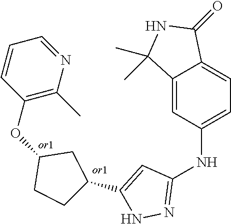 [SMILES: Cc1ncccc1O[C@H]1CC[C@@H](c2cc(Nc3ccc4c(c3)C(C)(C)NC4=O)n[nH]2)C1] | A | |
| 548 | 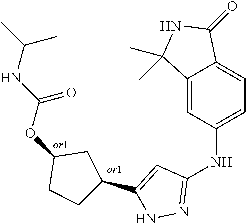 [SMILES: CC(C)NC(=O)O[C@@H]1CC[C@H](c2cc(Nc3ccc4c(c3)C(C)(C)NC4=O)n[nH]2)C1] | A | |
| 549 | 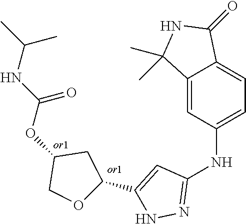 [SMILES: CC(C)NC(=O)O[C@H]1CO[C@@H](c2cc(Nc3ccc4c(c3)C(C)(C)NC4=O)n[nH]2)C1] | A | |
| 550 | 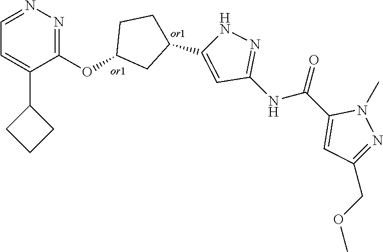 [SMILES: COCc1cc(C(=O)Nc2cc([C@H]3CC[C@@H](Oc4nnccc4C4CCC4)C3)[nH]n2)n(C)n1] | A | |
| 551 | 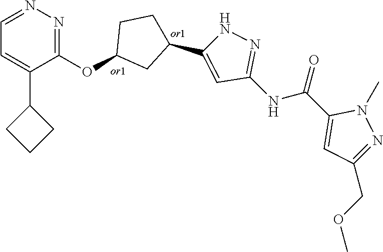 [SMILES: COCc1cc(C(=O)Nc2cc([C@@H]3CC[C@H](Oc4nnccc4C4CCC4)C3)[nH]n2)n(C)n1] | A | |
| 552 | 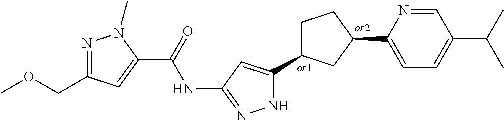 [SMILES: COCc1cc(C(=O)Nc2cc([C@H]3CC[C@@H](c4ccc(C(C)C)cn4)C3)[nH]n2)n(C)n1] | D | |
| 553 | 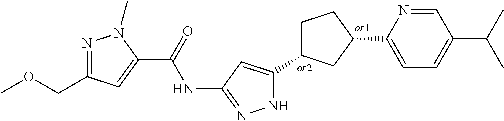 [SMILES: COCc1cc(C(=O)Nc2cc([C@@H]3CC[C@H](c4ccc(C(C)C)cn4)C3)[nH]n2)n(C)n1] | D | |
| 554 | 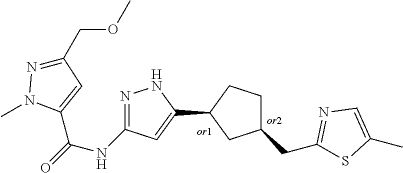 | D | |
| 555 | 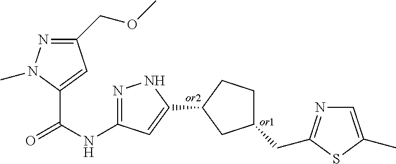 | C | |
| 556 | 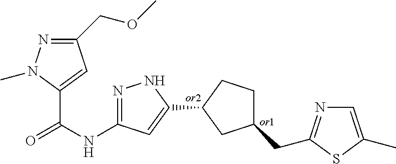 | D | |
| 557 | 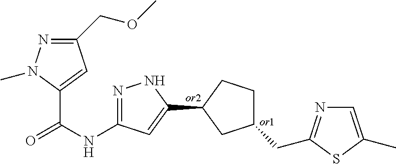 | D | |
| 558 | 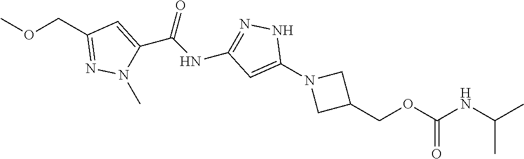 [SMILES: COCc1cc(C(=O)Nc2cc(N3CC(COC(=O)NC(C)C)C3)[nH]n2)n(C)n1] | D | |
| 559 | 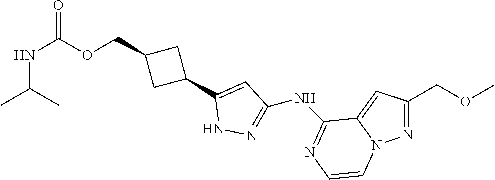 [SMILES: COCc1cc2c(Nc3cc([C@H]4C[C@@H](COC(=O)NC(C)C)C4)[nH]n3)nccn2n1] | | |
| 560 | 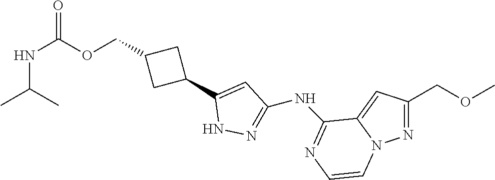 | B | |
| 561 | 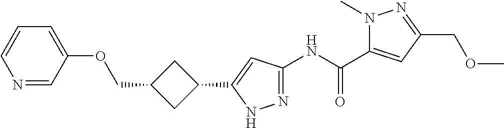 [SMILES: COCc1cc(C(=O)Nc2cc([C@H]3C[C@@H](COc4cccnc4)C3)[nH]n2)n(C)n1] | D | |
| 562 | 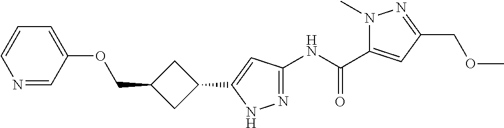 [SMILES: COCc1cc(C(=O)Nc2cc([C@H]3C[C@H](COc4cccnc4)C3)[nH]n2)n(C)n1] | D | |
| 563 | 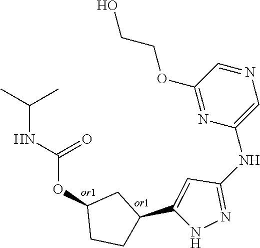 [SMILES: CC(C)NC(=O)O[C@@H]1CC[C@H](c2cc(Nc3cncc(OCCO)n3)n[nH]2)C1] | A | |
| 564 | 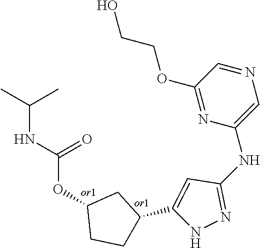 [SMILES: CC(C)NC(=O)O[C@H]1CC[C@@H](c2cc(Nc3cncc(OCCO)n3)n[nH]2)C1] | A | |
| 565 | 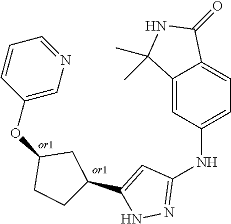 [SMILES: CC1(C)NC(=O)c2ccc(Nc3cc([C@H]4CC[C@@H](Oc5cccnc5)C4)[nH]n3)cc21] | A | |
| 566 | 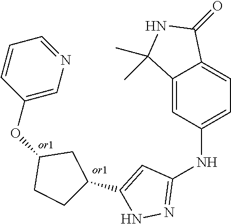 [SMILES: CC1(C)NC(=O)c2ccc(Nc3cc([C@@H]4CC[C@H](Oc5cccnc5)C4)[nH]n3)cc21] | | |
| 567 | 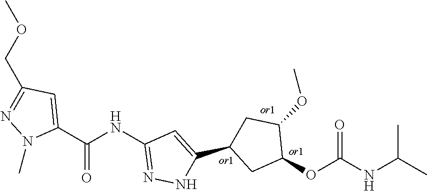 [SMILES: COCc1cc(C(=O)Nc2cc([C@H]3C[C@H](OC)[C@@H](OC(=O)NC(C)C)C3)[nH]n2)n(C)n1] | D | |
| 568 | 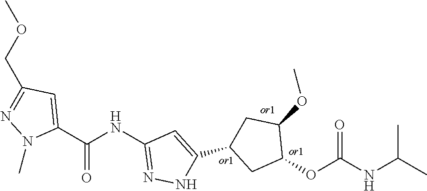 [SMILES: COCc1cc(C(=O)Nc2cc([C@@H]3C[C@@H](OC)[C@H](OC(=O)NC(C)C)C3)[nH]n2)n(C)n1] | D | |
| 569 | 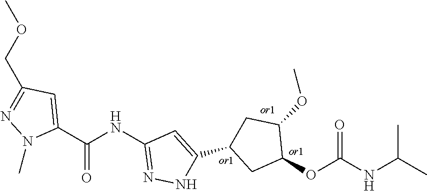 [SMILES: COCc1cc(C(=O)Nc2cc([C@@H]3C[C@H](OC)[C@@H](OC(=O)NC(C)C)C3)[nH]n2)n(C)n1] | D | |
| 570 | 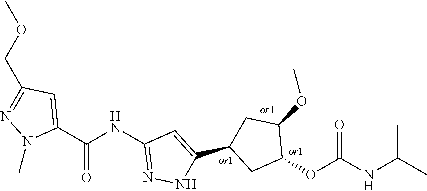 [SMILES: COCc1cc(C(=O)Nc2cc([C@H]3C[C@@H](OC)[C@H](OC(=O)NC(C)C)C3)[nH]n2)n(C)n1] | D | |
| 571 | 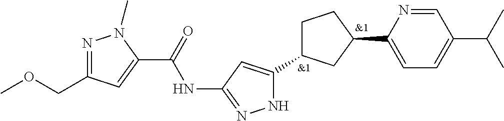 [SMILES: COCc1cc(C(=O)Nc2cc([C@@H]3CC[C@@H](c4ccc(C(C)C)cn4)C3)[nH]n2)n(C)n1] | D | |
| 572 | 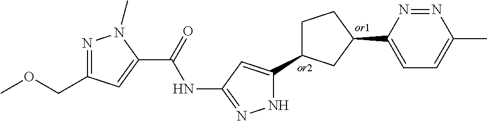 | D | |
| 573 | 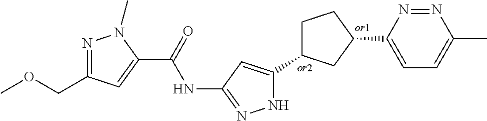 | D | |
| 574 | 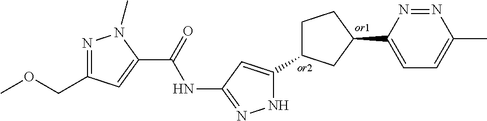 | D | |
| 575 | 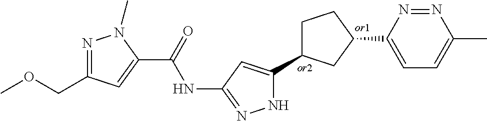 | D | |
| 576 | 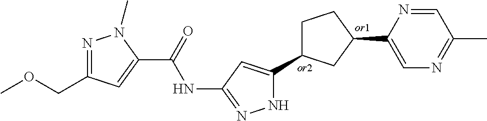 | D | |
| 577 | 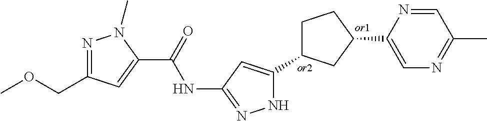 | D | |
| 578 | 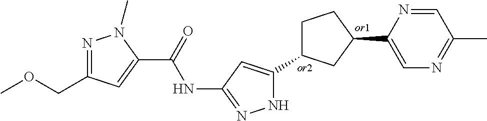 | D | |
| 579 | 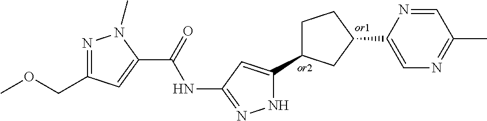 | D | |
| 580 | 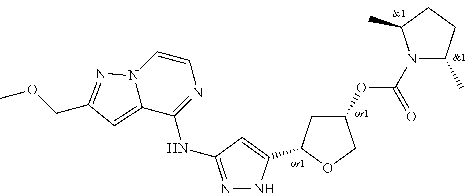 | A | |
| 581 | 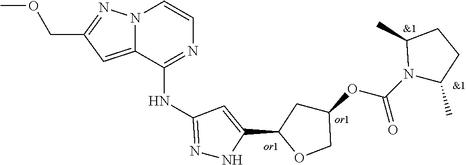 [SMILES: COCc1cc2c(Nc3cc([C@H]4C[C@@H](OC(=O)N5[C@@H](C)CC[C@@H]5C)CO4)[nH]n3)nccn2n1] | A | |
| 582 | 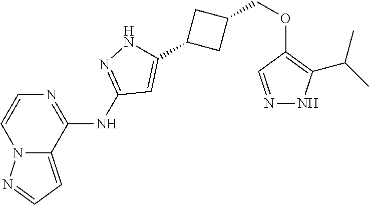 [SMILES: CC(C)c1[nH]ncc1OC[C@H]1C[C@@H](c2cc(Nc3nccn4nccc34)n[nH]2)C1] | A | |
| 583 | 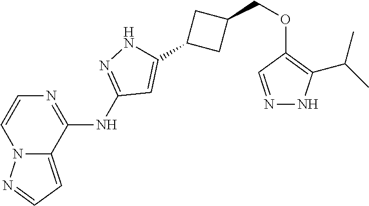 [SMILES: CC(C)c1[nH]ncc1OC[C@H]1C[C@H](c2cc(Nc3nccn4nccc34)n[nH]2)C1] | D | |
| 584 | 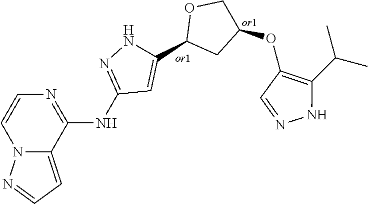 [SMILES: CC(C)c1[nH]ncc1O[C@@H]1CO[C@H](c2cc(Nc3nccn4nccc34)n[nH]2)C1] | D | |
| 585 | 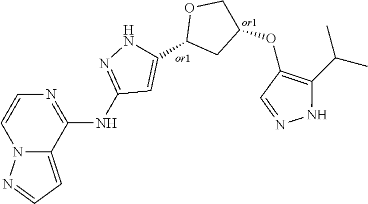 [SMILES: CC(C)c1[nH]ncc1O[C@H]1CO[C@@H](c2cc(Nc3nccn4nccc34)n[nH]2)C1] | D | |
| 586 | 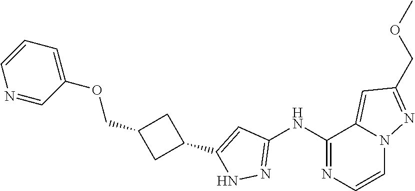 [SMILES: COCc1cc2c(Nc3cc([C@H]4C[C@@H](COc5cccnc5)C4)[nH]n3)nccn2n1] | C | |
| 587 | 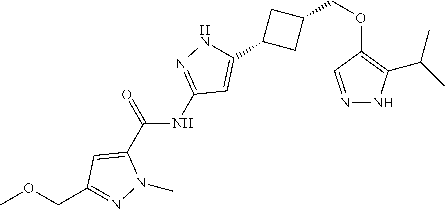 [SMILES: COCc1cc(C(=O)Nc2cc([C@H]3C[C@@H](COc4cn[nH]c4C(C)C)C3)[nH]n2)n(C)n1] | B | |
| 588 | 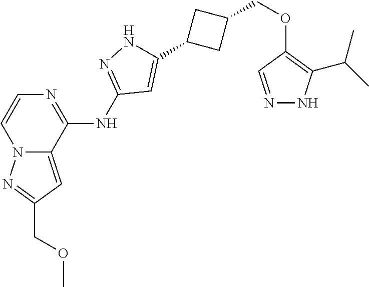 [SMILES: COCc1cc2c(Nc3cc([C@H]4C[C@@H](COc5cn[nH]c5C(C)C)C4)[nH]n3)nccn2n1] | A | |
| 589 | 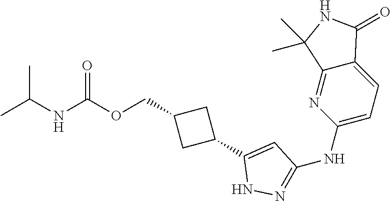 [SMILES: CC(C)NC(=O)OC[C@H]1C[C@@H](c2cc(Nc3ccc4c(n3)C(C)(C)NC4=O)n[nH]2)C1] | A | |
| 590 | 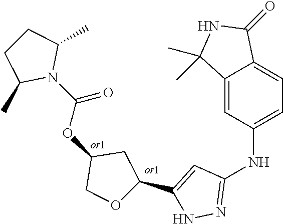 [SMILES: C[C@H]1CC[C@H](C)N1C(=O)O[C@@H]1CO[C@H](c2cc(Nc3ccc4c(c3)C(C)(C)NC4=O)n[nH]2)C1] | A | |
| 591 | 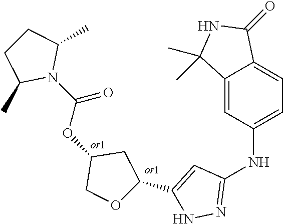 [SMILES: C[C@H]1CC[C@H](C)N1C(=O)O[C@H]1CO[C@@H](c2cc(Nc3ccc4c(c3)C(C)(C)NC4=O)n[nH]2)C1] | A | |
| 592 | 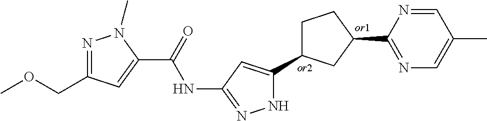 | D | |
| 593 | 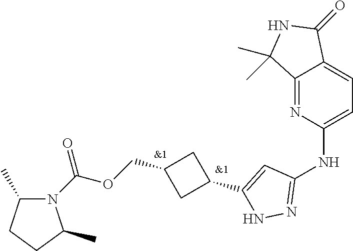 [SMILES: C[C@H]1CC[C@H](C)N1C(=O)OC[C@H]1C[C@@H](c2cc(Nc3ccc4c(n3)C(C)(C)NC4=O)n[nH]2)C1] | A | |
| 594 | 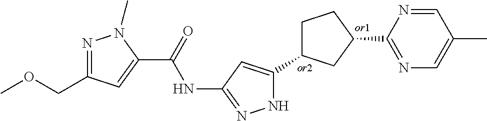 | D | |
| 595 | 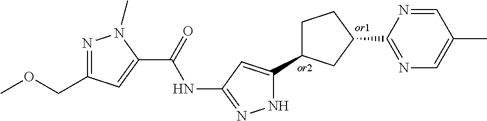 | D | |
| 596 | 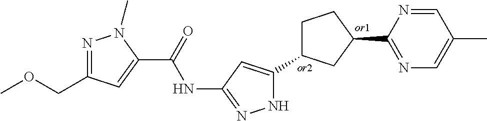 | D | |
| 597 | 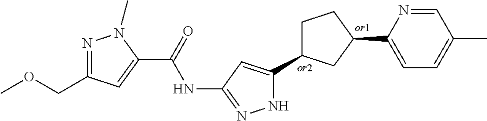 | D | |
| 598 | 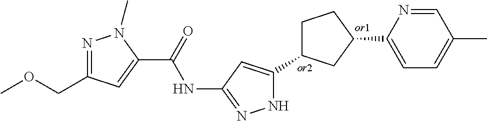 | D | |
| 599 | 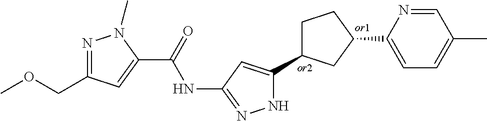 | D | |
| 600 | 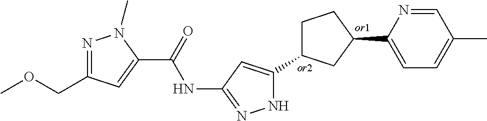 | D | |
| 601 | 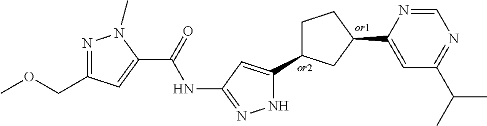 [SMILES: COCc1cc(C(=O)Nc2cc([C@H]3CC[C@@H](c4cc(C(C)C)ncn4)C3)[nH]n2)n(C)n1] | D | |
| 602 | 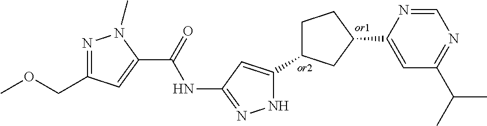 [SMILES: COCc1cc(C(=O)Nc2cc([C@@H]3CC[C@H](c4cc(C(C)C)ncn4)C3)[nH]n2)n(C)n1] | D | |
| 603 | 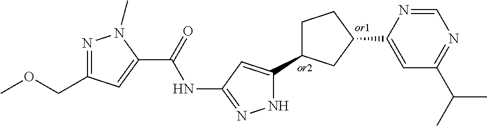 [SMILES: COCc1cc(C(=O)Nc2cc([C@H]3CC[C@H](c4cc(C(C)C)ncn4)C3)[nH]n2)n(C)n1] | D | |
| 604 | 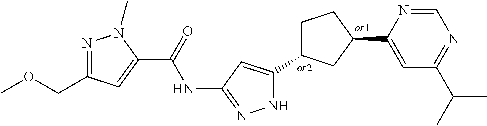 [SMILES: COCc1cc(C(=O)Nc2cc([C@@H]3CC[C@@H](c4cc(C(C)C)ncn4)C3)[nH]n2)n(C)n1] | D | |
| 605 | 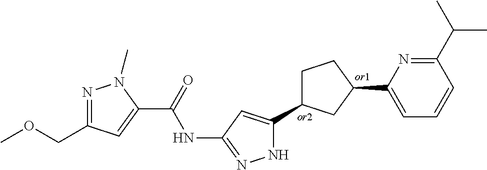 [SMILES: COCc1cc(C(=O)Nc2cc([C@H]3CC[C@@H](c4cccc(C(C)C)n4)C3)[nH]n2)n(C)n1] | D | |
| 606 | 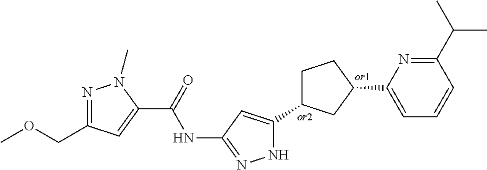 [SMILES: COCc1cc(C(=O)Nc2cc([C@@H]3CC[C@H](c4cccc(C(C)C)n4)C3)[nH]n2)n(C)n1] | D | |
| 607 | 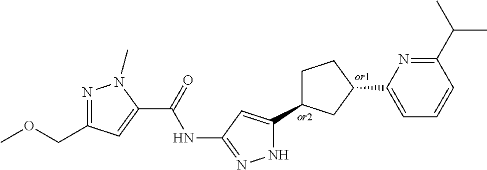 [SMILES: COCc1cc(C(=O)Nc2cc([C@H]3CC[C@H](c4cccc(C(C)C)n4)C3)[nH]n2)n(C)n1] | D | |
| 608 | 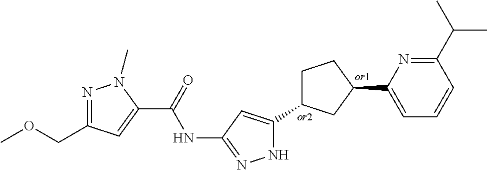 [SMILES: COCc1cc(C(=O)Nc2cc([C@@H]3CC[C@@H](c4cccc(C(C)C)n4)C3)[nH]n2)n(C)n1] | D | |
| 609 | 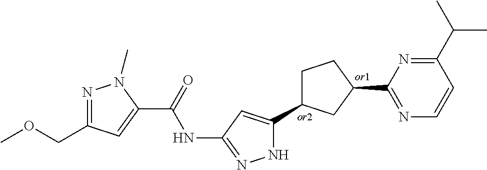 [SMILES: COCc1cc(C(=O)Nc2cc([C@H]3CC[C@@H](c4nccc(C(C)C)n4)C3)[nH]n2)n(C)n1] | A | |
| 610 | 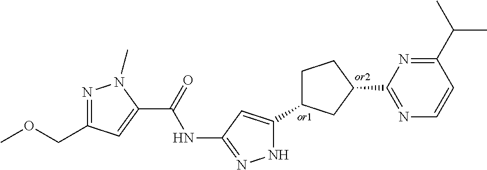 [SMILES: COCc1cc(C(=O)Nc2cc([C@@H]3CC[C@H](c4nccc(C(C)C)n4)C3)[nH]n2)n(C)n1] | A | |
| 611 | 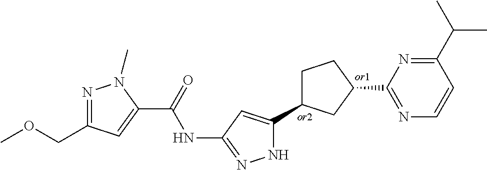 [SMILES: COCc1cc(C(=O)Nc2cc([C@H]3CC[C@H](c4nccc(C(C)C)n4)C3)[nH]n2)n(C)n1] | D | |
| 612 | 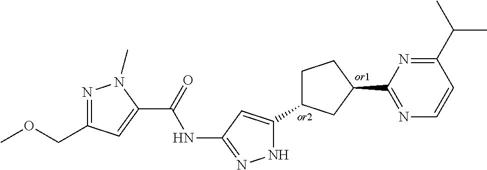 [SMILES: COCc1cc(C(=O)Nc2cc([C@@H]3CC[C@@H](c4nccc(C(C)C)n4)C3)[nH]n2)n(C)n1] | D | |
| 613 | 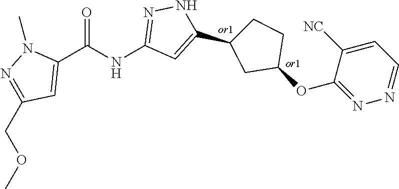 [SMILES: COCc1cc(C(=O)Nc2cc([C@H]3CC[C@@H](Oc4nnccc4C#N)C3)[nH]n2)n(C)n1] | | |
| 614 | 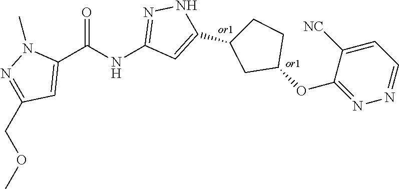 [SMILES: COCc1cc(C(=O)Nc2cc([C@@H]3CC[C@H](Oc4nnccc4C#N)C3)[nH]n2)n(C)n1] | | |
| 615 | 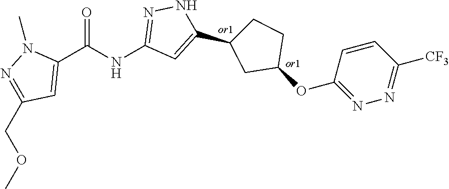 [SMILES: COCc1cc(C(=O)Nc2cc([C@H]3CC[C@@H](Oc4ccc(C(F)(F)F)nn4)C3)[nH]n2)n(C)n1] | | |
| 616 | 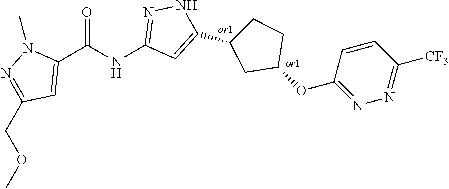 [SMILES: COCc1cc(C(=O)Nc2cc([C@@H]3CC[C@H](Oc4ccc(C(F)(F)F)nn4)C3)[nH]n2)n(C)n1] | | |
| 617 | 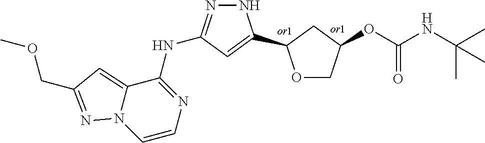 [SMILES: COCc1cc2c(Nc3cc([C@H]4C[C@@H](OC(=O)NC(C)(C)C)CO4)[nH]n3)nccn2n1] | A | |
| 618 | 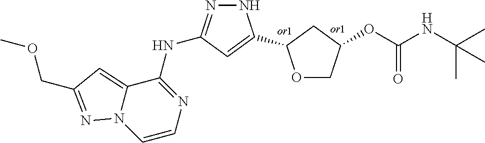 [SMILES: COCc1cc2c(Nc3cc([C@@H]4C[C@H](OC(=O)NC(C)(C)C)CO4)[nH]n3)nccn2n1] | A | |
| 619 | 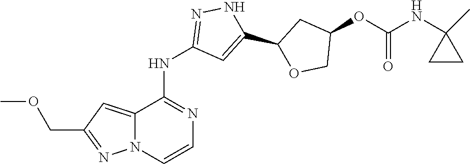 | A | |
| 620 | 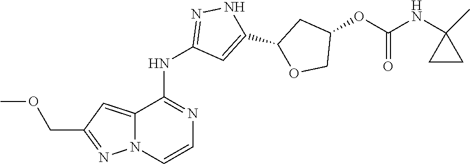 | A | |
| 621 | 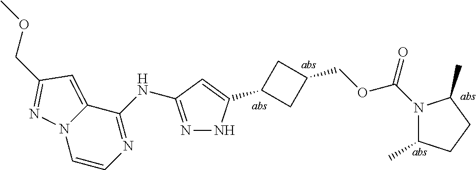 [SMILES: COCc1cc2c(Nc3cc([C@H]4C[C@@H](COC(=O)N5[C@@H](C)CC[C@@H]5C)C4)[nH]n3)nccn2n1] | A | |
| 622 | 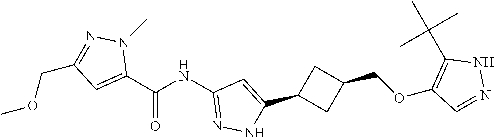 [SMILES: COCc1cc(C(=O)Nc2cc([C@H]3C[C@@H](COc4cn[nH]c4C(C)(C)C)C3)[nH]n2)n(C)n1] | A | |
| 623 | 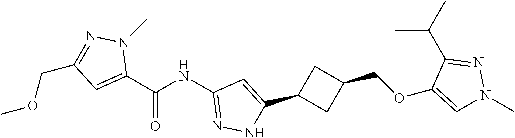 | B | |
| 624 | 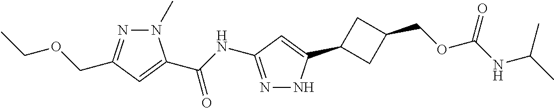 [SMILES: CCOCc1cc(C(=O)Nc2cc([C@H]3C[C@@H](COC(=O)NC(C)C)C3)[nH]n2)n(C)n1] | A | |
| 625 | 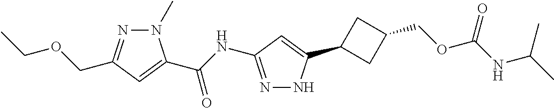 [SMILES: CCOCc1cc(C(=O)Nc2cc([C@H]3C[C@H](COC(=O)NC(C)C)C3)[nH]n2)n(C)n1] | D | |
| 626 | 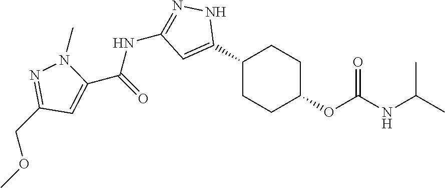 [SMILES: COCc1cc(C(=O)Nc2cc([C@H]3CC[C@@H](OC(=O)NC(C)C)CC3)[nH]n2)n(C)n1] | C | |
| 627 | 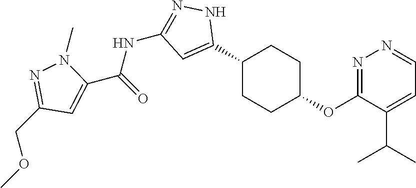 [SMILES: COCc1cc(C(=O)Nc2cc([C@H]3CC[C@@H](Oc4nnccc4C(C)C)CC3)[nH]n2)n(C)n1] | A | |
| 628 | 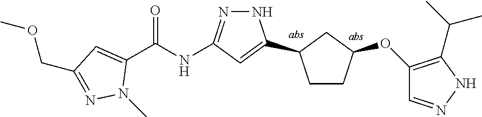 [SMILES: COCc1cc(C(=O)Nc2cc([C@@H]3CC[C@H](Oc4cn[nH]c4C(C)C)C3)[nH]n2)n(C)n1] | C | |
| 629 | 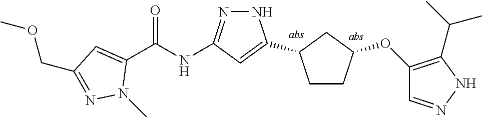 [SMILES: COCc1cc(C(=O)Nc2cc([C@H]3CC[C@@H](Oc4cn[nH]c4C(C)C)C3)[nH]n2)n(C)n1] | B | |
| 630 | 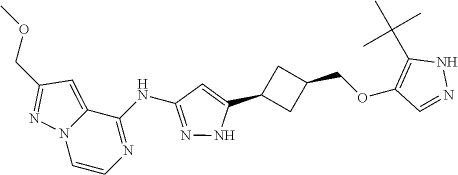 [SMILES: COCc1cc2c(Nc3cc([C@H]4C[C@@H](COc5cn[nH]c5C(C)(C)C)C4)[nH]n3)nccn2n1] | A | |
| 631 | 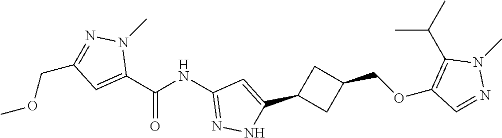 | B | |
| 632 | 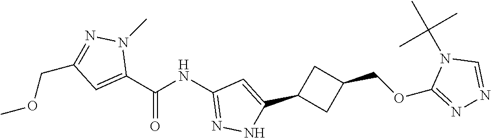 [SMILES: COCc1cc(C(=O)Nc2cc([C@H]3C[C@@H](COc4nncn4C(C)(C)C)C3)[nH]n2)n(C)n1] | A | |
| 633 | 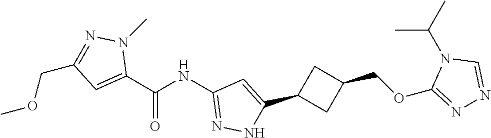 [SMILES: COCc1cc(C(=O)Nc2cc([C@H]3C[C@@H](COc4nncn4C(C)C)C3)[nH]n2)n(C)n1] | A | |
| 634 | 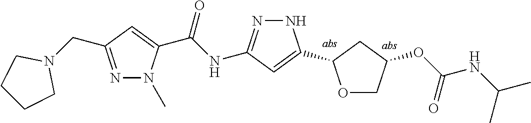 [SMILES: CC(C)NC(=O)O[C@@H]1CO[C@H](c2cc(NC(=O)c3cc(CN4CCCC4)nn3C)n[nH]2)C1] | B | |
| 635 | 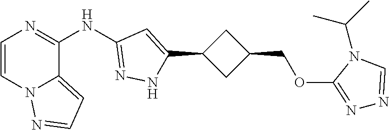 [SMILES: CC(C)n1cnnc1OC[C@H]1C[C@@H](c2cc(Nc3nccn4nccc34)n[nH]2)C1] | A | |
| 636 | 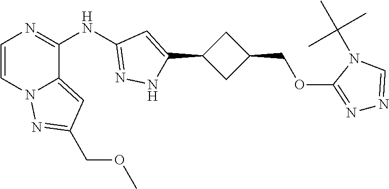 [SMILES: COCc1cc2c(Nc3cc([C@H]4C[C@@H](COc5nncn5C(C)(C)C)C4)[nH]n3)nccn2n1] | A | |
| 637 | 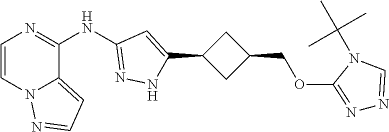 [SMILES: CC(C)(C)n1cnnc1OC[C@H]1C[C@@H](c2cc(Nc3nccn4nccc34)n[nH]2)C1] | A | |
| 638 | 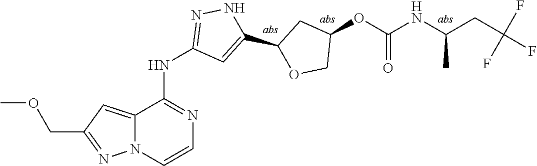 [SMILES: COCc1cc2c(Nc3cc([C@H]4C[C@@H](OC(=O)N[C@H](C)CC(F)(F)F)CO4)[nH]n3)nccn2n1] | A | |
| 639 | 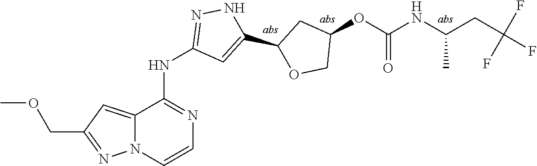 [SMILES: COCc1cc2c(Nc3cc([C@H]4C[C@@H](OC(=O)N[C@@H](C)CC(F)(F)F)CO4)[nH]n3)nccn2n1] | A | |
| 640 | 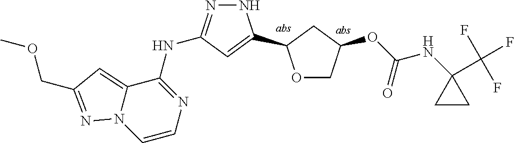 [SMILES: COCc1cc2c(Nc3cc([C@H]4C[C@@H](OC(=O)NC5(C(F)(F)F)CC5)CO4)[nH]n3)nccn2n1] | A | |
| 641 | 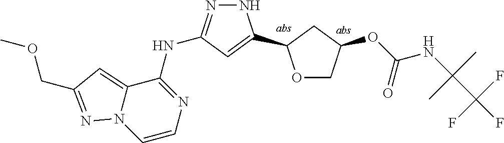 [SMILES: COCc1cc2c(Nc3cc([C@H]4C[C@@H](OC(=O)NC(C)(C)C(F)(F)F)CO4)[nH]n3)nccn2n1] | A | |
| 642 | 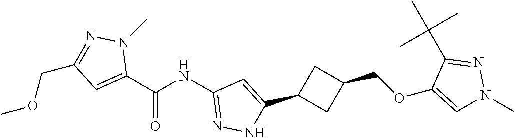 | B | |
| 643 | 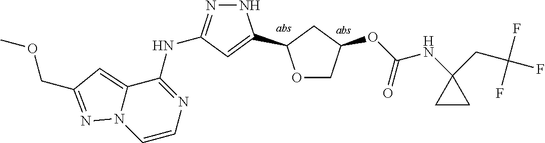 [SMILES: COCc1cc2c(Nc3cc([C@H]4C[C@@H](OC(=O)NC5(CC(F)(F)F)CC5)CO4)[nH]n3)nccn2n1] | A | |
| 644 | 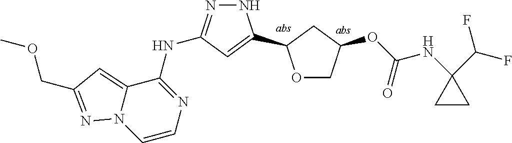 [SMILES: COCc1cc2c(Nc3cc([C@H]4C[C@@H](OC(=O)NC5(C(F)F)CC5)CO4)[nH]n3)nccn2n1] | A | |
| 645 | 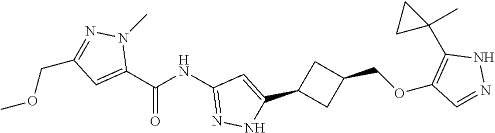 [SMILES: COCc1cc(C(=O)Nc2cc([C@H]3C[C@@H](COc4cn[nH]c4C4(C)CC4)C3)[nH]n2)n(C)n1] | C | |
| 646 | 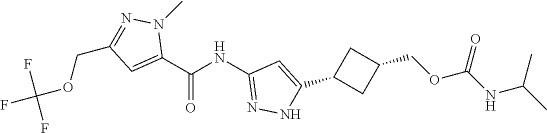 [SMILES: CC(C)NC(=O)OC[C@H]1C[C@@H](c2cc(NC(=O)c3cc(COC(F)(F)F)nn3C)n[nH]2)C1] | A | |
| 647 | 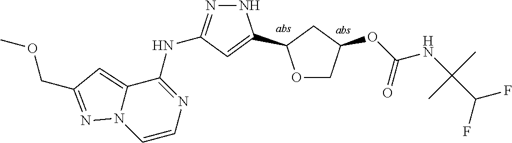 [SMILES: COCc1cc2c(Nc3cc([C@H]4C[C@@H](OC(=O)NC(C)(C)C(F)F)CO4)[nH]n3)nccn2n1] | A | |
| 648 | 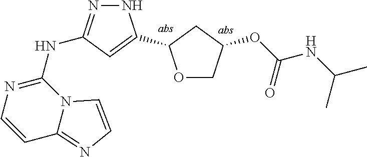 [SMILES: CC(C)NC(=O)O[C@@H]1CO[C@H](c2cc(Nc3nccc4nccn34)n[nH]2)C1] | A | |
| 649 | 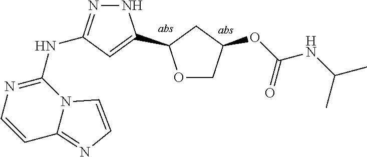 [SMILES: CC(C)NC(=O)O[C@H]1CO[C@@H](c2cc(Nc3nccc4nccn34)n[nH]2)C1] | A | |
| 650 | 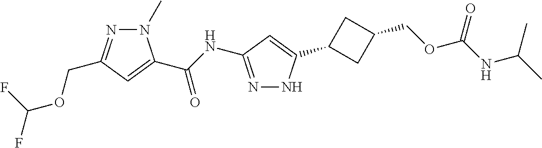 [SMILES: CC(C)NC(=O)OC[C@H]1C[C@@H](c2cc(NC(=O)c3cc(COC(F)F)nn3C)n[nH]2)C1] | A | |
| 651 | 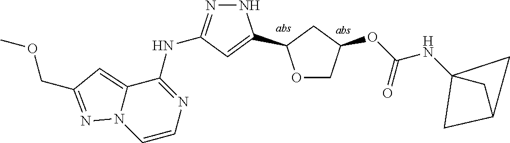 [SMILES: COCc1cc2c(Nc3cc([C@H]4C[C@@H](OC(=O)NC56CC(C5)C6)CO4)[nH]n3)nccn2n1] | A | |
| 652 | 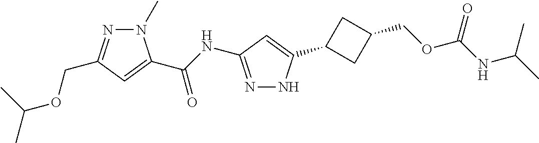 [SMILES: CC(C)NC(=O)OC[C@H]1C[C@@H](c2cc(NC(=O)c3cc(COC(C)C)nn3C)n[nH]2)C1] | A | |
| 653 | 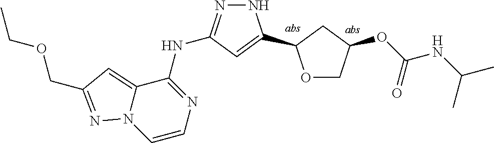 [SMILES: CCOCc1cc2c(Nc3cc([C@H]4C[C@@H](OC(=O)NC(C)C)CO4)[nH]n3)nccn2n1] | A | |
| 654 | 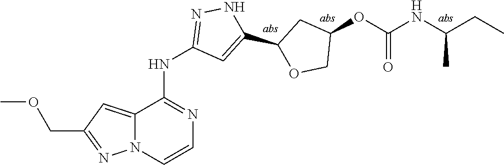 [SMILES: CC[C@@H](C)NC(=O)O[C@H]1CO[C@@H](c2cc(Nc3nccn4nc(COC)cc34)n[nH]2)C1] | A | |
| 655 | 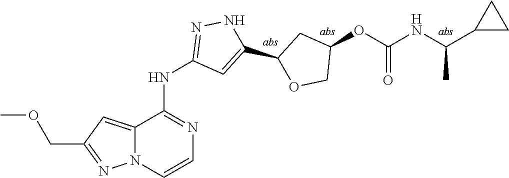 [SMILES: COCc1cc2c(Nc3cc([C@H]4C[C@@H](OC(=O)N[C@H](C)C5CC5)CO4)[nH]n3)nccn2n1] | A | |
| 656 | 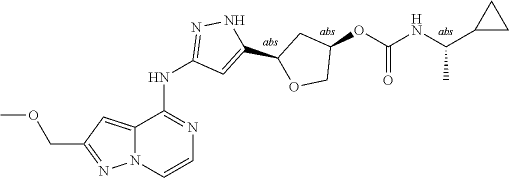 [SMILES: COCc1cc2c(Nc3cc([C@H]4C[C@@H](OC(=O)N[C@@H](C)C5CC5)CO4)[nH]n3)nccn2n1] | A | |
| 657 | 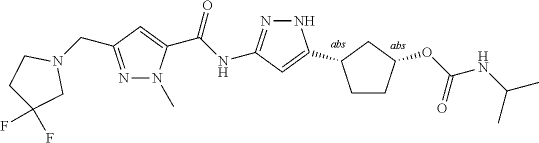 [SMILES: CC(C)NC(=O)O[C@@H]1CC[C@H](c2cc(NC(=O)c3cc(CN4CCC(F)(F)C4)nn3C)n[nH]2)C1] | A | |
| 658 | 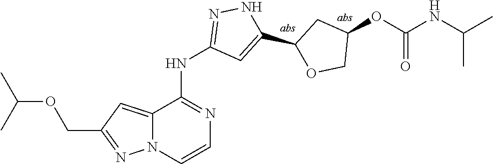 [SMILES: CC(C)NC(=O)O[C@H]1CO[C@@H](c2cc(Nc3nccn4nc(COC(C)C)cc34)n[nH]2)C1] | A | |
| 659 | 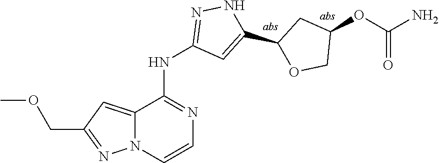 [SMILES: COCc1cc2c(Nc3cc([C@H]4C[C@@H](OC(N)=O)CO4)[nH]n3)nccn2n1] | D | |
| 660 | 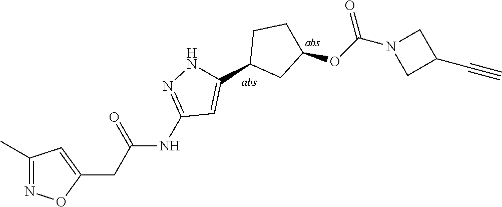 [SMILES: C#CC1CN(C(=O)O[C@@H]2CC[C@H](c3cc(NC(=O)Cc4cc(C)no4)n[nH]3)C2)C1] | B | |
| 661 | 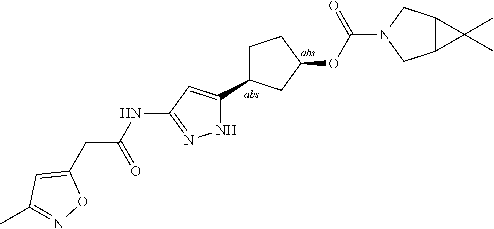 | A | |
| 662 | 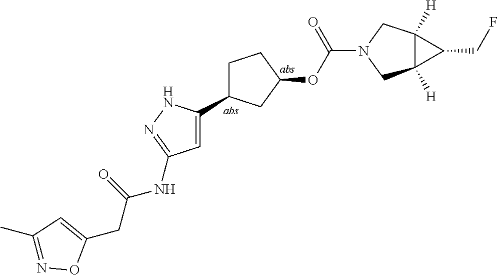 [SMILES: Cc1cc(CC(=O)Nc2cc([C@H]3CC[C@@H](OC(=O)N4C[C@@H]5[C@@H](CF)[C@@H]5C4)C3)[nH]n2)on1] | A | |
| 663 | 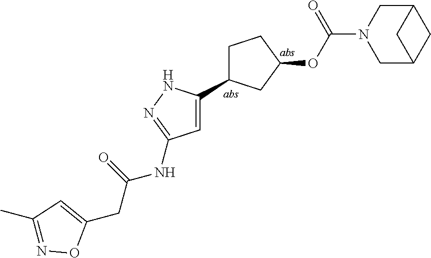 [SMILES: Cc1cc(CC(=O)Nc2cc([C@H]3CC[C@@H](OC(=O)N4CC5CC(C5)C4)C3)[nH]n2)on1] | A | |
| 664 | 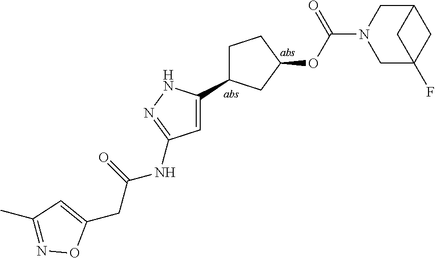 [SMILES: Cc1cc(CC(=O)Nc2cc([C@H]3CC[C@@H](OC(=O)N4CC5CC(F)(C5)C4)C3)[nH]n2)on1] | B | |
| 665 | 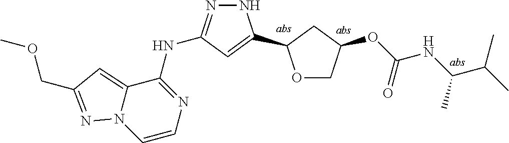 [SMILES: COCc1cc2c(Nc3cc([C@H]4C[C@@H](OC(=O)N[C@@H](C)C(C)C)CO4)[nH]n3)nccn2n1] | A | |
| 666 | 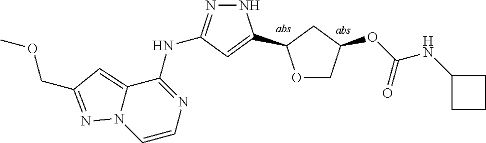 [SMILES: COCc1cc2c(Nc3cc([C@H]4C[C@@H](OC(=O)NC5CCC5)CO4)[nH]n3)nccn2n1] | A | |
| 667 | 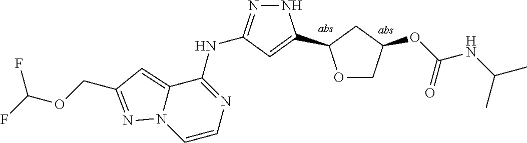 [SMILES: CC(C)NC(=O)O[C@H]1CO[C@@H](c2cc(Nc3nccn4nc(COC(F)F)cc34)n[nH]2)C1] | A | |
| 668 | 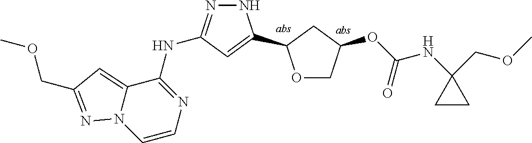 | A | |
| 669 | 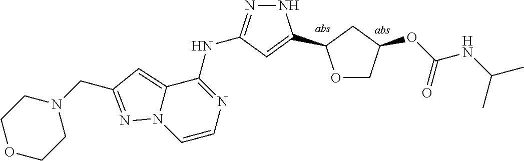 [SMILES: CC(C)NC(=O)O[C@H]1CO[C@@H](c2cc(Nc3nccn4nc(CN5CCOCC5)cc34)n[nH]2)C1] | A | |
| 670 | 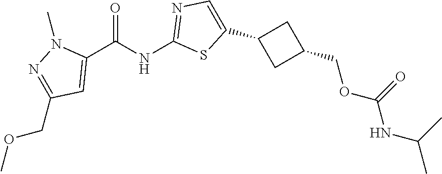 [SMILES: COCc1cc(C(=O)Nc2ncc([C@H]3C[C@@H](COC(=O)NC(C)C)C3)s2)n(C)n1] | A | |
| 671 | 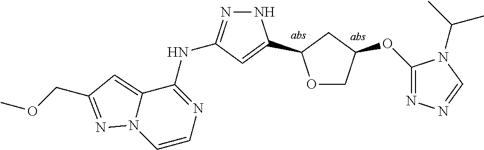 [SMILES: COCc1cc2c(Nc3cc([C@H]4C[C@@H](Oc5nncn5C(C)C)CO4)[nH]n3)nccn2n1] | B | |
| 672 | 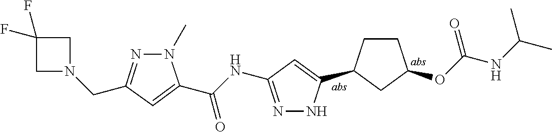 [SMILES: CC(C)NC(=O)O[C@@H]1CC[C@H](c2cc(NC(=O)c3cc(CN4CC(F)(F)C4)nn3C)n[nH]2)C1] | A | |
| 673 | 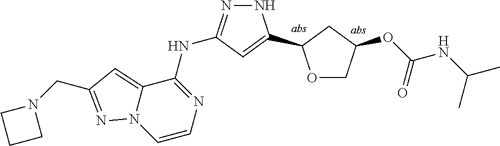 [SMILES: CC(C)NC(=O)O[C@H]1CO[C@@H](c2cc(Nc3nccn4nc(CN5CCC5)cc34)n[nH]2)C1] | A | |
| 674 | 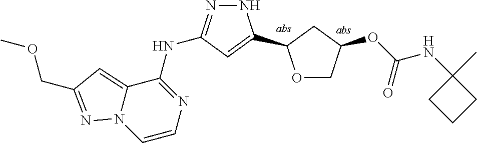 [SMILES: COCc1cc2c(Nc3cc([C@H]4C[C@@H](OC(=O)NC5(C)CCC5)CO4)[nH]n3)nccn2n1] | A | |
| 675 | 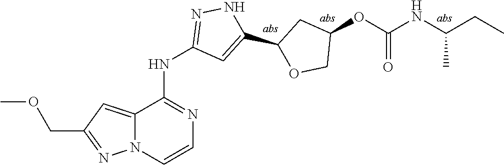 [SMILES: CC[C@H](C)NC(=O)O[C@H]1CO[C@@H](c2cc(Nc3nccn4nc(COC)cc34)n[nH]2)C1] | A | |
| 676 | 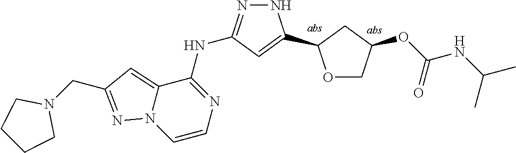 [SMILES: CC(C)NC(=O)O[C@H]1CO[C@@H](c2cc(Nc3nccn4nc(CN5CCCC5)cc34)n[nH]2)C1] | A | |
| 677 | 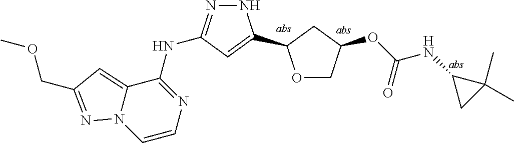 | A | |
| 678 | 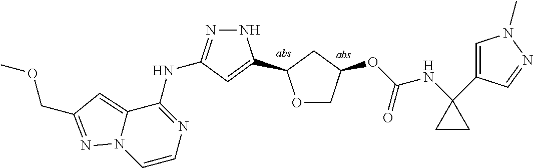 [SMILES: COCc1cc2c(Nc3cc([C@H]4C[C@@H](OC(=O)NC5(c6cnn(C)c6)CC5)CO4)[nH]n3)nccn2n1] | B | |
| 679 | 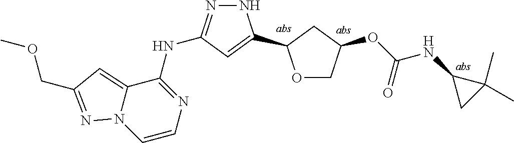 | A | |
| 680 | 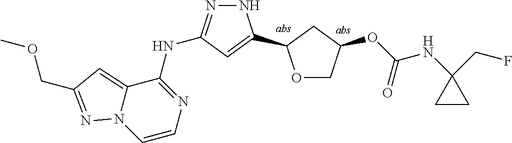 [SMILES: COCc1cc2c(Nc3cc([C@H]4C[C@@H](OC(=O)NC5(CF)CC5)CO4)[nH]n3)nccn2n1] | A | |
| 681 | 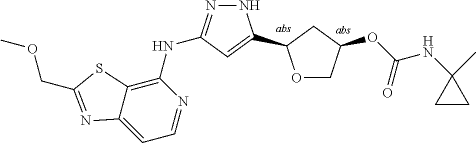 | A | |
| 682 | 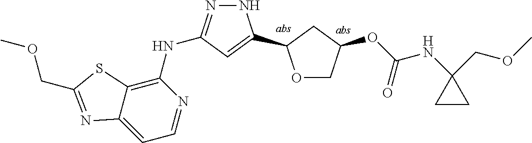 | A | |
| 683 | 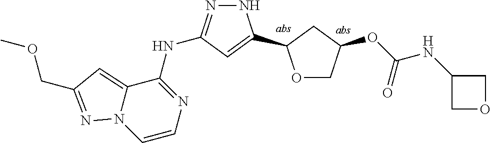 [SMILES: COCc1cc2c(Nc3cc([C@H]4C[C@@H](OC(=O)NC5COC5)CO4)[nH]n3)nccn2n1] | C | |
| 684 | 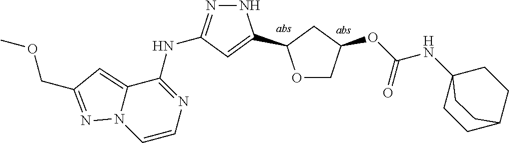 [SMILES: COCc1cc2c(Nc3cc([C@H]4C[C@@H](OC(=O)NC56CCC(CC5)CC6)CO4)[nH]n3)nccn2n1] | A | |
| 685 | 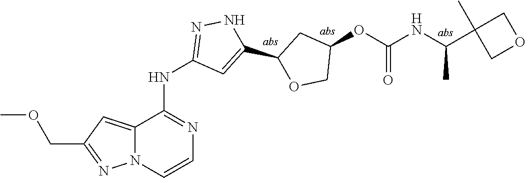 [SMILES: COCc1cc2c(Nc3cc([C@H]4C[C@@H](OC(=O)N[C@H](C)C5(C)COC5)CO4)[nH]n3)nccn2n1] | D | |
| 686 | 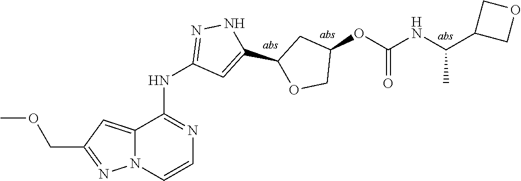 [SMILES: COCc1cc2c(Nc3cc([C@H]4C[C@@H](OC(=O)N[C@@H](C)C5COC5)CO4)[nH]n3)nccn2n1] | B | |
| 687 | 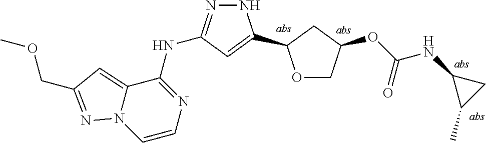 [SMILES: COCc1cc2c(Nc3cc([C@H]4C[C@@H](OC(=O)N[C@H]5C[C@@H]5C)CO4)[nH]n3)nccn2n1] | A | |
| 688 | 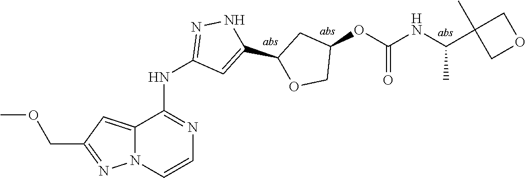 [SMILES: COCc1cc2c(Nc3cc([C@H]4C[C@@H](OC(=O)N[C@@H](C)C5(C)COC5)CO4)[nH]n3)nccn2n1] | B | |
| 689 | 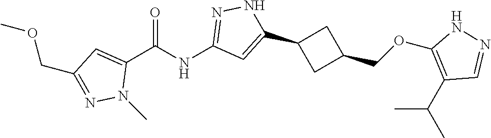 [SMILES: COCc1cc(C(=O)Nc2cc([C@H]3C[C@@H](COc4[nH]ncc4C(C)C)C3)[nH]n2)n(C)n1] | A | |
| 690 | 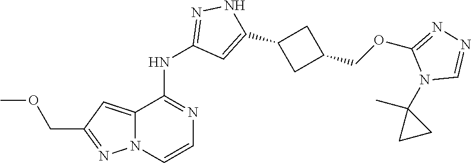 [SMILES: COCc1cc2c(Nc3cc([C@H]4C[C@@H](COc5nncn5C5(C)CC5)C4)[nH]n3)nccn2n1] | A | |
| 691 | 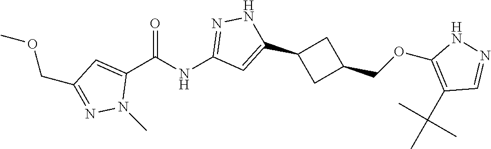 [SMILES: COCc1cc(C(=O)Nc2cc([C@H]3C[C@@H](COc4[nH]ncc4C(C)(C)C)C3)[nH]n2)n(C)n1] | A | |
| 692 | 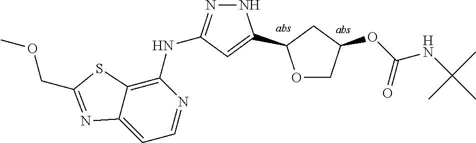 [SMILES: COCc1nc2ccnc(Nc3cc([C@H]4C[C@@H](OC(=O)NC(C)(C)C)CO4)[nH]n3)c2s1] | A | |
| 693 | 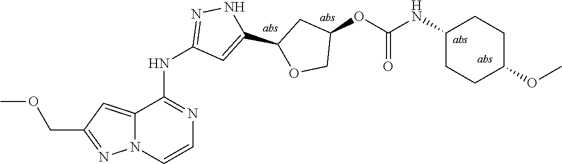 | D | |
| 694 | 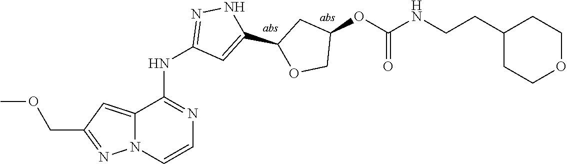 [SMILES: COCc1cc2c(Nc3cc([C@H]4C[C@@H](OC(=O)NCCC5CCOCC5)CO4)[nH]n3)nccn2n1] | D | |
| 695 | 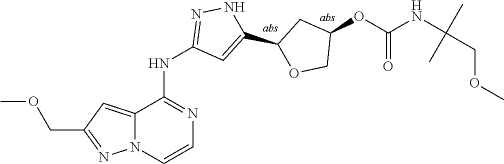 | A | |
| 696 | 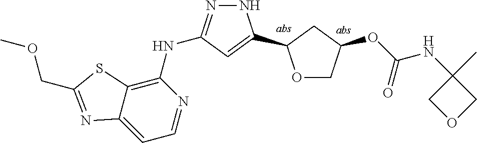 [SMILES: COCc1nc2ccnc(Nc3cc([C@H]4C[C@@H](OC(=O)NC5(C)COC5)CO4)[nH]n3)c2s1] | A | |
| 697 | 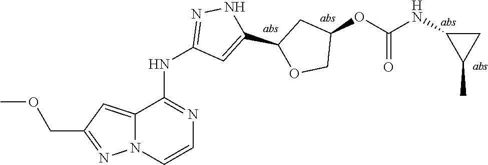 [SMILES: COCc1cc2c(Nc3cc([C@H]4C[C@@H](OC(=O)N[C@@H]5C[C@H]5C)CO4)[nH]n3)nccn2n1] | A | |
| 698 | 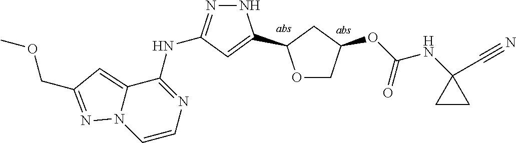 [SMILES: COCc1cc2c(Nc3cc([C@H]4C[C@@H](OC(=O)NC5(C#N)CC5)CO4)[nH]n3)nccn2n1] | A | |
| 699 | 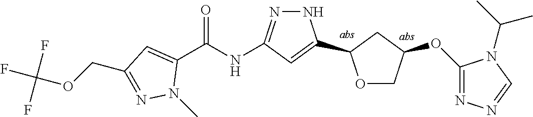 [SMILES: CC(C)n1cnnc1O[C@H]1CO[C@@H](c2cc(NC(=O)c3cc(COC(F)(F)F)nn3C)n[nH]2)C1] | A | |
| 700 | 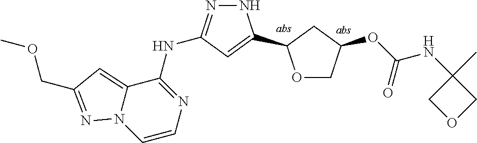 [SMILES: COCc1cc2c(Nc3cc([C@H]4C[C@@H](OC(=O)NC5(C)COC5)CO4)[nH]n3)nccn2n1] | A | |
| 701 | 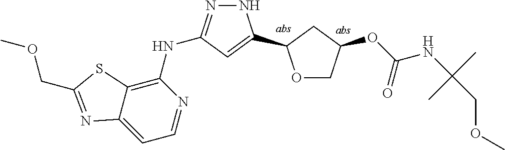 | A | |
| 702 | 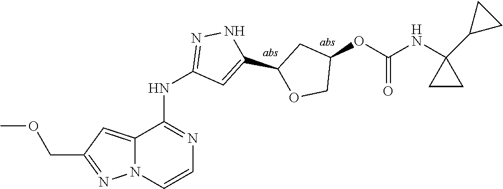 [SMILES: COCc1cc2c(Nc3cc([C@H]4C[C@@H](OC(=O)NC5(C6CC6)CC5)CO4)[nH]n3)nccn2n1] | A | |
| 703 | 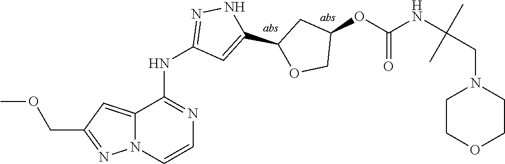 [SMILES: COCc1cc2c(Nc3cc([C@H]4C[C@@H](OC(=O)NC(C)(C)CN5CCOCC5)CO4)[nH]n3)nccn2n1] | C | |
| 704 | 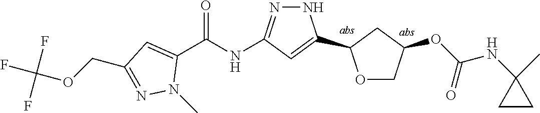 [SMILES: Cn1nc(COC(F)(F)F)cc1C(=O)Nc1cc([C@H]2C[C@@H](OC(=O)NC3(C)CC3)CO2)[nH]n1] | A | |
| 705 | 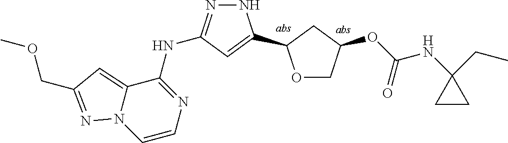 [SMILES: CCC1(NC(=O)O[C@H]2CO[C@@H](c3cc(Nc4nccn5nc(COC)cc45)n[nH]3)C2)CC1] | A | |
| 706 | 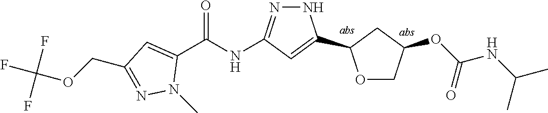 [SMILES: CC(C)NC(=O)O[C@H]1CO[C@@H](c2cc(NC(=O)c3cc(COC(F)(F)F)nn3C)n[nH]2)C1] | A | |
| 707 | 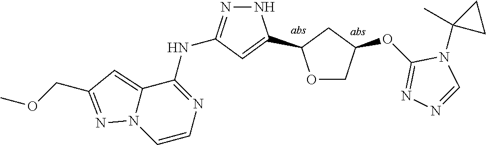 [SMILES: COCc1cc2c(Nc3cc([C@H]4C[C@@H](Oc5nncn5C5(C)CC5)CO4)[nH]n3)nccn2n1] | A | |
| 708 | 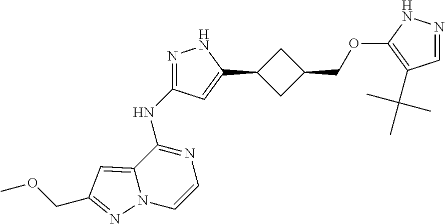 [SMILES: COCc1cc2c(Nc3cc([C@H]4C[C@@H](COc5[nH]ncc5C(C)(C)C)C4)[nH]n3)nccn2n1] | A | |
| 709 | 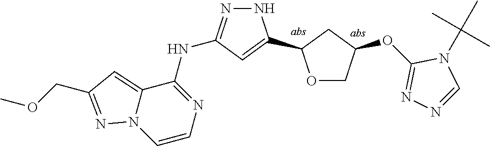 [SMILES: COCc1cc2c(Nc3cc([C@H]4C[C@@H](Oc5nncn5C(C)(C)C)CO4)[nH]n3)nccn2n1] | A | |
| 710 | 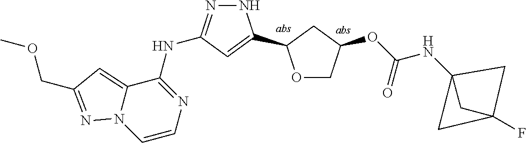 [SMILES: COCc1cc2c(Nc3cc([C@H]4C[C@@H](OC(=O)NC56CC(F)(C5)C6)CO4)[nH]n3)nccn2n1] | A | |
| 711 | 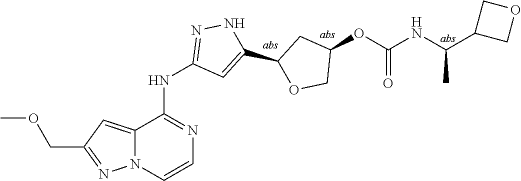 [SMILES: COCc1cc2c(Nc3cc([C@H]4C[C@@H](OC(=O)N[C@H](C)C5COC5)CO4)[nH]n3)nccn2n1] | C | |
| 712 | 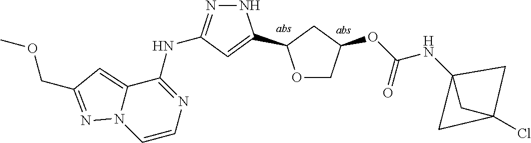 [SMILES: COCc1cc2c(Nc3cc([C@H]4C[C@@H](OC(=O)NC56CC(Cl)(C5)C6)CO4)[nH]n3)nccn2n1] | A | |
| 713 | 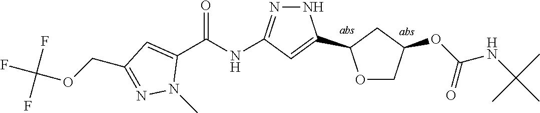 [SMILES: Cn1nc(COC(F)(F)F)cc1C(=O)Nc1cc([C@H]2C[C@@H](OC(=O)NC(C)(C)C)CO2)[nH]n1] | A | |
| 714 | 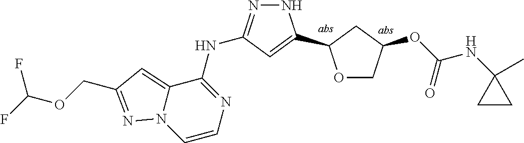 [SMILES: CC1(NC(=O)O[C@H]2CO[C@@H](c3cc(Nc4nccn5nc(COC(F)F)cc45)n[nH]3)C2)CC1] | A | |
| 715 | 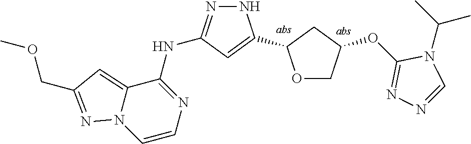 [SMILES: COCc1cc2c(Nc3cc([C@@H]4C[C@H](Oc5nncn5C(C)C)CO4)[nH]n3)nccn2n1] | A | |
| 716 | 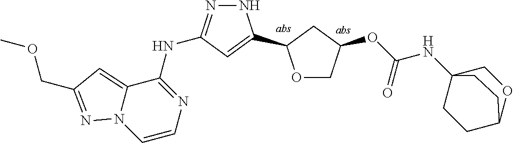 [SMILES: COCc1cc2c(Nc3cc([C@H]4C[C@@H](OC(=O)NC56CCC(CC5)OC6)CO4)[nH]n3)nccn2n1] | A | |
| 717 | 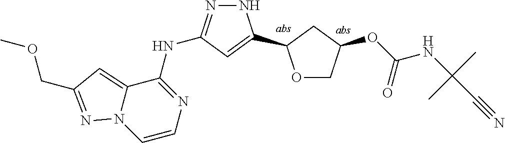 [SMILES: COCc1cc2c(Nc3cc([C@H]4C[C@@H](OC(=O)NC(C)(C)C#N)CO4)[nH]n3)nccn2n1] | A | |
| 718 | 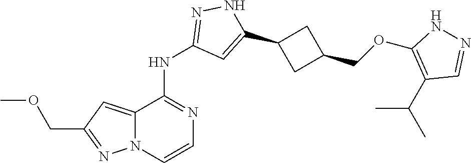 [SMILES: COCc1cc2c(Nc3cc([C@H]4C[C@@H](COc5[nH]ncc5C(C)C)C4)[nH]n3)nccn2n1] | A | |
| 719 | 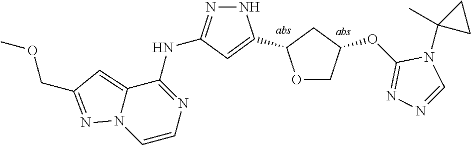 [SMILES: COCc1cc2c(Nc3cc([C@@H]4C[C@H](Oc5nncn5C5(C)CC5)CO4)[nH]n3)nccn2n1] | A | |
| 720 | 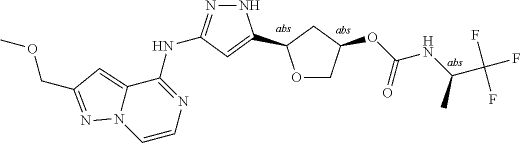 [SMILES: COCc1cc2c(Nc3cc([C@H]4C[C@@H](OC(=O)N[C@H](C)C(F)(F)F)CO4)[nH]n3)nccn2n1] | A | |
| 721 | 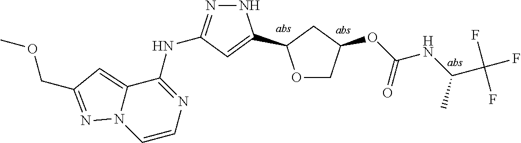 [SMILES: COCc1cc2c(Nc3cc([C@H]4C[C@@H](OC(=O)N[C@@H](C)C(F)(F)F)CO4)[nH]n3)nccn2n1] | A | |
| 722 | 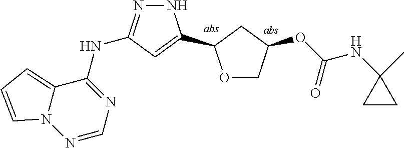 [SMILES: CC1(NC(=O)O[C@H]2CO[C@@H](c3cc(Nc4ncnn5cccc45)n[nH]3)C2)CC1] | A | |
| 723 | 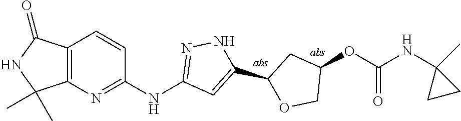 [SMILES: CC1(NC(=O)O[C@H]2CO[C@@H](c3cc(Nc4ccc5c(n4)C(C)(C)NC5=O)n[nH]3)C2)CC1] | A | |
| 724 | 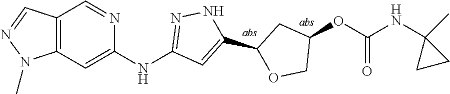 [SMILES: Cn1ncc2cnc(Nc3cc([C@H]4C[C@@H](OC(=O)NC5(C)CC5)CO4)[nH]n3)cc21] | A | |
| 725 | 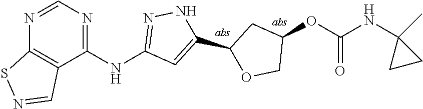 [SMILES: CC1(NC(=O)O[C@H]2CO[C@@H](c3cc(Nc4ncnc5sncc45)n[nH]3)C2)CC1] | A | |
| 726 | 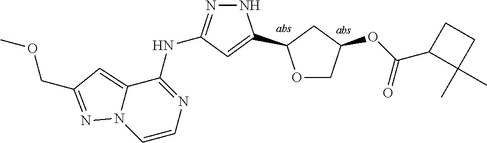 | A | |
| 727 | 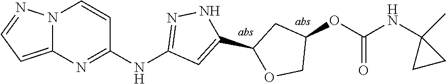 [SMILES: CC1(NC(=O)O[C@H]2CO[C@@H](c3cc(Nc4ccn5nccc5n4)n[nH]3)C2)CC1] | A | |
| 728 | 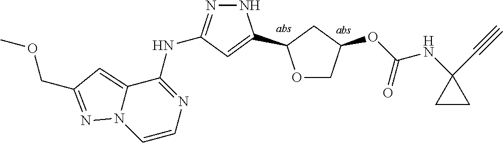 [SMILES: C#CC1(NC(=O)O[C@H]2CO[C@@H](c3cc(Nc4nccn5nc(COC)cc45)n[nH]3)C2)CC1] | A | |
| 729 | 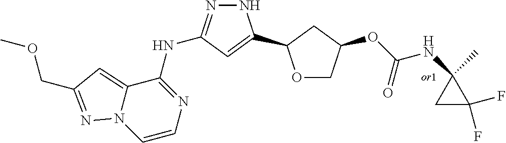 [SMILES: COCc1cc2c(Nc3cc([C@H]4C[C@@H](OC(=O)N[C@]5(C)CC5(F)F)CO4)[nH]n3)nccn2n1] | A | |
| 730 | 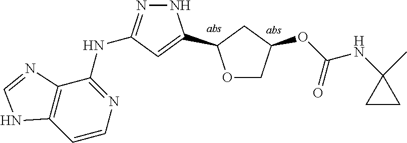 [SMILES: CC1(NC(=O)O[C@H]2CO[C@@H](c3cc(Nc4nccc5[nH]cnc45)n[nH]3)C2)CC1] | C | |
| 731 | 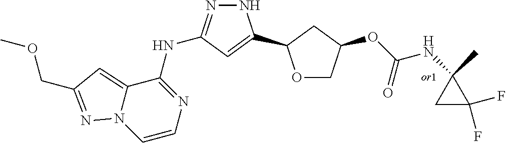 [SMILES: COCc1cc2c(Nc3cc([C@H]4C[C@@H](OC(=O)N[C@@]5(C)CC5(F)F)CO4)[nH]n3)nccn2n1] | A | |
| 732 | 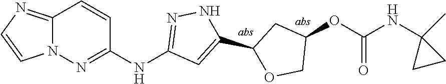 [SMILES: CC1(NC(=O)O[C@H]2CO[C@@H](c3cc(Nc4ccc5nccn5n4)n[nH]3)C2)CC1] | A | |
| 733 | 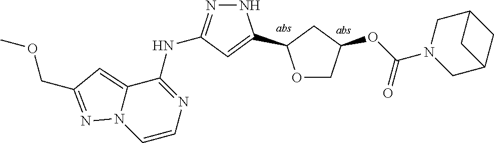 [SMILES: COCc1cc2c(Nc3cc([C@H]4C[C@@H](OC(=O)N5CC6CC(C6)C5)CO4)[nH]n3)nccn2n1] | B | |
| 734 | 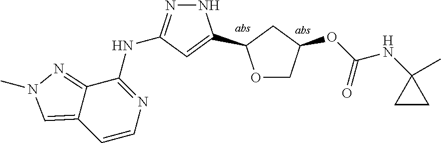 | D | |
| 735 | 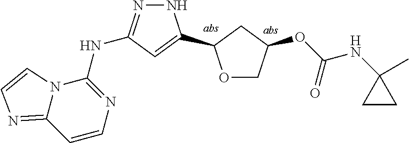 [SMILES: CC1(NC(=O)O[C@H]2CO[C@@H](c3cc(Nc4nccc5nccn45)n[nH]3)C2)CC1] | A | |
| 736 | 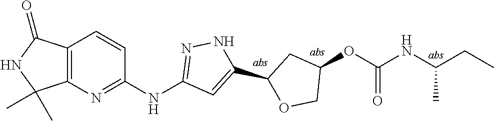 [SMILES: CC[C@H](C)NC(=O)O[C@H]1CO[C@@H](c2cc(Nc3ccc4c(n3)C(C)(C)NC4=O)n[nH]2)C1] | A | |
| 737 | 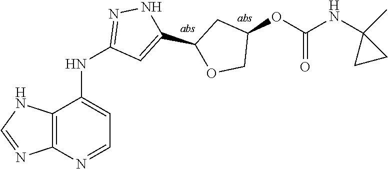 [SMILES: CC1(NC(=O)O[C@H]2CO[C@@H](c3cc(Nc4ccnc5nc[nH]c45)n[nH]3)C2)CC1] | B | |
| 738 | 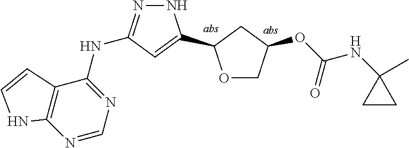 [SMILES: CC1(NC(=O)O[C@H]2CO[C@@H](c3cc(Nc4ncnc5[nH]ccc45)n[nH]3)C2)CC1] | C | |
| 739 | 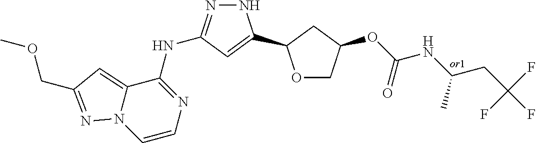 [SMILES: COCc1cc2c(Nc3cc([C@H]4C[C@@H](OC(=O)N[C@@H](C)CC(F)(F)F)CO4)[nH]n3)nccn2n1] | A | |
| 740 | 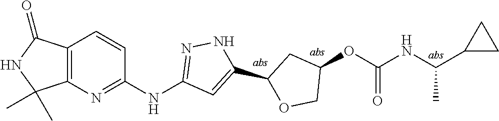 [SMILES: C[C@H](NC(=O)O[C@H]1CO[C@@H](c2cc(Nc3ccc4c(n3)C(C)(C)NC4=O)n[nH]2)C1)C1CC1] | A | |
| 741 | 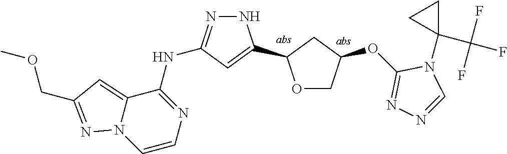 [SMILES: COCc1cc2c(Nc3cc([C@H]4C[C@@H](Oc5nncn5C5(C(F)(F)F)CC5)CO4)[nH]n3)nccn2n1] | B | |
| 742 | 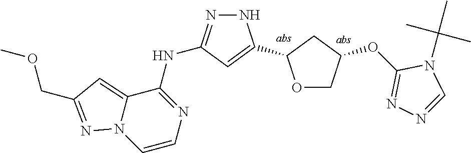 [SMILES: COCc1cc2c(Nc3cc([C@@H]4C[C@H](Oc5nncn5C(C)(C)C)CO4)[nH]n3)nccn2n1] | A | |
| 743 | 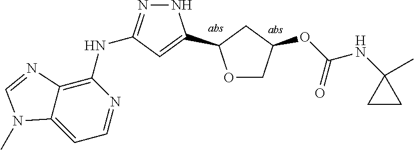 | D | |
| 744 | 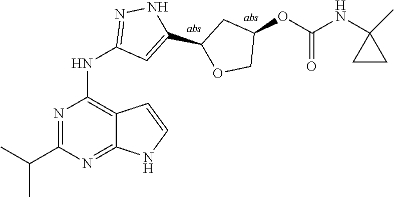 [SMILES: CC(C)c1nc(Nc2cc([C@H]3C[C@@H](OC(=O)NC4(C)CC4)CO3)[nH]n2)c2cc[nH]c2n1] | B | |
| 745 | 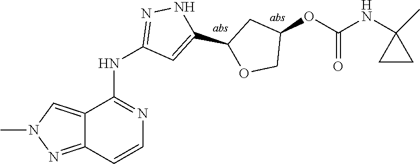 | B | |
| 746 | 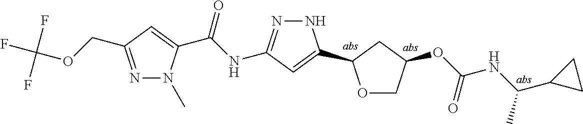 [SMILES: C[C@H](NC(=O)O[C@H]1CO[C@@H](c2cc(NC(=O)c3cc(COC(F)(F)F)nn3C)n[nH]2)C1)C1CC1] | A | |
| 747 | 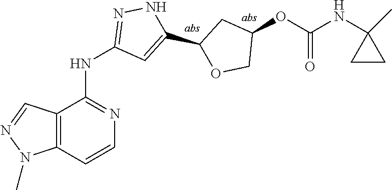 [SMILES: Cn1ncc2c(Nc3cc([C@H]4C[C@@H](OC(=O)NC5(C)CC5)CO4)[nH]n3)nccc21] | C | |
| 748 | 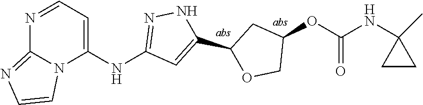 [SMILES: CC1(NC(=O)O[C@H]2CO[C@@H](c3cc(Nc4ccnc5nccn45)n[nH]3)C2)CC1] | A | |
| 749 | 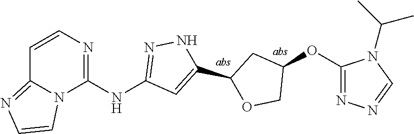 [SMILES: CC(C)n1cnnc1O[C@H]1CO[C@@H](c2cc(Nc3nccc4nccn34)n[nH]2)C1] | B | |
| 750 | 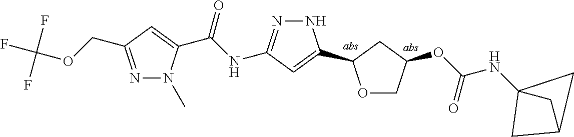 [SMILES: Cn1nc(COC(F)(F)F)cc1C(=O)Nc1cc([C@H]2C[C@@H](OC(=O)NC34CC(C3)C4)CO2)[nH]n1] | A | |
| 751 | 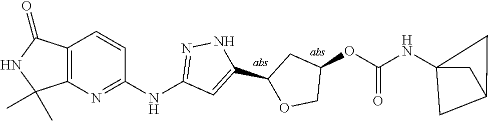 [SMILES: CC1(C)NC(=O)c2ccc(Nc3cc([C@H]4C[C@@H](OC(=O)NC56CC(C5)C6)CO4)[nH]n3)nc21] | A | |
| 752 | 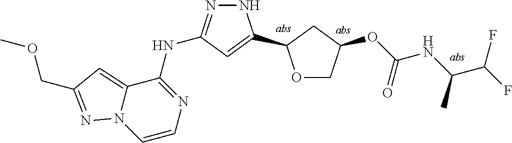 [SMILES: COCc1cc2c(Nc3cc([C@H]4C[C@@H](OC(=O)N[C@H](C)C(F)F)CO4)[nH]n3)nccn2n1] | A | |
| 753 | 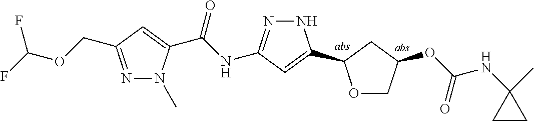 [SMILES: Cn1nc(COC(F)F)cc1C(=O)Nc1cc([C@H]2C[C@@H](OC(=O)NC3(C)CC3)CO2)[nH]n1] | A | |
| 754 | 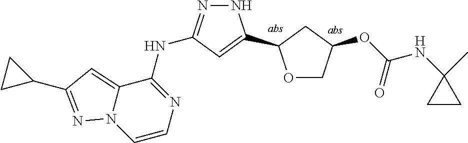 [SMILES: CC1(NC(=O)O[C@H]2CO[C@@H](c3cc(Nc4nccn5nc(C6CC6)cc45)n[nH]3)C2)CC1] | A | |
| 755 | 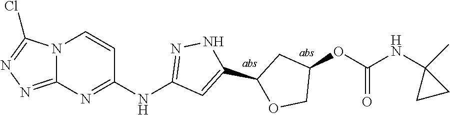 [SMILES: CC1(NC(=O)O[C@H]2CO[C@@H](c3cc(Nc4ccn5c(Cl)nnc5n4)n[nH]3)C2)CC1] | C | |
| 756 | 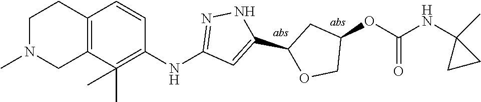 | A | |
| 757 | 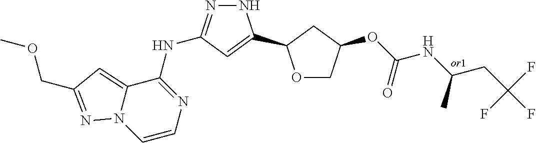 [SMILES: COCc1cc2c(Nc3cc([C@H]4C[C@@H](OC(=O)N[C@H](C)CC(F)(F)F)CO4)[nH]n3)nccn2n1] | A | |
| 758 | 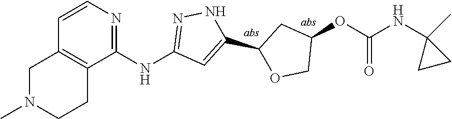 [SMILES: CN1CCc2c(ccnc2Nc2cc([C@H]3C[C@@H](OC(=O)NC4(C)CC4)CO3)[nH]n2)C1] | D | |
| 759 | 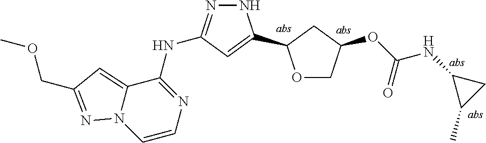 [SMILES: COCc1cc2c(Nc3cc([C@H]4C[C@@H](OC(=O)N[C@@H]5C[C@@H]5C)CO4)[nH]n3)nccn2n1] | A | |
| 760 | 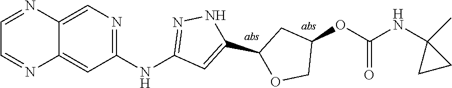 [SMILES: CC1(NC(=O)O[C@H]2CO[C@@H](c3cc(Nc4cc5nccnc5cn4)n[nH]3)C2)CC1] | C | |
| 761 | 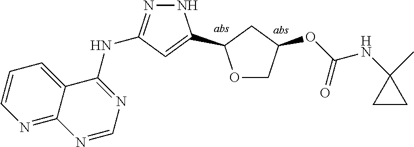 [SMILES: CC1(NC(=O)O[C@H]2CO[C@@H](c3cc(Nc4ncnc5ncccc45)n[nH]3)C2)CC1] | A | |
| 762 | 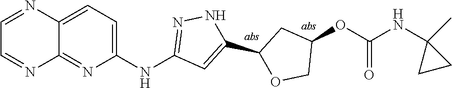 [SMILES: CC1(NC(=O)O[C@H]2CO[C@@H](c3cc(Nc4ccc5nccnc5n4)n[nH]3)C2)CC1] | B | |
| 763 | 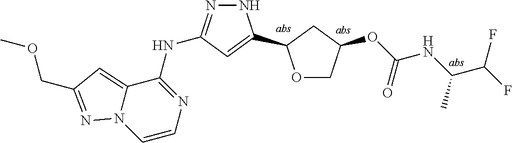 [SMILES: COCc1cc2c(Nc3cc([C@H]4C[C@@H](OC(=O)N[C@@H](C)C(F)F)CO4)[nH]n3)nccn2n1] | A | |
| 764 | 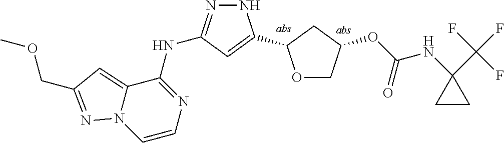 [SMILES: COCc1cc2c(Nc3cc([C@@H]4C[C@H](OC(=O)NC5(C(F)(F)F)CC5)CO4)[nH]n3)nccn2n1] | A | |
| 765 | 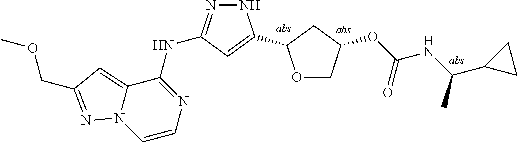 [SMILES: COCc1cc2c(Nc3cc([C@@H]4C[C@H](OC(=O)N[C@H](C)C5CC5)CO4)[nH]n3)nccn2n1] | A | |
| 766 | 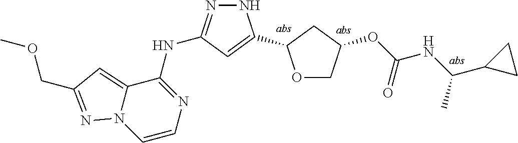 [SMILES: COCc1cc2c(Nc3cc([C@@H]4C[C@H](OC(=O)N[C@@H](C)C5CC5)CO4)[nH]n3)nccn2n1] | A | |
| 767 | 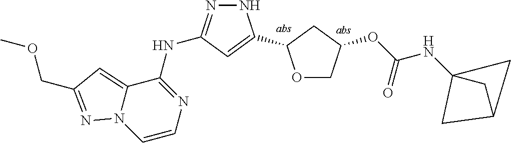 [SMILES: COCc1cc2c(Nc3cc([C@@H]4C[C@H](OC(=O)NC56CC(C5)C6)CO4)[nH]n3)nccn2n1] | A | |
| 768 | 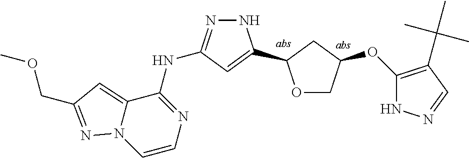 [SMILES: COCc1cc2c(Nc3cc([C@H]4C[C@@H](Oc5[nH]ncc5C(C)(C)C)CO4)[nH]n3)nccn2n1] | A | |
| 769 | 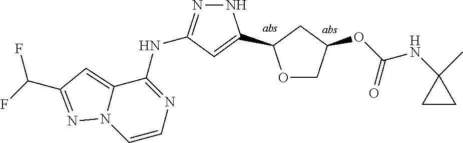 [SMILES: CC1(NC(=O)O[C@H]2CO[C@@H](c3cc(Nc4nccn5nc(C(F)F)cc45)n[nH]3)C2)CC1] | A | |
| 770 | 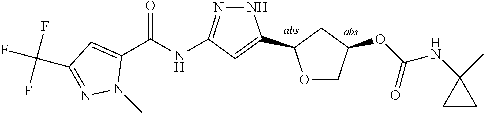 [SMILES: Cn1nc(C(F)(F)F)cc1C(=O)Nc1cc([C@H]2C[C@@H](OC(=O)NC3(C)CC3)CO2)[nH]n1] | A | |
| 771 | 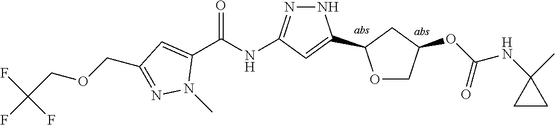 [SMILES: Cn1nc(COCC(F)(F)F)cc1C(=O)Nc1cc([C@H]2C[C@@H](OC(=O)NC3(C)CC3)CO2)[nH]n1] | A | |
| 772 | 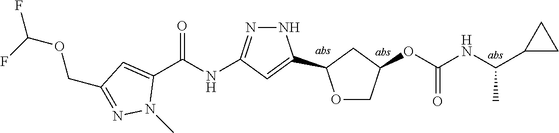 [SMILES: C[C@H](NC(=O)O[C@H]1CO[C@@H](c2cc(NC(=O)c3cc(COC(F)F)nn3C)n[nH]2)C1)C1CC1] | A | |
| 773 | 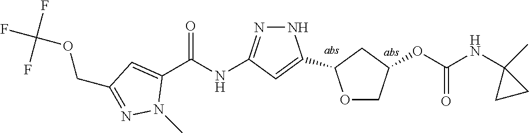 [SMILES: Cn1nc(COC(F)(F)F)cc1C(=O)Nc1cc([C@@H]2C[C@H](OC(=O)NC3(C)CC3)CO2)[nH]n1] | A | |
| 774 | 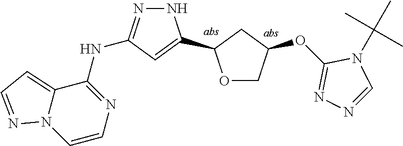 [SMILES: CC(C)(C)n1cnnc1O[C@H]1CO[C@@H](c2cc(Nc3nccn4nccc34)n[nH]2)C1] | A | |
| 775 | 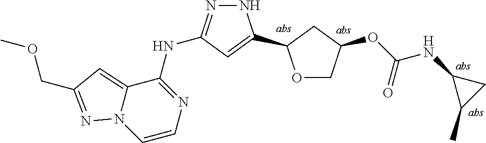 [SMILES: COCc1cc2c(Nc3cc([C@H]4C[C@@H](OC(=O)N[C@H]5C[C@H]5C)CO4)[nH]n3)nccn2n1] | A | |
| 776 | 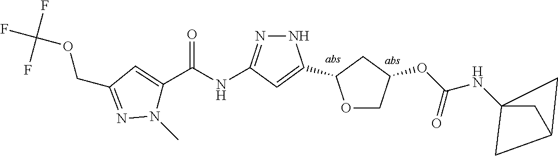 [SMILES: Cn1nc(COC(F)(F)F)cc1C(=O)Nc1cc([C@@H]2C[C@H](OC(=O)NC34CC(C3)C4)CO2)[nH]n1] | A | |
| 778 | 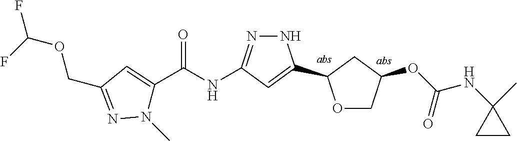 [SMILES: Cn1nc(COC(F)F)cc1C(=O)Nc1cc([C@H]2C[C@@H](OC(=O)NC3(C)CC3)CO2)[nH]n1] | A | |
| 779 | 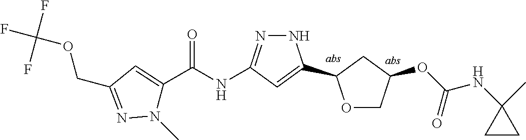 [SMILES: Cn1nc(COC(F)(F)F)cc1C(=O)Nc1cc([C@H]2C[C@@H](OC(=O)NC3(C)CC3)CO2)[nH]n1] | A | |
| 780 | 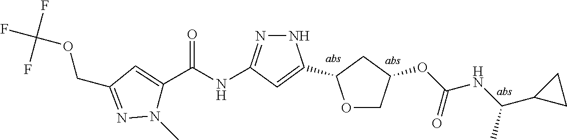 [SMILES: C[C@H](NC(=O)O[C@@H]1CO[C@H](c2cc(NC(=O)c3cc(COC(F)(F)F)nn3C)n[nH]2)C1)C1CC1] | A | |
| 781 | 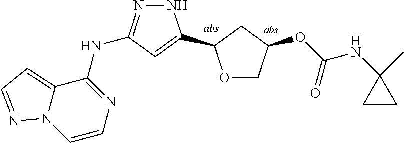 [SMILES: CC1(NC(=O)O[C@H]2CO[C@@H](c3cc(Nc4nccn5nccc45)n[nH]3)C2)CC1] | A | |
| 782 | 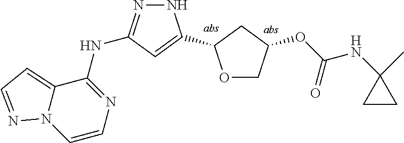 [SMILES: CC1(NC(=O)O[C@@H]2CO[C@H](c3cc(Nc4nccn5nccc45)n[nH]3)C2)CC1] | A | |
| 783 | 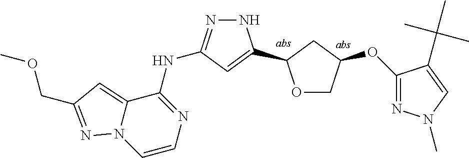 [SMILES: COCc1cc2c(Nc3cc([C@H]4C[C@@H](Oc5nn(C)cc5C(C)(C)C)CO4)[nH]n3)nccn2n1] | D | |
| 784 | 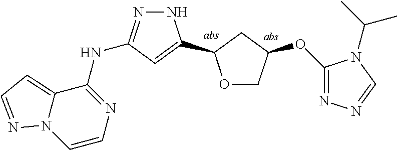 [SMILES: CC(C)n1cnnc1O[C@H]1CO[C@@H](c2cc(Nc3nccn4nccc34)n[nH]2)C1] | A | |
| 785 | 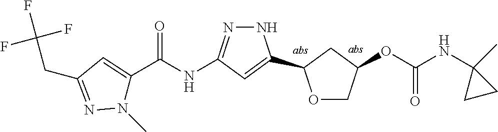 [SMILES: Cn1nc(CC(F)(F)F)cc1C(=O)Nc1cc([C@H]2C[C@@H](OC(=O)NC3(C)CC3)CO2)[nH]n1] | A | |
| 786 | 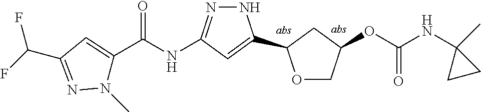 [SMILES: Cn1nc(C(F)F)cc1C(=O)Nc1cc([C@H]2C[C@@H](OC(=O)NC3(C)CC3)CO2)[nH]n1] | A | |
| 787 | 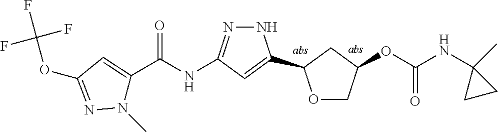 [SMILES: Cn1nc(OC(F)(F)F)cc1C(=O)Nc1cc([C@H]2C[C@@H](OC(=O)NC3(C)CC3)CO2)[nH]n1] | A | |
| 788 | 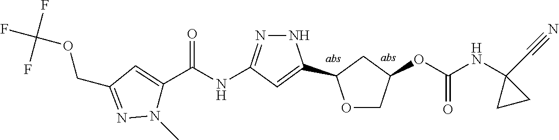 [SMILES: Cn1nc(COC(F)(F)F)cc1C(=O)Nc1cc([C@H]2C[C@@H](OC(=O)NC3(C#N)CC3)CO2)[nH]n1] | A | |
| 789 | 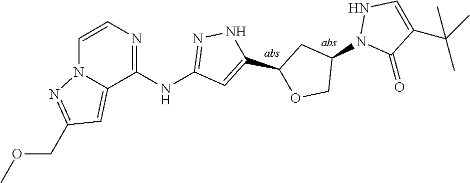 [SMILES: COCc1cc2c(Nc3cc([C@H]4C[C@@H](n5[nH]cc(C(C)(C)C)c5=O)CO4)[nH]n3)nccn2n1] | | |
| 790 | 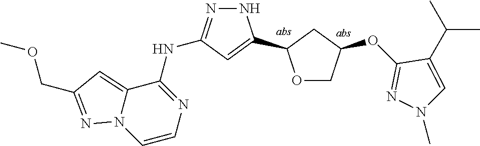 [SMILES: COCc1cc2c(Nc3cc([C@H]4C[C@@H](Oc5nn(C)cc5C(C)C)CO4)[nH]n3)nccn2n1] | D | |
| 791 | 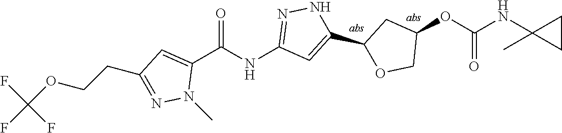 [SMILES: Cn1nc(CCOC(F)(F)F)cc1C(=O)Nc1cc([C@H]2C[C@@H](OC(=O)NC3(C)CC3)CO2)[nH]n1] | A | |
| 792 | 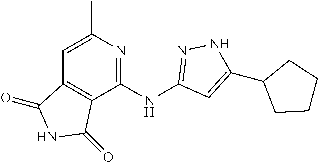 [SMILES: Cc1cc2c(c(Nc3cc(C4CCCC4)[nH]n3)n1)C(=O)NC2=O] | D | |
| 793 | 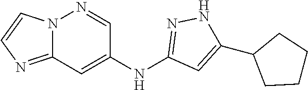 [SMILES: c1cn2ncc(Nc3cc(C4CCCC4)[nH]n3)cc2n1] | D | |
| 794 | 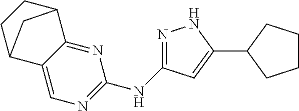 [SMILES: c1c(Nc2ncc3c(n2)C2CCC3C2)n[nH]c1C1CCCC1] | D | |
| 795 | 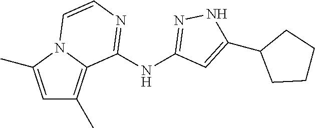 [SMILES: Cc1cc(C)n2ccnc(Nc3cc(C4CCCC4)[nH]n3)c12] | D | |
| 796 | 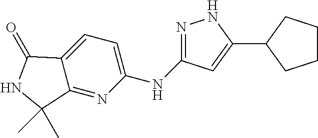 [SMILES: CC1(C)NC(=O)c2ccc(Nc3cc(C4CCCC4)[nH]n3)nc21] | A | |
| 797 | 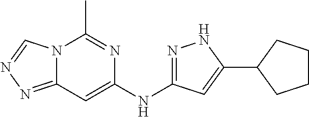 [SMILES: Cc1nc(Nc2cc(C3CCCC3)[nH]n2)cc2nncn12] | D | |
| 798 | 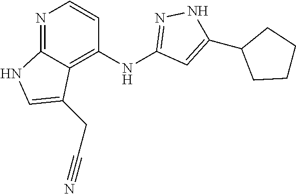 [SMILES: N#CCc1c[nH]c2nccc(Nc3cc(C4CCCC4)[nH]n3)c12] | D | |
| 799 | 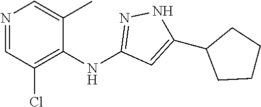 [SMILES: Cc1cncc(Cl)c1Nc1cc(C2CCCC2)[nH]n1] | D | |
| 800 | 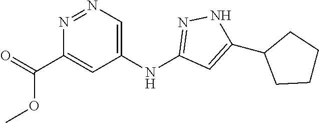 [SMILES: COC(=O)c1cc(Nc2cc(C3CCCC3)[nH]n2)cnn1] | D | |
| 801 | 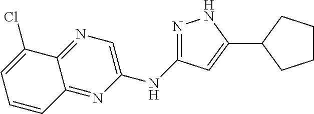 [SMILES: Clc1cccc2nc(Nc3cc(C4CCCC4)[nH]n3)cnc12] | D | |
| 802 | 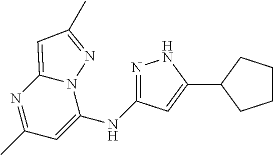 [SMILES: Cc1cc(Nc2cc(C3CCCC3)[nH]n2)n2nc(C)cc2n1] | D | |
| 803 | 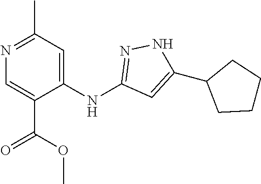 [SMILES: COC(=O)c1cnc(C)cc1Nc1cc(C2CCCC2)[nH]n1] | D | |
| 804 | 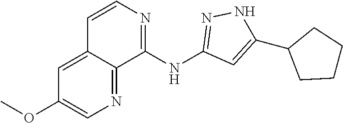 [SMILES: COc1cnc2c(Nc3cc(C4CCCC4)[nH]n3)nccc2c1] | D | |
| 805 | 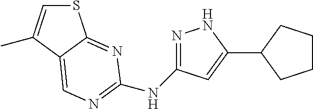 [SMILES: Cc1csc2nc(Nc3cc(C4CCCC4)[nH]n3)ncc12] | D | |
| 806 | 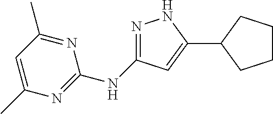 [SMILES: Cc1cc(C)nc(Nc2cc(C3CCCC3)[nH]n2)n1] | D | |
| 807 | 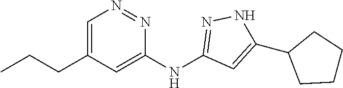 [SMILES: CCCc1cnnc(Nc2cc(C3CCCC3)[nH]n2)c1] | D | |
| 808 | 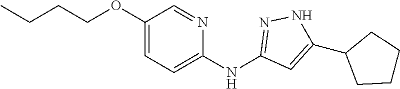 [SMILES: CCCCOc1ccc(Nc2cc(C3CCCC3)[nH]n2)nc1] | D | |
| 809 | 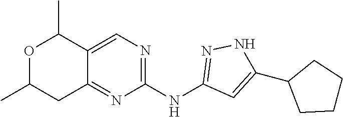 [SMILES: CC1Cc2nc(Nc3cc(C4CCCC4)[nH]n3)ncc2C(C)O1] | D | |
| 810 | 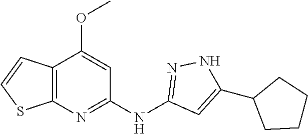 [SMILES: COc1cc(Nc2cc(C3CCCC3)[nH]n2)nc2sccc12] | D | |
| 811 | 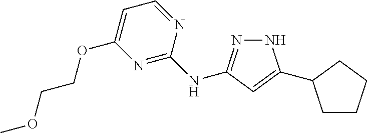 [SMILES: COCCOc1ccnc(Nc2cc(C3CCCC3)[nH]n2)n1] | D | |
| 812 | 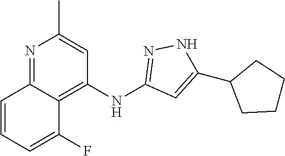 [SMILES: Cc1cc(Nc2cc(C3CCCC3)[nH]n2)c2c(F)cccc2n1] | D | |
| 813 | 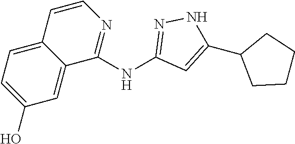 [SMILES: Oc1ccc2ccnc(Nc3cc(C4CCCC4)[nH]n3)c2c1] | D | |
| 814 | 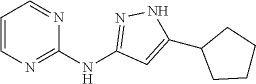 [SMILES: c1cnc(Nc2cc(C3CCCC3)[nH]n2)nc1] | | |
| 815 | 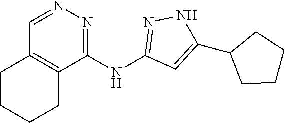 [SMILES: c1c(Nc2nncc3c2CCCC3)n[nH]c1C1CCCC1] | | |
| 816 | 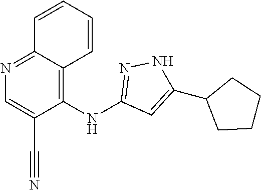 [SMILES: N#Cc1cnc2ccccc2c1Nc1cc(C2CCCC2)[nH]n1] | | |
| 817 | 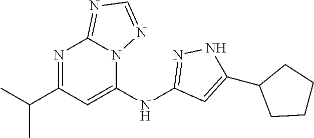 [SMILES: CC(C)c1cc(Nc2cc(C3CCCC3)[nH]n2)n2ncnc2n1] | D | |
| 818 | 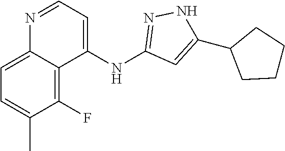 [SMILES: Cc1ccc2nccc(Nc3cc(C4CCCC4)[nH]n3)c2c1F] | | |
| 819 | 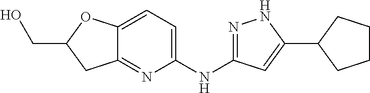 [SMILES: OCC1Cc2nc(Nc3cc(C4CCCC4)[nH]n3)ccc2O1] | | |
| 820 | 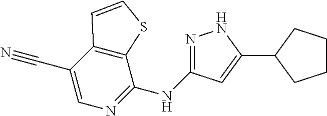 [SMILES: N#Cc1cnc(Nc2cc(C3CCCC3)[nH]n2)c2sccc12] | | |
| 821 | 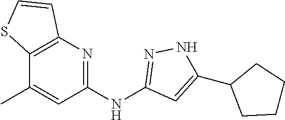 [SMILES: Cc1cc(Nc2cc(C3CCCC3)[nH]n2)nc2ccsc12] | | |
| 822 | 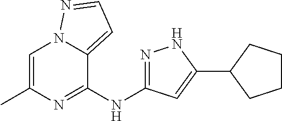 [SMILES: Cc1cn2nccc2c(Nc2cc(C3CCCC3)[nH]n2)n1] | C | |
| 823 | 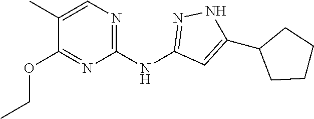 [SMILES: CCOc1nc(Nc2cc(C3CCCC3)[nH]n2)ncc1C] | | |
| 824 | 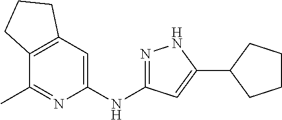 [SMILES: Cc1nc(Nc2cc(C3CCCC3)[nH]n2)cc2c1CCC2] | | |
| 825 | 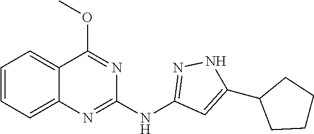 [SMILES: COc1nc(Nc2cc(C3CCCC3)[nH]n2)nc2ccccc12] | | |
| 826 | 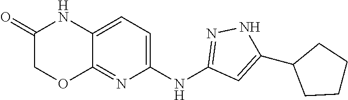 [SMILES: O=C1COc2nc(Nc3cc(C4CCCC4)[nH]n3)ccc2N1] | | |
| 827 | 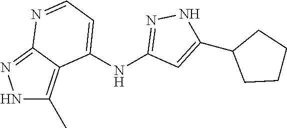 [SMILES: Cc1[nH]nc2nccc(Nc3cc(C4CCCC4)[nH]n3)c12] | D | |
| 828 | 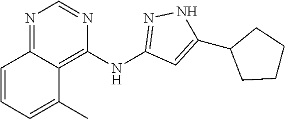 [SMILES: Cc1cccc2ncnc(Nc3cc(C4CCCC4)[nH]n3)c12] | | |
| 829 | 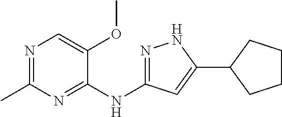 [SMILES: COc1cnc(C)nc1Nc1cc(C2CCCC2)[nH]n1] | | |
| 830 | 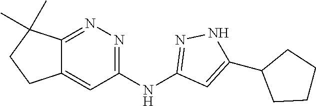 [SMILES: CC1(C)CCc2cc(Nc3cc(C4CCCC4)[nH]n3)nnc21] | | |
| 831 | 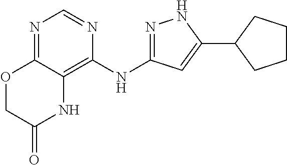 [SMILES: O=C1COc2ncnc(Nc3cc(C4CCCC4)[nH]n3)c2N1] | D | |
| 832 | 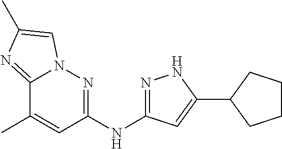 [SMILES: Cc1cn2nc(Nc3cc(C4CCCC4)[nH]n3)cc(C)c2n1] | D | |
| 833 | 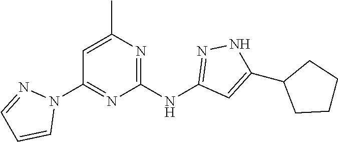 [SMILES: Cc1cc(-n2cccn2)nc(Nc2cc(C3CCCC3)[nH]n2)n1] | | |
| 834 | 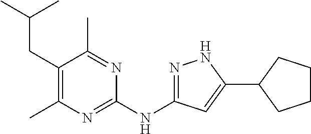 [SMILES: Cc1nc(Nc2cc(C3CCCC3)[nH]n2)nc(C)c1CC(C)C] | | |
| 835 | 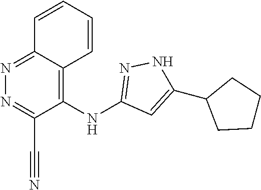 [SMILES: N#Cc1nnc2ccccc2c1Nc1cc(C2CCCC2)[nH]n1] | | |
| 836 | 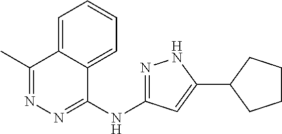 [SMILES: Cc1nnc(Nc2cc(C3CCCC3)[nH]n2)c2ccccc12] | | |
| 837 | 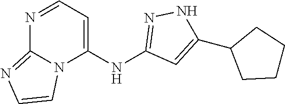 [SMILES: c1cc(Nc2cc(C3CCCC3)[nH]n2)n2ccnc2n1] | D | |
| 838 | 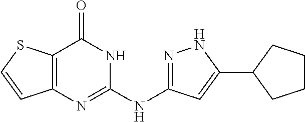 [SMILES: O=c1[nH]c(Nc2cc(C3CCCC3)[nH]n2)nc2ccsc12] | | |
| 839 | 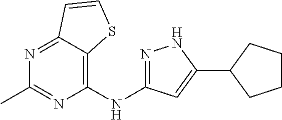 [SMILES: Cc1nc(Nc2cc(C3CCCC3)[nH]n2)c2sccc2n1] | D | |
| 840 | 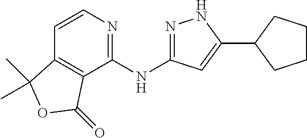 [SMILES: CC1(C)OC(=O)c2c1ccnc2Nc1cc(C2CCCC2)[nH]n1] | | |
| 841 | 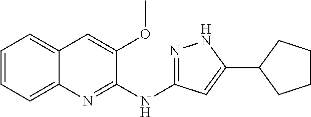 [SMILES: COc1cc2ccccc2nc1Nc1cc(C2CCCC2)[nH]n1] | | |
| 842 | 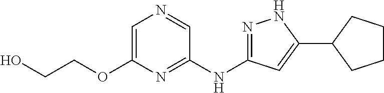 [SMILES: OCCOc1cncc(Nc2cc(C3CCCC3)[nH]n2)n1] | B | |
| 843 | 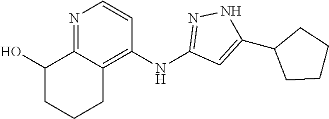 [SMILES: OC1CCCc2c(Nc3cc(C4CCCC4)[nH]n3)ccnc21] | | |
| 844 | 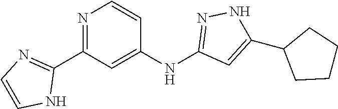 [SMILES: c1cc(Nc2cc(C3CCCC3)[nH]n2)cc(-c2ncc[nH]2)n1] | | |
| 845 | 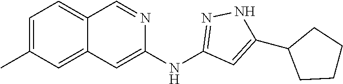 [SMILES: Cc1ccc2cnc(Nc3cc(C4CCCC4)[nH]n3)cc2c1] | | |
| 846 | 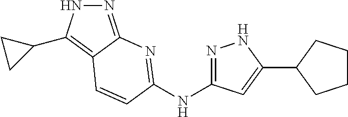 [SMILES: c1cc2c(C3CC3)[nH]nc2nc1Nc1cc(C2CCCC2)[nH]n1] | | |
| 847 | 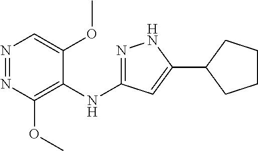 [SMILES: COc1cnnc(OC)c1Nc1cc(C2CCCC2)[nH]n1] | | |
| 848 | 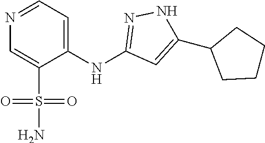 [SMILES: NS(=O)(=O)c1cnccc1Nc1cc(C2CCCC2)[nH]n1] | | |
| 849 | 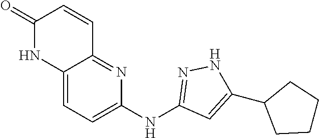 [SMILES: O=c1ccc2nc(Nc3cc(C4CCCC4)[nH]n3)ccc2[nH]1] | D | |
| 850 | 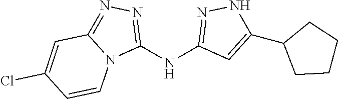 [SMILES: Clc1ccn2c(Nc3cc(C4CCCC4)[nH]n3)nnc2c1] | D | |
| 851 | 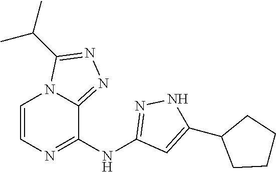 [SMILES: CC(C)c1nnc2c(Nc3cc(C4CCCC4)[nH]n3)nccn12] | | |
| 852 | 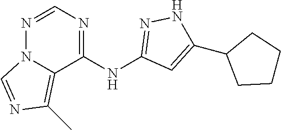 [SMILES: Cc1ncn2ncnc(Nc3cc(C4CCCC4)[nH]n3)c12] | | |
| 853 | 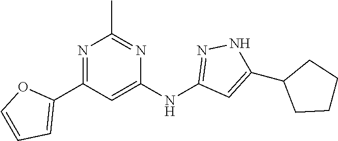 [SMILES: Cc1nc(Nc2cc(C3CCCC3)[nH]n2)cc(-c2ccco2)n1] | D | |
| 854 | 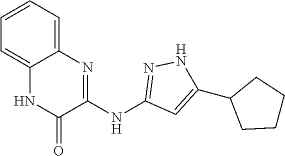 [SMILES: O=c1[nH]c2ccccc2nc1Nc1cc(C2CCCC2)[nH]n1] | | |
| 855 | 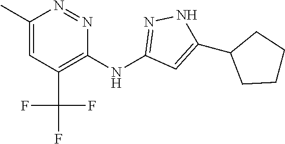 [SMILES: Cc1cc(C(F)(F)F)c(Nc2cc(C3CCCC3)[nH]n2)nn1] | D | |
| 856 | 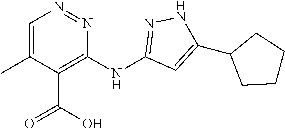 [SMILES: Cc1cnnc(Nc2cc(C3CCCC3)[nH]n2)c1C(=O)O] | D | |
| 857 | 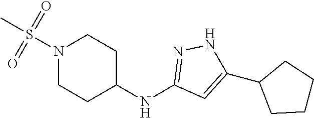 [SMILES: CS(=O)(=O)N1CCC(Nc2cc(C3CCCC3)[nH]n2)CC1] | D | |
| 858 | 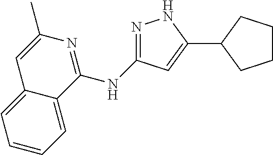 [SMILES: Cc1cc2ccccc2c(Nc2cc(C3CCCC3)[nH]n2)n1] | D | |
| 859 | 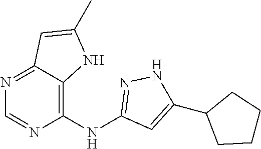 [SMILES: Cc1cc2ncnc(Nc3cc(C4CCCC4)[nH]n3)c2[nH]1] | D | |
| 860 | 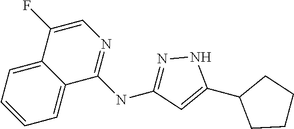 [SMILES: Fc1cnc(Nc2cc(C3CCCC3)[nH]n2)c2ccccc12] | D | |
| 861 | 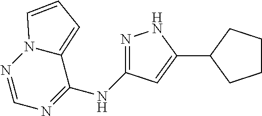 [SMILES: c1cc2c(Nc3cc(C4CCCC4)[nH]n3)ncnn2c1] | D | |
| 862 | 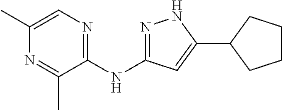 [SMILES: Cc1cnc(Nc2cc(C3CCCC3)[nH]n2)c(C)n1] | D | |
| 863 | 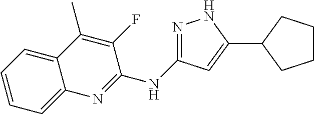 [SMILES: Cc1c(F)c(Nc2cc(C3CCCC3)[nH]n2)nc2ccccc12] | D | |
| 864 | 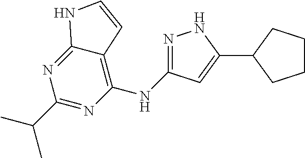 [SMILES: CC(C)c1nc(Nc2cc(C3CCCC3)[nH]n2)c2cc[nH]c2n1] | C | |
| 865 | 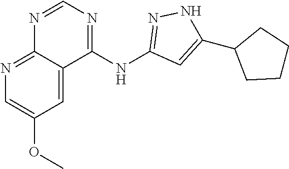 [SMILES: COc1cnc2ncnc(Nc3cc(C4CCCC4)[nH]n3)c2c1] | D | |
| 866 | 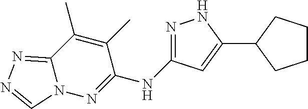 [SMILES: Cc1c(Nc2cc(C3CCCC3)[nH]n2)nn2cnnc2c1C] | D | |
| 867 | 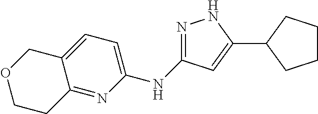 [SMILES: c1cc2c(nc1Nc1cc(C3CCCC3)[nH]n1)CCOC2] | D | |
| 868 | 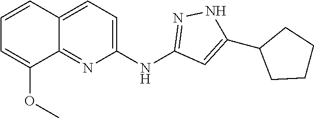 [SMILES: COc1cccc2ccc(Nc3cc(C4CCCC4)[nH]n3)nc12] | D | |
| 869 | 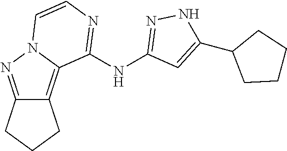 [SMILES: c1cn2nc3c(c2c(Nc2cc(C4CCCC4)[nH]n2)n1)CCC3] | D | |
| 870 | 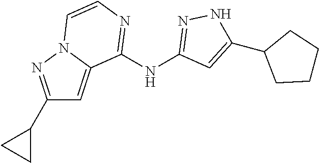 [SMILES: c1cn2nc(C3CC3)cc2c(Nc2cc(C3CCCC3)[nH]n2)n1] | D | |
| 871 | 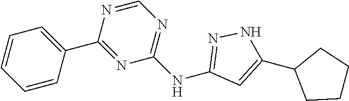 [SMILES: c1ccc(-c2ncnc(Nc3cc(C4CCCC4)[nH]n3)n2)cc1] | D | |
| 872 | 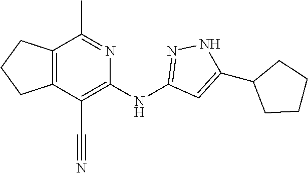 [SMILES: Cc1nc(Nc2cc(C3CCCC3)[nH]n2)c(C#N)c2c1CCC2] | D | |
| 873 | 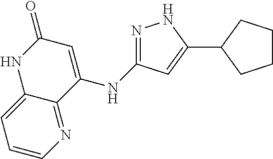 [SMILES: O=c1cc(Nc2cc(C3CCCC3)[nH]n2)c2ncccc2[nH]1] | D | |
| 874 | 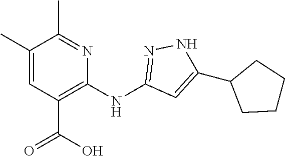 [SMILES: Cc1cc(C(=O)O)c(Nc2cc(C3CCCC3)[nH]n2)nc1C] | D | |
| 875 | 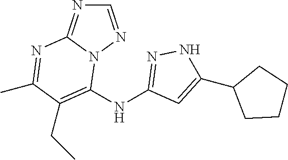 [SMILES: CCc1c(C)nc2ncnn2c1Nc1cc(C2CCCC2)[nH]n1] | D | |
| 876 | 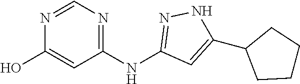 [SMILES: Oc1cc(Nc2cc(C3CCCC3)[nH]n2)ncn1] | D | |
| 877 | 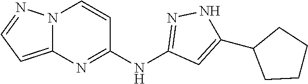 [SMILES: c1cc2nc(Nc3cc(C4CCCC4)[nH]n3)ccn2n1] | C | |
| 878 | 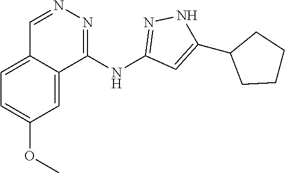 [SMILES: COc1ccc2cnnc(Nc3cc(C4CCCC4)[nH]n3)c2c1] | D | |
| 879 | 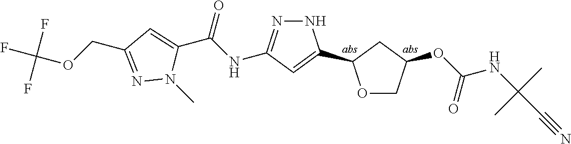 [SMILES: Cn1nc(COC(F)(F)F)cc1C(=O)Nc1cc([C@H]2C[C@@H](OC(=O)NC(C)(C)C#N)CO2)[nH]n1] | A | |
| 880 | 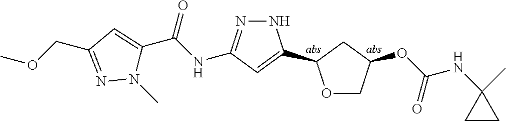 | A | |
| 881 | 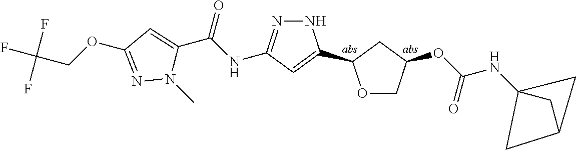 [SMILES: Cn1nc(OCC(F)(F)F)cc1C(=O)Nc1cc([C@H]2C[C@@H](OC(=O)NC34CC(C3)C4)CO2)[nH]n1] | A | |
| 882 | 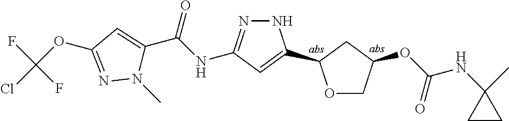 [SMILES: Cn1nc(OC(F)(F)Cl)cc1C(=O)Nc1cc([C@H]2C[C@@H](OC(=O)NC3(C)CC3)CO2)[nH]n1] | A | |
| 883 | 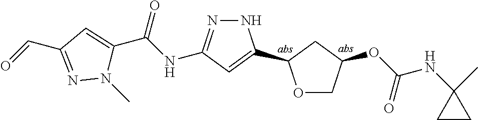 [SMILES: Cn1nc(C=O)cc1C(=O)Nc1cc([C@H]2C[C@@H](OC(=O)NC3(C)CC3)CO2)[nH]n1] | A | |
| 884 | 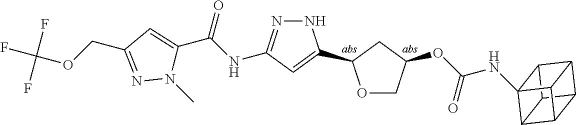 [SMILES: Cn1nc(COC(F)(F)F)cc1C(=O)Nc1cc([C@H]2C[C@@H](OC(=O)NC34C5C6C7C5C3C7C64)CO2)[nH]n1] | A | |
| 885 | 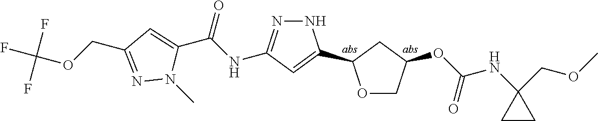 [SMILES: COCC1(NC(=O)O[C@H]2CO[C@@H](c3cc(NC(=O)c4cc(COC(F)(F)F)nn4C)n[nH]3)C2)CC1] | A | |
| 886 | 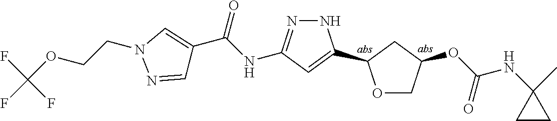 [SMILES: CC1(NC(=O)O[C@H]2CO[C@@H](c3cc(NC(=O)c4cnn(CCOC(F)(F)F)c4)n[nH]3)C2)CC1] | A | |
| 887 | 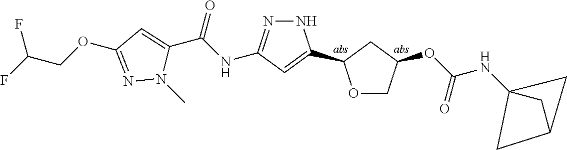 [SMILES: Cn1nc(OCC(F)F)cc1C(=O)Nc1cc([C@H]2C[C@@H](OC(=O)NC34CC(C3)C4)CO2)[nH]n1] | A | |
| 888 | 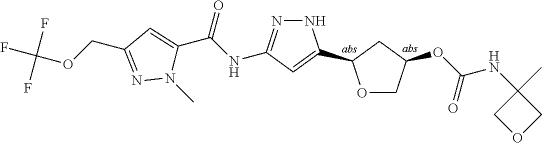 [SMILES: Cn1nc(COC(F)(F)F)cc1C(=O)Nc1cc([C@H]2C[C@@H](OC(=O)NC3(C)COC3)CO2)[nH]n1] | A | |
| 889 | 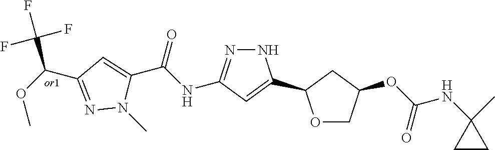 [SMILES: CO[C@H](c1cc(C(=O)Nc2cc([C@H]3C[C@@H](OC(=O)NC4(C)CC4)CO3)[nH]n2)n(C)n1)C(F)(F)F] | B | |
| 890 | 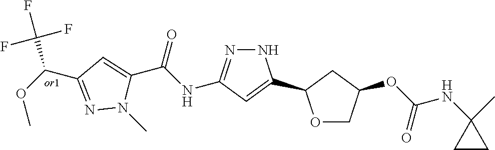 [SMILES: CO[C@@H](c1cc(C(=O)Nc2cc([C@H]3C[C@@H](OC(=O)NC4(C)CC4)CO3)[nH]n2)n(C)n1)C(F)(F)F] | A | |
| 891 | 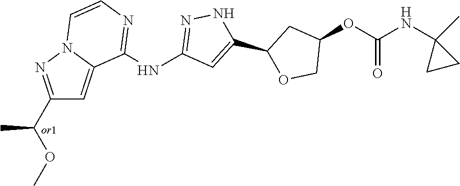 [SMILES: CO[C@@H](C)c1cc2c(Nc3cc([C@H]4C[C@@H](OC(=O)NC5(C)CC5)CO4)[nH]n3)nccn2n1] | A | |
| 892 | 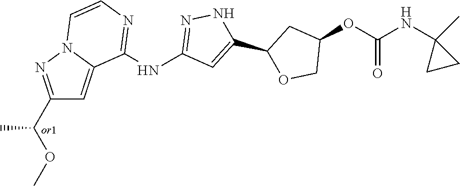 [SMILES: CO[C@H](C)c1cc2c(Nc3cc([C@H]4C[C@@H](OC(=O)NC5(C)CC5)CO4)[nH]n3)nccn2n1] | A | |
| 893 | 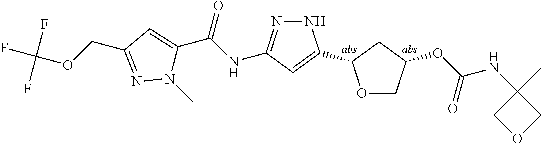 [SMILES: Cn1nc(COC(F)(F)F)cc1C(=O)Nc1cc([C@@H]2C[C@H](OC(=O)NC3(C)COC3)CO2)[nH]n1] | A | |
| 894 | 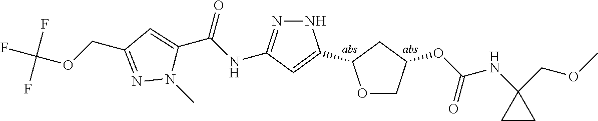 [SMILES: COCC1(NC(=O)O[C@@H]2CO[C@H](c3cc(NC(=O)c4cc(COC(F)(F)F)nn4C)n[nH]3)C2)CC1] | A | |
| 895 | 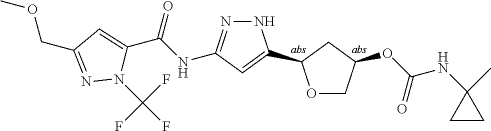 | A | |
| 896 | 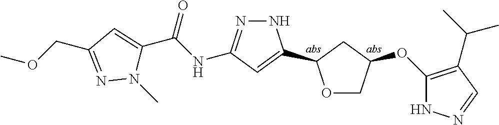 [SMILES: COCc1cc(C(=O)Nc2cc([C@H]3C[C@@H](Oc4[nH]ncc4C(C)C)CO3)[nH]n2)n(C)n1] | A | |
| 897 | 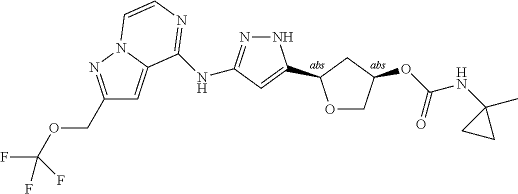 [SMILES: CC1(NC(=O)O[C@H]2CO[C@@H](c3cc(Nc4nccn5nc(COC(F)(F)F)cc45)n[nH]3)C2)CC1] | A | |
| 898 | 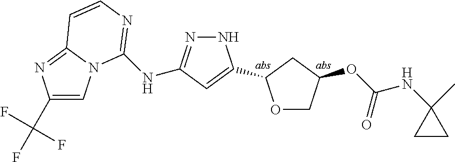 [SMILES: CC1(NC(=O)O[C@H]2CO[C@H](c3cc(Nc4nccc5nc(C(F)(F)F)cn45)n[nH]3)C2)CC1] | C | |
| 899 | 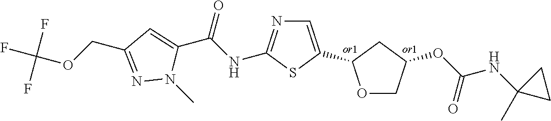 [SMILES: Cn1nc(COC(F)(F)F)cc1C(=O)Nc1ncc([C@@H]2C[C@H](OC(=O)NC3(C)CC3)CO2)s1] | A | |
| 900 | 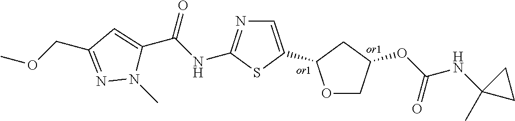 [SMILES: COCc1cc(C(=O)Nc2ncc([C@@H]3C[C@H](OC(=O)NC4(C)CC4)CO3)s2)n(C)n1] | A | |
| 901 | 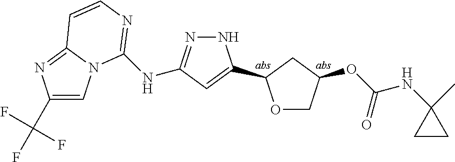 [SMILES: CC1(NC(=O)O[C@H]2CO[C@@H](c3cc(Nc4nccc5nc(C(F)(F)F)cn45)n[nH]3)C2)CC1] | A | |
| 902 | 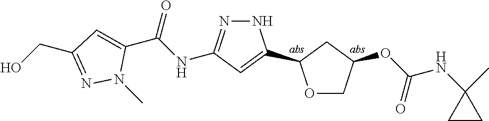 [SMILES: Cn1nc(CO)cc1C(=O)Nc1cc([C@H]2C[C@@H](OC(=O)NC3(C)CC3)CO2)[nH]n1] | C | |
| 903 | 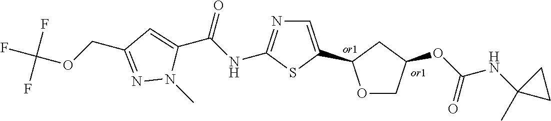 [SMILES: Cn1nc(COC(F)(F)F)cc1C(=O)Nc1ncc([C@H]2C[C@@H](OC(=O)NC3(C)CC3)CO2)s1] | A | |
| 904 | 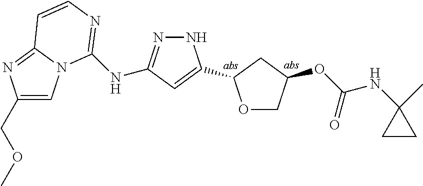 [SMILES: COCc1cn2c(Nc3cc([C@@H]4C[C@@H](OC(=O)NC5(C)CC5)CO4)[nH]n3)nccc2n1] | D | |
| 905 | 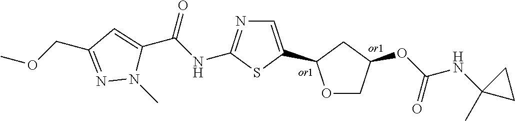 [SMILES: COCc1cc(C(=O)Nc2ncc([C@H]3C[C@@H](OC(=O)NC4(C)CC4)CO3)s2)n(C)n1] | A | |
| 906 | 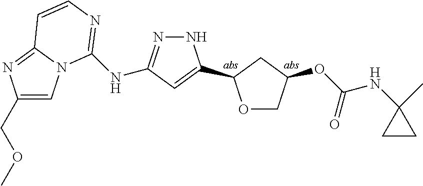 [SMILES: COCc1cn2c(Nc3cc([C@H]4C[C@@H](OC(=O)NC5(C)CC5)CO4)[nH]n3)nccc2n1] | A | |
| 907 | 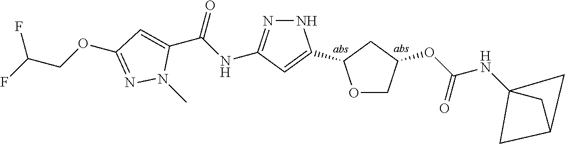 [SMILES: Cn1nc(OCC(F)F)cc1C(=O)Nc1cc([C@@H]2C[C@H](OC(=O)NC34CC(C3)C4)CO2)[nH]n1] | A | |
| 908 | 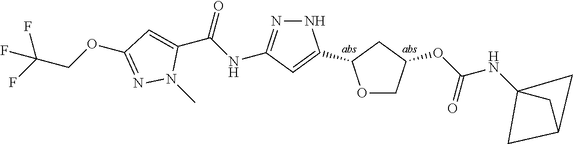 [SMILES: Cn1nc(OCC(F)(F)F)cc1C(=O)Nc1cc([C@@H]2C[C@H](OC(=O)NC34CC(C3)C4)CO2)[nH]n1] | A | |
| 909 | 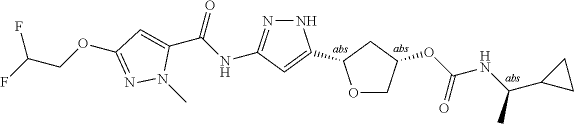 [SMILES: C[C@@H](NC(=O)O[C@@H]1CO[C@H](c2cc(NC(=O)c3cc(OCC(F)F)nn3C)n[nH]2)C1)C1CC1] | A | |
| 910 | 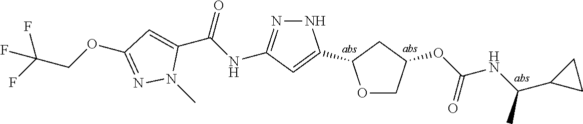 [SMILES: C[C@@H](NC(=O)O[C@@H]1CO[C@H](c2cc(NC(=O)c3cc(OCC(F)(F)F)nn3C)n[nH]2)C1)C1CC1] | A | |
| 911 | 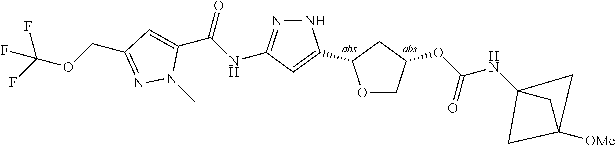 [SMILES: COC12CC(NC(=O)O[C@@H]3CO[C@H](c4cc(NC(=O)c5cc(COC(F)(F)F)nn5C)n[nH]4)C3)(C1)C2] | A | |
| 912 | 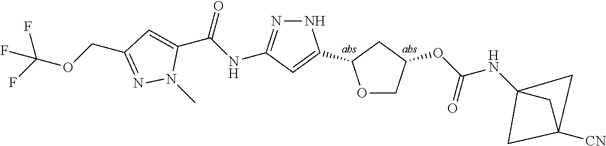 [SMILES: Cn1nc(COC(F)(F)F)cc1C(=O)Nc1cc([C@@H]2C[C@H](OC(=O)NC34CC(C#N)(C3)C4)CO2)[nH]n1] | A | |
| 913 | 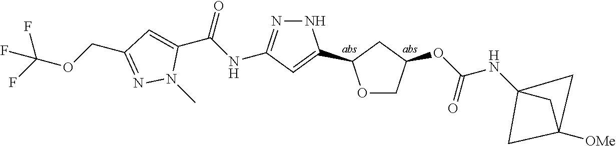 [SMILES: COC12CC(NC(=O)O[C@H]3CO[C@@H](c4cc(NC(=O)c5cc(COC(F)(F)F)nn5C)n[nH]4)C3)(C1)C2] | A | |
| 914 | 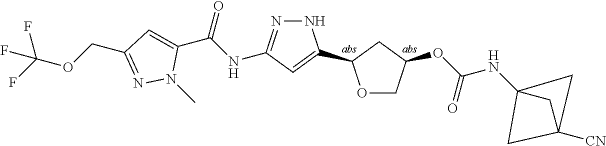 [SMILES: Cn1nc(COC(F)(F)F)cc1C(=O)Nc1cc([C@H]2C[C@@H](OC(=O)NC34CC(C#N)(C3)C4)CO2)[nH]n1] | A | |
| 915 | 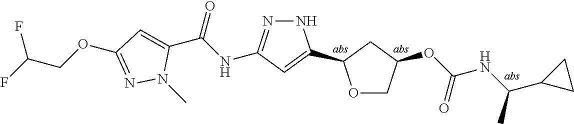 [SMILES: C[C@@H](NC(=O)O[C@H]1CO[C@@H](c2cc(NC(=O)c3cc(OCC(F)F)nn3C)n[nH]2)C1)C1CC1] | A | |
| 916 | 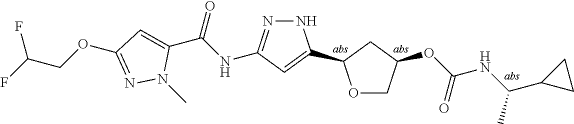 [SMILES: C[C@H](NC(=O)O[C@H]1CO[C@@H](c2cc(NC(=O)c3cc(OCC(F)F)nn3C)n[nH]2)C1)C1CC1] | A | |
| 917 | 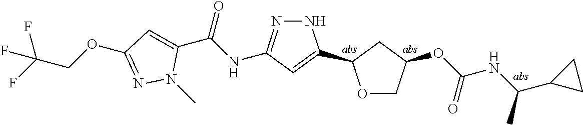 [SMILES: C[C@@H](NC(=O)O[C@H]1CO[C@@H](c2cc(NC(=O)c3cc(OCC(F)(F)F)nn3C)n[nH]2)C1)C1CC1] | A | |
| 918 | 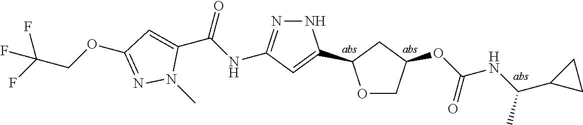 [SMILES: C[C@H](NC(=O)O[C@H]1CO[C@@H](c2cc(NC(=O)c3cc(OCC(F)(F)F)nn3C)n[nH]2)C1)C1CC1] | A | |
| 919 | 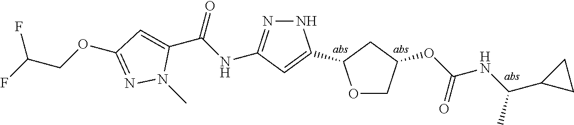 [SMILES: C[C@H](NC(=O)O[C@@H]1CO[C@H](c2cc(NC(=O)c3cc(OCC(F)F)nn3C)n[nH]2)C1)C1CC1] | A | |
| 920 | 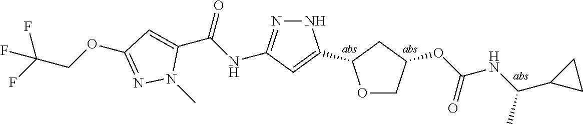 [SMILES: C[C@H](NC(=O)O[C@@H]1CO[C@H](c2cc(NC(=O)c3cc(OCC(F)(F)F)nn3C)n[nH]2)C1)C1CC1] | A | |
| 921 | 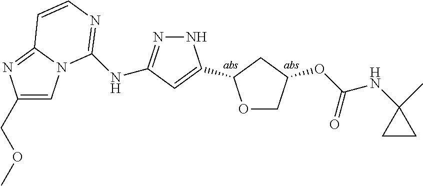 [SMILES: COCc1cn2c(Nc3cc([C@@H]4C[C@H](OC(=O)NC5(C)CC5)CO4)[nH]n3)nccc2n1] | A | |
| 922 | 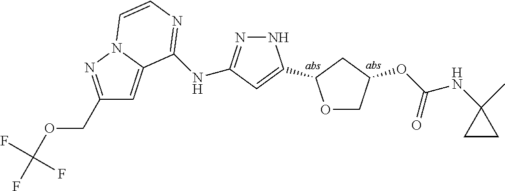 [SMILES: CC1(NC(=O)O[C@@H]2CO[C@H](c3cc(Nc4nccn5nc(COC(F)(F)F)cc45)n[nH]3)C2)CC1] | A | |

In chemical structures in Table 1, above, when a stereocenter is depicted with a dashed or wedged bond and labeled “abs” (or unlabeled), the compound is essentially a single isomer at that stereocenter (rather than an equimolar mixture), and the absolute stereochemistry is as shown in the chemical structure. (See, for example, the structure of Example 5.) When a stereocenter is depicted with a dashed or wedged bond and also labeled “or1” or “or2,” the compound is a single isomer at that stereocenter, but the absolute stereochemistry at that stereocenter has not been determined. (See, for example, the structure of Example 27.) When a stereocenter is depicted with a dashed or wedged bond and also labeled “andl” or “&1”, the compound is a mixture of two isomers at that stereocenter: the structure as drawn, and the isomer in which that stereogenic center has the opposite configuration. (See, for example, the structure of Example 3.)

Certain compounds depicted in Table 1, above, exist in solution at room temperature as non-interconverting atropisomers across a biaryl bond. When one of the atoms of a biaryl bond is labeled as “or1”, this signifies that the compound exists in solution at room temperature as non-interconverting atropisomers, and the compound is essentially a single atropisomer (rather than an equimolar mixture).

In some embodiments, the present disclosure provides a compound in Table 1, above, wherein the compound is denoted as having a Biochemical CDK2 Caliper IC₅₀ of “A”. In some embodiments, the present disclosure provides a compound in Table 1, above, wherein the compound is denoted as having a Biochemical CDK2 Caliper IC₅₀ of “A” or “B”. In some embodiments, the present disclosure provides a compound in Table 1, above, wherein the compound is denoted as having a Biochemical CDK2 Caliper IC₅₀ of “A” or “B” or “C”.

In some embodiments, the present disclosure provides a compound in Table 1, above, wherein the compound is denoted as having a Cell nanoBRET IC₅₀ of “A”. In some embodiments, the present disclosure provides a compound in Table 1, above, wherein the compound is denoted as having a Cell nanoBRET IC₅₀ of “A” or “B”. In some embodiments, the present disclosure provides a compound in Table 1, above, wherein the compound is denoted as having a Cell nanoBRET IC₅₀ of “A” or “B” or “C”.

### 4. General Methods of Providing the Present Compounds

The compounds described herein may be prepared or isolated in general by synthetic and/or semi-synthetic methods known to those skilled in the art for analogous compounds and by methods described in detail in the Examples, herein.

### 5. Uses, Formulation, and Administration

### Pharmaceutically Acceptable Compositions

According to another embodiment, the present disclosure provides a composition comprising a compound described herein, or a pharmaceutically acceptable derivative thereof, and a pharmaceutically acceptable carrier, adjuvant, or vehicle. In some embodiments, the present disclosure provides a pharmaceutical composition comprising a compound described herein, and a pharmaceutically acceptable carrier. The amount of compound in compositions described herein is such that is effective to measurably inhibit a CDK2 protein kinase, or a mutant thereof, in a biological sample or in a patient. In certain embodiments, the amount of compound in compositions described herein is such that it is effective to measurably inhibit a CDK2 protein kinase, or a mutant thereof, in a biological sample or in a patient. In certain embodiments, a composition described herein is formulated for administration to a patient in need of such composition. In some embodiments, a composition described herein is formulated for oral administration to a patient.

The terms “subject” and “patient,” as used herein, means an animal (i.e., a member of the kingdom animal), preferably a mammal, and most preferably a human. In some embodiments, the subject is a human, mouse, rat, cat, monkey, dog, horse, or pig. In some embodiments, the subject is a human. In some embodiments, the subject is a mouse, rat, cat, monkey, dog, horse, or pig.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound described herein that, upon administration to a recipient, is capable of providing, either directly or indirectly, a compound described herein or an inhibitorily active metabolite or residue thereof.

As used herein, the term “inhibitorily active metabolite or residue thereof” means that a metabolite or residue thereof is also an inhibitor of a CDK2 protein kinase, or a mutant thereof.

Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions described herein may be administered in the form of suppositories for rectal or vaginal administration. These can be prepared by mixing the agent with a suitable non-irritating excipient that is solid at room temperature but liquid at rectal or vaginal temperature and therefore will melt in the rectum or vagina to release the drug. Such materials include cocoa butter, beeswax and polyethylene glycols.

Pharmaceutically acceptable compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Preferably, pharmaceutically acceptable compositions described herein are formulated for oral administration. Such formulations may be administered with or without food. In some embodiments, pharmaceutically acceptable compositions described herein are administered without food. In other embodiments, pharmaceutically acceptable compositions described herein are administered with food.

The amount of compounds described herein that may be combined with the carrier materials to produce a composition in a single dosage form will vary depending upon the patient treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

The precise dose to be employed in the compositions will also depend on the route of administration and should be decided according to the judgment of the practitioner and each subject's circumstances. In specific embodiments of the disclosure, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day. It will be recognized that any of the dosages listed herein may constitute an upper or lower dosage range and may be combined with any other dosage to constitute a dosage range comprising an upper and lower limit.

In some embodiments, pharmaceutically acceptable compositions contain a provided compound and/or a pharmaceutically acceptable salt thereof at a concentration ranging from about 0.01 to about 90 wt %, about 0.01 to about 80 wt %, about 0.01 to about 70 wt %, about 0.01 to about 60 wt %, about 0.01 to about 50 wt %, about 0.01 to about 40 wt %, about 0.01 to about 30 wt %, about 0.01 to about 20 wt %, about 0.01 to about 2.0 wt %, about 0.01 to about 1 wt %, about 0.05 to about 0.5 wt %, about 1 to about 30 wt %, or about 1 to about 20 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient.

Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modern Pharmaceutics, 5^{th} Ed. (Alexander T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E. eds., Pharmaceutical Press (2012)) (each of which hereby incorporated by reference in its entirety).

The pharmaceutically acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sufate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R″′R″″Y⁻, in which the R radicals are identical or different optionally hydroxylated hydrocarbon radicals and Y⁻is an anion of a strong acid, such as halide, sulfate and sulfonate anions; cetyltrimethylammonium bromide is one of the cationic surfactants which can be used, amine salts of formula N⁺R′R″R″′, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain wetting or emulsifying agents, or pH buffering agents.

Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

### Uses of Compounds and Pharmaceutically Acceptable Compositions

Compounds and compositions described herein are generally useful for the inhibition of a kinase or a mutant thereof. In some embodiments, the kinase inhibited by the compounds and compositions described herein is a cyclin dependent kinase (CDK). In some embodiments, the kinase inhibited by the compounds and compositions described herein is one or more of CDK1, CDK2, CDK4, and CDK6. In some embodiments, the kinase inhibited by the compounds and compositions described herein is CDK2.

Compounds or compositions of the disclosure can be useful in applications that benefit from inhibition of CDK2 enzymes. For example, CDK2 inhibitors described herein are useful for the treatment of proliferative diseases generally.

CDK2 is known to be an factor in tumorigenesis and proliferation in many cancer types including lung cancer, liver cancer, colon cancer and breast cancer (Opyrchal, Int J Oncol 2014; Shi, PLoS One 2015; Lim, Cancer Prev Res 2014). There is evidence showing that CDK2 is functionally linked with hyper proliferation in multiple cancer cells and is a potential therapeutic target for cancer therapy (Chohan, Curr Med Chem 2015).

CDK2 μlays a role for the malignant transformation of breast epithelial cells. Suppression of CDK2 activity can effectively inhibit the proliferation of human breast cancer cells (Ali, Cancer Res 2009). Active CDK2 in the form of a cyclin D1/CDK2 fusion protein induces tumors that contain an invasive component that exhibits multiple features in common with human basal-like tumors and tumor-derived cell lines (Corsino, Neoplasia 2008). Cyclin D1/CDK2 complexes were detected in human breast cancer cell lines (Sweeney, Oncogene 1998), and the levels of these complexes correlated well with the degree of cyclin D1 overexpression.

The role of cyclin E and its associated kinase CDK2 in ovarian cancer has been investigated by screening primary, metastatic, recurrent and benign ovarian tumors. Using gene amplification, Cyclin E was shown to be amplified in 21% and CDK2 in 6.4% of the cases analyzed. Additionally, Cyclin E RNA was overexpressed in 29.5% and CDK2 in 6.5% of ovarian tumors tested. Cyclin E and CDK2 were overexpressed mostly in primary ovarian cancers (32% and 10%, respectively) compared to metastatic and recurrent diseases (Marone, Int J Cancer 1998).

CDK2 expression has been found to be significantly elevated in glioma tumor especially in Glioblastoma Multiforme (GBM) and was functionally required for GBM cell proliferation and tumorigenesis (Wang, Transl Oncol 2016). CDK2 expression was identified to be significantly enriched in GBM tumors and functionally required for tumor proliferation both in vitro and in vivo. Additionally, high CDK2 expression was associated to poor prognosis in GBM patients. Radio resistance is a major factor of poor clinical prognosis and tumor recurrence in GBM patients. CDK2 was found to be one of the most up-regulated kinase encoding genes in GBM after radio treatment. CDK2-dependent radio resistance is indispensable for GBM tumorigenesis and recurrence after therapeutic treatment (Id.).

Elevated levels of CDK2 expression have been observed in human cholangiocarcinoma tissues where apoptosis-related protein-1 dependent suppression of CDK2 induced cell cycle arrest and restrained tumor growth (Zheng, Oncol Rep 2016).

CDK2 overexpression in oral squamous cell carcinoma (SCC) may elevate pRB phosphorylation and permit more rapid entry of the cancer cells into S phase. In a clinicopathological survey of oral SCC, incidence of CDK2 expression was high in the poorly differentiated lesions, and was associated with the mode of tumor invasion, lymph node involvement and survival, an indication that change in CDK2 expression is associated with oral cancer progression (Mihara, Jpn J Cancer Res 2001). CDK2 expression was significantly correlated with lymph node involvement, tumor differentiation, mode of tumor invasion, and shorter survival period. Thus, increased expression of CDK2 is a factor in oral cancer progression and a negative predictive marker of the patients' prognosis (Id.).

CDK2 has been found to play a role in cell proliferation of non-small cell lung cancer (Kawana, Am J Pathol 1998). CDK2 has also been found to play a role in cell proliferation of prostate cancer (Flores, Endocrinology 2010).

The activity of a compound described herein as an inhibitor of an CDK kinase, for example, CDK2, or a mutant thereof, may be assayed in vitro, in vivo or in a cell line. In vitro assays include assays that determine inhibition of either the phosphorylation activity and/or the subsequent functional consequences, or ATPase activity of activated CDK2, or a mutant thereof. Alternative in vitro assays quantitate the ability of the inhibitor to bind to CDK2. Inhibitor binding may be measured by radiolabeling the inhibitor prior to binding, isolating the inhibitor/CDK2 complex and determining the amount of radiolabel bound. Alternatively, inhibitor binding may be determined by running a competition experiment where new inhibitors are incubated with CDK2 bound to known radioligands. Representative in vitro and in vivo assays useful in assaying an CDK2 inhibitor include those described and disclosed in the patent and scientific publications described herein. Detailed conditions for assaying a compound described herein as an inhibitor of CDK2, or a mutant thereof, are set forth in the Examples below.

Treatment of Disorders

Provided compounds are inhibitors of CDK2 and are therefore useful for treating one or more disorders associated with activity of CDK2 or mutants thereof Thus, in certain embodiments, the present disclosure provides a method of treating an CDK2-mediated disorder in a subject comprising administering a therapeutically effective amount of a compound described herein, or a pharmaceutically acceptable salt thereof, or a pharmaceutically acceptable composition of either of the foregoing, to a subject in need thereof. In certain embodiments, the present disclosure provides a method of treating an CDK2-mediated disorder in a subject comprising administering a therapeutically effective amount of a compound described herein, or a pharmaceutically acceptable composition thereof, to a subject in need thereof.

As used herein, the term “CDK2-mediated” disorders, diseases, and/or conditions means any disease or other deleterious condition in which CDK2 or a mutant thereof is known to play a role. Accordingly, another embodiment of the present disclosure relates to treating or lessening the severity of one or more diseases in which CDK2, or a mutant thereof, is known to play a role. Such CDK2-mediated disorders include but are not limited to proliferative disorders (e.g. cancer).

In some embodiments, the present disclosure provides a method for treating one or more disorders, wherein the disorders are selected from proliferative disorders and craniosynostotic syndromes, said method comprising administering to a patient in need thereof, a therapeutically effective amount of a compound of the present disclosure, or a pharmaceutically acceptable salt thereof, or a pharmaceutically acceptable composition of either of the foregoing. In some embodiments, the present disclosure provides a method for treating one or more disorders, wherein the disorders are selected from proliferative disorders and craniosynostotic syndromes, said method comprising administering to a patient in need thereof, a therapeutically effective amount of a compound of the present disclosure, or a pharmaceutically acceptable composition thereof.

In some embodiments, the disorder is associated with CDK2 signaling. CDK2 is known to have multiple upstream and downstream signaling pathways and inhibition of CDK2 can be used to treat disorders associated with aberrant signaling within those pathways. In some embodiments, the disorder is associated with cyclin E, cyclin E1, or retinoblastoma protein (R^{B}) signaling.

In some embodiments, the method of treatment comprises the steps of: i) identifying a subject in need of such treatment; (ii) providing a disclosed compound, or a pharmaceutically acceptable salt thereof, and (iii) administering said provided compound in a therapeutically effective amount to treat, suppress and/or prevent the disease state or condition in a subject in need of such treatment.

In some embodiments, the method of treatment comprises the steps of: i) identifying a subject in need of such treatment; (ii) providing a composition comprising a disclosed compound, or a pharmaceutically acceptable salt thereof; and (iii) administering said composition in a therapeutically effective amount to treat, suppress and/or prevent the disease state or condition in a subject in need of such treatment.

Another aspect of the disclosure provides a compound according to the definitions herein, or a pharmaceutically acceptable salt thereof, or a pharmaceutical composition of either of the foregoing, for use in the treatment of a disorder described herein. Another aspect of the disclosure provides the use of a compound according to the definitions herein, or a pharmaceutically acceptable salt thereof, or a pharmaceutical composition of either of the foregoing, for the treatment of a disorder described herein. Similarly, the disclosure provides the use of a compound according to the definitions herein, or a pharmaceutically acceptable salt thereof, for the preparation of a medicament for the treatment of a disorder described herein.

Proliferative Disorders

In some embodiments, the disorder is a proliferative disorder. In some embodiments, the proliferative disorder is cancer. In some embodiments, the proliferative disorder is ovarian cancer, breast cancer, lung cancer, colorectal cancer, or a combination thereof. In some embodiments, the proliferative disorder is a leukemia. In some embodiments, the proliferative disorder is breast cancer. In some embodiments, the proliferative disorder is a lung cancer. In some embodiments, the proliferative disorder is colorectal cancer.

In some embodiments, the proliferative disorder is breast cancer, prostate cancer, lung squamous cell carcinoma, thyroid cancer, gastric cancer, ovarian cancer, rectal cancer, endometrial carcinoma, non-small cell lung cancer, or bladder cancer. In some embodiments, the proliferative disorder is intrahepatic cholangiocarcinoma, hepatocellular carcinoma, breast cancer, prostate cancer, lung squamous cell carcinoma, thyroid cancer, gastric cancer, or ovarian cancer. In some embodiments, the proliferative disorder is gastric cancer, breast cancer, triple negative breast cancer, or rectal cancer. In some embodiments, the proliferative disorder is endometrial carcinoma, non-small cell lung cancer, lung squamous cell carcinoma, gastric cancer, breast cancer, or urothelial cancer.

In some embodiments, the disorder is ovarian cancer, endometrial cancer, gastric cancer, breast cancer, lung cancer, bladder cancer, cervical cancer, stomach cancer, sarcoma cancer, liver cancer, esophageal cancer, laryngeal cancer, multiple myeloma, colorectal cancer, rectal cancer, skin cancer, or pancreatic cancer. In some embodiments, the bladder cancer is urothelial carcinoma. In some embodiments, the liver cancer is hepatocellular carcinoma. In some embodiments, the lung cancer is lung squamous cell carcinoma or non-small cell lung cancer. In some embodiments, the laryngeal cancer is laryngeal squamous cell carcinoma. In some embodiments, the skin cancer is melanoma.

In some embodiments, the proliferative disorder is associated with a deregulation of CDK2 or cyclin E. In some embodiments, the deregulation of CDK2 is an overexpression of CDK2 or cyclin E. In some embodiments, the deregulation of cyclin E is an overexpression of CDK2 or cyclin E. In some embodiments, the proliferative disorder is associated with a deregulation of CDK2 and cyclin E. In some embodiments, the deregulation of CDK2 and cyclin E is an overexpression of CDK2 and cyclin E.

In some embodiments, the proliferative disorder is associated with one or more activating mutations in CDK2. In some embodiments, the activating mutation in CDK2 is a mutation to one or more of the intracellular kinase domain and the extracellular domain. In some embodiments, the activating mutation in CDK2 is a mutation to the intracellular kinase domain. Routes of Administration and Dosage Forms

The compounds and compositions, according to the methods described herein, may be administered using any amount and any route of administration effective for treating or lessening the severity of the disorder (e.g. a proliferative disorder or craniosynostotic syndrome). The exact amount required will vary from subject to subject, depending on the species, age, and general condition of the subject, the severity of the infection, the particular agent, its mode of administration, and the like. Compounds described herein are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions described herein can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds described herein may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Dosage Amounts and Regimens

In accordance with the methods of the present disclosure, the compounds of the disclosure are administered to the subject in a therapeutically effective amount, e.g., to reduce or ameliorate symptoms of the disorder in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg.

In some embodiments, the methods comprise a single dosage or administration (e.g., as a single injection or deposition). Alternatively, in some embodiments, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years or decades. In still other embodiments, the methods comprise administration over the course of several weeks. In still other embodiments, the methods comprise administration over the course of several months. In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

Inhibition of Protein Kinases

According to one embodiment, the present disclosure relates to a method of inhibiting protein kinase activity in a biological sample comprising the step of contacting said biological sample with a compound described herein, or a composition comprising said compound.

According to another embodiment, the present disclosure relates to a method of inhibiting activity of CDK2, or a mutant thereof, in a biological sample comprising the step of contacting said biological sample with a compound described herein, or a composition comprising said compound. In certain embodiments, the present disclosure relates to a method of reversibly inhibiting CDK2, or a mutant thereof, activity in a biological sample comprising the step of contacting said biological sample with a compound described herein, or a composition comprising said compound.

In another embodiment, the present disclosure provides a method of selectively inhibiting CDK2 over one or more of CDK1, CDK4, CDK5, CDK6, and CDK9. In some embodiments, a compound described herein is more than 5-fold selective over CDK1, CDK4, CDK5, CDK6, and CDK9. In some embodiments, a compound described herein is more than 10-fold selective over CDK1, CDK4, CDK5, CDK6, and CDK9. In some embodiments, a compound described herein is more than 50-fold selective over CDK1, CDK4, CDK5, CDK6, sand CDK9. In some embodiments, a compound described herein is more than 100-fold selective over CDK1, CDK4, CDK5, CDK6, and CDK9. In some embodiments, a compound described herein is more than 200-fold selective over CDK1, CDK4, CDK5, CDK6, and CDK9.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of activity of CDK2 (or a mutant thereof) in a biological sample is useful for a variety of purposes that are known to one of skill in the art. Examples of such purposes include, but are not limited to, blood transfusion, organ-transplantation, biological specimen storage, and biological assays.

Another embodiment of the present disclosure relates to a method of inhibiting protein kinase activity in a patient comprising the step of administering to said patient a compound described herein, or a composition comprising said compound.

According to another embodiment, the present disclosure relates to a method of inhibiting activity of CDK2, or a mutant thereof, in a patient comprising the step of administering to said patient a compound described herein, or a composition comprising said compound. According to certain embodiments, the present disclosure relates to a method of reversibly inhibiting activity of one or more of CDK2, or a mutant thereof, in a patient comprising the step of administering to said patient a compound described herein, or a composition comprising said compound.

According to another embodiment, the present disclosure provides a method for treating a disorder mediated by CDK2, or a mutant thereof, in a patient in need thereof, comprising the step of administering to said patient a compound described herein or a pharmaceutically acceptable composition thereof. Such disorders are described in detail herein. In some embodiments, the present disclosure provides a method for treating a disorder mediated by CDK2, or a mutant thereof, in a patient in need thereof, comprising the step of administering to said patient a compound described herein or a pharmaceutically acceptable composition thereof, wherein the compound reversibly inhibits the CDK2, or a mutant thereof.

According to another embodiment, the present disclosure provides a method of inhibiting signaling activity of CDK2, or a mutant thereof, in a subject, comprising administering a therapeutically effective amount of a compound described herein, or a pharmaceutically acceptable composition thereof, to a subject in need thereof. In some embodiments, the present disclosure provides a method of inhibiting CDK2 signaling activity in a subject, comprising administering a therapeutically effective amount of a compound described herein, or a pharmaceutically acceptable composition thereof, to a subject in need thereof.

In some embodiments, the present disclosure provides a method for treating a disorder mediated by CDK2, or a mutant thereof, in a patient in need thereof, comprising the step of administering to said patient a compound described herein or a pharmaceutically acceptable composition thereof, wherein the compound reversibly inhibits the CDK2, or a mutant thereof.

The compounds described herein can also inhibit CDK2 function through incorporation into agents that catalyze the destruction of CDK2. For example, the compounds can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. The portion of the PROTAC that engages the E3 ligase is connected to the portion of the PROTAC that engages the target protein via a linker which consists of a variable chain of atoms. Recruitment of CDK2 to the E3 ligase will thus result in the destruction of the CDK2 protein. The variable chain of atoms can include, for example, rings, heteroatoms, and/or repeating polymeric units. It can be rigid or flexible. It can be attached to the two portions described above using standard techniques in the art of organic synthesis.

Combination Therapies

Depending upon the particular disorder, condition, or disease, to be treated, additional therapeutic agents, that are normally administered to treat that condition, may be administered in combination with compounds and compositions described herein. As used herein, additional therapeutic agents that are normally administered to treat a particular disease, or condition, are known as “appropriate for the disease, or condition, being treated.”

Accordingly, in certain embodiments, the method of treatment comprises administering the compound or composition described herein in combination with one or more additional therapeutic agents. In certain other embodiments, the methods of treatment comprise administering the compound or composition described herein as the only therapeutic agent.

In some embodiments, the one or more additional therapeutic agents is selected from antibodies, antibody-drug conjugates, kinase inhibitors, immunomodulators, and histone deacetylase inhibitors. In some embodiments, the one or more additional therapeutic agent is selected from the following agents, or a pharmaceutically acceptable salt thereof: BCR-ABL inhibitors: e.g. imatinib, inilotinib, nilotinib, dasatinib, bosutinib, ponatinib, bafetinib, danusertib, saracatinib, PF03814735; ALK inhibitors (see Dardaei et al, 2018, Nat Med.; 24(4):512-517): e.g. crizotinib, NVP-TAE684, ceritinib, alectinib, brigatinib, entrecinib, lorlatinib; BRAF inhibitors (see Prahallad et al, 2015, Cell Rep. 12, 1978-1985): e.g. vemurafenib, dabrafenib; FGFR inhibitors: e.g. infigratinib, dovitinib, erdafitinib, BLU-554, AZD4547; FLT3 inhibitors: e.g. sunitinib, midostaurin, tanutinib, sorafenib, lestaurtinib, quizartinib, and crenolanib; MEK Inhibitors (see Fedele et al, 2018, BioRxiv 307876; Torres-Ayuso et al, 2018, Cancer Discov. 8, 1210-1212; and Wong et al, 2016, Oncotarget. 2016 Oct. 4; 7(40): 65676-65695): e.g. trametinib, cobimetinib, binimetinib, selumetinib; ERK inhibitors: e.g. ulixertinib, MK-8353, LY-3214996; VEGF receptor inhibitors: e.g. bevacizumab, axitinib, aflibercept, brivanib, motesanib, pasireotide, sorafenib; Tyrosine kinase inhibitors: e.g. erlotinib, linifanib, sunitinib, pazopanib; Epidermal growth factor receptor (EGFR) inhibitors: gefitnib, osimertinib, cetuximab, panitumumab; HER2 receptor inhibitors: e.g. trastuzumab, neratinib, lapatinib, lapatinib; MET inhibitors: e.g. crizotinib, cabozantinib; CD20 antibodies: e.g. rituximab, tositumomab, ofatumumab; DNA Synthesis inhibitors: e.g. capecitabine, gemcitabine, nelarabine, hydroxycarbamide; Antineoplastic agents: e.g. oxaliplatin, cisplatin; HER dimerization inhibitors: e.g. pertuzumab; Human Granulocyte colony-stimulating factor (G-CSF) modulators: e.g. filgrastim; Immunomodulators: e.g. afutuzumab, lenalidomide, thalidomide, pomalidomide; CD40 inhibitors: e.g. dacetuzumab; Pro-apoptotic receptor agonists (PARAs): e.g. dulanermin; Heat Shock Protein (HSP) inhibitors: e.g. tanespimycin (17-allylamino-17-desmethoxygeldanamycin); Hedgehog antagonists: e.g. vismodegib; Proteasome inhibitors: e.g. bortezomib; PI3K inhibitors: e.g. pictilisib, dactolisib, buparlisib, taselisib, idelalisib, duvelisib, umbralisib; Phospholipase A2 inhibitors: e.g. anagrelide; BCL-2 inhibitors: e.g. venetoclax; Aromatase inhibitors: exemestane, letrozole, anastrozole, faslodex, tamoxifen; Topoisomerase I inhibitors: e.g. irinotecan, topotecan; Topoisomerase II inhibitors: e.g. etoposide, teniposide; mTOR inhibitors: e.g. temsirolimus, ridaforolimus, everolimus, sirolimus; Osteoclastic bone resorption inhibitors: e.g. zoledronic acid; CD33 Antibody Drug Conjugates: e.g. gemtuzumab ozogamicin; CD22 Antibody Drug Conjugates: e.g. inotuzumab ozogamicin; CD20 Antibody Drug Conjugates: e.g. ibritumomab tiuxetan; Somatostain analogs: e.g. octreotide; Interleukin-11 (IL-11): e.g. oprelvekin; Synthetic erythropoietin: e.g. darbepoetin alfa; Receptor Activator for Nuclear Factor κ B (RANK) inhibitors: e.g. denosumab; Thrombopoietin mimetic peptides: e.g. romiplostim; Cell growth stimulators: e.g. palifermin; Anti-Insulin-like Growth Factor-1 receptor (IGF-1R) antibodies: e.g. figitumumab; Anti-CSl antibodies: e.g. elotuzumab; CD52 antibodies: e.g. alemtuzumab; CTLA-4 inhibitors: e.g. tremelimumab, ipilimumab; PD1 inhibitors: e.g. nivolumab, pembrolizumab; an immunoadhesin; e.g. pidilizumab, AMP-224; PDL1 inhibitors: e.g. MSB0010718C; YW243.55.S70, MPDL3280A; MEDI-4736, MSB-0010718C, or MDX-1105; LAG-3 inhibitors: e.g. BMS-986016; GITR agonists; GITR fusion proteins and anti-GITR antibodies; Histone deacetylase inhibitors (HDI): e.g. voninostat; Anti-CTLA4 antibodies: e.g. tremelimumab, ipilimumab; Alkylating agents: e.g. temozolomide, dactinomycin, melphalan, altretamine carmustine, bendamustine, busulfan, carboplatin, lomustine, cisplatin, chlorambucil, cyclophosphamide, dacarbazine, altretamine, ifosfamide, procarbazine, mechlorethamine, mustine and mechloroethamine, streptozocin, thiotepa; Biologic response modifiers: e.g. bacillus calmette-guerin, denileukin diftitox; Anti-tumor antibiotics: e.g. doxorubicin, bleomycin, daunorubicin, daunorubicin liposomal, mitoxantrone, epirubicin, idarubicin, mitomycin C; Anti-microtubule agents: e.g. estramustine; Cathepsin K inhibitors: e.g. odanacatib; Epothilone analogs: e.g. ixabepilone; TpoR agonists: e.g. eltrombopag; Anti-mitotic agents: e.g. docetaxel; Adrenal steroid inhibitors: e.g. aminoglutethimide; Anti-androgens: e.g. nilutamide; Androgen Receptor inhibitors: e.g. enzalutamide, abiraterone acetate, orteronel, galeterone, and seviteronel, bicalutamide, flutamide; Androgens: e.g. fluoxymesterone; CDK1 inhibitors: e.g. alvocidib, palbociclib, ribociclib, trilaciclib, abemaciclib; Gonadotropin-releasing hormone (GnRH) receptor agonists: e.g. leuprolide or leuprolide acetate; Taxane anti-neoplastic agents: e.g. cabazitaxel, larotaxel; 5-HTla receptor agonists: e.g. xaliproden; HPV vaccines: e.g. Cervarix® sold by GlaxoSmithKline, Gardasil® sold by Merck; Iron Chelating agents: e.g. deferasirox; Anti-metabolites: e.g. claribine, 5-fluorouracil, 6-thioguanine, pemetrexed, cytarabine, cytarabine liposomal, decitabine, hydroxyurea, fludarabine, floxuridine, cladribine, methotrexate, pentostatin; Bisphosphonates: e.g. pamidronate; Demethylating agents: e.g. 5-azacitidine, decitabine; Anti-tumor Plant Alkaloids: e.g. paclitaxel protein-bound; vinblastine, vincristine, vinorelbine, paclitaxel; Retinoids: e.g. alitretinoin, tretinoin, isotretinoin, bexarotene; Glucocorticosteroids: e.g. hydrocortisone, dexamethasone, prednisolone, prednisone, methylprednisolone; Cytokines: e.g. interleukin-2, interleukin-11 (oprevelkin), alpha interferon alfa (IFN-alpha); estrogen receptor downregulators: fulvestrant; Anti-estrogens: e.g. tamoxifen, toremifene; Selective estrogen receptor modulators (SERMs): e.g. raloxifene; Luteinizing hormone releasing hormone (LHRH) agonists: e.g. goserelin; Progesterones: e.g. megestrol; cytotoxic agents: arsenic trioxide, asparaginase (also known as L-asparaginase, Erwinia L-asparaginase; Anti-nausea drugs: e.g. NK-1 receptor antagonists (e.g. casopitant); Cytoprotective agents: e.g. amifostine, leucovorin; and Immune checkpoint inhibitors. The term “immune checkpoints” refers to a group of molecules on the cell surface of CD4 and CD8 T cells. Immune checkpoint molecules include, but are not limited to, Programmed Death 1 (PD-1), Cytotoxic T-Lymphocyte Antigen 4 (CTLA-4), B7H1, B7H4, OX-40, CD 137, CD40, and LAG3. Immunotherapeutic agents which can act as immune checkpoint inhibitors useful in the methods of the present disclosure, include, but are not limited to, inhibitors of PD-L1, PD-L2, CTLA4, TIM3, LAG3, VISTA, BTLA, TIGIT, LAIR1, CD 160, 2B4 and/or TGFR beta.

In some embodiments, the one or more additional therapeutic agent is selected from the following agents: anti-CDK2 antibodies; cytotoxic agents; Estrogen Receptor-targeted or other endocrine therapies, immune-checkpoint inhibitors, other CDK inhibitors, Receptor Tyrosine Kinase inhibitors, BRAF inhibitors, MEK inhibitors, PI3K inhibitors, SHP2 inhibitors, and SRC inhibitors. (See M. Katoh, Nat. Rev. Clin. Oncol. 2019, 16:105-122; Y. K. Chae, et al. Oncotarget 2017, 8:16052-16074; L. Formisano et al., Nat. Comm. 2019, 10:1373-1386; and references cited therein.)

The structure of the active compounds identified by code numbers, generic or trade names may be taken from the actual edition of the standard compendium “The Merck Index” or from databases, e.g. Patents International (e.g. IMS World Publications).

A compound described herein may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound described herein can be administered alone or in combination with one or more other therapeutic compounds, possible combination therapy taking the form of fixed combinations or the administration of a compound described herein and one or more other therapeutic compounds being staggered or given independently of one another, or the combined administration of fixed combinations and one or more other therapeutic compounds. A compound described herein can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from a provided compound-containing composition, as part of a multiple dosage regimen. Alternatively, those agents may be part of a single dosage form, mixed together with a compound described herein in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this disclosure. For example, a compound described herein may be administered with another therapeutic agent simultaneously or sequentially in separate unit dosage forms or together in a single unit dosage form. Accordingly, the present disclosure provides a single unit dosage form comprising a compound described herein, an additional therapeutic agent, and a pharmaceutically acceptable carrier, adjuvant, or vehicle.

The amount of both a compound described herein and additional therapeutic agent (in those compositions which comprise an additional therapeutic agent as described above) that may be combined with the carrier materials to produce a single dosage form will vary depending upon the host treated and the particular mode of administration. Preferably, compositions described herein should be formulated so that a dosage of between 0.01-100 mg/kg body weight/day of a compound described herein can be administered.

In those compositions which comprise an additional therapeutic agent, that additional therapeutic agent and the compound described herein may act synergistically. Therefore, the amount of additional therapeutic agent in such compositions will be less than that required in a monotherapy utilizing only that therapeutic agent. In such compositions a dosage of between 0.01-1,000 pg/kg body weight/day of the additional therapeutic agent can be administered.

The amount of additional therapeutic agent present in the compositions described herein will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds described herein, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. The present disclosure also contemplates implantable devices coated with a compound described herein.

Any of the compounds and/or compositions of the disclosure may be provided in a kit comprising the compounds and/or compositions. Thus, in some embodiments, the compound and/or composition of the disclosure is provided in a kit.

The disclosure is further described by the following non-limiting Examples.

### EXAMPLES

Examples are provided herein to facilitate a more complete understanding of the disclosure. The following examples serve to illustrate the exemplary modes of making and practicing the subject matter of the disclosure. However, the scope of the disclosure is not to be construed as limited to specific embodiments disclosed in these examples, which are illustrative only.

As depicted in the Examples below, in certain exemplary embodiments, compounds are prepared according to the following general procedures. It will be appreciated that, although the general methods depict the synthesis of certain compounds described herein, the following general methods, and other methods known to one of ordinary skill in the art, can be applied to other classes and subclasses and species of each of these compounds, as described herein. Additional compounds described herein were prepared by methods substantially similar to those described herein in the Examples and methods known to one skilled in the art.

In the description of the synthetic methods described below, unless otherwise stated, it is to be understood that all reaction conditions (for example, reaction solvent, atmosphere, temperature, duration, and workup procedures) are selected from the standard conditions for that reaction, unless otherwise indicated. In the general schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). The starting materials for the examples are either commercially available or are readily prepared by standard methods from known materials.

At least some of the compounds identified as “Intermediates” herein are contemplated as compounds of the disclosure.

### Example 1

### N-(1-methylcyclopropyl)-2-((1R,3S)-3-(3-(2-(3-methylisoxazol-5-yl)acetamido)-1H-pyrazol-5-yl)cyclopentyl)acetamide

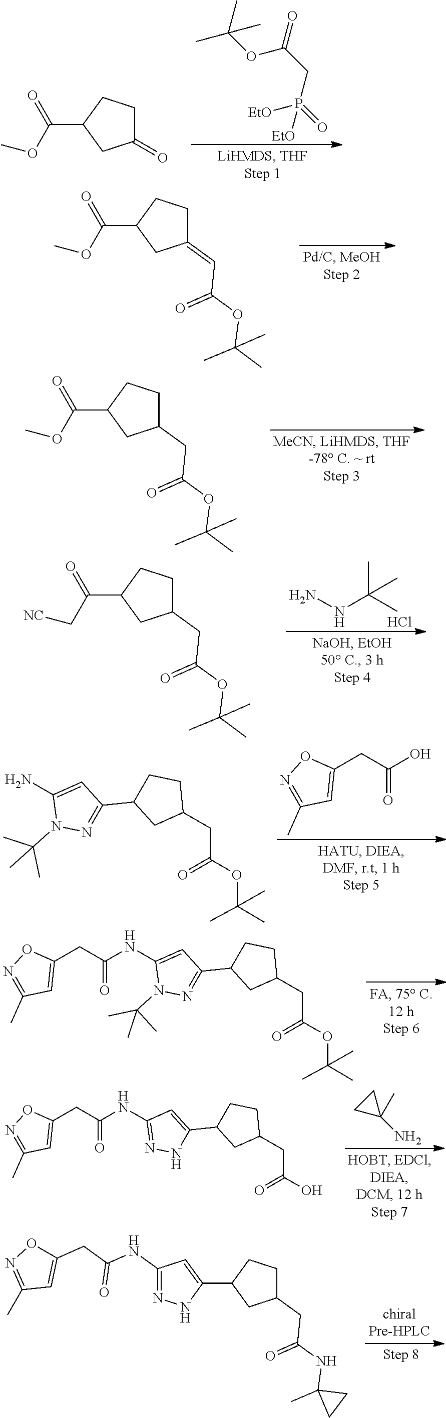

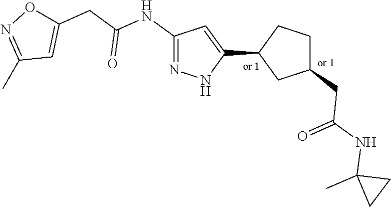 [SMILES: Cc1cc(CC(=O)Nc2cc([C@H]3CC[C@@H](CC(=O)NC4(C)CC4)C3)[nH]n2)on1]

### Methyl (Z)-3-(2-(tert-butoxy)-2-oxoethylidene)cyclopentane-1-carboxylate

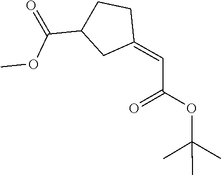 [SMILES: COC(=O)C1CC/C(=C/C(=O)OC(C)(C)C)C1]

Step 1: To a mixture of tert-butyl 2-(diethoxyphosphoryl)acetate (35 g, 0.14 mol) in THE (100 mL) was added LiHMDS (140 mL, 0.14 mol) dropwise at 0° C. under nitrogen atmosphere. The mixture was stirred for 1 h at 0° C. prior to the addition of methyl 3-oxocyclopentane-1-carboxylate (10 g, 70 mmol). The mixture was stirred for 2 h at 25° C. The reaction mixture was quenched with saturated NH₄C₁ (50 mL), and the aqueous phase was extracted with ethyl acetate (100 mL) three times. The combined organic layers were washed with brine, dried over sodium sulfate, filtered, and concentrated in vacuo. The resulting crude material was purified by silica gel chromatography (20 g column; eluting with PE/EA; 8/1). Concentration in vacuo resulted in methyl (Z)-3-(2-(tert-butoxy)-2-oxoethylidene)cyclopentane-1-carboxylate (4.8 g, 28%) as a colorless oil. m/z (ES⁺) [M+H]⁺=241.10; HPLC tR=1.227 min.

### Methyl 3-(2-(tert-butoxy)-2-oxoethyl)cyclopentane-1-carboxylate

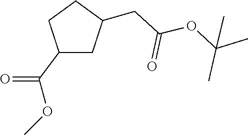 [SMILES: COC(=O)C1CCC(CC(=O)OC(C)(C)C)C1]

Step 2: A stirred mixture of methyl (Z)-3-(2-(tert-butoxy)-2-oxoethylidene)cyclopentane-1-carboxylate (4.8 g, 20 mmol) and Pd/C (0.43 g, 4.0 mmol) in MeOH (50 mL) was treated with H₂ for 2 h at 25° C. The mixture was filtered through a Celite pad. The filtrate was concentrated under vacuum. Concentration in vacuo resulted in methyl 3-(2-(tert-butoxy)-2-oxoethyl)cyclopentane-1-carboxylate (4.1 g, 85%) as a colorless oil. m/z (ES⁺) [M+Na]=265.15; HPLC tR=1.243 min.

### tert-butyl 2-(3-(2-cyanoacetyl)cyclopentyl)acetate

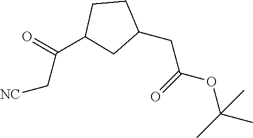 [SMILES: CC(C)(C)OC(=O)CC1CCC(C(=O)CC#N)C1]

Step 3: To a mixture of CH₃CN (1.4 g, 34 mmol) and methyl 3-(2-(tert-butoxy)-2-oxoethyl)cyclopentane-1-carboxylate (4.1 g, 17 mmol) in THE (40 mL) was added LiHMDS (25 mL, 25 mmol) dropwise at −78° C. under nitrogen atmosphere. The mixture was warmed to room temperature slowly and stirred for another 2 h at room temperature. The reaction mixture was quenched with H₂O (50 mL), and the aqueous phase was extracted with EA (100 mL) three times. The combined organic layers were washed with brine, dried over sodium sulfate, filtered, and concentrated in vacuo. tert-butyl 2-(3-(2-cyanoacetyl)cyclopentyl)acetate (1.6 g, 38%) was isolated as a colorless oil. m/z (ES⁺) [M+H]⁺=252.10; HPLC tR=0.982 min.

### tert-butyl 2-(3-(5-amino-1-(tert-butyl)-1H-pyrazol-3-yl)cyclopentyl)acetate

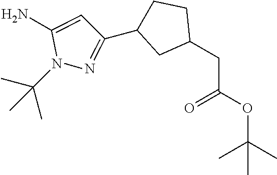 [SMILES: CC(C)(C)OC(=O)CC1CCC(c2cc(N)n(C(C)(C)C)n2)C1]

Step 4: To a mixture of tert-butylhydrazine (0.88 g, 9.9 mmol) in EtOH (5 mL) was added NaOH (0.40 g, 9.9 mmol) in portions at 25° C. under nitrogen atmosphere. The mixture was stirred for 1 h at 25° C. prior to the addition of tert-butyl 2-(3-(2-cyanoacetyl)cyclopentyl)acetate (2.5 g, 9.9 mmol). The mixture was stirred for 3 h at 50° C. The reaction mixture was diluted with H₂O (50 mL), and extracted with EA (100 mL) three times. The combined organic layers were washed with brine, dried over sodium sulfate, filtered, and concentrated in vacuo. The residue was purified by reverse flash chromatography with the following conditions: column, C₁₈ silica gel; mobile phase, MeCN in water, 0% to 100% gradient in 15 min; detector, UV 220 nm. Concentration in vacuo resulted in tert-butyl 2-(3-(5-amino-1-(tert-butyl)-1H-pyrazol-3-yl)cyclopentyl)acetate (2 g, 60%) as a colorless oil. m/z (ES⁺) [M+H]⁺=322.30; HPLC tR=0.985 min. tert-butyl 2-(3-(1-(tert-butyl)-5-(2-(3-methylisoxazol-5-yl)acetamido)-1H-pyrazol-3-yl)cyclopentyl)acetate

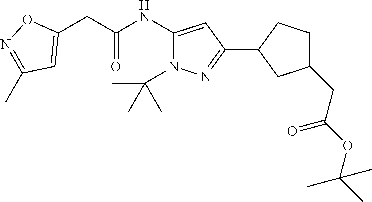 [SMILES: Cc1cc(CC(=O)Nc2cc(C3CCC(CC(=O)OC(C)(C)C)C3)nn2C(C)(C)C)on1]

Step 5: A round bottomed flask was charged with tert-butyl 2-(3-(5-amino-1-(tert-butyl)-1H-pyrazol-3-yl)cyclopentyl)acetate (2 g, 6 mmol), 2-(3-methylisoxazol-5-yl)acetic acid (1 g, 7 mmol), HATU (4 g, 9 mmol), DIEA (2 g, 0.02 mol), and DCM (20 mL). The solution was stirred for 1 h at 25° C. The reaction mixture was diluted with H₂O (50 mL), and the aqueous phase was extracted with EA (50 mL) three times. The combined organic layers were washed with brine, dried over sodium sulfate, filtered, and concentrated in vacuo. The resulting crude material was purified by silica gel chromatography eluting with DCM/MeOH=20/1. Concentration in vacuo resulted in tert-butyl 2-(3-(1-(tert-butyl)-5-(2-(3-methylisoxazol-5-yl)acetamido)-1H-pyrazol-3-yl)cyclopentyl)acetate (800 mg, 30%) as a colorless oil. m/z (ES⁺) [M+H]⁺=445.35; HPLC tR=1.243 min.

### 2-(3-(3-(2-(3-methylisoxazol-5-yl)acetamido)-1H-pyrazol-5-yl)cyclopentyl)acetic acid

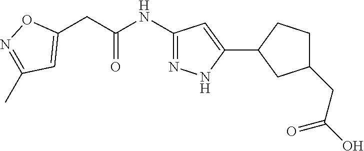 [SMILES: Cc1cc(CC(=O)Nc2cc(C3CCC(CC(=O)O)C3)[nH]n2)on1]

Step 6: A stirred mixture of tert-butyl 2-(3-(1-(tert-butyl)-5-(2-(3-methylisoxazol-5-yl)acetamido)-1H-pyrazol-3-yl)cyclopentyl)acetate (844 mg, 1.90 mmol) in FA (8 mL) was treated with N₂ for 12 h at 75° C. The resulting crude material was purified by C18 (acetonitrile/water). Lyophilization yielded 2-(3-(3-(2-(3-methylisoxazol-5-yl)acetamido)-1H-pyrazol-5-yl)cyclopentyl)acetic acid (600 mg, 95.1%) as a yellow oil. m/z (ES⁺) [M+H]+=333.20; HPLC tR=0.302 min.

### N-(1-methylcyclopropyl)-2-(3-(3-(2-(3-methylisoxazol-5-yl)acetamido)-1H-pyrazol-5-yl)cyclopentyl)acetamide

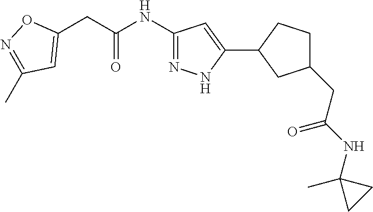 [SMILES: Cc1cc(CC(=O)Nc2cc(C3CCC(CC(=O)NC4(C)CC4)C3)[nH]n2)on1]

Step 7: A resealable reaction vial was charged with 2-(3-(3-(2-(3-methylisoxazol-5-yl)acetamido)-1H-pyrazol-5-yl)cyclopentyl)acetic acid (600 mg, 1.81 mmol), 1-methylcyclopropan-1-amine (642 mg, 9.03 mmol), HOBt (415 mg, 2.71 mmol), EDC (692 mg, 3.61 mmol), and a stirbar before being evacuated and purged with nitrogen three times. DCM (10 mL) was added, and the mixture was stirred for 1 h at 25° C. The reaction mixture was diluted with H₂O (50 mL), and the aqueous phase was extracted with EA (50 mL) three times. The combined organic layers were washed with brine, dried over sodium sulfate, filtered, and concentrated in vacuo. The resulting crude material was purified by silica gel chromatography (1 g column; eluting with DCM/MeOH; 20/1). Concentration in vacuo resulted in N-(1-methylcyclopropyl)-2-(3-(3-(2-(3-methylisoxazol-5-yl)acetamido)-1H-pyrazol-5-yl)cyclopentyl)acetamide (187 mg, 26.9%) as a colorless oil. m/z (ES⁺) [M+H]⁺=386.35; HPLC tR=0.778 min.

### N-(1-methylcyclopropyl)-2-((1R,3S)-3-(3-(2-(3-methylisoxazol-5-yl)acetamido)-1H-pyrazol-5-yl)cyclopentyl)acetamide

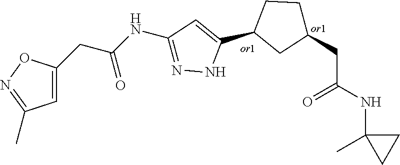 [SMILES: Cc1cc(CC(=O)Nc2cc([C@H]3CC[C@@H](CC(=O)NC4(C)CC4)C3)[nH]n2)on1]

Step 8: N-(1-methylcyclopropyl)-2-(3-(3-(2-(3-methylisoxazol-5-yl)acetamido)-1H-pyrazol-5-yl)cyclopentyl) acetamide (400 mg, 1.04 mmol) was purified by chiral Pre-HPLC (Column: CHIRALPAK IG, 2*25 cm, 5 m; Mobile Phase A: MtBE(0.1% TEA)—HPLC, Mobile Phase B: IPA: DCM=1:1; Flow rate: 20 mL/min; Gradient: 40% B to 40% B in 19 min; Wave Length: 220/254 nm; RT1(min): 11.69; RT2(min): 15.11; Sample Solvent: IPA: DCM=1:1; Injection Volume: 0.3 mL; Number Of Runs: 10). Lyophilization yielded N-(1-methylcyclopropyl)-2-((1R,3S)-3-(3-(2-(3-methylisoxazol-5-yl)acetamido)-1H-pyrazol-5-yl)cyclopentyl)acetamide (44 mg, 11%) as a white amorphous solid. m/z (ES⁺) [M+H]⁺=386.25; HPLC tR=0.800 min. ¹H NMR (400 MHz, DMSO-d₆) δ 12.07 (s, 1H), 10.61 (s, 1H), 7.99 (s, 1H), 6.24 (d, J=14.6 Hz, 2H), 3.83 (s, 2H), 3.02 (dq, J=10.7, 8.4 Hz, 1H), 2.20 (s, 4H), 2.10 (dt, J=13.5, 7.0 Hz, 1H), 2.06-1.92 (m, 3H), 1.77 (dq, J=14.8, 7.7 Hz, 1H), 1.59 (dtd, J=12.5, 9.3, 6.5 Hz, 1H), 1.31 (dtd, J=12.9, 9.1, 5.9 Hz, 1H), 1.25 (s, 3H), 1.18 (dt, J=12.3, 10.5 Hz, 1H), 0.59-0.52 (m, 2H), 0.52-0.44 (m, 2H).

Additional compounds prepared according to the methods of Example 1 are depicted in Table 2 below.

TABLE 2
Additional Exemplary Compounds

| | | | MS |
|---|---|---|---|
| Compound | Structure | Proton NMR | [M + 1] |
| N-(1- methylcyclo- propyl)- 2-((1S,3R)-3- (3-(2-(3- methylisoxazol- 5-yl)acetamido)- 1H-pyrazol-5- yl)cyclopentyl) | 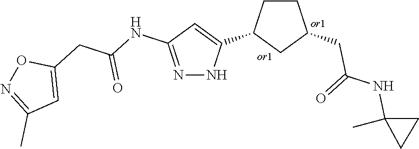 [SMILES: Cc1cc(CC(=O)Nc2cc([C@@H]3CC[C@H](CC(=O)NC4(C)CC4)C3)[nH]n2)on1] | ¹H NMR (400 MHz, DMSO-d₆) δ 12.06 (s, 1H), 10.60 (s, 1H), 7.99 (s, 1H), 6.24 (d, J = 14.7 Hz, 2H), 3.83 (s, 2H), 3.01 (dq, J = 10.7, 8.4 Hz, 1H), 2.20 (s, 4H), 2.11 (dt, J = 13.6, 7.0 Hz, 1H), 2.08-1.99 (m, 2H), 2.02-1.91 (m, 1H), 1.77 (dq, J = 14.8, 7.6 Hz, 1H), 1.59 (dtd, J = 12.4, 9.3, 6.4 Hz, 1H), 1.31 (dtd, J = | 386.2 |
| acetamide | | 12.9, 9.1, 6.0 Hz, 1H), 1.25 (s, 3H), | |
| | | 1.18 (dt, J = 12.3, 10.6 Hz, 1H), | |
| | | 0.59-0.51 (m, 2H), 0.53-0.44 (m, | |
| | | 2H). | |
| N-(1- methylcyclo- propyl)- 2-((1S,3S)-3- (3-(2-(3- methylisoxazol- 5-yl)acetamido)- 1H-pyrazol-5- yl)cyclopentyl) | 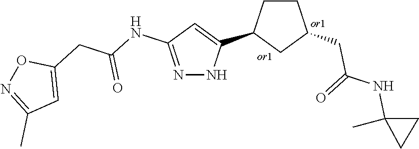 [SMILES: Cc1cc(CC(=O)Nc2cc([C@H]3CC[C@H](CC(=O)NC4(C)CC4)C3)[nH]n2)on1] | ¹H NMR (400 MHz, DMSO-d₆) δ 12.06 (s, 1H), 10.60 (s, 1H), 7.98 (s, 1H), 6.24 (d, J = 11.5 Hz, 2H), 3.83 (s, 2H), 3.11 (p, J = 7.9 Hz, 1H), 2.34 (p, J = 7.5 Hz, 1H), 2.20 (s, 3H), 2.01 (t, J = 6.5 Hz, 3H), 1.84 (dtd, J = 11.2, 7.5, 3.3 Hz, 1H), 1.74 (dt, J = 12.7, 7.8 Hz, 1H), 1.66-1.50 (m, 2H), 1.25 (s, 4H), | 386.2 |
| acetamide | | 0.61-0.51 (m, 2H), 0.53-0.44 | |
| | | (m, 2H). | |
| N-(1- methylcyclo- propyl)- 2-((1R,3R)-3- (3-(2-(3- methylisoxazol- 5-yl)acetamido)- 1H-pyrazol-5- yl)cyclopentyl) | 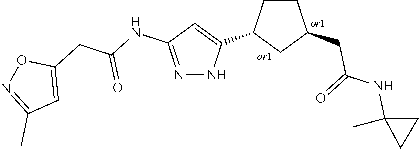 [SMILES: Cc1cc(CC(=O)Nc2cc([C@@H]3CC[C@@H](CC(=O)NC4(C)CC4)C3)[nH]n2)on1] | ¹H NMR (400 MHz, DMSO-d₆) δ 12.07 (s, 1H), 10.61 (s, 1H), 7.99 (s, 1H), 6.23 (d, J = 8.7 Hz, 2H), 3.83 (s, 2H), 3.11 (p, J = 7.9 Hz, 1H), 2.34 (p, J = 7.5 Hz, 1H), 2.20 (s, 3H), 2.01 (t, J = 6.3 Hz, 3H), 1.84 (dtd, J = 11.1, 7.5, 3.3 Hz, 1H), 1.74 (dt, J = 12.8, 7.8 Hz, 1H), 1.68- 1.49 (m, 2H), 1.25 (s, 4H), 0.62- | 386.2 |
| acetamide | | 0.51 (m, 2H), 0.53-0.44 (m, 2H). | |
| 2-((1R,3S)-3-(3- (2-(3- methylisoxazol- 5-yl)acetamido)- 1H-pyrazol-5- yl)cyclopentyl) acetic acid Compound 341 | 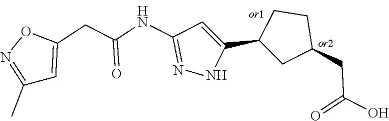 [SMILES: Cc1cc(CC(=O)Nc2cc([C@H]3CC[C@@H](CC(=O)O)C3)[nH]n2)on1] | ¹H NMR (400 MHz, DMSO-d6) 11.95 (s, 2H), 10.62 (s, 1H), 6.24 (d, J = 10.4 Hz, 2H), 3.83 (s, 2H), 3.14 (p, J = 7.9 Hz, 1H), 2.35 (dt, J = 14.9, 7.4 Hz, 1H), 2.26 (d, J = 7.4 Hz, 2H), 2.20 (s, 3H), 2.03 (dtd, J = 11.2, 7.6, 3.2 Hz, 1H), 1.92 (ddd, J = 10.8, 8.7, 4.7 Hz, | 333.15 |
| | | 1H), 1.81 (dt, J = 12.8, | |
| | | 7.6 Hz, 1H), 1.71-1.51 (m, | |
| | | 2H), 1.31-1.20 (m, 1H). | |
| 2-((1S,3R)-3-(3- (2-(3- methylisoxazol- 5-yl)acetamido)- 1H-pyrazol-5- yl)cyclopentyl) acetic acid Compound 342 | 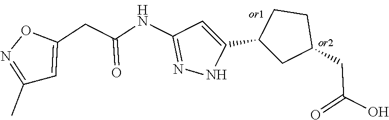 [SMILES: Cc1cc(CC(=O)Nc2cc([C@@H]3CC[C@H](CC(=O)O)C3)[nH]n2)on1] | ¹H NMR (400 MHz, DMSO-d6) 11.97 (s, 2H), 10.61 (s, 1H), 6.24 (d, J = 10.4 Hz, 2H), 3.83 (s, 2H), 3.14 (p, J = 7.9 Hz, 1H), 2.35 (dt, J = 15.0, 7.4 Hz, 1H), 2.26 (d, J = 7.3 Hz, 2H), 2.20 (s, 3H), 2.04 (ddd, J = 15.4, 10.8, 6.8 Hz, 1H), 1.98-1.87 (m, 1H), 1.87-1.76 (m, | 333.10 |
| | | 1H), 1.61 (ddt, J = 29.4, 12.5, 8.1 | |
| | | Hz, 2H), 1.26 (dt, J = 12.2, 8.4 | |
| | | Hz, 1H). | |
| 2-((1R,3R)-3-(3- (2-(3- methylisoxazol- 5-yl)acetamido)- 1H-pyrazol-5- yl)cyclopentyl) acetic acid Compound 344 | 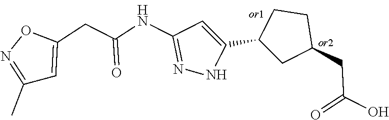 [SMILES: Cc1cc(CC(=O)Nc2cc([C@@H]3CC[C@@H](CC(=O)O)C3)[nH]n2)on1] | ¹H NMR (400 MHz, DMSO-d6) 12.02 (s, 2H), 10.61 (s, 1H), 6.24 (d, J = 13.8 Hz, 2H), 3.83 (s, 2H), 3.11-2.98 (m, 1H), 2.29 (d, J = 3.1 Hz, 3H), 2.20 (s, 4H), 1.98 (ddd, J = 13.4, 10.8, 6.8 Hz, 1H), 1.86 (dq, J = 14.1, 7.1 Hz, 1H), 1.62 (dtd, J = 12.3, 9.3, 6.4 Hz, 1H), | 333.15 |
| | | 1.42-1.28 (m, 1H), 1.22 (td, J = | |
| | | 11.3, 8.8 Hz, 1H). | |
| 2-((1S,3S)-3-(3- (2-(3- methylisoxazol- 5-yl)acetamido)- 1H-pyrazol-5- yl)cyclopentyl) acetic acid | 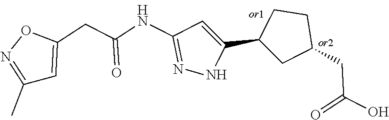 [SMILES: Cc1cc(CC(=O)Nc2cc([C@H]3CC[C@H](CC(=O)O)C3)[nH]n2)on1] | ¹H NMR (400 MHz, DMSO-d6) 11.97 (s, 2H), 10.61 (s, 1H), 6.24 (d, J = 13.1 Hz, 2H), 3.83 (s, 2H), 3.04 (p, J = 8.5 Hz, 1H), 2.31-2.26 (m, 3H), 2.20 (s, 4H), 2.05-1.92 (m, 1H), 1.87 (dt, J = 13.2, 7.0 Hz, 1H), 1.69-1.55 (m, 1H), 1.42- | 333.15 |
| | | 1.31 (m, 1H), 1.24 (t, J = 9.9 Hz, | |
| | | 1H). | |
| N-isopropyl-2- ((1s,3s)-3- (3-(2-(3- methylisoxazol- 5- yl)acetamido)- 1H-pyrazol-5- yl)cyclobutyl) acetamide | 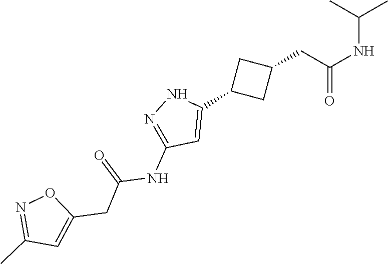 [SMILES: Cc1cc(CC(=O)Nc2cc([C@H]3C[C@@H](CC(=O)NC(C)C)C3)[nH]n2)on1] | ¹H NMR (400 MHz, DMSO-d₆) 12.08 (s, 1H), 10.63 (s, 1H), 7.62 (d, J = 7.7 Hz, 1H), 6.31 (s, 1H), 6.22 (s, 1H), 3.90-3.67 (m, 3H), 3.32-3.18 (m, 1H), 2.50-2.42 (m, 1H), 2.37 (qd, J = 8.0, 2.7 Hz, 2H), 2.21 (s, 3H), 2.15 (d, J = 7.3 Hz, 2H), 1.78 (dt, J = 12.1, 9.6 Hz, 2H), 1.02 (d, J = 6.6 Hz, 6H). | 360.15 |
| N-isopropyl-2- ((1r,3r)-3- (3-(2-(3- methylisoxazol- 5-yl) acetamido)- 1H-pyrazol-5- | 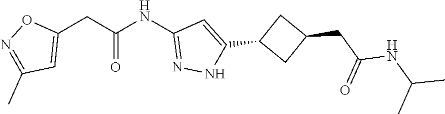 [SMILES: Cc1cc(CC(=O)Nc2cc([C@H]3C[C@H](CC(=O)NC(C)C)C3)[nH]n2)on1] | ¹H NMR (400 MHz, DMSO-d₆) 12.12 (s, 1H), 10.64 (s, 1H), 7.68 (d, J = 7.7 Hz, 1H), 6.34 (s, 1H), 6.23 (s, 1H), 3.90-3.71 (m, 3H), 3.47 (p, J = 7.7 Hz, 1H), 2.71- 2.54 (m, 1H), 2.26 (d, J = 7.8 Hz, 2H), 2.21-2.11 (m, 5H), 2.06 | 360.16 |
| yl)cyclobutyl) | | (ddd, J = 11.5, 8.6, 5.2 Hz, 2H), | |
| acetamide | | 1.03 (d, J = 6.6 Hz, 6H). | |
| N-(5-((1s,3s)- 3-(2- (isopropyl- amino)-2- oxoethyl) cyclobutyl)-1H- pyrazol-3-yl)-3- (methoxymethy 1)-1-methyl-1H- pyrazole-5- carboxamide | 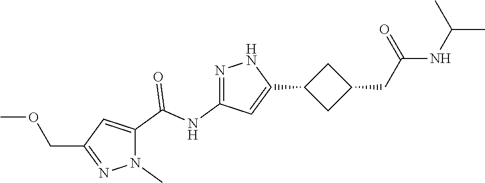 [SMILES: COCc1cc(C(=O)Nc2cc([C@H]3C[C@@H](CC(=O)NC(C)C)C3)[nH]n2)n(C)n1] | ¹H NMR (400 MHz, DMSO-d₆) 1.03 (d, J = 6.6 Hz, 6H), 1.83 (dt, J = 12.2, 9.6 Hz, 2H), 2.18 (d, J = 7.3 Hz, 2H), 2.40 (qd, J = 8.1, 2.7 Hz, 2H), 2.52-2.58 (m, 1H), 3.27 (s, 3H), 3.35 (s, 1H), 3.75-3.88 (m, 1H), 4.06 (s, 3H), 4.34 (s, 2H), 6.46 (s, 1H), 7.13 (s, 1H), 7.64 (d, J = 7.7 Hz, 1H), 10.73 (s, 1H), 12.19 (s, 1H). | 389.35 |
| N-(5-((1r,3r)-3-(2- (isopropylamino)- 2-oxoethyl) cyclobutyl)-1H- pyrazol-3-yl)-3- (methoxymethy 1)-1-methyl-1H- pyrazole-5- carboxamide | 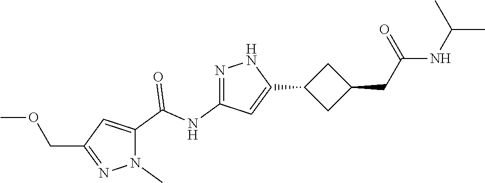 [SMILES: COCc1cc(C(=O)Nc2cc([C@H]3C[C@H](CC(=O)NC(C)C)C3)[nH]n2)n(C)n1] | ¹H NMR (400 MHz, CDCl₃) 1.12 (dd, J = 6.7, 2.8 Hz, 6H), 2.17 (ddt, J = 12.2, 8.7, 4.7 Hz, 2H), 2.35 (dd, J = 7.8, 2.4 Hz, 4H), 2.95 (dq, J = 23.1, 7.7, 6.7 Hz, 1H), 3.34 (d, J = 3.9 Hz, 3H), 3.49 (tt, J = 8.9, 5.2 Hz, 1H), 4.04 (td, J = 7.6, 3.7 Hz, 1H), 4.13 (d, J = 2.9 Hz, 3H), 4.39 (d, J = 3.6 Hz, 2H), 5.65-5.8 (m, 1H), 6.67 (d, J = 4.3 Hz, | 389.35 |
| | | 1H), 6.82 (d, J = 3.3 Hz, 1H), | |
| | | 10.23 (s, 1H), 11.73 (s, 1H). | |

### Example 2

### rel-(1R,4S)-4-(3-(2-(3-methylisoxazol-5-yl)acetamido)-1H-pyrazol-5-yl)cycloheptyl (1-methylcyclopropyl)carbamate

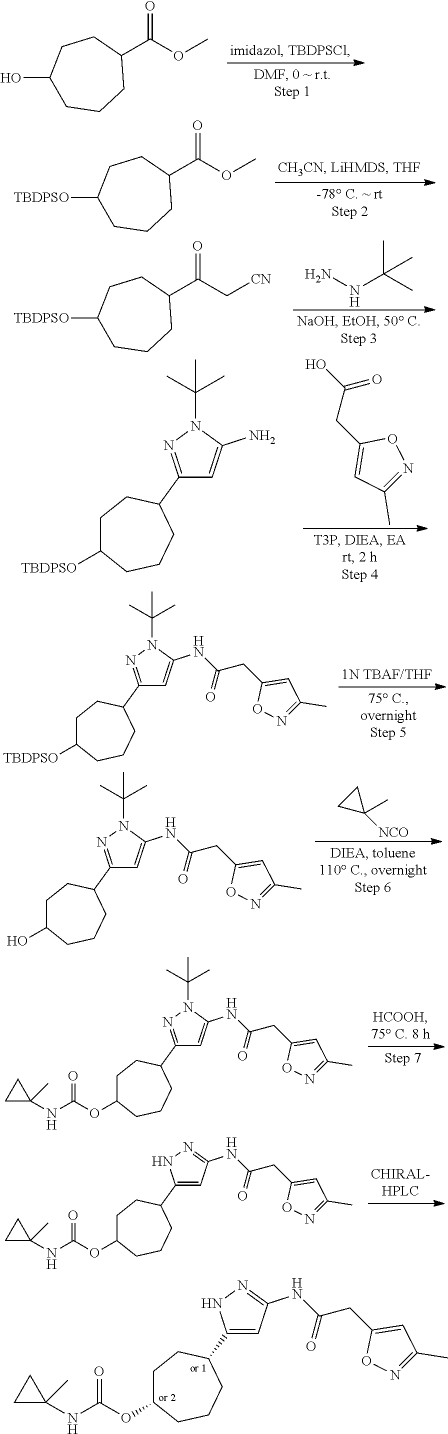

### methyl 4-((tert-butyldiphenylsilyl)oxy)cycloheptane-1-carboxylate

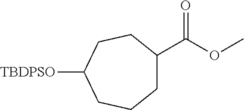

Step 1: To a mixture of methyl 4-hydroxycycloheptane-1-carboxylate (1 g, 6 mmol) and 1H-imidazole (1 g, 0.02 mol) in DMF (10 mL) was added tert-butylchlorodiphenylsilane (2 g, 7 mmol) dropwise at 0° C. under nitrogen atmosphere. The mixture was stirred for 12 hours at 25° C. The reaction mixture was diluted with water (20 mL), and the aqueous phase was extracted with EA (20 mL) three times. The combined organic layers were washed with brine, dried over sodium sulfate, filtered, and concentrated in vacuo. The crude product was purified by silica gel chromatography eluting with PE/EA (ratio: 10/1). Concentration in vacuo resulted in methyl 4-((tert-butyl diphenyl silyl)oxy)cycloheptane-1-carboxylate (2 g, 5 mmol, 80%) as a yellow oil. m/z (ES⁺) [M+H]⁺=411.20; HPLC tR=1.527 min.

### 3-(4-((tert-butyldiphenylsilyl)oxy)cycloheptyl)-3-oxopropanenitrile

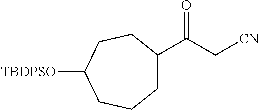

Step 2: To a solution of methyl 4-((tert-butyldiphenylsilyl)oxy)cycloheptane-1-carboxylate (1.8 g, 4.4 mmol) and acetonitrile (0.36 g, 8.8 mmol) in THE (20 mL) was added lithium bis(trimethylsilyl)amide (0.81 g, 4.8 mmol) dropwise at −78° C. under nitrogen atmosphere. The mixture was warmed to 25° C. and stirred for 1 h. The mixture was quenched with sat. NH₄Cl. The reaction mixture was diluted with water (20 mL), and the aqueous phase was extracted with EA (20 mL) three times. The combined organic layers were washed with brine, dried over sodium sulfate, filtered, and concentrated in vacuo. The crude product was purified by silica gel chromatography eluting with PE/EA (ratio:30/1). Concentration in vacuo resulted in 3-(4-((tert-butyldiphenylsilyl)oxy)cycloheptyl)-3-oxopropanenitrile (1.7 g, 4.1 mmol, 92%) as a yellow oil. m/z (ES⁺) [M+H]⁺=420.10; HPLC tR=1.280 min.

### 1-(tert-butyl)-3-(4-((tert-butyldiphenylsilyl)oxy)cycloheptyl)-1H-pyrazol-5-amine

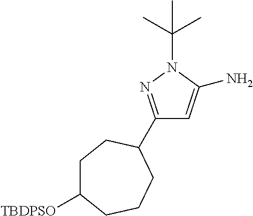

Step 3: A round bottomed flask was charged with tert-butylhydrazine hydrochloride (0.76 g, 6.1 mmol), sodium hydroxide (0.24 g, 6.1 mmol), EtOH (18 mL) and a stirbar. The solution was stirred for 1 hour at 25° C. 3-(4-((tert-butyldiphenylsilyl)oxy)cycloheptyl)-3-oxopropanenitrile (1.7 g, 4.1 mmol) was added, and the solution was stirred for 6 hours at 50° C. The mixture was quenched with water (20 mL), and the aqueous phase was extracted with EA (20 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 40% to 70% gradient in 12 min; detector, UV 254 nm to afford 1-(tert-butyl)-3-(4-((tert-butyldiphenylsilyl)oxy)cycloheptyl)-1H-pyrazol-5-amine (1.9 g, 3.9 mmol, 96%) as a yellow oil. m/z (ES⁺) [M+H]⁺=490.50; HPLC tR=1.305 min.

### N-(1-(tert-butyl)-3-(4-((tert-butyldiphenylsilyl)oxy)cycloheptyl)-1H-pyrazol-5-yl)-2-(3-methylisoxazol-5-yl)acetamide

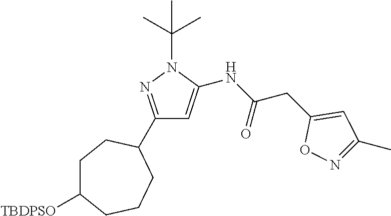

Step 4: A round bottomed flask was charged with 1-(tert-butyl)-3-(4-((tert-butyldiphenylsilyl)oxy)cycloheptyl)-1H-pyrazol-5-amine (1 g, 2 mmol), 2-(3-methylisoxazol-5-yl)acetic acid (0.3 g, 2 mmol), N-ethyl-N-isopropylpropan-2-amine (0.8 g, 6 mmol), EA (12 mL) and a stirbar. T3P in EA (2 g, 50% Wt, 3 mmol) was added, and the solution was stirred for 3 hours at 25° C. The reaction mixture was diluted with water (20 mL), and the aqueous phase was extracted with EA (20 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 80% to 100% gradient in 10 min; detector, UV 254 nm, to afford N-(1-(tert-butyl)-3-(4-((tert-butyldiphenylsilyl)oxy)cycloheptyl)-1H-pyrazol-5-yl)-2-(3-methylisoxazol-5-yl)acetamide (1.1 g, 1.8 mmol, 90%) as a yellow oil. m/z (ES⁺) [M+H]=613.25; HPLC tR=1.543 min

### N-(1-(tert-butyl)-3-(4-hydroxycycloheptyl)-1H-pyrazol-5-yl)-2-(3-methylisoxazol-5-yl)acetamide

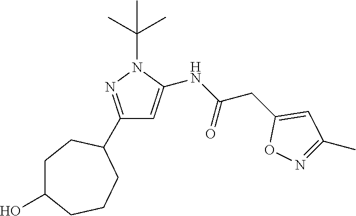 [SMILES: Cc1cc(CC(=O)Nc2cc(C3CCCC(O)CC3)nn2C(C)(C)C)on1]

Step 5: A round bottomed flask was charged with N-(1-(tert-butyl)-3-(4-((tert-butyldiphenylsilyl)oxy)cycloheptyl)-1H-pyrazol-5-yl)-2-(3-methylisoxazol-5-yl)acetamide (1.1 g, 1.8 mmol) and a stirbar. 1N TBAF in THF (12 mL) was added, and the solution was stirred for 16 hour at 75° C. The mixture was quenched with water (20 mL), and was extracted with EA (20 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 20% to 70% gradient in 15 min; detector, UV 254 nm to afford N-(1-(tert-butyl)-3-(4-hydroxycycloheptyl)-1H-pyrazol-5-yl)-2-(3-methylisoxazol-5-yl)acetamide (450 mg, 1.20 mmol, 67%) as a yellow oil. m/z (ES⁺) [M+H]⁺=375.15; HPLC tR=0.860 min.

### 4-(1-(tert-butyl)-5-(2-(3-methylisoxazol-5-yl)acetamido)-1H-pyrazol-3-yl)cycloheptyl (1-methylcyclopropyl)carbamate

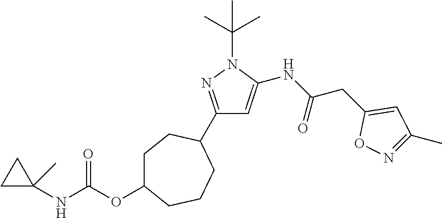 [SMILES: Cc1cc(CC(=O)Nc2cc(C3CCCC(OC(=O)NC4(C)CC4)CC3)nn2C(C)(C)C)on1]

Step 6: A round bottomed flask was charged with N-(1-(tert-butyl)-3-(4-hydroxycycloheptyl)-1H-pyrazol-5-yl)-2-(3-methylisoxazol-5-yl)acetamide (790 mg, 2.11 mmol), 1-isocyanato-1-methylcyclopropane (6.8 mL, 0.62 M in toluene, 4.22 mmol), N-ethyl-N-isopropylpropan-2-amine (818 mg, 6.33 mmol), toluene (10 mL) and a stirbar. The solution was stirred for 20 hours at 110° C. The mixture was concentrated in vacuo. The residue was purified by reverse flash chromatography with the following conditions: column, C18 silica gel; mobile phase, MeCN in water, 20% to 60% gradient in 10 min; detector, UV 220 nm to afford 4-(1-(tert-butyl)-5-(2-(3-methylisoxazol-5-yl)acetamido)-1H-pyrazol-3-yl)cycloheptyl (1-methylcyclopropyl)carbamate (800 mg, 1.70 mmol, 80.4%) as a yellow oil. m/z (ES⁺) [M+H]⁺=472.15; HPLC tR=1.098 min 4-(3-(2-(3-methylisoxazol-5-yl)acetamido)-1H-pyrazol-5-yl)cycloheptyl (1-methylcyclopropyl)carbamate

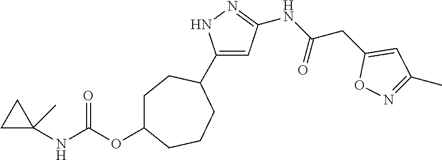 [SMILES: Cc1cc(CC(=O)Nc2cc(C3CCCC(OC(=O)NC4(C)CC4)CC3)[nH]n2)on1]

Step 7: A round bottomed flask was charged with 4-(1-(tert-butyl)-5-(2-(3-methylisoxazol-5-yl)acetamido)-1H-pyrazol-3-yl)cycloheptyl (1-methylcyclopropyl)carbamate (870 mg, 1.84 mmol) and a stirbar. HCOOH (8 mL) was added, and the solution was stirred for 8 hours at 75° C. The mixture was concentrated in vacuo. The residue was purified by reverse flash chromatography with the following conditions: column, C18 silica gel; mobile phase, MeCN in water, 20% to 70% gradient in 15 min; detector, UV 220 nm to afford 4-(3-(2-(3-methylisoxazol-5-yl)acetamido)-1H-pyrazol-5-yl)cycloheptyl (1-methylcyclopropyl)carbamate (650 mg, 1.56 mmol, 84.8%) as a yellow amorphous solid. m/z (ES⁺) [M+H]⁺=416.40; HPLC tR=0.926 min

### rel-(1R,4S)-4-(3-(2-(3-methylisoxazol-5-yl)acetamido)-1H-pyrazol-5-yl)cycloheptyl (1-methylcyclopropyl)carbamate

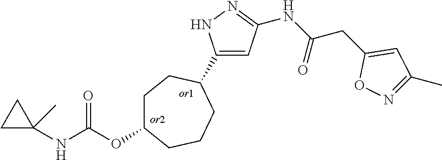 [SMILES: Cc1cc(CC(=O)Nc2cc([C@@H]3CCC[C@H](OC(=O)NC4(C)CC4)CC3)[nH]n2)on1]

Step 8: The 4-(3-(2-(3-methylisoxazol-5-yl)acetamido)-1H-pyrazol-5-yl)cycloheptyl (1-methylcyclopropyl) carbamate (650 mg, 1.56 mmol) was purified by chiral Pre-HPLC (Column: CHIRALPAK IF, 2*25 cm, 5 m; Mobile Phase A: Hex(0.2% TEA)—HPLC, Mobile Phase B: MeOH: DCM=1:1; Flow rate: 20 mL/min; Gradient: 60% B to 60% B in 21 min; Wave Length: 220/254 nm; RT1(min): 7.33; RT2(min): 13.51; Sample Solvent: MeOH: DCM=1:1; Injection Volume: 0.3 mL; Number Of Runs: 9). Lyophilization yielded rel-(1R,4S)-4-(3-(2-(3-methylisoxazol-5-yl)acetamido)-1H-pyrazol-5-yl)cycloheptyl (1-methylcyclopropyl)carbamate (31.1 mg, 74.9 μmol, 4.78%) as a white amorphous solid. m/z (ES⁺) [M+H]⁺=416.35; HPLC tR=0.909 min. ¹H NMR (400 MHz, DMSO-d₆) δ 12.06 (s, 1H), 10.62 (s, 1H), 7.32 (s, 1H), 6.27 (s, 1H), 6.22 (s, 1H), 4.74 (s, 1H), 3.83 (s, 2H), 2.97-2.66 (m, 1H), 2.21 (s, 3H), 1.94 (d, J=7.4 Hz, 2H), 1.77 (s, 5H), 1.58 (d, J=11.2 Hz, 2H), 1.39 (s, 1H), 1.24 (s, 3H), 0.59 (s, 2H), 0.47 (s, 2H).

Additional compounds prepared according to the methods of Example 2 are depicted in Table 3 below.

TABLE 3
Additional Exemplary Compounds

| | | | MS |
|---|---|---|---|
| Compound | Structure | Proton NMR | [M + 1] |
| rel-(1R,4S)-4- (3-(2-(3- methylisoxazol- 5- yl)acetamido)- 1H-pyrazol-5- yl)cycloheptyl (1- methylcyclo- propyl) carbamate | 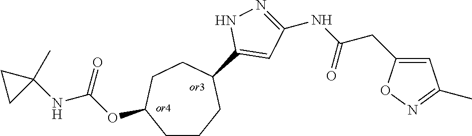 [SMILES: Cc1cc(CC(=O)Nc2cc([C@H]3CCC[C@@H](OC(=O)NC4(C)CC4)CC3)[nH]n2)on1] | ¹H NMR (400 MHz, DMSO-d₆) δ 12.06 (s, 1H), 10.62 (s, 1H), 7.19 (d, J = 96.8 Hz, 1H), 6.25 (d, J = 18.0 Hz, 2H), 4.74 (s, 1H), 3.83 (s, 2H), 2.81 (s, 1H), 2.21 (s, 3H), 1.94 (d, J = 8.6 Hz, 2H), 1.77 (s, 5H), 1.62-1.44 (m, 2H), 1.40 (s, 1H), 1.24 (s, 3H), 0.59 (s, 2H), 0.47 (s, 2H). | 416.35 |
| rel-(1R,4R)-4- (3-(2-(3- methylisoxazol- 5- yl)acetamido)- 1H-pyrazol-5- yl)cycloheptyl (1- methylcyclo- propyl) carbamate | 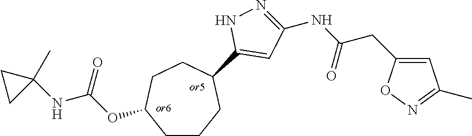 [SMILES: Cc1cc(CC(=O)Nc2cc([C@H]3CCC[C@H](OC(=O)NC4(C)CC4)CC3)[nH]n2)on1] | ¹H NMR (400 MHz, DMSO-d₆) δ 12.06 (s, 1H), 10.61 (s, 1H), 7.31 (s, 1H), 6.24 (d, J = 11.4 Hz, 2H), 4.73 (d, J = 40.3 Hz, 1H), 3.83 (s, 2H), 2.82 (s, 1H), 2.20 (s, 3H), 1.89 (t, J = 18.2 Hz, 4H), 1.57 (s, 6H), 1.24 (s, 3H), 0.59 (d, J = 5.2 Hz, 2H), 0.48 (d, J = 5.1 Hz, 2H). | 416.35 |
| rel-(1R,4R)-4- (3-(2-(3- methylisoxazol- 5- yl)acetamido)- 1H-pyrazol-5- yl)cycloheptyl (1- methylcyclo- propyl) carbamate | 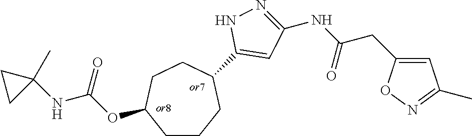 [SMILES: Cc1cc(CC(=O)Nc2cc([C@@H]3CCC[C@@H](OC(=O)NC4(C)CC4)CC3)[nH]n2)on1] | ¹H NMR (400 MHz, DMSO-d₆) δ 2.05 (s, 1H), 10.62 (s, 1H), 7.31 (s, 1H), 6.24 (d, J = 13.3 Hz, 2H), 4.69 (s, 1H), 3.83 (s, 2H), 2.82 (s, 1H), 2.20 (s, 3H), 2.02-1.76 (m, 4H), 1.58 (s, 6H), 1.24 (s, 3H), 0.64-0.55 (m, 2H), 0.48 (d, J = 5.0 Hz, 2H). | 416.4 |
| N-(3-((1R,3R)- 3- hydroxycyclo- pentyl)-1H- pyrazol-5-yl)-2- (3- methylisoxazol- 5-yl)acetamide | 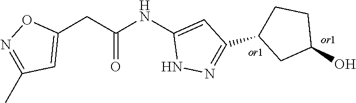 [SMILES: Cc1cc(CC(=O)Nc2cc([C@@H]3CC[C@@H](O)C3)n[nH]2)on1] | ¹H NMR (400 MHz, DMSO-d₆) 1.42-1.59 (m, 2H), 1.65 (ddd, J = 12.9, 10.3, 5.5 Hz, 1H), 1.85- 2.01 (m, 2H), 2.08 (dt, J = 11.9, 7.3 Hz, 1H), 2.20 (s, 3H), 3.19- 3.3 (m, 1H), 3.82 (s, 2H), 4.23 (dq, J = 5.9, 2.9 Hz, 1H), 4.56 (d, J = 3.5 Hz, 1H), 6.23 (d, J = 9.3 Hz, 2H), 10.63 (s, 1H), 12.10 (s, 1H). | 291.10 |
| N-(3-((1S,3S)- 3- hydroxycyclo- pentyl)-1H- pyrazol-5-yl)-2- (3- methylisoxazol- 5-yl)acetamide | 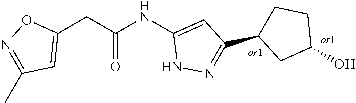 [SMILES: Cc1cc(CC(=O)Nc2cc([C@H]3CC[C@H](O)C3)n[nH]2)on1] | ¹H NMR (400 MHz, DMSO-d₆) 1.42-1.57 (m, 2H), 1.65 (ddd, J = 12.8, 10.3, 5.5 Hz, 1H), 1.85- 2.01 (m, 2H), 2.02-2.16 (m, 1H), 2.20 (s, 3H), 3.21-3.3 (m, 1H), 3.82 (s, 2H), 4.2-4.27 (m, 1H), 4.56 (d, J = 3.5 Hz, 1H), 6.23 (d, J = 10.2 Hz, 2H), 10.62 (s, 1H), 12.10 (s, 1H). | 291.00 |

### Example 3

### (1R,3S)-3-methyl-3-(3-(2-(3-methylisoxazol-5-yl)acetamido)-1H-pyrazol-5-yl)cyclopentyl (1-methylcyclopropyl)carbamate

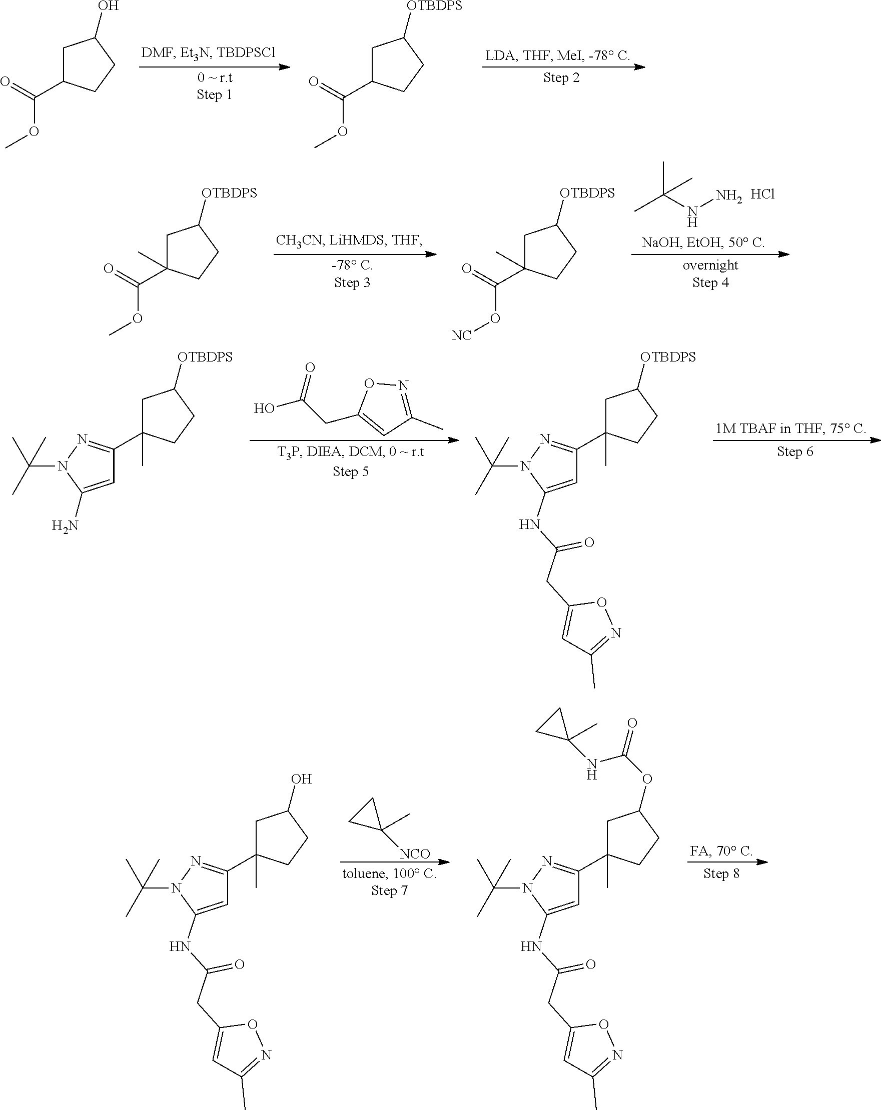

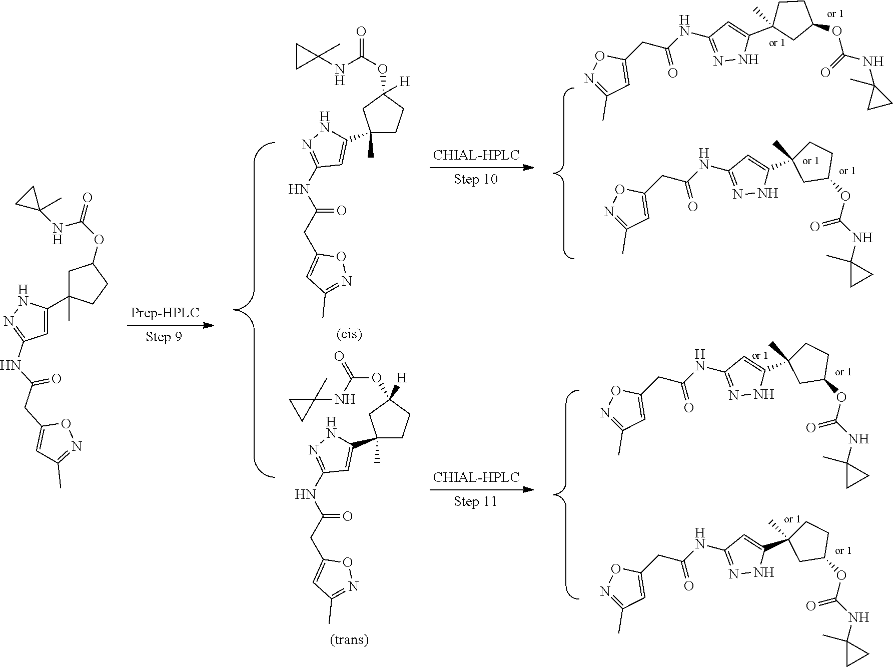

### methyl 3-((tert-butyldiphenylsilyl)oxy)cyclopentane-1-carboxylate

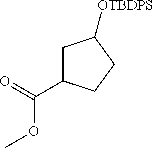

Step 1: To an ice cooled solution of methyl 3-hydroxycyclopentane-1-carboxylate (3.5 g, 24 mmol) and Et₃N (7.4 g, 10 mL, 73 mmol) in DMF (40 mL), TBDPS-Cl (10 g, 36 mmol) was added, and then the resulting mixture was stirred at room temperature overnight. The resulting mixture was diluted with EA(100 mL), and washed with brine (3*80 mL). The organic layers were dried over Na₂SO₄ and concentrated under vacuum. The residue was purified by silica gel chromatography (eluting with 0-15% EA in petroleum ether) to afford methyl 3-((tert-butyldiphenylsilyl)oxy)cyclopentane-1-carboxylate (8.8 g, 23 mmol, 95%) as a light solid. m/z (ES⁺) [M+H]⁺=383.15; HPLC tR=1.425 min.

### methyl 3-((tert-butyldiphenylsilyl)oxy)-1-methylcyclopentane-1-carboxylate

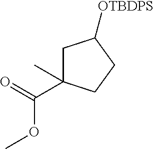

Step 2: To a solution of methyl 3-((tert-butyldiphenylsilyl)oxy)cyclopentane-1-carboxylate (8.8 g, 23 mmol) in THE (100 mL) at −78° C., a solution of LDA (13.7 mL, 2 M in THF, 27.6 mmol) was added. The resulting mixture was stirred at −78° C. for 0.5 h, and then Mel (16 g, 0.12 mol) was added to the above mixture at −78° C. After stirring for an additional 1 hour, the reaction mixture was allowed to warm to room temperature. The resulting mixture was quenched with NH₄Cl (sat.aq. 150 mL), and extracted with EA (3*150 mL). The combined organic layers were washed with brine (2*100 mL), dried over Na₂SO₄ and concentrated under vacuum. The residue was purified by silica gel chromatography (eluting with 0˜15% EA in petroleum ether) to afford methyl 3-((tert-butyldiphenylsilyl)oxy)-1-methylcyclopentane-1-carboxylate (8.5 g, 21 mmol, 93%) as a light oil. m/z (ES⁺) [M+H]⁺=397.20; HPLC tR=1.507 min.

### 3-(3-((tert-butyldiphenylsilyl)oxy)-1-methylcyclopentyl)-3-oxopropanenitrile

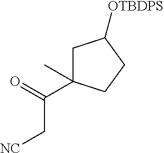

Step 3: To a solution of methyl 3-((tert-butyldiphenylsilyl)oxy)-1-methylcyclopentane-1-carboxylate (8.5 g 21 mmol) and MeCN (1.1 g, 26 mmol) in THE (80 mL), LiHMDS (21 mL, 1 M in THF, 21 mmol) was added dropwise, slowly enough to maintain the internal temperature below −60° C. After stirring for 1 hour at −70° C., the reaction was quenched with sat. NH₄Cl (100 mL), and extracted with EA(3*100 mL). The combined organic layers were washed with brine (2*100 mL), dried over Na₂SO₄, filtered, and concentrated under vacuum to afford 3-(3-((tert-butyldiphenylsilyl)oxy)-1-methylcyclopentyl)-3-oxopropanenitrile (8 g, 0.02 mol, 90%) as a light oil. m/z (ES⁺) [M+H]⁺=406; HPLC tR=1.507 min.

### 1-(tert-butyl)-3-(3-((tert-butyldiphenylsilyl)oxy)-1-methylcyclopentyl)-1H-pyrazol-5-amine

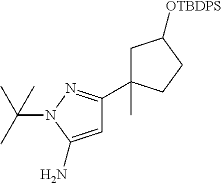

Step 4: Sodium hydroxide (0.2 g, 5 mmol) was added in portions to a suspension of tert-butylhydrazine hydrochloride (0.8 g, 6 mmol) in EtOH (0.5 mL) at room temperature, and stirred at room temperature for 1 hour. A solution of 3-(3-((tert-butyldiphenylsilyl)oxy)-1-methylcyclopentyl)-3-oxopropanenitrile (2 g, 5 mmol) in ethanol was added at room temperature, then the mixture was heated to 50° C. and stirred overnight. The reaction mixture was allowed to cool down to room temperature, filtered, and concentrated under vacuum. The residue was purified by reverse flash chromatography with the following conditions: column, C18 silica gel; mobile phase, MeCN in water, 0% to 100% gradient in 15 min; detector, UV 220 nm to give 1-(tert-butyl)-3-(3-((tert-butyldiphenylsilyl)oxy)-1-methylcyclopentyl)-1H-pyrazol-5-amine (0.95 g, 2.0 mmol, 40%) as a white solid. m/z (ES⁺) [M+H]⁺=476.40; HPLC tR=1.287 min.

### N-(1-(tert-butyl)-3-(3-((tert-butyldiphenylsilyl)oxy)-1-methylcyclopentyl)-1H-pyrazol-5-yl)-2-(3-methylisoxazol-5-yl)acetamide

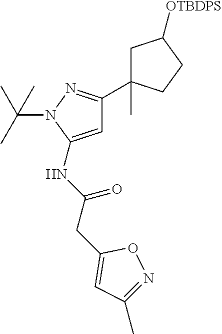

Step 5: To cooled mixture of 1-(tert-butyl)-3-(3-((tert-butyldiphenylsilyl)oxy)-1-methylcyclopentyl)-1H-pyrazol-5-amine (0.95 g, 2.0 mmol), 2-(3-methylisoxazol-5-yl)acetic acid (0.34 g, 2.4 mmol) and DIEA (0.77 g, 6.0 mmol) in DCM (10 mL), 2,4,6-tripropyl-1,3,5,2,4,6-trioxatriphosphinane 2,4,6-trioxide (3.8 g, 50% Wt solution in ethyl acetate, 6.0 mmol) was added. The resulting mixture was stirred for 2 hours at room temperature. The reaction was quenched with sat. aq Na₂CO₃(10 mL) and extracted with DCM (2*10 mL). the organic layer was washed with Na₂CO₃(2*10 mL), brine (30 mL), and concentrated. The residue was purified by reverse flash chromatography with the following conditions: column, C18 silica gel; mobile phase, MeCN in water, 0% to 100% gradient in 15 min; detector, UV 220 nm. This resulted in N-(1-(tert-butyl)-3-(3-((tert-butyldiphenylsilyl)oxy)-1-methylcyclopentyl)-1H-pyrazol-5-yl)-2-(3-methylisoxazol-5-yl)acetamide (1.05 g, 1.75 mmol, 88%) as a white solid. m/z (ES⁺) [M+H]⁺=599.50; HPLC tR=1.608 min.

### N-(1-(tert-butyl)-3-(3-hydroxy-1-methylcyclopentyl)-1H-pyrazol-5-yl)-2-(3-methylisoxazol-5-yl)acetamide

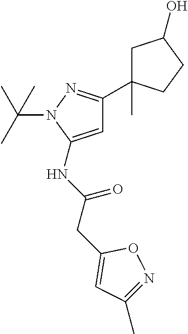 [SMILES: Cc1cc(CC(=O)Nc2cc(C3(C)CCC(O)C3)nn2C(C)(C)C)on1]

Step 6: A mixture of N-(1-(tert-butyl)-3-(3-((tert-butyldiphenylsilyl)oxy)-1-methylcyclopentyl)-1H-pyrazol-5-yl)-2-(3-methylisoxazol-5-yl)acetamide (1.05 g, 1.75 mmol) and TBAF (15 mL, 1 M in THF, 15 mmol) was stirred for 6 hours at 75° C. The reaction mixture was allowed to cool to room temperature, diluted with water (10 mL), and extracted with EA (3*150 mL). The combined organic layers were washed with brine (2*100 mL), dried over Na₂SO₄ and concentrated under vacuum. The residue was purified by reverse flash chromatography with the following conditions: column, C18 silica gel; mobile phase, MeCN in water, 0% to 100% gradient in 40 min; detector, UV 220 nm to give N-(1-(tert-butyl)-3-(3-hydroxy-1-methylcyclopentyl)-1H-pyrazol-5-yl)-2-(3-methylisoxazol-5-yl)acetamide (450 mg, 1.25 mmol, 71.2%) as white solid. m/z (ES⁺) [M+H]⁺=361.25; HPLC tR=0.817 min. 3-(1-(tert-butyl)-5-(2-(3-methylisoxazol-5-yl)acetamido)-1H-pyrazol-3-yl)-3-methylcyclopentyl (1-methylcyclopropyl)carbamate

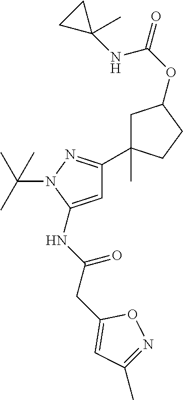 [SMILES: Cc1cc(CC(=O)Nc2cc(C3(C)CCC(OC(=O)NC4(C)CC4)C3)nn2C(C)(C)C)on1]

Step 7: To a stirred mixture of N-(1-(tert-butyl)-3-(3-hydroxy-1-methylcyclopentyl)-1H-pyrazol-5-yl)-2-(3-methylisoxazol-5-yl)acetamide (450 mg, 1.25 mmol) and 1-isocyanato-1-methylcyclopropane(0.6 M in toluene) (6 mL, 4 mmol) was added DIEA (484 mg, 3.75 mmol) dropwise at room temperature under nitrogen atmosphere. The resulting mixture was stirred for 16 hours at 100° C. under nitrogen atmosphere. Then the reaction mixture was allowed to cool down to room temperature and concentrated under reduced pressure. The residue was purified by reverse phase flash to afford 3-(1-(tert-butyl)-5-(2-(3-methylisoxazol-5-yl)acetamido)-1H-pyrazol-3-yl)-3-methylcyclopentyl (1-methylcyclopropyl)carbamate (480 mg, 1.05 mmol, 84.0%) as a light yellow oil. m/z (ES⁺) [M+H]⁺=458.40; HPLC tR=1.170 min

### 3-methyl-3-(3-(2-(3-methylisoxazol-5-yl)acetamido)-1H-pyrazol-5-yl)cyclopentyl (1-methylcyclopropyl) carbamate

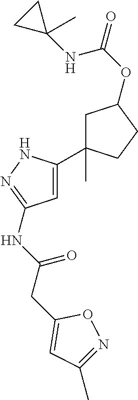 [SMILES: Cc1cc(CC(=O)Nc2cc(C3(C)CCC(OC(=O)NC4(C)CC4)C3)[nH]n2)on1]

Step 8: A solution of 3-(1-(tert-butyl)-5-(2-(3-methylisoxazol-5-yl)acetamido)-1H-pyrazol-3-yl)-3-methylcyclopentyl (1-methylcyclopropyl)carbamate (480 mg, 1.05 mmol) in FA (3 mL) was stirred at 70° C. for 4 hour. The resulting mixture was concentrated to dryness, and purified by reverse phase flash to afford 3-methyl-3-(3-(2-(3-methylisoxazol-5-yl)acetamido)-1H-pyrazol-5-yl)cyclopentyl (1-methylcyclopropyl) carbamate (360 mg, 897 μmol, 85.5%) as a white solid. m/z (ES⁺) [M+H]=402.20; HPLC tR=0.880 min.

### cis-3-methyl-3-(3-(2-(3-methylisoxazol-5-yl)acetamido)-1H-pyrazol-5-yl)cyclopentyl (1-methylcyclopropyl)carbamate and trans-3-methyl-3-(3-(2-(3-methylisoxazol-5-yl)acetamido)-1H-pyrazol-5-yl)cyclopentyl (1-methylcyclopropyl)carbamate

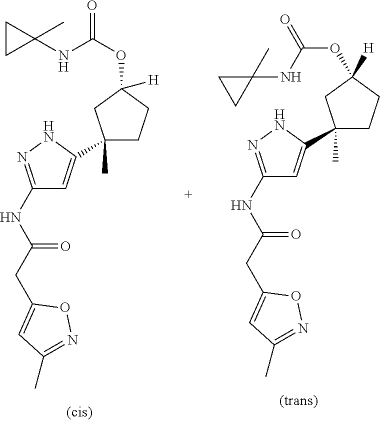 [SMILES: Cc1cc(CC(=O)Nc2cc([C@@]3(C)CC[C@@H](OC(=O)NC4(C)CC4)C3)[nH]n2)on1 ; Cc1cc(CC(=O)Nc2cc([C@]3(C)CC[C@@H](OC(=O)NC4(C)CC4)C3)[nH]n2)on1]

Step 9: 3-methyl-3-(3-(2-(3-methylisoxazol-5-yl)acetamido)-1H-pyrazol-5-yl)cyclopentyl (1-methylcyclopropyl) carbamate (360 mg, 897 μmol) was separated by Prep-HPLC with the following condition: Column: XBridge Prep OBD C18 Column, 30*150 mm, 5 m; Mobile Phase A: Water(10 mmol/L NH₄HCO₃+0.1% NH₃·H₂O), Mobile Phase B: ACN; Flow rate: 60 mL/min; Gradient: 20% B to 42% B in 8 min, 42% B; Wave Length: 220 nm; RT1(min): 6.97/7.93; Number Of Runs: 3 to afford cis-3-methyl-3-(3-(2-(3-methylisoxazol-5-yl)acetamido)-1H-pyrazol-5-yl)cyclopentyl (1-methylcyclopropyl)carbamate (60 mg, 0.15 mmol, 17%) as white solid and trans-3-methyl-3-(3-(2-(3-methylisoxazol-5-yl)acetamido)-1H-pyrazol-5-yl)cyclopentyl (1-methylcyclopropyl)carbamate (220 mg, 548 μmol, 61.1%) as white solid.

Cis: m/z (ES⁺) [M+H]⁺=402.35; HPLC tR=0.854 min.

Trans: m/z (ES⁺) [M+H]⁺=402.40; HPLC tR=0.894 min.

### (1R,3S)-3-methyl-3-(3-(2-(3-methylisoxazol-5-yl)acetamido)-1H-pyrazol-5-yl)cyclopentyl (1-methylcyclopropyl)carbamate

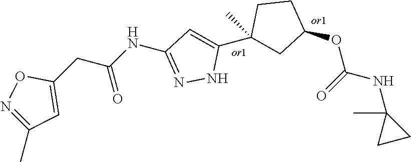 [SMILES: Cc1cc(CC(=O)Nc2cc([C@@]3(C)CC[C@@H](OC(=O)NC4(C)CC4)C3)[nH]n2)on1]

Step 10: Cis-3-methyl-3-(3-(2-(3-methylisoxazol-5-yl)acetamido)-1H-pyrazol-5-yl)cyclopentyl (1-methyl cyclopropyl)carbamate (100 mg, 249 μmol) was purified by Prep-CHIRAL-HPLC with the following condition: Column: CHIRALPAK IG, 2*25 cm, 5 m; Mobile Phase A: Hex(0.2% TEA)—HPLC, Mobile Phase B: EtOH: DCM=1:1—HPLC; Flow rate: 20 mL/min; Gradient: 70% B to 70% B in 15 min; Wave Length: 220/254 nm; RT1(min): 5.33; Sample Solvent: EtOH: DCM=1:1—HPLC; Injection Volume: 0.9 mL; Number Of Runs: 3. This afforded (1R,3S)-3-methyl-3-(3-(2-(3-methylisoxazol-5-yl)acetamido)-1H-pyrazol-5-yl)cyclopentyl (1-methylcyclopropyl)carbamate (34 mg, 85 μmol, 34%) as a white amorphous solid. m/z (ES⁺) [M+H]⁺=402.35; HPLC tR=0.861 min. ¹H NMR (400 MHz, DMSO-d₆) δ 12.08 (s, 1H), 10.61 (s, 1H), 7.16 (d, J=103.7 Hz, 1H), 6.26 (d, J=28.0 Hz, 2H), 5.05 (s, 1H), 3.83 (s, 2H), 2.20 (s, 3H), 2.18-2.04 (m, 2H), 1.99 (s, 1H), 1.85 (d, J=14.0 Hz, 1H), 1.69 (s, 2H), 1.23 (d, J=8.9 Hz, 6H), 0.58 (s, 2H), 0.45 (s, 2H).

### (1R,3R)-3-methyl-3-(3-(2-(3-methylisoxazol-5-yl)acetamido)-1H-pyrazol-5-yl)cyclopentyl (1-methylcyclopropyl)carbamate

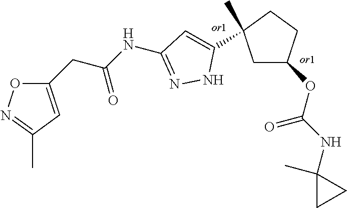 [SMILES: Cc1cc(CC(=O)Nc2cc([C@]3(C)CC[C@@H](OC(=O)NC4(C)CC4)C3)[nH]n2)on1]

Trans-3-methyl-3-(3-(2-(3-methylisoxazol-5-yl)acetamido)-1H-pyrazol-5-yl)cyclopentyl (1-methylcyclopropyl)carbamate (220 mg, 548 μmol) was purified by Prep-CHIRAL-HPLC with the following condition: Column: CHIRALPAK IH, 2*25 cm, 5 m; Mobile Phase A: Hex(0.2% TEA)—HPLC, Mobile Phase B: EtOH: DCM=1:1—HPLC; Flow rate: 20 mL/min; Gradient: 30% B to 30% B in 8 min; Wave Length: 220/254 nm; RT1(min): 5.03; Sample Solvent: EtOH: DCM=1:1—HPLC; Injection Volume: 0.4 mL; Number Of Runs: 6. This afforded (1R,3R)-3-methyl-3-(3-(2-(3-methylisoxazol-5-yl)acetamido)-1H-pyrazol-5-yl)cyclopentyl (1-methylcyclopropyl)carbamate (81.3 mg, 203 μmol, 37.0%) as white amorphous solid.

m/z (ES⁺) [M+H]⁺=402.35; HPLC tR=0.900 min.

¹H NMR (400 MHz, DMSO-d₆) δ 12.12 (s, 1H), 10.63 (s, 1H), 7.23 (d, J=88.4 Hz, 1H), 6.26 (d, J=27.4 Hz, 2H), 5.02 (d, J=41.5 Hz, 1H), 3.83 (s, 2H), 2.39-2.25 (m, 1H), 2.21 (s, 3H), 2.10-1.97 (m, 1H), 1.91 (d, J=6.5 Hz, 1H), 1.72 (d, J=44.1 Hz, 3H), 1.35 (s, 3H), 1.24 (s, 3H), 0.61 (d, J=6.1 Hz, 2H), 0.48 (d, J=5.2 Hz, 2H).

### (1S,3R)-3-methyl-3-(3-(2-(3-methylisoxazol-5-yl)acetamido)-1H-pyrazol-5-yl)cyclopentyl (1-methylcyclopropyl)carbamate

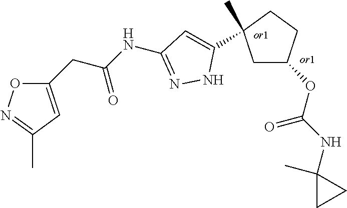 [SMILES: Cc1cc(CC(=O)Nc2cc([C@]3(C)CC[C@H](OC(=O)NC4(C)CC4)C3)[nH]n2)on1]

Cis-3-methyl-3-(3-(2-(3-methylisoxazol-5-yl)acetamido)-1H-pyrazol-5-yl)cyclopentyl (1-methylcyclopropyl)carbamate (100 mg, 249 μmol) was purified by Prep-CHIRAL-HPLC with the following condition: Column: CHIRALPAK IG, 2*25 cm, 5 m; Mobile Phase A: Hex(0.2% TEA)—HPLC, Mobile Phase B: EtOH: DCM=1:1—HPLC; Flow rate: 20 mL/min; Gradient: 70% B to 70% B in 15 min; Wave Length: 220/254 nm;RT2(min): 10.65; Sample Solvent: EtOH: DCM=1:1—HPLC; Injection Volume: 0.9 mL; Number Of Runs: 3. This afforded (1S,3R)-3-methyl-3-(3-(2-(3-methylisoxazol-5-yl)acetamido)-1H-pyrazol-5-yl)cyclopentyl (1-methylcyclopropyl)carbamate (34 mg, 85 μmol, 34%) as white amorphous solid.

m/z (ES⁺) [M+H]⁺=402.35; HPLC tR=0.865 min.

¹H NMR (400 MHz, DMSO-d₆) δ 12.08 (s, 1H), 10.62 (s, 1H), 7.29 (s, 1H), 6.26 (d, J=26.7 Hz, 2H), 5.05 (s, 1H), 3.83 (s, 2H), 2.20 (s, 3H), 2.16-2.03 (m, 2H), 1.98 (s, 1H), 1.86 (s, 1H), 1.73-1.59 (m, 2H), 1.23 (d, J=8.7 Hz, 6H), 0.51 (d, J=49.6 Hz, 4H).

### (1S,3S)-3-methyl-3-(3-(2-(3-methylisoxazol-5-yl)acetamido)-1H-pyrazol-5-yl)cyclopentyl (1-methylcyclopropyl)carbamate

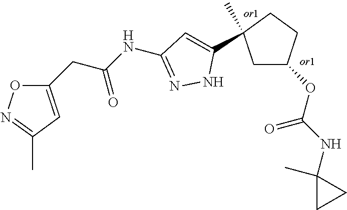 [SMILES: Cc1cc(CC(=O)Nc2cc([C@@]3(C)CC[C@H](OC(=O)NC4(C)CC4)C3)[nH]n2)on1]

Trans-3-methyl-3-(3-(2-(3-methylisoxazol-5-yl)acetamido)-1H-pyrazol-5-yl)cyclopentyl (1-methylcyclopropyl)carbamate (220 mg, 548 μmol) was purified by Prep-CHIRAL-HPLC with the following condition: Column: CHIRALPAK IH, 2*25 cm, 5 m; Mobile Phase A: Hex(0.2% TEA)—HPLC, Mobile Phase B: EtOH: DCM=1:1—HPLC; Flow rate: 20 mL/min; Gradient: 30% B to 30% B in 8 min; Wave Length: 220/254 nm; RT2(min): 7.26; Sample Solvent: EtOH: DCM=1:1—HPLC; Injection Volume: 0.4 mL; Number Of Runs: 6. This afforded (1S,3S)-3-methyl-3-(3-(2-(3-methylisoxazol-5-yl)acetamido)-1H-pyrazol-5-yl)cyclopentyl (1-methylcyclopropyl)carbamate (86.8 mg, 216 μmol, 39.5%) as white amorphous solid.

m/z (ES⁺) [M+H]⁺=402.35; HPLC tR=0.898 min.

¹H NMR (400 MHz, DMSO-d₆) δ 12.12 (s, 1H), 10.63 (s, 1H), 7.34 (s, 1H), 6.26 (d, J=27.2 Hz, 2H), 5.02 (d, J=42.9 Hz, 1H), 3.83 (s, 2H), 2.42-2.25 (m, 1H), 2.21 (s, 3H), 2.01 (s, 1H), 1.92 (t, J=9.4 Hz, 1H), 1.72 (d, J=46.0 Hz, 3H), 1.35 (s, 3H), 1.24 (s, 3H), 0.60 (s, 2H), 0.48 (s, 2H).

Additional compounds prepared according to the methods of Example 3 are depicted in Table 4 below.

TABLE 4
Additional Exemplary Compounds

| | | | MS |
|---|---|---|---|
| Compound | Structure | Proton NMR | [M + 1] |
| (1s,3s)-3-(3-(2-(3- methylisoxazol-5- yl)acetamido)-1H- pyrazol-5-yl)cyclobutyl isopropylcarbamate | 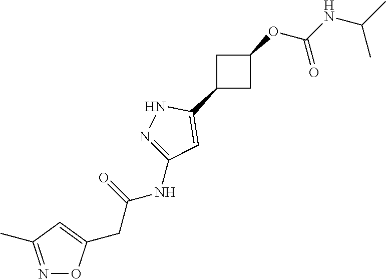 [SMILES: Cc1cc(CC(=O)Nc2cc([C@H]3C[C@@H](OC(=O)NC(C)C)C3)[nH]n2)on1] | ¹H NMR (400 MHz, DMSO-d₆) 12.19 (s, 1H), 10.64 (s, 1H), 7.06 (d, J = 7.9 Hz, 1H), 6.32 (s, 1H), 6.23 (s, 1H), 4.80 (p, J = 7.7 Hz, 1H), 3.83 (s, 2H), 3.62-3.52 (m, 1H), 3.04 (q, J = 8.9 Hz, 1H), 2.66 (d, J = 9.5 Hz, 2H), 2.21 (s, 3H), 2.05 (t, J = 10.0 Hz, 2H), 1.04 (d, J = 6.6 Hz, 6H). | 362.05 |
| ((1s,4s)-4-(3-(2-(3- methylisoxazol-5- yl)acetamido)-1H- pyrazol-5- yl)cyclohexyl)methyl isopropylcarbamate | 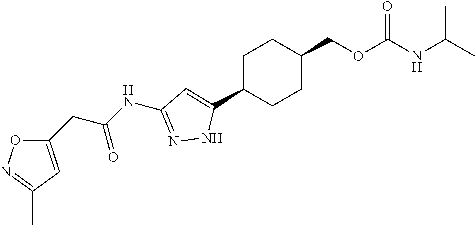 [SMILES: Cc1cc(CC(=O)Nc2cc([C@H]3CC[C@@H](COC(=O)NC(C)C)CC3)[nH]n2)on1] | ¹H NMR (400 MHz, DMSO- d₆) 12.07 (s, 1H), 10.64 (s, 1H), 6.95 (d, J = 8.1 Hz, 1H), 6.31 (s, 1H), 6.23 (s, 1H), 3.85 (d, J = 16.8 Hz, 4H), 3.57 (dd, J = 13.4, 6.8 Hz, 1H), 2.80 (s, 1H), 2.20 (s, 3H), 1.78 (s, 3H), 1.66 (td, J = 8.8, 4.2 Hz, 2H), 1.54 (ddd, J = 13.0, 8.2, 4.0 Hz, 2H), 1.41-1.36 (m, 2H), 1.03 (d, J = 6.6 Hz, 6H). | 404.20 |
| ((1r,3r)-3-(3-(2-(3- methylisoxazol-5- yl)acetamido)-1H- pyrazol-5- yl)cyclobutyl)methyl isopropylcarbamate | 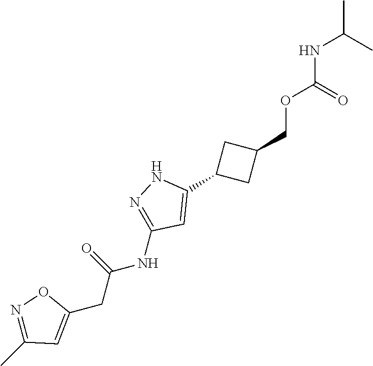 [SMILES: Cc1cc(CC(=O)Nc2cc([C@H]3C[C@H](COC(=O)NC(C)C)C3)[nH]n2)on1] | ¹H NMR (400 MHz, DMSO- d₆) 12.14 (s, 1H), 10.64 (s, 1H), 7.05 (d, J = 7.9 Hz, 1H), 6.35 (s, 1H), 6.23 (s, 1H), 4.04 (d, J = 7.2 Hz, 2H), 3.83 (s, 2H), 3.57 (ddt, J = 21.7, 15.8, 7.4 Hz, 3H), 2.21 (s, 4H), 2.16 (d, J = 8.1 Hz, 3H), 1.05 (d, J = 6.5 Hz, 6H). | 376 |
| ((1S,2R)-2-(3-(2-(3- methylisoxazol-5- yl)acetamido)-1H- pyrazol-5- yl)cyclopropyl)methyl isopropylcarbamate | 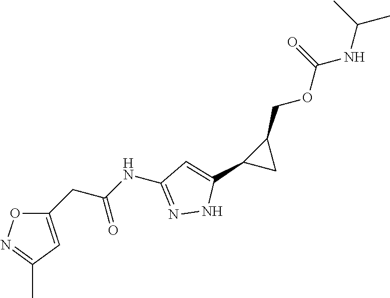 [SMILES: Cc1cc(CC(=O)Nc2cc([C@@H]3C[C@@H]3COC(=O)NC(C)C)[nH]n2)on1] | ¹H NMR (400 MHz, DMSO- d₆) 12.17 (s, 1H), 10.63 (s, 1H), 6.99 (d, J = 7.6 Hz, 1H), 6.24 (d, J = 13.4 Hz, 2H), 3.83 (s, 2H), 3.69 (d, J = 7.4 Hz, 2H), 3.54 (h, J = 6.7 Hz, 1H), 2.20 (s, 3H), 2.10 (q, J = 8.1 Hz, 1H), 1.49-1.38 (m, 1H), 1.18- 1.07 (m, 1H), 1.01 (dd, J = 6.8, 4.3 Hz, 6H), 0.84 (d, J = 5.5 Hz, 1H). | 362.10 |
| ((1R,2S)-2-(3-(2-(3- methylisoxazol-5- yl)acetamido)-1H- pyrazol-5- yl)cyclopropyl)methyl isopropylcarbamate | 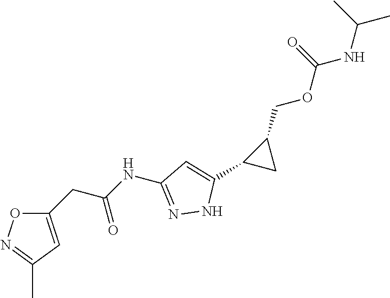 [SMILES: Cc1cc(CC(=O)Nc2cc([C@H]3C[C@H]3COC(=O)NC(C)C)[nH]n2)on1] | ¹H NMR (400 MHz, DMSO- d₆) 12.17 (s, 1H), 10.63 (s, 1H), 6.99 (d, J = 7.8 Hz, 1H), 6.24 (d, J = 12.3 Hz, 2H), 3.83 (s, 2H), 3.74-3.61 (m, 2H), 3.60- 3.47 (m, 1H), 2.20 (s, 3H), 2.10 (q, J = 8.1 Hz, 1H), 1.44 (dd, J = 15.0, 7.4 Hz, 1H), 1.13-1.06 (m, 1H), 1.01 (t, J = 5.5 Hz, 6H), 0.84 (d, J = 5.6 Hz, 1H). | 362.10 |
| (1s, 4s)-4-(3-(2-(3- methylisoxazol-5- yl)acetamido)-1H- pyrazol-5-yl)cyclohexyl isopropylcarbamate | 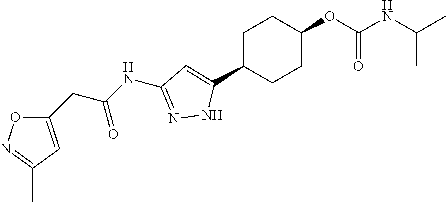 [SMILES: Cc1cc(CC(=O)Nc2cc([C@H]3CC[C@@H](OC(=O)NC(C)C)CC3)[nH]n2)on1] | ¹H NMR (400 MHz, DMSO- d₆) 12.11 (s, 1H), 10.65 (s, 1H), 6.94 (d, J = 7.6 Hz, 1H), 6.29 (s, 1H), 6.22 (s, 1H), 4.75 (s, 1H), 3.83 (s, 2H), 3.61- 3.55 (m, 1H), 2.67 (s, 1H), 2.20 (s, 3H), 1.78-1.71 (m, 5H), 1.64 (t, J = 14.2 Hz, 3H), 1.04 (d, J = 6.5 Hz, 6H). | 390.15 |
| ((1s,3s)-3-(3-(3- (methoxymethyl)-1- methyl-1H-pyrazole-5- carboxamido)-1H- pyrazol-5- yl)cyclobutyl)methyl isopropylcarbamate | 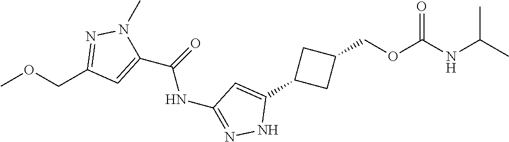 [SMILES: COCc1cc(C(=O)Nc2cc([C@H]3C[C@@H](COC(=O)NC(C)C)C3)[nH]n2)n(C)n1] | ¹H NMR (400 MHz, Chloroform-d) 10.66 (s, 1H), 6.90 (s, 1H), 6.77 (s, 1H), 4.82 (s, 1H), 4.51 (s, 2H), 4.16 (s, 3H), 4.05 (d, J = 5.3 Hz, 2H), 3.84 (d, J = 8.9 Hz, 1H), 3.50 (s, 3H), 3.34 (q, J = 8.9 Hz, 1H), 2.60 (d, J = 12.9 Hz, 1H), 2.50-2.31 (m, 2H), 2.06 (qd, J = 9.6, 2.4 Hz, 2H), 1.18 (d, J = 6.5 Hz, 6H). | 405.30 |
| ((1r,3r)-3-(3-(3- (methoxymethyl)-1- methyl-1H-pyrazole-5- carboxamido)-1H- pyrazol-5- yl)cyclobutyl)methyl isopropylcarbamate | 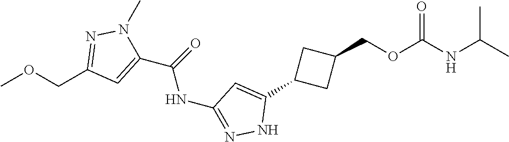 [SMILES: COCc1cc(C(=O)Nc2cc([C@H]3C[C@H](COC(=O)NC(C)C)C3)[nH]n2)n(C)n1] | ¹H NMR (400 MHz, Chloroform-d) 10.88 (s, 1H), 9.96 (s, 1H), 6.93 (s, 1H), 6.82 (s, 1H), 4.57 (s, 1H), 4.52 (s, 2H), 4.15 (s, 5H), 3.83 (d, J = 7.8 Hz, 1H), 3.52 (s, 4H), 2.71 (s, 1H), 2.29 (qd, J = 12.4, 9.0 Hz, 4H), 1.18 (d, J = 6.5 Hz, 6H). | 405.35 |

### Example 4

### 3-fluoro-4-isopropylpyridazine

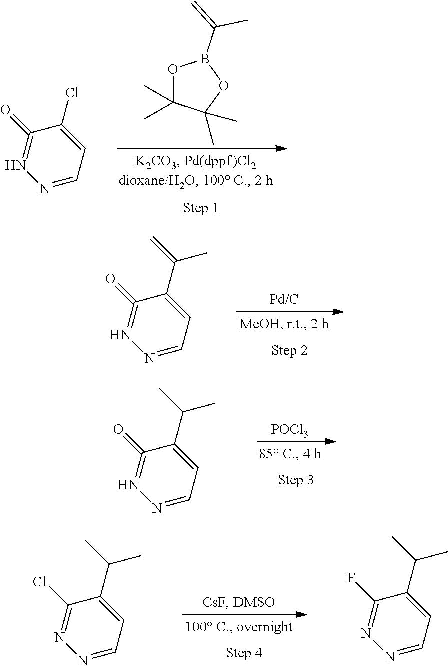

### 4-(prop-1-en-2-yl)pyridazin-3(2H)-one

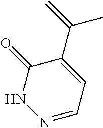

Step 1: A round-bottom flask was charged with 6-chloropyridazin-3(2H)-one (10 g, 1 Eq, 77 mmol), 4,4,5,5-tetramethyl-2-(prop-1-en-2-yl)-1,3,2-dioxaborolane (23 g, 1.8 Eq, 0.14 mol), potassium carbonate (32 g, 3 Eq, 0.23 mol), dioxane/H₂O (20 mL) and a stirbar before being evacuated and purged with nitrogen three times, Pd(dppf)Cl2 (2.8 g, 0.05 Eq, 3.8 mmol) was added. The mixture was stirred at 100° C. for 2 hours. The solution was concentrated. The resulting crude material was purified by flash chromatography (acetonitrile/water). Lyophilization yielded 4-(prop-1-en-2-yl)pyridazin-3-ol (4.0 g, 31%) as a white amorphous solid.

### 4-isopropylpyridazin-3(2H)-one

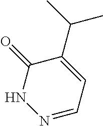

Step 2: A round-bottom flask was charged with 4-(prop-1-en-2-yl)pyridazin-3-ol (4 g, 1 eq, 0.03 mol), Pd/C (0.5 g), MeOH (20 mL) and a stirbar before being evacuated and purged with hydrogen three times. The mixture was stirred at 25° C. for 2 hours. The mixture was filtered, and the filtrate was concentrated to afford 4-isopropylpyridazin-3(2H)-one (3.7 g, 91%) as a yellow oil.

### 3-chloro-4-isopropylpyridazine

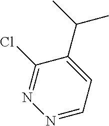

Step 3: A round bottomed flask was charged with 4-isopropylpyridazin-3-ol (3.7 g, 1 eq, 27 mmol), POCl₃ (15 mL) and a stirbar, and the solution was stirred at 85° C. for 4 hours. The reaction mixture was poured into the ice water. The solution was extracted with EA three times. The organic phase was combined and concentrated. The resulting crude material was purified by flash chromatography (acetonitrile/water). Lyophilization yielded 3-chloro-4-isopropylpyridazine (3.8 g, 24 mmol, 91%) as a black oil.

### 3-fluoro-4-isopropylpyridazine

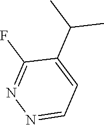

Step 4: A solution of 3-chloro-4-isopropylpyridazine (1.9 g, 12.1 mmol, 1 eq), CsF (12.8 g, 84.7 mmol, 7 eq) and 4A molecular sieves (1 g) in DMSO (25 mL) was stirred at 100° C. for 16 h. The solution was filtered. The solution was purified by FLASH (MeCN/H₂O) to afford 3-fluoro-4-isopropylpyridazine (410 mg, 24%) as a black oil.

### Example 5

### 4-cyclopropylisothiazol-3-ol

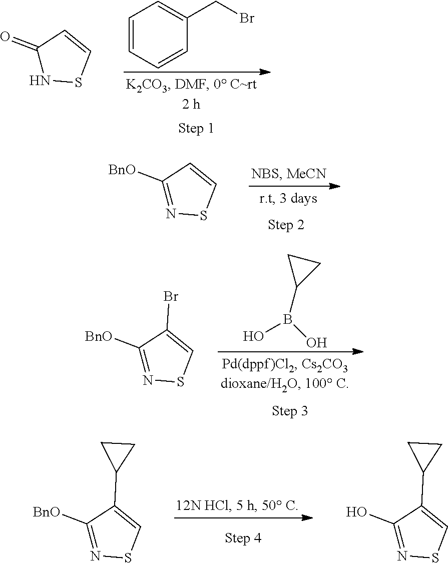

### 3-(benzyloxy)isothiazole

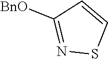 [SMILES: c1ccc(COc2ccsn2)cc1]

Step 1: To a mixture of isothiazol-3(2H)-one (5 g, 0.05 mol) in DMF (50 mL) was added potassium carbonate (13.66 g, 0.1 mol) and (bromomethyl)benzene (10.09 g, 0.06 mol) in portions at 0° C. under nitrogen atmosphere. The mixture was stirred for 4 hours at 25° C. The reaction mixture was diluted with water (20 mL), and the aqueous phase was extracted with EA (20 mL) three times. The combined organic layers were washed with brine three times, dried over sodium sulfate, filtered, and concentrated in vacuo. The crude product was purified by silica gel chromatography (10 g column; eluting with PE/EA; ratio:30/1). Concentration in vacuo resulted in 3-(benzyloxy)isothiazole (5.5 g, 29 mmol, 60%) as a clear oil.

### 3-(benzyloxy)-4-bromoisothiazole

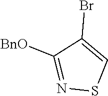 [SMILES: Brc1csnc1OCc1ccccc1]

Step 2: A round bottomed flask was charged with 3-(benzyloxy)isothiazole (5.5 g, 29 mmol), 1-bromopyrrolidine-2,5-dione (5.6 g, 32 mmol), MeCN (60 mL) and a stirbar. The solution was stirred for 2 days at 25° C. The mixture was quenched with water (20 mL), and the aqueous phase was extracted with EA (20 mL) three times. The combined organic layers were washed with brine, dried over sodium sulfate, filtered, and concentrated in vacuo. The crude product was purified by silica gel chromatography (15 g column; eluting with PE/EA; ratio: 50/1). Concentration in vacuo resulted in 3-(benzyloxy)-4-bromoisothiazole (6 g, 0.02 mol, 80%) as a clear oil.

### 3-(benzyloxy)-4-cyclopropylisothiazole

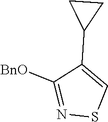 [SMILES: c1ccc(COc2nscc2C2CC2)cc1]

Step 3: A resealable reaction vial was charged with 3-(benzyloxy)-4-bromoisothiazole (200 mg, 740 μmol), cyclopropylboronic acid (636 mg, 7.40 mmol), PdCl₂(dppf) (54.2 mg, 74.0 μmol), Cs₂CO₃ (482 mg, 1.48 mmol), 1,4-dioxane/H₂O (4 mL, 4/1) and a stirbar before being evacuated and purged with nitrogen three times. The mixture was stirred for 12 hours at 100° C. The mixture was diluted with water (20 mL), and the aqueous phase was extracted with EA (20 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 20% to 100% gradient in 20 min; detector, UV 254 nm to afford 3-(benzyloxy)-4-cyclopropylisothiazole (130 mg, 562 μmol, 75.9%) as a brown oil.

### 4-cyclopropylisothiazol-3-ol

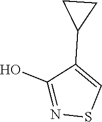

Step 4: A round bottomed flask was charged with 3-(benzyloxy)-4-cyclopropylisothiazole (600 mg, 2.59 mmol) and a stirbar. Conc. HCl (6 mL) was added, and the solution was stirred for 5 hour at 50° C. The mixture was concentrated in vacuo. The residue was purified by reverse flash chromatography with the following conditions: column, C18 silica gel; mobile phase, MeCN in water, 0% to 30% gradient in 10 min; detector, UV 254 nm to afford 4-cyclopropylisothiazol-3-ol (280 mg, 1.98 mmol, 76.5%) as a yellow amorphous solid.

### Example 6

### (1R,3S)-3-(3-(2-(3-methylisoxazol-5-yl)acetamido)-1H-1,2,4-triazol-1-yl)cyclopentyl (1-methylcyclopropyl)carbamate and (1S,3R)-3-(3-(2-(3-methylisoxazol-5-yl)acetamido)-1H-1,2,4-triazol-1-yl)cyclopentyl (1-methylcyclopropyl)carbamate

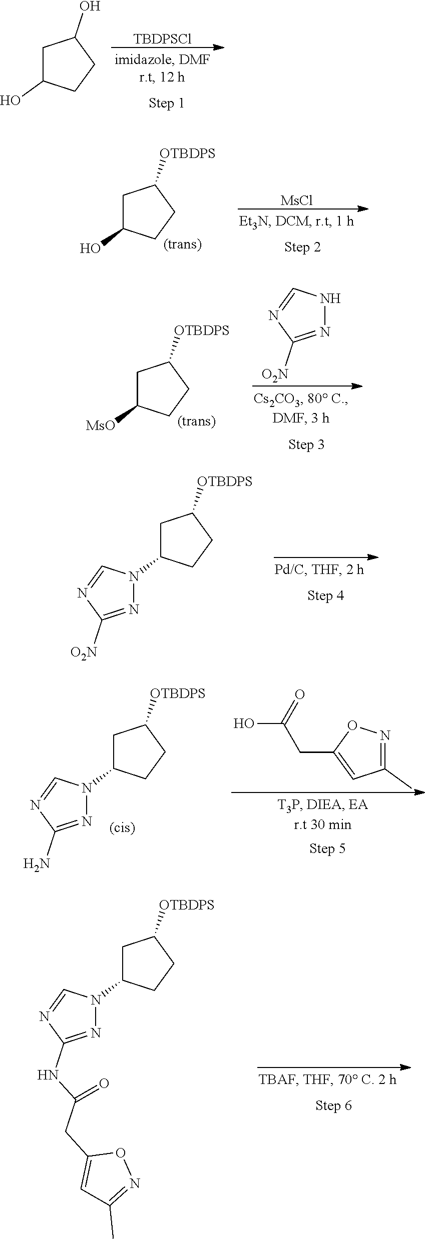

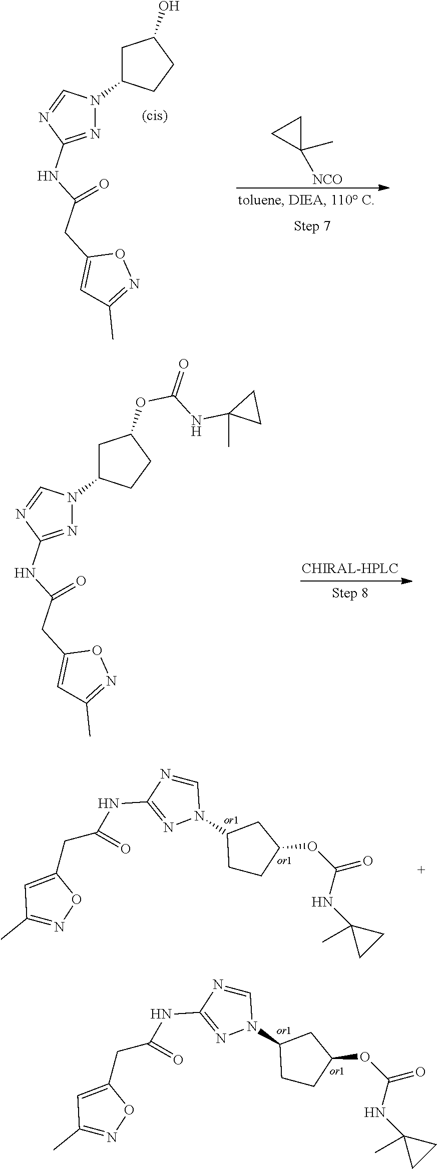

### trans-(1R,3R)-3-((tert-butyldiphenylsilyl)oxy)cyclopentan-1-ol

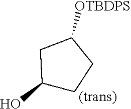

Step 1: To a mixture of cyclopentane-1,3-diol (1 g, 0.01 mol) and imidazole (0.8 g, 0.01 mol)) in DMF (50 mL) was added TBDPSCl (2 g, 9 mmol) in portions at 0° C. under nitrogen atmosphere. The mixture was stirred for 12 h at 25° C. The reaction mixture was diluted with H₂O (50 mL), and the aqueous phase was extracted with EA (100 mL) three times. The combined organic layers were washed with brine, dried over sodium sulfate, filtered, and concentrated in vacuo. The resulting crude material was purified by silica gel chromatography (1 g column; eluting with PE/EA 4/1). Concentration in vacuo resulted in trans-3-((tert-butyldiphenylsilyl)oxy)cyclopentan-1-ol (262 mg, 8%) as a white solid.

m/z (ES⁺) [M+Na]⁺=364.05; HPLC tR=1.373 min.

### trans-(1R,3R)-3-((tert-butyldiphenylsilyl)oxy)cyclopentyl methanesulfonate

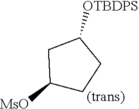

Step 2: To a mixture of trans-3-((tert-butyldiphenylsilyl)oxy)cyclopentan-1-ol (120 mg, 352 μmol) and Et₃N (107 mg, 1.06 mmol) in DCM (3 mL) was added MsCl (63.4 mg, 423 μmol) dropwise at 0° C. under nitrogen atmosphere. The mixture was stirred for 1 h at 25° C. The reaction mixture was diluted with H₂O (50 mL), and the aqueous phase was extracted with EA (100 mL) three times. The combined organic layers were washed with brine, dried over sodium sulfate, filtered, and concentrated in vacuo. The crude product trans-3-((tert-butyldiphenylsilyl)oxy)cyclopentyl methanesulfonate (130 mg, 88.1%) was isolated as a colorless oil.

m/z (ES⁺) [M+H]⁺=419.05; HPLC tR=1.332 min.

### 1-((1S,3R)-3-((tert-butyldiphenylsilyl)oxy)cyclopentyl)-3-nitro-1H-1,2,4-triazole

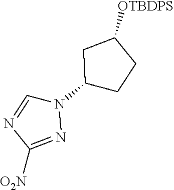

Step 3: To a mixture of 3-nitro-1H-1,2,4-triazole (88 mg, 0.77 mmol) and trans-3-((tert-butyldiphenylsilyl)oxy)cyclopentan-1-ol (0.22 g, 0.64 mmol) in DMF (5 mL) was added Cs₂CO₃ (0.63 g, 1.9 mmol) in portions at 25° C. under nitrogen atmosphere. The mixture was stirred for 3 h at 80° C. The reaction mixture was diluted with H₂O (50 mL), and the aqueous phase was extracted with EA (100 mL) three times. The combined organic layers were washed with brine, dried over sodium sulfate, filtered, and concentrated in vacuo. The resulting crude material was purified by silica gel chromatography (1 g column; eluting with PE/EA=4/1). Concentration in vacuo resulted in 1-(cis-3-((tert-butyldiphenylsilyl)oxy)cyclopentyl)-3-nitro-1H-1,2,4-triazole (16 mg, 5.7%) as a colorless oil.

m/z (ES⁺) [M+H]⁺=437.30; HPLC tR=1.468 min.

### 1-((1S,3R)-3-((tert-butyldiphenylsilyl)oxy)cyclopentyl)-1H-1,2,4-triazol-3-amine

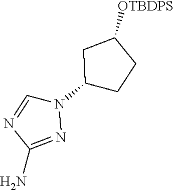

Step 4: A stirred mixture of 1-(cis-3-((tert-butyldiphenylsilyl)oxy)cyclopentyl)-3-nitro-1H-1,2,4-triazole (745 mg, 1.71 mmol) and Pd/C (182 mg) in THE (1 mL) was treated with H₂ for 2 h at 25° C. The solid was filtered out. The filtrate was concentrated under vacuum. Concentration in vacuo resulted in 1-(cis-3-((tert-butyldiphenylsilyl)oxy)cyclopentyl)-1H-1,2,4-triazol-3-amine (625 mg, 90.1%) as a colorless oil.

m/z (ES⁺) [M+H]⁺=407.35; HPLC tR=1.348 min.

### N-(1-((1S,3R)-3-((tert-butyldiphenylsilyl)oxy)cyclopentyl)-1H-1,2,4-triazol-3-yl)-2-(3-methylisoxazol-5-yl)acetamide

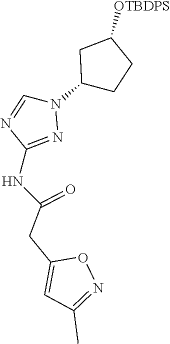

Step 5: To a mixture of 1-(cis-3-((tert-butyldiphenylsilyl)oxy)cyclopentyl)-1H-1,2,4-triazol-3-amine (780 mg, 1.92 mmol), DIEA (744 mg, 5.75 mmol) and 2-(3-methylisoxazol-5-yl)acetic acid (325 mg, 2.30 mmol) in EA (10 mL) was added T₃P (1.83 g, 5.75 mmol) dropwise at 0° C. under nitrogen atmosphere. The mixture was stirred for 30 min at 25° C. The reaction mixture was diluted with H₂O (50 mL), and the aqueous phase was extracted with EA (100 mL) three times. The combined organic layers were washed with brine, dried over sodium sulfate, filtered, and concentrated in vacuo. The crude product was purified by silica gel chromatography (3 g column; eluting with DCM/MeOH; ratio:20/1). Concentration in vacuo resulted in N-(1-(cis-3-((tert-butyldiphenylsilyl)oxy)cyclopentyl)-1H-1,2,4-triazol-3-yl)-2-(3-methylisoxazol-5-yl)acetamide (900 mg, 88.6%) as a yellowish solid.

m/z (ES⁺) [M+H]=530.04; HPLC tR=1.436 min.

### N-(1-((1S,3R)-3-hydroxycyclopentyl)-1H-1,2,4-triazol-3-yl)-2-(3-methylisoxazol-5-yl)acetamide

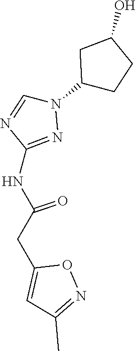

Step 6: A resealable reaction vial was charged with N-(1-(cis-3-((tert-butyldiphenylsilyl)oxy)cyclopentyl)-1H-1,2,4-triazol-3-yl)-2-(3-methylisoxazol-5-yl)acetamide (460 mg, 868 μmol), TBAF (454 mg, 1.74 mmol), THE (5 mL) was added, and a stirbar before being evacuated and purged with nitrogen three times, and the mixture was stirred for 2 h at 70° C. The residue was purified by reverse flash chromatography with the following conditions: column, C18 silica gel; mobile phase, MeCN in water, 10% to 50% gradient in 10 min; detector, UV 254 nm. Concentration in vacuo resulted in N-(1-((cis-3-hydroxycyclopentyl)-1H-1,2,4-triazol-3-yl)-2-(3-methylisoxazol-5-yl)acetamide (80 mg, 32%)) as a colorless oil.

m/z (ES⁺) [M+H]⁺=292.15; HPLC tR=0.743 min.

### (1R,3S)-3-(3-(2-(3-methylisoxazol-5-yl)acetamido)-1H-1,2,4-triazol-1-yl)cyclopentyl (1-methylcyclopropyl)carbamate

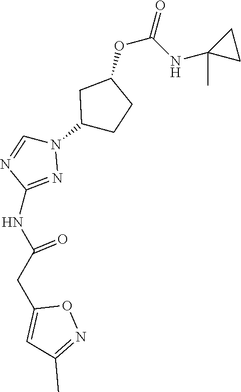

Step 7: A resealable reaction vial was charged with N-(1-(cis-3-hydroxycyclopentyl)-1H-1,2,4-triazol-3-yl)-2-(3-methylisoxazol-5-yl)acetamide (400 mg, 1.37 mmol), 1-isocyanato-1-methylcyclopropane (533 mg, 5.49 mmol), DIEA (710 mg, 5.49 mmol), toluene (10 mL) was added, and a stirbar before being evacuated and purged with nitrogen three times, and the mixture was stirred for 12 h at 110° C. The reaction mixture was diluted with H₂O (50 mL), and the aqueous phase was extracted with EA (100 mL) three times. The combined organic layers were washed with brine, dried over sodium sulfate, filtered, and concentrated in vacuo. The resulting crude material was purified by Pre-HPLC (Column: XBridge Prep OBD C18 Column, 30*150 mm, 5 m; Mobile Phase A: Water(10 mmol/L NH₄HCO₃), Mobile Phase B: ACN; Flow rate: 60 mL/min; Gradient: 10% B to 35% B in 8 min, 35% B; Wave Length: 220 nm; RT1(min): 7.32). Lyophilization cis-3-(3-(2-(3-methylisoxazol-5-yl)acetamido)-1H-1,2,4-triazol-1-yl)cyclopentyl (1-methylcyclopropyl)carbamate (100 mg, 18.7%) as a white solid.

### (1R,3S)-3-(3-(2-(3-methylisoxazol-5-yl)acetamido)-1H-1,2,4-triazol-1-yl)cyclopentyl (1-methylcyclopropyl)carbamate and (1S,3R)-3-(3-(2-(3-methylisoxazol-5-yl)acetamido)-1H-1,2,4-triazol-1-yl)cyclopentyl (1-methylcyclopropyl)carbamate

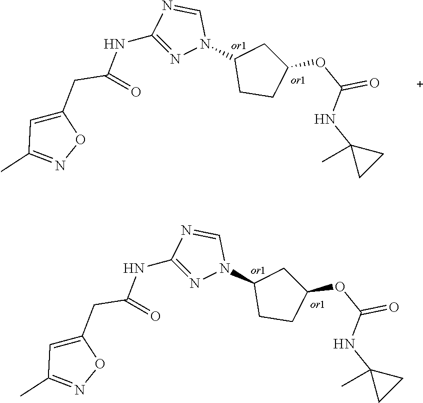

Step 8: Cis-3-(3-(2-(3-methylisoxazol-5-yl)acetamido)-1H-1,2,4-triazol-1-yl)cyclopentyl (1-methyl cyclopropyl)carbamate (150 mg, 386 μmol) was purified by chiral Pre-HPLC (Column: DZ-CHIRALPAK IG-3, 4.6*50 mm, 3.0 m; Mobile Phase A: Hex(0.2% IPAmine): (EtOH: DCM=1:1)=60:40; Flow rate: 1 mL/min; Injection Volume: Sul mL). Lyophilization yielded (1R,3S)-3-(3-(2-(3-methylisoxazol-5-yl)acetamido)-1H-1,2,4-triazol-1-yl)cyclopentyl (1-methylcyclopropyl)carbamate (38.8 mg, 25.9%) as a white solid.

m/z (ES⁺) [M+H]⁺=389.15; HPLC tR=1.082 min.

¹H NMR (400 MHz, DMSO-d₆) δ 10.68 (s, 1H), 8.41 (s, 1H), 7.41 (s, 1H), 6.24 (s, 1H), 4.99 (s, 1H), 4.76-4.68 (m, 1H), 3.87 (s, 2H), 2.60-2.52 (m, 1H), 2.08 (d, J=24.8 Hz, 3H), 2.03-1.89 (m, 4H), 1.81 (s, 1H), 1.23 (s, 2H), 0.59 (s, 2H), 0.47 (s, 2H).

Cis-3-(3-(2-(3-methylisoxazol-5-yl)acetamido)-1H-1,2,4-triazol-1-yl)cyclopentyl (1-methyl cyclopropyl)carbamate (150 mg, 386 μmol) was purified by chiral Pre-HPLC (Column: DZ-CHIRALPAK IG-3, 4.6*50 mm, 3.0 m; Mobile Phase A: Hex(0.2% IPAmine): (EtOH: DCM=1:1)=60:40; Flow rate: 1 mL/min; Injection Volume: 5 ul mL). Lyophilization yielded (1S,3R)-3-(3-(2-(3-methylisoxazol-5-yl)acetamido)-1H-1,2,4-triazol-1-yl)cyclopentyl (1-methylcyclopropyl)carbamate (35.1 mg, 90.4 μmol, 23.4%) as a white solid. m/z (ES⁺) [M+H]⁺=389.15; HPLC tR=1.082 min.

¹H NMR (400 MHz, DMSO-d₆) δ 10.69 (s, 1H), 8.41 (s, 1H), 7.41 (s, 1H), 6.24 (s, 1H), 4.99 (s, 1H), 4.71 (q, J=7.6 Hz, 1H), 3.87 (s, 2H), 2.60-2.52 (m, 1H), 2.21 (s, 3H), 2.11 (s, 1H), 1.95 (s, 2H), 1.80 (s, 1H), 1.23 (s, 3H), 0.59 (s, 2H), 0.47 (s, 2H).

### Example 7

### (1s,4s)-N-isopropyl-4-(3-(2-(3-methylisoxazol-5-yl)acetamido)-1H-pyrazol-5-yl)cyclohexane-1-carboxamide and (1r,4r)-N-isopropyl-4-(3-(2-(3-methylisoxazol-5-yl)acetamido)-1H-pyrazol-5-yl)cyclohexane-1-carboxamide

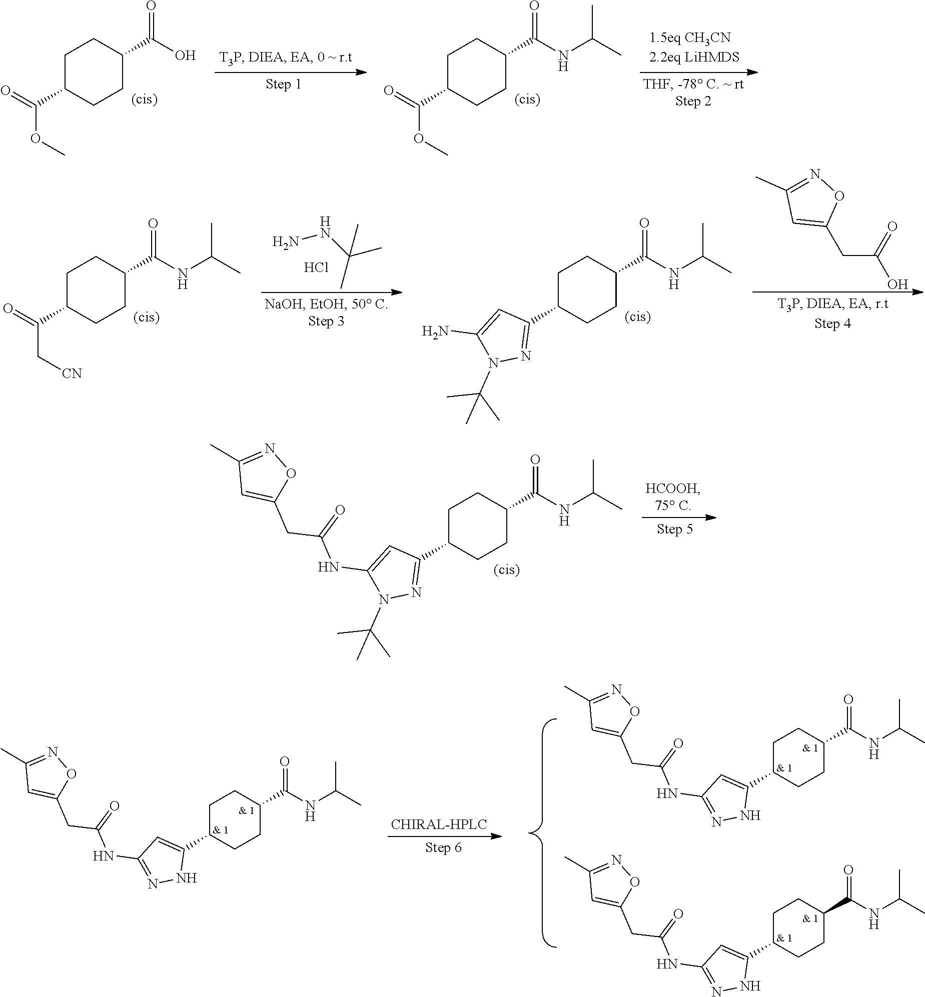

### methyl (1s,4s)-4-(isopropylcarbamoyl)cyclohexane-1-carboxylate

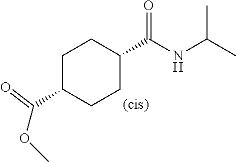 [SMILES: COC(=O)[C@H]1CC[C@@H](C(=O)NC(C)C)CC1]

Step 1: To a stirred solution of (1s,4s)-4-(methoxycarbonyl)cyclohexane-1-carboxylic acid (3 g, 0.02 mol) and propan-2-amine (1 g, 0.02 mol) in ethyl acetate (20 mL) was added DIEA (6 g, 0.05 mol) and T3P (12 g, 50% Wt in ethyl acetate, 0.02 mmol). The reaction was stirred at room temperature for 1 hour. LCMS showed the reaction was completed. The mixture was concentrated and recrystallized from PE/EA=1:8. This resulted in methyl (1s,4s)-4-(isopropylcarbamoyl)cyclohexane-1-carboxylate (3.24 g, 14.3 mmol, 90%) as white solid.

m/z (ES⁺) [M+H]⁺=228.20; HPLC tR=0.918 min.

### (1s,4s)-4-(2-cyanoacetyl)-N-isopropylcyclohexane-1-carboxamide

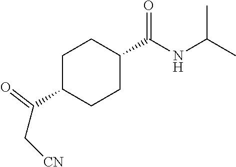 [SMILES: CC(C)NC(=O)[C@H]1CC[C@@H](C(=O)CC#N)CC1]

Step 2: To a solution of methyl (1s,4s)-4-(isopropylcarbamoyl)cyclohexane-1-carboxylate (2.2 g, 9.7 mmol) and acetonitrile (0.60 g, 15 mmol) in THE (20 mL) was added LiHMDS (21 mL, 1M in THF, 21 mmol) dropwise at −78° C. under nitrogen atmosphere. The mixture was warmed to 25° C. and stirred for 4 hours. The mixture was quenched with saturated NH₄Cl. The reaction mixture was diluted with water (20 mL), and the aqueous phase was extracted with EA (20 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 10% to 70% gradient in 15 min; detector UV 254 nm to (1s,4s)-4-(2-cyanoacetyl)-N-isopropylcyclohexane-1-carboxamide (1.75 g, 7.41 mmol, 77%) as an off-white amorphous solid.

m/z (ES⁺) [M+H]⁺=237.20; HPLC tR=0.739 min.

### (1s,4s)-4-(5-amino-1-(tert-butyl)-1H-pyrazol-3-yl)-N-isopropylcyclohexane-1-carboxamide

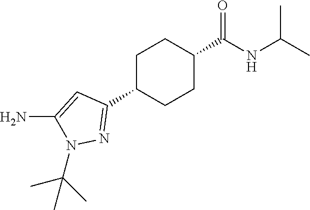 [SMILES: CC(C)NC(=O)[C@H]1CC[C@@H](c2cc(N)n(C(C)(C)C)n2)CC1]

Step 3: Sodium hydroxide (439 mg, 11.0 mmol) was added in portions to a suspension of tert-butylhydrazine hydrochloride (1.37 g, 11.0 mmol) in EtOH (14 mL) at room temperature, and stirred at room temperature for 1 hour. A solution of (1s,4s)-4-(2-cyanoacetyl)-N-isopropylcyclohexane-1-carboxamide (1.73 g, 7.32 mmol) in ethanol was added at room temperature, then the mixture was heated to 50° C. internal and stirred overnight. The mixture was quenched with water (20 mL), and the aqueous phase was extracted with EA (20 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 10% to 60% gradient in 15 min; detector, UV 254 nm to afford (1s,4s)-4-(3-amino-1-(tert-butyl)-1H-pyrazol-5-yl)-N-isopropylcyclohexane-1-carboxamide (1.3 g, 4.2 mmol, 58%) as a white amorphous solid.

m/z (ES⁺) [M+H]⁺=307.25; HPLC tR=0.788 min.

### (1s,4s)-4-(1-(tert-butyl)-5-(2-(3-methylisoxazol-5-yl)acetamido)-1H-pyrazol-3-yl)-N-isopropylcyclohexane-1-carboxamide

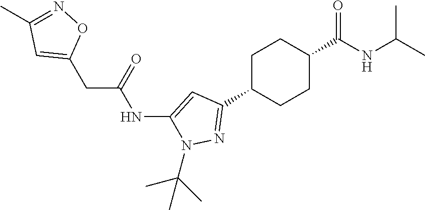 [SMILES: Cc1cc(CC(=O)Nc2cc([C@H]3CC[C@@H](C(=O)NC(C)C)CC3)nn2C(C)(C)C)on1]

Step 4: To a mixture of (1s,4s)-4-(3-amino-1-(tert-butyl)-1H-pyrazol-5-yl)-N-isopropylcyclohexane-1-carboxamide (1.4 g, 4.6 mmol), 2-(3-methylisoxazol-5-yl)acetic acid (0.97 g, 6.9 mmol) and DIEA (1.8 g, 2.4 mL, 14 mmol) in EA (15 mL) was added phosphane-t3 in EA (4.4 g, 50% wt in EA, 6.9 mmol) dropwise at 0° C. under nitrogen atmosphere. The mixture was stirred for 2 hours at 25° C. The mixture was quenched with water (20 mL), and the aqueous phase was extracted with EA (20 mL) three times. The combined organic layers were washed with brine, dried over sodium sulfate, filtered, and concentrated in vacuo. The residue was purified by reverse phase flash chromatography with the following conditions: column, C18 silica gel; mobile phase, MeCN in water, 20% to 80% gradient in 20 min; detector, UV 220 nm to afford (1s,4s)-4-(1-(tert-butyl)-3-(2-(3-methylisoxazol-5-yl)acetamido)-1H-pyrazol-5-yl)-N-isopropylcyclohexane-1-carboxamide (1.8 g, 4.2 mmol, 92%) as a yellow amorphous solid.

m/z (ES⁺) [M+H]⁺=430.45; HPLC tR=0.955 min.

### (1s,4s)-N-isopropyl-4-(3-(2-(3-methylisoxazol-5-yl)acetamido)-1H-pyrazol-5-yl)cyclohexane-1-carboxamide

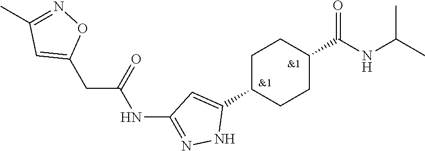 [SMILES: Cc1cc(CC(=O)Nc2cc([C@H]3CC[C@@H](C(=O)NC(C)C)CC3)[nH]n2)on1]

Step 5: A round bottomed flask was charged with (1s,4s)-4-(1-(tert-butyl)-3-(2-(3-methylisoxazol-5-yl)acetamido)-1H-pyrazol-5-yl)-N-isopropylcyclohexane-1-carboxamide (200 mg, 466 μmol), and a stirbar. HCOOH (3 mL) was added, and the solution was stirred for 12 hours at 75° C. The mixture was concentrated in vacuo. The residue was purified by reverse phase flash chromatography with the following conditions: column, C18 silica gel; mobile phase, MeCN in water, 10% to 60% gradient in 15 min; detector, UV 254 nm to afford (1s,4s)-N-isopropyl-4-(3-(2-(3-methylisoxazol-5-yl)acetamido)-1H-pyrazol-5-yl)cyclohexane-1-carboxamide (120 mg, 321 μmol, 69.0%) as a white amorphous solid.

m/z (ES⁺) [M+H]⁺=374.10; HPLC tR=0.646 min.

### (1s,4s)-N-isopropyl-4-(3-(2-(3-methylisoxazol-5-yl)acetamido)-1H-pyrazol-5-yl)cyclohexane-1-carboxamide and (1r,4r)-N-isopropyl-4-(3-(2-(3-methylisoxazol-5-yl)acetamido)-1H-pyrazol-5-yl)cyclohexane-1-carboxamide

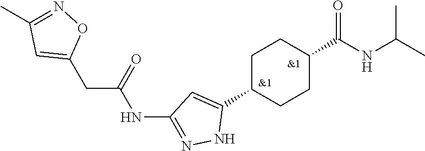 [SMILES: Cc1cc(CC(=O)Nc2cc([C@H]3CC[C@@H](C(=O)NC(C)C)CC3)[nH]n2)on1]

(1s,4s)-N-isopropyl-4-(3-(2-(3-methylisoxazol-5-yl)acetamido)-1H-pyrazol-5-yl)cyclohexane-1-carboxamide (120 mg, 321 μmol) was purified by chiral Pre-HPLC (Column: CHIRAL ART Amylose-SA, 2*25 cm, 5 m; Mobile Phase A: MtBE(0.1% DEA)-HPLC, Mobile Phase B: EtOH—HPLC; Flow rate: 20 mL/min; Gradient: 50% B to 50% B in 21 min; Wave Length: 220/254 nm; RT1(min): 5.48; Sample Solvent: EtOH—HPLC; Injection Volume: 1 mL; Number Of Runs: 4). Lyophilization yielded (1s,4s)-N-isopropyl-4-(3-(2-(3-methylisoxazol-5-yl)acetamido)-1H-pyrazol-5-yl)cyclohexane-1-carboxamide (64.3 mg, 172 μmol, 53.6%) as a white amorphous solid.

m/z (ES⁺) [M+H]⁺=374.10; HPLC tR=0.658 min.

¹H NMR (400 MHz, DMSO-d₆) δ 12.05 (s, 1H), 10.63 (s, 1H), 7.48 (d, J=7.8 Hz, 1H), 6.30 (s, 1H), 6.23 (s, 1H), 3.91-3.72 (m, 3H), 2.82 (s, 1H), 2.20 (s, 4H), 1.93 (d, J=15.1 Hz, 2H), 1.65 (t, J=9.0 Hz, 4H), 1.49 (q, J=6.4, 4.2 Hz, 2H), 1.01 (d, J=6.6 Hz, 6H).

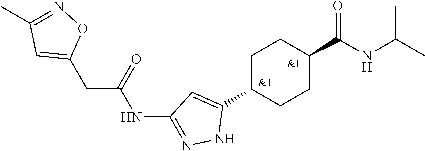 [SMILES: Cc1cc(CC(=O)Nc2cc([C@H]3CC[C@H](C(=O)NC(C)C)CC3)[nH]n2)on1]

(1r,4r)-N-isopropyl-4-(3-(2-(3-methylisoxazol-5-yl)acetamido)-1H-pyrazol-5-yl)cyclohexane-1-carboxamide (120 mg, 321 μmol) was purified by chiral Pre-HPLC (Column: CHIRAL ART Amylose-SA, 2*25 cm, 5 m; Mobile Phase A: MtBE(0.1% DEA)-HPLC, Mobile Phase B: EtOH—HPLC; Flow rate: 20 mL/min; Gradient: 50% B to 50% B in 21 min; Wave Length: 220/254 nm; RT2(min): 11.01; Sample Solvent: EtOH—HPLC; Injection Volume: 1 mL; Number Of Runs: 4). Lyophilization yielded (1r,4r)-N-isopropyl-4-(3-(2-(3-methylisoxazol-5-yl)acetamido)-1H-pyrazol-5-yl)cyclohexane-1-carboxamide (19.9 mg, 53.3 μmol, 16.6%) as a white amorphous solid.

m/z (ES⁺) [M+H]⁺=374.15; HPLC tR=0.647 min.

¹H NMR (400 MHz, DMSO-d₆) δ 12.08 (s, 1H), 10.61 (s, 1H), 7.58 (d, J=7.7 Hz, 1H), 6.24 (d, J=11.9 Hz, 2H), 3.90-3.70 (m, 3H), 2.61-2.52 (m, 1H), 2.20 (s, 3H), 2.07 (tt, J=11.7, 3.4 Hz, 1H), 1.96 (dd, J=13.2, 3.5 Hz, 2H), 1.76 (dd, J=13.5, 3.5 Hz, 2H), 1.46 (qd, J=12.7, 2.9 Hz, 2H), 1.38-1.22 (m, 2H), 1.03 (d, J=6.6 Hz, 6H).

### Example 8

### (1s,4s)-4-(3-(2-(3-methylisoxazol-5-yl)acetamido)-1H-pyrazol-5-yl)cyclohexyl isopropylcarbamate

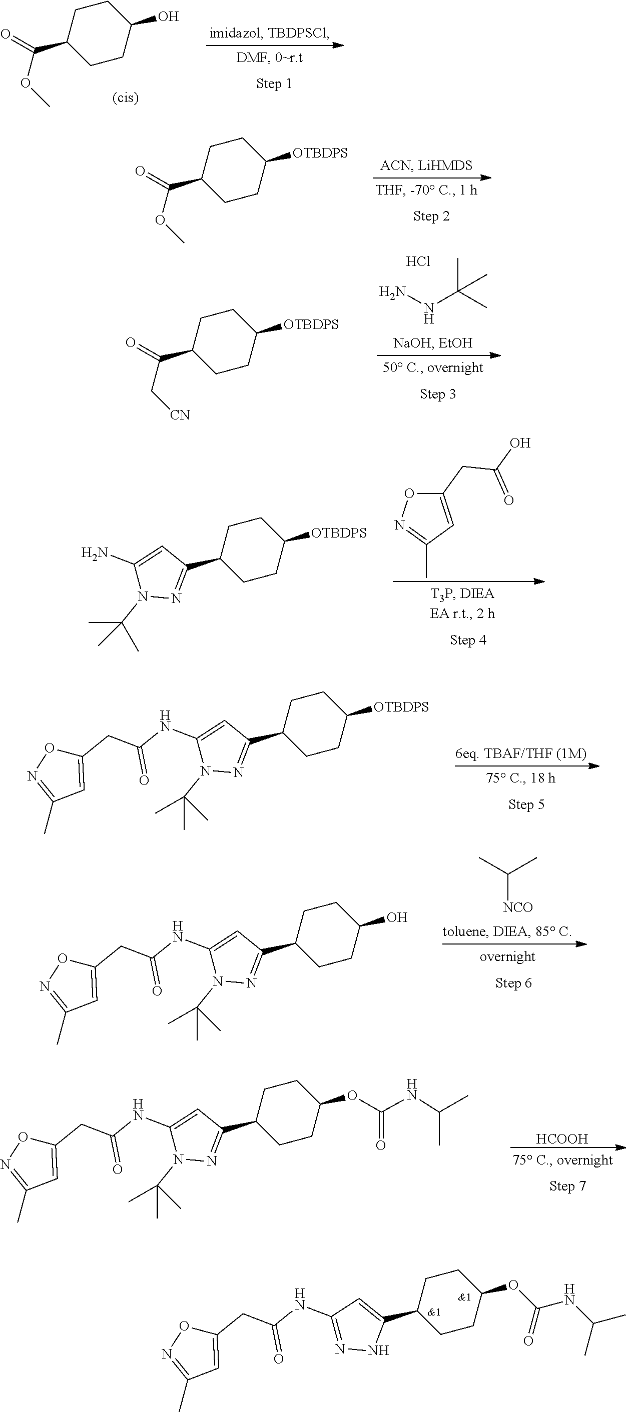

### methyl (1s,4s)-4-((tert-butyldiphenylsilyl)oxy)cyclohexane-1-carboxylate

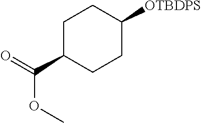

Step 1: To a stirred solution of methyl (1s,4s)-4-hydroxycyclohexane-1-carboxylate (5 g, 0.03 mol) in DMF (25 mL) was added imidazole (6 g, 0.09 mol) and TBDPS-Cl (0.01 kg, 0.04 mol) at 0° C. The reaction was stirred for overnight at room temperature. The resulting solution was diluted with 30 ml of water, then extracted with 3×40 mL of ethyl acetate. The organic layers were combined, washed with saturated brine, dried over anhydrous sodium sulfate and concentrated under vacuum. The residue was purified by silica gel column chromatography with petroleum ether to give methyl (1s,4s)-4-((tert-butyldiphenylsilyl)oxy)cyclohexane-1-carboxylate (10.59 g, 26.70 mmol, 80%) as a colorless oil. m/z (ES⁺) [M+H]⁺=397.30; HPLC tR=L⁵⁹⁵ min.

### 3-((1s,4s)-4-((tert-butyldiphenylsilyl)oxy)cyclohexyl)-3-oxopropanenitrile

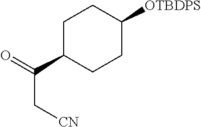

Step 2: To a stirred solution of methyl (1s,4s)-4-((tert-butyldiphenylsilyl)oxy)cyclohexane-1-carboxylate (5 g, 0.01 mol) in Tetrahydrofuran (15 mL) was added acetonitrile (1.0 g, 25 mmol). The mixture was added LiHMDS (25 mL of a 1M solution in THF, 0.03 mol) dropwise at −70° C. under N₂. The reaction was stirred at room temperature for 1 hour. The reaction was quenched with saturated ammonium chloride solution, and extracted with 3×40 mL of ethyl acetate. The organic layers were combined, washed with brine, dried and concentrated under vacuum. The residue was purified by silica gel column chromatography with petroleum ether/ethyl acetate/(5/1) to give 3-((1s,4s)-4-((tert-butyldiphenylsilyl)oxy)cyclohexyl)-3-oxopropanenitrile (4.6 g, 11 mmol, 90%) as a yellow oil.

m/z (ES⁺) [M+H]⁺=406.10; HPLC tR=1.386 min.

### 1-(tert-butyl)-3-((1s,4s)-4-((tert-butyldiphenylsilyl)oxy)cyclohexyl)-1H-pyrazol-5-amine

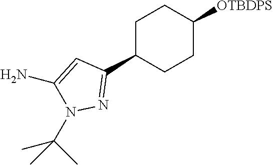

Step 3: A solution of tert-butylhydrazine hydrochloride (2.1 g, 17 mmol) and NaOH (0.67 g, 17 mmol) in ethanol (25 mL) was stirred for 1 h, then 3-((1s,4s)-4-((tert-butyldiphenylsilyl)oxy)cyclohexyl)-3-oxopropanenitrile (4.5 g, 11 mmol) was added. The reaction was stirred at 50° C. overnight. The solid was filtered out. The filtrate was concentrated under vacuum. The residue was purified by reverse flash chromatography with the following conditions: column, C18 silica gel; mobile phase, ACN in water, 95% gradient in 10 min; detector, UV 220 nm to afford 1-(tert-butyl)-5-((1s,4s)-4-((tert-butyldiphenylsilyl)oxy)cyclohexyl)-1H-pyrazol-3-amine (2.5 g, 5.3 mmol, 47%) as a yellow oil.

m/z (ES⁺) [M+H]⁺=476.35; HPLC tR=1L²³⁴ min.

### N-(1-(tert-butyl)-3-((1s,4s)-4-((tert-butyldiphenylsilyl)oxy)cyclohexyl)-1H-pyrazol-5-yl)-2-(3-methylisoxazol-5-yl)acetamide

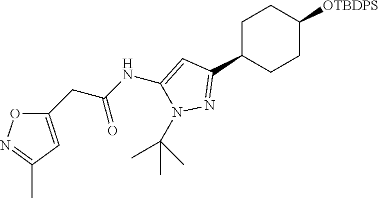

Step 4: To a stirred solution of 1-(tert-butyl)-5-((1s,4s)-4-((tert-butyldiphenylsilyl)oxy)cyclohexyl)-1H-pyrazol-3-amine (1.9 g, 1 eq, 4.0 mmol) and 2-(3-methylisoxazol-5-yl)acetic acid (0.85 g, 1.5 eq, 6.0 mmol) in ethyl acetate (15 mL) was added DIEA (1.5 g, 2.1 mL, 3 eq, 12 mmol) and propanephosphonic acid cyclic anhydride/EA (3.8 g, 50% wt, 6.0 mmol). The reaction was stirred at room temperature for 2 hour. LCMS showed the reaction was complete. The mixture was concentrated and purified by reverse flash chromatography with the following conditions: column, C18 silica gel; mobile phase, ACN in water, 95% to 100% gradient in 10 min; detector, UV 220 nm. This resulted in N-(1-(tert-butyl)-5-((1s,4s)-4-((tert-butyldiphenylsilyl)oxy)cyclohexyl)-1H-pyrazol-3-yl)-2-(3-methylisoxazol-5-yl)acetamide (2.043 g, 3.411 mmol, 85%) as a yellow oil.

m/z (ES⁺) [M+H]⁺=599.55; HPLC tR=1.596 min.

### N-(1-(tert-butyl)-3-((1s,4s)-4-hydroxycyclohexyl)-1H-pyrazol-5-yl)-2-(3-methylisoxazol-5-yl)acetamide

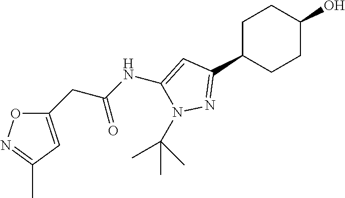 [SMILES: Cc1cc(CC(=O)Nc2cc([C@H]3CC[C@@H](O)CC3)nn2C(C)(C)C)on1]

Step 5: To a stirred solution of N-(1-(tert-butyl)-5-((1s,4s)-4-((tert-butyldiphenylsilyl)oxy)cyclohexyl)-1H-pyrazol-3-yl)-2-(3-methylisoxazol-5-yl)acetamide (1.9 g, 3.2 mmol) in THE (15 mL) was added 1M TBAF in THF (19 mL). The reaction was stirred at 75° C. for 18 h. The resulting mixture was washed with water and extracted with ethyl acetate. The organic layers were combined, dried and concentrated under vacuum. The mixture was purified by reverse flash chromatography with the following conditions: column, C18 silica gel; mobile phase, ACN in water, 19% gradient in 10 min; detector, UV 220 nm to afford N-(1-(tert-butyl)-5-((1s,4s)-4-hydroxycyclohexyl)-1H-pyrazol-3-yl)-2-(3-methylisoxazol-5-yl)acetamide (880 mg, 2.44 mmol, 77%) as a yellow oil.

m/z (ES⁺) [M+H]⁺=361.10; HPLC tR=0.858 min.

### (1s,4s)-4-(1-(tert-butyl)-5-(2-(3-methylisoxazol-5-yl)acetamido)-1H-pyrazol-3-yl)cyclohexyl isopropylcarbamate

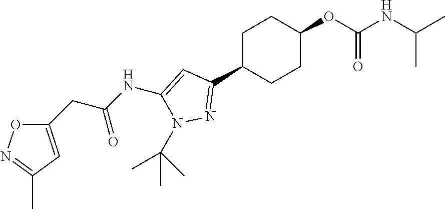 [SMILES: Cc1cc(CC(=O)Nc2cc([C@H]3CC[C@@H](OC(=O)NC(C)C)CC3)nn2C(C)(C)C)on1]

Step 6: To a stirred solution of N-(1-(tert-butyl)-5-((1s,4s)-4-hydroxycyclohexyl)-1H-pyrazol-3-yl)-2-(3-methylisoxazol-5-yl)acetamide (650 mg, 1.80 mmol) in toluene (6 mL) was added DIEA (699 mg, 5.41 mmol) and 2-isocyanatopropane (460 mg, 5.41 mmol). The reaction was stirred at 85° C. for overnight. The resulting mixture was concentrated and purified by reverse flash chromatography with the following conditions: column, C18 silica gel; mobile phase, ACN in water, 19% gradient in 10 min; detector, UV 220 nm to afford (1s,4s)-4-(1-(tert-butyl)-3-(2-(3-methylisoxazol-5-yl)acetamido)-1H-pyrazol-5-yl)cyclohexyl isopropylcarbamate (368 mg, 826 μmol, 45.8%), as a yellow oil.

m/z (ES⁺) [M+H]⁺=446.15; HPLC tR=1.059 min.

### (1s,4s)-4-(3-(2-(3-methylisoxazol-5-yl)acetamido)-1H-pyrazol-5-yl)cyclohexyl isopropylcarbamate

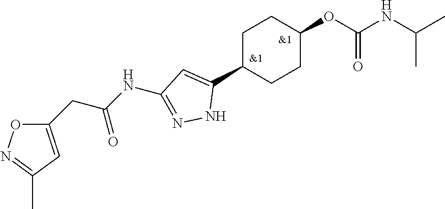 [SMILES: Cc1cc(CC(=O)Nc2cc([C@H]3CC[C@@H](OC(=O)NC(C)C)CC3)[nH]n2)on1]

Step 7: (1s,4s)-4-(1-(tert-butyl)-3-(2-(3-methylisoxazol-5-yl)acetamido)-1H-pyrazol-5-yl)cyclohexyl isopropylcarbamate (350 mg, 786 μmol) was dissolved in formic acid (6 mL). The reaction was stirred at 75° C. for overnight. The mixture was concentrated and purified by reverse flash chromatography with the following conditions: column, C18 silica gel; mobile phase, ACN in water, 46% gradient in 10 min; detector, UV 220 nm to afford (1s,4s)-4-(3-(2-(3-methylisoxazol-5-yl)acetamido)-1H-pyrazol-5-yl)cyclohexyl isopropylcarbamate (210 mg, 539 μmol, 68.6%) as a white solid. The solid was purified by Prep-HPLC (Column: Xselect CSH OBD Column 30*150 mm, Sum; Mobile Phase A: Water (0.1% FA), Mobile Phase B: ACN; Flow rate: 60 mL/min; Gradient: 30% B to 31% B in 7 min, 31% B; Wave Length: 220 nm). Lyophilization yielded (1s,4s)-4-(3-(2-(3-methylisoxazol-5-yl)acetamido)-1H-pyrazol-5-yl)cyclohexyl isopropylcarbamate (139.2 mg, 357.4 μmol, 69.6%) as a white amorphous solid.

m/z (ES⁺) [M+H]⁺=390.15; HPLC tR=0.726 min.

¹H NMR (400 MHz, DMSO-d₆) δ 12.11 (s, 1H), 10.65 (s, 1H), 6.94 (d, J=7.6 Hz, 1H), 6.29 (s, 1H), 6.22 (s, 1H), 4.75 (s, 1H), 3.83 (s, 2H), 3.61-3.55 (m, 1H), 2.67 (s, 1H), 2.20 (s, 3H), 1.78-1.71 (m, 5H), 1.64 (t, J=14.2 Hz, 3H), 1.04 (d, J=6.5 Hz, 6H).

### Example 9

### N-(5-((1S,3R)-3-(4,4-dimethyl-2-oxoimidazolidin-1-yl)cyclopentyl)-1H-pyrazol-3-yl)-2-(3-methylisoxazol-5-yl)acetamide and N-(5-((1R,3S)-3-(4,4-dimethyl-2-oxoimidazolidin-1-yl)cyclopentyl)-1H-pyrazol-3-yl)-2-(3-methylisoxazol-5-yl)acetamide

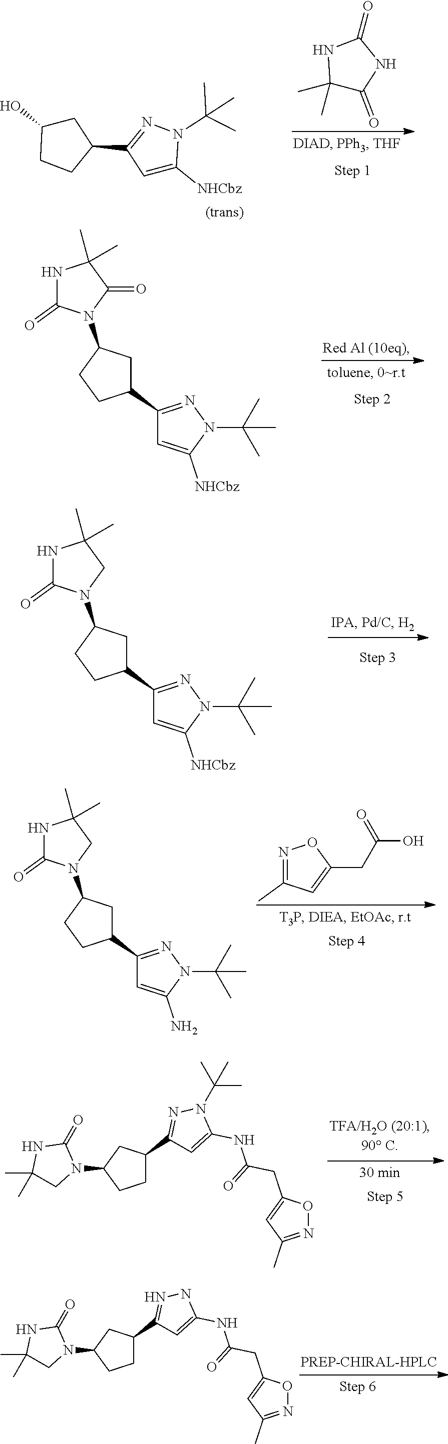

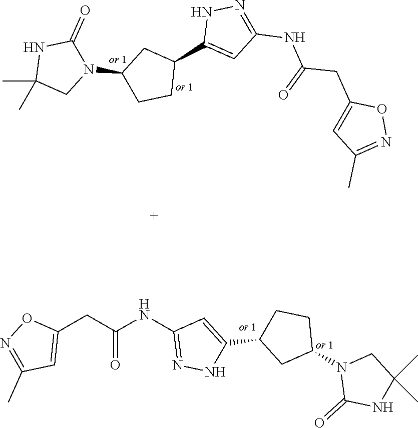 [SMILES: Cc1cc(CC(=O)Nc2cc([C@H]3CC[C@@H](N4CC(C)(C)NC4=O)C3)[nH]n2)on1 ; Cc1cc(CC(=O)Nc2cc([C@@H]3CC[C@H](N4CC(C)(C)NC4=O)C3)[nH]n2)on1]

### benzyl (1-(tert-butyl)-3-((1R,3R)-3-(4,4-dimethyl-2,5-dioxoimidazolidin-1-yl)cyclopentyl)-1H-pyrazol-5-yl)carbamate

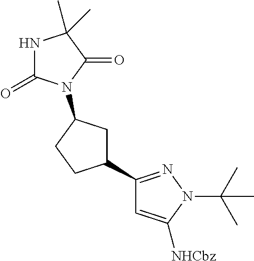

Step 1: To an ice cooled solution of benzyl (1-(tert-butyl)-5-(trans-3-hydroxycyclopentyl)-1H-pyrazol-3-yl)carbamate (1 g, 3 mmol), 5,5-dimethylimidazolidine-2,4-dione (0.4 g, 3 mmol) and triphenylphosphine (1 g, 4 mmol) in THE (10 mL), DIAD (0.8 g, 4 mmol) was added, and then the resulting mixture was stirred at room temperature for 2 hours. The resulting mixture was diluted with EA (100 mL), and washed with brine (2*80 mL), dried over Na₂SO₄. The organic layers was concentrated under vacuum. The residue was purified by reverse flash chromatography with the following conditions: column, C18 silica gel; mobile phase, MeCN in water, 0% to 100% gradient in 40 min; detector, UV 220 nm to afford benzyl (1-(tert-butyl)-5-(cis-3-(4,4-dimethyl-2,5-dioxoimidazolidin-1-yl)cyclopentyl)-1H-pyrazol-3-yl)carbamate (1.03 g, 2.20 mmol, 80%) as a white solid.

m/z (ES⁺) [M+H]⁺=468.25; HPLC tR=1.106 min.

### benzyl (1-(tert-butyl)-3-((1R,3R)-3-(4,4-dimethyl-2-oxoimidazolidin-1-yl)cyclopentyl)-1H-pyrazol-5-yl)carbamate

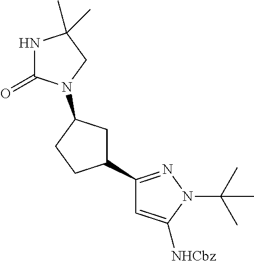

Step 2: To a solution of benzyl (1-(tert-butyl)-5-(cis-3-(4,4-dimethyl-2,5-dioxoimidazolidin-1-yl)cyclopentyl)-1H-pyrazol-3-yl)carbamate (100 mg, 1.07 mmol) in toluene (5 mL) at 0° C., a solution of Red-Al (3.09 g, 70% wt in toluene) was added dropwise, slowly enough to maintain the internal temperature below 5° C. After stirring for an additional 5 hours at room temperature, the resulting mixture was quenched with NH₄Cl (sat.aq. 150 mL), and extracted with EA (3*150 mL). The combined organic layers were washed with brine (2*100 mL), dried over Na₂SO₄ and concentrated under vacuum. This product was combined from five more identically prepared batches (each starting with 100 mg), and purified by reverse flash chromatography with the following conditions: column, C18 silica gel; mobile phase, MeCN in water, 0% to 100% gradient in 15 min; detector, UV 220 nm to afford benzyl (1-(tert-butyl)-5-(cis-3-(4,4-dimethyl-2-oxoimidazolidin-1-yl)cyclopentyl)-1H-pyrazol-3-yl)carbamate (250 mg, 551 μmol, 51.5%) as a white solid.

m/z (ES⁺) [M+H]⁺=454.15; HPLC tR=1.103 min.

### 1-((1R,3R)-3-(5-amino-1-(tert-butyl)-1H-pyrazol-3-yl)cyclopentyl)-4,4-dimethylimidazolidin-2-one

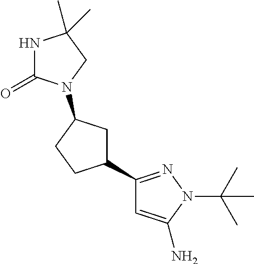

Step 3: At room temperature (20-25° C.) a suspension of Pd/C (50%, 77 mg) and benzyl (1-(tert-butyl)-5-(cis-3-(4,4-dimethyl-2-oxoimidazolidin-1-yl)cyclopentyl)-1H-pyrazol-3-yl)carbamate (330 mg, 728 μmol) in 2-propanol (10 mL) was degassed and purged with hydrogen (3 cycles), then stirred at room temperature under a hydrogen balloon for 0.5 hours. The suspension was filtered. The filtrate was concentrated under vacuum and the residue was purified by reverse phase flash chromatography with the following conditions: column, C18 silica gel; mobile phase, MeCN in water, 0% to 100% gradient in 20 min; detector, UV 220 nm to give 1-(cis-3-(3-amino-1-(tert-butyl)-1H-pyrazol-5-yl)cyclopentyl)-4,4-dimethylimidazolidin-2-one (180 mg, 563 μmol, 77.4%) as a white solid.

m/z (ES⁺) [M+H]⁺=320.25; HPLC tR=1.093 min.

### N-(1-(tert-butyl)-3-((1S,3R)-3-(4,4-dimethyl-2-oxoimidazolidin-1-yl)cyclopentyl)-1H-pyrazol-5-yl)-2-(3-methylisoxazol-5-yl)acetamide

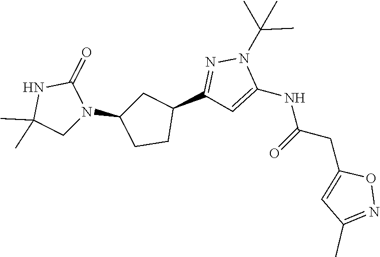 [SMILES: Cc1cc(CC(=O)Nc2cc([C@H]3CC[C@@H](N4CC(C)(C)NC4=O)C3)nn2C(C)(C)C)on1]

Step 4: To a mixture of 1-(cis-3-(3-amino-1-(tert-butyl)-1H-pyrazol-5-yl)cyclopentyl)-4,4-dimethylimidazolidin-2-one (200 mg, 626 μmol), 2-(3-methylisoxazol-5-yl)acetic acid (97.2 mg, 689 μmol) and DIEA (243 mg, 1.88 mmol) in EA (5 mL) was added 2,4,6-tripropyl-1,3,5,2,4,6-trioxatriphosphinane 2,4,6-trioxide (478 mg, 50% Wt in EA, 751 μmol) dropwise at 0° C. under nitrogen atmosphere. The mixture was stirred for 1 hour at 25° C. The reaction was quenched with sat. Na₂CO₃ (10 mL) and extracted with DCM (2*10 mL). The organic layer was washed with more Na₂CO₃ (2*10 mL) and brine (30 mL), and concentrated. The residue was purified by reverse phase flash chromatography with the following conditions: column, C18 silica gel; mobile phase, MeCN in water, 20% to 80% gradient in 20 min; detector, UV 220 nm to N-(1-(tert-butyl)-5-(cis-3-(4,4-dimethyl-2-oxoimidazolidin-1-yl)cyclopentyl)-1H-pyrazol-3-yl)-2-(3-methylisoxazol-5-yl)acetamide (230 mg, 520 μmol, 83.0%) as a white amorphous solid.

m/z (ES⁺) [M+H]⁺=443.20; HPLC tR=0.955 min.

### N-(5-((1S,3R)-3-(4,4-dimethyl-2-oxoimidazolidin-1-yl)cyclopentyl)-1H-pyrazol-3-yl)-2-(3-methylisoxazol-5-yl)acetamide

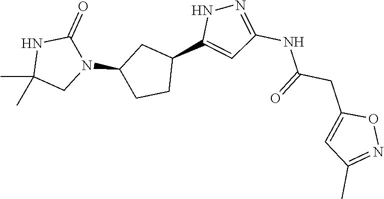 [SMILES: Cc1cc(CC(=O)Nc2cc([C@H]3CC[C@@H](N4CC(C)(C)NC4=O)C3)[nH]n2)on1]

Step 5: A solution of N-(1-(tert-butyl)-5-(cis-3-(4,4-dimethyl-2-oxoimidazolidin-1-yl)cyclopentyl)-1H-pyrazol-3-yl)-2-(3-methylisoxazol-5-yl)acetamide (50 mg, 9.02 μmol) in TFA/H₂O (5 mL, 20:1) was stirred at 90° C. for 0.5 hour. The resulting mixture was concentrated to dryness. This product was combined with from five more identically-prepared batches (each starting with 50 mg), and purified by reverse flash chromatography with the following conditions: column, C18 silica gel; mobile phase, MeCN in water, 0% to 100% gradient in 15 min; detector, UV 220 nm. to afford N-(5-(cis-3-(4,4-dimethyl-2-oxoimidazolidin-1-yl)cyclopentyl)-1H-pyrazol-3-yl)-2-(3-methylisoxazol-5-yl)acetamide (128 mg, 331 μmol, 73.3%) as a white solid.

m/z (ES⁺) [M+H]⁺=387.30; HPLC tR=0.804 min.

### N-(5-((1S,3R)-3-(4,4-dimethyl-2-oxoimidazolidin-1-yl)cyclopentyl)-1H-pyrazol-3-yl)-2-(3-methylisoxazol-5-yl)acetamide

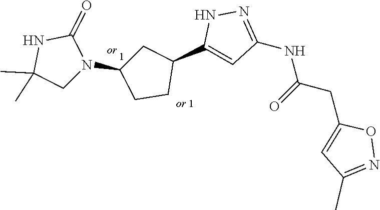 [SMILES: Cc1cc(CC(=O)Nc2cc([C@H]3CC[C@@H](N4CC(C)(C)NC4=O)C3)[nH]n2)on1]

Step 6: Cis-N-(5-(3-(4,4-dimethyl-2-oxoimidazolidin-1-yl)cyclopentyl)-1H-pyrazol-3-yl)-2-(3-methylisoxazol-5-yl)acetamide (128 mg, 331 μmol) was purified by Prep-CHIRAL-HPLC with the following conditions: Column: CHIRALPAK IG, 2*25 cm, 5 m; Mobile Phase A: Hex (0.2% TEA)—HPLC, Mobile Phase B: EtOH: DCM=1:1—HPLC; Flow rate: 20 mL/min; Gradient: 90% B to 90% B in 18 min; Wave Length: 220/254 nm; RT1(min): 5.46; Sample Solvent: EtOH: DCM=1:1—HPLC; Injection Volume: 2.2 mL; Number Of Runs: 2 to afford cis-N-(5-(-3-(4,4-dimethyl-2-oxoimidazolidin-1-yl)cyclopentyl)-1H-pyrazol-3-yl)-2-(3-methylisoxazol-5-yl)acetamide (50.5 mg, 131 μmol, 39.5%) as a white amorphous solid.

m/z (ES⁺) [M+H]⁺=387.15; HPLC tR=0.653 min.

¹H NMR (400 MHz, DMSO) δ 1.17 (d, J=2.7 Hz, 6H), 1.63 (dt, J=18.7, 11.4, 11.4 Hz, 3H), 1.76 (d, J=9.9 Hz, 1H), 1.91-2.13 (m, 2H), 2.20 (d, J=1.4 Hz, 3H), 3.05 (d, J=9.6 Hz, 3H), 3.83 (s, 2H), 4.22 (q, J=8.3, 8.3, 8.3 Hz, 1H), 5.97-6.51 (m, 3H), 10.65 (s, 1H), 12.14 (s, 1H).

### N-(5-((1R,3S)-3-(4,4-dimethyl-2-oxoimidazolidin-1-yl)cyclopentyl)-1H-pyrazol-3-yl)-2-(3-methylisoxazol-5-yl)acetamide

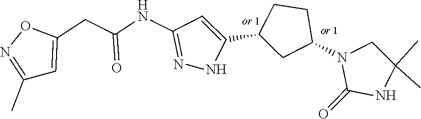 [SMILES: Cc1cc(CC(=O)Nc2cc([C@@H]3CC[C@H](N4CC(C)(C)NC4=O)C3)[nH]n2)on1]

Step 7: Cis-N-(5-(3-(4,4-dimethyl-2-oxoimidazolidin-1-yl)cyclopentyl)-1H-pyrazol-3-yl)-2-(3-methylisoxazol-5-yl)acetamide (128 mg, 331 μmol) was purified by Prep-CHIRAL-HPLC with the following conditions: Column: CHIRALPAK IG, 2*25 cm, 5 m; Mobile Phase A: Hex (0.2% TEA)—HPLC, Mobile Phase B: EtOH: DCM=1:1—HPLC; Flow rate: 20 mL/min; Gradient: 90% B to 90% B in 18 min; Wave Length: 220/254 nm; RT2(min): 8.40; Sample Solvent: EtOH: DCM=1:1—HPLC; Injection Volume: 2.2 mL; Number Of Runs: 2 to afford N-(5-((1R,3S)-3-(4,4-dimethyl-2-oxoimidazolidin-1-yl)cyclopentyl)-1H-pyrazol-3-yl)-2-(3-methylisoxazol-5-yl)acetamide (48.2 mg, 125 μmol, 37.7%) as a white amorphous solid.

m/z (ES⁺) [M+H]⁺=387.15; HPLC tR=0.662 min.

¹H NMR (400 MHz, DMSO-d₆) δ 1.17 (d, J=2.7 Hz, 6H), 1.41-1.68 (m, 3H), 1.76 (d, J=9.7 Hz, 1H), 2.02 (dd, J=18.5, 7.6 Hz, 2H), 2.20 (s, 3H), 3.05 (d, J=8.9 Hz, 3H), 3.83 (s, 2H), 4.22 (q, J=8.4, 8.3, 8.3 Hz, 1H), 6.11-6.58 (m, 3H), 10.65 (s, 1H), 12.14 (s, 1H).

### Example 10

### 2-(3-methylisoxazol-5-yl)-N-(5-((1S,3R)-3-((4-(prop-1-en-2-yl)pyridin-3-yl)oxy)cyclopentyl)-1H-pyrazol-3-yl)acetamide and 2-(3-methylisoxazol-5-yl)-N-(5-((1R,3S)-3-((4-(prop-1-en-2-yl)pyridin-3-yl)oxy)cyclopentyl)-1H-pyrazol-3-yl)acetamide

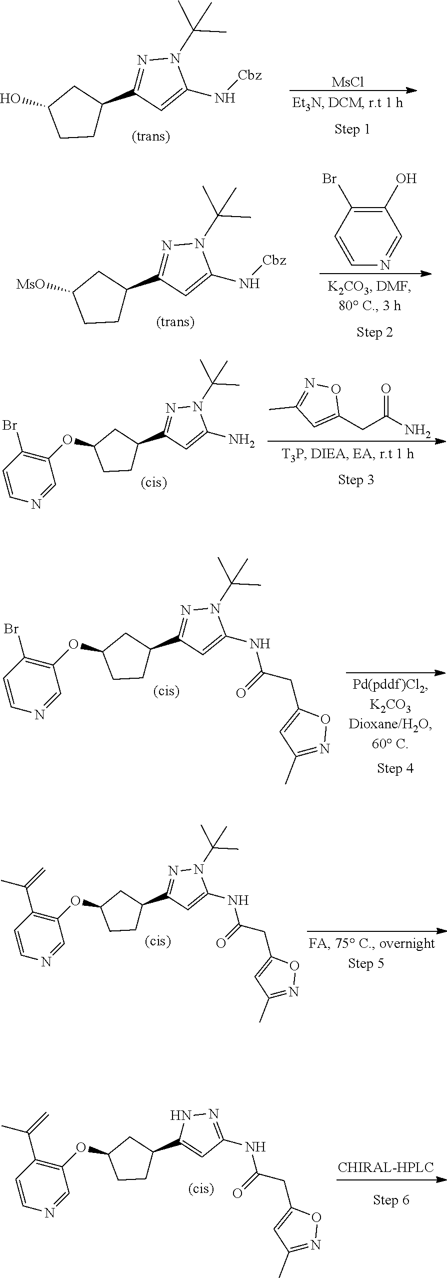

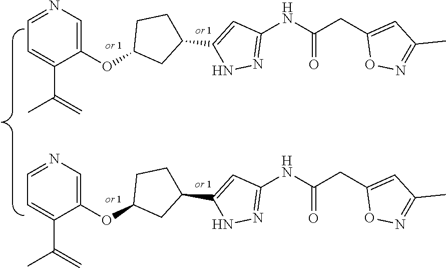 [SMILES: C=C(C)c1ccncc1O[C@@H]1CC[C@H](c2cc(NC(=O)Cc3cc(C)no3)n[nH]2)C1 ; C=C(C)c1ccncc1O[C@H]1CC[C@@H](c2cc(NC(=O)Cc3cc(C)no3)n[nH]2)C1]

### (1S,3S)-3-(5-(((benzyloxy)carbonyl)amino)-1-(tert-butyl)-1H-pyrazol-3-yl)cyclopentyl methanesulfonate

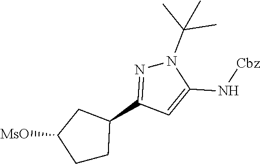 [SMILES: CC(C)(C)n1nc([C@H]2CC[C@H](OS(C)(=O)=O)C2)cc1NC(=O)OCc1ccccc1]

Step 1: To a mixture of benzyl (1-(tert-butyl)-5-(trans-3-hydroxycyclopentyl)-1H-pyrazol-3-yl)carbamate (250 mg, 699 μmol) and Et₃N (84.9 mg, 839 μmol) in DCM (10 mL) was added MsCl (303 mg, 2.10 mmol) dropwise at 0° C. under nitrogen atmosphere. The mixture was stirred for 1 h at 25° C. The reaction mixture was diluted with H₂O (50 mL), and the aqueous phase was extracted with EA (100 mL) three times. The combined organic layers were washed with brine, dried over sodium sulfate, filtered, and concentrated in vacuo, resulting in trans-3-(3-(((benzyloxy)carbonyl)amino)-1-(tert-butyl)-1H-pyrazol-5-yl)cyclopentyl methanesulfonate (300 mg, 98.5%) as a yellow oil.

m/z (ES⁺) [M+H]⁺=436.25; HPLC tR=1.202 min.

### 3-((1S,3R)-3-((4-bromopyridin-3-yl)oxy)cyclopentyl)-1-(tert-butyl)-1H-pyrazol-5-amine

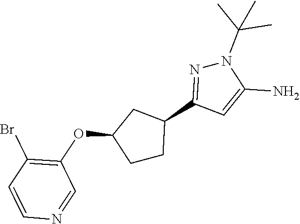 [SMILES: CC(C)(C)n1nc([C@H]2CC[C@@H](Oc3cnccc3Br)C2)cc1N]

Step 2: A resealable reaction vial was charged with trans-3-(3-(((benzyloxy)carbonyl)amino)-1-(tert-butyl)-1H-pyrazol-5-yl)cyclopentyl methanesulfonate (300 mg, 689 μmol), 4-bromopyridin-3-ol (144 mg, 827 μmol), K₂CO₃ (286 mg, 2.07 mmol), DMF (10 mL), and a stirbar before being evacuated and purged with nitrogen three times, and the mixture was stirred for 3 h at 80° C. The reaction mixture was diluted with H₂O (50 mL), and the aqueous phase was extracted with EA (100 mL) three times. The combined organic layers were washed with brine, dried over sodium sulfate, filtered, and concentrated in vacuo. The residue was purified by prep-TLC (PE/EA; ratio:4/1) to afford 5-(cis-3-((4-bromopyridin-3-yl)oxy)cyclopentyl)-1-(tert-butyl)-1H-pyrazol-3-amine (120 mg, 45.5%) as a yellow oil.

m/z (ES⁺) [M+H]⁺=379.20; HPLC tR=0.810 min.

### N-(3-((1S,3R)-3-((4-bromopyridin-3-yl)oxy)cyclopentyl)-1-(tert-butyl)-1H-pyrazol-5-yl)-2-(3-methylisoxazol-5-yl)acetamide

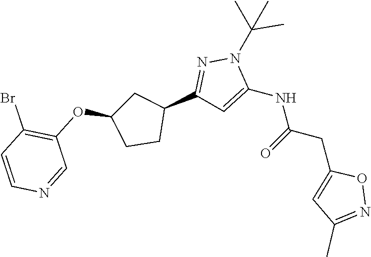 [SMILES: Cc1cc(CC(=O)Nc2cc([C@H]3CC[C@@H](Oc4cnccc4Br)C3)nn2C(C)(C)C)on1]

Step 3: A resealable reaction vial was charged with 5-(cis-3-((4-bromopyridin-3-yl)oxy)cyclopentyl)-1-(tert-butyl)-1H-pyrazol-3-amine (256 mg, 675 μmol), DIEA (349 mg, 2.70 mmol), 2-(3-methylisoxazol-5-yl)acetamide (114 mg, 810 μmol) in EA (5 mL) was added T3P (644 mg, 2.02 mmol) drop wise at 0° C. under nitrogen atmosphere. The mixture was stirred for 1 h at 25° C. The reaction mixture was diluted with H₂O (50 mL), and the aqueous phase was extracted with EA (50 mL) three times. The combined organic layers were washed with brine, dried over sodium sulfate, filtered, and concentrated in vacuo. The residue was purified by prep-TLC (DCM/MeOH; ratio:20/1) to afford N-(5-(cis-3-((4-bromopyridin-3-yl)oxy)cyclopentyl)-1-(tert-butyl)-1H-pyrazol-3-yl)-2-(3-methylisoxazol-5-yl)acetamide (240 mg, 70.8%) as a yellow oil.

m/z (ES⁺) [M+H]=502.10; HPLC tR=0.747 min.

### N-(1-(tert-butyl)-3-((1S,3R)-3-((4-(prop-1-en-2-yl)pyridin-3-yl)oxy)cyclopentyl)-1H-pyrazol-5-yl)-2-(3-methylisoxazol-5-yl)acetamide

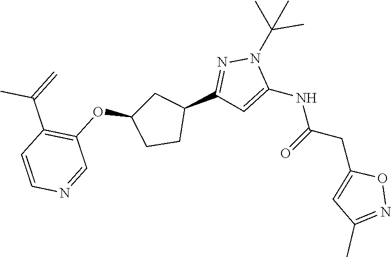 [SMILES: C=C(C)c1ccncc1O[C@@H]1CC[C@H](c2cc(NC(=O)Cc3cc(C)no3)n(C(C)(C)C)n2)C1]

Step 4: A resealable reaction vial was charged with N-(5-((1S,3R)-3-((4-bromopyridin-3-yl)oxy)cyclopentyl)-1-(tert-butyl)-1H-pyrazol-3-yl)-2-(3-methylisoxazol-5-yl)acetamide (60 mg, 0.12 mmol), prop-1-en-2-ylboronic acid (12 mg, 0.14 mmol), Pd(dppf)Cl2 (8.7 mg, 12 μmol), K₂CO₃ (46 mg, 0.36 mmol), dioxane/H₂O (1 mL) was added, and a stirbar before being evacuated and purged with nitrogen three times, and the mixture was stirred for 1 h at 60° C. The reaction mixture was diluted with H₂O (50 mL), and the aqueous phase was extracted with EA (100 mL) three times. The combined organic layers were washed with brine, dried over sodium sulfate, filtered, and concentrated in vacuo. The residue was purified by prep-TLC (DCM/MeOH; ratio:20/1) to afford N-(1-(tert-butyl)-5-((1S,3R)-3-((4-(prop-1-en-2-yl)pyridin-3-yl)oxy)cyclopentyl)-1H-pyrazol-3-yl)-2-(3-methylisoxazol-5-yl)acetamide (40 mg, 72%) as an orange oil.

m/z (ES⁺) [M+H]=464.25; HPLC tR=1.100 min.

### 2-(3-methylisoxazol-5-yl)-N-(5-((1S,3R)-3-((4-(prop-1-en-2-yl)pyridin-3-yl)oxy)cyclopentyl)-1H-pyrazol-3-yl)acetamide

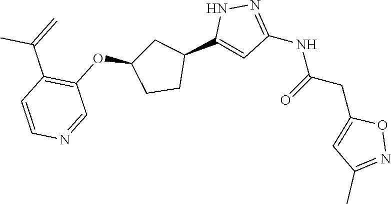 [SMILES: C=C(C)c1ccncc1O[C@@H]1CC[C@H](c2cc(NC(=O)Cc3cc(C)no3)n[nH]2)C1]

Step 5: A resealable reaction vial was charged with N-(1-(tert-butyl)-5-(cis-3-((4-(prop-1-en-2-yl)pyridin-3-yl)oxy)cyclopentyl)-1H-pyrazol-3-yl)-2-(3-methylisoxazol-5-yl)acetamide (200 mg, 431 μmol), FA (5 mL) and a stirbar before being evacuated and purged with nitrogen three times, and the mixture was stirred for 12 h at 75° C. The resulting crude material was purified by C18 flash (acetonitrile/water/0.1% formic acid). Concentration in vacuo afforded 2-(3-methylisoxazol-5-yl)-N-(5-(cis-3-((4-(prop-1-en-2-yl)pyridin-3-yl)oxy)cyclopentyl)-1H-pyrazol-3-yl)acetamide (70 mg, 39.8%) as a colorless oil.

m/z (ES⁺) [M+H]⁺=408.30; HPLC tR=0.790 min.

### 2-(3-methylisoxazol-5-yl)-N-(5-((1S,3R)-3-((4-(prop-1-en-2-yl)pyridin-3-yl)oxy)cyclopentyl)-1H-pyrazol-3-yl)acetamide

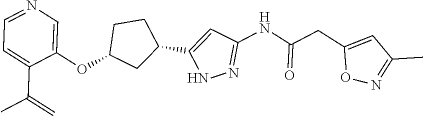 [SMILES: C=C(C)c1ccncc1O[C@@H]1CC[C@H](c2cc(NC(=O)Cc3cc(C)no3)n[nH]2)C1]

2-(3-methylisoxazol-5-yl)-N-(5-(cis-3-((4-(prop-1-en-2-yl)pyridin-3-yl)oxy)cyclopentyl)-1H-pyrazol-3-yl)acetamide (70 mg, 0.17 mmol) was purified by chiral pre-HPLC (Column: CHIRALPAK IG, 2*25 cm, 5 m; Mobile Phase A: Hex(0.2% DEA)—HPLC, Mobile Phase B: EtOH: DCM=1:1—HPLC; Flow rate: 20 mL/min; Gradient: 50% B to 50% B in 32 min; Wave Length: 220/254 nm; RT1(min): 15.69; RT2(min): 25.34; Sample Solvent: EtOH: DCM=1:1—HPLC; Injection Volume: 0.8 mL; Number Of Runs: 4). Lyophilization afforded 2-(3-methylisoxazol-5-yl)-N-(5-((1S,3R)-3-((4-(prop-1-en-2-yl)pyridin-3-yl)oxy)cyclopentyl)-1H-pyrazol-3-yl)acetamide (28.9 mg, 41%) as a white solid.

m/z (ES⁺) [M+H]⁺=408.35; HPLC tR=0.962 min.

¹H NMR (400 MHz, DMSO-d₆) δ 12.16 (s, 1H), 10.62 (s, 1H), 8.31 (s, 1H), 8.15 (d, J=4.8 Hz, 1H), 7.17 (d, J=4.8 Hz, 1H), 6.29 (s, 1H), 6.22 (s, 1H), 5.27-5.20 (m, 2H), 5.05 (s, 1H), 3.83 (s, 2H), 3.18-3.07 (m, 1H), 2.66 (dt, J=14.4, 7.3 Hz, 1H), 2.20 (s, 3H), 2.10-1.97 (m, 5H), 1.89 (d, J=13.4 Hz, 1H), 1.76 (td, J=14.0, 12.3, 5.3 Hz, 2H).

### 2-(3-methylisoxazol-5-yl)-N-(5-((1R,3S)-3-((4-(prop-1-en-2-yl)pyridin-3-yl)oxy)cyclopentyl)-1H-pyrazol-3-yl)acetamide

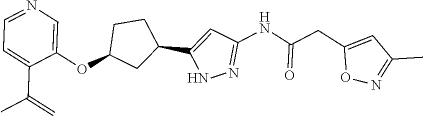 [SMILES: C=C(C)c1ccncc1O[C@H]1CC[C@@H](c2cc(NC(=O)Cc3cc(C)no3)n[nH]2)C1]

2-(3-methylisoxazol-5-yl)-N-(5-(cis-3-((4-(prop-1-en-2-yl)pyridin-3-yl)oxy)cyclopentyl)-1H-pyrazol-3-yl)acetamide (70 mg, 0.17 mmol) was purified by chiral Pre-HPLC (Column: CHIRALPAK IG, 2*25 cm, 5 m; Mobile Phase A: Hex(0.2% DEA)—HPLC, Mobile Phase B: EtOH: DCM=1:1—HPLC; Flow rate: 20 mL/min; Gradient: 50% B to 50% B in 32 min; Wave Length: 220/254 nm; RT1(min): 15.69; RT2(min): 25.34; Sample Solvent: EtOH: DCM=1:1—HPLC; Injection Volume: 0.8 mL; Number Of Runs: 4). Lyophilization2-(3-methylisoxazol-5-yl)-N-(5-((1R,3S)-3-((4-(prop-1-en-2-yl)pyridin-3-yl)oxy)cyclopentyl)-1H-pyrazol-3-yl)acetamide (25.9 mg, 37%) as a white solid.

m/z (ES⁺) [M+H]⁺=408.35; HPLC tR=0.928 min.

¹H NMR (400 MHz, DMSO-d₆) δ 12.16 (s, 1H), 10.63 (s, 1H), 8.31 (s, 1H), 8.15 (d, J=4.8 Hz, 1H), 7.17 (d, J=4.8 Hz, 1H), 6.29 (s, 1H), 6.22 (s, 1H), 5.23 (qd, J=2.7, 2.0, 1.3 Hz, 2H), 5.06 (d, J=4.4 Hz, 1H), 3.83 (s, 2H), 3.13 (p, J=9.2 Hz, 1H), 2.67 (tt, J=14.3, 7.3 Hz, 1H), 2.20 (s, 3H), 2.10-1.97 (m, 5H), 1.89 (d, J=13.0 Hz, 1H), 1.85-1.69 (m, 2H).

Additional compounds prepared according to the methods of Example 9 or Example 10 are depicted in Table 6 below.

TABLE 6
Additional Exemplary Compounds

| | | | MS |
|---|---|---|---|
| Compound | Structure | Proton NMR | [M + 1] |
| N-(5-((1S,3R)-3-((4- cyclopropylisothiazol-3- yl)oxy)cyclopentyl)-1H- pyrazol-3-yl)-2-(3- methylisoxazol-5- yl)acetamide | 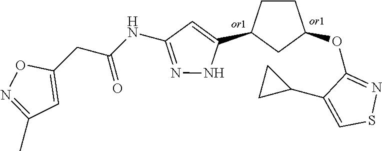 [SMILES: Cc1cc(CC(=O)Nc2cc([C@H]3CC[C@@H](Oc4nscc4C4CC4)C3)[nH]n2)on1] | ¹H NMR (400 MHz, DMSO-d₆) 12.14 (s, 1H), 10.62 (s, 1H), 8.26 (d, J = 0.6 Hz, 1H), 6.34 (s, 1H), 6.22 (s, 1H), 5.34 (q, J = 4.8, 3.9 Hz, 1H), 3.82 (s, 2H), 3.22- 3.02 (m, 1H), 2.67- 2.55 (m, 1H), 2.20 (s, 3H), 2.12-1.97 (m, 2H), 1.92 (d, J = 13.2 Hz, 1H), 1.86-1.65 (m, 3H), 0.89-0.77 (m, 2H), 0.69-0.52 (m, 2H). | 414.10 |
| N-(5-((1R,3S)-3-((4- cyclopropylisothiazol-3- yl)oxy)cyclopentyl)-1H- pyrazol-3-yl)-2-(3- methylisoxazol-5- yl)acetamide | 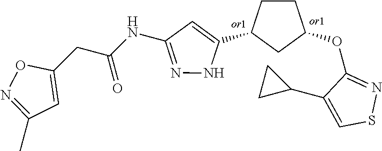 [SMILES: Cc1cc(CC(=O)Nc2cc([C@@H]3CC[C@H](Oc4nscc4C4CC4)C3)[nH]n2)on1] | ¹H NMR (400 MHz, DMSO-d₆) 12.14 (s, 1H), 10.62 (s, 1H), 8.26 (s, 1H), 6.34 (s, 1H), 6.22 (s, 1H), 5.34 (q, J = 5.0, 3.5 Hz, 1H), 3.82 (s, 2H), 3.15 (p, J = 8.6 Hz, 1H), 2.60 (dt, J = 14.5, 7.5 Hz, 1H), 2.20 (s, 3H), 2.12-1.97 (m, 2H), 1.92 (d, J = 13.2 Hz, 1H), 1.83-1.66 (m, 3H), 0.90-0.75 (m, 2H), 0.72-0.53 (m, 2H). | 414.10 |
| 2-(3-methylisoxazol-5-yl)-N- (5-((1S,3R)-3-(5-oxo-4,6- diazaspiro[2.4]heptan-6- yl)cyclopentyl)-1H-pyrazol-3- yl)acetamide | 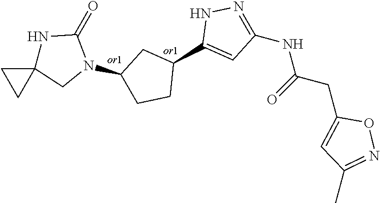 [SMILES: Cc1cc(CC(=O)Nc2cc([C@H]3CC[C@@H](N4CC5(CC5)NC4=O)C3)[nH]n2)on1] | ¹H NMR (400 MHz, DMSO-d₆) 12.14 (s, 1H), 10.64 (s, 1H), 6.44 (s, 1H), 6.30 (s, 1H), 6.22 (s, 1H), 4.28 (dq, J = 9.8, 7.4 Hz, 1H), 3.83 (s, 2H), 3.36 (s, 2H), 3.05 (dq, J = 11.3, 8.2 Hz, 1H), 2.20 (s, 3H), 2.13-1.95 (m, 2H), 1.80 (dq, J = 11.1, 7.2, 6.5 Hz, 1H), 1.74- 1.54 (m, 3H), 0.69 (td, J = 6.8, 6.1, 2.4 Hz, 2H), 0.62 (td, J = 6.5, 6.0, 2.3 Hz, 2H). | 385.15 |
| 2-(3-methylisoxazol-5-yl)-N- (5-((1R,3S)-3-(5-oxo-4,6- diazaspiro[2.4]heptan-6- yl)cyclopentyl)-1H-pyrazol-3- yl)acetamide | 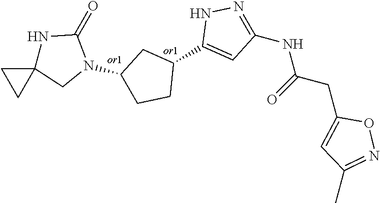 [SMILES: Cc1cc(CC(=O)Nc2cc([C@@H]3CC[C@H](N4CC5(CC5)NC4=O)C3)[nH]n2)on1] | ¹H NMR (400 MHz, DMSO-d₆) 12.14 (s, 1H), 10.64 (s, 1H), 6.44 (s, 1H), 6.30 (s, 1H), 6.22 (s, 1H), 4.28 (p, J = 8.3 Hz, 1H), 3.83 (s, 2H), 3.36 (s, 2H), 3.05 (p, J = 8.2 Hz, 1H), 2.20 (s, 3H), 2.14-1.94 (m, 2H), 1.81 (dt, J = 13.1, 8.1 Hz, 1H), 1.74-1.52 (m, 3H), 0.72-0.65 (m, 2H), 0.65-0.57 (m, 2H). | 385.10 |
| 2-(3-methylisoxazol-5-yl)-N- (5-((1R,3S)-3-(pyridin-3- yloxy)cyclopentyl)-1H- pyrazol-3-yl)acetamide | 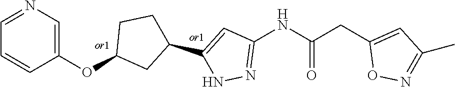 [SMILES: Cc1cc(CC(=O)Nc2cc([C@@H]3CC[C@H](Oc4cccnc4)C3)[nH]n2)on1] | ¹H NMR (400 MHz, Chloroform-d) 7.94 (d, J = 2.4 Hz, 2H), 7.42 (s, 1H), 6.83 (s, 1H), 6.60 (s, 1H), 5.24 (s, 1H), 4.68 (s, 1H), 3.83 (s, 1H), 3.25 (p, J = 8.2 Hz, 1H), 2.55 (dt, J = 15.1, 8.0 Hz, 1H), 2.18 (d, J = 8.0 Hz, 1H), 2.00-1.91 (m, 5H), 1.16 (t, J = 6.6 Hz, 6H). | 368.15 |
| 2-(3-methylisoxazol-5-yl)-N- (5-((1S,3R)-3-(pyridin-3- yloxy)cyclopentyl)-1H- pyrazol-3-yl)acetamide | 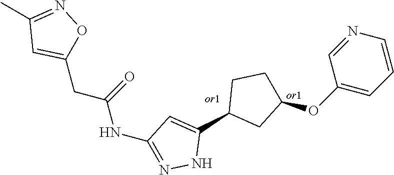 [SMILES: Cc1cc(CC(=O)Nc2cc([C@H]3CC[C@@H](Oc4cccnc4)C3)[nH]n2)on1] | ¹H NMR (400 MHz, Chloroform-d) 7.96 (d, J = 2.4 Hz, 1H), 7.93 (s, 1H), 7.40 (s, 1H), 6.95 (s, 1H), 6.62 (s, 1H), 5.24 (s, 1H), 4.71 (s, 1H), 3.83 (s, 1H), 3.25 (p, J = 8.1 Hz, 1H), 2.55 (dd, J = 14.7, 7.6 Hz, 1H), 2.18 (d, J = 7.1 Hz, 1H), 1.95 (d, J = 18.1 Hz, 5H), 1.18 (dd, J = 6.7, 4.7 Hz, 6H). | 368.20 |
| N-(5-((1S,3R)-3-(4-methyl-2- oxo-2,3-dihydro-1H-imidazol- 1-yl)cyclopentyl)-1H-pyrazol- 3-yl)-2-(3-methylisoxazol-5- yl)acetamide | 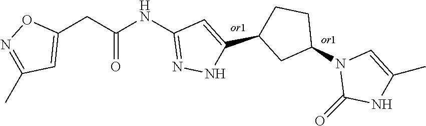 [SMILES: Cc1cc(CC(=O)Nc2cc([C@H]3CC[C@@H](n4cc(C)[nH]c4=O)C3)[nH]n2)on1] | ¹H NMR (400 MHz, DMSO-d₆) 12.18 (s, 1H), 10.65 (s, 1H), 9.85 (s, 1H), 6.34 (s, 1H), 6.21 (d, J = 10.5 Hz, 2H), 4.47- 4.35 (m, 1H), 3.83 (s, 2H), 3.16-3.05 (m, 1H), 2.26 (dd, J = 12.7, 6.6 Hz, 1H), 2.20 (s, 3H), 2.01 (s, 2H), 1.89 (d, J = 1.3 Hz, 3H), 1.85-1.64 (m, 3H). | 371.05 |
| N-(5-((1R,3S)-3-(4-methyl-2- ox0-2,3-dihydro-1H-imidazol- 1-yl)cyclopentyl)-1H-pyrazol- 3-yl)-2-(3-methylisoxazol-5- yl)acetamide | 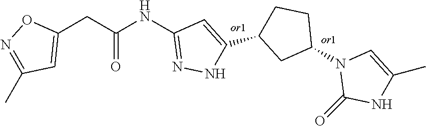 [SMILES: Cc1cc(CC(=O)Nc2cc([C@@H]3CC[C@H](n4cc(C)[nH]c4=O)C3)[nH]n2)on1] | ¹H NMR (400 MHz, DMSO-d₆) 12.1 (s, 1H), 10.65 (s, 1H), 9.85 (s, 1H), 6.34 (s, 1H), 6.21 (d, J = 10.5 Hz, 2H), 4.47- 4.35 (m, 1H), 3.83 (s, 2H), 3.16-3.05 (m, 1H), 2.26 (dd, J = 12.7, 6.6 Hz, 1H), 2.20 (s, 3H), 2.01 (s, 2H), 1.89 (d, J = 1.3 Hz, 3H), 1.85-1.64 (m, 3H). | 371.10 |
| 2-(3-methylisoxazol-5-yl)-N- (5-((1R,3S)-3-((5- methylpyridin-3- yl)oxy)cyclopentyl)-1H- pyrazol-3-yl)acetamide | 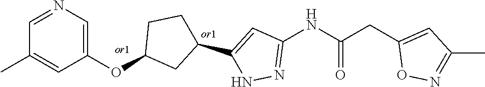 | ¹H NMR (400 MHz, Chloroform-d) 8.75 (s, 1H), 8.14 (d, J = 2.7 Hz, 1H), 8.12- 8.06 (m, 1H), 7.05 (t, J = 2.3 Hz, 1H), 6.54 (s, 1H), 6.17 (s, 1H), 4.93 (dt, J = 5.7, 2.9 Hz, 1H), 3.88 (s, 2H), 3.33 (p, J = 7.9 Hz, 1H), 2.64- 2.47 (m, 1H), 2.33 (d, J = 11.5 Hz, 6H), 2.25-2.00 (m, 2H), 2.03-1.89 (m, 3H). | 382.15 |
| 2-(3-methylisoxazol-5-yl)-N- (5-((1S,3R)-3-((5- methylpyridin-3- yl)oxy)cyclopentyl)-1H- pyrazol-3-yl)acetamide | 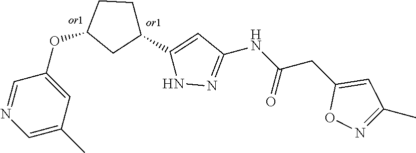 [SMILES: Cc1cncc(O[C@@H]2CC[C@H](c3cc(NC(=O)Cc4cc(C)no4)n[nH]3)C2)c1] | ¹H NMR (400 MHz, Chloroform-d) 8.88 (s, 1H), 8.14 (d, J = 2.7 Hz, 1H), 8.08 (d, J = 1.6 Hz, 1H), 7.06 (d, J = 2.5 Hz, 1H), 6.53 (s, 1H), 6.17 (s, 1H), 4.92 (q, J = 2.8 Hz, 1H), 3.88 (s, 2H), 3.32 (p, J = 7.8 Hz, 1H), 2.57 (ddd, J = 15.0, 9.4, 6.1 Hz, 1H), 2.32 (d, J = 13.3 Hz, 6H), 2.27-1.89 (m, 5H). | 382.15 |
| 2-(3-methylisoxazol-5-yl)-N- (5-((1R,3S)-3-((6- methylpyridin-3- yl)oxy)cyclopentyl)-1H- pyrazol-3-yl)acetamide | 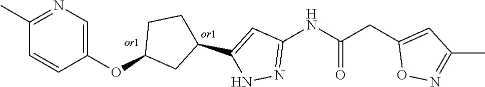 | ¹H NMR (400 MHz, DMSO-d₆) 12.16 (s, 1H), 10.64 (s, 1H), 8.10 (d, J = 2.9 Hz, 1H), 7.25 (dd, J = 8.5, 3.0 Hz, 1H), 7.15 (d, J = 8.5 Hz, 1H), 6.31 (s, 1H), 6.23 (s, 1H), 4.92 (s, 1H), 3.83 (s, 2H), 3.17-3.08 (m, 1H), 2.60 (dd, J = 13.9, 7.0 Hz, 1H), 2.38 (s, 3H), 2.20 (s, 3H), 2.03 (s, 2H), 1.87 (s, 1H), 1.79 (dd, J = 23.7, 13.8 Hz, 1H), 1.71 (s, 1H). | 382.20 |
| 2-(3-methylisoxazol-5-yl)-N- (5-((1S,3R)-3-((6- methylpyridin-3- yl)oxy)cyclopentyl)-1H- pyrazol-3-yl)acetamide | 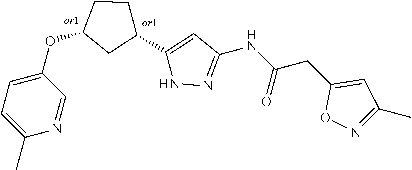 [SMILES: Cc1ccc(O[C@@H]2CC[C@H](c3cc(NC(=O)Cc4cc(C)no4)n[nH]3)C2)cn1] | ¹H NMR (400 MHz, DMSO-d₆) 12.16 (s, 1H), 10.64 (s, 1H), 8.10 (d, J = 2.9 Hz, 1H), 7.25 (dd, J = 8.5, 3.0 Hz, 1H), 7.15 (d, J = 8.5 Hz, 1H), 6.31 (s, 1H), 6.23 (s, 1H), 4.95- 4.89 (m, 1H), 3.83 (s, 2H), 3.12 (p, J = 8.9 Hz, 1H), 2.59 (dt, J = 14.3, 7.2 Hz, 1H), 2.38 (s, 3H), 2.20 (s, 3H), 2.10-1.94 (m, 2H), 1.91-1.85 (m, 1H), 1.84-1.64 (m, 2H). | 382.15 |
| 2-(3-methylisoxazol-5-yl)-N- (5-((1R,3S)-3-((2- methylpyridin-3- yl)oxy)cyclopentyl)-1H- pyrazol-3-yl)acetamide | 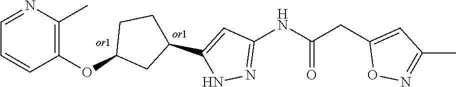 [SMILES: Cc1cc(CC(=O)Nc2cc([C@@H]3CC[C@H](Oc4cccnc4C)C3)[nH]n2)on1] | ¹H NMR (400 MHz, Chloroform-d) 8.72 (s, 1H), 8.10 (t, J = 3.1 Hz, 1H), 7.13 (d, J = 3.1 Hz, 2H), 6.51 (s, 1H), 6.17 (s, 1H), 4.89 (q, J = 4.0, 2.9 Hz, 1H), 3.87 (s, 2H), 3.33 (p, J = 7.8 Hz, 1H), 2.60 (ddd, J = 14.9, 9.2, 6.2 Hz, 1H), 2.49 (s, 3H), 2.31 (s, 3H), 2.27-2.16 (m, 1H), 2.14-1.96 (m, 4H). | 382.20 |
| 2-(3-methylisoxazol-5-yl)-N- (5-((1S,3R)-3-((2- methylpyridin-3- yl)oxy)cyclopentyl)-1H- pyrazol-3-yl)acetamide | 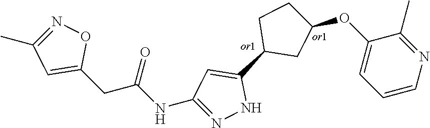 [SMILES: Cc1cc(CC(=O)Nc2cc([C@H]3CC[C@@H](Oc4cccnc4C)C3)[nH]n2)on1] | ¹H NMR (400 MHz, Chloroform-d) 9.02 (s, 1H), 8.09 (dd, J = 3.7, 2.5 Hz, 1H), 7.15-7.07 (m, 2H), 6.52 (s, 1H), 6.16 (s, 1H), 4.88 (td, J = 5.5, 2.7 Hz, 1H), 3.87 (s, 2H), 3.32 (p, J = 7.8 Hz, 1H), 2.60 (ddd, J = 14.9, 9.1, 6.2 Hz, 1H), 2.47 (s, 3H), 2.32 (s, 3H), 2.25-2.13 (m, 1H), 2.13-1.89 (m, 4H). | 382.20 |
| 2-(3-methylisoxazol-5-yl)-N- (5-((1R,3S)-3- phenoxycyclopentyl)-1H- pyrazol-3-yl)acetamide | 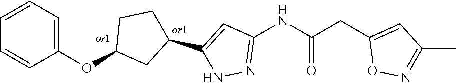 [SMILES: Cc1cc(CC(=O)Nc2cc([C@@H]3CC[C@H](Oc4ccccc4)C3)[nH]n2)on1] | ¹H NMR (400 MHz, DMSO-d₆) 12.15 (s, 1H), 10.63 (s, 1H), 7.32-7.22 (m, 2H), 6.95-6.86 (m, 3H), 6.32 (s, 1H), 6.22 (s, 1H), 4.89 (d, J = 5.2 Hz, 1H), 3.83 (s, 2H), 3.16-3.05 (m, 1H), 2.60 (dt, J = 14.2, 7.4 Hz, 1H), 2.20 (s, 3H), 2.11-1.95 (m, 2H), 1.92-1.82 (m, 1H), 1.81-1.66 (m, 2H). | 367.30 |
| 2-(3-methylisoxazol-5-yl)-N- (5-((1S,3R)-3- phenoxycyclopentyl)-1H- pyrazol-3-yl)acetamide | 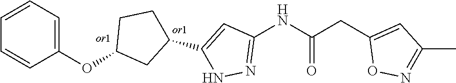 [SMILES: Cc1cc(CC(=O)Nc2cc([C@H]3CC[C@@H](Oc4ccccc4)C3)[nH]n2)on1] | ¹H NMR (400 MHz, DMSO-d₆) 12.15 (s, 1H), 10.63 (s, 1H), 7.31-7.22 (m, 2H), 6.90 (ddd, J = 6.7, 3.3, 1.9 Hz, 3H), 6.32 (d, J = 2.2 Hz, 1H), 6.22 (s, 1H), 4.89 (d, J = 4.9 Hz, 1H), 3.83 (s, 2H), 3.11 (q, J = 8.8 Hz, 1H), 2.60 (dt, J = 14.2, 7.3 Hz, 2H), 2.20 (s, 3H), 2.10-1.94 (m, 2H), 1.89-1.65 (m, 3H). | 367.30 |
| rel-3-(methoxymethyl)-1- methyl-N-(3-((1R,3S)-3-((4- methylpyridin-3- yl)oxy)cyclopentyl)-1H- pyrazol-5-yl)-1H-pyrazole-5- carboxamide | 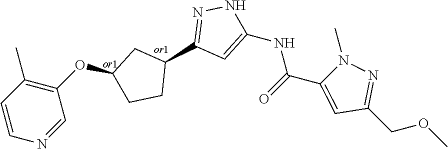 [SMILES: COCc1cc(C(=O)Nc2cc([C@H]3CC[C@@H](Oc4cnccc4C)C3)n[nH]2)n(C)n1] | ¹H NMR (400 MHz, CDCl₃) 1.82-2.21 (m, 5H), 2.23 (s, 3H), 2.56-2.79 (m, 1H), 3.19 (p, J = 8.3 Hz, 1H), 3.51 (s, 3H), 4.16 (s, 3H), 4.53 (s, 2H), 4.96 (q, J = 5.6, 4.9 Hz, 1H), 6.76 (s, 1H), 6.90 (s, 1H), 7.09 (d, J = 4.7 Hz, 1H), 8.07-8.22 (m, 2H), 10.51 (s, 1H) | 411.20 |
| rel-3-(methoxymethyl)-1- methyl-N-(3-((1R,3S)-3-((4- methylpyridin-3- yl)oxy)cyclopentyl)-1H- pyrazol-5-yl)-1H-pyrazole-5- carboxamide | 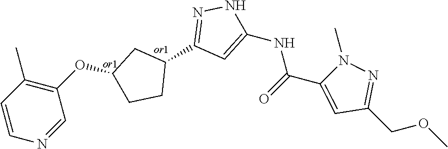 [SMILES: COCc1cc(C(=O)Nc2cc([C@@H]3CC[C@H](Oc4cnccc4C)C3)n[nH]2)n(C)n1] | ¹H NMR (400 MHz, CDCl₃) 2.07 (ddtd, J = 31.4, 17.4, 11.2, 10.0, 5.6 Hz, 5H), 2.22 (s, 3H), 2.65 (dt, J = 14.7, 7.4 Hz, 1H), 3.18 (q, J = 8.3 Hz, 1H), 3.50 (s, 3H), 4.15 (s, 3H), 4.52 (s, 2H), 4.94 (dd, J = 8.6, 4.6 Hz, 1H), 6.75 (s, 1H), 6.90 (s, 1H), 7.08 (d, J = 4.7 Hz, 1H), 7.98- 8.22 (m, 2H), 10.66 (s, 1H) | 411.20 |
| rel-2-(3-methylisoxazol-5-yl)- N-(3-((1R,3S)-3-(pyrimidin-5- yloxy)cyclopentyl)-1H- pyrazol-5-yl)acetamide | 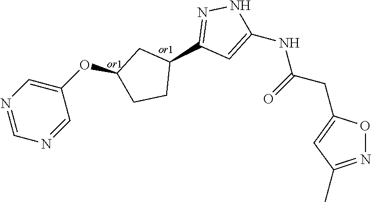 [SMILES: Cc1cc(CC(=O)Nc2cc([C@H]3CC[C@@H](Oc4cncnc4)C3)n[nH]2)on1] | ¹H NMR (400 MHz, DMSO-d₆) 1.77 (ddq, J = 18.0, 9.1, 5.2, 4.3 Hz, 2H), 1.85-1.96 (m, 1H), 2.07 (dh, J = 13.7, 7.0 Hz, 2H), 2.20 (s, 3H), 2.65 (dt, J = 14.3, 7.7 Hz, 1H), 3.15 (p, J = 9.0 Hz, 1H), 3.83 (s, 1H), 5.08 (dq, J = 10.0, 5.8, 4.5 Hz, 1H), 6.27 (d, J = 39.8 Hz, 2H), 8.53 (s, 2H), 8.79 (s, 1H), 10.64 (s, 1H), 12.17 (s, 1H) | 369.25 |
| rel-2-(3-methylisoxazol-5-yl)- N-(3-((1R,3S)-3-(pyrimidin-5- yloxy)cyclopentyl)-1H- pyrazol-5-yl)acetamide | 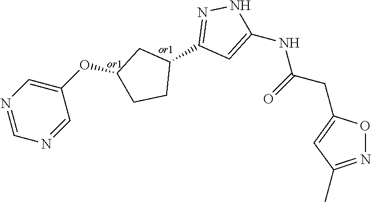 [SMILES: Cc1cc(CC(=O)Nc2cc([C@@H]3CC[C@H](Oc4cncnc4)C3)n[nH]2)on1] | ¹H NMR (400 MHz, DMSO-d₆) 1.63- 1.86 (m, 2H), 1.91 (t, J = 10.0 Hz, 1H), 2.07 (d, J = 10.1 Hz, 2H), 2.65 (dt, J = 14.4, 7.5 Hz, 1H), 3.15 (p, J = 9.0 Hz, 1H), 3.83 (s, 2H), 5.09 (q, J = 5.0, 3.8 Hz, 1H), 6.27 (d, J = 39.6 Hz, 2H), 8.53 (s, 2H), 8.79 (s, 1H), 10.64 (s, 1H), 12.17 (s, 1H) | 369.25 |
| 3-(methoxymethyl)-1-methyl- N-(5-((1S,3R)-3- phenoxycyclopentyl)-1H- pyrazol-3-yl)-1H-pyrazole-5- carboxamide | 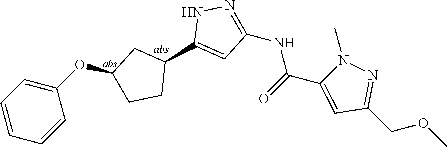 [SMILES: COCc1cc(C(=O)Nc2cc([C@H]3CC[C@@H](Oc4ccccc4)C3)[nH]n2)n(C)n1] | ¹H NMR (400 MHz, DMSO-d₆) 12.26 (d, J = 2.1 Hz, 1H), 10.72 (s, 1H), 7.34- 7.20 (m, 2H), 7.12 (s, 1H), 6.91 (dd, J = 8.4, 7.3 Hz, 3H), 6.45 (d, J = 2.2 Hz, 1H), 5.00-4.75 (m, 1H), 4.34 (s, 2H), 4.05 (s, 3H), 3.27 (s, 3H), 3.24-3.02 (m, 1H), 2.73-2.53 (m, 1H), 2.13-1.97 (m, 2H), 1.96-1.69 (m, 3H). | 396.30 |
| 3-(methoxymethyl)-1-methyl- N-(5-((1R,3S)-3- phenoxycyclopentyl)-1H- pyrazol-3-yl)-1H-pyrazole-5- carboxamide | 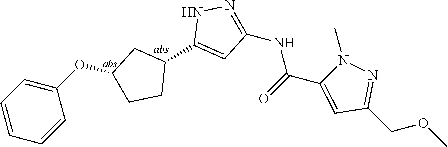 [SMILES: COCc1cc(C(=O)Nc2cc([C@@H]3CC[C@H](Oc4ccccc4)C3)[nH]n2)n(C)n1] | ¹H NMR (400 MHz, DMSO-d₆) 12.26 (s, 1H), 10.73 (s, 1H), 7.37-7.21 (m, 2H), 7.12 (s, 1H), 6.91 (dd, J = 8.4, 7.3 Hz, 3H), 6.44 (s, 1H), 4.99-4.80 (m, 1H), 4.34 (s, 2H), 4.05 (s, 3H), 3.27 (s, 3H), 3.14 (dq, J = 16.2, 8.3, 7.5 Hz, 1H), 2.71- 2.56 (m, 1H), 2.17- 1.96 (m, 2H), 1.94- 1.64 (m, 3H). | 396.35 |
| N-(3-((1S,3R)-3-(4-isopropyl- 2-oxo-2,3-dihydro-1H- imidazol-1-yl)cyclopentyl)- 1H-pyrazol-5-yl)-2-(3- methylisoxazol-5- yl)acetamide | 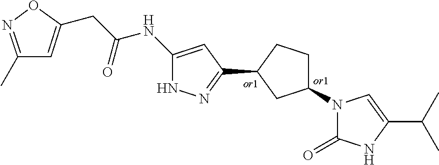 [SMILES: Cc1cc(CC(=O)Nc2cc([C@H]3CC[C@@H](n4cc(C(C)C)[nH]c4=O)C3)n[nH]2)on1] | ¹H NMR (400 MHz, DMSO-d₆) 12.19 (s, 1H), 10.65 (s, 1H), 9.96 (s, 1H), 6.35 (s, 1H), 6.22 (s, 1H), 6.16 (dd, J = 2.2, 1.2 Hz, 1H), 4.41 (p, J = 8.0 Hz, 1H), 3.83 (s, 2H), 2.52- 2.67 (m, 1H), 2.20 (s, 4H), 1.92-2.08 (m, 2H), 1.69-1.87 (m, 3H), 0.99-1.12 (m, 6H). | 399.15 |
| N-(3-((1R,3S)-3-(4-isopropyl- 2-oxo-2,3-dihydro-1H- imidazol-1-yl)cyclopentyl)- 1H-pyrazol-5-yl)-2-(3- methylisoxazol-5- yl)acetamide | 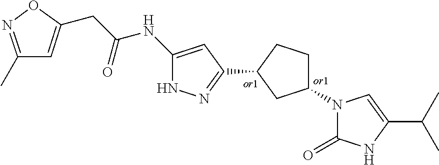 [SMILES: Cc1cc(CC(=O)Nc2cc([C@@H]3CC[C@H](n4cc(C(C)C)[nH]c4=O)C3)n[nH]2)on1] | ¹H NMR (400 MHz, DMSO-d₆) 12.19 (s, 1H), 10.65 (s, 1H), 9.96 (s, 1H), 6.36 (d, J = 2.2 Hz, 1H), 6.22 (s, 1H), 6.17 (s, 1H), 4.41 (p, J = 8.1 Hz, 1H), 3.83 (s, 2H), 3.13 (s, 1H), 2.52-2.58 (m, 1H), 2.20 (s, 4H), 1.94- 2.08 (m, 2H), 1.7- 1.87 (m, 3H), 1.09 (d, J = 6.8 Hz, 6H). | 399.20 |
| 2-(3-methylisoxazol-5-yl)-N- (5-((1R,3S)-3-((4- methylpyridin-3- yl)oxy)cyclopentyl)-1H- pyrazol-3-yl)acetamide | 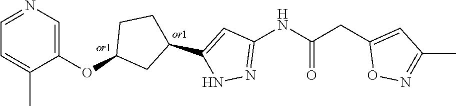 [SMILES: Cc1cc(CC(=O)Nc2cc([C@@H]3CC[C@H](Oc4cnccc4C)C3)[nH]n2)on1] | ¹H NMR (400 MHz, DMSO-d₆) 1.72- 1.94 (m, 3H), 1.94- 2.11 (m, 2H), 2.13 (s, 3H), 2.20 (s, 3H), 2.61 (ddd, J = 14.4, 8.5, 6.4 Hz, 1H), 3.14 (q, J = 8.7 Hz, 1H), 3.82 (s, 2H), 5- 5.06 (m, 1H), 6.22 (s, 1H), 6.31 (s, 1H), 7.17 (d, J = 4.6 Hz, 1H), 8.04 (d, J = 4.6 Hz, 1H), 8.21 (s, 1H), 10.63 (s, 1H), 12.16 (s, 1H). | 382.10 |
| 2-(3-methylisoxazol-5-yl)-N- (5-((1S,3R)-3-((4- methylpyridin-3- yl)oxy)cyclopentyl)-1H- pyrazol-3-yl)acetamide | 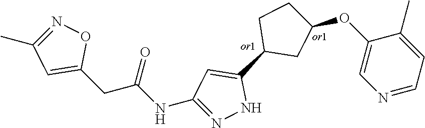 [SMILES: Cc1cc(CC(=O)Nc2cc([C@H]3CC[C@@H](Oc4cnccc4C)C3)[nH]n2)on1] | ¹H NMR (400 MHz, DMSO-d₆) 1.72- 1.93 (m, 3H), 1.97- 2.1 (m, 2H), 2.13 (s, 3H), 2.20 (s, 3H), 2.62 (dt, J = 14.5, 7.6 Hz, 1H), 3.09-3.2 (m, 1H), 3.82 (s, 2H), 5.03 (s, 1H), 6.22 (s, 1H), 6.31 (s, 1H), 7.19 (d, J = 4.7 Hz, 1H), 8.05 (d, J = 4.7 Hz, 1H), 8.22 (s, 1H), 10.65 (s, 1H), 12.17 (s, 1H). | 382.10 |
| N-(3-((1S,3R)-3-((4- cyclopropylpyridin-3- yl)oxy)cyclopentyl)-1H- pyrazol-5-yl)-2-(3- methylisoxazol-5- yl)acetamide | 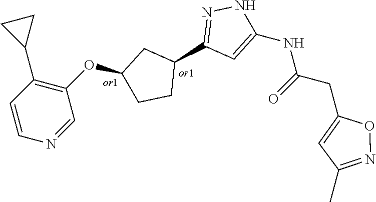 [SMILES: Cc1cc(CC(=O)Nc2cc([C@H]3CC[C@@H](Oc4cnccc4C4CC4)C3)n[nH]2)on1] | ¹H NMR (400 MHz, DMSO-d₆) 0.66- 0.74 (m, 2H), 0.91- 1.03 (m, 2H), 1.75- 1.95 (m, 3H), 1.97- 2.17 (m, 3H), 2.20 (s, 3H), 2.61 (ddd, J = 14.5, 8.7, 6.5 Hz, 1H), 3.1-3.23 (m, 1H), 3.82 (s, 2H), 5.03 (dd, J = 6.4, 3.6 Hz, 1H), 6.22 (s, 1H), 6.33 (s, 1H), 6.77 (d, J = 4.9 Hz, 1H), 8.03 (d, J = 4.9 Hz, 1H), 8.20 (s, 1H), 10.63 (s, 1H), 12.16 (s, 1H). | 408.35 |
| N-(3-((1R,3S)-3-((4- cyclopropylpyridin-3- yl)oxy)cyclopentyl)-1H- pyrazol-5-yl)-2-(3- methylisoxazol-5- yl)acetamide | 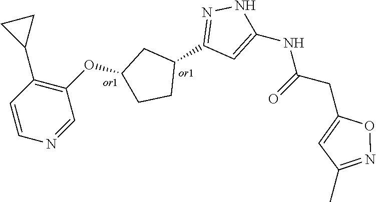 [SMILES: Cc1cc(CC(=O)Nc2cc([C@@H]3CC[C@H](Oc4cnccc4C4CC4)C3)n[nH]2)on1] | ¹H NMR (400 MHz, DMSO-d₆) 0.66- 0.74 (m, 2H), 0.91- 1.02 (m, 2H), 1.75- 1.86 (m, 2H), 1.86- 1.95 (m, 1H), 1.95- 2.17 (m, 3H), 2.20 (s, 3H), 2.56-2.67 (m, 1H), 3.15 (q, J = 8.4 Hz, 1H), 3.82 (s, 2H), 5.03 (dd, J = 6.6, 3.6 Hz, 1H), 6.22 (s, 1H), 6.34 (d, J = 2.2 Hz, 1H), 6.77 (d, J = 4.9 Hz, 1H), 8.03 (d, J = 4.9 Hz, 1H), 8.20 (s, 1H), 10.64 (s, 1H), 12.17 (s, 1H). | 408.20 |
| rel-3-(methoxymethyl)-1- methyl-N-(3-((1R,3S)-3-((5- methylpyridin-3- yl)oxy)cyclopentyl)-1H- pyrazol-5-yl)-1H-pyrazole-5- carboxamide | 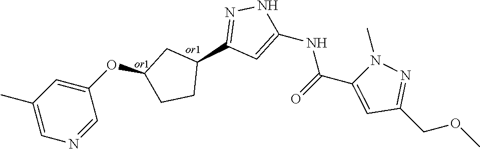 | ¹H NMR (400 MHz, DMSO-d₆) 1.67- 1.96 (m, 3H), 1.98- 2.18 (m, 2H), 2.6- 2.66 (m, 1H), 3.16 (q, J = 8.4 Hz, 1H), 3.27 (s, 3H), 4.05 (s, 3H), 4.34 (s, 2H), 4.88-5.08 (m, 1H), 6.45 (s, 1H), 6.94- 7.31 (m, 2H), 7.85- 8.28 (m, 2H), 10.73 (s, 1H), 12.26 (s, 1H). | 411.2 |
| rel-3-(methoxymethyl)-1- methyl-N-(3-((1R,3S)-3-((5- methylpyridin-3- yl)oxy)cyclopentyl)-1H- pyrazol-5-yl)-1H-pyrazole-5- carboxamide | 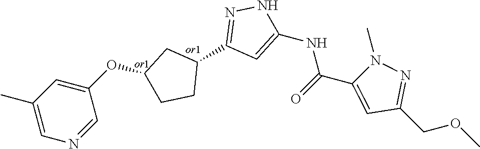 | ¹H NMR (400 MHz, DMSO-d₆) 1.67- 1.96 (m, 3H), 1.98- 2.18 (m, 2H), 2.6- 2.66 (m, 1H), 3.16 (q, J = 8.4 Hz, 1H), 3.27 (s, 3H), 4.05 (s, 3H), 4.34 (s, 2H), 4.88-5.08 (m, 1H), 6.45 (s, 1H), 6.94- 7.31 (m, 2H), 7.85- 8.28 (m, 2H), 10.73 (s, 1H), 12.26 (s, 1H). | 411.2 |
| 3-(methoxymethyl)-1-methyl- N-(5-((1S,3R)-3-((2- methylpyridin-3- yl)oxy)cyclopentyl)-1H- pyrazol-3-yl)-1H-pyrazole-5- carboxamide | 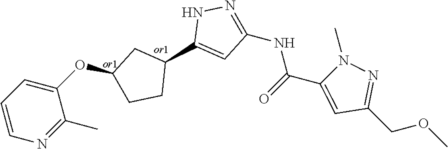 [SMILES: COCc1cc(C(=O)Nc2cc([C@H]3CC[C@@H](Oc4cccnc4C)C3)[nH]n2)n(C)n1] | ¹H NMR (400 MHz, DMSO-d₆) 12.28 (s, 1H), 10.75 (s, 1H), 7.98 (dd, J = 4.8, 1.3 Hz, 1H), 7.31 (dd, J = 8.4, 1.4 Hz, 1H), 7.07° C. 7.21 (m, 2H), 6.46 (s, 1H), 4.95 (d, J = 6.1 Hz, 1H), 4.34 (s, 2H), 4.05 (s, 3H), 3.27 (s, 3H), 3.14-3.23 (m, 1H), 2.57-2.67 (m, 1H), 2.33 (s, 3H), 2.08 (s, 2H), 1.7- 1.94 (m, 3H). | 411.15 |
| 3-(methoxymethyl)-1-methyl- N-(5-((1R,3S)-3-((2- methylpyridin-3- yl)oxy)cyclopentyl)-1H- pyrazol-3-yl)-1H-pyrazole-5- carboxamide | 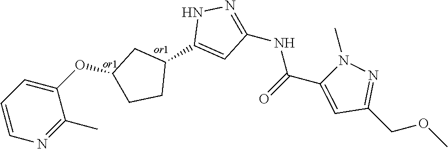 [SMILES: COCc1cc(C(=O)Nc2cc([C@@H]3CC[C@H](Oc4cccnc4C)C3)[nH]n2)n(C)n1] | ¹H NMR (400 MHz, DMSO-d₆) 12.28 (s, 1H), 10.75 (s, 1H), 7.98 (dd, J = 4.8, 1.3 Hz, 1H), 7.31 (dd, J = 8.4, 1.4 Hz, 1H), 7.1-7.21 (m, 2H), 6.45 (s, 1H), 4.95 (t, J = 3.4 Hz, 1H), 4.34 (s, 2H), 4.05 (s, 3H), 3.27 (s, 3H), 3.14-3.23 (m, 1H), 2.57-2.67 (m, 1H), 2.33 (s, 3H), 2.07 (s, 2H), 1.71- 1.96 (m, 3H). | 411.15 |
| rel-3-(methoxymethyl)-1- methyl-N-(3-((1R,3S)-3- (pyrimidin-5- yloxy)cyclopentyl)-1H- pyrazol-5-yl)-1H-pyrazole-5- carboxamide | 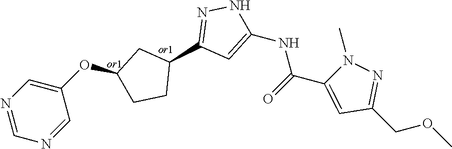 [SMILES: COCc1cc(C(=O)Nc2cc([C@H]3CC[C@@H](Oc4cncnc4)C3)n[nH]2)n(C)n1] | ¹H NMR (400 MHz, DMSO-d₆) 1.74° C. 1.88 (m, 1H), 1.93 (d, J = 13.0 Hz, 2H), 2.02° C. 2.15 (m, 2H), 2.69 (dt, J = 14.6, 7.6 Hz, 1H), 3.12° C. 3.23 (m, 1H), 3.27 (s, 3H), 4.05 (s, 3H), 4.34 (s, 2H), 5.10 (s, 1H), 6.46 (s, 1H), 7.12 (s, 1H), 8.55 (s, 2H), 8.80 (s, 1H), 10.74 (s, 1H), 12.28 (s, 1H). | 398.30 |
| rel-3-(methoxymethyl)-1- methyl-N-(3-((1R,3S)-3- (pyrimidin-5- yloxy)cyclopentyl)-1H- pyrazol-5-yl)-1H-pyrazole-5- carboxamide | 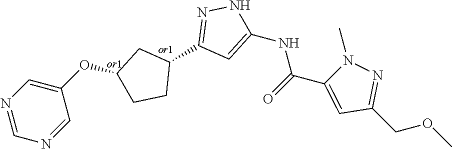 [SMILES: COCc1cc(C(=O)Nc2cc([C@@H]3CC[C@H](Oc4cncnc4)C3)n[nH]2)n(C)n1] | ¹H NMR (400 MHz, DMSO-d₆) 1.82- 1.93 (d, J = 13.2 Hz, 3H), 2.12 (d, J = 6.5 Hz, 2H), 2.71 (t, J = 7.2 Hz, 1H), 3.18 (m, 1H), 3.27 (s, 3H), 4.05 (s, 3H), 4.34 (s, 2H), 5.10 (s, 1H), 6.46 (s, 1H), 7.12 (s, 1H), 8.55 (s, 2H), 8.80 (s, 1H), 10.74 (s, 1H), 12.28 (s, 1H). | 398.30 |
| N-(5-((1S,3R)-3-(2- isopropylphenoxy)cyclopentyl)- 1H-pyrazol-3-yl)-2-(3- methylisoxazol-5- yl)acetamide | 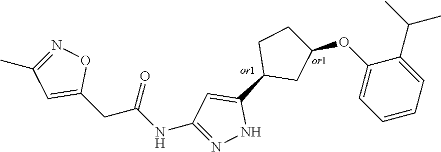 [SMILES: Cc1cc(CC(=O)Nc2cc([C@H]3CC[C@@H](Oc4ccccc4C(C)C)C3)[nH]n2)on1] | ¹H NMR (400 MHz, DMSO-d₆) 1.12 (dd, J = 6.9, 4.6 Hz, 6H), 1.69-1.83 (m, 2H), 1.83-1.93 (m, 1H), 1.93-2.12 (m, 2H), 2.20 (s, 3H), 2.60 (dt, J = 14.6, 7.3 Hz, 1H), 3.07-3.25 (m, 2H), 3.82 (s, 2H), 4.87-4.93 (m, 1H), 6.22 (s, 1H), 6.31 (s, 1H), 6.82-6.93 (m, 2H), 7.07-7.19 (m, 2H), 10.63 (s, 1H), 12.15 (s, 1H). | 409.15 |
| N-(5-((1R,3S)-3-(2- isopropylphenoxy)cyclopentyl)- 1H-pyrazol-3-yl)-2-(3- methylisoxazol-5- yl)acetamide | 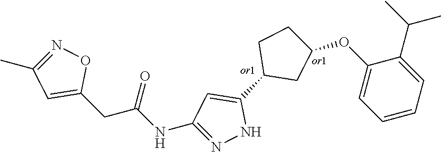 [SMILES: Cc1cc(CC(=O)Nc2cc([C@@H]3CC[C@H](Oc4ccccc4C(C)C)C3)[nH]n2)on1] | ¹H NMR (400 MHz, DMSO-d₆) 1.12 (dd, J = 6.9, 4.6 Hz, 6H), 1.69-1.84 (m, 2H), 1.84-1.93 (m, 1H), 1.94-2.11 (m, 2H), 2.20 (s, 3H), 2.55-2.66 (m, 1H), 3.07-3.25 (m, 2H), 3.82 (s, 2H), 4.87- 4.93 (m, 1H), 6.22 (s, 1H), 6.31 (d, J = 2.3 Hz, 1H), 6.82- 6.93 (m, 2H), 7.07- 7.19 (m, 2H), 10.63 (s, 1H), 12.13-12.18 (m, 1H). | 409.10 |

### Example 11

### (1R,3S)-3-(3-((2-(methoxymethyl)thiazolo[5,4-c]pyridin-6-yl)amino)-1H-pyrazol-5-yl)cyclopentyl isopropylcarbamate and (1S,3R)-3-(3-((2-(methoxymethyl)thiazolo[5,4-c]pyridin-6-yl)amino)-1H-pyrazol-5-yl)cyclopentyl isopropylcarbamate

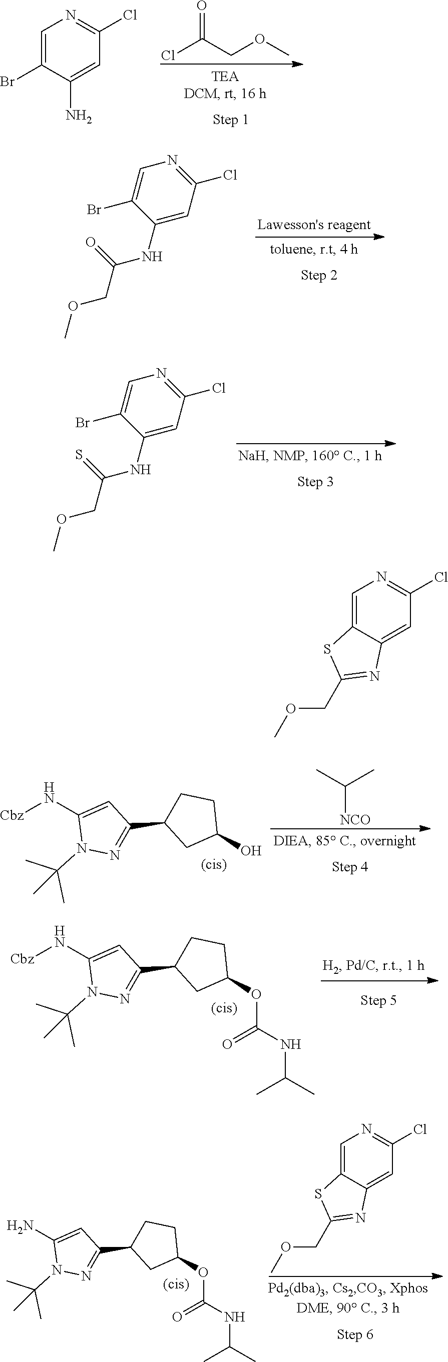

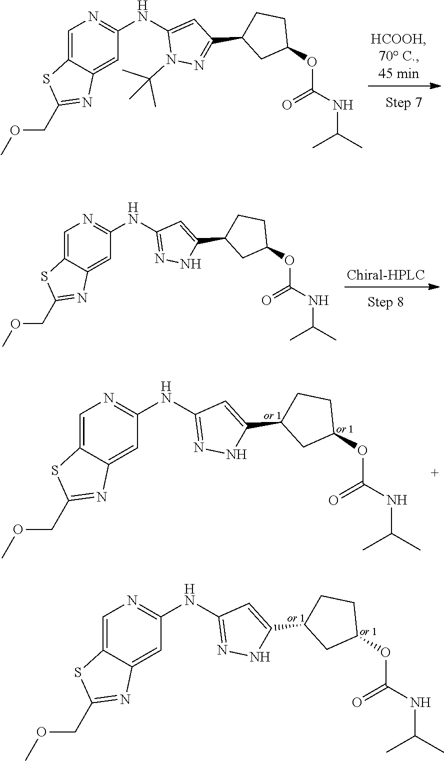

### N-(5-bromo-2-chloropyridin-4-yl)-2-methoxyacetamide

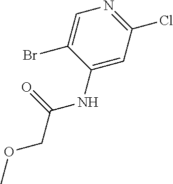

Step 1: To an ice cold solution of 5-bromo-2-chloropyridin-4-amine (5 g, 1 eq, 24 mmol) in DCM (50 mL), TEA (3.6 g, 1.5 eq, 36 mmol) and 2-methoxyacetyl chloride (3.9 g, 1.5 eq, 36 mmol) were added. The mixture was stirred at 20° C. for 16 h. The solvent was removed under reduced pressure, and the residue was taken up in water (70 mL) and extracted with EtOAc (3×50 mL). The combined organic extracts were washed with brine, dried over Na₂SO₄, filtered, and concentrated under reduced pressure. The residue was purified by flash (Mobile Phase A: Water (10 mmol/L NH₄HCO₃), Mobile Phase B: ACN; Flow rate: 60 mL/min; Gradient: 45% B to 55% B in 7 min); After solvent evaporation afforded N-(2,5-dichloropyridin-4-yl)-2-methoxyacetamide (1.51 g, 5.4 mmol, 22%) as a light yellow solid.

m/z (ES⁺) [M+H]⁺=278.85; HPLC tR=0.997 min.

### N-(5-bromo-2-chloropyridin-4-yl)-2-methoxyethanethioamide

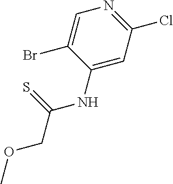

Step 2: To a solution of N-(5-bromo-2-chloropyridin-4-yl)-2-methoxyacetamide (1.33 g, 1 eq, 4.76 mmol) in toluene (20 mL) was added 2,4-bis(4-methoxyphenyl)-1,3,2,4-dithiadiphosphetane 2,4-disulfide (1.44 g, 0.75 eq, 3.57 mmol). The mixture was heated at 110° C. under N₂ for 4 h. The reaction mixture was filtered to remove the filter cake and the filtrate was concentrated in vacuo. The resulting crude material was purified by silica gel chromatography eluting with a mixture petroleum ether/ethyl acetate 20:1 to afford 2-(1-benzothiophen-2-yl)-N-(1-{5-[(3S)-1-methylpiperidin-3-yl]pyrimidin-2-yl}-1H-pyrazol-4-yl)acetamide (1.1 g, 4.76 mmol, 78%) as a yellow amorphous solid.

m/z (ES⁺) [M+H]⁺=294.90; HPLC tR=0.727 min.

### 6-chloro-2-(methoxymethyl)thiazolo[5,4-c]pyridine

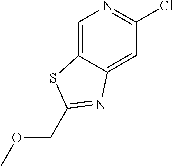

Step 3: To a solution of N-(5-bromo-2-chloropyridin-4-yl)-2-methoxyethanethioamide (490 mg, 1 eq, 1.66 mmol) in NMP (5 mL), was added NaH (59.7 mg, 0.9 eq, 1.49 mmol). The mixture was heated under 160° C. for 1 hour. The reaction mixture was allowed to reach room temperature and poured into ice cold water, followed by extraction with EtOAc (3×40 mL). The combined organic extracts were washed with brine, dried over Na₂SO₄, filtered and concentrated under reduced pressure. The crude residue was purified by flash (Mobile Phase A: Water, Mobile Phase B: ACN; Flow rate:60 mL/min; Gradient:40 B to 50 B in 8 min); After solvent evaporation afforded 6-chloro-2-(methoxymethyl)thiazolo[5,4-c]pyridine (240 mg, 1.12 mmol, 67.4%) as brown solid.

m/z (ES⁺) [M+H]⁺=215.00; HPLC tR=0.594 min.

### cis-3-(5-(((benzyloxy)carbonyl)amino)-1-(tert-butyl)-1H-pyrazol-3-yl)cyclopentyl isopropylcarbamate

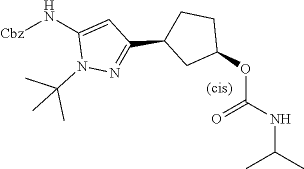 [SMILES: CC(C)NC(=O)O[C@@H]1CC[C@H](c2cc(NC(=O)OCc3ccccc3)n(C(C)(C)C)n2)C1]

Step 4: To a solution of benzyl (1-(tert-butyl)-5-(cis-3-hydroxycyclopentyl)-1H-pyrazol-3-yl) carbamate (5 g, 1 eq, 0.01 mol) in toluene (20 mL) was added 2-isocyanatopropane (6 g, 5 eq, 0.07 mol), DIEA (5 g, 3 eq, 0.04 mol). The mixture was stirred at 85° C. for 16 hours. The mixture was diluted with water, and the aqueous phase was extracted with EA (3×40 mL). The combined organic layers were washed with brine, dried over sodium sulfate, filtered, and concentrated in vacuo. The resulting crude material was purified by Flash (Mobile Phase A: water/0.1% formic acid, Mobile Phase B: ACN; Flow rate:60 mL/min; Gradient:40 B to 50 B in 8 min); After solvent evaporation afforded cis-3-(3-(((benzyloxy)carbonyl) amino)-1-(tert-butyl)-1H-pyrazol-5-yl) cyclopentyl isopropylcarbamate (4.2 g, 8.9 mmol, 60%) as a brown amorphous solid.

m/z (ES⁺) [M+H]⁺=443.10; HPLC tR=0.889 min.

### cis-3-(5-amino-1-(tert-butyl)-1H-pyrazol-3-yl)cyclopentyl isopropylcarbamate

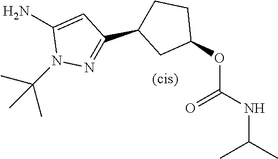 [SMILES: CC(C)NC(=O)O[C@@H]1CC[C@H](c2cc(N)n(C(C)(C)C)n2)C1]

Step 5: A solution of cis-3-(3-(((benzyloxy)carbonyl) amino)-1-(tert-butyl)-1H-pyrazol-5-yl) cyclopentyl isopropylcarbamate (4.2 g, 1 eq, 9.5 mmol) in MeOH (15 mL) was bubbling nitrogen through the reaction mixture for 3 times. Then Pd/C (0.50 g, 0.5 eq, 4.7 mmol) was added. After bubbling H₂ through the reaction mixture for 3 times, the mixture was stirred at room temperature for 2 hours with H₂. The reaction mixture was filtered to remove the filter cake and the filtrate was concentrated in vacuo. The resulting crude material was purified by Flash (Mobile Phase A: water/0.1% formic acid, Mobile Phase B: ACN; Flow rate:60 mL/min; Gradient:45 B to 55 B in 6 min); After solvent evaporation afforded cis-3-(3-amino-1-(tert-butyl)-1H-pyrazol-5-yl) cyclopentyl isopropylcarbamate (2.3 g, 7.5 mmol, 79%) as a brown amorphous oil.

m/z (ES⁺) [M+H]⁺=309.15; HPLC tR=0.691 min.

### cis-3-(1-(tert-butyl)-5-((2-(methoxymethyl)thiazolo[5,4-c]pyridin-6-yl)amino)-1H-pyrazol-3-yl)cyclopentyl isopropylcarbamate

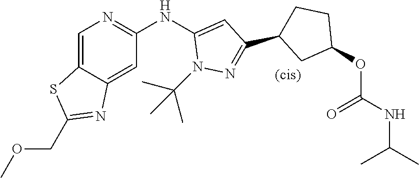 [SMILES: COCc1nc2cc(Nc3cc([C@H]4CC[C@@H](OC(=O)NC(C)C)C4)nn3C(C)(C)C)ncc2s1]

Step 6: 6-chloro-2-(methoxymethyl) thiazolo[5,4-c]pyridine (234 mg, 1.2 eq, 1.09 mmol), Cs₂CO₃ (1.18 g, 4 eq, 3.63 mmol) and Xphos (86.4 mg, 0.2 eq, 182 μmol) were added to a solution of cis-3-(3-amino-1-(tert-butyl)-1H-pyrazol-5-yl) cyclopentyl isopropylcarbamate (280 mg, 1 eq, 908 μmol) in DME (4 mL). After bubbling nitrogen through the reaction mixture for 5 minutes, Pd₂dba₃ (166 mg, 0.2 eq, 182 μmol) was added. The reaction mixture is heated at 90° C. for 3 hours with vigorous stirring. After cooling to room temperature, the mixture was evaporated and extracted with ethyl acetate (3×50 mL), dried over Na₂SO₄ and evaporated in vacuo. The crude residue was purified by flash (Mobile Phase A: Water, Mobile Phase B: ACN; Flow rate: 60 mL/min; Gradient: 45% B to 55% B in 6 min); After solvent evaporation afforded cis-3-(1-(tert-butyl)-3-((2-(methoxymethyl)thiazolo[5,4-c]pyridin-6-yl)amino)-1H-pyrazol-5-yl)cyclopentyl isopropylcarbamate (120 mg, 247 μmol, 27.2%) as brown oil.

m/z (ES⁺) [M+H]⁺=487.10; HPLC tR=1.029 min.

### (1R,3S)-3-(3-((2-(methoxymethyl)thiazolo[5,4-c]pyridin-6-yl)amino)-1H-pyrazol-5-yl)cyclopentyl isopropylcarbamate

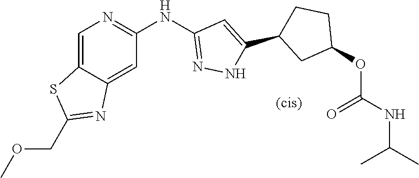 [SMILES: COCc1nc2cc(Nc3cc([C@H]4CC[C@@H](OC(=O)NC(C)C)C4)[nH]n3)ncc2s1]

Step 7: The solution of cis-3-(1-(tert-butyl)-3-((2-(methoxymethyl) thiazolo[5,4-c]pyridin-6-yl) amino)-1H-pyrazol-5-yl) cyclopentyl isopropylcarbamate (101 mg, 1 Eq, 208 μmol) in FA (2 mL) was heated under 70° C. for 45 min. After cooling to room temperature, the mixture was evaporated. The crude residue was purified by Prep-HPLC (Column: Sunfire prep C18 column, 30*150 mm, 5 m; Mobile Phase A: Water (0.05% TFA), Mobile Phase B: ACN; Flow rate: 60 mL/min; Gradient: 10% B to 45% B in 7 min); After solvent evaporation afforded cis-3-(3-((2-(methoxymethyl) thiazolo[5,4-c]pyridin-6-yl) amino)-1H-pyrazol-5-yl) cyclopentyl isopropylcarbamate (27 mg, 63 μmol, 30%) as a yellow solid (1R,3S)-3-(3-((2-(methoxymethyl)thiazolo[5,4-c]pyridin-6-yl)amino)-1H-pyrazol-5-yl)cyclopentyl isopropylcarbamate and (1S,3R)-3-(3-((2-(methoxymethyl)thiazolo[5,4-c]pyridin-6-yl)amino)-1H-pyrazol-5-yl)cyclopentyl isopropylcarbamate

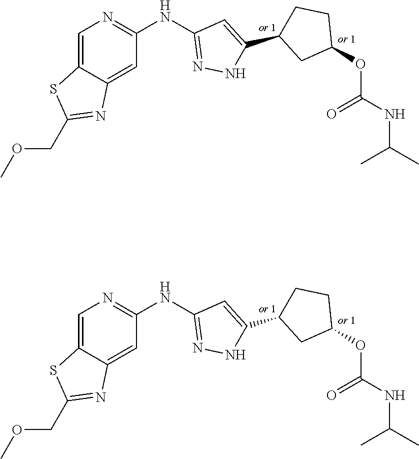

Step 8: cis-3-(3-((2-(methoxymethyl)thiazolo[5,4-c]pyridin-6-yl)amino)-1H-pyrazol-5-yl)cyclopentyl isopropylcarbamate (27 mg, 1 eq, 63 μmol) was purified by Chiral-HPLC (Column: CHIRALPAK IG, 2*25 cm, 5 m; Mobile Phase A: Hex (0.2% DEA)—HPLC, Mobile Phase B: EtOH: DCM=1:1—HPLC; Flow rate: 20 mL/min; Gradient: 80% B to 80% B in 14.5 min; Wave Length: 220/254 nm; RT1(min): 9.73; RT2(min): 12.26; Sample Solvent: EtOH: DCM=1:1—HPLC; Injection Volume: 0.9 mL); Lyophilization yielded (1R,3S)-3-(3-((2-(methoxymethyl)thiazolo[5,4-c]pyridin-6-yl)amino)-1H-pyrazol-5-yl)cyclopentyl isopropylcarbamate (6 mg, 0.01 mmol, 40%) as a light yellow amorphous solid.

m/z (ES⁺) [M+H]⁺=431.15; HPLC tR=0.731 min.

¹H NMR (400 MHz, Chloroform-d) δ 8.77 (s, 1H), 7.77 (s, 1H), 7.28 (s, 1H), 6.02 (s, 1H), 5.24 (s, 1H), 4.86 (s, 2H), 4.70 (s, 1H), 3.83 (s, 1H), 3.60 (s, 3H), 3.22 (t, J=8.3 Hz, 1H), 2.53 (s, 1H), 2.16 (d, J=5.5 Hz, 1H), 1.97 (s, 2H), 1.90 (d, J=10.1 Hz, 2H), 1.28 (s, 1H), 1.18 (dd, J=6.7, 2.5 Hz, 6H).

Lyophilization yielded (1S,3R)-3-(3-((2-(methoxymethyl)thiazolo[5,4-c]pyridin-6-yl)amino)-1H-pyrazol-5-yl)cyclopentyl isopropylcarbamate (8.4 mg, 20 μmol, 62%) as a light yellow amorphous solid.

m/z (ES⁺) [M+H]⁺=431.10; HPLC tR=0.724 min.

¹H NMR (400 MHz, Chloroform-d) δ 8.78 (s, 1H), 7.76 (s, 1H), 7.35 (s, 1H), 6.03 (s, 1H), 5.24 (s, 1H), 4.86 (s, 2H), 4.71 (s, 1H), 3.83 (s, 1H), 3.60 (s, 3H), 3.26-3.18 (m, 1H), 2.54 (s, 1H), 2.18-2.12 (m, 1H), 1.96 (s, 2H), 1.90 (d, J=9.5 Hz, 2H), 1.28 (s, 1H), 1.18 (dd, J=6.8, 2.9 Hz, 6H).

Additional compounds prepared according to the methods of Example 11 are depicted in Table 7 below.

TABLE 7
Additional Exemplary Compounds

| | | | MS |
|---|---|---|---|
| | | | [M + |
| Compound | Structure | Proton NMR | 1] |
| (1R,3S)-3-(3- (thiazolo[5,4- c]pyridin-4- ylamino)- 1H-pyrazol-5- yl)cyclopentyl isopropylcarbamate | 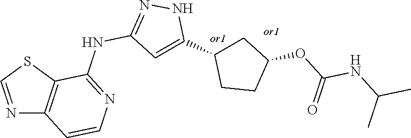 [SMILES: CC(C)NC(=O)O[C@@H]1CC[C@H](c2cc(Nc3nccc4ncsc34)n[nH]2)C1] | ¹H NMR (400 MHz, Chloroform-d) 9.14 (s, 1H), 8.25 (d, J = 5.9 Hz, 1H), 7.57 (d, J = 5.9 Hz, 1H), 6.47 (s, 1H), 5.24 (s, 1H), 4.68 (s, 1H), 3.83 (s, 1H), 3.25 (p, J = 8.0 Hz, 1H), 2.54 (dd, J = 14.5, 7.4 Hz, 1H), 2.21- 2.14 (m, 1H), 1.98 (s, 4H), 1.92 (d, J = 8.5 Hz, 1H), 1.17 (t, J = 5.8 Hz, 6H). | 387.25 |
| (1S,3R)-3-(3- (thiazolo[5,4- c]pyridin-4- ylamino)- 1H-pyrazol-5- yl)cyclopentyl isopropylcarbamate | 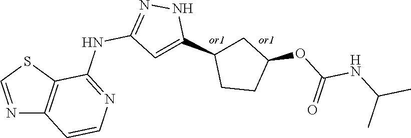 [SMILES: CC(C)NC(=O)O[C@H]1CC[C@@H](c2cc(Nc3nccc4ncsc34)n[nH]2)C1] | ¹H NMR (400 MHz, Chloroform-d) 9.13 (s, 1H), 8.26 (d, J = 5.8 Hz, 1H), 7.57 (d, J = 5.8 Hz, 1H), 6.46 (s, 1H), 5.24 (s, 1H), 4.66 (s, 1H), 3.83 (s, 1H), 3.25 (p, J = 8.2 Hz, 1H), 2.55 (dt, J = 14.7, 7.9 Hz, 1H), 2.22- 2.14 (m, 1H), 1.98 (s, 4H), 1.92 (d, J = 8.5 Hz, 1H), 1.17 (t, J = 5.9 Hz, 6H). | 387.25 |
| N-isopropyl-2- ((1R,3S)-3-(3- (pyrazolo[1,5-a] pyrazin-4-ylamino)- 1H-pyrazol-5-yl) cyclopentyl) acetamide | 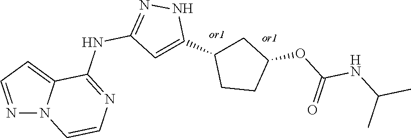 [SMILES: CC(C)NC(=O)O[C@@H]1CC[C@H](c2cc(Nc3nccn4nccc34)n[nH]2)C1] | ¹H NMR (400 MHz, Chloroform-d) 7.94 (d, J = 2.4 Hz, 2H), 7.42 (s, 1H), 6.83 (s, 1H), 6.60 (s, 1H), 5.24 (s, 1H), 4.68 (s, 1H), 3.83 (s, 1H), 3.25 (p, J = 8.2 Hz, 1H), 2.55 (dt, J = 15.1, 8.0 Hz, 1H), 2.18 (d, J = 8.0 Hz, 1H), 2.00-1.91 (m, 5H), 1.16 (t, J = 6.6 Hz, 6H). | 370.15 |
| (1S,3R)-3-(3- (pyrazolo[1,5-a] pyrazin-4-ylamino)- 1H-pyrazol-5- yl)cyclopentyl isopropylcarbamate | 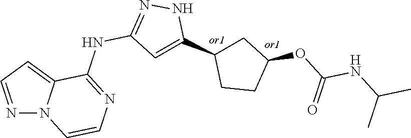 [SMILES: CC(C)NC(=O)O[C@H]1CC[C@@H](c2cc(Nc3nccn4nccc34)n[nH]2)C1] | ¹H NMR (400 MHz, Chloroform-d) 7.96 (d, J = 2.4 Hz, 1H), 7.93 (s, 1H), 7.40 (s, 1H), 6.95 (s, 1H), 6.62 (s, 1H), 5.24 (s, 1H), 4.71 (s, 1H), 3.83 (s, 1H), 3.25 (p, J = 8.1 Hz, 1H), 2.55 (dd, J = 14.7, 7.6 Hz, 1H), 2.18 (d, J = 7.1 Hz, 1H), 1.95 (d, J = 18.1 Hz, 5H), 1.18 (dd, J = 6.7, 4.7 Hz, 6H). | 370.20 |
| (cis)-3-(3- (oxazolo[5,4-b] pyridin-2-ylamino)- 1H-pyrazol-5- yl)cyclopentyl isopropylcarbamate | 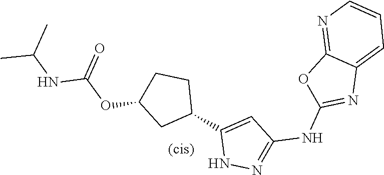 [SMILES: CC(C)NC(=O)O[C@@H]1CC[C@H](c2cc(Nc3nc4cccnc4o3)n[nH]2)C1] | ¹H NMR (400 MHz, DMSO-d₆) 12.18 (s, 1H), 11.05 (s, 1H), 7.94 (d, J = 5.0 Hz, 1H), 7.74 (d, J = 7.7 Hz, 1H), 7.24 (dd, J = 7.6, 5.0 Hz, 1H), 6.95 (d, J = 7.9 Hz, 1H), 6.43 (s, 1H), 5.01 (s, 1H), 3.57 (dq, J = 13.5, 6.7 Hz, 1H), 3.11-3.07 (m, 1H), 2.54-2.51 (m, 1H), 2.07-2.03 (m, 1H), 1.92-1.88 (m, 1H), 1.78-1.71 (m, 2H), 1.65-1.61 (m, 1H), 1.02 (dd, J = 6.6, 2.5 Hz, 6H). | 371.2 |
| rel-(1R,3S)-3- (5-amino-1H- pyrazol-3-yl) cyclopentyl isopropylcarbamate | 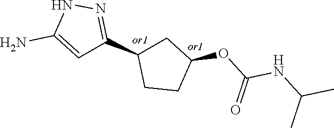 [SMILES: CC(C)NC(=O)O[C@H]1CC[C@@H](c2cc(N)[nH]n2)C1] | ¹H NMR (400 MHz, DMSO-d₆) 11.04 (s, 1H), 6.94 (d, J = 7.8 Hz, 1H), 5.19 (s, 1H), 4.96 (d, J = 7.4 Hz, 1H), 4.49 (s, 2H), 3.57 (h, J = 6.6 Hz, 1H), 2.91 (dt, J = 16.6, 7.3 Hz, 1H), 2.38 (dd, J = 13.8, 7.2 Hz, 1H), 1.95-1.81 (m, 2H), 1.71-1.49 (m, 3H), 1.03 (d, J = 6.5 Hz, 6H). | 253.05 |
| rel-(1R,3S)-3- (5-amino-1H- pyrazol-3-yl) cyclopentyl isopropylcarbamate | 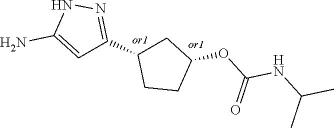 [SMILES: CC(C)NC(=O)O[C@@H]1CC[C@H](c2cc(N)[nH]n2)C1] | ¹H NMR (400 MHz, DMSO-d₆) 11.09 (s, 1H), 6.93 (d, J = 7.7 Hz, 1H), 5.21 (s, 1H), 4.96 (s, 2H), 4.33 (s, 1H), 3.62-3.52 (m, 1H), 2.90 (s, 1H), 2.37 (s, 1H), 2.00-1.79 (m, 2H), 1.63 (dd, J = 33.1, 23.0 Hz, 3H), 1.03 (d, J = 6.6 Hz, 6H). | 253.00 |
| rel-(1R,3S)-3- (3-formamido- 1H-pyrazol-5- yl)cyclopentyl isopropylcarbamate | 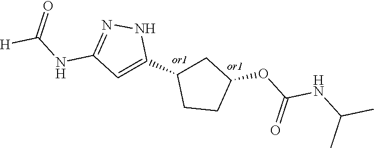 [SMILES: CC(C)NC(=O)O[C@@H]1CC[C@H](c2cc(NC=O)n[nH]2)C1] | ¹H NMR (400 MHz, DMSO-d₆) 12.11 (s, 1H), 10.51-9.91 (m, 1H), 8.86-8.03 (m, 1H), 6.95 (d, J = 7.8 Hz, 1H), 6.45-5.50 (dd, J = 201.5, 2.2 Hz, 1H), 4.99 (s, 1H), 3.62- 3.53 (m, 1H), 3.04 (s, 1H), 2.44 (d, J = 7.2 Hz, 1H), 2.33 (p, J = 1.9 Hz, 1H), 2.10-1.96 (m, 1H), 1.94-1.81 (m, 2H), 1.70 (t, J = 10.8 Hz, 1H), 1.03 (d, J = 6.5 Hz, 6H). | 281.05 |
| rel-(1R,3S)-3- (3-formamido- 1H-pyrazol-5- yl)cyclopentyl isopropylcarbamate | 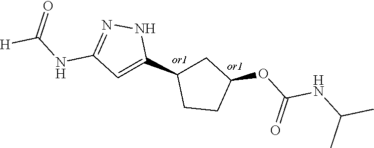 [SMILES: CC(C)NC(=O)O[C@H]1CC[C@@H](c2cc(NC=O)n[nH]2)C1] | ¹H NMR (400 MHz, DMSO-d₆) 12.11 (s, 1H), 10.50-9.89 (m, 1H), 8.74-7.96 (m, 1H), 6.95 (d, J = 7.8 Hz, 1H), 6.68-5.70 (m, 1H), 4.99 (s, 1H), 3.62-3.53 (m, 1H), 3.09- 3.00 (m, 1H), 2.46-2.29 (m, 1H), 2.08-1.96 (m, 1H), 1.94-1.81 (m, 1H), 1.81-1.63 (m, 2H), 1.58 (s, 1H), 1.03 (d, J = 6.6 Hz, 6H). | 281.05 |
| rel-ethyl 4-((3- ((1R,3S)-3-((iso- propylcarbamoyl) oxy)cyclopentyl)- 1H-pyrazol-5- yl)amino)-6- methylpyrazolo [1,5-a]pyrazine- | 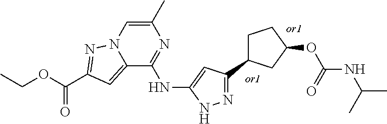 [SMILES: CCOC(=O)c1cc2c(Nc3cc([C@H]4CC[C@@H](OC(=O)NC(C)C)C4)n[nH]3)nc(C)cn2n1] | ¹H NMR (400 MHz, DMSO-d₆) 12.14 (s, 1H), 10.21 (s, 1H), 8.01 (s, 1H), 7.83 (s, 1H), 6.95 (d, J = 7.9 Hz, 1H), 6.71 (s, 1H), 5.02 (s, 1H), 4.34 (q, J = 7.1 Hz, 2H), 3.60-3.52 (m, 1H), 3.30 (s, 1H), 3.10 (s, 1H), 2.32 (s, 3H), 2.05 (s, 1H), 1.93 (s, 1H), 1.75 (s, 2H), 1.66 (s, 1H), 1.33 (t, J = 7.1 Hz, 3H), 1.06- 1.00 (m, 6H). | 456.20 |
| 2-carboxylate | | | |
| rel-ethyl 4-((3- ((1R,3S)-3-((iso- propylcarbamoyl) oxy)cyclopentyl)- 1H-pyrazol-5- yl)amino)-6- methylpyrazolo [1,5-a]pyrazine- | 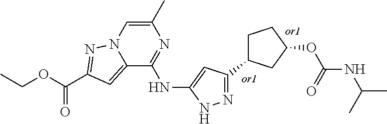 [SMILES: CCOC(=O)c1cc2c(Nc3cc([C@@H]4CC[C@H](OC(=O)NC(C)C)C4)n[nH]3)nc(C)cn2n1] | ¹H NMR (400 MHz, DMSO-d₆) 12.14 (s, 1H), 10.21 (s, 1H), 8.01 (s, 1H), 7.84 (s, 1H), 6.95 (d, J = 7.9 Hz, 1H), 6.72 (s, 1H), 5.02 (s, 1H), 4.34 (q, J = 7.1 Hz, 2H), 3.60-3.54 (m, 1H), 3.30 (s, 1H), 3.10 (s, 1H), 2.32 (s, 3H), 2.04 (d, J = 11.6 Hz, 1H), 1.92 (d, J = 15.4 Hz, 1H), 1.75 (d, J = 8.5 Hz, 2H), 1.66 (s, 1H), 1.34 (t, J = 7.1 Hz, 3H), 1.03 (dd, | 456.20 |
| 2-carboxylate | | J = 6.7, 3.3 Hz, 6H). | |
| rel-(1R,3S)- 3-(3-((2- (trifluoromethyl) pyrazolo[1,5- a]pyrazin- 4-yl)amino)-1H- pyrazol-5-yl) cyclopentyl isopropylcarbamate | 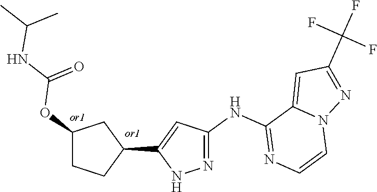 [SMILES: CC(C)NC(=O)O[C@@H]1CC[C@H](c2cc(Nc3nccn4nc(C(F)(F)F)cc34)n[nH]2)C1] | ¹H NMR (400 MHz, DMSO-d₆) 12.21 (s, 1H), 10.33 (s, 1H), 8.22 (d, J = 4.8 Hz, 1H), 7.83 (s, 1H), 7.62 (d, J = 4.8 Hz, 1H), 6.98 (d, J = 7.7 Hz, 1H), 6.67 (s, 1H), 5.02 (s, 1H), 3.58 (q, J = 6.8 Hz, 1H), 3.34 (s, 1H), 3.11 (s, 1H), 2.14-1.84 (m, 2H), 1.84-1.56 (m, 3H), 1.03 (dd, J = 6.7, 2.9 Hz, 6H). | 438.35 |
| rel-(1R,3S)- 3-(3-((2- (trifluoromethyl) pyrazolo[1,5- a]pyrazin- 4-yl)amino)-1H- pyrazol-5-yl) cyclopentyl isopropylcarbamate | 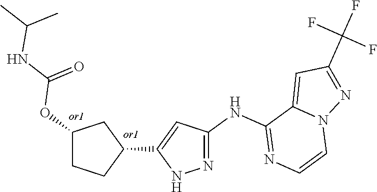 [SMILES: CC(C)NC(=O)O[C@H]1CC[C@@H](c2cc(Nc3nccn4nc(C(F)(F)F)cc34)n[nH]2)C1] | ¹H NMR (400 MHz, DMSO-d₆) 12.22 (s, 1H), 10.34 (s, 1H), 8.22 (d, J = 4.8 Hz, 1H), 7.82 (s, 1H), 7.62 (d, J = 4.9 Hz, 1H), 6.98 (d, J = 7.8 Hz, 1H), 6.66 (s, 1H), 5.02 (s, 1H), 3.63-3.54 (m, 1H), 3.34 (s, 1H), 3.15- 3.06 (m, 1H), 2.10-2.01 (m, 1H), 1.92 (s, 1H), 1.75 (d, J = 9.4 Hz, 2H), 1.65 (s, 1H), 1.04 (dd, J = 6.7, 3.0 Hz, 6H). | 438.35 |
| rel-(1R,3S)-3-(5- ((7,7-dimethyl-5- oxo-6,7-dihydro- 5H-pyrrolo[3,4- b]pyridin-2- yl)amino)-1H- pyrazol-3- yl)cyclopentyl isopropylcarbamate | 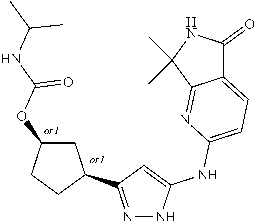 [SMILES: CC(C)NC(=O)O[C@@H]1CC[C@H](c2cc(Nc3ccc4c(n3)C(C)(C)NC4=O)[nH]n2)C1] | ¹H NMR (400 MHz, DMSO-d₆) 11.97 (s, 1H), 9.80 (s, 1H), 8.28 (s, 1H), 7.71 (d, J = 8.5 Hz, 1H), 7.24 (s, 1H), 6.94 (d, J = 7.8 Hz, 1H), 6.26 (s, 1H), 5.00 (s, 1H), 3.61- 3.53 (m, 1H), 3.06 (q, J = 8.4 Hz, 1H), 2.50 (s, 1H), 2.03 (q, J = 9.5, 8.5 Hz, 1H), 1.92 (s, 1H), 1.74 (s, 3H), 1.62 (s, 6H), 1.41 (s, 6H), 1.24 (s, 2H), 1.16 (s, 6H), 1.06-0.97 (m, 1H). | 413.15 |
| rel-(1R,3S)-3-(5- ((7,7-dimethyl-5- oxo-6,7-dihydro- 5H-pyrrolo[3,4- b]pyridin-2- yl)amino)-1H- pyrazol-3- yl)cyclopentyl isopropylcarbamate | 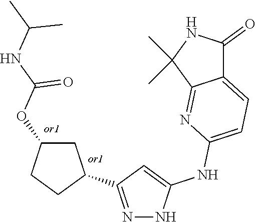 [SMILES: CC(C)NC(=O)O[C@H]1CC[C@@H](c2cc(Nc3ccc4c(n3)C(C)(C)NC4=O)[nH]n2)C1] | ¹H NMR (400 MHz, DMSO-d₆) 11.98 (s, 1H), 9.80 (s, 1H), 8.29 (s, 1H), 7.71 (d, J = 8.6 Hz, 1H), 7.25 (s, 1H), 6.95 (d, J = 7.9 Hz, 1H), 6.27 (s, 1H), 5.01 (s, 1H), 3.58 (d, J = 7.5 Hz, 1H), 3.07 (s, 1H), 2.50 (s, 1H), 2.18-1.84 (m, 2H), 1.73 (d, J = 8.9 Hz, 3H), 1.62 (s, 6H), 1.41 (s, 1H), 1.03 (d, J = 4.9 Hz, 6H). | 413.25 |
| rel-(1R,3S)-3-(5- ((7,7-dimethyl-5- oxo-6,7-dihydro- 5H-pyrrolo[3,4- b]pyridin-2- yl)amino)-1H- pyrazol-3- yl)cyclopentyl methylcarbamate | 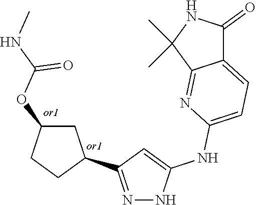 [SMILES: CNC(=O)O[C@@H]1CC[C@H](c2cc(Nc3ccc4c(n3)C(C)(C)NC4=O)[nH]n2)C1] | ¹H NMR (400 MHz, DMSO-d₆) 11.98 (s, 1H), 9.79 (s, 1H), 8.29 (s, 1H), 7.71 (d, J = 8.6 Hz, 1H), 7.26 (s, 1H), 6.92 (d, J = 4.9 Hz, 1H), 6.26 (s, 1H), 5.01 (s, 1H), 3.12- 3.03 (m, 1H), 2.55 (d, J = 4.6 Hz, 3H), 2.50-2.42 (m, 1H), 2.11-1.98 (m, 1H), 1.92 (s, 1H), 1.79-1.64 (m, 3H), 1.63 (s, 6H), 1.41 (s, 1H). | 385.10 |
| rel-(1R,3S)-3-(5- ((7,7-dimethyl-5- oxo-6,7-dihydro- 5H-pyrrolo[3,4- b]pyridin-2- yl)amino)-1H- pyrazol-3- yl)cyclopentyl methylcarbamate | 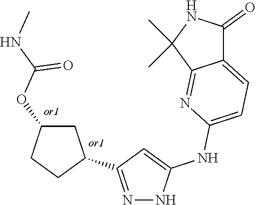 [SMILES: CNC(=O)O[C@H]1CC[C@@H](c2cc(Nc3ccc4c(n3)C(C)(C)NC4=O)[nH]n2)C1] | ¹H NMR (400 MHz, DMSO-d₆) 11.98 (s, 1H), 9.81 (s, 1H), 8.29 (s, 1H), 7.71 (d, J = 8.6 Hz, 1H), 7.24 (s, 1H), 6.92 (d, J = 4.5 Hz, 1H), 6.25 (s, 1H), 5.01 (s, 1H), 3.12- 3.03 (m, 1H), 2.55 (d, J = 4.6 Hz, 3H), 2.46 (s, 1H), 2.14-1.85 (m, 2H), 1.82-1.55 (m, 3H), 1.41 (s, 6H), 1.24 (s, 1H), 1.00 (t, J = 7.1 Hz, 1H).. | 385.10 |
| rel-(3R,5R)-5-(3- ((7,7-dimethyl-5- oxo-6,7-dihydro- 5H-pyrrolo[3,4- b]pyridin-2- yl)amino)-1H- pyrazol-5-yl) tetrahydrofuran-3-yl isopropylcarbamate | 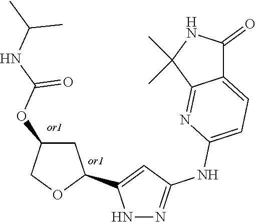 [SMILES: CC(C)NC(=O)O[C@@H]1CO[C@H](c2cc(Nc3ccc4c(n3)C(C)(C)NC4=O)n[nH]2)C1] | ¹H NMR (400 MHz, DMSO-d₆) 12.22 (s, 1H), 9.85 (s, 1H), 8.30 (s, 1H), 7.73 (d, J = 8.6 Hz, 1H), 7.28 (s, 1H), 7.09 (d, J = 7.8 Hz, 1H), 6.43 (s, 1H), 5.15 (m, 1H), 4.85 (s, 1H), 3.92 (s, 2H), 3.63 (m, 1H), 2.80 (s, 1H), 1.92 (s, 1H), 1.42 (s, 6H), 1.03 (dd, J = 6.7, 2.4 Hz, 6H). | 415.20 |
| rel-(3R,5R)-5-(3- ((7,7-dimethyl-5- oxo-6,7-dihydro- 5H-pyrrolo[3,4- b]pyridin-2- yl)amino)-1H- pyrazol-5-yl) tetrahydrofuran-3-yl isopropylcarbamate | 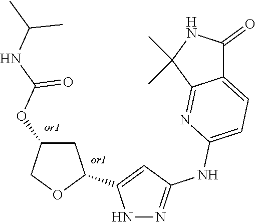 [SMILES: CC(C)NC(=O)O[C@H]1CO[C@@H](c2cc(Nc3ccc4c(n3)C(C)(C)NC4=O)n[nH]2)C1] | ¹H NMR (400 MHz, DMSO-d₆) 12.22 (s, 1H), 9.85 (s, 1H), 8.30 (s, 1H), 7.73 (d, J = 8.6 Hz, 1H), 7.26 (s, 1H), 7.09 (d, J = 7.8 Hz, 1H), 6.41 (s, 1H), 5.20 (m, 1H), 4.85 (s, 1H), 3.86 (s, 2H), 3.64 (m, 1H), 2.70 (s, 1H), 1.94 (s, 1H), 1.42 (s, 6H), 1.04 (dd, J = 6.7, 2.4 Hz, 6H). | 415.20 |
| rel-7,7-dimethyl-2- ((3-((1R,3S)-3- ((2-methylpyridin- 3-yl)oxy) cyclopentyl)- 1H-pyrazol-5- yl)amino)-6,7- dihydro-5H-pyrrolo [3,4-b]pyridin-5-one | 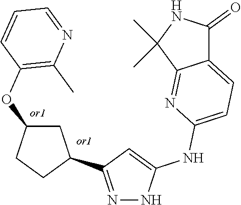 [SMILES: Cc1ncccc1O[C@@H]1CC[C@H](c2cc(Nc3ccc4c(n3)C(C)(C)NC4=O)[nH]n2)C1] | ¹H NMR (400 MHz, DMSO-d₆) 12.02 (s, 1H), 9.84 (s, 1H), 8.27 (s, 1H), 7.98 (dd, J = 4.8, 1.3 Hz, 1H), 7.69 (d, J = 8.5 Hz, 1H), 7.31 (dd, J = 8.4, 1.3 Hz, 1H), 7.16 (dd, J = 8.2, 4.7 Hz, 2H), 6.30 (s, 1H), 4.94 (dt, J = 6.5, 3.4 Hz, 1H), 3.25-3.12 (m, 1H), 2.79 (q, J = 7.2 Hz, 1H), 2.63 (ddd, J = 14.5, 8.9, 6.6 Hz, 1H), 2.31 (s, 3H), 2.16-2.03 (m, 1H), 2.03-1.90 (m, 1H), 1.90-1.73 (m, 3H), 1.36 (d, J = 7.2 Hz, 6H), 1.12 (t, J = 7.2 Hz, 2H). | 419.15 |
| rel-7,7-dimethyl-2- ((3-((1R,3S)-3- ((2-methylpyridin- 3-yl)oxy) cyclopentyl)- 1H-pyrazol-5- yl)amino)-6,7- dihydro-5H-pyrrolo [3,4-b]pyridin-5-one | 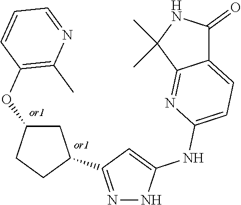 [SMILES: Cc1ncccc1O[C@H]1CC[C@@H](c2cc(Nc3ccc4c(n3)C(C)(C)NC4=O)[nH]n2)C1] | ¹H NMR (400 MHz, DMSO-d₆) 12.02 (s, 1H), 9.84 (s, 1H), 8.27 (s, 1H), 7.98 (dd, J = 4.8, 1.3 Hz, 1H), 7.69 (d, J = 8.5 Hz, 1H), 7.31 (dd, J = 8.4, 1.3 Hz, 1H), 7.16 (dd, J = 8.2, 4.7 Hz, 2H), 6.30 (s, 1H), 4.94 (dt, J = 6.5, 3.4 Hz, 1H), 3.25-3.12 (m, 1H), 2.79 (q, J = 7.2 Hz, 1H), 2.63 (ddd, J = 14.5, 8.9, 6.6 Hz, 1H), 2.31 (s, 3H), 2.16-2.03 (m, 1H), 2.03-1.90 (m, 1H), 1.90-1.73 (m, 3H), 1.36 (d, J = 7.2 Hz, 6H), 1.12 (t, J = 7.2 Hz, 1H). | 419.20 |

### Example 12

### (1R,3S)-3-(3-(isothiazol-3-ylamino)-1H-pyrazol-5-yl)cyclopentyl isopropylcarbamate and (1S,3R)-3-(3-(isothiazol-3-ylamino)-1H-pyrazol-5-yl)cyclopentyl isopropylcarbamate

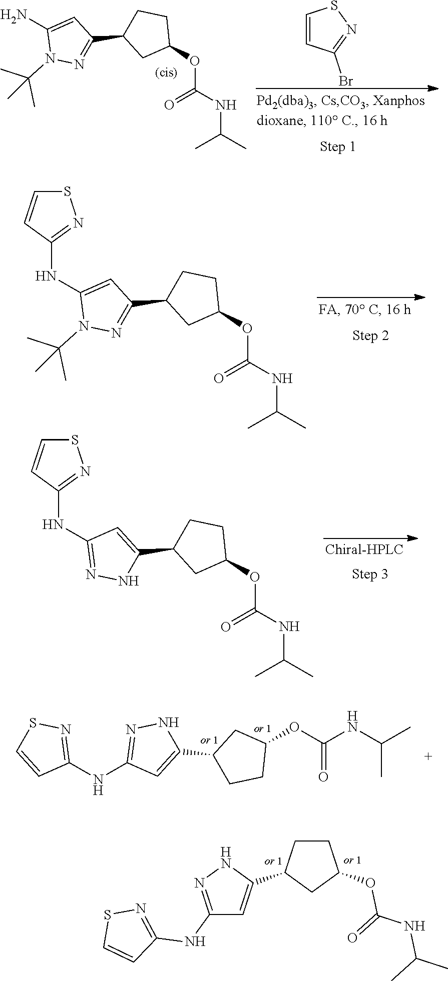 [SMILES: CC(C)NC(=O)O[C@@H]1CC[C@H](c2cc(N)n(C(C)(C)C)n2)C1 ; Brc1ccsn1 ; CC(C)NC(=O)O[C@@H]1CC[C@H](c2cc(Nc3ccsn3)n(C(C)(C)C)n2)C1 ; CC(C)NC(=O)O[C@@H]1CC[C@H](c2cc(Nc3ccsn3)n[nH]2)C1 ; CC(C)NC(=O)O[C@@H]1CC[C@H](c2cc(Nc3ccsn3)n[nH]2)C1 ; CC(C)NC(=O)O[C@H]1CC[C@@H](c2cc(Nc3ccsn3)n[nH]2)C1]

### cis-3-(1-(tert-butyl)-5-(isothiazol-3-ylamino)-1H-pyrazol-3-yl)cyclopentyl isopropylcarbamate

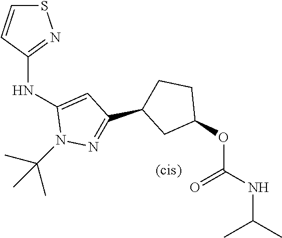 [SMILES: CC(C)NC(=O)O[C@@H]1CC[C@H](c2cc(Nc3ccsn3)n(C(C)(C)C)n2)C1]

Step 1: 3-bromoisothiazole (223 mg, 2 eq, 1.36 mmol), cesium carbonate (887 mg, 4 eq, 2.72 mmol) and (9,9-dimethyl-9H-xanthene-4,5-diyl) bis(diphenylphosphane) (39.4 mg, 0.1 eq, 68.1 μmol) were added to a solution of cis-3-(3-amino-1-(tert-butyl)-1H-pyrazol-5-yl) cyclopentyl isopropylcarbamate (210 mg, 1 Eq, 681 μmol) in dioxane (4 mL). After bubbling nitrogen through the reaction mixture for 5 minutes, Pd₂(dba)₃ (62.3 mg, 0.1 Eq, 68.1 μmol) was added. The reaction mixture is heated at 110° C. for 16 hours with vigorous stirring. After cooling to room temperature, the mixture was evaporated and extracted with ethyl acetate (3×40 mL), dried over Na₂SO₄ and evaporated in vacuo. The crude residue was purified by flash (Mobile Phase A: Water (10 mmol/L NH₄HCO₃), Mobile Phase B: ACN; Flow rate: 60 mL/min; Gradient: 45% B to 55% B in 6 min); After solvent evaporation afforded cis-3-(1-(tert-butyl)-3-(isothiazol-3-ylamino)-1H-pyrazol-5-yl) cyclopentylisopropylcarbamate (102 mg, 261 μmol, 38.3%) as a light yellow solid.

m/z (ES⁺) [M+H]⁺=392.50; HPLC tR=0.854 min.

### cis-3-(3-(isothiazol-3-ylamino)-1H-pyrazol-5-yl)cyclopentyl isopropylcarbamate

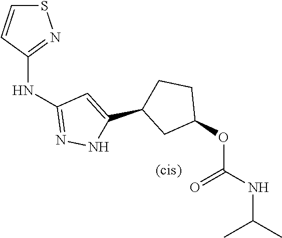 [SMILES: CC(C)NC(=O)O[C@@H]1CC[C@H](c2cc(Nc3ccsn3)n[nH]2)C1]

Step 2: The solution of cis-3-(1-(tert-butyl)-3-(isothiazol-3-ylamino)-1H-pyrazol-5-yl) cyclopentyl isopropyl carbamate (115 mg, 1 Eq, 294 μmol) in FA (2 mL) was heated at 70° C. for 16 h. After cooling to room temperature, the mixture was evaporated. The residue was purified by flash (Mobile Phase A: Water (10 mmol/L NH₄HCO₃), Mobile Phase B: ACN; Flow rate: 55 mL/min; Gradient: 45% B to 55% B in 7 min); After solvent evaporation afforded cis-3-(3-(isothiazol-3-ylamino)-1H-pyrazol-5-yl)cyclopentyl isopropyl carbamate (60 mg, 0.18 mmol, 61%) as a colorless amorphous solid.

m/z (ES⁺) [M+H]⁺=336.25; HPLC tR=0.921 min.

### (1R,3S)-3-(3-(isothiazol-3-ylamino)-1H-pyrazol-5-yl)cyclopentyl isopropylcarbamate and (1S,3R)-3-(3-(isothiazol-3-ylamino)-1H-pyrazol-5-yl)cyclopentyl isopropylcarbamate

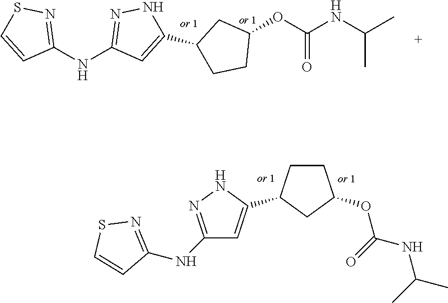 [SMILES: CC(C)NC(=O)O[C@@H]1CC[C@H](c2cc(Nc3ccsn3)n[nH]2)C1 ; CC(C)NC(=O)O[C@H]1CC[C@@H](c2cc(Nc3ccsn3)n[nH]2)C1]

Step 3: Cis-3-(3-(isothiazol-3-ylamino)-1H-pyrazol-5-yl) cyclopentyl isopropylcarbamate (60 mg, 1 eq, 0.18 mmol) was purified by Chiral-HPLC (Column: CHIRALPAK IE, 2*25 cm, 5 m; Mobile Phase A: Hex (0.2% DEA)—HPLC, Mobile Phase B: EtOH: DCM=1:1—HPLC; Flow rate: 20 mL/min; Gradient: 40% B to 40% B in 15 min; Wave Length: 220/254 nm; RT1(min): 8.13; RT2(min): 11.87; Sample Solvent: EtOH: DCM=1:1—HPLC; Injection Volume: 0.6 mL). Lyophilization yielded (1R,3S)-3-(3-(isothiazol-3-ylamino)-1H-pyrazol-5-yl)cyclopentyl isopropylcarbamate (18.6 mg, 55.5 μmol, 62%) as a white amorphous solid.

m/z (ES⁺) [M+H]⁺=336.05; HPLC tR=0.676 min.

¹H NMR (400 MHz, Chloroform-d) δ 8.49 (d, J=4.7 Hz, 1H), 7.51 (s, 1H), 6.87 (d, J=4.7 Hz, 1H), 6.27 (s, 1H), 5.22 (s, 1H), 4.69 (s, 1H), 3.83 (s, 1H), 3.22 (q, J=8.1 Hz, 1H), 2.50 (s, 1H), 2.14 (s, 1H), 1.93 (d, J=22.8 Hz, 4H), 1.17 (t, J=5.5 Hz, 6H).

Lyophilization yielded (1S,3R)-3-(3-(isothiazol-3-ylamino)-1H-pyrazol-5-yl)cyclopentyl isopropylcarbamate (14.9 mg, 44.4 μmol, 50%) as a white amorphous solid.

m/z (ES⁺) [M+H]⁺=336.05; HPLC tR=0.675 min.

¹H NMR (400 MHz, Chloroform-d) δ 8.49 (d, J=4.8 Hz, 1H), 7.58 (s, 1H), 6.88 (d, J=4.8 Hz, 1H), 6.29 (s, 1H), 5.22 (s, 1H), 4.71 (s, 1H), 3.83 (s, 1H), 3.21 (t, J=8.2 Hz, 1H), 2.50 (s, 1H), 2.15 (d, J=8.6 Hz, 1H), 1.91 (s, 4H), 1.17 (t, J=5.7 Hz, 6H).

### Example 13

### 1-isopropyl-3-((1R,3S)-3-(3-(pyrimidin-2-ylamino)-1H-pyrazol-5-yl)cyclopentyl)urea and 1-isopropyl-3-((1S,3R)-3-(3-(pyrimidin-2-ylamino)-1H-pyrazol-5-yl)cyclopentyl)urea

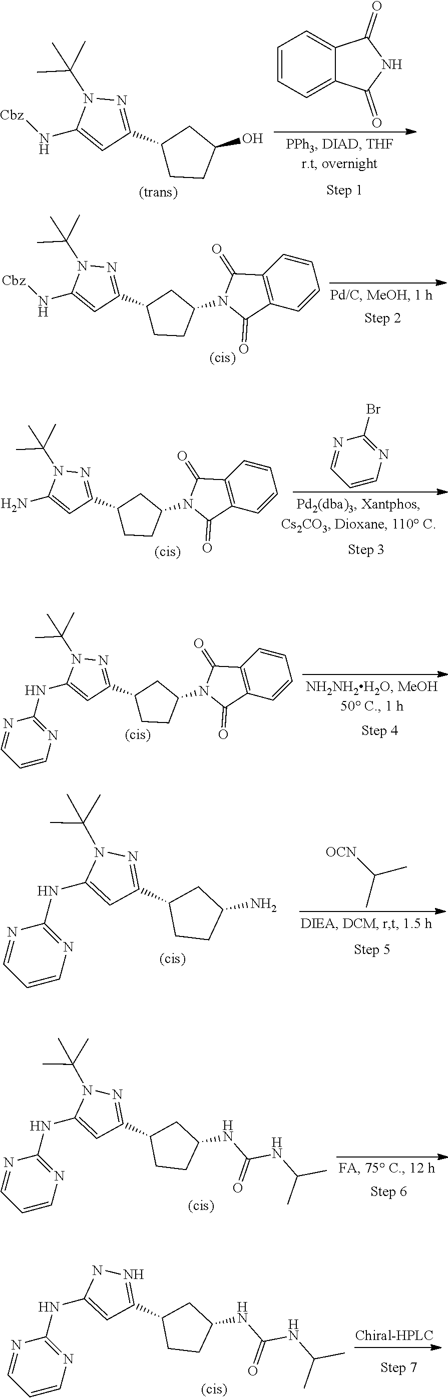

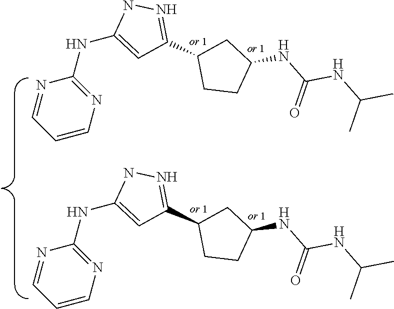 [SMILES: CC(C)NC(=O)N[C@@H]1CC[C@H](c2cc(Nc3ncccn3)[nH]n2)C1 ; CC(C)NC(=O)N[C@H]1CC[C@@H](c2cc(Nc3ncccn3)[nH]n2)C1]

### benzyl cis-(1-(tert-butyl)-3-(3-(1,3-dioxoisoindolin-2-yl)cyclopentyl)-1H-pyrazol-5-yl)carbamate

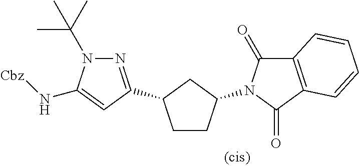 [SMILES: CC(C)(C)n1nc([C@H]2CC[C@@H](N3C(=O)c4ccccc4C3=O)C2)cc1NC(=O)OCc1ccccc1]

Step 1: To a mixture of benzyl cis-(1-(tert-butyl)-5-(3-hydroxycyclopentyl)-1H-pyrazol-3-yl)carbamate (780 mg, 2.18 mmol), PPh₃ (743 mg, 2.84 mmol) andisoindoline-1,3-dione (385 mg, 2.62 mmol) in THF (10 mL) was added DIAD (574 mg, 2.84 mmol) drop wise at 0° C. under nitrogen atmosphere. The mixture was stirred for 1 h at 0° C. After the mixture was stirred for 12 h at 25° C. The reaction mixture was diluted with H₂O (50 mL), and the aqueous phase was extracted with EA (50 mL) three times. The combined organic layers were washed with brine, dried over sodium sulfate, filtered, and concentrated in vacuo. The crude product was purified by silica gel chromatography (2 g column; eluting with PE/EA; ratio:8/1). Concentration in vacuo resulted in benzyl cis-(1-(tert-butyl)-5-(3-(1,3-dioxoisoindolin-2-yl)cyclopentyl)-1H-pyrazol-3-yl)carbamate (1 g, 90%) as a colorless oil.

m/z (ES⁺) [M+H]⁺=487.35; HPLC tR=1.285 min.

### cis-2-(3-(5-amino-1-(tert-butyl)-1H-pyrazol-3-yl)cyclopentyl)isoindoline-1,3-dione

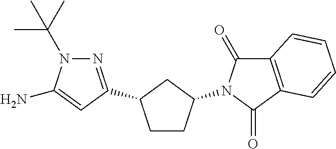 [SMILES: CC(C)(C)n1nc([C@H]2CC[C@@H](N3C(=O)c4ccccc4C3=O)C2)cc1N]

Step 2: A stirred mixture of cis-benzyl (1-(tert-butyl)-5-(3-(1,3-dioxoisoindolin-2-yl)cyclopentyl)-1H-pyrazol-3-yl)carbamate (1 g, 2 mmol) and Pd/C (0.2 g) in THF (10 mL) was treated with H₂ for 2 h at 25° C. The reaction mixture was filtered (through a pad of Celite), the pad was washed with EA, and the filtrate was concentrated in vacuo. Concentration in vacuo resulted in cis-2-(3-(3-amino-1-(tert-butyl)-1H-pyrazol-5-yl)cyclopentyl)isoindoline-1,3-dione (670 mg, 90%) as a colorless oil.

m/z (ES⁺) [M+H]⁺=0.868; HPLC tR=353.25 min.

### cis-2-(3-(1-(tert-butyl)-5-(pyrimidin-2-ylamino)-1H-pyrazol-3-yl)cyclopentyl)isoindoline-1,3-dione

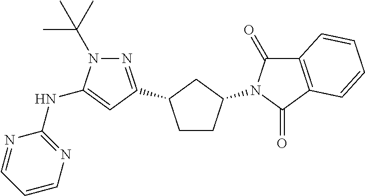 [SMILES: CC(C)(C)n1nc([C@H]2CC[C@@H](N3C(=O)c4ccccc4C3=O)C2)cc1Nc1ncccn1]

Step 3: A resealable reaction vial was charged with cis-2-(3-(3-amino-1-(tert-butyl)-1H-pyrazol-5-yl)cyclopentyl)isoindoline-1,3-dione (1.5 g, 4.3 mmol), 2-bromopyrimidine (0.81 g, 5.1 mmol),Cs₂CO₃ (4.2 g, 13 mmol),Pd₂(dba)₃ (0.39 g, 0.43 mmol), xantphos (0.49 g, 0.85 mmol), Dioxane (20 mL) was added, and a stirbar before being evacuated and purged with nitrogen three times, and the mixture was stirred for 1 h at 110° C. The reaction mixture was diluted with H₂O (50 mL), and the aqueous phase was extracted with EA (100 mL) three times. The combined organic layers were washed with brine, dried over sodium sulfate, filtered, and concentrated in vacuo. The crude product was purified by silica gel chromatography (10 g column; eluting with PE/EA; ratio:2/1). Concentration in vacuo resulted in cis-2-(3-(1-(tert-butyl)-3-(pyrimidin-2-ylamino)-1H-pyrazol-5-yl)cyclopentyl)isoindoline-1,3-dione (1 g, 50%) as an orange solid.

m/z (ES⁺) [M+H]⁺=431.25; HPLC tR=1.127 min.

### cis-N-(3-(3-aminocyclopentyl)-1-(tert-butyl)-1H-pyrazol-5-yl)pyrimidin-2-amine

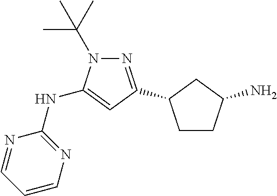 [SMILES: CC(C)(C)n1nc([C@H]2CC[C@@H](N)C2)cc1Nc1ncccn1]

Step 4: A resealable reaction vial was charged with cis-2-(3-(1-(tert-butyl)-3-(pyrimidin-2-ylamino)-1H-pyrazol-5-yl)cyclopentyl)isoindoline-1,3-dione (1 g, 2 mmol) NH₂NH₂·H₂O/MeOH (3:1) (4 mL) was added, and a stirbar before being evacuated and purged with nitrogen three times. and the mixture was stirred for 1 h at 50° C. The residue was purified by reverse flash chromatography with the following conditions: column, C18 silica gel; mobile phase, MeCN in water, 10% to 50% gradient in 20 min; detector, UV 250 nm. Concentration in vacuo resulted in cis-N-(5-(3-aminocyclopentyl)-1-(tert-butyl)-1H-pyrazol-3-yl)pyrimidin-2-amine (500 mg, 70%) as a colourless solid.

m/z (ES⁺) [M+H]⁺=301.30; HPLC tR=0.718 min.

### cis-1-(3-(1-(tert-butyl)-5-(pyrimidin-2-ylamino)-1H-pyrazol-3-yl)cyclopentyl)-3-isopropylurea

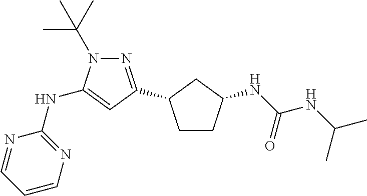 [SMILES: CC(C)NC(=O)N[C@@H]1CC[C@H](c2cc(Nc3ncccn3)n(C(C)(C)C)n2)C1]

Step 5: To a mixture of cis-N-(5-(3-aminocyclopentyl)-1-(tert-butyl)-1H-pyrazol-3-yl)pyrimidin-2-amine (560 mg, 1.86 mmol) and DIEA (723 mg, 5.59 mmol) in DCM (5 mL) was added 2-isocyanatopropane (190 mg, 2.24 mmol) drop wise at 25° C. under nitrogen atmosphere. The mixture was stirred for 2 h at 25° C. The residue was purified by reverse flash chromatography with the following conditions: column, C18 silica gel; mobile phase, MeCN in water, 10% to 50% gradient in 15 min; detector, UV 220 nm. Concentration in vacuo resulted in cis-1-(3-(1-(tert-butyl)-3-(pyrimidin-2-ylamino)-1H-pyrazol-5-yl)cyclopentyl)-3-isopropylurea (360 mg, 50.1%) as a white solid.

m/z (ES⁺) [M+H]⁺=386.40; HPLC tR=0.902 min.

### cis-1-isopropyl-3-(3-(3-(pyrimidin-2-ylamino)-1H-pyrazol-5-yl)cyclopentyl)urea

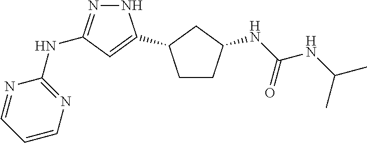 [SMILES: CC(C)NC(=O)N[C@@H]1CC[C@H](c2cc(Nc3ncccn3)n[nH]2)C1]

Step 6: A resealable reaction vial was charged with cis-1-(3-(1-(tert-butyl)-3-(pyrimidin-2-ylamino)-1H-pyrazol-5-yl)cyclopentyl)-3-isopropylurea (340 mg, 882 μmol), FA (10 mL) was added, and a stirbar before being evacuated and purged with nitrogen three times, and the mixture was stirred for 12 h at 75° C. The reaction was concentrated under vacuum. The reaction mixture was poured into 10 mL MeOH. The isolated solid was collected, The solid resulted in cis-1-isopropyl-3-(3-(3-(pyrimidin-2-ylamino)-1H-pyrazol-5-yl)cyclopentyl)urea (220 mg, 75.7%) as a white solid.

m/z (ES⁺) [M+H]⁺=330.30; HPLC tR=0.735 min.

### 1-isopropyl-3-((1R,3S)-3-(3-(pyrimidin-2-ylamino)-1H-pyrazol-5-yl)cyclopentyl)urea

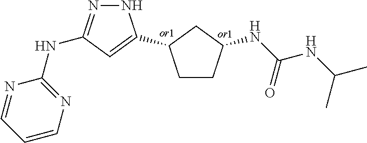 [SMILES: CC(C)NC(=O)N[C@@H]1CC[C@H](c2cc(Nc3ncccn3)n[nH]2)C1]

Step 7: Cis-1-isopropyl-3-(3-(3-(pyrimidin-2-ylamino)-1H-pyrazol-5-yl)cyclopentyl)urea (220 mg, 668 μmol) material was purified by chiral Pre-HPLC (Column: CHIRALPAK IE, 2*25 cm, 5 m; Mobile Phase A: Hex(0.2% CH₃COOH)—HPLC, Mobile Phase B: EtOH: DCM=1:1—HPLC; Flow rate: 20 mL/min; Gradient: 40% B to 40% B in 31 min; Wave Length: 220/254 nm; RT1(min): 19.81; Sample Solvent: EtOH: DCM=1:1—HPLC; Injection Volume: 1.2 mL; Number Of Runs: 14). Lyophilization resulted in 1-isopropyl-3-((1R,3S)-3-(3-(pyrimidin-2-ylamino)-1H-pyrazol-5-yl)cyclopentyl)urea (64 mg, 0.19 mmol, 29%) as a white solid.

m/z (ES⁺) [M+H]⁺=330.20; HPLC tR=0.615 min.

¹H NMR (400 MHz, DMSO-d₆) δ 11.93 (s, 1H), 9.55 (s, 1H), 8.42 (d, J=4.8 Hz, 2H), 6.76 (t, J=4.8 Hz, 1H), 6.32 (s, 1H), 5.82 (d, J=7.6 Hz, 1H), 5.54 (d, J=7.7 Hz, 1H), 3.96 (p, J=7.4 Hz, 1H), 3.65 (dp, J=7.7, 6.4 Hz, 1H), 3.08-2.95 (m, 1H), 2.31 (dt, J=13.6, 7.3 Hz, 1H), 1.94 (dddd, J=33.7, 15.2, 12.3, 7.4 Hz, 2H), 1.68 (dtd, J=10.8, 8.8, 8.2, 6.4 Hz, 1H), 1.51-1.32 (m, 2H), 1.02 (dd, J=6.5, 1.0 Hz, 6H).

### 1-isopropyl-3-((1S,3R)-3-(3-(pyrimidin-2-ylamino)-1H-pyrazol-5-yl)cyclopentyl)urea

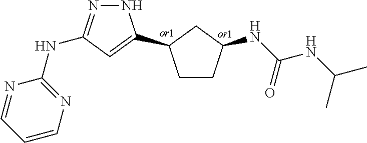 [SMILES: CC(C)NC(=O)N[C@H]1CC[C@@H](c2cc(Nc3ncccn3)n[nH]2)C1]

Cis-1-isopropyl-3-(3-(3-(pyrimidin-2-ylamino)-1H-pyrazol-5-yl)cyclopentyl)urea (220 mg, 668 μmol) material was purified by chiral Pre-HPLC (Column: CHIRALPAK IE, 2*25 cm, m; Mobile Phase A: Hex(0.2% CH₃COOH)—HPLC, Mobile Phase B: EtOH: DCM=1:1—HPLC; Flow rate: 20 mL/min; Gradient: 40% B to 40% B in 31 min; Wave Length: 220/254 nm; RT2(min): 25.25; Sample Solvent: EtOH: DCM=1:1—HPLC; Injection Volume: 1.2 mL; Number Of Runs: 14). Lyophilization resulted in 1-isopropyl-3-((1S,3R)-3-(3-(pyrimidin-2-ylamino)-1H-pyrazol-5-yl)cyclopentyl)urea (65.7 mg, 29.9%) as a white solid.

m/z (ES⁺) [M+H]⁺=330.15; HPLC tR=0.615 min.

¹H NMR (400 MHz, DMSO-d₆) δ 11.92 (s, 1H), 9.55 (s, 1H), 8.42 (d, J=4.7 Hz, 2H), 6.76 (t, J=4.8 Hz, 1H), 6.32 (s, 1H), 5.82 (d, J=7.5 Hz, 1H), 5.54 (d, J=7.7 Hz, 1H), 3.96 (h, J=7.4 Hz, 1H), 3.72-3.59 (m, 1H), 3.00 (h, J=8.6 Hz, 1H), 2.31 (dt, J=13.4, 7.1 Hz, 1H), 2.04-1.83 (m, 2H), 1.68 (dtd, J=10.9, 8.8, 8.1, 6.4 Hz, 1H), 1.51-1.32 (m, 2H), 1.02 (dd, J=6.5, 1.0 Hz, 6H).

Additional compounds prepared according to the methods of Example 13 are depicted in Table 8 below.

TABLE 8
Additional Exemplary Compounds

| | | | MS |
|---|---|---|---|
| Compound | Structure | Proton NMR | [M + 1] |
| N-(5-((1S,3R)-3-(3- isopropylureido) cyclopentyl)- 1H-pyrazol- 3-yl)-2-(3- methylisoxazol-5- yl)acetamide | 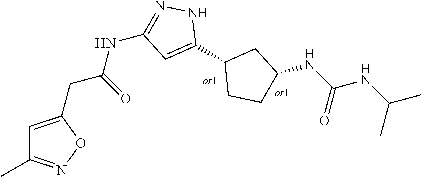 [SMILES: Cc1cc(CC(=O)Nc2cc([C@H]3CC[C@@H](NC(=O)NC(C)C)C3)[nH]n2)on1] | ¹H NMR (400 MHz, DMSO-d₆) 12.10 (s, 1H), 10.62 (s, 1H), 6.29 (s, 1H), 6.22 (s, 1H), 5.78 (d, J = 7.6 Hz, 1H), 5.53 (d, J = 7.7 Hz, 1H), 3.96 (dt, J = 15.1, 7.5 Hz, 1H), 3.83 (s, 2H), 3.64 (dq, J = 13.4, 6.4 Hz, 1H), 3.01 (p, J = 8.4 Hz, 1H), 2.30 (dt, J = 13.5, 7.2 Hz, 1H), 2.20 (s, 3H), 2.02-1.83 (m, 2H), 1.70-1.57 (m, 1H), 1.48-1.39 (m, 1H), 1.37-1.27 (m, 1H), 1.01 (d, J = 6.5 Hz, 6H) | 375.35 |
| N-(5-((1R,3S)-3-(3- isopropylureido) cyclopentyl)- 1H-pyrazol- 3-yl)-2-(3- methylisoxazol-5- yl)acetamide | 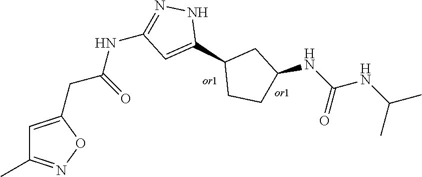 [SMILES: Cc1cc(CC(=O)Nc2cc([C@@H]3CC[C@H](NC(=O)NC(C)C)C3)[nH]n2)on1] | ¹H NMR (400 MHz, DMSO-d₆) 12.10 (s, 1H), 10.63 (s, 1H), 6.26 (d, J = 26.7 Hz, 2H), 5.78 (d, J = 7.6 Hz, 1H), 5.53 (d, J = 7.7 Hz, 1H), 3.95 (h, J = 7.4 Hz, 1H), 3.83 (s, 2H), 3.71-3.58 (m, 1H), 3.00 (p, J = 8.2 Hz, 1H), 2.29 (dd, J = 13.0, 6.6 Hz, 1H), 2.20 (s, 3H), 2.15-1.74 (m, 2H), 1.70-1.57 (m, 1H), 1.43 (dt, J = 11.9, 6.2 Hz, 1H), 1.38-1.22 (m, 1H), 1.01 (d, J = 6.5 Hz, 6H) | 375.30 |
| N-(5-((1S,3R)- 3-(3-isopropyl- 1-methylureido) cyclopentyl)- 1H-pyrazol- 3-yl)-2-(3- methylisoxazol-5- yl)acetamide | 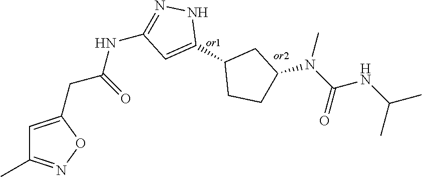 [SMILES: Cc1cc(CC(=O)Nc2cc([C@H]3CC[C@@H](N(C)C(=O)NC(C)C)C3)[nH]n2)on1] | ¹H NMR (400 MHz, DMSO-d₆) 12.15 (s, 1H), 10.65 (s, 1H), 6.32 (s, 1H), 6.23 (s, 1H), 5.86 (d, J = 7.6 Hz, 1H), 4.65 (d, J = 8.5 Hz, 1H), 3.84 (s, 2H), 3.76 (h, J = 6.8 Hz, 1H), 3.00 (q, J = 8.3 Hz, 1H), 2.67 (s, 3H), 2.21 (s, 3H), 1.99 (dt, J = 11.9, 6.6 Hz, 2H), 1.79-1.52 (m, 4H), 1.06 (d, J = 6.6 Hz, 6H). | 389.15 |
| N-(5-((1R,3S)- 3-(3-isopropyl- 1-methylureido) cyclopentyl)- 1H-pyrazol- 3-yl)-2-(3- methylisoxazol-5- yl)acetamide | 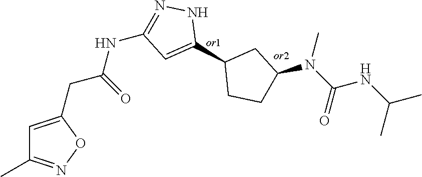 [SMILES: Cc1cc(CC(=O)Nc2cc([C@@H]3CC[C@H](N(C)C(=O)NC(C)C)C3)[nH]n2)on1] | ¹H NMR (400 MHz, DMSO-d₆) 12.15 (s, 1H), 10.65 (s, 1H), 6.31 (s, 1H), 6.23 (s, 1H), 5.86 (d, J = 7.6 Hz, 1H), 4.66 (p, J = 7.8 Hz, 1H), 3.84 (s, 2H), 3.76 (dq, J = 13.5, 6.7 Hz, 1H), 3.06-2.94 (m, 1H), 2.67 (s, 3H), 2.21 (s, 3H), 2.00 (dd, J = 12.4, 6.6 Hz, 2H), 1.80-1.52 (m, 4H), 1.06 (d, J = 6.6 Hz, 6H). | 389.20 |
| N-(5-((1S,3S)- 3-(3-isopropyl- 1-methylureido) cyclopentyl)- 1H-pyrazol- 3-yl)-2-(3- methylisoxazol-5- yl)acetamide | 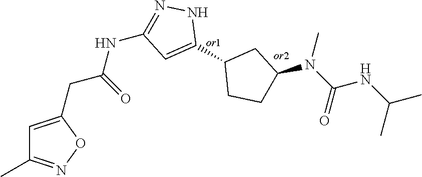 [SMILES: Cc1cc(CC(=O)Nc2cc([C@H]3CC[C@H](N(C)C(=O)NC(C)C)C3)[nH]n2)on1] | ¹H NMR (400 MHz, DMSO-d₆) 12.09 (s, 1H), 10.61 (s, 1H), 6.29 (s, 1H), 6.23 (s, 1H), 5.82 (d, J = 7.6 Hz, 1H), 4.76-4.67 (m, 1H), 3.83 (s, 2H), 3.75 (dq, J = 13.4, 6.7 Hz, 1H), 3.20 (t, J = 8.0 Hz, 1H), 2.67 (s, 3H), 2.20 (s, 3H), 2.05 (s, 1H), 1.94-1.71 (m, 3H), 1.56 (dq, J = 11.8, 6.0 Hz, 2H), 1.05 (d, J = 6.6 Hz, 6H) | 389.20 |
| N-(5-((1R,3R)- 3-(3-isopropyl- 1-methylureido) cyclopentyl)- 1H-pyrazol- 3-yl)-2-(3- methylisoxazol-5- yl)acetamide | 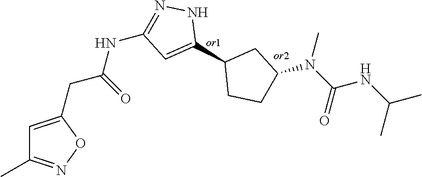 [SMILES: Cc1cc(CC(=O)Nc2cc([C@@H]3CC[C@@H](N(C)C(=O)NC(C)C)C3)[nH]n2)on1] | ¹H NMR (400 MHz, DMSO-d₆) 12.12 (s, 1H), 10.62 (s, 1H), 6.29 (s, 1H), 6.23 (s, 1H), 5.83 (d, J = 7.9 Hz, 1H), 4.72 (p, J = 8.3 Hz, 1H), 3.83 (s, 2H), 3.81-3.68 (m, 1H), 3.20 (p, J = 8.2 Hz, 1H), 2.67 (s, 3H), 2.20 (s, 3H), 2.05 (s, 1H), 1.94-1.71 (m, 3H), 1.56 (tq, J = 10.2, 5.2 Hz, 2H), 1.05 (d, J = 6.5 Hz, 6H) | 389.20 |

### Example 14

### N-((1R,3S)-3-(3-(2-(3-methylisoxazol-5-yl)acetamido)-1H-pyrazol-5-yl)cyclopentyl)benzamide and N-((1S,3R)-3-(3-(2-(3-methylisoxazol-5-yl)acetamido)-1H-pyrazol-5-yl)cyclopentyl)benzamide

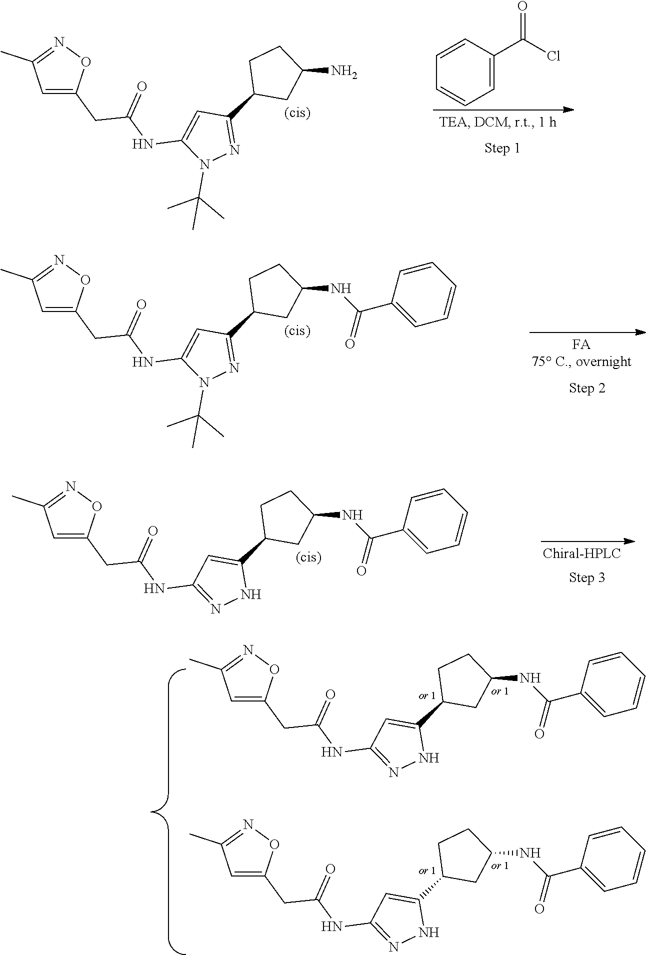 [SMILES: Cc1cc(CC(=O)Nc2cc([C@H]3CC[C@@H](N)C3)nn2C(C)(C)C)on1 ; O=C(Cl)c1ccccc1 ; Cc1cc(CC(=O)Nc2cc([C@H]3CC[C@@H](NC(=O)c4ccccc4)C3)nn2C(C)(C)C)on1 ; Cc1cc(CC(=O)Nc2cc([C@H]3CC[C@@H](NC(=O)c4ccccc4)C3)[nH]n2)on1 ; Cc1cc(CC(=O)Nc2cc([C@H]3CC[C@@H](NC(=O)c4ccccc4)C3)[nH]n2)on1 ; Cc1cc(CC(=O)Nc2cc([C@@H]3CC[C@H](NC(=O)c4ccccc4)C3)[nH]n2)on1]

### cis-N-(3-(1-(tert-butyl)-5-(2-(3-methylisoxazol-5-yl)acetamido)-1H-pyrazol-3-yl)cyclopentyl)benzamide

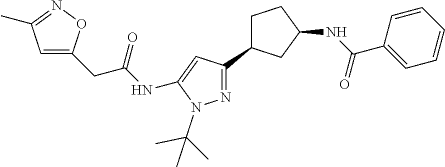 [SMILES: Cc1cc(CC(=O)Nc2cc([C@H]3CC[C@@H](NC(=O)c4ccccc4)C3)nn2C(C)(C)C)on1]

Step 1: A round bottomed flask was charged with N-(5-(cis-3-aminocyclopentyl)-1-(tert-butyl)-1H-pyrazol-3-yl)-2-(3-methylisoxazol-5-yl)acetamide (180 mg, 1 Eq, 374 μmol), DCM (6 mL), TEA (120 mg, 3 Eq, 1.12 mmol) and a stirbar. Benzoyl chloride (63.1 mg, 1.2 Eq, 449 μmol) was added at 0° C., and the solution was stirred at 25° C. for 1 hour. The resulting crude material was purified by TLC(DCM/MeOH=10:1). Concentration in vacuo resulted in N-(cis-3-(1-(tert-butyl)-3-(2-(3-methylisoxazol-5-yl)acetamido)-1H-pyrazol-5-yl)cyclopentyl)benzamide (130 mg, 289 μmol, 77.3%) as a yellow amorphous solid.

m/z (ES⁺) [M+H]⁺=450.15; HPLC tR=0.942 min.

### cis-N-(3-(3-(2-(3-methylisoxazol-5-yl)acetamido)-1H-pyrazol-5-yl)cyclopentyl)benzamide

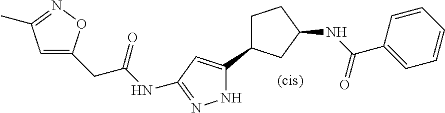 [SMILES: Cc1cc(CC(=O)Nc2cc([C@H]3CC[C@@H](NC(=O)c4ccccc4)C3)[nH]n2)on1]

Step 2: A round bottomed flask was charged with N-(cis-3-(1-(tert-butyl)-3-(2-(3-methylisoxazol-5-yl)acetamido)-1H-pyrazol-5-yl)cyclopentyl)benzamide (130 mg, 1 Eq, 289 μmol), FA (3 mL) was added, and the solution was stirred at 75° C. for 16 hour. The resulting crude material was purified by Flash chromatography (acetonitrile/water). Lyophilization yielded cis-N-(3-(3-(2-(3-methylisoxazol-5-yl)acetamido)-1H-pyrazol-5-yl)cyclopentyl)benzamide (90 mg, 0.23 mmol, 79%) as a white amorphous solid.

m/z (ES⁺) [M+H]⁺=394.10; HPLC tR=0.517 min.

### N-((1R,3S)-3-(3-(2-(3-methylisoxazol-5-yl)acetamido)-1H-pyrazol-5-yl)cyclopentyl)benzamide and N-((1S,3R)-3-(3-(2-(3-methylisoxazol-5-yl)acetamido)-1H-pyrazol-5-yl)cyclopentyl)benzamide

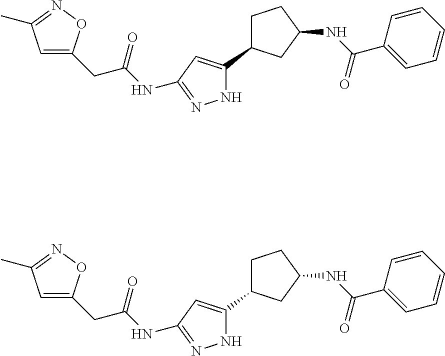 [SMILES: Cc1cc(CC(=O)Nc2cc([C@H]3CC[C@@H](NC(=O)c4ccccc4)C3)[nH]n2)on1 ; Cc1cc(CC(=O)Nc2cc([C@@H]3CC[C@H](NC(=O)c4ccccc4)C3)[nH]n2)on1]

Step 3: N-(cis-3-(3-(2-(3-methylisoxazol-5-yl)acetamido)-1H-pyrazol-5-yl)cyclopentyl)benzamide (90 mg, 1 Eq, 0.23 mmol) was purified by Chiral-HPLC(Column: CHIRALPAK IG, 2*25 cm, 5 m; Mobile Phase A: Hex(0.2% DEA)—HPLC, Mobile Phase B: EtOH: DCM=1:1—HPLC; Flow rate: 20 mL/min; Gradient: 60% B to 60% B in 11 min; Wave Length: 220/254 nm; RT1(min): 6.97; RT2 (min): 8.65; Sample Solvent: EtOH: DCM=1:1—HPLC; Injection Volume: 0.3 mL; Number Of Runs: 6). Lyophilization yielded N-((1R,3S)-3-(3-(2-(3-methylisoxazol-5-yl)acetamido)-1H-pyrazol-5-yl)cyclopentyl)benzamide (27.5 mg, 0.070 mmol, 30.5%) as a white amorphous solid and N-((1S,3R)-3-(3-(2-(3-methylisoxazol-5-yl)acetamido)-1H-pyrazol-5-yl)cyclopentyl)benzamide (29.6 mg, 0.075 mmol, 32.9%) as a white amorphous solid.

### N-((1R,3S)-3-(3-(2-(3-methylisoxazol-5-yl)acetamido)-1H-pyrazol-5-yl)cyclopentyl)benzamide

m/z (ES⁺) [M+H]⁺=394.15; HPLC tR=1.128 min.

¹H NMR (400 MHz, DMSO-d₆) 12.13 (s, 1H), 10.64 (s, 1H), 8.42 (d, J=7.6 Hz, 1H), 7.88-7.81 (m, 2H), 7.56-7.48 (m, 1H), 7.48-7.41 (m, 2H), 6.35 (s, 1H), 6.22 (s, 1H), 4.37 (q, J=7.6 Hz, 1H), 3.83 (s, 2H), 3.13-3.02 (m, 1H), 2.39 (dd, J=12.9, 7.0 Hz, 1H), 2.20 (s, 3H), 2.02 (t, J=7.9 Hz, 2H), 1.78-1.69 (m, 2H), 1.69-1.61 (m, 1H).

### N-((1S,3R)-3-(3-(2-(3-methylisoxazol-5-yl)acetamido)-1H-pyrazol-5-yl)cyclopentyl)benzamide

m/z (ES⁺) [M+H]⁺=394.20; HPLC tR=1.112 min.

¹H NMR (400 MHz, DMSO-d₆) 12.13 (s, 1H), 10.64 (s, 1H), 8.42 (d, J=7.4 Hz, 1H), 7.87-7.81 (m, 2H), 7.56-7.49 (m, 1H), 7.49-7.41 (m, 2H), 6.35 (s, 1H), 6.22 (s, 1H), 4.42-4.34 (m, 1H), 3.83 (s, 2H), 3.11-3.02 (m, 1H), 2.39 (dd, J=13.1, 7.1 Hz, 2H), 2.20 (s, 3H), 2.02 (t, J=8.0 Hz, 2H), 1.81-1.61 (m, 4H), 1.08 (t, J=7.2 Hz, 1H).

### Example 15

### 2-(3-methylisoxazol-5-yl)-N-(5-(thieno[3,2-d]pyrimidin-7-yl)-1H-pyrazol-3-yl)acetamide

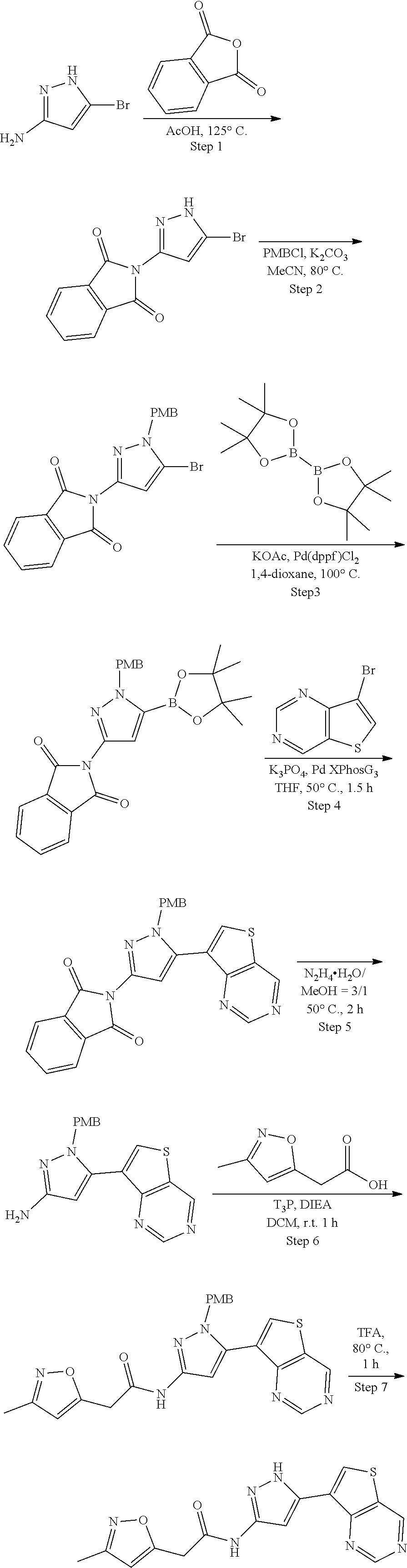

### 2-(5-bromo-1H-pyrazol-3-yl)isoindoline-1,3-dione

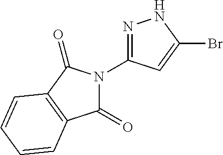 [SMILES: O=C1c2ccccc2C(=O)N1c1cc(Br)[nH]n1]

Step 1: To a solution of 5-bromo-1H-pyrazol-3-amine (2.5 g, 1 eq, 15 mmol) and isobenzofuran-1,3-dione (2.7 g, 1.2 eq, 19 mmol) in AcOH (25 mL) at room temperature under nitrogen atmosphere. The reaction mixture was stirred for 16 h at 125° C. The mixture was cooled to r.t. The mixture was concentrated and the residue was diluted with water and extracted with ethyl acetate. The combined organic layer was washed with brine, dried over sodium sulfate and concentrated under reduced pressure to afford 2-(5-bromo-1H-pyrazol-3-yl)isoindoline-1,3-dione (4.4 g, 15 mmol, 98%) as a yellow solid.

m/z (ES⁺) [M+H]⁺=291.85; HPLC tR=0.773 min.

### 2-(5-bromo-1-(4-methoxybenzyl)-1H-pyrazol-3-yl)isoindoline-1,3-dione

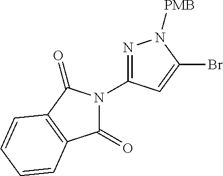

Step 2: To a solution of 2-(5-bromo-1H-pyrazol-3-yl)isoindoline-1,3-dione (4 g, 0.01 mol) and K₂CO₃ (6 g, 0.04 mol) in MeCN (30 mL) was added 1-(chloromethyl)-4-methoxybenzene (3 g, 0.02 mol) at room temperature under nitrogen atmosphere. The reaction mixture was stirred for 16 h at 80° C. The mixture was cooled to r.t, The resulting mixture was filtered, the filter cake was washed with EA (3×250 mL). The filtrate was concentrated under reduced pressure. The residue was purified by silica gel column chromatography, eluting with PE/EA (2/1) to afford 2-(5-bromo-1-(4-methoxybenzyl)-1H-pyrazol-3-yl)isoindoline-1,3-dione (5 g, 0.01 mol, 90%) as an off-white solid.

m/z (ES⁺) [M+H]⁺=411.95; HPLC tR=0.939 min.

### 2-(1-(4-methoxybenzyl)-5-(4,4,5,5-tetramethyl-1,3,2-dioxaborolan-2-yl)-1H-pyrazol-3-yl)isoindoline-1,3-dione

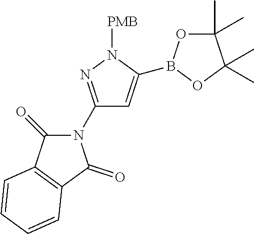

Step 3: To a solution of 2-(5-bromo-1-(4-methoxybenzyl)-1H-pyrazol-3-yl)isoindoline-1,3-dione (5 g, 0.01 mol) and 4,4,4′,4′,5,5,5′,5′-octamethyl-2,2′-bi(1,3,2-dioxaborolane) (6 g, 0.02 mol) in 1,4-Dioxane (20 mL) was added potassium acetate (4 g, 0.04 mol) and PdCl₂(dppf)-CH2Cl2 adduct (1 g, 1 mmol) at room temperature under nitrogen atmosphere. The reaction mixture was stirred for 16 h at 100° C. The mixture was cooled to r.t. The resulting mixture was filtered, the filter cake was washed with EA (3×250 mL). The filtrate was concentrated under reduced pressure. The residue was purified by silica gel column chromatography, eluted with PE/EA (1/1) to afford 2-(1-(4-methoxybenzyl)-5-(4,4,5,5-tetramethyl-1,3,2-dioxaborolan-2-yl)-1H-pyrazol-3-yl)isoindoline-1,3-dione (2.6 g, 4.5 mmol, 40%, 80% purity) as an off-white solid.

m/z (ES⁺) [M+H]⁺=460.05; HPLC tR=1.225 min.

### 2-(1-(4-methoxybenzyl)-5-(thieno[3,2-d]pyrimidin-7-yl)-1H-pyrazol-3-yl)isoindoline-1,3-dione

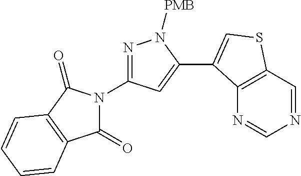

Step 4: To a solution of 2-(1-(4-methoxybenzyl)-5-(4,4,5,5-tetramethyl-1,3,2-dioxaborolan-2-yl)-1H-pyrazol-3-yl) isoindoline-1,3-dione (600 mg, 1.31 mmol) and 7-bromothieno[3,2-d] pyrimidine (281 mg, 1.31 mmol) was dissolved in THE (10 mL), K₃PO4 (831 mg, 3.92 mmol) and Pd XantPhos G3 (111 mg, 131 μmol) were added. The resulting mixture was stirred for 1.5 h at 50° C. under N₂ atmosphere. Desired product could be detected by LCMS. The resulting mixture was concentrated under vacuum. The residue was purified by reverse flash chromatography with the following conditions: column, C18 silica gel; mobile phase, ACN in water, 0% to 100% gradient in 20 min; detector, UV 254 nm. This resulted in 2-(1-(4-methoxybenzyl)-5-(thieno[3,2-d]pyrimidin-7-yl)-1H-pyrazol-3-yl)isoindoline-1,3-dione (190 mg, 406 μmol, 31.1%) as a yellow solid.

m/z (ES⁺) [M+H]⁺=468.00; HPLC tR=1.052 min.

### 1-(4-methoxybenzyl)-5-(thieno[3,2-d]pyrimidin-7-yl)-1H-pyrazol-3-amine

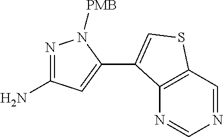

Step 5: To a solution of 2-(1-(4-methoxybenzyl)-5-(thieno[3,2-d]pyrimidin-7-yl)-1H-pyrazol-3-yl)isoindoline-1,3-dione (190 mg, 406 μmol) was dissolved in MeOH (2 mL) and N₂H₄·H₂O (6 mL). The resulting mixture was stirred for 2 hour at 50° C. under nitrogen atmosphere. The resulting mixture was concentrated under vacuum. Desired product could be detected by LCMS. The residue was purified by reverse flash chromatography with the following conditions: Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient: 30% B to 50% B in 8 min, detector, UV 254 nm. This resulted in 1-(4-methoxybenzyl)-5-(thieno[3,2-d]pyrimidin-7-yl)-1H-pyrazol-3-amine (100 mg, 296 μmol, 72.9%) as an orange solid.

m/z (ES⁺) [M+H]⁺=338.00; HPLC tR=0.734 min.

### N-(1-(4-methoxybenzyl)-5-(thieno[3,2-d]pyrimidin-7-yl)-1H-pyrazol-3-yl)-2-(3-methylisoxazol-5-yl)acetamide

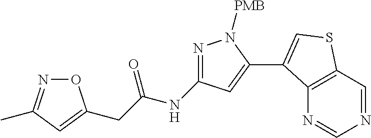

Step 6: To a stirred solution of 1-(4-methoxybenzyl)-5-(thieno[3,2-d]pyrimidin-7-yl)-1H-pyrazol-3-amine (100 mg, 296 μmol),2-(3-methylisoxazol-5-yl)acetic acid (62.7 mg, 445 μmol) and DIEA (115 mg, 889 μmol) in DCM (3 mL) were added T3P (283 mg, 889 μmol) dropwise at r.t. The resulting mixture was stirred for 1 hour at 25° C. under nitrogen atmosphere. The mixture was diluted with water and extracted with ethyl acetate. The combined organic layer was washed with brine, dried over sodium sulfate and concentrated under reduced pressure. The mixture was concentrated to afford N-(1-(4-methoxybenzyl)-5-(thieno[3,2-d]pyrimidin-7-yl)-1H-pyrazol-3-yl)-2-(3-methylisoxazol-5-yl)acetamide (120 mg, 261 μmol, 87.9%) as a yellow solid.

m/z (ES⁺) [M+H]⁺=461.00; HPLC tR=0.911 min.

### 2-(3-methylisoxazol-5-yl)-N-(5-(thieno[3,2-d]pyrimidin-7-yl)-1H-pyrazol-3-yl)acetamide

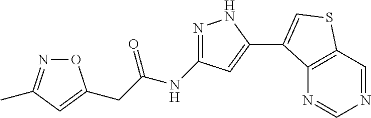 [SMILES: Cc1cc(CC(=O)Nc2cc(-c3csc4cncnc34)[nH]n2)on1]

Step 7: Into a vial was added N-(1-(4-methoxybenzyl)-5-(thieno[3,2-d]pyrimidin-7-yl)-1H-pyrazol-3-yl)-2-(3-methylisoxazol-5-yl)acetamide (110 mg, 239 μmol) and TFA (2 mL) at room temperature. The resulting mixture was stirred for additional 80° C. at 1 hour. The resulting mixture was concentrated under vacuum. The crude residue was purified by Prep-HPLC (Column: XBridge Prep OBD C18 Column, 30*150 mm, 5 m; Mobile Phase A: Water(10 mmol/L NH₄HCO₃), Mobile Phase B: ACN; Flow rate: 60 mL/min; Gradient: 10% B to 35% B in 8 min, 35% B; Wave Length: 220 nm). This resulted in 2-(3-methylisoxazol-5-yl)-N-(5-(thieno[3,2-d]pyrimidin-7-yl)-1H-pyrazol-3-yl)acetamide (42.6 mg, 125 μmol, 52.4%) as a white solid.

m/z (ES⁺) [M+H]⁺=341.05; HPLC tR=0.975 min.

¹H NMR (400 MHz, DMSO-d₆) 13.03 (s, 1H), 10.89 (s, 1H), 9.63 (s, 1H), 9.31 (s, 1H), 8.77 (s, 1H), 7.48 (s, 1H), 6.28 (s, 1H), 3.91 (s, 2H), 2.22 (s, 3H).

### Example 16

### N-(5-(bicyclo[2.2.1]heptan-1-yl)-1H-pyrazol-3-yl)pyrimidin-2-amine

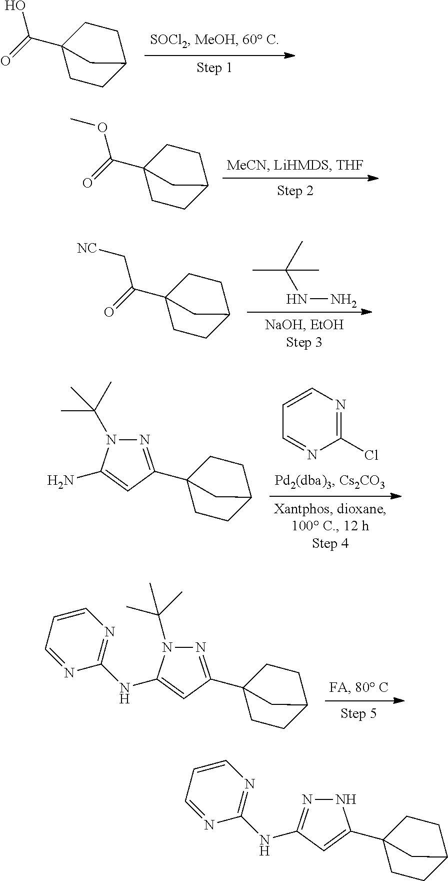

### methyl bicyclo[2.2.1]heptane-1-carboxylate

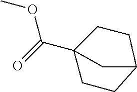

Step 1: Sulfurous dichloride (1.31 g, 2.2 eq, 11.0 mmol) was dropwise added to a solution of bicyclo[2.2.1]heptane-1-carboxylic acid (700 mg, 1 eq, 4.99 mmol) in MeOH (16 mL) at 0° C. under nitrogen atmosphere. The mixture was stirred for 3 h at 60° C. The solvent was removed under reduced pressure at room temperature to obtain methyl bicyclo[2.2.1]heptane-1-carboxylate (720 mg, 4.67 mmol, 93.5%) as a white solid. The crude product was used for the next step without further purification.

m/z (ES⁺) [M+H]⁺=155.10; HPLC tR=1.035 min.

### 3-(bicyclo[2.2.1]heptan-1-yl)-3-oxopropanenitrile

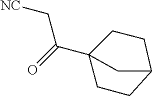 [SMILES: N#CCC(=O)C12CCC(CC1)C2]

Step 2: LiHMDS (1.17 g, 7.00 mL, 1 molar, 1.5 eq, 7.00 mmol) was added to the solution of methyl bicyclo[2.2.1]heptane-1-carboxylate (720 mg, 1 eq, 4.67 mmol) and acetonitrile (383 mg, 2 eq, 9.34 mmol) in THE (16 mL) at −78° C. The reaction mixture was stirred at −78° C. for 1 hour. LCMS showed no SM. The reaction mixture was quenched with saturated NH₄Cl (15 mL), and the aqueous phase was extracted with DCM (60 mL) three times. The combined organic layers were washed with brine, dried over sodium sulfate, filtered, and concentrated in vacuo to obtain 3-(bicyclo[2.2.1]heptan-1-yl)-3-oxopropanenitrile (730 mg, 4.47 mmol, 95.8%) which was used for the next step without further purification.

m/z (ES⁺) [M+H]⁺=no mass signal; HPLC tR=0.976 min.

### 3-(bicyclo[2.2.1]heptan-1-yl)-1-(tert-butyl)-1H-pyrazol-5-amine

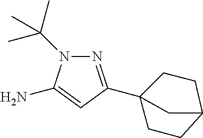 [SMILES: CC(C)(C)n1nc(C23CCC(CC2)C3)cc1N]

Step 3: tert-butylhydrazine hydrochloride (836 mg, 1.5 eq, 6.71 mmol) and sodium hydroxide (179 mg, 1 eq, 4.47 mmol) were stirred in EtOH (5 mL) for 1 h. The solution was added dropwise to a solution of 3-(bicyclo[2.2.1]heptan-1-yl)-3-oxopropanenitrile (730 mg, 1 Eq, 4.47 mmol) in EtOH (8 mL). The solution was stirred at 50° C. for 2 hours. The reaction mixture was diluted with saturated NH₄Cl (20 mL), and the aqueous phase was extracted with DCM (40 mL) three times. The combined organic layers were washed with brine, dried over sodium sulfate, filtered, and concentrated in vacuo. The resulting crude material was purified by Flash (acetonitrile/water/0.1% formic acid). Concentration in vacuo to afford 5-(bicyclo[2.2.1]heptan-1-yl)-1-(tert-butyl)-1H-pyrazol-3-amine (600 mg, 2.57 mmol, 57.5%) as a brown solid.

m/z (ES⁺) [M+H]⁺=234.10; HPLC tR=0.824 min.

### N-(3-(bicyclo[2.2.1]heptan-1-yl)-1-(tert-butyl)-1H-pyrazol-5-yl)pyrimidin-2-amine

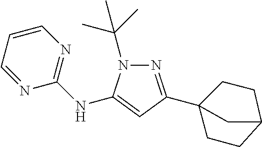 [SMILES: CC(C)(C)n1nc(C23CCC(CC2)C3)cc1Nc1ncccn1]

Step 4: Pd₂(dba)₃ (31 mg, 0.1 eq, 34 μmol) was added to the solution of xantphos (40 mg, 0.2 eq, 69 μmol), 5-(bicyclo[2.2.1]heptan-1-yl)-1-(tert-butyl)-1H-pyrazol-3-amine (80 mg, 1 eq, 0.34 mmol) and 2-chloropyrimidine (35 mg, 0.9 Eq, 0.31 mmol) in 1,4-dioxane (2 mL) under N₂. The reaction mixture was stirred at 80° C. for 16 hours under N₂ atmosphere. The reaction mixture was used in the next step without any work up.

m/z (ES⁺) [M+H]⁺=312.10; HPLC tR=1.114 min.

### N-(5-(bicyclo[2.2.1]heptan-1-yl)-1H-pyrazol-3-yl)pyrimidin-2-amine

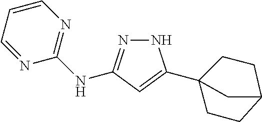 [SMILES: c1cnc(Nc2cc(C34CCC(CC3)C4)[nH]n2)nc1]

Step 5: To the reaction mixture from the previous step was added formic acid (975 mg, 60 Eq, 21.2 mmol) and the reaction was then heated to 80° C. for 16 hours. The reaction was concentrated to remove excess formic acid and directly purified by Flash (acetonitrile/water/0.1% formic acid). Concentration in vacuo afforded the crude product as a red oil. The crude product was further purified by Prep-HPLC with the following condition: Column: XBridge Shield R^{P}18 OBD Column, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 30% B to 50% B in 8 min, 50% B; Wave Length: 254 nm; RT1(min): 7.32. After solvent evaporation afforded N-(5-(bicyclo[2.2.1]heptan-1-yl)-1H-pyrazol-3-yl)pyrimidin-2-amine (36.5 mg, 143 μmol, 40.5%) as a white solid.

m/z (ES⁺) [M+H]⁺=256.15; HPLC tR=1.361 min.

¹H NMR (400 MHz, DMSO-d₆) δ 11.92 (s, 1H), 9.49 (s, 1H), 8.41 (d, J=4.8 Hz, 2H), 6.75 (t, J=4.8 Hz, 1H), 6.38 (s, 1H), 2.37-2.22 (m, 1H), 1.83-1.65 (m, 4H), 1.64-1.47 (m, 4H), 1.41-1.26 (m, 2H).

Additional compounds prepared according to the methods of Example 18 are set forth below:

### N-(5-(bicyclo[2.2.1]heptan-1-yl)-1H-pyrazol-3-yl)-2-(3-methylisoxazol-5-yl)acetamide

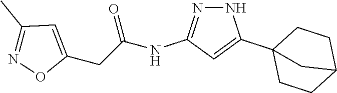 [SMILES: Cc1cc(CC(=O)Nc2cc(C34CCC(CC3)C4)[nH]n2)on1]

¹H NMR (400 MHz, DMSO-d₆) 12.13 (s, 1H), 10.63 (s, 1H), 6.29 (d, J=2.1 Hz, 1H), 6.22 (s, 1H), 3.83 (s, 2H), 2.30 (s, 1H), 2.20 (s, 3H), 1.77 ″C 1.63 (m, 4H), 1.61 ″C 1.47 (m, 4H), 1.40 ″C 1.28 (m, 2H).

m/z (ES⁺) [M+H]⁺=301.0.

### Example 17

### N-(3-((1s,3s)-3-(((4-cyclopropylisothiazol-3-yl)oxy)methyl)cyclobutyl)-1H-pyrazol-5-yl)-2-(3-methylisoxazol-5-yl)acetamide and N-(5-((1r,3r)-3-(((4-cyclopropylisothiazol-3-yl)oxy)methyl)cyclobutyl)-1H-pyrazol-3-yl)-2-(3-methylisoxazol-5-yl)acetamide

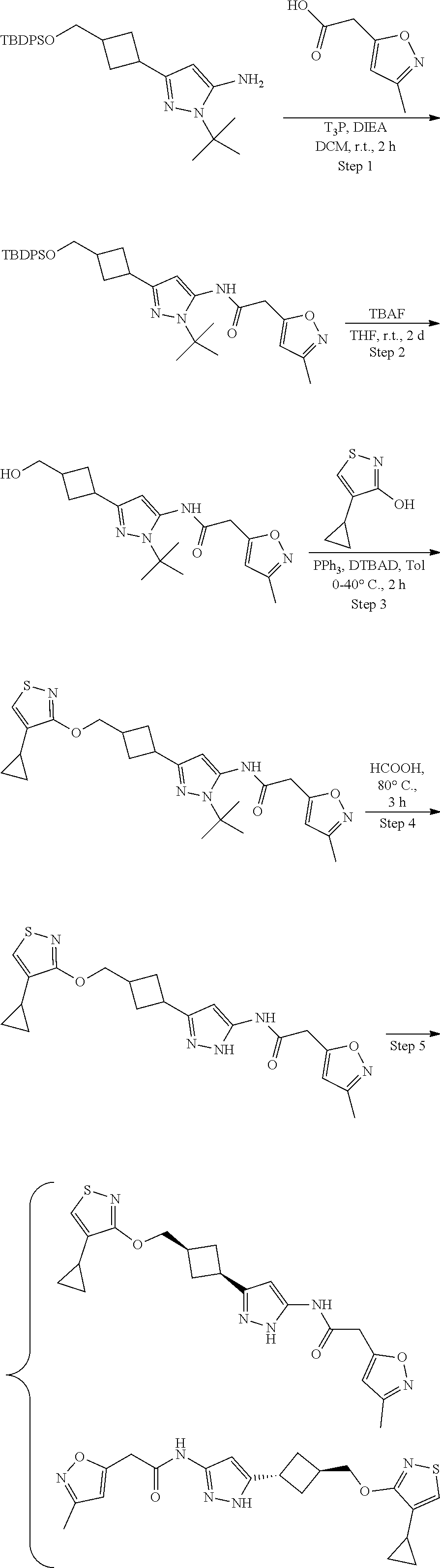

### N-(1-(tert-butyl)-3-(3-(((tert-butyldiphenylsilyl)oxy)methyl)cyclobutyl)-1H-pyrazol-5-yl)-2-(3-methylisoxazol-5-yl)acetamide

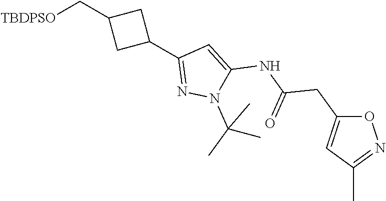

Step 1: A round bottomed flask was charged with 1-(tert-butyl)-5-(3-(((tert-butyldiphenylsilyl)oxy)methyl)cyclobutyl)-1H-pyrazol-3-amine (1.98 g, 1 eq, 4.29 mmol), EA (10 mL) and a stirbar, 2-(3-methylisoxazol-5-yl)acetic acid (908 mg, 1.5 eq, 6.43 mmol), DIEA (1.66 g, 3 eq, 12.9 mmol), T3P (5.45 g, 50% Wt, 2 eq, 8.58 mmol) was added, and the solution was stirred at 25° C. for 2 hour. The solution was quenched with water and the organic phase was collected. The aqueous phase was extracted with EA three times. The organic phase was combined and concentrated. The resulting crude material was purified by Flash (acetonitrile/water). Concentration in vacuo to afford N-(1-(tert-butyl)-5-(3-(((tert-butyldiphenylsilyl)oxy)methyl)cyclobutyl)-1H-pyrazol-3-yl)-2-(3-methylisoxazol-5-yl)acetamide (2.50 g, 4.27 mmol, 99.7%) as a brown amorphous solid.

### N-(1-(tert-butyl)-3-(3-(hydroxymethyl)cyclobutyl)-1H-pyrazol-5-yl)-2-(3-methylisoxazol-5-yl)acetamide

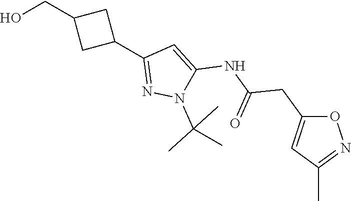 [SMILES: Cc1cc(CC(=O)Nc2cc(C3CC(CO)C3)nn2C(C)(C)C)on1]

Step 2: A round bottomed flask was charged with N-(1-(tert-butyl)-5-(3-(((tert-butyldiphenylsilyl)oxy)methyl)cyclobutyl)-1H-pyrazol-3-yl)-2-(3-methylisoxazol-5-yl)acetamide (2.5 g, 1 eq, 4.3 mmol), THE (15 mL) and a stirbar. TBAF (2.2 g, 8.5 mL, 1 molar, 2 eq, 8.5 mmol) was added, and the solution was stirred at 25° C. for 2 day. The mixture was diluted with water, and the aqueous phase was extracted with EA three times. The combined organic layers were washed with brine, dried over sodium sulfate, filtered, and concentrated in vacuo. The resulting crude material was purified by Flash (NH₃·OH₂O/water). Concentration in vacuo to afford N-(1-(tert-butyl)-5-(3-(hydroxymethyl)cyclobutyl)-1H-pyrazol-3-yl)-2-(3-methylisoxazol-5-yl)acetamide (900 mg, 2.60 mmol, 61%) as a brown amorphous solid.

### N-(1-(tert-butyl)-3-(3-(((4-cyclopropylisothiazol-3-yl)oxy)methyl)cyclobutyl)-1H-pyrazol-5-yl)-2-(3-methylisoxazol-5-yl)acetamide

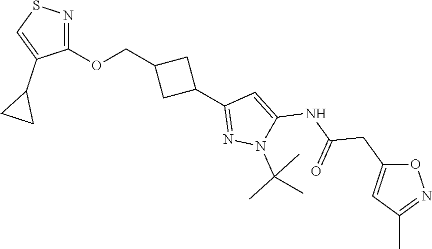 [SMILES: Cc1cc(CC(=O)Nc2cc(C3CC(COc4nscc4C4CC4)C3)nn2C(C)(C)C)on1]

Step 3: To a solution of N-(1-(tert-butyl)-5-(3-(hydroxymethyl)cyclobutyl)-1H-pyrazol-3-yl)-2-(3-methylisoxazol-5-yl)acetamide (150 mg, 1 eq, 433 μmol) in Toluene (5 mL), 4-cyclopropylisothiazol-3-ol (91.7 mg, 1.5 eq, 649 μmol) and triphenylphosphane (170 mg, 1.5 eq, 649 μmol) were added. After bubbling nitrogen through the reaction mixture for 1 minutes, di-tert-butyl (E)-diazene-1,2-dicarboxylate (150 mg, 1.5 Eq, 649 μmol) dropwised at 0° C. under nitrogen atmosphere. The mixture was stirred at 40° C. for 2 hours. The resulted solution was purified using C18 flash chromatography with the following conditions (Mobile Phase A: Water, Mobile Phase B: ACN; Flow rate: 60 mL/min; Gradient: 0% B to 100% B in 40 min; 254/220 nm). This resulted in N-(1-(tert-butyl)-5-(3-(((4-cyclopropylisothiazol-3-yl)oxy)methyl)cyclobutyl)-1H-pyrazol-3-yl)-2-(3-methylisoxazol-5-yl)acetamide (150 mg, 0.27 mmol, 62%, 84% Purity).

### N-(3-(3-(((4-cyclopropylisothiazol-3-yl)oxy)methyl)cyclobutyl)-1H-pyrazol-5-yl)-2-(3-methylisoxazol-5-yl)acetamide

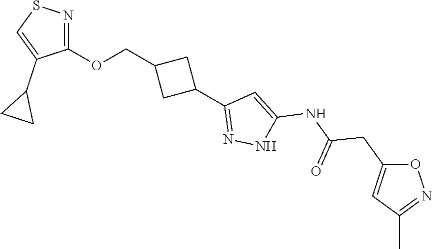 [SMILES: Cc1cc(CC(=O)Nc2cc(C3CC(COc4nscc4C4CC4)C3)n[nH]2)on1]

Step 4: The solution of N-(1-(tert-butyl)-5-(3-(((4-cyclopropylisothiazol-3-yl)oxy)methyl)cyclobutyl)-1H-pyrazol-3-yl)-2-(3-methylisoxazol-5-yl)acetamide (133 mg, 1 eq, 283 μmol) in FA (3 mL) was heated under 80° C. for 3 hour. The solvent was evaporated. The residue was purified by Prep-HPLC (Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient: 30% B to 55% B in 8 min, 55% B; Wave Length: 220 nm; RT1(min): 7.62. This resulted in N-(5-(3-(((4-cyclopropylisothiazol-3-yl)oxy)methyl)cyclobutyl)-1H-pyrazol-3-yl)-2-(3-methylisoxazol-5-yl)acetamide (100 mg, 242 μmol, 85.4%) as a white amorphous solid.

### N-(3-((1s,3s)-3-(((4-cyclopropylisothiazol-3-yl)oxy)methyl)cyclobutyl)-1H-pyrazol-5-yl)-2-(3-methylisoxazol-5-yl)acetamide

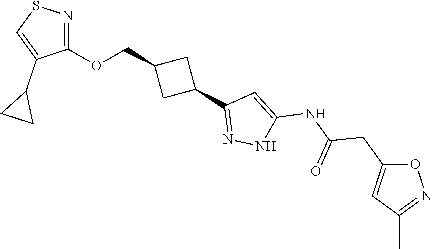 [SMILES: Cc1cc(CC(=O)Nc2cc([C@H]3C[C@@H](COc4nscc4C4CC4)C3)n[nH]2)on1]

Step 5: N-(3-((1s,3s)-3-(((4-cyclopropylisothiazol-3-yl)oxy)methyl)cyclobutyl)-1H-pyrazol-5-yl)-2-(3-methylisoxazol-5-yl)acetamide was purified by Chiral-HPLC.

¹H NMR (400 MHz, CDCl₃,) 0.6-0.73 (m, 2H), 0.86-1 (m, 2H), 1.82 (tt, J=8.4, 5.1 Hz, 1H), 2.18 (qd, J=9.5, 2.7 Hz, 2H), 2.32 (s, 3H), 2.51-2.63 (m, 2H), 3.49 (p, J=9.0 Hz, 1H), 3.89 (s, 2H), 4.40 (d, J=5.7 Hz, 2H), 6.18 (s, 1H), 6.56 (s, 1H), 7.84 (d, J=0.7 Hz, 1H), 8.82 (s, 1H).

m/z (ES⁺) [M+H]⁺=414.15.

### N-(5-((1r,3r)-3-(((4-cyclopropylisothiazol-3-yl)oxy)methyl)cyclobutyl)-1H-pyrazol-3-yl)-2-(3-methylisoxazol-5-yl)acetamide

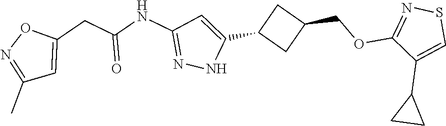 [SMILES: Cc1cc(CC(=O)Nc2cc([C@H]3C[C@H](COc4nscc4C4CC4)C3)[nH]n2)on1]

N-(3-((1r,3r)-3-(((4-cyclopropylisothiazol-3-yl)oxy)methyl)cyclobutyl)-1H-pyrazol-5-yl)-2-(3-methylisoxazol-5-yl)acetamide was purified by Chiral-HPLC.

### Example 18

Additional compounds set forth in Table 9 were synthesized according to the procedures described herein.

TABLE 9
Additional Exemplary Compounds

| | | | MS |
|---|---|---|---|
| | | | [M + |
| Compound | Structure | Proton NMR | 1] |
| 3-methyl-N- ((1R,3S)-3-(3- (2-(3-methyl- isoxazol- 5-yl)acetamido)- | 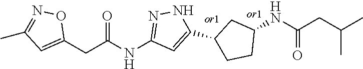 [SMILES: Cc1cc(CC(=O)Nc2cc([C@H]3CC[C@@H](NC(=O)CC(C)C)C3)[nH]n2)on1] | ¹H NMR (400 MHz, DMSO-d₆) 12.10 (s, 1H), 10.62 (s, 1H), 7.84 (d, J = 7.4 Hz, 1H), 6.31 (s, 1H), 6.22 (s, 1H), 4.11 (h, J = 7.6 Hz, 1H), 3.83 (s, 2H), 3.01 (p, J = 8.7 Hz, 1H), 2.30 (dd, J = 13.0, 6.8 | 374 |
| 1H-pyrazol-5- | | Hz, 1H), 2.20 (s, 3H), 2.03-1.93 (m, 2H), | |
| yl)cyclopentyl) | | 1.93-1.83 (m, 3H), 1.73-1.59 (m, 1H), | |
| butanamide | | 1.52 (t, J = 7.9 Hz, 1H), 1.49-1.36 (m, | |
| | | 1H), 0.89-0.83 (m, 6H). | |
| 3-methyl-N- ((1S,3R)-3-(3- (2-(3-methyl- isoxazol- 5-yl)acetamido)- | 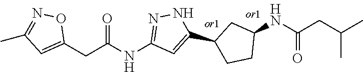 [SMILES: Cc1cc(CC(=O)Nc2cc([C@@H]3CC[C@H](NC(=O)CC(C)C)C3)[nH]n2)on1] | ¹H NMR (400 MHz, DMSO-d₆) 12.10 (s, 1H), 10.62 (s, 1H), 7.84 (d, J = 7.4 Hz, 1H), 6.31 (s, 1H), 6.22 (s, 1H), 4.11 (p, J = 7.5 Hz, 1H), 3.83 (s, 2H), 3.01 (p, J = 8.4 Hz, 1H), 2.32 (dq, J = 13.4, 7.1 | 374 |
| 1H-pyrazol-5- | | Hz, 1H), 2.20 (s, 3H), 2.03-1.83 (m, 5H), | |
| yl)cyclopentyl) | | 1.73-1.59 (m, 1H), 1.52 (t, J = 7.8 Hz, | |
| butanamide | | 1H), 1.43 (dt, J = 21.3, 10.1 Hz, 1H), | |
| | | 0.89-0.83 (m, 6H). | |
| N-(5-((1S,3R)- 3-(ethyl- sulfonamido) cyclopentyl)- 1H-pyrazol- 3-yl)-2-(3- methylisoxazol- 5-yl)acetamide | 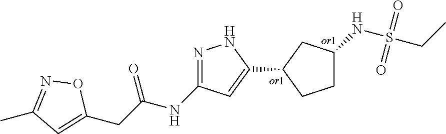 [SMILES: CCS(=O)(=O)N[C@@H]1CC[C@H](c2cc(NC(=O)Cc3cc(C)no3)n[nH]2)C1] | ¹H NMR (400 MHz, DMSO-d₆) 12.10 (s, 1H), 10.63 (s, 1H), 7.23 (d, J = 7.8 Hz, 1H), 6.33-6.28 (m, 1H), 6.22 (s, 1H), 3.83 (s, 2H), 3.70 (p, J = 7.8 Hz, 1H), 2.99 (q, J = 7.4 Hz, 3H), 2.35 (dt, J = 13.3, 7.1 Hz, 1H), 2.21 (s, 3H), 1.97 (dt, J = 11.0, 3.9 Hz, 2H), 1.71-1.56 (m, 2H), 1.51 (dt, J = 11.6, 9.9 Hz, 1H), 1.20 (t, J = 7.3 Hz, 3H). | 382 |
| N-(5-((1R,3S)- 3-(ethyl- sulfonamido) cyclopentyl)- 1H-pyrazol- 3-yl)-2-(3- methylisoxazol- 5-yl)acetamide | 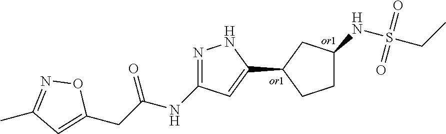 [SMILES: CCS(=O)(=O)N[C@H]1CC[C@@H](c2cc(NC(=O)Cc3cc(C)no3)n[nH]2)C1] | ¹H NMR (400 MHz, DMSO-d₆) 12.10 (s, 1H), 10.63 (s, 1H), 7.23 (d, J = 7.9 Hz, 1H), 6.31 (s, 1H), 6.22 (s, 1H), 3.83 (s, 2H), 3.69 (q, J = 7.7 Hz, 1H), 2.99 (q, J = 7.4 Hz, 3H), 2.41-2.29 (m, 1H), 2.21 (s, 3H), 2.00-1.93 (m, 2H), 1.71-1.45 (m, 3H), 1.20 (t, J = 7.3 Hz, 3H). | 382 |
| isopropyl ((1R,3S)- 3-(3-(2-(3- methylisoxazol- 5-yl)acetamido)- 1H-pyrazol-5- | 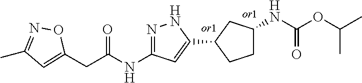 [SMILES: Cc1cc(CC(=O)Nc2cc([C@H]3CC[C@@H](NC(=O)OC(C)C)C3)[nH]n2)on1] | ¹H NMR (400 MHz, DMSO-d₆) 12.09 (s, 1H), 10.62 (s, 1H), 7.17 (d, J = 7.4 Hz, 1H), 6.30 (s, 1H), 6.23 (s, 1H), 4.74 (p, J = 6.3 Hz, 1H), 3.89 (d, J = 7.5 Hz, 1H), 3.83 (s, 2H), 2.99 (q, J = 8.5 Hz, 1H), 2.29 (dt, J = 13.9, 7.2 Hz, 1H), 2.21 | 376 |
| yl)cyclopentyl) | | (s, 3H), 1.93 (d, J = 8.2 Hz, 1H), 1.72- | |
| carbamate | | 1.61 (m, 1H), 1.57-1.49 (m, 1H), 1.49- | |
| | | 1.39 (m, 1H), 1.17 (d, J = 6.2 Hz, 6H). | |
| isopropyl ((1S,3R)- 3-(3-(2-(3- methylisoxazol- 5-yl)acetamido)- 1H-pyrazol-5- | 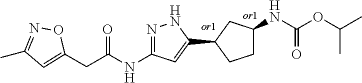 [SMILES: Cc1cc(CC(=O)Nc2cc([C@@H]3CC[C@H](NC(=O)OC(C)C)C3)[nH]n2)on1] | ¹H NMR (400 MHz, DMSO-d₆) 12.09 (s, 1H), 10.63 (s, 1H), 7.17 (d, J = 7.4 Hz, 1H), 6.30 (s, 1H), 6.23 (s, 1H), 4.74 (p, J = 6.2 Hz, 1H), 3.89 (q, J = 7.4 Hz, 1H), 3.83 (s, 2H), 3.00 (p, J = 8.4 Hz, 1H), 2.29 (dt, J = 13.7, 7.2 Hz, 1H), 2.21 | 376 |
| yl)cyclopentyl) | | (s, 3H), 2.01-1.82 (m, 2H), 1.72-1.60 (m, | |
| carbamate | | 1H), 1.54 (q, J = 6.9 Hz, 1H), 1.50-1.39 | |
| | | (m, 1H), 1.20-1.10 (m, 6H). | |
| 2-(3- methylisoxazol- 5-yl)-N- (5-((1S,3R)- 3-((N-methyl- sulfamoyl) amino) cyclopentyl)- 1H-pyrazol-3- | 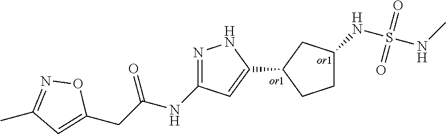 | ¹H NMR (400 MHz, DMSO-d₆) 11.83 (s, 1H), 10.74 (s, 1H), 7.03 (d, J = 171.0 Hz, 2H), 6.27 (s, 1H), 6.22 (s, 1H), 3.82 (s, 2H), 3.54 (t, J = 7.5 Hz, 1H), 2.99 (d, J = 8.2 Hz, 1H), 2.45 (s, 3H), 2.34 (td, J = 12.5, 11.6, 6.0 Hz, 1H), 2.20 (s, 3H), 1.93 (q, J = 7.2 Hz, 2H), 1.71-1.60 (m, 2H), 1.51 (td, J = 11.8, 9.2 Hz, 1H). | 383 |
| yl)acetamide | | | |
| 2-(3- methylisoxazol- 5-yl)-N-(5- ((1R,3S)-3-((N- methyl- sulfamoyl) amino) cyclopentyl)- 1H-pyrazol-3- | 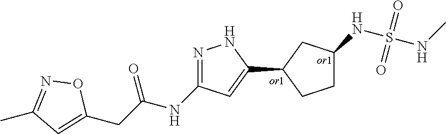 | ¹H NMR (400 MHz, DMSO-d₆) 11.92 (s, 1H), 10.64 (s, 1H), 7.01 (s, 1H), 6.67 (s, 1H), 6.28 (s, 1H), 6.22 (s, 1H), 3.82 (s, 2H), 3.55 (q, J = 7.6 Hz, 1H), 2.97 (d, J = 8.4 Hz, 1H), 2.45 (s, 3H), 2.35 (dq, J = 12.4, 6.4, 5.7 Hz, 1H), 2.20 (s, 3H), 1.93 (q, J = 7.5 Hz, 2H), 1.64 (q, J = 5.7 Hz, 2H), 1.57-1.44 (m, 1H) | 383 |
| yl)acetamide | | | |
| N-(5-(2-(iso- propylamino) thieno[3,2- d]pyrimidin- 7-yl)- 1H-pyrazol- 3-yl)-2-(3- methylisoxazol- 5-yl)acetamide | 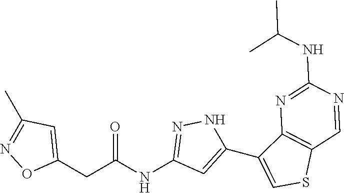 [SMILES: Cc1cc(CC(=O)Nc2cc(-c3csc4cnc(NC(C)C)nc34)[nH]n2)on1] | ¹H NMR (400 MHz, DMSO-d₆) 12.81 (s, 1H), 10.80 (s, 1H), 9.03 (s, 1H), 8.52 (s, 1H), 7.33 (s, 1H), 7.18 (d, J = 7.6 Hz, 1H), 6.27 (s, 1H), 4.14 (dq, J = 13.2, 6.6 Hz, 1H), 3.90 (s, 2H), 2.22 (s, 3H), 1.23 (d, J = 6.5 Hz, 6H). | 398 |
| N-(5-(2-(iso- propylamino) thieno[3,2- d]pyrimidin-7- yl)-1H-pyrazol- 3-yl)-3- (methoxy- methyl)- 1-methyl-1H- pyrazole-5- carboxamide | 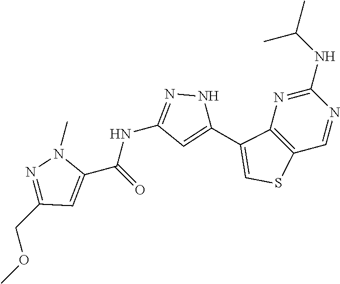 [SMILES: COCc1cc(C(=O)Nc2cc(-c3csc4cnc(NC(C)C)nc34)[nH]n2)n(C)n1] | ¹H NMR (400 MHz, DMSO-d₆) 12.90 (s, 1H), 10.88 (s, 1H), 9.04 (s, 1H), 8.56 (s, 1H), 7.48 (s, 1H), 7.18 (s, 2H), 4.37 (s, 2H), 4.17 (dt, J = 13.4, 6.8 Hz, 1H), 4.10 (s, 3H), 3.29 (s, 3H), 1.26 (d, J = 6.5 Hz, 6H). | 427 |
| 3- (methoxy- methyl)- 1-methyl-N-(5- (thieno[3,2-d] pyrimidin-7- yl)-1H-pyrazol- 3-yl)-1H- pyrazole-5- carboxamide | 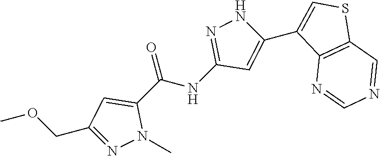 [SMILES: COCc1cc(C(=O)Nc2cc(-c3csc4cncnc34)[nH]n2)n(C)n1] | ¹H NMR (400 MHz, DMSO-d₆) 13.14 (s, 1H), 10.99 (s, 1H), 9.65 (s, 1H), 9.36 (s, 1H), 8.81 (s, 1H), 7.64 (s, 1H), 7.20 (s, 1H), 4.37 (s, 2H), 4.11 (s, 3H), 3.29 (s, 3H). | 370 |
| N-(5-(bicyclo [2.2.1]heptan-1- yl)-1H-pyrazol- 3-yl)-2-(3- methylisoxazol- 5-yl)acetamide | 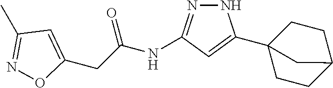 [SMILES: Cc1cc(CC(=O)Nc2cc(C34CCC(CC3)C4)[nH]n2)on1] | ¹H NMR (400 MHz, DMSO-d₆) 12.13 (s, 1H), 10.63 (s, 1H), 6.29 (d, J = 2.1 Hz, 1H), 6.22 (s, 1H), 3.83 (s, 2H), 2.30 (s, 1H), 2.20 (s, 3H), 1.77 ‥C 1.63 (m, 4H), 1.61 ‥C 1.47 (m, 4H), 1.40 ‥C 1.28 (m, 2H). | 301 |
| 3- (methoxy- methyl)- 1-methyl-N- (5-((1S,3R)-3- phenoxy- cyclopentyl)- 1H-pyrazol- 3-yl)- 1H-pyrazole-5- carboxamide | 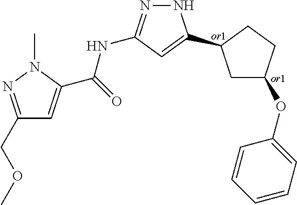 [SMILES: COCc1cc(C(=O)Nc2cc([C@H]3CC[C@@H](Oc4ccccc4)C3)[nH]n2)n(C)n1] | ¹H NMR (400 MHz, DMSO-d₆) 12.26 (d, J = 2.1 Hz, 1H), 10.72 (s, 1H), 7.34- 7.20 (m, 2H), 7.12 (s, 1H), 6.91 (dd, J = 8.4, 7.3 Hz, 3H), 6.45 (d, J = 2.2 Hz, 1H), 5.00-4.75 (m, 1H), 4.34 (s, 2H), 4.05 (s, 3H), 3.27 (s, 3H), 3.24-3.02 (m, 1H), 2.73-2.53 (m, 1H), 2.13-1.97 (m, 2H), 1.96-1.69 (m, 3H). | 396 |
| 3- (methoxy- methyl)- 1-methyl- N-(5-((1R,3S)- 3-phenoxy- cyclopentyl)- 1H-pyrazol-3- yl)-1H- pyrazole-5- carboxamide | 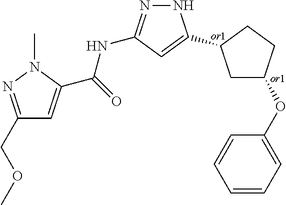 [SMILES: COCc1cc(C(=O)Nc2cc([C@@H]3CC[C@H](Oc4ccccc4)C3)[nH]n2)n(C)n1] | ¹H NMR (400 MHz, DMSO-d₆) 12.26 (s, 1H), 10.73 (s, 1H), 7.37-7.21 (m, 2H), 7.12 (s, 1H), 6.91 (dd, J = 8.4, 7.3 Hz, 3H), 6.44 (s, 1H), 4.99-4.80 (m, 1H), 4.34 (s, 2H), 4.05 (s, 3H), 3.27 (s, 3H), 3.14 (dq, J = 16.2, 8.3, 7.5 Hz, 1H), 2.71- 2.56 (m, 1H), 2.17-1.96 (m, 2H), 1.94- 1.64 (m, 3H). | 396 |
| N-(5-((1S,3R)- 3-((4- cyclopropyl- pyridin-3-yl) oxy) cyclopentyl)- 1H-pyrazol- 3-yl)-3- (methoxy- methyl)- | 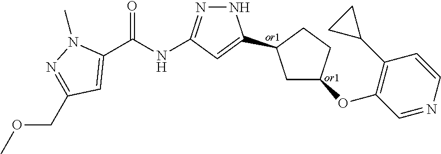 [SMILES: COCc1cc(C(=O)Nc2cc([C@H]3CC[C@@H](Oc4cnccc4C4CC4)C3)[nH]n2)n(C)n1] | ¹H NMR (400 MHz, DMSO-d₆) 12.26 (s, 1H), 10.72 (s, 1H), 8.21 (s, 1H), 8.03 (d, J = 4.9 Hz, 1H), 7.11 (s, 1H), 6.78 (d, J = 4.9 Hz, 1H), 6.47 (s, 1H), 5.09-5.02 (m, 1H), 4.33 (s, 2H), 4.04 (s, 3H), 3.27 (s, 3H), 3.19 (q, J = 8.2 Hz, 1H), 2.68- 2.58 (m, 1H), 2.13 (ddq, J = 24.3, 12.3, 5.2 Hz, 3H), 2.05-1.82 (m, 3H), 1.05- 0.96 (m, 2H), 0.76-0.68 (m, 2H). | 437 |
| 1-methyl- | | | |
| 1H-pyrazole-5- | | | |
| carboxamide | | | |
| N-(5-((1R,3S)- 3-((4- cyclopropyl- pyridin-3-yl) oxy) cyclopentyl)- 1H-pyrazol- 3-yl)-3- (methoxy- methyl)- 1-methyl- | 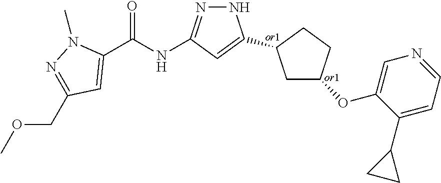 [SMILES: COCc1cc(C(=O)Nc2cc([C@@H]3CC[C@H](Oc4cnccc4C4CC4)C3)[nH]n2)n(C)n1] | ¹H NMR (400 MHz, DMSO-d₆) 12.26 (s, 1H), 10.72 (s, 1H), 8.21 (s, 1H), 8.03 (d, J = 4.9 Hz, 1H), 7.12 (s, 1H), 6.77 (d, J = 4.9 Hz, 1H), 6.47 (s, 1H), 5.14-4.99 (m, 1H), 4.33 (s, 2H), 4.04 (s, 3H), 3.27 (s, 3H), 3.19 (q, J = 8.2 Hz, 1H), 2.63 (dd, J = 14.5, 7.4 Hz, 1H), 2.25-2.01 (m, 3H), 2.01-1.79 (m, 3H), 1.10-0.94 (m, 2H), 0.83-0.67 (m, 2H). | 437 |
| 1H-pyrazole-5- | | | |
| carboxamide | | | |
| 3-(methoxy- methyl)- 1-methyl-N- (5-((1S,3R)- 3-((6- methylpyridin- 3-yl)oxy) cyclopentyl)- | 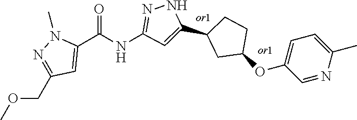 | ¹H NMR (400 MHz, Chloroform-d) 9.54 (s, 1H), 8.19 (d, J = 2.9 Hz, 1H), 7.21 (dd, J = 8.5, 2.8 Hz, 1H), 7.14 (d, J = 8.5 Hz, 1H), 6.82 (s, 1H), 6.71 (s, 1H), 4.52 (s, 2H), 4.19 (s, 3H), 3.48 (s, 3H), 3.27 (p, J = 7.6 Hz, 1H), 2.66-2.56 (m, 1H), 2.56 (s, 3H), 2.13 (s, 1H), 2.11-1.94 (m, 1H), 1.28 (s, 1H), 0.88 (s, 1H). | 411 |
| 1H-pyrazol- | | | |
| 3-yl)- | | | |
| 1H-pyrazole-5- | | | |
| carboxamide | | | |
| 3-(methoxy- methyl)- 1-methyl-N- (5-((1R,3S)- 3-((6- methylpyridin- 3-yl)oxy) cyclopentyl)- | 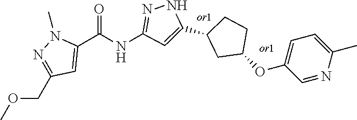 | ¹H NMR (400 MHz, CDCl₃) 1.95-2.14 (m, 4H), 2.19 (tt, J = 9.5, 4.5 Hz, 2H), 2.56 (s, 3H), 2.56-2.66 (m, 1H), 3.27 (p, J = 7.8 Hz, 1H), 3.48 (s, 3H), 4.19 (s, 3H), 4.52 (s, 2H), 4.88-4.94 (m, 1H), 6.71 (s, 1H), 6.83 (s, 1H), 7.14 (d, J = 8.5 Hz, 1H), 7.22 (dd, J = 8.5, 2.9 Hz, 1H), 8.20 (d, J = 2.8 Hz, 1H), 9.57 (s, 1H). | 411 |
| 1H-pyrazol- | | | |
| 3-yl)- | | | |
| 1H-pyrazole-5- | | | |
| carboxamide | | | |
| 2-(3- methylisoxazol- 5-yl)-N- (5-((1S,3R)-3- ((6-methyl- pyridazin-3- yl)oxy) cyclopentyl)- 1H-pyrazol-3- yl)acetamide | 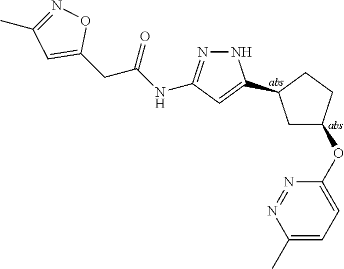 [SMILES: Cc1ccc(O[C@@H]2CC[C@H](c3cc(NC(=O)Cc4cc(C)no4)n[nH]3)C2)nn1] | ¹H NMR (400 MHz, Chloroform-d) 8.86 (s, 1H), 7.25 (d, J = 9.0 Hz, 1H), 6.90 (d, J = 9.0 Hz, 1H), 6.57 (s, 1H), 6.17 (s, 1H), 5.69 (s, 1H), 3.88 (s, 2H), 3.29 (p, J = 8.3 Hz, 1H), 2.73 (ddd, J = 14.9, 8.9, 6.4 Hz, 1H), 2.62 (s, 3H), 2.31 (s, 3H), 2.17 (ddt, J = 31.3, 9.9, 5.2 Hz, 3H), 2.06 ‥C 1.92 (m, 2H). | 383 |
| (1R,3S)-3- (3-((2-(methoxy- methyl) thiazolo[5,4- c]pyridin-4- yl)amino)-1H- pyrazol-5-yl) cyclopentyl isopropyl- carbamate | 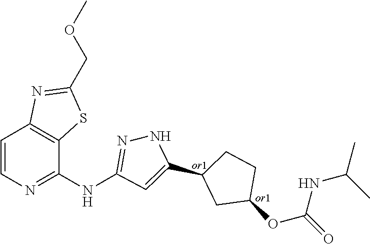 [SMILES: COCc1nc2ccnc(Nc3cc([C@H]4CC[C@@H](OC(=O)NC(C)C)C4)[nH]n3)c2s1] | ¹H NMR (400 MHz, DMSO-d₆) 12.28 (s, 1H), 9.78 (s, 1H), 8.15 (d, J = 5.8 Hz, 1H), 7.36 (d, J = 5.8 Hz, 1H), 6.95 (d, J = 7.8 Hz, 1H), 6.26 (s, 1H), 5.02 (s, 1H), 4.88 (s, 2H), 3.58 (q, J = 6.8 Hz, 1H), 3.48 (s, 3H), 3.15-3.06 (m, 1H), 2.92 (dt, J = 13.9, 7.2 Hz, 1H), 2.05 (q, J = 9.3 Hz, 1H), 1.99-1.86 (m, 1H), 1.75 (d, J = 8.7 Hz, 2H), 1.65 (s, 1H), 1.03 (dd, J = 6.6, 4.4 Hz, 6H). | 431 |
| (1S,3R)- 3-(3-((2- (methoxy- methyl) thiazolo[5,4- c]pyridin-4- yl)amino)-1H- pyrazol-5-yl) cyclopentyl isopropyl- carbamate | 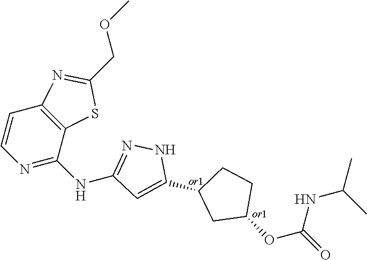 [SMILES: COCc1nc2ccnc(Nc3cc([C@@H]4CC[C@H](OC(=O)NC(C)C)C4)[nH]n3)c2s1] | ¹H NMR (400 MHz, DMSO-d₆) 12.28 (s, 1H), 9.92 (s, 1H), 8.16 (d, J = 5.9 Hz, 1H), 7.37 (d, J = 5.9 Hz, 1H), 6.95 (d, J = 7.9 Hz, 1H), 6.26 (s, 1H), 5.02 (s, 1H), 4.88 (s, 2H), 3.57 (dt, J = 13.6, 6.7 Hz, 1H), 3.48 (s, 3H), 3.16-3.07 (m, 1H), 2.92 (dq, J = 13.7, 7.2 Hz, 1H), 2.06 (q, J = 9.0 Hz, 1H), 1.93 (ddd, J = 13.7, 10.5, 7.5 Hz, 1H), 1.76 (s, 2H), 1.66 (s, 1H), 1.03 (dd, J = 6.6, 4.4 Hz, 6H). | 431 |
| (1R,3S)-3-(3- (pyrido[2,3- d]pyrimidin-4- ylamino)-1H- pyrazol-5-yl) cyclopentyl isopropyl- carbamate | 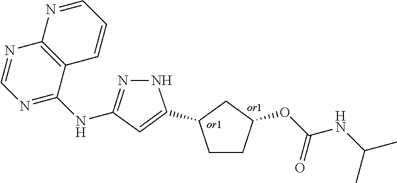 [SMILES: CC(C)NC(=O)O[C@@H]1CC[C@H](c2cc(Nc3ncnc4ncccc34)n[nH]2)C1] | ¹H NMR (400 MHz, DMSO-d₆) 12.33 (s, 1H), 10.68 (s, 1H), 9.11-9.02 (m, 2H), 8.74 (s, 1H), 7.60 (d, J = 7.6 Hz, 1H), 6.99 (d, J = 7.8 Hz, 1H), 6.69 (s, 1H), 5.02 (s, 1H), 3.63-3.53 (m, 1H), 3.12 (s, 1H), 2.51 (s, 1H), 2.10-2.02 (m, 1H), 1.93 (s, 1H), 1.76 (d, J = 8.5 Hz, 3H), 1.03 (dd, J = 6.6, 3.6 Hz, 6H). | 382 |
| (1S,3R)-3- (3-(pyrido[2,3- d]pyrimidin-4- ylamino)-1H- pyrazol-5-yl) cyclopentyl isopropyl- carbamate | 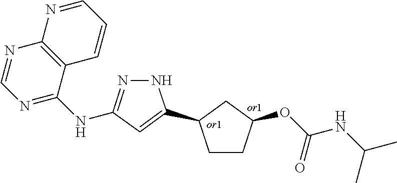 [SMILES: CC(C)NC(=O)O[C@H]1CC[C@@H](c2cc(Nc3ncnc4ncccc34)n[nH]2)C1] | ¹H NMR (400 MHz, DMSO-d₆) 12.33 (s, 1H), 10.67 (s, 1H), 9.06 (dd, J = 14.8, 6.0 Hz, 2H), 8.75 (s, 1H), 7.59 (dd, J = 8.2, 4.3 Hz, 1H), 6.99 (d, J = 7.9 Hz, 1H), 6.69 (s, 1H), 5.02 (s, 1H), 3.59 (dt, J = 13.8, 6.9 Hz, 1H), 3.12 (s, 1H), 2.51 (s, 1H), 2.07 (s, 1H), 1.91 (dd, J = 13.3, 7.0 Hz, 1H), 1.81-1.62 (m, 3H), 1.03 (dd, J = 6.6, 3.6 Hz, 6H) | 382 |
| (1R,3S)-3-(3- ((4-(4-methyl- 1H-pyrazol-1- yl)pyridin-2- yl)amino)-1H- pyrazol-5- yl)cyclopentyl isopropyl- carbamate | 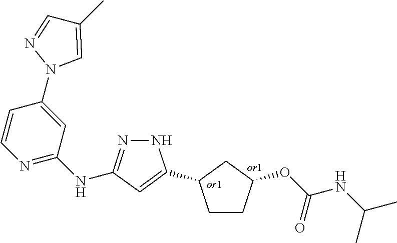 [SMILES: Cc1cnn(-c2ccnc(Nc3cc([C@H]4CC[C@@H](OC(=O)NC(C)C)C4)[nH]n3)c2)c1] | ¹H NMR (400 MHz, DMSO-d₆) 11.97 (s, 2H), 10.61 (s, 1H), 6.24 (d, J = 10.4 Hz, 2H), 3.83 (s, 2H), 3.14 (p, J = 7.9 Hz, 1H), 2.35 (dt, J = 15.0, 7.4Hz, 1H), 2.26 (d, J = 7.3 Hz, 2H), 2.20 (s, 3H), 2.04 (ddd, J = 15.4, 10.8, 6.8 Hz, 1H), 1.98- 1.87 (m, 1H), 1.87-1.76 (m, 1H), 1.61 (ddt, J = 29.4, 12.5, 8.1 Hz, 2H), 1.26 (dt, J = 12.2, 8.4 Hz, 1H). | 410 |
| (1S,3R)-3-(3- ((4-(4-methyl- 1H-pyrazol-1- yl)pyridin-2- yl)amino)-1H- pyrazol-5- yl)cyclopentyl isopropyl- carbamate | 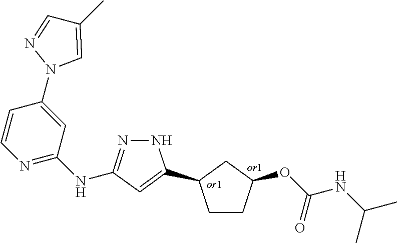 [SMILES: Cc1cnn(-c2ccnc(Nc3cc([C@@H]4CC[C@H](OC(=O)NC(C)C)C4)[nH]n3)c2)c1] | ¹H NMR (400 MHz, DMSO-d₆) 11.88 (s, 1H), 9.27 (s, 1H), 8.25 (s, 1H), 8.13 (d, J = 5.7 Hz, 1H), 7.86-7.81 (m, 1H), 7.63 (s, 1H), 7.08 (dd, J = 5.7, 2.0 Hz, 1H), 6.95 (d, J = 7.8 Hz, 1H), 6.04 (s, 1H), 5.01 (s, 1H), 3.63-3.54 (m, 1H), 3.10-3.01 (m, 1H), 2.49-2.41 (m, 1H), 2.11 (s, 3H), 2.02 (q, J = 8.2 Hz, 1H), 1.89 (ddt, J = 16.0, 11.1, 4.9 Hz, 1H), 1.73 (d, J = 9.2 Hz, 2H), 1.62 (s, 1H), 1.04 (d, J = 6.8 Hz, 6H). | 410 |
| N-(5-((1S,3R)- 3-((4- cyclopropyl- isothiazol- 3-yl)oxy) cyclopentyl)-1H- pyrazol-3-yl) thiazolo[5,4- c]pyridin- 4-amine | 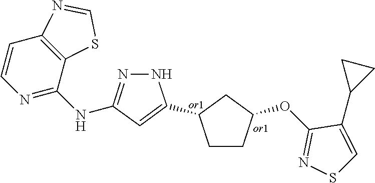 [SMILES: c1cc2ncsc2c(Nc2cc([C@H]3CC[C@@H](Oc4nscc4C4CC4)C3)[nH]n2)n1] | ¹H NMR (400 MHz, DMSO-d₆) 0.58- 0.67 (2H, m), 0.72-0.85 (2H, m), 1.73 (1H, tt), 1.81-2.19 (5H, m), 2.64 (1H, dt), 3.16-3.25 (1H, m), 5.35 (1H, s), 6.40 (1H, s), 7.40 (1H, d), 8.12 (1H, d), 8.26 (1H, s), 9.46 (2H, s), 12.14 (1H, s). | 425 |
| N-(5-((1R,3S)- 3-((4- cyclopropyl- isothiazol- 3-yl)oxy) cyclopentyl)-1H- pyrazol-3-yl) thiazolo[5,4- c]pyridin- 4-amine | 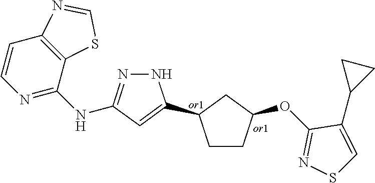 [SMILES: c1cc2ncsc2c(Nc2cc([C@@H]3CC[C@H](Oc4nscc4C4CC4)C3)[nH]n2)n1] | ¹H NMR (400 MHz, DMSO-d₆) 0.63 (2H, dd), 0.72-0.85 (2H, m), 1.72 (1H, td), 1.81-1.99 (3H, m), 1.99-2.17 (2H, m), 2.63 (1H, dd), 3.16-3.24 (1H, m), 5.35 (1H, s), 6.39 (1H, s), 7.40 (1H, d), 8.12 (1H, d), 8.27 (1H, s), 9.46 (2H, s), 12.12 (1H, s). | 425 |
| (1R,3S)-3- (3-(2-(3- methyl- isoxazol-5- yl)acetamido)- 1H-pyrazol-5- yl)cyclopentyl | 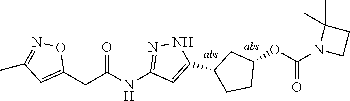 [SMILES: Cc1cc(CC(=O)Nc2cc([C@H]3CC[C@@H](OC(=O)N4CCC4(C)C)C3)[nH]n2)on1] | ¹H NMR (400 MHz, DMSO-d₆) 8.26- 7.90 (m, 1H), 6.74 (s, 1H), 6.26 (s, 1H), 3.94 (s, 2H), 3.14-3.02 (m, 1H), 2.20 (s, 3H), 1.97-1.88 (m, 2H), 1.67-1.55 (m, 6H). | 276.1 |
| 2,2- | | | |
| dimethyl- | | | |
| azetidine- | | | |
| 1-carboxylate | | | |
| N-(5-cyclo- pentyloxazol- 2-yl)-2-(3- methylisoxazol- 5-yl)acetamide | 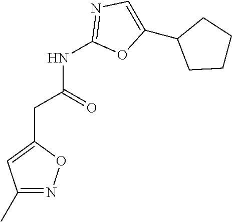 [SMILES: Cc1cc(CC(=O)Nc2ncc(C3CCCC3)o2)on1] | ¹H NMR (400 MHz, DMSO-d₆): 7.57 (s, 1H), 6.27 (s, 1H), 3.93 (s, 2H), 2.21 (s, 3H), 1.73-1.82 (m, 8H), 1.36 (s, 3H). | 306.1 |
| N-(5-(1- methyl- cyclopentyl) isothiazol- 3-yl)-2-(3- methyl- isoxazol-5- yl)acetamide | 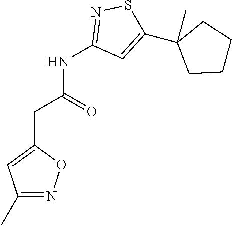 [SMILES: Cc1cc(CC(=O)Nc2cc(C3(C)CCCC3)sn2)on1] | ¹H NMR (400 MHz, DMSO-d₆) 12.07 (s, 1H), 10.61 (s, 1H), 7.99 (s, 1H), 6.24 (d, J = 14.6 Hz, 2H), 3.83 (s, 2H), 3.02 (dq, J = 10.7, 8.4 Hz, 1H), 2.20 (s, 4H), 2.10 (dt, J = 13.5, 7.0 Hz, 1H), 2.06- 1.92 (m, 3H), 1.77 (dq, J = 14.8, 7.7 Hz, 1H), 1.59 (dtd, J = 12.5, 9.3, 6.5 Hz, 1H), 1.31 (dtd, J = 12.9, 9.1, 5.9 Hz, 1H), 1.25 (s, 3H), 1.18 (dt, J = 12.3, 10.5 Hz, 1H), 0.59-0.52 (m, 2H), 0.52-0.44 (m, 2H). | 386 |
| N-(5- cyclopropyl- 1H-pyrazol- 3-yl)-2-(3- methylisoxazol- | 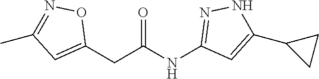 [SMILES: Cc1cc(CC(=O)Nc2cc(C3CC3)[nH]n2)on1] | ¹H NMR (400 MHz, CD₃OD): 6.26-6.28 (m, 2H), 2.32-3.34 (m, 2H), 2.29 (s, 3H), 2.03-2.07 (m, 2H), 1.69-1.80 (m, 6H), 1.34 (s, 3H). | 290.1 |
| 5-yl)acetamide | | | |
| N-(3-(1- methyl- cyclopentyl) isoxazol-5- yl)-2-(3- methylisoxazol- 5-yl)acetamide | 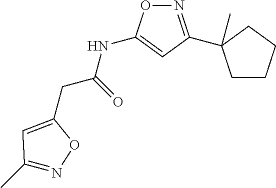 [SMILES: Cc1cc(CC(=O)Nc2cc(C3(C)CCCC3)no2)on1] | ¹H NMR (400 MHz, DMSO-d₆) 11.29 (br s, 1H), 10.45 (br s, 1H), 6.22 (s, 1H), 5.57 (br s, 1H), 3.82 (s, 2H), 3.13 (br s, 4H), 2.21 (s, 3H), 1.89 (br s, 4H) | 276.1 |
| 2-(3- methylisoxazol- 5-yl)-N- (5-(pyrrolidin- 1-yl)-1H- pyrazol-3-yl) acetamide | 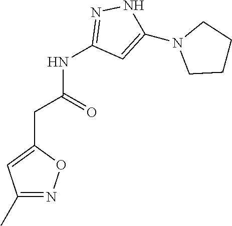 [SMILES: Cc1cc(CC(=O)Nc2cc(N3CCCC3)[nH]n2)on1] | ¹H NMR (400 MHz, DMSO-d₆) 12.06 (s, 1H), 10.62 (s, 1H), 7.32 (s, 1H), 6.27 (s, 1H), 6.22 (s, 1H), 4.74 (s, 1H), 3.83 (s, 2H), 2.97-2.66 (m, 1H), 2.21 (s, 3H), 1.94 (d, J = 7.4 Hz, 2H), 1.77 (s, 5H), 1.58 (d, J = 11.2 Hz, 2H), 1.39 (s, 1H), 1.24 (s, 3H), 0.59 (s, 2H), 0.47 (s, 2H). | 416 |
| (1R,3S)-3- (3-(2-(4- oxobenzo[d] [1,2,3]triazin- 3(4H)- yl)acetamido)- 1H-pyrazol-5-yl) cyclopentyl isopropyl- carbamate | 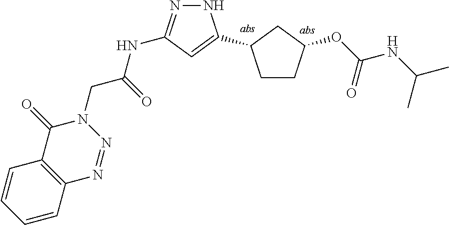 [SMILES: CC(C)NC(=O)O[C@@H]1CC[C@H](c2cc(NC(=O)Cn3nnc4ccccc4c3=O)n[nH]2)C1] | ¹H NMR (400 MHz, DMSO-d₆) 10.69 (s, 1H), 8.41 (s, 1H), 7.41 (s, 1H), 6.24 (s, 1H), 4.99 (s, 1H), 4.71 (q, J = 7.6 Hz, 1H), 3.87 (s, 2H), 2.60-2.52 (m, 1H), 2.21 (s, 3H), 2.11 (s, 1H), 1.95 (s, 2H), 1.80 (s, 1H), 1.23 (s, 3H), 0.59 (s, 2H), 0.47 (s, 2H). | 389 |
| N-(4- cyclopentyl-1H- imidazol-2- yl)-2-(3- methylisoxazol- | 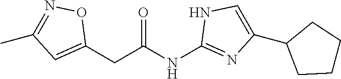 [SMILES: Cc1cc(CC(=O)Nc2nc(C3CCCC3)c[nH]2)on1] | ¹H NMR (400 MHz, DMSO-d₆) δ 12.05 (s, 1H), 10.63 (s, 1H), 7.48 (d, J = 7.8 Hz, 1H), 6.30 (s, 1H), 6.23 (s, 1H), 3.91- 3.72 (m, 3H), 2.82 (s, 1H), 2.20 (s, 4H), 1.93 (d, J = 15.1 Hz, 2H), 1.65 (t, J = 9.0 | 374 |
| 5-yl)acetamide | | Hz, 4H), 1.49 (q, J = 6.4, 4.2 Hz, 2H), | |
| | | 1.01 (d, J = 6.6 Hz, 6H). | |
| (S)-(1-(3-(2-(3- methylisoxazol- 5-yl)acetamido)- 1H-pyrazol-5- yl)pyrrolidin-3- yl)methyl (1-methyl- cyclopropyl) | 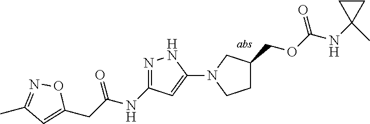 | ¹H NMR (400 MHz, METHANOL-d₄) δ 6.26 (s, 1H), 4.60 (s, 1H), 4.09-4.30 (m, 1H), 3.89 (s, 2H), 3.56 (br dd, J = 10.5, 4.8 Hz, 1H), 3.51-3.78 (m, 1H), 3.36 (br s, 2H), 3.24-3.31 (m, 1H), 2.29 (s, 2H), 2.24-2.31 (m, 1H), 2.02- 2.16 (m, 1H), 2.01-2.17 (m, 1H), 2.01- 2.19 (m, 1H), 1.33 (br s, 3H), 0.71 (br | 389.2 |
| carbamate | | s, 2H), 0.57 (br s, 2H). | |
| (S)-(1-(3-(2-(3- methylisoxazol- 5-yl)acetamido)- 1H-pyrazol-5- yl)azetidin-2- yl)methyl (1- methyl- cyclopropyl) carbamate | 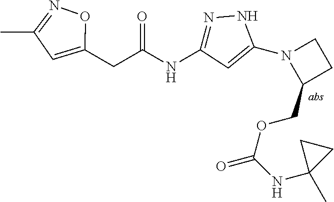 [SMILES: Cc1cc(CC(=O)Nc2cc(N3CC[C@H]3COC(=O)NC3(C)CC3)[nH]n2)on1] | ¹H NMR (400 MHz, DMSO-d₆) δ 11.32 (s, 1H), 10.34-10.61 (m, 1H), 7.44 (s, 1H), 6.22 (s, 1H), 5.56 (s, 1H), 3.87-3.98 (m, 2H), 3.81 (s, 2H), 3.11-3.28 (m, 3H), 2.94 (d, J = 6.4 Hz, 1H), 2.20 (s, 3H), 1.94-2.07 (m, 1H), 1.62-1.76 (m, 1H), 1.25 (s, 4H), 0.61 (s, 2H), 0.49 (s, 2H). | 403.2 |
| 2-(3- methylisoxazol- 5-yl)-N-(5- ((1S,3R)-3-((4- methylpyridazin- 3-yl)oxy) cyclopentyl)-1H- pyrazol-3-yl) acetamide | 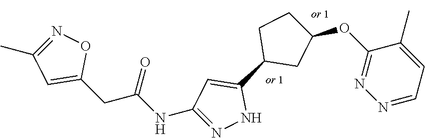 [SMILES: Cc1cc(CC(=O)Nc2cc([C@H]3CC[C@@H](Oc4nnccc4C)C3)[nH]n2)on1] | ¹H NMR (400 MHz, DMSO-d₆) 12.16 (s, 1H), 10.63 (s, 1H), 8.69 (d, J = 4.6 Hz, 1H), 7.44 (dd, J = 4.6, 1.1 Hz, 1H), 6.31 (s, 1H), 6.22 (s, 1H), 5.61 (q, J = 6.4, 5.2 Hz, 1H), 3.82 (s, 2H), 3.19 (p, J = 8.5 Hz, 1H), 2.75-2.60 (m, 1H), 2.20 (s, 3H), 2.13 (d, J = 1.0 Hz, 3H), 2.12- 2.03 (m, 2H), 1.94 (dd, J = 11.7, 8.7 Hz, 1H), 1.89-1.74 (m, 2H). | 383.15 |
| 2-(3- methylisoxazol- 5-yl)-N-(5- ((1R,3S)-3-((4- methylpyridazin- 3-yl)oxy) cyclopentyl)- 1H-pyrazol-3- yl)acetamide | 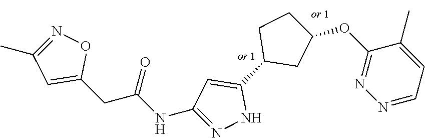 [SMILES: Cc1cc(CC(=O)Nc2cc([C@@H]3CC[C@H](Oc4nnccc4C)C3)[nH]n2)on1] | ¹H NMR (400 MHz, DMSO-d₆) 12.16 (s, 1H), 10.63 (s, 1H), 8.70 (d, J = 4.6 Hz, 1H), 7.44 (dd, J = 4.6, 1.1 Hz, 1H), 6.31 (s, 1H), 6.22 (s, 1H), 5.62 (dq, J = 6.4, 3.3, 2.7 Hz, 1H), 3.83 (s, 2H), 3.19 (p, J = 8.6 Hz, 1H), 2.64 (ddd, J = 14.6, 8.6, 6.6 Hz, 1H), 2.20 (s, 3H), 2.13 (d, J = 1.0 Hz, 3H), 2.12-2.03 (m, 2H), 1.94 (dd, J = 11.7, 8.9 Hz, 1H), 1.83 (tdd, J = | 383.10 |
| | | 9.1, 5.9, 2.6 Hz, 2H). | |
| (1R,3S)-3- (3-(2-(3- methylisoxazol- 5-yl)acetamido)- 1H-pyrazol-5- yl)cyclopentyl 3- methylbutanoate | 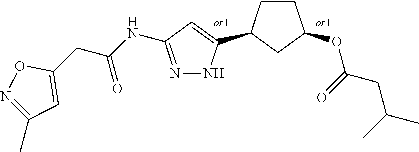 [SMILES: Cc1cc(CC(=O)Nc2cc([C@H]3CC[C@@H](OC(=O)CC(C)C)C3)[nH]n2)on1] | ¹H NMR (400 MHz, Chloroform-d) 8.90 (s, 1H), 6.52 (s, 1H), 6.19 (s, 1H), 5.31- 5.24 (m, 1H), 3.92 (s, 2H), 3.28 (p, J = 8.3 Hz, 1H), 2.58 (ddd, J = 15.0, 9.0, 6.4 Hz, 1H), 2.33 (s, 3H), 2.21 (t, J = 1.8 Hz, 1H), 2.21-2.08 (m, 2H), 2.11-1.95 (m, 1H), 1.97-1.86 (m, 2H), 1.89-1.78 (m, 1H), 0.97 (d, J = 6.6 Hz, 6H) | 375.15 |
| (1S,3R)-3- (3-(2-(3- methylisoxazol- 5-yl)acetamido)- 1H-pyrazol-5- yl)cyclopentyl 3- methylbutanoate | 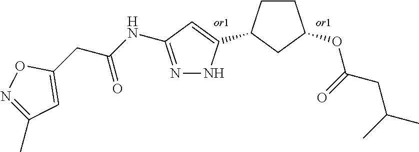 [SMILES: Cc1cc(CC(=O)Nc2cc([C@@H]3CC[C@H](OC(=O)CC(C)C)C3)[nH]n2)on1] | ¹H NMR (400 MHz, Chloroform-d) 8.65 (s, 1H), 6.52 (s, 1H), 6.18 (s, 1H), 5.28 (tt, J = 6.6, 3.5 Hz, 1H), 3.90 (s, 2H), 3.24 (p, J = 8.4 Hz, 1H), 2.56 (ddd, J = 14.9, 9.0, 6.5 Hz, 1H), 2.32 (s, 3H), 2.23- 2.13 (m, 3H), 2.17-2.00 (m, 1H), 2.04- 1.78 (m, 3H), 0.97 (d, J = 6.7 Hz, 6H) | 375.20 |
| N-(3-((1S,3R)- 3-((4- isopropyl-4H- 1,2,4-triazol-3- yl)methyl) cyclopentyl)-1H- pyrazol-5-yl)-2- (3- | 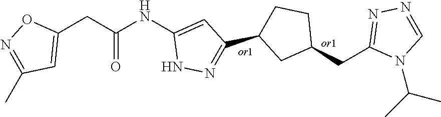 [SMILES: Cc1cc(CC(=O)Nc2cc([C@H]3CC[C@@H](Cc4nncn4C(C)C)C3)n[nH]2)on1] | ¹H NMR (400 MHz, DMSO-d₆) 12.10 (s, 1H), 10.63 (s, 1H), 8.56 (s, 1H), 6.25 (d, J = 20.0 Hz, 2H), 4.36 (hept, J = 6.6 Hz, 1H), 3.83 (s, 2H), 3.06 (p, J = 8.5 Hz, 1H), 2.77 (d, J = 7.3 Hz, 2H), 2.43 (dt, J = 17.4, 7.7 Hz, 1H), 2.20 (s, 4H), 1.94-2.07 (m, 1H), 1.79-1.93 (m, 1H), 1.67 (dtd, J = 12.3, 9.3, 6.6 Hz, 1H), | 398.10 |
| methylisoxazol- | | 1.41-1.52 (m, 1H), 1.27-1.4 (m, 7H). | |
| 5-yl)acetamide | | | |
| N-(3- ((1R,3S)-3-((4- isopropyl-4H- 1,2,4-triazol-3- yl)methyl) cyclopentyl)- 1H-pyrazol-5- yl)-2-(3- | 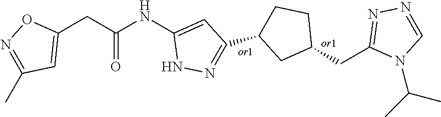 [SMILES: Cc1cc(CC(=O)Nc2cc([C@@H]3CC[C@H](Cc4nncn4C(C)C)C3)n[nH]2)on1] | ¹H NMR (400 MHz, DMSO-d₆) 12.09 (s, 1H), 10.62 (s, 1H), 8.56 (s, 1H), 6.24 (d, J = 13.9 Hz, 2H), 4.37 (hept, J = 6.7 Hz, 1H), 3.82 (s, 2H), 3.19 (p, J = 7.9 Hz, 1H), 2.75 (d, J = 7.5 Hz, 2H), 2.50 (s, 1H), 2.20 (s, 3H), 2.08 (dtd, J = 10.9, 7.4, 3.3 Hz, 1H), 1.93 (dtd, J = 11.1, 7.5, 3.4 Hz, 1H), 1.79 (ddt, J = 19.4, 8.5, 7.0 | 398.10 |
| methylisoxazol- | | Hz, 2H), 1.53-1.67 (m, 1H), 1.28-1.42 | |
| 5-yl)acetamide | | (m, 7H). | |
| N-(3- ((1R,3R)-3-((4- isopropyl-4H- 1,2,4-triazol-3- yl)methyl) cyclopentyl)- 1H-pyrazol-5- yl)-2-(3- | 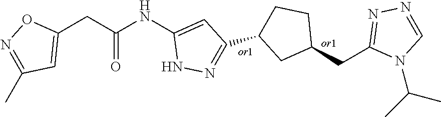 [SMILES: Cc1cc(CC(=O)Nc2cc([C@@H]3CC[C@@H](Cc4nncn4C(C)C)C3)n[nH]2)on1] | ¹H NMR (400 MHz, DMSO-d₆) 12.12 (s, 1H), 10.68 (s, 1H), 8.56 (s, 1H), 6.25 (d, J = 21.5 Hz, 2H), 4.37 (h, J = 6.7 Hz, 1H), 3.84 (s, 2H), 3.06 (dq, J = 10.6, 8.3 Hz, 1H), 2.77 (d, J = 7.3 Hz, 2H), 2.35- 2.49 (m, 1H), 2.20 (s, 4H), 2.00 (ddt, J = 13.3, 7.9, 4.1 Hz, 1H), 1.86 (dq, J = 15.1, 7.7 Hz, 1H), 1.67 (dtd, J = 12.3, 9.3, 6.6 | 398.10 |
| methylisoxazol- | | Hz, 1H), 1.41-1.52 (m, 1H), 1.27-1.41 | |
| 5-yl)acetamide | | (m, 7H). | |
| N-(3-((1S,3S)- 3-((4-isopropyl- 4H-1,2,4-triazol- 3-yl)methyl) cyclopentyl)- 1H-pyrazol- 5-yl)-2-(3- methylisoxazol- | 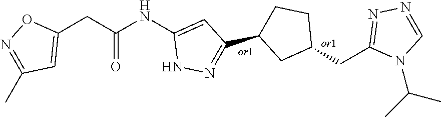 [SMILES: Cc1cc(CC(=O)Nc2cc([C@H]3CC[C@H](Cc4nncn4C(C)C)C3)n[nH]2)on1] | ¹H NMR (400 MHz, DMSO-d₆) 12.10 (s, 1H), 10.63 (s, 1H), 8.56 (s, 1H), 6.24 (d, J = 14.5 Hz, 2H), 4.37 (hept, J = 6.7 Hz, 1H), 3.83 (s, 2H), 3.20 (p, J = 7.9 Hz, 1H), 2.75 (d, J = 7.5 Hz, 2H), 2.50 (d, J = 1.8 Hz, 1H), 2.20 (s, 3H), 2.08 (dtd, J = 11.1, 7.5, 3.3Hz, 1H), 1.93 (dtd, J = 15.6, 7.5, 3.4 Hz, 1H), 1.69-1.87 (m, | 398.15 |
| 5-yl)acetamide | | 2H), 1.61 (tt, J = 12.3, 8.1 Hz, 1H), 1.28- | |
| | | 1.41 (m, 7H). | |

### Example 19

### rel-(3S,5R)-5-(3-(2-(3-methylisoxazol-5-yl) acetamido)-1H-pyrazol-5-yl) tetrahydrofuran-3-yl (1-methylcyclopropyl) carbamate

### rel-(3R,5R)-5-(3-(2-(3-methylisoxazol-5-yl) acetamido)-1H-pyrazol-5-yl)tetrahydrofuran-3-yl 2-(1-methylcyclopropyl)acetate

### (3S,5R)-5-(3-(2-(3-methylisoxazol-5-yl) acetamido)-1H-pyrazol-5-yl)tetrahydrofuran-3-yl (1-methylcyclopropyl)carbamate

### (3R,5S)-5-(3-(2-(3-methylisoxazol-5-yl) acetamido)-1H-pyrazol-5-yl) tetrahydrofuran-3-yl

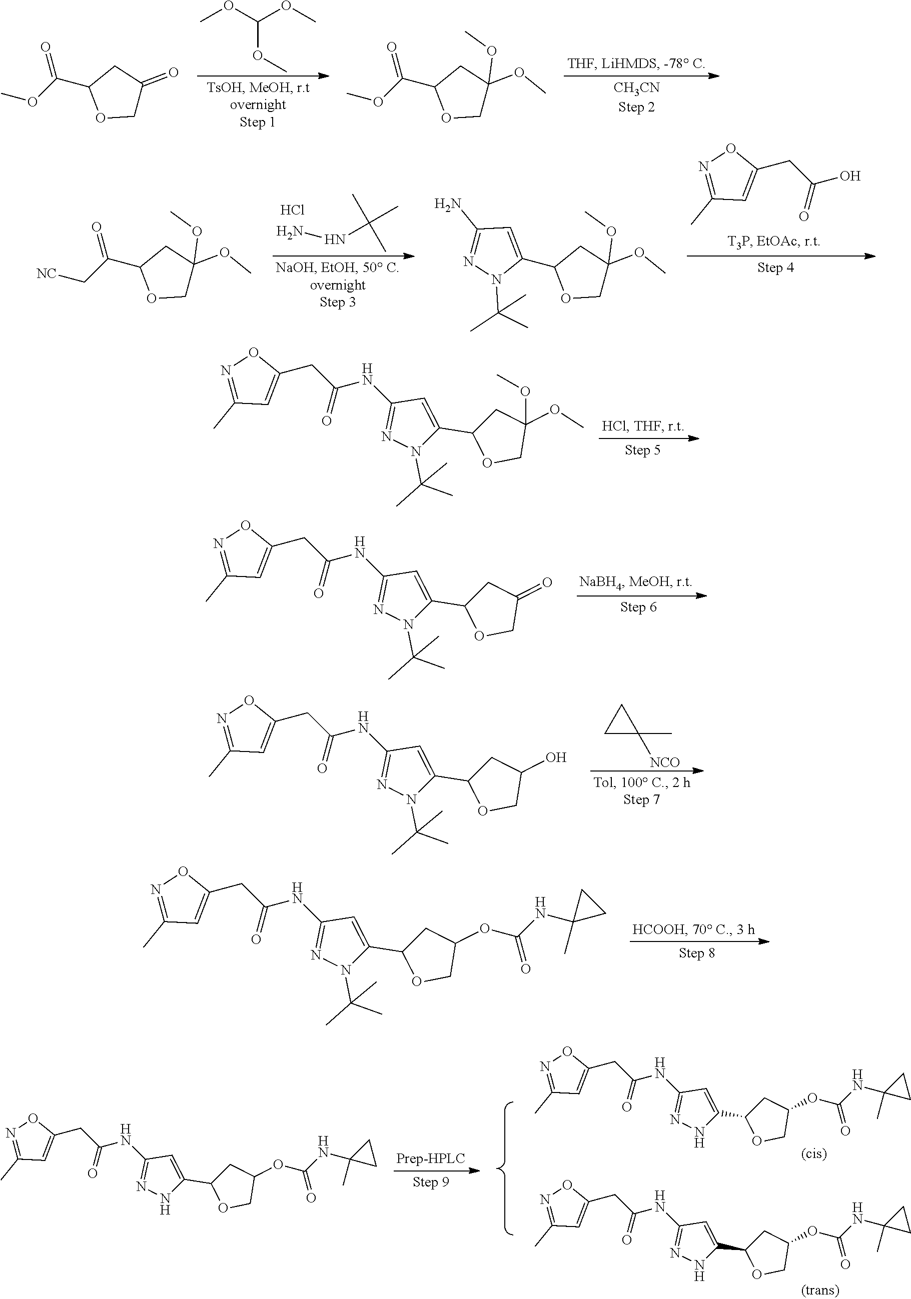

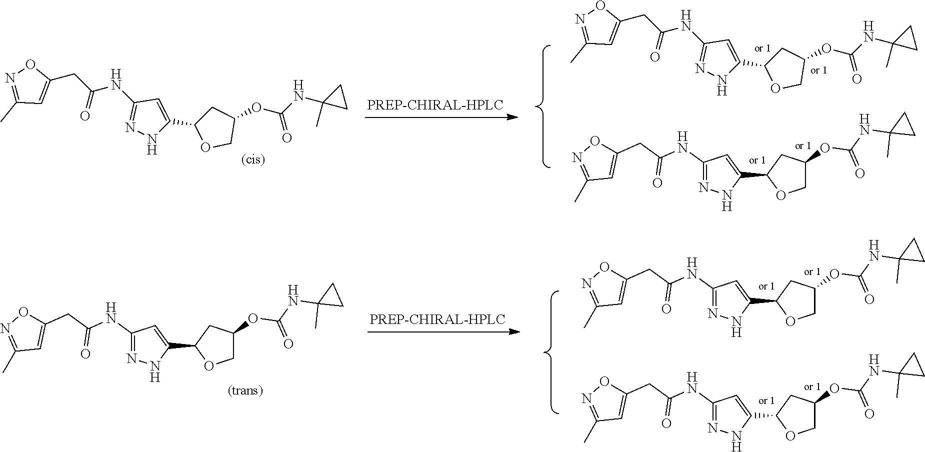 [SMILES: Cc1cc(CC(=O)Nc2cc([C@@H]3C[C@H](OC(=O)NC4(C)CC4)CO3)[nH]n2)on1 ; Cc1cc(CC(=O)Nc2cc([C@@H]3C[C@H](OC(=O)NC4(C)CC4)CO3)[nH]n2)on1 ; Cc1cc(CC(=O)Nc2cc([C@H]3C[C@@H](OC(=O)NC4(C)CC4)CO3)[nH]n2)on1 ; Cc1cc(CC(=O)Nc2cc([C@H]3C[C@@H](OC(=O)NC4(C)CC4)CO3)[nH]n2)on1 ; Cc1cc(CC(=O)Nc2cc([C@@H]3C[C@@H](OC(=O)NC4(C)CC4)CO3)[nH]n2)on1 ; Cc1cc(CC(=O)Nc2cc([C@H]3C[C@H](OC(=O)NC4(C)CC4)CO3)[nH]n2)on1]

### methyl 4,4-dimethoxytetrahydrofuran-2-carboxylate

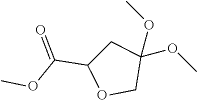

Step 1: A round bottomed flask was charged with methyl 4-oxotetrahydrofuran-2-carboxylate (2 g, 0.01 mol), trimethoxymethane (9 g, 0.08 mol), TsOH (0.05 g, 0.3 mmol), MeOH (20 mL) and a stirbar. The solution was stirred for 16 hours at 24° C. The product was detected by TLC. The mixture was quenched with saturated NaHCO₃(aq.), then concentrated under vacuum to remove most of the methanol. The reaction mixture was diluted with water (50 mL), and the aqueous phase was extracted with EA (50 mL) three times. The combined organic layers were washed with brine, dried over sodium sulfate, filtered, and concentrated in vacuo to give methyl 4,4-dimethoxytetrahydrofuran-2-carboxylate (1.5 g, 7.9 mmol, 60%) as a yellow oil.

m/z (ES⁺): no MS signal.

### 3-(4,4-dimethoxytetrahydrofuran-2-yl)-3-oxopropanenitrile

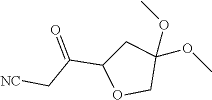

Step 2: To a mixture of methyl 4,4-dimethoxytetrahydrofuran-2-carboxylate (1.4 g, 7.4 mmol) and CH₃CN (0.91 g, 22 mmol) in THE (15 mL) was added lithium bis(trimethylsilyl)amide (15 mL, 1 M in THF, 15 mmol) dropwise at −78° C. under nitrogen atmosphere. The mixture was stirred for 1.5 hour at −78° C. The reaction mixture was detected by TLC. The reaction was quenched with NH₄Cl (sat. aq, 15 mL), and extracted with EA (3*20 mL). The combined organic layers were washed with brine (2*10 mL), dried over Na₂SO₄, filtered, and concentrated under vacuum. The residue was purified by reverse flash chromatography with the following conditions: column, C18 silica gel; mobile phase, MeCN in water, 0% to 100% gradient in 20 min; detector, UV 220 nm to give 3-(4,4-dimethoxytetrahydrofuran-2-yl)-3-oxopropanenitrile (970 mg, 4.87 mmol, 66.4%) as a yellow oil.

m/z (ES⁺) [M+H]⁺=no signal.

### 1-(tert-butyl)-5-(4,4-dimethoxytetrahydrofuran-2-yl)-1H-pyrazol-3-amine

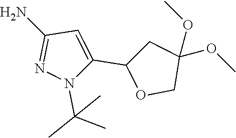 [SMILES: COC1(OC)COC(c2cc(N)nn2C(C)(C)C)C1]

Step 3: Sodium hydroxide (1.03 g, 25.7 mmol) was added in portions to a suspension of tert-butylhydrazine hydrochloride (3.23 g, 25.8 mmol) in EtOH (40 mL) at room-temperature, and stirred at room temperature for 1 hour. A solution of 3-(4,4-dimethoxytetrahydrofuran-2-yl)-3-oxopropanenitrile (4.25 g, 21.3 mmol) in ethanol was added at room temperature, then the mixture was heated to 50° C. internal and stirred for overnight. The mixture was filtered, and concentrated in vacuo. The residue was purified by reverse flash chromatography with the following conditions: column, C18 silica gel; mobile phase, MeCN in water, 30% to 60% gradient in 20 min; detector, UV 254 nm. Concentration in vacuo resulted in 1-(tert-butyl)-5-(4,4-dimethoxytetrahydrofuran-2-yl)-1H-pyrazol-3-amine (1.2 g, 4.5 mmol, 21%) as a yellow oil.

m/z (ES⁺) [M+H]⁺=270.20; HPLC tR=0.892 min.

### N-(1-(tert-butyl)-5-(4,4-dimethoxytetrahydrofuran-2-yl)-1H-pyrazol-3-yl)-2-(3-methylisoxazol-5-yl)acetamide

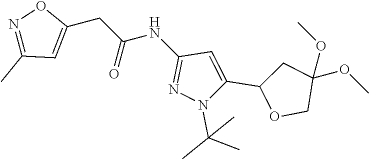

Step 4: To a mixture of 2-(3-methylisoxazol-5-yl) acetic acid (635 mg, 4.50 mmol), 1-(tert-butyl)-5-(4,4-dimethoxytetrahydrofuran-2-yl)-1H-pyrazol-3-amine (1.21 g, 4.50 mmol) and DIEA (1.74 g, 13.5 mmol) in EA (15 mL) was added 2,4,6-tripropyl-1,3,5,2,4,6-trioxatriphosphinane 2,4,6-trioxide (4.29 g, 50% Wt solution in ethyl acetate, 6.75 mmol) dropwise at 0° C. under nitrogen atmosphere. The mixture was stirred for 2 hours at 25° C. The reaction was quenched with sat. aq Na₂CO₃ (10 mL) and extracted with EA (2*10 mL). The organic layer was washed with more aq·Na₂CO₃ (2*10 mL) and brine (30 mL), and concentrated. The residue was purified by reverse flash chromatography with the following conditions: column, C18 silica gel; mobile phase, MeCN in water, 0% to 100% gradient in 15 min; detector, UV 220 nm to afford N-(1-(tert-butyl)-5-(4,4-dimethoxytetrahydrofuran-2-yl)-1H-pyrazol-3-yl)-2-(3-methylisoxazol-5-yl) acetamide (920 mg, 52%) as white solid.

m/z (ES⁺) [M+H]⁺=393.25; HPLC tR=0.945 min

### N-(1-(tert-butyl)-5-(4-oxotetrahydrofuran-2-yl)-1H-pyrazol-3-yl)-2-(3-methylisoxazol-5-yl)acetamide

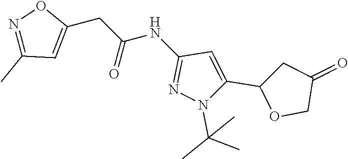 [SMILES: Cc1cc(CC(=O)Nc2cc(C3CC(=O)CO3)n(C(C)(C)C)n2)on1]

Step 5: A round bottomed flask was charged with N-(1-(tert-butyl)-5-(4,4-dimethoxytetrahydrofuran-2-yl)-1H-pyrazol-3-yl)-2-(3-methylisoxazol-5-yl) acetamide (910 mg, 2.32 mmol) and a stirbar. HCl (3 mol/L)/THF (10 mL) was added, and the solution was stirred for 2 hours at 24° C. The reaction mixture was diluted with water (20 mL), and the aqueous phase was extracted with DCM (20 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 10% to 50% gradient in 15 min; detector, UV 254 nm to afford N-(1-(tert-butyl)-5-(4-oxotetrahydrofuran-2-yl)-1H-pyrazol-3-yl)-2-(3-methylisoxazol-5-yl) acetamide (803 mg, 2.32 mmol, 100%) as a yellow oil.

m/z (ES⁺) [M+H]⁺=347.30; HPLC tR=0.813 min.

### N-(1-(tert-butyl)-5-(4-hydroxytetrahydrofuran-2-yl)-1H-pyrazol-3-yl)-2-(3-methylisoxazol-5-yl)acetamide

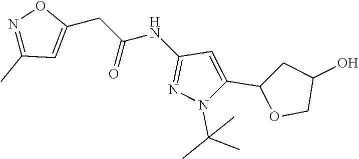 [SMILES: Cc1cc(CC(=O)Nc2cc(C3CC(O)CO3)n(C(C)(C)C)n2)on1]

Step 6: A round bottomed flask was charged with N-(1-(tert-butyl)-5-(4-oxotetrahydrofuran-2-yl)-1H-pyrazol-3-yl)-2-(3-methylisoxazol-5-yl) acetamide (270 mg, 779 μmol), MeOH (5 mL), and a stirbar. To the above mixture, NaBH₄ (88.5 mg, 2.34 mmol) was added. The resulting solution was stirred for 2 h at 24° C. The mixture was quenched with water and concentrated under vacuum to remove most of the MeOH. The reaction mixture was diluted with water (30 mL), and the aqueous phase was extracted with DCM (20 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 10% to 40% gradient in 15 min; detector, UV 254 nm to afford N-(1-(tert-butyl)-5-(4-hydroxytetrahydrofuran-2-yl)-1H-pyrazol-3-yl)-2-(3-methylisoxazol-5-yl) acetamide (180 mg, 517 μmol, 66.3%) as a yellow oil.

m/z (ES⁺) [M+H]⁺=349.30; HPLC tR=0.772 min.

### 5-(1-(tert-butyl)-3-(2-(3-methylisoxazol-5-yl)acetamido)-1H-pyrazol-5-yl)tetrahydrofuran-3-yl (1-methylcyclopropyl)carbamate

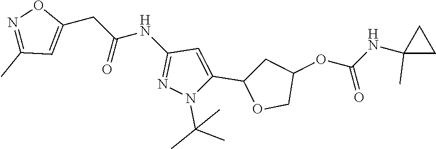 [SMILES: Cc1cc(CC(=O)Nc2cc(C3CC(OC(=O)NC4(C)CC4)CO3)n(C(C)(C)C)n2)on1]

Step 7: To a stirred mixture of N-(1-(tert-butyl)-5-(4-hydroxytetrahydrofuran-2-yl)-1H-pyrazol-3-yl)-2-(3-methylisoxazol-5-yl) acetamide (160 mg, 459 μmol) and 1-isocyanato-1-methylcyclopropane (0.6 M in toluene) (2.3 mL, 1.38 mmol) was added DIEA (178 mg, 240 μL, 1.38 mmol) dropwise at room temperature under nitrogen atmosphere. The resulting mixture was stirred for 2 hours at 100° C. under nitrogen atmosphere. Then the reaction mixture was allowed to cool down to room temperature and concentrated under reduced pressure. The residue was purified by reverse phase flash to afford 5-(1-(tert-butyl)-3-(2-(3-methylisoxazol-5-yl) acetamido)-1H-pyrazol-5-yl) tetrahydrofuran-3-yl (1-methylcyclopropyl) carbamate (145 mg, 325 μmol, 70.9%) as a light yellow oil.

m/z (ES⁺) [M+H]⁺=446.30; HPLC tR=0.952 min

### 5-(3-(2-(3-methylisoxazol-5-yl)acetamido)-1H-pyrazol-5-yl)tetrahydrofuran-3-yl (1-methylcyclopropyl)carbamate

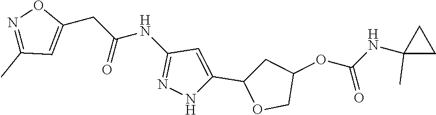 [SMILES: Cc1cc(CC(=O)Nc2cc(C3CC(OC(=O)NC4(C)CC4)CO3)[nH]n2)on1]

Step 8: A round bottomed flask was charged with 5-(1-(tert-butyl)-3-(2-(3-methylisoxazol-5-yl)acetamido)-1H-pyrazol-5-yl)tetrahydrofuran-3-yl (1-methylcyclopropyl)carbamate (170 mg, 382 μmol) and a stirbar. HCOOH (5 mL) was added, and the solution was stirred for 3 hours at 70° C. and concentrated under vacuum to remove HCOOH. The residue was purified by reverse flash chromatography with the following conditions: column, C18 silica gel; mobile phase, MeCN in water, 10% to 50% gradient in 10 min; detector, UV 254 nm to afford 5-(3-(2-(3-methylisoxazol-5-yl)acetamido)-1H-pyrazol-5-yl)tetrahydrofuran-3-yl (1-methylcyclopropyl)carbamate (100 mg, 257 μmol, 67.3%).

m/z (ES⁺) [M+H]⁺=390.15; HPLC tR=0.887, 0.961 min.

### (cis) (3S,5S)-5-(3-(2-(3-methylisoxazol-5-yl)acetamido)-1H-pyrazol-5-yl)tetrahydrofuran-3-yl (1-methylcyclopropyl)carbamate and trans (3S,5R)-5-(3-(2-(3-methylisoxazol-5-yl)acetamido)-1H-pyrazol-5-yl)tetrahydrofuran-3-yl (1-methylcyclopropyl)carbamate

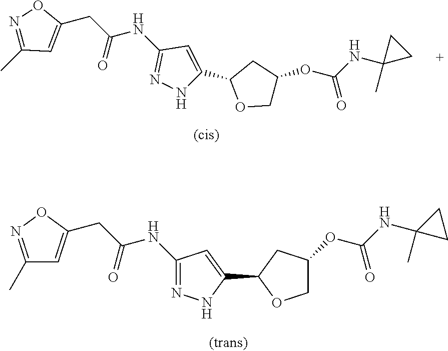 [SMILES: Cc1cc(CC(=O)Nc2cc([C@@H]3C[C@H](OC(=O)NC4(C)CC4)CO3)[nH]n2)on1 ; Cc1cc(CC(=O)Nc2cc([C@H]3C[C@H](OC(=O)NC4(C)CC4)CO3)[nH]n2)on1]

Step 9: The 5-(3-(2-(3-methylisoxazol-5-yl) acetamido)-1H-pyrazol-5-yl) tetrahydrofuran-3-yl (1-methylcyclopropyl) carbamate (100 mg, 57 μmol) was purified by Pre-HPLC (Column: XB ridge Prep OBD C18 Column, 30*150 mm, 5 m; Mobile Phase A: Water(10 mmol/L NH₄HCO₃), Mobile Phase B: ACN; Flow rate: 60 mL/min; Gradient: 10% B to 32% B in 8 min, 32% B; Wave Length: 220 nm; RT1(min): 7.55/8.27). Lyophilization yielded cis-5-(3-(2-(3-methylisoxazol-5-yl) acetamido)-1H-pyrazol-5-yl) tetrahydrofuran-3-yl (1-methylcyclopropyl) carbamate (50 mg, 0.13 mmol, 50%) as a white amorphous solid and trans-5-(3-(2-(3-methylisoxazol-5-yl) acetamido)-1H-pyrazol-5-yl) tetrahydrofuran-3-yl (1-methylcyclopropyl) carbamate (10 mg, 26 μmol, 10%) as a white amorphous solid.

(cis) m/z (ES⁺) [M+H]=390.30; HPLC tR=0.746 min.

(trans) m/z (ES⁺) [M+H]⁺=390.30; HPLC tR=0.780 min.

### rel-(3S,5R)-5-(3-(2-(3-methylisoxazol-5-yl) acetamido)-1H-pyrazol-5-yl) tetrahydrofuran-3-yl (1-methylcyclopropyl) carbamate

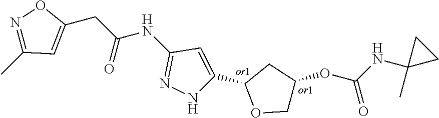 [SMILES: Cc1cc(CC(=O)Nc2cc([C@@H]3C[C@H](OC(=O)NC4(C)CC4)CO3)[nH]n2)on1]

Step 10: The compound cis-5-(3-(2-(3-methylisoxazol-5-yl) acetamido)-1H-pyrazol-5-yl) tetrahydrofuran-3-yl (1-methylcyclopropyl) carbamate (50 mg, 0.13 mmol) was purified by PREP-CHIRAL-HPLC (Column: CHIRALPAK IG, 2*25 cm, 5 m; Mobile Phase A: Hex(0.2% DEA)—HPLC, Mobile Phase B: EtOH: DCM=1:1—HPLC; Flow rate: 20 mL/min; Gradient: 80% B to 80% B in 11.5 min; Wave Length: 220/254 nm; RT1(min): 4.02; Sample Solvent: EtOH: DCM=1:1—HPLC; Injection Volume: 1.7 mL; Number Of Runs: 1). Lyophilization yielded rel-(3S,5R)-5-(3-(2-(3-methylisoxazol-5-yl) acetamido)-1H-pyrazol-5-yl) tetrahydrofuran-3-yl (1-methylcyclopropyl) carbamate (13 mg, 33 μmol, 52%) as a white amorphous solid.

m/z (ES⁺) [M+H]⁺=390.15; HPLC tR=0.954 min.

¹H NMR (400 MHz, Chloroform-d) 8.89 (s, 1H), 6.62 (s, 1H), 6.18 (s, 1H), 5.36 (s, 2H), 5.13 (s, 1H), 4.12 (d, J=10.7 Hz, 1H), 3.98 (s, 1H), 3.90 (s, 2H), 2.67 (s, 1H), 2.33 (s, 3H), 2.20 (d, J=14.1 Hz, 1H), 1.34 (s, 3H), 0.74 (s, 2H), 0.60 (s, 2H).

### rel-(3R,5R)-5-(3-(2-(3-methylisoxazol-5-yl) acetamido)-1H-pyrazol-5-yl)tetrahydrofuran-3-yl 2-(1-methylcyclopropyl)acetate

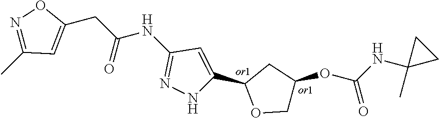 [SMILES: Cc1cc(CC(=O)Nc2cc([C@H]3C[C@@H](OC(=O)NC4(C)CC4)CO3)[nH]n2)on1]

The compound cis-5-(3-(2-(3-methylisoxazol-5-yl) acetamido)-1H-pyrazol-5-yl)tetrahydrofuran-3-yl (1-methylcyclopropyl)carbamate (50 mg, 0.13 mmol) was purified by PREP-CHIRAL-HPLC (Column: CHIRALPAK IG, 2*25 cm, 5 m; Mobile Phase A: Hex(0.2% DEA)—HPLC, Mobile Phase B: EtOH: DCM=1:1—HPLC; Flow rate: 20 mL/min; Gradient: 80% B to 80% B in 11.5 min; Wave Length: 220/254 nm; RT2(min): 8.23; Sample Solvent: EtOH: DCM=1:1—HPLC; Injection Volume: 1.7 mL; Number Of Runs: 1). Lyophilization yielded rel-(3R,5R)-5-(3-(2-(3-methylisoxazol-5-yl) acetamido)-1H-pyrazol-5-yl)tetrahydrofuran-3-yl 2-(1-methylcyclopropyl)acetate (12.3 mg, 31.7 μmol, 49%) as a white amorphous solid.

m/z (ES⁺) [M+H]⁺=390.20; HPLC tR=0.961 min.

¹H NMR (400 MHz, Chloroform-d) 9.01 (s, 1H), 6.62 (s, 1H), 6.18 (s, 1H), 5.40 (s, 1H), 5.35 (s, 1H), 5.13 (s, 1H), 4.12 (d, J=10.8 Hz, 1H), 3.97 (d, J=11.0 Hz, 1H), 3.90 (s, 2H), 2.68 (s, 1H), 2.33 (s, 3H), 2.20 (d, J=13.9 Hz, 1H), 1.34 (s, 3H), 0.73 (s, 2H), 0.59 (s, 2H).

### (3S,5R)-5-(3-(2-(3-methylisoxazol-5-yl) acetamido)-1H-pyrazol-5-yl)tetrahydrofuran-3-yl (1-methylcyclopropyl)carbamate

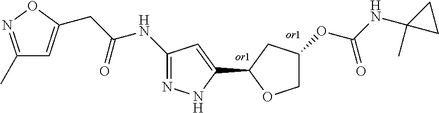 [SMILES: Cc1cc(CC(=O)Nc2cc([C@H]3C[C@H](OC(=O)NC4(C)CC4)CO3)[nH]n2)on1]

The compound trans-5-(3-(2-(3-methylisoxazol-5-yl) acetamido)-1H-pyrazol-5-yl)tetrahydrofuran-3-yl (1-methylcyclopropyl)carbamate (10 mg, 26 μmol) was purified by PREP-CHIRAL-HPLC (Column: CHIRALPAK IG, 2*25 cm, 5 m; Mobile Phase A: Hex(0.2% DEA)—HPLC, Mobile Phase B: EtOH: DCM=1:1—HPLC; Flow rate: 20 mL/min; Gradient: 80% B to 80% B in 13.5 min; Wave Length: 220/254 nm; RT1(min): 5.27; Sample Solvent: EtOH: DCM=1:1—HPLC; Injection Volume: 1.65 mL; Number Of Runs: 1). Lyophilization yielded (3S,5R)-5-(3-(2-(3-methylisoxazol-5-yl) acetamido)-1H-pyrazol-5-yl)tetrahydrofuran-3-yl (1-methylcyclopropyl)carbamate (4.2 mg, 11 μmol, 80%) as a white amorphous solid.

m/z (ES⁺) [M+H]⁺=390.15; HPLC tR=1.016 min.

¹H NMR (400 MHz, Chloroform-d) 8.85 (s, 1H), 6.58 (s, 1H), 6.19 (s, 1H), 5.38 (s, 1H), 5.20 (d, J=12.7 Hz, 2H), 4.12 (s, 1H), 3.99 (d, J=10.6 Hz, 1H), 3.89 (s, 2H), 2.50 (s, 1H), 2.32 (s, 3H), 2.30-2.22 (m, 1H), 1.39 (s, 3H), 0.79 (s, 2H), 0.68-0.63 (m, 2H).

### (3R,5S)-5-(3-(2-(3-methylisoxazol-5-yl) acetamido)-1H-pyrazol-5-yl) tetrahydrofuran-3-yl (1-methylcyclopropyl) carbamate

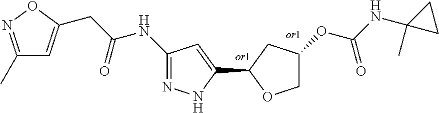 [SMILES: Cc1cc(CC(=O)Nc2cc([C@H]3C[C@H](OC(=O)NC4(C)CC4)CO3)[nH]n2)on1]

The compound trans-5-(3-(2-(3-methylisoxazol-5-yl) acetamido)-1H-pyrazol-5-yl) tetrahydrofuran-3-yl (1-methylcyclopropyl) carbamate (10 mg, 26 μmol) was purified by PREP-CHIRAL-HPLC (Column: CHIRALPAK IG, 2*25 cm, 5 m; Mobile Phase A: Hex(0.2% DEA)—HPLC, Mobile Phase B: EtOH: DCM=1:1—HPLC; Flow rate: 20 mL/min; Gradient: 80% B to 80% B in 13.5 min; Wave Length: 220/254 nm; RT2(min): 10.63; Sample Solvent: EtOH: DCM=1:1—HPLC; Injection Volume: 1.65 mL; Number Of Runs: 1). Lyophilization yielded (3R,5S)-5-(3-(2-(3-methylisoxazol-5-yl) acetamido)-1H-pyrazol-5-yl) tetrahydrofuran-3-yl (1-methylcyclopropyl) carbamate (3.6 mg, 9.2 μmol, 70%) as a white amorphous solid.

m/z (ES⁺) [M+H]⁺=390.15; HPLC tR=1.012 min.

¹H NMR (400 MHz, Chloroform-d) 8.93 (s, 1H), 6.57 (s, 1H), 6.18 (s, 1H), 5.38 (s, 1H), 5.20 (s, 2H), 4.15 (s, 1H), 3.98 (d, J=10.5 Hz, 1H), 3.91 (s, 2H), 2.50 (s, 1H), 2.32 (s, 3H), 2.28-2.24 (m, 1H), 1.39 (s, 3H), 0.80 (s, 2H), 0.73-0.62 (m, 2H).

Additional compounds prepared according to the methods of Example 19 are depicted in Table 10 below.

TABLE 10
Additional Exemplary Compounds

| | | | MS |
|---|---|---|---|
| | | | [M + |
| Compound | Structure | Proton NMR | 1] |
| (3S,5S)-5- (3-(2-(3- methyl- isoxazol- 5-yl) acetamido)- 1H-pyrazol-5- yl)tetrahydro- | 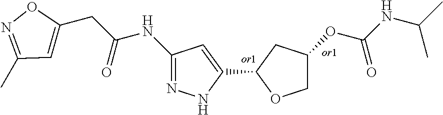 [SMILES: Cc1cc(CC(=O)Nc2cc([C@@H]3C[C@H](OC(=O)NC(C)C)CO3)[nH]n2)on1] | ¹H NMR (400 MHz, DMSO-d₆) 12.37 (s, 1H), 10.69 (s, 1H), 7.08 (d, J = 7.7 Hz, 1H), 6.45 (s, 1H), 6.23 (s, 1H), 5.15 (dq, J = 7.5, 3.5 Hz, 1H), 4.81 (d, J = 7.9 Hz, 1H), 3.84 (d, J = 5.3 Hz, 4H), 3.58 (h, J = 6.7 Hz, 1H), 2.69 (dd, J = 14.6, 6.9 Hz, 1H), 2.20 (s, 3H), 1.90 (t, J = 10.6 Hz, 1H), 1.03 (d, J = 6.6 Hz, 6H). | 378.15 |
| furan-3-yl | | | |
| isopropyl- | | | |
| carbamate | | | |
| (3R,5R)-5- (3-(2-(3- methyl- isoxazol- 5-yl) acetamido)- 1H-pyrazol-5- yl)tetrahydro- | 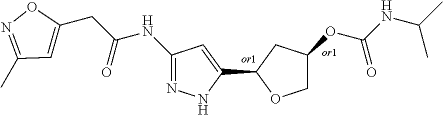 [SMILES: Cc1cc(CC(=O)Nc2cc([C@H]3C[C@@H](OC(=O)NC(C)C)CO3)[nH]n2)on1] | ¹H NMR (400 MHz, DMSO-d₆) 12.37 (s, 1H), 10.69 (s, 1H), 7.08 (d, J = 7.7 Hz, 1H), 6.45 (s, 1H), 6.23 (s, 1H), 5.15 (dq, J = 7.5, 3.6 Hz, 1H), 4.82 (t, J = 7.7 Hz, 1H), 3.84 (d, J = 4.7 Hz, 4H), 3.58 (h, J = 6.7 Hz, 1H), 2.74-2.62 (m, 1H), 2.21 (s, 3H), 1.91 (d, J = 11.9 Hz, 1H), 1.03 (d, J = 6.5 Hz, 6H). | 378.15 |
| furan-3-yl | | | |
| isopropyl- | | | |
| carbamate | | | |
| rel-N-(5- ((2R,4R)- 4-((4-iso- propyl- pyridazin- 3-yl)oxy) tetrahydro- furan-2-yl)- 1H-pyrazol- 3-yl)-2- (3-methyl- | 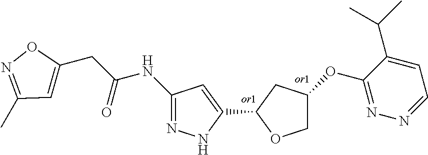 [SMILES: Cc1cc(CC(=O)Nc2cc([C@@H]3C[C@H](Oc4nnccc4C(C)C)CO3)[nH]n2)on1] | ¹H NMR (400 MHz, DMSO-d₆) 12.39 (s, 1H), 10.67 (s, 1H), 8.77 (d, J = 4.7 Hz, 1H), 7.42 (d, J = 4.7 Hz, 1H), 6.46 (s, 1H), 6.22 (s, 1H), 5.78 (s, 1H), 5.06 (s, 1H), 4.14-4.07 (m, 1H), 4.04 (d, J = 10.3 Hz, 1H), 3.83 (s, 2H), 2.94-2.75 (m, 3H), 2.20 (s, 3H), 1.07 (d, J = 6.9 Hz, 6H). | 413.15 |
| isoxazol-5- | | | |
| yl)acetamide | | | |
| rel-N-(5- ((2R,4R)- 4-((4- isopropyl- pyridazin- 3-yl)oxy) tetrahydro- furan-2-yl)- 1H-pyrazol- 3-yl)-2- | 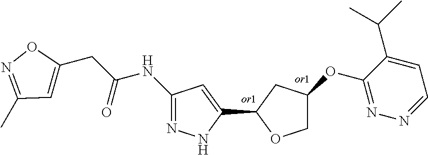 [SMILES: Cc1cc(CC(=O)Nc2cc([C@H]3C[C@@H](Oc4nnccc4C(C)C)CO3)[nH]n2)on1] | ¹H NMR (400 MHz, DMSO-d₆) 12.39 (s, 1H), 10.67 (s, 1H), 8.77 (d, J = 4.8 Hz, 1H), 7.42 (d, J = 4.7 Hz, 1H), 6.46 (s, 1H), 6.22 (s, 1H), 5.78 (s, 1H), 5.06 (s, 1H), 4.15-4.07 (m, 1H), 4.04 (d, J = 10.3 Hz, 1H), 3.83 (s, 2H), 2.94-2.75 (m, 3H), 2.20 (s, 3H), 1.07 (d, J = 6.9 Hz, 6H). | 413.15 |
| (3-methyl- | | | |
| isoxazol- | | | |
| 5-yl)acetamide | | | |
| 2-(3-methyl- isoxazol- 5-yl)-N- (5-(pyrrolidin- 2-yl)-1H- pyrazol- 3-yl)acetamide | 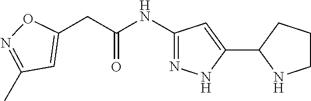 [SMILES: Cc1cc(CC(=O)Nc2cc(C3CCCN3)[nH]n2)on1] | ¹H NMR (400 MHz, Chloroform-d) 10.17 (s, 1H), 6.55-6.51 (m, 1H), 6.14 (s, 1H), 4.31 (s, 1H), 3.83 (s, 2H), 3.12 (s, 1H), 3.03 (s, 1H), 2.29 (s, 5H), 1.89 (s, 3H). | 276.20 |
| rel-N-(5- ((2R,4R)-4- ((4-isopropyl- pyridazin- 3-yl)oxy)tetra- hydrofuran-2- yl)-1H- pyrazol- 3-yl)-3- (methoxy- methyl)- 1-methyl-1H- pyrazole-5- | 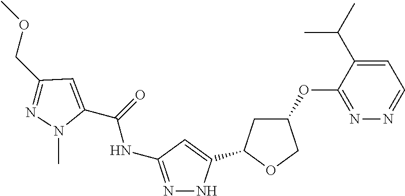 [SMILES: COCc1cc(C(=O)Nc2cc([C@@H]3C[C@H](Oc4nnccc4C(C)C)CO3)[nH]n2)n(C)n1] | ¹H NMR (400 MHz, DMSO-d₆) 12.52 (s, 1H), 10.80 (s, 1H), 8.78 (d, J = 4.7 Hz, 1H), 7.44 (dd, J = 4.8, 0.8 Hz, 1H), 7.13 (s, 1H), 6.61 (s, 1H), 5.81 (s, 1H), 5.10 (s, 1H), 4.34 (s, 2H), 4.18-4.07 (m, 2H), 4.06 (s, 3H), 2.98 (p, J = 7.0 Hz, 1H), 2.84 (dt, J = 14.4, 7.4 Hz, 1H), 2.35-2.26 (m, 1H), 1.11 (dd, J = 6.9, 3.0 Hz, 6H). | 442.15 |
| carboxamide | | | |
| rel-N-(5- ((2R,4R)- 4-((4- isopropyl- pyridazin- 3-yl)oxy)tetra- hydrofuran-2- yl)-1H- pyrazol- 3-yl)-3- (methoxy- methyl)- 1-methyl-1H- | 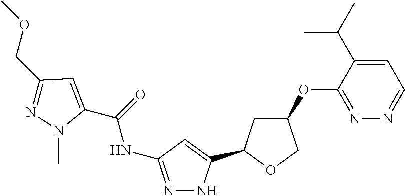 [SMILES: COCc1cc(C(=O)Nc2cc([C@H]3C[C@@H](Oc4nnccc4C(C)C)CO3)[nH]n2)n(C)n1] | ¹H NMR (400 MHz, DMSO-d₆) 12.52 (s, 1H), 10.81 (s, 1H), 8.78 (d, J = 4.8 Hz, 1H), 7.44 (dd, J = 4.8, 0.8 Hz, 1H), 7.13 (s, 1H), 6.61 (s, 1H), 5.81 (s, 1H), 5.10 (s, 1H), 4.34 (s, 2H), 4.17-4.07 (m, 2H), 4.06 (s, 3H), 2.98 (p, J = 6.8 Hz, 1H), 2.84 (dt, J = 14.3, 7.5 Hz, 1H), 2.30 (dd, J = 14.9, 5.4 Hz, 1H), 1.11 (dd, J = 6.9, 2.9 Hz, 6H). | 442.15 |
| pyrazole-5- | | | |
| carboxamide | | | |
| (3S,5S)-5- (5-(2-(3- methyl- isoxazol- 5-yl) acetamido)- 1H-pyrazol-3- yl)tetrahydro- | 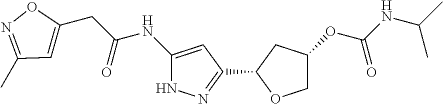 [SMILES: Cc1cc(CC(=O)Nc2cc([C@@H]3C[C@H](OC(=O)NC(C)C)CO3)n[nH]2)on1] | ¹H NMR (400 MHz, DMSO-d₆) 12.38 (s, 1H), 10.69 (s, 1H), 7.09 (d, J = 7.8 Hz, 1H), 6.44 (s, 1H), 6.23 (s, 1H), 5.15 (s, 1H), 4.82 (s, 1H), 3.84 (d, J = 4.9 Hz, 4H), 3.58 (q, J = 6.8 Hz, 1H), 2.67 (s, 1H), 2.21 (s, 3H), 1.90 (s, 1H), 1.03 (d, J = 6.5 Hz, 6H). | 378.15 |
| furan-3-yl | | | |
| isopropyl- | | | |
| carbamate | | | |
| rel-N-(5- ((2R,4R)- 4-((4- cyclopropyl- pyridin-3- yl)oxy)tetra- hydrofuran-2- yl)-1H- pyrazol- 3-yl)-3- (methoxy- methyl)- 1-methyl-1H- pyrazole-5- carboxamide | 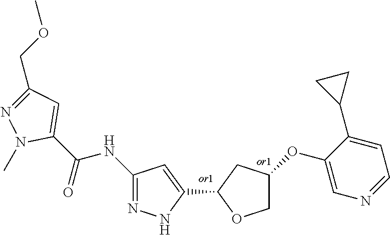 [SMILES: COCc1cc(C(=O)Nc2cc([C@@H]3C[C@H](Oc4cnccc4C4CC4)CO3)[nH]n2)n(C)n1] | ¹H NMR (400 MHz, Chloroform-d) 9.27 (s, 1H), 8.23-7.99 (m, 2H), 6.85-6.71 (m, 2H), 6.69 (d, J = 5.0 Hz, 1H), 5.33-5.03 (m, 2H), 4.50 (d, J = 1.5 Hz, 2H), 4.33 (d, J = 10.3 Hz, 1H), 4.28-4.04 (m, 4H), 3.46 (d, J = 1.5 Hz, 3H), 2.82 (dt, J = 14.7, 7.5 Hz, 1H), 2.62-2.38 (m, 1H), 2.03 (d, J = 13.4 Hz, 1H), 1.05 (dd, J = 8.4, 2.2 Hz, 2H), 0.78-0.63 (m, 2H). | 439.20 |
| rel-N-(5- ((2R,4R)- 4-((4- cyclopro- pylpyridin-3- yl)oxy)tetra- hydrofuran-2- yl)-1H- pyrazol- 3-yl)-3- (methoxy- methyl)- 1-methyl-1H- pyrazole-5- carboxamide | 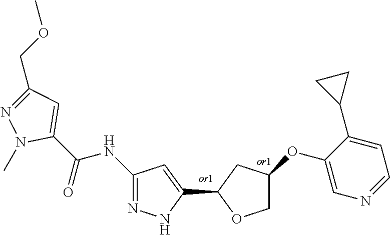 [SMILES: COCc1cc(C(=O)Nc2cc([C@H]3C[C@@H](Oc4cnccc4C4CC4)CO3)[nH]n2)n(C)n1] | ¹H NMR (400 MHz, Chloroform-d) 9.35 (d, J = 16.1 Hz, 1H), 8.13 (d, J = 20.1 Hz, 2H), 6.83-6.74 (m, 2H), 6.69 (d, J = 4.9 Hz, 1H), 5.31-5.11 (m, 2H), 4.50 (d, J = 2.2 Hz, 2H), 4.32 (d, J = 10.4 Hz, 1H), 4.26-4.00 (m, 4H), 3.46 (d, J = 2.3 Hz, 3H), 2.92-2.74 (m, 1H), 2.61-2.37 (m, 1H), 2.05 (s, 1H), 1.04 (ddd, J = 10.8, 5.4, 3.2 Hz, 2H), 0.71 (dd, J = 5.5, 2.8 Hz, 2H). | 439.15 |

### Example 20

### rel-(1R,3S)-3-(2-(2-(3-methylisoxazol-5-yl)acetamido)thiazol-5-yl)cyclopentyl isopropylcarbamate and

### rel-(1R,3S)-3-(2-(2-(3-methylisoxazol-5-yl)acetamido)thiazol-5-yl)cyclopentyl isopropylcarbamate

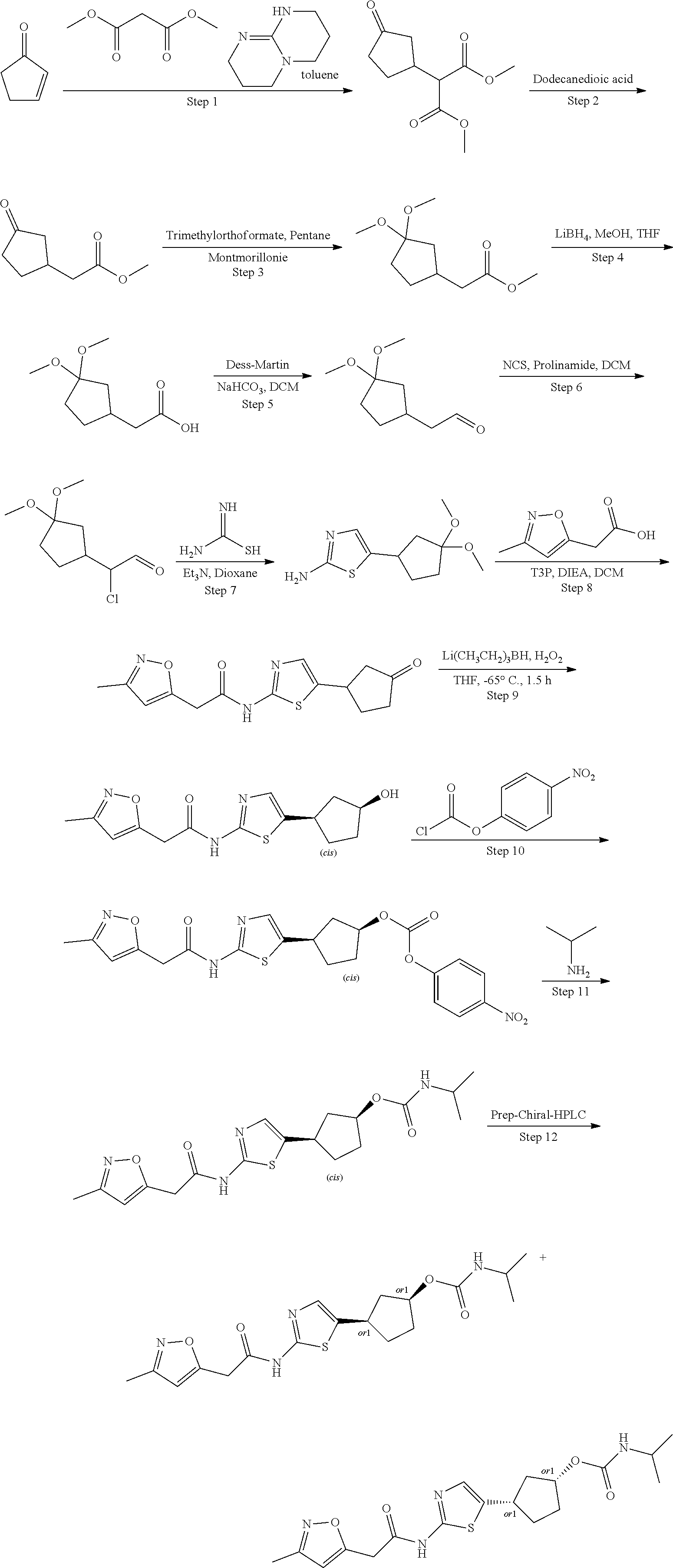

### dimethyl 2-(3-oxocyclopentyl)malonate

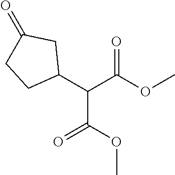

Step 1: To a solution of 2-cyclopenten-1-one (25.5 mL, 305 mmol) in dry toluene (250 mL) was added dimethyl malonate (140 mL, 1.22 mol). 1,5,7-triazabicyclo[4.4.0]dec-5-ene (1.27 g, 9.14 mmol) was then added and the mixture was stirred for 20 h at room temperature under nitrogen. The mixture was concentrated under reduced pressure to ½ volume. The crude product was filtered through a short pad of silica gel (4 cm×4 cm), eluting with AcOEt several times. The filtrate was concentrated under reduced pressure (70-75° C. water bath, 2 h) to afford the title compound as a yellow oil (86 g).

LCMS: Not MS sensitive.

¹H NMR (400 MHz, DMSO): δ 3.67 (s; 3 H); 3.65 (s; 3 H); 3.52 (s; 1 H); 2.65-2.76 (m; 1 H); 2.29 (dd;J=18.10; 7.54 Hz; 1H); 2.15-2.19 (m; 2 H); 1.96-2.05 (m; 2 H); 1.53-1.64 (m; 1 H).

### methyl 2-(3-oxocyclopentyl)acetate

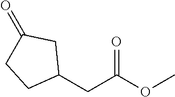

Step 2: A mixture of methyl 3-(dimethyl-13-oxidaneyl)-3-oxo-2-(3-oxocyclopentyl)propanoate (40.0 g, 187 mmol) and dodecanedioic acid (34.4 g, 149 mmol) was heated at 210° C. (metallic bille temperature) for 20 h. The mixture was then distilled by using a fractionating column under reduced pressure (vaccum: 8-10 mbar, metallic bille temperature:180-190° C., interior temperature: 70-100° C., used distillation receiver) to afford the title compound as an oil transparent (17.1 g, 59%).

¹H NMR (400 MHz, CDCl₃): δ 3.68 (s; 3 H); 2.55-2.67 (m; 1 H); 2.43-2.51 (m; 3 H); 2.26-2.35 (m; 1 H); 2.14-2.25 (m; 2 H); 1.88 (dd; J=18.23; 10.09 Hz; 1 H); 1.51-1.62 (m; 1 H).

### methyl 2-(3,3-dimethoxycyclopentyl)acetate

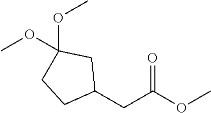

Step 3: A mixture of montmorillonite (21.0 g, 64.0 mmol) and trimethylorthoformate (35.0 mL, 320 mmol) was stirred at room temperature for 2 h. A solution of methyl 2-(3-oxocyclopentyl)acetate (10.0 g, 64.0 mmol) in pentane (48 mL) was then added slowly over 10 minutes and the mixture was stirred at room temperature for 20 h. The mixture was then filtered over a short pad of celite and rinsed with pentane. The filtrate was concentrated under reduced pressure to afford the title compound as an oil transparent (12.7 g, 98%).

¹H NMR (400 MHz, CDCl₃): δ 3.65 (s; 3 H); 3.18 (s; 6 H); 2.30-2.44 (m; 3 H); 2.08 (dd; J=13.22; 7.51 Hz; 1 H); 1.84-1.92 (m; 2 H); 1.72-1.80 (m; 1 H); 1.42 (dd; J=13.18; 8.58 Hz; 1H); 1.27-1.37 (m; 1 H).

### 2-(3,3-dimethoxycyclopentyl)ethan-1-ol

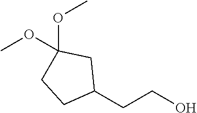

Step 4: A solution of methyl 2-(3,3-dimethoxycyclopentyl)acetate (3.00 g, 14.8 mmol) in THE (15.0 mL) was treated by a 2 M solution of lithium borohydride in THE (8.90 mL, 17.8 mmol) dropwise at room temperature. Methanol (720 uL, 17.8 mmol) was then added dropwise and the mixture was stirred at room temperature for 20 h. A saturated solution of NH₄Cl (30 mL) was then added and the mixture was diluted with ether (50 mL). Layers were then separated and organic phase was extracted with ether (30 mL, 2X). The combined organic phases were then washed with brine, dried over Na₂SO₄, filtered and concentrated under reduced pressure. The crude compound was purified over a 40 g silica gel column using a mixture of EtOAc in hexane gradient (0-50%) using a ELSD dectector, to afford the title compound as an oil transparent (1.8 g, 70%).

¹H NMR (400 MHz, CDCl₃): δ 3.65 (q; J=5.92 Hz; 2 H); 3.19 (d; J=3.99 Hz; 6 H); 2.02-2.13 (m; 2 H); 1.84-1.90 (m; 2 H); 1.72-1.79 (m; 1 H); 1.58-1.65 (m; 2 H); 1.27-1.42 (m; 3 H).

### 2-(3,3-dimethoxycyclopentyl)acetaldehyde

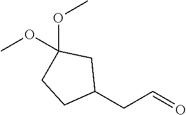

Step 5: To a solution of 2-(3,3-dimethoxycyclopentyl)ethan-1-ol (800 mg, 4.59 mmol) in CH₂Cl2 (50 mL) was added sodium bicarbonate (1.54 g, 18.4 mmol) and the mixture was stirred for 5 minutes. The mixture was cooled to 0° C. and Dess-Martin Periodinane (2.92 g, 6.89 mmol) was added in portion. The mixture stirred for 30 minutes at 0° C. and then the ice bath was removed and the mixture was stirred for 2 h at rt. A mixture of a saturated solution of NaHCO₃(30 mL) and Na₂S204 (30 mL) were added and the mixture was stirred for 30 minutes. The mixture was then diluted in DCM (70 mL). A queuse phase was extracted with DCM (30 mL, 2X). The combined organic phases were washed with brine, dried over Na₂SO₄, filtered and concentrated under reduced pressure to afford the title compound as an oil (890 mg).

¹H NMR (400 MHz, CDCl₃): δ 9.75 (s; 1 H); 3.19 (s; 6 H); 2.49 (s; 3 H); 2.06-2.15 (m; 1 H); 1.85-1.94 (m; 2 H); 1.74-1.82 (m; 1 H); 1.30-1.43 (m; 2 H).

### 2-chloro-2-(3,3-dimethoxycyclopentyl)acetaldehyde

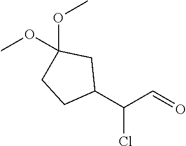

Step 6: To a solution of the crude of 2-(3,3-dimethoxycyclopentyl)acetaldehyde (1.00 g, 5.23 mmol), 90% purity, in CH₂Cl2 (45 mL) was added L-(−)-prolinamide (119 mg, 1.05 mmol), and the mixture was stirred for 10 minutes at 0° C.. A solution of N-chlorosuccinimide (698 mg, 5.23 mmol) in CH₂Cl2 (20 mL) was added drop by drop over 5 minutes at 0° C. and the mixture was then stirred at 0° C. for 1 h. The ice bath was then removed and the mixture was stirred for 9 h at room temperature. The mixture was cooled to 0° C. and pentane (40 mL) was added and the mixture was stirred for 10 minutes at 0° C. The mixture was then filtered over celite and then concentrated under reduced pressure. The residue was diluted in pentane (50 mL) and was washed with brine (10 mL, 2X), dried over Na₂SO₄, filtered, and concentrated under reduced pressure to afford the crude of the title compound as an oil transparent (670 mg, 62%).

¹H NMR (400 MHz, CDCl₃): δ 9.45 (dd; J=6.67; 2.89 Hz; 1 H); 4.13 (td; J=7.88; 2.85 Hz; 1 H); 3.20 (s; 6 H); 2.58-2.64 (m; 1 H); 1.99-2.10 (m; 1 H); 1.75-1.94 (m; 4 H); 1.62-1.69 (m; 1 H).

### 5-(3,3-dimethoxycyclopentyl)thiazol-2-amine

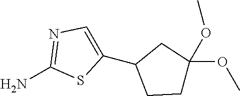

Step 7: A suspension of thiourea (494 mg, 6.48 mmol) in dioxane (10 mL) was sonicated, then triethylamine (3.16 mL, 22.7 mmol) was added at room temperature, and the mixture was stirred for 10 minutes. A solution of 2-bromo-2-(3,3-dimethoxycyclopentyl)acetaldehyde (670 mg, 3.24 mmol) in dioxane (17 mL) was then added and the mixture was quickly transferred to a preheated heating bath set at 80° C. and was heated for 20 h. The mixture cooled to room temperature and concentrated under reduced pressure. The residue was diluted in ether (70 mL) and was washed with semi-saturated brine (10 mL, 2X). The aqueous phase was then extracted with ether (30 mL, 2X). The combined organic phases were dried over Na₂SO₄, filtered, and concentrated under reduced pressure. The residue was purified over a silica gel column using a mixture of 10% MeOH in DCM gradient to afford the title compound as a brown oil (200 mg, 27%).

¹H NMR (400 MHz, CDCl₃): δ 6.76 (s; 1 H); 4.76 (bs; 2 H); 3.26-3.30 (m; 1 H); 3.22 (d; J=3.47 Hz; 6H); 2.30 (dd; J=13.18; 7.68 Hz; 1 H); 2.06-2.13 (m; 1 H); 1.96-2.02 (m; 1 H); 1.84-1.90 (m; 1 H); 1.69-1.80 (m; 2 H). ESI-MS (m/z+): 229.1 [M+H].

### N-(5-(3,3-dimethoxycyclopentyl)thiazol-2-yl)-2-(3-methylisoxazol-5-yl)acetamide

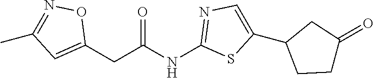 [SMILES: Cc1cc(CC(=O)Nc2ncc(C3CCC(=O)C3)s2)on1]

Step 8: To a solution of 5-(3,3-dimethoxycyclopentyl)thiazol-2-amine (480 mg, 2.10 mmol) in CH₂Cl2 (30 mL) was added 3-methyl-5-isoxazoleacetic acid (333 mg, 2.31 mmol) at 0° C. N,N-diisopropylethylamine (1.10 mL, 6.31 mmol) was then added followed by a 50% solution of propyl phosphonic anhydride (3.75 mL, 6.31 mmol) in EtOAc, and the mixture was slowly warmed to room temperature (over 4 hours). The mixture was then diluted in DCM (30 mL), and a saturated solution of brine (10 mL) was added. Phases were separated, and aqeous phase was extracted with DCM (20 mL, 2X). Combined organic phases were dried over Na₂SO₄, filtered, and concentrated under reduced pressure. The residue was purified over a silica gel column using a mixture of EtOAc in DDM graident (0-60%) to afford the title compound as a yellow-pale solid (270 mg, 42%).

¹HNMR (400 MHz, CDCl₃): δ 10.68 (br s; 1 H); 7.19 (s; 1 H); 6.19 (s; 1 H); 3.97 (s; 2 H); 3.56-3.65 (m; 1 H); 2.73 (dd; J=18.07; 7.52 Hz; 1 H); 2.44-2.56 (m; 2 H); 2.28-2.37(m; 2 H); 2.31 (s, 3H), 2.00-2.10 (m; 1 H). ESI-MS (m/z+): 306.1[M+H].

### N-(5-((1R,3S)-3-hydroxycyclopentyl)thiazol-2-yl)-2-(3-methylisoxazol-5-yl)acetamide

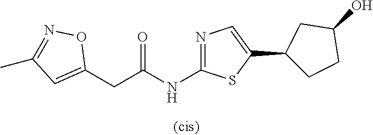 [SMILES: Cc1cc(CC(=O)Nc2ncc([C@@H]3CC[C@H](O)C3)s2)on1]

Step 9: To a mixture of 2-(3-methylisoxazol-5-yl)-N-(5-(3-oxocyclopentyl)thiazol-2-yl)acetamide (250 mg, 1 Eq, 819 μmol) in THF (10 mL) was added Li(CH₃CH₂)₃BH (1.64 mL, 1 molar, 2 Eq, 1.64 mmol) drop wise at −65° C. under nitrogen atmosphere. The mixture was stirred for 1 h at −65° C. The mixture was quenched with NaHCO₃(aq.) at −65° C., then added H₂O₂ at −10° C. and stirred for 1 h. The reaction mixture was diluted with water (15 mL), and the aqueous phase was extracted with EA (10 mL) three times. The combined organic layers were washed with brine, dried over sodium sulfate, filtered, and concentrated in vacuo. This resulted in N-(5-(3-hydroxycyclopentyl)thiazol-2-yl)-2-(3-methylisoxazol-5-yl)acetamide (200 mg, 651 μmol, 79.5%) as a yellow amorphous solid. The resulting crude material was purified by Pre-HPLC (Column: XBridge Shield R^{P}18 OBD Column, 30*150 mm, 5 m; Mobile Phase A:Water(10 mmol/L NH₄HCO₃+0.1%NH3·H₂O), Mobile Phase B: ACN; Flow rate: 60 mL/min; Gradient: 25% B to 50% B in 8 min, 50% B; Wave Length: 220 nm; RT1(min): 7.48; Number Of Runs: 0). Lyophilization yielded N-(5-((cis)-3-hydroxycyclopentyl)thiazol-2-yl)-2-(3-methylisoxazol-5-yl)acetamide (150 mg, 488 μmol, 75.0%) as an off-white amorphous solid.

m/z (ES⁺) [M+H]⁺=308.0; HPLC tR=0.630 min.

### (cis)-3-(2-(2-(3-methylisoxazol-5-yl)acetamido)thiazol-5-yl)cyclopentyl (4-nitrophenyl) carbonate

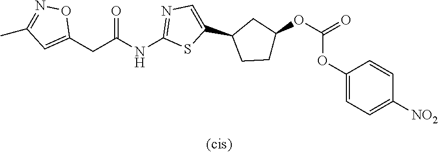 [SMILES: Cc1cc(CC(=O)Nc2ncc([C@@H]3CC[C@H](OC(=O)Oc4ccc([N+](=O)[O-])cc4)C3)s2)on1]

Step 10: A round bottomed flask was charged with N-(5-((cis)-3-hydroxycyclopentyl)thiazol-2-yl)-2-(3-methylisoxazol-5-yl)acetamide (145 mg, 1Eq, 472 μmol), DCM (6 mL),4-nitrophenyl carbonochloridate (95.1 mg, 1 Eq, 472 μmol), Py (112 mg, 114 L, 3 Eq, 1.42 mmol), DMAP (5.76 mg, 0.1 Eq, 47.2 μmol) and a stirbar, and the solution was stirred for 16 hour at 25° C. The reaction mixture was concentrated in vacuo resulting in (cis)-3-(2-(2-(3-methylisoxazol-5-yl)acetamido)thiazol-5-yl)cyclopentyl (4-nitrophenyl) carbonate (250 mg, crude) as an off-white amorphous solid used in next step without further purification.

m/z (ES⁺) [M+H]⁺=473.0; HPLC tR=0.978 min.

### (cis)-3-(2-(2-(3-methylisoxazol-5-yl)acetamido)thiazol-5-yl)cyclopentyl isopropylcarbamate

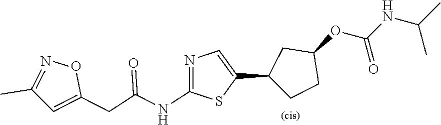 [SMILES: Cc1cc(CC(=O)Nc2ncc([C@@H]3CC[C@H](OC(=O)NC(C)C)C3)s2)on1]

Step 11: A round bottomed flask was charged with (cis)-3-(2-(2-(3-methylisoxazol-5-yl)acetamido)thiazol-5-yl)cyclopentyl (4-nitrophenyl)carbonate (135 mg, 1 Eq, 286 μmol), 2-MeTHF (5 mL), DIEA (73.9 mg, 99.5 L, 2 Eq, 571 μmol), propan-2-amine (33.8 mg, 2 Eq, 571 μmol) and a stirbar, and the solution was stirred for 16 hour at 25° C. The reaction mixture was diluted with water (10 mL), and the aqueous phase was extracted with EA (10 mL) three times. The combined organic layers were washed with brine, dried over sodium sulfate, filtered, and concentrated in vacuo. The resulting crude material was purified by Pre-HPLC (Column: XBridge Shield R^{P}18 OBD Column, 30*150 mm, 5 m; Mobile Phase A: Water(10 mmol/L NH₄HCO₃+0.1% NH₃·H₂O), Mobile Phase B: ACN; Flow rate: 60 mL/min; Gradient: 25% B to 50% B in 8 min, 50% B; Wave Length: 220 nm; RT1(min): 7.48; Number Of Runs: 0). Lyophilization yielded (cis)-3-(2-(2-(3-methylisoxazol-5-yl)acetamido)thiazol-5-yl)cyclopentyl isopropylcarbamate (80 mg, 0.20 mmol, 71%) as an off-white amorphous solid.

m/z (ES⁺) [M+H]⁺=393.2; HPLC tR=0.978 min.

### rel-(1R,3S)-3-(2-(2-(3-methylisoxazol-5-yl)acetamido)thiazol-5-yl)cyclopentyl isopropylcarbamate

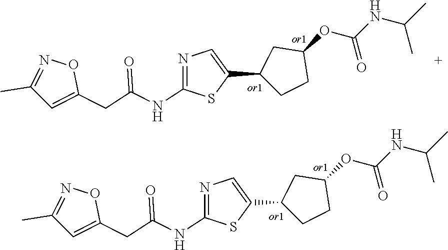

Step 12: The resulting material (cis)-3-(2-(2-(3-methylisoxazol-5-yl)acetamido)thiazol-5-yl)cyclopentyl isopropylcarbamate (80 mg, 0.20 mmol) was purified by chiral Pre-HPLC (Column: CHIRALPAK IG, 2*25 cm, 5 m; Mobile Phase A: Hex(0.2% DEA)—HPLC, Mobile Phase B: MeOH: DCM=1:1; Flow rate: 20 mL/min; Gradient: 50% B to 50% B in 11 min; Wave Length: 220/254 nm; RT1(min): 7.17; RT2(min): 8.83; Sample Solvent: MeOH: DCM=1:1; Injection Volume: 0.5 mL; Number Of Runs: 5). Lyophilization yielded rel-(1R,3S)-3-(2-(2-(3-methylisoxazol-5-yl)acetamido)thiazol-5-yl)cyclopentyl isopropylcarbamate (32 mg, 82 μmol, 40%) as an off-white amorphous solid.

m/z (ES⁺) [M+H]⁺=393.10; HPLC tR=0.797 min.

¹H NMR (400 MHz, DMSO-d₆) 12.28 (s, 1H), 7.22 (d, J=1.0 Hz, 1H), 6.96 (d, J=7.9 Hz, 1H), 6.28 (s, 1H), 5.00 (s, 1H), 4.00 (s, 2H), 3.58 (h, J=6.7 Hz, 1H), 3.32-3.22 (m, 1H), 2.50 (s, 1H), 2.21 (s, 3H), 2.05 (d, J=9.9 Hz, 1H), 1.91 (s, 1H), 1.70 (dd, J=20.7, 10.5 Hz, 2H), 1.58 (s, 1H), 1.03 (d, J=6.6 Hz, 6H).

### Example 21

### ((1s,3s)-3-(3-(2-(3-methylisoxazol-5-yl)acetamido)-1H-pyrazol-5-yl)cyclobutyl)methyl isopropylcarbamate and

### ((1s,3s)-3-(3-(2-(3-methylisoxazol-5-yl)acetamido)-1H-pyrazol-5-yl)cyclobutyl)methyl isopropylcarbamate

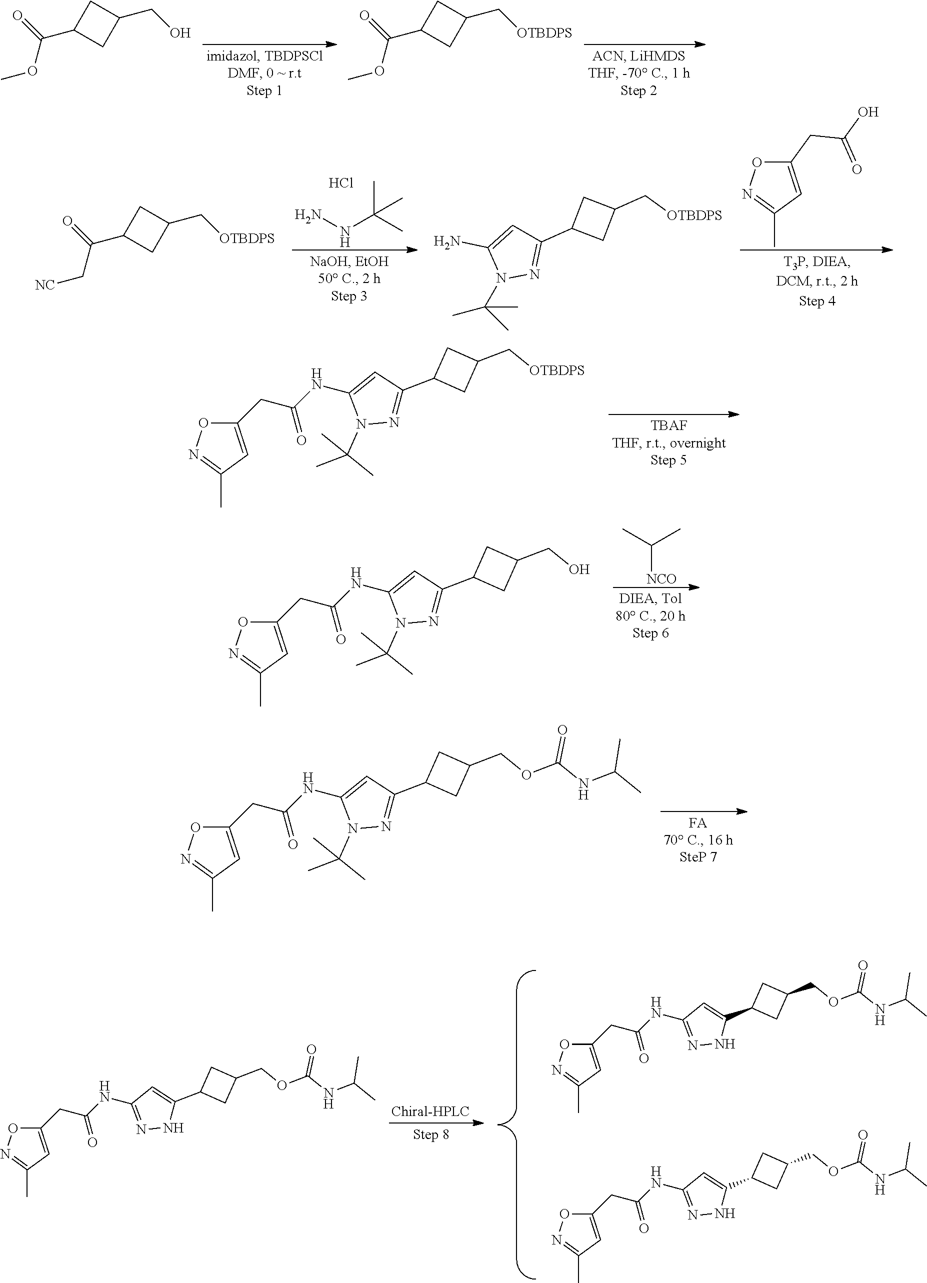

### methyl 3-(((tert-butyldiphenylsilyl)oxy)methyl)cyclobutane-1-carboxylate

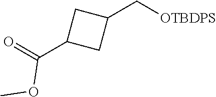

Step 1: A round bottomed flask was charged with methyl 3-(hydroxymethyl)cyclobutane-1-carboxylate (2.5 g, 1 Eq, 17 mmol), imidazole (3.5 g, 3 Eq, 52 mmol), DMF (10 mL) and a stirbar. Tert-butyldiphenylsilyl hypochlorite (12 g, 2.5 Eq, 43 mmol) was added, and the solution was stirred at 25° C. for 16 hours. The mixture was diluted with water, and the aqueous phase was extracted with EA three times. The combined organic layers were washed with brine, dried over sodium sulfate, filtered, and concentrated in vacuo. This resulted in methyl 3-(((tert-butyldiphenylsilyl)oxy)methyl)cyclobutane-1-carboxylate (5 g, crude) as a yellow oil.

m/z (ES⁺) [M+H]⁺=383.30; HPLC tR=1.143 min.

### 3-(3-(((tert-butyldiphenylsilyl)oxy)methyl)cyclobutyl)-3-oxopropanenitrile

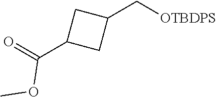

Step 2: To a solution of methyl 3-(((tert-butyldiphenylsilyl)oxy)methyl)cyclobutane-1-carboxylate (5 g, 1 Eq, 0.01 mol) in THE (30 mL), CH₃CN (1 g, 1 mL, 2 Eq, 0.03 mol) was added. Then bubbling nitrogen through the reaction mixture for 2 minutes and cooled to −78° C., LiHMDS (3 g, 20 mL, 1.5 Eq, 0.02 mol) was added dropwise to the reaction. The mixture was stirred at −78° C. for 1 h. The mixture was evaporated and extracted with ethyl acetate (3×40 ml), dried over Na₂SO₄ and evaporated in vacuo. The crude residue was purified by flash (Mobile Phase A: Water, Mobile Phase B: ACN; Flow rate: 60 mL/min; Gradient: 60% B to 80% B in 7 min); After solvent evaporation afforded the title compound 3-(3-(((tert-butyldiphenylsilyl)oxy)methyl)cyclobutyl)-3-oxopropanenitrile (2.68 g, 6.84 mmol, 50%) as a yellow oil.

m/z (ES⁺) [M+Na]=414.10; HPLC tR=1.425 min.

### 1-(tert-butyl)-5-(3-(((tert-butyldiphenylsilyl)oxy)methyl)cyclobutyl)-1H-pyrazol-3-amine

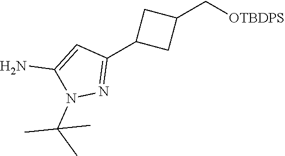

Step 3: A round bottomed flask was charged with tert-butylhydrazine hydrochloride (1.28 g, 1.5 Eq, 10.3 mmol), and NaOH (0.27 g, 1 Eq, 6.84 mmol) in EtOH (13 mL) and a stirbar. The resulting mixture was stirred for 1-2 hours. Then 3-(3-(((tert-butyldiphenylsilyl)oxy)methyl)cyclobutyl)-3-oxopropanenitrile (2.68 g, 1 Eq, 6.84 mmol) was dissolved with EtOH (5 mL), and the above mixture was added. The resulting solution was stirred at 50° C. for 2 hours. The resulting crude material was purified by Flash (acetonitrile/water/0.1% formic acid) and concentrated in vacuo to afford 1-(tert-butyl)-5-(3-(((tert-butyldiphenylsilyl)oxy)methyl)cyclobutyl)-1H-pyrazol-3-amine (1.98 g, 4.29 mmol, 62.7%) as a brown oil.

m/z (ES⁺) [M+H]⁺=462.25; HPLC tR=1.599 min.

### N-(1-(tert-butyl)-5-(3-(((tert-butyldiphenylsilyl)oxy)methyl)cyclobutyl)-1H-pyrazol-3-yl)-2-(3-methylisoxazol-5-yl)acetamide

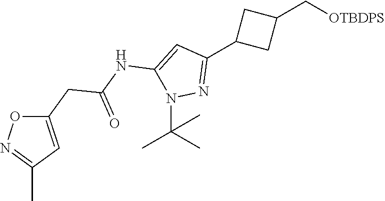

Step 4: A round bottomed flask was charged with 1-(tert-butyl)-5-(3-(((tert-butyldiphenylsilyl)oxy)methyl)cyclobutyl)-1H-pyrazol-3-amine (1.98 g, 1 Eq, 4.29 mmol), DCM (10 mL) and a stirbar, 2-(3-methylisoxazol-5-yl)acetic acid (908 mg, 1.5 Eq, 6.43 mmol), DIEA (1.66 g, 3 Eq, 12.9 mmol), T3P (5.45 g, 50% Wt, 2 Eq, 8.58 mmol) were added. The solution was stirred at 25° C. for 2 hours. The solution was quenched with water, and the organic phase was collected. The aqueous phase was extracted with EA three times. The organic phase was combined and concentrated. The resulting crude material was purified by Flash (acetonitrile/water) and concentrated in vacuo to afford N-(1-(tert-butyl)-5-(3-(((tert-butyldiphenylsilyl)oxy)methyl)cyclobutyl)-1H-pyrazol-3-yl)-2-(3-methylisoxazol-5-yl)acetamide (2.50 g, 4.27 mmol, 99.7%) as a brown amorphous solid.

m/z (ES⁺) [M+H]⁺=585.40; HPLC tR=1.575 min.

### N-(1-(tert-butyl)-5-(3-(hydroxymethyl)cyclobutyl)-1H-pyrazol-3-yl)-2-(3-methylisoxazol-5-yl)acetamide

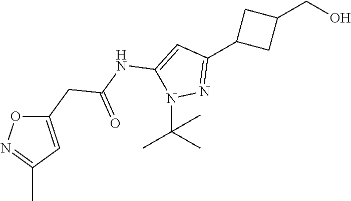 [SMILES: Cc1cc(CC(=O)Nc2cc(C3CC(CO)C3)nn2C(C)(C)C)on1]

Step 5: A round bottomed flask was charged with N-(1-(tert-butyl)-5-(3-(((tert-butyldiphenylsilyl)oxy)methyl)cyclobutyl)-1H-pyrazol-3-yl)-2-(3-methylisoxazol-5-yl)acetamide (2.5 g, 1 Eq, 4.3 mmol), THE (15 mL) and a stirbar. TBAF (2.2 g, 8.5 mL, 1 molar, 2 Eq, 8.5 mmol) was added, and the solution was stirred at 25° C. for 2 days. The mixture was diluted with water, and the aqueous phase was extracted with EA three times. The combined organic layers were washed with brine, dried over sodium sulfate, filtered, and concentrated in vacuo. The resulting crude material was purified by Flash (MeCN/NH₃·H₂O in water) and concentrated in vacuo to afford N-(1-(tert-butyl)-5-(3-(hydroxymethyl)cyclobutyl)-1H-pyrazol-3-yl)-2-(3-methylisoxazol-5-yl)acetamide (900 mg, 2.60 mmol, 61%) as a brown amorphous solid.

m/z (ES⁺) [M+H]⁺=347.20; HPLC tR=0.800 min.

### (3-(1-(tert-butyl)-3-(2-(3-methylisoxazol-5-yl)acetamido)-1H-pyrazol-5-yl)cyclobutyl)methyl isopropylcarbamate

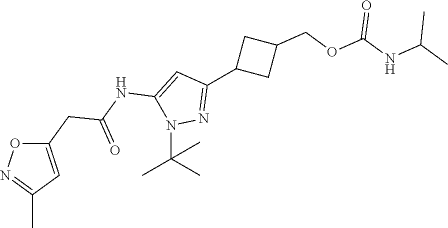 [SMILES: Cc1cc(CC(=O)Nc2cc(C3CC(COC(=O)NC(C)C)C3)nn2C(C)(C)C)on1]

Step 6: A round bottomed flask was charged with N-(1-(tert-butyl)-5-(3-(hydroxymethyl)cyclobutyl)-1H-pyrazol-3-yl)-2-(3-methylisoxazol-5-yl)acetamide (450 mg, 1 Eq, 1.30 mmol), DIEA (504 mg, 679 μL, 3 Eq, 3.90 mmol), 2-isocyanatopropane (221 mg, 2 Eq, 2.60 mmol), Tol (15 mL) and a stirbar. The solution was stirred at 80° C. for 20 hours and concentrated in vacuo. The resulting crude material was purified by Flash (acetonitrile/water) and concentrated in vacuo resulting in (3-(1-(tert-butyl)-3-(2-(3-methylisoxazol-5-yl)acetamido)-1H-pyrazol-5-yl)cyclobutyl)methyl isopropylcarbamate (320 mg, 742 μmol, 57.1%) as a yellow oil.

m/z (ES⁺) [M+H]⁺=432.35; HPLC tR=0.835 min.

### (3-(3-(2-(3-methylisoxazol-5-yl)acetamido)-1H-pyrazol-5-yl)cyclobutyl)methyl isopropylcarbamate

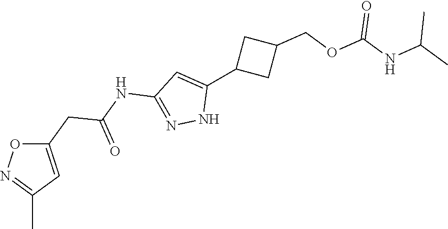 [SMILES: Cc1cc(CC(=O)Nc2cc(C3CC(COC(=O)NC(C)C)C3)[nH]n2)on1]

Step 7: A round bottomed flask was charged with (3-(1-(tert-butyl)-3-(2-(3-methylisoxazol-5-yl)acetamido)-1H-pyrazol-5-yl)cyclobutyl)methyl isopropylcarbamate (320 mg, 1 Eq, 742 μmol), FA (15 mL) and a stirbar, and the solution was stirred at 70° C. for 16 hours. The resulting mixture was concentrated in vacuo resulting in (3-(3-(2-(3-methylisoxazol-5-yl)acetamido)-1H-pyrazol-5-yl)cyclobutyl)methyl isopropylcarbamate (330 mg, 0.66 mmol, 89%, 75% Purity) as a yellow oil.

m/z (ES⁺) [M+H]⁺=376.05; HPLC tR=0.775 min.

### ((1s,3s)-3-(3-(2-(3-methylisoxazol-5-yl)acetamido)-1H-pyrazol-5-yl)cyclobutyl)methyl isopropylcarbamate

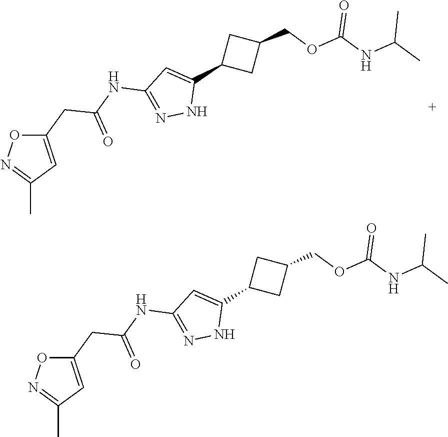 [SMILES: Cc1cc(CC(=O)Nc2cc([C@H]3C[C@@H](COC(=O)NC(C)C)C3)[nH]n2)on1 ; Cc1cc(CC(=O)Nc2cc([C@H]3C[C@@H](COC(=O)NC(C)C)C3)[nH]n2)on1]

Step 8: (3-(3-(2-(3-methylisoxazol-5-yl)acetamido)-1H-pyrazol-5-yl)cyclobutyl)methyl isopropylcarbamate (250 mg, 1 Eq, 666 μmol) was purified by chiral prep-HPLC (Column: CHIRALPAK IG, 2*25 cm, 5 m; Mobile Phase A: Hex(0.2% DEA)—HPLC, Mobile Phase B: MeOH: DCM=1:1; Flow rate: 20 mL/min; Gradient: 80% B to 80% B in 10 min; Wave Length: 220/254 nm; RT1(min): 4.18; RT2(min): 7.47; Sample Solvent: MeOH: DCM=1:1; Injection Volume: 1 mL; Number Of Runs: 3). Lyophilization yielded ((1s,3s)-3-(3-(2-(3-methylisoxazol-5-yl)acetamido)-1H-pyrazol-5-yl)cyclobutyl)methyl isopropylcarbamate (87 mg, 0.23 mmol, 70%) as a white amorphous solid.

m/z (ES⁺) [M+H]⁺=376.25; HPLC tR=1.168 min.

¹H NMR (400 MHz, DMSO-d₆) 12.14 (s, 1H), 10.64 (s, 1H), 7.05 (d, J=7.9 Hz, 1H), 6.35 (s, 1H), 6.23 (s, 1H), 4.04 (d, J=7.2 Hz, 2H), 3.83 (s, 2H), 3.57 (ddt, J=21.7, 15.8, 7.4 Hz, 3H), 2.46 (s, 1H), 2.21 (s, 4H), 2.16 (d, J=8.1 Hz, 3H), 1.05 (d, J=6.5 Hz, 6H).

Lyophilization yielded ((1s,3s)-3-(3-(2-(3-methylisoxazol-5-yl)acetamido)-1H-pyrazol-5-yl)cyclobutyl)methyl isopropylcarbamate (33.7 mg, 89.8 μmol, 27.0%) as a white amorphous solid.

m/z (ES⁺) [M+H]⁺=376.20; HPLC tR=1.128 min.

¹H NMR (400 MHz, DMSO-d₆) 12.14 (s, 1H), 10.64 (s, 1H), 7.05 (d, J=7.9 Hz, 1H), 6.35 (s, 1H), 6.23 (s, 1H), 4.04 (d, J=7.2 Hz, 2H), 3.83 (s, 2H), 3.57 (ddt, J=21.7, 15.8, 7.4 Hz, 3H), 2.58 (s, 1H), 2.21 (s, 4H), 2.16 (d, J=8.1 Hz, 3H), 1.05 (d, J=6.5 Hz, 6H).

### Example 22

### N-(5-((1R,3r,5S,6r)-6-(dimethylcarbamoyl)bicyclo[3.1.0]hexan-3-yl)-1H-pyrazol-3-yl)-3-(methoxymethyl)-1-methyl-1H-pyrazole-5-carboxamide

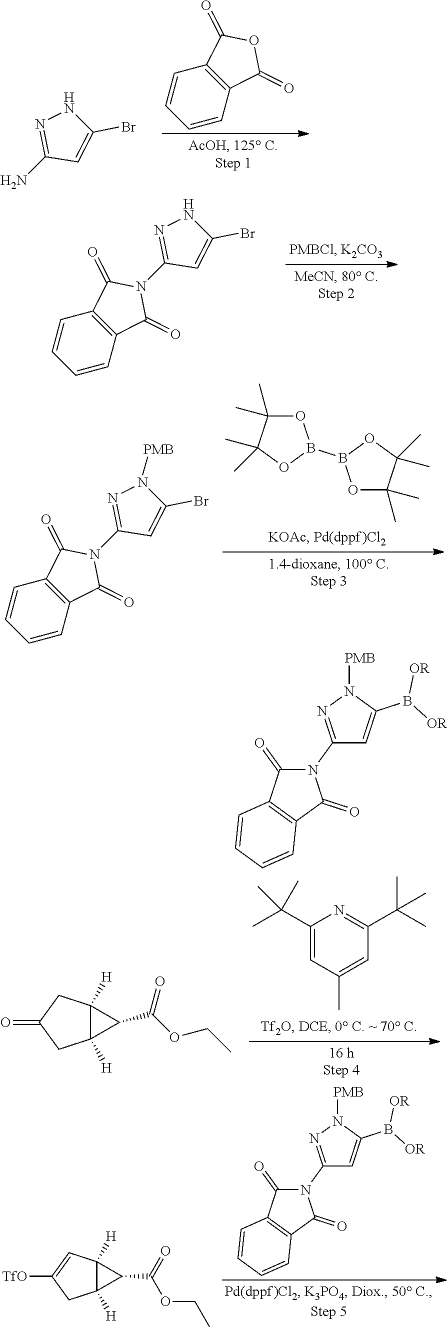

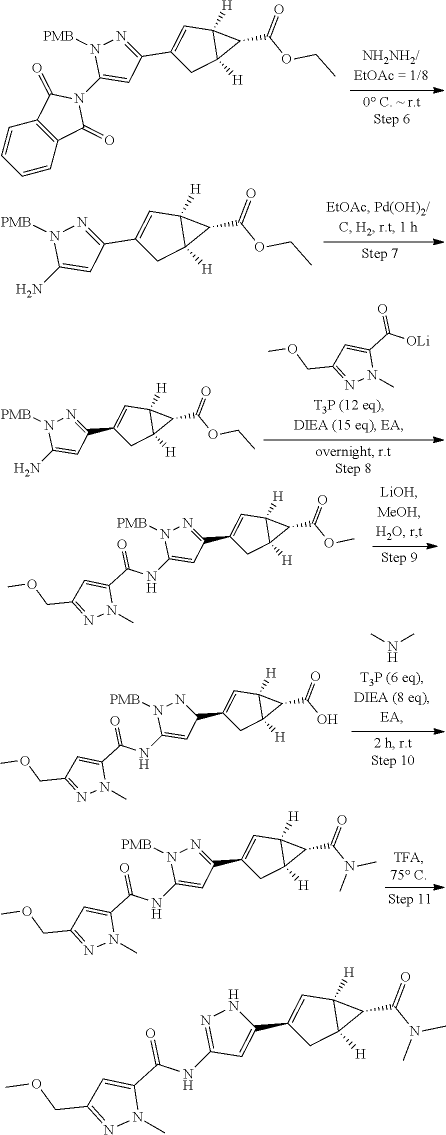

### 2-(5-bromo-1H-pyrazol-3-yl)isoindoline-1,3-dione

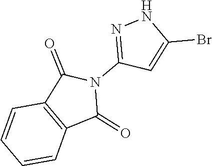 [SMILES: O=C1c2ccccc2C(=O)N1c1cc(Br)[nH]n1]

Step 1: 5-bromo-1H-pyrazol-3-amine (2.5 g, 15 mmol) and isobenzofuran-1,3-dione (2.7 g, 19 mmol) were combined in AcOH (25 mL) at room temperature under nitrogen atmosphere. The reaction mixture was stirred for 16 h at 125° C. The mixture was cooled to r.t., diluted with water, and extracted with ethyl acetate. The combined organic layer was washed with brine, dried over sodium sulfate, and concentrated under reduced pressure to afford 2-(5-bromo-1H-pyrazol-3-yl)isoindoline-1,3-dione (4.4 g, 15 mmol, 98%) as a yellow solid.

m/z (ES⁺) [M+H]⁺=303.20; HPLC tR=0.962 min.

### 2-(5-bromo-1-(4-methoxybenzyl)-1H-pyrazol-3-yl)isoindoline-1,3-dione

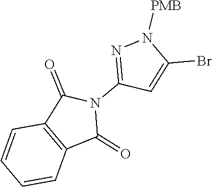

Step 2: To a solution of 2-(5-bromo-1H-pyrazol-3-yl)isoindoline-1,3-dione (4 g, 0.01 mol) and K₂CO₃ (6 g, 0.04 mol) in MeCN (30 mL) was added 1-(chloromethyl)-4-methoxybenzene (3 g, 0.02 mol) at room temperature under nitrogen atmosphere. The reaction mixture was stirred for 16 h at 80° C. The mixture was cooled to r.t. The resulting mixture was filtered, the filter cake was washed with EA (3×250 mL). The filtrate was concentrated under reduced pressure. The residue was purified by silica gel column chromatography, eluted with PE/EA (2/1) to afford 2-(5-bromo-1-(4-methoxybenzyl)-1H-pyrazol-3-yl)isoindoline-1,3-dione (5 g, 0.01 mol, 90%) as an off-white solid.

m/z (ES⁺) [M+H]⁺=413.95; HPLC tR=0.939 min.

### (3-(1,3-dioxoisoindolin-2-yl)-1-(4-methoxybenzyl)-1H-pyrazol-5-yl)boronic acid and 2-(1-(4-methoxybenzyl)-5-(4,4,5,5-tetramethyl-1,3,2-dioxaborolan-2-yl)-1H-pyrazol-3-yl)isoindoline-1,3-dione

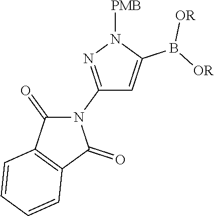

Step 3: To a solution of 2-(5-bromo-1-(4-methoxybenzyl)-1H-pyrazol-3-yl)isoindoline-1,3-dione (5 g, 0.01 mol) and 4,4,4′,4′,5,5,5′,5′-octamethyl-2,2′-bi(1,3,2-dioxaborolane) (6 g, 0.02 mol) in 1,4-dioxane (20 mL) was added potassium acetate (4 g, 0.04 mol) and PdCl₂(dppf)-CH₂C12 adduct (1 g, 1 mmol) at room temperature under nitrogen atmosphere. The reaction mixture was stirred for 16 h at 100° C. The mixture was cooled to r.t. The resulting mixture was filtered, and the filter cake was washed with EA (3×250 mL). The filtrate was concentrated under reduced pressure. The residue was purified by silica gel column chromatography, eluted with PE/EA (1/1) to afford a mixture of (3-(1,3-dioxoisoindolin-2-yl)-1-(4-methoxybenzyl)-1H-pyrazol-5-yl)boronic acid and 2-(1-(4-methoxybenzyl)-5-(4,4,5,5-tetramethyl-1,3,2-dioxaborolan-2-yl)-1H-pyrazol-3-yl)isoindoline-1,3-dione (2.6 g, 4.5 mmol, 40%, 80% purity) as an off-white solid.

m/z (ES⁺) [M+H]⁺=378.25; HPLC tR=0.951 min (boronic acid) ethyl (1S,5S,6R)-3-(((trifluoromethyl)sulfonyl)oxy)bicyclo[3.1.0]hex-2-ene-6-carboxylate

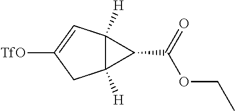 [SMILES: CCOC(=O)[C@H]1[C@@H]2C=C(OS(=O)(=O)C(F)(F)F)C[C@@H]21]

Step 4: To a solution of ethyl (1R,5S,6r)-3-oxobicyclo[3.1.0]hexane-6-carboxylate (2.5 g, 15 mmol) and 2,6-di-tert-butyl-4-methylpyridine (5.5 g, 27 mmol) in DCE (30 mL) at 0° C. was added trifluoromethanesulfonic anhydride (6.3 g, 22 mmol) drop wise. The reaction was allowed to stir at 70° C. for overnight. The resulting mixture was allowed to cool down to room temperature and diluted with DCM, washed with 10% citric acid, water, and brine. The organic phase was concentrated under reduced pressure. The crude was purified by reverse flash chromatography with the following conditions: column, C18 silica gel; mobile phase, ACN in water, 40% to 90% gradient in 10 min; detector, UV 254 nm to afford ethyl (1S,5S,6R)-3-(((trifluoromethyl)sulfonyl)oxy)bicyclo[3.1.0]hex-2-ene-6-carboxylate (840 mg, 2.80 mmol, 19%) as brown oil.

m/z (ES⁺) [M+H]⁺=301.10; HPLC tR=0.954 min.

### ethyl (1S,5S,6R)-3-(5-(1,3-dioxoisoindolin-2-yl)-1-(4-methoxybenzyl)-1H-pyrazol-3-yl)bicyclo[3.1.0]hex-2-ene-6-carboxylate

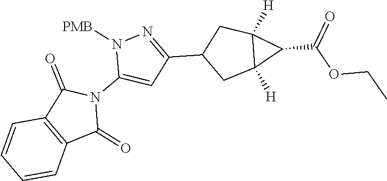

Step 5: To a vial was charged with ethyl (1S,5S,6R)-3-(((trifluoromethyl)sulfonyl)oxy)bicyclo[3.1.0]hex-2-ene-6-carboxylate (500 mg, 1.67 mmol), (3-(1,3-dioxoisoindolin-2-yl)-1-(4-methoxybenzyl)-1H-pyrazol-5-yl)boronic acid (816 mg, 2.16 mmol), K₃PO₄(1.06 g, 5.00 mmol) and [1,1′-bis(diphenylphosphino)ferrocene]dichloropalladium(II) complex with dichloromethane (136 mg, 167 μmol) in 1,4-dioxane (10 mL). The reaction was purged with N₂ three times before heating 50° C. for 16 hours. The crude was diluted with EtOAc, washed with 10% citric acid, water, and brine. The resulting mixture was concentrated under reduced pressure. The residue was purified by silica gel column chromatography, eluted with PE/EA (1:3) to afford ethyl (1S,5S,6R)-3-(5-(1,3-dioxoisoindolin-2-yl)-1-(4-methoxybenzyl)-1H-pyrazol-3-yl)bicyclo[3.1.0]hex-2-ene-6-carboxylate (900 mg, 1.5 mmol, 89%, 80% purity) as a white solid.

m/z (ES⁺) [M+H]⁺=484.30; HPLC tR=1.253 min.

### ethyl (1S,5S,6R)-3-(5-amino-1-(4-methoxybenzyl)-1Hpyrazol-3-yl)bicyclo[3.1.0]hex-2-ene-6-carboxylate

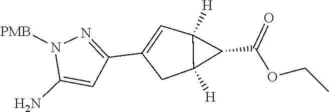

Step 6: A round bottomed flask was charged with EtOAc (8 mL) and hydrazinehydrate (1 mL) and a stirbar. Then ethyl (1S,5S,6R)-3-(5-(1,3-dioxoisoindolin-2-yl)-1-(4-methoxybenzyl)-1H-pyrazol-3-yl)bicyclo[3.1.0]hex-2-ene-6-carboxylate (850 mg, 1.76 mmol) was added at 0° C., and the resulting solution was stirred for 1 h at 0-25° C. The reaction mixture was diluted with water (20 mL), and the aqueous phase was extracted with EA (30 mL) three times. The combined organic layers were washed with brine, dried over sodium sulfate, filtered, and concentrated in vacuo. The resulting reaction mixture was purified by reverse flash chromatography with the following conditions: column, C18 silica gel; mobile phase, MeCN in water, 10% to 50% gradient in 10 min; detector, UV 254 nm to afford ethyl (1S,5S,6R)-3-(5-amino-1-(4-methoxybenzyl)-1Hpyrazol-3-yl)bicyclo[3.1.0]hex-2-ene-6-carboxylate (530 mg, 1.50 mmol, 85.3%) as a white solid.

m/z (ES⁺) [M+H]⁺=354.20; HPLC tR=0.939 min.

### ethyl (1R,3r,5S,6r)-3-(5-amino-1-(4-methoxybenzyl)-1H-pyrazol-3-yl)bicyclo[3.1.0]hexane-6-carboxylate

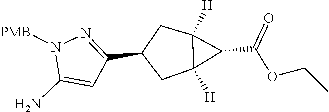

Step 7: To a solution of ethyl (1S,5S,6R)-3-(5-amino-1-(4-methoxybenzyl)-1H-pyrazol-3-yl)bicyclo[3.1.0]hex-2-ene-6-carboxylate (450 mg, 1.27 mmol) in EtOAc (10 mL) was added Pd(OH)₂/C (89.4 mg) under nitrogen atmosphere in a round bottomed flask. The mixture was hydrogenated at room temperature for 1 hour under hydrogen atmosphere using a hydrogen balloon, filtered through a Celite pad, and concentrated under reduced pressure to afford ethyl (1R,3r,5S,6r)-3-(5-amino-1-(4-methoxybenzyl)-1H-pyrazol-3-yl)bicyclo[3.1.0]hexane-6-carboxylate (430 mg, 1.21 mmol, 95.1%) as a yellow oil.

m/z (ES⁺) [M+H]⁺=356.25; HPLC tR=0.882 min.

### ethyl (1R,3r,5S,6r)-3-(1-(4-methoxybenzyl)-5-(3-(methoxymethyl)-1-methyl-1H-pyrazole-5-carboxamido)-1H-pyrazol-3-yl)bicyclo[3.1.0]hexane-6-carboxylate

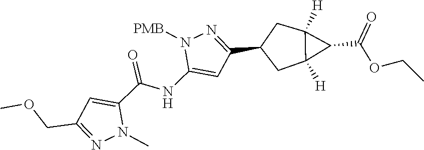

Step 8: To a mixture of ethyl (1R,3r,5S,6r)-3-(5-amino-1-(4-methoxybenzyl)-1H-pyrazol-3-yl)bicyclo[3.1.0]hexane-6-carboxylate (450 mg, 1.27 mmol), lithium 3-(methoxymethyl)-1-methyl-1H-pyrazole-5-carboxylate (268 mg, 1.52 mmol), and DIEA (2.45 g, 19.0 mmol) in EtOAc (10 mL) was added T3P (9.67 g, 50% Wt, 15.2 mmol) dropwised at 0° C. under nitrogen atmosphere. The mixture was stirred for 12 hours at 25° C. and concentrated under reduced pressure. The residue was purified by reverse phase flash with the following conditions: column, C18 silica gel; mobile phase, ACN in water, 10% to 80% gradient in 15 min; detector, UV 254 nm. to afford ethyl (1R,3r,5S,6r)-3-(1-(4-methoxybenzyl)-5-(3-(methoxymethyl)-1-methyl-1H-pyrazole-5-carboxamido)-1H-pyrazol-3-yl)bicyclo[3.1.0]hexane-6-carboxylate (430 mg, 847 μmol, 66.9%) as a light yellow oil.

m/z (ES⁺) [M+H]⁺=508.35; HPLC tR=1.129 min.

### (1R,3r,5S,6r)-3-(1-(4-methoxybenzyl)-5-(3-(methoxymethyl)-1-methyl-1H-pyrazole-5-carboxamido)-1H-pyrazol-3-yl)bicyclo[3.1.0]hexane-6-carboxylic acid

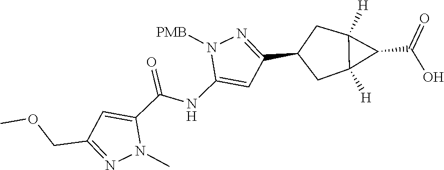

Step 9: A round bottomed flask was charged with ethyl (1R,3r,5S,6r)-3-(1-(4-methoxybenzyl)-5-(3-(methoxymethyl)-1-methyl-1H-pyrazole-5-carboxamido)-1H-pyrazol-3-yl)bicyclo[3.1.0]hexane-6-carboxylate (275 mg, 542 μmol) in MeOH (2 mL) and a stirbar. LiOH (104 mg, 4.33 mmol) in MeOH:H₂O (8 mL) was added, and the solution was stirred for 16 hours at 25° C. The resulting mixture was concentrated in vacuo. The residue was acidified to pH −6 with HCl (aq. 3 M). The resulting mixture was purified by reverse flash chromatography with the following conditions: column, C18 silica gel; mobile phase, ACN in water, 0% to 50% gradient in 15 min; detector, UV 254 nm. to afford (1R,3r,5S,6r)-3-(1-(4-methoxybenzyl)-5-(3-(methoxymethyl)-1-methyl-1H-pyrazole-5-carboxamido)-1H-pyrazol-3-yl)bicyclo[3.1.0]hexane-6-carboxylic acid (240 mg, 500 μmol, 92.4%) as a light yellow solid.

m/z (ES⁺) [M+H]⁺=480.35; HPLC tR=0.972 min.

### N-(3-((1R,3r,5S,6r)-6-(dimethylcarbamoyl)bicyclo[3.1.0]hexan-3-yl)-1-(4-methoxybenzyl)-1H-pyrazol-5-yl)-3-(methoxymethyl)-1-methyl-1H-pyrazole-5-carboxamide

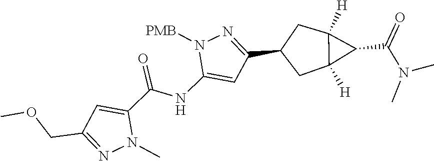

Step 10: To a mixture of (1R,3r,5S,6r)-3-(1-(4-methoxybenzyl)-5-(3-(methoxymethyl)-1-methyl-1H-pyrazole-5-carboxamido)-1H-pyrazol-3-yl)bicyclo[3.1.0]hexane-6-carboxylic acid (230 mg, 480 μmol), dimethylamine (1.20 mL, 2 M in THF, 2.40 mmol), and N-ethyl-Nisopropylpropan-2-amine (496 mg, 3.84 mmol) in EtOAc (10 mL) was added T3P (916 mg, 50% Wt, 2.88 mmol) drop wise at 0° C. under nitrogen atmosphere. The mixture was stirred for 2 hours at 25° C. and was concentrated under reduced pressure. The residue was purified by reverse phase flash with the following conditions: column, C18 silica gel; mobile phase, ACN in water, 10% to 80% gradient in 15 min; detector, UV 254 nm to afford N-(3-((1R,3r,5S,6r)-6-(dimethylcarbamoyl)bicyclo[3.1.0]hexan-3-yl)-1-(4-methoxybenzyl)-1H-pyrazol-5-yl)-3-(methoxymethyl)-1-methyl-1H-pyrazole-5-carboxamide (210 mg, 415 μmol, 86.4%) as a light yellow oil.

m/z (ES⁺) [M+H]⁺=507.40; HPLC tR=1.002 min.

### N-(5-((1R,3r,5S,6r)-6-(dimethylcarbamoyl)bicyclo[3.1.0]hexan-3-yl)-1H-pyrazol-3-yl)-3-(methoxymethyl)-1-methyl-1H-pyrazole-5-carboxamide

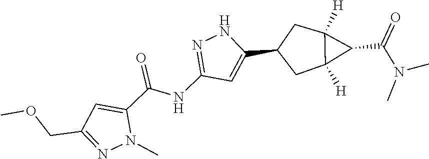

Step 11: A round bottomed flask was charged with N-(3-((1R,3r,5S,6r)-6-(dimethylcarbamoyl)bicyclo[3.1.0]hexan-3-yl)-1-(4-methoxybenzyl)-1Hpyrazol-5-yl)-3-(methoxymethyl)-1-methyl-1H-pyrazole-5-carboxamide (200 mg, 395 μmol) and a stirbar. TFA (6 mL) was added, and the solution was stirred for 5 hour at 75° C. The mixture was concentrated in vacuo. The residue was purified by reverse flash chromatography with the following conditions: column, C18 silica gel; mobile phase, MeCN in water, 10% to 100% gradient in 18 min; detector, UV 254 nm to afford N-(5-((1R,3r,5S,6r)-6-(dimethylcarbamoyl)bicyclo[3.1.0]hexan-3-yl)-1H-pyrazol-3-yl)-3-(methoxymethyl)-1-methyl-1H-pyrazole-5-carboxamide (135.1 mg, 349.6 μmol, 88.6%) as a white amorphous solid.

m/z (ES⁺) [M+H]⁺=387.20; HPLC tR=9.742 min.

¹H NMR (400 MHz, DMSO-d₆) δ 10.55 (s, 1H), 6.91 (s, 1H), 6.68 (s, 1H), 4.53 (s, 2H), 4.15 (s, 3H), 3.52 (s, 3H), 3.44 (td, J=9.5, 4.7 Hz, 1H), 3.02 (s, 3H), 2.91 (s, 3H), 2.46 (td, J=9.7, 9.2, 4.5 Hz, 2H), 2.07 (dd, J=13.9, 3.4 Hz, 2H), 2.00-1.93 (m, 2H), 1.65 (t, J=3.1 Hz, 1H).

### Example 23

### N-(5-((1S,3R)-3-((4-isopropylpyridazin-3-yl)oxy)cyclopentyl)-1H-pyrazol-3-yl)-2-(3-methylisoxazol-5-yl)acetamide and

### N-(5-((1R,3S)-3-((4-isopropylpyridazin-3-yl)oxy)cyclopentyl)-1H-pyrazol-3-yl)-2-(3-methylisoxazol-5-yl)acetamide

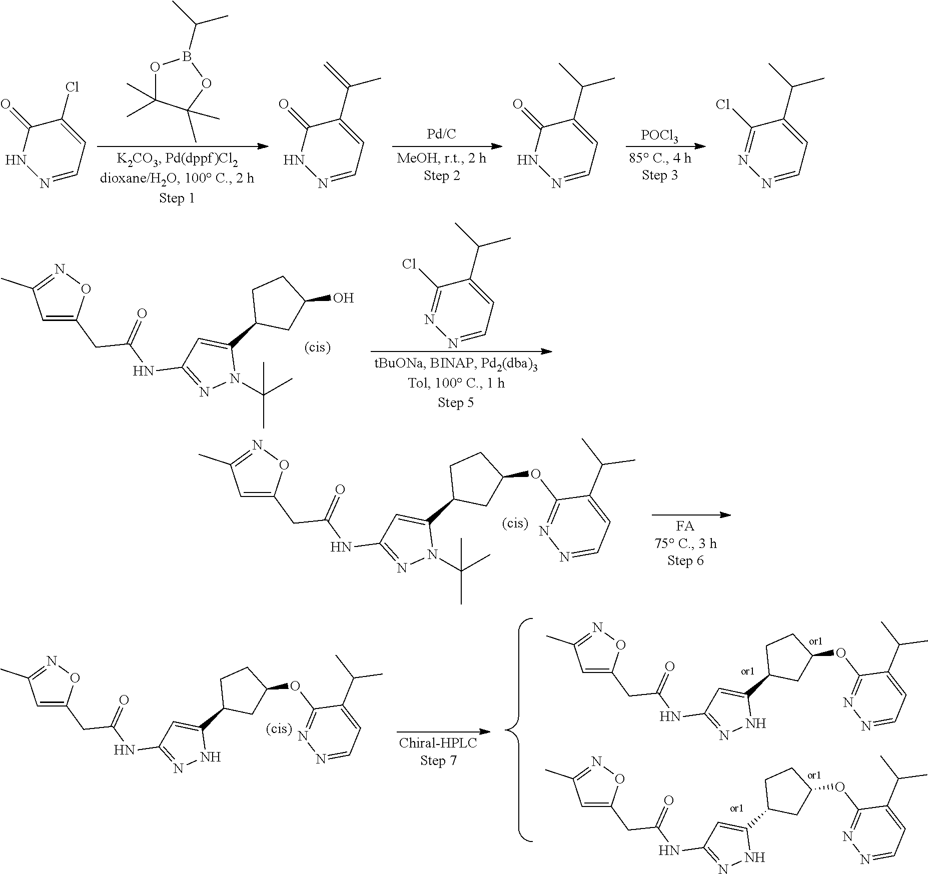

### 4-(prop-1-en-2-yl)pyridazin-3-ol

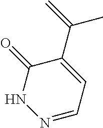

Step 1: A round-bottom flask was charged with 6-chloropyridazin-3(2H)-one (10 g, 1 Eq, 77 mmol), 4,4,5,5-tetramethyl-2-(prop-1-en-2-yl)-1,3,2-dioxaborolane (23 g, 1.8 Eq, 0.14 mol), potassium carbonate (32 g, 3 Eq, 0.23 mol), dioxane/H₂O (20 mL), and a stirbar before being evacuated and purged with nitrogen three times. Pd(dppf)C12 (2.8 g, 0.05 Eq, 3.8 mmol) was added. The mixture was stirred at 100° C. for 2 hours. The solution was concentrated. The resulting crude material was purified by Flash (acetonitrile/water). Lyophilization yielded 4-(prop-1-en-2-yl)pyridazin-3-ol (4.0 g, 31%) as a white amorphous solid.

m/z (ES⁺) [M+H]⁺=171.05 HPLC tR=0.725 min.

### 4-isopropylpyridazin-3(2H)-one

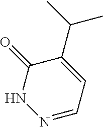

Step 2: A round-bottom flask was charged with 4-(prop-1-en-2-yl)pyridazin-3-ol (4 g, 1 Eq, 0.03 mol), Pd/C (0.5 g), MeOH (20 mL) and a stirbar before being evacuated and purged with hydrogen three times. The mixture was stirred at 25° C. for 2 hours. The mixture was filtered, and the filtrate was concentrated to afford 4-isopropylpyridazin-3(2H)-one (3.7 g, 91%) as a yellow oil.

m/z (ES⁺) [M+H]⁺=139.10; HPLC tR=0.675 min.

### 3-chloro-4-isopropylpyridazine

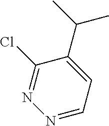

Step 3: A round bottomed flask was charged with 4-isopropylpyridazin-3-ol (3.7 g, 1 Eq, 27 mmol), POCl₃ (15 mL), and a stirbar, and the solution was stirred at 85° C. for 4 hours. The reaction mixture was poured into the ice water. The solution was extracted with EA three times. The organic phase was combined and concentrated. The resulting crude material was purified by Flash (acetonitrile/water). Lyophilization yielded 3-chloro-4-isopropylpyridazine (3.8 g, 24 mmol, 91%) as a black oil.

m/z (ES⁺) [M+H]⁺=157.05; HPLC tR=0.692 min.

### N-(1-(tert-butyl)-5-(cis-3-((4-isopropylpyridazin-3-yl)oxy)cyclopentyl)-1H-pyrazol-3-yl)-2-(3-methylisoxazol-5-yl)acetamide

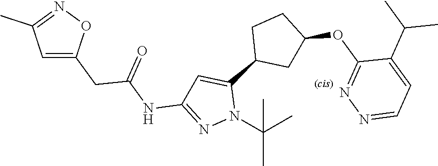 [SMILES: Cc1cc(CC(=O)Nc2cc([C@H]3CC[C@@H](Oc4nnccc4C(C)C)C3)n(C(C)(C)C)n2)on1]

Step 5: A resealable reaction vial was charged with 3-chloro-4-isopropylpyridazine (205 mg, 1.5 Eq, 1.31 mmol), N-(1-(tert-butyl)-5-(cis-3-hydroxycyclopentyl)-1H-pyrazol-3-yl)-2-(3-methylisoxazol-5-yl)acetamide (302 mg, 1 Eq, 873 μmol), Tol (5 mL), t-BuONa (0.25 g, 3 Eq, 2.62 mmol), BINAP (163 mg, 0.3 Eq, 262 μmol), Pd₂(dba)₃ (79.9 mg, 0.1 Eq, 87.3 μmol) and a stirbar before being evacuated and purged with nitrogen three times. The mixture was stirred at 100° C. for 1 hour. The solution was concentrated. The resulting crude material was purified by Flash (acetonitrile/water). Lyophilization yielded N-(1-(tert-butyl)-5-(cis-3-((4-isopropylpyridazin-3-yl)oxy)cyclopentyl)-1H-pyrazol-3-yl)-2-(3-methylisoxazol-5-yl)acetamide (183 mg, 392 μmol, 44.9%) as a yellow amorphous oil.

m/z (ES⁺) [M+H]⁺=467.30; HPLC tR=1.133 min.

### N-(5-(cis-3-((4-isopropylpyridazin-3-yl)oxy)cyclopentyl)-1H-pyrazol-3-yl)-2-(3-methylisoxazol-5-yl)acetamide

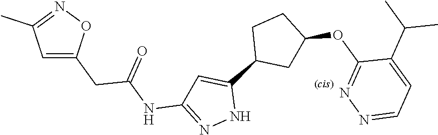 [SMILES: Cc1cc(CC(=O)Nc2cc([C@H]3CC[C@@H](Oc4nnccc4C(C)C)C3)[nH]n2)on1]

Step 6: A round bottomed flask was charged with N-(1-(tert-butyl)-5-(cis-3-((4-isopropylpyridazin-3-yl)oxy)cyclopentyl)-1H-pyrazol-3-yl)-2-(3-methylisoxazol-5-yl)acetamide (183 mg, 1 Eq, 392 μmol), FA (5 mL), and a stirbar, and the solution was stirred at 75° C. for 3 hours. The solution was concentrated. The resulting crude material was purified by prep-HPLC (Column: CHIRALPAK IE, 2X25 cm, 5 m; Mobile Phase A: Hex(0.5% 2M NH₃-MeOH)—HPLC, Mobile Phase B: EtOH: DCM=1:1—HPLC; Flow rate: 20 mL/min; Gradient: 60% B to 60% B in 11 min; Wave Length: 220/254 nm; RT1(min): 7.46; RT2(min): 9.00; Sample Solvent: EtOH: DCM=1:1—HPLC; Injection Volume: 0.55 mL; Number Of Runs: 4). Concentration in vacuo yielded N-(5-(cis-3-((4-isopropylpyridazin-3-yl)oxy)cyclopentyl)-1H-pyrazol-3-yl)-2-(3-methylisoxazol-5-yl)acetamide (80 mg, 0.19 mmol, 50%) as a yellow amorphous oil.

m/z (ES⁺) [M+H]⁺=411.25; HPLC tR=1.195 min.

### N-(5-((1S,3R)-3-((4-isopropylpyridazin-3-yl)oxy)cyclopentyl)-1H-pyrazol-3-yl)-2-(3-methylisoxazol-5-yl)acetamide and N-(5-((1R,3S)-3-((4-isopropylpyridazin-3-yl)oxy)cyclopentyl)-1H-pyrazol-3-yl)-2-(3-methylisoxazol-5-yl)acetamide

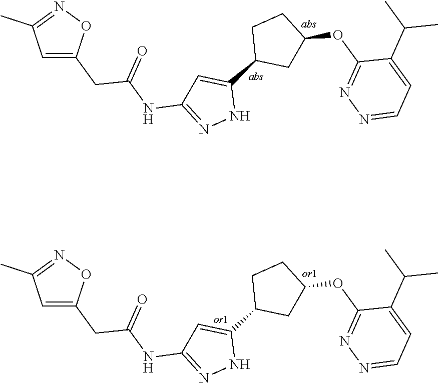

Step 7: N-(5-(cis-3-((4-isopropylpyridazin-3-yl)oxy)cyclopentyl)-1H-pyrazol-3-yl)-2-(3-methylisoxazol-5-yl)acetamide (80 mg, 1 Eq, 0.19 mmol) was purified by Chiral-HPLC(Column: CHIRALPAK IE, 2*25 cm, 5 m; Mobile Phase A: Hex(0.5% 2M NH3-MeOH)—HPLC, Mobile Phase B: EtOH: DCM=1:1—HPLC; Flow rate: 20 mL/min; Gradient: 60% B to 60% B in 11 min; Wave Length: 220/254 nm; RT1(min): 7.46; RT2(min): 9.00; Sample Solvent: EtOH: DCM=1:1—HPLC; Injection Volume: 0.55 mL; Number Of Runs: 4). Lyophilization yielded N-(5-((1S,3R)-3-((4-isopropylpyridazin-3-yl)oxy)cyclopentyl)-1H-pyrazol-3-yl)-2-(3-methylisoxazol-5-yl)acetamide (31.2 mg, 76.0 μmol, 39%) as a white amorphous solid.

m/z (ES⁺) [M+H]⁺=411.15; HPLC tR=0.655 min.

N-(5-((1R,3S)-3-((4-isopropylpyridazin-3-yl)oxy)cyclopentyl)-1H-pyrazol-3-yl)-2-(3-methylisoxazol-5-yl)acetamide (30.1 mg, 73.3 μmol, 38%) as a white amorphous solid.

m/z (ES⁺) [M+H]⁺=411.15; HPLC tR=0.663 min.

Additional compounds prepared according to the methods of Example 22 and 23 are depicted in Table 11 below.

TABLE 11
Additional Exemplary compounds

| | | | MS |
|---|---|---|---|
| | | | [M + |
| Compound | Structure | Proton NMR | 1] |
| 2-(3-methyl- isoxazol-5-yl)-N- (5-((1R,3S)- 3-(pyridin-3- yloxy) cyclopentyl)- | 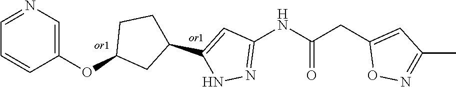 [SMILES: Cc1cc(CC(=O)Nc2cc([C@@H]3CC[C@H](Oc4cccnc4)C3)[nH]n2)on1] | ¹H NMR (400 MHz, Chloroform-d) 7.94 (d, J = 2.4 Hz, 2H), 7.42 (s, 1H), 6.83 (s, 1H), 6.60 (s, 1H), 5.24 (s, 1H), 4.68 (s, 1H), 3.83 (s, 1H), 3.25 (p, J = 8.2 Hz, 1H), 2.55 (dt, J = 15.1, 8.0 Hz, 1H), 2.18 (d, J = 8.0 Hz, 1H), 2.00- | 368.15 |
| 1H-pyrazol- | | 1.91 (m, 5H), 1.16 (t, J = 6.6 Hz, 6H). | |
| 3-yl)acetamide | | | |
| (1R,3S)-3-(3-(2-(3- methylisoxazol-5- yl)acetamido)- 1H-pyrazol-5- yl)cyclopentyl D-valinate | 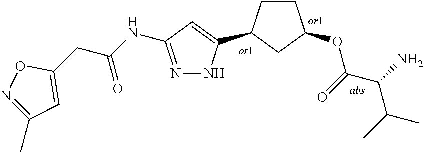 [SMILES: Cc1cc(CC(=O)Nc2cc([C@H]3CC[C@@H](OC(=O)[C@H](N)C(C)C)C3)[nH]n2)on1] | ¹H NMR (400 MHz, DMSO-d₆) 12.14 (s, 1H), 10.62 (s, 1H), 6.31 (s, 1H), 6.22 (s, 1H), 5.13 (dq, J = 9.6, 3.6 Hz, 1H), 3.83 (s, 2H), 3.09 (dd, J = 21.1, 6.9 Hz, 2H), 2.46 (dd, J = 8.0, 6.3 Hz, 1H), 2.20 (s, 3H), 2.09-1.86 (m, 2H), 1.86-1.77 (m, 2H), 1.81-1.67 (m, 2H), 1.62 (ddd, J = 13.4, 8.8, 4.5 Hz, 2H), 0.85 (d, J = 6.8 Hz, 3H), 0.78 (d, J = 6.8 Hz, 3H). | 390.30 |
| (1S,3R)-3-(3-(2-(3- methylisoxazol-5- yl)acetamido)- 1H-pyrazol-5- yl)cyclopentyl D-valinate | 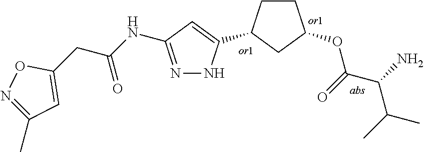 [SMILES: Cc1cc(CC(=O)Nc2cc([C@@H]3CC[C@H](OC(=O)[C@H](N)C(C)C)C3)[nH]n2)on1] | ¹H NMR (400 MHz, DMSO-d₆) 12.15 (s, 1H), 10.63 (s, 1H), 6.31 (s, 1H), 6.22 (s, 1H), 5.13 (tdd, J = 6.8, 4.3, 2.4 Hz, 1H), 3.83 (s, 2H), 3.17-3.06 (m, 1H), 3.06 (d, J = 5.3 Hz, 1H), 2.46 (td, J = 8.8, 7.4, 5.8 Hz, 1H), 2.20 (s, 3H), 2.10-1.76 (m, 3H), 1.79-1.61 (m, 5H), 0.86 (d, J = 6.8 Hz, 3H), 0.81 (d, J = 6.8 Hz, 3H). | 390.30 |
| (1R,3S)-3-(3-(2-(3- methylisoxazol-5- yl)acetamido)- 1H-pyrazol-5- yl)cyclopentyl L-valinate | 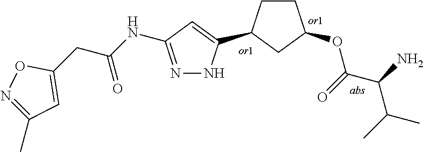 [SMILES: Cc1cc(CC(=O)Nc2cc([C@H]3CC[C@@H](OC(=O)[C@@H](N)C(C)C)C3)[nH]n2)on1] | ¹H NMR (400 MHz, DMSO-d₆) 12.14 (s, 1H), 10.62 (s, 1H), 6.31 (s, 1H), 6.22 (s, 1H), 5.13 (dq, J = 9.6, 3.6 Hz, 1H), 3.83 (s, 2H), 3.09 (dd, J = 21.1, 6.9 Hz, 2H), 2.46 (dd, J = 8.0, 6.3 Hz, 1H), 2.20 (s, 3H), 2.09-1.86 (m, 2H), 1.86-1.77 (m, 2H), 1.81-1.67 (m, 2H), 1.62 (ddd, J = 13.4, 8.8, 4.5 Hz, 2H), 0.85 (d, J = 6.8 Hz, 3H), 0.78 (d, J = 6.8 Hz, 3H). | 388.20 |
| (1S,3R)-3-(3-(2-(3- methylisoxazol-5- yl)acetamido)- 1H-pyrazol-5- yl)cyclopentyl L-valinate | 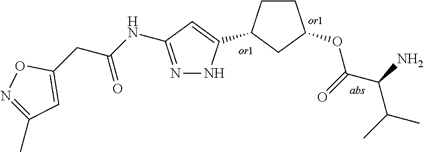 [SMILES: Cc1cc(CC(=O)Nc2cc([C@@H]3CC[C@H](OC(=O)[C@@H](N)C(C)C)C3)[nH]n2)on1] | ¹H NMR (400 MHz, DMSO-d₆) 12.15 (s, 1H), 10.63 (s, 1H), 6.31 (s, 1H), 6.22 (s, 1H), 5.13 (dtd, J = 6.8, 5.4, 4.3, 2.4 Hz, 1H), 3.83 (s, 2H), 3.15- 3.03 (m, 2H), 2.45 (dd, J = 8.3, 6.3 Hz, 1H), 2.20 (s, 3H), 2.10-1.61 (m, 7H), 0.86 (d, J = 6.8 Hz, 3H), 0.81 (d, J = 6.8 Hz, 3H). | 388.20 |
| (R)-2-hydroxy- 3-methyl-N- ((1R,3S)-3- (5-(2-(3- methylisoxazol-5- yl)acetamido)- 1H-pyrazol-3- yl)cyclopentyl) butanamide | 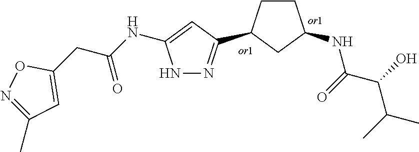 [SMILES: Cc1cc(CC(=O)Nc2cc([C@H]3CC[C@@H](NC(=O)[C@H](O)C(C)C)C3)n[nH]2)on1] | ¹H NMR (400 MHz, DMSO-d₆) 12.11 (s, 1H), 10.62 (s, 1H), 7.68 (d, J = 8.1 Hz, 1H), 6.31 (s, 1H), 6.22 (s, 1H), 5.24 (d, J = 5.6 Hz, 1H), 4.18 (q, J = 7.6 Hz, 1H), 3.83 (s, 2H), 3.63 (dd, J = 5.7, 3.9 Hz, 1H), 3.05 (d, J = 8.9 Hz, 1H), 2.28 (dd, J = 13.0, 6.7 Hz, 1H), 2.20 (s, 3H), 1.98-1.86 (m, 3H), 1.72- 1.50 (m, 3H), 0.89 (d, J = 6.9 Hz, 3H), 0.75 (d, J = 6.8 Hz, 3H). | 390.15 |
| (R)-2-hydroxy- 3-methyl-N- ((1R,3S)-3- (5-(2-(3- methylisoxazol-5- yl)acetamido)- 1H-pyrazol-3- yl)cyclopentyl) butanamide | 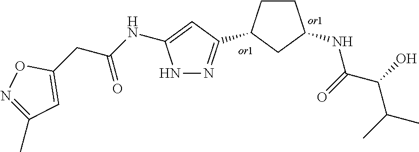 [SMILES: Cc1cc(CC(=O)Nc2cc([C@@H]3CC[C@H](NC(=O)[C@H](O)C(C)C)C3)n[nH]2)on1] | ¹H NMR (400 MHz, DMSO-d₆) 12.11 (s, 1H), 10.62 (s, 1H), 7.69 (d, J = 8.0 Hz, 1H), 6.32 (s, 1H), 6.22 (s, 1H), 5.25 (d, J = 5.7 Hz, 1H), 4.20-4.10 (m, 1H), 3.83 (s, 2H), 3.63 (dd, J = 5.7, 3.9 Hz, 1H), 3.02 (q, J = 8.5, 8.0 Hz, 1H), 2.27 (dt, J = 13.7, 7.2 Hz, 1H), 2.20 (s, 3H), 1.98-1.87 (m, 3H), 1.74-1.65 (m, 1H), 1.60-1.53 (m, 2H), 0.89 (d, J = 6.9 Hz, 3H), 0.76 (d, J = 6.8 Hz, 3H). | 390.25 |
| (S)-2-hydroxy- 3-methyl-N-rel- ((1R,3S)-3- (5-(2-(3- methylisoxazol-5- yl)acetamido)- 1H-pyrazol-3- yl)cyclopentyl) butanamide | 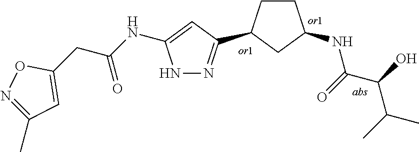 [SMILES: Cc1cc(CC(=O)Nc2cc([C@H]3CC[C@@H](NC(=O)[C@@H](O)C(C)C)C3)n[nH]2)on1] | ¹H NMR (400 MHz, DMSO-d₆) 12.11 (s, 1H), 10.63 (s, 1H), 7.69 (d, J = 8.0 Hz, 1H), 6.31 (s, 1H), 6.22 (s, 1H), 5.25 (d, J = 5.6 Hz, 1H), 4.18 (q, J = 8.4 Hz, 1H), 3.83 (s, 2H), 3.63 (dd, J = 5.7, 3.9 Hz, 1H), 3.02 (d, J = 7.9 Hz, 1H), 2.32-2.24 (m, 1H), 2.20 (s, 3H), 2.00-1.87 (m, 3H), 1.72-1.48 (m, 3H), 0.89 (d, J = 6.9 Hz, 3H), 0.75 (d, J = 6.8 Hz, 3H). | 390.35 |
| (S)-2-hydroxy- 3-methyl-N-rel- ((1R,3S)-3- (5-(2-(3- methylisoxazol-5- yl)acetamido)- 1H-pyrazol-3- yl)cyclopentyl) butanamide | 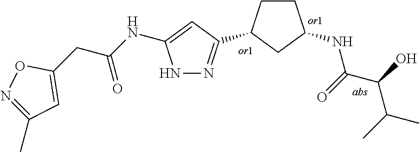 [SMILES: Cc1cc(CC(=O)Nc2cc([C@@H]3CC[C@H](NC(=O)[C@@H](O)C(C)C)C3)n[nH]2)on1] | ¹H NMR (400 MHz, DMSO-d₆) 12.12 (s, 1H), 10.62 (s, 1H), 7.70 (d, J = 8.0 Hz, 1H), 6.32 (s, 1H), 6.22 (s, 1H), 5.25 (d, J = 5.6 Hz, 1H), 4.18 (q, J = 7.6 Hz, 1H), 3.83 (s, 2H), 3.63 (dd, J = 5.7, 3.9 Hz, 1H), 3.02 (q, J = 8.8 Hz, 1H), 2.33-2.24 (m, 1H), 2.21 (s, 3H), 2.03-1.86 (m, 3H), 1.69 (d, J = 12.7 Hz, 1H), 1.62-1.50 (m, 2H), 0.89 (d, J = 6.9 Hz, 3H), 0.76 (d, J = 6.8 Hz, | 390.30 |
| | | 3H). | |

### Example 24

### rel-N-(5-((1R,3S)-3-((5-isopropyl-1H-pyrazol-4-yl)oxy)cyclopentyl)-1H-pyrazol-3-yl)-2-(3-methylisoxazol-5-yl)acetamide

### rel-N-(5-((1R,3S)-3-((5-isopropyl-1H-pyrazol-4-yl)oxy)cyclopentyl)-1H-pyrazol-3-yl)-2-(3-methylisoxazol-5-yl)acetamide

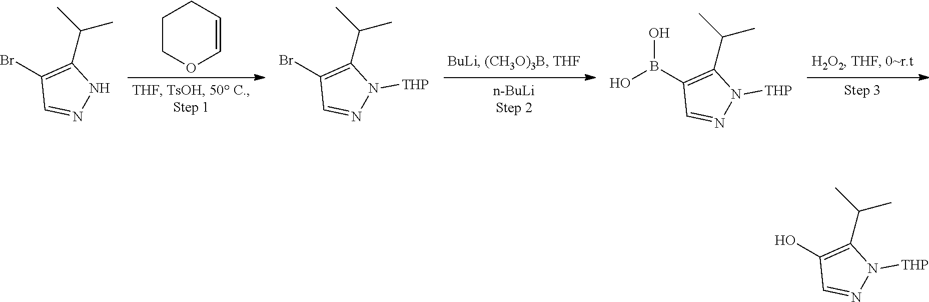

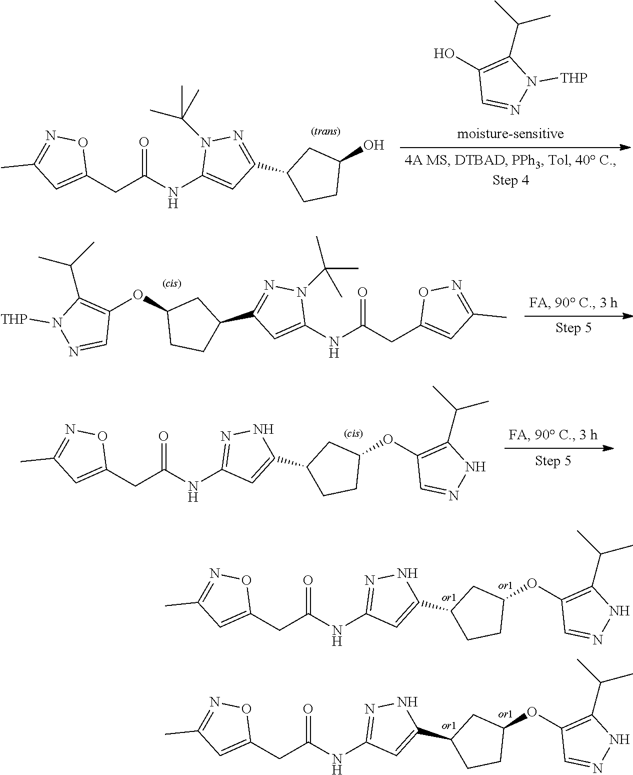

### 4-bromo-5-isopropyl-1-(tetrahydro-2H-pyran-2-yl)-1H-pyrazole

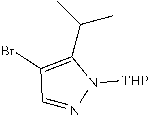

Step 1: A round bottomed flask was charged with 4-bromo-5-isopropyl-1H-pyrazole (3 g, 1 Eq, 0.02 mol), 4-methylbenzenesulfonic acid (0.5 g, 0.2 Eq, 3 mmol), 3,4-dihydro-2H-pyran (2 g, 1.5 Eq, 0.02 mol), THF (30 mL), and a stirbar. The solution was stirred at 50° C. for 2 hours. The reaction mixture was concentrated in vacuo, and the resulting crude material was purified by reverse flash chromatography with the following conditions: column, C18 silica gel; mobile phase, MeCN in water, 0% to 100% gradient in 25 min; detector, UV 220 nm.

Concentration in vacuo yielded 4-bromo-5-isopropyl-1-(tetrahydro-2H-pyran-2-yl)-1H-pyrazole (2.9 g, 11 mmol, 70%) as a white amorphous solid.

m/z (ES⁺) [M+H]⁺=272.95; HPLC tR=0.722 min.

### (5-isopropyl-1-(tetrahydro-2H-pyran-2-yl)-1H-pyrazol-4-yl)boronic acid

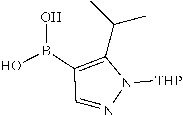

Step 2: To a mixture of 4-bromo-5-isopropyl-1-(tetrahydro-2H-pyran-2-yl)-1H-pyrazole (2.1 g, 7.7 mmol) in THE (20 mL) was added n-BuLi (3 mL of a solution 2.5 M in THF, 8 mmol) dropwise at −78° C. under nitrogen atmosphere. The mixture was stirred for 0.5 hour at −78° C. To the above mixture was added trimethyl borate (1.0 g, 9.6 mmol) dropwise at −78° C. The resulting mixture was stirred for additional 2 hours at −78° C. The reaction was quenched with NH₄Cl (sat. aq, 100 mL), and extracted with EA (3*100 mL). The combined organic layers were washed with brine (2*100 mL), dried over Na₂SO₄, filtered, and concentrated in vacuo. The residue was purified by reverse flash chromatography with the following conditions: column, C18 silica gel; mobile phase, MeCN in water, 0% to 100% gradient in 25 min; detector, UV 220 nm. Lyophilization for three days afforded (5-isopropyl-1-(tetrahydro-2H-pyran-2-yl)-1H-pyrazol-4-yl)boronic acid (620 mg, 2.60 mmol, 34%) as a white solid.

m/z (ES⁺) [M+H]⁺=239.10; HPLC tR=0.649 min.

### 5-isopropyl-1-(tetrahydro-2H-pyran-2-yl)-1H-pyrazol-4-ol

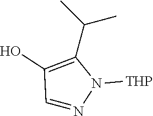

Step 3: To a stirred solution of (5-isopropyl-1-(tetrahydro-2H-pyran-2-yl)-1H-pyrazol-4-yl)boronic acid (620 mg, 2.60 mmol) in THE (8 mL) was added H₂O₂ (4 mL, 30% in H₂O) dropwise at 0° C. The reaction was allowed to warm to room temperature and stirred for 1 hour. The reaction was diluted with H₂O (20 mL), acidified with HCl (2 N), and extracted four times with DCM (50 mL). The combined organic layers were dried over Na₂SO₄ filtered and concentrated in vacuo. The residue was purified by reverse flash chromatography with the following conditions: column, C18 silica gel; mobile phase, ACN in water, 10% to 50% gradient in 10 min; detector, UV 254 nm. to afford 5-isopropyl-1-(tetrahydro-2H-pyran-2-yl)-1H-pyrazol-4-ol (500 mg, 2.38 mmol, 91.3%).

m/z (ES⁺) [M+H]⁺=211.00; HPLC tR=0.637 min.

### N-(1-(tert-butyl)-3-(cis-3-((5-isopropyl-1-(tetrahydro-2H-pyran-2-yl)-1H-pyrazol-4-yl)oxy)cyclopentyl)-1H-pyrazol-5-yl)-2-(3-methylisoxazol-5-yl)acetamide

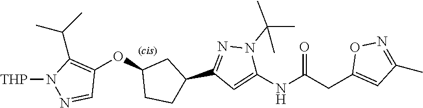

Step 4: To a stirred solution of N-(1-(tert-butyl)-3-(trans-3-hydroxycyclopentyl)-1H-pyrazol-5-yl)-2-(3-methylisoxazol-5-yl)acetamide (200 mg, 1 Eq, 577 μmol), 5-isopropyl-1-(tetrahydro-2H-pyran-2-yl)-1H-pyrazol-4-ol (121 mg, 1 Eq, 577 μmol), and triphenylphosphane (227 mg, 1.5 Eq, 866 μmol) in toluene (4 mL) with 4 A MS was added DTBAD (199 mg, 1.5 Eq, 866 μmol) dropwise at 0° C. under N₂ atmosphere. The resulting mixture was stirred for 4 hours at 40° C. under N₂ atmosphere. The resulting mixture was filtered, and the filter cake was washed with MeCN (5×3 mL). The filtrate was concentrated under reduced pressure. The residue was purified by reverse flash chromatography with the following conditions: column, C18 silica gel; mobile phase, ACN in water, 10% to 50% gradient in 10 min; detector, UV 254 nm. to give N-(1-(tert-butyl)-3-(cis-3-((5-isopropyl-1-(tetrahydro-2H-pyran-2-yl)-1H-pyrazol-4-yl)oxy)cyclopentyl)-1H-pyrazol-5-yl)-2-(3-methylisoxazol-5-yl)acetamide (102 mg, 189 μmol, 32.8%) as white solid.

m/z (ES⁺) [M+H]⁺=539.50; HPLC tR=1.096 min.

### N-(5-((1S,3R)-3-((5-isopropyl-1H-pyrazol-4-yl)oxy)cyclopentyl)-1Hpyrazol-3-yl)-2-(3-methylisoxazol-5-yl)acetamide

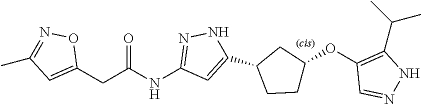 [SMILES: Cc1cc(CC(=O)Nc2cc([C@H]3CC[C@@H](Oc4cn[nH]c4C(C)C)C3)[nH]n2)on1]

Step 5: A solution of N-(1-(tert-butyl)-3-(cis-3-((5-isopropyl-1-(tetrahydro-2H-pyran-2-yl)-1H-pyrazol-4-yl)oxy)cyclopentyl)-1H-pyrazol-5-yl)-2-(3-methylisoxazol-5-yl)acetamide (165 mg, 306 μmol) in FA (5 mL) was stirred at 90° C. for 3 hours. The resulting mixture was concentrated to dryness and purified by reverse phase flash to afford N-(5-((1S,3R)-3-((5-isopropyl-1H-pyrazol-4-yl)oxy)cyclopentyl)-1Hpyrazol-3-yl)-2-(3-methylisoxazol-5-yl)acetamide (55 mg, 0.14 mmol, 45%) as a white solid.

m/z (ES⁺) [M+H]⁺=399.25; HPLC tR=0.905 min.

### rel-N-(5-((1R,3S)-3-((5-isopropyl-1H-pyrazol-4-yl)oxy)cyclopentyl)-1H-pyrazol-3-yl)-2-(3-methylisoxazol-5-yl)acetamide

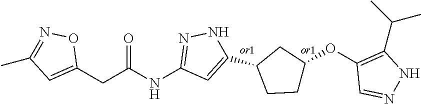 [SMILES: Cc1cc(CC(=O)Nc2cc([C@H]3CC[C@@H](Oc4cn[nH]c4C(C)C)C3)[nH]n2)on1]

N-(5-(cis-3-((5-isopropyl-1H-pyrazol-4-yl)oxy)cyclopentyl)-1H-pyrazol-3-yl)-2-(3-methylisoxazol-5-yl)acetamide (55 mg, 0.14 mmol) was purified by Prep-CHIRAL-HPLC following the condition: Column: CHIRALPAK IG, 2*25 cm, 5 m; Mobile Phase A: Hex(0.2% DEA)—HPLC, Mobile Phase B: MeOH: DCM=1:1; Flow rate: 20 mL/min; Gradient: 70% B to 70% B in 12 min; Wave Length: 220/254 nm; RT1(min): 6.23; Sample Solvent: MeOH: DCM=1:1; Injection Volume: 0.65 mL; Number Of Runs: 3. This resulted in rel-N-(5-((1R,3S)-3-((5-isopropyl-1H-pyrazol-4-yl)oxy)cyclopentyl)-1H-pyrazol-3-yl)-2-(3-methylisoxazol-5-yl)acetamide (20 mg, 50 μmol, 73%) as white amorphous solid.

m/z (ES⁺) [M+H]⁺=399.25; HPLC tR=1.117 min.

¹H NMR (400 MHz, DMSO-d₆) δ 12.25-11.74 (m, 2H), 10.61 (s, 1H), 7.27 (s, 1H), 6.42-6.28 (m, 1H), 6.22 (s, 1H), 4.50 (s, 1H), 3.82 (s, 2H), 3.07 (p, J=8.7 Hz, 1H), 2.91 (d, J=31.8 Hz, 1H), 2.53 (s, 1H), 2.20 (s, 3H), 2.02 (d, J=7.6 Hz, 1H), 1.87 (d, J=8.8 Hz, 2H), 1.82-1.68 (m, 2H), 1.15 (dd, J=7.0, 2.7 Hz, 6H).

### rel-N-(5-((1R,3S)-3-((5-isopropyl-1H-pyrazol-4-yl)oxy)cyclopentyl)-1H-pyrazol-3-yl)-2-(3-methylisoxazol-5-yl)acetamide

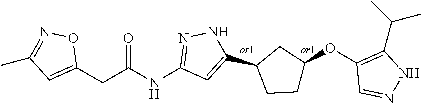 [SMILES: Cc1cc(CC(=O)Nc2cc([C@@H]3CC[C@H](Oc4cn[nH]c4C(C)C)C3)[nH]n2)on1]

N-(5-(cis-3-((5-isopropyl-1H-pyrazol-4-yl)oxy)cyclopentyl)-1H-pyrazol-3-yl)-2-(3-methylisoxazol-5-yl)acetamide (55 mg, 0.14 mmol) was purified by Prep-CHIRAL-HPLC following the condition: Column: CHIRALPAK IG, 2*25 cm, 5 m; Mobile Phase A: Hex(0.2% DEA)—HPLC, Mobile Phase B: MeOH: DCM=1:1; Flow rate: 20 mL/min; Gradient: 70% B to 70% B in 12 min; Wave Length: 220/254 nm; RT2(min): 9.18; Sample Solvent: MeOH: DCM=1:1; Injection Volume: 0.65 mL; Number Of Runs: 3. This afforded rel-N-(5-((1R,3S)-3-((5-isopropyl-1H-pyrazol-4-yl)oxy)cyclopentyl)-1H-pyrazol-3-yl)-2-(3-methylisoxazol-5-yl)acetamide (20 mg, 50 μmol, 73%) as white amorphous solid.

m/z (ES⁺) [M+H]⁺=399.25; HPLC tR=1.121 min.

¹H NMR (400 MHz, DMSO-d₆) δ 12.24-11.78 (m, 2H), 10.61 (s, 1H), 7.24 (d, J=39.1 Hz, 1H), 6.37-6.13 (m, 2H), 4.50 (s, 1H), 3.82 (s, 2H), 3.07 (p, J=8.8 Hz, 1H), 2.87 (s, 1H), 2.53 (d, J=2.0 Hz, 1H), 2.20 (s, 3H), 2.02 (d, J=7.3 Hz, 1H), 1.87 (d, J=9.4 Hz, 2H), 1.81-1.66 (m, 2H), 1.15 (dd, J=7.0, 2.7 Hz, 6H).

### Example 25

### rel-(1R,3S)-3-(3-((2-(methoxymethyl)pyrazolo[1,5-a]pyrazin-4-yl)amino)-1H-pyrazol-5-yl)cyclopentyl isopropylcarbamate; and

### rel-(1R,3S)-3-(3-((2-(methoxymethyl)pyrazolo[1,5-a]pyrazin-4-yl)amino)-1H-pyrazol-5-yl)cyclopentyl isopropylcarbamate

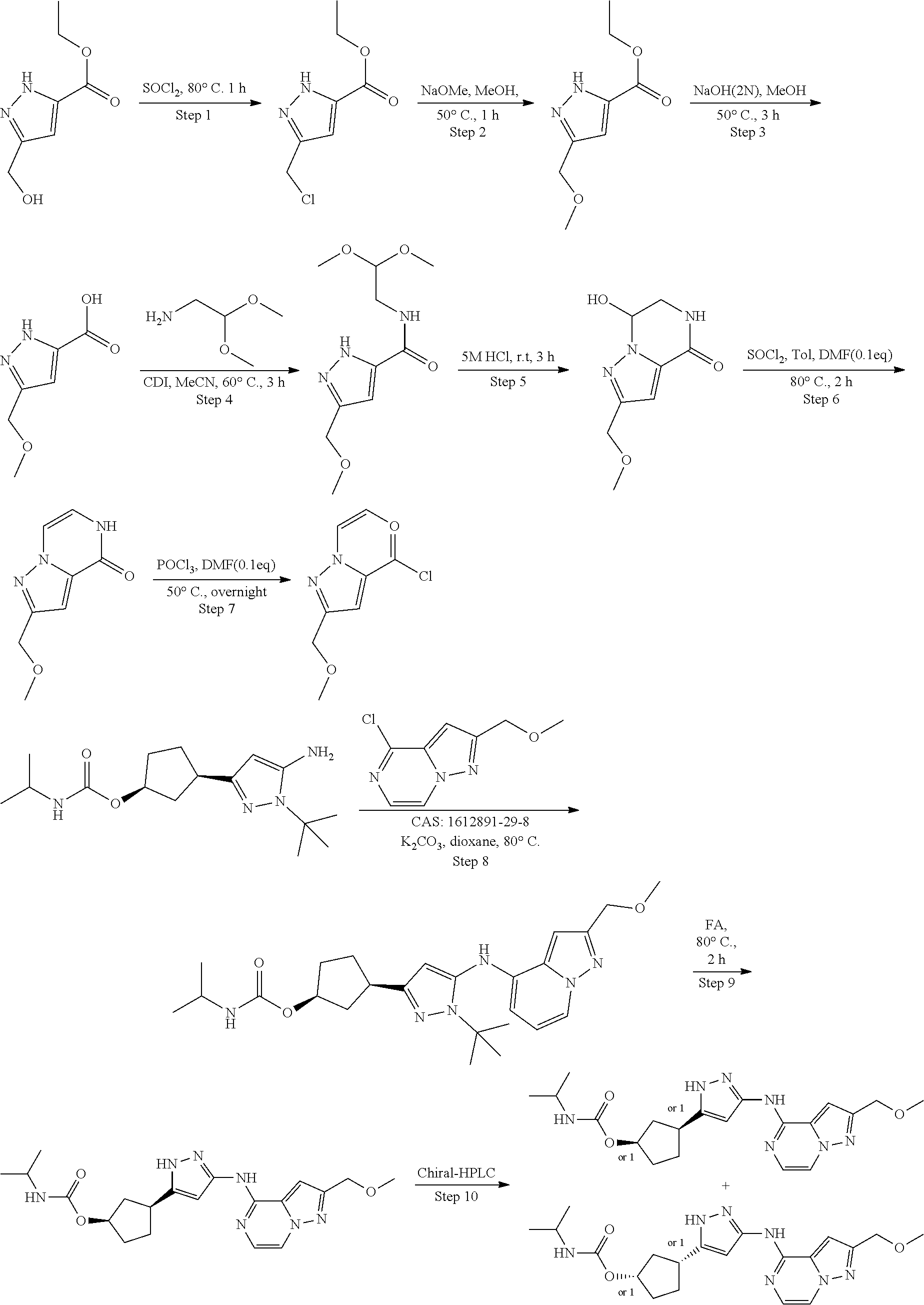

### ethyl 3-(chloromethyl)-1H-pyrazole-5-carboxylate

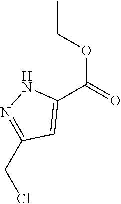

A resealable reaction vial was charged with ethyl 3-(hydroxymethyl)-1H-pyrazole-5-carboxylate (20 g, 1 Eq, 0.12 mol), SOCl₂, (150 mL), and a stirbar before being evacuated and purged with nitrogen three times. The mixture was stirred for 1 h at 80° C. The reaction mixture was poured into crushed ice, and the aqueous phase was extracted with EA (100 mL) three times. The combined organic layers were washed with brine, dried over sodium sulfate, filtered, and concentrated in vacuo. The crude product was purified by silica gel chromatography (10 g column; eluting with PE/EA; ratio:8/1). Concentration in vacuo resulted in ethyl 3-(chloromethyl)-1H-pyrazole-5-carboxylate (20 g, 0.11 mol, 90%) as a colourless oil.

m/z (ES⁺) [M+H]⁺=189.10; HPLC tR=0.869 min.

### ethyl 3-(methoxymethyl)-1H-pyrazole-5-carboxylate

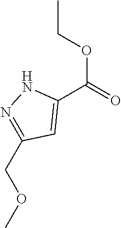

A resealable reaction vial was charged with ethyl 3-(hydroxymethyl)-1H-pyrazole-5-carboxylate (20 g, 1 Eq, 0.12 mol), SOCl₂, (150 mL) and a stirbar before being evacuated and purged with nitrogen three times. The mixture was stirred for 1 h at 80° C. The reaction mixture was poured into crushed ice, and the aqueous phase was extracted with EA (100 mL) three times. The combined organic layers were washed with brine, dried over sodium sulfate, filtered, and concentrated in vacuo. The crude product was purified by silica gel chromatography (10 g column; eluting with PE/EA; ratio:8/1). Concentration in vacuo resulted in ethyl 3-(chloromethyl)-1H-pyrazole-5-carboxylate (20 g, 0.11 mol, 90%) as a colourless oil.

m/z (ES⁺) [M+H]⁺=189.10; HPLC tR=0.869 min.

### 3-(methoxymethyl)-1H-pyrazole-5-carboxylic acid

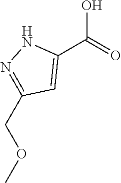

A resealable reaction vial was charged with ethyl 3-(methoxymethyl)-1H-pyrazole-5-carboxylate (12.8 g, 1 Eq, 69.5 mmol) in MeOH(30 mL), To the above solution NaOH (4.17 g, 52.1 L, 0.002 molar, 1.5 Eq, 104 mmol) in MeOH/H2O(2:1, 30 mL) was added, and the mixture was stirred for 2 hour at 50° C. The reaction mixture was concentrated in vacuo and extracted with EA (20 ml). The aqueous phase was acidified to pH 5 with HCl (1 M). The resulting mixture was extracted with EA (3×50 ml), dried over anhydrous Na₂SO₄. After filtration, the filtrate was concentrated under reduced pressure. The resulting mixture was concentrated under reduced pressure to afford 3-(methoxymethyl)-1H-pyrazole-5-carboxylic acid (4.3 g, 28 mmol, 40%) as white solid.

m/z (ES⁺) [M+H]⁺=157.10; HPLC tR=0.140 min.

### N-(2,2-dimethoxyethyl)-3-(methoxymethyl)-1H-pyrazole-5-carboxamide

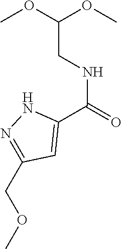

To a mixture of 3-(methoxymethyl)-1H-pyrazole-5-carboxylic acid (1.25 g, 1 Eq, 8.01 mmol) and in MeCN (10 mL) was added CDI (1.56 g, 1.2 Eq, 9.61 mmol) in portions at 25° C. under nitrogen atmosphere. The mixture was stirred for 1-2 h at 60° C. and 2,2-dimethoxyethan-1-amine (842 mg, 1 Eq, 8.01 mmol) was added. The mixture was stirred for 2 h at 60° C. The reaction mixture was diluted with H₂O (50 mL), and the aqueous phase was extracted with EA (100 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water). Concentration in vacuo resulted in N-(2,2-dimethoxyethyl)-3-(methoxymethyl)-1H-pyrazole-5-carboxamide (1.4 g, 5.8 mmol, 72%) as a white solid.

m/z (ES⁺) [M+H]⁺=244.20; HPLC tR=0.644 min.

### 7-hydroxy-2-(methoxymethyl)-6,7-dihydropyrazolo[1,5-a]pyrazin-4(5H)-one

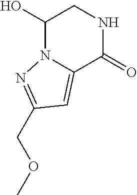

A resealable reaction vial was charged with N-(2,2-dimethoxyethyl)-3-(methoxymethyl)-1H-pyrazole-5-carboxamide (1.4 g, 1 Eq, 5.8 mmol), HCl (5 M) (25 mL) was added, and a stirbar before being evacuated and purged with nitrogen three times., and the mixture was stirred for 3 h at 25° C. The residue was purified by reverse flash chromatography with the following conditions: column, C18 silica gel; mobile phase, MeCN in water, 10% Concentration in vacuo resulted in 7-hydroxy-2-(methoxymethyl)-6,7-dihydropyrazolo[1,5-a]pyrazin-4(5H)-one (1 g, 5 mmol, 90%) as a white solid.

m/z (ES⁺) [M+H]⁺=198.15; HPLC tR=0.259 min.

### 2-(methoxymethyl)pyrazolo[1,5-a]pyrazin-4(5H)-one

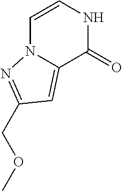

A round bottomed flask was charged with 7-hydroxy-2-(methoxymethyl)-6,7-dihydropyrazolo[1,5-a]pyrazin-4(5H)-one (1 g, 1 Eq, 5 mmol), toluene (20 mL), and a stirbar, and 4A-Ms (0.2 g, 1 Eq, 5 mmol), DMF (0.04 g, 0.04 mL, 0.1 Eq, 0.5 mmol), SOCl₂ (1 g, 0.7 mL, 2 Eq, 0.01 mol) were added at 0° C. The solution was stirred at 80° C. for 2 hour. The reaction mixture was filtered through a pad of Celite, the pad was washed with ACN, and the filtrate was concentrated in vacuo. The combined organic layers were washed with brine, dried over sodium sulfate, filtered, and concentrated to get 2-(methoxymethyl)pyrazolo[1,5-a]pyrazin-4(5H)-one (840 mg, 4.69 mmol, 90%) as a yellow amorphous solid.

m/z (ES⁺) [M+H]⁺=180.05; HPLC tR=0.399 min.

### 4-chloro-2-(methoxymethyl)pyrazolo[1,5-a]pyrazine

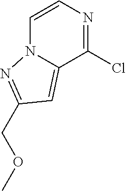

A round bottomed flask was charged with 2-(methoxymethyl)pyrazolo[1,5-a]pyrazin-4(5H)-one (840 mg, 1 Eq, 4.69 mmol), POCl₃ (15 mL) and a stirbar, and DMF (34.3 mg, 36.3 L, 0.1 Eq, 469 μmol) was added, and the solution was stirred at 50° C. for 16 hour. The reaction mixture was quenched with water. The resulting crude material was purified by Flash (acetonitrile/water). Concentration in vacuo afforded 4-chloro-2-(methoxymethyl)pyrazolo[1,5-a]pyrazine (600 mg, 3.04 mmol, 64.8%) as a blown amorphous solid.

m/z (ES⁺) [M+H]⁺=198.00; HPLC tR=0.696 min.

### (1S,3R)-3-(1-(tert-butyl)-5-((2-(methoxymethyl)pyrazolo[1,5-a]pyrazin-4-yl)amino)-1H-pyrazol-3-yl)cyclopentyl isopropylcarbamate

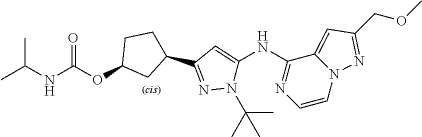 [SMILES: COCc1cc2c(Nc3cc([C@@H]4CC[C@H](OC(=O)NC(C)C)C4)nn3C(C)(C)C)nccn2n1]

4-chloro-2-ethoxypyrazolo[1,5-a]pyrazine (192 mg, 1 Eq, 973 μmol) and K₂CO₃ (403 mg, 3 Eq, 2.92 mmol) were added to a solution of (1S,3R)-3-(5-amino-1-(tert-butyl)-1H-pyrazol-3-yl)cyclopentyl isopropylcarbamate (300 mg, 1 Eq, 973 μmol) in 1,4-dioxane (10 mL). After bubbling nitrogen through the reaction mixture for 2 minutes, Pd-PEPPSI-IPentCl 2-methylpyridine (CAS No. 1612891-29-8, 81.4 mg, 0.1 Eq, 97.3 μmol) was added. The reaction mixture is heated at 80° C. for 16 hour with vigorous stirring. The residue was purified by reverse flash chromatography with the following conditions: column, C18 silica gel; mobile phase, MeCN in water, 10% to 100% gradient in 20 min; detector, UV 220 nm to afford (1S,3R)-3-(1-(tert-butyl)-5-((2-(methoxymethyl)pyrazolo[1,5-a]pyrazin-4-yl)amino)-1H-pyrazol-3-yl)cyclopentyl isopropylcarbamate (60 mg, 0.13 mmol, 13%) as a colourless oil.

m/z (ES⁺) [M+H]⁺=470.40; HPLC tR=0.628 min.

### (1R,3S)-3-(3-((2-(methoxymethyl)pyrazolo [1,5-a]pyrazin-4-yl)amino)-1H-pyrazol-5-yl)cyclopentyl isopropylcarbamate

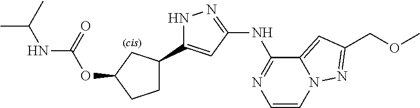 [SMILES: COCc1cc2c(Nc3cc([C@H]4CC[C@@H](OC(=O)NC(C)C)C4)[nH]n3)nccn2n1]

To a solution of (1S,3R)-3-(1-(tert-butyl)-5-((2-(methoxymethyl)pyrazolo[1,5-a]pyrazin-4-yl)amino)-1H-pyrazol-3-yl)cyclopentyl isopropylcarbamate (55 mg, 1 Eq, 0.12 mmol) was added FA (5 mL). The resulting solution was stirred at 80° C. for 2 h. LC/MS showed the reaction mixture was completed. The mixture was allowed to cool down to r.t. The resulting mixture was concentrated in vacuo. The residue was purified by reverse flash chromatography with the following conditions: column, C18 silica gel; mobile phase, MeCN in water, 10% to 50% gradient in 10 min; detector, UV 254 nm. to obtain (1R,3S)-3-(3-((2-(methoxymethyl)pyrazolo [1,5-a]pyrazin-4-yl)amino)-1H-pyrazol-5-yl)cyclopentyl isopropylcarbamate (30 mg, 72 μmol, 61%, 99% Purity) as an off-white solid.

m/z (ES⁺) [M+H]⁺=414.35; HPLC tR=0.848 min.

### rel-(1R,3S)-3-(3-((2-(methoxymethyl)pyrazolo[1,5-a]pyrazin-4-yl)amino)-1H-pyrazol-5-yl)cyclopentyl isopropylcarbamate

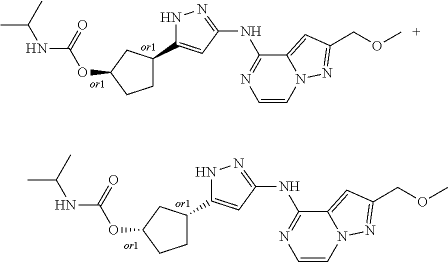

Cis-3-(3-((2-(methoxymethyl)pyrazolo[1,5-a]pyrazin-4-yl)amino)-1H-pyrazol-5-yl)cyclopentyl isopropyl carbamate (30 mg) was separated by chiral separation (HPLC) with the following conditions (Column: CHIRALPAK IG, 2*25 cm, 5 m; Mobile Phase A: Hex(0.2% DEA)—HPLC, Mobile Phase B: MeOH: DCM=1:1; Flow rate: 20 mL/min; Gradient: 30% B to 30% B in 20 min; Wave Length: 220/254 nm; RT1(min): 10.43; Sample Solvent: MeOH: DCM=1:1; Injection Volume: 2.4 mL; Number Of Runs: 1) to afford rel-(1R,3S)-3-(3-((2-(methoxymethyl)pyrazolo[1,5-a]pyrazin-4-yl)amino)-1H-pyrazol-5-yl)cyclopentyl isopropylcarbamate (8.2 mg, 20 μmol, 63%, 99.4% Purity) as a white solid and rel-(1R,3S)-3-(3-((2-(methoxymethyl)pyrazolo[1,5-a]pyrazin-4-yl)amino)-1H-pyrazol-5-yl)cyclopentyl isopropylcarbamate (7.2 mg, 17 μmol, 42%, 99.4% Purity) as a white solid.

First eluting isomer:

m/z (ES⁺) [M+H]⁺=414.35; HPLC tR=0.893 min.

¹H NMR (400 MHz) 12.10 (s, 1H), 9.97 (s, 1H), 8.02 (d, J=4.7 Hz, 1H), 7.38 (d, J=4.8 Hz, 1H), 7.28 (s, 1H), 6.97 (d, J=7.7 Hz, 1H), 6.62 (s, 1H), 5.02 (s, 1H), 4.56 (s, 2H), 3.58 (h, J=6.6 Hz, 1H), 3.33 (s, 4H), 3.09 (t, J=8.9 Hz, 1H), 2.10 (m, 1H), 1.91 (tt, J=9.0, 4.7 Hz, 1H), 1.75 (d, J=9.4 Hz, 2H), 1.04 (dd, J=6.7, 2.6 Hz, 6H).

Second eluting isomer:

m/z (ES⁺) [M+H]⁺=414.35; HPLC tR=0.899 min.

¹H NMR (400 MHz) 12.11 (s, 1H), 9.97 (s, 1H), 8.02 (d, J=4.7 Hz, 1H), 7.38 (d, J=4.8 Hz, 1H), 7.28 (s, 1H), 6.97 (d, J=7.8 Hz, 1H), 6.62 (s, 1H), 5.02 (s, 1H), 4.56 (s, 2H), 3.58 (h, J=6.7 Hz, 1H), 3.33 (s, 4H), 3.13 (m, 1H), 2.10 (m, 2H), 1.91 (tt, J=8.9, 4.7 Hz, 3H), 1.04 (dd, J=6.6, 2.7 Hz, 6H).

### Example 26

### 4-chloro-2-(methoxymethyl)-4,5-dihydropyrazolo[1,5-a]pyrazine

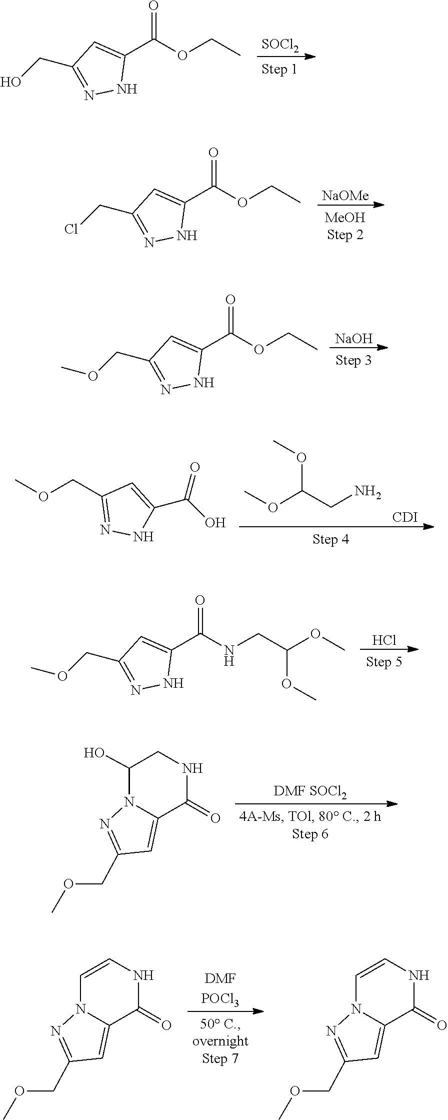

### ethyl 3-(chloromethyl)-1H-pyrazole-5-carboxylate

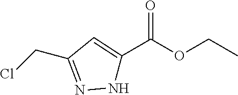

Step 1: A resealable reaction vial was charged with ethyl 3-(hydroxymethyl)-1H-pyrazole-5-carboxylate (20 g, 1 Eq, 0.12 mol), SOCl₂ (150 mL) and a stirbar were added before being evacuated and purged with nitrogen three times. The mixture was stirred for 1 h at 80° C. The reaction mixture was poured into crushed ice, and the aqueous phase was extracted with EA (100 mL) three times. The combined organic layers were washed with brine, dried over sodium sulfate, filtered, and concentrated in vacuo. The residue/crude product was purified by silica gel chromatography (10 g column; eluting with PE/EA; ratio:8/1). Concentration in vacuo resulted in ethyl 3-(chloromethyl)-1H-pyrazole-5-carboxylate (20 g, 0.11 mol, 90%) as a colourless oil.

### ethyl 3-(methoxymethyl)-1H-pyrazole-5-carboxylate

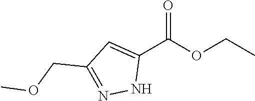

Step 2: To a mixture of ethyl 3-(chloromethyl)-1H-pyrazole-5-carboxylate (20 g, 1 Eq, 0.11 mol) in MeOH (200 mL) was added NaOMe (6.3 g, 1.1 Eq, 0.12 mol) in MeOH (15 mL) in portions at 0° C. under nitrogen atmosphere. The mixture was stirred for 10 min at 0° C. After, the mixture was stirred for 1 h at 50° C. The reaction mixture was concentrated in vacuo and diluted with H₂O (50 mL), and the aqueous phase was extracted with EA (50 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 10% to 50% gradient in 20 min; detector, UV 250 nm. Concentration in vacuo resulted in ethyl 3-(methoxymethyl)-1H-pyrazole-5-carboxylate (12.8 g, 41 mmol, 39%, 59% Purity) as a colourless oil.

### 3-(methoxymethyl)-1H-pyrazole-5-carboxylic acid

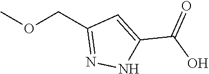

Step 3: A resealable reaction vial was charged with ethyl 3-(methoxymethyl)-1H-pyrazole-5-carboxylate (12.8 g, 1 Eq, 69.5 mmol) in MeOH(30 mL). To above solution, NaOH (4.17 g, 52.1 mL, 0.002 molar, 1.5 Eq, 104 mmol) in MeOH/H₂O (2:1, 30 mL) was added, and the mixture was stirred for 2 hour at 50° C. The reaction mixture was concentrated in vacuo and extracted with EA (20 ml). The aqueous phase was acidified to pH 5 with HCl (1 M). The resulting mixture was extracted with EA (3×50 ml]) and dried over anhydrous Na₂SO₄. After filtration, the filtrate was concentrated under reduced pressure. The resulting mixture was concentrated under reduced pressure to affod 3-(methoxymethyl)-1H-pyrazole-5-carboxylic acid (4.3 g, 28 mmol, 40%) as white solid.

### N-(2,2-dimethoxyethyl)-3-(methoxymethyl)-1H-pyrazole-5-carboxamide

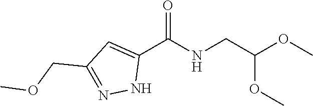

Step 4: To a mixture of 3-(methoxymethyl)-1H-pyrazole-5-carboxylic acid (1.25 g, 1 Eq, 8.01 mmol) in MeCN (10 mL) was added CDI (1.56 g, 1.2 Eq, 9.61 mmol) in portions at 25° C. under nitrogen atmosphere. The mixture was stirred for 1-2 h at 60° C., followed by the addition of 2,2-dimethoxyethan-1-amine (842 mg, 1 Eq, 8.01 mmol). The mixture was stirred for 2 h at 60° C. The reaction mixture was diluted with H₂O (50 mL), and the aqueous phase was extracted with EA (100 mL) three times. The combined organic layers were washed with brine, dried over sodium sulfate, filtered, and concentrated in vacuo. The residue was purified by reverse flash chromatography with the following conditions: column, C18 silica gel; mobile phase, MeCN in water, 10%. Concentration in vacuo resulted in N-(2,2-dimethoxyethyl)-3-(methoxymethyl)-1H-pyrazole-5-carboxamide (1.4 g, 5.8 mmol, 72%) as a white solid.

### 7-hydroxy-2-(methoxymethyl)-6,7-dihydropyrazolo[1,5-a]pyrazin-4(5H)-one

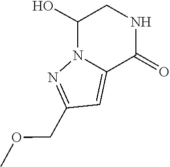

Step 5: A resealable reaction vial was charged with N-(2,2-dimethoxyethyl)-3-(methoxymethyl)-1H-pyrazole-5-carboxamide (1.4 g, 1 Eq, 5.8 mmol), and HCl (5 M) (25 mL) and a stirbar were added before being evacuated and purged with nitrogen three times. The mixture was stirred for 3 h at 25° C. The residue was purified by reverse flash chromatography with the following conditions: column, C18 silica gel; mobile phase, MeCN in water, 10%. Concentration in vacuo resulted in 7-hydroxy-2-(methoxymethyl)-6,7-dihydropyrazolo[1,5-a]pyrazin-4(5H)-one (1 g, 5 mmol, 90%) as a white solid.

### 2-(methoxymethyl)pyrazolo[1,5-a]pyrazin-4(5H)-one

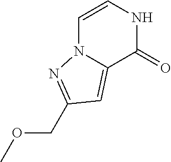

Step 6: A round bottomed flask was charged with 7-hydroxy-2-(methoxymethyl)-6,7-dihydropyrazolo[1,5-a]pyrazin-4(5H)-one (1 g, 1 Eq, 5 mmol), yoluene (20 mL) and a stirbar, and 4A-Ms (0.2 g, 1 Eq, 5 mmol), DMF (0.04 g, 0.04 mL, 0.1 Eq, 0.5 mmol), and SOCl₂ (1 g, 0.7 mL, 2 Eq, 0.01 mol) were added at 0° C., and the solution was stirred at 80° C. for 2 hours. The reaction mixture was filtered through a pad of Celite, the pad was washed with ACN, and the filtrate was concentrated in vacuo. The combined organic layers were washed with brine, dried over sodium sulfate, filtered, and concentrated to afford 2-(methoxymethyl)pyrazolo[1,5-a]pyrazin-4(5H)-one (840 mg, 4.69 mmol, 90%) as a yellow amorphous solid.

### 4-chloro-2-(methoxymethyl)pyrazolo[1,5-a]pyrazine

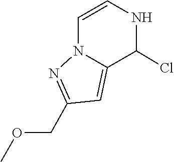

Step 7: A round bottomed flask was charged with 2-(methoxymethyl)pyrazolo[1,5-a]pyrazin-4(5H)-one (840 mg, 1 Eq, 4.69 mmol), POCl₃ (15 mL), and a stirbar, and DMF (34.3 mg, 36.3 μL, 0.1 Eq, 469 μmol) was added. The solution was stirred at 50° C. for 16 hours. The reaction mixture was quenched with water. The resulting crude material was purified by Flash (acetonitrile/water). Concentration in vacuo afforded 4-chloro-2-(methoxymethyl)pyrazolo[1,5-a]pyrazine (600 mg, 3.04 mmol, 64.8%) as a blown amorphous solid.

### Example 27

### rel-(3R,5R)-5-(3-((2-(methoxymethyl)pyrazolo[1,5-a]pyrazin-4-yl)amino)-1H-pyrazol-5-yl)tetrahydrofuran-3-yl isopropylcarbamate and

### rel-(3R,5R)-5-(3-((2-(methoxymethyl)pyrazolo[1,5-a]pyrazin-4-yl)amino)-1H-pyrazol-5-yl)tetrahydrofuran-3-yl isopropylcarbamate

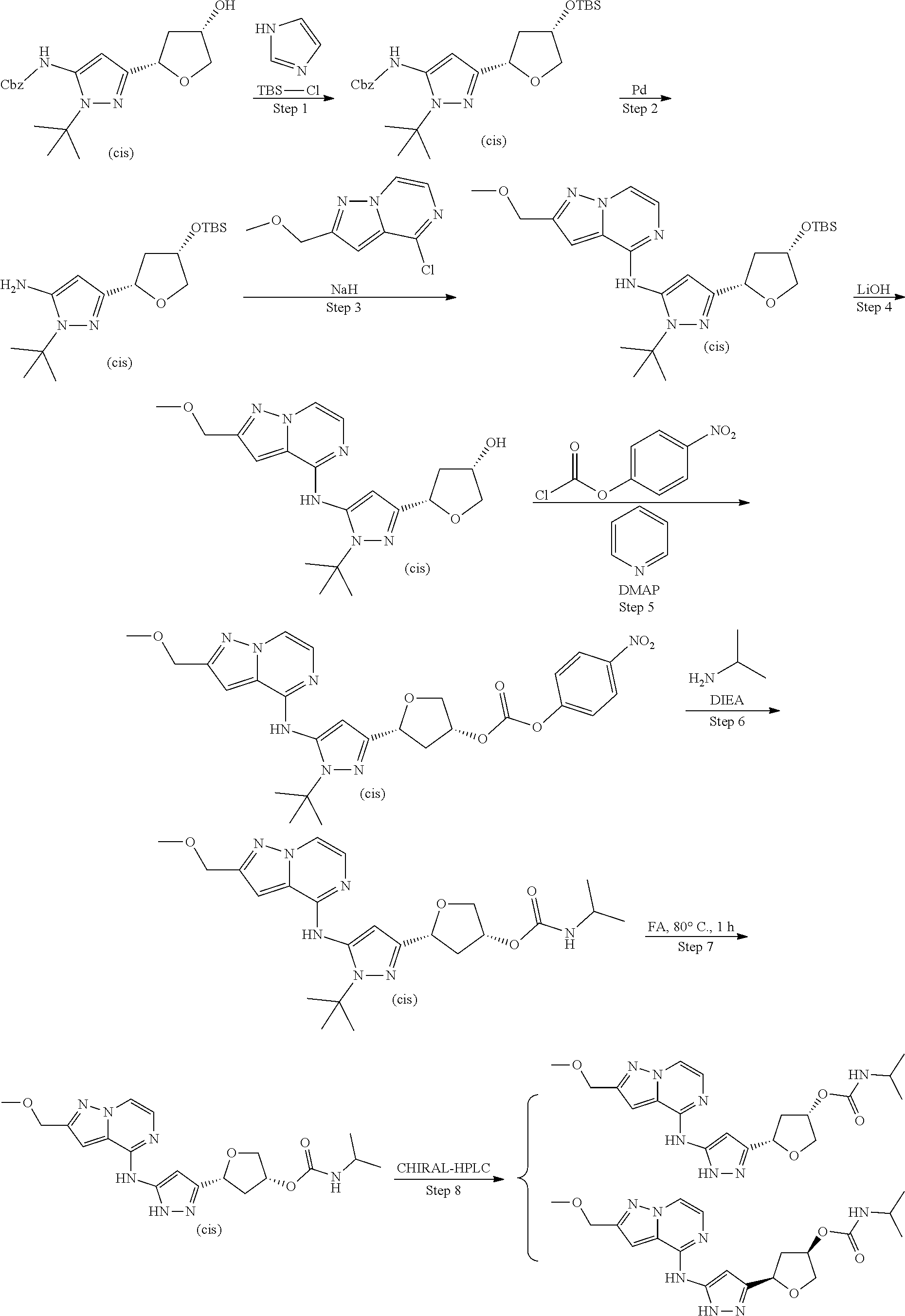

### benzyl (1-(tert-butyl)-3-((2S,4S)-4-((tert-butyldimethylsilyl)oxy)tetrahydrofuran-2-yl)-1H-pyrazol-5-yl)carbamate

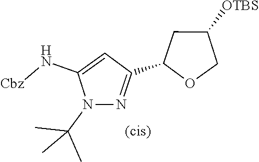 [SMILES: CC(C)(C)n1nc([C@@H]2C[C@H](O[Si](C)(C)C(C)(C)C)CO2)cc1NC(=O)OCc1ccccc1]

Step 1: A round bottomed flask was charged with benzyl (1-(tert-butyl)-3-((2S,4S)-4-hydroxytetrahydrofuran-2-yl)-1H-pyrazol-5-yl)carbamate (1000 mg, 1 Eq, 2.782 mmol), imidazole (568.2 mg, 3 Eq, 8.347 mmol),T BS—Cl (1.048 g, 2.5 Eq, 6.955 mmol) and a stirbar. DMF (10 mL) was added, and the solution was stirred for 5 hour at 25° C. LC/MS showed the reaction was completed. The mixture was quenched with water. The reaction mixture was diluted with water (30 mL), and the aqueous phase was extracted with EA (80 mL) three times. The combined organic layers were washed with brine, dried over sodium sulfate, filtered, and concentrated in vacuo resulting in benzyl (1-(tert-butyl)-3-((2S,4S)-4-((tert-butyldimethylsilyl)oxy)tetrahydrofuran-2-yl)-1H-pyrazol-5-yl)carbamate (1.2 g, 2.0 mmol, 71%, 78% Purity) as a light yellow oil.

### 1-(tert-butyl)-3-((2S,4S)-4-((tertbutyldimethylsilyl)oxy)tetrahydrofuran-2-yl)-1H-pyrazol-5-amine

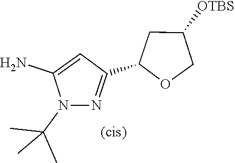 [SMILES: CC(C)(C)n1nc([C@@H]2C[C@H](O[Si](C)(C)C(C)(C)C)CO2)cc1N]

Step 2: A stirred mixture of benzyl (1-(tert-butyl)-3-((2S,4S)-4-((tert-butyldimethylsilyl)oxy)tetrahydrofuran-2-yl)-1H-pyrazol-5-yl)carbamate (1.2 g, 1 Eq, 2.5 mmol) and Pd/C (0.11 g, 0.4 Eq, 1.0 mmol) in THE (10 mL) and EA (10 mL) was treated with H₂ for 2 h at 25° C. LC/MS showed the reaction was completed. The reaction mixture was filtered, the solid was washed with ACN, and the filtrate was concentrated in vacuo. The residue was purified by reverse flash chromatography with the following conditions: column, C18 silica gel; mobile phase, MeCN in water, 30% to 70% gradient in 10 min; detector, UV 254 nm. concentrated in vacuo resulted in 1-(tert-butyl)-3-((2S,4S)-4-((tertbutyldimethylsilyl)oxy)tetrahydrofuran-2-yl)-1H-pyrazol-5-amine (850 mg, 2.4 mmol, 96%, 97% Purity) as a yellow oil.

### N-(1-(tert-butyl)-3-((2S,4S)-4-((tert-butyldimethylsilyl)oxy)tetrahydrofuran-2-yl)-1H-pyrazol-5-yl)-2-(methoxymethyl)pyrazolo[1,5-a]pyrazin-4-amine

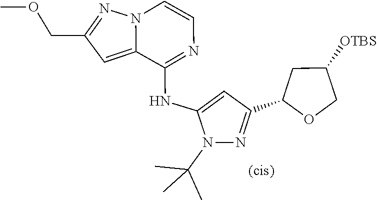 [SMILES: COCc1cc2c(Nc3cc([C@@H]4C[C@H](O[Si](C)(C)C(C)(C)C)CO4)nn3C(C)(C)C)nccn2n1]

Step 3: To a stirred solution of 1-(tert-butyl)-3-((2S,4S)-4-((tert-butyldimethylsilyl)oxy)tetrahydrofuran-2-yl)-1H-pyrazol-5-amine (200 mg, 1 Eq, 589 μmol) in DMF (5 mL) were added 4A-Ms and NaH (141 mg, 60% Wt, 6 Eq, 3.53 mmol) under 0° C. The reaction was stirred at 0° C. for 0.5 h. To the above reaction was added 4-chloro-2-(methoxymethyl)pyrazolo[1,5-a]pyrazine (140 mg, 1.2 Eq, 707 μmol) in DMF under N₂. The reaction was stirred at room temperature for 3 h. LC/MS confirmed reaction was complete. The mixture was quenched with NH₄Cl (aq.) and extracted with EA (15 mL) three times. The combined organic layers were washed with brine, dried over sodium sulfate, and concentrated in vacuo. The residue was purified by reverse flash chromatography with the following conditions: column, C18 silica gel; mobile phase, ACN in water, 60% gradient in 15 min; detector, UV 254 nm to obtain N-(1-(tert-butyl)-3-((2S,4S)-4-((tert-butyldimethylsilyl)oxy)tetrahydrofuran-2-yl)-1H-pyrazol-5-yl)-2-(methoxymethyl)pyrazolo[1,5-a]pyrazin-4-amine (210 mg, 0.39 mmol, 67%, 94% Purity) as a light yellow oil.

### (3S,5S)-5-(1-(tert-butyl)-5-((2-(methoxymethyl)pyrazolo[1,5-a]pyrazin-4-yl)amino)-1H-pyrazol-3-yl)tetrahydrofuran-3-ol

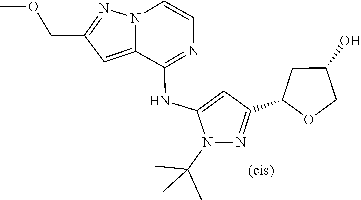 [SMILES: COCc1cc2c(Nc3cc([C@@H]4C[C@H](O)CO4)nn3C(C)(C)C)nccn2n1]

Step 4: A round bottomed flask was charged with N-(1-(tert-butyl)-3-((2S,4S)-4-((tert-butyldimethylsilyl)oxy)tetrahydrofuran-2-yl)-1H-pyrazol-5-yl)-2-(methoxymethyl)pyrazolo[1,5-a]pyrazin-4-amine (210 mg, 1 Eq, 419 μmol) and a stirbar. FA (4 mL) was added, and the solution was stirred for 2 h at 25° C. LC/MS showed the reaction was completed. The resulting mixture was concentrated in vacuo and used in the next step directly. To a stirred solution of the residue in MeOH (4 mL) and water (1 mL) was added LiOH (100 mg, 10 Eq, 4.19 mmol). The reaction was stirred at room temperature for 1 h. LC/MS showed the reaction was completed. The resulting mixture was concentrated in vacuo. The residue was purified by reverse flash chromatography with the following conditions: column, C18 silica gel; mobile phase, MeCN in water, 10% to 40% gradient in 10 min; detector, UV 254 nm. concentrated in vacuo resulting in (3S,5S)-5-(1-(tert-butyl)-5-((2-(methoxymethyl)pyrazolo[1,5-a]pyrazin-4-yl)amino)-1H-pyrazol-3-yl)tetrahydrofuran-3-ol (130 mg, 0.33 mmol, 79%, 98% Purity) as a light yellow solid.

### (3R,5R)-5-(1-(tert-butyl)-5-((2-(methoxymethyl)pyrazolo[1,5-a]pyrazin-4-yl)amino)-1H-pyrazol-3-yl)tetrahydrofuran-3-yl (4-nitrophenyl) carbonate

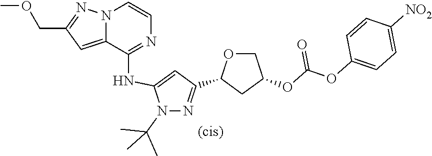 [SMILES: COCc1cc2c(Nc3cc([C@H]4C[C@@H](OC(=O)Oc5ccc([N+](=O)[O-])cc5)CO4)nn3C(C)(C)C)nccn2n1]

Step 5: To a stirred solution of (3S,5S)-5-(1-(tert-butyl)-5-((2-(methoxymethyl)pyrazolo[1,5-a]pyrazin-4-yl)amino)-1H-pyrazol-3-yl)tetrahydrofuran-3-ol (125 mg, 1 Eq, 323 μmol) in DCM (4 mL) were added pyridine (76.8 mg, 78.5 μL, 3 Eq, 970 μmol) and DMAP (3.95 mg, 0.1 Eq, 32.3 μmol) under 0° C. To the above reaction was added 4-nitrophenyl carbonochloridate (97.8 mg, 1.5 Eq, 485 μmol) in DCM under N₂. The reaction was stirred at room temperature for 16 h. LC/MS showed the reaction was completed. The resulting mixture was concentrated in vacuo. The residue was purified by reverse flash chromatography with the following conditions: column, C18 silica gel; mobile phase, ACN in water, 10% to 50% gradient in 10 min; detector, UV 254 nm. concentrated in vacuo to afford (3R,5R)-5-(1-(tert-butyl)-5-((2-(methoxymethyl)pyrazolo[1,5-a]pyrazin-4-yl)amino)-1H-pyrazol-3-yl)tetrahydrofuran-3-yl (4-nitrophenyl) carbonate (64 mg, 0.11 mmol, 33%, 93% Purity) as a light yellow solid.

### (3R,5R)-5-(1-(tert-butyl)-5-((2-(methoxymethyl)pyrazolo[1,5-a]pyrazin-4-yl)amino)-1H-pyrazol-3-yl)tetrahydrofuran-3-yl isopropylcarbamate

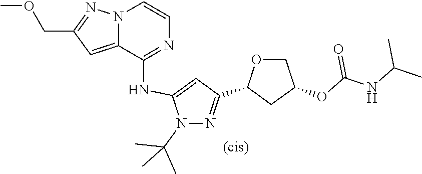 [SMILES: COCc1cc2c(Nc3cc([C@H]4C[C@@H](OC(=O)NC(C)C)CO4)nn3C(C)(C)C)nccn2n1]

Step 6: A round bottomed flask was charged with (3R,5R)-5-(1-(tert-butyl)-5-((2-(methoxymethyl)pyrazolo[1,5-a]pyrazin-4-yl)amino)-1H-pyrazol-3-yl)tetrahydrofuran-3-yl (4-nitrophenyl)carbonate (60 mg, 1 Eq, 0.11 mmol), DIEA (42 mg, 57 μL, 3 Eq, 0.33 mmol), propan-2-amine (9.6 mg, 1.5 Eq, 0.16 mmol) and a stirbar. 2Me-THF (3 mL) was added, and the solution was stirred for 16 h at 25° C. under nitrogen atmosphere. LC/MS showed the reaction was completed. The resulting mixture was concentrated in vacuo. The residue was purified by reverse flash chromatography with the following conditions: column, C18 silica gel; mobile phase, MeCN in water, 20% to 60% gradient in 10 min; detector, UV 254 nm. concentrated in vacuo to afford (3R,5R)-5-(1-(tert-butyl)-5-((2-(methoxymethyl)pyrazolo[1,5-a]pyrazin-4-yl)amino)-1H-pyrazol-3-yl)tetrahydrofuran-3-yl isopropylcarbamate (42 mg, 85 u mol, 78%, 95% Purity) as a white oil.

### (3R,5R)-5-(5-((2-(methoxymethyl)pyrazolo[1,5-a]pyrazin-4-yl)amino)-1H-pyrazol-3-yl)tetrahydrofuran-3-yl isopropylcarbamate

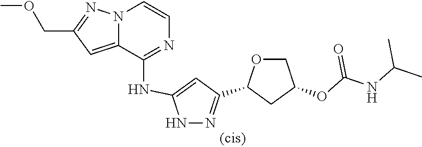 [SMILES: COCc1cc2c(Nc3cc([C@H]4C[C@@H](OC(=O)NC(C)C)CO4)n[nH]3)nccn2n1]

Step 7: A round bottomed flask was charged with (3R,5R)-5-(1-(tert-butyl)-5-((2-(methoxymethyl)pyrazolo[1,5-a]pyrazin-4-yl)amino)-1H-pyrazol-3-yl)tetrahydrofuran-3-yl isopropylcarbamate (40 mg, 1 Eq, 85 μmol) and a stirbar. FA (2 mL) was added, and the solution was stirred for 1 h at 80° C. LC/MS showed the reaction was completed. The mixture was allowed to cool down to 25° C. The resulting mixture was concentrated in vacuo. The residue was purified by reverse flash chromatography with the following conditions: column, C18 silica gel; mobile phase, MeCN in water, 10% to 50% gradient in 10 min; detector, UV 254 nm. concentrated in vacuo resulting in (3R,5R)-5-(5-((2-(methoxymethyl)pyrazolo[1,5-a]pyrazin-4-yl)amino)-1H-pyrazol-3-yl)tetrahydrofuran-3-yl isopropylcarbamate (30 mg, 69 μmol, 81%, 95% Purity) as a white solid.

### rel-(3R,5R)-5-(3-((2-(methoxymethyl)pyrazolo[1,5-a]pyrazin-4-yl)amino)-1H-pyrazol-5-yl)tetrahydrofuran-3-yl isopropylcarbamate

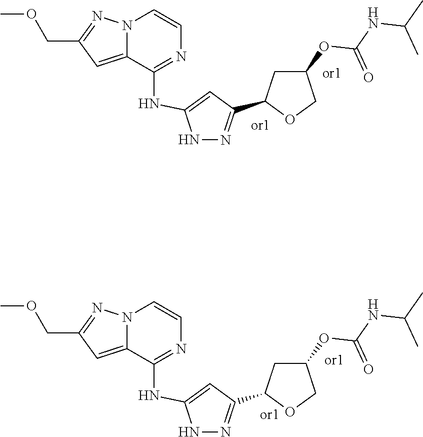

Step 8: (3R,5R)-5-(5-((2-(methoxymethyl)pyrazolo[1,5-a]pyrazin-4-yl)amino)-1H-pyrazol-3-yl)tetrahydrofuran-3-yl isopropylcarbamate (30 mg, 1 Eq, 72 μmol) was separated by chiral separation (HPLC) with the following conditions (Column: DZ-CHIRALPAK IG-3, 4.6*50 mm, 3.0 μm; Mobile Phase A: Hex(0.2% DEA): (MeOH: DCM=1:1)=60:40; Flow rate: 1 mL/min; Gradient: 0% B to 0% B; Injection Volume: 5 μl mL) to afford rel-(3R,5R)-5-(3-((2-(methoxymethyl)pyrazolo[1,5-a]pyrazin-4-yl)amino)-1H-pyrazol-5-yl)tetrahydrofuran-3-yl isopropylcarbamate (8.2 mg, 20 μmol, 54%, 99.2% Purity) as a off-white solid.

### Example 28

### (3R,5R)-5-(5-amino-1-(tert-butyl)-1H-pyrazol-3-yl)tetrahydrofuran-3-yl (1-methylcyclopropyl)carbamate

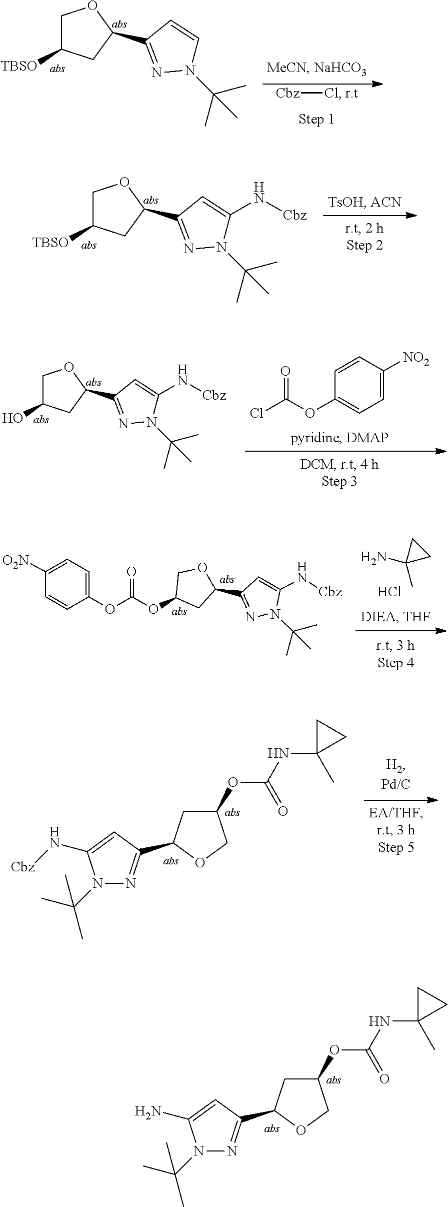

### benzyl (1-(tert-butyl)-3-((2R,4R)-4-((tert-butyldimethylsilyl)oxy)tetrahydrofuran-2-yl)-1H-pyrazol-5-yl)carbamate

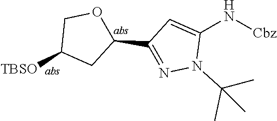 [SMILES: CC(C)(C)n1nc([C@H]2C[C@@H](O[Si](C)(C)C(C)(C)C)CO2)cc1NC(=O)OCc1ccccc1]

Step 1: A round bottomed flask was charged with 1-(tert-butyl)-3-((2R,4R)-4-((tert-butyldimethylsilyl)oxy)tetrahydrofuran-2-yl)-1H-pyrazol-5-amine (9.56 g, 28.2 mmol), NaHCO₃(11.8 g 141 mmol), MeCN (100 mL) and a stirbar. Then benzyl carbonochloridate (14.4 g, 84.5 mmol) was added at 0° C. The solution was stirred at 25° C. for 16 hours. Concentration in vacuum. The mixture was diluted with water (150 mL), and the aqueous phase was extracted with EA (3*150 mL). The combined organic layers were washed with brine, dried over sodium sulfate, filtered. Concentration in vacuum resulted in benzyl (1-(tert-butyl)-3-((2R,4R)-4-((tert-butyldimethylsilyl)oxy)tetrahydrofuran-2-yl)-1H-pyrazol-5-yl)carbamate (22 g, 23 mmol, 82%, 50% Purity) (crude) as a yellow oil. m/z (ES⁺) [M+H]⁺=474.40; HPLC tR=1.237 min.

### benzyl (1-(tert-butyl)-3-((2R,4R)-4-hydroxytetrahydrofuran-2-yl)-1H-pyrazol-5-yl)carbamate

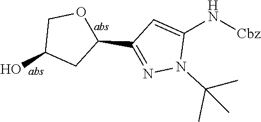 [SMILES: CC(C)(C)n1nc([C@H]2C[C@@H](O)CO2)cc1NC(=O)OCc1ccccc1]

Step 2: A round bottomed flask was charged with benzyl (1-(tert-butyl)-3-((2R,4R)-4-((tert-butyldimethylsilyl)oxy)tetrahydrofuran-2-yl)-1H-pyrazol-5-yl)carbamate (22 g, 50% Purity, 28 mmol), 4-methylbenzenesulfonic acid (14 g, 84 mmol) in ACN (200 mL) and a stirbar. The solution was stirred for 2 hours at 25° C. LCMS OK. The resulting mixture was concentrated under vacuum. The mixture was neutralized to ˜ pH 7. The reaction mixture was diluted with water (100 mL), and the aqueous phase was extracted with EA (200 mL) three times. The combined organic layers were washed with brine, dried over sodium sulfate, filtered, and concentrated in vacuum. The crude product was recrystallized from EA (100 ml) to afford benzyl (1-(tert-butyl)-3-((2R,4R)-4-hydroxytetrahydrofuran-2-yl)-1H-pyrazol-5-yl)carbamate (8.51 g, 23.7 mmol, 85%) as a white solid. m/z (ES⁺) [M+H]⁺=360.35; HPLC tR=0.760 min.

### benzyl (1-(tert-butyl)-3-((2R,4R)-4-(((4-nitrophenoxy)carbonyl)oxy)tetrahydrofuran-2-yl)-1H-pyrazol-5-yl)carbamate

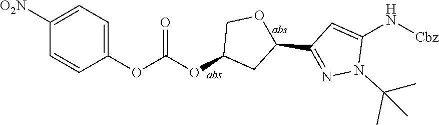 [SMILES: CC(C)(C)n1nc([C@H]2C[C@@H](OC(=O)Oc3ccc([N+](=O)[O-])cc3)CO2)cc1NC(=O)OCc1ccccc1]

Step 3: To a stirred solution of benzyl (1-(tert-butyl)-3-((2R,4R)-4-hydroxytetrahydrofuran-2-yl)-1H-pyrazol-5-yl)carbamate (4.45 g, 12.4 mmol) in DCM (40 mL) was added pyridine (2.94 g, 37.1 mmol) and N,N-dimethylpyridin-4-amine (303 mg, 2.48 mmol) under 0° C. To above reaction was added 4-nitrophenyl carbonochloridate (3.74 g, 18.6 mmol) under N₂. The reaction was stirred at 25° C. for 4 hours. The resulting mixture was concentrated under vacuum to afford a crude benzyl (1-(tert-butyl)-3-((2R,4R)-4-(((4-nitrophenoxy)carbonyl)oxy)tetrahydrofuran-2-yl)-1H-pyrazol-5-yl)carbamate (6.2 g, 5.9 mmol, 48%, 50% Purity) as a light yellow oil. m/z (ES⁺) [M+H]⁺=525.30; HPLC tR=1.020 min.

### (3R,5R)-5-(5-(((benzyloxy)carbonyl)amino)-1-(tert-butyl)-1H-pyrazol-3-yl)tetrahydrofuran-3-yl (1-methylcyclopropyl)carbamate

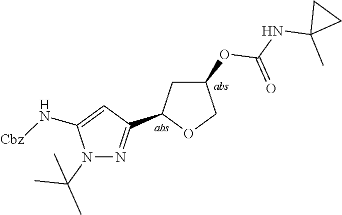 [SMILES: CC1(NC(=O)O[C@H]2CO[C@@H](c3cc(NC(=O)OCc4ccccc4)n(C(C)(C)C)n3)C2)CC1]

Step 4: A round bottomed flask was charged with benzyl (1-(tert-butyl)-3-((2R,4R)-4-(((4-nitrophenoxy)carbonyl)oxy)tetrahydrofuran-2-yl)-1H-pyrazol-5-yl)carbamate (6.2 g, 12 mmol), 1-methylcyclopropan-1-amine hydrochloride (2.5 g, 24 mmol), THE (60 mL), DIEA (6.1 g, 8.2 mL, 47 mmol) and a stirbar. The solution was stirred for 3 hours at 25° C. under nitrogen atmosphere. The resulting mixture was concentrated under vacuum. The residue was purified by reverse flash chromatography with the following conditions: column, C18 silica gel; mobile phase, MeCN in water, 10% to 50% gradient in 10 min; detector, UV 254 nm. concentrated in vacuo resulted in (3R,5R)-5-(5-(((benzyloxy)carbonyl)amino)-1-(tert-butyl)-1H-pyrazol-3-yl)tetrahydrofuran-3-yl (1-methylcyclopropyl)carbamate (4.6 g, 10 mmol, 85%) as a white oil. m/z (ES⁺) [M+H]⁺=457.35; HPLC tR=0.694 min.

### (3R,5R)-5-(5-amino-1-(tert-butyl)-1H-pyrazol-3-yl)tetrahydrofuran-3-yl (1-methylcyclopropyl)carbamate

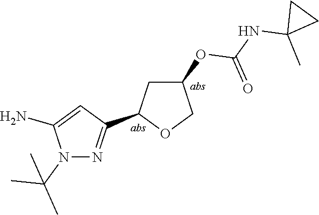 [SMILES: CC1(NC(=O)O[C@H]2CO[C@@H](c3cc(N)n(C(C)(C)C)n3)C2)CC1]

Step 5: A solution of (3R,5R)-5-(5-(((benzyloxy)carbonyl)amino)-1-(tert-butyl)-1H-pyrazol-3-yl)tetrahydrofuran-3-yl (1-methylcyclopropyl)carbamate (9.2 g, 20 mmol) in EA/THF=2:1 (90 mL) was bubbling nitrogen through the reaction mixture for 3 times. Then Pd/C (920 mg) was added. After bubbling H₂ through the reaction mixture for 3 times. The mixture was stirred at room temperature for 3 hours with H₂. The mixture was evaporated and (3R,5R)-5-(5-amino-1-(tert-butyl)-1H-pyrazol-3-yl)tetrahydrofuran-3-yl (1-methylcyclopropyl)carbamate (6.4 g, 19 mmol, 94%, 95% Purity) was obtained as an white solid without purification for next step directly. m/z (ES⁺) [M+H]⁺=323.35; HPLC tR=0.528 min.

### Example 29

### (3R,5R)-5-(3-(3-((difluoromethoxy)methyl)-1-methyl-1H-pyrazole-5-carboxamido)-1H-pyrazol-5-yl)tetrahydrofuran-3-yl (1-methylcyclopropyl)carbamate

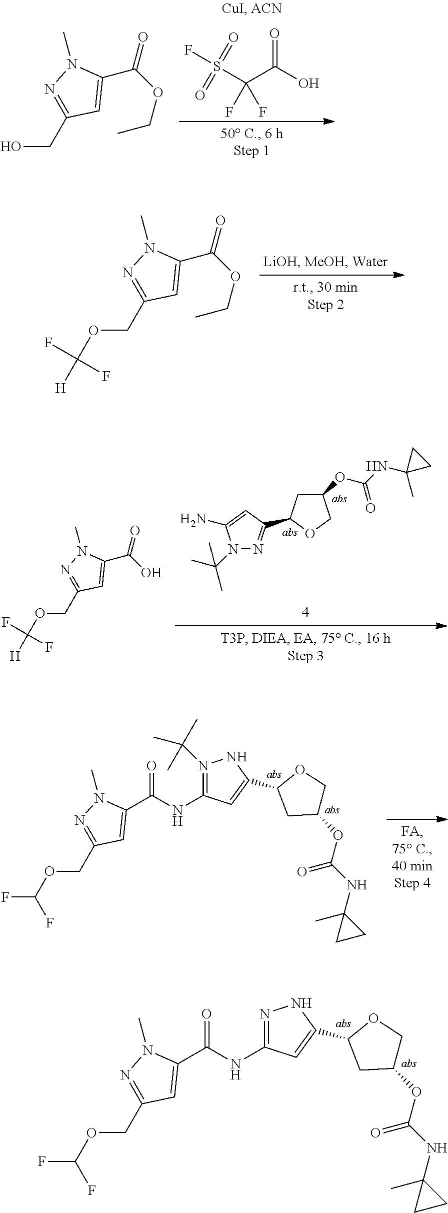

### ethyl 3-((difluoromethoxy)methyl)-1-methyl-1H-pyrazole-5-carboxylate

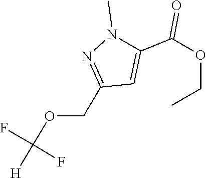 [SMILES: CCOC(=O)c1cc(COC(F)F)nn1C]

Step 1. To a stirred solution of ethyl 3-(hydroxymethyl)-1-methyl-1H-pyrazole-5-carboxylate (1 g, 5 mmol) in MeCN (10 mL) was added CuI (0.2 g, 1 mmol) at room temperature under nitrogen atmosphere. The mixture was heated to 50° C. and 2,2-difluoro-2-(fluorosulfonyl)acetic acid (1 g, 8 mmol) in MeCN was added dropwise over a period of 8 hours at 50° C. The mixture was concentrated under reduced pressure. The residue was purified by reverse flash chromatography with the following conditions: column, C18 silica gel; mobile phase, MeCN in water, 10% to 90% gradient in 15 min; detector, UV 254 nm. Concentration in vacuo resulted in ethyl 3-((difluoromethoxy)methyl)-1-methyl-1H-pyrazole-5-carboxylate (700 mg, 2.99 mmol, 60%) as white solid. m/z (ES⁺) [M+H]⁺=235.00; HPLC tR=0.836 min.

### 3-((difluoromethoxy)methyl)-1-methyl-1H-pyrazole-5-carboxylic acid

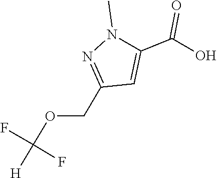

Step 2. To a stirred solution of ethyl 3-((difluoromethoxy)methyl)-1-methyl-1H-pyrazole-5-carboxylate (700 mg, 2.99 mmol) in MeOH (10 mL) was added LiOH (107 mg, 4.48 mmol) in Water (2 mL) at room temperature. The resulting mixture was stirred for 30 min at room temperature. The reaction was adjusted to ph=6 with 1N HCl. The resulting mixture was extracted with EtOAc (3×10 mL). The organic phase was concentrated under reduced pressure. The residue was purified by reverse flash chromatography with the following conditions: column, C18 silica gel; mobile phase, MeCN in water, 10% to 90% gradient in 15 min; detector, UV 254 nm. Concentration in vacuo resulted in 3-((difluoromethoxy)methyl)-1-methyl-1H-pyrazole-5-carboxylic acid (610 mg, 2.96 mmol, 99.0%) as white solid. m/z (ES⁺) [M+H]=207.10; HPLC tR=0.792 min.

### (3R,5R)-5-(1-(tert-butyl)-5-(3-((difluoromethoxy)methyl)-1-methyl-1H-pyrazole-5-carboxamido)-1H-pyrazol-3-yl)tetrahydrofuran-3-yl(1-methylcyclopropyl)carbamate

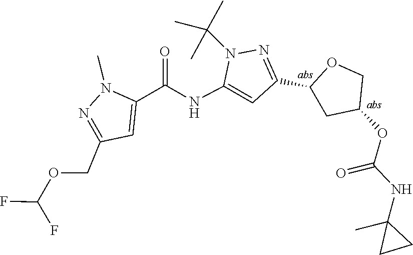 [SMILES: Cn1nc(COC(F)F)cc1C(=O)Nc1cc([C@H]2C[C@@H](OC(=O)NC3(C)CC3)CO2)nn1C(C)(C)C]

Step 3. To a stirred solution of [2170-88] 3-((difluoromethoxy)methyl)-1-methyl-1H-pyrazole-5-carboxylic acid (120 mg, 582 μmol) in EA (3 mL) was added (3R,5R)-5-(5-amino-1-(tert-butyl)-1H-pyrazol-3-1)tetrahydrofuran-3-yl (1-methylcyclopropyl)carbamate (188 mg, 582 mol), DIEA (752 mg, 5.82 mmol), T3P (2.96 g, 50% Wt in EA, 4.66 mmol) at room temperature under nitrogen atmosphere. The resulting mixture was stirred for 16 hour at 75° C. The mixture was concentrated under reduced pressure and purified by reverse flash chromatography with the following conditions: column, C18 silica gel; mobile phase, MeCN in water, 10% to 90% gradient in 15 min; detector, UV 254 nm, Concentration in vacuum resulted in (3R,5R)-5-(1-(tert-butyl)-5-(3-((difluoromethoxy)methyl)-1-methyl-1H-pyrazole-5-carboxamido)-1H-pyrazol-3-yl)tetrahydrofuran-3-yl(1-methylcyclopropyl)carbamate (130 mg, 255 μmol, 43.7%) as yellow oil. m/z (ES⁺) [M+H]⁺=511.45; HPLC tR=0.838 min.

### (3R,5R)-5-(3-(3-((difluoromethoxy)methyl)-1-methyl-1H-pyrazole-5-carboxamido)-1H-pyrazol-5-yl)tetrahydrofuran-3-yl (1-methylcyclopropyl)carbamate

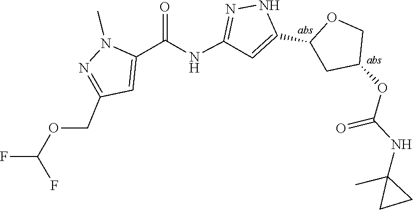 [SMILES: Cn1nc(COC(F)F)cc1C(=O)Nc1cc([C@H]2C[C@@H](OC(=O)NC3(C)CC3)CO2)[nH]n1]

Step 4. To a stirred solution of (3R,5R)-5-(1-(tert-butyl)-5-(3-((difluoromethoxy)methyl)-1-methyl-1H-pyrazole-5-carboxamido)-1Hpyrazol-3-yl)tetrahydrofuran-3-yl (1-methylcyclopropyl)carbamate (120 mg, 235 μmol) in FA (2 mL) was stirred for 40 min at 75° C. The mixture was concentrated under reduced pressure. The mixture was purified by Prep-HPLC (followed the condition:Column: XBridge Shield R^{P}18 OBD Column, 30*150 mm, 5 m; MobilePhase A: Water(10 mmol/L NH₄HCO₃), Mobile Phase B: ACN; Flowrate: 60 mL/min; Gradient: 20% B to 43% B in 8 min, 43% B; WaveLength: 220 nm; RT1(min): 7.38) to afford (3R,5R)-5-(3-(3-((difluoromethoxy)methyl)-1-methyl-1H-pyrazole-5-carboxamido)-1H-pyrazol-5-yl)tetrahydrofuran-3-yl (1-methylcyclopropyl)carbamate (24.9 mg, 54.6 μmol, 23.2%, 99.6% Purity) as white solid. m/z (ES⁺) [M+H]⁺=455.20; HPLC tR=0.893 min. ¹H NMR (400 MHz, DMSO-d₆) 12.51 (s, 1H), 10.86 (s, 1H), 7.51 (s, 1H), 7.21 (s, 1H), 6.99-6.49 (m, 2H), 5.16 (s, 1H), 4.86 (s, 3H), 4.08 (s, 3H), 3.85 (s, 2H), 2.71 (dd, J=14.0, 7.3 Hz, 1H), 1.93 (s, 1H), 1.25 (s, 3H), 0.76-0.37 (m, 4H).

### Example 30

### (3R,5R)-5-(3-(1-methyl-3-((trifluoromethoxy)methyl)-1H-pyrazole-5-carboxamido)-1H-pyrazol-5-yl)tetrahydrofuran-3-yl (1-methylcyclopropyl)carbamate

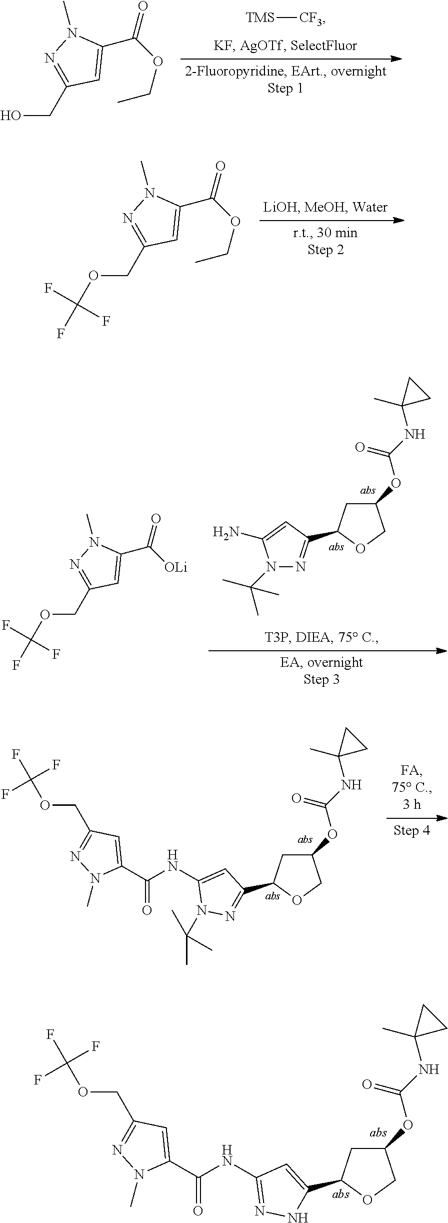

### ethyl 1-methyl-3-((trifluoromethoxy)methyl)-1H-pyrazole-5-carboxylate

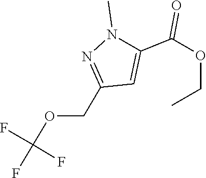 [SMILES: CCOC(=O)c1cc(COC(F)(F)F)nn1C]

Step 1. To a stirred solution of ethyl 3-(hydroxymethyl)-1-methyl-1H-pyrazole-5-carboxylate (5 g, 0.03 mol) in EtOAc (50 mL) was added potassium fluoride (5 g, 0.08 mol), Silver(I)TrifluoromethaneSulfonate (0.01 kg, 0.05 mol), Selectfluor (0.01 kg, 0.04 mol), 2-Fluoropyridin (5 g, 0.05 mol) and Trifluoromethyltrimethylsilane (8 g, 0.05 mol) at room temperature under nitrogen atmosphere. The resulting mixture was stirred for 16 hour at room temperature. The resulting mixture was filtered. The filtrate was concentrated under reduced pressure. The residue was purified by reverse flash chromatography with the following conditions: column, C18 silica gel; mobile phase, MeCN in water, 0% to 100% gradient in 15 min; detector, UV 254 nm, Concentration in vacuo resulted in ethyl 1-methyl-3-((trifluoromethoxy)methyl)-1H-pyrazole-5-carboxylate (3 g, 6 mmol, 20%, 50% Purity) as white solid. m/z (ES⁺) [M+H]⁺=253.15; HPLC tR=1.279 min.

### lithium 1-methyl-3-((trifluoromethoxy)methyl)-1H-pyrazole-5-carboxylate

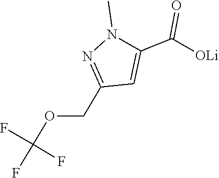

Step 2. To a stirred solution of ethyl 1-methyl-3-((trifluoromethoxy)methyl)-1H-pyrazole-5-carboxylate (3 g, 0.01 mol) in MeOH (30 mL) was added LiOH (0.4 g, 0.02 mol) in Water (12 mL) at room temperature under nitrogen atmosphere. The resulting mixture was stirred for 0.5 hour at room temperature. The mixture was concentrated under reduced pressure. The residue was purified by reverse flash chromatography with the following conditions: column, C18 silica gel; mobile phase, MeCN in water, 0% to 10% gradient in 15 min; detector, UV 254 nm, Concentration in vacuo resulted in lithium 1-methyl-3-((trifluoromethoxy)methyl)-1H-pyrazole-5-carboxylate (1.5 g, 6.7 mmol, 60%) as white solid. m/z (ES⁺) [M+H]⁺=225.10; HPLC tR=1.589 min.

### (3R,5R)-5-(1-(tert-butyl)-5-(1-methyl-3-((trifluoromethoxy)methyl)-1H-pyrazole-5-carboxamido)-1H-pyrazol-3-yl)tetrahydrofuran-3-yl (1-methylcyclopropyl)carbamate

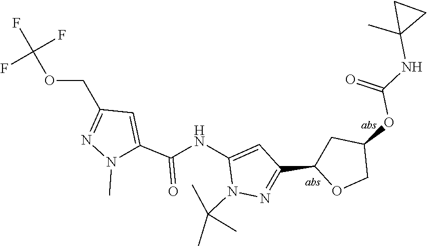 [SMILES: Cn1nc(COC(F)(F)F)cc1C(=O)Nc1cc([C@H]2C[C@@H](OC(=O)NC3(C)CC3)CO2)nn1C(C)(C)C]

Step 3. To a solution of lithium 1-methyl-3-((trifluoromethoxy)methyl)-1H-pyrazole-5-carboxylate (180 mg, 803 μmol) and (3R,5R)-5-(5-amino-1-(tert-butyl)-1H-pyrazol-3-yl)tetrahydrofuran-3-yl (1-methylcyclopropyl)carbamate (285 mg, 883 μmol) in ethyl acetate (6 mL) was added DIEA (1.04 g, 8.03 mmol). To above reaction was added T3P (4.09 g, 50% Wt in EA, 6.42 mmol) at 0° C. The reaction was stirred at 75° C. for overnight. The mixture was quenched with water, filtered and extracted with EA(3*20 ml). The combined organic layers were washed with brine, dried over sodium sulfate and concentrated. The residue was purified by reverse flash chromatography with the following conditions: column, C18 silica gel; mobile phase, MeCN in water, 60% gradient in 8 min; detector, UV 254 nm to afford (3R,5R)-5-(1-(tert-butyl)-5-(1-methyl-3-((trifluoromethoxy)methyl)-1H-pyrazole-5-carboxamido)-1H-pyrazol-3-yl)tetrahydrofuran-3-yl (1-methylcyclopropyl)carbamate (400 mg, 757 μmol, 94.2%) as a white solid. m/z (ES⁺) [M+H]^{m}=529.40; HPLC tR=1.161 min

### (3R,5R)-5-(3-(I-methyl-3-((trifluoromethoxy)methyl)₁H-pyrazole-5-carboxamido)-1H-pyrazol-5-yl)tetrahydrofuran-3-yl (1-methylcyclopropyl)carbamate

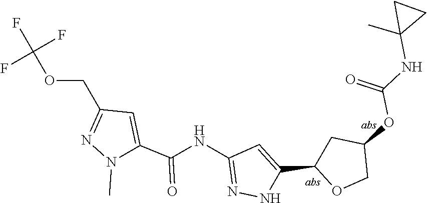 [SMILES: Cn1nc(COC(F)(F)F)cc1C(=O)Nc1cc([C@H]2C[C@@H](OC(=O)NC3(C)CC3)CO2)[nH]n1]

Step 4. The (3R,5R)-5-(1-(tert-butyl)-5-(1-methyl-3-((trifluoromethoxy)methyl)-1H-pyrazole-5-carboxamido)-1H-pyrazol-3-yl)tetrahydrofuran-3-yl (1-methylcyclopropyl)carbamate (595 mg, 1.13 mmol) was added FA (10 mL). The reaction was stirred at 75° C. for 3 h. The mixture was concentrated and purified by reverse flash chromatography with the following conditions: column, C18 silica gel; mobile phase, MeCN in water, 50% to 60% gradient in 8 min; detector, UV 254 nm. Lyophilization yielded (3,5R)-5-(3-(1-methyl-3-((trifluoromethoxy)methyl)-1H-pyrazole-5-carboxamido)-1H-pyrazol-5-yl)tetrahydrofuran-3-yl (1-methylcyclopropyl)carbamate (384.4 mg, 809 μmol, 71.8%, 99.4% Purity) as a white solid. m/z (ES⁺) [M+H]^{m}=473.10; HPLC tR=0.871 min. ¹H NMR (400 MHz, DMSO-d₆) 12.50 (s, 1H), 10.90 (s, 1H), 7.50 (s, 1H), 7.29 (s, 1H), 6.56 (s, 1H), 5.12 (s, 3H), 4.85 (s, 1H), 4.10 (s, 3H), 3.85 (s, 2H), 2.77-2.69 (m, 1H), 1.93 (s, 1H), 1.25 (s, 3H), 0.66-0.39 (m, 4H).

### Example 31

### (3R,5R)-5-(3-(1-methyl-3-((2,2,2-trifluoroethoxy)methyl)-1H-pyrazole-5-carboxamido)-1H-pyrazol-5-yl)tetrahydrofuran-3-yl (1-methylcyclopropyl)carbamate Compound 771

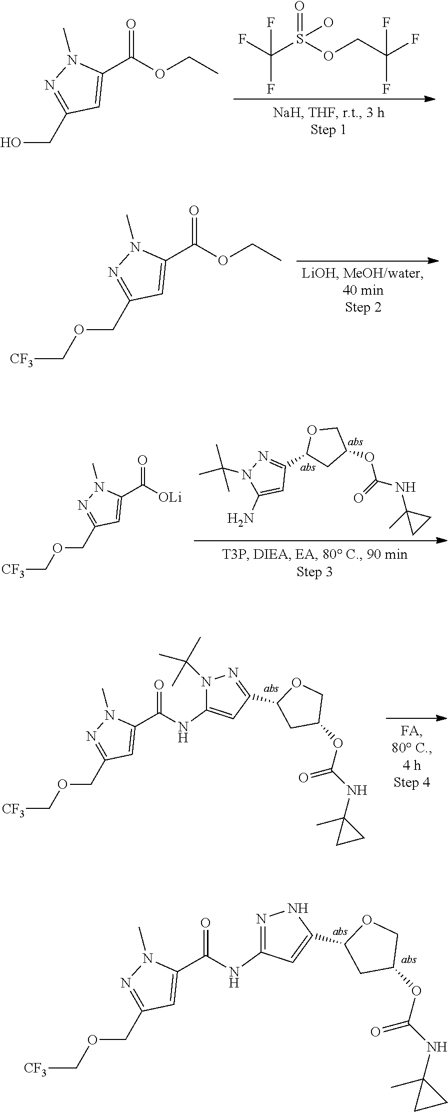

### ethyl 1-methyl-3-((2,2,2-trifluoroethoxy)methyl)-1H-pyrazole-5-carboxylate

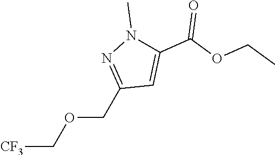 [SMILES: CCOC(=O)c1cc(COCC(F)(F)F)nn1C]

Step 1. To a stirred solution of ethyl 3-(hydroxymethyl)-1-methyl-1H-pyrazole-5-carboxylate (500 mg, 2.71 mmol) in THF (20 mL) was added 2,2,2-trifluoroethyl trifluoromethanesulfonate (1.89 g, 8.14 mmol), NaH (0.16 g, 60% Wt, 4.07 mmol) at 0° C. under nitrogen atmosphere. The resulting mixture was stirred for 3 hour at room temperature. The reaction was quenched with water. The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 90% gradient in 15 min; detector, UV 254 nm. Concentration in vacuum resulted in ethyl 1-methyl-3-((2,2,2-trifluoroethoxy)methyl)-1H-pyrazole-5-carboxylate (310 mg, 1.16 mmol, 42.9%) as yellow oil. m/z (ES⁺) [M+H]=267.25; HPLC tR=0.879 min

### 1-methyl-3-((2,2,2-trifluoroethoxy)methyl)-1H-pyrazole-5-carboxylate

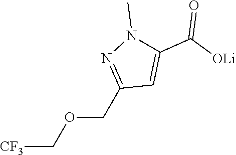

Step 2. To a stirred solution of ethyl 1-methyl-3-((2,2,2-trifluoroethoxy)methyl)-1H-pyrazole-5-carboxylate (300 mg, 1.13 mmol) in MeOH (5 mL) was added LiOH (40.5 mg, 1.69 mmol) in water (2 mL) at room temperature under nitrogen atmosphere. The resulting mixture was stirred for 40 min at room temperature. The mixture was concentrated under reduced pressure. The residue was purified by reverse flash chromatography with the following conditions: column, C18 silica gel; mobile phase, MeCN in water, 10% to 90% gradient in 15 min; detector, UV 254 nm, Concentration in vacuum resulted in lithium 1-methyl-3-((2,2,2-trifluoroethoxy)methyl)-1H-pyrazole-5-carboxylate (200 mg, 840 μmol, 74.5%) as white solid. m/z (ES⁺) [M+H]⁺=239.20; HPLC tR=0.653 min.

### (3R,5R)-5-(1-(tert-butyl)-5-(1-methyl-3-((2,2,2-trifluoroethoxy)methyl)-1H-pyrazole-5-carboxamido)-1H-pyrazol-3-yl)tetrahydrofuran-3-yl (1-methylcyclopropyl)carbamate

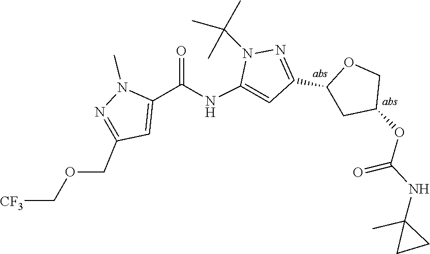 [SMILES: Cn1nc(COCC(F)(F)F)cc1C(=O)Nc1cc([C@H]2C[C@@H](OC(=O)NC3(C)CC3)CO2)nn1C(C)(C)C]

Step 3. To a stirred solution of (3R,5R)-5-(5-amino-1-(tert-butyl)-1H-pyrazol-3-yl)tetrahydrofuran-3-yl (1-methylcyclopropyl)carbamate (264 mg, 819 μmol) in EA (10 mL) was added lithium 1-methyl-3-((2,2,2-trifluoroethoxy)methyl)-1H-pyrazole-5-carboxylate (200 mg, 819 μmol), DIEA (1.06 g, 8.19 mmol), T3P (4.17 g, 50% Wt in EA, 6.55 mmol) at room temperature under nitrogen atmosphere. The resulting mixture was stirred for 90 min at 80° C. The reaction was quenched with Water. The resulting mixture was extracted with EtOAc (3×40 mL). The combined organic layers were washed with brine (1×40 mL), dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 90% gradient in 15 min; detector, UV 254 nm, Concentration in vacuo resulted in (3R,5R)-5-(1-(tert-butyl)-5-(1-methyl-3-((2,2,2-trifluoroethoxy)methyl)-1H-pyrazole-5-carboxamido)-1H-pyrazol-3-yl)tetrahydrofuran-3-yl (1-methylcyclopropyl)carbamate (250 mg, 461 μmol, 56.2%) as yellow oil. m/z (ES⁺) [M+H]⁺=543.35; HPLC tR=0.878 min.

### (3R,5R)-5-(3-(1-methyl-3-((2,2,2-trifluoroethoxy)methyl)-1H-pyrazole-5-carboxamido)-1H-pyrazol-5-yl)tetrahydrofuran-3-yl (1-methylcyclopropyl)carbamate

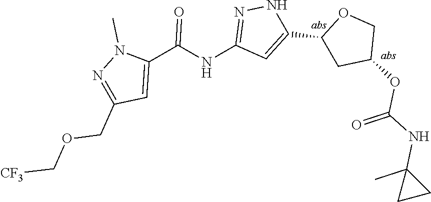 [SMILES: Cn1nc(COCC(F)(F)F)cc1C(=O)Nc1cc([C@H]2C[C@@H](OC(=O)NC3(C)CC3)CO2)[nH]n1]

Step 4. To a stirred solution of (3R,5R)-5-(1-(tert-butyl)-5-(1-methyl-3-((2,2,2-trifluoroethoxy)methyl)-1H-pyrazole-5-carboxamido)-1Hpyrazol-3-yl)tetrahydrofuran-3-yl (1-methylcyclopropyl)carbamate (245 mg, 452 μmol) in FA (5 mL) was stirred for 4 hour at 80° C. The mixture was concentrated under reduced pressure. The mixture was purified by Prep-HPLC (followed the condition:Column: XBridge Shield R^{P}18 OBD Column, 30*150 mm, 5 μm; Mobile Phase A: Water(10 mmol/L NH₄HCO₃), Mobile Phase B: ACN; Flow rate: 60 mL/min; Gradient: 25% B to 45% B in 8 min, 45% B; Wave Length: 254 nm; RT1(min): 7.12) to afford (3R,5R)-5-(3-(1-methyl-3-((2,2,2-trifluoroethoxy)methyl)-1H-pyrazole-5-carboxamido)-1H-pyrazol-5-yl)tetrahydrofuran-3-yl (1-methylcyclopropyl)carbamate (129 mg, 261 μmol, 57.7%,98.3% Purity) as white solid. m/z (ES⁺) [M+H]⁺=487.20; HPLC tR=1.399 min. ¹H NMR (400 MHz, DMSO-d₆) 12.47 (s, 1H), 10.86 (s, 1H), 7.52 (s, 1H), 7.17 (s, 1H), 6.52 (s, 1H), 5.16 (s, 1H), 4.84 (t, J=7.4 Hz, 1H), 4.61 (s, 2H), 4.14-4.05 (m, 5H), 3.84 (d, J=4.8 Hz, 2H), 2.70 (dt, J=13.6, 7.6 Hz, 1H), 1.94 (dd, J=14.9, 6.8 Hz, 1H), 1.25 (s, 3H), 0.61 (d, J=5.2 Hz, 2H), 0.48 (q, J=4.7 Hz, 2H).

### Example 32

### (3R,5R)-5-(3-(1-methyl-3-(2,2,2-trifluoroethoxy)-1H-pyrazole-5-carboxamido)-1H-pyrazol-5-yl)tetrahydrofuran-3-yl (1-methylcyclopropyl)carbamate

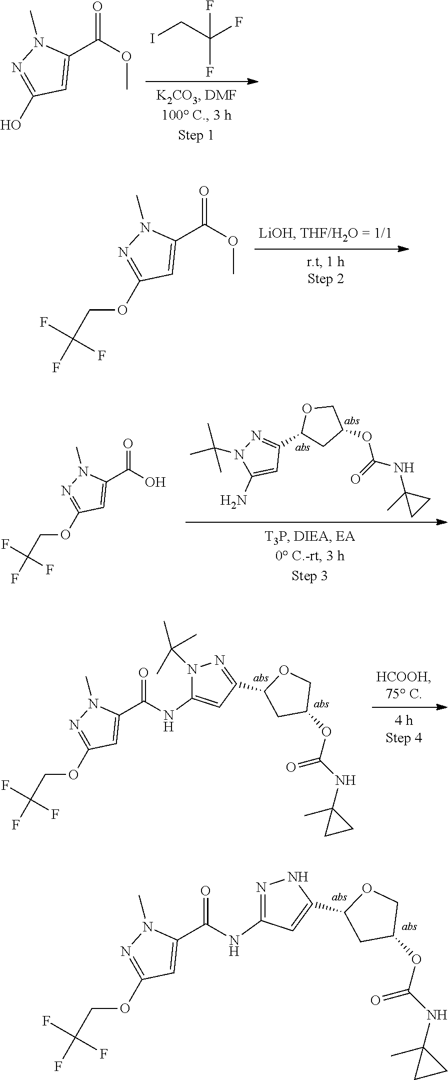

### methyl 1-methyl-3-(2,2,2-trifluoroethoxy)-1H-pyrazole-5-carboxylate

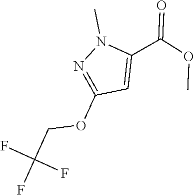 [SMILES: COC(=O)c1cc(OCC(F)(F)F)nn1C]

Step 1. A round bottomed flask was charged with methyl 3-hydroxy-1-methyl-1H-pyrazole-5-carboxylate (400 mg, 2.56 mmol), 1,1,1-trifluoro-2-iodoethane (645 mg, 3.07 mmol), K₂CO₃ (1.06 g, 7.69 mmol) and a stir bar. DMF (8 mL) was added, and the solution was stirred for 3 hours at 100° C. The reaction mixture was diluted with water (20 mL), and the aqueous phase was extracted with EA (20 mL) three times. The combined organic layers were washed with brine (20 mL) three times, dried over sodium sulfate, filtered, and concentrated in vacuum to afford methyl 1-methyl-3-(2,2,2-trifluoroethoxy)-1H-pyrazole-5-carboxylate (390 mg, 1.4 mmol, 53%, 83% Purity) as a yellow amorphous solid. m/z (ES⁺) [M+H]⁺=239.00; HPLC tR=0.875 min.

### 1-methyl-3-(2,2,2-trifluoroethoxy)-1H-pyrazole-5-carboxylic acid

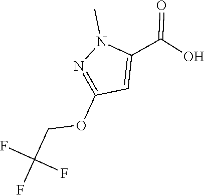

Step 2. A round bottomed flask was charged with methyl 1-methyl-3-(2,2,2-trifluoroethoxy)-1H-pyrazole-5-carboxylate (380 mg, 1.60 mmol) in THE (4 mL), and LiOH (115 mg in 4 mL H₂O, 4.79 mmol) and a stirbar. The resulting solution was stirred for 1 hour at 25° C. The mixture was adjusted pH value to 6-7. The reaction mixture was diluted with water (20 mL), and the aqueous phase was extracted with EA (20 mL) three times, dried over sodium sulfate, filtered, and concentrated in vacuum to afford 1-methyl-3-(2,2,2-trifluoroethoxy)-1H-pyrazole-5-carboxylic acid (350 mg, 1.2 mmol, 77%, 79% Purity) as a yellow amorphous solid. m/z (ES⁺) [M+H]⁺=225.25; HPLC tR=0.705 min

### (3R,5R)-5-(1-(tert-butyl)-5-(1-methyl-3-(2,2,2-trifluoroethoxy)-1H-pyrazole-5-carboxamido)-1H-pyrazol-3-yl)tetrahydrofuran-3-yl (1-methylcyclopropyl)carbamate

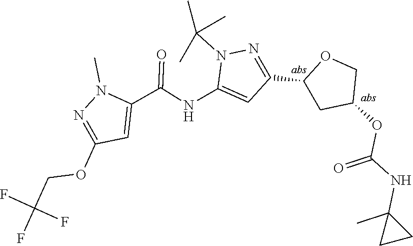 [SMILES: Cn1nc(OCC(F)(F)F)cc1C(=O)Nc1cc([C@H]2C[C@@H](OC(=O)NC3(C)CC3)CO2)nn1C(C)(C)C]

Step 3. To a mixture of (3R,5R)-5-(5-amino-1-(tert-butyl)-1H-pyrazol-3-yl)tetrahydrofuran-3-yl (1-methylcyclopropyl)carbamate (198 mg, 613 μmol), 1-methyl-3-(2,2,2-trifluoroethoxy)-1H-pyrazole-5-carboxylic acid (200 mg, 705 μmol) and DIEA (317 mg, 2.45 mmol) in EA (6 mL) was added T3P (780 mg of a solution 1 M in EA, 50% Wt, 1.23 mmol) drop wise at 0° C. under nitrogen atmosphere. The mixture was stirred for 3 hours at 25° C. The mixture was concentrated in vacuum. The residue was purified by reverse flash chromatography with the following conditions: column, C18 silica gel; mobile phase, MeCN in water, 0% to 100% gradient in 20 min; detector, UV 254 nm to afford (3R,5R)-5-(1-(tert-butyl)-5-(1-methyl-3-(2,2,2-trifluoroethoxy)-1H-pyrazole-5-carboxamido)-1H-pyrazol-3-yl)tetrahydrofuran-3-yl (1-methylcyclopropyl)carbamate (240 mg, 454 μmol, 74.1%) as a clear amorphous solid. m/z (ES⁺) [M+H]⁺=529.10; HPLC tR=0.931 min.

### (3R,5R)-5-(3-(1-methyl-3-(2,2,2-trifluoroethoxy)-1H-pyrazole-5-carboxamido)-1H-pyrazol-5-yl)tetrahydrofuran-3-yl (1-methylcyclopropyl)carbamate

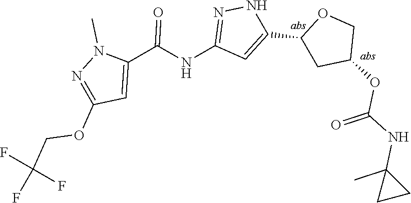 [SMILES: Cn1nc(OCC(F)(F)F)cc1C(=O)Nc1cc([C@H]2C[C@@H](OC(=O)NC3(C)CC3)CO2)[nH]n1]

Step 4. A round bottomed flask was charged with (3R,5R)-5-(1-(tert-butyl)-5-(1-methyl-3-(2,2,2-trifluoroethoxy)-1H-pyrazole-5-carboxamido)-1H-pyrazol-3-yl)tetrahydrofuran-3-yl (1-methylcyclopropyl)carbamate (235 mg, 445 μmol) and a stirbar. FA (7 mL) was added, and the solution was stirred for 4 hours at 75° C. The mixture was concentrated in vacuum. The resulting crude material was purified by Pre-HPLC(Column: XBridge Shield R^{P}18 OBD Column, 30*150 mm, 5 m; Mobile Phase A: Water(10 mmol/L NH₄HCO₃), Mobile Phase B: ACN; Flow rate: 60 mL/min; Gradient: 28% B to 48% B in 8 min, 48% B; Wave Length: 254 nm; RT1(min): 7.38). Lyophilization yielded (3R,5R)-5-(3-(1-methyl-3-(2,2,2-trifluoroethoxy)-1H-pyrazole-5-carboxamido)-1H-pyrazol-5-yl)tetrahydrofuran-3-yl (1-methylcyclopropyl)carbamate (183.7 mg, 388.8 μmol, 87.5%) as a white solid. m/z (ES⁺) [M+H]+=473.05; HPLC tR=1.018 min. ¹H NMR (400 MHz, DMSO-d₆) 12.53 (s, 1H), 10.82 (s, 1H), 7.50 (s, 1H), 6.62 (d, J=48.9 Hz, 2H), 5.16 (s, 1H), 4.80 (q, J=8.9 Hz, 3H), 3.97 (s, 3H), 3.85 (s, 2H), 2.70 (dt, J=13.1, 6.9 Hz, 1H), 1.93 (s, 1H), 1.25 (s, 3H), 0.65-0.37 (m, 4H).

### Example 33

### (3R,5R)-5-(3-(imidazo[1,2-c] pyrimidin-5-ylamino)-1H-pyrazol-5-yl) tetrahydrofuran-3-yl (1-methylcyclopropyl) carbamate

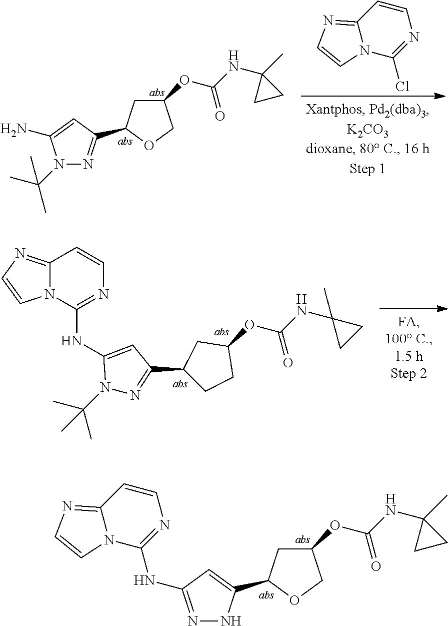

### (3R,5R)-5-(1-(tert-butyl)-5-(imidazo[1,2-c]pyrimidin-5-ylamino)-1H-pyrazol-3-yl)tetrahydrofuran-3-yl (1-methylcyclopropyl)carbamate

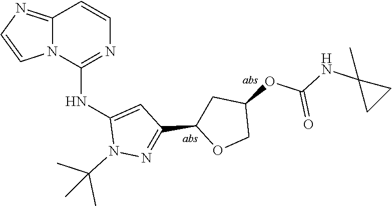 [SMILES: CC1(NC(=O)O[C@H]2CO[C@@H](c3cc(Nc4nccc5nccn45)n(C(C)(C)C)n3)C2)CC1]

Step 1. 5-chloroimidazo[1,2-c] pyrimidine (57.2 mg, 1.2 Eq, 372 μmol), K₂CO₃ (129 mg, 3 Eq, 930 μmol) and xantphos (35.9 mg, 0.2 Eq, 62.0 μmol) were added to a solution of (3R,5R)-5-(3-amino-1-(tert-butyl)-1H-pyrazol-5-yl) tetrahydrofuran-3-yl (1-methylcyclopropyl) carbamate (100 mg, 1 Eq, 310 μmol) in 1,4-Dioxane (5 mL). After bubbling nitrogen through the reaction mixture for 1 minutes, Pd₂(dba)₃ (56.8 mg, 0.2 Eq, 62.0 μmol) was added. The reaction mixture was heated at 80° C. for 16 hours with vigorous stirring. After cooling, the reaction was worked up by addition of 15 mL water and extraction with ethyl acetate, dried over Na₂SO₄ and evaporated in vacuo. The crude residue was purified by flash (Mobile Phase A: Water (10 mmol/L NH₄HCO₃), Mobile Phase B: ACN; Flow rate: 35 mL/min; Gradient: 30% B to 60% B in 15 min); After solvent evaporation afforded the title compound (3R,5R)-5-(1-(tert-butyl)-5-(imidazo[1,2-c]pyrimidin-5-ylamino)-1H-pyrazol-3-yl)tetrahydrofuran-3-yl (1-methylcyclopropyl)carbamate (70 mg, 0.16 mmol, 51%) as a yellow solid. m/z (ES⁺) [M+H]=440.40; HPLC tR=0.592 min

### (3R,5R)-5-(3-(imidazo[1,2-c] pyrimidin-5-ylamino)-1H-pyrazol-5-yl) tetrahydrofuran-3-yl (1-methylcyclopropyl) carbamate

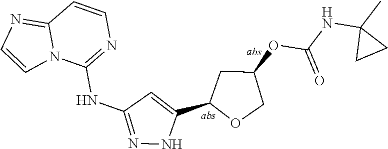 [SMILES: CC1(NC(=O)O[C@H]2CO[C@@H](c3cc(Nc4nccc5nccn45)n[nH]3)C2)CC1]

Step 2. The solution of (3R,5R)-5-(1-(tert-butyl)-5-(imidazo[1,2-c] pyrimidin-5-ylamino)-1H-pyrazol-3-yl) tetrahydrofuran-3-yl (1-methylcyclopropyl) carbamate (50 mg, 1 Eq, 0.11 mmol) in FA (7.5 mL) was divided into five parts on average. The five mixtures were heated under 100° C. for 1.5 hour. Product could be found in LCMS. Combine 5 batch together for purification. After cooling, the reaction was evaporated in vacuo. The crude residue was purified by Prep-HPLC (Column: YMC-Actus Triart C18 ExRS, 30*150 mm, 5 m; Mobile Phase A: Water (10 mmol/L NH₄HCO₃), Mobile Phase B: ACN; Flow rate: 60 mL/min; Gradient: 10% B to 35% B in 9 min, 35% B; Wave Length: 220 nm; RT1(min): 7.85, 8.65(min)); Lyophilization yielded (3R,5R)-5-(3-(imidazo[1,2-c] pyrimidin-5-ylamino)-1H-pyrazol-5-yl) tetrahydrofuran-3-yl (1-methylcyclopropyl) carbamate (4.3 mg, 11 μmol, 9.9%) as a white solid. m/z (ES⁺) [M+H]⁺=384.15; HPLC tR=0.741 min. ¹H NMR (400 MHz, DMSO-d₆) δ 12.51 (s, 1H), 10.16 (s, 1H), 8.37 (s, 1H), 7.65 (s, 1H), 7.57 (s, 1H), 7.52 (s, 1H), 6.98 (s, 1H), 6.70 (s, 1H), 5.18 (s, 1H), 4.87 (s, 1H), 3.86 (s, 2H), 2.72 (s, 1H), 1.97 (s, 1H), 1.25 (s, 3H), 0.61 (s, 2H), 0.48 (s, 2H).

### Example 34

### (3R,5R)-5-(3-((2-(difluoromethyl)pyrazolo[1,5-a]pyrazin-4-yl)amino)-1H-pyrazol-5-yl)tetrahydrofuran-3-yl (1-methylcyclopropyl)carbamate

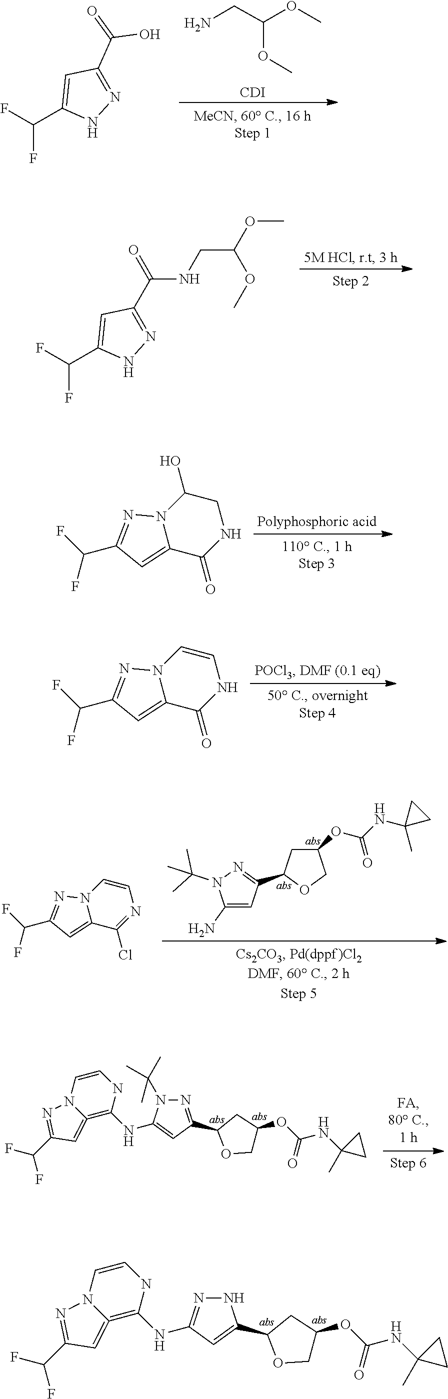

### 5-(difluoromethyl)-N-(2,2-dimethoxyethyl)-1H-pyrazole-3-carboxamide

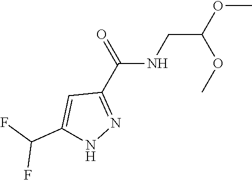 [SMILES: COC(CNC(=O)c1cc(C(F)F)[nH]n1)OC]

Step 1. To a mixture of 5-(difluoromethyl)-1H-pyrazole-3-carboxylic acid (2 g, 0.01 mol) and CDI (3 g, 0.02 mol) in MeCN (20 mL) was added 2,2-dimethoxyethan-1-amine (4 g, 0.04 mol) dropwise for 1.5 hour at 60° C. The mixture was stirred for 16 hours at 60° C. The reaction mixture was concentrated in vacuum. The crude product was purified by silica gel chromatography (6 g column; eluting with PE/EA; ratio:1/1). Concentration in vacuum resulted in 5-(difluoromethyl)-N-(2,2-dimethoxyethyl)-1H-pyrazole-3-carboxamide (5 g, 0.02 mol)(crude) as a tan sticky gum. m/z (ES⁺) [M+H]⁺=250.00; HPLC tR=0.608 min.

### 2-(difluoromethyl)-7-hydroxy-6,7-dihydropyrazolo[1,5-a]pyrazin-4(5H)-one

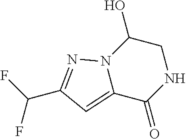 [SMILES: O=C1NCC(O)n2nc(C(F)F)cc21]

Step 2. A round bottomed flask was charged with 5-(difluoromethyl)-N-(2,2-dimethoxyethyl)-1H-pyrazole-3-carboxamide (5 g, 0.02 mol), HCl (5 M, 50 mL) and a stirbar. The solution was stirred for 3 hour at 25° C. The precipitated solids were collected by filtration and washed with EA (3*10 mL) to afford of 2-(difluoromethyl)-7-hydroxy-6,7-dihydropyrazolo[1,5-a]pyrazin-4(5H)-one (2.4 g, 12 mmol, 60%) as an off-white amorphous solid. m/z (ES⁺) [M+H]⁺=204.15 HPLC tR=0.173 min.

### 2-(difluoromethyl)pyrazolo[1,5-a]pyrazin-4(5H)-one

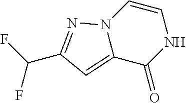 [SMILES: O=c1[nH]ccn2nc(C(F)F)cc12]

Step 3. A round bottomed flask was charged with 2-(difluoromethyl)-7-hydroxy-6,7-dihydropyrazolo[1,5-a]pyrazin-4(5H)-one (2.2 g, 1 Eq, 11 mmol), Polyphosphoric acid (20 mL) and a stirbar. The solution was stirred for 1 hour at 110° C. The reaction mixture was diluted with water (20 mL), and the aqueous phase was extracted with EA (30 mL) three times. The combined organic layers were washed with brine, dried over sodium sulfate, filtered, and concentrated in vacuum. The residue was purified by reverse flash chromatography with the following conditions: column, C18 silica gel; mobile phase, MeCN in water, 10% to 55% gradient in 10 min; detector, UV 254 nm to afford 2-(difluoromethyl)pyrazolo[1,5-a]pyrazin-4(5H)-one (1 g, 5 mmol, 50%) as an off-white amorphous solid. m/z (ES⁺) [M+H]⁺=186.00 HPLC tR=0.600 min.

### 4-chloro-2-(difluoromethyl)pyrazolo[1,5-a]pyrazine

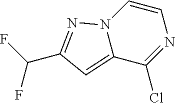 [SMILES: FC(F)c1cc2c(Cl)nccn2n1]

Step 4. A round bottomed flask was charged with 2-(difluoromethyl)pyrazolo[1,5-a]pyrazin-4(5H)-one (1 g, 1 Eq, 5 mmol), POCl₃ (10 mL), DMF (0.04 g, 0.1 Eq, 0.5 mmol) and a stirbar. The solution was stirred for 16 hour at 50° C. The reaction mixture was concentrated in vacuum. The reaction was poured into ice water and adjusted PH=7˜8 with Sat·NaHCO₃. and the aqueous phase was extracted with EA (30 mL) three times. The combined organic layers were washed with brine, dried over sodium sulfate, filtered, and concentrated in vacuum. The residue was purified by reverse flash chromatography with the following conditions: column, C18 silica gel; mobile phase, MeCN in water, 10% to 60% gradient in 10 min; detector, UV 254 nm to get 4-chloro-2-(difluoromethyl)pyrazolo[1,5-a]pyrazine (540 mg, 2.65 mmol, 50%) as an off-white amorphous solid. m/z (ES⁺) [M+H]=204.15; HPLC tR=0.603 min.

### (3R,5R)-5-(1-(tert-butyl)-5-((2-(difluoromethyl)pyrazolo[1,5-a]pyrazin-4-yl)amino)-1H-pyrazol-3-yl)tetrahydrofuran-3-yl (1-methylcyclopropyl)carbamate

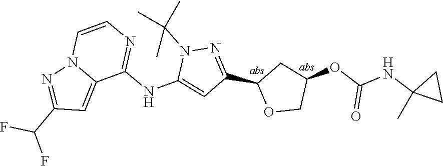 [SMILES: CC1(NC(=O)O[C@H]2CO[C@@H](c3cc(Nc4nccn5nc(C(F)F)cc45)n(C(C)(C)C)n3)C2)CC1]

Step 5. A round bottomed flask was charged with 4-chloro-2-(difluoromethyl)pyrazolo[1,5-a]pyrazine (95 mg, 0.47 mmol), DMF (2.5 mL), (3R,5R)-5-(5-amino-1-(tert-butyl)-1H-pyrazol-3-yl)tetrahydrofuran-3-yl (1-methylcyclopropyl)carbamate (0.15 g, 0.47 mmol), Cs₂CO₃ (0.45 g, 1.4 mmol), Pd(dppf)Cl2 (38 mg, 47 μmol), and a stirbar before being evacuated and purged with nitrogen three times. The mixture was stirred for 2 hour at 60° C. The reaction mixture was diluted with water (5 mL), and the aqueous phase was extracted with EA (15 mL) three times. The combined organic layers were washed with brine, dried over sodium sulfate, filtered, and concentrated in vacuum. The residue was purified by reverse flash chromatography with the following conditions: column, C18 silica gel; mobile phase, MeCN in water, 10% to 60% gradient in 10 min; detector, UV 254 nm to afford (3R,5R)-5-(1-(tert-butyl)-5-((2-(difluoromethyl)pyrazolo[1,5-a]pyrazin-4-yl)amino)-1H-pyrazol-3-yl)tetrahydrofuran-3-yl (1-methylcyclopropyl)carbamate (87 mg, 0.18 mmol, 38%) as yellow oil. m/z (ES⁺) [M+H]⁺=490.10 HPLC tR=0.808 min.

### (3R,5R)-5-(3-((2-(difluoromethyl)pyrazolo[1,5-a]pyrazin-4-yl)amino)-1H-pyrazol-5-yl)tetrahydrofuran-3-yl (1-methylcyclopropyl)carbamate

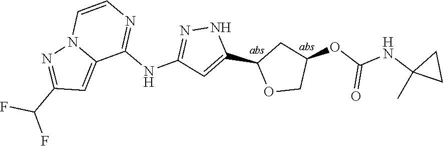 [SMILES: CC1(NC(=O)O[C@H]2CO[C@@H](c3cc(Nc4nccn5nc(C(F)F)cc45)n[nH]3)C2)CC1]

Step 6. A round bottomed flask was charged with (3R,5R)-5-(1-(tert-butyl)-5-((2-(difluoromethyl)pyrazolo[1,5-a]pyrazin-4-yl)amino)-1H-pyrazol-3-yl)tetrahydrofuran-3-yl (1-methylcyclopropyl)carbamate (82 mg, 0.17 mmol), FA (2 mL) and a stirbar. The solution was stirred for 1 hour at 80° C. and concentrated. The resulting crude material was purified by Pre-HPLC (Column: XBridge Prep OBD C18 Column, 30*150 mm, 5 m; Mobile Phase A: Water(10 mmol/L NH₄HCO₃), Mobile Phase B: ACN; Flow rate: 60 mL/min; Gradient: 23% B to 46% B in 8 min, 46% B; Wave Length: 254 nm; RT1(min): 7.23). Lyophilization yielded (3R,5R)-5-(3-((2-(difluoromethyl)pyrazolo[1,5-a]pyrazin-4-yl)amino)-1H-pyrazol-5-yl)tetrahydrofuran-3-yl (1-methylcyclopropyl)carbamate (41 mg, 95 μmol, 56%) as an off-white solid. m/z (ES⁺) [M+H]⁺=434.10; HPLC tR=0.721 min. ¹H NMR (400 MHz, DMSO-d₆) 12.43 (s, 1H), 10.27 (s, 1H), 8.16 (s, 1H), 7.53 (s, 3H), 7.20 (d, J=54.5 Hz, 1H), 6.81 (s, 1H), 5.17 (s, 1H), 4.86 (s, 1H), 3.86 (s, 2H), 2.72 (s, 1H), 1.95 (s, 1H), 1.25 (s, 3H), 0.61 (s, 2H), 0.48 (q, J=4.9, 4.4 Hz, 2H).

### Example 34

Additional compounds set forth in Table 12 were synthesized according to the procedures described herein.

TABLE 12
Additional Exemplary Compounds

| | | | | [α]²⁵_{D} |
|---|---|---|---|---|
| | | | MS | (C = |
| | | | [M + | 0.1, |
| Compound | Structure | Proton NMR | 1] | MeOH |
| N-(3-((1s,4s)-4-((4- isopropylpyridazin-3- yl)oxy)cyclohexyl)- 1H-pyrazol-5- yl)thiazolo[5,4- c]pyridin-4-amine | 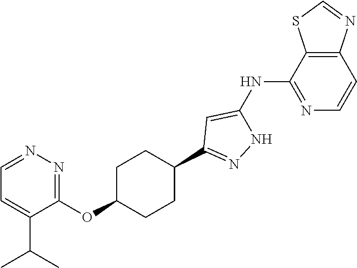 [SMILES: CC(C)c1ccnnc1O[C@H]1CC[C@@H](c2cc(Nc3nccc4ncsc34)[nH]n2)CC1] | ¹H NMR (400 MHz, DMSO-d₆) δ 12.15 (s, 1H), 9.43 (d, J = 19.4 Hz, 2H), 8.76 (d, J = 4.7 Hz, 1H), 8.12 (d, J = 5.6 Hz, 1H), 7.42 (dd, J = 19.2, 5.2 Hz, 2H), 6.39 (s, 1H), 5.55 (s, 1H), 3.20-3.09 (m, 1H), 2.83 (s, 1H), 2.12 (s, 2H), 1.90-1.80 (m, 6H), 1.24 (d, J = 6.9 Hz, 6H). | 436.15 | |
| rel-N-(5-((1R,3S)-3- ((4- isopropylpyridazin-3- yl)oxy)cyclopentyl)- 1H-pyrazol-3-yl)-3- (methoxymethyl)-1- methyl-1H-pyrazole- 5-carboxamide | 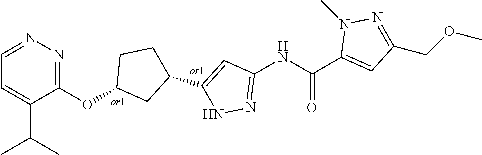 [SMILES: COCc1cc(C(=O)Nc2cc([C@H]3CC[C@@H](Oc4nnccc4C(C)C)C3)[nH]n2)n(C)n1] | ¹H NMR (400 MHz, DMSO-d₆) 12.26 (s, 1H), 10.73 (s, 1H), 8.76 (d, J = 4.7 Hz, 1H), 7.42 (dd, J = 4.8, 0.8 Hz, 1H), 7.12 (s, 1H), 6.46 (s, 1H), 5.71-5.65 (m, 1H), 4.34 (s, 2H), 4.05 (s, 3H), 3.27 (s, 3H), 3.22 (t, J = 7.8 Hz, 1H), 3.05 (p, J = 6.8 Hz, 1H), 2.68 (dt, J = 15.3, 7.7 Hz, 1H), 2.17-2.05 (m, 2H), 1.98 (s, 1H), 1.89 (ddd, J = 12.5, 8.3, 4.0 Hz, 2H), 1.17 (dd, J = 6.9, 1.1 Hz, 6H). | 440.30 | |
| rel-N-(5-((1R,3S)-3- ((4- isopropylpyridazin-3- yl)oxy)cyclopentyl)- 1H-pyrazol-3-yl)-3- (methoxymethyl)-1- methyl-1H-pyrazole- 5-carboxamide | 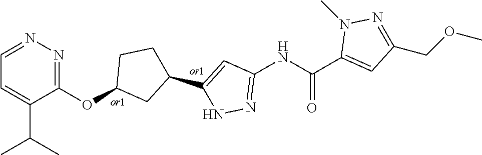 [SMILES: COCc1cc(C(=O)Nc2cc([C@@H]3CC[C@H](Oc4nnccc4C(C)C)C3)[nH]n2)n(C)n1] | ¹H NMR (400 MHz, DMSO-d₆) 12.26 (s, 1H), 10.73 (s, 1H), 8.76 (d, J = 4.7 Hz, 1H), 7.42 (dd, J = 4.7, 0.8 Hz, 1H), 7.12 (s, 1H), 6.47 (s, 1H), 5.71-5.65 (m, 1H), 4.34 (s, 2H), 4.05 (s, 3H), 3.27 (s, 3H), 3.20 (s, 1H), 3.05 (p, J = 6.9 Hz, 1H), 2.68 (dt, J = 14.8, 7.9 Hz, 1H), 2.18- 2.05 (m, 2H), 1.96 (d, J = 13.1 Hz, 1H), 1.89 (td, J = 12.4, 10.5, 6.2 Hz, 2H), 1.17 (dd, J = 6.9, 1.1 Hz, 6H). | 440.30 | |
| rel-N-(5-((1R,3S)-3- ((4- isopropylpyridazin-3- yl)oxy)cyclopentyl)- 1H-pyrazol-3- yl)thiazolo[5,4- c]pyridin-4-amine | 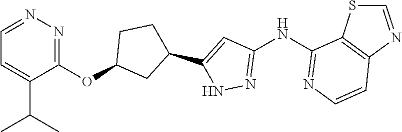 [SMILES: CC(C)c1ccnnc1O[C@H]1CC[C@@H](c2cc(Nc3nccc4ncsc34)n[nH]2)C1] | ¹H NMR (400 MHz, DMSO-d₆) 12.15 (s, 1H), 9.45 (s, 2H), 8.76 (d, J = 4.7 Hz, 2H), 8.11 (s, 1H), 7.41 (dd, J = 4.8, 0.8 Hz, 3H), 6.41 (s, 1H), 5.68 (s, 1H), 3.02 (p, J = 7.0 Hz, 2H), 2.14 (s, 2H), 1.97 (d, J = 12.9 Hz, 2H), 1.92 (d, J = 9.8 Hz, 2H), 1.14 (t, J = 6.6 Hz, 9H). | 422.20 | |
| rel-N-(5-((1R,3S)-3- ((4- isopropylpyridazin-3- yl)oxy)cyclopentyl)- 1H-pyrazol-3- yl)thiazolo[5,4- c]pyridin-4-amine | 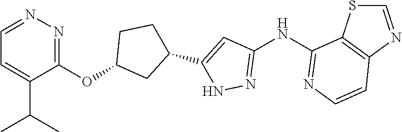 [SMILES: CC(C)c1ccnnc1O[C@@H]1CC[C@H](c2cc(Nc3nccc4ncsc34)n[nH]2)C1] | ¹H NMR (400 MHz, DMSO-d₆) 12.15 (s, 1H), 9.45 (s, 2H), 8.76 (d, J = 4.7 Hz, 1H), 8.11 (s, 1H), 7.41 (dd, J = 4.8, 0.8 Hz, 2H), 6.41 (s, 1H), 5.68 (s, 1H), 3.02 (p, J = 6.9 Hz, 2H), 2.13 (s, 1H), 1.99 (s, 1H), 1.96 (s, 1H), 1.93-1.87 (m, 2H), 1.14 (t, J = 6.6 Hz, 7H). | 422.20 | |
| rel-(3S,5S)-5-(3- (thiazolo[5,4- c]pyridin-4-ylamino)- 1H-pyrazol-5- yl)tetrahydrofuran-3- yl isopropylcarbamate | 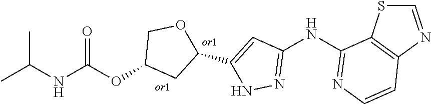 [SMILES: CC(C)NC(=O)O[C@@H]1CO[C@H](c2cc(Nc3nccc4ncsc34)n[nH]2)C1] | ¹H NMR (400 MHz, DMSO-d₆) 12.45 (d, J = 88.5 Hz, 1H), 9.50 (s, 2H), 8.17 (s, 1H), 7.44 (s, 1H), 7.20-7.04 (m, 1H), 6.59 (s, 1H), 5.18 (s, 1H), 4.84 (s, 1H), 3.86 (s, 2H), 3.59 (q, J = 6.8 Hz, 1H), 2.74 (d, J = 40.6 Hz, 1H), 2.38-1.88 (m, 1H), 1.15-0.78 (m, 6H). | 387.15 | |
| rel-(3R,5R)-5-(3- (thiazolo[5,4- c]pyridin-4-ylamino)- 1H-pyrazol-5- yl)tetrahydrofuran-3- yl isopropylcarbamate | 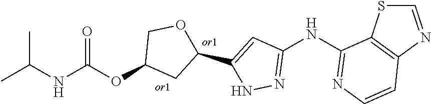 [SMILES: CC(C)NC(=O)O[C@H]1CO[C@@H](c2cc(Nc3nccc4ncsc34)n[nH]2)C1] | ¹H NMR (400 MHz, DMSO-d₆) 12.45 (d, J = 88.5 Hz, 1H), 9.50 (s, 2H), 8.17 (s, 1H), 7.44 (s, 1H), 7.20-7.04 (m, 1H), 6.59 (s, 1H), 5.18 (s, 1H), 4.84 (s, 1H), 3.86 (s, 2H), 3.59 (q, J = 6.8 Hz, 1H), 2.74 (d, J = 40.6 Hz, 1H), 2.38-1.88 (m, 1H), 1.15-0.78 (m, 6H). | 387.15 | |
| rel-(1R,3S)-3-(5- (thiazolo[4,5- d]pyrimidin-7- ylamino)-1H-pyrazol- 3-yl)cyclopentyl isopropylcarbamate | 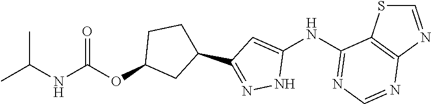 [SMILES: CC(C)NC(=O)O[C@H]1CC[C@@H](c2cc(Nc3ncnc4ncsc34)[nH]n2)C1] | ¹H NMR (400 MHz, DMSO-d₆) 12.39 (s, 1H), 10.34 (s, 1H), 9.62 (s, 1H), 8.63 (s, 1H), 6.96 (d, J = 7.7 Hz, 1H), 6.42 (s, 1H), 5.02 (s, 1H), 3.68-3.51 (m, 1H), 3.13 (d, J = 9.4 Hz, 1H), 2.53 (d, J = 8.5 Hz, 1H), 2.07 (q, J = 9.1 Hz, 1H), 2.00-1.86 (m, 1H), 1.75 (d, J = 8.6 Hz, 2H), 1.65 (s, 1H), 1.02 (t, J = 6.7 Hz, 6H). | 388.10 | |
| rel-(1R,3S)-3-(5- (thiazolo[4,5- d]pyrimidin-7- ylamino)-1H-pyrazol- 3- yl)cyclopentyliso- propylcarbamate | 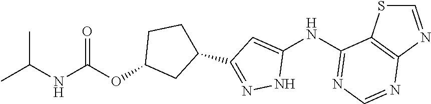 [SMILES: CC(C)NC(=O)O[C@@H]1CC[C@H](c2cc(Nc3ncnc4ncsc34)[nH]n2)C1] | ¹H NMR (400 MHz, DMSO-d₆) 12.39 (s, 1H), 10.34 (s, 1H), 9.62 (s, 1H), 8.63 (s, 1H), 6.96 (d, J = 7.7 Hz, 1H), 6.42 (s, 1H), 5.02 (s, 1H), 3.68-3.51 (m, 1H), 3.13 (d, J = 9.4 Hz, 1H), 2.53 (d, J = 8.5 Hz, 1H), 2.07 (q, J = 9.1 Hz, 1H), 2.00-1.86 (m, 1H), 1.75 (d, J = 8.6 Hz, 2H), 1.65 (s, 1H), 1.02 (t, J = 6.7 Hz, 6H). | 388.10 | |
| rel-(1R,3S)-3-(5-((2- acetamidothiazolo[5,4- c]pyridin-4- yl)amino)-1H- pyrazol-3- yl)cyclopentyl isopropylcarbamate | 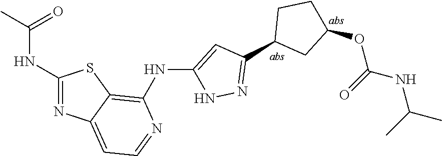 [SMILES: CC(=O)Nc1nc2ccnc(Nc3cc([C@H]4CC[C@@H](OC(=O)NC(C)C)C4)n[nH]3)c2s1] | ¹H NMR (400 MHz, DMSO-d₆) 12.46 (s, 1H), 12.06 (s, 1H), 9.18 (s, 1H), 8.06 (d, J = 5.7 Hz, 1H), 7.10 (d, J = 5.6 Hz, 1H), 6.96 (d, J = 7.8 Hz, 1H), 6.42 (d, J = 141.7 Hz, 1H), 5.01 (s, 1H), 3.68-3.50 (m, 1H), 3.07 (s, 1H), 2.47 (s, 1H), 2.22 (s, 3H), 2.13-1.97 (m, 1H), 1.90 (dt, J = 13.1, 6.5 Hz, 1H), 1.83-1.59 (m, 3H), 1.03 (dd, J = 6.7, 3.3 Hz, 6H). | 444.05 | |
| rel-(1R,3S)-3-(5-((2- acetamidothiazolo[5,4- c]pyridin-4- yl)amino)-1H- pyrazol-3- yl)cyclopentyl isopropylcarbamate | 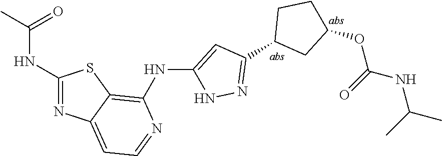 [SMILES: CC(=O)Nc1nc2ccnc(Nc3cc([C@@H]4CC[C@H](OC(=O)NC(C)C)C4)n[nH]3)c2s1] | ¹H NMR (400 MHz, DMSO-d₆) 12.46 (s, 1H), 12.06 (s, 1H), 9.18 (s, 1H), 8.06 (d, J = 5.7 Hz, 1H), 7.10 (d, J = 5.6 Hz, 1H), 6.96 (d, J = 7.8 Hz, 1H), 6.42 (d, J = 141.7 Hz, 1H), 5.01 (s, 1H), 3.68-3.50 (m, 1H), 3.07 (s, 1H), 2.47 (s, 1H), 2.22 (s, 3H), 2.13-1.97 (m, 1H), 1.90 (dt, J = 13.1, 6.5 Hz, 1H), 1.83-1.59 (m, 3H), 1.03 (dd, J = 6.7, 3.3 Hz, 6H). | 444.10 | |
| rel-N-(3-((1R,3S)-3- ((4- isopropylpyridazin-3- yl)oxy)cyclopentyl)- 1H-pyrazol-5-yl)-2- (methoxymethyl) thiazolo[5,4-c] pyridin-4-amine | 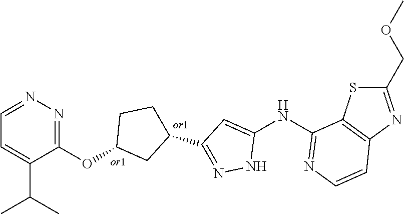 [SMILES: COCc1nc2ccnc(Nc3cc([C@H]4CC[C@@H](Oc5nnccc5C(C)C)C4)n[nH]3)c2s1] | ¹H NMR (400 MHz, DMSO-d₆) 12.14 (s, 1H), 9.34 (s, 1H), 8.76 (d, J = 4.7 Hz, 1H), 8.09 (d, J = 5.3 Hz, 1H), 7.41 (dd, J = 4.8, 0.8 Hz, 1H), 7.29 (s, 1H), 6.37 (s, 1H), 5.67 (t, J = 4.0 Hz, 1H), 4.84 (s, 2H), 3.45 (s, 3H), 3.24 (s, 1H), 3.02 (p, J = 6.8 Hz, 1H), 2.70 (s, 1H), 2.14 (s, 2H), 2.01-1.86 (m, 3H), 1.13 (t, J = 6.4 Hz, 6H). | 466.35 | |
| rel-N-(3-((1R,3S)-3- ((4- isopropylpyridazin-3- yl)oxy)cyclopentyl)- 1H-pyrazol-5-yl)-2- (methoxymethyl) thiazolo[5,4-c] pyridin-4-amine | 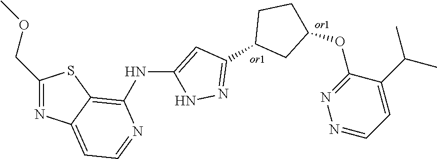 [SMILES: COCc1nc2ccnc(Nc3cc([C@@H]4CC[C@H](Oc5nnccc5C(C)C)C4)n[nH]3)c2s1] | ¹H NMR (400 MHz, DMSO-d₆) 12.17 (s, 1H), 9.40 (s, 1H), 8.76 (d, J = 4.7 Hz, 1H), 8.10 (d, J = 5.6 Hz, 1H), 7.41 (dd, J = 4.8, 0.8 Hz, 1H), 7.29 (d, J = 5.7 Hz, 1H), 6.32 (s, 1H), 5.70-5.63 (m, 1H), 4.84 (s, 2H), 3.45 (s, 3H), 3.30-3.17 (m, 1H), 3.02 (hept, J = 6.8 Hz, 1H), 2.76-2.64 (m, 1H), 2.12 (ddd, J = 14.9, 9.5, 6.7 Hz, 2H), 2.04-1.83 (m, 3H), 1.13 (dd, J = 6.9, 5.8 Hz, 6H). | 466.20 | |
| rel-N-(5-((1R,3S)-3- ((4- isopropylpyridazin-3- yl)oxy)cyclopentyl)- 1H-pyrazol-3- yl)acetamide | 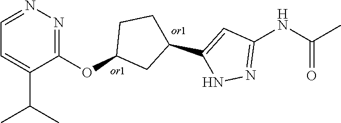 [SMILES: CC(=O)Nc1cc([C@@H]2CC[C@H](Oc3nnccc3C(C)C)C2)[nH]n1] | ¹H NMR (400 MHz, DMSO-d₆) 12.03 (s, 1H), 10.21 (s, 1H), 8.75 (d, J = 4.7 Hz, 1H), 7.42 (dd, J = 4.8, 0.8 Hz, 1H), 6.31 (s, 1H), 5.65 (s, 1H), 3.17 (q, J = 8.4 Hz, 1H), 3.02 (p, J = 6.9 Hz, 1H), 2.64 (d, J = 7.2 Hz, 1H), 2.08 (dt, J = 17.0, 6.8 Hz, 2H), 1.96 (s, 4H), 1.82 (q, J = 7.4, 5.7 Hz, 2H), 1.23-1.08 (m, 6H). | 330.15 | |
| rel-N-(5-((1R,3S)-3- ((4- isopropylpyridazin-3- yl)oxy)cyclopentyl)- 1H-pyrazol-3- yl)acetamide | 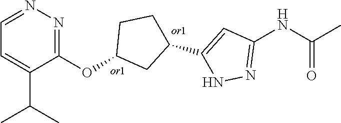 [SMILES: CC(=O)Nc1cc([C@H]2CC[C@@H](Oc3nnccc3C(C)C)C2)[nH]n1] | ¹H NMR (400 MHz, DMSO-d₆) 12.03 (s, 1H), 10.21 (s, 1H), 8.75 (d, J = 4.7 Hz, 1H), 7.42 (d, J = 4.8 Hz, 1H), 6.31 (s, 1H), 5.65 (d, J = 3.8 Hz, 1H), 3.26-3.10 (m, 1H), 3.02 (p, J = 6.9 Hz, 1H), 2.65 (s, 1H), 2.19-2.04 (m, 2H), 1.96 (s, 4H), 1.83 (t, J = 8.9 Hz, 2H), 1.16 (d, J = 6.8 Hz, 6H). | 330.15 | |
| N-(5-((1s,3s)-3-((4- isopropylpyridazin-3- yl)oxy)cyclobutyl)- 1H-pyrazol-3- yl)thiazolo[5,4- c]pyridin-4-amine | 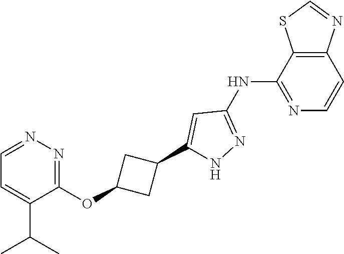 [SMILES: CC(C)c1ccnnc1O[C@H]1C[C@@H](c2cc(Nc3nccc4ncsc34)n[nH]2)C1] | ¹H NMR (400 MHz, DMSO-d₆) 12.24 (s, 1H), 9.48 (s, 2H), 8.79 (d, J = 4.7 Hz, 1H), 8.14 (d, J = 5.5 Hz, 1H), 7.57-7.13 (m, 2H), 6.49 (s, 1H), 5.36 (p, J = 7.4 Hz, 1H), 3.29-3.20 (m, 1H), 3.15-3.02 (m, 1H), 3.01-2.81 (m, 2H), 2.36-2.07 (m, 2H), 1.22 (d, J = 6.9 Hz, 6H). | 408.10 | |
| rel-N-(5-((1R,3S)-3- ((2-methylpyridin-3- yl)oxy)cyclopentyl)- 1H-pyrazol-3- yl)pyrido[2,3- d]pyrimidin-4-amine | 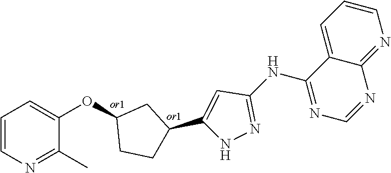 [SMILES: Cc1ncccc1O[C@@H]1CC[C@H](c2cc(Nc3ncnc4ncccc34)n[nH]2)C1] | ¹H NMR (400 MHz, DMSO-d₆) 12.35 (s, 1H), 10.67 (s, 1H), 9.11-9.01 (m, 2H), 8.72 (s, 1H), 7.99 (dd, J = 4.7, 1.3 Hz, 1H), 7.59 (dd, J = 8.3, 4.4 Hz, 1H), 7.33 (dd, J = 8.2, 1.4 Hz, 1H), 7.18 (dd, J = 8.3, 4.8 Hz, 1H), 6.72 (s, 1H), 4.97 (s, 1H), 3.30-3.21 (m, 1H), 2.64 (dd, J = 14.8, 7.5 Hz, 1H), 2.36 (s, 3H), 2.15 (q, J = 8.8, 8.1 Hz, 1H), 2.06 (s, 1H), 2.04-1.81 (m, 3H). | 388.10 | |
| rel-N-(5-((1R,3S)-3- ((2-methylpyridin-3- yl)oxy)cyclopentyl)- 1H-pyrazol-3- yl)pyrido[2,3- d]pyrimidin-4-amine | 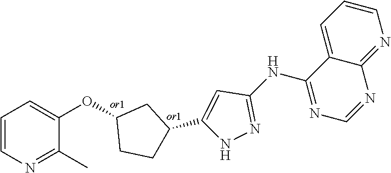 [SMILES: Cc1ncccc1O[C@H]1CC[C@@H](c2cc(Nc3ncnc4ncccc34)n[nH]2)C1] | ¹H NMR (400 MHz, DMSO-d₆) 12.35 (s, 1H), 10.68 (s, 1H), 9.10-9.01 (m, 2H), 8.72 (s, 1H), 7.99 (dd, J = 4.8, 1.3 Hz, 1H), 7.59 (dd, J = 7.9, 4.3 Hz, 1H), 7.33 (dd, J = 8.3, 1.4 Hz, 1H), 7.18 (dd, J = 8.2, 4.7 Hz, 1H), 6.72 (s, 1H), 4.97 (d, J = 6.4 Hz, 1H), 3.30-3.21 (m, 1H), 2.64 (dt, J = 14.5, 6.9 Hz, 1H), 2.36 (s, 3H), 2.19-2.03 (m, 2H), 1.99-1.81 (m, 3H). | 388.20 | |
| rel-N-(5-((1R,3S)-3- ((2-methylpyridin-3- yl)oxy)cyclopentyl)- 1H-pyrazol-3- yl)pyrazolo[1,5- a]pyrazin-4-amine | 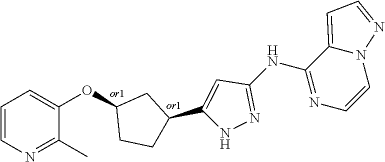 [SMILES: Cc1ncccc1O[C@@H]1CC[C@H](c2cc(Nc3nccn4nccc34)n[nH]2)C1] | ¹H NMR (400 MHz, DMSO-d₆) 12.14 (s, 1H), 9.99 (s, 1H), 8.07 (d, J = 4.7 Hz, 1H), 8.02-7.93 (m, 2H), 7.42-7.27 (m, 3H), 7.17 (dd, J = 8.3, 4.7 Hz, 1H), 6.68 (s, 1H), 4.99-4.92 (m, 1H), 3.21 (q, J = 8.2 Hz, 1H), 2.62 (ddd, J = 14.5, 8.7, 6.3 Hz, 1H), 2.37 (s, 3H), 2.13 (d, J = 8.6 Hz, 1H), 2.07-1.97 (m, 1H), 1.97-1.79 (m, 3H). | 376.30 | −5.00 |
| rel-N-(5-((1R,3S)-3- ((2-methylpyridin-3- yl)oxy)cyclopentyl)- 1H-pyrazol-3- yl)pyrazolo[1,5- a]pyrazin-4-amine | 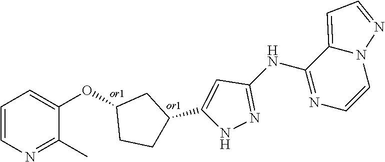 [SMILES: Cc1ncccc1O[C@H]1CC[C@@H](c2cc(Nc3nccn4nccc34)n[nH]2)C1] | ¹H NMR (400 MHz, DMSO-d₆) 12.14 (s, 1H), 9.99 (s, 1H), 8.07 (d, J = 4.8 Hz, 1H), 8.02-7.92 (m, 2H), 7.43-7.24 (m, 3H), 7.17 (dd, J = 8.2, 4.7 Hz, 1H), 6.68 (s, 1H), 4.96 (q, J = 6.0, 4.5 Hz, 1H), 3.28-3.12 (m, 1H), 2.70-2.56 (m, 1H), 2.37 (s, 3H), 2.11 (dd, J = 15.2, 6.9 Hz, 1H), 2.07-1.99 (m, 1H), 1.98-1.75 (m, 3H). | 376.30 | 5 |
| rel-N-(5-((1R,3S)-3- (pyridin-3- yloxy)cyclopentyl)- 1H-pyrazol-3- yl)pyrido[2,3- d]pyrimidin-4-amine | 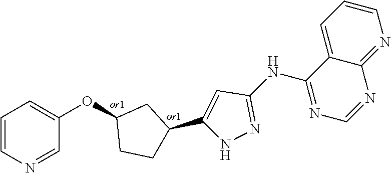 [SMILES: c1cncc(O[C@@H]2CC[C@H](c3cc(Nc4ncnc5ncccc45)n[nH]3)C2)c1] | ¹H NMR (400 MHz, DMSO-d₆) 12.36 (s, 1H), 10.69-10.65 (m, 1H), 9.05 (dd, J = 15.8, 6.2 Hz, 3H), 8.75 (s, 1H), 8.30 (d, J = 2.9 Hz, 2H), 8.16 (d, J = 4.5 Hz, 2H), 7.59 (dd, J = 8.3, 4.4 Hz, 2H), 7.44-7.37 (m, 2H), 7.33 (dd, J = 8.5, 4.5 Hz, 2H), 6.73 (s, 1H), 5.02 (d, J = 6.0 Hz, 2H), 3.22 (q, J = 8.6 Hz, 2H), 2.67 (p, J = 7.3 Hz, 2H), 2.12 (d, J = 6.9 Hz, 1H), 2.10-2.01 (m, 2H), 2.00-1.77 (m, 6H). | 374.10 | |
| rel-N-(5-((1R,3S)-3- (pyridin-3- yloxy)cyclopentyl)- 1H-pyrazol-3- yl)pyrido[2,3- d]pyrimidin-4-amine | 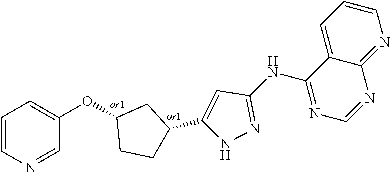 [SMILES: c1cncc(O[C@H]2CC[C@@H](c3cc(Nc4ncnc5ncccc45)n[nH]3)C2)c1] | ¹H NMR (400 MHz, DMSO-d₆) 12.36 (s, 1H), 10.67 (s, 1H), 9.06 (dd, J = 14.9, 6.2 Hz, 2H), 8.75 (s, 1H), 8.30 (d, J = 2.9 Hz, 1H), 8.16 (d, J = 4.6 Hz, 1H), 7.59 (dd, J = 8.4, 4.4 Hz, 1H), 7.44-7.37 (m, 1H), 7.33 (dd, J = 8.5, 4.5 Hz, 1H), 6.73 (s, 1H), 5.02 (d, J = 6.2 Hz, 1H), 3.27-3.19 (m, 1H), 2.67 (p, J = 7.4 Hz, 1H), 2.12 (d, J = 7.7 Hz, 1H), 2.07 (q, J = 6.2 Hz, 1H), 1.96-1.77 (m, 3H). | 374.10 | |
| N-(5-((1s,3s)-3-(((2- methylpyridin-3- yl)oxy)methyl)cyclo- butyl)-1H-pyrazol-3- yl)pyrazolo[1,5- a]pyrazin-4-amine | 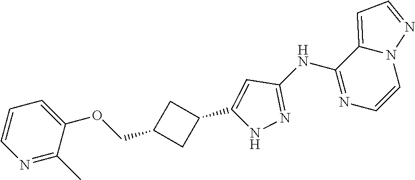 [SMILES: Cc1ncccc1OC[C@H]1C[C@@H](c2cc(Nc3nccn4nccc34)n[nH]2)C1] | ¹H NMR (400 MHz, DMSO-d₆) 12.14 (s, 1H), 10.02 (s, 1H), 8.08 (d, J = 4.8 Hz, 1H), 8.03-7.94 (m, 4-2H), 7.38 (d, J = 4.8 Hz, 3H), 7.18 (dd, J = 8.2, 4.7 Hz, 1H), 6.71 (s, 1H), 4.02 (d, J = 5.7 Hz, 2H), 3.46 (q, J = 9.0, 8.1 Hz, 1H), 2.90-2.70 (m, 1H), 2.41 (s, 5H), 2.12 (qd, J = 9.6, 2.6 Hz, 2H). | 376.15 | |
| N-(5-((1r,3r)-3-(((2- methylpyridin-3- yl)oxy)methyl)cyclo- butyl)-1H-pyrazol-3- yl)pyrazolo[1,5- a]pyrazin-4-amine | 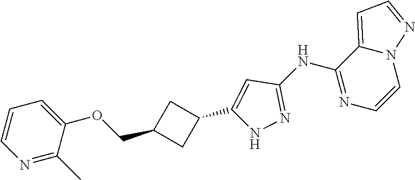 [SMILES: Cc1ncccc1OC[C@H]1C[C@H](c2cc(Nc3nccn4nccc34)n[nH]2)C1] | ¹H NMR (400 MHz, DMSO-d₆) 12.21 (s, 1H), 10.07 (s, 1H), 8.08 (d, J = 4.8 Hz, 1H), 8.03 (dd, J = 4.8, 1.3 Hz, 1H), 7.97 (d, J = 2.3 Hz, 1H), 7.44-7.38 (m, 2H), 7.35 (d, J = 2.4 Hz, 1H), 7.23 (dd, J = 8.2, 4.8 Hz, 1H), 6.69 (s, 1H), 4.16 (d, J = 6.6 Hz, 2H), 3.68 (p, J = 8.2 Hz, 1H), 2.88-2.76 (m, 1H), 2.41 (s, 3H), 2.40-2.27 (m, 4H). | 376.15 | |
| rel-2- (methoxymethyl)-N- (3-((1R,3S)-3-((2- methylpyridin-3- yl)oxy)cyclopentyl)- 1H-pyrazol-5- yl)pyrazolo[1,5- a]pyrazin-4-amine | 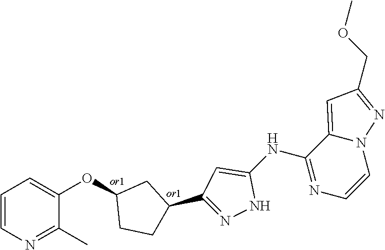 [SMILES: COCc1cc2c(Nc3cc([C@H]4CC[C@@H](Oc5cccnc5C)C4)n[nH]3)nccn2n1] | ¹H NMR (400 MHz, Chloroform-d) 8.11 (dd, J = 3.8, 2.4 Hz, 1H), 7.87 (d, J = 4.8 Hz, 1H), 7.40 (d, J = 4.8 Hz, 1H), 7.18-7.09 (m, 2H), 6.86 (s, 1H), 6.50 (s, 1H), 4.91 (s, 1H), 4.68 (s, 2H), 3.47 (s, 3H), 3.36 (q, J = 8.0 Hz, 1H), 3.01 (s, 1H), 2.63 (ddd, J = 14.9, 9.2, 6.2 Hz, 1H), 2.53 (s, 3H), 2.25 (d, J = 4.7 Hz, 1H), 2.14 (d, J = 5.9 Hz, 1H), 2.121.98 (m, 1H), 1.52-1.40 (m, 1H), 1.33 (s, 1H). | 420.15 | |
| rel-2- (methoxymethyl)-N- (3-((1R,3S)-3-((2- methylpyridin-3- yl)oxy)cyclopentyl)- 1H-pyrazol-5- yl)pyrazolo[1,5- a]pyrazin-4-amine | 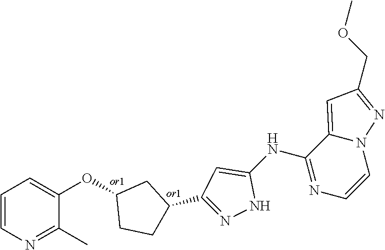 [SMILES: COCc1cc2c(Nc3cc([C@@H]4CC[C@H](Oc5cccnc5C)C4)n[nH]3)nccn2n1] | ¹H NMR (400 MHz, Chloroform-d) 8.10 (t, J = 3.1 Hz, 1H), 7.87 (s, 1H), 7.41 (s, 1H), 7.11 (d, J = 3.1 Hz, 2H), 6.80 (s, 1H), 6.50 (s, 1H), 4.90 (s, 1H), 4.68 (s, 2H), 3.47 (s, 3H), 3.36 (t, J = 8.1 Hz, 1H), 3.02 (s, 1H), 2.63 (ddd, J = 14.8, 9.1, 6.2 Hz, 2H), 2.52 (s, 3H), 2.24 (s, 1H), 2.14 (s, 1H), 2.10-2.01 (m, 1H). | 420.15 | |
| rel-N-(5-((1R,3S)-3- (pyridin-3- yloxy)cyclopentyl)- 1H-pyrazol-3- yl)pyrazolo[1,5- a]pyrazin-4-amine | 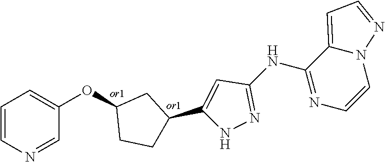 [SMILES: c1cncc(O[C@@H]2CC[C@H](c3cc(Nc4nccn5nccc45)n[nH]3)C2)c1] | ¹H NMR (400 MHz, DMSO-d₆) 12.14 (s, 1H), 9.98 (s, 1H), 8.30 (d, J = 2.9 Hz, 1H), 8.16 (dd, J = 4.5, 1.4 Hz, 1H), 8.07 (d, J = 4.7 Hz, 1H), 7.96 (d, J = 2.3 Hz, 1H), 7.42-7.37 (m, 2H), 7.33 (dd, J = 8.4, 4.5 Hz, 2H), 6.69 (s, 1H), 5.04-4.98 (m, 1H), 3.20 (p, J = 8.7 Hz, 1H), 2.66 (dt, J = 14.3, 7.3 Hz, 1H), 2.16-2.00 (m, 2H), 1.97-1.74 (m, 3H). | 362.15 | −28 |
| rel-N-(5-((1R,3S)-3- (pyridin-3- yloxy)cyclopentyl)- 1H-pyrazol-3- yl)pyrazolo[1,5- a]pyrazin-4-amine | 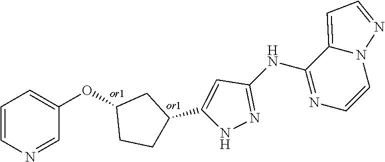 [SMILES: c1cncc(O[C@H]2CC[C@@H](c3cc(Nc4nccn5nccc45)n[nH]3)C2)c1] | ¹H NMR (400 MHz, DMSO-d₆) 12.15 (s, 1H), 10.00 (s, 1H), 8.30 (d, J = 2.9 Hz, 1H), 8.16 (dd, J = 4.5, 1.4 Hz, 1H), 8.07 (d, J = 4.8 Hz, 1H), 7.96 (d, J = 2.3 Hz, 1H), 7.44-7.36 (m, 2H), 7.33 (dd, J = 8.4, 4.6 Hz, 2H), 6.67 (s, 1H), 5.01 (dd, J = 6.8, 4.0 Hz, 1H), 3.26-3.13 (m, 1H), 2.66 (dt, J = 14.3, 7.6 Hz, 1H), 2.09 (ddt, J = 19.4, 16.2, 5.5 Hz, 2H), 1.99-1.76 (m, 3H). | 362.20 | 22 |
| rel-2- (methoxymethyl)-N- (3-((1R,3S)-3- (pyridin-3- yloxy)cyclopentyl)- 1H-pyrazol-5- yl)pyrazolo[1,5- a]pyrazin-4-amine | 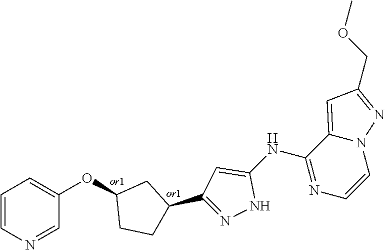 [SMILES: COCc1cc2c(Nc3cc([C@H]4CC[C@@H](Oc5cccnc5)C4)n[nH]3)nccn2n1] | ¹H NMR (400 MHz, DMSO-d₆) 12.15 (s, 1H), 9.98 (s, 1H), 8.30 (d, J = 2.8 Hz, 1H), 8.16 (dd, J = 4.5, 1.4 Hz, 1H), 8.02 (d, J = 4.8 Hz, 1H), 7.43-7.36 (m, 2H), 7.33 (dd, J = 8.4, 4.6 Hz, 1H), 7.28 (s, 1H), 6.66 (s, 1H), 5.01 (dd, J = 6.8, 3.8 Hz, 1H), 4.56 (s, 2H), 3.33 (s, 3H), 3.28-3.08 (m, 1H), 2.75-2.55 (m, 1H), 2.19-1.97 (m, 2H), 1.98-1.64 (m, 3H). | 406.30 | |
| rel-2- (methoxymethyl)-N- (3-((1R,3S)-3- (pyridin-3- yloxy)cyclopentyl)- 1H-pyrazol-5- yl)pyrazolo[1,5- a]pyrazin-4-amine | 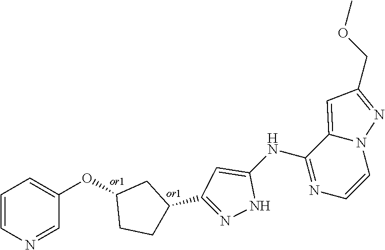 [SMILES: COCc1cc2c(Nc3cc([C@@H]4CC[C@H](Oc5cccnc5)C4)n[nH]3)nccn2n1] | ¹H NMR (400 MHz, DMSO-d₆) 12.15 (s, 1H), 9.98 (s, 1H), 8.30 (d, J = 2.9 Hz, 1H), 8.16 (dd, J = 4.5, 1.4 Hz, 1H), 8.02 (d, J = 4.8 Hz, 1H), 7.45-7.36 (m, 2H), 7.36-7.19 (m, 2H), 6.65 (s, 1H), 5.01 (dq, J = 6.7, 3.8, 3.0 Hz, 1H), 4.56 (s, 2H), 3.33 (s, 3H), 3.26-3.08 (m, 1H), 2.65 (ddd, J = 14.3, 7.9, 6.0 Hz, 1H), 2.18-1.98 (m, 2H), 2.00-1.67 (m, 3H). | 406.30 | |
| rel-N-(5-((2R,4R)-4- ((2-methylpyridin-3- yl)oxy)tetrahydrofuran- 2-yl)-1H-pyrazol-3- yl)pyrazolo[1,5- a]pyrazin-4-amine | 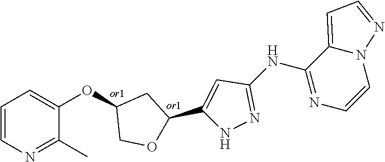 [SMILES: Cc1ncccc1O[C@@H]1CO[C@H](c2cc(Nc3nccn4nccc34)n[nH]2)C1] | ¹H NMR (400 MHz, DMSO-d₆) 12.41 (s, 1H), 10.06 (s, 1H), 8.09 (s, 1H), 8.01 (dd, J = 4.8, 1.3 Hz, 1H), 7.97 (s, 1H), 7.38-7.29 (m, 3H), 7.18 (dd, J = 8.3, 4.8 Hz, 1H), 6.82 (s, 1H), 5.17 (s, 1H), 5.06 (s, 1H), 4.06 (d, J = 11.5 Hz, 2H), 2.83 (dt, J = 14.3, 7.3 Hz, 1H), 2.34 (s, 3H), 2.17 (dd, J = 13.7, 5.8 Hz, 1H). | 378.10 | −8 |
| rel-N-(5-((2R,4R)-4- ((2-methylpyridin-3- yl)oxy)tetrahydrofuran- 2-yl)-1H-pyrazol-3- yl)pyrazolo[1,5- a]pyrazin-4-amine | 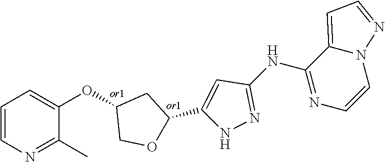 [SMILES: Cc1ncccc1O[C@H]1CO[C@@H](c2cc(Nc3nccn4nccc34)n[nH]2)C1] | ¹H NMR (400 MHz, DMSO-d₆) 12.41 (s, 1H), 10.07 (s, 1H), 8.09 (s, 1H), 8.04-7.90 (m, 2H), 7.43-7.25 (m, 3H), 7.18 (dd, J = 8.2, 4.7 Hz, 1H), 6.82 (s, 1H), 5.17 (s, 1H), 5.05 (s, 1H), 4.12-4.02 (m, 2H), 2.83 (dt, J = 14.2, 7.4 Hz, 1H), 2.34 (s, 3H), 2.22-2.12 (m, 1H). | 378.10 | 12 |
| N-(5-((1s,3s)-3- ((pyridin-3- yloxy)methyl)cyclo- butyl)-1H-pyrazol-3- yl)pyrazolo[1,5- a]pyrazin-4-amine | 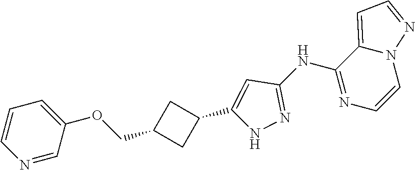 [SMILES: c1cncc(OC[C@H]2C[C@@H](c3cc(Nc4nccn5nccc45)n[nH]3)C2)c1] | ¹H NMR (400 MHz, DMSO-d₆) 12.18 (s, 1H), 10.03 (s, 1H), 8.34 (d, J = 2.9 Hz, 1H), 8.18 (dd, J = 4.6, 1.3 Hz, 1H), 8.08 (d, J = 4.8 Hz, 1H), 7.97 (d, J = 2.3 Hz, 1H), 7.48-7.38 (m, 2H), 7.38-7.31 (m, 2H), 6.70 (s, 1H), 4.20 (d, J = 7.0 Hz, 2H), 3.73-3.61 (m, 1H), 2.88-2.73 (m, 1H), 2.41-2.23 (m, 4H). | 362.25 | |
| N-(5-((1r,3r)-3- ((pyridin-3- yloxy)methyl)cyclo- butyl)-1H-pyrazol-3- yl)pyrazolo[1,5- a]pyrazin-4-amine | 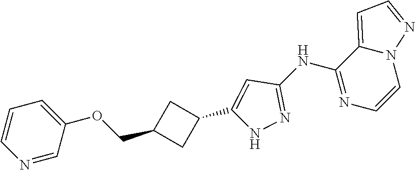 [SMILES: c1cncc(OC[C@H]2C[C@H](c3cc(Nc4nccn5nccc45)n[nH]3)C2)c1] | ¹H NMR (400 MHz, DMSO-d₆) 12.16 (s, 1H), 10.02 (s, 1H), 8.34 (d, J = 2.9 Hz, 1H), 8.17 (dd, J = 4.6, 1.4 Hz, 1H), 8.08 (d, J = 4.8 Hz, 1H), 7.96 (d, J = 2.3 Hz, 1H), 7.45-7.37 (m, 2H), 7.37-7.30 (m, 2H), 6.69 (s, 1H), 4.08 (d, J = 6.3 Hz, 2H), 3.52-3.39 (m, 1H), 2.81-2.68 (m, 1H), 2.51-2.43 (m, 2H), 2.06 (qd, J = 9.5, 2.6 Hz, 2H). | 362.20 | |
| rel-N-(5-((1R,3S)-3- (2-(isopropylamino)- 2-oxoethyl)-3- methylcyclopentyl)- 1H-pyrazol-3-yl)-3- (methoxymethyl)-1- methyl-1H-pyrazole- 5-carboxamide | 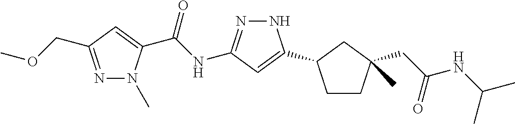 [SMILES: COCc1cc(C(=O)Nc2cc([C@H]3CC[C@@](C)(CC(=O)NC(C)C)C3)[nH]n2)n(C)n1] | ¹H NMR (400 MHz, DMSO-d₆) 12.18 (s, 1H), 10.72 (s, 1H), 7.65 (d, J = 7.7 Hz, 1H), 7.12 (s, 1H), 6.40 (s, 1H), 4.34 (s, 2H), 4.05 (s, 3H), 3.84 (dq, J = 13.5, 6.7 Hz, 1H), 3.27 (s, 3H), 3.18 (q, J = 8.9, 8.5 Hz, 1H), 2.10 (s, 3H), 1.83 (dt, J = 11.6, 7.6 Hz, 2H), 1.68 (ddd, J = 21.7, 18.9, 10.1 Hz, 2H), 1.48-1.30 (m, 1H), 1.08- 0.99 (m, 9H). | 417.40 | |
| rel-N-(5-((1R,3S)-3- (2-(isopropylamino)- 2-oxoethyl)-3- methylcyclopentyl)- 1H-pyrazol-3-yl)-3- (methoxymethyl)-1- methyl-1H-pyrazole- 5-carboxamide | 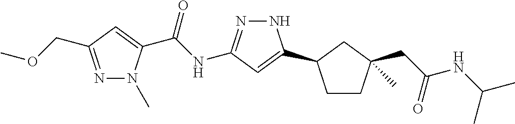 [SMILES: COCc1cc(C(=O)Nc2cc([C@@H]3CC[C@](C)(CC(=O)NC(C)C)C3)[nH]n2)n(C)n1] | ¹H NMR (400 MHz, DMSO-d₆) 12.17 (s, 1H), 10.71 (s, 1H), 7.64 (d, J = 7.7 Hz, 1H), 7.12 (s, 1H), 6.39 (s, 1H), 4.34 (s, 2H), 4.05 (s, 3H), 3.92-3.71 (m, 1H), 3.27 (s, 3H), 3.20 (p, J = 8.6 Hz, 1H), 2.15-2.00 (m, 3H), 1.90-1.76 (m, 2H), 1.76-1.60 (m, 2H), 1.42 (dt, J = 12.5, 7.6 Hz, 1H), 1.11-0.99 (m, 9H). | 417.40 | |
| rel-N-(5-((1R,3R)-3- (2-(isopropylamino)- 2-oxoethyl)-3- methylcyclopentyl)- 1H-pyrazol-3-yl)-3- (methoxymethyl)-1- methyl-1H-pyrazole- 5-carboxamide | 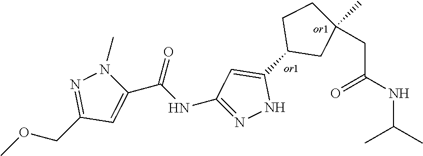 [SMILES: COCc1cc(C(=O)Nc2cc([C@@H]3CC[C@@](C)(CC(=O)NC(C)C)C3)[nH]n2)n(C)n1] | ¹H NMR (400 MHz, DMSO-d₆) 12.19 (s, 1H), 10.72 (s, 1H), 7.68 (d, J = 7.8 Hz, 1H), 7.12 (s, 1H), 6.38 (s, 1H), 4.34 (s, 2H), 4.05 (s, 3H), 3.95-3.76 (m, 1H), 3.27 (s, 3H), 3.23-3.05 (m, 1H), 2.21 (dd, J = 12.8, 8.2 Hz, 1H), 2.15-1.98 (m, 3H), 1.72 (dddd, J = 21.0, 16.7, 10.4, 7.9 Hz, 2H), 1.50 (ddd, J = 10.8, 7.2, 4.1 Hz, 1H), 1.40 (dd, J = 12.8, 10.0 Hz, 1H), 1.11 (s, 3H), 1.08-0.97 (m, 6H). | 417.40 | |
| rel-N-(5-((1R,3R)-3- (2-(isopropylamino)- 2-oxoethyl)-3- methylcyclopentyl)- 1H-pyrazol-3-yl)-3- (methoxymethyl)-1- methyl-1H-pyrazole- 5-carboxamide | 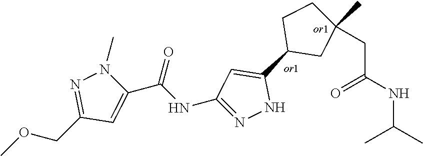 [SMILES: COCc1cc(C(=O)Nc2cc([C@H]3CC[C@](C)(CC(=O)NC(C)C)C3)[nH]n2)n(C)n1] | ¹H NMR (400 MHz, DMSO-d₆) 12.19 (s, 1H), 10.72 (s, 1H), 7.67 (d, J = 7.7 Hz, 1H), 7.12 (s, 1H), 6.38 (s, 1H), 4.34 (s, 2H), 4.05 (s, 3H), 3.92-3.68 (m, 1H), 3.27 (s, 3H), 3.18 (h, J = 8.4, 7.7 Hz, 1H), 2.21 (dd, J = 12.8, 8.2 Hz, 1H), 2.11-1.97 (m, 3H), 1.80-1.58 (m, 2H), 1.50 (ddd, J = 10.7, 7.2, 4.0 Hz, 1H), 1.40 (dd, J = 12.8, 10.0 Hz, 1H), 1.10 (s, 3H), 1.06-1.01 (m, 6H), . | 417.40 | |
| N-(5-((1r,3r)-3-(2- (isopropylamino)-2- oxoethyl)-3- methylcyclobutyl)- 1H-pyrazol-3-yl)-3- (methoxymethyl)-1- methyl-1H-pyrazole- 5-carboxamide | 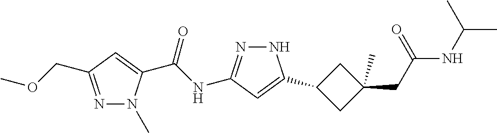 [SMILES: COCc1cc(C(=O)Nc2cc([C@H]3C[C@](C)(CC(=O)NC(C)C)C3)[nH]n2)n(C)n1] | ¹H NMR (400 MHz, DMSO-d₆) 12.21 (s, 1H), 10.72 (s, 1H), 7.73 (d, J = 7.7 Hz, 1H), 7.12 (s, 1H), 6.43 (s, 1H), 4.34 (s, 2H), 4.06 (s, 3H), 3.86 (dq, J = 13.5, 6.7 Hz, 1H), 3.44 (p, J = 8.9 Hz, 1H), 3.27 (s, 3H), 2.48-2.38 (m, 2H), 2.25 (s, 2H), 1.89 (dd, J = 12.1, 9.3 Hz, 2H), 1.15 (s, 3H), 1.06 (d, J = 6.6 Hz, 6H). | 403.20 | |
| N-(5-((1s,3s)-3-(2- (isopropylamino)-2- oxoethyl)-3- methylcyclobutyl)- 1H-pyrazol-3-yl)-3- (methoxymethyl)-1- methyl-1H-pyrazole- 5-carboxamide | 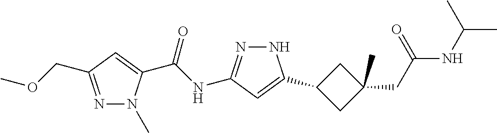 [SMILES: COCc1cc(C(=O)Nc2cc([C@H]3C[C@@](C)(CC(=O)NC(C)C)C3)[nH]n2)n(C)n1] | ¹H NMR (400 MHz, DMSO-d₆) 12.16 (s, 1H), 10.71 (s, 1H), 7.63 (d, J = 7.7 Hz, 1H), 7.12 (s, 1H), 6.46 (s, 1H), 4.34 (s, 2H), 4.06 (s, 3H), 3.84 (dp, J = 7.6, 6.5 Hz, 1H), 3.47 (p, J = 9.0 Hz, 1H), 3.27 (s, 3H), 2.22-2.11 (m, 4H), 2.05 (td, J = 8.9, 2.5 Hz, 2H), 1.24 (s, 3H), 1.04 (d, J = 6.6 Hz, 6H). | 403.20 | |
| rel-N-(5-((1R,3S)-3- ((4-cyclo- propylisothiazol- 3- yl)oxy)cyclopentyl)- 1H-pyrazol-3-yl)-3- (methoxymethyl)-1- methyl-1H-pyrazole- 5-carboxamide | 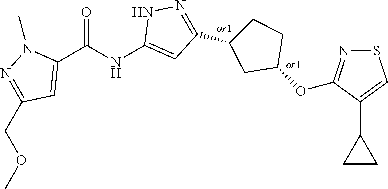 [SMILES: COCc1cc(C(=O)Nc2cc([C@@H]3CC[C@H](Oc4nscc4C4CC4)C3)n[nH]2)n(C)n1] | ¹H NMR (400 MHz, DMSO) 12.24 (s, 1H), 10.71 (s, 1H), 8.26 (s, 1H), 7.11 (s, 1H), 6.47 (s, 1H), 5.35 (s, 1H), 4.33 (s, 2H), 4.04 (s, 3H), 3.27 (s, 3H), 3.18 (d, J = 8.2 Hz, 1H), 2.62 (dt, J = 14.4, 7.5 Hz, 1H), 2.15-1.88 (m, 3H), 1.92- 1.70 (m, 3H), 0.86 (dt, J = 10.4, 3.0 Hz, 2H), 0.70-0.59 (m, 2H). | 443.15 | |
| rel-N-(5-((1R,3S)-3- ((4- cyclopropylisothiazol- 3- yl)oxy)cyclopentyl)- 1H-pyrazol-3-yl)-3- (methoxymethyl)-1- methyl-1H-pyrazole- 5-carboxamide | 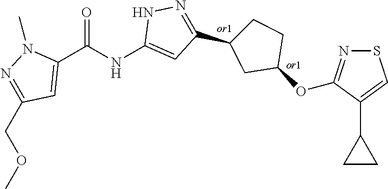 [SMILES: COCc1cc(C(=O)Nc2cc([C@H]3CC[C@@H](Oc4nscc4C4CC4)C3)n[nH]2)n(C)n1] | ¹H NMR (400 MHz, DMSO) 12.24 (s, 1H), 10.71 (s, 1H), 8.26 (s, 1H), 7.11 (s, 1H), 6.47 (s, 1H), 5.35 (s, 1H), 4.33 (s, 2H), 4.04 (s, 3H), 3.27 (s, 3H), 3.18 (q, J = 8.3 Hz, 1H), 2.62 (dt, J = 14.5, 7.6 Hz, 1H), 2.15-1.93 (m, 1H), 1.93- 1.70 (m, 3H), 0.86 (dt, J = 8.6, 3.1 Hz, 2H), 0.70-0.59 (m, 2H). | 443.15 | |
| N-(5-((1s,3s)-3-(((4- isopropylpyridazin-3- yl)oxy)methyl)cyclo- butyl)-1H-pyrazol-3- yl)-2-(3- methylisoxazol-5- yl)acetamide | 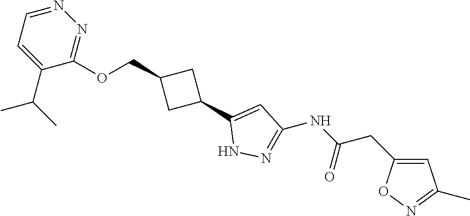 [SMILES: Cc1cc(CC(=O)Nc2cc([C@H]3C[C@@H](COc4nnccc4C(C)C)C3)[nH]n2)on1] | ¹H NMR (400 MHz, DMSO-d₆) 12.15 (s, 1H), 10.64 (s, 1H), 8.77 (d, J = 4.7 Hz, 1H), 7.42 (d, J = 4.7 Hz, 1H), 6.38 (s, 1H), 6.22 (s, 1H), 4.42 (d, J = 5.7 Hz, 2H), 3.84 (s, 2H), 3.42 (q, J = 9.0 Hz, 1H), 3.08 (hept, J = 6.9 Hz, 1H), 2.79 (dh, J = 20.7, 6.9 Hz, 1H), 2.43 (qd, J = 8.3, 2.6 Hz, 2H), 2.20 (s, 3H), 2.07 (qd, J = 9.6, 2.4 Hz, 2H), 1.19 (d, J = 6.9 Hz, 6H). | 411.25 | |
| N-(5-((1r,3r)-3-(((4- isopropylpyridazin-3- yl)oxy)methyl)cyclo- butyl)-1H-pyrazol-3- yl)-2-(3- methylisoxazol-5- yl)acetamide | 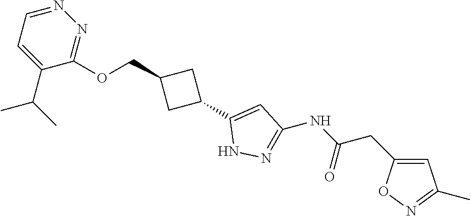 [SMILES: Cc1cc(CC(=O)Nc2cc([C@H]3C[C@H](COc4nnccc4C(C)C)C3)[nH]n2)on1] | ¹H NMR (400 MHz, DMSO-d₆) 12.17 (s, 1H), 10.66 (s, 1H), 8.79 (d, J = 4.7 Hz, 2H), 7.45 (d, J = 4.7 Hz, 2H), 6.38 (s, 2H), 6.23 (s, 2H), 4.56 (d, J = 6.8 Hz, 4H), 3.84 (s, 3H), 3.62 (p, J = 8.0 Hz, 2H), 3.09 (hept, J = 6.9 Hz, 2H), 2.84 (p, J = 7.0, 6.6 Hz, 2H), 2.34-2.23 (m, 7H), 2.21 (s, 5H), 1.21 (d, J = 6.8 Hz, 7H), 1.19 (s, 4H). | 411.25 | |
| N-(5-((1r,3r)-3-(((4- isopropylpyridazin-3- yl)oxy)methyl)cyclo- butyl)-1H-pyrazol-3- yl)-3- (methoxymethyl)-1- methyl-1H-pyrazole- 5-carboxamide | 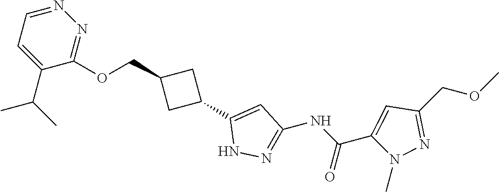 [SMILES: COCc1cc(C(=O)Nc2cc([C@H]3C[C@H](COc4nnccc4C(C)C)C3)[nH]n2)n(C)n1] | ¹H NMR (400 MHz, DMSO-d₆) 12.28 (d, J = 2.1 Hz, 1H), 10.75 (s, 1H), 8.80 (d, J = 4.7 Hz, 1H), 7.46 (dd, J = 4.7, 0.8 Hz, 1H), 7.14 (s, 1H), 6.53 (d, J = 2.3 Hz, 1H), 4.58 (d, J = 6.8 Hz, 2H), 4.34 (s, 2H), 4.07 (s, 3H), 3.66 (p, J = 8.2 Hz, 1H), 3.28 (s, 3H), 3.10 (h, J = 6.9 Hz, 1H), 2.88 (p, J = 6.9 Hz, 1H), 2.34 (t, J = 7.5 Hz, 4H), 1.22 (d, J = 6.9 Hz, 6H). | 440.20 | |
| N-(5-((1s,3s)-3-(((4- isopropylpyridazin-3- yl)oxy)methyl)cyclo- butyl)-1H-pyrazol-3- yl)-3- (methoxymethyl)-1- methyl-1H-pyrazole- 5-carboxamide | 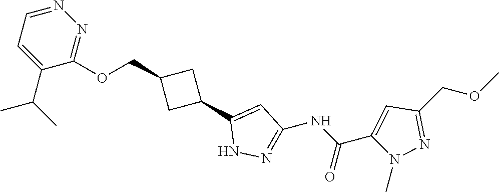 [SMILES: COCc1cc(C(=O)Nc2cc([C@H]3C[C@@H](COc4nnccc4C(C)C)C3)[nH]n2)n(C)n1] | ¹H NMR (400 MHz, DMSO-d₆) 12.26 (s, 1H), 10.75 (s, 1H), 8.78 (d, J = 4.7 Hz, 1H), 7.45 (d, J = 4.7 Hz, 1H), 7.13 (s, 1H), 6.53 (s, 1H), 4.45 (d, J = 5.6 Hz, 2H), 4.34 (s, 2H), 4.07 (s, 3H), 3.51-3.37 (m, 1H), 3.27 (s, 3H), 3.13 (hept, J = 7.0 Hz, 1H), 2.88-2.74 (m, 1H), 2.45 (td, J = 8.4, 2.7 Hz, 2H), 2.12 (qd, J = 9.6, 2.6 Hz, 2H), 1.22 (d, J = 6.8 Hz, 6H). | 440.20 | |
| rel-N-(5-((1R,3S)-3- ((4-cyclo- propylisothiazol- 3- yl)oxy)cyclopentyl)- 1H-pyrazol-3- yl)acetamide | 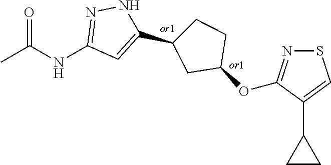 [SMILES: CC(=O)Nc1cc([C@H]2CC[C@@H](Oc3nscc3C3CC3)C2)[nH]n1] | ¹H NMR (400 MHz, Chloroform-d) 8.06 (s, 1H), 7.87 (d, J = 0.9 Hz, 1H), 6.52 (s, 1H), 5.54 (dt, J = 5.7, 2.9 Hz, 1H), 3.41 (dt, J = 15.6, 8.0 Hz, 1H), 2.60 (ddd, J = 15.1, 9.7, 5.8 Hz, 1H), 2.32-2.17 (m, 2H), 2.18 (s, 3H), 2.07 (tdd, J = 12.8, 9.5, 5.6 Hz, 2H), 2.03-1.89 (m, 1H), 1.79 (ddd, J = 13.6, 8.5, 5.2 Hz, 1H), 1.06-0.93 (m, 2H), 0.66 (tt, J = 5.0, 2.6 Hz, 2H). | 333.05 | |
| rel-N-(5-((1R,3S)-3- ((4-cyclo- propylisothiazol- 3- yl)oxy)cyclopentyl)- 1H-pyrazol-3- yl)acetamide | 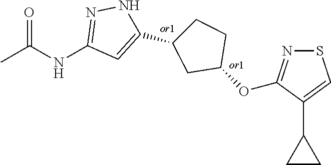 [SMILES: CC(=O)Nc1cc([C@@H]2CC[C@H](Oc3nscc3C3CC3)C2)[nH]n1] | ¹H NMR (400 MHz, Chloroform-d) 8.06 (s, 1H), 7.87 (d, J = 0.9 Hz, 1H), 6.52 (s, 1H), 5.54 (tt, J = 5.4, 2.5 Hz, 1H), 3.47-3.34 (m, 1H), 2.60 (ddd, J = 15.1, 9.6, 5.9 Hz, 1H), 2.31-2.17 (m, 2H), 2.18 (s, 3H), 2.15-1.99 (m, 2H), 2.03- 1.89 (m, 1H), 1.84-1.73 (m, 1H), 1.06-0.93 (m, 2H), 0.73-0.60 (m, 2H). | 333.10 | |
| N-(5-((1r,3r)-3-(((4- isopropylpyridazin-3- yl)oxy)methyl)cyclo- butyl)-1H-pyrazol-3- yl)thiazolo[5,4- c]pyridin-4-amine | 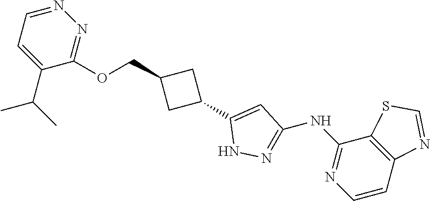 [SMILES: CC(C)c1ccnnc1OC[C@H]1C[C@H](c2cc(Nc3nccc4ncsc34)n[nH]2)C1] | ¹H NMR (400 MHz, DMSO-d6) 12.20 (s, 1H), 9.48 (s, 2H), 8.80 (d, J = 4.7 Hz, 1H), 8.15 (d, J = 5.7 Hz, 1H), 7.46 (dd, J = 4.8, 0.8 Hz, 1H), 7.41 (d, J = 5.7 Hz, 1H), 6.43 (s, 1H), 4.59 (d, J = 6.8 Hz, 2H), 3.67 (p, J = 8.0 Hz, 1H), 3.11 (hept, J = 6.9 Hz, 1H), 2.88 (hept, J = 7.1 Hz, 1H), 2.43-2.28 (m, 4H), 1.22 (d, J = 6.9 Hz, 6H). | 422.35 | |
| N-(3-((1s,3s)-3-(((4- isopropylpyridazin-3- yl)oxy)methyl)cyclo- butyl)-1H-pyrazol-5- yl)thiazolo[5,4- c]pyridin-4-amine | 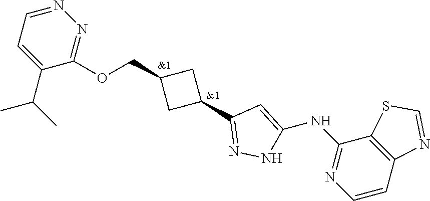 [SMILES: CC(C)c1ccnnc1OC[C@H]1C[C@@H](c2cc(Nc3nccc4ncsc34)[nH]n2)C1] | ¹H NMR (400 MHz, DMSO-d₆) 12.17 (s, 1H), 9.53 (s, 1H), 9.48 (s, 1H), 8.78 (d, J = 4.8 Hz, 1H), 8.14 (d, J = 5.7 Hz, 1H), 7.46-7.38 (m, 2H), 6.40 (s, 1H), 4.46 (d, J = 5.9 Hz, 2H), 3.45 (p, J = 9.0 Hz, 1H), 3.09 (hept, J = 6.8 Hz, 1H), 2.75 (m, 1H), 2.45 (td, J = 8.4, 2.8 Hz, 2H), 2.18-2.06 (m, 2H), 1.19 (d, J = 6.9 Hz, 6H).. | 422.35 | |
| N-(5-((1s,3s)-3-(((4- cyclopropylisothiazol- 3- yl)oxy)methyl)cyclo- butyl)-1H-pyrazol-3- yl)-3- (methoxymethyl)-1- methyl-1H-pyrazole- 5-carboxamide | 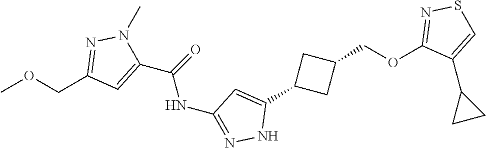 [SMILES: COCc1cc(C(=O)Nc2cc([C@H]3C[C@@H](COc4nscc4C4CC4)C3)[nH]n2)n(C)n1] | ¹H NMR (400 MHz, Chloroform-d) 10.88 (s, 1H), 7.83 (d, J = 0.7 Hz, 1H), 6.96 (s, 1H), 6.86 (s, 1H), 4.53 (s, 2H), 4.49 (d, J = 6.6 Hz, 2H), 4.16 (s, 3H), 3.59 (q, J = 7.9 Hz, 1H), 3.53 (s, 3H), 2.94 (hept, J = 6.9 Hz, 1H), 2.40 (t, J = 7.5 Hz, 4H), 1.82 (tt, J = 8.5, 5.0 Hz, 1H), 1.01- 0.87 (m, 2H), 0.80-0.61 (m, 2H). | 443.10 | |
| N-(5-((1r,3r)-3-(((4- cyclopropylisothiazol- 3- yl)oxy)methyl)cyclo- butyl)-1H-pyrazol-3- yl)-3- (methoxymethyl)-1- methyl-1H-pyrazole- 5-carboxamide | 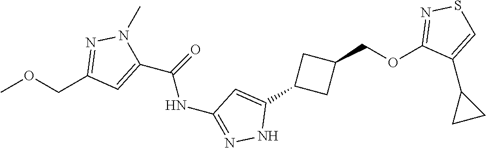 [SMILES: COCc1cc(C(=O)Nc2cc([C@H]3C[C@H](COc4nscc4C4CC4)C3)[nH]n2)n(C)n1] | ¹H NMR (400 MHz, Chloroform-d) 10.76 (s, 1H), 7.82 (s, 1H), 6.93 (s, 1H), 6.80 (s, 1H), 4.54 (s, 2H), 4.38 (d, J = 5.8 Hz, 2H), 4.16 (s, 3H), 3.54 (s, 3H), 3.43-3.21 (m, 1H), 2.96- 2.73 (m, 1H), 2.64-2.42 (m, 2H), 2.19 (qd, J = 9.6, 2.6 Hz, 2H), 1.93-1.71 (m, 1H), 1.10- 0.83 (m, 2H), 0.79-0.52 (m, 2H). | 443.15 | |
| rel-N-(5-((2R,4R)-4- ((4- isopropylpyridazin-3- yl)oxy)tetrahydrofuran- 2-yl)-1H-pyrazol-3- yl)pyrazolo[1,5- a]pyrazin-4-amine | 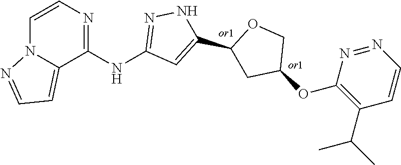 [SMILES: CC(C)c1ccnnc1O[C@@H]1CO[C@H](c2cc(Nc3nccn4nccc34)n[nH]2)C1] | ¹H NMR (400 MHz, DMSO-d₆) 12.41 (s, 1H), 10.08 (s, 1H), 8.78 (d, J = 4.8 Hz, 1H), 8.09 (s, 1H), 7.97 (s, 1H), 7.43 (dd, J = 4.8, 0.7 Hz, 1H), 7.36 (d, J = 4.9 Hz, 2H), 6.84 (s, 1H), 5.81 (dd, J = 6.4, 3.6 Hz, 1H), 5.08 (s, 1H), 4.16- 4.05 (m, 2H), 2.98 (p, J = 6.8 Hz, 1H), 2.87 (dt, J = 14.3, 7.4 Hz, 1H), 2.34-2.25 (m, 1H), 1.09 (dd, J = 6.9, 2.3 Hz, 6H). | 407.10 | |
| rel-N-(5-((2R,4R)-4- ((4- isopropylpyridazin-3- yl)oxy)tetrahydrofuran- 2-yl)-1H-pyrazol-3- yl)pyrazolo[1,5- a]pyrazin-4-amine | 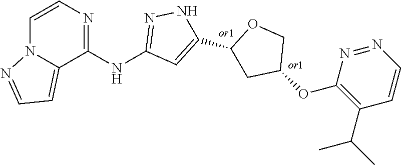 [SMILES: CC(C)c1ccnnc1O[C@H]1CO[C@@H](c2cc(Nc3nccn4nccc34)n[nH]2)C1] | ¹H NMR (400 MHz, DMSO-d₆) 12.41 (s, 1H), 10.07 (s, 1H), 8.78 (d, J = 4.7 Hz, 1H), 8.09 (s, 1H), 8.00-7.95 (m, 1H), 7.43 (d, J = 4.8 Hz, 1H), 7.35 (s, 2H), 6.84 (s, 1H), 5.81 (d, J = 2.7 Hz, 1H), 5.09 (s, 1H), 4.10 (s, 2H), 2.98 (p, J = 6.8 Hz, 1H), 2.87 (dt, J = 14.2, 7.4 Hz, 1H), 2.34-2.25 (m, 1H), 1.09 (dd, J = 6.9, 2.3 Hz, 6H). | 407.10 | |
| ((1s,3s)-3-(3- (thiazolo[5,4- c]pyridin-4-ylamino)- 1H-pyrazol-5- yl)cyclobutyl)methyl isopropylcarbamate | 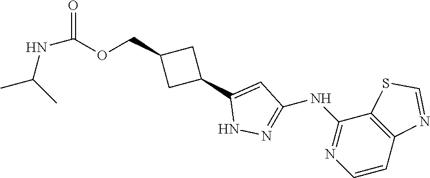 [SMILES: CC(C)NC(=O)OC[C@H]1C[C@@H](c2cc(Nc3nccc4ncsc34)n[nH]2)C1] | ¹H NMR (400 MHz) 9.02 (s, 1H), 8.27 (d, J = 5.7 Hz, 1H), 7.52 (d, J = 5.7 Hz, 2H), 6.49 (s, 1H), 4.74 (s, 1H), 4.11 (s, 2H), 3.86 (m, 1H), 3.49 (p, J = 9.0 Hz, 1H), 2.63 (s, 1H), 2.52 (t, J = 9.3 Hz, 2H), 2.14 (m, 2H), 1.17 (d, J = 6.5 Hz, 6H). | 387.30 | |
| ((1r,3r)-3-(3- (thiazolo[5,4- c]pyridin-4-ylamino)- 1H-pyrazol-5- yl)cyclobutyl)methyl isopropylcarbamate | 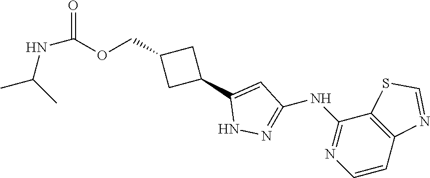 [SMILES: CC(C)NC(=O)OC[C@H]1C[C@H](c2cc(Nc3nccc4ncsc34)n[nH]2)C1] | ¹H NMR (400 MHz) 9.05 (s, 1H), 8.27 (d, J = 5.7 Hz, 1H), 8.03 (s, 1H), 7.53 (d, J = 5.7 Hz, 1H), 6.45 (s, 1H), 4.68 (d, J = 7.7 Hz, 1H), 4.17 (d, J = 7.0 Hz, 2H), 3.84 (s, 1H), 3.60 (p, J = 8.0 Hz, 1H), 2.76 (s, 1H), 2.36 (q, J = 8.6 Hz, 2H), 2.27 (s, 2H), 1.18 (d, J = 6.5 Hz, 6H). | 387.30 | |
| rel-(3R,5R)-5-(5-((2- (methoxymethyl) thiazolo[5,4-c]pyridin-4- yl)amino)-1H- pyrazol-3- yl)tetrahydrofuran-3- yl isopropylcarbamate | 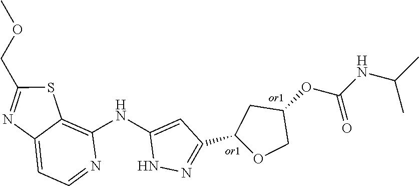 [SMILES: COCc1nc2ccnc(Nc3cc([C@@H]4C[C@H](OC(=O)NC(C)C)CO4)n[nH]3)c2s1] | ¹H NMR (400 MHz, DMSO-d₆) 12.35 (s, 1H), 9.49 (s, 1H), 8.15 (d, J = 5.6 Hz, 1H), 7.34 (s, 1H), 7.12 (d, J = 7.7 Hz, 1H), 6.62 (d, J = 69.4 Hz, 1H), 5.18 (s, 1H), 4.87 (s, 3H), 3.88 (d, J = 10.7 Hz, 2H), 3.59 (h, J = 6.8 Hz, 1H), 3.48 (s, 3H), 2.68 (p, J = 1.9 Hz, 1H), 1.98 (s, 1H), 1.04 (dd, J = 6.6, 4.4 Hz, 6H). | 433.10 | |
| rel-(3R,5R)-5-(5-((2- (methoxymethyl) thiazolo[5,4-c]pyridin-4- yl)amino)-1H- pyrazol-3- yl)tetrahydrofuran-3- yl isopropylcarbamate | 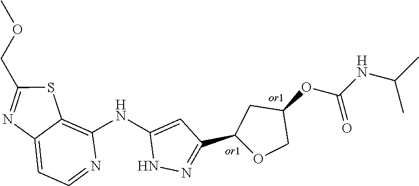 [SMILES: COCc1nc2ccnc(Nc3cc([C@H]4C[C@@H](OC(=O)NC(C)C)CO4)n[nH]3)c2s1] | ¹H NMR (400 MHz, DMSO-d₆) 12.36 (s, 1H), 9.52 (s, 1H), 8.15 (d, J = 5.6 Hz, 1H), 7.34 (d, J = 5.6 Hz, 1H), 7.12 (d, J = 7.7 Hz, 1H), 6.49 (s, 1H), 5.18 (s, 1H), 4.87 (s, 3H), 3.86 (q, J = 7.5, 4.7 Hz, 2H), 3.59 (h, J = 6.7 Hz, 1H), 3.48 (s, 3H), 2.68 (p, J = 1.9 Hz, 1H), 1.98 (s, 1H), 1.04 (dd, J = 6.6, 4.5 Hz, 6H). | 433.15 | |
| N-(5-((1r,3r)-3-(((4- isopropylpyridazin-3- yl)oxy)methyl)cyclo- butyl)-1H-pyrazol-3- yl)pyrazolo[1,5- a]pyrazin-4-amine | 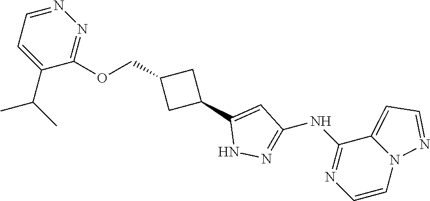 [SMILES: CC(C)c1ccnnc1OC[C@H]1C[C@H](c2cc(Nc3nccn4nccc34)n[nH]2)C1] | ¹H NMR (400 MHz, DMSO-d₆) 12.18 (s, 1H), 10.02 (s, 1H), 8.80 (d, J = 4.7 Hz, 1H), 8.12- 8.05 (m, 1H), 7.96 (d, J = 2.3 Hz, 1H), 7.46 (dd, J = 4.8, 0.8 Hz, 1H), 7.40 (d, J = 4.8 Hz, 1H), 7.34 (d, J = 2.2 Hz, 1H), 6.71 (s, 1H), 4.59 (d, J = 6.8 Hz, 2H), 3.69 (p, J = 8.2 Hz, 1H), 3.11 (hept, J = 6.9 Hz, 1H), 2.88 (dt, J = 13.8, 6.8 Hz, 1H), 2.43-2.25 (m, 4H), 1.22 (d, J = 6.9 Hz, 6H). | 405.25 | 4 |
| N-(5-((1s,3s)-3-(((4- isopropylpyridazin-3- yl)oxy)methyl)cyclo- butyl)-1H-pyrazol-3- yl)pyrazolo[1,5- a]pyrazin-4-amine | 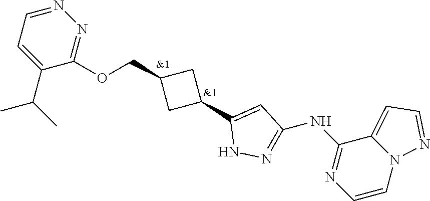 [SMILES: CC(C)c1ccnnc1OC[C@H]1C[C@@H](c2cc(Nc3nccn4nccc34)n[nH]2)C1] | ¹H NMR (400 MHz, DMSO-d₆) 12.18 (s, 1H), 10.02 (s, 1H), 8.80 (d, J = 4.7 Hz, 1H), 8.12- 8.05 (m, 1H), 7.96 (d, J = 2.3 Hz, 1H), 7.46 (dd, J = 4.8, 0.8 Hz, 1H), 7.40 (d, J = 4.8 Hz, 1H), 7.34 (d, J = 2.2 Hz, 1H), 6.71 (s, 1H), 4.59 (d, J = 6.8 Hz, 2H), 3.69 (p, J = 8.2 Hz, 1H), 3.11 (hept, J = 6.9 Hz, 1H), 2.88 (dt, J = 13.8, 6.8 Hz, 1H), 2.43-2.25 (m, 4H), 1.22 (d, J = 6.9 Hz, 6H). | 405.15 | −14 |
| rel-N-(3-((2R,4R)-4- ((4- isopropylpyridazin-3- yl)oxy)tetrahydrofuran- 2-yl)-1H-pyrazol-5- yl)-2- (methoxymethyl) pyrazolo[1,5-a]pyrazin- 4-amine | 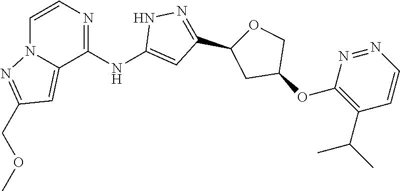 [SMILES: COCc1cc2c(Nc3cc([C@@H]4C[C@H](Oc5nnccc5C(C)C)CO4)n[nH]3)nccn2n1] | ¹H NMR (400 MHz, DMSO-d₆) 12.41 (s, 1H), 10.07 (s, 1H), 8.78 (d, J = 4.8 Hz, 1H), 8.04 (s, 1H), 7.43 (d, J = 4.8 Hz, 1H), 7.35 (d, J = 4.8 Hz, 1H), 7.28 (s, 1H), 6.81 (s, 1H), 5.84-5.77 (m, 1H), 5.08 (s, 1H), 4.57 (s, 2H), 4.10 (t, J = 3.4 Hz, 2H), 3.33 (s, 3H), 2.97 (p, J = 6.9 Hz, 1H), 2.86 (dt, J = 14.3, 7.5 Hz, 1H), 2.30 (ddd, J = 13.7, 6.1, 2.6 Hz, 1H), 1.09 (dd, J = 6.9, 2.5 Hz, 6H). | 451.20 | 30 |
| rel-N-(3-((2R,4R)-4- ((4- isopropylpyridazin-3- yl)oxy)tetrahydrofuran- 2-yl)-1H-pyrazol-5- yl)-2- (methoxymethyl) pyrazolo[1,5-a] pyrazin-4-amine | 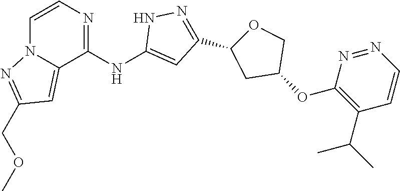 [SMILES: COCc1cc2c(Nc3cc([C@H]4C[C@@H](Oc5nnccc5C(C)C)CO4)n[nH]3)nccn2n1] | ¹H NMR (400 MHz, DMSO-d₆) 12.41 (s, 1H), 10.08 (s, 1H), 8.78 (d, J = 4.8 Hz, 1H), 8.03 (s, 1H), 7.43 (d, J = 4.8 Hz, 1H), 7.35 (d, J = 4.8 Hz, 1H), 7.28 (s, 1H), 6.80 (s, 1H), 5.80 (ddt, J = 6.8, 4.6, 2.5 Hz, 1H), 5.07 (s, 1H), 4.57 (s, 2H), 4.14-4.06 (m, 2H), 3.33 (s, 3H), 2.97 (dt, J = 13.9, 7.0 Hz, 1H), 2.86 (dt, J = 14.3, 7.5 Hz, 1H), 2.30 (ddd, J = 13.7, 6.1, 2.6 Hz, 1H), 1.09 (dd, J = 6.9, 2.5 Hz, 6H). | 451.20 | −20 |
| rel-N-(5-((2R,4R)-4- ((4- isopropylpyridazin-3- yl)oxy)tetrahydrofuran- 2-yl)-1H-pyrazol-3- yl)thiazolo[5,4- c]pyridin-4-amine | 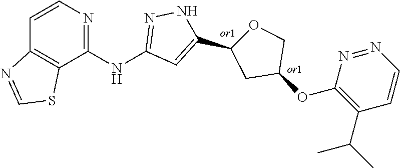 [SMILES: CC(C)c1ccnnc1O[C@@H]1CO[C@H](c2cc(Nc3nccc4ncsc34)n[nH]2)C1] | ¹H NMR (400 MHz, DMSO-d₆) 12.38 (s, 1H), 9.55 (d, J = 41.5 Hz, 2H), 8.78 (d, J = 4.8 Hz, 1H), 8.14 (s, 1H), 7.43 (d, J = 4.7 Hz, 2H), 6.61 (s, 1H), 5.84-5.69 (m, 1H), 5.05 (s, 1H), 4.09 (s, 2H), 2.98 (p, J = 6.9 Hz, 1H), 2.86 (s, 1H), 2.33-2.25 (m, 1H), 1.09 (s, 6H). | 424.15 | 31 |
| rel-N-(5-((2R,4R)-4- ((4- isopropylpyridazin-3- yl)oxy)tetrahydrofuran- 2-yl)-1H-pyrazol-3- yl)thiazolo[5,4- c]pyridin-4-amine | 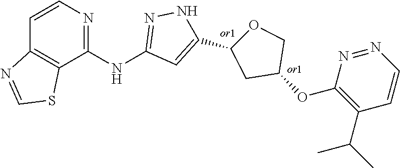 [SMILES: CC(C)c1ccnnc1O[C@H]1CO[C@@H](c2cc(Nc3nccc4ncsc34)n[nH]2)C1] | ¹H NMR (400 MHz, DMSO-d₆) 12.39 (s, 1H), 9.55 (d, J = 39.3 Hz, 2H), 8.78 (d, J = 4.8 Hz, 1H), 8.14 (s, 1H), 7.43 (d, J = 4.8 Hz, 2H), 6.59 (s, 1H), 5.80 (dq, J = 6.5, 3.1 Hz, 1H), 5.04 (s, 1H), 4.09 (s, 2H), 2.98 (p, J = 6.9 Hz, 1H), 2.87 (dd, J = 14.1, 7.4 Hz, 1H), 2.29 (ddd, J = 13.8, 6.6, 2.7 Hz, 1H), 1.09 (d, J = 2.9 Hz, 6H). | 424.15 | −22 |
| rac-N-(5-((1s,3s)-3- (((4- isopropylpyridazin-3- yl)oxy)methyl)cyclo- butyl)-1H-pyrazol-3- yl)-2- (methoxymethyl) pyrazolo[1,5-a]pyrazin-4- amine | 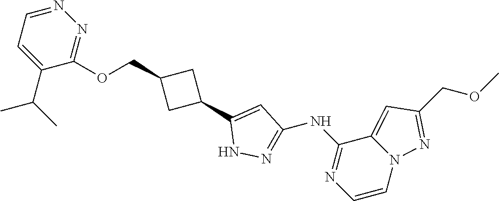 [SMILES: COCc1cc2c(Nc3cc([C@H]4C[C@@H](COc5nnccc5C(C)C)C4)[nH]n3)nccn2n1] | ¹H NMR (400 MHz, DMSO-d₆) 12.17 (s, 1H), 10.01 (s, 1H), 8.78 (d, J = 4.7 Hz, 1H), 8.02 (d, J = 4.8 Hz, 1H), 7.43 (d, J = 4.8 Hz, 1H), 7.37 (d, J = 4.8 Hz, 1H), 7.30 (s, 1H), 6.67 (s, 1H), 4.56 (s, 2H), 4.46 (d, J = 5.7 Hz, 2H), 3.46 (p, J = 9.1, 8.6 Hz, 1H), 3.33 (s, 3H), 3.10 (p, J = 6.9 Hz, 1H), 2.81 (p, J = 7.7, 6.3 Hz, 1H), 2.48- 2.39 (m, 2H), 2.13 (qd, J = 9.8, 2.6 Hz, 2H), 1.20 (d, J = 6.9 Hz, 6H). | 449.20 | |
| rac-N-(5-((1r,3r)-3- (((4- isopropylpyridazin-3- yl)oxy)methyl)cyclo- butyl)-1H-pyrazol-3- yl)-2- (methoxymethyl) pyrazolo[1,5-a]pyrazin-4- amine | 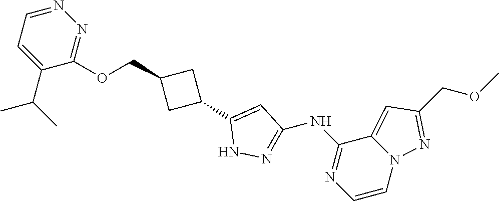 [SMILES: COCc1cc2c(Nc3cc([C@H]4C[C@H](COc5nnccc5C(C)C)C4)[nH]n3)nccn2n1] | ¹H NMR (400 MHz, DMSO-d₆) 12.18 (s, 1H), 10.02 (s, 1H), 8.80 (d, J = 4.7 Hz, 1H), 8.02 (d, J = 4.7 Hz, 1H), 7.46 (dd, J = 4.7, 0.8 Hz, 1H), 7.40 (d, J = 4.8 Hz, 1H), 7.29 (s, 1H), 6.70 (s, 1H), 4.64-4.54 (m, 4H), 3.69 (p, J = 8.1 Hz, 1H), 3.33 (s, 3H), 3.11 (p, J = 6.9 Hz, 1H), 2.88 (dt, J = 13.5, 6.9 Hz, 1H), 2.42-2.28 (m, 4H), 1.22 (d, J = 6.9 Hz, 6H). | 449.20 | |
| rel-3- (methoxymethyl)-1- methyl-N-(5- ((1R,3S)-3-(6-methyl- 2-oxo-1,2- dihydropyridin-3- yl)cyclopentyl)-1H- pyrazol-3-yl)-1H- pyrazole-5- carboxamide | 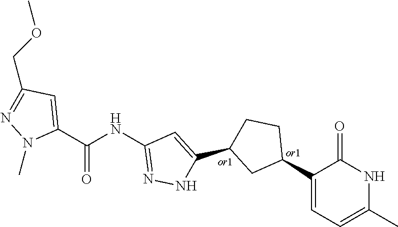 [SMILES: COCc1cc(C(=O)Nc2cc([C@H]3CC[C@@H](c4ccc(C)[nH]c4=O)C3)[nH]n2)n(C)n1] | ¹H NMR (400 MHz, DMSO-d₆) 12.27 (s, 1H), 11.49 (s, 1H), 10.73 (s, 1H), 7.24 (d, J = 6.9 Hz, 1H), 7.12 (s, 1H), 6.43 (s, 1H), 6.05-5.88 (m, 1H), 4.34 (s, 2H), 4.06 (s, 3H), 3.27 (s, 3H), 3.15 (td, J = 18.3, 17.9, 10.1 Hz, 2H), 2.26 (dt, J = 12.9, 6.8 Hz, 1H), 2.18-2.12 (m, 3H), 2.09 (q, J = 7.2, 6.4 Hz, 1H), 2.05-1.95 (m, 1H), 1.79 (dt, J = 12.1, 8.8 Hz, 1H), 1.74-1.63 (m, 2H). | 411.20 | |
| rel-3- (methoxymethyl)-1- methyl-N-(5- ((1R,3S)-3-(6-methyl- 2-oxo-1,2- dihydropyridin-3- yl)cyclopentyl)-1H- pyrazol-3-yl)-1H- pyrazole-5- carboxamide | 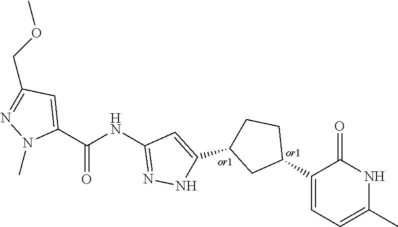 [SMILES: COCc1cc(C(=O)Nc2cc([C@@H]3CC[C@H](c4ccc(C)[nH]c4=O)C3)[nH]n2)n(C)n1] | ¹H NMR (400 MHz, DMSO-d₆) 12.27 (s, 1H), 11.49 (s, 1H), 10.73 (s, 1H), 7.24 (d, J = 6.9 Hz, 1H), 7.12 (s, 1H), 6.43 (s, 1H), 5.93 (d, J = 6.9 Hz, 1H), 4.34 (s, 2H), 4.06 (s, 3H), 3.27 (s, 3H), 3.25-3.10 (m, 2H), 2.26 (dt, J = 12.8, 6.8 Hz, 1H), 2.14 (s, 3H), 2.11-2.06 (m, 1H), 2.06-1.95 (m, 1H), 1.79 (ddd, J = 11.6, 9.1, 4.6 Hz, 1H), 1.75-1.63 (m, 2H). | 411.20 | |
| rel-3- (methoxymethyl)-1- methyl-N-(5- ((1R,3S)-3-((5- methylthiazol-2-yl) methyl)cyclopentyl)- 1H-pyrazol-3-yl)- 1H-pyrazole-5- carboxamide | 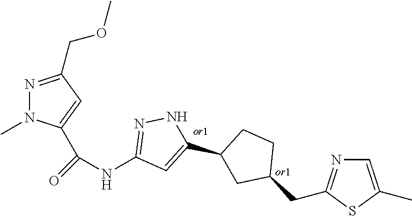 | ¹H NMR (400 MHz, DMSO-d₆) 12.19 (s, 1H), 10.71 (s, 1H), 7.44-7.27 (m, 1H), 7.12 (s, 1H), 6.40 (s, 1H), 4.34 (s, 2H), 4.05 (s, 3H), 3.27 (s, 3H), 3.08 (t, J = 9.1 Hz, 1H), 2.97 (d, J = 7.3 Hz, 2H), 2.40 (d, J = 1.3 Hz, 3H), 2.37 (d, J = 8.3 Hz, 1H), 2.20 (dt, J = 13.3, 7.0 Hz, 1H), 2.02 (dt, J = 13.2, 6.8 Hz, 1H), 1.85 (dq, J = 15.3, 7.7 Hz, 1H), 1.75-1.63 (m, 1H), 1.57-1.43 (m, 1H), 1.36 (g, J = 10.9 Hz, 1H). | 415.25 | |
| rel-3- (methoxymethyl)-1- methyl-N-(5- ((1R,3S)-3-((5- methylthiazol-2-yl) methyl)cyclopentyl)- 1H-pyrazol-3-yl)- 1H-pyrazole-5- carboxamide | 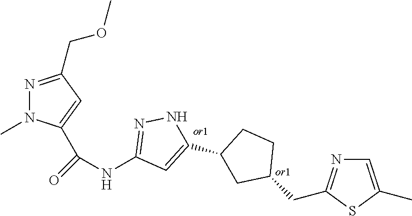 | ¹H NMR (400 MHz, DMSO-d₆) 12.19 (s, 1H), 10.71 (s, 1H), 7.34 (q, J = 1.2 Hz, 1H), 7.11 (s, 1H), 6.39 (s, 1H), 4.34 (s, 2H), 4.05 (s, 3H), 3.27 (s, 3H), 3.14-3.03 (m, 1H), 2.97 (d, J = 7.3 Hz, 2H), 2.40 (d, J = 1.2 Hz, 3H), 2.37 (d, J = 9.8 Hz, 1H), 2.20 (dt, J = 13.3, 7.0 Hz, 1H), 2.02 (dt, J = 13.4, 6.8 Hz, 1H), 1.85 (dq, J = 15.3, 7.7 Hz, 1H), 1.74-1.63 (m, 1H), 1.54-1.44 (m, 1H), 1.35 (q, J = 10.7 Hz, 1H). | 415.10 | |
| rel-3- (methoxymethyl)-1- methyl-N-(5- ((1R,3R)-3-((5- methylthiazol-2-yl) methyl)cyclopentyl)- 1H-pyrazol-3-yl)- 1H-pyrazole-5- carboxamide | 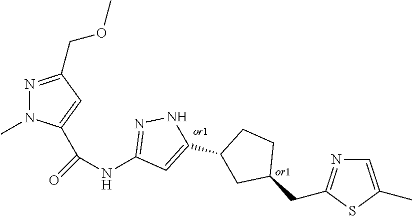 | ¹H NMR (400 MHz, DMSO-d₆) 12.18 (s, 1H), 10.71 (s, 1H), 7.34 (q, J = 1.2 Hz, 1H), 7.11 (s, 1H), 6.38 (s, 1H), 4.34 (s, 2H), 4.05 (s, 3H), 3.27 (s, 3H), 3.20 (t, J = 7.9 Hz, 1H), 2.95 (d, J = 7.5 Hz, 2H), 2.48-2.43 (m, 1H), 2.40 (d, J = 1.3 Hz, 3H), 2.08 (dd, J = 8.0, 4.0 Hz, 1H), 1.93 (ddd, J = 11.1, 9.4, 5.3 Hz, 1H), 1.83 (dq, J = 15.3, 7.7 Hz, 2H), 1.69-1.59 (m, 1H), 1.37 (dq, J = 12.7, 8.3 Hz, 1H). | 415.15 | |
| rel-3- (methoxymethyl)-1- methyl-N-(5- ((1R,3R)-3-((5- methylthiazol-2-yl) methyl)cyclopentyl)- 1H-pyrazol-3-yl)- 1H-pyrazole-5- carboxamide | 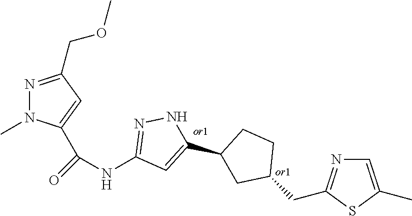 | ¹H NMR (400 MHz, DMSO-d₆) 12.19 (s, 1H), 10.71 (s, 1H), 7.34 (q, J = 1.3 Hz, 1H), 7.11 (s, 1H), 6.38 (s, 1H), 4.34 (s, 2H), 4.05 (s, 3H), 3.19 (q, J = 7.6 Hz, 1H), 2.95 (d, J = 7.5 Hz, 2H), 2.49-2.42 (m, 1H), 2.40 (d, J = 1.3 Hz, 3H), 2.13-2.05 (m, 1H), 1.93 (dtd, J = 11.2, 7.6, 3.4 Hz, 1H), 1.83 (dq, J = 15.4, 7.6 Hz, 2H), 1.64 (dq, J = 12.3, 8.5 Hz, 1H), 1.37 (dq, J = 12.7, 8.0 Hz, 1H). | 415.05 | |
| rel-N-(3-((1R,3S)-3- (5-isopropylpyridin- 2-yl)cyclopentyl)-1H- pyrazol-5-yl)-3- (methoxymethyl)-1- methyl-1H-pyrazole- 5-carboxamide | 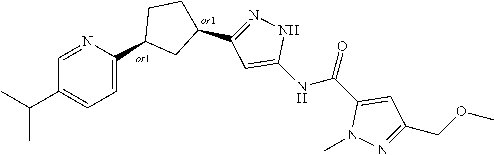 [SMILES: COCc1cc(C(=O)Nc2cc([C@@H]3CC[C@H](c4ccc(C(C)C)cn4)C3)n[nH]2)n(C)n1] | ¹H NMR (400 MHz, DMSO-d₆) 12.29 (s, 1H), 10.73 (s, 1H), 8.41 (d, J = 2.4 Hz, 1H), 7.59 (dd, J = 8.1, 2.4 Hz, 1H), 7.25 (d, J = 8.0 Hz, 1H), 7.12 (s, 1H), 6.46 (s, 1H), 4.34 (s, 2H), 4.06 (s, 3H), 3.36 (s, 1H), 3.27 (s, 4H), 2.91 (p, J = 6.9 Hz, 1H), 2.39 (dt, J = 13.0, 6.9 Hz, 1H), 2.24-2.09 (m, 2H), 1.89 (dddd, J = 32.7, 14.9, 9.7, 5.2 Hz, 3H), 1.22 (d, J = 7.0 Hz, 6H). | 423.30 | |
| rel-N-(3-((1R,3S)-3- (5-isopropylpyridin- 2-yl)cyclopentyl)-1H- pyrazol-5-yl)-3- (methoxymethyl)-1- methyl-1H-pyrazole- 5-carboxamide | 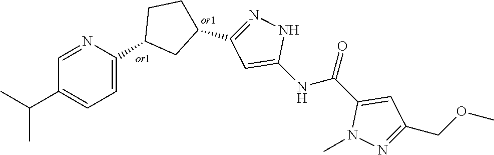 [SMILES: COCc1cc(C(=O)Nc2cc([C@H]3CC[C@@H](c4ccc(C(C)C)cn4)C3)n[nH]2)n(C)n1] | ¹H NMR (400 MHz, DMSO-d₆) 12.29 (s, 1H), 10.73 (s, 1H), 8.41 (d, J = 2.4 Hz, 1H), 7.59 (dd, J = 8.1, 2.4 Hz, 1H), 7.25 (d, J = 8.0 Hz, 1H), 7.12 (s, 1H), 6.45 (s, 1H), 4.34 (s, 2H), 4.06 (s, 3H), 3.43 (d, J = 9.6 Hz, 1H), 3.27 (s, 4H), 2.99-2.86 (m, 1H), 2.38 (dq, J = 15.6, 8.8, 7.8 Hz, 1H), 2.22-2.00 (m, 2H), 1.89 (dddd, J = 33.0, 11.4, 9.0, 4.7 Hz, 3H), 1.22 (d, J = 6.9 Hz, 6H). | 423.30 | |
| rel-N-(5-((1R,3S)-3- ((4-cyanopyridazin-3- yl)oxy)cyclopentyl)- 1H-pyrazol-3-yl)-3- (methoxymethyl)-1- methyl-1H-pyrazole- 5-carboxamide | 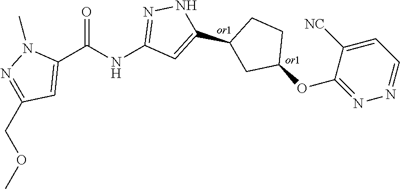 [SMILES: COCc1cc(C(=O)Nc2cc([C@H]3CC[C@@H](Oc4nnccc4C#N)C3)[nH]n2)n(C)n1] | ¹H NMR (400 MHz, DMSO-d₆) 12.29 (s, 1H), 10.71 (s, 1H), 9.16 (d, J = 4.8 Hz, 1H), 8.21 (d, J = 4.8 Hz, 1H), 7.12 (s, 1H), 6.45 (s, 1H), 5.74 (s, 1H), 4.34 (s, 2H), 4.05 (s, 3H), 3.27 (s, 3H), 3.21 (d, J = 8.9 Hz, 1H), 2.78 (dt, J = 14.5, 7.4 Hz, 1H), 2.17 (s, 2H), 1.89 (d, J = 9.9 Hz, 3H). | 423.15 | |
| rel-N-(5-((1R,3S)-3- ((4-cyanopyridazin-3- yl)oxy)cyclopentyl)- 1H-pyrazol-3-yl)-3- (methoxymethyl)-1- methyl-1H-pyrazole- 5-carboxamide | 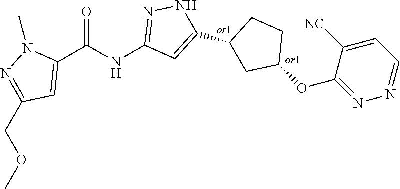 [SMILES: COCc1cc(C(=O)Nc2cc([C@@H]3CC[C@H](Oc4nnccc4C#N)C3)[nH]n2)n(C)n1] | ¹H NMR (400 MHz, DMSO-d₆) 12.30 (s, 1H), 10.71 (s, 1H), 9.16 (d, J = 4.7 Hz, 1H), 8.21 (d, J = 4.8 Hz, 1H), 7.12 (s, 1H), 6.45 (s, 1H), 5.74 (s, 1H), 4.34 (s, 2H), 4.05 (s, 3H), 3.27 (s, 4H), 2.78 (dd, J = 13.8, 7.3 Hz, 1H), 2.17 (s, 2H), 2.03 (s, 1H), 1.90 (s, 2H) | 423.10 | |
| rel-3- (methoxymethyl)-1- methyl-N-(5- ((1R,3S)-3-((6- (trifluoromethyl) pyridazin-3- yl)oxy)cyclopentyl)- 1H-pyrazol-3-yl)-1H- pyrazole-5- carboxamide | 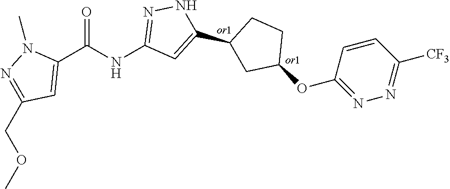 [SMILES: COCc1cc(C(=O)Nc2cc([C@H]3CC[C@@H](Oc4ccc(C(F)(F)F)nn4)C3)[nH]n2)n(C)n1] | ¹H NMR (400 MHz, DMSO-d₆) 12.28 (s, 1H), 10.72 (s, 1H), 8.13 (d, J = 9.3 Hz, 1H), 7.47 (d, J = 9.3 Hz, 1H), 7.12 (s, 1H), 6.46 (s, 1H), 5.71 (s, 1H), 4.34 (s, 2H), 4.05 (s, 3H), 3.27 (s, 4H), 2.75 (dt, J = 14.2, 7.2 Hz, 1H), 2.22-2.11 (m, 2H), 2.02 (d, J = 9.1 Hz, 1H), 1.87 (s, 2H). | 466.30 | |
| rel-3- (methoxymethyl)-1- methyl-N-(5- ((1R,3S)-3-((6- (trifluoromethyl) pyridazin-3- yl)oxy)cyclopentyl)- 1H-pyrazol-3-yl)-1H- pyrazole-5- carboxamide | 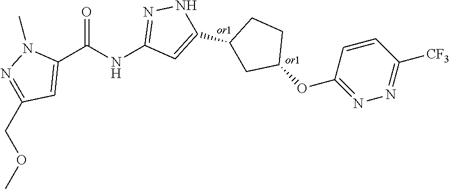 [SMILES: COCc1cc(C(=O)Nc2cc([C@@H]3CC[C@H](Oc4ccc(C(F)(F)F)nn4)C3)[nH]n2)n(C)n1] | ¹H NMR (400 MHz, DMSO-d₆) 12.28 (s, 1H), 10.72 (s, 1H), 8.13 (d, J = 9.3 Hz, 1H), 7.47 (d, J = 9.2 Hz, 1H), 7.12 (s, 1H), 6.45 (s, 1H), 5.71 (d, J = 6.2 Hz, 1H), 4.34 (s, 2H), 4.05 (s, 3H), 3.27 (s, 3H), 3.27-3.18 (m, 1H), 2.75 (dt, J = 14.4, 7.8 Hz, 1H), 2.17 (dt, J = 13.8, 7.1 Hz, 2H), 2.02 (d, J = 9.4 Hz, 1H), 1.87 (s, 2H). | 466.30 | |
| rel-N-(5-((1R,3S)-3- ((6- isopropylpyridazin-3- yl)oxy)cyclopentyl)- 1H-pyrazol-3-yl)-3- (methoxymethyl)-1- methyl-1H-pyrazole- 5-carboxamide | 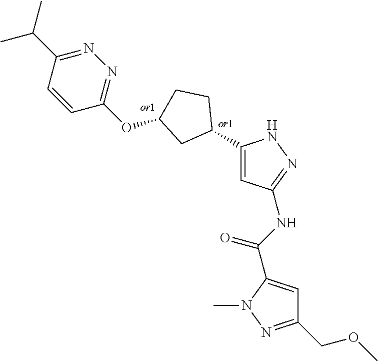 [SMILES: COCc1cc(C(=O)Nc2cc([C@H]3CC[C@@H](Oc4ccc(C(C)C)nn4)C3)[nH]n2)n(C)n1] | ¹H NMR (400 MHz, DMSO-d₆) δ 12.26 (s, 1H), 10.72 (s, 1H), 7.55 (d, J = 9.1 Hz, 1H), 7.14- 7.08 (m, 2H), 6.44 (s, 1H), 5.56 (d, J = 5.2 Hz, 1H), 4.34 (s, 2H), 4.05 (s, 3H), 3.27 (s, 3H), 3.15 (dt, J = 13.8, 7.0 Hz, 2H), 2.70 (dt, J = 14.4, 7.5 Hz, 1H), 2.11 (dq, J = 13.4, 7.1 Hz, 2H), 1.94 (dd, J = 12.7, 4.9 Hz, 1H), 1.81 (ddd, J = 18.7, 8.8, 3.8 Hz, 2H), 1.26 (d, J = 7.0 Hz, 6H). | 440.20 | |
| rel-N-(5-((1R,3S)-3- ((6- isopropylpyridazin-3- yl)oxy)cyclopentyl)- 1H-pyrazol-3-yl)-3- (methoxymethyl)-1- methyl-1H-pyrazole- 5-carboxamide | 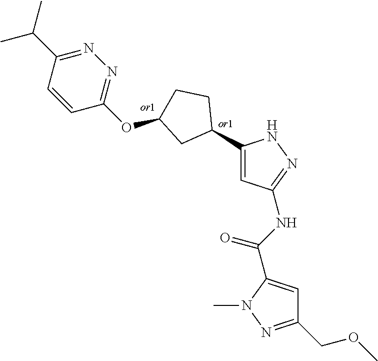 [SMILES: COCc1cc(C(=O)Nc2cc([C@@H]3CC[C@H](Oc4ccc(C(C)C)nn4)C3)[nH]n2)n(C)n1] | ¹H NMR (400 MHz, DMSO-d₆) δ 12.26 (s, 1H), 10.72 (s, 1H), 7.55 (d, J = 9.1 Hz, 1H), 7.14- 7.08 (m, 2H), 6.44 (s, 1H), 5.56 (s, 1H), 4.34 (s, 2H), 4.05 (s, 3H), 3.27 (s, 3H), 3.15 (p, J = 7.0 Hz, 2H), 2.71 (dd, J = 14.3, 7.5 Hz, 1H), 2.11 (dq, J = 13.0, 7.0 Hz, 2H), 1.94 (t, J = 10.5 Hz, 1H), 1.90-1.74 (m, 2H), 1.26 (d, J = 6.9 Hz, 6H). | 440.20 | |
| rel-N-(5-((1R,3S)-3- ((2-isopropylpyridin- 3- yl)oxy)cyclopentyl)- 1H-pyrazol-3-yl)-3- (methoxymethyl)-1- methyl-1H-pyrazole- 5-carboxamide | 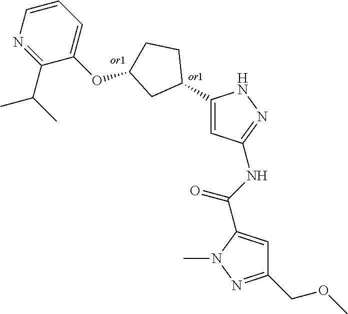 [SMILES: COCc1cc(C(=O)Nc2cc([C@H]3CC[C@@H](Oc4cccnc4C(C)C)C3)[nH]n2)n(C)n1] | ¹H NMR (400 MHz, DMSO-d₆) 12.26 (s, 1H), 10.74 (s, 1H), 8.05 (dd, J = 4.7, 1.4 Hz, 1H), 7.31 (dd, J = 8.4, 1.4 Hz, 1H), 7.19-7.09 (m, 2H), 6.44 (s, 1H), 4.99-4.93 (m, 1H), 4.34 (s, 2H), 4.05 (s, 3H), 3.31 (s, 1H), 3.27 (s, 3H), 3.19 (q, J = 8.3 Hz, 1H), 2.88 (q, J = 7.3 Hz, 1H), 2.68-2.58 (m, 1H), 2.16-1.99 (m, 2H), 1.95-1.76 (m, 2H), 1.19-1.10 (m, 6H) | 439.25 | |
| rel-N-(5-((1R,3S)-3- ((2-isopropylpyridin- 3- yl)oxy)cyclopentyl)- 1H-pyrazol-3-yl)-3- (methoxymethyl)-1- methyl-1H-pyrazole- 5-carboxamide | 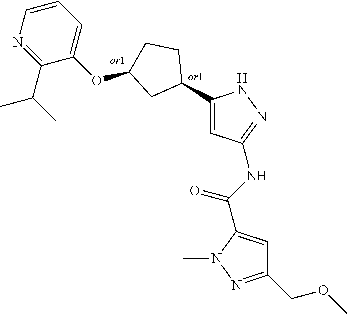 [SMILES: COCc1cc(C(=O)Nc2cc([C@@H]3CC[C@H](Oc4cccnc4C(C)C)C3)[nH]n2)n(C)n1] | ¹H NMR (400 MHz, DMSO-d₆) 12.26 (s, 1H), 10.74 (s, 1H), 8.05 (dd, J = 4.7, 1.3 Hz, 1H), 7.31 (dd, J = 8.3, 1.5 Hz, 1H), 7.19-7.09 (m, 2H), 6.45 (s, 1H), 4.96 (d, J = 4.9 Hz, 1H), 4.34 (s, 2H), 4.05 (s, 3H), 3.38 (p, J = 6.9 Hz, 1H), 3.27 (s, 3H), 3.20 (dd, J = 9.3, 7.1 Hz, 1H), 2.84 (q, J = 7.2 Hz, 1H), 2.63 (ddd, J = 14.5, 8.7, 6.5 Hz, 1H), 2.16-1.99 (m, 2H), 1.95-1.76 (m, 2H), 1.14 (d, J = 6.8 Hz, 6H) | 439.25 | |
| rel-N-(5-((1R,3S)-3- ((4- cyclopropylpyridazin- 3- yl)oxy)cyclopentyl)- 1H-pyrazol-3-yl)-3- (methoxymethyl)-1- methyl-1H-pyrazole- 5-carboxamide | 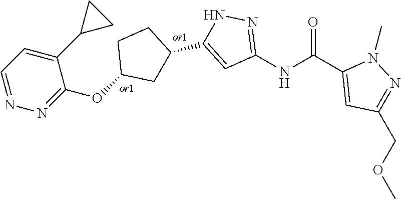 [SMILES: COCc1cc(C(=O)Nc2cc([C@H]3CC[C@@H](Oc4nnccc4C4CC4)C3)[nH]n2)n(C)n1] | ¹H NMR (400 MHz, DMSO-d₆) 12.27 (s, 1H), 10.73 (s, 1H), 8.65 (d, J = 4.8 Hz, 1H), 7.14- 7.05 (m, 2H), 6.48 (s, 1H), 5.66 (s, 1H), 4.34 (s, 2H), 4.05 (s, 3H), 3.30 (d, J = 24.4 Hz, 4H), 2.67 (dt, J = 14.7, 7.7 Hz, 1H), 2.17-1.77 (m, 6H), 1.11-1.02 (m, 2H), 0.86-0.77 (m, 2H). | 438.15 | |
| rel-N-(5-((1R,3S)-3- ((4- cyclopropylpyridazin- 3- yl)oxy)cyclopentyl)- 1H-pyrazol-3-yl)-3- (methoxymethyl)-1- methyl-1H-pyrazole- 5-carboxamide | 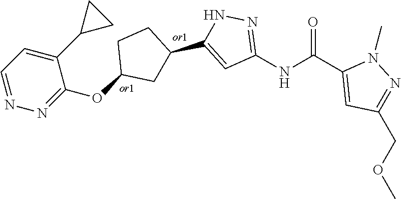 [SMILES: COCc1cc(C(=O)Nc2cc([C@@H]3CC[C@H](Oc4nnccc4C4CC4)C3)[nH]n2)n(C)n1] | ¹H NMR (400 MHz, DMSO-d₆) 12.27 (s, 1H), 10.73 (s, 1H), 8.65 (d, J = 4.8 Hz, 1H), 7.14- 7.06 (m, 2H), 6.49 (s, 1H), 5.66 (d, J = 3.6 Hz, 1H), 4.34 (s, 2H), 4.05 (s, 3H), 3.30 (d, J = 23.0 Hz, 4H), 2.71-2.62 (m, 1H), 2.19-1.75 (m, 6H), 1.11-1.02 (m, 2H), 0.86-0.77 (m, 2H). | 438.15 | |
| rel-3- (methoxymethyl)-1- methyl-N-(5- ((1R,3S)-3-((4- phenylpyridazin-3- yl)oxy)cyclopentyl)- 1H-pyrazol-3-yl)-1H- pyrazole-5- carboxamide | 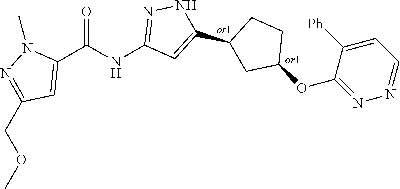 [SMILES: COCc1cc(C(=O)Nc2cc([C@H]3CC[C@@H](Oc4nnccc4-c4ccccc4)C3)[nH]n2)n(C)n1] | ¹H NMR (400 MHz, DMSO-d₆) 12.27 (s, 1H), 10.74 (s, 1H), 8.94 (d, J = 4.8 Hz, 1H), 7.71- 7.63 (m, 3H), 7.54-7.45 (m, 2H), 7.48-7.39 (m, 1H), 7.12 (s, 1H), 6.43 (s, 1H), 5.70 (dt, J = 6.4, 3.1 Hz, 1H), 4.34 (s, 2H), 4.06 (s, 3H), 3.27 (s, 3H), 3.21 (q, J = 9.1, 8.4 Hz, 1H), 2.72 (dt, J = 14.3, 7.2 Hz, 1H), 2.22-2.06 (m, 2H), 1.97 (d, J = 11.0 Hz, 1H), 1.96-1.77 (m, 2H). | 474.15 | |
| rel-3- (methoxymethyl)-1- methyl-N-(5- ((1R,3S)-3-((4- phenylpyridazin-3- yl)oxy)cyclopentyl)- 1H-pyrazol-3-yl)-1H- pyrazole-5- carboxamide | 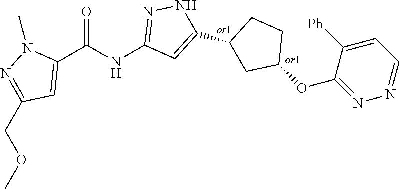 [SMILES: COCc1cc(C(=O)Nc2cc([C@@H]3CC[C@H](Oc4nnccc4-c4ccccc4)C3)[nH]n2)n(C)n1] | ¹H NMR (400 MHz, DMSO-d₆) 12.27 (s, 1H), 10.73 (s, 1H), 8.94 (d, J = 4.7 Hz, 1H), 7.71- 7.63 (m, 3H), 7.54-7.45 (m, 2H), 7.48-7.39 (m, 1H), 7.12 (s, 1H), 6.43 (s, 1H), 5.70 (q, J = 6.5, 5.5 Hz, 1H), 4.34 (s, 2H), 4.06 (s, 3H), 3.27 (s, 3H), 3.21 (q, J = 9.0, 8.1 Hz, 1H), 2.72 (dt, J = 14.3, 7.4 Hz, 1H), 2.13 (tq, J = 13.4, 6.4 Hz, 2H), 2.01-1.77 (m, 3H). | 474.20 | |
| rel-N-(5-((1R,3S)-3- ((2,4- dimethylpyridin-3- yl)oxy)cyclopentyl)- 1H-pyrazol-3-yl)-3- (methoxymethyl)-1- methyl-1H-pyrazole- 5-carboxamide | 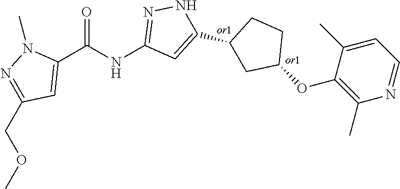 [SMILES: COCc1cc(C(=O)Nc2cc([C@@H]3CC[C@H](Oc4c(C)ccnc4C)C3)[nH]n2)n(C)n1] | ¹H NMR (400 MHz, Chloroform-d) 9.82 (s, 1H), 8.15 (d, J = 4.9 Hz, 1H), 7.02 (d, J = 4.9 Hz, 1H), 6.86 (s, 1H), 6.76 (s, 1H), 4.59-4.51 (m, 1H), 4.52 (s, 2H), 4.19 (s, 3H), 3.48 (s, 3H), 3.21 (p, J = 8.3 Hz, 1H), 2.55 (s, 3H), 2.56-2.46 (m, 1H), 2.32 (s, 3H), 2.28-1.99 (m, 4H), 1.95- 1.82 (m, 1H). | 425.20 | −24.56 |
| rel-N-(5-((1R,3S)-3- ((2,4- dimethylpyridin-3- yl)oxy)cyclopentyl)- 1H-pyrazol-3-yl)-3- (methoxymethyl)-1- methyl-1H-pyrazole- 5-carboxamide | 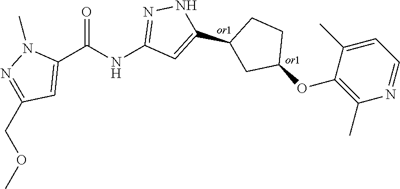 [SMILES: COCc1cc(C(=O)Nc2cc([C@H]3CC[C@@H](Oc4c(C)ccnc4C)C3)[nH]n2)n(C)n1] | ¹H NMR (400 MHz, Chloroform-d) 10.04 (s, 1H), 8.14 (d, J = 4.9 Hz, 1H), 6.98 (d, J = 4.9 Hz, 1H), 6.88 (s, 1H), 6.77 (s, 1H), 4.52 (s, 3H), 4.57-4.47 (m, 1H), 4.18 (s, 3H), 3.48 (s, 3H), 3.18 (p, J = 8.4 Hz, 1H), 2.53 (s, 3H), 2.58-2.44 (m, 1H), 2.30 (s, 3H), 2.24-1.99 (m, 4H), 1.95- 1.82 (m, 1H). | 425.20 | 45.58 |
| rel-3- (methoxymethyl)-1- methyl-N-(5- ((1R,3S)-3-((4- (trifluoromethyl) pyridazin-3- yl)oxy)cyclopentyl)- 1H-pyrazol-3-yl)-1H- pyrazole-5- carboxamide | 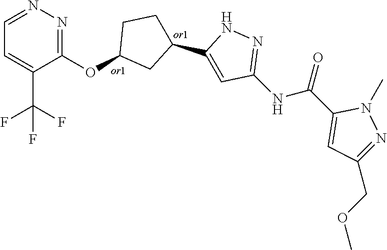 [SMILES: COCc1cc(C(=O)Nc2cc([C@@H]3CC[C@H](Oc4nnccc4C(F)(F)F)C3)[nH]n2)n(C)n1] | ¹H NMR (400 MHz, DMSO-d₆) 12.28 (s, 1H), 10.72 (s, 1H), 9.17 (d, J = 4.8 Hz, 1H), 8.03 (d, J = 4.8 Hz, 1H), 7.11 (s, 1H), 6.42 (s, 1H), 5.79 (s, 1H), 4.34 (s, 2H), 4.05 (s, 3H), 3.20 (d, J = 8.1 Hz, 1H), 2.77 (dt, J = 14.6, 7.6 Hz, 2H), 2.15 (p, J = 6.6 Hz, 2H), 1.99 (t, J = 10.0 Hz, 1H), 1.86 (dd, J = 15.0, 7.9 Hz, 2H). | 466.25 | |
| rel-3- (methoxymethyl)-1- methyl-N-(5- ((1R,3S)-3-((4- (trifluoromethyl) pyridazin-3- yl)oxy)cyclopentyl)- 1H-pyrazol-3-yl)-1H- pyrazole-5- carboxamide | 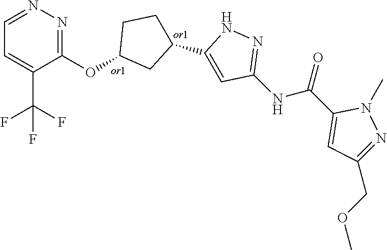 [SMILES: COCc1cc(C(=O)Nc2cc([C@H]3CC[C@@H](Oc4nnccc4C(F)(F)F)C3)[nH]n2)n(C)n1] | ¹H NMR (400 MHz, DMSO-d₆) 12.28 (s, 1H), 10.72 (s, 1H), 9.17 (dd, J = 4.8, 0.8 Hz, 1H), 8.03 (dd, J = 4.8, 0.8 Hz, 1H), 7.11 (s, 1H), 6.42 (s, 1H), 5.82-5.76 (m, 1H), 4.34 (s, 2H), 4.05 (s, 3H), 3.27 (s, 3H), 3.20 (d, J = 8.6 Hz, 1H), 2.77 (dt, J = 14.7, 7.5 Hz, 1H), 2.14 (dq, J = 12.9, 6.7 Hz, 2H), 2.04-1.94 (m, 1H), 1.91-1.79 (m, 2H). | 466.25 | |
| rel-3- (methoxymethyl)-1- methyl-N-(5- ((1R,3S)-3-((3- methylpyrazin-2- yl)oxy)cyclopentyl)- 1H-pyrazol-3-yl)-1H- pyrazole-5- carboxamide | 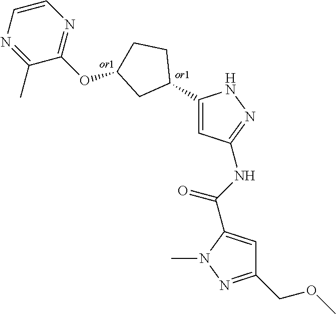 [SMILES: COCc1cc(C(=O)Nc2cc([C@H]3CC[C@@H](Oc4nccnc4C)C3)[nH]n2)n(C)n1] | ¹H NMR (400 MHz, DMSO-d₆) 12.32 (s, 1H), 10.77 (s, 1H), 8.02 (s, 2H), 7.12 (s, 1H), 6.45 (s, 1H), 5.44 (d, J = 4.0 Hz, 1H), 4.34 (s, 2H), 4.05 (s, 2H), 3.36 (s, 3H), 3.21 (t, J = 8.1 Hz, 1H), 2.62 (dt, J = 14.4, 7.5 Hz, 1H), 2.37 (s, 3H), 2.20-2.03 (m, 2H), 1.93-1.77 (m, 3H). | 412.15 | −23 |
| rel-3- (methoxymethyl)-1- methyl-N-(5- ((1R,3S)-3-((3- methylpyrazin-2- yl)oxy)cyclopentyl)- 1H-pyrazol-3-yl)-1H- pyrazole-5- carboxamide | 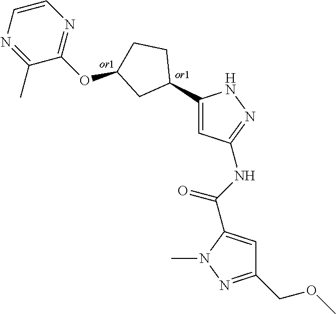 [SMILES: COCc1cc(C(=O)Nc2cc([C@@H]3CC[C@H](Oc4nccnc4C)C3)[nH]n2)n(C)n1] | ¹H NMR (400 MHz, DMSO-d₆) 12.26 (s, 1H), 10.77 (s, 1H), 8.02 (s, 2H), 7.12 (s, 1H), 6.45 (s, 1H), 5.49-5.38 (m, 1H), 4.34 (s, 2H), 4.05 (s, 3H), 3.40 (d, J = 30.8 Hz, 3H), 3.20 (q, J = 8.1 Hz, 1H), 2.62 (dt, J = 14.7, 8.2 Hz, 1H), 2.37 (d, J = 2.5 Hz, 3H), 2.20-2.03 (m, 2H), 1.95- 1.79 (m, 3H). | 412.15 | 26 |
| rel-3- (methoxymethyl)-1- methyl-N-(5- ((1R,3S)-3-((4- methylpyrimidin-5- yl)oxy)cyclopentyl)- 1H-pyrazol-3-yl)-1H- pyrazole-5- carboxamide | 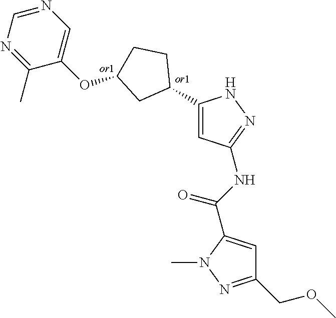 [SMILES: COCc1cc(C(=O)Nc2cc([C@H]3CC[C@@H](Oc4cncnc4C)C3)[nH]n2)n(C)n1] | ¹H NMR (400 MHz) 12.26 (s, 1H), 10.74 (s, 1H), 8.62 (s, 1H), 8.42 (s, 1H), 7.12 (s, 1H), 6.45 (s, 1H), 5.13(m, 1H), 4.34 (s, 2H), 4.05 (s, 3H), 3.27 (s, 3H), 3.21 (dd, J = 9.1, 6.8 Hz, 1H), 2.65 (ddd, J = 14.4, 8.5, 6.4 Hz, 1H), 2.35 (s, 3H), 2.16 (m, 2H), 1.99 (m, 3H). | 412.30 | −5 |
| rel-3- (methoxymethyl)-1- methyl-N-(5- ((1R,3S)-3-((4- methylpyrimidin-5- yl)oxy)cyclopentyl)- 1H-pyrazol-3-yl)-1H- pyrazole-5- carboxamide | 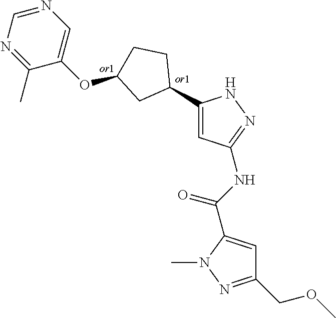 [SMILES: COCc1cc(C(=O)Nc2cc([C@@H]3CC[C@H](Oc4cncnc4C)C3)[nH]n2)n(C)n1] | ¹H NMR (400 MHz) 12.27 (s, 1H), 10.74 (s, 1H), 8.62 (s, 1H), 8.41 (s, 1H), 7.12 (s, 1H), 6.45 (s, 1H), 5.09 (dd, J = 6.6, 3.6 Hz, 1H), 4.34 (s, 2H), 4.05 (s, 3H), 3.27 (s, 3H), 3.20 (q, J = 8.2 Hz, 1H), 2.65 (ddd, J = 14.5, 8.5, 6.4 Hz, 1H), 2.35 (s, 3H), 2.10 (ddt, J = 13.2, 8.7, 4.2 Hz, 2H), 1.87 (dddd, J = 21.6, 17.2, 12.9, 9.6 Hz, 3H). | 412.30 | 6 |
| rel-N-(5-((1R,3S)-3- ((5- isopropylpyridazin-3- yl)oxy)cyclopentyl)- 1H-pyrazol-3-yl)-3- (methoxymethyl)-1- methyl-1H-pyrazole- 5-carboxamide | 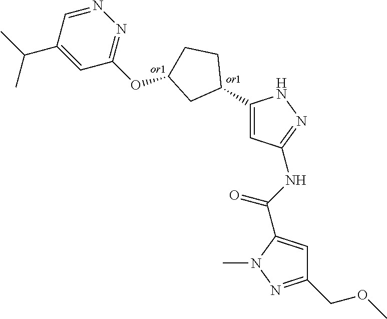 [SMILES: COCc1cc(C(=O)Nc2cc([C@H]3CC[C@@H](Oc4cc(C(C)C)cnn4)C3)[nH]n2)n(C)n1] | ¹H NMR (400 MHz, DMSO-d₆) 12.27 (s, 1H), 10.74 (s, 1H), 8.84 (d, J = 1.8 Hz, 1H), 7.12 (s, 1H), 7.00 (dd, J = 1.8, 0.8 Hz, 1H), 6.45 (s, 1H), 5.60 (d, J = 5.5 Hz, 1H), 4.34 (s, 2H), 4.05 (s, 3H), 3.27 (s, 3H), 3.19 (q, J = 8.4 Hz, 1H), 2.90 (h, J = 6.9 Hz, 1H), 2.74-2.63 (m, 1H), 2.10 (dt, J = 16.4, 6.8 Hz, 2H), 1.94 (d, J = 12.5 Hz, 1H), 1.91-1.75 (m, 2H), 1.21 (dd, J = 6.9, 0.8 Hz, 6H). | 440.15 | 30 |
| rel-N-(5-((1R,3S)-3- ((5- isopropylpyridazin-3- yl)oxy)cyclopentyl)- 1H-pyrazol-3-yl)-3- (methoxymethyl)-1- methyl-1H-pyrazole- 5-carboxamide | 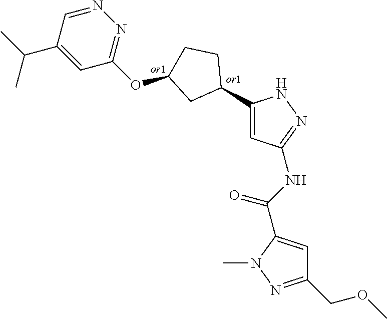 [SMILES: COCc1cc(C(=O)Nc2cc([C@@H]3CC[C@H](Oc4cc(C(C)C)cnn4)C3)[nH]n2)n(C)n1] | ¹H NMR (400 MHz, DMSO-d₆) 12.27 (s, 1H), 10.74 (s, 1H), 8.84 (d, J = 1.9 Hz, 1H), 7.12 (s, 1H), 7.00 (dd, J = 1.8, 0.7 Hz, 1H), 6.45 (s, 1H), 5.60 (d, J = 5.2 Hz, 1H), 4.34 (s, 2H), 4.05 (s, 3H), 3.27 (s, 3H), 3.19 (q, J = 8.3 Hz, 1H), 2.91 (hept, J = 7.0 Hz, 1H), 2.69 (dt, J = 14.4, 7.6 Hz, 1H), 2.18-2.04 (m, 2H), 1.96 (t, J = 10.9 Hz, 1H), 1.83 (dddd, J = 18.2, 13.7, 9.2, 4.3 Hz, 2H), 1.21 (dd, J = 6.9, 0.8 Hz, 6H). | 440.15 | −20 |
| rel-N-(5-((1R,3S)-3- ((3-isopropylpyrazin- 2- yl)oxy)cyclopentyl)- 1H-pyrazol-3-yl)-3- (methoxymethyl)-1- methyl-1H-pyrazole- 5-carboxamide | 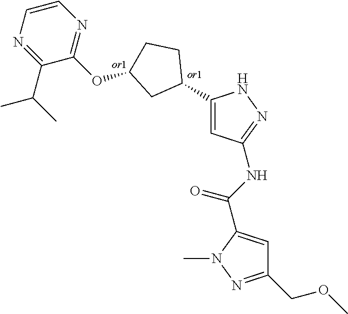 [SMILES: COCc1cc(C(=O)Nc2cc([C@H]3CC[C@@H](Oc4nccnc4C(C)C)C3)[nH]n2)n(C)n1] | ¹H NMR (400 MHz, DMSO-d₆) 12.25 (s, 1H), 10.73 (s, 1H), 8.08 (d, J = 2.8 Hz, 1H), 8.02 (d, J = 2.8 Hz, 1H), 7.12 (s, 1H), 6.47 (s, 1H), 5.48 (dt, J = 6.6, 3.4 Hz, 1H), 4.34 (s, 2H), 4.05 (s, 3H), 3.32-3.22 (m, 1H), 3.27 (s, 3H), 3.21 (d, J = 8.1 Hz, 1H), 2.68-2.57 (m, 1H), 2.10 (s, 2H), 1.99-1.80 (m, 3H), 1.18 (dd, J = 6.9, 1.2 Hz, 6H). | 440.15 | −24 |
| rel-N-(5-((1R,3S)-3- ((3-isopropylpyrazin- 2- yl)oxy)cyclopentyl)- 1H-pyrazol-3-yl)-3- (methoxymethyl)-1- methyl-1H-pyrazole- 5-carboxamide | 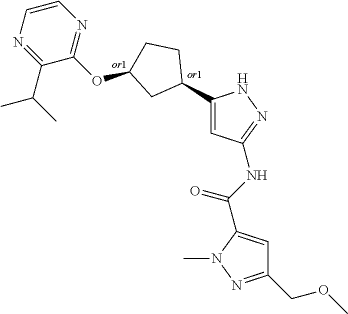 [SMILES: COCc1cc(C(=O)Nc2cc([C@@H]3CC[C@H](Oc4nccnc4C(C)C)C3)[nH]n2)n(C)n1] | ¹H NMR (400 MHz, DMSO-d₆) 12.26 (s, 1H), 10.73 (s, 1H), 8.08 (d, J = 2.8 Hz, 1H), 8.02 (d, J = 2.8 Hz, 1H), 7.12 (s, 1H), 6.48 (s, 1H), 5.51- 5.45 (m, 1H), 4.34 (s, 2H), 4.05 (s, 3H), 3.27 (s, 3H), 3.32-3.16 (m, 2H), 2.62 (dt, J = 14.6, 7.6 Hz, 1H), 2.10 (s, 1H), 1.99-1.80 (m, 3H), 1.18 (dd, J = 6.9, 1.3 Hz, 6H). | 440.20 | 13 |
| rel-N-(5-((1R,3S)-3- ((3-isopropylpyridin- 2- yl)oxy)cyclopentyl)- 1H-pyrazol-3-yl)-3- (methoxymethyl)-1- methyl-1H-pyrazole- 5-carboxamide | 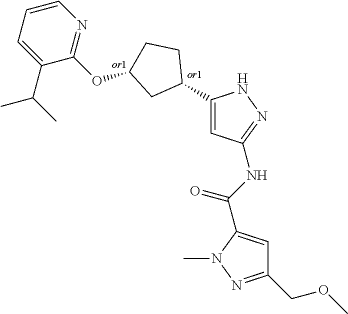 [SMILES: COCc1cc(C(=O)Nc2cc([C@H]3CC[C@@H](Oc4ncccc4C(C)C)C3)[nH]n2)n(C)n1] | ¹H NMR (400 MHz, DMSO-d₆) 12.25 (s, 1H), 10.72 (s, 1H), 7.98 (dd, J = 5.0, 1.9 Hz, 1H), 7.53 (dd, J = 7.3, 2.0 Hz, 1H), 7.12 (s, 1H), 6.91 (dd, J = 7.3, 4.9 Hz, 1H), 6.46 (s, 1H), 5.49 (dd, J = 8.2, 5.2 Hz, 1H), 4.34 (s, 2H), 4.05 (s, 3H), 3.24-3.14 (m, 1H), 3.09 (p, J = 6.8 Hz, 1H), 2.62 (dt, J = 14.7, 7.2 Hz, 1H), 2.10-2.00 (m, 2H), 1.95-1.75 (m, 3H), 1.16 (d, J = 6.9 Hz, 6H). | 439.25 | |
| rel-N-(5-((1R,3S)-3- ((3-isopropylpyridin- 2- yl)oxy)cyclopentyl)- 1H-pyrazol-3-yl)-3- (methoxymethyl)-1- methyl-1H-pyrazole- 5-carboxamide | 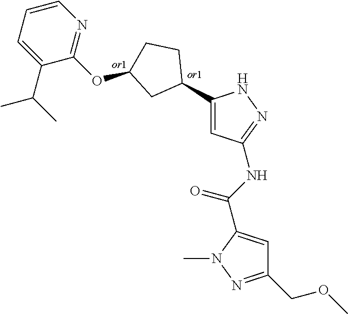 [SMILES: COCc1cc(C(=O)Nc2cc([C@@H]3CC[C@H](Oc4ncccc4C(C)C)C3)[nH]n2)n(C)n1] | ¹H NMR (400 MHz, DMSO-d₆) 12.25 (s, 1H), 10.72 (s, 1H), 7.98 (dd, J = 4.9, 1.9 Hz, 1H), 7.54 (dd, J = 7.5, 1.8 Hz, 1H), 7.12 (s, 1H), 6.91 (dd, J = 7.3, 5.0 Hz, 1H), 6.46 (s, 1H), 5.49 (dd, J = 8.6, 4.9 Hz, 1H), 4.34 (s, 2H), 4.05 (s, 3H), 3.25-3.14 (m, 1H), 3.09 (p, J = 6.9 Hz, 1H), 2.62 (dt, J = 14.6, 7.7 Hz, 1H), 2.17-1.99 (m, 2H), 1.95-1.76 (m, 3H), 1.16 (d, J = 6.9 Hz, 6H). | 439.25 | |
| rel-N-(5-((1R,3S)-3- ((2- (difluoromethyl) pyridin-3- yl)oxy)cyclopentyl)- 1H-pyrazol-3-yl)-3- (methoxymethyl)-1- methyl-1H-pyrazole- 5-carboxamide | 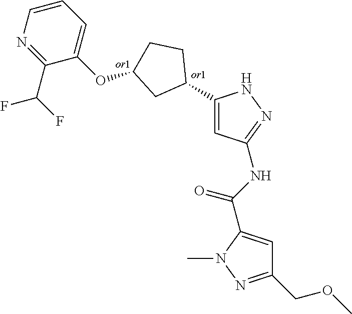 [SMILES: COCc1cc(C(=O)Nc2cc([C@H]3CC[C@@H](Oc4cccnc4C(F)F)C3)[nH]n2)n(C)n1] | ¹H NMR (400 MHz, DMSO-d₆) 12.27 (s, 1H), 10.73 (s, 1H), 8.23 (dd, J = 4.5, 1.1 Hz, 1H), 7.66 (d, J = 8.5 Hz, 1H), 7.54 (dd, J = 8.5, 4.5 Hz, 1H), 7.14 (d, J = 14.5 Hz, 1H), 7.02 (s, 1H), 6.46 (s, 1H), 5.08 (dd, J = 6.9, 4.0 Hz, 1H), 4.34 (s, 2H), 4.05 (s, 3H), 3.27 (s, 3H), 3.18 (q, J = 8.4 Hz, 1H), 2.66 (dt, J = 14.4, 7.8 Hz, 1H), 2.12-2.06 (m, 1H), 2.10-2.00 (m, 1H), 1.98-1.78 (m, 3H). | 447.15 | 3 |
| rel-N-(5-((1R,3S)-3- ((2- (difluoromethyl) pyridin-3- yl)oxy)cyclopentyl)- 1H-pyrazol-3-yl)-3- (methoxymethyl)-1- methyl-1H-pyrazole- 5-carboxamidee | 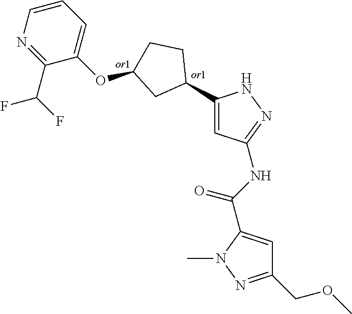 [SMILES: COCc1cc(C(=O)Nc2cc([C@@H]3CC[C@H](Oc4cccnc4C(F)F)C3)[nH]n2)n(C)n1] | ¹H NMR (400 MHz, DMSO-d₆) 12.27 (s, 1H), 10.73 (s, 1H), 8.23 (dd, J = 4.5, 1.2 Hz, 1H), 7.66 (d, J = 8.5 Hz, 1H), 7.54 (dd, J = 8.6, 4.6 Hz, 1H), 7.14 (d, J = 14.6 Hz, 1H), 7.02 (s, 1H), 6.45 (s, 1H), 5.08 (dt, J = 9.2, 4.8 Hz, 1H), 4.34 (s, 2H), 4.05 (s, 3H), 3.27 (s, 3H), 3.19 (t, J = 8.1 Hz, 1H), 2.71-2.60 (m, 1H), 2.13-2.00 (m, 2H), 1.97-1.74 (m, 3H). | 447.20 | −8 |
| rel-N-(5-((1R,3S)-3- ((4-ethylpyridazin-3- yl)oxy)cyclopentyl)- 1H-pyrazol-3-yl)-3- (methoxymethyl)-1- methyl-1H-pyrazole- 5-carboxamide | 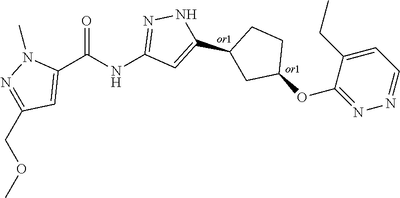 [SMILES: CCc1ccnnc1O[C@@H]1CC[C@H](c2cc(NC(=O)c3cc(COC)nn3C)n[nH]2)C1] | ¹H NMR (400 MHz, DMSO-d₆) 12.26 (s, 1H), 10.73 (s, 1H), 8.74 (d, J = 4.6 Hz, 1H), 7.42 (dd, J = 4.7, 1.1 Hz, 1H), 7.12 (s, 1H), 6.46 (s, 1H), 5.66 (dq, J = 6.5, 3.2 Hz, 1H), 4.34 (s, 2H), 4.05 (s, 3H), 3.27 (s, 3H), 3.27-3.19 (m, 1H), 2.67 (ddd, J = 14.5, 8.7, 6.5 Hz, 1H), 2.61- 2.51 (m, 2H), 2.19-2.10 (m, 1H), 2.11 (s, 1H), 1.98-1.82 (m, 3H), 1.15 (t, J = 7.5 Hz, 3H). | 426.15 | |
| rel-N-(5-((1R,3S)-3- ((4-ethylpyridazin-3- yl)oxy)cyclopentyl)- 1H-pyrazol-3-yl)-3- (methoxymethyl)-1- methyl-1H-pyrazole- 5-carboxamide | 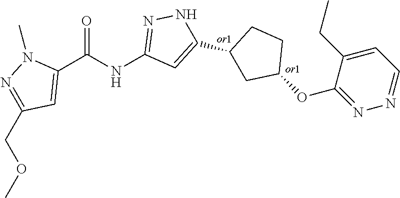 [SMILES: CCc1ccnnc1O[C@H]1CC[C@@H](c2cc(NC(=O)c3cc(COC)nn3C)n[nH]2)C1] | ¹H NMR (400 MHz, DMSO-d₆) 12.26 (s, 1H), 10.73 (s, 1H), 8.74 (d, J = 4.7 Hz, 1H), 7.42 (d, J = 4.6 Hz, 1H), 7.11 (s, 1H), 6.45 (s, 1H), 5.65 (td, J = 6.4, 3.3 Hz, 1H), 4.34 (s, 2H), 4.05 (s, 3H), 3.27 (s, 3H), 3.27-3.19 (m, 1H), 2.67 (ddd, J = 14.6, 8.7, 6.5 Hz, 1H), 2.55 (t, J = 7.5 Hz, 2H), 2.13 (qd, J = 9.6, 8.8, 6.2 Hz, 2H), 1.96 (td, J = 10.2, 9.5, 3.9 Hz, 1H), 1.88 (ddt, J = 13.6, 7.4, 4.8 Hz, 2H), 1.13 (t, J = 7.5 Hz, 3H). | 426.20 | |
| rel-3- (methoxymethyl)-1- methyl-N-(3- ((1R,3S)-3- (pyrido[3,4- d]pyridazin-1- yloxy)cyclopentyl)- 1H-pyrazol-5-yl)-1H- pyrazole-5- carboxamide | 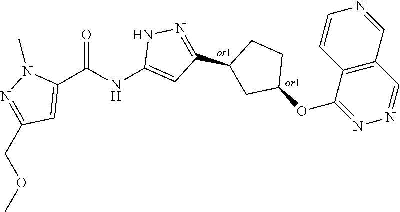 [SMILES: COCc1cc(C(=O)Nc2cc([C@H]3CC[C@@H](Oc4nncc5cnccc45)C3)n[nH]2)n(C)n1] | ¹H NMR (400 MHz, DMSO-d₆) 12.29 (s, 1H), 10.73 (s, 1H), 9.55-9.47 (m, 2H), 9.03 (d, J = 5.5 Hz, 1H), 8.01 (d, J = 5.3 Hz, 1H), 7.12 (s, 1H), 6.51 (s, 1H), 5.83 (d, J = 4.7 Hz, 1H), 4.34 (s, 2H), 4.05 (s, 3H), 3.27 (s, 4H), 2.74 (dt, J = 14.6, 7.5 Hz, 1H), 2.22-2.16 (m, 2H), 2.14 (dd, J = 17.5, 7.1 Hz, 1H), 2.05 (ddd, J = 13.1, 8.1, 3.9 Hz, 1H), 2.00-1.90 (m, 1H). | 449.10 | |
| rel-3- (methoxymethyl)-1- methyl-N-(3- ((1R,3S)-3- (pyrido[3,4- d]pyridazin-1- yloxy)cyclopentyl)- 1H-pyrazol-5-yl)-1H- pyrazole-5- carboxamide | 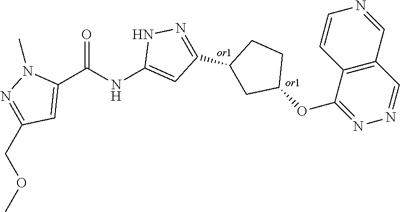 [SMILES: COCc1cc(C(=O)Nc2cc([C@@H]3CC[C@H](Oc4nncc5cnccc45)C3)n[nH]2)n(C)n1] | ¹H NMR (400 MHz, DMSO-d₆) 12.29 (s, 1H), 10.73 (s, 1H), 9.55-9.46 (m, 2H), 9.03 (dd, J = 5.6, 2.3 Hz, 1H), 8.01 (d, J = 5.3 Hz, 1H), 7.12 (s, 1H), 6.51 (s, 1H), 5.83 (s, 1H), 4.34 (d, J = 2.2 Hz, 2H), 4.05 (d, J = 2.1 Hz, 3H), 3.27 (d, J = 2.2 Hz, 4H), 2.73 (dd, J = 14.1, 7.3 Hz, 1H), 2.18 (s, 2H), 2.12 (d, J = 9.1 Hz, 1H), 2.08-2.01 (m, 1H), 1.97 (t, J = 8.2 Hz, 1H). | 449.10 | |
| rel-N-(5-((1R,3S)-3- ((4- cyclobutylpyridazin- 3- yl)oxy)cyclopentyl)- 1H-pyrazol-3-yl)-3- (methoxymethyl)-1- methyl-1H-pyrazole- 5-carboxamide | 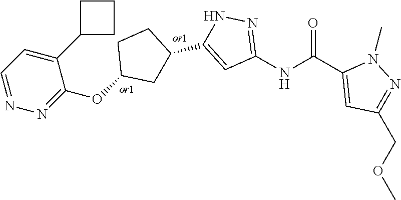 [SMILES: COCc1cc(C(=O)Nc2cc([C@H]3CC[C@@H](Oc4nnccc4C4CCC4)C3)[nH]n2)n(C)n1] | ¹H NMR (400 MHz, DMSO-d₆) 12.26 (s, 1H), 10.76 (s, 1H), 8.76 (d, J = 4.7 Hz, 1H), 7.41 (dd, J = 4.7, 1.2 Hz, 1H), 7.12 (s, 1H), 6.47 (s, 1H), 5.88-5.45 (m, 1H), 4.34 (s, 2H), 4.06 (s, 3H), 3.55 (t, J = 8.5 Hz, 1H), 3.27 (s, 4H), 2.65 (ddd, J = 14.7, 8.8, 6.6 Hz, 1H), 2.32-2.19 (m, 2H), 2.18-1.79 (m, 8H), 1.79-1.68 (m, 1H). | 452.30 | |
| rel-N-(5-((1R,3S)-3- ((4- cyclobutylpyridazin- 3- yl)oxy)cyclopentyl)- 1H-pyrazol-3-yl)-3- (methoxymethyl)-1- methyl-1H-pyrazole- 5-carboxamide | 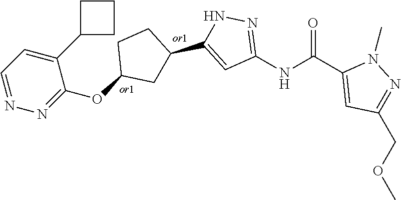 [SMILES: COCc1cc(C(=O)Nc2cc([C@@H]3CC[C@H](Oc4nnccc4C4CCC4)C3)[nH]n2)n(C)n1] | ¹H NMR (400 MHz, DMSO-d₆) 12.26 (s, 1H), 10.75 (s, 1H), 8.76 (d, J = 4.7 Hz, 1H), 7.41 (dd, J = 4.7, 1.2 Hz, 1H), 7.12 (s, 1H), 6.47 (s, 1H), 5.94-5.55 (m, 1H), 4.34 (s, 2H), 4.06 (s, 3H), 3.61-3.48 (m, 1H), 3.27 (s, 4H), 2.69-2.58 (m, 1H), 2.33-2.20 (m, 2H), 2.18-1.98 (m, 5H), 1.98-1.69 (m, 4H). | 452.30 | |
| ((1s,3s)-3-(2-(3- (methoxymethyl)-1- methyl-1H-pyrazole- 5- carboxamido)thiazol- 5- yl)cyclobutyl)methyl isopropylcarbamate | 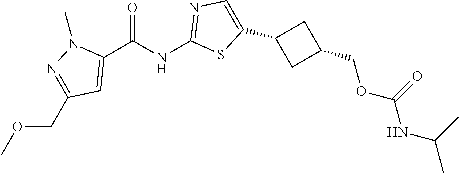 [SMILES: COCc1cc(C(=O)Nc2ncc([C@H]3C[C@@H](COC(=O)NC(C)C)C3)s2)n(C)n1] | ¹H NMR (400 MHz, DMSO-d₆) 7.20 (s, 1H), 7.10 (s, 1H), 7.03 (d, J = 7.8 Hz, 1H), 6.71 (s, 1H), 4.35 (s, 2H), 4.10 (s, 3H), 3.93 (d, J = 6.0 Hz, 2H), 3.58 (s, 2H), 3.27 (s, 3H), 2.48-2.30 (m, 3H), 1.87 (d, J = 10.1 Hz, 2H), 1.05 (d, J = 6.6 Hz, 6H). | 422.15 | |
| rel-(3R,5R)-5-(3-((2- (methoxymethyl) pyrazolo[1,5-a]pyrazin-4- yl)amino)-1H- pyrazol-5- yl)tetrahydrofuran-3- yl(2S,5S)-2,5- dimethylpyrrolidine- 1-carboxylate | 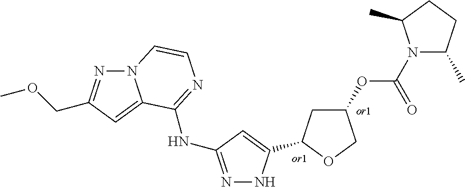 | 1H NMR (400 MHz, DMSO-d6) 12.38 (s, 1H), 10.04 (s, 1H), 8.04 (s, 1H), 7.40-7.25 (m, 2H), 6.82 (s, 1H), 5.25 (d, J = 5.9 Hz, 1H), 5.00 (s, 1H), 4.57 (s, 2H), 3.91-3.79 (m, 4H), 3.30 (s, 3H), 2.67 (dt, J = 14.7, 7.8 Hz, 1H), 2.13 (ddd, J = 13.8, 5.8, 2.3 Hz, 1H), 2.02 (s, 2H), 1.42 (d, J = 6.9 Hz, 2H), 1.06 (d, J = 6.3 Hz, 3H), 0.98 (d, J = 6.1 Hz, 3H). | 456.15 | |
| rel-(3R,5R)-5-(3-((2- (methoxymethyl) pyrazolo[1,5-a]pyrazin-4- yl)amino)-1H- pyrazol-5- yl)tetrahydrofuran-3- yl(2S,5S)-2,5- dimethylpyrrolidine- 1-carboxylate | 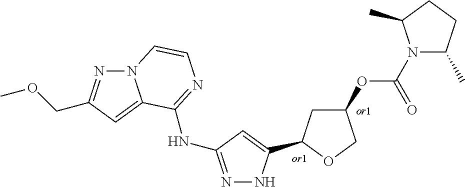 | 1H NMR (400 MHz, DMSO-d6) 12.39 (s, 1H), 10.05 (s, 1H), 8.04 (s, 1H), 7.44-7.23 (m, 2H), 6.82 (s, 1H), 5.22 (td, J = 4.6, 2.2 Hz, 1H), 4.98 (s, 1H), 4.57 (s, 2H), 3.95-3.80 (m, 4H), 3.34 (s, 3H), 2.73- 2.65 (m, 1H), 2.12-2.02 (m, 3H), 1.44 (d, J = 5.8 Hz, 2H), 1.08 (d, J = 6.3 Hz, 3H), 1.00 (d, J = 6.3 Hz, 3H). | 456.15 | |
| ((1s,3s)-3-(3-(3- (ethoxymethyl)-1- methyl-1H-pyrazole- 5-carboxamido)-1H- pyrazol-5- yl)cyclobutyl)methyl isopropylcarbamate | 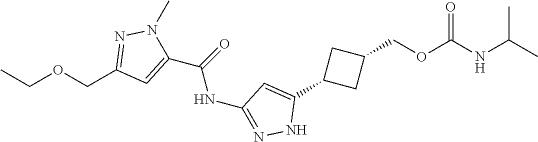 [SMILES: CCOCc1cc(C(=O)Nc2cc([C@H]3C[C@@H](COC(=O)NC(C)C)C3)[nH]n2)n(C)n1] | 1H NMR (400 MHz, DMSO-d6) 12.24 (s, 1H), 10.74 (s, 1H), 7.13 (s, 1H), 7.03 (d, J = 7.8 Hz, 1H), 6.46 (s, 1H), 4.38 (s, 2H), 4.06 (s, 2H), 3.94 (d, J = 6.4 Hz, 2H), 3.59 (dd, J = 12.5, 5.8 Hz, 1H), 3.48 (q, J = 7.0 Hz, 2H), 2.42-2.33 (m, 2H), 1.96- 1.86 (m, 3H), 1.13 (t, J = 7.0 Hz, 4H), 1.05 (d, J = 6.6 Hz, 6H). | 419.25 | |
| ((1r,3r)-3-(3-(3- (ethoxymethyl)-1- methyl-1H-pyrazole- 5-carboxamido)-1H- pyrazol-5- yl)cyclobutyl)methyl isopropylcarbamate | 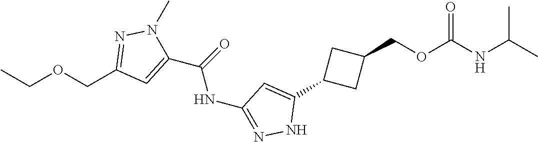 [SMILES: CCOCc1cc(C(=O)Nc2cc([C@H]3C[C@H](COC(=O)NC(C)C)C3)[nH]n2)n(C)n1] | 1H NMR (400 MHz, DMSO-d6) 12.25 (s, 1H), 10.75 (s, 1H), 7.13 (s, 1H), 7.07 (d, J = 7.8 Hz, 1H), 6.50 (s, 1H), 4.38 (s, 2H), 4.06 (d, J = 6.7 Hz, 5H), 3.59 (ddd, J = 15.6, 12.7, 7.3 Hz, 2H), 3.48 (d, J = 7.0 Hz, 1H), 2.28-2.13 (m, 3H), 1.13 (t, J = 7.0 Hz, 3H), 1.05 (d, J = 6.5 Hz, 6H). | 419.20 | |
| rel-(3R,5R)-5-(3-((2- (methoxymethyl) pyrazolo[1,5-a]pyrazin-4- yl)amino)-1H- pyrazol-5- yl)tetrahydrofuran-3- yl tert-butylcarbamate | 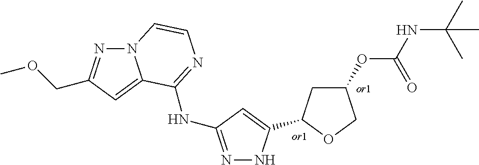 [SMILES: COCc1cc2c(Nc3cc([C@@H]4C[C@H](OC(=O)NC(C)(C)C)CO4)[nH]n3)nccn2n1] | 1H NMR (400 MHz, Methanol-d4) 7.96 (d, J = 4.4 Hz, 1H), 7.40 (m, 1H), 7.07 (s, 1H), 6.39 (s, 1H), 5.23 (s, 1H), 4.99 (s, 1H), 4.67 (s, 2H), 4.06 (d, J = 10.2 Hz, 1H), 3.96 (dd, J = 10.3, 4.8 Hz, 1H), 3.46 (s, 3H), 2.70 (m, 1H), 2.09 (d, J = 70.8 Hz, 1H), 1.30 (s, 9H). | 430.20 | 9 |
| rel-(3R,5R)-5-(3-((2- (methoxymethyl) pyrazolo[1,5-a] pyrazin-4-yl)amino)-1H- pyrazol-5- yl)tetrahydrofuran-3- yl tert-butylcarbamate | 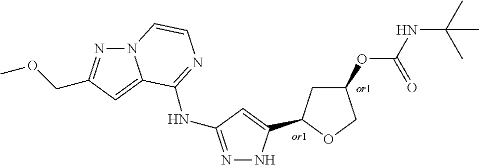 [SMILES: COCc1cc2c(Nc3cc([C@H]4C[C@@H](OC(=O)NC(C)(C)C)CO4)[nH]n3)nccn2n1] | 1H NMR (400 MHz, Methanol-d4) 7.95 (d, J = 4.4 Hz, 1H), 7.45 (m, 1H), 7.07 (s, 1H), 6.39 (s, 1H), 5.25 (s, 1H), 5.00 (s, 1H), 4.67 (s, 2H), 4.06 (d, J = 10.2 Hz, 1H), 3.98 (dd, J = 10.3, 4.8 Hz, 1H), 3.46 (s, 3H), 2.76 (m, 1H), 2.12 (d, J = 70.8 Hz, 1H), 1.31 (s, 9H). | 430.20 | −9 |
| rel-(3R,5R)-5-(3-((2- (methoxymethyl) pyrazolo[1,5-a] pyrazin-4-yl)amino)-1H- pyrazol-5- yl)tetrahydrofuran-3- yl (1- methylcyclopropyl) carbamate | 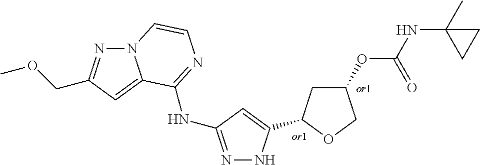 [SMILES: COCc1cc2c(Nc3cc([C@@H]4C[C@H](OC(=O)NC5(C)CC5)CO4)[nH]n3)nccn2n1] | 1H NMR (400 MHz, Methanol-d4) 7.96 (d, J = 4.8 Hz, 1H), 7.44 (d, J = 4.9 Hz, 1H), 7.07 (s, 1H), 6.38 (s, 1H), 5.26 (s, 1H), 5.00 (d, J = 7.7 Hz, 1H), 4.67 (s, 2H), 4.06 (d, J = 10.3 Hz, 2H), 3.46 (s, 3H), 2.75 (dt, J = 14.4, 7.5 Hz, 1H), 2.17 (dd, J = 13.5, 7.6 Hz, 1H), 1.33 (m, 3H), 0.60 (t, J = 3.3 Hz, 4H). | 428.20 | 5 |
| rel-(3R,5R)-5-(3-((2- (methoxymethyl) pyrazolo[1,5-a] pyrazin-4-yl)amino)-1H- pyrazol-5- yl)tetrahydrofuran-3- yl (1- methylcyclopropyl) carbamate | 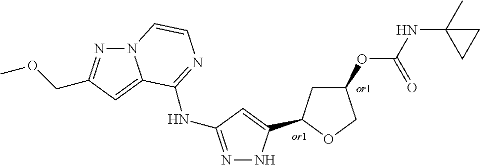 [SMILES: COCc1cc2c(Nc3cc([C@H]4C[C@@H](OC(=O)NC5(C)CC5)CO4)[nH]n3)nccn2n1] | 1H NMR (400 MHz, Methanol-d4) 7.96 (d, J = 4.8 Hz, 1H), 7.45 (d, J = 4.9 Hz, 1H), 7.07 (s, 1H), 6.38 (s, 1H), 5.26 (s, 1H), 4.99 (d, J = 7.7 Hz, 1H), 4.67 (s, 2H), 4.06 (d, J = 10.3 Hz, 2H), 3.46 (s, 3H), 2.74 (dt, J = 14.4, 7.5 Hz, 1H), 2.16 (dd, J = 13.5, 7.6 Hz, 1H), 1.32 (m, 3H), 0.60 (t, J = 3.3 Hz, 4H). | 428.20 | −6 |
| (3R,5R)-5-(3-((2- (methoxymethyl) pyrazolo[1,5-a] pyrazin-4-yl)amino)-1H- pyrazol-5- yl)tetrahydrofuran-3- yl tert-butylcarbamate | 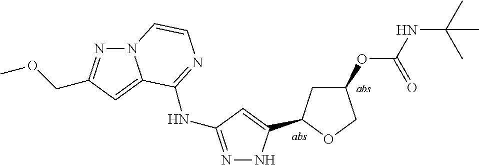 [SMILES: COCc1cc2c(Nc3cc([C@H]4C[C@@H](OC(=O)NC(C)(C)C)CO4)[nH]n3)nccn2n1] | 1H NMR (400 MHz, DMSO-d6) 12.37 (s, 1H), 10.06 (s, 1H), 8.06 (s, 1H), 7.40 (d, J = 4.8 Hz, 1H), 7.29 (s, 1H), 6.99 (s, 1H), 6.81 (s, 1H), 5.17 (s, 1H), 4.85 (s, 1H), 4.58 (s, 2H), 3.86 (d, J = 7.3 Hz, 2H), 3.28 (s, 3H), 2.72 (s, 1H), 1.96 (s, 1H), 1.22 (s, 9H). | 430.20 | 11 |
| (3R,5R)-5-(3-((2- (methoxymethyl) pyrazolo[1,5-a]pyrazin-4- yl)amino)-1H- pyrazol-5- yl)tetrahydrofuran-3- yl (1- methylcyclopropyl) carbamate | 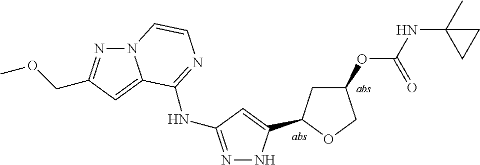 [SMILES: COCc1cc2c(Nc3cc([C@H]4C[C@@H](OC(=O)NC5(C)CC5)CO4)[nH]n3)nccn2n1] | 1H NMR (400 MHz, DMSO-d6) 12.38 (s, 1H), 10.05 (s, 1H), 8.06 (s, 1H), 7.55 (s, 1H), 7.40 (d, J = 4.8 Hz, 1H), 7.29 (s, 1H), 6.99 (s, 1H), 6.80 (s, 1H), 5.18 (s, 1H), 4.85 (s, 1H), 4.58 (s, 2H), 3.86 (s, 2H), 3.34 (s, 3H), 1.96 (s, 1H), 1.25 (s, 3H), 0.61 (s, 2H), 0.49 (q, J = 4.6 Hz, 2H). | 428.20 | 9 |
| ((1S,3R)-3-(3-((2- (methoxymethyl) pyrazolo[1,5-a]pyrazin-4- yl)amino)-1H- pyrazol-5- yl)cyclobutyl)methyl (2S,5S)-2,5- dimethylpyrrolidine- 1-carboxylate | 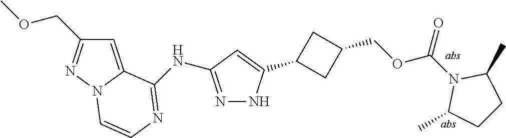 | 1H NMR (400 MHz, DMSO-d6) 12.12 (s, 1H), 9.97 (s, 1H), 8.03 (d, J = 4.7 Hz, 1H), 7.38 (d, J = 4.7 Hz, 1H), 7.29 (s, 1H), 6.67 (s, 1H), 4.57 (s, 2H), 4.04 (dd, J = 11.0, 5.8 Hz, 1H), 3.99- 3.81 (m, 3H), 3.33 (s, 4H), 2.43-2.33 (m, 3H), 2.09 (q, J = 3.5 Hz, 2H), 1.98 (qd, J = 9.7, 4.2 Hz, 2H), 1.48 (dt, J = 9.7, 5.7 Hz, 2H), 1.11 (dd, J = 12.8, 6.3 Hz, 6H). | 454.15 | |
| rel-(3R,5R)-5-(3-((2- (methoxymethyl) pyrazolo[1,5-a]pyrazin-4- yl)amino)-1H- pyrazol-5- yl)tetrahydrofuran-3- yl ((R*)-4,4,4- trifluorobutan-2- yl)carbamate | 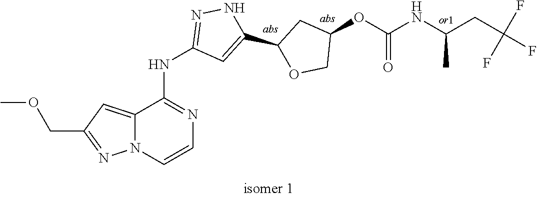 [SMILES: COCc1cc2c(Nc3cc([C@H]4C[C@@H](OC(=O)N[C@H](C)CC(F)(F)F)CO4)[nH]n3)nccn2n1] | 1H NMR (400 MHz, DMSO-d6) 12.42 (s, 1H), 10.09 (s, 1H), 8.06 (s, 1H), 7.45-7.33 (m, 2H), 7.28 (s, 1H), 6.78 (s, 1H), 5.19-4.84 (m, 2H), 4.57 (s, 2H), 3.93-3.79 (m, 3H), 3.33 (s, 3H), 2.73 (s, 1H), 2.45-2.33 (m, 2H), 1.97 (s, 1H), 1.15 (t, J = 7.0 Hz, 3H). | 484.10 | 8 |
| rel-(3R,5R)-5-(3-((2- (methoxymethyl) pyrazolo[1,5-a]pyrazin-4- yl)amino)-1H- pyrazol-5- yl)tetrahydrofuran-3- yl ((R*)-4,4,4- trifluorobutan-2- yl)carbamate | 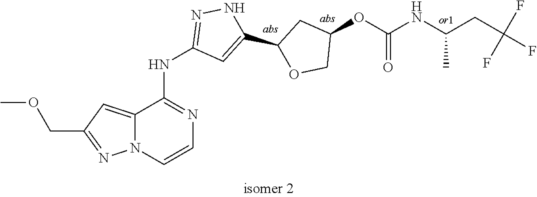 [SMILES: COCc1cc2c(Nc3cc([C@H]4C[C@@H](OC(=O)N[C@@H](C)CC(F)(F)F)CO4)[nH]n3)nccn2n1] | 1H NMR (400 MHz, DMSO-d6) 12.43 (s, 1H), 10.09 (s, 1H), 8.06 (s, 1H), 7.43-7.34 (m, 2H), 7.28 (s, 1H), 6.78 (s, 1H), 5.19-4.84 (m, 2H), 4.58 (s, 2H), 3.91-3.80 (m, 3H), 3.33 (s, 3H), 2.73 (s, 1H), 2.40 (dd, J = 13.6, 8.0 Hz, 2H), 1.99 (s, 1H), 1.16-1.11 (m, 3H) | 484.15 | 10 |
| (3R,5R)-5-(3-((2- (methoxymethyl) pyrazolo[1,5-a]pyrazin-4- yl)amino)-1H- pyrazol-5- yl)tetrahydrofuran-3- yl (1- (trifluoromethyl) cyclopropyl)carbamate | 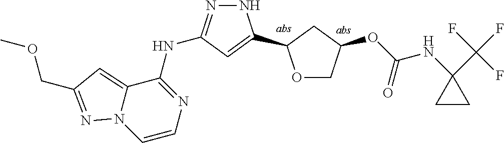 [SMILES: COCc1cc2c(Nc3cc([C@H]4C[C@@H](OC(=O)NC5(C(F)(F)F)CC5)CO4)[nH]n3)nccn2n1] | 1H NMR (400 MHz, DMSO-d6) 12.39 (s, 1H), 10.06 (s, 1H), 8.28 (s, 1H), 8.05 (s, 1H), 7.39 (d, J = 4.7 Hz, 1H), 7.28 (s, 1H), 6.82 (d, J = 19.7 Hz, 1H), 5.21 (s, 1H), 4.84 (s, 1H), 4.57 (s, 2H), 3.94-3.83 (m, 2H), 3.33 (s, 3H), 2.73 (s, 1H), 2.03 (d, J = 43.5 Hz, 1H), 1.20 (s, 2H), 1.05 (s, 2H). | 482.20 | 6 |
| (3R,5R)-5-(3-((2- (methoxymethyl) pyrazolo[1,5-a]pyrazin-4- yl)amino)-1H- pyrazol-5- yl)tetrahydrofuran-3- yl (1-(2,2,2- trifluoroethyl)cyclo- propyl)carbamate | 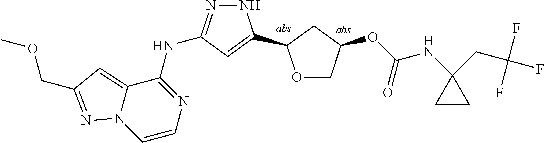 [SMILES: COCc1cc2c(Nc3cc([C@H]4C[C@@H](OC(=O)NC5(CC(F)(F)F)CC5)CO4)[nH]n3)nccn2n1] | 1H NMR (400 MHz, DMSO-d6) 12.38 (s, 1H), 10.04 (s, 1H), 8.05 (s, 1H), 7.80 (s, 1H), 7.45- 7.24 (m, 2H), 6.79 (s, 1H), 5.23 (d, J = 21.0 Hz, 1H), 4.92 (d, J = 71.1 Hz, 1H), 4.57 (s, 2H), 3.94-3.82 (m, 2H), 3.33 (s, 3H), 2.72 (s, 1H), 2.55 (s, 1H), 2.48-2.44 (m, 1H), 1.94 (s, 1H), 0.78-0.61 (m, 4H). | 496.10 | 5 |
| (3R,5R)-5-(3-((2- (methoxymethyl) pyrazolo[1,5-a]pyrazin-4- yl)amino)-1H- pyrazol-5- yl)tetrahydrofuran-3- yl(1,1,1-trifluoro-2- methylpropan-2- yl)carbamate | 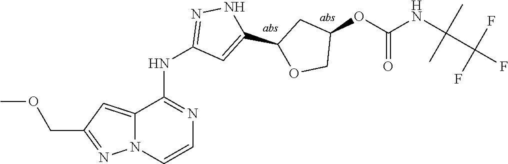 [SMILES: COCc1cc2c(Nc3cc([C@H]4C[C@@H](OC(=O)NC(C)(C)C(F)(F)F)CO4)[nH]n3)nccn2n1] | 1H NMR (400 MHz, DMSO-d6) 12.40 (s, 1H), 10.06 (s, 1H), 8.05 (s, 1H), 7.53 (s, 1H), 7.40 (d, J = 4.7 Hz, 1H), 7.31 (s, 1H), 6.83 (s, 1H), 6.20 (s, 1H), 5.20 (s, 1H), 4.87 (s, 1H), 4.58 (s, 2H), 3.91 (d, J = 10.3 Hz, 1H), 3.86 (d, J = 5.0 Hz, 1H), 3.34 (s, 3H), 2.75 (s, 1H), 1.25 (s, 6H). | 484.20 | 15 |
| (3R,5R)-5-(3-((2- (methoxymethyl) pyrazolo[1,5-a]pyrazin-4- yl)amino)-1H- pyrazol-5- yl)tetrahydrofuran-3- yl(1,1-difluoro-2- methylpropan-2- yl)carbamate | 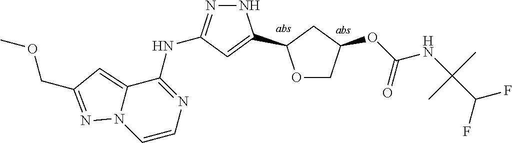 [SMILES: COCc1cc2c(Nc3cc([C@H]4C[C@@H](OC(=O)NC(C)(C)C(F)F)CO4)[nH]n3)nccn2n1] | 1H NMR (400 MHz, DMSO-d6) 12.39 (s, 1H), 10.05 (s, 1H), 8.05 (s, 1H), 7.67 (s, 1H), 7.39 (d, J = 4.8 Hz, 1H), 7.30 (s, 1H), 6.82 (s, 1H), 5.19 (s, 1H), 4.86 (s, 1H), 4.58 (s, 2H), 3.91 (d, J = 10.3 Hz, 1H), 3.85 (dd, J = 11.0, 4.6 Hz, 1H), 3.34 (s, 2H), 2.74 (s, 1H), 1.98 (s, 1H), 1.45 (d, J = 1.9 Hz, 6H). | 466.20 | 15 |
| (3R,5R)-5-(3-((2- (methoxymethyl) pyrazolo[1,5-a]pyrazin-4- yl)amino)-1H- pyrazol-5- yl)tetrahydrofuran-3- yl (1- (difluoromethyl)cyclo- propyl)carbamate | 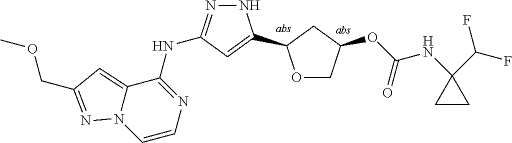 [SMILES: COCc1cc2c(Nc3cc([C@H]4C[C@@H](OC(=O)NC5(C(F)F)CC5)CO4)[nH]n3)nccn2n1] | 1H NMR (400 MHz, DMSO-d6) 12.39 (s, 1H), 10.04 (s, 1H), 8.02 (d, J = 17.2 Hz, 2H), 7.35 (d, J = 34.8 Hz, 2H), 6.81 (s, 1H), 5.90 (t, J = 56.6 Hz, 1H), 5.21 (s, 1H), 4.87 (s, 1H), 4.57 (s, 2H), 4.00-3.80 (m, 2H), 3.34 (s, 3H), 2.73 (s, 1H), 1.97 (s, 1H), 1.00-0.95 (m, 2H), 0.86 (s, 2H). | 464.10 | +7 |
| ((1s,3s)-3-(3-(1- methyl-3- ((trifluoromethoxy) methyl)-1H-pyrazole-5- carboxamido)-1H- pyrazol-5- yl)cyclobutyl)methyl isopropylcarbamate | 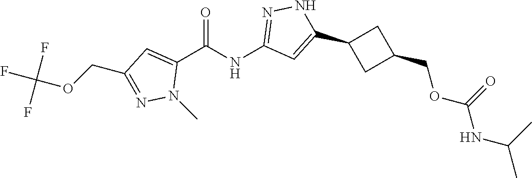 [SMILES: CC(C)NC(=O)OC[C@H]1C[C@@H](c2cc(NC(=O)c3cc(COC(F)(F)F)nn3C)n[nH]2)C1] | 1H NMR (400 MHz, DMSO-d6) 12.25 (s, 1H), 10.84 (s, 1H), 7.29 (s, 1H), 7.01 (d, J = 7.8 Hz, 1H), 6.46 (s, 1H), 5.11 (s, 2H), 4.10 (s, 3H), 3.94 (d, J = 6.4 Hz, 2H), 3.58 (dt, J = 13.4, 6.7 Hz, 1H), 3.38 (d, J = 9.0 Hz, 1H), 2.55 (d, J = 8.4 Hz, 1H), 2.36 (qd, J = 8.1, 2.6 Hz, 2H), 1.90 (q, J = 10.0 Hz, 2H), 1.05 (d, J = 6.5 Hz, 6H). | 459.10 | |
| ((1s,3s)-3-(3-(3- ((difluoromethoxy) methyl)-1-methyl-1H- pyrazole-5- carboxamido)-1H- pyrazol-5- yl)cyclobutyl)methyl isopropylcarbamate | 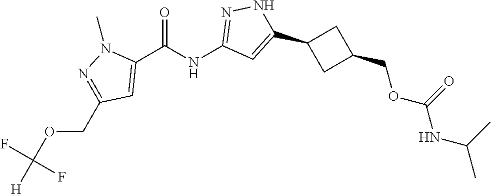 [SMILES: CC(C)NC(=O)OC[C@H]1C[C@@H](c2cc(NC(=O)c3cc(COC(F)F)nn3C)n[nH]2)C1] | 1H NMR (400 MHz, DMSO-d6) 12.31-12.19 (m, 1H), 10.80 (s, 1H), 7.22 (s, 1H), 7.02 (d, J = 7.8 Hz, 1H), 6.77 (t, J = 75.6 Hz, 1H), 6.46 (s, 1H), 4.85 (s, 2H), 4.08 (s, 3H), 3.94 (d, J = 6.4 Hz, 2H), 3.59 (h, J = 6.7 Hz, 1H), 3.41 (s, 1H), 2.54 (d, J = 6.0 Hz, 1H), 2.36 (qd, J = 8.1, 2.6 Hz, 2H), 1.90 (q, J = 10.1, 9.7 Hz, 2H), 1.05 (d, J = 6.5 Hz, 6H). | 441.10 | |
| (3R,5R)-5-(3-((2- (ethoxymethyl)pyrazolo [1,5-a]pyrazin-4- yl)amino)-1H- pyrazol-5- yl)tetrahydrofuran-3- yl isopropylcarbamate | 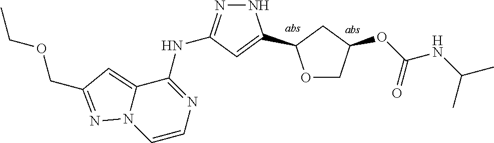 [SMILES: CCOCc1cc2c(Nc3cc([C@H]4C[C@@H](OC(=O)NC(C)C)CO4)[nH]n3)nccn2n1] | 1H NMR (400 MHz, DMSO-d6) 12.37 (s, 1H), 10.02 (s, 1H), 8.03 (s, 1H), 7.38 (s, 1H), 7.31 (s, 1H), 7.14 (d, J = 7.8 Hz, 1H), 6.81 (s, 1H), 5.18 (s, 1H), 4.87 (s, 1H), 4.61 (s, 2H), 3.87 (s, 2H), 3.57 (dq, J = 20.9, 6.8 Hz, 3H), 2.72 (s, 1H), 1.97 (s, 1H), 1.17 (t, J = 7.0 Hz, 3H), 1.08- 1.02 (m, 6H). | 430.20 | +27 |
| (3R,5R)-5-(3-((2- (methoxymethyl) pyrazolo[1,5-a]pyrazin-4- yl)amino)-1H- pyrazol-5- yl)tetrahydrofuran-3- yl ((R)-1- cyclopropylethyl) carbamate | 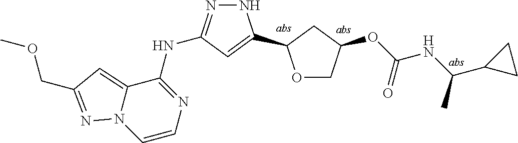 [SMILES: COCc1cc2c(Nc3cc([C@H]4C[C@@H](OC(=O)N[C@H](C)C5CC5)CO4)[nH]n3)nccn2n1] | 1H NMR (400 MHz, DMSO-d6) 12.39 (s, 1H), 10.03 (s, 1H), 8.04 (s, 1H), 7.39 (d, J = 4.8 Hz, 1H), 7.30 (s, 1H), 7.19 (d, J = 8.4 Hz, 1H), 6.81 (s, 1H), 5.18 (s, 1H), 4.86 (s, 1H), 4.57 (s, 2H), 3.87 (s, 2H), 3.33 (s, 3H), 3.00 (q, J = 7.5 Hz, 1H), 2.73 (s, 1H), 1.98 (s, 1H), 1.10 (d, J = 6.7 Hz, 3H), 0.81 (s, 1H), 0.49-0.20 (m, 3H), 0.11 (s, 1H), | 442.20 | +12 |
| ((1s,3s)-3-(3-(3- (isopropoxymethyl)- 1-methyl-1H- pyrazole-5- carboxamido)-1H- pyrazol-5- yl)cyclobutyl)methyl | 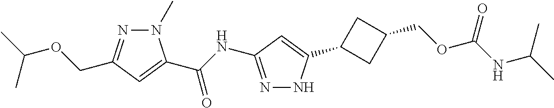 [SMILES: CC(C)NC(=O)OC[C@H]1C[C@@H](c2cc(NC(=O)c3cc(COC(C)C)nn3C)n[nH]2)C1] | 1H NMR (400 MHz, DMSO-d6) 12.23 (s, 1H), 10.72 (s, 1H), 7.23-6.89 (m, 2H), 6.45 (s, 1H), 4.38 (s, 2H), 4.05 (s, 3H), 3.94 (d, J = 6.4 Hz, 2H), 3.62 (ddt, J = 28.8, 13.4, 6.4 Hz, 2H), 3.37 (d, J = 9.0 Hz, 1H), 2.52 (s, 1H), 2.36 (qd, J = 8.2, 2.7 Hz, 2H), 1.94-1.84 (m, 2H), 1.13 (d, J = 6.1 Hz, 6H), 1.05 (d, J = 6.6 Hz, 6H). | 433.30 | |
| isopropylcarbamate | | | | |
| (3R,5R)-5-(3-((2- (methoxymethyl) pyrazolo[1,5-a]pyrazin-4- yl)amino)-1H- pyrazol-5- yl)tetrahydrofuran-3-yl bicyclo[1.1.1]pentan- 1-ylcarbamate | 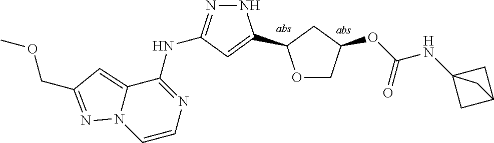 [SMILES: COCc1cc2c(Nc3cc([C@H]4C[C@@H](OC(=O)NC56CC(C5)C6)CO4)[nH]n3)nccn2n1] | 1H NMR (400 MHz, DMSO-d6) 12.39 (s, 1H), 10.04 (s, 1H), 8.00 (d, J = 40.7 Hz, 2H), 7.52- 7.08 (m, 2H), 6.81 (s, 1H), 5.29-5.06 (m, 1H), 4.87 (s, 1H), 4.57 (s, 2H), 3.87 (s, 2H), 3.33 (s, 3H), 2.70 (d, J = 14.8 Hz, 1H), 2.36 (s, 1H), 1.91 (s, 7H). | 440.20 | +9 |
| (3R,5R)-5-(3-((2- (methoxymethyl) pyrazolo[1,5-a]pyrazin-4- yl)amino)-1H- pyrazol-5- yl)tetrahydrofuran-3- yl((S)-1- cyclopropylethyl) carbamate | 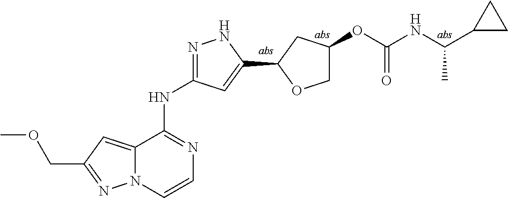 [SMILES: COCc1cc2c(Nc3cc([C@H]4C[C@@H](OC(=O)N[C@@H](C)C5CC5)CO4)[nH]n3)nccn2n1] | 1H NMR (400 MHz, DMSO-d6) 12.39 (s, 1H), 10.04 (s, 1H), 8.04 (s, 1H), 7.40 (d, J = 4.8 Hz, 1H), 7.30 (s, 1H), 7.20 (d, J = 8.5 Hz, 1H), 6.81 (s, 1H), 5.17 (s, 1H), 4.85 (s, 1H), 4.57 (s, 2H), 3.90-3.86 (m, 2H), 3.33 (s, 1H), 3.32 (s, 2H), 2.99 (q, J = 7.5 Hz, 1H), 2.71 (s, 1H), 1.97 (s, 1H), 1.09 (d, J = 6.7 Hz, 3H), 1.00 (s, 1H), 0.33 (s, 2H), 0.290.21 (m, 1H), 0.15-0.08 (m, 1H). | 442.20 | 24 |
| (3R,5R)-5-(3-((2- (methoxymethyl) pyrazolo[1,5-a]pyrazin-4- yl)amino)-1H- pyrazol-5- yl)tetrahydrofuran-3- yl ((R)-sec- butyl)carbamate | 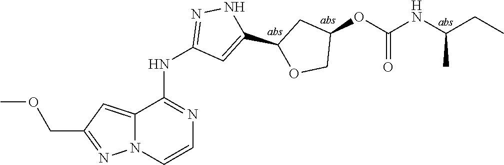 [SMILES: CC[C@@H](C)NC(=O)O[C@H]1CO[C@@H](c2cc(Nc3nccn4nc(COC)cc34)n[nH]2)C1] | 1H NMR (400 MHz, DMSO-d6) 12.45 (s, 1H), 10.32 (s, 1H), 8.08 (d, J = 4.9 Hz, 1H), 7.40 (d, J = 4.9 Hz, 1H), 7.31 (s, 1H), 7.00 (dd, J = 47.6, 8.8 Hz, 1H), 6.65 (s, 1H), 5.18 (s, 1H), 4.86 (t, J = 7.6 Hz, 1H), 4.58 (s, 2H), 3.88 (qd, J = 10.3, 3.6 Hz, 2H), 3.53-3.38 (m, 4H), 2.72 (dt, J = 14.7, 7.7 Hz, 1H), 1.97 (d, J = 10.6 Hz, 1H), 1.36 (dq, J = 14.7, 7.2, 6.6 Hz, 2H), 1.02 (d, J = 6.6 Hz, 3H), 0.80 (t, J = 7.4 Hz, 3H). | 340.25 | 28 |
| (3R,5R)-5-(3-((2- (methoxymethyl) pyrazolo[1,5-a]pyrazin-4- yl)amino)-1H- pyrazol-5- yl)tetrahydrofuran-3- yl carbamate | 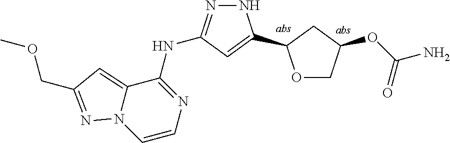 [SMILES: COCc1cc2c(Nc3cc([C@H]4C[C@@H](OC(N)=O)CO4)[nH]n3)nccn2n1] | 1H NMR (400 MHz, DMSO-d6) 12.41 (s, 1H), 10.18 (s, 1H), 8.05 (d, J = 4.8 Hz, 1H), 7.40 (d, J = 4.8 Hz, 1H), 7.27 (s, 1H), 6.66 (s, 3H), 5.14 (q, J = 4.0 Hz, 1H), 4.84 (t, J = 7.7 Hz, 1H), 4.57 (s, 2H), 3.87 (qd, J = 10.1, 3.6 Hz, 2H), 3.33 (s, 3H), 2.70 (dt, J = 14.2, 7.4 Hz, 1H), 2.04-1.93 (m, 1H) | 374.05 | −76 |
| (3R,5R)-5-(3-((2- (isopropoxymethyl) pyrazolo[1,5-a]pyrazin- 4-yl)amino)-1H- pyrazol-5- yl)tetrahydrofuran-3- yl isopropylcarbamate | 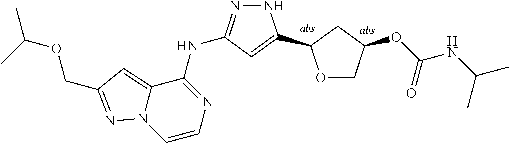 [SMILES: CC(C)NC(=O)O[C@H]1CO[C@@H](c2cc(Nc3nccn4nc(COC(C)C)cc34)n[nH]2)C1] | 1H NMR (400 MHz, DMSO-d6) 12.34(s, 1H), 10.05 (s, 1H), 8.04 (s, 1H), 7.38 (d, J = 4.8 Hz, 1H), 7.27 (s, 1H), 7.14 (d, J = 7.8 Hz, 1H), 7.12(s, 1H), 5.18 (s, 1H), 4.84 (s, 1H), 4.62 (s, 2H), 3.88 (d, J = 13.0 Hz, 2H), 3.72 (q, J = 6.1 Hz, 1H), 3.69(m, 1H), 2.68 (s, 1H), 1.97 (s, 1H), 1.16 (d, J = 6.1 Hz, 6H), 1.07-0.94 (m, 6H). | 444.10 | 11 |
| (3R,5R)-5-(3-((2- ((difluoromethoxy) methyl)pyrazolo[1,5- a]pyrazin-4- yl)amino)-1H- pyrazol-5- yl)tetrahydrofuran-3- yl isopropylcarbamate | 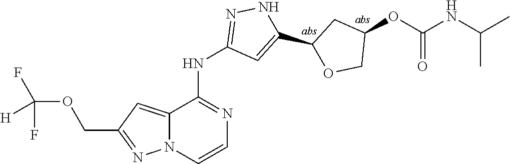 [SMILES: CC(C)NC(=O)O[C@H]1CO[C@@H](c2cc(Nc3nccn4nc(COC(F)F)cc34)n[nH]2)C1] | 1H NMR (400 MHz, DMSO-d6) 12.38 (s, 1H), 10.12 (s, 1H), 8.07 (s, 1H), 7.46-7.38 (m, 2H), 7.13 (d, J = 7.8 Hz, 1H), 6.84 (s, 1H), 6.81 (s, 1H), 5.18 (s, 1H), 5.08 (s, 2H), 4.86 (s, 1H), 3.87 (s, 2H), 3.60 (dt, J = 13.7, 6.8 Hz, 1H), 2.71 (s, 1H), 2.05 (s, 1H), 1.04 (dd, J = 6.7, 2.9 Hz, 6H). | 452.20 | 12 |
| (3R,5R)-5-(3-((2- (morpholinomethyl) pyrazolo[1,5- a]pyrazin-4- yl)amino)-1H- pyrazol-5- yl)tetrahydrofuran-3- yl isopropylcarbamate | 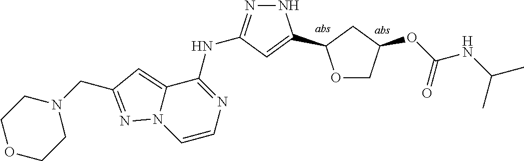 [SMILES: CC(C)NC(=O)O[C@H]1CO[C@@H](c2cc(Nc3nccn4nc(CN5CCOCC5)cc34)n[nH]2)C1] | 1H NMR (400 MHz, DMSO-d6) 12.27 (s, 1H), 10.74 (s, 1H), 8.76 (d, J = 4.7 Hz, 1H), 7.44 (d, J = 4.7 Hz, 1H), 7.12 (s, 1H), 6.41 (s, 1H), 5.55 (s, 1H), 4.34 (s, 2H), 4.06 (s, 3H), 3.27 (s, 3H), 3.11 (h, J = 6.8 Hz, 1H), 2.80 (s, 1H), 2.09 (s, 2H), 1.94-1.71 (m, 6H), 1.23 (d, J = 6.9 Hz, 6H). | 471.20 | 102 |
| (3R,5R)-5-(5-((2- (methoxymethyl) pyrazolo[1,5-a]pyrazin-4- yl)amino)-1H- pyrazol-3- yl)tetrahydrofuran-3- yl cyclobutylcarbamate | 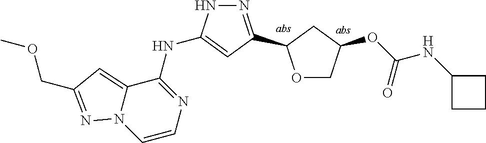 [SMILES: COCc1cc2c(Nc3cc([C@H]4C[C@@H](OC(=O)NC5CCC5)CO4)n[nH]3)nccn2n1] | 1H NMR (400 MHz, DMSO-d6) 12.36 (s, 1H), 10.07 (s, 2H), 8.05 (s, 3H), 7.54 (d, J = 8.1 Hz, 3H), 7.40 (d, J = 4.8 Hz, 4H), 7.27 (s, 2H), 6.77 (s, 1H), 5.17 (s, 3H), 4.84 (s, 2H), 4.57 (s, 7H), 4.01-3.80 (m, 11H), 2.71 (s, 2H), 2.14-2.06 (m, 7H), 1.97 (s, 2H), 1.93-1.83 (m, 6H), 1.58-1.54 (m, 4H). | 428.10 | 12.00 |
| (3R,5R)-5-(5-((2- (methoxymethyl) pyrazolo[1,5-a]pyrazin-4- yl)amino)-1H- pyrazol-3- yl)tetrahydrofuran-3- yl (1- (methoxymethyl)cyclo- propyl)carbamate | 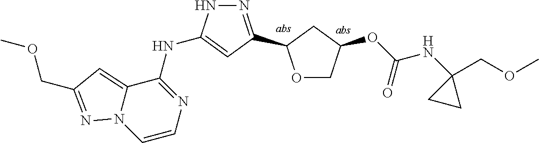 | 1H NMR (400 MHz, DMSO-d6) δ 12.35 (s, 1H), 10.02 (s, 1H), 8.03 (s, 1H), 7.59 (s, 1H), 7.39 (s, 2H), 6.80 (s, 1H), 5.18 (s, 1H), 4.86 (s, 1H), 4.57 (s, 2H), 3.86 (s, 2H), 3.3 (s, 4H),3.2 (s, 4H), 2.72 (d, J = 9.0 Hz, 1H), 1.94 (s, 1H), 0.64 (s, 4H). | 458.20 | 7 |
| (3R,5R)-5-(3-((2- (methoxymethyl) pyrazolo[1,5-a]pyrazin-4- yl)amino)-1H- pyrazol-5- yl)tetrahydrofuran-3- yl ((S)-3- methylbutan-2- yl)carbamate | 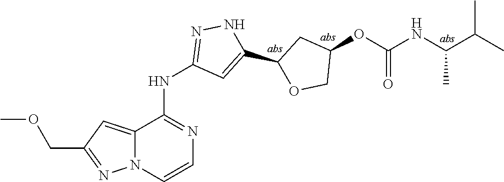 [SMILES: COCc1cc2c(Nc3cc([C@H]4C[C@@H](OC(=O)N[C@@H](C)C(C)C)CO4)[nH]n3)nccn2n1] | 1H NMR (400 MHz, DMSO-d6) 12.36 (s, 1H), 10.06 (s, 1H), 8.05 (s, 1H), 7.39 (d, J = 4.8 Hz, 1H), 7.27 (s, 1H), 7.09 (d, J = 8.7 Hz, 1H), 6.79 (s, 1H), 5.18 (s, 1H), 4.84 (s, 1H), 4.57 (s, 2H), 3.96-3.79 (m, 2H), 3.33 (s, 3H), 3.31 (d, J = 8.2 Hz, 1H), 2.82-2.61 (m, 1H), 1.99 (s, 1H), 1.60 (q, J = 6.7 Hz, 1H), 0.97 (d, J = 6.8 Hz, 3H), 0.82 (dd, J = 6.8, 3.5 Hz, 6H). | 444.15 | +19.33 |
| (3R,5R)-5-(3-((2- (azetidin-1- ylmethyl)pyrazolo[1,5- a]pyrazin-4- yl)amino)-1H- pyrazol-5- yl)tetrahydrofuran-3- yl isopropylcarbamate | 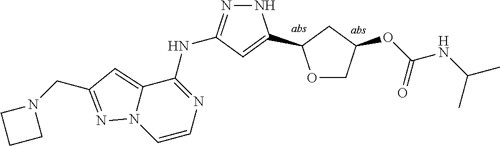 [SMILES: CC(C)NC(=O)O[C@H]1CO[C@@H](c2cc(Nc3nccn4nc(CN5CCC5)cc34)n[nH]2)C1] | 1H NMR (400 MHz, DMSO-d6) 12.35 (s, 1H), 9.94 (s, 1H), 8.00 (s, 1H), 7.35 (s, 1H), 7.23- 6.96 (m, 2H), 6.79 (s, 1H), 5.18 (s, 1H), 4.85 (s, 1H), 3.87 (s, 2H), 3.67 (s, 2H), 3.59 (q, J = 6.8 Hz, 1H), 3.20 (t, J = 6.9 Hz, 4H), 2.71 (s, 1H), 1.99 (p, J = 6.6 Hz, 3H), 1.04 (dd, J = 6.8, 3.0 Hz, 6H). | 441.20 | +15 |
| (3R,5R)-5-(3-((2- (methoxymethyl) pyrazolo[1,5-a]pyrazin-4- yl)amino)-1H- pyrazol-5- yl)tetrahydrofuran-3- yl (1- methylcyclobutyl) carbamate | 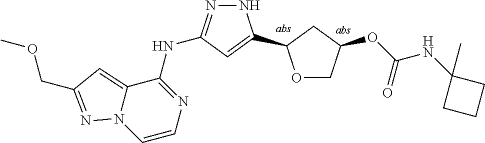 [SMILES: COCc1cc2c(Nc3cc([C@H]4C[C@@H](OC(=O)NC5(C)CCC5)CO4)[nH]n3)nccn2n1] | 1H NMR (400 MHz, DMSO-d6) 12.36 (s, 1H), 10.02 (s, 1H), 8.03 (s, 1H), 7.56-7.05 (m, 3H), 6.81 (s, 1H), 5.17 (s, 1H), 4.87 (s, 1H), 4.57 (s, 2H), 3.87 (s, 2H), 3.33 (s, 3H), 2.89-2.63 (m, 1H), 2.35-2.12 (m, 2H), 1.98 (s, 1H), 1.89-1.77 (m, 2H), 1.76-1.57 (m, 2H), 1.32 (s, 3H). | 442.20 | +13 |
| (3R,5R)-5-(3-((2- (476yrrolidine-1- ylmethyl)pyrazolo[1,5- a]pyrazin-4- yl)amino)-1H- pyrazol-5- yl)tetrahydrofuran-3- yl isopropylcarbamate | 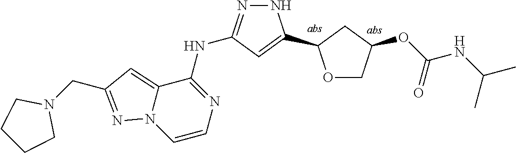 [SMILES: CC(C)NC(=O)O[C@H]1CO[C@@H](c2cc(Nc3nccn4nc(CN5CCCC5)cc34)n[nH]2)C1] | 1H NMR (400 MHz, DMSO-d6) 12.35 (s, 1H), 9.96 (s, 1H), 8.02 (s, 1H), 7.36 (d, J = 4.8 Hz, 1H), 7.23 (s, 1H), 7.14 (d, J = 7.8 Hz, 1H), 6.79 (s, 1H), 5.18 (s, 1H), 4.85 (s, 1H), 3.87 (s, 2H), 3.77 (s, 2H), 3.60 (q, J = 6.7 Hz, 1H), 2.68 (d, J = 2.2 Hz, 1H), 2.55 (s, 4H), 1.98 (s, 1H), 1.72 (s, 4H), 1.05 (dd, J = 6.8, 2.7 Hz, 6H). | 455.25 | 9 |
| (3R,5R)-5-(3-((2- (methoxymethyl) pyrazolo[1,5-a]pyrazin-4- yl)amino)-1H- pyrazol-5- yl)tetrahydrofuran-3- yl (1- (fluoromethyl)cyclo- propyl)carbamate | 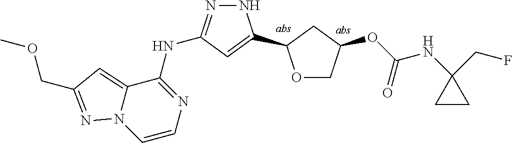 [SMILES: COCc1cc2c(Nc3cc([C@H]4C[C@@H](OC(=O)NC5(CF)CC5)CO4)[nH]n3)nccn2n1] | 1H NMR (400 MHz, DMSO-d6) 8.02 (s, 1H), 7.79 (s, 1H), 7.38 (d, J = 4.8 Hz, 1H), 7.27 (s, 1H), 6.65 (s, 1H), 5.20 (s, 1H), 4.81 (s, 1H), 4.57 (s, 2H), 4.34 (d, J = 49.1 Hz, 2H), 3.94- 3.79 (m, 2H), 3.33 (s, 3H), 2.75-2.66 (m, 1H), 1.98 (s, 1H), 0.77 (d, J = 9.2 Hz, 4H). | 446.20 | 15 |
| (3R,5R)-5-(3-((2- (methoxymethyl) pyrazolo[1,5-a]pyrazin-4- yl)amino)-1H- pyrazol-5- yl)tetrahydrofuran-3- yl ((R*)-2,2- dimethylcyclopropyl) carbamate | 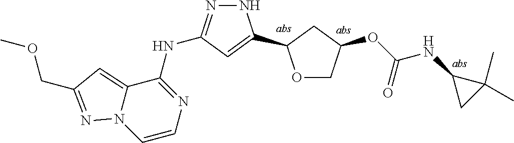 | 1H NMR (400 MHz, DMSO-d6) 12.36 (s, 1H), 10.02 (s, 1H), 8.04 (s, 1H), 7.45-6.94 (m, 3H), 6.80 (s, 1H), 5.18 (s, 1H), 4.86 (s, 1H), 4.57 (s, 2H), 3.88 (d, J = 17.0 Hz, 2H), 3.33 (s, 1H), 2.70 (d, J = 17.9 Hz, 1H), 2.10 (d, J = 104.0 Hz, 2H), 0.97 (d, J = 16.4 Hz, 6H), 0.52 (d, J = 6.6 Hz, 1H), 0.27 (d, J = 26.0 Hz, 1H). | 442.10 | +7.00 |
| (3R,5R)-5-(3-((2- (methoxymethyl) pyrazolo[1,5-a]pyrazin-4- yl)amino)-1H- pyrazol-5- yl)tetrahydrofuran-3- yl ((R*)-2,2- dimethylcyclopropyl) carbamate | 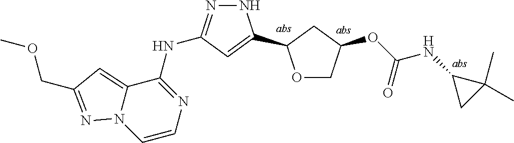 | 1H NMR (400 MHz, DMSO-d6) 12.38 (s, 1H), 10.03 (s, 1H), 8.04 (s, 1H), 7.34 (d, J = 37.3 Hz, 3H), 6.91 (d, J = 79.5 Hz, 1H), 5.13 (d, J = 43.3 Hz, 1H), 4.85 (s, 1H), 4.57 (s, 2H), 4.04-3.68 (m, 2H), 3.33 (s, 1H), 2.71 (s, 1H), 2.32-2.16 (m, 1H), 1.95 (d, J = 50.8 Hz, 1H), 0.96 (t, J = 14.8 Hz, 6H), 0.54 (t, J = 6.7 Hz, 1H), 0.27 (d, J = 27.4 Hz, 1H). | 442.20 | +10.00 |
| (3R,5R)-5-(3-((2- (methoxymethyl) pyrazolo[1,5-a]pyrazin-4- yl)amino)-1H- pyrazol-5- yl)tetrahydrofuran-3- yl ((S)-sec- butyl)carbamate | 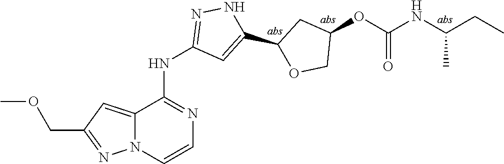 [SMILES: CC[C@H](C)NC(=O)O[C@H]1CO[C@@H](c2cc(Nc3nccn4nc(COC)cc34)n[nH]2)C1] | 1H NMR (400 MHz, DMSO-d6) 10.10 (s, 1H), 8.04 (d, J = 4.8 Hz, 1H), 7.39 (d, J = 4.8 Hz, 1H), 7.27 (s, 1H), 7.07 (d, J = 8.4 Hz, 1H), 6.68 (s, 1H), 5.18 (s, 1H), 4.82 (d, J = 8.0 Hz, 1H), 4.57 (s, 2H), 3.87 (q, J = 7.0, 5.4 Hz, 2H), 3.42- 3.38 (m, 1H), 3.33 (s, 1H), 2.72 (dd, J = 13.9, 7.0 Hz, 1H), 1.96 (d, J = 24.1 Hz, 1H), 1.88 (s, 1H), 1.44-1.20 (m, 2H), 1.01 (d, J = 6.6 Hz, 3H), 0.82 (t, J = 7.4 Hz, 3H). | 430.15 | +19.00 |
| (3R,5R)-5-(3-((2- (methoxymethyl) pyrazolo[1,5-a]pyrazin-4- yl)amino)-1H- pyrazol-5- yl)tetrahydrofuran-3- yl (1-(1-methyl-1H- pyrazol-4- yl)cyclopropyl) carbamate | 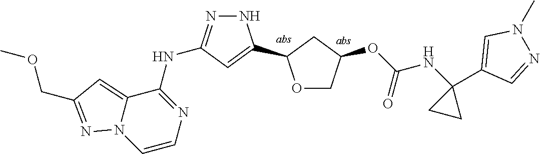 | 1H NMR (400 MHz, DMSO-d6) 12.37 (s, 1H), 10.03 (s, 1H), 8.04 (s, 1H), 7.96 (s, 1H), 7.68- 7.24 (m, 3H), 7.16 (s, 1H), 6.81 (s, 1H), 5.18 (s, 1H), 4.86 (s, 1H), 4.57 (s, 2H), 3.98-3.80 (m, 2H), 3.73 (s, 3H), 3.31 (s, 3H), 2.71 (s, 1H), 1.98 (s, 1H), 1.01 (s, 2H), 0.93-0.86 (m, 2H). | 494.20 | −4 |
| (3R,5R)-5-(3-((2- (methoxymethyl) pyrazolo[1,5-a]pyrazin-4- yl)amino)-1H- pyrazol-5- yl)tetrahydrofuran-3- yl ((S)-1-(oxetan-3- yl)ethyl)carbamate | 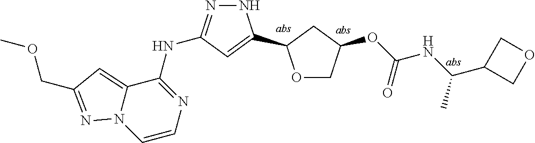 [SMILES: COCc1cc2c(Nc3cc([C@H]4C[C@@H](OC(=O)N[C@@H](C)C5COC5)CO4)[nH]n3)nccn2n1] | 1H NMR (400 MHz, DMSO-d6) 12.39 (s, 1H), 10.03 (s, 1H), 8.04 (s, 1H), 7.39 (d, J = 4.8 Hz, 1H), 7.32 (m, 2H), 6.81 (s, 1H), 5.20 (s, 1H), 4.86 (s, 1H), 4.55 (m, 4H), 4.35 (t, J = 6.1 Hz, 1H), 4.26 (t, J = 6.2 Hz, 1H), 3.81 (m, 3H), 3.33 (s, 3H), 2.92 (q, J = 7.5 Hz, 1H), 2.74 (s, 1H), 1.99 (s, 1H), 0.96 (d, J = 6.6 Hz, 3H). | 458.20 | +18 |
| (3R,5R)-5-(3-((2- (methoxymethyl) pyrazolo[1,5-a]pyrazin-4- yl)amino)-1H- pyrazol-5- yl)tetrahydrofuran-3- yl ((S)-1-(3- methyloxetan-3- yl)ethyl)carbamate | 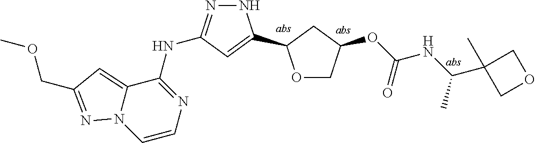 [SMILES: COCc1cc2c(Nc3cc([C@H]4C[C@@H](OC(=O)N[C@@H](C)C5(C)COC5)CO4)[nH]n3)nccn2n1] | 1H NMR (400 MHz, DMSO-d6) 12.37 (s, 1H), 10.03 (s, 1H), 8.05 (s, 1H), 7.48-7.08 (m, 3H), 6.79 (s, 1H), 5.19 (s, 1H), 4.85 (s, 1H), 4.57 (s, 2H), 4.45 (d, J = 5.7 Hz, 1H), 4.32 (d, J = 5.9 Hz, 1H), 4.09 (t, J = 5.7 Hz, 2H), 3.88 (ddd, J = 21.5, 9.8, 6.1 Hz, 3H), 3.33 (s, 3H), 2.85-2.62 (m, 1H), 2.03 (d, J = 36.8 Hz, 1H), 1.18 (s, 3H), 0.96 (d, J = 6.8 Hz, 3H). | 472.25 | +9 |
| (3R,5R)-5-(3-((2- (methoxymethyl) pyrazolo[1,5-a]pyrazin-4- yl)amino)-1H- pyrazol-5- yl)tetrahydrofuran-3- yl ((R)-1-(3- methyloxetan-3- yl)ethyl)carbamate | 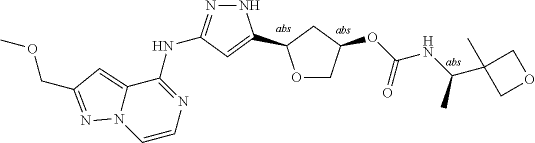 [SMILES: COCc1cc2c(Nc3cc([C@H]4C[C@@H](OC(=O)N[C@H](C)C5(C)COC5)CO4)[nH]n3)nccn2n1] | 1H NMR (400 MHz, DMSO-d6) 12.39 (s, 1H), 10.03 (s, 1H), 8.04 (s, 1H), 7.55-7.04 (m, 3H), 6.81 (s, 1H), 5.20 (s, 1H), 4.86 (s, 1H), 4.57 (s, 2H), 4.46 (d, J = 5.8 Hz, 1H), 4.33 (d, J = 5.9 Hz, 1H), 4.11 (dd, J = 5.8, 2.7 Hz, 2H), 4.02- 3.66 (m, 3H), 3.33 (s, 3H), 2.83-2.62 (m, 1H), 1.99 (s, 1H), 1.20 (s, 3H), 0.95 (d, J = 6.8 Hz, 3H). | 472.25 | +4 |
| (3R,5R)-5-(5-((2- (methoxymethyl) pyrazolo[1,5-a]pyrazin-4- yl)amino)-1H- pyrazol-3- yl)tetrahydrofuran-3- yl ((1S,2S)-2- methylcyclopropyl) carbamate | 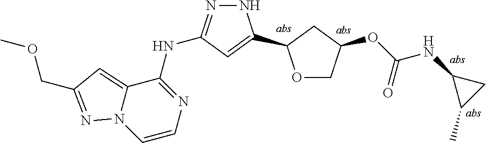 [SMILES: COCc1cc2c(Nc3cc([C@H]4C[C@@H](OC(=O)N[C@H]5C[C@@H]5C)CO4)[nH]n3)nccn2n1] | 1H NMR (400 MHz, DMSO-d6) 12.36 (s, 1H), 10.03 (s, 1H), 8.04 (s, 1H), 7.42-7.34 (m, 2H), 7.28 (s, 1H), 6.80 (s, 1H), 5.18 (dt, J = 7.8, 3.8 Hz, 1H), 4.85 (s, 1H), 4.57 (s, 2H), 3.87 (s, 2H), 3.33 (s, 3H), 2.71 (s, 1H), 2.15 (s, 1H), 1.97 (s, 1H), 0.98 (s, J = 6.0 Hz, 3H), 0.84 (s, 1H), 0.55 (s, 1H), 0.35 (d, J = 6.8 Hz, 1H). | 428.15 | 60 |
| (3R,5R)-5-(3-((2- (methoxymethyl) pyrazolo[1,5-a]pyrazin-4- yl)amino)-1H- pyrazol-5- yl)tetrahydrofuran-3- yl bicyclo[2.2.2]octan-1- ylcarbamate | 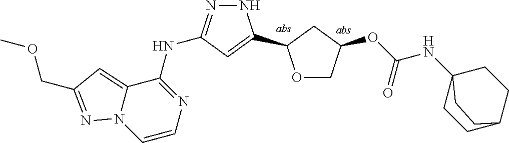 [SMILES: COCc1cc2c(Nc3cc([C@H]4C[C@@H](OC(=O)NC56CCC(CC5)CC6)CO4)[nH]n3)nccn2n1] | 1H NMR (400 MHz, DMSO-d6) δ 12.34 (s, 1H), 10.03 (s, 1H), 8.05 (s, 1H), 7.39 (d, J = 4.8 Hz, 1H), 7.28 (s, 1H), 6.78 (s, 2H), 5.13 (s, 1H), 4.84 (s, 1H), 4.57 (s, 2H), 3.84 (s, 2H), 3.33 (s, 3H), 2.68 (s, 1H), 1.95 (s, 1H), 1.68 (s, 6H), 1.60-1.45 (m, 7H). | 482.20 | 3 |
| (3R,5R)-5-(3-((2- (methoxymethyl) pyrazolo[1,5-a]pyrazin-4- yl)amino)-1H- pyrazol-5- yl)tetrahydrofuran-3- yl oxetan-3- ylcarbamate | 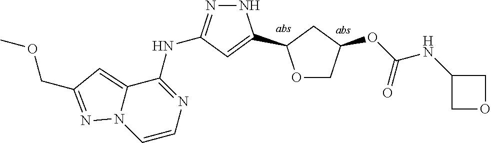 [SMILES: COCc1cc2c(Nc3cc([C@H]4C[C@@H](OC(=O)NC5COC5)CO4)[nH]n3)nccn2n1] | 1H NMR (400 MHz, DMSO-d6) 12.39 (s, 2H), 10.03 (s, 2H), 8.05 (d, J = 13.3 Hz, 7H), 7.40 (d, J = 4.8 Hz, 4H), 7.30 (s, 3H), 6.81 (s, 3H), 5.19 (s, 4H), 4.87 (s, 2H), 4.65 (s, 9H), 4.57 (s, 10H), 4.46-4.41 (m, 7H), 4.32 (s, 1H), 3.91 (d, J = 9.8 Hz, 5H), 3.86 (s, 3H), 3.33 (s, 28H), 2.72 (s, 2H), 2.00 (s, 2H). | 430.10 | +26.00 |
| (3R,5R)-5-(3-((2- (methoxymethyl) pyrazolo[1,5-a]pyrazin-4- yl)amino)-1H- pyrazol-5- yl)tetrahydrofuran-3- yl (3-methyloxetan-3- yl)carbamate | 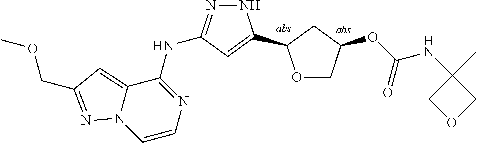 [SMILES: COCc1cc2c(Nc3cc([C@H]4C[C@@H](OC(=O)NC5(C)COC5)CO4)[nH]n3)nccn2n1] | 1H NMR (400 MHz, DMSO-d6) 12.39 (s, 1H), 10.04 (s, 1H), 8.05 (s, 1H), 7.81 (s, 1H), 7.40 (d, J = 4.8 Hz, 1H), 7.29 (s, 1H), 6.81 (s, 1H), 5.20 (d, J = 3.3 Hz, 1H), 4.87 (s, 1H), 4.57 (s, 4H), 4.25 (d, J = 6.0 Hz, 2H), 3.91 (d, J = 10.2 Hz, 1H), 3.88-3.81 (m, 1H), 3.30 (s, 3H), 2.72 (s, 1H), 2.01 (s, 1H), 1.48 (s, 3H). | 444.15 | 37 |
| (3R,5R)-5-(3-((2- (methoxymethyl) pyrazolo[1,5-a]pyrazin-4- yl)amino)-1H- pyrazol-5- yl)tetrahydrofuran-3- yl (1-methoxy-2- methylpropan-2- yl)carbamate | 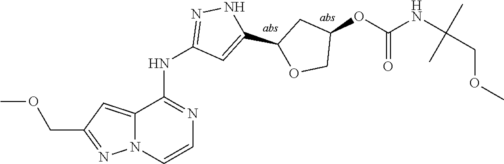 | 1H NMR (400 MHz, DMSO-d6) 12.36 (s, 1H), 10.02 (s, 1H), 8.04 (s, 1H), 7.39 (d, J = 4.8 Hz, 1H), 7.29 (s, 1H), 6.83 (s, 2H), 5.16 (s, 1H), 4.86 (s, 1H), 4.57 (s, 2H), 3.86 (q, J = 7.3, 5.8 Hz, 2H), 3.35 (s, 2H), 3.33 (s, 3H), 3.24 (s, 3H), 2.71 (s, 1H), 1.96 (s, 1H), 1.17 (s, 6H). | 460.15 | 19 |
| (3R,5R)-5-(3-((2- (methoxymethyl) pyrazolo[1,5-a]pyrazin-4- yl)amino)-1H- pyrazol-5- yl)tetrahydrofuran-3- yl (2-(tetrahydro-2H- pyran-4- yl)ethyl)carbamate | 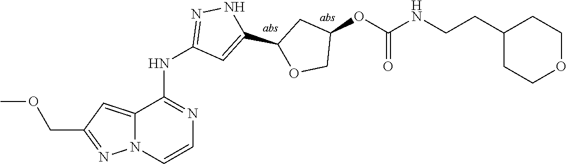 [SMILES: COCc1cc2c(Nc3cc([C@H]4C[C@@H](OC(=O)NCCC5CCOCC5)CO4)[nH]n3)nccn2n1] | 1H NMR (400 MHz, DMSO-d6) 12.38 (s, 1H), 10.02 (s, 1H), 8.04 (s, 1H), 7.40 (d, J = 4.9 Hz, 1H), 7.29 (s, 1H), 7.19 (t, J = 5.7 Hz, 1H), 6.80 (s, 1H), 5.18 (s, 1H), 4.86 (s, 1H), 4.57 (s, 2H), 3.88 (q, J = 7.2, 4.0 Hz, 2H), 3.78 (d, J = 11.6 Hz, 2H), 3.31 (s, 3H), 3.27-3.16 (m, 2H), 3.01 (q, J = 6.7 Hz, 2H), 2.96-2.69(m, 1H), 2.03 (d, J = 40.4 Hz, 1H), 1.64-1.41 (m, 3H), 1.32 (q, J = 6.9 Hz, 2H), 1.10 (qd, J = 11.9, 4.4 Hz, 2H). | 486.25 | 9 |
| (3R,5R)-5-(3-((2- (methoxymethyl) pyrazolo[1,5-a]pyrazin-4- yl)amino)-1H- pyrazol-5- yl)tetrahydrofuran-3- yl [1,1′- bi(cyclopropan)]-1- ylcarbamate | 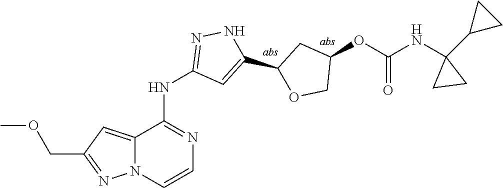 [SMILES: COCc1cc2c(Nc3cc([C@H]4C[C@@H](OC(=O)NC5(C6CC6)CC5)CO4)[nH]n3)nccn2n1] | 1H NMR (400 MHz, DMSO-d6) 12.36 (s, 1H), 10.02 (s, 1H), 8.04 (s, 1H), 7.56 (s, 1H), 7.42- 7.23 (m, 2H), 6.80 (s, 1H), 5.17 (s, 1H), 4.85 (s, 1H), 4.57 (s, 2H), 4.06-3.60 (m, 2H), 3.33 (s, 3H), 2.85-2.63 (m, 1H), 1.95 (s, 1H), 1.21 (s, 1H), 0.53 (d, J = 19.7 Hz, 2H), 0.45 (s, 2H), 0.30 (d, J = 8.1 Hz, 2H), 0.17-0.05 (m, 2H). | 454.20 | +6 |
| (3R,5R)-5-(3-((2- (methoxymethyl) pyrazolo[1,5-a]pyrazin-4- yl)amino)-1H- pyrazol-5- yl)tetrahydrofuran-3- yl (1- cyanocyclopropyl) carbamate | 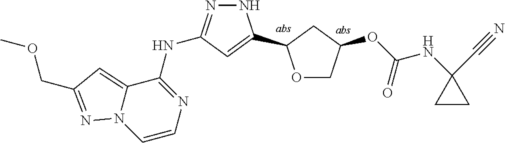 [SMILES: COCc1cc2c(Nc3cc([C@H]4C[C@@H](OC(=O)NC5(C#N)CC5)CO4)[nH]n3)nccn2n1] | 1H NMR (400 MHz, DMSO-d6) 12.40 (s, 1H), 10.02 (s, 1H), 8.39 (s, 1H), 8.04 (s, 1H), 7.39 (s, 1H), 7.29 (s, 1H), 6.80 (s, 1H), 5.27 (s, 1H), 4.88 (s, 1H), 4.57 (s, 2H), 3.90 (s, 1H), 3.33 (s, 3H), 2.76 (s, 1H), 2.00 (s, 1H), 1.45 (q, J = 5.1 Hz, 2H), 1.16 (q, J = 5.2 Hz, 2H). | 439.15 | +8 |
| (3R,5R)-5-(3-((2- (methoxymethyl) pyrazolo[1,5-a]pyrazin-4- yl)amino)-1H- pyrazol-5- yl)tetrahydrofuran-3- yl ((1s,4S)-4- methoxycyclohexyl) carbamate | 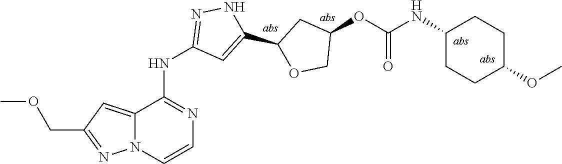 | 1H NMR (400 MHz, DMSO-d6) 12.35 (s, 1H), 10.02 (s, 1H), 8.04 (s, 1H), 7.39 (d, J = 4.8 Hz, 1H), 7.29 (s, 1H), 7.18 (d, J = 7.8 Hz, 1H), 6.80 (s, 1H), 5.18 (s, 1H), 4.85 (s, 1H), 4.57 (s, 2H), 3.88 (t, J = 10.2 Hz, 2H), 3.33 (s, 3H), 3.25 (s, 1H), 3.17 (s, 3H), 3.07 (s, 1H), 2.71 (s, 1H), 1.98 (s, 1H), 1.76 (s, 2H), 1.46 (d, J = 22.9 Hz, 6H). | 486.25 | +9 |
| N-(5-((2R,4R)-4-((4- isopropyl-4H-1,2,4- triazol-3- yl)oxy)tetrahydrofuran- 2-yl)-1H-pyrazol-3- yl)-1-methyl-3- ((trifluoromethoxy) methyl)-1H-pyrazole- | 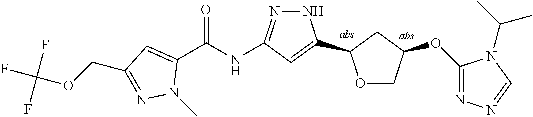 [SMILES: CC(C)n1cnnc1O[C@H]1CO[C@@H](c2cc(NC(=O)c3cc(COC(F)(F)F)nn3C)n[nH]2)C1] | 1H NMR (400 MHz, DMSO-d6) 12.54 (s, 1H), 10.92 (s, 1H), 8.27 (d, J = 1.1 Hz, 1H), 7.30 (s, 1H), 6.60 (s, 1H), 5.48 (t, J = 5.3 Hz, 1H), 5.12 (s, 3H), 4.19-4.00 (m, 6H), 2.79 (dt, J = 14.6, 7.5 Hz, 1H), 2.36-2.19 (m, 1H), 1.28 (dd, J = 6.9, 2.5 Hz, 6H). | 485.20 | 22 |
| 5-carboxamide | | | | |
| (3R,5R)-5-(3-((2- (methoxymethyl) pyrazolo[1,5-a]pyrazin-4- yl)amino)-1H- pyrazol-5- yl)tetrahydrofuran-3- yl ((1R,2R)-2- methylcyclopropyl) carbamate | 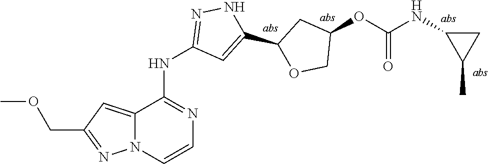 [SMILES: COCc1cc2c(Nc3cc([C@H]4C[C@@H](OC(=O)N[C@@H]5C[C@H]5C)CO4)[nH]n3)nccn2n1] | 1H NMR (400 MHz, DMSO-d6) 12.36 (s, 1H), 10.02 (s, 1H), 8.04 (s, 1H), 7.48-6.99 (m, 3H), 6.79 (s, 1H), 5.18 (s, 1H), 4.86 (s, 1H), 4.57 (s, 2H), 3.87 (s, 2H), 3.33 (s, 3H), 2.81-2.64 (m, 1H), 2.15 (s, 1H), 1.97 (s, 1H), 0.98 (d, J = 6.0 Hz, 3H), 0.76 (s, 1H), 0.55 (s, 1H), 0.35 (s, 1H). | 428.15 | −8.00 |
| (3R,5R)-5-(3-((2- (methoxymethyl) pyrazolo[1,5-a]pyrazin-4- yl)amino)-1H- pyrazol-5- yl)tetrahydrofuran-3- yl (1- ethylcyclopropyl) carbamate | 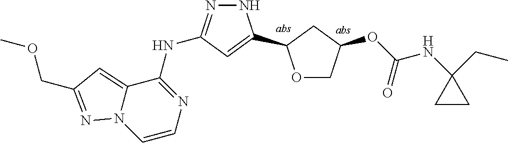 [SMILES: CCC1(NC(=O)O[C@H]2CO[C@@H](c3cc(Nc4nccn5nc(COC)cc45)n[nH]3)C2)CC1] | 1H NMR (400 MHz, DMSO-d6) 12.37 (s, 1H), 10.03 (s, 1H), 8.04 (s, 1H), 7.53 (s, 1H), 7.34 (d, J = 35.3 Hz, 2H), 6.80 (s, 1H), 5.16 (s, 1H), 4.91 (d, J = 44.5 Hz, 1H), 4.57 (s, 2H), 3.86 (s, 2H), 3.33 (s, 3H), 2.71 (s, 1H), 2.02 (d, J = 55.9 Hz, 1H), 1.44 (h, J = 7.0 Hz, 2H), 0.84 (t, J = 7.4 Hz, 2H), 0.58 (s, 2H), 0.49 (d, J = 5.1 Hz, 2H). | 442.15 | +12.00 |
| (3R,5R)-5-(3-((2- (methoxymethyl) pyrazolo[1,5-a]pyrazin-4- yl)amino)-1H- pyrazol-5- yl)tetrahydrofuran-3- yl ((R)-1-(oxetan-3- yl)ethyl)carbamate | 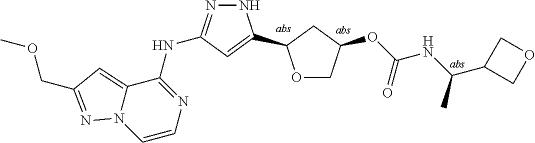 [SMILES: COCc1cc2c(Nc3cc([C@H]4C[C@@H](OC(=O)N[C@H](C)C5COC5)CO4)[nH]n3)nccn2n1] | 1H NMR (400 MHz, DMSO-d6) 12.40 (s, 1H), 10.04 (s, 1H), 8.04 (s, 1H), 7.39 (d, J = 4.7 Hz, 1H), 7.35-7.04 (m, 2H), 6.80 (s, 1H), 5.19 (s, 1H), 4.85 (s, 1H), 4.68-4.41 (m, 4H), 4.33 (t, J = 6.2 Hz, 1H), 4.25 (t, J = 6.2 Hz, 1H), 4.00- 3.74 (m, 3H), 3.33 (s, 3H), 2.90 (q, J = 7.4 Hz, 1H), 2.73 (s, 1H), 1.98 (s, 1H), 0.96 (d, J = 6.6 Hz, 3H). | 458.20 | +7.00 |
| N-(5-((1S,3R)-3-((4- isopropyl-4H-1,2,4- triazol-3- yl)oxy)cyclopentyl)- 1H-pyrazol-3-yl)-2- (methoxymethyl) pyrazolo[1,5-a]pyrazin-4- amine | 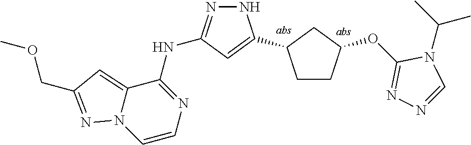 [SMILES: COCc1cc2c(Nc3cc([C@H]4CC[C@@H](Oc5nncn5C(C)C)C4)[nH]n3)nccn2n1] | 1H NMR (400 MHz, DMSO-d6) 12.17 (s, 1H), 9.98 (s, 1H), 8.23 (s, 1H), 8.01 (d, J = 4.8 Hz, 1H), 7.48-7.14 (m, 2H), 6.63 (s, 1H), 5.32 (d, J = 7.1 Hz, 1H), 4.56 (s, 2H), 4.16 (p, J = 6.7 Hz, 1H), 3.33 (s, 3H), 3.23 (t, J = 8.4 Hz, 1H), 2.73- 2.56 (m, 1H), 2.13 (dd, J = 11.2, 6.3 Hz, 1H), 2.04 (dt, J = 16.0, 5.8 Hz, 2H), 1.99-1.79 (m, 2H), 1.30 (dd, J = 6.7, 4.7 Hz, 6H). | 438.20 | +55 |
| (3R,5R)-5-(3-((2- (methoxymethyl) pyrazolo[1,5-a]pyrazin-4- yl)amino)-1H- pyrazol-5- yl)tetrahydrofuran-3- yl (2-cyanopropan-2- yl)carbamate | 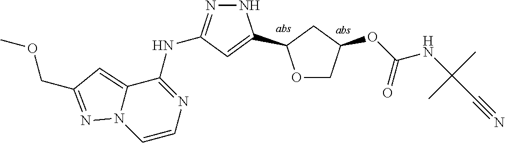 [SMILES: COCc1cc2c(Nc3cc([C@H]4C[C@@H](OC(=O)NC(C)(C)C#N)CO4)[nH]n3)nccn2n1] | 1H NMR (400 MHz, DMSO-d6) 12.40 (s, 1H), 10.36-9.52 (m, 1H), 8.06 (d, J = 14.2 Hz, 2H), 7.39 (d, J = 4.7 Hz, 1H), 7.06 (d, J = 190.4 Hz, 2H), 5.28 (s, 1H), 4.89 (s, 1H), 4.57 (s, 2H), 3.92 (t, J = 11.4 Hz, 2H), 3.33 (s, 3H), 2.73 (d, J = 42.0 Hz, 1H), 2.01 (s, 1H), 1.56 (s, 6H). | 441.15 | +26 |
| (3R,5R)-5-(3-(1- methyl-3- ((trifluoromethoxy) methyl)-1H-pyrazole-5- carboxamido)-1H- pyrazol-5- yl)tetrahydrofuran-3- yl isopropylcarbamate | 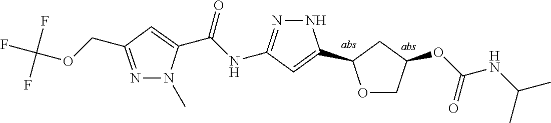 [SMILES: CC(C)NC(=O)O[C@H]1CO[C@@H](c2cc(NC(=O)c3cc(COC(F)(F)F)nn3C)n[nH]2)C1] | 1H NMR (400 MHz, DMSO-d6) 12.52 (s, 1H), 10.92 (s, 1H), 7.29 (s, 1H), 7.11 (d, J = 7.8 Hz, 1H), 6.58 (s, 1H), 5.15 (d, J = 21.9 Hz, 3H), 4.86 (s, 1H), 4.10 (s, 3H), 3.87 (q, J = 7.3, 5.6 Hz, 2H), 3.59 (h, J = 6.7 Hz, 1H), 2.82-2.62 (m, 1H), 1.93 (d, J = 18.1 Hz, 1H), 1.05 (d, J = 6.6 Hz, 6H). | 461.20 | 62 |
| (3R,5R)-5-(3-(1- methyl-3- ((trifluoromethoxy) methyl)-1H-pyrazole-5- carboxamido)-1H- pyrazol-5- yl)tetrahydrofuran-3- yl tert-butylcarbamate | 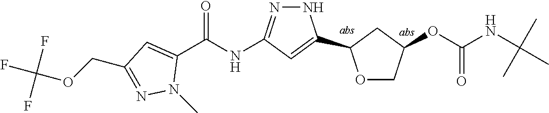 [SMILES: Cn1nc(COC(F)(F)F)cc1C(=O)Nc1cc([C@H]2C[C@@H](OC(=O)NC(C)(C)C)CO2)[nH]n1] | 1H NMR (400 MHz, DMSO-d6) 12.50 (s, 1H), 10.91 (s, 1H), 7.29 (s, 1H), 6.93 (s, 1H), 6.59 (s, 1H), 5.13 (d, J = 11.4 Hz, 3H), 4.87 (d, J = 7.6 Hz, 1H), 4.10 (s, 3H), 3.85 (d, J = 4.8 Hz, 2H), 2.71 (dt, J = 14.5, 7.5 Hz, 1H), 1.94 (s, 1H), 1.22 (s, 9H). | 475.10 | 29 |
| (3R,5R)-5-(3-((2- (methoxymethyl) pyrazolo[1,5-a]pyrazin-4- yl)amino)-1H- pyrazol-5- yl)tetrahydrofuran-3- yl (2- oxabicyclo[2.2.2] octan-4-yl)carbamate | 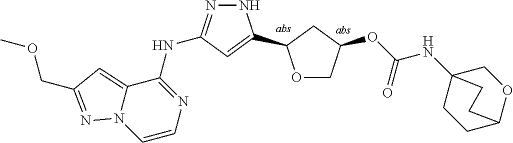 [SMILES: COCc1cc2c(Nc3cc([C@H]4C[C@@H](OC(=O)NC56CCC(CC5)OC6)CO4)[nH]n3)nccn2n1] | 1H NMR (400 MHz, DMSO-d6) 12.36 (s, 1H), 10.05 (s, 1H), 8.05 (s, 1H), 7.39 (d, J = 4.8 Hz, 1H), 7.28 (s, 1H), 7.08 (m, 1H), 6.79 (s, 1H), 5.15 (s, 1H), 4.84 (s, 1H), 4.57 (s, 2H), 3.85 (m, 2H), 3.74 (s, 2H), 3.63 (s, 1H), 3.34 (s, 3H), 2.70 (d, J = 8.5 Hz, 1H), 1.90 (s, 5H), 1.75 (s, 2H), 1.60 (d, J = 13.5 Hz, 2H). | 484.25 | +10 |
| (3R,5R)-5-(3-((2- ((difluoromethoxy) methyl)pyrazolo[1,5- a]pyrazin-4- yl)amino)-1H- pyrazol-5- yl)tetrahydrofuran-3- yl (1- methylcyclopropyl) | 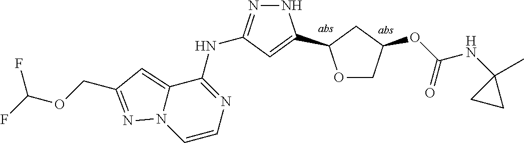 [SMILES: CC1(NC(=O)O[C@H]2CO[C@@H](c3cc(Nc4nccn5nc(COC(F)F)cc45)n[nH]3)C2)CC1] | 1H NMR (400 MHz, DMSO-d6) 12.38 (s, 1H), 10.12 (s, 1H), 8.07 (s, 1H), 7.53 (s, 1H), 7.41 (d, J = 13.4 Hz, 2H), 6.84 (t, J = 75.4 Hz, 2H), 5.17 (s, 1H), 5.09 (s, 2H), 4.86 (s, 1H), 3.86 (s, 2H), 2.71 (s, 1H), 1.95 (s, 1H), 1.25 (s, 3H), 0.61 (s, 2H), 0.48 (q, J = 4.6 Hz, 2H). | 464.10 | 6 |
| carbamate | | | | |
| (3R,5R)-5-(3-((2- (methoxymethyl) pyrazolo[1,5-a]pyrazin-4- yl)amino)-1H- pyrazol-5- yl)tetrahydrofuran-3- yl (3- fluorobicyclo[1.1.1] pentan-1-yl)carbamate | 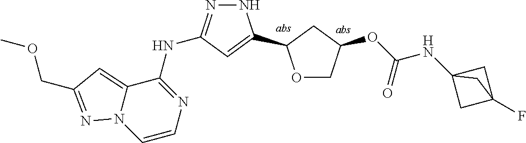 [SMILES: COCc1cc2c(Nc3cc([C@H]4C[C@@H](OC(=O)NC56CC(F)(C5)C6)CO4)[nH]n3)nccn2n1] | 1H NMR (400 MHz, DMSO-d6) 12.39 (s, 1H), 10.03 (s, 1H), 8.09 (d, J = 37.9 Hz, 2H), 7.34 (d, J = 35.7 Hz, 2H), 6.81 (s, 1H), 5.23-5.17 (m, 1H), 4.88 (s, 1H), 4.57 (s, 2H), 3.88 (s, 2H), 3.33 (s, 3H), 2.72 (s, 1H), 2.25 (s, 6H), 1.98 (s, 1H). | 458.10 | 10 |
| (3R,5R)-5-(3-((2- (methoxymethyl) pyrazolo[1,5-a]pyrazin-4- yl)amino)-1H- pyrazol-5- yl)tetrahydrofuran-3- yl (3- chlorobicyclo[1.1.1] pentan-1-yl)carbamate | 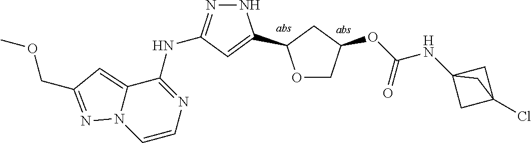 [SMILES: COCc1cc2c(Nc3cc([C@H]4C[C@@H](OC(=O)NC56CC(Cl)(C5)C6)CO4)[nH]n3)nccn2n1] | 1H NMR (400 MHz, DMSO-d6) 12.38 (s, 1H), 10.03 (s, 1H), 8.11 (d, J = 59.0 Hz, 2H), 7.35 (d, J = 37.8 Hz, 2H), 6.81 (s, 1H), 5.19 (s, 1H), 4.88 (s, 1H), 4.57 (s, 2H), 3.88 (s, 2H), 3.33 (s, 3H), 2.71 (s, 1H), 2.31 (s, 6H), 2.24-1.72 (m, 1H). | 474.10 | 11 |
| (3R,5R)-5-(3-((2- (methoxymethyl) pyrazolo[1,5-a]pyrazin-4- yl)amino)-1H- pyrazol-5- yl)tetrahydrofuran-3- yl (2-methyl-1- morpholinopropan-2- yl)carbamate | 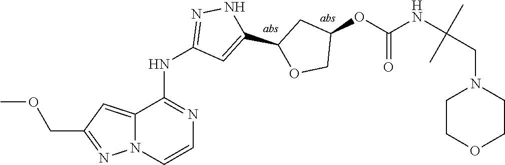 [SMILES: COCc1cc2c(Nc3cc([C@H]4C[C@@H](OC(=O)NC(C)(C)CN5CCOCC5)CO4)[nH]n3)nccn2n1] | 1H NMR (400 MHz, DMSO-d6) 8.05 (d, J = 4.7 Hz, 1H), 7.39 (d, J = 4.8 Hz, 1H), 7.27 (s, 1H), 6.81 (s, 1H), 6.69 (s, 1H), 6.04 (s, 1H), 5.15 (s, 1H), 4.84 (t, J = 7.8 Hz, 1H), 4.57 (s, 2H), 3.91-3.79 (m, 2H), 3.50 (t, J = 4.6 Hz, 4H), 3.30 (s, 3H), 2.71 (dd, J = 14.0, 7.2 Hz, 1H), 2.42 (q, J = 5.6, 4.5 Hz, 6H), 1.95 (s, 1H), 1.17 (s, 6H). | 515.35 | 2 |
| (3R,5R)-5-(3-((2- (methoxymethyl) pyrazolo[1,5-a]pyrazin-4- yl)amino)-1H- pyrazol-5- yl)tetrahydrofuran-3- yl ((S)-1,1,1- trifluoropropan-2- yl)carbamate | 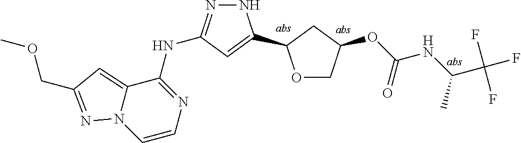 [SMILES: COCc1cc2c(Nc3cc([C@H]4C[C@@H](OC(=O)N[C@@H](C)C(F)(F)F)CO4)[nH]n3)nccn2n1] | H NMR (400 MHz, DMSO-d6) 12.39 (s, 1H), 10.03 (s, 1H), 8.01 (d, J = 9.1 Hz, 2H), 7.39 (d, J = 4.8 Hz, 1H), 7.30 (s, 1H), 6.81 (s, 1H), 5.24 (dp, J = 7.8, 2.5 Hz, 1H), 4.87 (s, 1H), 4.57 (s, 2H), 4.29 (h, J = 7.5 Hz, 1H), 3.90 (q, J = 7.6, 4.7 Hz, 2H), 3.33 (s, 3H), 2.76 (s, 1H), 2.01 (s, 1H), 1.23 (d, J = 7.0 Hz, 3H). | 470.20 | 10 |
| (3R,5R)-5-(3-((2- (methoxymethyl) pyrazolo[1,5-a]pyrazin-4- yl)amino)-1H- pyrazol-5- yl)tetrahydrofuran-3- yl (®-1,1,1- trifluoropropan-2- yl)carbamate | 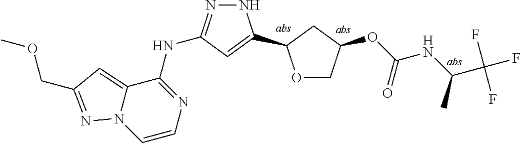 [SMILES: COCc1cc2c(Nc3cc([C@H]4C[C@@H](OC(=O)N[C@H](C)C(F)(F)F)CO4)[nH]n3)nccn2n1] | 1H NMR (400 MHz, DMSO-d6) 12.40 (s, 1H), 10.03 (s, 1H), 8.02 (d, J = 10.8 Hz, 2H), 7.43- 7.28 (m, 2H), 6.82 (s, 1H), 5.25 (s, 1H), 4.86 (s, 1H), 4.57 (s, 2H), 4.27 (dt, J = 15.2, 7.7 Hz, 1H), 3.91 (t, J = 14.0 Hz, 2H), 3.33 (s, 3H), 2.77 (s, 1H), 1.99 (s, 1H), 1.24 (d, J = 7.0 Hz, 3H). | 470.20 | 18 |
| (3R,5R)-5-(3-((2- (methoxymethyl) pyrazolo[1,5-a]pyrazin-4- yl)amino)-1H- pyrazol-5- yl)tetrahydrofuran-3- yl ((R*)-2,2-difluoro- 1-methylcyclopropyl) carbamate | 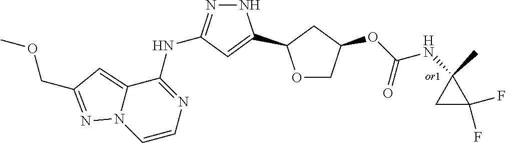 [SMILES: COCc1cc2c(Nc3cc([C@H]4C[C@@H](OC(=O)N[C@@]5(C)CC5(F)F)CO4)[nH]n3)nccn2n1] | 1H NMR (400 MHz, DMSO-d6) 12.40 (s, 1H), 10.05 (s, 1H), 8.16-7.98 (m, 1H), 7.88 (d, J = 3.3 Hz, 1H), 7.45-7.16 (m, 2H), 6.79 (s, 1H), 5.21 (dt, J = 7.2, 3.8 Hz, 1H), 4.85 (s, 1H), 4.57 (s, 2H), 3.86 (d, J = 4.6 Hz, 2H), 3.33 (s, 3H), 2.84-2.67 (m, 1H), 2.00 (s, 1H), 1.51 (d, J = 14.9 Hz, 2H), 1.35 (s, 3H). | 464.20 | 19 |
| (3R,5R)-5-(3-((2- (methoxymethyl) pyrazolo[1,5-a]pyrazin-4- yl)amino)-1H- pyrazol-5- yl)tetrahydrofuran-3- yl ((R*)-2,2-difluoro- 1-methylcyclopropyl) carbamate | 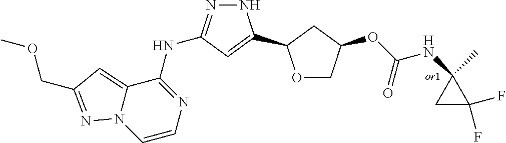 [SMILES: COCc1cc2c(Nc3cc([C@H]4C[C@@H](OC(=O)N[C@]5(C)CC5(F)F)CO4)[nH]n3)nccn2n1] | 1H NMR (400 MHz, DMSO-d6) 12.40 (s, 1H), 10.09 (s, 1H), 7.97 (dd, J = 64.5, 4.0 Hz, 2H), 7.43-7.13 (m, 2H), 6.74 (s, 1H), 5.21 (d, J = 7.3 Hz, 1H), 4.84 (s, 1H), 4.57 (s, 2H), 4.02- 3.75 (m, 2H), 3.33 (s, 3H), 2.76 (dd, J = 26.5, 6.6 Hz, 1H), 2.04 (d, J = 52.9 Hz, 1H), 1.64- 1.43 (m, 2H), 1.43-1.17 (m, 3H). | 464.20 | 10 |
| (3R,5R)-5-(3-((2- (methoxymethyl) pyrazolo[1,5-a]pyrazin-4- yl)amino)-1H- pyrazol-5- yl)tetrahydrofuran-3- yl 2,2- dimethylazetidine-1- carboxylate | 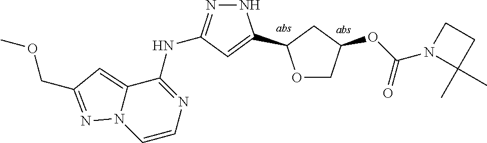 | 1H NMR (400 MHz, DMSO-d6) 12.39 (s, 1H), 10.02 (s, 1H), 8.04 (s, 1H), 7.28 (s, 2H), 6.81 (s, 1H), 5.14 (m, 1H), 4.95 (s, 1H), 4.57 (s, 2H), 3.88 (d, J = 2.7 Hz, 2H), 3.75 (h, J = 8.5 Hz, 1H), 3.66 (t, J = 7.6 Hz, 1H), 3.33 (s, 3H), 2.68 (m, 1H), 2.07 (s, 1H), 1.94 (t, J = 7.6 Hz, 2H), 1.38 (d, J = 3.4 Hz, 3H), 1.30 (s, 3H). | 422.20 | +19 |
| (3R,5R)-5-(3-((2- (methoxymethyl) pyrazolo[1,5-a]pyrazin-4- yl)amino)-1H- pyrazol-5- yl)tetrahydrofuran-3- yl (1- ethynylcyclopropyl) carbamate | 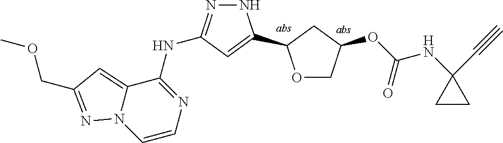 [SMILES: C#CC1(NC(=O)O[C@H]2CO[C@@H](c3cc(Nc4nccn5nc(COC)cc45)n[nH]3)C2)CC1] | 1H NMR (400 MHz, DMSO-d6) 12.37 (s, 1H), 10.03 (s, 1H), 8.02 (d, J = 13.3 Hz, 2H), 7.34 (d, J = 39.8 Hz, 2H), 6.79 (s, 1H), 5.21 (s, 1H), 4.86 (s, 1H), 4.57 (s, 2H), 3.87 (s, 2H), 3.33 (s, 3H), 2.98 (s, 1H), 2.81-2.60 (m, 1H), 1.96 (s, 1H), 1.05 (q, J = 4.4, 3.4 Hz, 2H), 0.97 (s, 2H). | 438.15 | +10 |
| rel-(3R,5R)-5-(3-((2- (methoxymethyl) pyrazolo[1,5-a]pyrazin-4- yl)amino)-1H- pyrazol-5- yl)tetrahydrofuran-3- yl ((R*)-4,4,4- trifluorobutan-2- yl)carbamate | 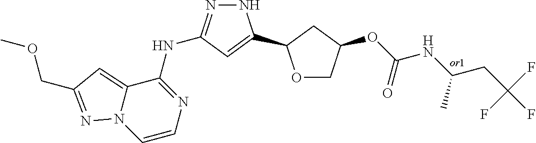 [SMILES: COCc1cc2c(Nc3cc([C@H]4C[C@@H](OC(=O)N[C@@H](C)CC(F)(F)F)CO4)[nH]n3)nccn2n1] | 1H NMR (400 MHz, DMSO-d6) 12.41 (s, 1H), 10.04 (s, 1H), 8.04 (s, 1H), 7.44-7.35 (m, 2H), 7.29 (s, 1H), 6.80 (s, 1H), 5.19 (dt, J = 7.5, 3.6 Hz, 1H), 4.85 (s, 1H), 4.57 (s, 2H), 3.86 (s, 3H), 3.33 (s, 3H), 2.72 (s, 1H), 2.46-2.28 (m, 2H), 1.99 (s, 1H), 1.13 (d, J = 6.8 Hz, 3H). | 484.10 | 12 |
| (3R,5R)-5-(3-((2- (methoxymethyl) pyrazolo[1,5-a]pyrazin-4- yl)amino)-1H- pyrazol-5- yl)tetrahydrofuran-3- yl 3- azabicyclo[3.1.1] heptane-3-carboxylate | 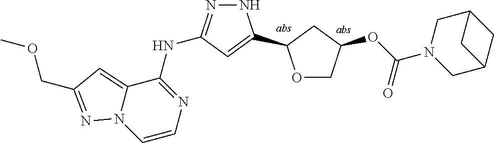 [SMILES: COCc1cc2c(Nc3cc([C@H]4C[C@@H](OC(=O)N5CC6CC(C6)C5)CO4)[nH]n3)nccn2n1] | 1H NMR (400 MHz, DMSO-d6) 12.35 (s, 1H), 10.02 (s, 1H), 8.02 (s, 1H), 7.32 (d, J = 14.6 Hz, 2H), 6.82 (s, 1H), 5.24 (dt, J = 6.4, 3.2 Hz, 1H), 5.04 (s, 1H), 4.57 (s, 2H), 3.97 (d, J = 10.3 Hz, 2H), 3.44 (d, J = 2.6 Hz, 4H), 3.33 (s, 3H), 2.67 (dt, J = 14.5, 7.7 Hz, 1H), 2.38 (s, 1H), 2.24 (s, 1H), 2.20-2.10 (m, 1H), 2.03 (dd, J = 15.9, 7.9 Hz, 2H), 1.22 (q, J = 12.7, 10.2 Hz, 2H). | 454.20 | +16 |
| (3R,5R)-5-(3-((2- (methoxymethyl) pyrazolo[1,5-a]pyrazin-4- yl)amino)-1H- pyrazol-5- yl)tetrahydrofuran-3- yl ((R)-1,1- difluoropropan-2- yl)carbamate | 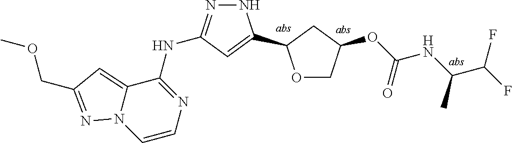 [SMILES: COCc1cc2c(Nc3cc([C@H]4C[C@@H](OC(=O)N[C@H](C)C(F)F)CO4)[nH]n3)nccn2n1] | 1H NMR (400 MHz, DMSO-d6) 12.38 (s, 1H), 10.03 (s, 1H), 8.04 (s, 1H), 7.62 (d, J = 8.5 Hz, 1H), 7.39 (d, J = 4.8 Hz, 1H), 7.22 (d, J = 59.1 Hz, 1H), 6.81 (s, 1H), 5.90 (td, J = 56.0, 3.2 Hz, 1H), 5.23 (dd, J = 8.2, 4.6 Hz, 1H), 4.86 (s, 1H), 4.57 (s, 2H), 4.00-3.79 (m, 3H), 3.33 (s, 3H), 2.75 (s, 1H), 1.99 (s, 1H), 1.11 (d, J = 7.0 Hz, 3H). | 452.20 | 10 |
| N-(5-((2R,4R)-4-((4- isopropyl-4H-1,2,4- triazol-3-yl)oxy) tetrahydrofuran- 2-yl)-1H-pyrazol-3- yl)imidazo[1,2- c]pyrimidin-5-amine | 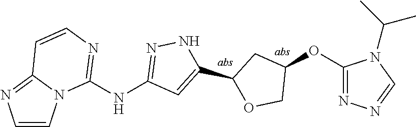 [SMILES: CC(C)n1cnnc1O[C@H]1CO[C@@H](c2cc(Nc3nccc4nccn34)n[nH]2)C1] | 1H NMR (400 MHz, DMSO-d6) 12.54 (s, 1H), 10.19 (s, 1H), 8.25 (s, 2H), 7.72-7.33 (m, 2H), 7.06-6.54 (m, 2H), 5.48 (td, J = 4.9, 2.2 Hz, 1H), 5.09 (s, 1H), 4.22-3.95 (m, 3H), 2.88-2.75 (m, 1H), 2.31 (dd, J = 14.4, 5.7 Hz, 1H), 1.25 (dd, J = 9.0, 6.5 Hz, 6H). | 396.15 | |
| (3R,5R)-5-(3-(3- ((difluoromethoxy) methyl)-1-methyl-1H- pyrazole-5- carboxamido)-1H- pyrazol-5- yl)tetrahydrofuran-3- yl (1-methylcyclopropyl) | 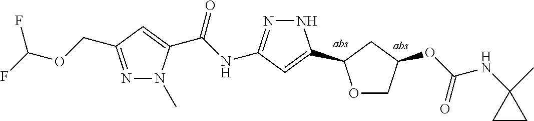 [SMILES: Cn1nc(COC(F)F)cc1C(=O)Nc1cc([C@H]2C[C@@H](OC(=O)NC3(C)CC3)CO2)[nH]n1] | 1H NMR (400 MHz, DMSO-d6) 12.51 (s, 1H), 10.86 (s, 1H), 7.51 (s, 1H), 7.21 (s, 1H), 6.99- 6.49 (m, 2H), 5.16 (s, 1H), 4.86 (s, 3H), 4.08 (s, 3H), 3.85 (s, 2H), 2.71 (dd, J = 14.0, 7.3 Hz, 1H), 1.93 (s, 1H), 1.25 (s, 3H), 0.76-0.37 (m, 4H). | 455.20 | 28 |
| carbamate | | | | |
| (3R,5R)-5-(3-(1- methyl-3- ((trifluoromethoxy) methyl)-1H-pyrazole-5- carboxamido)-1H- pyrazol-5- yl)tetrahydrofuran-3- yl ((S)-1- | 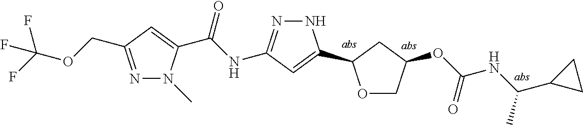 [SMILES: C[C@H](NC(=O)O[C@H]1CO[C@@H](c2cc(NC(=O)c3cc(COC(F)(F)F)nn3C)n[nH]2)C1)C1CC1] | 1H NMR (400 MHz, DMSO-d6) 12.52 (s, 1H), 10.92 (s, 1H), 7.37-7.22 (m, 1H), 7.17 (d, J = 8.4 Hz, 1H), 6.58 (s, 1H), 5.14 (d, J = 18.6 Hz, 3H), 4.86 (s, 1H), 4.10 (s, 3H), 3.98-3.74 (m, 2H), 3.00 (q, J = 7.4 Hz, 1H), 2.81-2.58 (m, 1H), 2.02 (d, J = 48.5 Hz, 1H), 1.10 (d, J = 6.7 Hz, 3H), 0.83 (s, 1H), 0.51-0.19 (m, 3H), 0.21- 0.01 (m, 1H). | 487.20 | +11 |
| cyclopropylethyl) | | | | |
| carbamate | | | | |
| (3R,5R)-5-(3-(1- methyl-3- ((trifluoromethoxy) methyl)-1H-pyrazole-5- carboxamido)-1H- pyrazol-5- yl)tetrahydrofuran-3-yl bicyclo[1.1.1]pentan- 1-ylcarbamate | 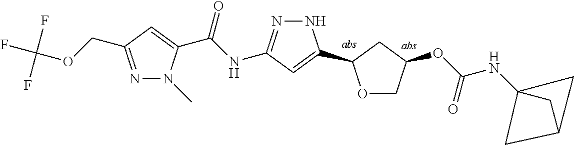 [SMILES: Cn1nc(COC(F)(F)F)cc1C(=O)Nc1cc([C@H]2C[C@@H](OC(=O)NC34CC(C3)C4)CO2)[nH]n1] | 1H NMR (400 MHz, DMSO-d6) 12.52 (s, 1H), 10.92 (s, 1H), 7.93 (s, 1H), 7.29 (s, 1H), 6.58 (s, 1H), 5.17 (dd, J = 7.5, 3.7 Hz, 1H), 5.12 (s, 2H), 4.86 (s, 1H), 4.10 (s, 3H), 3.86 (s, 2H), 2.70 (dd, J = 12.8, 5.8 Hz, 1H), 2.35 (d, J = 12.0 Hz, 1H), 1.92 (s, 7H). | 485.15 | +33 |
| (3R,5R)-5-(3-(1- methyl-3-((2,2,2- trifluoroethoxy)methyl)- 1H-pyrazole-5- carboxamido)-1H- pyrazol-5- yl)tetrahydrofuran-3-yl (1-methylcyclopropyl) | 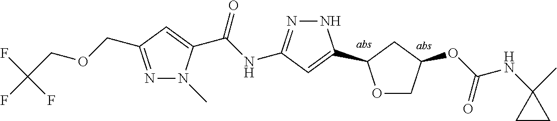 [SMILES: Cn1nc(COCC(F)(F)F)cc1C(=O)Nc1cc([C@H]2C[C@@H](OC(=O)NC3(C)CC3)CO2)[nH]n1] | 1H NMR (400 MHz, DMSO-d6) 12.47 (s, 1H), 10.86 (s, 1H), 7.52 (s, 1H), 7.17 (s, 1H), 6.52 (s, 1H), 5.16 (s, 1H), 4.84 (t, J = 7.4 Hz, 1H), 4.61 (s, 2H), 4.14-4.05 (m, 5H), 3.84 (d, J = 4.8 Hz, 2H), 2.70 (dt, J = 13.6, 7.6 Hz, 1H), 1.94 (dd, J = 14.9, 6.8 Hz, 1H), 1.25 (s, 3H), 0.61 (d, J = 5.2 Hz, 2H), 0.48 (q, J = 4.7 Hz, 2H). | 487.20 | 14 |
| carbamate | | | | |
| (3S,5S)-5-(3-(1- methyl-3- ((trifluoromethoxy) methyl)-1H-pyrazole-5- carboxamido)-1H- pyrazol-5- yl)tetrahydrofuran-3- yl (1- methylcyclopropyl) | 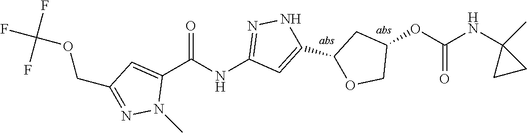 [SMILES: Cn1nc(COC(F)(F)F)cc1C(=O)Nc1cc([C@@H]2C[C@H](OC(=O)NC3(C)CC3)CO2)[nH]n1] | 1H NMR (400 MHz, DMSO-d6) 12.52 (s, 1H), 10.91 (s, 1H), 7.51 (s, 1H), 7.29 (s, 1H), 6.57 (s, 1H), 5.14 (d, J = 17.9 Hz, 3H), 4.86 (s, 1H), 4.10 (s, 3H), 3.85 (s, 2H), 2.71 (dd, J = 13.9, 7.7 Hz, 1H), 1.93 (s, 1H), 1.25 (s, 3H), 0.62 (s, 2H), 0.48 (q, J = 4.6 Hz, 2H). | 473.15 | −9 |
| carbamate | | | | |
| (3R,5R)-5-(3-((2- (methoxymethyl) pyrazolo[1,5-a]pyrazin-4- yl)amino)-1H- pyrazol-5- yl)tetrahydrofuran-3- yl ((1R,2S)-2- methylcyclopropyl) carbamate | 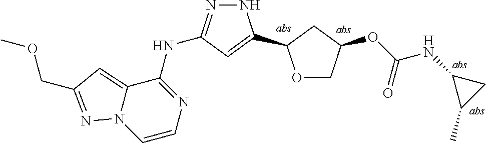 [SMILES: COCc1cc2c(Nc3cc([C@H]4C[C@@H](OC(=O)N[C@@H]5C[C@@H]5C)CO4)[nH]n3)nccn2n1] | 1H NMR (400 MHz, DMSO-d6) 12.37 (s, 1H), 10.00 (s, 1H), 8.03 (s, 1H), 7.41-7.16 (m, 3H), 6.87 (d, J = 61.3 Hz, 1H), 5.19 (s, 1H), 4.93 (d, J = 50.3 Hz, 1H), 4.57 (s, 2H), 3.88 (d, J = 17.3 Hz, 2H), 3.33 (s, 3H), 2.73 (s, 1H), 2.48-2.43 (m, 1H), 2.05 (d, J = 45.5 Hz, 1H), 0.91 (d, J = 5.9 Hz, 3H), 0.83-0.64 (m, 2H), 0.10 (d, J = 6.1 Hz, 1H). | 428.15 | |
| rel-(3R,5R)-5-(3-((2- (methoxymethyl) pyrazolo[1,5-a]pyrazin-4- yl)amino)-1H- pyrazol-5- yl)tetrahydrofuran-3- yl ((R*)-4,4,4- trifluorobutan-2- yl)carbamate | 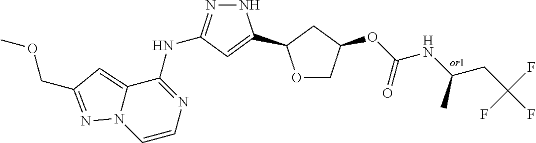 [SMILES: COCc1cc2c(Nc3cc([C@H]4C[C@@H](OC(=O)N[C@H](C)CC(F)(F)F)CO4)[nH]n3)nccn2n1] | 1H NMR (400 MHz, DMSO-d6) 12.40 (s, 1H), 10.03 (s, 1H), 8.04 (s, 1H), 7.38 (d, J = 7.6 Hz, 2H), 7.29 (s, 1H), 6.80 (s, 1H), 5.20 (s, 1H), 4.85 (s, 1H), 4.57 (s, 2H), 3.86 (dd, J = 13.5, 7.8 Hz, 3H), 3.33 (s, 3H), 2.74 (s, 1H), 2.38 (dd, J = 12.7, 4.5 Hz, 2H), 2.01 (d, J = 36.4 Hz, 1H), 1.14 (d, J = 6.7 Hz, 3H). | 484.15 | 5 |
| (3R,5R)-5-(3-((2- (methoxymethyl) pyrazolo[1,5-a]pyrazin-4- yl)amino)-1H- pyrazol-5- yl)tetrahydrofuran-3- yl ((S)-1,1- difluoropropan-2- yl)carbamate | 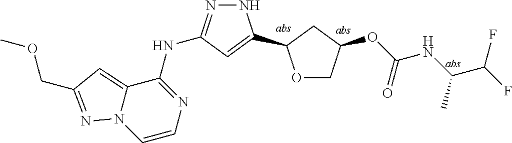 [SMILES: COCc1cc2c(Nc3cc([C@H]4C[C@@H](OC(=O)N[C@@H](C)C(F)F)CO4)[nH]n3)nccn2n1] | 1H NMR (400 MHz, DMSO-d6) 12.37 (s, 1H), 10.02 (s, 1H), 8.04 (s, 1H), 7.61 (d, J = 8.5 Hz, 1H), 7.39 (d, J = 4.8 Hz, 1H), 7.29 (s, 1H), 6.81 (s, 1H), 5.90 (t, J = 55.9 Hz, 1H), 5.23 (s, 1H), 4.86 (s, 1H), 4.58 (s, 2H), 4.05-3.71 (m, 3H), 3.34 (s, 3H), 2.75 (s, 1H), 2.00 (s, 1H), 1.11 (d, J = 7.0 Hz, 3H). | 452.15 | +27 |
| (3R,5R)-5-(3-((2- (methoxymethyl) pyrazolo[1,5-a]pyrazin-4- yl)amino)-1H- pyrazol-5- yl)tetrahydrofuran-3- yl ((R)-1,1- difluoropropan-2- yl)carbamate | 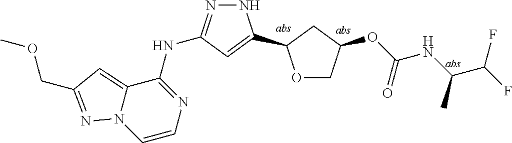 [SMILES: COCc1cc2c(Nc3cc([C@H]4C[C@@H](OC(=O)N[C@H](C)C(F)F)CO4)[nH]n3)nccn2n1] | 1H NMR (400 MHz, DMSO-d6) 12.37 (s, 1H), 10.04 (s, 1H), 8.05 (s, 1H), 7.60 (d, J = 8.5 Hz, 1H), 7.40 (d, J = 4.8 Hz, 1H), 7.27 (s, 1H), 6.79 (s, 1H), 6.14-5.67 (m, 1H), 5.21 (d, J = 7.5 Hz, 1H), 4.86 (s, 1H), 4.58 (s, 2H), 3.96-3.84 (m, 3H), 3.34 (s, 3H), 2.74 (s, 1H), 2.01 (s, 1H), 1.14-1.07 (m, 3H). | 452.15 | +19 |
| (3R,5R)-5-(3-(3- ((difluoromethoxy) methyl)-1-methyl-1H- pyrazole-5- carboxamido)-1H- pyrazol-5- yl)tetrahydrofuran-3- yl ((S)-1- cyclopropylethyl) carbamate | 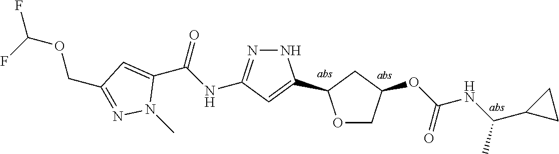 [SMILES: C[C@H](NC(=O)O[C@H]1CO[C@@H](c2cc(NC(=O)c3cc(COC(F)F)nn3C)n[nH]2)C1)C1CC1] | 1H NMR (400 MHz, DMSO-d6) 12.51 (s, 1H), 10.86 (s, 1H), 7.32-7.08 (m, 2H), 6.68 (d, J = 76.1 Hz, 2H), 5.17 (s, 1H), 4.86 (s, 3H), 4.08 (s, 3H), 3.87 (s, 2H), 3.00 (s, 1H), 1.96 (s, 1H), 2.80-2.62(s.1H), 1.11 (d, J = 6.6 Hz, 3H), 0.83 (s, 1H), 0.48-0.24 (m, 3H), 0.11 (s, 1H). | 469.20 | |
| (3R,5R)-5-(3-(1- methyl-3- (trifluoromethyl)-1H- pyrazole-5- carboxamido)-1H- pyrazol-5- yl)tetrahydrofuran-3- yl (1- | 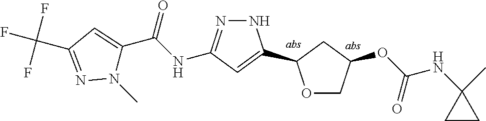 [SMILES: Cn1nc(C(F)(F)F)cc1C(=O)Nc1cc([C@H]2C[C@@H](OC(=O)NC3(C)CC3)CO2)[nH]n1] | 1H NMR (400 MHz, DMSO-d6) 12.57 (s, 1H), 11.07 (s, 1H), 7.57 (d, J = 36.6 Hz, 2H), 6.58 (s, 1H), 5.16 (s, 1H), 4.86 (s, 1H), 4.18 (s, 3H), 3.85 (s, 2H), 2.70 (s, 1H), 1.93 (s, 1H), 1.25 (s, 3H), 0.55 (d, J = 53.6 Hz, 4H). | 443.1 | +30 |
| methylcyclopropyl) | | | | |
| carbamate | | | | |
| (3R,5R)-5-(3- (pyrazolo[1,5- a]pyrazin-4-ylamino)- 1H-pyrazol-5- yl)tetrahydrofuran-3- yl (1- methylcyclopropyl) carbamate | 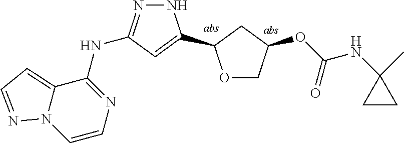 [SMILES: CC1(NC(=O)O[C@H]2CO[C@@H](c3cc(Nc4nccn5nccc45)n[nH]3)C2)CC1] | 1H NMR (400 MHz, DMSO-d6) 12.37 (s, 1H), 9.96 (d, J = 69.1 Hz, 1H), 8.10 (d, J = 4.8 Hz, 1H), 7.98 (d, J = 2.3 Hz, 1H), 7.53 (s, 1H), 7.48-7.09 (m, 2H), 6.37 (d, J = 263.6 Hz, 1H), 5.17 (s, 1H), 4.83 (s, 1H), 3.85 (d, J = 6.2 Hz, 2H), 2.71 (dt, J = 13.3, 7.0 Hz, 1H), 1.96 (s, 1H), 1.25 (s, 3H), 0.61 (s, 2H), 0.48 (q, J = 4.9, 4.5 Hz, 2H). | 384.10 | +20 |
| (3R,5R)-5-(3-(1- methyl-3-(2,2,2- trifluoroethoxy)-1H- pyrazole-5- carboxamido)-1H- pyrazol-5- yl)tetrahydrofuran-3- yl (1- methylcyclopropyl) carbamate | 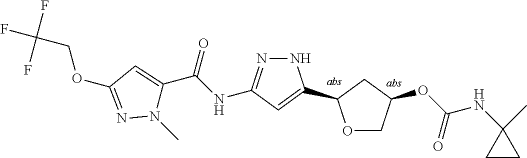 [SMILES: Cn1nc(OCC(F)(F)F)cc1C(=O)Nc1cc([C@H]2C[C@@H](OC(=O)NC3(C)CC3)CO2)[nH]n1] | 1H NMR (400 MHz, DMSO-d6) 12.53 (s, 1H), 10.82 (s, 1H), 7.50 (s, 1H), 6.62 (d, J = 48.9 Hz, 2H), 5.16 (s, 1H), 4.80 (q, J = 8.9 Hz, 3H), 3.97 (s, 3H), 3.85 (s, 2H), 2.70 (dt, J = 13.1, 6.9 Hz, 1H), 1.93 (s, 1H), 1.25 (s, 3H), 0.65-0.37 (m, 4H). | 473.05 | +22 |
| (3S,5S)-5-(3-(1- methyl-3- ((trifluoromethoxy) methyl)-1H-pyrazole-5- carboxamido)-1H- pyrazol-5- yl)tetrahydrofuran-3- yl bicyclo[1.1.1]pentan- 1-ylcarbamate | 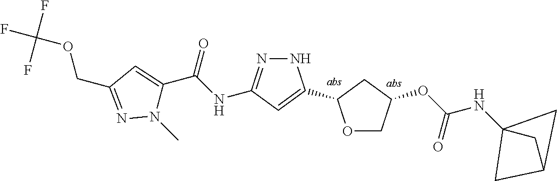 [SMILES: Cn1nc(COC(F)(F)F)cc1C(=O)Nc1cc([C@@H]2C[C@H](OC(=O)NC34CC(C3)C4)CO2)[nH]n1] | 1H NMR (400 MHz, DMSO-d6) 12.52 (s, 1H), 10.91 (s, 1H), 7.92 (s, 1H), 7.29 (s, 1H), 6.58 (s, 1H), 5.17 (d, J = 3.7 Hz, 1H), 5.12 (s, 2H), 4.87 (s, 1H), 4.10 (s, 3H), 3.86 (s, 2H), 2.70 (d, J = 8.2 Hz, 1H), 2.36 (s, 1H), 2.00 (m, 7H). | 485.10 | −10 |
| (3S,5S)-5-(3-(1- methyl-3- ((trifluoromethoxy) methyl)-1H-pyrazole-5- carboxamido)-1H- pyrazol-5- yl)tetrahydrofuran-3- yl ((S)-1- cyclopropylethyl) carbamate | 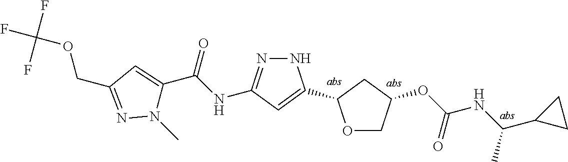 [SMILES: C[C@H](NC(=O)O[C@@H]1CO[C@H](c2cc(NC(=O)c3cc(COC(F)(F)F)nn3C)n[nH]2)C1)C1CC1] | 1H NMR (400 MHz, DMSO-d6) 12.45 (s, 1H), 10.92 (s, 1H), 7.29 (s, 1H), 7.17 (d, J = 8.4 Hz, 1H), 6.55 (s, 1H), 5.17 (s, 1H), 5.12 (s, 2H), 4.85 (t, J = 7.6 Hz, 1H), 4.10 (s, 3H), 3.86 (m, 2H), 3.00 (q, J = 7.1 Hz, 1H), 2.72 (p, J = 7.2 Hz, 1H), 1.96 (t, J = 10.2 Hz, 1H), 1.10 (d, J = 6.7 Hz, 3H), 1.02 (s, 1H), 0.83 (s, 1H), 0.38 (s, 1H), 0.41-0.21 (m, 1H), 0.11 (dd, J = 8.9, 4.4 Hz, 1H). | 487.15 | −8 |
| (3R,5R)-5-(3-(3-(2,2- difluoroethoxy)-1- methyl-1H-pyrazole- 5-carboxamido)-1H- pyrazol-5- yl)tetrahydrofuran-3- yl (1- methylcyclopropyl) carbamate | 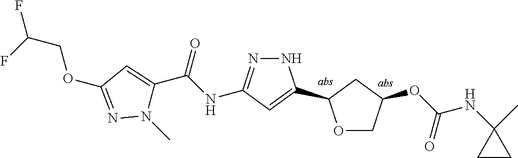 [SMILES: Cn1nc(OCC(F)F)cc1C(=O)Nc1cc([C@H]2C[C@@H](OC(=O)NC3(C)CC3)CO2)[nH]n1] | 1H NMR (400 MHz, DMSO-d6) 12.53 (s, 1H), 10.79 (s, 1H), 7.50 (s, 1H), 6.65 (s, 1H), 6.60- 6.20 (m, 2H), 5.16 (s, 1H), 4.85 (s, 1H), 4.38 (td, J = 14.9, 3.5 Hz, 2H), 3.90 (d, J = 45.2 Hz, 5H), 2.71 (dd, J = 14.2, 7.6 Hz, 1H), 1.92 (s, 1H), 1.25 (s, 3H), 0.62 (s, 2H), 0.49 (d, J = 5.4 Hz, 2H). | 455.15 | +58 |
| (3R,5R)-5-(3-((2- (methoxymethyl) pyrazolo[1,5-a]pyrazin-4- yl)amino)-1H- pyrazol-5- yl)tetrahydrofuran-3- yl ((1S,2R)-2- methylcyclopropyl) carbamate | 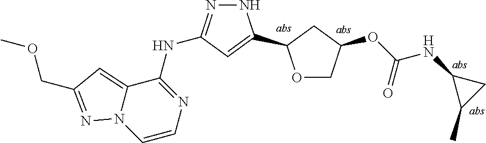 [SMILES: COCc1cc2c(Nc3cc([C@H]4C[C@@H](OC(=O)N[C@H]5C[C@H]5C)CO4)[nH]n3)nccn2n1] | 1H NMR (400 MHz, DMSO-d6) 12.38 (s, 1H), 10.04 (s, 1H), 8.04 (s, 1H), 7.44-7.21 (m, 3H), 6.88 (d, J = 56.0 Hz, 1H), 5.21 (s, 1H), 4.85 (s, 1H), 4.57 (s, 2H), 3.87 (s, 2H), 3.33 (s, 3H), 2.72 (s, 1H), 2.47 (s, 1H), 2.01 (s, 1H), 0.93 (d, J = 5.9 Hz, 3H), 0.85-0.62 (m, 2H), 0.12 (d, J = 4.7 Hz, 1H). | 428.20 | 12 |
| (3S,5S)-5-(3-(3-(2,2- difluoroethoxy)-1- methyl-1H-pyrazole- 5-carboxamido)-1H- pyrazol-5- yl)tetrahydrofuran-3- yl bicyclo[1.1.1]pentan- 1-ylcarbamate | 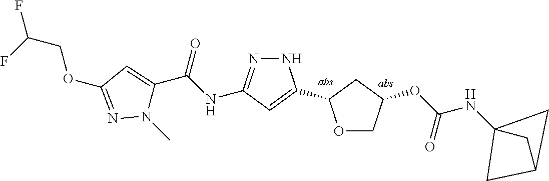 [SMILES: Cn1nc(OCC(F)F)cc1C(=O)Nc1cc([C@@H]2C[C@H](OC(=O)NC34CC(C3)C4)CO2)[nH]n1] | 1H NMR (400 MHz, DMSO-d6) 12.55 (s, 1H), 10.80 (s, 1H), 7.94 (s, 1H), 6.69-6.49 (m, 2H), 6.38 (t, J = 3.5 Hz, 1H), 5.16 (d, J = 3.4 Hz, 1H), 4.86 (s, 1H), 4.38 (td, J = 14.9, 3.5 Hz, 2H), 3.96 (s, 3H), 3.86 (s, 2H), 2.70 (d, J = 14.8 Hz, 1H), 2.37 (s, 1H), 1.92 (s, 7H). | 467.15 | |
| (3S,5S)-5-(3-(1- methyl-3-(2,2,2- trifluoroethoxy)-1H- pyrazole-5- carboxamido)-1H- pyrazol-5- yl)tetrahydrofuran-3- yl bicyclo[1.1.1]pentan- 1-ylcarbamate | 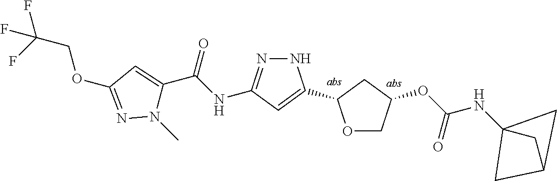 [SMILES: Cn1nc(OCC(F)(F)F)cc1C(=O)Nc1cc([C@@H]2C[C@H](OC(=O)NC34CC(C3)C4)CO2)[nH]n1] | 1H NMR (400 MHz, DMSO-d6) 12.55 (s, 1H), 10.83 (s, 1H), 7.92 (s, 1H), 6.63 (d, J = 44.6 Hz, 2H), 5.17 (dd, J = 7.4, 3.8 Hz, 1H), 4.81 (q, J = 14.4, 8.9 Hz, 3H), 4.31-3.68 (m, 5H), 2.71 (q, J = 7.5 Hz, 1H), 2.36 (s, 1H), 2.07-1.69 (m, 7H) | 484.15 | |
| (3R,5R)-5-(3-(3-(2,2- difluoroethoxy)-1- methyl-1H-pyrazole- 5-carboxamido)-1H- pyrazol-5- yl)tetrahydrofuran-3- yl bicyclo[1.1.1]pentan- 1-ylcarbamate | 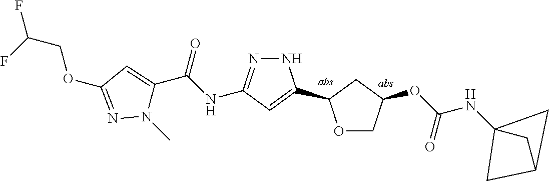 [SMILES: Cn1nc(OCC(F)F)cc1C(=O)Nc1cc([C@H]2C[C@@H](OC(=O)NC34CC(C3)C4)CO2)[nH]n1] | 1H NMR (400 MHz, DMSO-d6) 12.53 (s, 1H), 10.63 (s, 1H), 7.91 (s, 1H), 6.68-6.47 (m, 2H), 6.38 (t, J = 3.5 Hz, 1H), 5.17 (dt, J = 7.3, 3.5 Hz, 1H), 4.86 (s, 1H), 4.38 (td, J = 14.9, 3.5 Hz, 2H), 3.96 (s, 3H), 3.86 (s, 2H), 2.70 (dt, J = 13.6, 7.6 Hz, 1H), 2.36 (s, 1H), 1.92 (s, 7H). | 467.20 | |
| (3R,5R)-5-(3-(1- methyl-3-(2,2,2- trifluoroethoxy)-1H- pyrazole-5- carboxamido)-1H- pyrazol-5- yl)tetrahydrofuran-3-yl bicyclo[1.1.1]pentan- 1-ylcarbamate | 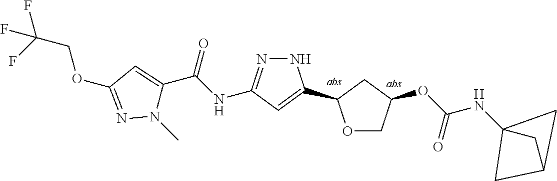 [SMILES: Cn1nc(OCC(F)(F)F)cc1C(=O)Nc1cc([C@H]2C[C@@H](OC(=O)NC34CC(C3)C4)CO2)[nH]n1] | 1H NMR (400 MHz, DMSO-d6) 12.54 (s, 1H), 10.83 (s, 1H), 7.92 (s, 1H), 6.63 (d, J = 45.6 Hz, 2H), 5.16 (t, J = 4.1 Hz, 1H), 4.80 (q, J = 8.9 Hz, 3H), 3.97 (s, 3H), 3.86 (s, 2H), 2.71 (dd, J = 14.4, 7.7 Hz, 1H), 2.35 (d, J = 11.4 Hz, 1H), 1.92 (s, 7H). | 485.15 | |
| (3R,5R)-5-(3-(1- methyl-3- ((trifluoromethoxy) methyl)-1H-pyrazole-5- carboxamido)-1H- pyrazol-5- yl)tetrahydrofuran-3- yl (2-cyanopropan-2- yl)carbamate | 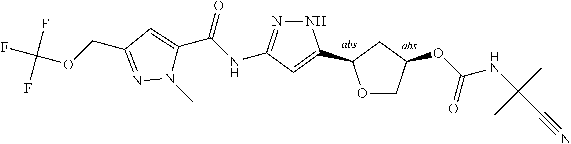 [SMILES: Cn1nc(COC(F)(F)F)cc1C(=O)Nc1cc([C@H]2C[C@@H](OC(=O)NC(C)(C)C#N)CO2)[nH]n1] | 1H NMR (400 MHz, DMSO-d6) 12.56 (s, 1H), 10.93 (s, 1H), 8.09 (s, 1H), 7.29 (s, 1H), 6.58 (s, 1H), 5.26 (dp, J = 7.6, 2.4 Hz, 1H), 5.12 (s, 2H), 4.87 (s, 1H), 4.10 (s, 3H), 4.00-3.74 (m, 2H), 2.77 (dt, J = 12.8, 6.7 Hz, 1H), 2.00 (d, J = 12.2 Hz, 1H), 1.56 (d, J = 4.2 Hz, 6H). | 486.15 | |
| (3R,5R)-5-(3-(3- (methoxymethyl)-1- methyl-1H-pyrazole- 5-carboxamido)-1H- pyrazol-5- yl)tetrahydrofuran-3- yl (1-methylcyclopropyl) carbamate | 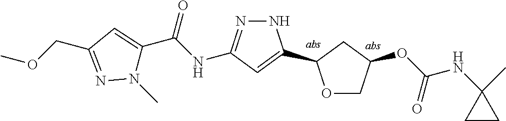 | 1H NMR (400 MHz, DMSO-d6) 12.50 (s, 1H), 10.80 (s, 1H), 7.52 (s, 1H), 7.13 (s, 1H), 6.56 (s, 1H), 5.16 (s, 1H), 4.85 (s, 1H), 4.35 (s, 2H), 4.06 (s, 3H), 3.85 (s, 2H), 3.28 (s, 3H), 2.70 (s, 1H), 1.93 (s, 1H), 1.25 (s, 3H), 0.62 (s, 2H), 0.48 (s, 2H). | 419.10 | |
| (3R,5R)-5-(3-(1- methyl-3- ((trifluoromethoxy) methyl)-1H-pyrazole-5- carboxamido)-1H- pyrazol-5- yl)tetrahydrofuran-3- yl cuban-1- | 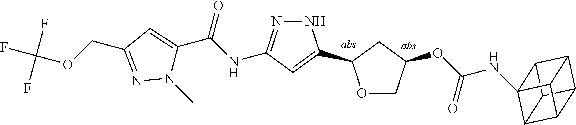 [SMILES: Cn1nc(COC(F)(F)F)cc1C(=O)Nc1cc([C@H]2C[C@@H](OC(=O)NC34C5C6C7C5C3C7C64)CO2)[nH]n1] | 1H NMR (400 MHz, DMSO-d6) 12.52 (s, 1H), 10.91 (s, 1H), 7.90 (d, J = 105.4 Hz, 1H), 7.29 (s, 1H), 6.58 (s, 1H), 5.15 (d, J = 28.2 Hz, 3H), 4.88 (s, 1H), 4.10 (s, 3H), 4.03 (s, 2H), 3.97- 3.78 (m, 6H), 3.74 (s, 1H), 2.72 (t, J = 7.2 Hz, 1H), 2.03 (d, J = 39.4 Hz, 1H). | 521.10 | |
| ylcarbamate | | | | |
| (3R,5R)-5-(3-(1- methyl-3- ((trifluoromethoxy) methyl)-1H-pyrazole-5- carboxamido)-1H- pyrazol-5- yl)tetrahydrofuran-3- yl (1- | 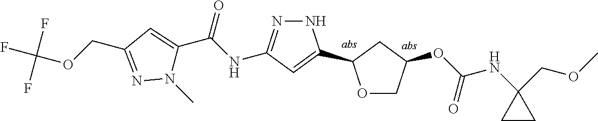 [SMILES: COCC1(NC(=O)O[C@H]2CO[C@@H](c3cc(NC(=O)c4cc(COC(F)(F)F)nn4C)n[nH]3)C2)CC1] | 1H NMR (400 MHz, DMSO-d6) 12.50 (s, 1H), 10.91 (s, 1H), 7.59 (s, 1H), 7.29 (s, 1H), 6.57 (s, 1H), 5.14 (d, J = 19.2 Hz, 3H), 4.86 (s, 1H), 4.10 (s, 3H), 3.85 (s, 2H), 3.29 (s, 2H), 3.24 (s, 3H), 2.70 (s, 1H), 1.93 (s, 1H), 0.65 (d, J = 4.7 Hz, 4H). | 503.10 | |
| (methoxymethyl)cyclo- | | | | |
| propyl)carbamate | | | | |
| (3R,5R)-5-(3-(1- methyl-3- ((trifluoromethoxy) methyl)-1H-pyrazole-5- carboxamido)-1H- pyrazol-5- yl)tetrahydrofuran-3- yl (3-methyloxetan-3- yl)carbamate | 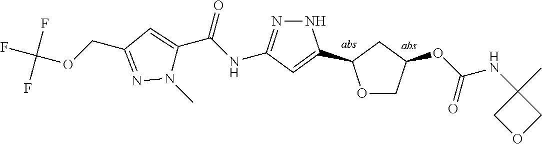 [SMILES: Cn1nc(COC(F)(F)F)cc1C(=O)Nc1cc([C@H]2C[C@@H](OC(=O)NC3(C)COC3)CO2)[nH]n1] | 1H NMR (400 MHz, DMSO-d6) 12.53 (s, 1H), 10.91 (s, 1H), 7.78 (s, 1H), 7.29 (s, 1H), 6.58 (s, 1H), 5.20 (t, J = 4.4 Hz, 1H), 5.12 (s, 2H), 4.87 (s, 1H), 4.59 (s, 2H), 4.25 (s, 2H), 4.10 (s, 3H), 3.90 (d, J = 11.1 Hz, 2H), 2.70 (d, J = 15.0 Hz, 1H), 2.04 (d, J = 34.3 Hz, 1H), 1.49 (s, 3H). | 489.15 | |
| (3S,5S)-5-(3-(1- methyl-3- ((trifluoromethoxy) methyl)-1H-pyrazole-5- carboxamido)-1H- pyrazol-5- yl)tetrahydrofuran-3- yl (3-methyloxetan-3- yl)carbamate | 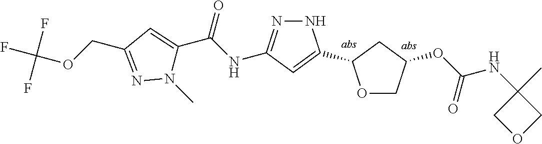 [SMILES: Cn1nc(COC(F)(F)F)cc1C(=O)Nc1cc([C@@H]2C[C@H](OC(=O)NC3(C)COC3)CO2)[nH]n1] | 1H NMR (400 MHz, DMSO-d6) 12.55 (s, 1H), 10.93 (s, 1H), 7.79 (s, 1H), 7.29 (s, 1H), 6.59 (s, 1H), 5.19 (dq, J = 7.2, 3.2 Hz, 1H), 5.12 (s, 2H), 4.87 (s, 1H), 4.59 (d, J = 6.0 Hz, 2H), 4.25 (d, J = 6.0 Hz, 2H), 4.10 (s, 3H), 3.95-3.78 (m, 2H), 2.72 (s, 1H), 2.00 (d, J = 10.8 Hz, 1H), 1.49 (s, 3H). | 489.20 | |
| (3S,5S)-5-(3-(1- methyl-3- ((trifluoromethoxy) methyl)-1H-pyrazole-5- carboxamido)-1H- pyrazol-5- yl)tetrahydrofuran-3- yl (1- | 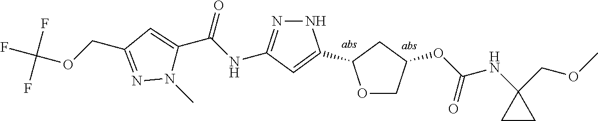 [SMILES: COCC1(NC(=O)O[C@@H]2CO[C@H](c3cc(NC(=O)c4cc(COC(F)(F)F)nn4C)n[nH]3)C2)CC1] | 1H NMR (400 MHz, DMSO-d6) 12.50 (s, 1H), 10.92 (s, 1H), 7.59 (s, 1H), 7.29 (s, 1H), 6.57 (s, 1H), 5.12 (s, 4H), 4.10 (s, 3H), 3.85 (s, 2H), 3.30 (d, J = 7.1 Hz, 2H), 3.24 (s, 3H), 2.69 (d, J = 6.7 Hz, 1H), 1.93 (s, 1H), 0.65 (d, J = 4.5 Hz, 4H). | 503.20 | |
| (methoxymethyl) | | | | |
| cyclopropyl)carbamate | | | | |
| N-(5-((2R,4R)-4-((4- isopropyl-1H- pyrazol-5- yl)oxy)tetrahydrofuran- 2-yl)-1H-pyrazol-3- yl)-3- (methoxymethyl)-1- methyl-1H-pyrazole- | 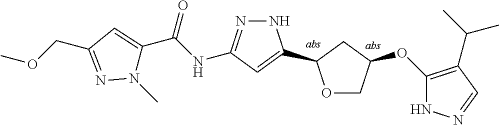 [SMILES: COCc1cc(C(=O)Nc2cc([C@H]3C[C@@H](Oc4[nH]ncc4C(C)C)CO3)[nH]n2)n(C)n1] | 1H NMR (400 MHz, dmso) 12.49 (br. s, 1H), 11.54 (s, 1H), 10.78 (br. s, 1H), 7.26 (s, 1H), 7.11 (s, 1H), 6.58 (br. s, 1H), 5.25-5.17 (m, 1H), 5.02-4.88 (m, 1H), 4.34 (s, 2H), 4.05 (s, 3H), 4.04-3.91 (m, 2H), 3.27 (submerged, s, 3H), 2.79-2.62 (m, 2H), 2.14 (ddd, J = 13.4, 6.5, 2.8 Hz, 1H), 1.08 (d, J = 6.8 Hz, 6H). | 430.30 | |
| 5-carboxamide | | | | |
| (3R,5R)-5-(3-(3- (hydroxymethyl)-1- methyl-1H-pyrazole- 5-carboxamido)-1H- pyrazol-5- yl)tetrahydrofuran-3- yl (1-methylcyclopropyl) carbamate | 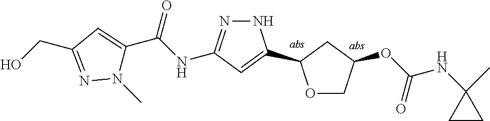 [SMILES: Cn1nc(CO)cc1C(=O)Nc1cc([C@H]2C[C@@H](OC(=O)NC3(C)CC3)CO2)[nH]n1] | 1H NMR (400 MHz, DMSO-d6) 12.48 (s, 1H), 10.76 (s, 1H), 7.51 (s, 1H), 7.09 (s, 1H), 6.56 (s, 1H), 5.14 (d, J = 19.0 Hz, 2H), 4.85 (s, 1H), 4.41 (d, J = 5.7 Hz, 2H), 4.04 (s, 3H), 3.85 (s, 2H), 2.71 (d, J = 7.1 Hz, 1H), 2.01 (d, J = 59.5 Hz, 1H), 1.25 (s, 3H), 0.62 (s, 2H), 0.48 (q, J = 4.6 Hz, 2H). | 405.05 | |
| rel-(3R,5R)-5-(2-(1- methyl-3- ((trifluoromethoxy) methyl)-1H-pyrazole-5- carboxamido)thiazol- 5-yl)tetrahydrofuran- 3-yl (1- methylcyclopropyl) | 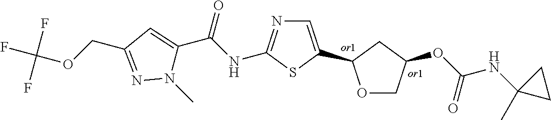 [SMILES: Cn1nc(COC(F)(F)F)cc1C(=O)Nc1ncc([C@H]2C[C@@H](OC(=O)NC3(C)CC3)CO2)s1] | 1H NMR (400 MHz, DMSO-d6) 12.75 (s, 1H), 7.56 (s, 1H), 7.47 (s, 1H), 7.38 (s, 1H), 5.14 (s, 4H), 4.14 (s, 3H), 3.85 (dd, J = 19.4, 7.9 Hz, 2H), 2.76 (dd, J = 14.1, 7.2 Hz, 1H), 1.89 (s, 1H), 1.24 (d, J = 6.7 Hz, 3H), 0.63 (s, 2H), 0.49 (s, 2H). | 490.15 | |
| carbamate | | | | |
| (3R,5R)-5-(3-(3-(2,2- difluoroethoxy)-1- methyl-1H-pyrazole- 5-carboxamido)-1H- pyrazol-5- yl)tetrahydrofuran-3- yl ((R)-1- cyclopropylethyl) | 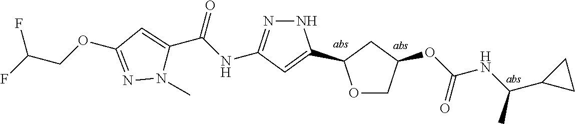 [SMILES: C[C@@H](NC(=O)O[C@H]1CO[C@@H](c2cc(NC(=O)c3cc(OCC(F)F)nn3C)n[nH]2)C1)C1CC1] | 1H NMR (400 MHz, DMSO-d6) 12.53 (s, 1H), 10.76 (d, J = 4.6 Hz, 1H), 7.17 (d, J = 8.5 Hz, 1H), 6.64 (s, 1H), 6.54 (s, 1H), 6.38 (tt, J = 54.5, 3.4 Hz, 1H), 5.16 (s, 1H), 4.85 (t, J = 7.7 Hz, 1H), 4.38 (td, J = 14.9, 3.5 Hz, 2H), 3.96 (s, 3H), 3.86 (d, J = 9.4 Hz, 2H), 2.99 (d, J = 7.4 Hz, 1H), 2.77-2.66 (m, 1H), 1.95 (s, 1H), 1.10 (d, J = 6.7 Hz, 3H), 0.82 (d, J = 5.6 Hz, 1H), | 469.10 | |
| carbamate | | 0.45-0.21 (m, 3H), 0.10 (d, J = 4.5 Hz, 1H). | | |
| (3R,5R)-5-(3-(3-(2,2- difluoroethoxy)-1- methyl-1H-pyrazole- 5-carboxamido)-1H- pyrazol-5- yl)tetrahydrofuran-3- yl ((S)-1- cyclopropylethyl) | 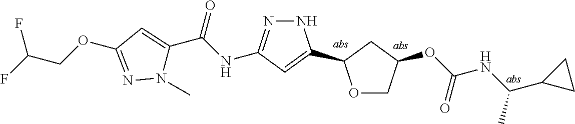 [SMILES: C[C@H](NC(=O)O[C@H]1CO[C@@H](c2cc(NC(=O)c3cc(OCC(F)F)nn3C)n[nH]2)C1)C1CC1] | 1H NMR (400 MHz, DMSO-d6) 12.59-12.54 (m, 1H), 10.81 (s, 1H), 7.18 (d, J = 8.4 Hz, 1H), 6.66 (s, 1H), 6.58 (s, 1H), 6.38 (tt, J = 54.5, 3.4 Hz, 1H), 5.16 (s, 1H), 4.86 (t, J = 7.7 Hz, 1H), 4.38 (td, J = 14.9, 3.5 Hz, 2H), 3.96 (s, 3H), 3.87 (q, J = 6.5, 4.9 Hz, 2H), 3.00 (q, J = 7.5 Hz, 1H), 2.77-2.67 (m, 1H), 1.96 (d, J = 11.6 Hz, 1H), 1.10 (d, J = 6.7 Hz, 3H), 0.82 (dd, J = | 469.10 | |
| carbamate | | 8.2, 4.6 Hz, 1H), 0.42-0.21 (m, 3H), 0.11 (dd, | | |
| | | J = 9.4, 4.7 Hz, 1H). | | |
| (3R,5R)-5-(3-(1- methyl-3-(2,2,2- trifluoroethoxy)-1H- pyrazole-5- carboxamido)-1H- pyrazol-5- yl)tetrahydrofuran-3- yl ((R)-1- | 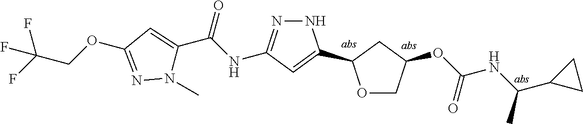 [SMILES: C[C@@H](NC(=O)O[C@H]1CO[C@@H](c2cc(NC(=O)c3cc(OCC(F)(F)F)nn3C)n[nH]2)C1)C1CC1] | 1H NMR (400 MHz, DMSO-d6) 12.56 (s, 1H), 10.84 (s, 1H), 7.18 (d, J = 8.5 Hz, 1H), 6.69 (s, 1H), 6.58 (s, 1H), 5.17 (s, 1H), 4.80 (q, J = 8.9 Hz, 3H), 3.97 (s, 3H), 3.86 (s, 2H), 3.00 (d, J = 7.1 Hz, 1H), 2.72 (s, 1H), 1.96 (s, 1H), 1.10 (d, J = 6.7 Hz, 3H), 0.82 (s, 1H), 0.43-0.20 (m, 3H), 0.12 (d, J = 7.3 Hz, 1H). | 487.15 | |
| cyclopropylethyl) | | | | |
| carbamate | | | | |
| (3R,5R)-5-(3-(1- methyl-3-(2,2,2- trifluoroethoxy)-1H- pyrazole-5- carboxamido)-1H- pyrazol-5- yl)tetrahydrofuran-3- yl ((S)-1- | 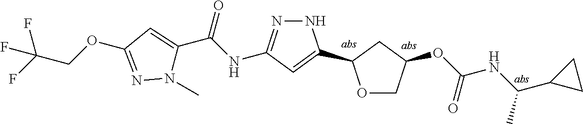 [SMILES: C[C@H](NC(=O)O[C@H]1CO[C@@H](c2cc(NC(=O)c3cc(OCC(F)(F)F)nn3C)n[nH]2)C1)C1CC1] | 1H NMR (400 MHz, DMSO-d6) 12.57 (s, 1H), 10.85 (s, 1H), 7.18 (d, J = 8.5 Hz, 1H), 6.69 (s, 1H), 6.58 (s, 1H), 5.16 (s, 1H), 4.81 (p, J = 9.0 Hz, 3H), 3.97 (s, 3H), 3.89-3.84 (m, 2H), 3.07- 2.64 (m, 2H), 1.95 (s, 1H), 1.10 (d, J = 6.7 Hz, 3H), 0.82 (d, J = 7.0 Hz, 1H), 0.54-0.15 (m, 3H), 0.15-0.08 (m, 1H). | 487.20 | |
| cyclopropylethyl) | | | | |
| carbamate | | | | |
| (3S,5S)-5-(3-(1- methyl-3- ((trifluoromethoxy) methyl)-1H-pyrazole-5- carboxamido)-1H- pyrazol-5- yl)tetrahydrofuran-3- yl (3- methoxybicyclo | 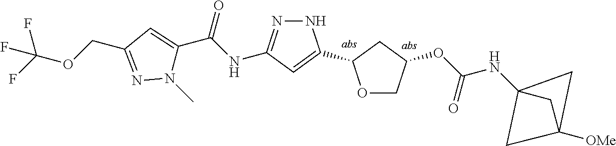 [SMILES: COC12CC(NC(=O)O[C@@H]3CO[C@H](c4cc(NC(=O)c5cc(COC(F)(F)F)nn5C)n[nH]4)C3)(C1)C2] | 1H NMR (400 MHz, DMSO-d6) 12.53 (s, 1H), 10.92 (s, 1H), 7.99 (s, 1H), 7.30 (s, 1H), 6.58 (s, 1H), 5.18 (dd, J = 7.4, 3.7 Hz, 1H), 5.12 (s, 2H), 4.87 (s, 1H), 4.10 (s, 3H), 3.87 (s, 2H), 3.18 (s, 3H), 2.70 (dd, J = 13.9, 7.2 Hz, 1H), 2.14-1.98 (m, 6H), 1.96 (s, 1H). | 515.15 | |
| [1.1.1]pentan-1- | | | | |
| yl)carbamate | | | | |
| (3S,5S)-5-(3-(1- methyl-3- ((trifluoromethoxy) methyl)-1H-pyrazole-5- carboxamido)-1H- pyrazol-5- yl)tetrahydrofuran-3- yl (3- cyanobicyclo[1.1.1] | 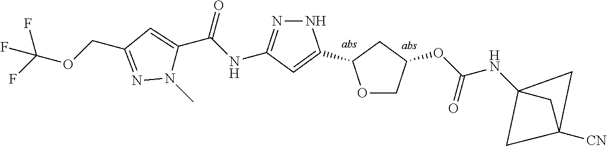 [SMILES: Cn1nc(COC(F)(F)F)cc1C(=O)Nc1cc([C@@H]2C[C@H](OC(=O)NC34CC(C#N)(C3)C4)CO2)[nH]n1] | 1H NMR (400 MHz, DMSO-d6) 12.53 (s, 1H), 10.92 (s, 1H), 8.25 (s, 1H), 7.29 (s, 1H), 6.57 (s, 1H), 5.18 (s, 1H), 5.11 (s, 2H), 4.86 (s, 1H), 4.10 (s, 3H), 3.86 (s, 2H), 2.69-2.65 (m, 1H), 2.44-2.18 (m, 6H), 1.94 (s, 1H). | 510.05 | |
| pentan-1-yl)carbamate | | | | |
| (3R,5R)-5-(3-(1- methyl-3- ((trifluoromethoxy) methyl)-1H-pyrazole-5- carboxamido)-1H- pyrazol-5- yl)tetrahydrofuran-3- yl (3-methoxybicyclo [1.1.1]pentan-1- | 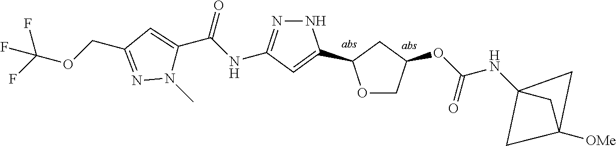 [SMILES: COC12CC(NC(=O)O[C@H]3CO[C@@H](c4cc(NC(=O)c5cc(COC(F)(F)F)nn5C)n[nH]4)C3)(C1)C2] | 1H NMR (400 MHz, DMSO-d6) 12.54 (s, 1H), 10.91 (s, 1H), 7.84 (d, J = 127.7 Hz, 1H), 7.29 (s, 1H), 6.55 (s, 1H), 5.18 (dt, J = 7.2, 3.6 Hz, 1H), 5.12 (s, 2H), 4.92 (d, J = 47.5 Hz, 1H), 4.10 (s, 3H), 3.86 (s, 2H), 3.18 (s, 3H), 2.77- 2.60 (m, 1H), 2.11-1.72 (m, 7H). | 515.15 | |
| yl)carbamate | | | | |
| (3R,5R)-5-(3-(1- methyl-3- ((trifluoromethoxy) methyl)-1H-pyrazole-5- carboxamido)-1H- pyrazol-5- yl)tetrahydrofuran-3- yl (3- cyanobicyclo[1.1.1] | 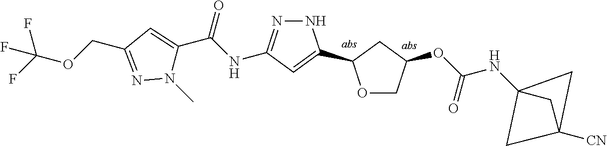 [SMILES: Cn1nc(COC(F)(F)F)cc1C(=O)Nc1cc([C@H]2C[C@@H](OC(=O)NC34CC(C#N)(C3)C4)CO2)[nH]n1] | 1H NMR (400 MHz, DMSO-d6) 12.54 (s, 1H), 10.92 (s, 1H), 8.25 (s, 1H), 7.29 (s, 1H), 6.57 (s, 1H), 5.18 (s, 1H), 5.11 (s, 2H), 4.86 (s, 1H), 4.10 (s, 3H), 3.86 (s, 2H), 2.67 (dd, J = 3.7, 1.9 Hz, 1H), 2.41 (s, 4H), 2.35-2.13 (m, 2H), 1.94 (s, 1H) | 510.30 | |
| pentan-1-yl)carbamate | | | | |

### Example 35

### N-(5-cyclopentyl-4-fluoro-1H-pyrazol-3-yl)-2-(3-methylisoxazol-5-yl)acetamide

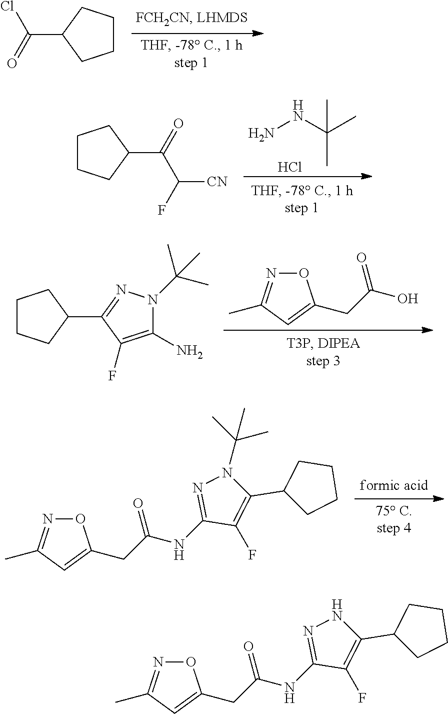

### 3-cyclopentyl-2-fluoro-3-oxopropanenitrile

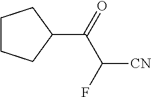 [SMILES: N#CC(F)C(=O)C1CCCC1]

Step 1. To a solution of cyclopentanecarbonyl chloride (398 mg, 365 μL, 1 Eq, 3.00 mmol) and monofluoroacetonitrile (177 mg, 167 μL, 1 Eq, 3.00 mmol) in THF (9 mL) was added LHMDS (1.00 g, 6.00 mL, 1 molar, 2 Eq, 6.00 mmol) at −78° C. The reaction was slowly warm to room temperature over 2 hr. The reaction was quenched with water, diluted with EtOAc (100 mL). The organic layer was washed with 10% citric (50 mL) acid followed by brine (50 mL). The crude was directly used in the following step without any further purification.

### 1-(tert-butyl)-3-cyclopentyl-4-fluoro-1H-pyrazol-5-amine

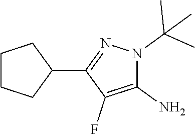 [SMILES: CC(C)(C)n1nc(C2CCCC2)c(F)c1N]

Step 2. To a solution of tert-butylhydrazine hydrochloride (561 mg, 1.5 Eq, 4.50 mmol) in EtOH (8 mL) was added NaOH (180 mg, 1.5 Eq, 4.50 mmol). The reaction was allowed to stir at room temperature for 50 min before addition of 3-cyclopentyl-2-fluoro-3-oxopropanenitrile (466 mg, 1 Eq, 3.00 mmol). Then the reaction was allowed to stir at reflux for over 2 days. The reaction was concentrated and re-dissolved in DCM (50 mL). The resulting suspension was filtered and concentrated. The crude residue was then purified by 24 g silica gel column using a mixture of EtOAc in heptane gradient (0-40%) to afford the title compound as a brown solid (213.4 mg, 947.1 μmol, 31.6%). LC-MS (ESI⁺) m z: 153.1 (M+H)⁺.

### N-(1-(tert-butyl)-5-cyclopentyl-4-fluoro-1H-pyrazol-3-yl)-2-(3-methylisoxazol-5-yl)acetamide

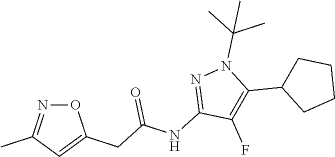 [SMILES: Cc1cc(CC(=O)Nc2nn(C(C)(C)C)c(C3CCCC3)c2F)on1]

Step 3. To a solution of 1-(tert-butyl)-5-cyclopentyl-4-fluoro-1H-pyrazol-3-amine (62.1 mg, 1 Eq, 276 μmol) in DCM (1.0 mL) was added 2-(3-methylisoxazol-5-yl)acetic acid (58.3 mg, 1.5 Eq, 413 μmol), DIPEA (107 mg, 144 μL, 3 Eq, 827 μmol) and Propylphosphonic anhydride (526 mg, 489 μL, 50% Wt, 3 Eq, 827 μmol) at ° C. The reaction was allowed to stir for 1 hour. The reaction was diluted with EtOAc (100 mL) and washed with 10% citric (50 mL) acid followed by brine (50 mL). The crude residue was then purified by 12 g silica gel column using a mixture of EtOAc in heptane gradient (0-100%) to afford the title compound (44.7 mg, 276 μmol, 49.7%). LC-MS (ESI⁺) m z: 349.40 (M+H)⁺.

### N-(5-cyclopentyl-4-fluoro-1H-pyrazol-3-yl)-2-(3-methylisoxazol-5-yl)acetamide

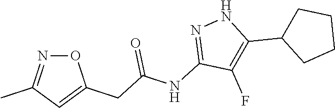 [SMILES: Cc1cc(CC(=O)Nc2n[nH]c(C3CCCC3)c2F)on1]

Step 4. A solution of N-(1-(tert-butyl)-5-cyclopentyl-4-fluoro-1H-pyrazol-3-yl)-2-(3-methylisoxazol-5-yl)acetamide (47.7 mg, 1 Eq, 137 μmol) in formic acid (1 mL) was allowed to stir at 70 degree. Concentrate and purified by prep-HPLC (20-50% ACN in water with 0.1% FA) to afford the title compound as a white solid (27.2 mg, 93.1 μmol, 68.0%). LC-MS (ESI⁺) m z: 293.33 (M+H)⁺. ¹H NMR (400 MHz, DMSO) δ 12.31 (s, 1H), 10.06 (s, 1H), 6.22 (s, 1H), 3.84 (s, 2H), 3.00 (t, J=8.4 Hz, 1H), 2.20 (s, 3H), 1.97 (br, 2H), 1.83-1.47 (m, 6H).

### Example 36

### N-(5-cyclopentylisothiazol-3-yl)-2-(3-methylisoxazol-5-yl)acetamide

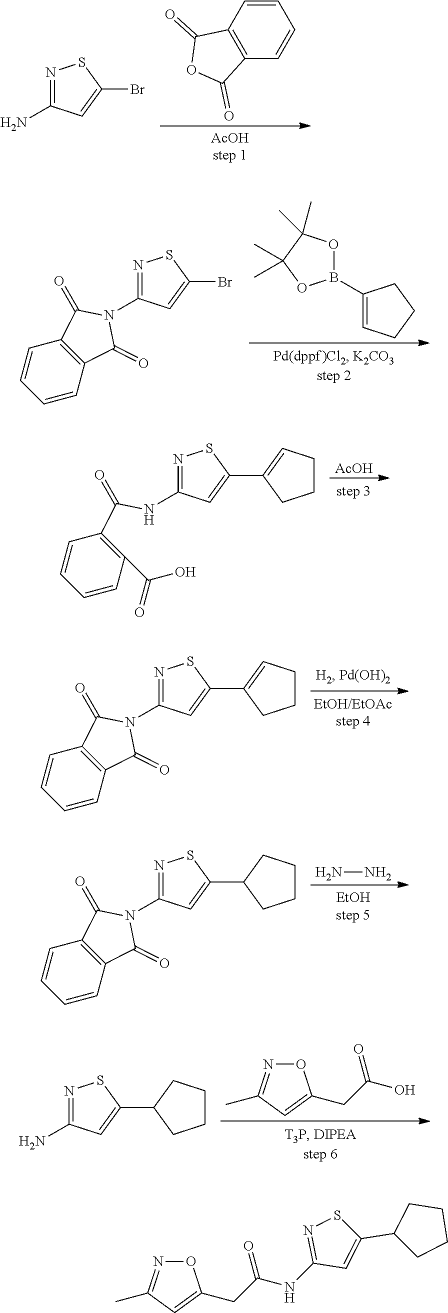

### 2-(5-(cyclopent-1-en-1-yl)isothiazol-3-yl)isoindoline-1,3-dione

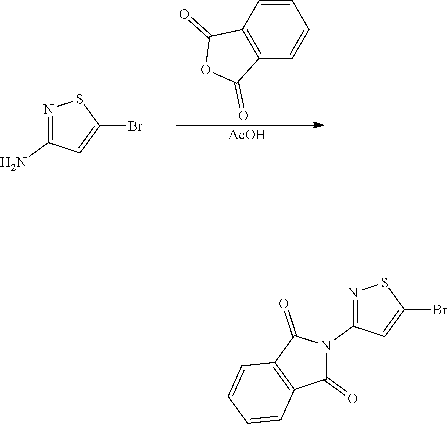 [SMILES: "AcOH" ; Nc1cc(Br)sn1 ; O=C1OC(=O)c2ccccc21 ; O=C1c2ccccc2C(=O)N1c1cc(Br)sn1]

Step 1. To a vial was added 5-bromoisothiazol-3-amine (168.2 mg, 1 Eq, 939.5 μmol), isobenzofuran-1,3-dione (139.2 mg, 1 Eq, 939.5 μmol) in Acetic Acid (1 mL). The reaction was allowed to stir at 100° C. for overnight. The crude was concentrated and used in the next step directly.

### 2-(5-(cyclopent-1-en-1-yl)isothiazol-3-yl)isoindoline-1,3-dione

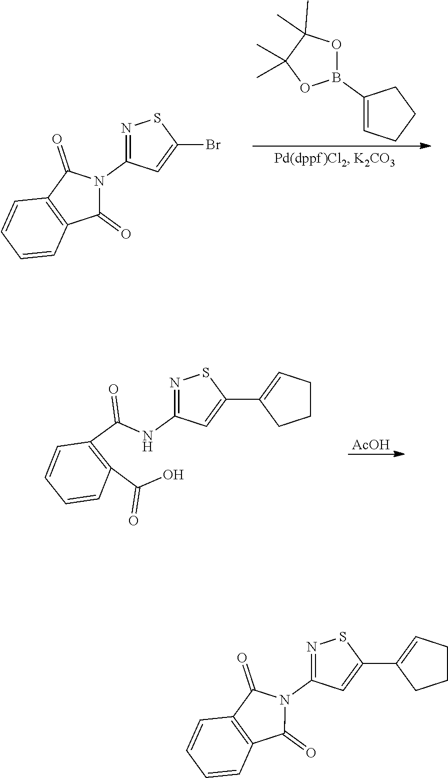

Step 2 and 3. To a vial was charged with 2-(5-bromoisothiazol-3-yl)isoindoline-1,3-dione (290 mg, 1 Eq, 0.939 mmol), Pd(dppf)Cl2 (42.8 mg, 0.07 Eq, 65.7 μmol), 2-(cyclopent-1-en-1-yl)-4,4,5,5-tetramethyl-1,3,2-dioxaborolane (273 mg, 0.28 mL, 1.5 Eq, 1.41 mmol) and potassium carbonate (324 mg, 2.5 Eq, 2.35 mmol) in mixed solvent of 1,4-Dioxane (4 mL) and Water (1 mL). The resulting reaction mixture was allowed to stir at 80° C. for 3 hours. The reaction was diluted with DCM (50 mL) and washed with 10% citric (25 mL) acid. The separated organic layer was concentrated and redissolved in AcOH (5 mL). The crude was allowed to stir at 100° c. for 3 hr. The reaction mixture was concentrated and purified by 24 g silica gel column using a mixture of EtOAc in heptane gradient (0-60%) to afford the title compound (51.2 mg, 173 μmol, 18.4%). LC-MS (ESI⁺) m z: 297.22 (M+H)⁺.

### 2-(5-cyclopentylisothiazol-3-yl)isoindoline-1,3-dione

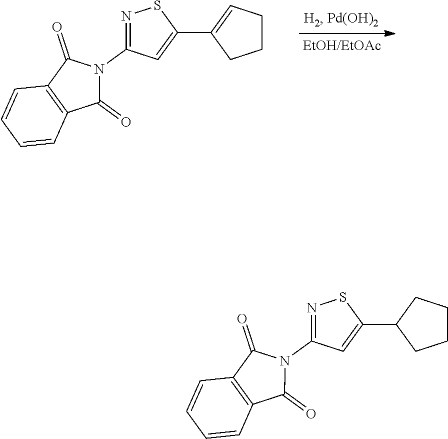 [SMILES: O=C1c2ccccc2C(=O)N1c1cc(C2=CCCC2)sn1 ; O=C1c2ccccc2C(=O)N1c1cc(C2CCCC2)sn1]

Step 4. A solution of 2-(5-(cyclopent-1-en-1-yl)isothiazol-3-yl)isoindoline-1,3-dione (57 mg, 1 Eq, 0.19 mmol) in Ethanol (5 mL) and Ethyl acetate (5 mL) was subjected to H-cube with Pd(OH)₂ cartridge at 1 ml/min flow rate, under 10 bar hydrogen pressure at 60° C. for 7 hr. The crude was concentrated to afford the title compound (14 mg, 47 μmol, 24%). LC-MS (ESI⁺) m z: 299.17 (M+H)⁺.

### 5-cyclopentylisothiazol-3-amine

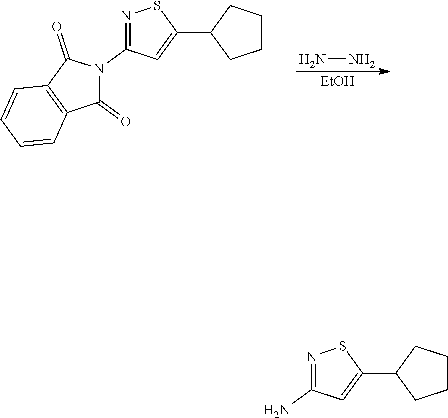

Step 5. To a solution of 2-(5-cyclopentylisothiazol-3-yl)isoindoline-1,3-dione (14 mg, 1 Eq, 47 μmol) in EtOH (0.5 mL) was added hydrazine (4.5 mg, 4.4 μL, 3 Eq, 0.14 mmol). The reaction was allowed to stir at 60° c. for 3 hr. Filtration and wash with EtOAc. The crude was used in the next step directly.

### N-(5-cyclopentylisothiazol-3-yl)-2-(3-methylisoxazol-5-yl)acetamide

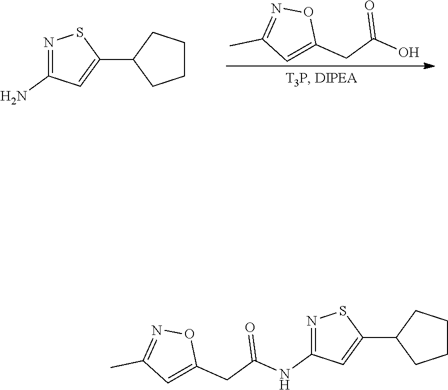

Step 6. To a vial was added 5-cyclopentylisothiazol-3-amine (8.0 mg, 1 Eq, 48 μmol)₂-(3-methylisoxazol-5-yl)acetic acid (13 mg, 2 Eq, 95 μmol), 2,4,6-tripropyl-1,3,5,2,4,6-trioxatriphosphinane 2,4,6-trioxide (0.12 g, 0.11 mL, 50% Wt, 4 Eq, 0.19 mmol) and DIPEA (25 mg, 33 μL, 4 Eq, 0.19 mmol) in DCM (1 mL) at rt. The reaction was allowed to stir for 1 hr. The reaction was diluted with EtOAc (10 mL) and washed with 10% citric (5 mL) acid followed by brine (5 mL). The crude residue was then purified by 4 g silica gel column using a mixture of EtOAc in heptane gradient (0-100%) to afford the title compound (4.0 mg, 0.01 μmol, 30%).

LC-MS (ESI⁺) m z: 292.27 (M+H)⁺.

### Example 37

### (1R,3S)-3-(3-(pyrimidin-2-ylamino)-1H-pyrazol-5-yl)cyclopentyl isopropylcarbamate

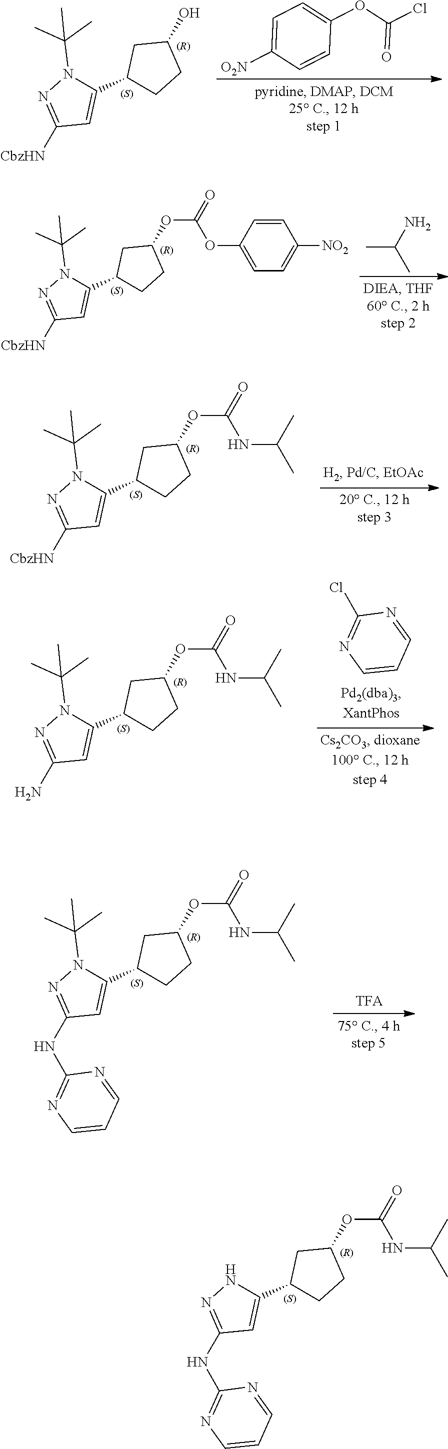

### benzyl (1-(tert-butyl)-5-((1S,3R)-3-(((4-nitrophenoxy)carbonyl)oxy)cyclopentyl)-1H-pyrazol-3-yl)carbamate

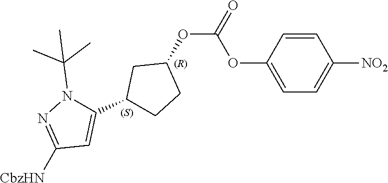

Step 1. The mixture of benzyl N-{1-tert-butyl-5-[(1R,3S)-3-hydroxycyclopentyl]-1H-pyrazol-3-yl}carbamate (500.0 mg, 1.39 mmol), pyridine (335.0 μL, 4.17 mmol), 4-nitrophenyl carbonochloridate (560.0 mg, 2.78 mmol) and DMAP (16.9 mg, 139.0 μmol) in DCM (20.0 mL) was stirred at 25° C. for 12 hours. The mixture was washed with aq·citric acid (20 mL, 10%), brine (20 mL), dried over anhydrous Na₂SO₄, filtered and concentrated to afford the crude product of (1S,3R)-3-(3-{[(benzyloxy)carbonyl]amino}-1-tert-butyl-1H-pyrazol-5-yl)cyclopentyl 4-nitrophenyl carbonate (400.0 mg, 55.0% yield) as a yellow oil which was used for next step directly. LC-MS (ESI⁺) m z: 523.4 (M+H)⁺.

### (1R,3S)-3-(3-(((benzyloxy)carbonyl)amino)-1-(tert-butyl)-1H-pyrazol-5-yl)cyclopentyl isopropylcarbamate

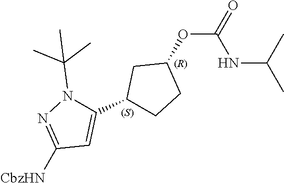

Step 2. The mixture of (1S,3R)-3-(3-{[(benzyloxy)carbonyl]amino}-1-tert-butyl-1H-pyrazol-5-yl)cyclopentyl 4-nitrophenyl carbonate (400.0 mg, 765.0 μmol), propan-2-amine (135.0 mg, 2.29 mmol) and DIEA (295.0 mg, 2.29 mmol) in THE (5.0 mL) was stirred at 60° C. for 2 hours. The mixture was concentrated to give a residue which was purified by silica gel chromatography (ethyl acetate in petroleum ether=0% to 30%) to afford the product of benzyl N-{1-tert-butyl-5-[(1R,3S)-3-{[(propan-2-yl)carbamoyl]oxy}cyclopentyl]-1H-pyrazol-3-yl}carbamate (260.0 mg, 76.9% yield) as a yellow oil. LC-MS (ESI⁺) m z: 443.6 (M+H)⁺.

### (1R,3S)-3-(3-amino-1-(tert-butyl)-1H-pyrazol-5-yl)cyclopentyl isopropylcarbamate

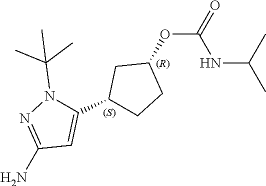

Step 3. The mixture of benzyl N-{1-tert-butyl-5-[(1R,3S)-3-{[(propan-2-yl)carbamoyl]oxy}cyclopentyl]-1H-pyrazol-3-yl}carbamate (260.0 mg, 587.0 μmol) and Pd/C (50.0 mg, 10% wet) in EtOAc (4.0 mL) was stirred at 20° C. for 12 hours under H₂ (15 Psi). The mixture was filtered and the filtrate was concentrated to afford the product of (1S,3R)-3-(3-amino-1-tert-butyl-1H-pyrazol-5-yl)cyclopentyl-N-(propan-2-yl)carbamate (150.0 mg, 82.8% yield, crude) as a yellow oil, which was used for next step directly. LC-MS (ESI⁺) m z: 309.3 (M+H)⁺.

### (1R,3S)-3-(1-(tert-butyl)-3-(pyrimidin-2-ylamino)-1H-pyrazol-5-yl)cyclopentyl isopropylcarbamate

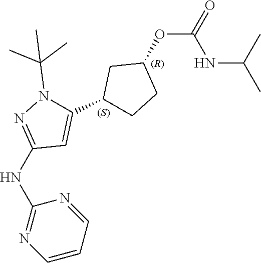

Step 4 The mixture of (1S,3R)-3-(3-amino-1-(tert-butyl)-1H-pyrazol-5-yl)cyclopentyl-isopropylcarbamate (110.0 mg, 356.6 μmol), 2-chloropyrimidine (49.0 mg, 428.0 μmol), XantPhos (41.3 mg, 71.3 μmol), Pd₂(dba)₃ (32.7 mg, 35.7 μmol) and Cs₂CO₃ (232.4 mg, 713.3 μmol) in dioxane (4.0 mL) was stirred at 100° C. for 12 hours under N₂ protection. The mixture was diluted with ethyl acetate (20 mL) and H₂O (20 mL). The aqueous layer was separated and extracted with ethyl acetate (20 mL×2). The combined organic layers were washed with brine (20 mL), dried over anhydrous Na₂SO₄, filtered and concentrated to give a residue which was purified by silica gel chromatography (ethyl acetate in petroleum ether=0% to 60%) to afford the product of (1S,3R)-3-(1-(tert-butyl)-3-(pyrimidin-2-ylamino)-1H-pyrazol-5-yl)cyclopentyl-isopropylcarbamate (80.0 mg, 58.0% yield) as a yellow solid. LC-MS (ESI⁺) m z: 387.3 (M+H)⁺.

### (1R,3S)-3-(3-(pyrimidin-2-ylamino)-1H-pyrazol-5-yl)cyclopentyl isopropylcarbamate

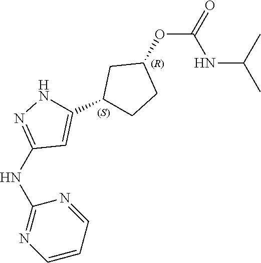

Step 5. The mixture of (1R,3S)-3-(1-(tert-butyl)-3-(pyrimidin-2-ylamino)-1H-pyrazol-5-yl)cyclopentyl-isopropylcarbamate (60.0 mg, 155.0 μmol) in TFA (2.0 mL) was stirred at 75° C. for 4 hours. The mixture was concentrated to give a residue which was purified by prep-HPLC (NH₃ H₂O) to afford the product of (1R,3S)-3-(3-(pyrimidin-2-ylamino)-1H-pyrazol-5-yl)cyclopentyl-isopropylcarbamate (27.2 mg, 53.0% yield) as a white solid. LC-MS (ESI⁺) m z: 331.1 (M+H)⁺. ¹H NMR (400 MHz, DMSO-d₆) δ ppm 11.92 (s, 1H), 9.48 (s, 1H), 8.41 (d, J=3.2 Hz, 2H), 6.95 (d, J=6.8 Hz, 1H), 6.76 (s, 1H), 6.41 (s, 1H), 5.00 (s, 1H), 3.54-3.62 (m, 1H), 3.05 (s, 1H), 2.40-2.49 (m, 1H), 2.01 (s, 1H), 1.83-1.93 (m, 1H), 1.73 (d, J=7.6 Hz, 2H), 1.63 (s, 1H), 1.03 (d, J=6.4 Hz, 6H).

Additional compounds prepared according to the methods of Example 37 are depicted in Table 13 below.

TABLE 13
Additional Exemplary Compounds

| Compound | Structure | MS [M + 1] |
|---|---|---|
| 4-((5-cyclopentyl-1H- pyrazol-3-yl)amino)-6- methyl-1H-pyrrolo[3,4- c]pyridine-1,3(2H)-dione | 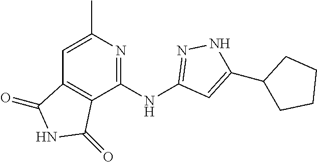 [SMILES: Cc1cc2c(c(Nc3cc(C4CCCC4)[nH]n3)n1)C(=O)NC2=O] | 312.2 |
| N-(5-cyclopentyl-1H- pyrazol-3-yl)imidazo[1,2- b]pyridazin-7-amine | 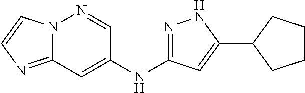 [SMILES: c1cn2ncc(Nc3cc(C4CCCC4)[nH]n3)cc2n1] | 269.2 |
| N-(5-cyclopentyl-1H- pyrazol-3-yl)-5,6,7,8- tetrahydro-5,8- methanoquinazolin-2-amine | 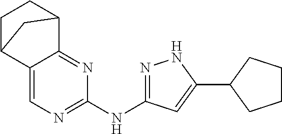 [SMILES: c1c(Nc2ncc3c(n2)C2CCC3C2)n[nH]c1C1CCCC1] | 296 |
| N-(5-cyclopentyl-1H- pyrazol-3-yl)-6,8- dimethylpyrrolo[1,2- a]pyrazin-1-amine | 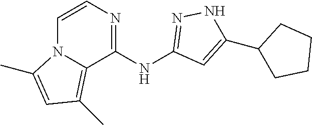 [SMILES: Cc1cc(C)n2ccnc(Nc3cc(C4CCCC4)[nH]n3)c12] | 296 |
| 2-((5-cyclopentyl-1H- pyrazol-3-yl)amino)-7,7- dimethyl-6,7-dihydro-5H- pyrrolo[3,4-b]pyridin-5-one | 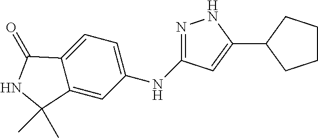 [SMILES: CC1(C)NC(=O)c2ccc(Nc3cc(C4CCCC4)[nH]n3)cc21] | 312 |
| N-(5-cyclopentyl-1H- pyrazol-3-yl)-5-methyl- [1,2,4]triazolo[4,3- c]pyrimidin-7-amine | 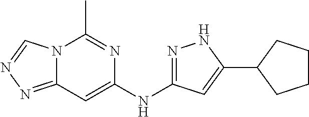 [SMILES: Cc1nc(Nc2cc(C3CCCC3)[nH]n2)cc2nncn12] | 284 |
| 2-(4-((5-cyclopentyl-1H- pyrazol-3-yl)amino)-1H- pyrrolo[2,3-b]pyridin-3- yl)acetonitrile | 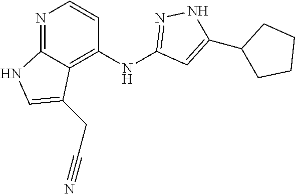 [SMILES: N#CCc1c[nH]c2nccc(Nc3cc(C4CCCC4)[nH]n3)c12] | 307.4 |
| 3-chloro-N-(5-cyclopentyl- 1H-pyrazol-3-yl)-5- methylpyridin-4-amine | 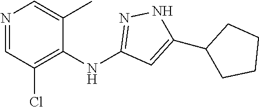 [SMILES: Cc1cncc(Cl)c1Nc1cc(C2CCCC2)[nH]n1] | 277.2 |
| methyl 5-((5-cyclopentyl- 1H-pyrazol-3- yl)amino)pyridazine-3- carboxylate | 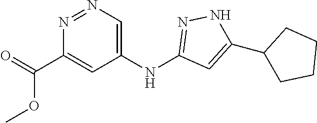 [SMILES: COC(=O)c1cc(Nc2cc(C3CCCC3)[nH]n2)cnn1] | 288 |
| 5-chloro-N-(5-cyclopentyl- 1H-pyrazol-3-yl)quinoxalin- 2-amine | 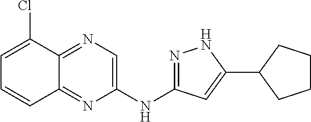 [SMILES: Clc1cccc2nc(Nc3cc(C4CCCC4)[nH]n3)cnc12] | 314 |
| N-(5-cyclopentyl-1H- pyrazol-3-yl)-2,5- dimethylpyrazolo[1,5- a]pyrimidin-7-amine | 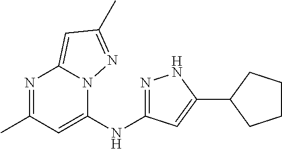 [SMILES: Cc1cc(Nc2cc(C3CCCC3)[nH]n2)n2nc(C)cc2n1] | 297.4 |
| methyl 4-((5-cyclopentyl- 1H-pyrazol-3-yl)amino)-6- methylnicotinate | 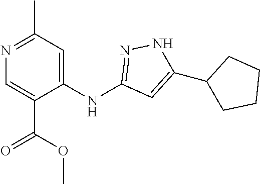 [SMILES: COC(=O)c1cnc(C)cc1Nc1cc(C2CCCC2)[nH]n1] | 301.4 |
| N-(5-cyclopentyl-1H- pyrazol-3-yl)-3-methoxy- 1,7-naphthyridin-8-amine | 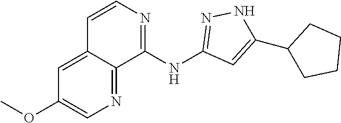 [SMILES: COc1cnc2c(Nc3cc(C4CCCC4)[nH]n3)nccc2c1] | 310.4 |
| N-(5-cyclopentyl-1H- pyrazol-3-yl)-5- methylthieno[2,3- d]pyrimidin-2-amine | 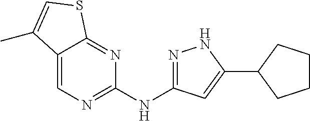 [SMILES: Cc1csc2nc(Nc3cc(C4CCCC4)[nH]n3)ncc12] | 300.2 |
| N-(5-cyclopentyl-1H- pyrazol-3-yl)-4,6- dimethylpyrimidin-2-amine | 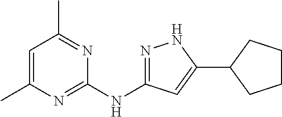 [SMILES: Cc1cc(C)nc(Nc2cc(C3CCCC3)[nH]n2)n1] | 258.4 |
| N-(5-cyclopentyl-1H- pyrazol-3-yl)-5- propylpyridazin-3-amine | 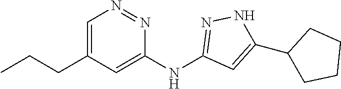 [SMILES: CCCc1cnnc(Nc2cc(C3CCCC3)[nH]n2)c1] | 272.4 |
| 5-butoxy-N-(5-cyclopentyl- 1H-pyrazol-3-yl)pyridin-2- amine | 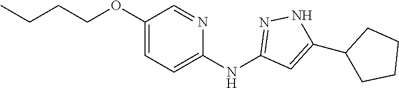 [SMILES: CCCCOc1ccc(Nc2cc(C3CCCC3)[nH]n2)nc1] | 301.2 |
| N-(5-cyclopentyl-1H- pyrazol-3-yl)-5,7-dimethyl- 7,8-dihydro-5H-pyrano[4,3- d]pyrimidin-2-amine | 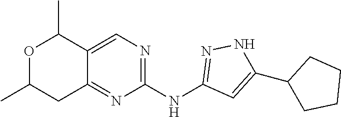 [SMILES: CC1Cc2nc(Nc3cc(C4CCCC4)[nH]n3)ncc2C(C)O1] | 314.2 |
| N-(5-cyclopentyl-1H- pyrazol-3-yl)-4- methoxythieno[2,3- b]pyridin-6-amine | 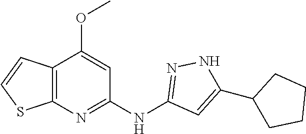 [SMILES: COc1cc(Nc2cc(C3CCCC3)[nH]n2)nc2sccc12] | 315.2 |
| N-(5-cyclopentyl-1H- pyrazol-3-yl)-4-(2- methoxyethoxy)pyrimidin- 2-amine | 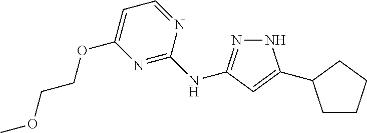 [SMILES: COCCOc1ccnc(Nc2cc(C3CCCC3)[nH]n2)n1] | 304.4 |
| N-(5-cyclopentyl-1H- pyrazol-3-yl)-5-fluoro-2- methylquinolin-4-amine | 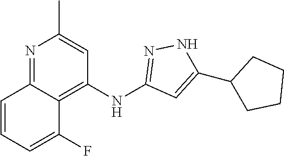 [SMILES: Cc1cc(Nc2cc(C3CCCC3)[nH]n2)c2c(F)cccc2n1] | 311.2 |
| 1-((5-cyclopentyl-1H- pyrazol-3- yl)amino)isoquinolin-7-ol | 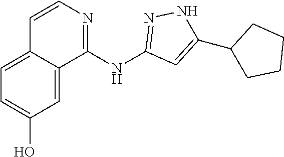 [SMILES: Oc1ccc2ccnc(Nc3cc(C4CCCC4)[nH]n3)c2c1] | 295.2 |
| N-(5-cyclopentyl-1H- pyrazol-3-yl)pyrimidin-2- amine | 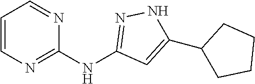 [SMILES: c1cnc(Nc2cc(C3CCCC3)[nH]n2)nc1] | 230.2 |
| N-(5-cyclopentyl-1H- pyrazol-3-yl)-5,6,7,8- tetrahydrophthalazin-1- amine | 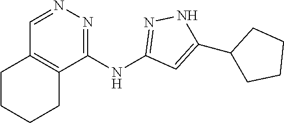 [SMILES: c1c(Nc2nncc3c2CCCC3)n[nH]c1C1CCCC1] | 284.2 |
| 4-((5-cyclopentyl-1H- pyrazol-3- yl)amino)quinoline-3- carbonitrile | 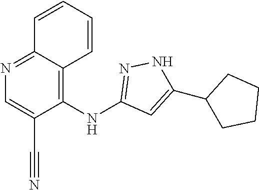 [SMILES: N#Cc1cnc2ccccc2c1Nc1cc(C2CCCC2)[nH]n1] | 418.5 |
| N-(5-cyclopentyl-1H- pyrazol-3-yl)-5-isopropyl- [1,2,4]triazolo[1,5- alpyrimidin-7-amine | 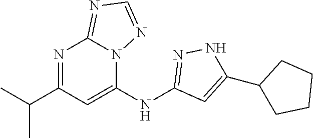 [SMILES: CC(C)c1cc(Nc2cc(C3CCCC3)[nH]n2)n2ncnc2n1] | 312.2 |
| N-(5-cyclopentyl-1H- pyrazol-3-yl)-5-fluoro-6- methylquinolin-4-amine | 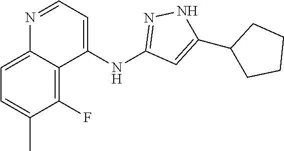 [SMILES: Cc1ccc2nccc(Nc3cc(C4CCCC4)[nH]n3)c2c1F] | 311.2 |
| (5-((5-cyclopentyl-1H- pyrazol-3- yl)amino)furo[3,2-b]pyridin- 2-yl)methanol | 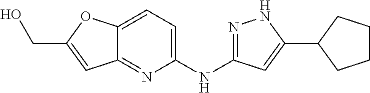 [SMILES: OCc1cc2nc(Nc3cc(C4CCCC4)[nH]n3)ccc2o1] | 299.34 |
| 7-((5-cyclopentyl-1H- pyrazol-3- yl)amino)thieno[2,3- c]pyridine-4-carbonitrile | 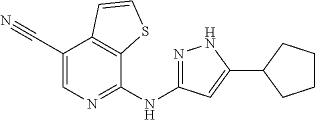 [SMILES: N#Cc1cnc(Nc2cc(C3CCCC3)[nH]n2)c2sccc12] | 310 |
| N-(5-cyclopentyl-1H- pyrazol-3-yl)-7- methylthieno[3,2-b]pyridin- 5-amine | 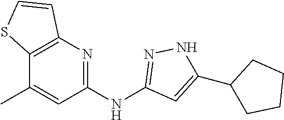 [SMILES: Cc1cc(Nc2cc(C3CCCC3)[nH]n2)nc2ccsc12] | 199.4 |
| N-(5-cyclopentyl-1H- pyrazol-3-yl)-6- methylpyrazolo[1,5- a]pyrazin-4-amine | 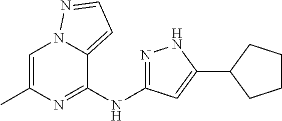 [SMILES: Cc1cn2nccc2c(Nc2cc(C3CCCC3)[nH]n2)n1] | 283.2 |
| N-(5-cyclopentyl-1H- pyrazol-3-yl)-4-ethoxy-5- methylpyrimidin-2-amine | 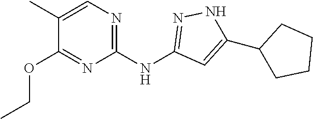 [SMILES: CCOc1nc(Nc2cc(C3CCCC3)[nH]n2)ncc1C] | 288.6 |
| N-(5-cyclopentyl-1H- pyrazol-3-yl)-1-methyl-6,7- dihydro-5H- cyclopenta[c]pyridin-3- amine | 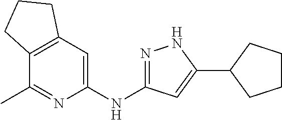 [SMILES: Cc1nc(Nc2cc(C3CCCC3)[nH]n2)cc2c1CCC2] | 283.2 |
| N-(5-cyclopentyl-1H- pyrazol-3-yl)-4- methoxyquinazolin-2-amine | 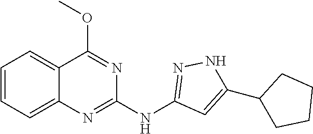 [SMILES: COc1nc(Nc2cc(C3CCCC3)[nH]n2)nc2ccccc12] | 310 |
| 6-((5-cyclopentyl-1H- pyrazol-3-yl)amino)-1H- pyrido[2,3-b][1,4]oxazin- 2(3H)-one | 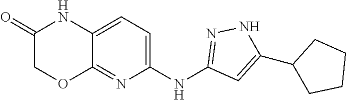 [SMILES: O=C1COc2nc(Nc3cc(C4CCCC4)[nH]n3)ccc2N1] | 300.2 |
| N-(5-cyclopentyl-1H- pyrazol-3-yl)-3-methyl-2H- pyrazolo[3,4-b]pyridin-4- amine | 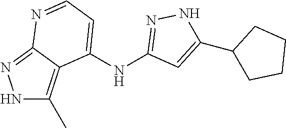 [SMILES: Cc1[nH]nc2nccc(Nc3cc(C4CCCC4)[nH]n3)c12] | 283.2 |
| N-(5-cyclopentyl-1H- pyrazol-3-yl)-5- methylquinazolin-4-amine | 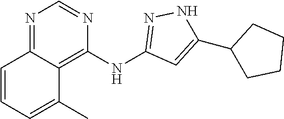 [SMILES: Cc1cccc2ncnc(Nc3cc(C4CCCC4)[nH]n3)c12] | 294.5 |
| N-(5-cyclopentyl-1H- pyrazol-3-yl)-5-methoxy-2- methylpyrimidin-4-amine | 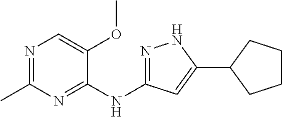 [SMILES: COc1cnc(C)nc1Nc1cc(C2CCCC2)[nH]n1] | 274.2 |
| N-(5-cyclopentyl-1H- pyrazol-3-yl)-7,7-dimethyl- 6,7-dihydro-5H- cyclopenta[c]pyridazin-3- amine | 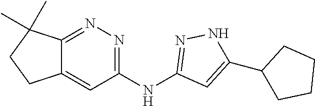 [SMILES: CC1(C)CCc2cc(Nc3cc(C4CCCC4)[nH]n3)nnc21] | 298.4 |
| 4-((5-cyclopentyl-1H- pyrazol-3-yl)amino)-5H- pyrimido[4,5-b][1,4]oxazin- 6(7H)-one | 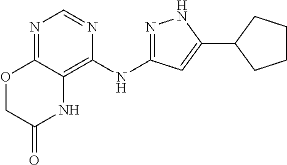 [SMILES: O=C1COc2ncnc(Nc3cc(C4CCCC4)[nH]n3)c2N1] | 301.4 |
| N-(5-cyclopentyl-1H- pyrazol-3-yl)-2,8- dimethylimidazo[1,2- b]pyridazin-6-amine | 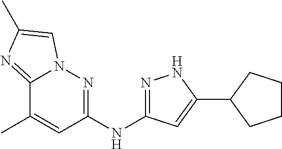 [SMILES: Cc1cn2nc(Nc3cc(C4CCCC4)[nH]n3)cc(C)c2n1] | 297.2 |
| N-(5-cyclopentyl-1H- pyrazol-3-yl)-4-methyl-6- (1H-pyrazol-1-yl)pyrimidin- 2-amine | 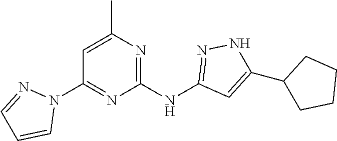 [SMILES: Cc1cc(-n2cccn2)nc(Nc2cc(C3CCCC3)[nH]n2)n1] | 300.2 |
| N-(5-cyclopentyl-1H- pyrazol-3-yl)-5-isobutyl-4,6- dimethylpyrimidin-2-amine | 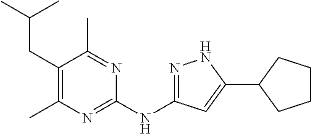 [SMILES: Cc1nc(Nc2cc(C3CCCC3)[nH]n2)nc(C)c1CC(C)C] | 314.2 |
| 4-((5-cyclopentyl-1H- pyrazol-3- yl)amino)cinnoline-3- carbonitrile | 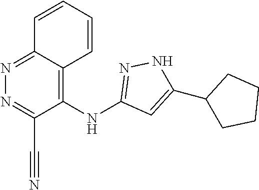 [SMILES: N#Cc1nnc2ccccc2c1Nc1cc(C2CCCC2)[nH]n1] | 305.5 |
| N-(5-cyclopentyl-1H- pyrazol-3-yl)-4- methylphthalazin-1-amine | 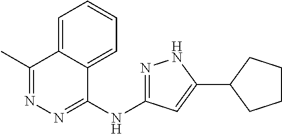 [SMILES: Cc1nnc(Nc2cc(C3CCCC3)[nH]n2)c2ccccc12] | 294.4 |
| N-(5-cyclopentyl-1H- pyrazol-3-yl)imidazo[1,2- a]pyrimidin-5-amine | 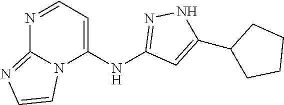 [SMILES: c1cc(Nc2cc(C3CCCC3)[nH]n2)n2ccnc2n1] | 269.2 |
| 2-((5-cyclopentyl-1H- pyrazol-3- yl)amino)thieno[3,2- d]pyrimidin-4(3H)-one | 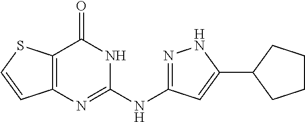 [SMILES: O=c1[nH]c(Nc2cc(C3CCCC3)[nH]n2)nc2ccsc12] | 302.2 |
| N-(5-cyclopentyl-1H- pyrazol-3-yl)-2- methylthieno[3,2- d]pyrimidin-4-amine | 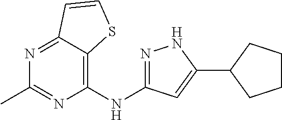 [SMILES: Cc1nc(Nc2cc(C3CCCC3)[nH]n2)c2sccc2n1] | 300.2 |
| 4-((5-cyclopentyl-1H- pyrazol-3-yl)amino)-1,1- dimethylfuro[3,4-c]pyridin- 3(1H)-one | 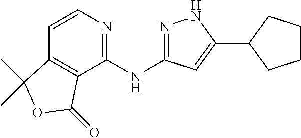 [SMILES: CC1(C)OC(=O)c2c1ccnc2Nc1cc(C2CCCC2)[nH]n1] | 313.2 |
| N-(5-cyclopentyl-1H- pyrazol-3-yl)-3- methoxyquinolin-2-amine | 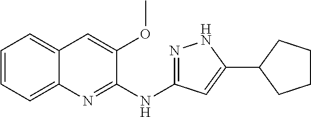 [SMILES: COc1cc2ccccc2nc1Nc1cc(C2CCCC2)[nH]n1] | 309.2 |
| 2-((6-((5-cyclopentyl-1H- pyrazol-3-yl)amino)pyrazin- 2-yl)oxy)ethan-1-ol | 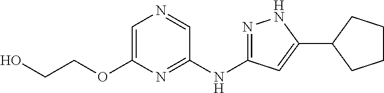 [SMILES: OCCOc1cncc(Nc2cc(C3CCCC3)[nH]n2)n1] | 290.2 |
| 4-((5-cyclopentyl-1H- pyrazol-3-yl)amino)-5,6,7,8- tetrahydroquinolin-8-ol | 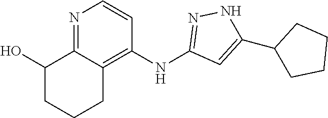 [SMILES: OC1CCCc2c(Nc3cc(C4CCCC4)[nH]n3)ccnc21] | 299 |
| N-(5-cyclopentyl-1H- pyrazol-3-yl)-4-(1H- imidazol-2-yl)pyridin-2- amine | 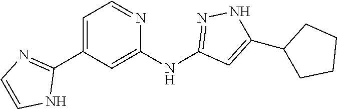 [SMILES: c1cc(-c2ncc[nH]2)cc(Nc2cc(C3CCCC3)[nH]n2)n1] | 295.3 |
| N-(5-cyclopentyl-1H- pyrazol-3-yl)-6- methylisoquinolin-3-amine | 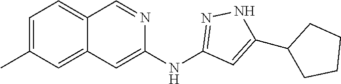 [SMILES: Cc1ccc2cnc(Nc3cc(C4CCCC4)[nH]n3)cc2c1] | 293.5 |
| N-(5-cyclopentyl-1H- pyrazol-3-yl)-3-cyclopropyl- 2H-pyrazolo[3,4-b]pyridin- 6-amine | 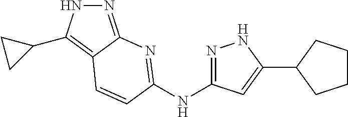 [SMILES: c1cc2c(C3CC3)[nH]nc2nc1Nc1cc(C2CCCC2)[nH]n1] | 308.3 |
| N-(5-cyclopentyl-1H- pyrazol-3-yl)-3,5- dimethoxypyridazin-4- amine | 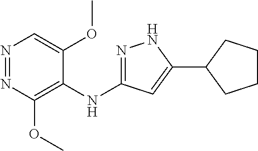 [SMILES: COc1cnnc(OC)c1Nc1cc(C2CCCC2)[nH]n1] | 289.3 |
| 4-((5-cyclopentyl-1H- pyrazol-3- yl)amino)pyridine-3- sulfonamide | 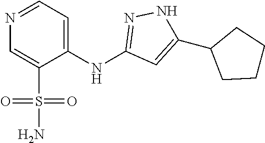 [SMILES: NS(=O)(=O)c1cnccc1Nc1cc(C2CCCC2)[nH]n1] | 308.4 |
| 6-((5-cyclopentyl-1H- pyrazol-3-yl)amino)-1,5- naphthyridin-2(1H)-one | 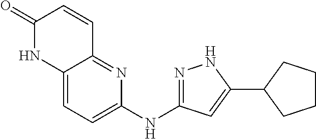 [SMILES: O=c1ccc2nc(Nc3cc(C4CCCC4)[nH]n3)ccc2[nH]1] | 296.3 |
| 7-chloro-N-(5-cyclopentyl- 1H-pyrazol-3-yl)- [1,2,4]triazolo[4,3-a]pyridin- 3-amine | 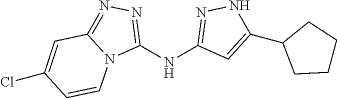 [SMILES: Clc1ccn2c(Nc3cc(C4CCCC4)[nH]n3)nnc2c1] | 303 |
| N-(5-cyclopentyl-1H- pyrazol-3-yl)-3-isopropyl- [1,2,4]triazolo[4,3- a]pyrazin-8-amine | 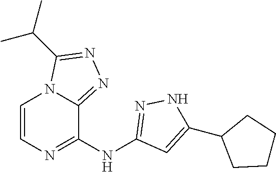 [SMILES: CC(C)c1nnc2c(Nc3cc(C4CCCC4)[nH]n3)nccn12] | 312.5 |
| N-(5-cyclopentyl-1H- pyrazol-3-yl)-5- methylimidazo[5,1- f][1,2,4]triazin-4-amine | 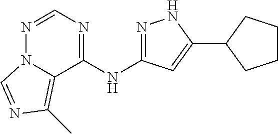 [SMILES: Cc1ncn2ncnc(Nc3cc(C4CCCC4)[nH]n3)c12] | 284.2 |
| N-(5-cyclopentyl-1H- pyrazol-3-yl)-6-(furan-2-yl)- 2-methylpyrimidin-4-amine | 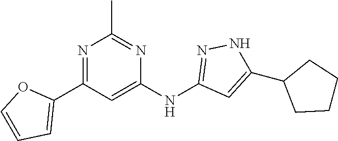 [SMILES: Cc1nc(Nc2cc(C3CCCC3)[nH]n2)cc(-c2ccco2)n1] | 310.3 |
| 3-((5-cyclopentyl-1H- pyrazol-3- yl)amino)quinoxalin-2(1H)- one | 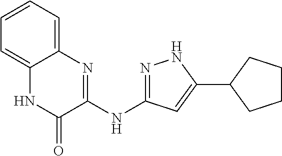 [SMILES: O=c1[nH]c2ccccc2nc1Nc1cc(C2CCCC2)[nH]n1] | 296.3 |
| N-(5-cyclopentyl-1H- pyrazol-3-yl)-6-methyl-4- (trifluoromethyl)pyridazin- 3-amine | 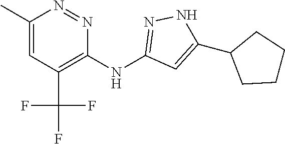 [SMILES: Cc1cc(C(F)(F)F)c(Nc2cc(C3CCCC3)[nH]n2)nn1] | 312 |
| 3-((5-cyclopentyl-1H- pyrazol-3-yl)amino)-5- methylpyridazine-4- carboxylic acid | 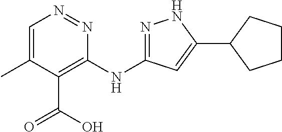 [SMILES: Cc1cnnc(Nc2cc(C3CCCC3)[nH]n2)c1C(=O)O] | 288.4 |
| N-(5-cyclopentyl-1H- pyrazol-3-yl)-1- (methylsulfonyl)piperidin-4- amine | 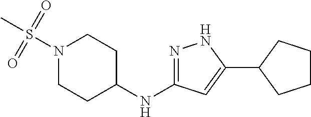 [SMILES: CS(=O)(=O)N1CCC(Nc2cc(C3CCCC3)[nH]n2)CC1] | 313.319 |
| N-(5-cyclopentyl-1H- pyrazol-3-yl)-3- methylisoquinolin-1-amine | 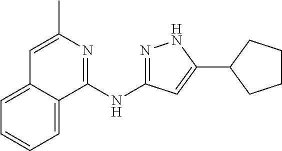 [SMILES: Cc1cc2ccccc2c(Nc2cc(C3CCCC3)[nH]n2)n1] | 293.5 |
| N-(5-cyclopentyl-1H- pyrazol-3-yl)-6-methyl-5H- pyrrolo[3,2-d]pyrimidin-4- amine | 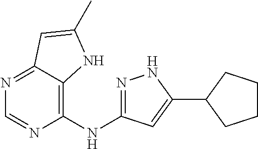 [SMILES: Cc1cc2ncnc(Nc3cc(C4CCCC4)[nH]n3)c2[nH]1] | 283.4 |
| N-(5-cyclopentyl-1H- pyrazol-3-yl)-4- fluoroisoquinolin-1-amine | 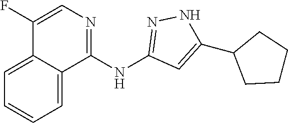 [SMILES: Fc1cnc(Nc2cc(C3CCCC3)[nH]n2)c2ccccc12] | 297.2 |
| N-(5-cyclopentyl-1H- pyrazol-3-yl)pyrrolo[2,1- f][1,2,4]triazin-4-amine | 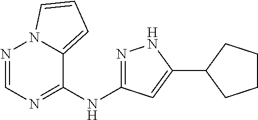 [SMILES: c1cc2c(Nc3cc(C4CCCC4)[nH]n3)ncnn2c1] | 299.2 |
| N-(5-cyclopentyl-1H- pyrazol-3-yl)-3,5- dimethylpyrazin-2-amine | 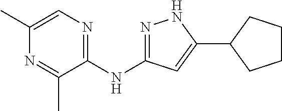 [SMILES: Cc1cnc(Nc2cc(C3CCCC3)[nH]n2)c(C)n1] | 309.2 |
| N-(5-cyclopentyl-1H- pyrazol-3-yl)-3-fluoro-4- methylquinolin-2-amine | 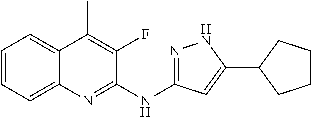 [SMILES: Cc1c(F)c(Nc2cc(C3CCCC3)[nH]n2)nc2ccccc12] | 311.2 |
| N-(5-cyclopentyl-1H- pyrazol-3-yl)-2-isopropyl- 7H-pyrrolo[2,3-d]pyrimidin- 4-amine | 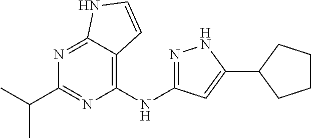 [SMILES: CC(C)c1nc(Nc2cc(C3CCCC3)[nH]n2)c2cc[nH]c2n1] | 311.4 |
| N-(5-cyclopentyl-1H- pyrazol-3-yl)-6- methoxypyrido[2,3- d]pyrimidin-4-amine | 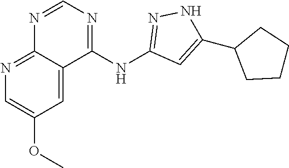 [SMILES: COc1cnc2ncnc(Nc3cc(C4CCCC4)[nH]n3)c2c1] | 285.2 |
| N-(5-cyclopentyl-1H- pyrazol-3-yl)-7,8-dimethyl- [1,2,4]triazolo[4,3- b]pyridazin-6-amine | 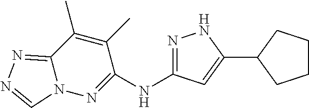 [SMILES: Cc1c(Nc2cc(C3CCCC3)[nH]n2)nn2cnnc2c1C] | 296.2 |
| N-(5-cyclopentyl-1H- pyrazol-3-yl)-7,8-dihydro- 5H-pyrano[4,3-b]pyridin-2- amine | 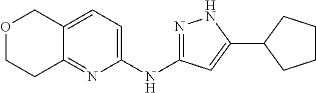 [SMILES: c1cc2c(nc1Nc1cc(C3CCCC3)[nH]n1)CCOC2] | 258.4 |
| N-(5-cyclopentyl-1H- pyrazol-3-yl)-8- methoxyquinolin-2-amine | 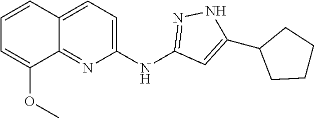 [SMILES: COc1cccc2ccc(Nc3cc(C4CCCC4)[nH]n3)nc12] | 309.2 |
| N-(5-cyclopentyl-1H- pyrazol-3-yl)-8,9-dihydro- 7H-cyclopenta[3,4] pyrazolo[1,5- a]pyrazin-1-amine | 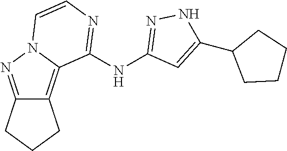 [SMILES: c1cn2nc3c(c2c(Nc2cc(C4CCCC4)[nH]n2)n1)CCC3] | 308.2 |
| N-(5-cyclopentyl-1H- pyrazol-3-yl)-2- cyclopropylpyrazolo[1,5- a]pyrazin-4-amine | 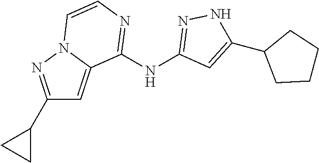 [SMILES: c1cn2nc(C3CC3)cc2c(Nc2cc(C3CCCC3)[nH]n2)n1] | 269.4 |
| N-(5-cyclopentyl-1H- pyrazol-3-yl)-4-phenyl- 1,3,5-triazin-2-amine | 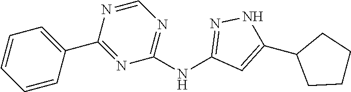 [SMILES: c1ccc(-c2ncnc(Nc3cc(C4CCCC4)[nH]n3)n2)cc1] | 307.2 |
| 3-((5-cyclopentyl-1H- pyrazol-3-yl)amino)-1- methyl-6,7-dihydro-5H- cyclopenta[c]pyridine-4- carbonitrile | 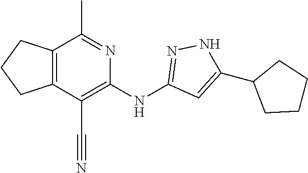 [SMILES: Cc1nc(Nc2cc(C3CCCC3)[nH]n2)c(C#N)c2c1CCC2] | 311 |
| 4-((5-cyclopentyl-1H- pyrazol-3-yl)amino)-1,5- naphthyridin-2(1H)-one | 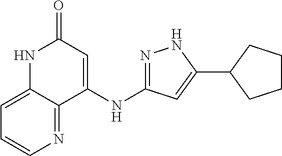 [SMILES: O=c1cc(Nc2cc(C3CCCC3)[nH]n2)c2ncccc2[nH]1] | 309.2 |
| 2-((5-cyclopentyl-1H- pyrazol-3-yl)amino)-5,6- dimethylnicotinic acid | 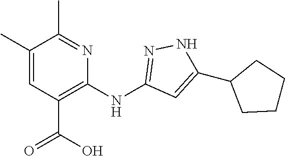 [SMILES: Cc1cc(C(=O)O)c(Nc2cc(C3CCCC3)[nH]n2)nc1C] | 301.2 |
| N-(5-cyclopentyl-1H- pyrazol-3-yl)-6-ethyl-5- methyl-[1,2,4]triazolo[1,5- alpyrimidin-7-amine | 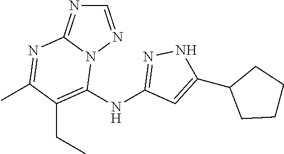 [SMILES: CCc1c(C)nc2ncnn2c1Nc1cc(C2CCCC2)[nH]n1] | 312.2 |
| 6-((5-cyclopentyl-1H- pyrazol-3- yl)amino)pyrimidin-4-ol | 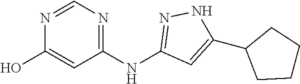 [SMILES: Oc1cc(Nc2cc(C3CCCC3)[nH]n2)ncn1] | 246.2 |
| N-(5-cyclopentyl-1H- pyrazol-3-yl)pyrazolo[1,5- alpyrimidin-5-amine | 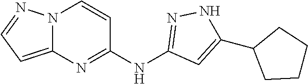 [SMILES: c1cc2nc(Nc3cc(C4CCCC4)[nH]n3)ccn2n1] | 269.2 |
| N-(5-cyclopentyl-1H- pyrazol-3-yl)-7- methoxyphthalazin-1- aminez | 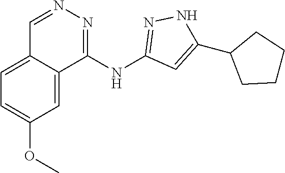 [SMILES: COc1ccc2cnnc(Nc3cc(C4CCCC4)[nH]n3)c2c1] | 310.2 |

### Example 38

### rel-(1R,3S)-3-(5-(2-(methyl(7H-purin-6-yl)amino)acetamido)-1H-pyrazol-3-yl)cyclopentyl isopropylcarbamate

### rel-(1S,3R)-3-(5-(2-(methyl(7H-purin-6-yl)amino)acetamido)-1H-pyrazol-3-yl)cyclopentyl isopropylcarbamate

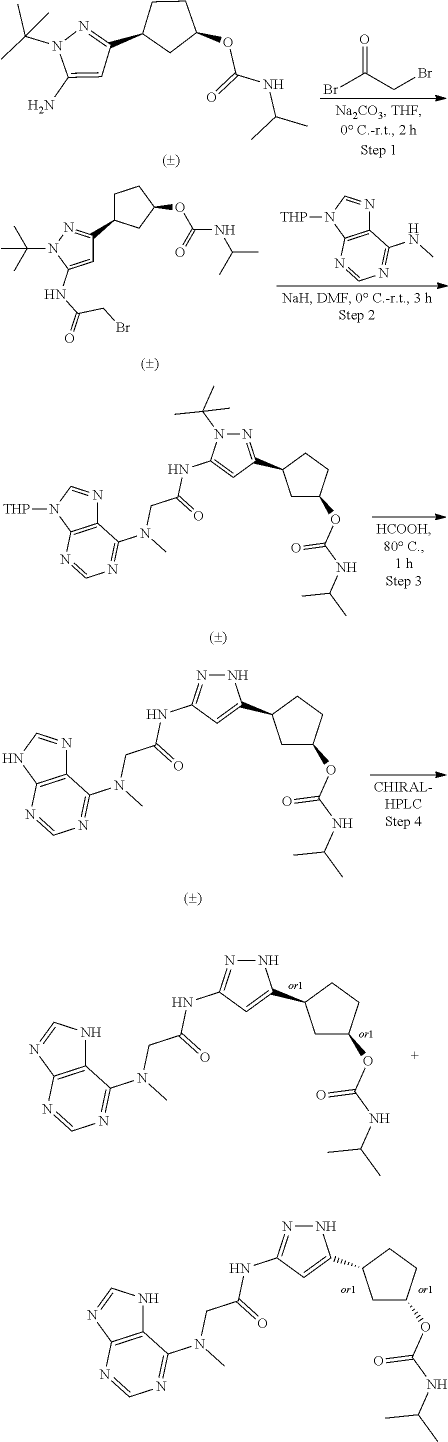

### rel-(1R,3S)-3-(5-(2-bromoacetamido)-1-(tert-butyl)-1H-pyrazol-3-yl)cyclopentyl isopropylcarbamate

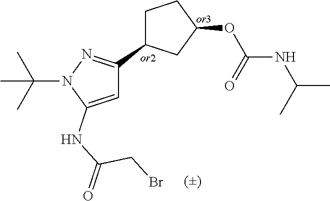 [SMILES: CC(C)NC(=O)O[C@@H]1CC[C@H](c2cc(NC(=O)CBr)n(C(C)(C)C)n2)C1]

Step 1. To a solution of rel 3-(5-amino-1-(tert-butyl)-1H-pyrazol-3-yl) cyclopentyl isopropylcarbamate (110 mg, 1 Eq, 357 μmol) in THE (3 mL), 2-bromoacetyl bromide (144 mg, 2 Eq, 713 μmol) and Na₂CO₃ (75.6 mg, 2 Eq, 713 μmol) were added. The mixture was stirred at room temperature for 2 h. LCMS was OK. The mixture was evaporated, diluted with ethyl acetate (3×30 mL) and washed with water, dried over Na₂SO₄ and evaporated in vacuo. The crude residue was purified by flash (Mobile Phase A: Water, Mobile Phase B: ACN; Flow rate: 60 mL/min; Gradient: 30% B to 45% B in 7 min); After solvent evaporation afforded the title compound rel −3-(5-(2-bromoacetamido)-1-(tert-butyl)-1H-pyrazol-3-yl) cyclopentyl isopropylcarbamate (103 mg, 240 μmol, 67.3%) as light yellow oil. m/z (ES⁺) [M+H]⁺=429.00; HPLC tR=1.131 min.

### rel-(1R,3S)-3-(1-(tert-butyl)-5-(2-(methyl(9-(tetrahydro-2H-pyran-2-yl)-9H-purin-6-yl)amino)acetamido)-1H-pyrazol-3-yl)cyclopentyl isopropylcarbamate

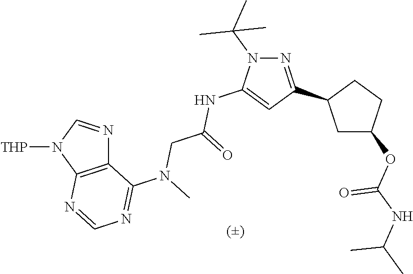

Step 2. To an ice cold solution of rel −3-(5-(2-bromoacetamido)-1-(tert-butyl)-1H-pyrazol-3-yl) cyclopentyl isopropylcarbamate (103 mg, 1 Eq, 240 μmol) in DMF (2 mL), NaH (28.8 mg, 60% Wt, 3 Eq, 720 μmol) was added. After 30 min, N-methyl-9-(tetrahydro-2H-pyran-2-yl)-9H-purin-6-amine (61.6 mg, 1.1 Eq, 264 μmol) was added. The mixture was stirred at room temperature for 3 hours. LCMS OK. The mixture was quenched by water (3 mL) and extracted with ethyl acetate (3×20 ml), dried over Na₂SO₄ and evaporated in vacuo. The crude residue was purified by flash (Mobile Phase A: Water, Mobile Phase B: ACN; Flow rate: 40 mL/min; Gradient: 10% B to 30% B in 7 min); After solvent evaporation afforded the title compound rel −3-(1-(tert-butyl)-5-(2-(methyl(9-(tetrahydro-2H-pyran-2-yl)-9H-purin-6-yl) amino) acetamido)-1H-pyrazol-3-yl) cyclopentyl isopropylcarbamate (113 mg, 194 μmol, 81.0%) as a white solid. m/z (ES⁺) [M+H]⁺=582.50; HPLC tR=1.042 min.

### rel-(1R,3S)-3-(5-(2-(methyl(9H-purin-6-yl)amino)acetamido)-1H-pyrazol-3-yl)cyclopentyl isopropylcarbamate

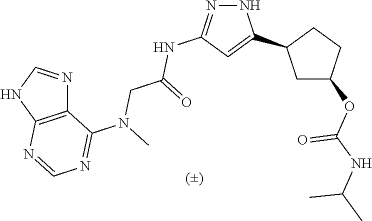 [SMILES: CC(C)NC(=O)O[C@@H]1CC[C@H](c2cc(NC(=O)CN(C)c3ncnc4[nH]cnc34)n[nH]2)C1]

Step 3. The solution of rel −3-(1-(tert-butyl)-5-(2-(methyl(9-(tetrahydro-2H-pyran-2-yl)-9H-purin-6-yl) amino) acetamido)-1H-pyrazol-3-yl) cyclopentyl isopropylcarbamate (5×10 mg, 1 Eq, 86 μmol) in FA (5 xl mL) was heated under 80° C. for 1 hour. After cooling to room temperature, the mixture was concentrated and purified by Prep-HPLC (Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient: 18% B to 22% B in 7 min). After solvent evaporation afforded the title compound rel −3-(5-(2-(methyl(9-(tetrahydro-2H-pyran-2-yl)-9H-purin-6-yl) amino) acetamido)-1H-pyrazol-3-yl) cyclopentyl isopropylcarbamate (25 mg, 48 μmol, 55%) as light yellow solid. m/z (ES⁺) [M+H]⁺=442.20; HPLC tR=0.583 min.

### rel-(1R,3S)-3-(5-(2-(methyl(7H-purin-6-yl)amino)acetamido)-1H-pyrazol-3-yl)cyclopentyl isopropylcarbamate

### rel-(1S,3R)-3-(5-(2-(methyl(7H-purin-6-yl)amino)acetamido)-1H-pyrazol-3-yl)cyclopentyl isopropylcarbamate

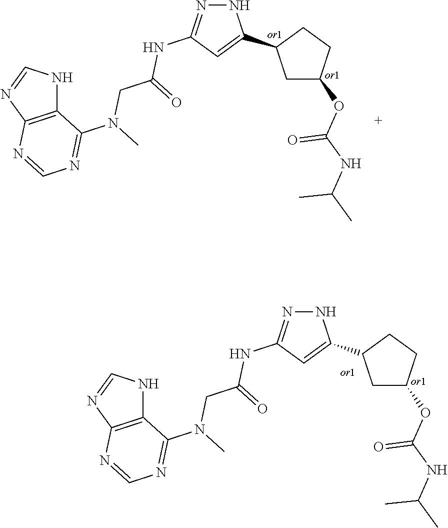

Step 4. rel −3-(5-(2-(methyl(9-(tetrahydro-2H-pyran-2-yl)-9H-purin-6-yl) amino) acetamido)-1H-pyrazol-3-yl) cyclopentyl isopropylcarbamate (25 mg, 57 μmol) was purified by Chiral-HPLC (Column: CHIRALPAK IG, 2*25 cm, 5 m; Mobile Phase A: Hex (0.2% DEA)—HPLC, Mobile Phase B: EtOH: DCM=1:1—HPLC; Flow rate: 20 mL/min; Gradient: 40% B to 40% B in 17 min; Wave Length: 220/254 nm; RT1(min): 11.49; RT2(min):14.64). Lyophilization yielded rel-(1R,3S)-3-(5-(2-(methyl(7H-purin-6-yl) amino) acetamido)-1H-pyrazol-3-yl) cyclopentyl isopropylcarbamate (4.3 mg, 9.7 μmol, 34%) as a white solid. m/z (ES⁺) [M+H]⁺=442.25; HPLC tR=0.709 min.

1H NMR (400 MHz, DMSO) δ 1.00 (dd, J=6.7, 3.9 Hz, 5H), 1.12-1.28 (m, 3H), 1.54-1.69 (m, 4H), 1.82-1.91 (m, 1H), 1.97 (d, J=9.7 Hz, 1H), 2.35 (s, 1H), 3.02 (s, 3H), 3.49-3.58 (m, 1H), 3.72 (s, 2H), 4.49 (s, 1H), 4.97 (s, 1H), 5.11 (s, 2H), 6.25 (s, 1H), 6.91 (d, J=7.9 Hz, 1H), 8.10 (s, 1H), 8.21 (s, 1H), 10.48 (s, 1H), 12.04 (s, 1H), 13.01 (s, 1H).

Lyophilization yielded rel-(1S,3R)-3-(5-(2-(methyl(7H-purin-6-yl) amino) acetamido)-1H-pyrazol-3-yl) cyclopentyl isopropylcarbamate (3.8 mg, 8.6 μmol, 30%) as a white solid.

m/z (ES⁺) [M+H]⁺=442.25; HPLC tR=0.728 min.

1H NMR (400 MHz, DMSO) δ 1.00 (dd, J=6.7, 3.8 Hz, 5H), 1.16-1.28 (m, 4H), 1.54 (s, 1H), 1.67 (t, J=11.4 Hz, 2H), 1.82-1.91 (m, 1H), 1.97 (d, J=9.8 Hz, 1H), 2.39 (s, 1H), 3.03 (d, J=9.1 Hz, 3H), 3.54 (s, 1H), 3.76 (s, 2H), 4.49 (s, 1H), 4.97 (s, 1H), 5.12 (s, 2H), 6.25 (s, 1H), 6.91 (d, J=7.7 Hz, 1H), 8.10 (s, 1H), 8.20 (s, 1H), 10.48 (s, 1H), 12.04 (s, 1H), 13.01 (s, 1H).

### Example 39

### (3R,5R)-5-(3-(1-methyl-3-(2-(trifluoromethoxy)ethyl)-1H-pyrazole-5-carboxamido)-1H-pyrazol-5-yl)tetrahydrofuran-3-yl (1-methylcyclopropyl)carbamate

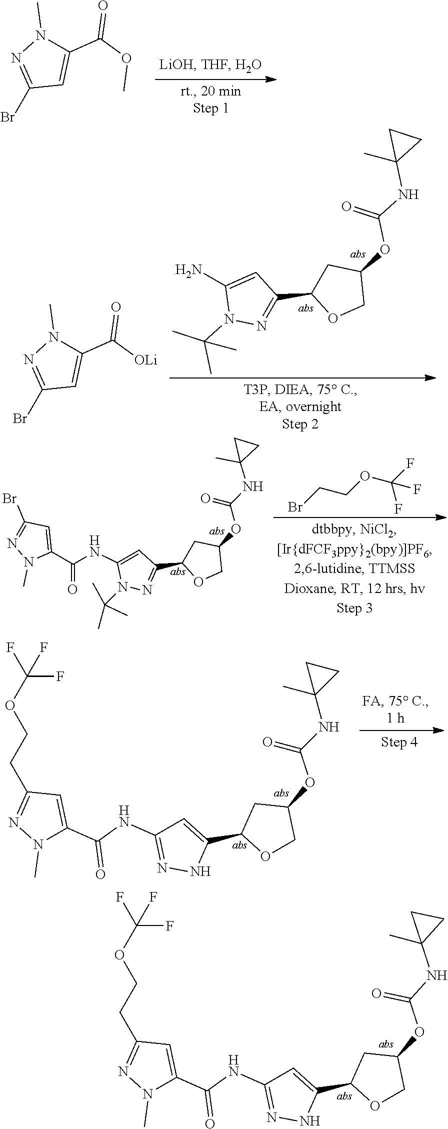

### lithium 3-bromo-1-methyl-1H-pyrazole-5-carboxylate

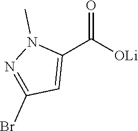

Step 1. To a stirred solution of methyl 3-bromo-1-methyl-1H-pyrazole-5-carboxylate (500 mg, 1 Eq, 2.28 mmol) in THE (5 mL) was added LiOH (104 mg, 4.34 mL, 1 molar, 1.9 Eq, 4.34 mmol) in Water at room temperature under nitrogen atmosphere. The resulting mixture was stirred for 0.5 hour at 50 C. The mixture was concentrated under reduced pressure. The residue was purified by reverse flash chromatography with the following conditions: column, C18 silica gel; mobile phase, MeCN in water, 0% to 10% gradient in 15 min; detector, UV 254 nm, Concentration in vacuo resulted in lithium 3-bromo-1-methyl-1H-pyrazole-5-carboxylate (400 mg, 1.9 mmol, 60%) as white solid. m/z (ES⁺) [M+H]⁺=207.07; HPLC tR=0.992 min.

### (3R,5R)-5-(1-(tert-butyl)-5-(1-methyl-3-((trifluoromethoxy)methyl)-1H-pyrazole-5-carboxamido)-1H-pyrazol-3-yl)tetrahydrofuran-3-yl (1-methylcyclopropyl)carbamate

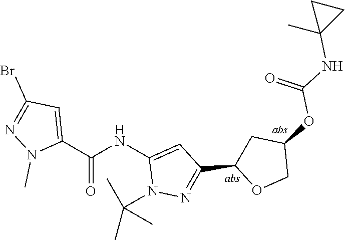 [SMILES: Cn1nc(Br)cc1C(=O)Nc1cc([C@H]2C[C@@H](OC(=O)NC3(C)CC3)CO2)nn1C(C)(C)C]

Step 2. To a solution of lithium 1-methyl-3-((trifluoromethoxy)methyl)-1H-pyrazole-5-carboxylate (25 mg, 0.12 mmol) and (3R,5R)-5-(3-amino-1-(tert-butyl)-1H-pyrazol-5-yl)tetrahydrofuran-3-yl (1-methylcyclopropyl)carbamate (49 mg, 0.15 mmol) in ethyl acetate (1 mL) was added DIEA (160 mg, 1.2 mmol). To above reaction was added T3P (620 mg, 50% Wt in EA, 0.98 mmol) at 0° C. The reaction was stirred at 75° C. for overnight. The mixture was quenched with water, filtered and extracted with EA(3*20 ml). The combined organic layers were washed with brine, dried over sodium sulfate and concentrated. The residue was purified by reverse flash chromatography with the following conditions: column, C18 silica gel; mobile phase, MeCN in water, 60% gradient in 30 min; detector, UV 254 nm to afford (3R,5R)-5-(1-(tert-butyl)-5-(1-methyl-3-((trifluoromethoxy)methyl)-1H-pyrazole-5-carboxamido)-1H-pyrazol-3-yl)tetrahydrofuran-3-yl (1-methylcyclopropyl)carbamate (60 mg, 120 μmol, 97%) as a white solid. m/z (ES⁺) [M+H]⁺=509.37; HPLC tR=1.41 min.

### (3R,5R)-5-(1-(tert-butyl)-3-(1-methyl-3-(2-(trifluoromethoxy)ethyl)-1H-pyrazole-5-carboxamido)-1H-pyrazol-5-yl)tetrahydrofuran-3-yl (1-methylcyclopropyl)carbamate

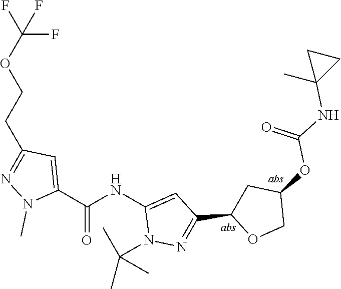 [SMILES: Cn1nc(CCOC(F)(F)F)cc1C(=O)Nc1cc([C@H]2C[C@@H](OC(=O)NC3(C)CC3)CO2)nn1C(C)(C)C]

Step 3. To an 8-mL vial there was added NiCl₂ diglyme (1.1 mg, 4.9 μmol), 4,4-di-tert-butyl-2,2-bipyridyl (1.3 mg, 4.9 μmol), (4,4′-Di-t-butyl-2,2′-bipyridine)bis[3,5-difluoro-2-(5-trifluoromethyl-2-pyridinyl-kN)phenyl-kC]iridium(III) hexafluorophosphate (1.1 mg, 0.98 μmol) and (3R,5R)-5-(3-(3-bromo-1-methyl-1H-pyrazole-5-carboxamido)-1-(tert-butyl)-1H-pyrazol-5-yl)tetrahydrofuran-3-yl (1-methylcyclopropyl)carbamate (50 mg, 98 μmol). Reaction was capped and purged with nitrogen 3 times before dissolving solids in 1,4-Dioxane (1.00 mL). To the solution, 1-bromo-2-(trifluoromethoxy)ethane (38 mg, 26 μL, 0.20 mmol), Tris(trimethylsilyl)silane (24 mg, 30 μL, 1 Eq, 98 μmol), and 2,6-lutidine (32 mg, 34 μL, 3 Eq, 0.29 mmol) were added all while sparging with nitrogen. Reaction was then inserted in the Merck Photoreactor, reacting at 100% light intensity for 150 minutes. Reaction was filtered through a pad of celite then concentrated and redissolved in DMSO and placed on an AccQ prep system. The residue was purified by reverse flash chromatography with the following conditions: column, C18 silica gel; mobile phase, MeCN in water, 60% gradient in 30 min; detector, UV 254 nm to afford. (3R,5R)-5-(1-(tert-butyl)-3-(1-methyl-3-(2-(trifluoromethoxy)ethyl)-1H-pyrazole-5-carboxamido)-1H-pyrazol-5-yl)tetrahydrofuran-3-yl (1-methylcyclopropyl)carbamate (4 mg, 7 μmol, 8%) m/z (ES⁺) [M+H]⁺=543.48; HPLC tR=1.47 min.

### (3R,5R)-5-(3-(1-methyl-3-(2-(trifluoromethoxy)ethyl)-1H-pyrazole-5-carboxamido)-1H-pyrazol-5-yl)tetrahydrofuran-3-yl (1-methylcyclopropyl)carbamate

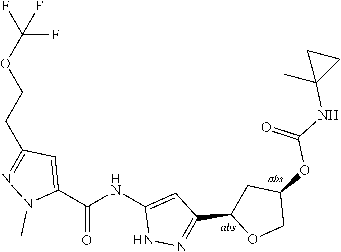 [SMILES: Cn1nc(CCOC(F)(F)F)cc1C(=O)Nc1cc([C@H]2C[C@@H](OC(=O)NC3(C)CC3)CO2)n[nH]1]

Step 4. To the residue of (3R,5R)-5-(1-(tert-butyl)-5-(1-methyl-3—((trifluoronethoxy)methyl)-1H-pyrazole-5-carboxamido)-1H-pyrazol-3-yl)tetrahydrofuran-3-yl (1-methylcyclopropyl)carbamate (5 mg, 7 umol) was added FA (2 mL). The reaction was stirred at 75° C. for 1 h. The mixture was concentrated and purified by reverse flash chromatography with the following conditions: column, C 18 silica gel; mobile phase, MeCN in water, 50% to 60% gradient in 30 min; detector, UV 254 nm. Lyophilization yielded (3R,5R)-5-(3-(1-methyl-3-(2-(trifluoromethoxy)ethyl)-1H-pyrazole-5-carboxamido)-1H-pyrazol-5-yl)tetrahydrofuran-3-yl (1-methylcyclopropyl)carbamate (1.3 mg, 2.7 μmol, 26%, 99.4% Purity) as a white solid. m/z (ES⁺) [M+H]⁺=487.31; HPLC tR=1.288 min.

Additional compounds prepared according to the methods of Example 39 are depicted in Table 14 below.

TABLE 14
Additional Exemplary Compounds

| | | | MS |
|---|---|---|---|
| Compound | Structure | ¹H NMR | [M + 1] |
| (3S,5S)-5-(3- (1-methyl-3-(2- (trifluorometh- oxy)ethyl)-1H- pyrazole-5- carboxamido)- 1H-pyrazol-5- yl)tetrahydro- furan-3-yl (1- methylcyclo- propyl) carbamate | 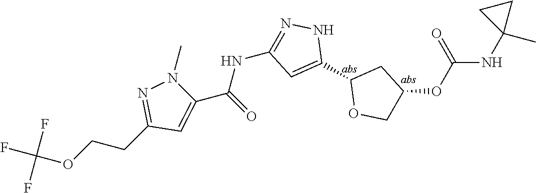 [SMILES: Cn1nc(CCOC(F)(F)F)cc1C(=O)Nc1cc([C@@H]2C[C@H](OC(=O)NC3(C)CC3)CO2)[nH]n1] | (400 MHz, DMSO-d6) 12.51 (s, 1H), 10.81 (s, 1H), 7.52 (s, 1H), 7.05 (s, 1H), 6.54 (s, 1H), 5.19 (d, J = 25.2 Hz, 1H), 5.03-4.78 (m, 1H), 4.31 (t, J = 6.5 Hz, 2H), 4.04 (s, 3H), 3.84 (d, J = 5.4 Hz, 2H), 2.97 (t, J = 6.5 Hz, 2H), 2.70 (dt, J = 14.4, 7.6 Hz, 1H), 1.95 (d, J-9.8 Hz, 1H), 1.25 (s, 3H), 0.62 (d, J = 5.2 Hz, 2H), 0.48 (q, J = 4.7 Hz, 2H). | 487.15 |

### Example 40

### (3R,5R)-5-(3-((2-((S*)-1-methoxyethyl)pyrazolo[1,5-a]pyrazin-4-yl)amino)-1H-pyrazol-5-yl)tetrahydrofuran-3-yl (1-methylcyclopropyl)carbamate

### (3R,5R)-5-(3-((2-((R^{•})-1-methoxyethyl)pyrazolo[1,5-a]pyrazin-4-yl)amino)-1H-pyrazol-5-yl)tetrahydrofuran-3-yl (1-methylcyclopropyl)carbamate

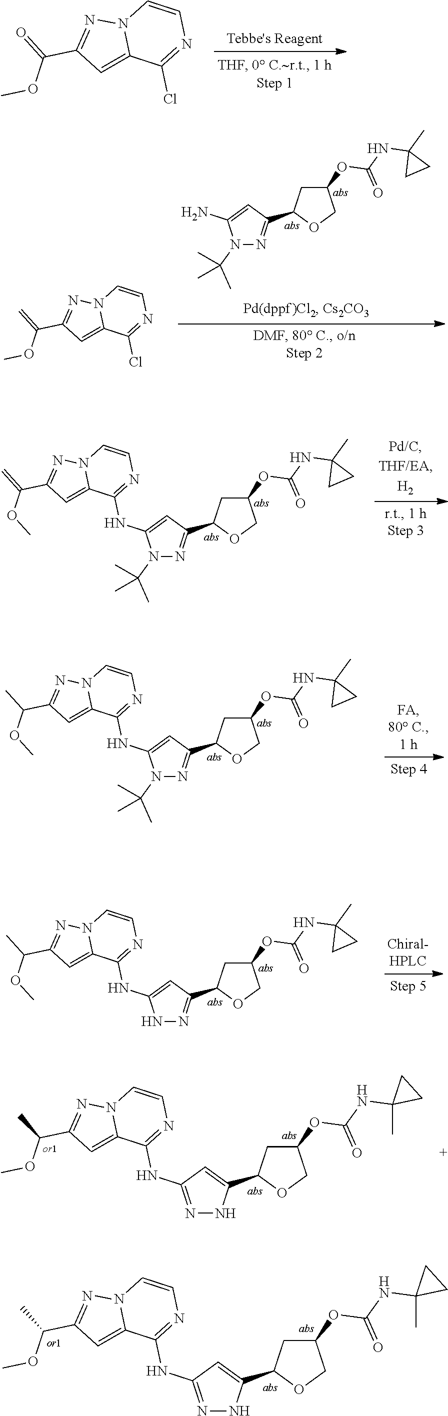

### 4-chloro-2-(1-methoxyvinyl)pyrazolo[1,5-a]pyrazine

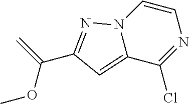

Step 1. A stirred solution of methyl 4-chloropyrazolo[1,5-a]pyrazine-2-carboxylate (1 g, 1 Eq, 5 mmol) in THF (14 mL) was cooled to 0° C. and treated with Tebbe's Reagent (1eq, 9.4 mL, 0.5 M in toluene) under N₂. The reaction was stirred at 0° C. for 30 min. After 30 minutes the solution was warmed to room temperature and stirred for 1 h. The mixture was carefully quenched with (0.1 N) NaOH solution at 0° C. This mixture was treated with hexanes and the solids removed by filtration through a pad of Celite. The solids were washed with hexanes and the filtrate passed through a second pad of Celite to remove any newly formed solids. The organic layer was dried with Na₂SO₄ and concentrated under reduced pressure. The residue was purified by reverse flash chromatography with the following conditions: column, C18 silica gel; mobile phase, MeCN in water, 53% gradient in 8 min; detector, UV 254 nm to afford 4-chloro-2-(1-methoxyvinyl)pyrazolo[1,5-a]pyrazine (460 mg, 2.19 mmol, 50%) as a white solid. .m/z (ES⁺) [M+H]⁺=210.20; HPLC tR=0.887 min.

### (3R,5R)-5-(1-(tert-butyl)-5-((2-(-inethoxyvinyl)pyrazolo[1,5-a]pyrazin-4-yl)amino)-1H-pyrazol-3-yl)tetrahydrofuran-3-yl (1-methylcyclopropyl)carbamate

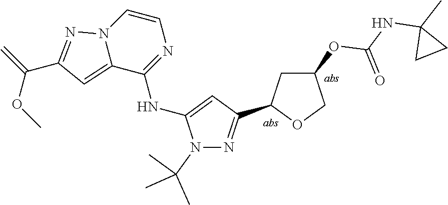 [SMILES: C=C(OC)c1cc2c(Nc3cc([C@H]4C[C@@H](OC(=O)NC5(C)CC5)CO4)nn3C(C)(C)C)nccn2n1]

Step 2, To a stirred solution of 4-chloro-2-(1-methoxyvinyl)pyrazolo[1,5-a]pyrazine (100 mg, 1 Eq, 477 μmol) and (3R,5R)-5-(5-amino-1-(tert-butyl)-1H-pyrazol-3-yl)tetrahydrofuran-3-yl (1-methylcyclopropyl)carbamate (154 mg, 1 Eq, 477 μmol) in DMF (3 mL) was added Cs₂CO₃ (466 mg, 3 Eq, 1.43 mmol) and PdCl₂(dppf)-CH₂Cl2 adduct (77.9 mg, 0.2 Eq, 95.4 μmol) under N₂. The reaction was stirred at 80° C. for overnight. The mixture was diluted with water, and the aqueous phase was extracted with EA. The combined organic layers were washed with brine, dried over sodium sulfate, filtered, and concentrated. The residue was purified by reverse flash chromatography with the following conditions: column, C18 silica gel; mobile phase, MeCN in water, 40% to 50% gradient in 10 min; detector, UV 254 nm to afford (3R,5R)-5-(1-(tert-butyl)-5-((2-(1-methoxyvinyl)pyrazolo[1,5-a]pyrazin-4-yl)amino)-1H-pyrazol-3-yl)tetrahydrofuran-3-yl (1-methylcyclopropyl)carbamate (80 mg, 0.16 mmol, 34%) as colorless oil. m/z (ES⁺) [M+H]⁺=496.20; HPLC tR=0.824 min.

### (3R,5R)-5-(1-(tert-butyl)-5-((2-(1-methoxyethyl)pyrazolo[1,5-a]pyrazin-4-yl)amino)-1H-pyrazol-3-yl)tetrahydrofuran-3-yl (1-methylcyclopropyl)carbamate

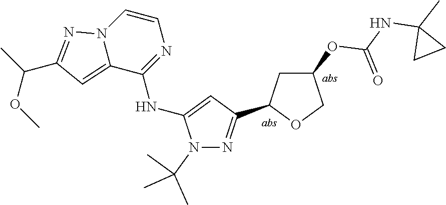 [SMILES: COC(C)c1cc2c(Nc3cc([C@H]4C[C@@H](OC(=O)NC5(C)CC5)CO4)nn3C(C)(C)C)nccn2n1]

Step 3. To a stirred solution of (3R,5R)-5-(1-(tert-butyl)-5-((2-(1-methoxyvinyl)pyrazolo[1,5-a]pyrazin-4-yl)amino)-1H-pyrazol-3-yl)tetrahydrofuran-3-yl (1-methylcyclopropyl)carbamate (230 mg, 1 Eq, 464 μmol) in THE (5 mL) and Ethyl acetate (5 mL) was added Pd/C(49.4 mg, 1 Eq, 464 μmol) under N₂. The reaction was stirred at room temperature for 1 h under H₂. The mixture was filtered and concentrated to afford (3R,5R)-5-(1-(tert-butyl)-5-((2-(1-methoxyethyl)pyrazolo[1,5-a]pyrazin-4-yl)amino)-1H-pyrazol-3-yl)tetrahydrofuran-3-yl (1-methylcyclopropyl)carbamate (160 mg, 322 μmol, 69.3%) as colorless oil. m/z (ES⁺) [M+H]⁺=498.25; HPLC tR=0.746 min.

### (3R,5R)-5-(5-((2-(1-methoxyethyl)pyrazolo[1,5-a]pyrazin-4-yl)amino)-1Hpyrazol-3-yl)tetrahydrofuran-3-yl (1-methylcyclopropyl)carbamate

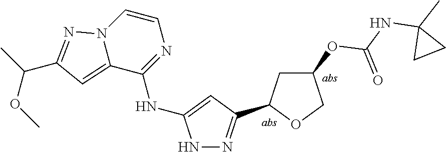 [SMILES: COC(C)c1cc2c(Nc3cc([C@H]4C[C@@H](OC(=O)NC5(C)CC5)CO4)n[nH]3)nccn2n1]

Step 4. To the (3R,5R)-5-(1-(tert-butyl)-5-((2-(1-methoxyethyl)pyrazolo[1,5-a]pyrazin-4-yl)amino)-1H-pyrazol-3-yl)tetrahydrofuran-3-yl (1-methylcyclopropyl)carbamate (160 mg, 1 Eq, 322 μmol) was added FA (5 mL). The reaction was stirred at 80° C. for 1 h. The mixture was concentrated and purified by reverse flash chromatography with the following conditions: column, C18 silica gel; mobile phase, MeCN in water, 51% gradient in 8 min; detector, UV 254 nm to afford (3R,5R)-5-(5-((2-(1-methoxyethyl)pyrazolo[1,5-a]pyrazin-4-yl)amino)-1Hpyrazol-3-yl)tetrahydrofuran-3-yl (1-methylcyclopropyl)carbamate (90 mg, 0.20 mmol, 63%) as colorless oil. m/z (ES⁺) [M+H]⁺=442.35; HPLC tR=0.603 min.

### (3R,5R)-5-(3-((2-((S*)-1-methoxyethyl)pyrazolo[1,5-a]pyrazin-4-yl)amino)-1H-pyrazol-5-yl)tetrahydrofuran-3-yl (1-methylcyclopropyl)carbamate

### (3R,5R)-5-(3-((2-((R*)-1-methoxyethyl)pyrazolo[1,5-a]pyrazin-4-yl)amino)-11-pyrazol-5-yl)tetrahydrofuran-3-yl (1-methylcyclopropyl)carbamate

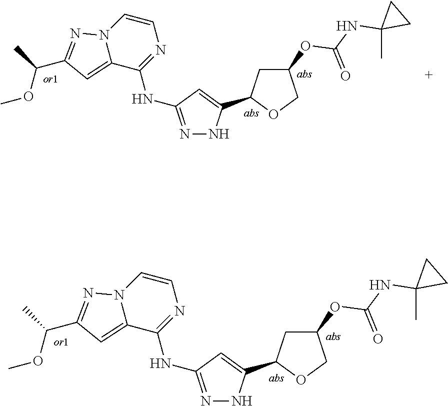 [SMILES: CO[C@@H](C)c1cc2c(Nc3cc([C@H]4C[C@@H](OC(=O)NC5(C)CC5)CO4)[nH]n3)nccn2n1 ; CO[C@H](C)c1cc2c(Nc3cc([C@H]4C[C@@H](OC(=O)NC5(C)CC5)CO4)[nH]n3)nccn2n1]

Step 5. The (3R,5R)-5-(5-((2-(1-methoxyethyl)pyrazolo[1,5-a]pyrazin-4-yl)amino)-1H-pyrazol-3-yl)tetrahydrofuran-3-yl (1-methylcyclopropyl)carbamate (90 mg, 1 Eq, 0.20 mmol) was subjected to Prep-Chiral-HPLC (Column: CHIRALPAK IG, 2*25 cm, 5 μm; Mobile Phase A: Hex(0.5% 2M NH3-MeOH)—HPLC, Mobile Phase B: EtOH: DCM=1:1—HPLC; Flow rate: 20 mL/min: Gradient: 50% B to 50% B in 13 min; Wave Length: 220/254 nm; RT1(min): 6.04; Sample Solvent: EtOH: DCM=1:1—HPLC; Injection Volume: 0.8 mL; Number Of Runs: 3). Lyophilization yielded (3R,5R)-5-(3-((2-((S*)-1-methoxyethyl)pyrazolo[1,5-a]pyrazin-4-yl)amino)-1H-pyrazol-5-yl)tetrahydrofuran-3-yl (1-methylcyclopropyl)carbamate (28.9 mg, 64.7 μmol, 64%, 98.9% Purity) as a white solid. m/z (ES⁺) [M+H]⁺=442.15; HPLC tR=1.377 min. 1H NMR (400 MHz, DMSO-d6) 12.36 (s, 1H), 10.00 (s, 1H), 8.05 (s, 1H), 7.53 (s, 1H), 7.41-7.23 (m, 2H), 6.79 (s, 1H), 5.17 (s, 1H), 4.84 (s, IH), 4.55 (q, J=6.5 Hz, 1H), 3.85 (s, 2H), 3.22 (s, 3H), 2.68 (s, 1H), 1.93 (d, J=15.3 Hz, 1H), 1.47 (d, J=6.5 Hz, 3H), 1.25 (s, 3H), 0.61 (s, 2H), 0.48 (q, J=4.6 Hz, 2H).

The (3R,5R)-5-(5-((2-(1-methoxyethyl)pyrazolo[1,5-a]pyrazin-4-yl)amino)-1H-pyrazol-3-yl)tetrahydrofuran-3-yl (1-methylcyclopropyl)carbamate (90 mg, 1 Eq, 0.20 mmol) was subjected to Prep-Chiral-HPLC (Column: CHIRALPAK IG, 2*25 cm, 5 μm; Mobile Phase A: Hex(0.5% 2M NH3-MeOH)—HPLC, Mobile Phase B: EtOH: DCM=1:1—HPLC; Flow rate: 20 mL/min; Gradient: 50% B to 50% B in 13 min; Wave Length: 220/254 nm; RT2(min): 9.67; Sample Solvent: EtOH: DCM=1:1—HPLC; Injection Volume: 0.8 mL; Number Of Runs: 3). Lyophilization yielded (3R,5R)-5-(3-((2-((R*)-1-methoxyethyl)pyrazolo[1,5-a]pyrazin-4-yl)amino)-1H-pyrazol-5-yl)tetrahydrofuran-3-yl (1-methylcyclopropyl)carbamate (31.1 mg, 63.1 μmol, 62%, 89.6% Purity) as a white amorphous solid. m/z (ES⁺) [M+H]⁺=442.15; HPLC tR=1.377 min. ¹H NMR (400 MHz, DMSO-d6) 12.36 (s, 1H), 10.18 (d, J=131.9 Hz, 1H), 8.05 (s, 1H), 7.53 (s, 1H), 7.42-7.17 (m, 2H), 6.79 (s, 1H), 5.17 (s, 1H), 4.84 (s, 1H), 4.55 (q, J=6.5 Hz, 1H), 3.85 (s, 2H), 3.22 (s, 3H), 2.71 (s, 1H), 1.96 (s, 1H), 1.47 (d, J=6.5 Hz, 3H), 1.25 (s, 3H), 0.60 (d, J=5.1 Hz, 2H), 0.48 (q, J=4.5 Hz, 2H).

### Example 41

### (3R,5R)-5-(3-((2-(trifluoromethyl)imidazo[1,2-c]pyrimidin-5-yl)amino)-1H-pyrazol-5-yl)tetrahydrofuran-3-yl (1-methylcyclopropyl)carbamate

### (3R,5S)-5-(3-((2-(trifluoromethyl)imidazo[1,2-c]pyrimidin-5-yl)amino)-1H-pyrazol-5-yl)tetrahydrofuran-3-yl (1-methylcyclopropyl)carbamate

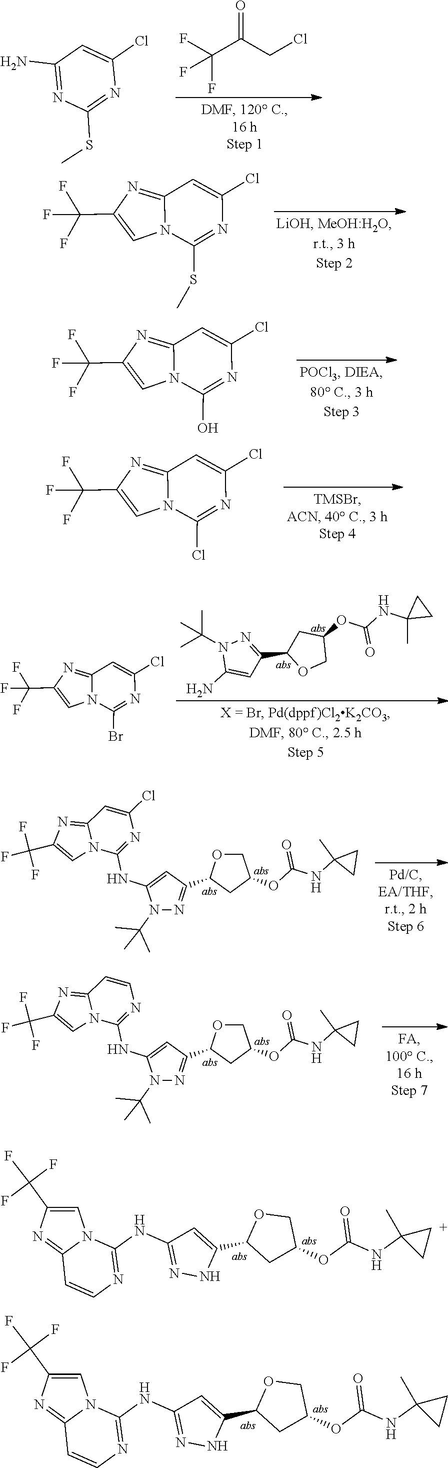

### 7-chloro-5-(methylthio)-2-(trifluoromethyl)imidazo[1,2-c]pyrimidine

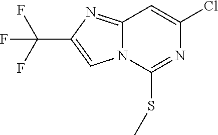 [SMILES: CSc1nc(Cl)cc2nc(C(F)(F)F)cn12]

Step 1. A round bottomed flask was charged with 6-chloro-2-(methylthio)pyrimidin-4-amine (5 g, 1 Eq, 0.03 mol), DMF (20 mL), 3-chloro-1,1,1-trifluoropropan-2-one (6 g, 1.5 Eq, 0.04 mol) was added, and the solution was stirred at 120° C. for 16 hours. The residue was concentrated in vacuo and purified by reverse flash chromatography with the following conditions: column, C18 silica gel; mobile phase, (acetonitrile/water), 0% to 100% gradient in 45 min; detector, UV 254 nm. Concentration in vacuo resulted in 7-chloro-5-(methylthio)-2-(trifluoromethyl)imidazo[1,2-c]pyrimidine (3.4 g, 13 mmol, 40%) as a yellow amorphous solid. m/z (ES⁺) [M+H]⁺=267.85; HPLC tR=0.875 min.

### 7-chloro-2-(trifluoromethyl)imidazo[1,2-c]pyrimidin-5-ol

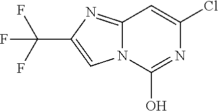 [SMILES: Oc1nc(Cl)cc2nc(C(F)(F)F)cn12]

Step 2. A round bottomed flask was charged with 7-chloro-5-(methylthio)-2-(trifluoromethyl)imidazo[1,2-c]pyrimidine (3.4 g, 1 Eq, 13 mmol), MeOH:H₂O=2:1 (20 mL), LiOH (1.2 g, 4 Eq, 51 mmol) was added, and the solution was stirred at 25° C. for 3 hours. The reaction mixture was adjusted the pH value to 7-8 with 1 N HCl solution. The residue was concentrated in vacuo and diluted with water (15 mL), and the aqueous phase was extracted with EA (50 mL) three times. The combined organic layers were washed with brine, dried over sodium sulfate, filtered. Concentration in vacuo resulted in 7-chloro-2-(trifluoromethyl)imidazo[1,2-c]pyrimidin-5-ol (2.9 g, 12 mmol, 96%) as a brown amorphous solid. m/z (ES⁺) [M+H]⁺=237.90; HPLC tR=0.700 min.

### 5,7-dichloro-2-(trifluoromethyl)imidazo[1,2-c]pyrimidine

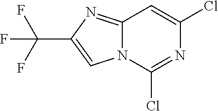 [SMILES: FC(F)(F)c1cn2c(Cl)nc(Cl)cc2n1]

Step 3. A round bottomed flask was charged with 7-chloro-2-(trifluoromethyl)imidazo[1,2-c]pyrimidin-5-ol (1 g, 1 Eq, 4 mmol), POCl₃ (15 mL), DIEA (3 g, 4 mL, 5 Eq, 0.02 mol) was added, and the solution was stirred at 80° C. for 3 hours. The solution was concentrated in vacuo and quenched with NaHCO₃ solution (0° C.) and adjust the pH value to 7-8 with NaHCO₃. The aqueous phase was extracted with EA (40 mL) three times. The combined organic layers were washed with brine, dried over sodium sulfate, filtered. Concentration in vacuo resulted in 5,7-dichloro-2-(trifluoromethyl)imidazo[1,2-c]pyrimidine (840 mg, 3.28 mmol, 80%) as a brown oil. m/z (ES⁺) [M+H]⁺=255.90; HPLC tR=0.842 min.

### 5-bromo-7-chloro-2-(trifluoromethyl)imidazo[1,2-c]pyrimidine

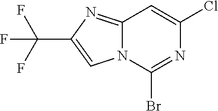 [SMILES: FC(F)(F)c1cn2c(Br)nc(Cl)cc2n1]

Step 4. A round bottomed flask was charged with 5,7-dichloro-2-(trifluoromethyl)imidazo[1,2-c]pyrimidine (700 mg, 1 Eq, 2.73 mmol), ACN (10 mL), TMSBr (837 mg, 2 Eq, 5.47 mmol) was added, and the solution was stirred at 40° C. for 3 hour. The crude product was purified by silica gel chromatography (10 g column; eluting with PE/EA; ratio:15/1). Concentration in vacuo resulted in 5-bromo-7-chloro-2-(trifluoromethyl)imidazo[1,2-c]pyrimidine (720 mg, 2.40 mmol, 87.6%) as a brown amorphous solid. m/z (ES⁺) [M+H]⁺=299.80; HPLC tR=0.867 min.

### (3R,5R)-5-(1-(tert-butyl)-5-((7-chloro-2-(trifluoromethyl)imidazo[1,2-c]pyrimidin-5-yl)amino)-1H-pyrazol-3-yl)tetrahydrofuran-3-yl (1-methylcyclopropyl)carbamate

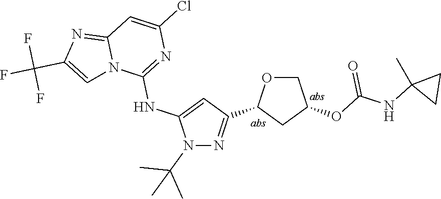 [SMILES: CC1(NC(=O)O[C@H]2CO[C@@H](c3cc(Nc4nc(Cl)cc5nc(C(F)(F)F)cn45)n(C(C)(C)C)n3)C2)CC1]

Step 5. A resealable reaction vial was charged with 5-bromo-7-chloro-2-(trifluoromethyl)imidazo[1,2-c]pyrimidine (550 mg, 1 Eq, 1.83 mmol), DMF(5 mL), (3R,5R)-5-(5-amino-1-(tert-butyl)-1H-pyrazol-3-yl)tetrahydrofuran-3-yl (1-methylcyclopropyl)carbamate (885 mg, 1.5 Eq, 2.75 mmol), potassium carbonate (1.01 g, 4 Eq, 7.32 mmol) and PdCl₂(dppf) (201 mg, 0.15 Eq, 275 μmol) was added. The resulting mixture was stirred at 80° C. for 2.5 hours under nitrogen atmosphere. The residue was concentrated in vacuo and purified by reverse flash chromatography with the following conditions: column, C18 silica gel; mobile phase, (acetonitrile/water), 0% to 100% gradient in 45 min; detector, UV 254 nm. Lyophilization yielded resulted in (3R,5R)-5-(1-(tert-butyl)-5-((7-chloro-2-(trifluoromethyl)imidazo[1,2-c]pyrimidin-5-yl)amino)-1H-pyrazol-3-yl)tetrahydrofuran-3-yl (1-methylcyclopropyl)carbamate (295 mg, 0.33 mmol, 18%, 60% Purity) as a yellow amorphous solid. m/z (ES⁺) [M+H]⁺=542.05; HPLC tR=0.900 min.

### (3R,5R)-5-(3-((2-(trifluoromethyl)imidazo[1,2-c]pyrimidin-5-yl)amino)-1H-pyrazol-5-yl)tetrahydrofuran-3-yl (1-methylcyclopropyl)carbamate (3R,5S)-5-(3-((2-(trifluoromethyl)imidazo[1,2-c]pyrimidin-5-yl)amino)-1H-pyrazol-5-yl)tetrahydrofuran-3-yl (1-methylcyclopropyl)carbamate

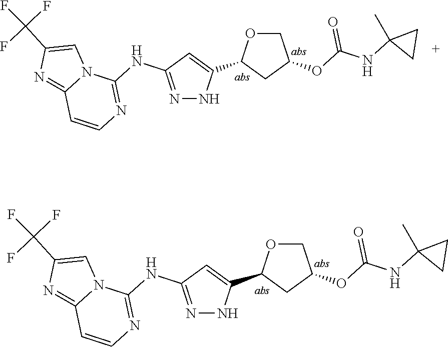 [SMILES: CC1(NC(=O)O[C@H]2CO[C@@H](c3cc(Nc4nccc5nc(C(F)(F)F)cn45)n[nH]3)C2)CC1 ; CC1(NC(=O)O[C@H]2CO[C@H](c3cc(Nc4nccc5nc(C(F)(F)F)cn45)n[nH]3)C2)CC1]

Step 6. A round bottomed flask was charged with (3R,5R)-5-(1-(tert-butyl)-5-((2-(trifluoromethyl)imidazo[1,2c]pyrimidin-5-yl)amino)-1H-pyrazol-3-yl)tetrahydrofuran-3-yl (1-methylcyclopropyl)carbamate (140 mg, 1 Eq, 276 μmol), FA (6 mL) was added, and the solution was stirred at 100° C. for 16 hours. The residue was concentrated in vacuo and purified by Pre-HPLC (Column: Xselect CSH C18 OBD Column 30*150 mm 5 μm, n; Mobile Phase A: Water(0.1% FA), Mobile Phase B: ACN; Flow rate: 60 mL/min; Gradient: 32% B to 44% B in 8 min, 44% B; Wave Length: 254; 220 nm; RT1(min): 6.5, 7.48(min)). Lyophilization yielded resulted in (3R,5R)-5-(3-((2-(trifluoromethyl)imidazo[1,2-c]pyrimidin-5-yl)amino)-1H-pyrazol-5-yl)tetrahydrofuran-3-yl (1-methylcyclopropyl)carbamate (1.9 mg, 4.2 μmol, 1.5%, m/z (ES⁺) [M+H]⁺=452.15; HPLC tR=1.178 min) as an off-white amorphous solid and (3R,5S)-5-(3-((2-(trifluoromethyl)imidazo[1,2-c]pyrimidin-5-yl)amino)-1H-pyrazol-5-yl)tetrahydrofuran-3-yl (1-methylcyclopropyl)carbamate (2 mg, 4 μmol, 2%, m/z (ES⁺) [M+H]⁺=452.15; HPLC tR=1.258 min) as a white amorphous solid.

### Example 42

### (3R,5R)-5-(3-(1-methyl-3-(trifluoromethoxy)-1H-pyrazole-5-carboxamido)-1H-pyrazol-5-yl)tetrahydrofuran-3-yl (1-methylcyclopropyl)carbamate

### (3R,5R)-5-(3-(3-(chlorodifluoromethoxy)-1-methyl-1H-pyrazole-5-carboxamido)-1H-pyrazol-5-yl)tetrahydrofuran-3-yl (1-methylcyclopropyl)carbamate

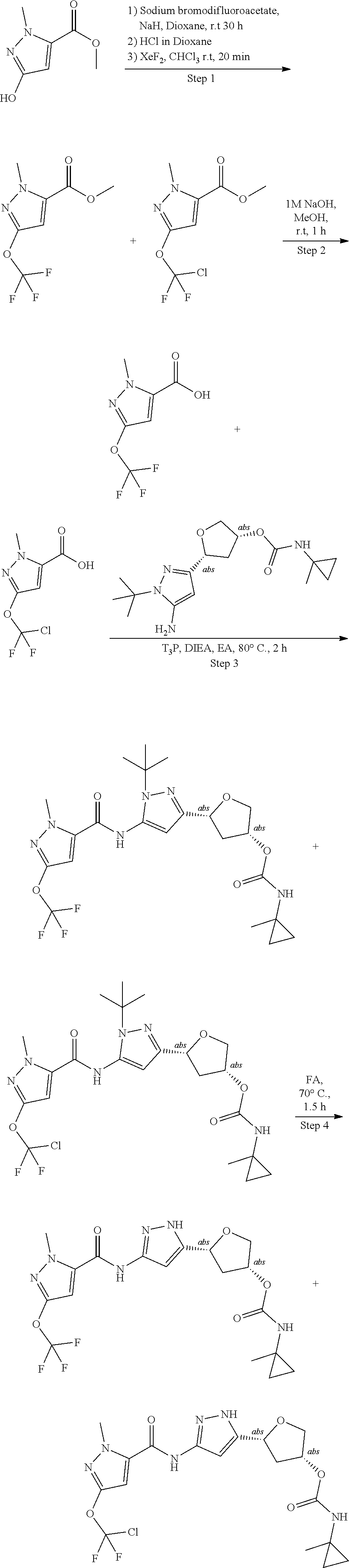

### methyl 3-(bromodifluoromethoy)-1-methyl-1-H-pyrazole-5-carboxylate

### methyl 3-(chlorodifluoromethoxy)-1-methyl-1 H-pyrazole-5-carboylate

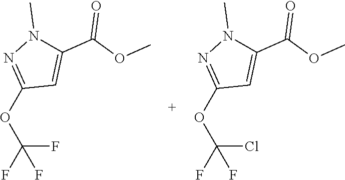

Step 1. To a mixture of methyl 3-hydroxy-1-methyl-1H-pyrazole-5-carboxylate (400 mg, 2.56 mmol), Dioxane (5 mL) was added NaH (0.18 g, 7.69 mmol) in portions at 25° C. under nitrogen atmosphere. The mixture was stirred for 30 min at 25° C. prior addition of 2-bromo-2,2-difluoroacetic acid sodium salt (608 mg, 3.07 mmol). The mixture was stirred for 30 h at 25° C. The solid was filtered out. The filtrate was concentrated under vacuum. HCl in Dioxane (5 mL) was added, and concentrated in vacuo. DCM (5 mL) and XeF₂ (1.31 g, 7.69 mmol) were added, and the mixture was stirred for 30 min at 25° C. The reaction mixture was diluted with H₂O (50 mL), and the aqueous phase was extracted with EA (100 mL) three times. The combined organic layers were washed with brine, dried over sodium sulfate, filtered. Concentration in vacuo resulted in a mixture of methyl 3-(bromodifluoromethoxy)-1-methyl-1H-pyrazole-5-carboxylate and methyl 3-(chlorodifluoromethoxy)-1-methyl-1H-pyrazole-5-carboxylate (180 mg, 26.8%) as a yellow oil.

methyl 3-(bromodifluoromethoxy)-1-methyl-1H-pyrazole-5-carboxylate: m/z (ES⁺) [M+H]⁺=224.95; HPLC tR=0.875 min.

methyl 3-(chlorodifluoromethoxy)-1-methyl-1H-pyrazole-5-carboxylate: m/z (ES⁺) [M+H]⁺=240.90; HPLC tR=0.875 min.

### 1-methyl-3-(trifluoromethoxy)-1H-pyrazole-5-carboxylic acid

### 3-(chlorodifluoromethoxy)-1-methyl-1H-pyrazole-5-carboxylic acid

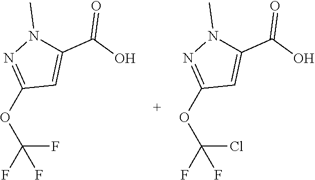

Step 2. A resealable reaction vial was charged with a mixture of methyl 1-methyl-3-(trifluoromethoxy)-1H-pyrazole-5-carboxylate and methyl 3-(chlorodifluoromethoxy)-1-methyl-1H-pyrazole-5-carboxylate (160 mg, 0.70 mmol), NaOH (0.86 mL, 0.86 mmol), MeOH (5 mL) and a stirbar before being evacuated and purged with nitrogen three times, and the mixture was stirred for 1 h at 25° C. The reaction mixture was diluted with H₂O (20 mL), and the aqueous phase was extracted with EA (20 mL) three times. then adjusted to pH 1-3 with 1M HCl, and the aqueous phase was extracted with EA (30 mL) three times. The combined organic layers were washed with brine, dried over sodium sulfate, filtered and concentrated in vacuo. Concentration in vacuo resulted in a mixture of 1-methyl-3-(trifluoromethoxy)-1H-pyrazole-5-carboxylic acid and 3-(chlorodifluoromethoxy)-1-methyl-1H-pyrazole-5-carboxylic acid (120 mg, 80%) as a colourless oil.

1-methyl-3-(trifluoromethoxy)-1H-pyrazole-5-carboxylic acid: m/z (ES⁺) [M+H]⁺=210.95; HPLC tR=0.750 min.

3-(chlorodifluoromethoxy)-1-methyl-1H-pyrazole-5-carboxylic acid: m/z (ES⁺) [M+H]⁺=226.90; HPLC tR=0.750 min.

### (3R,5R)-5-(1-(tert-butyl)-5-(1-methyl-3-(trifluoromethoxy)-1H-pyrazole-5-carboxamido)-1H-pyrazol-3-yl)tetrahydrofuran-3-yl (1-methylcyclopropyl)carbamate

### (3R,5R)-5-(1-(tert-butyl)-5-(3-(chlorodifluoromethoxy)-1-methyl-1H-pyrazole-5-carboxamido)-1H-pyrazol-3-yl)tetrahydrofuran-3-yl (1-methylcyclopropyl)carbamate

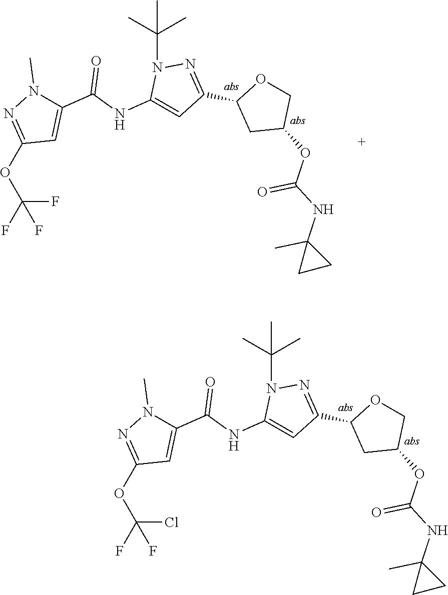 [SMILES: Cn1nc(OC(F)(F)F)cc1C(=O)Nc1cc([C@H]2C[C@@H](OC(=O)NC3(C)CC3)CO2)nn1C(C)(C)C ; Cn1nc(OC(F)(F)Cl)cc1C(=O)Nc1cc([C@H]2C[C@@H](OC(=O)NC3(C)CC3)CO2)nn1C(C)(C)C]

Step 3. To a mixture of 1-methyl-3-(trifluoromethoxy)-1H-pyrazole-5-carboxylic acid and 3-(chlorodifluoromethoxy)-1-methyl-1H-pyrazole-5-carboxylic acid (109 mg, 0.50 mmol), DIEA (0.41 g, 0.55 mL, 3.1 mmol) and (3R,5R)-5-(5-amino-1-(tert-butyl)-1H-pyrazol-3-yl)tetrahydrofuran-3-yl (1-methylcyclopropyl)carbamate (0.10 g, 0.31 mmol) in EA (5 mL) was added T3P (1.60 g, 50% wt, 2.5 mmol in EA) drop wise at 0° C. under nitrogen atmosphere. The mixture was stirred for 2 h at 80° C. The reaction mixture was diluted with H₂O (50 mL), and the aqueous phase was extracted with EA (100 mL) three times. The combined organic layers were washed with brine, dried over sodium sulfate, filtered. Concentration in vacuo resulted in a mixture of (3R,5R)-5-(1-(tert-butyl)-5-(1-methyl-3-(trifluoromethoxy)-1H-pyrazole-5-carboxamido)-1H-pyrazol-3-yl)tetrahydrofuran-3-yl (1-methylcyclopropyl)carbamate and (3R,5R)-5-(1-(tert-butyl)-5-(3-(chlorodifluoromethoxy)-1-methyl-1H-pyrazole-5-carboxamido)-1H-pyrazol-3-yl)tetrahydrofuran-3-yl (1-methylcyclopropyl)carbamate (300 mg, crude) as a yellow oil.

(3R,5R)-5-(1-(tert-butyl)-5-(1-methyl-3-(trifluoromethoxy)-1H-pyrazole-5-carboxamido)-1H-pyrazol-3-yl)tetrahydrofuran-3-yl (1-methylcyclopropyl)carbamate: m/z (ES⁺) [M+H]⁺=515.15; HPLC tR=1.158 min.

(3R,5R)-5-(1-(tert-butyl)-5-(3-(chlorodifluoromethoxy)-1-methyl-1H-pyrazole-5-carboxamido)-1H-pyrazol-3-yl)tetrahydrofuran-3-yl (1-methylcyclopropyl)carbamate: m/z (ES⁺) [M+H]⁺=531.10; HPLC tR=1.178 min.

### (3R,5R)-5-(3-(1-methyl-3-(trifluoromethoxy)-1H-pyrazole-5-carboxamido)-1H-pyrazol-5-yl)tetrahydrofuran-3-yl (1-methylcyclopropyl)carbamate

### (3R,5R)-5-(3-(3-(chlorodifluoromethoxy)-1-methyl-1H-pyrazole-5-carboxamido)-1H-pyrazol-5-yl)tetrahydrofuran-3-yl (1-methylcyclopropyl)carbamate

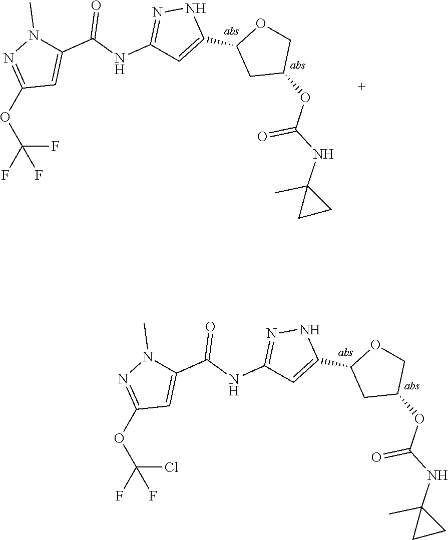 [SMILES: Cn1nc(OC(F)(F)F)cc1C(=O)Nc1cc([C@H]2C[C@@H](OC(=O)NC3(C)CC3)CO2)[nH]n1 ; Cn1nc(OC(F)(F)Cl)cc1C(=O)Nc1cc([C@H]2C[C@@H](OC(=O)NC3(C)CC3)CO2)[nH]n1]

Step 4. A resealable reaction vial was charged with a mixture of (3R,5R)-5-(1-(tert-butyl)-5-(1-methyl-3-(trifluoromethoxy)-1H-pyrazole-5-carboxamido)-1H-pyrazol-3-yl)tetrahydrofuran-3-yl (1-methylcyclopropyl)carbamate and (3R,5R)-5-(1-(tert-butyl)-5-(3-(chlorodifluoromethoxy)-1-methyl-1H-pyrazole-5-carboxamido)-1H-pyrazol-3-yl)tetrahydrofuran-3-yl (1-methylcyclopropyl)carbamate (190 mg,crude), FA (5 mL) was added, and a stirbar before being evacuated and purged with nitrogen three times, and the mixture was stirred for 1.5 h at 70° C. The reaction concentrated in vacuo. The resulting crude material was purified by Pre-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 55% B in 7 min, 55% B; Wave Length: 220 nm; RT1(min): 7.27). Lyophilization yielded (3R,5R)-5-(3-(1-methyl-3-(trifluoromethoxy)-1H-pyrazole-5-carboxamido)-1H-pyrazol-5-yl)tetrahydrofuran-3-yl (1-methylcyclopropyl)carbamate (38.6 mg, 22.8%) as a white amorphous solid. m/z (ES⁺) [M+H]⁺=459.15; HPLC tR=1.318 min. ¹H NMR (400 MHz, DMSO-d6) δ 12.57 (s, 1H), 7.52 (s, 1H), 7.12 (s, 1H), 6.54 (s, 1H), 5.16 (s, 1H), 4.84 (t, J=7.9 Hz, 1H), 4.06 (s, 3H), 3.84 (d, J=4.6 Hz, 2H), 2.70 (dt, J=14.4, 7.5 Hz, 1H), 1.93 (t, J=10.3 Hz, 1H), 1.25 (s, 3H), 0.61 (d, J=5.1 Hz, 2H), 0.48 (q, J=4.6 Hz, 2H).

A resealable reaction vial was charged with a mixture of (3R,5R)-5-(1-(tert-butyl)-5-(1-methyl-3-(trifluoromethoxy)-1H-pyrazole-5-carboxamido)-1H-pyrazol-3-yl)tetrahydrofuran-3-yl (1-methylcyclopropyl)carbamate and (3R,5R)-5-(1-(tert-butyl)-5-(3-(chlorodifluoromethoxy)-1-methyl-1H-pyrazole-5-carboxamido)-1H-pyrazol-3-yl)tetrahydrofuran-3-yl (1-methylcyclopropyl)carbamate (190 mg,crude), FA (5 mL) was added, and a stirbar before being evacuated and purged with nitrogen three times, and the mixture was stirred for 1.5 h at 70° C. The reaction concentrated in vacuo. The resulting crude material was purified by Pre-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 25% B to 55% B in 7 min, 55% B; Wave Length: 220 nm; RT1(min): 7.27). Lyophilization yielded (3R,5R)-5-(3-(3-(chlorodifluoromethoxy)-1-methyl-1H-pyrazole-5-carboxamido)-1H-pyrazol-5-yl)tetrahydrofuran-3-yl (1-methylcyclopropyl)carbamate (50 mg, crude) as a white amorphous solid. The resulting crude material was purified by Pre-HPLC (Column: XBridge Prep OBD C18 Column, 30*150 mm, 5 μm; Mobile Phase A: Water (10 mmol/L NH₄HCO₃), Mobile Phase B: MeOH—HPLC; Flow rate: 60 mL/min; Gradient: 49% B to 67% B in 8 min, 67% B; Wave Length: 254 nm; RT1(min): 7.85). Lyophilization yielded (3R,5R)-5-(3-(3-(chlorodifluoromethoxy)-1-methyl-1H-pyrazole-5-carboxamido)-1H-pyrazol-5-yl)tetrahydrofuran-3-yl (1-methylcyclopropyl)carbamate (9.3 mg, 21%) as a white amorphous solid. m/z (ES⁺) [M+H]⁺=475.15; HPLC tR=0.876 min. ¹H NMR (400 MHz, DMSO-d6) δ 12.57 (s, 1H), 11.03 (s, 1H), 7.51 (s, 1H), 7.17 (s, 1H), 6.56 (s, 1H), 5.16 (s, 1H), 4.85 (s, 1H), 4.07 (s, 3H), 3.85 (s, 2H), 2.70 (dt, J=14.0, 7.2 Hz, 1H), 1.93 (s, 1H), 1.25 (s, 3H), 0.62 (s, 2H), 0.48 (s, 2H).

Additional compounds prepared according to the methods of Example 42 are depicted in Table 15 below.

TABLE 15
Additional Exemplary Compounds

| | | | MS |
|---|---|---|---|
| Compound | Structure | ¹H NMR | [M + 1] |
| (3S,5S)-5-(3-(3- (chlorodifluoro- methoxy)-1- methyl-1H- pyrazole-5- carboxamido)- 1H-pyrazol-5- yl)tetrahydro- furan-3-yl (1- methylcyclo- propyl) carbamate | 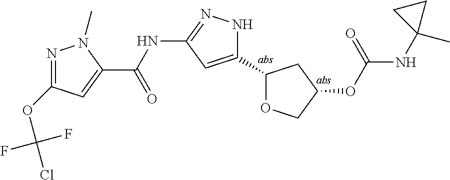 [SMILES: Cn1nc(OC(F)(F)Cl)cc1C(=O)Nc1cc([C@@H]2C[C@H](OC(=O)NC3(C)CC3)CO2)[nH]n1] | (400 MHz, DMSO-d6) 12.57 (s, 1H), 11.02 (s, 1H), 7.51 (s, 1H), 7.17 (s, 1H), 6.57 (s, 1H), 5.16 (s, 1H), 4.93 (d, J = 57.3 Hz, 1H), 4.07 (s, 3H), 3.85 (s, 2H), 2.71 (dd, J = 14.1, 7.3 Hz, 1H), 1.93 (s, 1H), 1.25 (s, 3H), 0.62 (s, 2H), 0.48 (q, J = 5.0, 4.6 Hz, 2H). | 475.25 |

### Example 43

### (3R,5R)-5-(3-(1-methyl-3-((S*)-2,2,2-trifuoro-1-methoxyethyl)-1H-pyrazole-5-carboxamido)-1H-pyrazol-5-yl)tetrahydrofuran-3-yl (1-methylcyclopropyl)carbamate

### (3R,5R)-5-(3-(1-methyl-3-((R*)-2,2,2-trifuoro-1-methoxyethyl)-1H-pyrazole-5-carboxamido)-1H-pyrazol-5-yl)tetrahydrofuran-3-yl (1-methylcyclopropy)carbamate

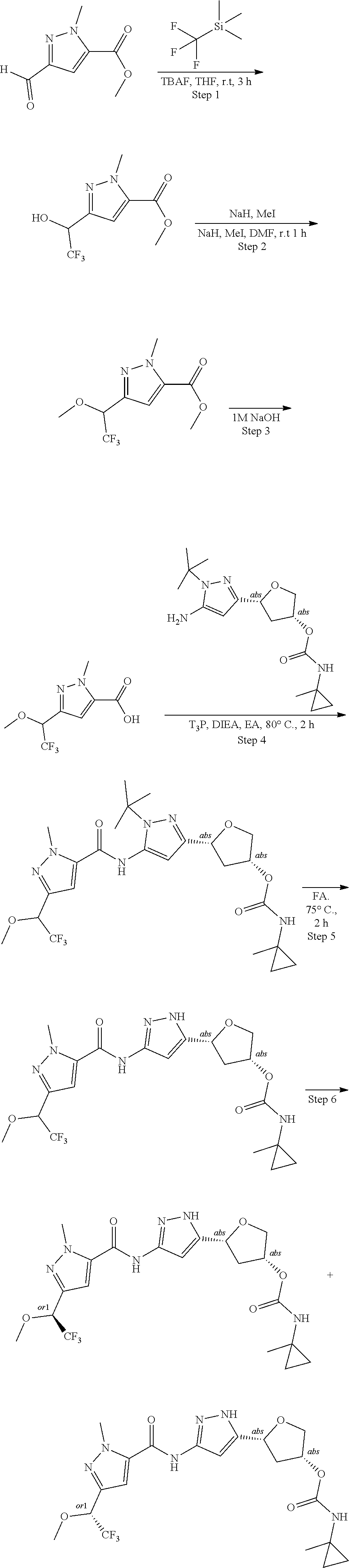

### methyl 1-methyl-3-(2,2,2-trifluoro-1-hydroxyethyl)-1H-pyrazole-5-carboxylate

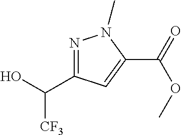 [SMILES: COC(=O)c1cc(C(O)C(F)(F)F)nn1C]

Step 1. A resealable reaction vial was charged with methyl 3-formyl-1-methyl-1H-pyrazole-5-carboxylate (500 mg, 2.97 mmol), trimethyl(trifluoromethyl)silane (719 mg, 5.06 mmol), TBAF (155 mg, 0.60 mmol), THF (5 mL) was added, and a stirbar before being evacuated and purged with nitrogen three times, and the mixture was stirred for 3 h at 25° C. The reaction concentrated in vacuo. The residue was purified by reverse flash chromatography with the following conditions: column, C18 silica gel; mobile phase, MeCN in water, 10% to 50% gradient in 10 min; detector, UV 254 nm. Concentration in vacuo resulted in methyl 1-methyl-3-(2,2,2-trifluoro-1-hydroxyethyl)-1H-pyrazole-5-carboxylate (400 mg, crude) as a colorless oil. m/z (ES⁺) [M+H]⁺=239.20; HPLC tR=0.720 min.

### methyl 1-methyl-3-(2,2,2-trifluoro-1-methoxyethyl)-1H-pyrazole-5-carboxylate

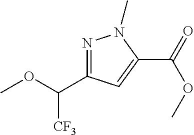 [SMILES: COC(=O)c1cc(C(OC)C(F)(F)F)nn1C]

Step 2. To a mixture of methyl 1-methyl-3-(2,2,2-trifluoro-1-hydroxyethyl)-1H-pyrazole-5-carboxylate (390 mg, 1.64 mmol) in DMF (5 mL) was added NaH (0.12 g, 4.91 mmol) in portions at 0° C. under nitrogen atmosphere. The mixture was stirred for 5 min at 0° C. prior addition of iodomethane (697 mg, 4.91 mmol). The mixture was stirred for 1 h at 25° C. The reaction solution is directly used for the next step. m/z (ES⁺) [M+H]⁺=253.00; HPLC tR=0.788 min.

### 1-methyl-3-(2,2,2-trifluoro-1-methoxyethyl)-1H-pyrazole-5-carboxylic acid

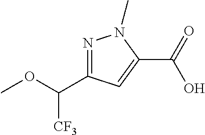 [SMILES: COC(c1cc(C(=O)O)n(C)n1)C(F)(F)F]

Step 3. Above reaction solution was added NaOH (3.27 mL, 3.27 mmol) and stirred for 1 h at 25° C. The reaction mixture was diluted with H₂O (20 mL), and the aqueous phase was extracted with EA (30 mL) three times. then adjusted to pH 1-3 with 1 M HCl. and the aqueous phase was extracted with EA (30 mL) three times. The combined organic layers were washed with brine, dried over sodium sulfate, filtered and concentrated in vacuo. Concentration in vacuo resulted in 1-methyl-3-(2,2,2-trifluoro-1-methoxyethyl)-1H-pyrazole-5-carboxylic acid (180 mg, 46.2%) as a colorless oil. m/z (ES⁺) [M+H]⁺=239.20; HPLC tR=0.720 min.

### (3R,5R)-5-(1-(tert-butyl)-5-(1-methyl-3-(2,2,2-trifluoro-1-methoxyethyl)-1H-pyrazole-5-carboxamido)-1H-pyrazol-3-yl)tetrahydrofuran-3-yl (1-methylcyclopropyl)carbamate

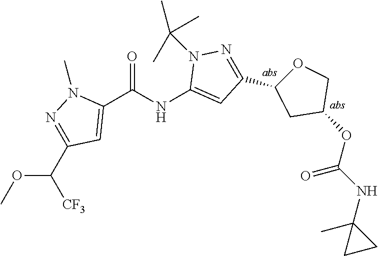 [SMILES: COC(c1cc(C(=O)Nc2cc([C@H]3C[C@@H](OC(=O)NC4(C)CC4)CO3)nn2C(C)(C)C)n(C)n1)C(F)(F)F]

Step 4. To a mixture of 1-methyl-3-(2,2,2-trifluoro-1-methoxyethyl)-1H-pyrazole-5-carboxylic acid (160 mg, 0.67 mmol), DIEA (722 mg, 5.58 mmol) and (3R,5R)-5-(5-amino-1-(tert-butyl)-1H-pyrazol-3-yl)tetrahydrofuran-3-yl (1-methylcyclopropyl)carbamate (180 mg, 0.56 mmol) in EA (5 mL) was added T3P (6.39 g, 10.00 mmol) dropwise at 0° C. under nitrogen atmosphere. The mixture was stirred for 2 h at 80° C. The reaction mixture was diluted with H₂O (30 mL), and the aqueous phase was extracted with EA (20 mL) three times. The combined organic layers were washed with brine, dried over sodium sulfate, filtered and concentrated in vacuo. Concentration in vacuo resulted in (3R,5R)-5-(1-(tert-butyl)-5-(1-methyl-3-(2,2,2-trifluoro-1-methoxyethyl)-1H-pyrazole-5-carboxamido)-1H-pyrazol-3-yl)tetrahydrofuran-3-yl (1-methylcyclopropyl)carbamate (400 mg, crude) as a yellow oil. m/z (ES⁺) [M+H]⁺=543.45; HPLC tR=0.945 min.

### (3R,5R)-5-(3-(1-methyl-3-(2,2,2-trifluoro-1-methoxyethyl)-1H-pyrazole-5-carboxamido)-1H-pyrazol-5-yl)tetrahydrofuran-3-yl (1-methylcyclopropyl)carbamate

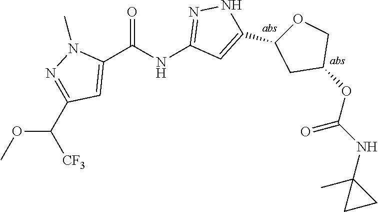 [SMILES: COC(c1cc(C(=O)Nc2cc([C@H]3C[C@@H](OC(=O)NC4(C)CC4)CO3)[nH]n2)n(C)n1)C(F)(F)F]

Step 5. A resealable reaction vial was charged with (3R,5R)-5-(1-(tert-butyl)-5-(1-methyl-3-(2,2,2-trifluoro-1-methoxyethyl)-1H-pyrazole-5-carboxamido)-1H-pyrazol-3-yl)tetrahydrofuran-3-yl (1-methylcyclopropyl)carbamate (380 mg, 0.70 mmol), FA (5 mL) and a stirbar before being evacuated and purged with nitrogen three times, and the mixture was stirred for 2 h at 75° C. The reaction concentrated in vacuo. The resulting crude material was purified by Pre-HPLC (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 26% B to 44% B in 8 min, 44% B; Wave Length: 220 nm; RT1(min): 7.35). Lyophilization yielded (3R,5R)-5-(3-(1-methyl-3-(2,2,2-trifluoro-1-methoxyethyl)-1H-pyrazole-5-carboxamido)-1H-pyrazol-5-yl)tetrahydrofuran-3-yl (1-methylcyclopropyl)carbamate (190 mg, 55.8%) as a white solid. m/z (ES⁺) [M+H]⁺=487.30; HPLC tR=0.820 min.

### (3R,5R)-5-(3-(1-methyl-3-((S*)-2,2,2-trifluoro-1-methoxyethyl)-1H-pyrazole-5-carboxamido)-1H-pyrazol-5-yl)tetrahydrofuran-3-yl (1-methylcyclopropyl)carbamate

### (3R,5R)-5-(3-(1-methyl-3-((R*)-2,2,2-trifuoro-1-methoxyethyl)-1H-pyrazole-5-carboxamido)-1H-pyrazol-5-yl)tetrahydrofuran-3-yl (1-methylcyclopropyl)carbamate

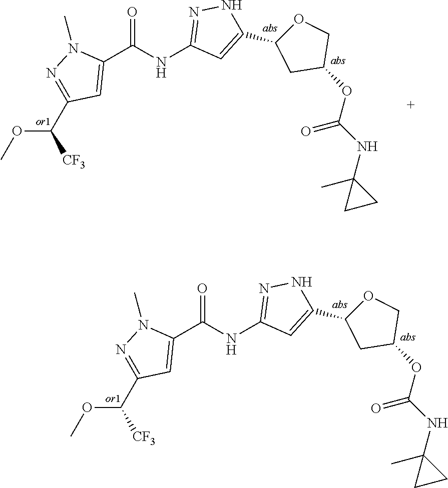 [SMILES: CO[C@@H](c1cc(C(=O)Nc2cc([C@H]3C[C@@H](OC(=O)NC4(C)CC4)CO3)[nH]n2)n(C)n1)C(F)(F)F ; CO[C@H](c1cc(C(=O)Nc2cc([C@H]3C[C@@H](OC(=O)NC4(C)CC4)CO3)[nH]n2)n(C)n1)C(F)(F)F]

Step 6. (3R,5R)-5-(3-(1-methyl-3-(2,2,2-trifluoro-1-methoxyethyl)-1H-pyrazole-5-carboxamido)-1H-pyrazol-5-yl)tetrahydrofuran-3-yl (1-methylcyclopropyl)carbamate (189 mg, 0.39 mmol) was purified by chiral Pre-HPLC (Column: CHIRALPAK IG, 2*25 cm, 5 μm; Mobile Phase A: Hex(0.5% 2M NH₃-MeOH)—HPLC, Mobile Phase B: EtOH: DCM=1:1—HPLC; Flow rate: 20 mL/min; Gradient: 40% B to 40% B in 10 min; Wave Length: 220/254 nm; RT1(min): 5.27; RT2(min): 7.46; Sample Solvent: EtOH: DCM=1:1-HPLC; Injection Volume: 0.45 mL; Number Of Runs: 5). Lyophilization yielded (3R,5R)-5-(3-(1-methyl-3-((S*)-2,2,2-trifluoro-1-methoxyethyl)-1H-pyrazole-5-carboxamido)-1H-pyrazol-5-yl)tetrahydrofuran-3-yl (1-methylcyclopropyl)carbamate (47.5 mg, 50.3%) as an amorphous white solid. m/z (ES⁺) [M+H]⁺=487.20; HPLC tR=0.948 min. ¹H NMR (400 MHz, DMSO-d6) δ 12.50 (s, 1H), 10.94 (s, 1H), 7.50 (s, 1H), 7.30 (s, 1H), 6.56 (s, 1H), 5.16 (s, 1H), 5.03 (q, J=6.9 Hz, 1H), 4.85 (s, 1H), 4.11 (s, 3H), 3.85 (s, 2H), 3.35 (s, 3H), 2.71 (dd, J=14.3, 7.5 Hz, 1H), 1.93 (s, 1H), 1.25 (s, 3H), 0.62 (s, 2H), 0.48 (q, J=5.1, 4.7 Hz, 2H)

(3R,5R)-5-(3-(1-methyl-3-(2,2,2-trifluoro-1-methoxyethyl)-1H-pyrazole-5-carboxamido)-1H-pyrazol-5-yl)tetrahydrofuran-3-yl (1-methylcyclopropyl)carbamate (189 mg, 0.39 mmol) was purified by chiral Pre-HPLC (Column: CHIRALPAK IG, 2*25 cm, 5 μm; Mobile Phase A: Hex(0.5% 2M NH₃-MCOH)—HPLC, Mobile Phase B: EtOH: DCM=1:1—HPLC; Flow rate: 20 mL/min; Gradient: 40% B to 40% B in 10 min; Wave Length: 220/254 nm; RT1(min): 5.27; RT2(min): 7.46; Sample Solvent: EtOH: DCM=1:1-HPLC; Injection Volume: 0.45 mL; Number Of Runs: 5). Lyophilization yielded (3R,5R)-5-(3-(1-methyl-3-((R*)-2,2,2-trifluoro-1-methoxyethyl)-1H-pyrazole-5-carboxamido)-1H-pyrazol-5-yl)tetrahydrofuran-3-yl (1-methylcyclopropyl)carbamate (38.6 mg, 40.8%) as an amorphous white solid. m/z (ES⁺) [M+H]⁺=487.20; HPLC tR=0.948 min. ¹H NMR (400 MHz, DMSO-d6) δ 12.50 (s, 1H), 10.94 (s, 1H), 7.50 (s, 1H), 7.30 (s, 1H), 6.56 (s, 1H), 5.16 (s, 1H), 5.03 (q, J=7.0 Hz, 1H), 4.85 (s, 1H), 4.11 (s, 3H), 3.85 (s, 2H), 3.35 (s, 3H), 2.71 (dd, J=14.4, 7.4 Hz, 1H), 1.93 (s, 1H), 1.25 (s, 3H), 0.62 (s, 2H), 0.55 (d, J=13.0 Hz, 1H), 0.48 (q, J=5.1, 4.7 Hz, 2H).

### Example 44

### (3R,5R)-5-(3-(3-(methoxymethyl)-1-(trifluoromethyl)-1 H-pyrazole-5-carboxamido)-1 H-pyrazol-5-yl)tetrahydrofuran-3-yl (1-methylcyclopropyl)carbamate

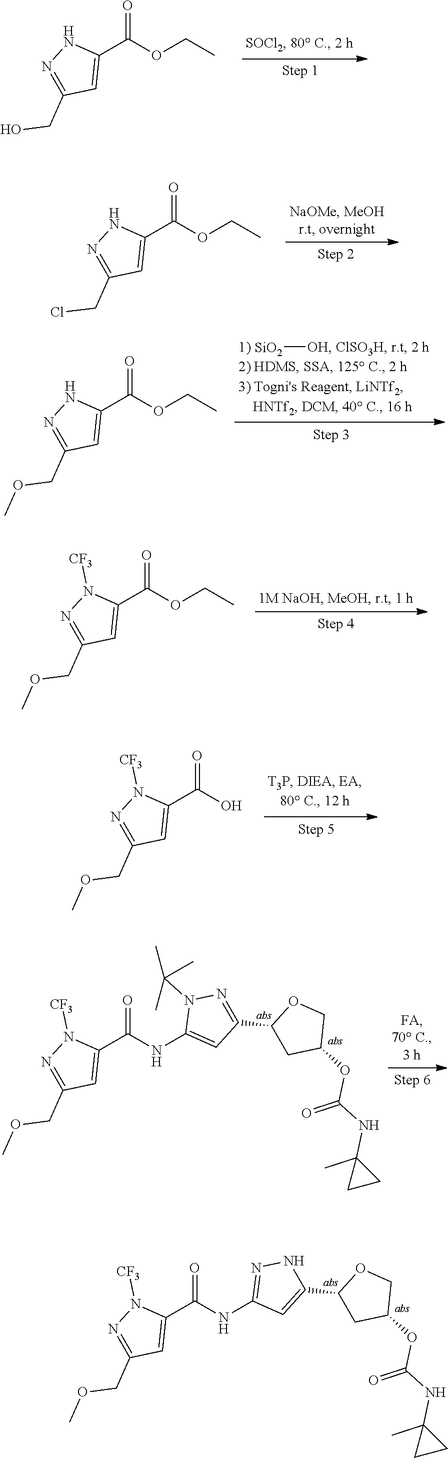

### ethyl 3-(chloromethyl)-1 H-pyrazole-5-carboxylate

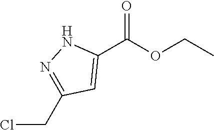

Step 1. A resealable reaction vial was charged with ethyl 3-(hydroxymethyl)-1H-pyrazole-5-carboxylate (2.00 g, 0.01 mol), SOCl₂ (20 mL) was added, and a stirbar before being evacuated and purged with nitrogen three times, and the mixture was stirred for 2 h at 80° C. The reaction concentrated in vacuo. The residue was diluted with water, then adjusted to pH 6-7 with sodium bicarbonate. The aqueous phase was extracted with EA (100 mL) three times. The combined organic layers were washed with brine, dried over sodium sulfate, filtered. Concentration in vacuo resulted in ethyl 3-(chloromethyl)-1H-pyrazole-5-carboxylate (2.20 g, crude) as a colorless oil. m/z (ES⁺) [M+H]⁺=189.20; HPLC tR=0.645 min.

### ethyl 3-(methoxymethyl)-1H-pyrazole-5-carboxylate

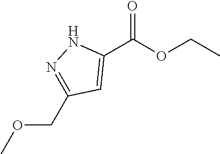

Step 2. To a mixture of ethyl 3-(chloromethyl)-1H-pyrazole-5-carboxylate (2.20 g, 12.00 mmol) in MeOH (20 mL) was added NaOMe (0.82 g, 15.00 mmol) in portions at 0° C. under nitrogen atmosphere. The mixture was stirred for 12 h at 25° C. The reaction concentrated in vacuo. The residue was purified by reverse flash chromatography with the following conditions: column, C18 silica gel; mobile phase, MeCN in water, 10% to 50% gradient in 10 min; detector, UV 254 nm. Concentration in vacuo resulted in ethyl 3-(methoxymethyl)-1H-pyrazole-5-carboxylate (1.60 g, 74%) as a colorless oil. m/z (ES⁺) [M+H]⁺=185.05; HPLC tR=0.633 min.

### ethyl 3-(methoxymethyl)-1-(trifluoromethyl)-1H-pyrazole-5-carboxylate

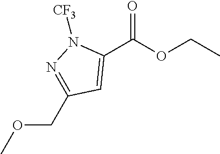 [SMILES: CCOC(=O)c1cc(COC)nn1C(F)(F)F]

Step 3. SiO2-OH (34 mg, 0.42 mmol) was added ClSO₃H (49 mg, 0.42 mmol) dropwise at 25° C. under nitrogen atmosphere. The mixture was stirred for 2 h at 25° C. A resealable reaction vial was charged with ethyl 3-(methoxymethyl)-1H-pyrazole-5-carboxylate (600 mg, 3.26 mmol), HDMS (10 mL) and a stirbar before being evacuated and purged with nitrogen three times, and the mixture was stirred for 2 h at 125° C. The solid was filtered out. The filtrate was concentrated under vacuum. DCM (10 mL) was added, then 3,3-dimethyl-1-(trifluoromethyl)-1,3-dihydro-113-benzo[d][1,2]iodaoxole (1.40 g, 4.20 mmol), LiNTf₂ (0.15 g, 0.51 mmol) and HNTf₂ (0.14 g, 0.51 mmol) were added before being evacuated and purged with nitrogen three times, and the mixture was stirred for 16 h at 40° C. The reaction concentrated in vacuo. The residue was purified by reverse flash chromatography with the following conditions: column, C18 silica gel; mobile phase, MeCN in water, 10% to 50% gradient in 10 min; detector, UV 254 nm. Concentration in vacuo resulted in ethyl 3-(methoxymethyl)-1-(trifluoromethyl)-1H-pyrazole-5-carboxylate (140 mg, crude) as a colorless oil. m/z (ES⁺) [M+H]⁺=253.005; HPLC tR=0.867 min.

### 3-(methoxymethyl)-1-(trifluoromethyl)-1H-pyrazole-5-carboxylic acid

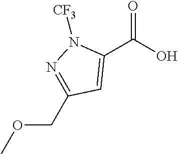

Step 4. A resealable reaction vial was charged with ethyl 3-(methoxymethyl)-1-(trifluoromethyl)-1H-pyrazole-5-carboxylate (180 mg, 0.21 mmol), NaOH (428 pL, 428 μmol), MeOH (5 mL) and a stirbar before being evacuated and purged with nitrogen three times, and the mixture was stirred for 1 h at 25° C. The reaction mixture was diluted with H₂O (20 mL), and the aqueous phase was extracted with EA (20 mL) three times, then adjusted to pH 1-3 with 1M HCl. The aqueous phase was extracted with EA (20 mL) three times. The combined organic layers were washed with brine, dried over sodium sulfate, filtered. Concentration in vacuo resulted in 3-(methoxymethyl)-1-(trifluoromethyl)-1H-pyrazole-5-carboxylic acid (50 mg, crude) as a colorless oil. m/z (ES⁺) [M+H]⁺=225.05; HPLC tR=0.567 min.

### (3R,5R)-5-(1-(tert-butyl)-5-(3-(methoxymethyl)-1-(trifluoromethyl)-1H-pyrazole-5-carboxamido)-1H-pyrazol-3-yl)tetrahydrofuran-3-yl (1-methylcyclopropyl)carbamate

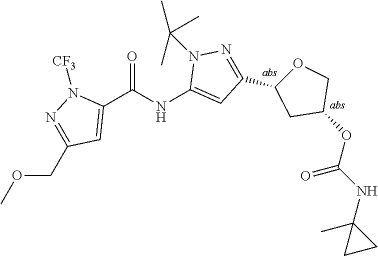 [SMILES: COCc1cc(C(=O)Nc2cc([C@H]3C[C@@H](OC(=O)NC4(C)CC4)CO3)nn2C(C)(C)C)n(C(F)(F)F)n1]

Step 5. To a mixture of 3-(methoxymethyl)-1-(trifluoromethyl)-1H-pyrazole-5-carboxylic acid (119 mg, 0.53 mmol), DIEA (625 mg, 4.84 mmol) and (3R,5R)-5-(5-amino-1-(tert-butyl)-1H-pyrazol-3-yl)tetrahydrofuran-3-yl (1-methylcyclopropyl)carbamate (156 mg, 0.48 mmol) in EA (5 mL) was added T₃P (4.92 g, 50% wt 3.87 mmol in EA) dropwise at 0° C. under nitrogen atmosphere. The mixture was stirred for 12 h at 80° C. The reaction mixture was diluted with H₂O (20 mL), and the aqueous phase was extracted with EA (20 mL) three times. The combined organic layers were washed with brine, dried over sodium sulfate, filtered. Concentration in vacuo resulted in (3R,5R)-5-(1-(tert-butyl)-5-(3-(methoxymethyl)-1-(trifluoromethyl)-1H-pyrazole-5-carboxamido)-1H-pyrazol-3-yl)tetrahydrofuran-3-yl (1-methylcyclopropyl)carbamate (200 mg, 78.2%) as a yellow oil. m/z (ES⁺) [M+H]⁺=529.10; HPLC tR=0.850 min.

### (3R,5R)-5-(3-(3-(methoxymethyl)-1-(trifluoromethyl)-1H-pyrazole-5-carboxamido)-1H-pyrazol-5-yl)tetrahydrofuran-3-yl (1-methylcyclopropyl)carbamate

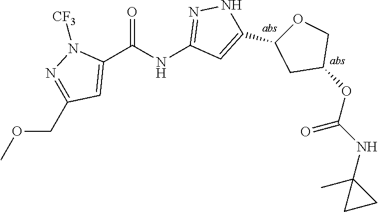 [SMILES: COCc1cc(C(=O)Nc2cc([C@H]3C[C@@H](OC(=O)NC4(C)CC4)CO3)[nH]n2)n(C(F)(F)F)n1]

Step 6. A resealable reaction vial was charged with (3R,5R)-5-(1-(tert-butyl)-5-(3-(methoxymethyl)-1-(trifluoromethyl)-1H-pyrazole-5-carboxamido)-1H-pyrazol-3-yl)tetrahydrofuran-3-yl (1-methylcyclopropyl)carbamate (250 mg, 0.47 mmol), FA (5 mL), and a stirbar before being evacuated and purged with nitrogen three times, and the mixture was stirred for 3 h at 70° C. The reaction concentrated in vacuo. The resulting crude material was purified by Pre-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 20% B to 45% B in 7 min, 45% B; Wave Length: 220 nm; RT1(min): 7.63). Lyophilization yielded (3R,5R)-5-(3-(3-(methoxymethyl)-1-(trifluoromethyl)-1H-pyrazole-5-carboxamido)-1H-pyrazol-5-yl)tetrahydrofuran-3-yl (1-methylcyclopropyl)carbamate (86.8 mg, 38.8%) as an off white solid. m/z (ES⁺) [M+H]⁺=473.15; HPLC tR=0.903 min. ¹H NMR (400 MHz, DMSO-d6) δ 12.58 (s, 1H), 11.36 (s, 1H), 7.52 (s, 1H), 7.17 (s, 1H), 6.55 (s, 1H), 5.16 (s, 1H), 4.85 (s, 1H), 4.47 (s, 2H), 3.85 (s, 2H), 3.33 (s, 3H), 2.74-2.66 (m, 1H), 1.92 (s, 1H), 1.24 (s, 3H), 0.67-0.43 (m, 4H).

### Example 45

### (3R,5R)-5-(3-(1-(2-(trifluoromethoxy)ethyl)-1H-pyrazole-4-carboxamido)-1H-pyrazol-5-yl)tetrahydrofuran-3-yl (1-methylcyclopropyl)carbamate

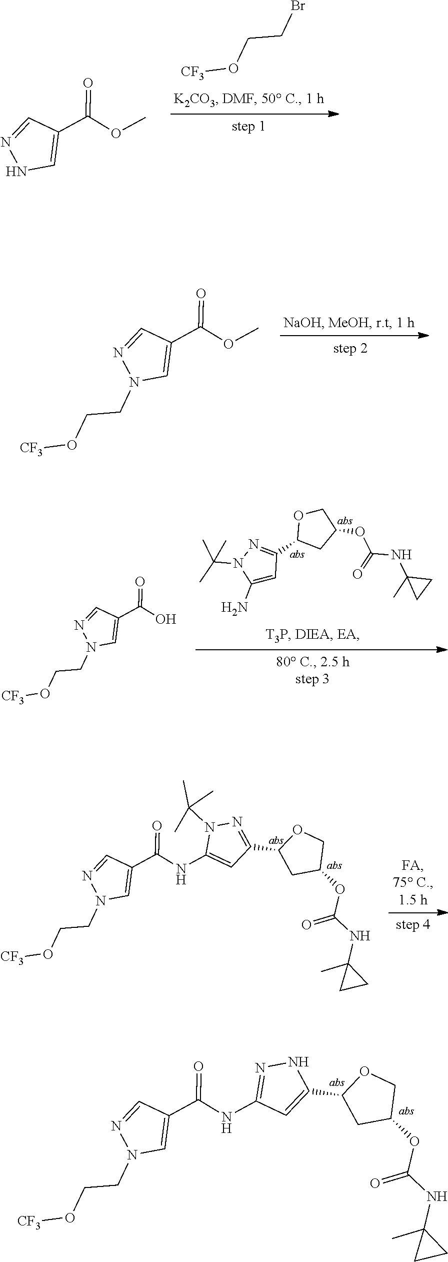

### methyl 1-(2-(trifluoromethoxy)ethyl)-1H-pyrazole-4-carboxylate

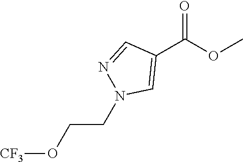 [SMILES: COC(=O)c1cnn(CCOC(F)(F)F)c1]

Step 1. To a mixture of methyl 1H-pyrazole-4-carboxylate (110 mg, 0.87 mmol) in DMF (5 mL) was added 1-bromo-2-(trifluoromethoxy)ethane (252 mg, 1.31 mmol) and K2C03 (362 mg, 2.62 mmol). The mixture was stirred for 1 hour at 50° C. The reaction mixture was diluted with H₂O (15 mL), and the aqueous phase was extracted with EA (30 mL) three times. The combined organic layers were washed with brine, dried over sodium sulfate, filtered. Concentration in vacuo resulted in methyl 1-(2-(trifluoromethoxy)ethyl)-1H-pyrazole-4-carboxylate (170 mg, 81.8%) as a white solid. m/z (ES⁺) [M+H]⁺=238.95; HPLC tR=0.742 min. 54s

### 1-(2-(trifluoromethoxy)ethyl)-1H-pyrazole-4-carboxylic acid

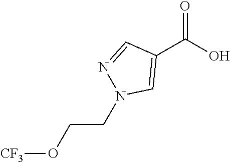 [SMILES: O=C(O)c1cnn(CCOC(F)(F)F)c1]

Step 2. A resealable reaction vial was charged with methyl 1-(2-(trifluoromethoxy)ethyl)-1H-pyrazole-4-carboxylate (160 mg, 0.67 mmol) and NaOH (54 mg, 1.34 mmol), MeOH (4 mL), and a stirbar before being evacuated and purged with nitrogen three times, and the mixture was stirred for 1 hour at 25° C. The reaction mixture was diluted with H₂O (15 mL), and the aqueous phase was extracted with EA (30 mL) three times. The pH value of the aqueous layer was adjusted to 1-3 with 1 M HCl. The aqueous layer was extracted with 3×30 mL of ethyl acetate. The organic layers were combined, washed with brine, dried. Concentration in vacuo resulted in 1-(2-(trifluoromethoxy)ethyl)-1H-pyrazole-4-carboxylic acid (100 mg, 66.4%) as a white solid. m/z (ES⁺) [M+H]⁺=224.95; HPLC tR=0.458 min.

### (3R,5R)-5-(1-(tert-butyl)-5-(1-(2-(trifluoromethoxy)ethyl)-1H-pyrazole-4-carboxamido)-1H-pyrazol-3-yl)tetrahydrofuran-3-yl (1-methylcyclopropyl)carbamate

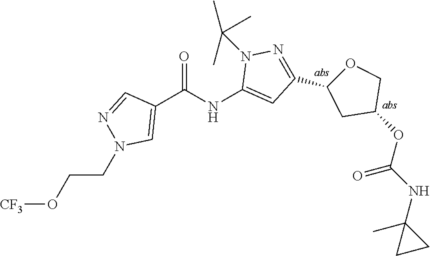 [SMILES: CC1(NC(=O)O[C@H]2CO[C@@H](c3cc(NC(=O)c4cnn(CCOC(F)(F)F)c4)n(C(C)(C)C)n3)C2)CC1]

Step 3. To a mixture of 1-(2-(trifluoromethoxy)ethyl)-1H-pyrazole-4-carboxylic acid (91 mg, 0.41 mmol) in EA (5 mL) was added (3R,5R)-5-(5-amino-1-(tert-butyl)-1H-pyrazol-3-yl)tetrahydrofuran-3-yl (1-methylcyclopropyl)carbamate (110 mg, 0.34 mmol) and DIEA (441 mg, 3.41 mmol), then T₃P (2.6 g, 50 wt, 2.04 mmol in EA) dropwise at 0° C. under nitrogen atmosphere. The mixture was stirred for 2.5 h at 80° C. The reaction mixture was diluted with H₂O (15 mL), and the aqueous phase was extracted with EA (30 mL) three times. The combined organic layers were washed with brine, dried over sodium sulfate, filtered. Concentration in vacuo resulted in (3R,5R)-5-(1-(tert-butyl)-5-(1-(2-(trifluoromethoxy)ethyl)-1H-pyrazole-4-carboxamido)-1H-pyrazol-3-yl)tetrahydrofuran-3-yl (1-methylcyclopropyl)carbamate (150 mg, 83.2%) as a white solid. m/z (ES⁺) [M+H]⁺=529.15; HPLC tR=0.783 min.

### (3R,5R)-5-(3-(1-(2-(trifluoromethoxy)ethyl)-1H-pyrazole-4-carboxamido)-1H-pyrazol-5-yl)tetrahydrofuran-3-yl (1-methylcyclopropyl)carbamate

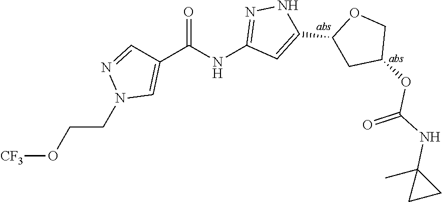 [SMILES: CC1(NC(=O)O[C@H]2CO[C@@H](c3cc(NC(=O)c4cnn(CCOC(F)(F)F)c4)n[nH]3)C2)CC1]

Step 4. A resealable reaction vial was charged with (3R,5R)-5-(1-(tert-butyl)-5-(1-(2-(trifluoromethoxy)ethyl)-1H-pyrazole-4-carboxamido)-1H-pyrazol-3-yl)tetrahydrofuran-3-yl (1-methylcyclopropyl)carbamate (145 mg, 0.27 mmol), FA (6 mL) and a stirbar before being evacuated and purged with nitrogen three times, and the mixture was stirred for 1.5 hour at 75° C. The reaction concentrated in vacuo. The resulting crude material was purified by Pre-HPLC (Column: XBridge Shield RP18 OBD Column, 30*150 mm, 5 μm; Mobile Phase A: Water (10 mmol/L NH₄HCO_{3+0.1}% NH₃.H₂O), Mobile Phase B: ACN; Flow rate: 60 mL/min; Gradient: 20% B to 40% B in 8 min, 40% B; Wave Length: 254 nm; RT1(min): 7.57). Lyophilization yielded (3R,5R)-5-(3-(1-(2-(trifluoromethoxy)ethyl)-1H-pyrazole-4-carboxamido)-1H-pyrazol-5-yl)tetrahydrofuran-3-yl (1-methylcyclopropyl)carbamate (72.1 mg, 55.6%) as a white amorphous solid. m/z (ES⁺) [M+H]⁺=473.35; HPLC tR=0.687 min. ¹H NMR (400 MHz, DMSO-d6) δ 12.39 (s, 1H), 10.49 (s, 1H), 8.41 (s, 1H), 8.13 (s, 1H), 7.53 (s, 1H), 6.54 (s, 1H), 5.16 (s, 1H), 4.81 (s, 1H), 4.51-4.44 (m, 4H), 3.84 (s, 2H), 2.75 (s, 1H), 1.91 (s, 1H), 1.25 (s, 3H), 0.61 (s, 2H), 0.48 (s, 2H).

Additional compounds prepared according to the methods of Example 45 are depicted in Table 16 below.

TABLE 16
Additional Exemplary Compounds

| | | | MS |
|---|---|---|---|
| Compound | Structure | ¹H NMR | [M + 1] |
| ((3S,5S)-5-(3- (1-(2- (trifluoro- methoxy) ethyl)- 1H-pyrazole- 4- carboxamido)- 1H-pyrazol- 5- yl)tetrahydro- furan-3-yl (1- methylcyclo- propyl) carbamate | 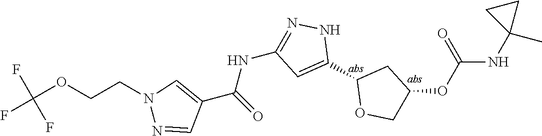 [SMILES: CC1(NC(=O)O[C@@H]2CO[C@H](c3cc(NC(=O)c4cnn(CCOC(F)(F)F)c4)n[nH]3)C2)CC1] | (400 MHz, DMSO-d6) 12.39 (s, 1H), 10.49 (s, 1H), 8.41 (s, 1H), 8.14 (s, 1H), 7.53 (s, 1H), 6.54 (s, 1H), 5.16 (s, 1H), 4.83 (t, J = 7.6 Hz, 1H), 4.54-4.43 (m, 4H), 3.84 (s, 2H), 2.70 (dt, J = 14.2, 7.3 Hz, 1H), 1.91 (s, 1H), 1.25 (s, 3H), 0.61 (s, 2H), 0.48 (q, J = 4.5 Hz, 2H). | 473.20 |

### Example 46

### (3R,5R)-5-(3-((2-((trifluoromethoxy)methyl) pyrazolo[1,5-a]pyrazin-4-yl)amino)-1H-pyrazol-5-yl)tetrahydrofuran-3-yl (1-methylcyclopropyl)carbamate

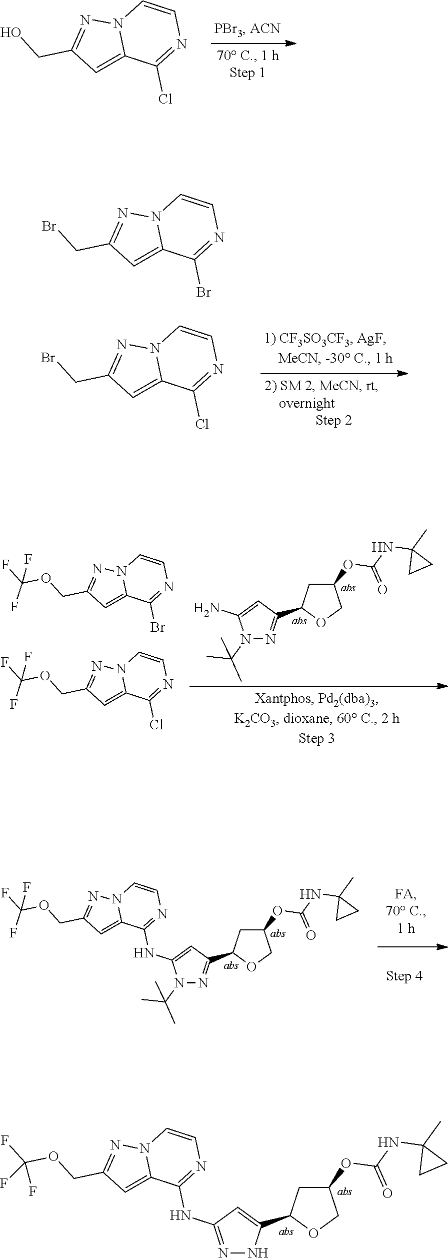

### 4-bromo-2-(bromomethyl)pyrazolo[1,5-a]pyrazine

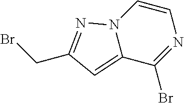 [SMILES: BrCc1cc2c(Br)nccn2n1]

Step 1. To a stirred solution of (4-chloropyrazolo[1,5-a]pyrazin-2-yl)methanol (2 g, 1 Eq, 0.01 mol) in MeCN (20 mL) was added PBr3 (4 g, 2 mL, 1.5Eq, 0.02 mol) at 0° C. under nitrogen atmosphere. The resulting mixture was stirred for 1 hour at 70° C. The mixture was adjusted to PH=7 and extracted with EtOAc (3×40 mL). The combined organic layers were washed with brine (1×40 mL), dried over anhydrous Na2SO4. After filtration, the filtrate was concentrated under reduced pressure to afford 4-bromo-2-(bromomethyl)pyrazolo[1,5-a]pyrazine (1.5 g, 5.2 mmol, 50%) as yellow solid. m/z (ES⁺) [M+H]⁺=290.80; HPLC tR=0.850 min.

### 4-bromo-2-((trifluoromethoxy)methyl)pyrazolo[1,5-a]pyrazine

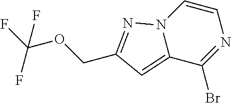 [SMILES: FC(F)(F)OCc1cc2c(Br)nccn2n1]

Step 2. To a stirred solution of Silver fluoride (2.0 g, 0.34 mL, 3 Eq, 15 mmol) in MeCN (30 mL) was added Trifluoromethyltriflate (4.5 g, 4 Eq, 21 mmol) at −30 C under nitrogen atmosphere. The mixture was stirred for 1 h at −30 C. Then 4-bromo-2-(bromomethyl)pyrazolo[1,5-a]pyrazine (1.5 g, 1 Eq, 5.2 mmol) was added. The resulting mixture was stirred for 16 hour at 25° C. The resulting mixture was filtered. The residue was purified by silica gel chromatography with the following conditions. EtOAc in PE, 0% to 25% gradient in 20 min to afford 4-bromo-2-((trifluoromethoxy)methyl)pyrazolo[1,5-a]pyrazine (600 mg, 2.03 mmol, 39%) as yellow oil. m/z (ES⁺) [M+H]⁺=295.80; HPLC tR=0.848 min.

### (3R,5R)-5-(1-(tert-butyl)-5-((2-((trifluoromethoxy)methyl)pyrazolo[1,5-a]pyrazin-4-yl)amino)-1H-pyrazol-3-yl)tetrahydrofuran-3-yl (1-methylcyclopropyl)carbamate

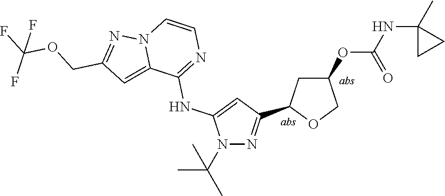 [SMILES: CC1(NC(=O)O[C@H]2CO[C@@H](c3cc(Nc4nccn5nc(COC(F)(F)F)cc45)n(C(C)(C)C)n3)C2)CC1]

Step 3. To a stirred solution of (3R,5R)-5-(5-amino-1-(tert-butyl)-1H-pyrazol-3-yl)tetrahydrofuran-3-yl (1-methylcyclopropyl)carbamate (327 mg, 1Eq, 1.01 mmol) in 1,4-Dioxane (3 mL) was added 4-bromo-2-((trifluoromethoxy)methyl)pyrazolo[1,5-a]pyrazine (300 mg, 1 Eq, 1.01 mmol), K₂CO₃ (420 mg, 3 Eq, 3.04 mmol), xantphos (235 mg, 0.4 Eq, 405 μmol), Pd₂(dba)₃ (186 mg, 0.2 Eq, 203 μmol) at room temperature under nitrogen atmosphere. The resulting mixture was stirred for 1 hour at 60° C. The residue was purified by reverse flash chromatography with the following conditions; column, C18 silica gel; mobile phase, MeCN in water, 10% to 90% gradient in 15 min: detector, UV 254 nm. Concentration in vacuo resulted in (3R,5R)-5-(1-(tert-butyl)-5-((2-((trifluoromethoxy)methyl)pyrazolo[1,5-a]pyrazin-4-yl)amino)-1H-pyrazol-3-yl)tetrahydrofuran-3-yl (1-methylcyclopropyl)carbamate (160 mg, 298 μmol, 29.4%) as yellow oil. m/z (ES⁺) [M+H]⁺=538.15; HPLC tR=0.888 min.

### (3R,5R)-5-(3-((2-((trifluoromethoxy)methyl) pyrazolo[1,5-a]pyrazin-4-yl)amino)-1H-pyrazol-5-yl)tetrahydrofuran-3-yl (1-methylcyclopropyl)carbamate

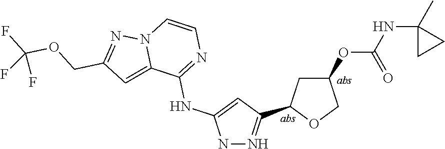 [SMILES: CC1(NC(=O)O[C@H]2CO[C@@H](c3cc(Nc4nccn5nc(COC(F)(F)F)cc45)[nH]n3)C2)CC1]

Step 4. (3R,5R)-5-(1-(tert-butyl)-5-((2-((trifluoromethoxy)methyl)pyrazolo[1,5-a]pyrazin-4-yl)amino)-1H-pyrazol-3-yl)tetrahydrofuran-3-yl (O-methylcyclopropyl)carbamate (150 mg, 1 Eq, 279 μmol) was added FA (3 mL). The reaction was stirred at 70° C. for 1 hour. The mixture was concentrated and purified by Prep-HPLC(Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 18% B to 40% B in 8 min, 40% B; Wave Length: 254 nm; RT1(min): 6.65). Lyophilization yielded (3R,5R)-5-(3-((2-((trifluoromethoxy)methyl) pyrazolo[1,5-a]pyrazin-4-yl)amino)-1H-pyrazol-5-yl)tetrahydrofuran-3-yl (1-methylcyclopropyl)carbamate (12.5 mg, 26.0 μmol, 9.30%) as a white solid. m/z (ES⁺) [M+H]⁺=482.20; HPLC tR=0.696 min. ¹H NMR (400 MHz, DMSO-d6) 12.39 (s, 1H), 10.16 (s, 1H), 8.10 (s, 1H), 7.58-7.21 (m, 3H), 6.80 (s, 1H), 5.35 (s, 2H), 5.17 (s, 1H), 4.92 (d, J=49.7 Hz, 1H), 3.86 (s, 2H), 2.73 (dd, J=14.7, 7.2 Hz, 1H), 1.96 (d, J=11.2 Hz, 1H), 1.25 (s, 3H), 0.60 (d, J=5.3 Hz, 2H), 0.48 (q, J=4.5 Hz, 2H).

Additional compounds prepared according to the methods of Example 46 are depicted in Table 16 below.

TABLE 16
Additional Exemplary Compounds

| | | | MS |
|---|---|---|---|
| Compound | Structure | ¹H NMR | [M + 1] |
| (3S,5S)-5-(3- ((2- ((trifluoro- methoxy)methyl) pyrazolo[1,5- a]pyrazin-4- yl)amino)-1H- pyrazol-5- yl)tetrahydro- furan-3-yl (1- methylcyclo- propyl) carbamate | 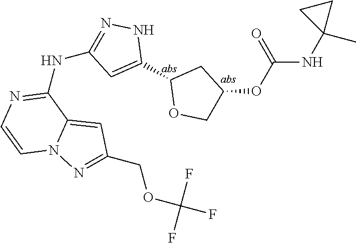 [SMILES: CC1(NC(=O)O[C@@H]2CO[C@H](c3cc(Nc4nccn5nc(COC(F)(F)F)cc45)n[nH]3)C2)CC1] | (400 MHz, DMSO-d6) 12.40 (s, 1H), 10.21 (s, 1H), 8.11 (d, J = 4.7 Hz, 1H), 7.59-7.39 (m, 3H), 6.77 (s, 1H), 5.35 (s, 2H), 5.17 (s, 1H), 4.84 (s, 1H), 3.85 (d, J = 6.3 Hz, 2H), 2.72 (dd, J = 14.3, 7.7 Hz, 1H), 1.97 (d, J = 12.7 Hz, 1H), 1.25 (s, 3H), 0.60 (d, J = 5.2 Hz, 2H), 0.48 (q, J = 5.0, 4.5 Hz, 2H). | 482.20 |

### Example 47

### (3S,5S)-5-(2-(3-(methoxymethyl)-1-methyl-1H-pyrazole-5-carboxamido)thiazol-5-yl)tetrahydrofuran-3-yl (1-methylcyclopropyl)carbamate

### (3R,5R)-5-(2-(3-(methoxymethyl)-1-methyl-1H-pyrazole-5-carboxamido)thiazol-5-yl)tetrahydrofuran-3-yl (1-methylcyclopropyl)carbamate

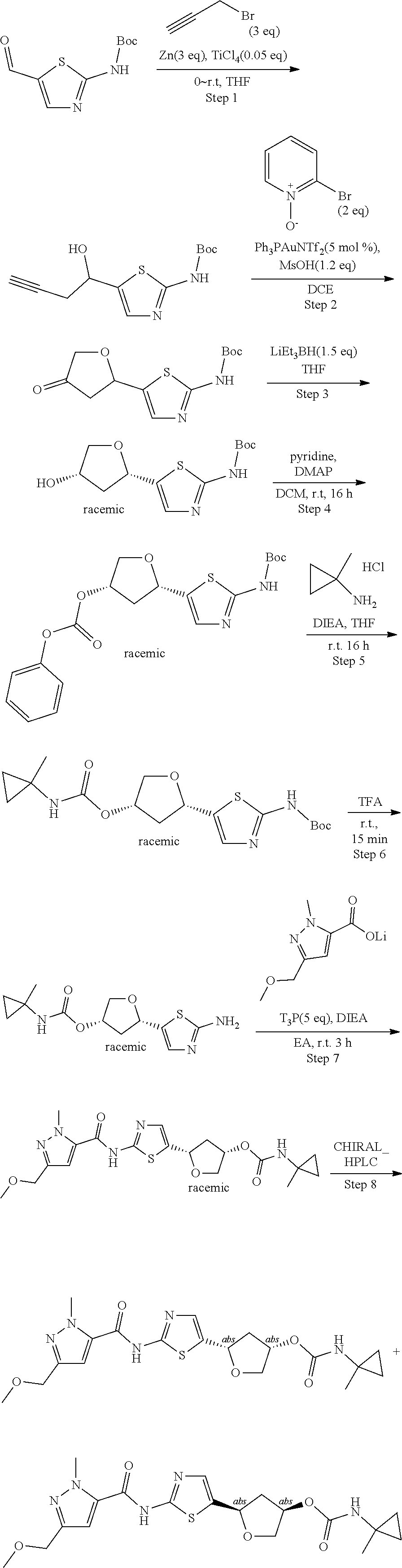

### tert-butyl (5-(1-hydroxybut-3-yn-1-yl)thiazol-2-yl)carbamate

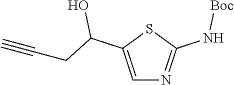 [SMILES: C#CCC(O)c1cnc(NC(=O)OC(C)(C)C)s1]

Step 1. A dry tricol flask equipped with un exit was charged with activated Zinc (1.75 g, 26.3 mmol) and dry THF (60.0 mL) was added under Argon. A solution of 80% propargyl bromide in toluene (2.83 mL, 26.3 mmol) was added at rt. The mixture was then cooled to 0-5° C. and a 1 M solution of Titanium(IV) chloride in DCM (438 uL, 438 umol) was slowly added. (Caution: the mixture became warm and vapors were released.) Addition was made slowly to permit vapor evacuate the flask. Once addition was finished, the ice bath was removed and the mixture was stirred for 20 minutes. A solution of tert-Butyl (5-formylthiazol-2-yl)carbamate (2.00 g, 8.76 mmol) in THF (24.0 mL) was then added dropwise over 20 minutes at rt and the mixture was stirred for 2 h. The mixture was poured into ice/saturated solution of NH₄Cl (30 mL) and EtOAc (70 mL) was added and the mixture was stirred for 5 minutes. The excess of zinc was then removed by filtration over celite and rinsed with EtOAC (30 mL). Organic phase was washed with a saturated solution of NaHCO₃(20 mL), brine (10 mL, 2X), dried over Na2SO4, filtered and concentrated under reduced pressure. Hexane (20 mL) was added and the mixture was concentrated under reduced pressure. This process was repeated 3 times. Ether (20 mL) was then added and the mixture was sonicated and then let stand in the fridge for 3-4 hours. The yellow liquid phase was then removed and the mixture was concentrated under reduced pressure and then dried over pump to afford the title compound as a yellow-pale solid (1.8 g, 77%). ESI-MS (m/z+): 213.1 [M+H]. Rf (50% EtOAc/hexane):0.23. ¹H NMR (400 MHz, CDCl3): S 10.48 (br s; 1H); 7.29 (s; 1 H); 5.07 (q; J=5.38 Hz; 1 H); 2.76 (dd; J=6.13; 2.62 Hz; 2 H); 2.45 (d; J=4.56 Hz; 1 H); 2.14 (t; J=2.58 Hz; 1 H); 1.57 (s; 9 H).

### tert-butyl (5-(4-oxotetrahydrofuran-2-yl)thiazol-2-yl)carbamate

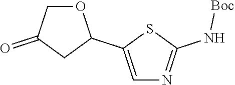 [SMILES: CC(C)(C)OC(=O)Nc1ncc(C2CC(=O)CO2)s1]

Step 2. A dry vial was charged with tert-butyl (5-(1-hydroxybut-3-yn-1-yl)thiazol-2-yl)carbamate (1.50 g, 5.59 mmol) and DCE (121 mL) was added under Argon. 2-bromopyridine 1-oxide (1.95 g, 11.2 mmol) was then added. The mixture became more soluble. Methanesulfonic acid (33.5 mL, 6.71 mmol) was added in one shot. A solution was obtained. Triphenylphosphinegold(I) bis(trifluoromethanesulfonyl)imidate (211 mg, 280 umol) was then added in one shot under argon and the solution was stirred at room temperature for 3 h. The mixture was then concentrated under reduced pressure. EtOAc (70 mL) and a saturated solution of NaHCO₃(30 mL) were added. Phases were separated and aqueous phase was extracted with EtOAc (30 mL, 2X). The combined organic phases were washed with water (20 mL), brine (10 mL) then dried over Na₂SO₄, filtered and concentrated under reduced pressure. The mixture was purified over a 25 g silicagel column using a mixture of EtOAc in hexane gradient (0-60%). The purified compound contained some impurities so it was triturated with ether. Solids were collected and put over pump to afford the title compound as a pale-yellow solid (760 mg, 48%). ESI-MS (m/z+): 229.0 [M+H-tBu]. Rf: 0.32 (50% EtOAc in hexane). ¹H NMR (400 MHz, CDCl3): S 10.91 (s; 1 H); 7.29 (s; 1 H); 5.51 (t; J=7.38 Hz; 1 H); 3.98-4.18 (m; 2 H); 2.87-2.93 (m; 1 H); 2.66 (dd; J=17.90; 8.06 Hz; 1 H); 1.58 (s; 9 H).

### racemic tert-butyl (5-((2S,4S)-4-hydroxytetrahydrofuran-2-yl)thiazol-2-yl)carbamate

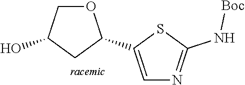 [SMILES: CC(C)(C)OC(=O)Nc1ncc([C@@H]2C[C@H](O)CO2)s1]

Step 3. To a solution of tert-butyl (5-(4-oxotetrahydrofuran-2-yl)thiazol-2-yl)carbamate (700 mg, 2.46 mmol) in dry THF (10.0 mL) was added Super-Hydride solution 1M in THF(3.69 mL, 3.69 mmol) dropwise at −65° C. under argon and the mixture was stirred for 1 h at −65° C. to −70° C. The mixture was then poured into ice/water/saturated solution of NH₄Cl (20 mL) and extracted with EtOAc (100 mL). Organic phase was then washed with a saturated solution of NaHCO₃(20 mL) and then with brine (10 mL), dried over Na₂SO₄, filtered and concentrated under reduced pressure. The crude compound was purified over a silicagel column using a mixture of EtOAC in hexane (0-100%) to afford the title compound as a white solid (550 mg, 78%). ESI-MS (m/z+): 231.1 (major peak [M+H-tBu]), 287.1 [M+H]. Rf: 0.65 (10% MeOH in DCM). ¹H NMR (400 MHz, CDCl3): δ 11.20 (br s; 1 H); 7.26 (s; 1 H); 5.11 (t; J=7.16 Hz; 1 H); 4.57 (br s; 1 H); 3.97-3.99 (m; 1 H); 3.87 (dd; J=9.88; 4.36 Hz; 1 H); 2.61-2.68 (m; 1 H); 2.07 (dd; J=13.97; 5.66 Hz; 1 H); 1.80-1.82 (m; 1H); 1.57 (s; 9 H).

### racemic (tert-butyl (5-((2S,4S)-4-(((4-nitrophenoxy)carbonyl)oxy)tetrahydrofuran-2-yl)thiazol-2-yl)carbamate

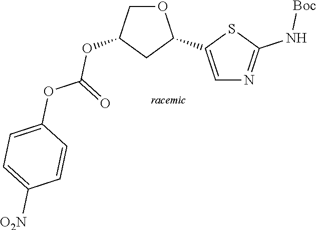 [SMILES: CC(C)(C)OC(=O)Nc1ncc([C@@H]2C[C@H](OC(=O)Oc3ccc([N+](=O)[O-])cc3)CO2)s1]

Step 4. To a stirred solution of racemic tert-butyl (5-((2S,4S)-4-hydroxytetrahydrofuran-2-yl)thiazol-2-yl)carbamate (500 mg, 1.75 mmol) in DCM (6 mL) was added pyridine (414 mg, 5.24 mmol) and DMAP (42.7 mg, 349 μmol) under 0° C. To above reaction was added 4-nitrophenyl carbonochloridate (528 mg, 2.62 mmol) under N₂. The reaction was stirred at 25° C. for 16 hour. The resulting mixture was concentrated under vacuum. The residue was purified by reverse flash chromatography with the following conditions: column, C18 silica gel; mobile phase, MeCN in water, 40% to 70% gradient in 15 min; detector, UV 254 nm. concentrated in vacuo resulted in racemic (tert-butyl (5-((2S,4S)-4-(((4-nitrophenoxy)carbonyl)oxy)tetrahydrofuran-2-yl)thiazol-2-yl)carbamate (750 mg, 1.5 mmol, 88%, 92% Purity)) as white oil. m/z (ES⁺) [M+H]⁺=452.25; HPLC tR=0.915 min.

### racemic tert-butyl (5-((2S,4S)-4-(((1-methylcyclopropyl)carbamoyl)oxy)tetrahydrofuran-2-yl)thiazol-2-yl)carbamate

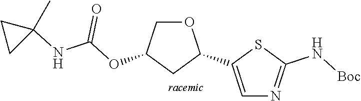 [SMILES: CC1(NC(=O)O[C@@H]2CO[C@H](c3cnc(NC(=O)OC(C)(C)C)s3)C2)CC1]

Step 5. To a round bottomed flask was charged with racemic tert-butyl (5-((2S,4S)-4-(((4-nitrophenoxy)carbonyl)oxy)tetrahydrofuran-2-yl)thiazol-2-yl)carbamate (750 mg, 1.66 mmol), DIEA (429 mg, 3.32 mmol), 1-methylcyclopropan-1-amine hydrochloride (715 mg, 6.65 mmol), THF (10 mL) and a stirbar. The solution was stirred for 16 h at 25° C. under nitrogen atmosphere. LCMS showed the reaction was completed. The resulting mixture was concentrated under vacuum. The residue was purified by reverse flash chromatography with the following conditions: column, C18 silica gel; mobile phase, MeCN in water, 30% to 60% gradient in 15 min; detector, UV 254 nm. Concentrated in vacuo resulted in racemic tert-butyl (5-((2S,4S)-4-(((1-methylcyclopropyl)carbamoyl)oxy)tetrahydrofuran-2-yl)thiazol-2-yl)carbamate (450 mg, 1.1 mmol, 65%, 92% Purity) as a light yellow oil. m/z (ES⁺) [M+H]⁺=384.35; HPLC tR=0.790 min.

### racemic (3S,5S)-5-(2-aminothiazol-5-yl)tetrahydrofuran-3-yl (1-methylcyclopropyl)carbamate

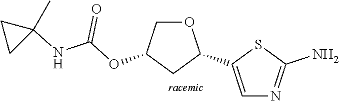 [SMILES: CC1(NC(=O)O[C@@H]2CO[C@H](c3cnc(N)s3)C2)CC1]

Step 6. A round bottomed flask was charged with racemic tert-butyl (5-((2S,4S)-4-(((1-methylcyclopropyl)carbamoyl)oxy)tetrahydrofuran-2-yl)thiazol-2-yl)carbamate (350 mg, 913 μmol), and a stirbar. TFA (3 mL) was added, and the solution was stirred for 15 min at 25° C. The mixture was neutralized to pH 7 with sat. NaHCO₃ solution. The reaction mixture was diluted with water (10 mL), and the aqueous phase was extracted with EA (50 mL) three times. The combined organic layers were washed with brine, dried over sodium sulfate, filtered, and concentrated in vacuo to afford racemic (3S,5S)-5-(2-aminothiazol-5-yl)tetrahydrofuran-3-yl (1-methylcyclopropyl)carbamate (250 mg, 882 μmol, 96.7%) as a white solid. m/z (ES⁺) [M+H]=284.20; HPLC tR=0.464 min.

### racemic (3S,5S)-5-(2-(3-(methoxymethyl)-1-methyl-1H-pyrazole-5-carboxamido)thiazol-5-yl)tetrahydrofuran-3-yl (1-methylcyclopropyl)carbamate

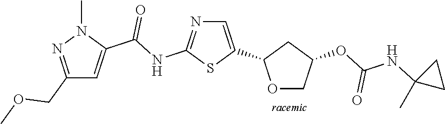 [SMILES: COCc1cc(C(=O)Nc2ncc([C@@H]3C[C@H](OC(=O)NC4(C)CC4)CO3)s2)n(C)n1]

Step 7. To a mixture of lithium 3-(methoxymethyl)-1-methyl-1H-pyrazole-5-carboxylate (56 mg, 0.32 mmol), racemic (3S,5S)-5-(2-aminothiazol-5-yl)tetrahydrofuran-3-yl (1-methylcyclopropyl)carbamate (75 mg, 0.26 mmol) and DIEA (0.44 g, 3.4 mmol) in EA (3 mL) was added 2,4,6-tripropyl-1,3,5,2,4,6-trioxatriphosphinane 2,4,6-trioxide (0.84 g, 50% Wt, 1.3 mmol) dropwise at 0° C. under nitrogen atmosphere. The mixture was stirred for 3 h at 25° C. The reaction mixture was diluted with water (7 mL), and the aqueous phase was extracted with EA (20 mL) three times. The combined organic layers were washed with brine, dried over sodium sulfate, filtered, and concentrated in vacuo. The residue was purified by Prep-TLC (EA/DCM; ratio:15/1) to afford racemic (3S,5S)-5-(2-(3-(methoxymethyl)-1-methyl-1H-pyrazole-5-carboxamido)thiazol-5-yl)tetrahydrofuran-3-yl (1-methylcyclopropyl)carbamate (50 mg, 0.11 mmol, 43%) as a white solid. m/z (ES⁺) [M+H]⁺=436.30; HPLC tR=0.710 min.

### (3S,5S)-5-(2-(3-(methoxymethyl)-1-methyl-1H-pyrazole-5-carboxamido)thiazol-5-yl)tetrahydrofuran-3-yl (1-methylcyclopropyl)carbamate

### (3R,5R)-5-(2-(3-(methoxymethyl)-1-methyl-1H-pyrazole-5-carboxamido)thiazol-5-yl)tetrahydrofuran-3-yl (1-methylcyclopropyl)carbamate

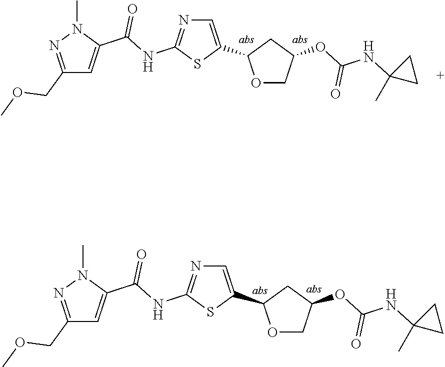 [SMILES: COCc1cc(C(=O)Nc2ncc([C@@H]3C[C@H](OC(=O)NC4(C)CC4)CO3)s2)n(C)n1 ; COCc1cc(C(=O)Nc2ncc([C@H]3C[C@@H](OC(=O)NC4(C)CC4)CO3)s2)n(C)n1]

Step 8. racemic (3S,5S)-5-(2-(3-(methoxymethyl)-1-methyl-1H-pyrazole-5-carboxamido)thiazol-5-yl)tetrahydrofuran-3-yl (1-methylcyclopropyl)carbamate (50 mg, 1 Eq, 0.11 mmol) was separated by chiral separation (HPLC) with the following conditions(Column: DZ-CHIRALPAK IG-3, 4.6*50 mm, 3.0 μm; Mobile Phase A: Hex(0.2% DEA): (EtOH: DCM=1:1)=60:40; Flow rate: 1 mL/min; Gradient: 0% B to 0% B; Injection Volume: Sul mL) to afford (3S,5S)-5-(2-(3-(methoxymethyl)-1-methyl-1H-pyrazole-5-carboxamido)thiazol-5-yl)tetrahydrofuran-3-yl (1-methylcyclopropyl)carbamate (9.5 mg, 20 μmol, 34%, 90.3% Purity) as a white solid. m/z (ES⁺) [M+H]⁺=436.25; HPLC tR=1.255 min. ¹H NMR (400 MHz, DMSO-d6) 12.62 (s, 1H), 7.56 (s, 2H), 7.28 (s, 1H), 5.17 (s, 1H), 5.04 (t, J=7.4 Hz, 1H), 4.37 (s, 2H), 4.11 (s, 3H), 3.85 (m, 2H), 3.28 (s, 3H), 2.76 (dt, J=14.4, 7.5 Hz, 1H), 1.88 (d, J=11.3 Hz, 1H), 1.25 (d, J=6.3 Hz, 3H), 0.63 (s, 2H), 0.49 (s, 2H).

Racemic (3S,5S)-5-(2-(3-(methoxymethyl)-1-methyl-1H-pyrazole-5-carboxamido)thiazol-5-yl)tetrahydrofuran-3-yl (1-methylcyclopropyl)carbamate (50 mg, 0.11 mmol) was separated by chiral separation (HPLC) with the following conditions(Column: DZ-CHIRALPAK IG-3, 4.6*50 mm, 3.0 μm; Mobile Phase A: Hex (0.2% DEA): (EtOH: DCM=1:1)=60:40; Flow rate: 1 mL/min; Gradient: 0% B to 0% B; Injection Volume: Sul mL) to afford (3R,5R)-5-(2-(3-(methoxymethyl)-1-methyl-1H-pyrazole-5-carboxamido)thiazol-5-yl)tetrahydrofuran-3-yl (1-methylcyclopropyl)carbamate (9.3 mg, 21 μmol, 18%, 97.7% Purity) as a white solid.m/z (ES⁺) [M+H]⁺=436.20; HPLC tR=1.250 min ¹H NMR (400 MHz, DMSO-d6) 12.62 (s, 1H), 7.56 (s, 2H), 7.28 (s, 1H), 5.17 (s, 1H), 5.04 (t, J=7.6 Hz, 1H), 4.36 (s, 2H), 4.10 (s, 3H), 3.85 (m, 2H), 3.27 (s, 3H), 2.75 (dt, J=14.4, 7.5 Hz, 1H), 1.89 (t, J=11.0 Hz, 1H), 1.25 (d, J=6.9 Hz, 3H), 0.64 (s, 2H), 0.50 (q, J=4.1 Hz, 2H).

### Example 48

### (3R,5R)-5-(3-((2-(methoxymethyl)imidazo[1,2-c]pyrimidin-5-yl)amino)-1H-pyrazol-5-yl)tetrahydrofuran-3-yl (1-methylcyclopropyl)carbamate

### (3R,5S)-5-(3-((2-(methoxymethyl)imidazo[1,2-c]pyrimidin-5-yl)amino)-1H-pyrazol-5-yl)tetrahydrofuran-3-yl (1-methylcyclopropyl)carbamate

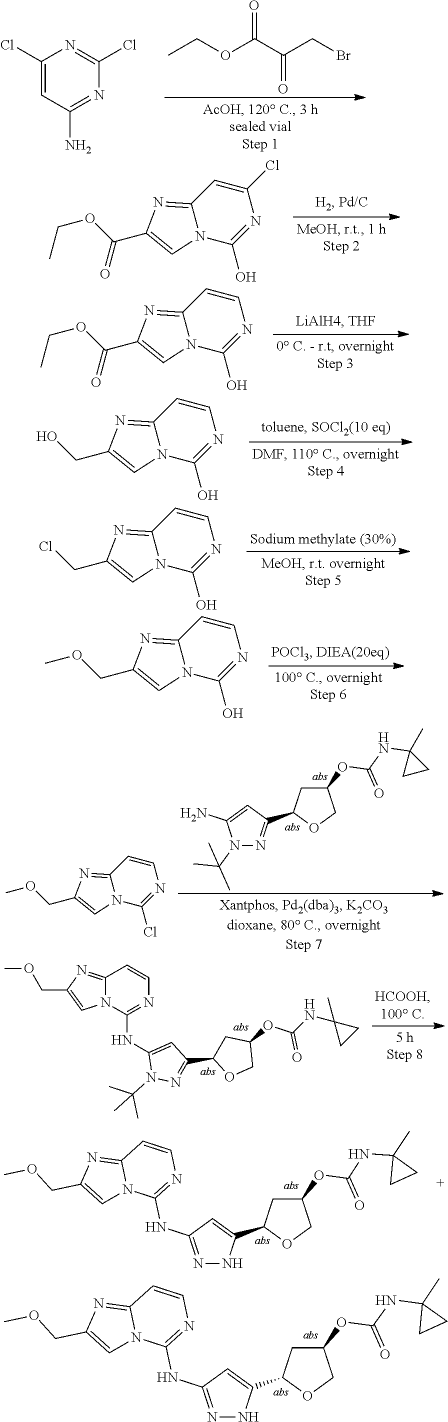

### ethyl 7-chloro-5-hydroxyimidazo[1,2-c]pyrimidine-2-carboxylate

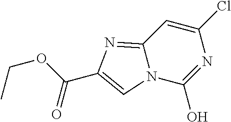 [SMILES: CCOC(=O)c1cn2c(O)nc(Cl)cc2n1]

Step 1. A round bottomed flask was charged with 2,6-dichloropyrimidin-4-amine (3 g, 0.02 mol), ethyl 3-bromo-2-oxopropanoate (9 g, 0.05 mol) and a stirbar. AcOH (32 mL) was added, and the solution was stirred for 3 hours at 120° C. The mixture was concentrated in vacuum. The mixture was adjusted pH value to 6-7. The aqueous phase was extracted with DCM (50 mL) three times. The combined organic layers were washed with brine, dried over sodium sulfate, filtered, and concentrated in vacuum. The residue was purified by silica gel chromatography (10 g column; eluting with DCM/MEOH; ratio:30/1). Concentration in vacuum. The precipitated solids were collected by filtration and washed with EA (20 mL) to afford ethyl 7-chloro-5-hydroxyimidazo[1,2-c]pyrimidine-2-carboxylate (1.09 g, 4.51 mmol, 20%) as a light pink solid. m/z (ES⁺) [M+H]⁺=242.05; HPLC tR=0.467 min.

### ethyl 5-hydroxyimidazo[1,2-c]pyrimidine-2-carboxylate

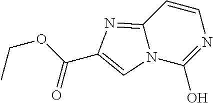

Step 2. A stirred mixture of ethyl 7-chloro-5-hydroxyimidazo[1,2-c]pyrimidine-2-carboxylate (1.09 g, 4.51 mmol) and Pd/C (120 mg, 1.13 mmol) in MeOH (15 mL) was treated with H₂ for 1 hour at 25° C. The reaction mixture was filtered through a pad of Celite, the pad was washed with MeOH (50 ml), and the filtrate was concentrated in vacuum to afford ethyl 5-hydroxyimidazo[1,2-c]pyrimidine-2-carboxylate (910 mg, 4.39 mmol, 97.4%) as a yellow solid. m/z (ES⁺) [M+H]⁺=208.05; HPLC tR=0.615 min.

### 2-(hydroxymethyl)imidazo[1,2-c]pyrimidin-5-ol

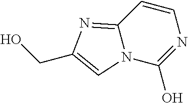 [SMILES: OCc1cn2c(O)nccc2n1]

Step 3. To a mixture of ethyl 5-hydroxyimidazo[1,2-c]pyrimidine-2-carboxylate (500 mg, 2.41 mmol) in THF (15 mL) was added LAH (3.62 mL of a solution 1M in THF, 3.62 mmol) dropwise at 0° C. under nitrogen atmosphere. The mixture was stirred for 12 hours at 25° C. The mixture was quenched with Na₂SO₄·10H₂O. The reaction mixture was filtered, the pad was washed with tepidity DCM/MeOH=4/1(100 ml) and MeOH/H₂O=4/1(100 ml), and the filtrate was concentrated in vacuum to afford 2-(hydroxymethyl)imidazo[1,2-c]pyrimidin-5-ol (480 mg, 2.91 mmol, crude) as a brown solid. m/z (ES⁺) [M+H]⁺=166.05; HPLC tR=0.233 min.

### 2-(chloromethyl)imidazo[1,2-c]pyrimidin-5-ol

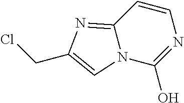 [SMILES: Oc1nccc2nc(CCl)cn12]

Step 4. A round bottomed flask was charged with 2-(hydroxymethyl)imidazo[1,2-c]pyrimidin-5-ol (2.7 g,16 mmol), SOCl₂ (19 g, 12 mL, 0.16 mol), DMF (0.01 mL), toluene (30 mL) and a stirbar. The solution was stirred for 5 hours at 110° C. The mixture was concentrated in vacuum to afford 2-(chloromethyl)imidazo[1,2-c]pyrimidin-5-ol (2.1 g, 11 mmol, crude) as a brown solid. m/z (ES⁺) [M+H]⁺=184.00; HPLC tR=0.565 min.

### 2-(methoxymethyl)imidazo[1,2-c]pyrimidin-5-ol

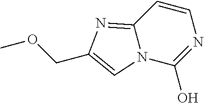

Step 5. A round bottomed flask was charged with 2-(chloromethyl)imidazo[1,2-c]pyrimidin-5-ol (2.1 g, 11 mmol), Sodium methoxide (10 g, 11 mL, 30% Wt, 57 mmol) and a stirbar. MeOH (40 mL) was added, and the solution was stirred for 16 hours at 25° C. The mixture was concentrated in vacuum. Adjusted pH value of the mixture to 6-7 with 2M HCl. The residue was purified by reverse flash chromatography with the following conditions: column, C18 silica gel; mobile phase, MeCN in water, 0% to 20% gradient in 25 min; detector, UV 254 nm to afford 2-(methoxymethyl)imidazo[1,2-c]pyrimidin-5-ol (380 mg, 2.12 mmol, 19%) as a white amorphous solid. m/z (ES⁺) [M+H]⁺=180.05; HPLC tR=0.432 min

### 5-chloro-2-(methoxymethyl)imidazo[1,2-c]pyrimidine

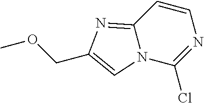

Step 6. A round bottomed flask was charged with 2-(methoxymethyl)imidazo[1,2-c]pyrimidin-5-ol (380 mg, 2.12 mmol), DIEA (5.48 g, 7.39 mL, 42.4 mmol), POCl₃ (25 mL) and a stirbar. The solution was stirred for 6 hours at 100° C. The mixture was concentrated in vacuum. Adjusted pH value of the mixture to 6-7 with NaHCO₃. The reaction mixture was diluted with water (20 mL), and the aqueous phase was extracted with EA (20 mL) three times. The combined organic layers were washed with brine, dried over sodium sulfate, filtered, and concentrated in vacuum. The residue was purified by reverse flash chromatography with the following conditions: column, C18 silica gel; mobile phase, MeCN in water, 0% to 50% gradient in 18 min; detector, UV 254 nm to afford 5-chloro-2-(methoxymethyl)imidazo[1,2-c]pyrimidine (320 mg, 1.62 mmol, 76.4%) as a white solid. m/z (ES⁺) [M+H]⁺=198.00; HPLC tR=0.656 min

### (3R,5R)-5-(1-(tert-butyl)-5-((2-(methoxymethyl)imidazo[1,2-c]pyrimidin-5-yl)amino)-1H-pyrazol-3-yl)tetrahydrofuran-3-yl (1-methylcyclopropyl)carbamate

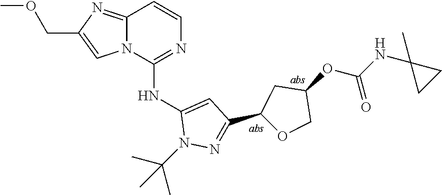 [SMILES: COCc1cn2c(Nc3cc([C@H]4C[C@@H](OC(=O)NC5(C)CC5)CO4)nn3C(C)(C)C)nccc2n1]

Step 7. A resealable reaction vial was charged with (3R,5R)-5-(5-amino-1-(tert-butyl)-1H-pyrazol-3-yl)tetrahydrofuran-3-yl (1-methylcyclopropyl)carbamate (475 mg, 1.47 mmol), 5-chloro-2-(methoxymethyl)imidazo[1,2-c]pyrimidine (320 mg, 1.62 mmol), xantphos (426 mg, 736 μmol), Pd₂(dba)₃ (270 mg, 294 μmol), K₂CO₃ (610 mg, 4.42 mmol) and a stirbar before being evacuated and purged with nitrogen three times. 1,4-Dioxane (8 mL) was added, and the mixture was stirred for 12 hours at 80° C. The mixture was quenched with water (20 mL), and the aqueous phase was extracted with EA (30 mL) three times. The combined organic layers were washed with brine, dried over sodium sulfate, filtered, and concentrated in vacuum. The residue was purified by reverse flash chromatography with the following conditions: column, C18 silica gel; mobile phase, MeCN in water, 0% to 80% gradient in 18 min; detector, UV 254 nm to afford (3R,5R)-5-(1-(tert-butyl)-5-((2-(methoxymethyl)imidazo[1,2-c]pyrimidin-5-yl)amino)-1H-pyrazol-3-yl)tetrahydrofuran-3-yl (1-methylcyclopropyl)carbamate (260 mg, 538 μmol, 36.5%) as a white solid. m/z (ES⁺) [M+H]⁺=484.30; HPLC tR=0.913 min.

### (3R,5R)-5-(3-((2-(methoxymethyl)imidazo[1,2-c]pyrimidin-5-yl)amino)-1H-pyrazol-5-yl)tetrahydrofuran-3-yl (1-methylcyclopropyl)carbamate

### (3R,5S)-5-(3-((2-(methoxymethyl)imidazo[1,2-c]pyrimidin-5-yl)amino)-1H-pyrazol-5-yl)tetrahydrofuran-3-yl (1-methylcyclopropyl)carbamate

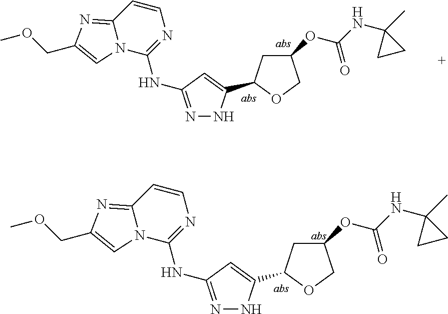

Step 8. A round bottomed flask was charged with (3R,5R)-5-(1-(tert-butyl)-5-((2-(methoxymethyl)imidazo[1,2-c]pyrimidin-5-yl)amino)-1H-pyrazol-3-yl)tetrahydrofuran-3-yl (1-methylcyclopropyl)carbamate (300 mg, 620 μmol) and a stirbar. HCOOH (8 mL) was added, and the solution was stirred for 5 hours at 100° C. The mixture was adjusted pH value to 6-7. The aqueous phase was extracted with EA (20 mL) three times. The combined organic layers were washed with brine, dried over sodium sulfate, filtered, and concentrated in vacuum. The resulting crude material was purified by Pre-HPLC (Column: XBridge Shield RP18 OBD Column, 30*150 mm, 5 μm; Mobile Phase A: Water(10 mmol/L NH₄HCO₃), Mobile Phase B: ACN; Flow rate: 60 mL/min; Gradient: 6% B to 32% B in 11 min, 32% B to 40% B in 12 min, 40% B; Wave Length: 220 nm; RT1(min): 10.47/11.2). Lyophilization yielded (3R,5R)-5-(3-((2-(methoxymethyl)imidazo[1,2-c]pyrimidin-5-yl)amino)-1H-pyrazol-5-yl)tetrahydrofuran-3-yl (1-methylcyclopropyl)carbamate (49.2 mg, 115 μmol, 18.6%) and (3R,5S)-5-(3-((2-(methoxymethyl)imidazo[1,2-c]pyrimidin-5-yl)amino)-1H-pyrazol-5-yl)tetrahydrofuran-3-yl (1-methylcyclopropyl)carbamate (57.4 mg, 134 μmol, 21.6%) as a white solid.

(3R,5R)-5-(3-((2-(methoxymethyl)imidazo[1,2-c]pyrimidin-5-yl)amino)-1H-pyrazol-5-yl)tetrahydrofuran-3-yl (1-methylcyclopropyl)carbamate: m/z (ES⁺) [M+H]⁺=428.20; HPLC tR=1.218 min. ¹H NMR (400 MHz, DMSO-d6) δ 12.51 (s, 1H), 10.12 (s, 1H), 8.31 (s, 1H), 7.59 (d, J=37.8 Hz, 2H), 7.08 (d, J=130.3 Hz, 1H), 6.67 (d, J=245.9 Hz, 1H), 5.17 (s, 1H), 4.86 (s, 1H), 4.50 (s, 2H), 3.85 (d, J=5.0 Hz, 2H), 3.35 (s, 3H), 2.69 (d, J=14.6 Hz, 1H), 2.02 (d, J=50.7 Hz, 1H), 1.24 (s, 3H), 0.60 (d, J=5.1 Hz, 2H), 0.52-0.36 (m, 2H).

(3R,5S)-5-(3-((2-(methoxymethyl)imidazo[1,2-c]pyrimidin-5-yl)amino)-1H-pyrazol-5-yl)tetrahydrofuran-3-yl (1-methylcyclopropyl)carbamate: m/z (ES⁺) [M+H]⁺=428.20; HPLC tR=1.306 min. ¹H NMR (400 MHz, DMSO-d6) δ 12.57 (s, 1H), 10.13 (s, 1H), 8.31 (s, 1H), 8.02-7.14 (m, 2H), 6.92 (d, J=6.4 Hz, 1H), 6.69 (d, J=251.5 Hz, 1H), 5.25 (s, 1H), 5.03 (s, 1H), 4.50 (s, 2H), 4.13 (d, J=15.7 Hz, 1H), 3.74 (d, J=10.3 Hz, 1H), 3.36 (s, 3H), 2.30 (d, J=24.5 Hz, 2H), 1.27 (s, 3H), 0.69-0.59 (m, 2H), 0.51 (d, J=5.3 Hz, 2H).

Additional compounds prepared according to the methods of Example 48 are depicted in Table 17 below.

TABLE 17
Additional Exemplary Compounds

| | | | MS |
|---|---|---|---|
| Compound | Structure | ¹H NMR | [M + 1] |
| (3S,5S)-5-(3- ((2- (methoxy- methyl) imidazo[1,2- c]pyrimidin- 5-yl)amino)- 1H-pyrazol-5- yl)tetrahydro- furan-3-yl (1- methylcyclo- propyl) carbamate | 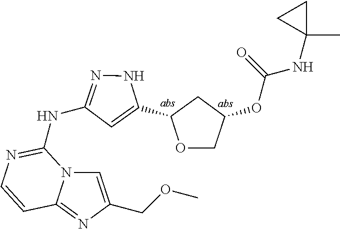 [SMILES: COCc1cn2c(Nc3cc([C@@H]4C[C@H](OC(=O)NC5(C)CC5)CO4)[nH]n3)nccc2n1] | (400 MHz, DMSO-d6) 12.51 (s, 1H), 10.13 (s, 1H), 8.31 (s, 1H), 7.59 (d, J = 39.8 Hz, 2H), 7.08 (d, J = 129.3 Hz, 1H), 6.67 (d, J = 248.4 Hz, 1H), 5.18 (s, 1H), 4.92 (d, J = 46.2 Hz, 1H), 4.50 (s, 2H), 3.86 (s, 2H), 3.35 (s, 3H), 2.70 (d, J = 15.3 Hz, 1H), 2.04 (d, J = 67.7 Hz, 1H), 1.24 (s, 3H), 0.60 (s, 2H), 0.48 (s, 2H). | 428.20 |
| (3S,5R)-5-(3- ((2- (methoxy- methyl) imidazo[1,2- c]pyrimidin- 5-yl)amino)- 1H-pyrazol-5- yl)tetrahydro- furan-3-yl (1- methylcyclo- propyl) carbamate | 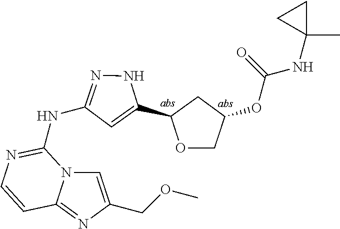 [SMILES: COCc1cn2c(Nc3cc([C@H]4C[C@H](OC(=O)NC5(C)CC5)CO4)[nH]n3)nccc2n1] | (400 MHz, DMSO-d6) 12.58 (s, 1H), 10.14 (s, 1H), 8.31 (s, 1H), 7.61 (s, 2H), 6.92 (s, 1H), 6.70 (s, 1H), 5.25 (s, 1H), 5.03 (s, 1H), 4.50 (s, 2H), 4.11 (d, J = 10.2 Hz, 1H), 3.74 (d, J = 10.3 Hz, 1H), 3.35 (s, 3H), 2.31 (d, J = 24.0 Hz, 2H), 1.27 (s, 3H), 0.74-0.58 (m, 2H), 0.51 (s, 2H). | 428.20 |

### Example 49

Compounds of the present disclosure were tested in a CDK2/Cyclin E fluorescence-based microfluidic mobility shift assay (PerkinElmer).

The active wild type CDK2/Cyclin E complex was purchased from Eurofins (14-475). Compound stocks were prepared in DMSO and were serially diluted into 11 concentrations by 3-fold dilution. Into a 384-well plate (Greiner, 781201) 200 nL compounds were transferred to individual wells prior to adding 15 uL of 1.3× enzyme and ATP solution containing 0.13 nM CDK2/CyclinE and 2660 uM ATP in 1× reaction buffer containing 10 mM Hepes pH 7.5, 0.01% Brij-35, 10 mM MgCl₂, 1 mM EGTA, 0.05% BSA and 2 mM DTT. Compounds were incubated in the presence of CDK2/Cyclin E complex and ATP for 30 minutes at room temperature. Catalytic reaction was initiated with the addition of 5 uL of a 4× peptide solution containing 6 uM fluorescently label substrate peptide, FL Peptide 18 (aa sequence 5-FAM-QSPKKG, PerkinElmer, 760362) in 1× reaction buffer.

The final reaction components were 0.1 nM CDK2/Cyclin E, 2000 uM ATP and 1.5 uM FL Peptide 18 and 1% DMSO. Reactions were incubated RT for 20 hr and terminated with 75 uL of a stopping solution containing 0.5 M EDTA. Samples were analyzed using a LabChip EZ reader (PerkinElmer).

Results of the CDK2 Biochemical Caliper Assay are presented in Table 1. Compounds having an IC₅₀ less than or equal to 100 nM are represented as “A”; compounds having an IC₅₀ greater than 100 nM but less than or equal to 250 nM are represented as “B”; compounds having an IC₅₀ greater than 250 nM but less than or equal to 1 μM are represented as “C”; and compounds having an IC₅₀ greater than 1 μM but less than or equal to 100 μM are represented as

### Example 50

Compounds described herein were also tested in a nanoBRET TE Intracellular Kinase Assay. Test compounds were prepared in a DMSO stock solution. 45 uL of stock solution was transferred to a 384 well plate (Greiner, 781201), and a 3-fold 11-point dilution was performed. 293-NB2 cells expressing target tagged with nanoLuciferase were diluted in cell growth media (DMEM+10% FBS+1% Pen/Strep media) and a 40 uL cell solution of 7.5E4 cells/mL was seeded in a separate 384 well cell culture plate and incubated overnight at at 37° C./5% CO_{2/100}% humidity. 2 uL of 20× Complete NanoBRET K-10 Tracer (Promega NanoBRET) was added to each well of the cell plate except control wells. 80 nL of each concentration of compound was transferred from the compound plate to a corresponding well in the cell culture plate by Echo550 liquid handler. The plates were incubated for 2 hrs at 37° C./5% CO_{2/100}% humidity. 20 uL of 3× Complete Nano-Glo Substrate & ECD NanoLuc Inhibitor (Promega NanoBRET) was added into each well. Chemiluminescence was then read on an EnVision reader (PerkinElmer).

Results of the Cell nanoBRET Assay are presented in Table 1. Compounds having an IC₅₀ less than or equal to 100 nM are represented as “A”; compounds having an IC₅₀ greater than 100 nM but less than or equal to 250 nM are represented as “B”; compounds having an IC₅₀ greater than 250 nM but less than or equal to 1 μM are represented as “C”; and compounds having an IC₅₀ greater than 1 μM but less than or equal to 100 μM are represented as “D”.

### Example 51

Compounds described herein were also tested in cell proliferation selectivity assays. OVCAR3, HCC1569, and SKOV3 cell lines were obtained from ATCC and cultured as recommended by supplier. COV318, COV504, and COV644 cell lines were obtained from Sigma-Aldrich and were cultured as recommended by supplier. The genetic background and dependency of each cell line was determined using publicly available databases: cBioPortal and DepMap. The cell lines that were amplified for CCNEJ (COV318, OVCAR3, and HCC1569) were found to be CDK2-dependent, whereas cell lines that were wildtype for CCNEJ (SKOV3, COV504, COV644) were found to be CDK2-independent. All six cell lines are CDK1-dependent given that CDK1 is an essential gene. Proliferation assay was carried out by seeding cells into 384-well plates at predetermined density (ranging from 150 to 1500 cells per well) in corresponding culture media, followed by brief centrifugation at 1000×g, and culturing cells for 24 hours at 37° C., 5% CO₂. The following day, compounds were serially diluted (1l-point, 3-fold dilution scheme with 20 μM as top concentration) and dispensed directly onto cells using Echo 555 acoustic liquid handler (Labcyte). The cells were incubated with the compounds at 37° C., 5% CO₂ for 120 hours. Relative number of viable cells in each well was determined using CyQUANT Direct Cell Proliferation Assay Kit (Invitrogen) per manufacturers protocol. The plates were read on EnVision 2105 multimode plate reader (Perkin Elmer) using FITC filter set. Dose response curve fitting and IC₅₀ calculation was done using Genedata Screener software. Proliferation selectivity (i.e. selectivity index) for individual compound was derived as a mean of its IC₅₀ ratios between each CDK2-independent versus CDK2-dependent cell line.

Proliferation selectivity index values for exemplary compounds are shown in Table 18 below. As appreciated by one of skill in the art, small increases in the magnitude of the proliferation selectivity index may indicate a substantially improved therapeutic index for treating CDK2-mediated disorders.

TABLE 18
Exemplary Compound Proliferation Selectivity Index Values

| | | Proliferation |
|---|---|---|
| | | selectivity |
| Example | Structure | Index |
| I-1 | 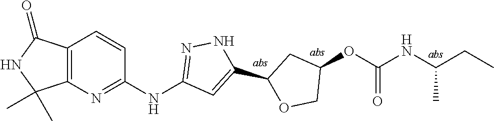 [SMILES: CC[C@H](C)NC(=O)O[C@H]1CO[C@@H](c2cc(Nc3ccc4c(n3)C(C)(C)NC4=O)n[nH]2)C1] | 54.25 |
| I-2 | 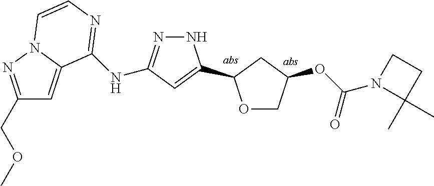 [SMILES: COCc1cc2c(Nc3cc([C@H]4C[C@@H](OC(=O)N5CCC5(C)C)CO4)[nH]n3)nccn2n1] | 51.61 |
| I-3 | 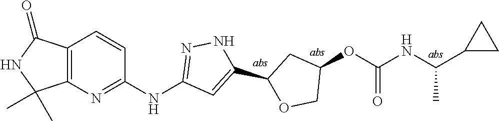 [SMILES: C[C@H](NC(=O)O[C@H]1CO[C@@H](c2cc(Nc3ccc4c(n3)C(C)(C)NC4=O)n[nH]2)C1)C1CC1] | 45.44 |
| I-4 | 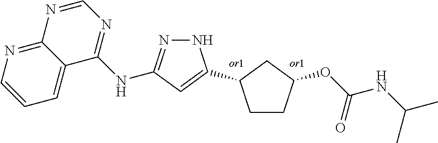 [SMILES: CC(C)NC(=O)O[C@@H]1CC[C@H](c2cc(Nc3ncnc4ncccc34)n[nH]2)C1] | 45.05 |
| I-5 | 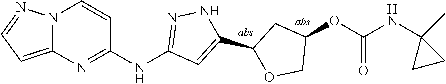 [SMILES: CC1(NC(=O)O[C@H]2CO[C@@H](c3cc(Nc4ccn5nccc5n4)n[nH]3)C2)CC1] | 32.74 |
| I-6 | 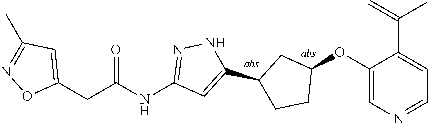 [SMILES: C=C(C)c1ccncc1O[C@H]1CC[C@@H](c2cc(NC(=O)Cc3cc(C)no3)n[nH]2)C1] | 31.48 |
| I-7 | 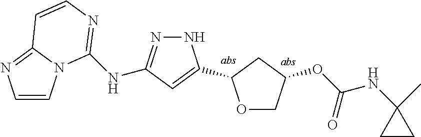 [SMILES: CC1(NC(=O)O[C@@H]2CO[C@H](c3cc(Nc4nccc5nccn45)n[nH]3)C2)CC1] | 27.71 |
| I-8 | 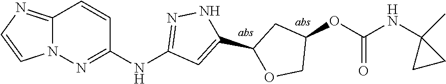 [SMILES: CC1(NC(=O)O[C@H]2CO[C@@H](c3cc(Nc4ccc5nccn5n4)n[nH]3)C2)CC1] | 27.56 |
| I-9 | 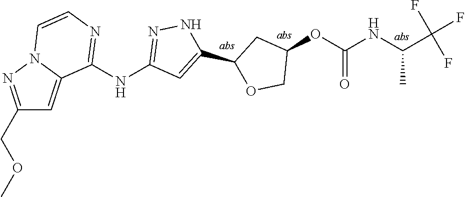 [SMILES: COCc1cc2c(Nc3cc([C@H]4C[C@@H](OC(=O)N[C@@H](C)C(F)(F)F)CO4)[nH]n3)nccn2n1] | 24.63 |
| I-10 | 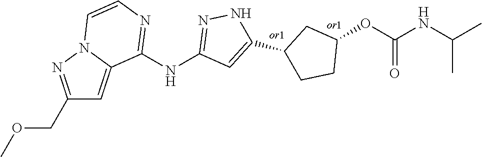 [SMILES: COCc1cc2c(Nc3cc([C@H]4CC[C@@H](OC(=O)NC(C)C)C4)[nH]n3)nccn2n1] | 24.36 |
| I-11 | 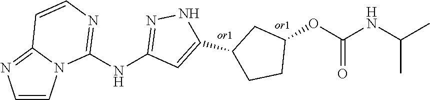 [SMILES: CC(C)NC(=O)O[C@@H]1CC[C@H](c2cc(Nc3nccc4nccn34)n[nH]2)C1] | 24.26 |
| I-12 | 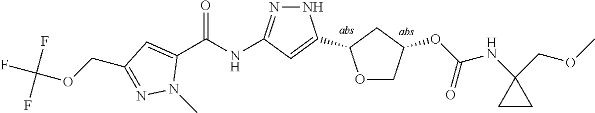 [SMILES: COCC1(NC(=O)O[C@@H]2CO[C@H](c3cc(NC(=O)c4cc(COC(F)(F)F)nn4C)n[nH]3)C2)CC1] | 23.98 |
| I-13 | 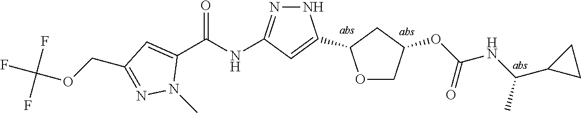 [SMILES: C[C@H](NC(=O)O[C@@H]1CO[C@H](c2cc(NC(=O)c3cc(COC(F)(F)F)nn3C)n[nH]2)C1)C1CC1] | 23.74 |
| I-14 | 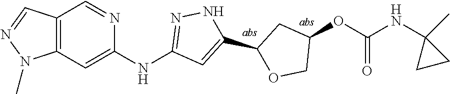 [SMILES: Cn1ncc2cnc(Nc3cc([C@H]4C[C@@H](OC(=O)NC5(C)CC5)CO4)[nH]n3)cc21] | 23.53 |
| I-15 | 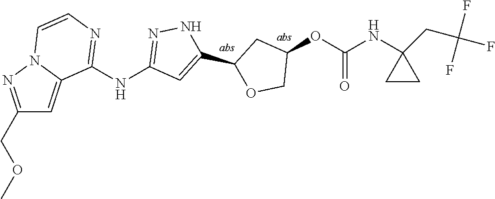 [SMILES: COCc1cc2c(Nc3cc([C@H]4C[C@@H](OC(=O)NC5(CC(F)(F)F)CC5)CO4)[nH]n3)nccn2n1] | 23.43 |
| I-16 | 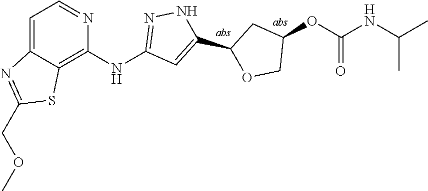 [SMILES: COCc1nc2ccnc(Nc3cc([C@H]4C[C@@H](OC(=O)NC(C)C)CO4)[nH]n3)c2s1] | 22.96 |
| I-17 | 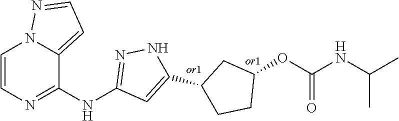 [SMILES: CC(C)NC(=O)O[C@@H]1CC[C@H](c2cc(Nc3nccn4nccc34)n[nH]2)C1] | 21.85 |
| I-18 | 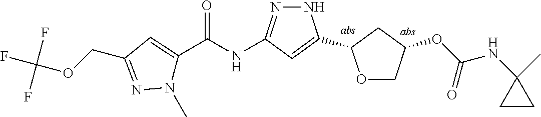 [SMILES: Cn1nc(COC(F)(F)F)cc1C(=O)Nc1cc([C@@H]2C[C@H](OC(=O)NC3(C)CC3)CO2)[nH]n1] | 21.63 |
| I-19 | 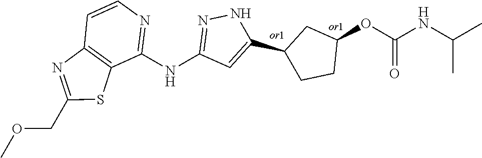 [SMILES: COCc1nc2ccnc(Nc3cc([C@@H]4CC[C@H](OC(=O)NC(C)C)C4)[nH]n3)c2s1] | 20.52 |
| I-20 | 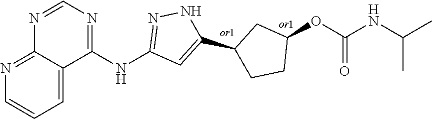 [SMILES: CC(C)NC(=O)O[C@H]1CC[C@@H](c2cc(Nc3ncnc4ncccc34)n[nH]2)C1] | 20.15 |
| I-21 | 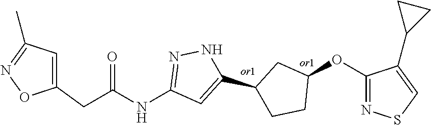 [SMILES: Cc1cc(CC(=O)Nc2cc([C@@H]3CC[C@H](Oc4nscc4C4CC4)C3)[nH]n2)on1] | 19.98 |
| I-22 | 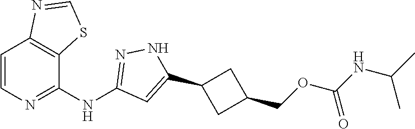 [SMILES: CC(C)NC(=O)OC[C@H]1C[C@@H](c2cc(Nc3nccc4ncsc34)n[nH]2)C1] | 19.69 |
| I-23 | 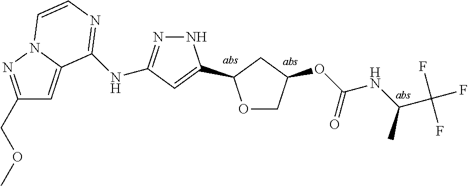 [SMILES: COCc1cc2c(Nc3cc([C@H]4C[C@@H](OC(=O)N[C@H](C)C(F)(F)F)CO4)[nH]n3)nccn2n1] | 19.67 |
| I-24 | 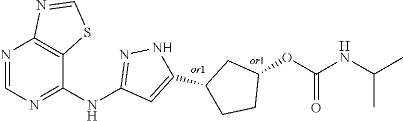 [SMILES: CC(C)NC(=O)O[C@@H]1CC[C@H](c2cc(Nc3ncnc4ncsc34)n[nH]2)C1] | 19.46 |
| I-25 | 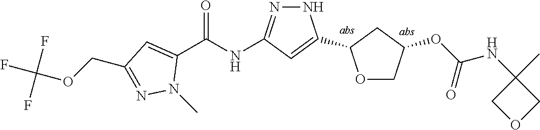 [SMILES: Cn1nc(COC(F)(F)F)cc1C(=O)Nc1cc([C@@H]2C[C@H](OC(=O)NC3(C)COC3)CO2)[nH]n1] | 18.78 |
| I-26 | 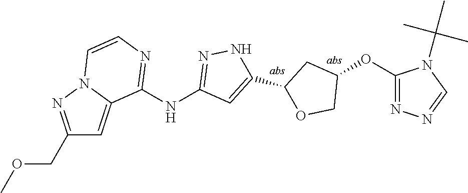 [SMILES: COCc1cc2c(Nc3cc([C@@H]4C[C@H](Oc5nncn5C(C)(C)C)CO4)[nH]n3)nccn2n1] | 18.65 |
| I-27 | 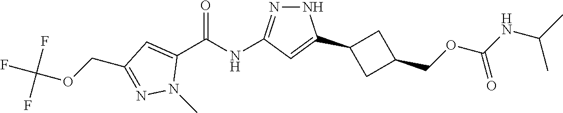 [SMILES: CC(C)NC(=O)OC[C@H]1C[C@@H](c2cc(NC(=O)c3cc(COC(F)(F)F)nn3C)n[nH]2)C1] | 18.56 |
| I-28 | 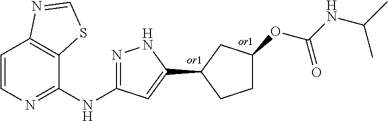 [SMILES: CC(C)NC(=O)O[C@H]1CC[C@@H](c2cc(Nc3nccc4ncsc34)n[nH]2)C1] | 18.45 |
| I-29 | 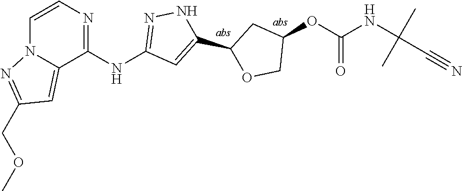 [SMILES: COCc1cc2c(Nc3cc([C@H]4C[C@@H](OC(=O)NC(C)(C)C#N)CO4)[nH]n3)nccn2n1] | 18.45 |
| I-30 | 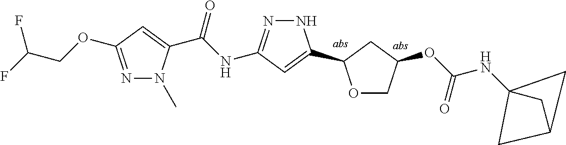 [SMILES: Cn1nc(OCC(F)F)cc1C(=O)Nc1cc([C@H]2C[C@@H](OC(=O)NC34CC(C3)C4)CO2)[nH]n1] | 18.27 |
| I-31 | 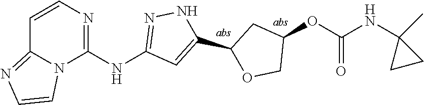 [SMILES: CC1(NC(=O)O[C@H]2CO[C@@H](c3cc(Nc4nccc5nccn45)n[nH]3)C2)CC1] | 17.88 |
| I-32 | 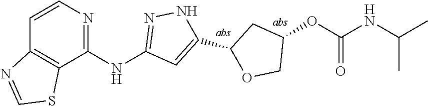 [SMILES: CC(C)NC(=O)O[C@@H]1CO[C@H](c2cc(Nc3nccc4ncsc34)n[nH]2)C1] | 17.60 |
| I-33 | 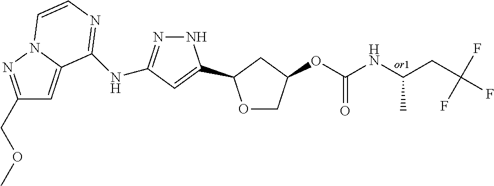 [SMILES: COCc1cc2c(Nc3cc([C@H]4C[C@@H](OC(=O)N[C@@H](C)CC(F)(F)F)CO4)[nH]n3)nccn2n1] | 17.57 |
| I-34 | 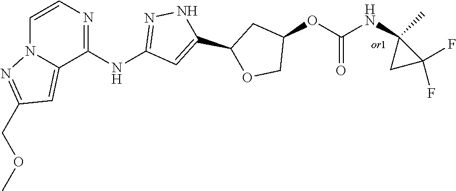 [SMILES: COCc1cc2c(Nc3cc([C@H]4C[C@@H](OC(=O)N[C@]5(C)CC5(F)F)CO4)[nH]n3)nccn2n1] | 17.33 |
| I-35 | 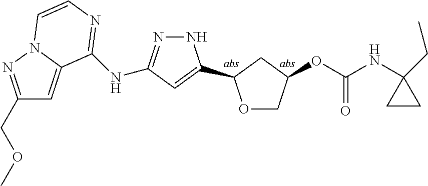 [SMILES: CCC1(NC(=O)O[C@H]2CO[C@@H](c3cc(Nc4nccn5nc(COC)cc45)n[nH]3)C2)CC1] | 17.20 |
| I-36 | 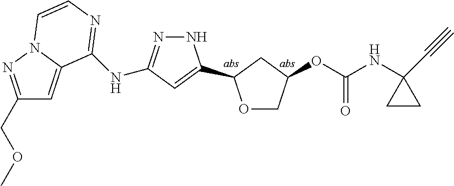 [SMILES: C#CC1(NC(=O)O[C@H]2CO[C@@H](c3cc(Nc4nccn5nc(COC)cc45)n[nH]3)C2)CC1] | 16.79 |
| I-37 | 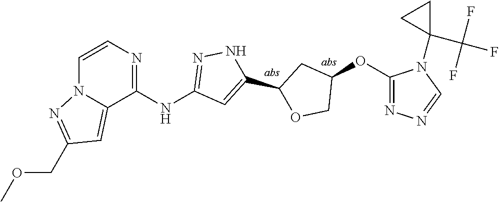 [SMILES: COCc1cc2c(Nc3cc([C@H]4C[C@@H](Oc5nncn5C5(C(F)(F)F)CC5)CO4)[nH]n3)nccn2n1] | 16.55 |
| I-38 | 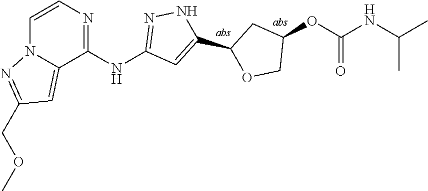 [SMILES: COCc1cc2c(Nc3cc([C@H]4C[C@@H](OC(=O)NC(C)C)CO4)[nH]n3)nccn2n1] | 16.55 |
| I-39 | 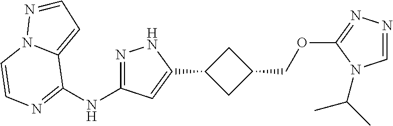 [SMILES: CC(C)n1cnnc1OC[C@H]1C[C@@H](c2cc(Nc3nccn4nccc34)n[nH]2)C1] | 16.17 |
| I-40 | 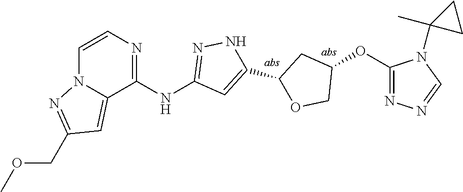 [SMILES: COCc1cc2c(Nc3cc([C@@H]4C[C@H](Oc5nncn5C5(C)CC5)CO4)[nH]n3)nccn2n1] | 16.06 |
| I-41 | 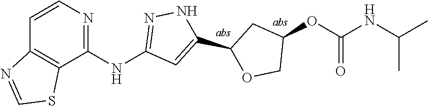 [SMILES: CC(C)NC(=O)O[C@H]1CO[C@@H](c2cc(Nc3nccc4ncsc34)n[nH]2)C1] | 15.98 |
| I-42 | 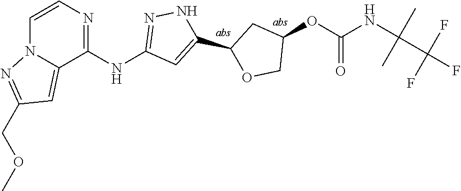 [SMILES: COCc1cc2c(Nc3cc([C@H]4C[C@@H](OC(=O)NC(C)(C)C(F)(F)F)CO4)[nH]n3)nccn2n1] | 15.77 |
| I-43 | 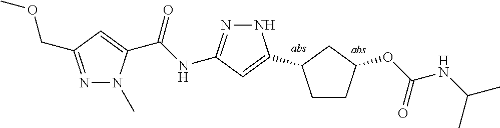 [SMILES: COCc1cc(C(=O)Nc2cc([C@H]3CC[C@@H](OC(=O)NC(C)C)C3)[nH]n2)n(C)n1] | 15.73 |

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A compound of formula I-A: 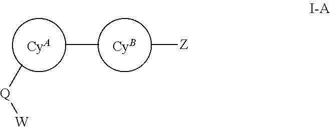
or a pharmaceutically acceptable salt thereof, wherein:
Q is L¹;
Cy^{A} is a 5-6 membered heteroarylene having 1-4 heteroatoms independently selected from nitrogen, oxygen, and sulfur; wherein Cy^{A} is substituted with m instances of R^{A} in addition to Q and Cy^{B};
Cy^{B} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenylene; or a 5-14 membered heteroarylene having 1-4 heteroatoms independently selected from nitrogen, oxygen, and sulfur; wherein Cy^{B} is substituted with n instances of R^{B} in addition to Cy^{A} and Z;
Z is hydrogen or L²-R^{Z};
R^{Z} is hydrogen, or an optionally substituted group selected from C₁₋₈ aliphatic, a saturated or partially unsaturated 3-14 membered carbocyclic ring, phenyl, a 3-10 membered saturated or partially unsaturated heterocyclic ring having 1-4 heteroatoms independently selected from nitrogen, oxygen, and sulfur, and a 5-14 membered heteroaryl ring having 1-4 heteroatoms independently selected from nitrogen, oxygen, and sulfur;
W is hydrogen or Cy^{C};
Cy^{C} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenyl; or a 5-14 membered heteroaryl having 1-4 heteroatoms independently selected from nitrogen, oxygen, and sulfur; wherein Cy^{C} is substituted with p instances of R^{C} in addition to Q;
each instance of R^{A}, RB, and R^{c} is independently R¹ or R², wherein R^{A} is substituted by q^{A} instances of R³, RB is substituted by q^{B} instances of R³, and R^{C} is substituted by q^{C} instances of R³; or
two instances of R^{A}, two instances of RB, two instances of R^{C}, an instance of R^{A} and an instance of R^{L}, or an instance of R^{C} and an instance of R^{L} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₅ saturated or unsaturated, straight or branched hydrocarbon chain wherein one or two methylene units of the chain are optionally and independently replaced by —CH(R^{L})—, —C(R^{L})₂-, C34 cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₋₆ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R^{1 is independently oxo}, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R;
each instance of R² is independently C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of m, n, p, q^{A}, q^{B}, q^{C}, q^{Z}, r, and t is independently 0, 1, 2, 3, or 4.

2. The compound of claim 1, wherein W is hydrogen.

3. The compound of claim 1, wherein W is Cy^{C}.

4. The compound of any one of claims 1-3, wherein Z is selected from —OR^{Z}, —NHR^{Z}—, —SR^{Z}, —NHC(O)NHR^{Z}, —OC(O)NHR^{Z}, and —NHC(O)OR^{Z}.

5. The compound of any one of claims 1-4, wherein R^{Z} is an optionally substituted group selected from C₁₋₆ aliphatic and a saturated or partially unsaturated 3-7 membered carbocyclic ring.

6. A compound of formula I: 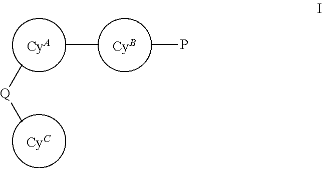
or a pharmaceutically acceptable salt thereof, wherein:
Q is L¹;
Cy^{A} is a 5-6 membered heteroarylene having 1-4 heteroatoms independently selected from nitrogen, oxygen, and sulfur; wherein Cy^{A} is substituted with m instances of R^{A} in addition to Q and Cy^{B};
Cy^{B} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenylene; or a 5-14 membered heteroarylene having 1-4 heteroatoms independently selected from nitrogen, oxygen, and sulfur; wherein Cy^{B} is substituted with n instances of R^{B} in addition to Cy^{A} and P;
P is hydrogen or —L²-R^{P};
RP is R;
Cy^{C} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenyl; or a 5-14 membered heteroaryl having 1-4 heteroatoms independently selected from nitrogen, oxygen, and sulfur; wherein Cy^{C} is substituted with p instances of R^{C} in addition to Q;
each instance of R^{A}, R^{B}, and R^{C} is independently R¹ or R², wherein R^{A} is substituted by q^{A} instances of R³, R^{B} is substituted by q^{B} instances of R³, and R^{C} is substituted by q^{C} instances of R³; or
two instances of R^{A}, two instances of R^{B}, two instances of R^{C}, an instance of R^{A} and an instance of R^{L}, or an instance of R^{C} and an instance of R^{L} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L¹ and L² is independently a covalent bond, or a C₁₋₅ saturated or unsaturated, straight or branched hydrocarbon chain wherein one or two methylene units of the chain are optionally and independently replaced by —CH(R^{L})—, —C(R^{L})₂—, C₃₄ cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₄ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R^{1 is independently oxo}, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R;
each instance of R² is independently C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of m, n, p, q^{A}, q^{B}, q^{C}, r, and t is independently 0, 1, 2, 3, or 4.

7. A compound of formula II, III, IV, V, VI, or VII: 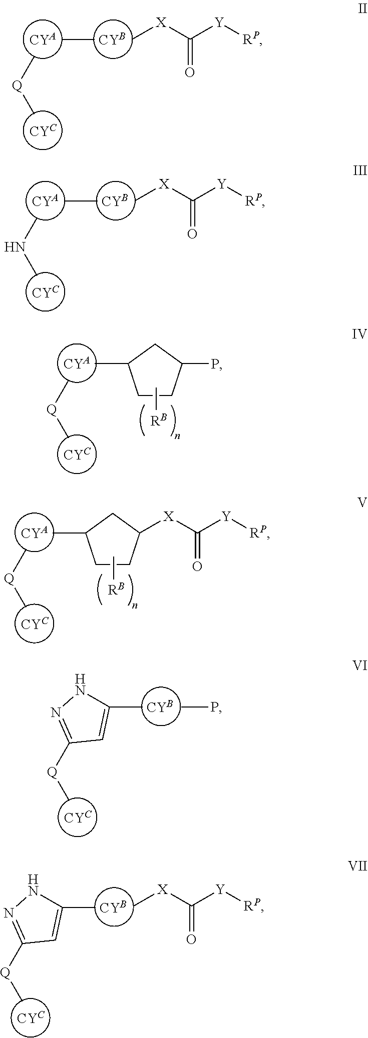
or a pharmaceutically acceptable salt thereof, wherein:
Q is L¹;
Cy^{A} is a 5-6 membered heteroarylene having 1-4 heteroatoms independently selected from nitrogen, oxygen, and sulfur; wherein Cy^{A} is substituted with m instances of R^{A} in addition to Q and Cy^{B};
Cy^{B} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenylene; or a 5-14 membered heteroarylene having 1-4 heteroatoms independently selected from nitrogen, oxygen, and sulfur; wherein Cy^{B} is substituted with n instances of R^{B} in addition to Cy^{A} and P or X;
X is selected from O, NR^{X}, and S;
Y is selected from O, NR^{Y}, and S;
each instance of R^{X} and R^{Y} is independently R;
P is hydrogen or —L²-R^{P};
RP is R;
Cy^{C} is a saturated or partially unsaturated 3-14 membered carbocyclic ring; a saturated or partially unsaturated 3-14 membered heterocyclic ring having 1-4 heteroatoms independently selected from nitrogen, oxygen, and sulfur; phenyl; or a 5-14 membered heteroaryl having 1-4 heteroatoms independently selected from nitrogen, oxygen, and sulfur; wherein Cy^{C} is substituted with p instances of R^{C} in addition to Q;
each instance of R^{A}, R^{B}, and R^{c} is independently R¹ or R², wherein R^{A} is substituted by q^{A} instances of R³, R^{B} is substituted by q^{B} instances of R³, and R^{C} is substituted by q^{C} instances of R³; or
two instances of R^{A}, two instances of R^{B}, two instances of R^{C}, an instance of R^{A} and an instance of R^{L}, or an instance of R^{C} and an instance of R^{L} are taken together with their intervening atoms to form a 4-8 membered saturated, partially unsaturated or aromatic ring having 0-4 heteroatoms independently selected from nitrogen, oxygen, and sulfur; wherein said ring is substituted with r instances of R³;
each of L and L² is independently a covalent bond, or a C₁₋₅ saturated or unsaturated, straight or branched hydrocarbon chain wherein one or two methylene units of the chain are optionally and independently replaced by —CH(R^{L})—, —C(R^{L})₂—, C₃₄ cycloalkylene, 3-6 membered heterocycloalkylene, 5-6 membered heteroarylene, —NH—, —N(R^{L})—, —NHC(O)—, —N(R^{L})C(O)—, —C(O)NH—, —C(O)N(R^{L})—, —NHS(O)₂—, —N(R^{L})S(O)₂—, —S(O)₂NH—, —S(O)₂N(R^{L})—, —O—, —C(O)—, —OC(O)—, —C(O)O—, —S—, —S(O)—, or —S(O)₂—; wherein each of said C₃₄ cycloalkylene, 3-6 membered heterocycloalkylene, and 5-6 membered heteroarylene is optionally substituted with one instance of R¹ or C₁₋₆ aliphatic;
each instance of R^{L} is independently R¹ or R², and is substituted by t instances of R³;
each instance of R^{1 is independently oxo}, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, or —N(R)S(O)₂R;
each instance of R² is independently C₁₋₇ aliphatic; phenyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R³ is independently oxo, halogen, —CN, —NO₂, —OR, —SR, —NR₂, —S(O)₂R, —S(O)₂NR₂, —S(O)R, —S(O)NR₂, —S(O)₂F, —OS(O)₂F, —C(O)R, —C(O)OR, —C(O)NR₂, —C(NR)NR₂, —C(O)N(R)OR, —OC(O)R, —OC(O)NR₂, —N(R)C(O)OR, —N(R)C(O)R, —N(R)C(NR)R, —N(R)C(O)NR₂, —N(R)C(NR)NR₂, —N(R)S(O)₂NR₂, —N(R)S(O)R, —N(R)S(O)₂R, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, a saturated or partially unsaturated 3-7 membered carbocyclic ring, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of m, n, p, q^{A}, q^{B}, q^{C}, r, and t is independently 0, 1, 2, 3, or 4.

8. The compound of any one of claims 1 or 2-7, wherein Cy^{C} is a 5-6 membered heteroaryl ring having 1-3 heteroatoms selected from nitrogen, oxygen, and sulfur.

9. The compound of any one of claims 1-8, wherein Q is selected from —NH—, 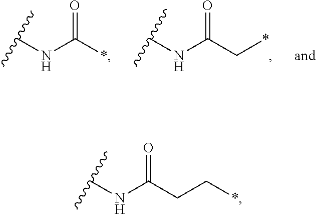
wherein 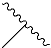
represents a covalent bond to Cy^{A} and [symbol] represents a covalent bond to W or Cy^{C}.

10. The compound of any one of claims 1-9, wherein Q is —NH—.

11. The compound of any one of claims 1-10, wherein Cy^{A} is a heteroarylene having 1-4 heteroatoms selected from nitrogen, oxygen, and sulfur.

12. The compound of any one of claims 1-11, wherein Cy^{A} is selected from: 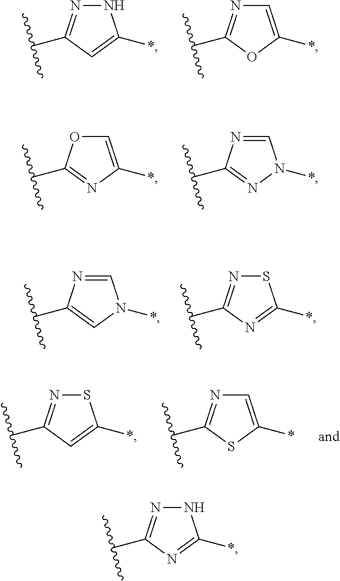
wherein 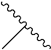
represents a covalent bond to Q and [symbol] represents a covalent bond to Cy^{B}.

13. The compound of any one of claims 1-12, wherein Cy^{B} is a bivalent 3-7 membered saturated or partially unsaturated carbocyclic ring.

14. The compound of any one of claims 1-12, wherein Cy^{B} is selected from: 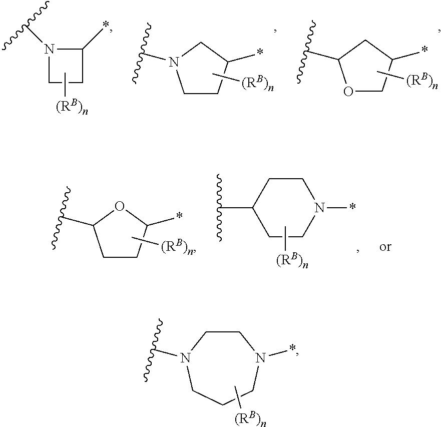
wherein 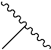
represents a covalent bond to Cy^{A} and [symbol] represents a covalent bond to P, X, or Z.

15. The compound of any one of claims 1-14, wherein R^{B} is selected from —CH₃, —CH₂CH₃, —CH(CH₃)₂,

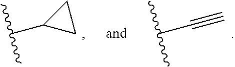

16. The compound of any one of claims 1-15, wherein n is 0, 1, 2, 3, or 4

17. The compound of any one of claims 2-16, wherein P is selected from —OR^{P}, —NHR^{P}—, —SR^{P}, —NHC(O)NHR^{P}, —OC(O)NHR^{P}, and —NHC(O)OR^{P}.

18. The compound of any one of claims 2-17, wherein R^{P} is an optionally substituted group selected from C₁₋₆ aliphatic and a saturated or partially unsaturated 3-7 membered carbocyclic ring.

19. The compound of any one of claims 1-18, wherein the compound is selected from those depicted in Table 1, or a pharmaceutically acceptable salt thereof.

20. A pharmaceutical composition, comprising a compound of any one of claims 1-19, and a pharmaceutically acceptable carrier.

21. A method of inhibiting CDK2 signaling activity in a subject, comprising administering a therapeutically effective amount of a compound of any one of claims 1-19, or the pharmaceutical composition of claim 20, to a subject in need thereof.

22. A method of treating an CDK2-mediated disorder in a subject, comprising administering a therapeutically effective amount of a compound of any one of claims 1-19, or the pharmaceutical composition of claim 20, to a subject in need thereof.

23. A method of treating a disorder in a subject, wherein the disorder is ovarian cancer, endometrial cancer, gastric cancer, breast cancer, lung cancer, bladder cancer, cervical cancer, stomach cancer, sarcoma cancer, liver cancer, esophageal cancer, laryngeal cancer, multiple myeloma, colorectal cancer, rectal cancer, skin cancer, or pancreatic cancer, the method comprising administering a therapeutically effective amount of a compound of any one of claims 1-19, or the pharmaceutical composition of claim 20, to a subject in need thereof.

24. The method of claim 23, wherein the bladder cancer is urothelial carcinoma.

25. The method of claim 23, wherein the liver cancer is hepatocellular carcinoma.

26. The method of claim 23, wherein the lung cancer is lung squamous cell carcinoma or non-small cell lung cancer.

27. The method of claim 23, wherein the laryngeal cancer is laryngeal squamous cell carcinoma.

28. The method of claim 23, wherein the skin cancer is melanoma.


SEQUENCE LISTING (1 sequence)

SEQ ID NO: 1 - protein, 6 aa, Artificial Sequence
  Description of Artificial Sequence Synthetic peptide
QSPKKG